SEQUENCE LISTING (1 sequence)

SEQ ID NO: 1 - protein, 18 aa, Artificial Sequence
  synthetic
LNIDLDLVLSTASINFQK

(19) United States
(12) Patent Application Publication
(10) Pub. No.: US 2024/0109900 A1
(43) Pub. Date: Apr. 4, 2024

(54) AZABICYCLIC SHP2 INHIBITORS

(71) Applicants: IRBM S.P.A., Pomezia (RM) (IT); C.N.C.C.S. S.C.A.R.L. COLLEZIONE NAZIONALE DEI COMPOSTI CHIMICI E CENTRO SCREENING, Pomezia (RM) (IT)

(72) Inventors: Alina CIAMMAICHELLA, Pomezia (RM) (IT); Romano DI FABIO, Pomezia (RM) (IT); Federica FERRIGNO, Pomezia (RM) (IT); Paola FEZZARDI, Pomezia (RM) (IT); Christian MONTALBETTI, Pomezia (RM) (IT); Alessia PETROCCHI, Pomezia (RM) (IT); Nicola RELITTI, Pomezia (RM) (IT); Ilaria ROSSETTI, Pomezia (RM) (IT); Alessio SFERRAZZA, Pomezia (RM) (IT); Esther TORRENTE, Pomezia (RM) (IT); Luca FERRANTE, Milano (MI) (IT); Alessandro GRILLO, Milano (MI) (IT)

(21) Appl. No.: 18/264,299

(22) PCT Filed: Feb. 8, 2022
(86) PCT No.: PCT/EP2022/053003

§ 371 Date: Aug. 4, 2023

(30) Foreign Application Priority Data
Feb. 8, 2021 (EP) 21155769.9

(51) Int. Cl. C07D 487/04; A61K 45/06; C07D 401/04; C07D 401/14; C07D 409/14; C07D 417/14; C07D 471/04; C07D 519/00

(52) U.S. Cl. CPC C07D 487/04; A61K 45/06; C07D 401/04; C07D 401/14; C07D 409/14; C07D 417/14; C07D 471/04; C07D 519/00

(57) ABSTRACT

The present invention relates to new compounds capable of inhibiting the activity of SHP2 phosphatase, having the general Formula (I).

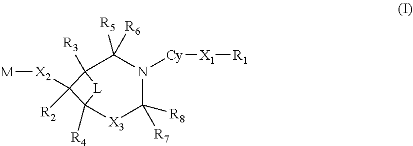

### FIELD OF THE INVENTION

The present invention relates to new compounds capable of inhibiting the activity of SHP2 phosphatase. Compounds of the invention can be used for the treatment of disorders associated with SHP2 deregulation. The present invention also relates to pharmaceutical compositions containing said compounds and to their method of manufacture.

### BACKGROUND OF THE INVENTION

Src homology phosphotyrosine phosphatase 2 (SHP2) encoded by PTPN11 is a non-receptor protein tyrosine phosphatase (PTP) composed of a C-terminal domain, a PTP domain, and two N-terminal Src homology (N—SH2) domains, that contributes to multiple cellular functions including proliferation, differentiation, cell cycle maintenance and migration. SHP2 is a positive regulator of signalling downstream of several receptor tyrosine kynases through the Ras-mitogen-activated protein kinase, the JAK-STAT or the phosphoinositol-3-kinase-AKT pathways. The protein exists in an inactive, self-inhibited conformation, stabilized by a binding network involving residues from both the N—SH2 domains and the catalytic PTP domain. Recruitment of SHP2 to an activated receptor releases the self-inhibitory conformation and leads to catalytic activation of its phosphatase domain. In addition to its function as a phosphatase, SHP2 also serves as a docking protein to recruit other signalling intermediates through its two amino terminus N—SH2 domains. Since SHP2 is a positive regulator of cellular signalling leading to proliferation, differentiation, and survival, its constitutive activation is associated with oncogenesis. SHP2 emerged as an attractive target for therapeutic targeting in the treatment of various diseases, such as Noonan Syndrome, Leopard Syndrome, juvenile myelocytic leukemias, neuroblastoma, melanoma, acute myeloid leukemia and cancers of the breast, lung and colon.

Both academic institutions and pharmaceutical companies have disclosed drug discovery programs exploiting SHP2 inhibitors based on different heterocyclic scaffolds.

WO2015/107493, WO2015/107494 and WO2015/107495 from Novartis disclose compounds of general formula (A) as indicated below:

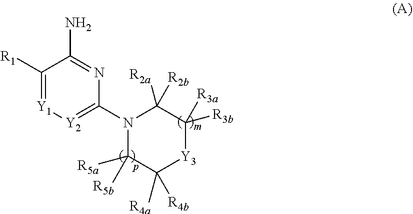

Still from Novartis the compounds of general structures (B), (C), (D) and (E) indicated below are disclosed in, respectively, WO2016/203404, WO2016/203405 and WO2017/216706:

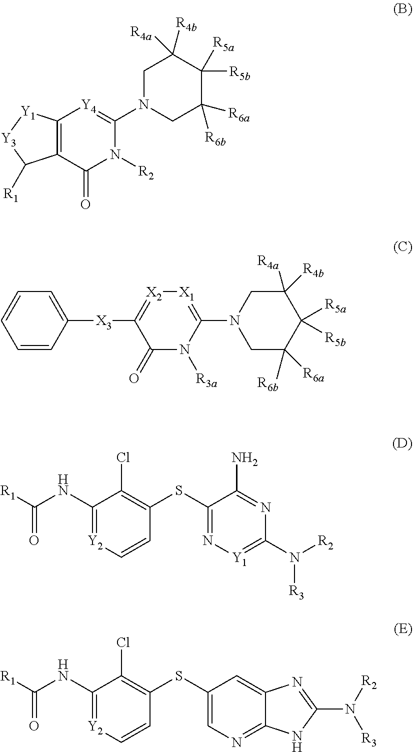

The general structure E disclosed in WO2017/216706 and the compounds therein identified are 2-amino-3H-imidazo[4,5-b]pyridine 5- or 6-thiol derivatives.

Jacobio Pharmaceuticals disclosed in WO2017/211303 and in WO2018/172984 pyrazine derivatives of structures (F) and (G) as indicated below:

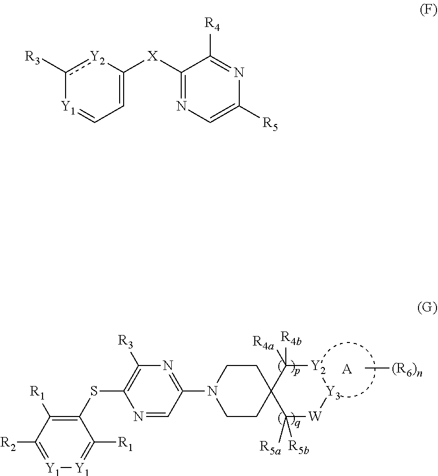

Further pyridine, pyrazine and triazine compounds as allosteric SHP2 inhibitors have been recently disclosed by Revolution Medicines in WO2018/013597, WO2018/136264 and WO2019/075265.

WO2018/136265 and WO2019/118909 both relate to bicyclic heteroaromatic scaffolds comprising imidazopyrazines, triazolopyrazines, pyrazolopyridine, imidazopyrimidines of general structure (H):

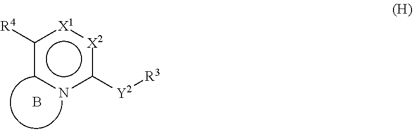

Pyrazolopyrazines and ring-fused pyrimidin-4-ones have been disclosed by the Board of Regents, University of Texas System, in WO2017/210134 and in WO2017/156397, respectively.

Pyrazolopyrazines were further disclosed by Relay Therapeutics in WO2018/081091, WO2018/218133, WO2018/057884 and WO2019/067843.

Imidazopyrimidine indicated below are disclosed by Gilead in WO2020/072656:

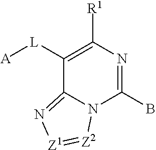

SHP2 therefore represents a highly attractive target for the development of novel therapies for the treatment of various diseases where it is involved. Therefore, there is the need to develop novel therapeutic agents that act as SHP2 inhibitors. The compounds of the present invention fulfil such need since they are small molecules capable of inhibiting the activity of SHP2.

### DESCRIPTION OF THE INVENTION

The present invention relates to heterocyclic compounds useful as SHP2 inhibitors and for the treatment of conditions mediated by SHP2.

The inventors have found that compounds having a specific general formula surprisingly act as potent SHP2 inhibitors, as evidenced by both enzymatic and cellular IC₅₀ values for SHP2 in the low nanomolar or micromolar range. For the compounds of the invention, the azabicyclic structure on the left hand side of the molecule is critical for potency and physicochemical properties.

It is therefore an object of the present invention a compound of general formula (I):

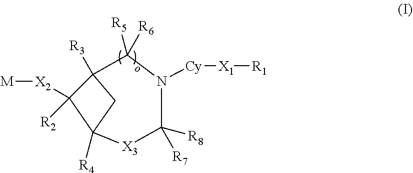

wherein
X₁ is S, O, CR₁ₐR_{1b}, NH, N—CH₃ or a single bond;
X₂ is (CR₂ₐR_{2b})ₘ;
X₃ is (CR₃ₐR_{3b})ₙ;
M is NH₂, NH—C₁₋₆alkyl, N(C₁₋₆alkyl)₂, NHC(═O)Rₓ, or NHC(═O)ORₓ, wherein Rₓ, is C₁₋₆alkyl, aryl, heteroaryl or C₁₋₆alkyl-aryl;
L is a single bond or C₁₋₃alkyl;
Cy-X₁—R₁ has general formula (A):

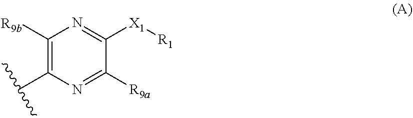

wherein
   R₉ₐ is selected from H, NH₂, C₁₋₆alkyl, halogen, cyano, C₃₋₅cycloalkyl, haloC₁₋₆alkyl, hydroxy-C₁₋₆alkyl or aminoC₁₋₆alkyl;
   R_{9b} is selected from H, C₁₋₆alkyl, halogen, haloC₁₋₆alkyl, hydroxy-C₁₋₆alkyl or aminoC₁₋₆alkyl;
or Cy-X₁—R₁ has general formula (B):

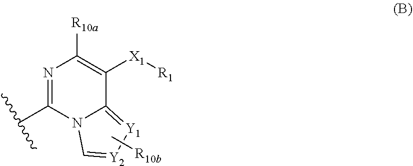

wherein
   Y₁ and Y₂ are C or N;
   R₁₀ₐ is H or C₁₋₆alkyl;
   R_{10b} is at each occurrence independently H, C₁₋₆alkyl, NH₂, CN, OH, haloC₁₋₃alkyl, C₁₋₃alkoxy, haloC₁₋₃alkoxy, or halogen;
or Cy-X₁—R₁ has general formula (C):

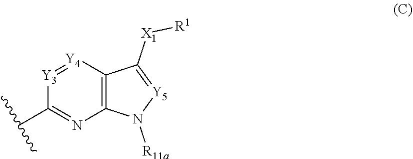

wherein
   Y₃ is C—R_{11b} or N;
   Y₄ is C—R_{11b} or N; or Y₃ is N—CH₃ and Y₄ is C(═O);
   Y₅ is C-Rue. or N;
   Y [symbol] Y₄ is a double bond when Y₃ is C—R_{11b} or N and when Y₄ is C—R_{11b} or N; Y₃ [symbol] Y₄ is a single bond when Y₃ is N—CH₃ and Y₄ is C(═O);
   R₁₁ₐ is H or methyl;
   R_{11b} is H, C₁₋₆alkyl, hydroxyC₁₋₆alkyl;
   R_{11c} is H, C₁₋₆alkyl, halogen;
or Cy-X₁—R₁ has general formula (D):

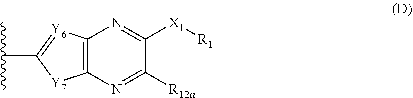

wherein
   Y₆ is N, CH or C—CH₃;
   Y₇ is N—R_{12b}, O or S;
   R₁₂ₐ is H, C₁₋₆alkyl, halogen or cyano;
   R_{12b} is H or C₁₋₆alkyl;
or Cy-X₁—R₁ has general formula (E):

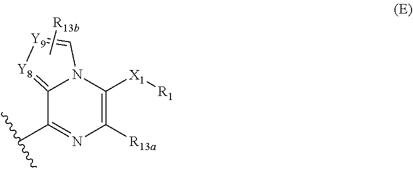

wherein
   Y₈ and Y₉ are C or N;
   R₁₃ₐ is H, C₁₋₆alkyl, halogen, NH₂ or cyano;
   R_{13b} is at each occurrence independently H, CH₃ or hydroxyC₁₋₆alkyl;
or Cy-X₁—R₁ has general formula (F):

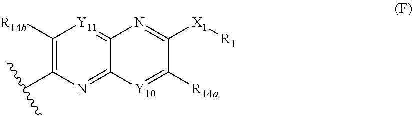

wherein
   Y₁₀ and Y₁₁ are C or N;
   R₁₄ₐ is H, C₁₋₆alkyl, halogen;
   R_{14b} is H, C₁₋₆alkyl or hydroxyC₁₋₆alkyl;
or Cy-X₁—R₁ has general formula (G):

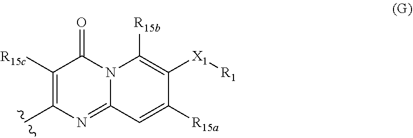

wherein
   R₁₅ₐ and R_{15b} are each independently H, C₁₋₆alkyl, halogen;
   R_{15c} is H, C₁₋₆alkyl or hydroxyC₁₋₆alkyl;
or Cy-X₁—R₁ has general formula (H):

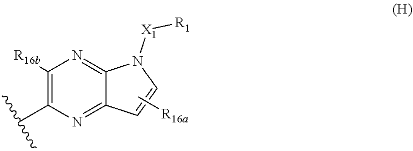

wherein
   R₁₆ₐ is H, C₁₋₆alkyl or halogen;
   R_{16b} is H, C₁₋₆alkyl or hydroxyC₁₋₆alkyl;
or Cy-X₁—R₁ has general formula (I):

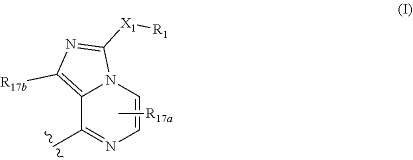

wherein
   R₁₇ₐ is H, C₁₋₆alkyl or halogen;
   R_{17b} is H, C₁₋₆alkyl or hydroxyC₁₋₆alkyl;
or Cy has general formula (J):

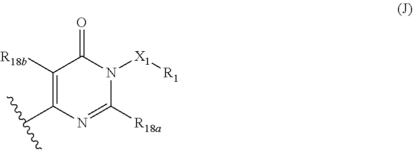

wherein
   R₁₈ₐ is H, C₁₋₆alkyl, haloC₁₋₆alkyl or halogen;
   R_{18b} is H, halogen, C₁₋₆alkyl or hydroxyC₁₋₆alkyl;
R₁ is an aryl, heteroaryl, partially unsaturated aryl, partially unsaturated heteroaryl or an heterocyclic ring, each of said aryl, heteroaryl, partially unsaturated aryl, partially unsaturated heteroaryl or heterocyclic ring being optionally substituted with one or more substituents independently selected from C(O)CH₃, C(O)OCH₃, heteroaryl, aryl, OH, halogen, NH₂, NH—C₁₋₆alkyl, N(C₁₋₆alkyl)₂, NH—C₃₋₆cycloalkyl, C(O)NH₂, C(O)NHC₁₋₃alkyl, C(O)N(C₁₋₃alkyl)₂, C₁₋₆alkylC(O)NH₂, C(O)NHSO₂CH₃, C(O)NHCN, C(O)N(CH₃)CN, SO₂NH₂, OC(O)CH₃, C₁₋₆alkyl, C₃₋₅cycloalkyl, C₁₋₆alkyl-NH₂, C₁₋₆alkyl-NH(CH₃), C₁₋₆alkyl-NH(CH₃)₂, C₁₋₆alkyl-NHCOCH₃, C₁₋₃alkylaryl, C₁₋₃alkylheteroaryl, haloC₁₋₆alkyl, hydroxyC₁₋₆alkyl, CN, haloC₁₋₆alkoxy, C₁₋₆alkoxy, C₅₋₇heterocycloalkoxy, oxetane, OCF₃, CH₂OCH₃, OSO₂F, SO₂F, C₁₋₃alkyl-OC₁₋₃alkyl, heterocycloalkyl, heteroaryl-OH, and a cyclic amine selected from the group consisting of: pyrrolidine, piperidine, morpholine, thiomorpholine, piperazine, N-methylpiperazine and pyrrolidin-3-yloxy, pyrrolidine-2-one wherein said cyclic amine is optionally substituted with C₁₋₃alkyl, C₁₋₃alkyl-OH; or R₁ has general formula (K)

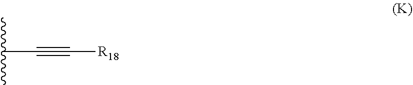

wherein R₁₈ is C₁₋₆alkyl, aryl, heteroaryl and said aryl or heteroaryl are optionally substituted with one or more substituents independently selected from halogen, hydroxy, C₁₋₃alkyl, C₁₋₃alkoxy;
R₂ is aryl, heteroaryl, C₃₋₉cycloalkyl, partially unsaturated heteroaryl, C₁₋₃alkylaryl, C₁₋₃alkylheteroaryl, each of said aryl, heteroaryl, C₃₋₉cycloalkyl, partially unsaturated heteroaryl, C₁₋₃alkylaryl, C₁₋₃alkylheteroaryl being optionally substituted with one or more substituents independently selected from the group consisting of: halogen, CN, C₁₋₃alkyl, C₃₋₈cycloalkyl, haloC₁₋₃alkyl, C₁₋₃alkoxy, haloC₁₋₃alkoxy, C₁₋₃alkyl-N(R_{y})₂, C(O)OC₁₋₃alkyl, C(O)C₁₋₃alkyl, OH, hydroxy-C₁₋₃alkyl, C₁₋₃alkyl-OC₁₋₃alkyl, CON(R_{y})₂, N(R_{y})₂ wherein each R_{y} is independently selected from H, methyl, ethyl and cyclopropyl; or R₂ is C(O)NH₂, C(O)NHC₁₋₃alkyl or C(O)N(C₁₋₃alkyl)₂;
R₁ₐ and R_{1b} are independently selected from H, C₁₋₆alkyl or R₁ₐ and R_{1b} are joined together to form together with the carbon atom to which they are linked a spiro-C₃₋₅cycloalkyl ring;
R₂ₐ and R_{2b} are independently selected from H, halogen, C₁₋₆alkyl, C₃₋₅cycloalkyl or R₃ₐ and R_{3b} are joined together to form together with the carbon atom to which they are linked a spiro-C₃₋₅cycloalkyl ring;
R₃, R₃ₐ, R_{3b}, R₄, R₅, R₆, R₇ and R₈ are at each occurrence independently selected from H, methyl or halogen;
m is 0, 1, 2 or 3;
n is 0, 1 or 2;
o is 1 or 2; preferably o is 1;
or a pharmaceutically acceptable salt, tautomer, solvate, or stereoisomer thereof.

In a further embodiment, in the compound of general formula (I) as above defined R₂ is H, aryl, heteroaryl, C₃₋₉cycloalkyl, partially unsaturated heteroaryl, C₁₋₃alkylaryl, C₁₋₃alkylheteroaryl, each of said aryl, heteroaryl, C₃₋₉cycloalkyl, partially unsaturated heteroaryl, C₁₋₃alkylaryl, C₁₋₃alkylheteroaryl being optionally substituted with one or more substituents independently selected from the group consisting of halogen, CN, C₁₋₃alkyl, C₃₋₈cycloalkyl, haloC₁₋₃alkyl, C₁₋₃alkoxy, haloC₁₋₃alkoxy, C₁₋₃alkyl-N(R_{y})₂, C(O)OC₁₋₃alkyl, C(O)C₁₋₃alkyl, OH, hydroxy-C₁₋₃alkyl, C₁₋₃alkyl-OC₁₋₃alkyl, CON(R_{y})₂, N(R_{y})₂ wherein each R_{y} is independently selected from H, methyl, ethyl and cyclopropyl; or R₂ is C(O)NH₂, C(O)NHC₁₋₃alkyl or C(O)N(C₁₋₃alkyl)₂; and if n is 0 then Cy doesn't have the general structure (B).

In a preferred embodiment compounds of the invention have general formula (II), (III), (IV), (V), (VI), (VII), (VIII), (IX), (X), (XI), (XII), (XIII), (XIV), (XV), (XVIa) or (XVIb):

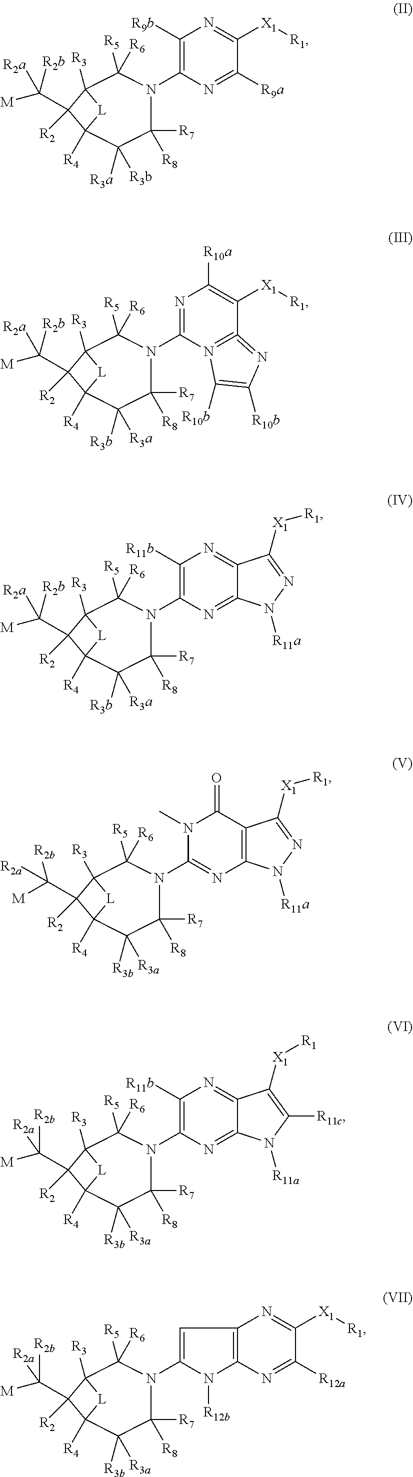

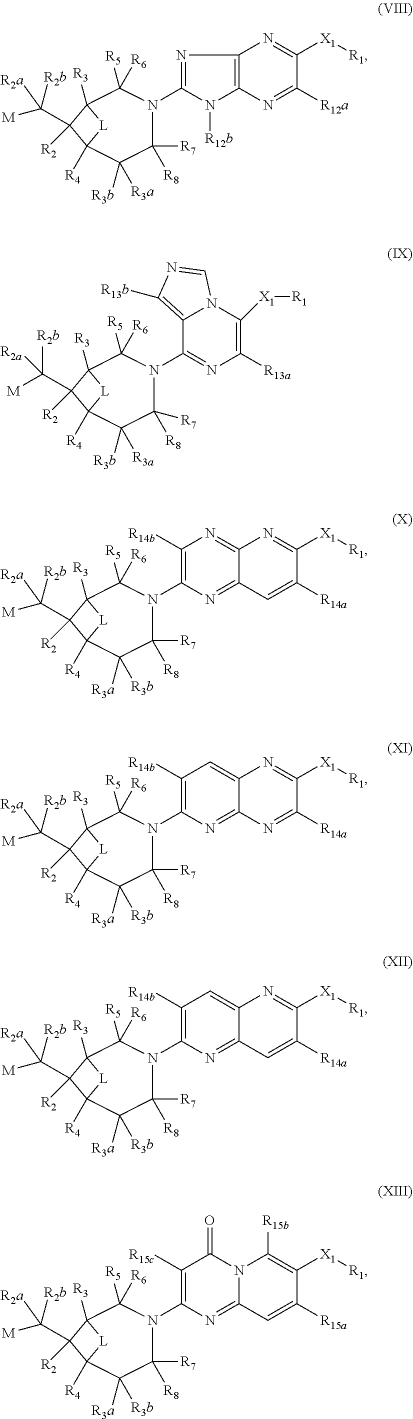

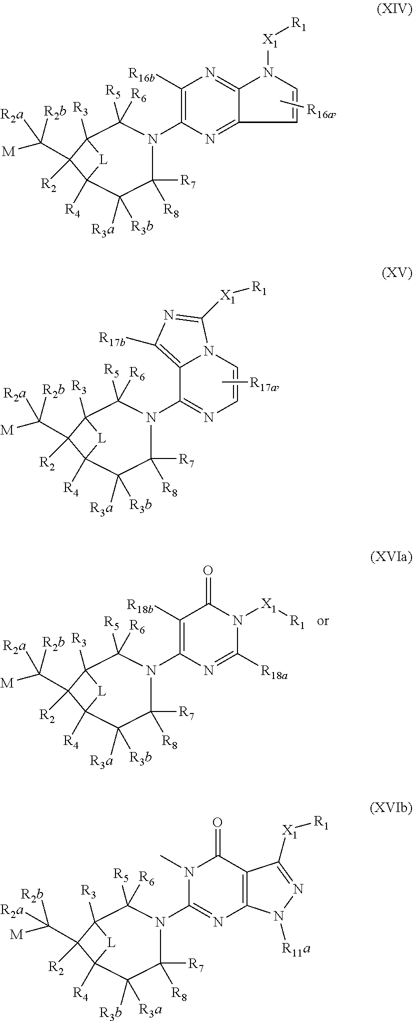

wherein: X₁, R₁, R₂, R₂ₐ, R_{2b}, R₃, R₃ₐ, R_{3b}, R₄, R₅, R₆, R₇, R₈, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R_{15c}, R₁₆ₐ, R_{16b}, R₁₇ₐ, R_{17b}, R₁₈ₐ, R_{18b}, M and L are as defined above.

In a further preferred embodiment compounds of the invention have general (XVII), (XVIII), (XIX), (XX), (XXI), (XXII), (XXIII), (XXIV), (XXV), (XXVI), (XXVII), (XXVIII), (XIX), (XXX), (XXXI) or (XXXII):

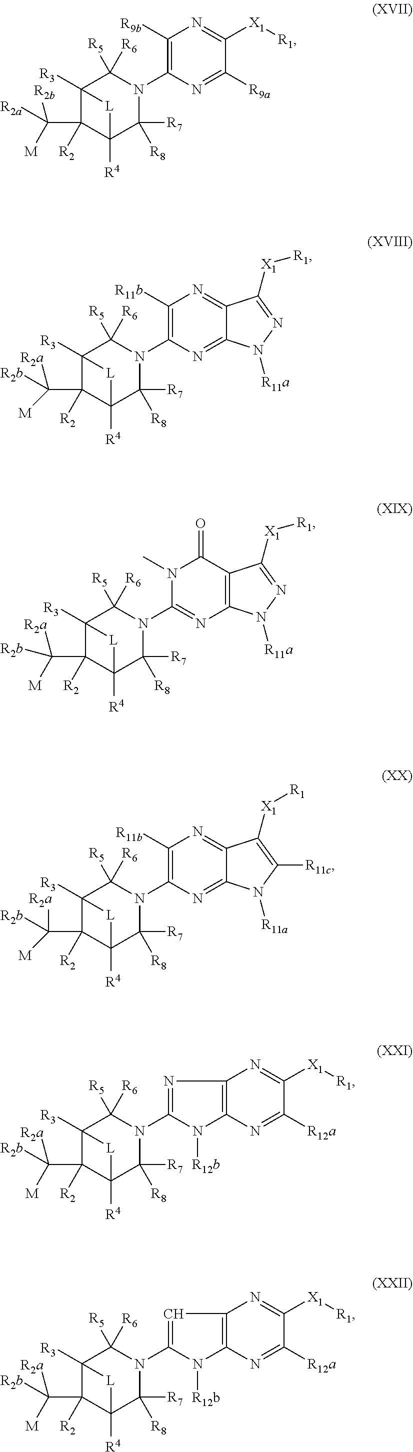

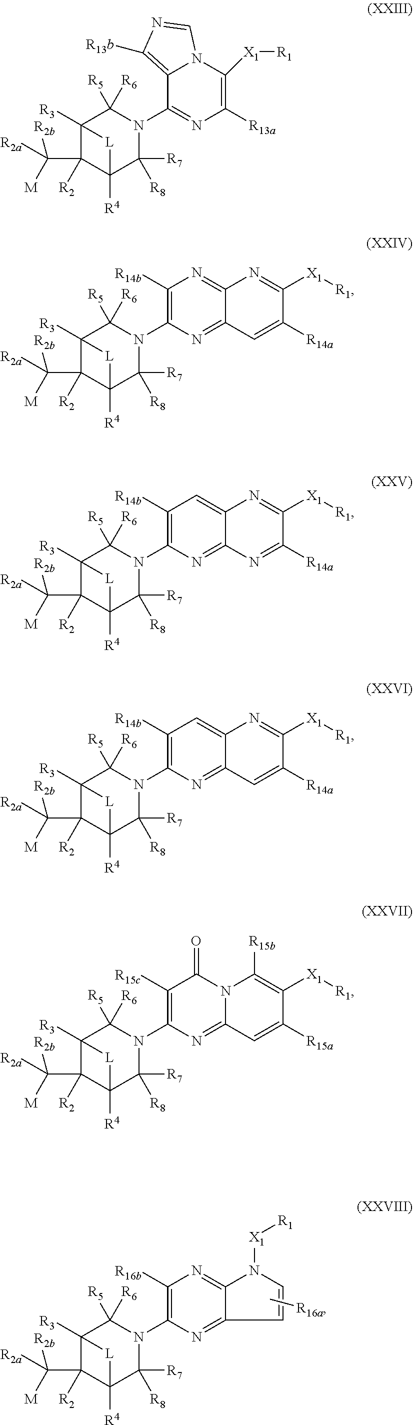

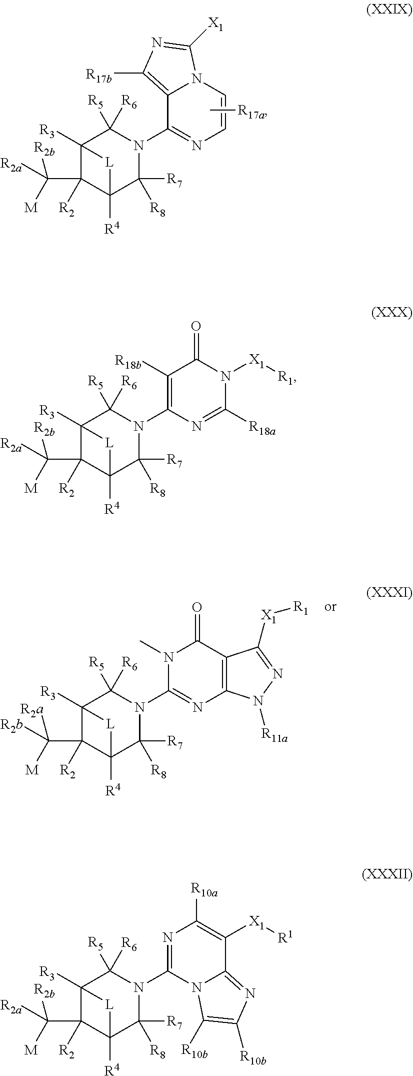

wherein: X₁, R₁, R₂, R₂ₐ, R_{2b}, R₃, R₄, R₅, R₆, R₇, R₈, R₉ₐ, R_{9b}, R₁₀ₐ, R_{10b}, R₁₁ₐ, R_{11b}, R_{11c}, R₁₂ₐ, R_{12b}, R₁₃ₐ, R_{13b}, R₁₄ₐ, R_{14b}, R₁₅ₐ, R_{15b}, R_{15c}, R₁₆ₐ, R_{16b}, R₁₇ₐ, R_{17c}, R₁₈ₐ, R_{18b}, M and L are as defined above.

In a further preferred embodiment in the compounds of the invention:
L is a single bond; and/or
X₁ is a single bond or S.

Still preferably M is NH₂; even more preferably R₂ₐ, R_{2b}, R₃, R₃ₐ, R_{3b}, R₄, R₅, R₆, R₇, R₈ are H.

In a further embodiment the invention relates to a compound of general formula (I-A):

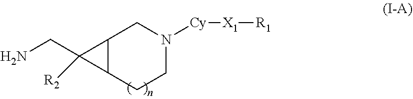

wherein n is 0 or 1; Cy-X₁—R₁ and R₂ are as defined above.

R₁ is preferably selected from: phenyl, napthyl, benzothiophene, quinoline, quinoxaline, isoquinoline, pyrazolo[3,4-b]pyrazine, pyridine, pyrimidine, pyrazine, thienopyridine, indole, indoline, 2,3-dihydroisoindol-1-one, indol-2-one, benzothiazole, 6a,7,8,9-tetrahydro-6H-pyrido[3,2-b]pyrrolo[1,2-d][1,4]oxazine, napthyridine, 1,2,3,4-tetrahydro-1,5-naphthyridine, benzo[d][1,3]dioxole, 2,3-dihydrobenzo[b][1,4]dioxine, benzoxazole, thieno[3,2-b]pyridine, indazole, furo[2,3-b]pyridine, 2,1,3-benzoxadiazole, benzo[d]oxazol-2(3H)-one, pyridine-2(1H)one, benzo[d][1,2,3]triazole, 1,4-benzoxazin-3(4H)-one, isoquinolin-(2H)one, quinolin-(2H)one, [1,2,4]triazolo[4,3-a]pyridine, imidazo[1,2-a]pyridine, 1,3-dihydro-2H-benzimidazol-2-one, benzoisothiazole-3(2H)-one-1,1-dioxide, benzo[d]thiazol-2(3H)-one phthalazine-1(2H)-one, pyrido[1,2-a]pyrimidin-4-one, cinnoline, [1,2,4]triazolo[1,5-a]pyridine, benzimidazole, indolin-2,3-dione, each being optionally substituted with one or more substituents independently selected form halogen, C₁₋₃alkyl, CN, CF₃, CHF₂, NH₂, OCH₃, OCF₃, CH₂OCH₃, OSO₂F, SO₂F, oxetane, C(CH₃)₂OH, CH(CH₃)OH, CH(CH₃)OCH₃, heteroaryl, heteroaryl-C₁₋₃alkyl-substituted, heteroaryloxy, C(O)N(CH₃)₂, C(O)NH₂, C(O)NHCH₃, C(O)NHcyclopropyl, C(O)NHCN, C(O)N(CH₃)CN CONHSO₂CH₃, C(O)OCH₃, COOH, cyclobutane, OH, CH₃C(O), CH₂C(O)NH₂, CH₂C(O)NH(CH₃), CH₂C(O)N(CH₃)₂, pyrrolidine-2-one, pyrrolidine-3-yl-methanol, CH₂CH₂—OH, CH₂CH₂—OCH₃, SO₂NH₂, OC(O)CH₃, CH₂CF₃, CH₂CN.

R₂ is preferably selected from: phenyl, pyridine, pyrimidine, pyrazine, oxazole, isoxazole, pyrrole, pyrazole triazole, oxadiazole, thiophene, thiazole, thiadiazole, imidazole, each optionally substituted with one or more substituents independently selected form halogen, C₁₋₃alkyl, CN, CF₃, NH₂, OCH₃, OH, CH₂OH, CH₂OCH₃, CO₂CH₃, CONHC₁₋₆alkyl; or R₂ is CON(CH₃)₂ or C(O)NHCH₃.

Still preferably R₁ is:

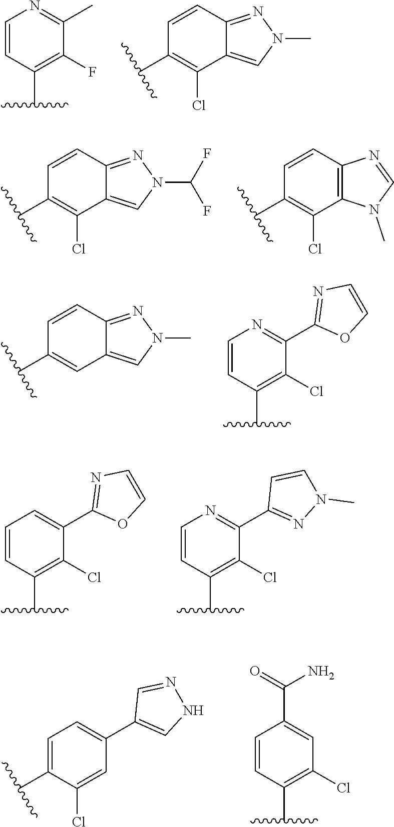

Still preferably R₂ is:

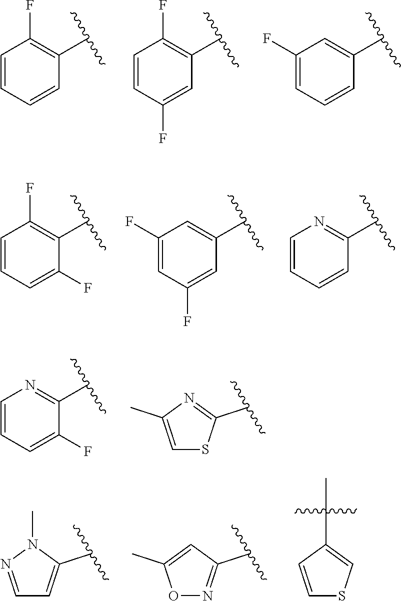

In certain embodiments, compounds have structural formula I-AA or I-AA′:

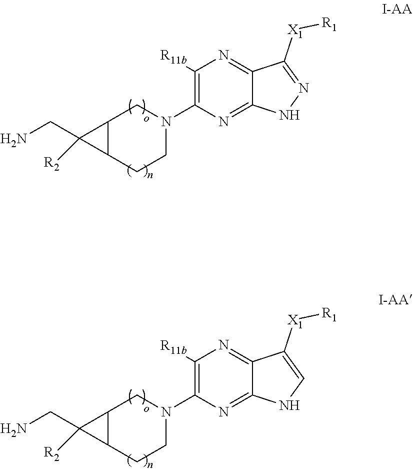

wherein n is 0 or 1;
R_{11b} is H, CH₂OH, CH₃, CH₂CH₃;
X₁, R₁ and R₂ are as defined above; preferably X₁ is a single bond or S.

In certain embodiments, compounds have structural formula I-AB or I-AB′:

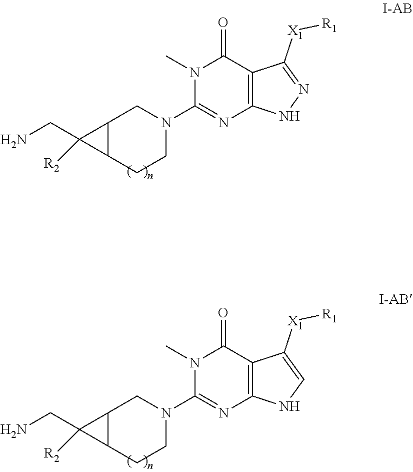

wherein n is 0 or 1; X₁, R₁ and R₂ are as defined above; preferably X₁ is a single bond or S.

In certain embodiments, compounds have structural formula I-AC:

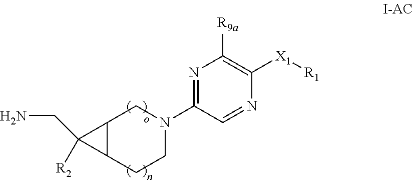

wherein n is 0 or 1, o is 1 or 2; R₉ₐ is H or NH₂; X₁, R₁ and R₂ are as defined above; preferably X₁ is a single bond or S.

In certain embodiments, compounds have formula I-AD:

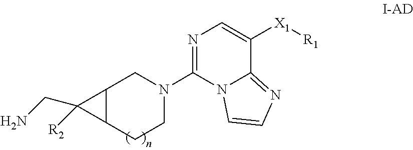

wherein n is 0 or 1; X₁, R₁ and R₂ are as defined above; preferably X₁ is a single bond or S.

In certain embodiments, compounds have formula I-AE or I-AE′:

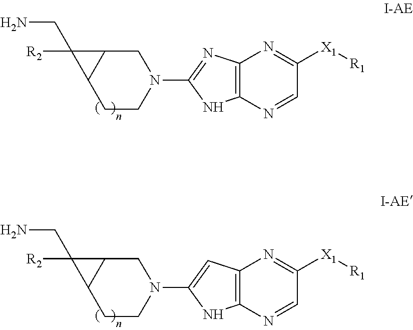

wherein n is 0 or 1; X₁, R₁ and R₂ are as defined above; preferably X₁ is a single bond or S.

In certain embodiments, compounds have formula I-AF or I-AF′:

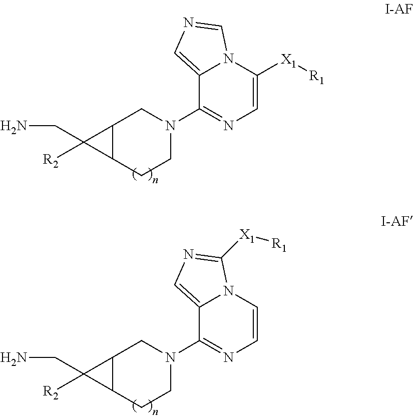

wherein n is 0 or 1; X₁, R₁ and R₂ are as defined above; preferably X₁ is a single bond or S.

In certain embodiments, compounds have formula I-AG or I-AG′ or I-AG″:

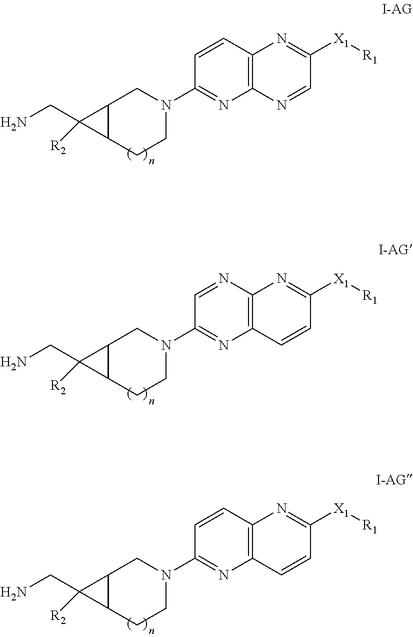

wherein n is 0 or 1; X₁, R₁ and R₂ are as defined above; preferably X₁ is S, O or a single bond.

In certain embodiments, compounds have formula I-AH:

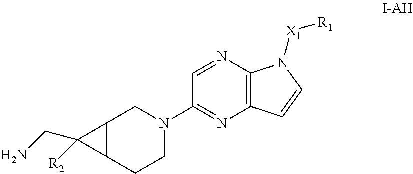

wherein n is 0 or 1; X₁, R₁ and R₂ are as defined above; preferably X₁ is a single bond.

In certain embodiments, compounds have formula I-AI:

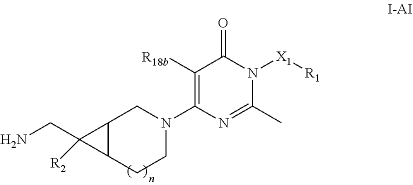

wherein n is 0 or 1; R_{18b} is H or halogen; X₁, R₁ and R₂ are as defined above; preferably X₁ is a single bond.

In certain embodiments, compounds have formula I-AJ:

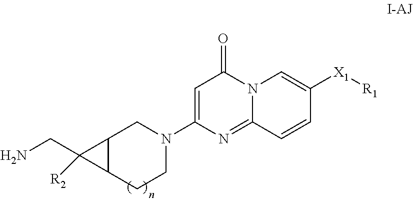

wherein n is 0 or 1; X₁, R₁ and R₂ are as defined above; preferably X₁ is a single bond or S.

In certain embodiments, compounds have formula I-AK:

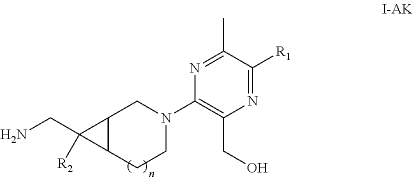

wherein n is 0 or 1; X₁, R₁ and R₂ are as defined above; preferably X₁ is a single bond or S.

In a preferred embodiment of the invention, the compounds are selected from the following list:
(3-(7-(aminomethyl)-7-(thiophen-3-yl)-3-azabicyclo[4.1.0]heptan-3-yl)-6-(2,3-dichlorophenyl)-5-methylpyrazin-2-yl)methanol
(3-(3-(2,3-dichlorophenyl)-1H-pyrazolo[3,4-b]pyrazin-6-yl)-7-(pyridin-3-yl)-3-azabicyclo[4.1.0]heptan-7-yl)methanamine
(3-(3-(2,3-dichlorophenyl)-1H-pyrazolo[3,4-b]pyrazin-6-yl)-7-(2-fluorophenyl)-3-azabicyclo[4.1.0]heptan-7-yl)methanamine
(3-(3-(benzo[b]thiophen-7-yl)-1H-pyrazolo[3,4-b]pyrazin-6-yl)-7-(2-fluorophenyl)-3-azabicyclo[4.1.0]heptan-7-yl)methanamine
(7-(2-fluorophenyl)-3-(3-(quinolin-5-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(3-methylisoxazol-5-yl)-3-azabicyclo[4.1.0]heptan-7-yl)methanamine
(3-(3-(2,3-dichlorophenyl)-1H-pyrazolo[3,4-b]pyrazin-6-yl)-7-(3-fluorophenyl)-3-azabicyclo[4.1.0]heptan-7-yl)methanamine
(3-(3-(2,3-dichlorophenyl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(quinolin-5-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(4-fluorophenyl)-3-azabicyclo[4.1.0]heptan-7-yl)methanamine
(7-(4-fluorophenyl)-3-(3-(quinoxalin-6-yl)-1H-pyrazolo[3,4-b]pyrazin-6-yl)-3-azabicyclo[4.1.0]heptan-7-yl)methanamine
(7-(4-fluorophenyl)-3-(3-(quinolin-8-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-1,2,3-triazol-4-yl)-3-azabicyclo[4.1.0]heptan-7-yl)methanamine
(3-(3-(2,3-dichlorophenyl)-1H-pyrazolo[3,4-b]pyrazin-6-yl)-7-(4-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(4-methylisoxazol-3-yl)-3-azabicyclo[4.1.0]heptan-7-yl)methanamine
4-(6-(7-(aminomethyl)-7-(2-fluorophenyl)-3-azabicyclo[4.1.0]heptan-3-yl)-1H-pyrazolo[3,4-b]pyrazin-3-yl)-3-chloro-N-methylpyridin-2-amine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)-3-chloro-N-methylpyridin-2-amine
(7-(5-methylisoxazol-3-yl)-3-(3-(thieno[3,2-b]pyridin-7-yl)-1H-pyrazolo[3,4-b]pyrazin-6-yl)-3-azabicyclo[4.1.0]heptan-7-yl)methanamine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)isoindolin-1-one
(3-(3-(benzo[d]thiazol-5-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(8-methylquinolin-5-yl)-1H-pyrazolo[3,4-b]pyrazin-6-yl)-3-azabicyclo[4.1.0]heptan-7-yl)methanamine
(3-(3-(8-methoxyquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(isoquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(2-methylquinolin-5-yl)-1H-pyrazolo[3,4-b]pyrazin-6-yl)-3-azabicyclo[4.1.0]heptan-7-yl)methanamine
(3-(3-(2-methyl-2H-indazol-4-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(quinolin-4-yl)-1H-pyrazolo[3,4-b]pyrazin-6-yl)-3-azabicyclo[4.1.0]heptan-7-yl)methanamine
(3-(3-(2-methoxypyridin-3-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-((S)-6a,7,8,9-tetrahydro-6H-pyrido[3,2-b]pyrrolo[1,2-d][1,4]oxazin-4-yl)-1H-pyrazolo[3,4-b]pyrazin-6-yl)-3-azabicyclo[4.1.0]heptan-7-yl)methanamine
(3-(3-(8-fluoroquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(2,3-dichloropyridin-4-yl)-1H-pyrazolo[3,4-b]pyrazin-6-yl)-7-(5-methylisoxazol-3-yl)-3-azabicyclo[4.1.0]heptan-7-yl)methanamine
5-(6-(7-(aminomethyl)-7-(5-methylisoxazol-3-yl)-3-azabicyclo[4.1.0]heptan-3-yl)-1H-pyrazolo[3,4-b]pyrazin-3-yl)quinoline-8-carbonitrile
(3-(3-(8-chloroquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(8-chloroisoquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(1,5-naphthyridin-4-yl)-1H-pyrazolo[3,4-b]pyrazin-6-yl)-7-(5-methylisoxazol-3-yl)-3-azabicyclo[4.1.0]heptan-7-yl)methanamine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)-3-chloropyridin-2-amine
((1S,6R,7S)-3-(3-(2,2-difluorobenzo[d][1,3]dioxol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(1,8-naphthyridin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2,3-dihydrobenzo[b][1,4]dioxin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(5-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-chloroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(8-(trifluoromethyl)quinolin-5-yl)-1H-pyrazolo[3,4-b]pyrazin-6-yl)-3-azabicyclo[4.1.0]heptan-7-yl)methanamine
((1S,6R,7S)-3-(3-(3,4-dichloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(7-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-(methoxymethyl)isoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-cyclopropylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(3-fluoropyridin-2-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-fluoropyridin-2-yl)-3-azabicyclo[4.1.0]heptan-7-yl)methanamine
4-(6-((1S,6R,7S)-7-(aminomethyl)-7-(5-methylisoxazol-3-yl)-3-azabicyclo[4.1.0]heptan-3-yl)-1H-pyrazolo[3,4-b]pyrazin-3-yl)-3,3-difluoro-1-methylindolin-2-one
(3-(3-(3-methylbenzo[d]isoxazol-5-yl)-1H-pyrazolo[3,4-b]pyrazin-6-yl)-7-(4-methylisoxazol-3-yl)-3-azabicyclo[4.1.0]heptan-7-yl)methanamine
4-((6-(7-(aminomethyl)-7-(5-methylisoxazol-3-yl)-3-azabicyclo[4.1.0]heptan-3-yl)-1H-pyrazolo[3,4-b]pyrazin-3-yl)thio)-3-chloropyridin-2-amine
(7-(5-methylisoxazol-3-yl)-3-(3-(thieno[3,2-b]pyridin-7-ylthio)-1H-pyrazolo[3,4-b]pyrazin-6-yl)-3-azabicyclo[4.1.0]heptan-7-yl)methanamine
(6-(7-(aminomethyl)-7-(3-fluorophenyl)-3-azabicyclo[4.1.0]heptan-3-yl)-3-(2,3-dichlorophenyl)-1H-pyrazolo[3,4-b]pyrazin-5-yl)methanol
(6-(7-(aminomethyl)-7-(3-fluorophenyl)-3-azabicyclo[4.1.0]heptan-3-yl)-3-phenyl-1H-pyrazolo[3,4-b]pyrazin-5-yl)methanol
(3-((2-amino-3-chloropyridin-4-yl)thio)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol
(3-(2-amino-3-chloropyridin-4-yl)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol
4-((6-(7-(aminomethyl)-7-(2-fluorophenyl)-3-azabicyclo[4.1.0]heptan-3-yl)-5H-pyrrolo[2,3-b]pyrazin-2-yl)thio)-3-chloropyridin-2-amine
(3-(3-(3,4-dihydro-1,5-naphthyridin-1(2H)-yl)-1H-pyrazolo[3,4-b]pyrazin-6-yl)-7-(5-methylisoxazol-3-yl)-3-azabicyclo[4.1.0]heptan-7-yl)methanamine
(3-(7-(aminomethyl)-3-(3-(4-chloro-2-methyl-2H-indazol-5-yl)-1H-pyrazolo[3,4-b]pyrazin-6-yl)-3-azabicyclo[4.1.0]heptan-7-yl)isoxazol-5-yl)methanol
6-(7-(aminomethyl)-7-(5-methylisoxazol-3-yl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one
(7-phenyl-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
4-((2-(7-(aminomethyl)-7-phenyl-3-azabicyclo[4.1.0]heptan-3-yl)-1H-imidazo[4,5-b]pyrazin-5-yl)thio)-3-chloropyridin-2-amine
4-((2-(7-(aminomethyl)-7-(4-methylthiazol-2-yl)-3-azabicyclo[4.1.0]heptan-3-yl)-1H-imidazo[4,5-b]pyrazin-5-yl)thio)-3-chloropyridin-2-amine
6-(7-(aminomethyl)-7-phenyl-3-azabicyclo[4.1.0]heptan-3-yl)-3-((2-(trifluoromethyl)pyridin-3-yl)thio)pyrazin-2-amine
6-(7-(aminomethyl)-7-(pyridin-2-yl)-3-azabicyclo[4.1.0]heptan-3-yl)-3-((2-(trifluoromethyl)pyridin-3-yl)thio)pyrazin-2-amine
6-(7-(aminomethyl)-7-(4-methylthiazol-2-yl)-3-azabicyclo[4.1.0]heptan-3-yl)-3-((2-(trifluoromethyl)pyridin-3-yl)thio)pyrazin-2-amine
3-((2-amino-3-chloropyridin-4-yl)thio)-6-(7-(aminomethyl)-7-(4-methylthiazol-2-yl)-3-azabicyclo[4.1.0]heptan-3-yl)pyrazin-2-amine
4-((5-(7-(aminomethyl)-7-(4-methylthiazol-2-yl)-3-azabicyclo[4.1.0]heptan-3-yl)imidazo[1,2-c]pyrimidin-8-yl)thio)-3-chloropyridin-2-amine
(3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine
(3-(3-(2,3-dichlorophenyl)-1H-pyrazolo[3,4-b]pyrazin-6-yl)-7-phenyl-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-phenyl-3-azabicyclo[4.1.0]heptan-7-yl)methanamine
(3-(3-(2,3-dichlorophenyl)-1H-pyrazolo[3,4-b]pyrazin-6-yl)-7-(pyridin-2-yl)-3-azabicyclo[4.1.0]heptan-7-yl)methanamine
(3-(3-(2,3-dichlorophenyl)-1H-pyrazolo[3,4-b]pyrazin-6-yl)-7-(pyrimidin-2-yl)-3-azabicyclo[4.1.0]heptan-7-yl)methanamine
(3-(3-(2-chlorophenyl)-1H-pyrazolo[3,4-b]pyrazin-6-yl)-7-(4-methylthiazol-2-yl)-3-azabicyclo[4.1.0]heptan-7-yl)methanamine
(3-(3-(3-chlorophenyl)-1H-pyrazolo[3,4-b]pyrazin-6-yl)-7-(4-methylthiazol-2-yl)-3-azabicyclo[4.1.0]heptan-7-yl)methanamine
(3-(3-(2,3-difluorophenyl)-1H-pyrazolo[3,4-b]pyrazin-6-yl)-7-(4-methylthiazol-2-yl)-3-azabicyclo[4.1.0]heptan-7-yl)methanamine
(3-(3-(2-chloro-3-methoxyphenyl)-1H-pyrazolo[3,4-b]pyrazin-6-yl)-7-(4-methylthiazol-2-yl)-3-azabicyclo[4.1.0]heptan-7-yl)methanamine
(7-(4-methylthiazol-2-yl)-3-(3-(quinolin-5-yl)-1H-pyrazolo[3,4-b]pyrazin-6-yl)-3-azabicyclo[4.1.0]heptan-7-yl)methanamine
(7-(4-methylthiazol-2-yl)-3-(3-(naphthalen-1-yl)-1H-pyrazolo[3,4-b]pyrazin-6-yl)-3-azabicyclo[4.1.0]heptan-7-yl)methanamine
(7-(4-methylthiazol-2-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine
(3-(3-(benzo[d][1,3]dioxol-4-yl)-1H-pyrazolo[3,4-b]pyrazin-6-yl)-7-(4-methylthiazol-2-yl)-3-azabicyclo[4.1.0]heptan-7-yl)methanamine
(3-(3-(8-fluoroquinolin-5-yl)-1H-pyrazolo[3,4-b]pyrazin-6-yl)-7-(4-methylthiazol-2-yl)-3-azabicyclo[4.1.0]heptan-7-yl)methanamine
4-((6-(7-(aminomethyl)-7-(4-methylthiazol-2-yl)-3-azabicyclo[4.1.0]heptan-3-yl)-1H-pyrazolo[3,4-b]pyrazin-3-yl)thio)-3-chloropyridin-2-amine
(3-(3-(2,3-dichlorophenyl)-1H-pyrazolo[3,4-b]pyrazin-6-yl)-7-(thiophen-3-yl)-3-azabicyclo[4.1.0]heptan-7-yl)methanamine
(7-(thiophen-3-yl)-3-(3-(2-(trifluoromethyl)pyridin-4-yl)-1H-pyrazolo[3,4-b]pyrazin-6-yl)-3-azabicyclo[4.1.0]heptan-7-yl)methanamine
(7-(thiophen-3-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-pyrazol-5-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-pyrazol-3-yl)-3-azabicyclo[4.1.0]heptan-7-yl)methanamine
(3-(3-(2,3-dichlorophenyl)-1H-pyrazolo[3,4-b]pyrazin-6-yl)-7-(isoxazol-3-yl)-3-azabicyclo[4.1.0]heptan-7-yl)methanamine
(6-(7-(aminomethyl)-7-(4-methylthiazol-2-yl)-3-azabicyclo[4.1.0]heptan-3-yl)-3-(quinolin-5-yl)-1H-pyrazolo[3,4-b]pyrazin-5-yl)methanol
(6-(7-(aminomethyl)-7-(4-methylthiazol-2-yl)-3-azabicyclo[4.1.0]heptan-3-yl)-3-(2,3-dichlorophenyl)-1H-pyrazolo[3,4-b]pyrazin-5-yl)methanol
4-(6-(7-(aminomethyl)-7-(4-methylthiazol-2-yl)-3-azabicyclo[4.1.0]heptan-3-yl)-1H-pyrazolo[3,4-b]pyrazin-3-yl)naphthalen-1-yl sulfurofluoridate
6-((1R,5S,6s)-6-(aminomethyl)-6-phenyl-3-azabicyclo[3.1.0]hexan-3-yl)-3-((2-(trifluoromethyl)pyridin-3-yl)thio)pyrazin-2-amine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-amine
((1S,6R,7R)-7-(2,6-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-2,3-dimethyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-3-methoxy-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(7-chloro-2-methylbenzo[d]thiazol-6-yl)-5-methyl-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2-chloro-3-(oxazol-2-yl)phenyl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(5-chloro-3-methoxyquinoxalin-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(thieno[2,3-b]pyridin-3-yl)-1H-pyrazolo[3,4-b]pyrazin-6-yl)-3-azabicyclo[4.1.0]heptan-7-yl)methanamine
((1S,6R,7S)-3-(3-(8-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-methoxyquinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7R)-7-(2,6-difluorophenyl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(2,6-difluorophenyl)-3-azabicyclo[4.1.0]heptan-7-yl)methanamine
((1S,6R,7R)-3-(3-(benzo[d]thiazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(5-chloro-3-methoxyquinoxalin-6-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(5-fluoropyridin-2-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(5-fluoropyridin-2-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
((1S,6R,7S)-3-(3-(4-chloro-2-methyl-2H-indazol-5-yl)-1H-pyrazolo[3,4-b]pyrazin-6-yl)-7-(isoxazol-3-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(isoxazol-3-yl)-3-azabicyclo[4.1.0]heptan-7-yl)methanamine
((1S,6R,7S)-3-(3-(8-methoxyquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(8-(trifluoromethoxy)quinolin-5-yl)-1H-pyrazolo[3,4-b]pyrazin-6-yl)-3-azabicyclo[4.1.0]heptan-7-yl)methanamine
((1S,6R,7S)-3-(3-(2,4-dimethyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(8-(methoxymethyl)quinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7S)-3-(3-(7-fluoroquinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7R)-7-(2-fluorophenyl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-fluoroquinolin-4-yl)-1H-pyrazolo[3,4-b]pyrazin-6-yl)-3-azabicyclo[4.1.0]heptan-7-yl)methanamine
((1S,6R,7S)-3-(3-(8-(difluoromethyl)quinolin-5-yl)-1H-pyrazolo[3,4-b]pyrazin-6-yl)-7-(5-methylisoxazol-3-yl)-3-azabicyclo[4.1.0]heptan-7-yl)methanamine
1-(5-(6-((1S,6R,7S)-7-(aminomethyl)-7-(5-methylisoxazol-3-yl)-3-azabicyclo[4.1.0]heptan-3-yl)-1H-pyrazolo[3,4-b]pyrazin-3-yl)quinolin-8-yl)ethan-1-ol
4-((8-((1S,6R,7R)-7-(aminomethyl)-7-(2-fluorophenyl)-3-azabicyclo[4.1.0]heptan-3-yl)imidazo[1,5-a]pyrazin-5-yl)thio)-3-chloropyridin-2-amine
((1S,6R,7R)-7-(2-fluorophenyl)-3-(3-(pyrazolo[1,5-a]pyridin-5-yl)-1H-pyrazolo[3,4-b]pyrazin-6-yl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)propan-2-ol
((1S,6R,7R)-7-(2-fluorophenyl)-3-(3-(8-isopropoxyquinolin-5-yl)-1H-pyrazolo[3,4-b]pyrazin-6-yl)-3-azabicyclo[4.1.0]heptan-7-yl)methanamine
((1S,6R,7R)-3-(3-(8-cyclopropylquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7S)-7-(3-fluoropyridin-2-yl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(quinolin-6-yl)-1H-pyrazolo[3,4-b]pyrazin-6-yl)-3-azabicyclo[4.1.0]heptan-7-yl)methanamine
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)ethan-1-ol
((1S,6R,7R)-7-(2-fluorophenyl)-3-(3-(imidazo[1,2-a]pyridin-5-yl)-1H-pyrazolo[3,4-b]pyrazin-6-yl)-3-azabicyclo[4.1.0]heptan-7-yl)methanamine
((1S,6R,7S)-7-(3-fluoropyridin-2-yl)-3-(3-(8-(trifluoromethoxy)quinolin-5-yl)-1H-pyrazolo[3,4-b]pyrazin-6-yl)-3-azabicyclo[4.1.0]heptan-7-yl)methanamine
((1S,6R,7R)-3-(3-(2,4-dimethyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(6-methoxypyrazolo[1,5-a]pyridin-4-yl)-1H-pyrazolo[3,4-b]pyrazin-6-yl)-3-azabicyclo[4.1.0]heptan-7-yl)methanamine
((1S,6R,7S)-3-(3-(1,7-naphthyridin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2-methoxyquinolin-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(2-(trifluoromethyl)quinolin-6-yl)-1H-pyrazolo[3,4-b]pyrazin-6-yl)-3-azabicyclo[4.1.0]heptan-7-yl)methanamine
((1S,6R,7S)-3-(3-(7-methoxyquinolin-3-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(7-(trifluoromethyl)quinolin-3-yl)-1H-pyrazolo[3,4-b]pyrazin-6-yl)-3-azabicyclo[4.1.0]heptan-7-yl)methanamine
((1S,6R,7R)-3-(3-(1,6-naphthyridin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2,3-dihydrofuro[2,3-b]pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1,7-naphthyridin-4-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methanamine
((1S,6R,7R)-3-(7-(2,4-dimethyl-2H-indazol-5-yl)-5H-pyrrolo[2,3-b]pyrazin-3-yl)-7-(2-fluorophenyl)-3-azabicyclo[4.1.0]heptan-7-yl)methanamine
((1S,6R,7R)-3-(3-(2-chloro-3-(pyrazin-2-yl)phenyl)-1H-pyrazolo[3,4-b]pyrazin-6-yl)-7-(2-fluorophenyl)-3-azabicyclo[4.1.0]heptan-7-yl)methanamine
(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)methanol
((1S,6R,7R)-3-(3-(8-(difluoromethoxy)quinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(7-methylpyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-3-(3-(3-bromo-2-chlorophenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)pyrimidin-2-amine
((1S,6R,7R)-3-(3-(8-ethoxyquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(8-(1-methoxyethyl)quinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methoxyquinolin-4-yl)-1H-pyrazolo[3,4-b]pyrazin-6-yl)-3-azabicyclo[4.1.0]heptan-7-yl)methanamine
((1S,6R,7R)-3-(3-(5-chloroquinoxalin-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-3-(oxazol-2-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3,4-dihydro-1,5-naphthyridin-1(2H)-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-chloro-2-(oxazol-2-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(7-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(5-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-fluoroquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(7-fluoroquinolin-6-yl)-1H-pyrazolo[3,4-b]pyrazin-6-yl)-3-azabicyclo[4.1.0]heptan-7-yl)methanamine
((1S,6R,7R)-3-(3-(3-chloropyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2,3-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-3-chloropyridin-2-amine
((1S,6R,7S)-3-(3-(2,4-dimethyl-2H-indazol-5-yl)-1H-pyrazolo[3,4-b]pyrazin-6-yl)-7-(3-fluoropyridin-2-yl)-3-azabicyclo[4.1.0]heptan-7-yl)methanamine
((1S,6R,7R)-7-(2-chlorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-chlorophenyl)-3-azabicyclo[4.1.0]heptan-3-yl)-1H-pyrazolo[3,4-b]pyrazin-3-yl)-3-chloropyridin-2-amine
((1S,6R,7R)-3-(3-(2,4-dimethyl-2H-indazol-5-yl)-1H-pyrazolo[3,4-b]pyrazin-6-yl)-7-(2-(trifluoromethyl)phenyl)-3-azabicyclo[4.1.0]heptan-7-yl)methanamine
((1S,6R,7R)-7-(2,3-difluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-3-(3-(2,4-dimethyl-2H-indazol-5-yl)-1H-pyrazolo[3,4-b]pyrazin-6-yl)-7-(2-fluoro-3-methylphenyl)-3-azabicyclo[4.1.0]heptan-7-yl)methanamine
((1S,6R,7S)-7-(5-cyclopropyl-3-fluoropyridin-2-yl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(furo[2,3-b]pyridin-4-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
((1S,6R,7R)-7-(2-fluorophenyl)-3-(3-(3-methoxyquinoxalin-6-yl)-1H-pyrazolo[3,4-b]pyrazin-6-yl)-3-azabicyclo[4.1.0]heptan-7-yl)methanamine
((1S,6R,7R)-3-(3-(3-chloro-2-methylpyridin-4-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N,N-dimethylpyridin-2-amine
((1S,6R,7R)-7-(2-fluorophenyl)-3-(3-(7-methoxyquinolin-4-yl)-1H-pyrazolo[3,4-b]pyrazin-6-yl)-3-azabicyclo[4.1.0]heptan-7-yl)methanamine
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)-N,N-dimethylmethanamine
((1S,6R,7R)-7-(2-fluorophenyl)-3-(3-(4-methylbenzo[c][1,2,5]oxadiazol-5-yl)-1H-pyrazolo[3,4-b]pyrazin-6-yl)-3-azabicyclo[4.1.0]heptan-7-yl)methanamine
((1S,6R,7R)-3-(3-(4-cyclopropyl-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-fluoro-2-methylpyridin-4-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloropyridin-2-amine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyclopropylpyridin-2-amine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylpyridin-2(1H)-one
((1S,6R,7R)-7-(2,4-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2,4-difluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methoxy-2-methylquinolin-5-yl)-1H-pyrazolo[3,4-b]pyrazin-6-yl)-3-azabicyclo[4.1.0]heptan-7-yl)methanamine
((1S,6R,7R)-3-(3-(4-(difluoromethyl)-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1,4-dimethyl-1H-benzo[d][1,2,3]triazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2,4-dimethyl-2H-benzo[d][1,2,3]triazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,3-dimethylindolin-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-2H-benzo[b][1,4]oxazin-3(4H)-one
((1S,6R,7R)-3-(3-(4-chloro-2-ethyl-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
((1S,6R,7R)-7-(2-fluorophenyl)-3-(3-(8-methyl-[1,2,4]triazolo[4,3-a]pyridin-7-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N,N-dimethylquinoline-8-carboxamide
((1R,5S,6r)-3-(3-(8-fluorodecahydroquinolin-5-yl)-4,7-dihydro-1H-pyrazolo[3,4-b]pyrazin-6-yl)-6-(4-methylthiazol-2-yl)-3-azabicyclo[3.1.0]hexan-6-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methoxypyridin-2-amine
((1S,6R,7R)-3-(3-(2,3-dichloro-4-((1-methyl-1H-pyrazol-3-yl)oxy)phenyl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloroindolin-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylindolin-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoindolin-1-one
((1S,6R,7R)-7-(2-fluorophenyl)-3-(3-(8-methoxyisoquinolin-5-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinoline-8-carboxamide
((1R,5S,6s)-3-(3-(4-chloro-2-methyl-2H-indazol-5-yl)-1H-pyrazolo[3,4-b]pyrazin-6-yl)-6-(2,6-difluorophenyl)-3-azabicyclo[3.1.0]hexan-6-yl)methanamine
((1S,6R,7R)-7-(2-fluorophenyl)-3-(3-(5-methylquinoxalin-6-yl)-1H-pyrazolo[3,4-b]pyrazin-6-yl)-3-azabicyclo[4.1.0]heptan-7-yl)methanamine
methyl 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzoate
((1S,6R,7R)-7-(2-fluorophenyl)-3-(3-(imidazo[1,2-a]pyridin-3-yl)-1H-pyrazolo[3,4-b]pyrazin-6-yl)-3-azabicyclo[4.1.0]heptan-7-yl)methanamine
((1S,6R,7R)-3-(3-(7-fluoro-8-methoxyquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(8-fluoro-2-methylquinolin-5-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-dichlorobenzoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-4-methyl-2H-benzo[b][1,4]oxazin-3(4H)-one
((1S,6R,7R)-3-(3-(4-chloro-2-(oxetan-3-yl)-2H-indazol-5-yl)-1H-pyrazolo[3,4-b]pyrazin-6-yl)-7-(2-fluorophenyl)-3-azabicyclo[4.1.0]heptan-7-yl)methanamine
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dimethyl-1,3-dihydro-2H-benzo[d]imidazol-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-fluoroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-chloroquinolin-2(1H)-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dihydro-2H-benzo[d]imidazol-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-dichlorobenzamide
((1S,6R,7R)-7-(2-fluorophenyl)-3-(3-(quinolin-5-yl)-1H-pyrazolo[3,4-b]pyrazin-6-yl)-3-azabicyclo[4.1.0]heptan-7-yl)methanamine
((1S,6R,7R)-3-(3-(4-chloro-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylisoindolin-1-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzonitrile
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylbenzamide
((1S,6R,7R)-7-(2-fluorophenyl)-3-(3-(isoquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(isoquinolin-7-yl)-1H-pyrazolo[3,4-b]pyrazin-6-yl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)benzo[d]isothiazol-3(2H)-one 1,1-dioxide
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzamide
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroisoquinolin-1(2H)-one
((1S,6R,7R)-7-(2-fluorophenyl)-3-(3-(quinolin-7-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-chloro-5-fluorobenzamide
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)phthalazin-1(2H)-one
((1S,6R,7R)-3-(3-(3-chloroquinolin-7-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-naphthoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-4-amine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methyl-1,3-dihydro-2H-benzo[d]imidazol-2-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4H-pyrido[1,2-a]pyrimidin-4-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3,4-trimethyl-1,3-dihydro-2H-benzo[d]imidazol-2-one
((1S,6R,7R)-7-(2-fluorophenyl)-3-(3-(3-methoxycinnolin-7-yl)-1H-pyrazolo[3,4-b]pyrazin-6-yl)-3-azabicyclo[4.1.0]heptan-7-yl)methanamine
((1S,6R,7R)-3-(3-(3-chloro-2-(oxazol-5-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(oxazol-2-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-chloro-2-(1-methyl-1H-pyrazol-3-yl)pyridin-4-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)quinoline-8-carboxylic acid
5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-N-methylquinoline-8-carboxamide
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2(1H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one
((1S,6R,7R)-7-(2-fluorophenyl)-3-(3-(spiro[cyclobutane-1,3′-pyrrolo[3,2-b]pyridin]-1′(2′H)-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzoic acid
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylquinolin-2(1H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-methylbenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylisoquinolin-1(2H)-one
((1R,5S,6r)-3-(3-(4-chloro-2-methyl-2H-indazol-5-yl)-1H-pyrazolo[3,4-b]pyrazin-6-yl)-6-(5-methylisoxazol-3-yl)-3-azabicyclo[3.1.0]hexan-6-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzamide
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-cyclopropylquinoline-8-carboxamide
((1S,6R,7R)-3-(3-(4-chloro-2-isopropyl-2H-indazol-5-yl)-1H-pyrazolo[3,4-b]pyrazin-6-yl)-7-(2-fluorophenyl)-3-azabicyclo[4.1.0]heptan-7-yl)methanamine
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-(methylsulfonyl)quinoline-8-carboxamide
((1R,5S,6r)-3-(3-(2-chloro-4-(1H-pyrazol-1-yl)phenyl)-1H-pyrazolo[3,4-b]pyrazin-6-yl)-6-(4-methylthiazol-2-yl)-3-azabicyclo[3.1.0]hexan-6-yl)methanamine
((1S,6R,7R)-3-(3-(3-chloro-2-(pyrimidin-5-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(2H-tetrazol-5-yl)phenyl)-1H-pyrazolo[3,4-b]pyrazin-6-yl)-7-(2-fluorophenyl)-3-azabicyclo[4.1.0]heptan-7-yl)methanamine
7-(aminomethyl)-3-(3-(4-chloro-2-methyl-2H-indazol-5-yl)-1H-pyrazolo[3,4-b]pyrazin-6-yl)-N,N-dimethyl-3-azabicyclo[4.1.0]heptane-7-carboxamide
7-(aminomethyl)-3-(3-(4-chloro-2-methyl-2H-indazol-5-yl)-1H-pyrazolo[3,4-b]pyrazin-6-yl)-N-methyl-3-azabicyclo[4.1.0]heptane-7-carboxamide
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,4,5,6-tetrahydro-7H-pyrrolo[3,4-b]pyridin-7-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methyl-1,3-dihydro-2H-benzo[d]imidazol-2-one
((1S,6R,7R)-7-(2-fluorophenyl)-3-(3-(quinazolin-6-yl)-1H-pyrazolo[3,4-b]pyrazin-6-yl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(3,5-difluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methoxy-1,3-dihydro-2H-benzo[d]imidazol-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-oxadiazol-5-ol
((1S,6R,7R)-3-(3-(2-chloro-4-(1H-pyrazol-5-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(1-methyl-1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroisoquinolin-1(2H)-one
6-(6-((1S,6R,7R)-7-(aminomethyl)-7-(2-fluorophenyl)-3-azabicyclo[4.1.0]heptan-3-yl)-4H-114-pyrazolo[4,5-b]pyrazin-3-yl)-5-fluoroisoquinolin-1(2H)-one
6-(6-((1S,6R,7R)-7-(aminomethyl)-7-(2-fluorophenyl)-3-azabicyclo[4.1.0]heptan-3-yl)-114,212-pyrazolo[3,4-b]pyrazin-3-yl)-2-methylisoquinolin-1(2H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinazolin-4(3H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1,3-dihydro-2H-benzo[d]imidazol-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dihydro-2H-pyrrolo[2,3-b]pyridin-2-one
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-1-yl)ethan-1-one
((1S,6R,7R)-7-(2-fluorophenyl)-3-(3-(1-methyl-1H-benzo[d]imidazol-6-yl)-1H-pyrazolo[3,4-b]pyrazin-6-yl)-3-azabicyclo[4.1.0]heptan-7-yl)methanamine
6-(6-((1S,6R,7R)-7-(aminomethyl)-7-(3,5-difluorophenyl)-3-azabicyclo[4.1.0]heptan-3-yl)-1H-pyrazolo[3,4-b]pyrazin-3-yl)-5-fluoroisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methylpicolinamide
((1S,6R,7R)-7-(3,5-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(2-(trifluoromethyl)quinazolin-6-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyanobenzamide
((1S,6R,7R)-3-(3-(5-chloro-3,4-dihydro-2H-benzo[b][1,4]oxazin-6-yl)-1,7-dihydro-412-pyrazolo[4,3-b]pyrazin-6-yl)-7-(2-fluorophenyl)-3-azabicyclo[4.1.0]heptan-7-yl)methanamine
((1S,6R,7R)-7-(2-fluorophenyl)-3-(3-(indolin-5-yl)-1H-pyrazolo[3,4-b]pyrazin-6-yl)-3-azabicyclo[4.1.0]heptan-7-yl)methanamine
4′-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3′-chloro-3,5-difluoro-2,3,4,5-tetrahydro-[1,1′-biphenyl]-4-ol
5-(6-((1S,6R,7R)-7-(aminomethyl)-7-(2-fluorophenyl)-3-azabicyclo[4.1.0]heptan-3-yl)-4,7-dihydro-1H-pyrazolo[3,4-b]pyrazin-3-yl)-8-fluoroisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-fluoro-2-methylisoquinolin-1(2H)-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-fluoro-2-methylisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1,3-dimethyl-1,3-dihydro-2H-benzo[d]imidazol-2-one
((1S,6R,7R)-3-(3-(1,4-dimethyl-4,7-dihydro-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-2,4-dihydro-1H-pyrazolo[3,4-b]pyrazin-3-yl)isoquinolin-1-amine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methyl-1,3-dihydro-2H-benzo[d]imidazol-2-one
((1S,6R,7R)-3-(3-(5-chloro-4-methyl-3,4-dihydro-2H-benzo[b][1,4]oxazin-6-yl)-2,3,4,7-tetrahydro-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenol
((1S,6R,7R)-3-(3-(3-chloro-3′,5′-difluoro-[1,1′-biphenyl]-4-yl)-1H-pyrazolo[3,4-b]pyrazin-6-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2H-indazol-2-yl)acetamide
((1S,6R,7R)-3-(3-(4-chloro-1-isopropyl-1H-indazol-5-yl)-1H-pyrazolo[3,4-b]pyrazin-6-yl)-7-(2-fluorophenyl)-3-azabicyclo[4.1.0]heptan-7-yl)methanamine
1-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)pyrrolidin-2-one
((1S,6R,7R)-3-(3-([1,2,4]triazolo[1,5-a]pyridin-6-yl)-1H-pyrazolo[3,4-b]pyrazin-6-yl)-7-(2-fluorophenyl)-3-azabicyclo[4.1.0]heptan-7-yl)methanamine
2-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)acetamide
((1S,6R,7R)-3-(3-(4-chloro-1-methyl-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3,5-difluorophenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-fluorophenol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-fluorophenol
((1S,6R,7R)-3-(3-([1,2,4]triazolo[1,5-a]pyridin-7-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-((2-fluorophenyl)ethynyl)-1H-pyrazolo[3,4-b]pyrazin-6-yl)-3-azabicyclo[4.1.0]heptan-7-yl)methanamine
((1S,6R,7R)-3-(3-(4-chloro-2-(difluoromethyl)-2H-indazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-7H-114-pyrazolo[4,5-b]pyrazin-3-yl)-5-chloro-2-methylisoquinolin-1(2H)-one
((1S,6R,7R)-7-(2-fluorophenyl)-3-(3-(2-methyl-1H-benzo[d]imidazol-5-yl)-1H-pyrazolo[3,4-b]pyrazin-6-yl)-3-azabicyclo[4.1.0]heptan-7-yl)methanamine
((1S,6R,7R)-3-(3-(7-chloro-1-methyl-1H-benzo[d]imidazol-6-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-2-methylphthalazin-1(2H)-one
6-(6-((1S,6R,7R)-7-(aminomethyl)-7-(2-fluorophenyl)-3-azabicyclo[4.1.0]heptan-3-yl)-114,212-pyrazolo[3,4-b]pyrazin-3-yl)-5-chlorophthalazin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzo[d]oxazol-2(3H)-one
((1S,6R,7R)-3-(5-((3-chloro-2-(1H-pyrazol-1-yl)pyridin-4-yl)thio)-1H-imidazo[4,5-b]pyrazin-2-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-5-methyl-4-oxo-4,5-dihydro-1H-pyrazolo[3,4-d]pyrimidin-3-yl)quinoline-8-carboxamide
6-((1S,6R,7R)-7-(aminomethyl)-7-(3,5-difluorophenyl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-(trifluoromethyl)pyridin-2-ol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzenesulfonamide
((1S,6R,7R)-7-(2-fluorophenyl)-3-(3-(1-methyl-1H-pyrazol-4-yl)-1H-pyrazolo[3,4-b]pyrazin-6-yl)-3-azabicyclo[4.1.0]heptan-7-yl)methanamine
((1S,6R,7R)-3-(3-(1,5-dimethyl-1H-pyrazol-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(4-chloro-1,2-dimethyl-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2-methylisoindolin-1-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-fluoro-2-(2-hydroxyethyl)isoquinolin-1(2H)-one
((1S,6R,7R)-7-(2-fluorophenyl)-3-(3-(1-methoxyisoquinolin-6-yl)-2,4-dihydro-1H-pyrazolo[3,4-b]pyrazin-6-yl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzo[d]oxazol-2(3H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,5-difluorophenol
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyano-N-methylbenzamide
6-(6-((1S,6R,7R)-7-(aminomethyl)-7-(2-fluorophenyl)-3-azabicyclo[4.1.0]heptan-3-yl)-114,712-pyrazolo[5,4-b]pyrazin-3-yl)-5-fluoro-2-(2-methoxyethyl)isoquinolin-1(2H)-one
((1S,6R,7R)-3-(3-(4-chloro-2,3-dimethyl-2,3-dihydro-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenyl acetate
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-(trifluoromethyl)phenol
((1S,6R,7R)-3-(3-(4-chloro-2-(2,2,2-trifluoroethyl)-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-3,3-difluoro-1-methylindolin-2-one
((1S,6R,7R)-3-(3-(7-chloro-2-methyl-2H-indazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(7-chloro-1-methyl-1H-indazol-6-yl)-1H-pyrazolo[3,4-b]pyrazin-6-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-2,4-dihydro-1H-pyrazolo[3,4-b]pyrazin-3-yl)-4-chloro-2H-indazol-2-yl)ethan-1-ol
((1S,6R,7R)-3-(3-(3,3-dimethylbut-1-yn-1-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-fluorobenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-methylpyridin-2-amine
((1S,6R,7R)-3-(3-(4-chloro-2-methyl-2H-indazol-5-yl)-1H-pyrazolo[3,4-b]pyrazin-6-yl)-7-(2,5-difluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-7-chloroisoquinolin-1(2H)-one
((1S,6R,7R)-7-(2-fluorophenyl)-3-(3-(1,3,5-trimethyl-1H-pyrazol-4-yl)-1H-pyrazolo[3,4-b]pyrazin-6-yl)-3-azabicyclo[4.1.0]heptan-7-yl)methanamine
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinazolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-3,3-difluoroindolin-2-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-difluorophenol
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-chloroindoline-2,3-dione
((1S,6R,7R)-7-(2-fluorophenyl)-3-(3-(8-fluoroquinolin-5-yl)-1H-pyrazolo[3,4-b]pyrazin-6-yl)-3-azabicyclo[4.1.0]heptan-7-yl)methanamine
((1S,6R,7R)-3-(3-(2-chloro-4-(5-methyl-1,2,4-oxadiazol-3-yl)phenyl)-1H-pyrazolo[3,4-b]pyrazin-6-yl)-7-(2-fluorophenyl)-3-azabicyclo[4.1.0]heptan-7-yl)methanamine
2-((1S,6R,7R)-7-(aminomethyl)-7-(2-fluorophenyl)-3-azabicyclo[4.1.0]heptan-3-yl)-5-(4-chloro-2-methyl-1H-214-indazol-5-yl)-3-methyl-3,7-dihydro-4H-pyrrolo[2,3-d]pyrimidin-4-one
3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-thiadiazol-5-ol
6-(2-((1S,6R,7R)-7-(aminomethyl)-7-(2-fluorophenyl)-3-azabicyclo[4.1.0]heptan-3-yl)-3-methyl-4-oxo-4,7-dihydro-3H-pyrrolo[2,3-d]pyrimidin-5-yl)-5-fluoroisoquinolin-1(2H)-one
(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenyl)methanol
((1S,6R,7R)-7-(2-fluorophenyl)-3-(5-(quinolin-4-yl)-5H-pyrrolo[2,3-b]pyrazin-2-yl)-3-azabicyclo[4.1.0]heptan-7-yl)methanamine
N-(((1S,6R,7R)-7-(2-fluorophenyl)-3-(3-(2-methyl-2H-indazol-5-yl)-1H-pyrazolo[3,4-b]pyrazin-6-yl)-3-azabicyclo[4.1.0]heptan-7-yl)methyl)acetamide
3-((2-amino-3-chloropyridin-4-yl)thio)-6-((1S,6R,7R)-7-(aminomethyl)-7-(2-fluorophenyl)-3-azabicyclo[4.1.0]heptan-3-yl)pyrazin-2-amine
4-((6-((1S,6R,7R)-7-(aminomethyl)-7-(2-fluorophenyl)-3-azabicyclo[4.1.0]heptan-3-yl)pyrido[2,3-b]pyrazin-2-yl)thio)-3-chloropyridin-2-amine
((S)-1-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-yl)pyrrolidin-3-yl)methanol
4-((6-((1R,6S,7S)-7-(aminomethyl)-7-(2-fluorophenyl)-3-azabicyclo[4.1.0]heptan-3-yl)-1,5-naphthyridin-2-yl)thio)-3-chloropyridin-2-amine
((1S,6R,7R)-3-(5-((3-chloro-2-(1H-pyrazol-1-yl)-1,4-dihydropyridin-4-yl)thio)pyrazin-2-yl)-7-(2-fluorophenyl)-3-azabicyclo[4.1.0]heptan-7-yl)methanamine
((1S,6R,7R)-3-(3-(2-(difluoromethyl)-4-fluoro-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(1-phenyl-1H-pyrazol-4-yl)-1H-pyrazolo[3,4-b]pyrazin-6-yl)-3-azabicyclo[4.1.0]heptan-7-yl)methanamine
((1S,6R,7R)-3-(3-(4-chloro-1,4-dihydrobenzo[c][1,2,5]thiadiazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-7-chlorobenzo[d]thiazol-2(3H)-one
((1S,6R,7R)-3-(3-(4-chloro-2-(2-methoxyethyl)-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(6-(2-methyl-2H-indazol-5-yl)-1,5-naphthyridin-2-yl)-3-azabicyclo[4.1.0]heptan-7-yl)methanamine
4-((2-((1S,6R,7R)-7-(aminomethyl)-7-(2-fluorophenyl)-3-azabicyclo[4.1.0]heptan-3-yl)pyrido[2,3-b]pyrazin-6-yl)thio)-3-chloropyridin-2-amine
((1S,6R,7R)-3-(3-(7-chloro-1-methyl-1H-benzo[d]imidazol-6-yl)-1H-pyrazolo[3,4-b]pyrazin-6-yl)-7-(2,5-difluorophenyl)-3-azabicyclo[4.1.0]heptan-7-yl)methanamine
4-((6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)thio)-3-chloropyridin-2-amine
((1S,6R,7R)-3-(6-((3-chloro-2-(1H-pyrazol-1-yl)pyridin-4-yl)thio)pyrido[2,3-b]pyrazin-2-yl)-7-(2-fluorophenyl)-3-azabicyclo[4.1.0]heptan-7-yl)methanamine
((1S,6R,7R)-3-(3-(3-fluoro-4-methoxyphenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-((4-chloro-2-methyl-2H-indazol-5-yl)thio)pyrido[2,3-b]pyrazin-2-yl)-7-(2-fluorophenyl)-3-azabicyclo[4.1.0]heptan-7-yl)methanamine
4-((5-((1S,6R,7R)-7-(aminomethyl)-7-(2-fluorophenyl)-3-azabicyclo[4.1.0]heptan-3-yl)pyrazin-2-yl)thio)-3-chloropyridin-2-amine
((1S,6R,7R)-3-(5-((3-chloro-2-(1H-pyrazol-1-yl)pyridin-4-yl)thio)pyrazin-2-yl)-7-(2,5-difluorophenyl)-3-azabicyclo[4.1.0]heptan-7-yl)methanamine
((1R,7S)-4-(5-((3-chloro-2-(1H-pyrazol-1-yl)pyridin-4-yl)thio)pyrazin-2-yl)-8-(2-fluorophenyl)-4-azabicyclo[5.1.0]octan-8-yl)methanamine
((1R,7S)-4-(3-(4-chloro-2-methyl-2H-indazol-5-yl)-1H-pyrazolo[3,4-b]pyrazin-6-yl)-8-(2-fluorophenyl)-4-azabicyclo[5.1.0]octan-8-yl)methanamine
((1S,6R,7R)-3-(3-(4-chloro-1H-indol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-(3,4-dihydro-1,5-naphthyridin-1(2H)-yl)pyrido[2,3-b]pyrazin-2-yl)-7-(2-fluorophenyl)-3-azabicyclo[4.1.0]heptan-7-yl)methanamine
((1S,6R,7R)-7-(2-fluorophenyl)-3-(6-(pyridin-4-ylthio)-3,4-dihydropyrido[2,3-b]pyrazin-2-yl)-3-azabicyclo[4.1.0]heptan-7-yl)methanamine
2-((1S,6R,7R)-7-(aminomethyl)-7-(2-fluorophenyl)-3-azabicyclo[4.1.0]heptan-3-yl)-7-(2,3-dichlorophenyl)-4H-pyrido[1,2-a]pyrimidin-4-one
((1S,6R,7R)-3-(6-(4-fluorophenoxy)pyrido[2,3-b]pyrazin-2-yl)-7-(2-fluorophenyl)-3-azabicyclo[4.1.0]heptan-7-yl)methanamine
7-((2-amino-3-chloropyridin-4-yl)thio)-2-((1S,6R,7R)-7-(aminomethyl)-7-(2-fluorophenyl)-3-azabicyclo[4.1.0]heptan-3-yl)-4H-pyrido[1,2-a]pyrimidin-4-one
6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one
6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one
((1S,6R,7R)-3-(3-(4-chloro-1-cyclopropyl-1H-indazol-5-yl)-1H-pyrazolo[3,4-b]pyrazin-6-yl)-7-(2-fluorophenyl)-3-azabicyclo[4.1.0]heptan-7-yl)methanamine
3-((2-amino-3-chloropyridin-4-yl)thio)-6-((1S,6R,7R)-7-(aminomethyl)-7-(2,5-difluorophenyl)-3-azabicyclo[4.1.0]heptan-3-yl)pyrazin-2-amine
((1S,6R,7R)-7-(2-fluorophenyl)-3-(6-(2-methyl-2H-indazol-5-yl)pyrido[2,3-b]pyrazin-2-yl)-3-azabicyclo[4.1.0]heptan-7-yl)methanamine
((1S,6R,7R)-3-(3-(2,3-dichlorophenyl)imidazo[1,5-a]pyrazin-8-yl)-7-(2-fluorophenyl)-3-azabicyclo[4.1.0]heptan-7-yl)methanamine
((1S,6R,7R)-3-(3-(1-(difluoromethyl)-1H-benzo[d]imidazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1-(difluoromethyl)-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-((3-fluoro-2-methylpyridin-4-yl)thio)pyrido[2,3-b]pyrazin-2-yl)-7-(2-fluorophenyl)-3-azabicyclo[4.1.0]heptan-7-yl)methanamine
((1S,6R,7R)-3-(3-(4-chloro-2-cyclopropyl-2H-indazol-5-yl)-1H-pyrazolo[3,4-b]pyrazin-6-yl)-7-(2-fluorophenyl)-3-azabicyclo[4.1.0]heptan-7-yl)methanamine
4-((5-((1S,6R,7R)-7-(aminomethyl)-7-(2-fluorophenyl)-3-azabicyclo[4.1.0]heptan-3-yl)pyrazin-2-yl)thio)-3-chlorobenzamide
((1S,6R,7R)-7-(2-fluorophenyl)-3-(5-(quinolin-5-ylthio)pyrazin-2-yl)-3-azabicyclo[4.1.0]heptan-7-yl)methanamine
((1S,6R,7R)-3-(5-((3-fluoro-2-methylpyridin-4-yl)thio)pyrazin-2-yl)-7-(2-fluorophenyl)-3-azabicyclo[4.1.0]heptan-7-yl)methanamine
((1S,6R,7R)-7-(2,5-difluorophenyl)-3-(5-((3-fluoro-2-methylpyridin-4-yl)thio)pyrazin-2-yl)-3-azabicyclo[4.1.0]heptan-7-yl)methanamine
((1S,6R,7R)-3-(6-(2,4-dimethyl-2H-indazol-5-yl)pyrido[2,3-b]pyrazin-2-yl)-7-(2-fluorophenyl)-3-azabicyclo[4.1.0]heptan-7-yl)methanamine
((1S,6R,7R)-7-(2,5-difluorophenyl)-3-(6-((3-fluoro-2-methylpyridin-4-yl)thio)pyrido[2,3-b]pyrazin-2-yl)-3-azabicyclo[4.1.0]heptan-7-yl)methanamine
((1S,6R,7R)-3-(5-((4-chloro-2-methyl-2H-indazol-5-yl)thio)pyrazin-2-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2H-indazol-2-yl)-N,N-dimethylacetamide
3-((2-amino-3-chloropyridin-4-yl)thio)-6-((1S,6R,7R)-7-(aminomethyl)-7-(2,3-difluorophenyl)-3-azabicyclo[4.1.0]heptan-3-yl)pyrazin-2-amine
6-((1S,6R,7R)-7-(aminomethyl)-7-(2,5-difluorophenyl)-3-azabicyclo[4.1.0]heptan-3-yl)-3-((3-chloro-2-cyclopropylpyridin-4-yl)thio)pyrazin-2-amine
6-((1S,6R,7R)-7-(aminomethyl)-7-(2,5-difluorophenyl)-3-azabicyclo[4.1.0]heptan-3-yl)-3-((3-fluoro-2-methylpyridin-4-yl)thio)pyrazin-2-amine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1H-indazol-1-yl)-N,N-dimethylacetamide
6-((1R,5S,6r)-6-(aminomethyl)-6-(4-methylthiazol-2-yl)-3-azabicyclo[3.1.0]hexan-3-yl)-3-(2,3-dichlorophenyl)-2-methylpyrimidin-4(3H)-one
3-((2-amino-3-chloropyridin-4-yl)thio)-6-((1R,5S,6r)-6-(aminomethyl)-6-(4-methylthiazol-2-yl)-3-azabicyclo[3.1.0]hexan-3-yl)pyrazin-2-amine
((1S,6R,7R)-7-(2-fluorophenyl)-3-(6-(8-methoxyquinolin-6-yl)pyrido[2,3-b]pyrazin-2-yl)-3-azabicyclo[4.1.0]heptan-7-yl)methanamine
((1S,6R,7R)-7-(2-fluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine
((1R,6S,7R)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7R)-3-(3-(4-chloropyrazolo[1,5-a]pyridin-5-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(3-(4-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(4-chloro-2-methylpyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
6-(6-((1R,5S,6r)-6-(aminomethyl)-6-(4-methylthiazol-2-yl)-3-azabicyclo[3.1.0]hexan-3-yl)-4,7-dihydro-1H-pyrazolo[3,4-b]pyrazin-3-yl)-5-fluoroisoquinolin-1(2H)-one
(3-(3-(8-chloroquinolin-5-yl)-1H-pyrazolo[3,4-b]pyrazin-6-yl)-7-(4-methylthiazol-2-yl)-3-azabicyclo[4.1.0]heptan-7-yl)methanamine
4-(6-(7-(aminomethyl)-7-(4-methyl-2,3-dihydrothiazol-2-yl)-3-azabicyclo[4.1.0]heptan-3-yl)-4,7-dihydro-1H-pyrazolo[3,4-b]pyrazin-3-yl)naphthalene-1-sulfonyl fluoride
((1S,6R,7S)-3-(3-(7-chloro-2-methylbenzo[d]thiazol-6-yl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(3,4-dichloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine
(7-(4-chlorothiazol-2-yl)-3-(3-(8-fluoroquinolin-5-yl)-4H-114-pyrazolo[4,5-b]pyrazin-6-yl)-3-azabicyclo[4.1.0]heptan-7-yl)methanamine
7-((5-((1R,5S,6r)-6-(aminomethyl)-6-(4-methylthiazol-2-yl)-3-azabicyclo[3.1.0]hexan-3-yl)pyrazin-2-yl)thio)-8-chloro-N,N-dimethylimidazo[1,2-a]pyridine-2-carboxamide
5-(6-((1S,6R,7S)-7-(aminomethyl)-7-(4-methylthiazol-2-yl)-3-azabicyclo[4.1.0]heptan-3-yl)-2,4-dihydro-1H-pyrazolo[3,4-b]pyrazin-3-yl)quinolin-8-yl sulfurofluoridate
5-(6-((1S,6R,7R)-7-(aminomethyl)-7-(2-fluorophenyl)-3-azabicyclo[4.1.0]heptan-3-yl)-1,4-dihydro-712-pyrazolo[3,4-b]pyrazin-3-yl)quinolin-8-yl sulfurofluoridate
4-(6-(7-(aminomethyl)-7-(4-methyl-2H-114-thiazol-2-yl)-3-azabicyclo[4.1.0]heptan-3-yl)-1H-pyrazolo[3,4-b]pyrazin-3-yl)-5,6-dichlorocyclohexa-1,4-dien-1-yl sulfurofluoridate
((1S,6R,7R)-3-(3-(3-chloro-2-(1H-pyrazol-1-yl)pyridin-4-yl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(2,3-dichlorophenyl)-1H-pyrazolo[3,4-b]pyrazin-6-yl)-6-phenyl-3-azabicyclo[3.1.0]hexan-6-yl)methanamine
((1R,5S,6s)-3-(3-(4-chloro-2-methyl-2H-indazol-5-yl)-1H-pyrazolo[3,4-b]pyrazin-6-yl)-6-phenyl-3-azabicyclo[3.1.0]hexan-6-yl)methanamine
((1S,6R,7R)-3-(3-(2-chloro-4-(2H-114-pyrrol-1-yl)phenyl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(8-fluoroquinolin-6-yl)-1,7-dihydro-412-pyrazolo[4,3-b]pyrazin-6-yl)-6-phenyl-3-azabicyclo[3.1.0]hexan-6-yl)methanamine
((1R,5S,6s)-3-(3-(5-chloroquinoxalin-6-yl)-1H-pyrazolo[3,4-b]pyrazin-6-yl)-6-phenyl-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloropyrazolo[1,5-a]pyridin-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(8-fluorooctahydro-2H-112-quinolin-4-yl)-6,7-dihydro-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(5-chloroquinoxalin-6-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(7-chloro-2-methylbenzo[d]thiazol-6-yl)-1,7-dihydro-412-pyrazolo[4,3-b]pyrazin-6-yl)-6-(4-methylthiazol-2-yl)-3-azabicyclo[3.1.0]hexan-6-yl)methanamine
6-(6-((1R,5S,6r)-6-(aminomethyl)-6-((S)-4-methyl-2,3-dihydrothiazol-2-yl)-3-azabicyclo[3.1.0]hexan-3-yl)-3,7-dihydro-114,212-pyrazolo[3,4-b]pyrazin-3-yl)isoquinolin-1(2H)-one
((1R,5S,6r)-3-(3-(4-chlorobenzo[c]isothiazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(7-chloro-2-(difluoromethyl)-4H-214-indazol-6-yl)-2,3-dihydro-1H-pyrazolo[3,4-b]pyrazin-6-yl)-6-((R)-4-methyl-312-thiazol-2-yl)-3-azabicyclo[3.1.0]hexan-6-yl)methanamine
((1R,5S,6r)-3-(3-(2-(difluoromethyl)-4-fluoro-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-2-(difluoromethyl)-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chlorobenzo[c][1,2,5]thiadiazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-2-(difluoromethyl)-4,7-dihydro-2H-indazol-5-yl)-1H-pyrazolo[3,4-b]pyrazin-6-yl)-6-(isothiazol-3-yl)-3-azabicyclo[3.1.0]hexan-6-yl)methanamine
6-((1R,5S,6r)-6-(aminomethyl)-6-(4-methylthiazol-2-yl)-3-azabicyclo[3.1.0]hexan-3-yl)-3-(7-chloro-2-methylbenzo[d]thiazol-6-yl)-5-methyl-1,5-dihydro-4H-pyrazolo[3,4-d]pyrimidin-4-one
6-((1R,5S,6r)-6-(aminomethyl)-6-(4-chlorothiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
6-((1R,5S,6r)-6-(aminomethyl)-6-(4-(trifluoromethyl)thiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
6-((1R,5S,6r)-6-(aminomethyl)-6-(4-methylthiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
2-((1R,5S,6r)-6-(aminomethyl)-6-(4-methylthiazol-2-yl)-3-azabicyclo[3.1.0]hexan-3-yl)-5-(4-chloro-2-methyl-2H-indazol-5-yl)-3-methyl-3,7-dihydro-4H-pyrrolo[2,3-d]pyrimidin-4-one
((1R,5S,6r)-3-(3-(4-chloro-2-methyl-2H-indazol-5-yl)-1H-pyrazolo[3,4-b]pyrazin-6-yl)-6-(1-methyl-1H-pyrazol-3-yl)-3-azabicyclo[3.1.0]hexan-6-yl)methanamine
((1R,5S,6r)-3-(3-(7-chloro-2-methylbenzo[d]thiazol-6-yl)-1H-pyrazolo[3,4-b]pyrazin-6-yl)-6-(1-methyl-1H-pyrazol-3-yl)-3-azabicyclo[3.1.0]hexan-6-yl)methanamine
((1R,5S,6r)-3-(3-(3-chloro-2-methoxypyridin-4-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(3-fluoro-2-methyl-1,2-dihydropyridin-4-yl)-1H-pyrazolo[3,4-b]pyrazin-6-yl)-6-(4-methylthiazol-2-yl)-3-azabicyclo[3.1.0]hexan-6-yl)methanamine
2-(5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-4-chloro-2H-indazol-2-yl)acetonitrile
((1R,5S,6r)-3-(3-(7-chlorobenzo[d][1,2,3]thiadiazol-6-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-1-methyl-1H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7S)-3-(3-(6-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
((1R,5S,6r)-6-(4-methylthiazol-2-yl)-3-(3-(pyrazolo[1,5-a]pyridin-4-yl)-1H-pyrazolo[3,4-b]pyrazin-6-yl)-3-azabicyclo[3.1.0]hexan-6-yl)methanamine
((1R,5S,6r)-3-(3-(7-chloro-1-methyl-1H-benzo[d]imidazol-6-yl)-1H-pyrazolo[3,4-b]pyrazin-6-yl)-6-(4-methylthiazol-2-yl)-3-azabicyclo[3.1.0]hexan-6-yl)methanamine
1-((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)-N-methylmethanamine
((1R,5S,6r)-3-(3-(4-chloro-2-methyl-2H-indazol-5-yl)-1H-pyrazolo[3,4-b]pyrazin-6-yl)-6-(2-methylthiazol-4-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(thiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-chlorothiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-(trifluoromethyl)thiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(5-fluorothiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(1,2,5-thiadiazol-3-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(pyridin-2-yl)-3-azabicyclo[3.1.0]hexan-6-yl)methanamine
((1R,5S,6r)-3-(3-(7-chloro-2-methylbenzo[d]thiazol-6-yl)-1H-pyrazolo[3,4-b]pyrazin-6-yl)-6-(pyridin-2-yl)-3-azabicyclo[3.1.0]hexan-6-yl)methanamine
((1R,5S,6r)-3-(3-(4-chloro-2-methyl-3H-214-indazol-5-yl)-1H-pyrazolo[3,4-b]pyrazin-6-yl)-6-(5-methyl-1,4-dihydropyridin-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(6-chloropyridin-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(5-fluoropyridin-2-yl)-3-azabicyclo[3.1.0]hexan-6-yl)methanamine
((1R,5S,6r)-3-(3-(4-chloro-2-methyl-3,7-dihydro-2H-indazol-5-yl)-1H-pyrazolo[3,4-b]pyrazin-6-yl)-6-(6-methyl-1,6-dihydropyridin-2-yl)-3-azabicyclo[3.1.0]hexan-6-yl)methanamine
4-((2-((1R,5S,6r)-6-(aminomethyl)-6-(4-methylthiazol-2-yl)-3-azabicyclo[3.1.0]hexan-3-yl)pyrido[2,3-b]pyrazin-6-yl)thio)-3-chloropyridin-2-amine
4-((5-((1R,5S,6r)-6-(aminomethyl)-6-(4-methylthiazol-2-yl)-3-azabicyclo[3.1.0]hexan-3-yl)-1,4-dihydropyrazin-2-yl)thio)-3-chloropyridin-2-amine
((1R,5S,6s)-3-(3-(4-chloro-2-methyl-2H-indazol-5-yl)-1H-pyrazolo[3,4-b]pyrazin-6-yl)-6-(2-fluorophenyl)-3-azabicyclo[3.1.0]hexan-6-yl)methanamine
((1S,6R,7R)-3-(3-(3-chloro-2-methoxypyridin-4-yl)-1H-pyrazolo[3,4-b]pyrazin-6-yl)-7-(2-fluorophenyl)-3-azabicyclo[4.1.0]heptan-7-yl)methanamine
((1R,5S,6r)-3-(3-(4-chloro-2-methyl-2H-indazol-5-yl)-1H-pyrazolo[3,4-b]pyrazin-6-yl)-6-(5-methyl-1,3,4-thiadiazol-2-yl)-3-azabicyclo[3.1.0]hexan-6-yl)methanamine
((1S,6R,7S)-3-(7-(8-fluoroquinolin-5-yl)-5H-pyrrolo[2,3-b]pyrazin-3-yl)-7-((R)-4-methyl-2,3-dihydrothiazol-2-yl)-3-azabicyclo[4.1.0]heptan-7-yl)methanamine
5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-1,3-dimethyl-1,3,4,7-tetrahydro-2H-benzo[d]imidazol-2-one
5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)quinoline-8-carboxamide
((1R,5S,6r)-3-(3-(4-chloro-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-1-(difluoromethyl)-1H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7R)-7-(2-fluorophenyl)-3-(7-(8-fluoroquinolin-5-yl)-5H-pyrrolo[2,3-b]pyrazin-3-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-4,7-dihydro-1H-pyrazolo[3,4-b]pyrazin-3-yl)quinoline-8-sulfonyl fluoride
((1S,6R,7R)-3-(3-(3-chloro-2-fluoropyridin-4-yl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(8-fluoroquinolin-5-yl)-1H-pyrazolo[3,4-b]pyrazin-6-yl)-6-phenyl-3-azabicyclo[3.1.0]hexan-6-yl)methanamine
((1R,5S,6r)-3-(3-(5-chloroquinoxalin-6-yl)-1H-pyrazolo[3,4-b]pyrazin-6-yl)-6-(1-methyl-1H-pyrazol-3-yl)-3-azabicyclo[3.1.0]hexan-6-yl)methanamine
4-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-3-chlorobenzamide
5-(6-((1R,5S,6r)-6-(aminomethyl)-6-((R)-4-methyl-2,3-dihydrothiazol-2-yl)-3-azabicyclo[3.1.0]hexan-3-yl)-1H-pyrazolo[3,4-b]pyrazin-3-yl)-1-methylindolin-2-one
((1R,5S,6r)-3-(3-(4-chloro-2-methyl-3H-214-indazol-5-yl)-1H-pyrazolo[3,4-b]pyrazin-6-yl)-6-(2-methyloxazol-4-yl)-3-azabicyclo[3.1.0]hexan-6-yl)methanamine
((1R,5S,6r)-3-(6-((3-chloro-2-(1H-pyrazol-1-yl)pyridin-4-yl)thio)pyrido[2,3-b]pyrazin-2-yl)-6-(4-methylthiazol-2-yl)-3-azabicyclo[3.1.0]hexan-6-yl)methanamine
((1R,5S,6r)-3-(3-(3-chloro-2-(1H-pyrazol-1-yl)pyridin-4-yl)-1H-pyrazolo[3,4-b]pyrazin-6-yl)-6-(4-methylthiazol-2-yl)-3-azabicyclo[3.1.0]hexan-6-yl)methanamine
4-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-3-chloropyridin-2-amine
((1R,5S,6r)-3-(3-(4-chloro-2-(2,2,2-trifluoroethyl)-2H-indazol-5-yl)-1,7-dihydro-412-pyrazolo[4,3-b]pyrazin-6-yl)-6-(4-methylthiazol-2-yl)-3-azabicyclo[3.1.0]hexan-6-yl)methanamine
((1R,5S,6r)-3-(3-(3-chloropyridin-4-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(5-((3-chloro-2-(1H-pyrazol-1-yl)-1,4-dihydropyridin-4-yl)thio)-1,2-dihydropyrazin-2-yl)-6-(4-methylthiazol-2-yl)-3-azabicyclo[3.1.0]hexan-6-yl)methanamine
((1S,6R,7R)-7-(2-fluorophenyl)-3-(3-(8-(methylsulfonyl)quinolin-5-yl)-1H-pyrazolo[3,4-b]pyrazin-6-yl)-3-azabicyclo[4.1.0]heptan-7-yl)methanamine
((1S,6R,7R)-3-(3-(5-chloro-6-(1H-pyrrol-1-yl)-1,2-dihydropyridin-4-yl)-1H-pyrazolo[3,4-b]pyrazin-6-yl)-7-(2-fluorophenyl)-3-azabicyclo[4.1.0]heptan-7-yl)methanamine
6-((1S,6R,7R)-7-(aminomethyl)-7-(2-fluorophenyl)-3-azabicyclo[4.1.0]heptan-3-yl)-3-((5-chloroquinoxalin-6-yl)thio)pyrazin-2-amine
6-((1S,6R,7R)-7-(aminomethyl)-7-(2-fluorophenyl)-3-azabicyclo[4.1.0]heptan-3-yl)-3-(quinolin-4-ylthio)pyrazin-2-amine
5-((3-amino-5-((1S,6R,7R)-7-(aminomethyl)-7-(2-fluorophenyl)-3-azabicyclo[4.1.0]heptan-3-yl)pyrazin-2-yl)thio)quinoline-8-carboxamide
6-((1R,5S,6r)-6-(aminomethyl)-6-(4-methylthiazol-2-yl)-3-azabicyclo[3.1.0]hexan-3-yl)-5-chloro-3-(2,3-dichlorophenyl)-2-methylpyrimidin-4(3H)-one
((1S,6R,7R)-7-(2,5-difluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine
((1R,5S,6R)-3-(3-(4-chloro-2-cyclopropyl-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7R)-3-(6-(3-fluoro-2-methylpyridin-4-yl)pyrido[2,3-b]pyrazin-2-yl)-7-(2-fluorophenyl)-3-azabicyclo[4.1.0]heptan-7-yl)methanamine
   ((1S,6R,7R)-3-(6-(2-(1H-pyrazol-1-yl)pyridin-4-yl)pyrido[2,3-b]pyrazin-2-yl)-7-(2-fluorophenyl)-3-azabicyclo[4.1.0]heptan-7-yl)methanamine
   5-(6-((1S,6R,7R)-7-(aminomethyl)-7-(2,5-difluorophenyl)-3-azabicyclo[4.1.0]heptan-3-yl)-1H-pyrazolo[3,4-b]pyrazin-3-yl)-3,4-dihydroquinolin-2(1H)-one
      or a pharmaceutically acceptable salt, tautomer, solvate, or stereoisomer thereof.

Compounds of the invention may be used in the form of prodrugs. A prodrug may be a pharmacologically inactive derivative of a biologically active substance (the “parent drug” or “parent molecule”, i.e. the compound of the invention) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The invention also includes all suitable isotopic variations of a compound of the invention. Examples of isotopes that can be incorporated into compounds of the disclosure include isotopes such as ²H, ³H, ¹³C, ¹⁴C, ⁵N, ¹⁷O, ¹⁸O, ³¹P, ³²P, ³⁵S, ¹⁸F and ³⁶Cl, respectively. Certain isotopic variations of the disclosure, for example, those in which a radioactive isotope such as ³H or ¹⁴C is incorporated, are useful in drug and/or substrate tissue distribution studies. Further, substitution with isotopes such as deuterium ²H, may afford certain therapeutic advantages resulting from greater metabolic stability. Isotopic variations of the compounds of the invention can generally be prepared by conventional procedures such as by the illustrative methods and the preparations described in the Descriptions and in the Examples hereafter using appropriate isotopic variations of suitable reagents.

The present invention includes within its scope solvates of the compounds of Formula (I) or of the relative salts, for example, hydrates, alcoholates and the like.

In addition, the compounds disclosed herein may exist as tautomers and all tautomeric forms are intended to be encompassed by the scope of the invention, even though only one tautomeric structure is depicted. For example, a reference to 2-hydroxypyridine also includes pyridin-2-one as its tautomeric form and a reference to 4-hydroxypyridine also includes pyridin-4-one as its tautomeric form. Similarly, a reference to a hydroxypyrimidine derivative also includes the corresponding pyrimidinone tautomer, a reference to 2,4-dihydro-3H-1,2,4-triazol-3-one also includes the corresponding 1H-1,2,4-triazol-5-ol, and so on. A particular embodiment of Formula (I) encompasses compounds of structures indicated below:

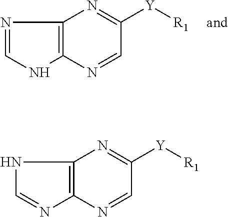

wherein R₁ and Y are as defined hereinabove.

The compounds disclosed herein may exist in different isomeric forms, all of which are encompassed by the present invention. In particular, any reference to the compound of the present invention is intended to include all its possible resonance forms.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers with all possible isomers and mixtures thereof, including optical isomers and atropoisomers, all such stereoisomers being included in the present invention.

Atropoisomers are stereoisomers arising because of hindered rotation about a single bond: compounds 411, 412, 433a and 433b are specific examples of atropoisomers according to the invention.

Pure stereoisomeric forms of the compounds and intermediates of this invention may be obtained by the application of art-known procedures and are intended to be encompassed by the scope of the invention. In particular, “pure stereoisomeric form” or “stereoisomerically pure” indicate a compound having stereoisomeric excess of at least 80%, preferably of at least 85%. For instance, enantiomers may be separated from each other by the selective crystallization of their diastereomeric salts or by chromatographic techniques using chiral stationary phases. Pure stereochemically isomeric forms may also be derived from the corresponding pure stereochemically isomeric forms of the appropriate starting materials, provided that the reaction occurs stereospecifically. The term “enantiomerically pure” shall be interpreted in a similar way, having regard to the enantiomeric ratio.

When any variable (e.g. R₁ and R₂, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” should be taken to be equivalent to the phrase “unsubstituted or substituted with one or more substituents” and in such cases the preferred embodiment will have from/zero to three substituents. More particularly, there are zero to two substituents.

According to the invention, “ [symbol] ” represent a single or a double bond.

The expression “one or more substituents” refers to in particular to 1, 2, 3, 4 or more substituents, in particular to 1, 2, 3 or 4 substituents, more in particular 1, 2 or 3 substituents.

As used herein “X₁ is a single bond” indicates that, in the general Formula (I), Cy is directly linked via a single bond to R₁; if “L is a single bond”, the carbon atoms substituted, respectively, with R₃ and R₄ are linked together forming a fused cyclopropyl ring, as in the structure below:

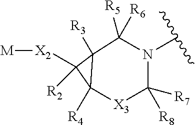

As used herein, if two residues taken together represent a single bond, this means that the two atoms to which they are attached are connected by a single bond or by an additional bond thus forming a multiple bond.

As used herein, “alkyl” is intended to include both branched and straight-chain saturated aliphatic hydrocarbon groups having the specified number of carbon atoms. For example, “C₁₋₆alkyl” is defined to include groups having 1, 2, 3, 4, 5 or 6 carbons in a linear or branched arrangement and specifically includes methyl, ethyl, n-propyl, i-propyl, n-butyl, t-butyl, i-butyl, pentyl, hexyl, and so on. Preferably, “C₁₋₆alkyl” refers to “C₁₋₄alkyl” or “C₁₋₃alkyl”. “C₁₋₄alkyl” is defined to include groups having 1, 2, 3 or 4 carbons in a linear or branched arrangement. For example, “C₁₋₄ alkyl” specifically includes methyl, ethyl, n-propyl, i-propyl, n-butyl, t-butyl, i-butyl, and so on. “C₁₋₃alkyl” is defined to include groups having 1, 2, or 3 carbons in a linear or branched arrangement. For example, “C₁₋₃ alkyl” specifically includes methyl, ethyl, n-propyl, i-propyl, and so on. Preferred alkyl groups are methyl, ethyl, i-propyl, t-butyl or i-butyl.

As used herein, “alkoxy” represents an alkyl group of indicated number of carbon atoms attached through an oxygen bridge. “Alkoxy” therefore encompasses the definitions of alkyl above. C₁₋₆ alkoxy group is preferably a linear or branched C₁₋₄alkoxy group, more preferably a C₁₋₃alkoxy group, still more preferably a C₁₋₂ alkoxy group. Examples of suitable alkoxy groups include, but are not limited to methoxy, ethoxy, n-propoxy, i-propoxy, n-butoxy, s-butoxy or t-butoxy. Preferred alkoxy groups include methoxy, ethoxy and t-butoxy.

As used herein, the terms “haloC₁₋₆alkyl”, “haloC₁₋₆alkoxy” and variants thereof such as “C₁₋₆haloalkyl” mean a C₁₋₆alkyl or C₁₋₆alkoxy group in which one or more (in particular, 1 to 3) hydrogen atoms have been replaced by halogen atoms, especially fluorine or chlorine atoms. HaloC₁₋₆alkoxy group is preferably a linear or branched haloC₁₋₄alkoxy group, more preferably a haloC₁₋₃alkoxy group, still more preferably a haloC₁₋₂alkoxy group, for example OCF₃, OCHF₂, OCH₂F, OCH₂CH₂F, OCH₂CHF₂ or OCH₂CF₃, and most especially OCF₃ or OCHF₂. HaloC₁₋₆alkyl group is preferably a linear or branched haloC₁₋₃alkyl group, more preferably a haloC₁₋₂alkyl group for example, CF₃, CHF₂, CH₂F, CH₂CH₂F, CH₂CHF₂, CH₂CF₃ or CH(CH₃)CF₃, and most especially CF₃, CHF₂ or CH(CH₃)CF₃.

As used herein, heteroaryl-C₁₋₆alkoxy is a C₁₋₆alkoxy wherein at leas one hydrogen is substituted with an heteroaryl group, preferably a substituted heteroaryl group.

As used herein, the term “hydroxyC₁₋₆alkyl” means a C₁₋₆alkyl group in which one or more (in particular, 1 to 3) hydrogen atoms have been replaced by hydroxy groups. Preferably, the hydroxyC₁₋₆alkyl is a hydroxyC₁₋₄alkyl, meaning a C₁₋₄alkyl group in which one or more (in particular, 1 to 2) hydrogen atoms have been replaced by hydroxy groups, even more preferably is hydroxy-ethyl. Illustrative examples include, but are not limited to CH₂OH, CH₂CH₂OH, CH(CH₃)OH, C(CH₃)₂OH and CHOHCH₂OH. In a further preferred embodiment the hydroxy is substituted with an alkyl group, i.e. is an alkoxy group, such as for example C₁₋₃alkyl-OC₁₋₃alkyl.

As used herein, the term “heteroaryl-C₁₋₆alkoxy” means a C₁₋₆alkoxy group in which one hydrogen atom is replaced by an heteroaryl group, wherein said aryl or heteroaryl can be further substituted by, for example, methyl, halogen, hydroxyl or amine.

As used herein, heteroaryloxy means heteroaryl-O—, wherein the heteroaryl can be optionally substituted with alkyl, halogen, amino, hydroxy and the like.

As used herein, heterocycloalkoxy means a heterocycloalkyl-O-group in which heterocycloalkyl is as defined herein.

As used herein, the terms “C₁₋₆alkylheteroaryl” or “C₁₋₆alkylaryl” mean a C₁₋₆alkyl group in which one hydrogen atom is replaced by an aryl or heteroaryl group, wherein said aryl or heteroaryl can be further substituted by, for example, methyl, halogen, hydroxyl or amine. Preferably the C₁₋₆alkylheteroaryl is a C₁₋₃alkylheteroaryl, such as a methylheteroaryl, ethylheteroaryl, propylheteroaryl or isopropylheteroaryl, wherein said aryl or heteroaryl can be further substituted by, for example, methyl, halogen, hydroxyl or amine. Preferably C₁₋₆alkylaryl is a C₁₋₃alkylaryl, even more preferably is a benzyl group.

As used herein, the term “aminoC₁₋₆alkyl” means a C₁₋₆alkyl group in which one or more (in particular, 1 to 3) hydrogen atoms have been replaced by small amino groups, such as NH₂, NHCH₃, N(CH₃)₂ and the like.

As used herein, the term “aryl” or “aromatic ring” means a monocyclic or polycyclic aromatic ring comprising carbon atoms and hydrogen atoms. If indicated, such aromatic ring may include one or more heteroatoms, then also referred to as “heteroaryl” or “heteroaromatic ring”, preferably, 1 to 3 heteroatoms, independently selected from nitrogen, oxygen, and sulfur, preferably nitrogen. As is well known to those skilled in the art, heteroaryl rings have less aromatic character than their all-carbon counter parts. Thus, for the purposes of the present invention, a heteroaryl group need only have some degree of aromatic character. Preferably, the ring component of aryl or heteroaryl groups comprises 5 or 6 members (i.e. atoms). Still preferably, aryl or heteroaryl groups are polycyclic aromatic rings. Illustrative examples of aryl groups are optionally substituted phenyls. Illustrative examples of heteroaryl groups according to the invention include optionally substituted thiophene, oxazole, thiazole, thiadiazole, imidazole, pyrazole, pyrimidine, pyrazine, pyridine and pyridine N-oxide. Thus, examples of monocyclic aryl optionally containing one or more heteroatoms, for example one or two heteroatoms, are a 5- or 6-membered aryl or heteroaryl group such as, but not limited to, phenyl, pyridyl, pyrimidinyl, pyrazinyl, pyridazinyl, pyrrolyl, thienyl, thiazolyl, thiadiazolyl, pyrazolyl, imidazolyl, triazolyl, tetrazolyl, furyl, isoxazolyl, oxadiazolyl and oxazolyl. Examples of polycyclic aromatic ring, optionally containing one or more heteroatoms, for example one or two heteroatoms, are a 8-10 membered aryl or heteroaryl group such as, but not limited to, benzimidazolyl, benzofurandionyl, benzofuranyl, benzofurazanyl, benzopyrazolyl, benzotriazolyl, benzothienyl, benzoxazolyl, benzoxazolonyl, benzothiazolyl, benzothiadiazolyl, benzoxadiazolyl, benzoisoxazolyl, benzoisothiazolyl, indolyl, indolinyl, indolizinyl, indazolyl, isobenzofuranyl, isoindolyl, isoindolinyl, isoquinolyl, quinazolinyl, quinolyl, quinoxalinyl, quinolizinyl, naphtyl, naphthyridinyl and phthalazinyl. Other examples of polycyclic heteroaromatic rings according to the invention are 2H-pyrazolo[3,4-b]pyridine, indazole, 2H-pyrazolo[3,4-c]pyridine, 6H-pyrrolo[3,4-b]pyridine, 6H-pyrrolo[3,4-b]pyrazine, 6H-pyrrolo[3,4-d]pyrimidine, 2H-pyrazolo[3,4-d]pyrimidine, 1,5-naphthyridine, imidazo[1,2-a]pyridine, furo[2,3-b]pyridine, 2,1,3-benzoxadiazole, benzo[d][1,2,3]triazole, [1,2,4]triazolo[4,3-a]pyridine, imidazo[1,2-a]pyridine, cinnoline, [1,2,4]triazolo[1,5-a]pyridine, benzimidazole. A preferred aryl according to the present invention is phenyl. A preferred heteroaryl according to the present invention is pyridyl.

The expressions “optionally substituted aryl”, “optionally substituted heteroaryl”, “optionally substituted aryloxy”, “optionally substituted heteroaryl-C₁₋₆alkyl”, “optionally substituted heteroaryl-C₁₋₆alkoxy” generically refer to aryl, heteroaryl or aryloxy groups wherein the aromatic or heteroaromatic ring may be substituted with one or more substituents. Examples of said substituents include alkyl, alkoxy, hydroxy, amino, trifluoromethyl, aryl, heteroaryl, hydroxyl, carboxyalkyl and the like.

As used herein, heterocycle, heterocyclic compound or ring structure, heterocycloalkyl and variants thereof refer to a saturated monocyclic or polycyclic compound that has atoms of at least two different elements as members of its ring(s).

Aryl or heteroaryl rings can also have a partially unsaturated structure and can thus be derived from the partially hydrogenated analogues of the before-listed aryl or heteroaryl groups but also from an aryl or heteroaryl ring fused with a cycloalkyl or heterocycloalkyl ring. Said rings might also contain a group selected from SO, SO₂ and C═O. Examples of said partially unsaturated aryl or heteroaryl derivatives include 2,3-dihydro-1H-indene, 2,3-dihydro-1H-inden-1-one, 2,3-dihydroisoindol-1-one, indoline, 1,2,3,4-tetrahydroquinoline, 1,2,3,4-tetrahydroisoquinoline, isoindoline, 1-methyl-indol-2-one, dihydroquinazoline, dihydroquinoxaline, 2,3-dihydrobenzofuran, benzo[d][1,3]dioxole, 1,3-dihydroisobenzofuran, 3,4-dihydro-2H-benzo[b][1,4]oxazine, 2,3,4,5-tetrahydrobenzo[f][1,4]oxazepine, quinazolin-4(3H)-one, 4,5,6,7-tetrahydro-1H-indazole, 4,5,6,7-tetrahydropyrazolo[1,5-a]pyrazine, 2,3,4,5-tetrahydro-1H-benzo[d]azepine, 6′,7′-dihydrospiro[azetidine-3,5′-pyrrolo[1,2-a]imidazole], 2,3-dihydrobenzo[b][1,4]dioxine, benzo[d]oxazol-2(3H)-one, 2H-benzo[b][1,4]oxazin-3(4H)-one, indolin-2-one, 1,2,3,4-tetrahydro-1,5-naphthyridine, 3′,4′-dihydro-2′H-spiro[azetidine-3,1′-pyrrolo[1,2-a]pyrazine], 3,4-dihydroquinolin-2(1H)-one, 4-methyl-2H-benzo[b][1,4]oxazin-3(4H)-one, quinoxalin-2(1H)-one, 4H-pyrido[1,2-a]pyrimidin-4-one, (6aS)-6a,7,8,9-tetrahydro-6H-pyrido[3,2-b]pyrrolo[1,2-d][1,4]oxazine, 3,4-dihydro-2H-1,5-naphthyridin-1-yl, dihydrofuro[2,3-b]pyridinyl, 1-methylpyridin-2(1H)-one, benzo[d]oxazol-2(3H)-one, pyridine-2(1H)one, 1,4-benzoxazin-3(4H)-one, isoquinolin-(2H)one, quinolin-(2H)one, 1,3-dihydro-2H-benzimidazol-2-one, benzoisothiazole-3(2H)-one-1,1-dioxide, benzo[d]thiazol-2(3H)-one phthalazine-1(2H)-one, pyrido[1,2-a]pyrimidin-4-one, and the like.

As used herein, the term “aryloxy” represents an aryl group as defined above attached through an oxygen bridge. Aryloxy therefore encompasses the definitions of aryl and heteroaryl above. Illustrative examples include phenoxy, napthyloxy, pyridinyloxy and so on.

It should be noted that different isomers of the various heterocycles may exist within the definitions as used throughout the specification. For example, pyrrolyl may be 1H-pyrrolyl or 2H-pyrrolyl.

It should also be noted that the radical positions on any molecular moiety used in the definitions may be anywhere on such moiety as long as it is chemically stable. For example, pyridyl includes 2-pyridyl, 3-pyridyl, 4-pyridyl.

As used herein, the term “halogen” refers to fluorine, chlorine, bromine and iodine, of which fluorine, chlorine and bromine are preferred.

The term “heteroatom” refers to an atom other than carbon or hydrogen in a ring structure or a saturated backbone as defined herein. Typical heteroatoms include N(H), O, S.

As used herein, “cycloalkoxy” represents a cycloalkyl group of the indicated number of carbons attached through an oxygen bridge. “Cycloalkoxy” therefore encompasses the definitions of cycloalkyl above and is preferably a C₁₋₆cycloalkoxy as cyclopropyloxy, cyclobutyloxy, cyclopentyloxy, cyclohexyloxy.

As used herein, the expression “C₁₋₆alkyl-NH₂”, “C₁₋₆alkyl-NH(CH₃)”, “C₁₋₆alkyl-N(CH₃)₂”, “C₁₋₆alkyl-NHCOCH₃” refers to a C₁₋₆alkyl as defined above wherein any one hydrogen is substituted respectively by a group of formula NH₂, NH(CH₃), N(CH₃)₂ or NHCOCH₃. Preferably, said “C₁₋₆alkyl-” is a “C₁₋₃alkyl-”, thus encompasses an alkyl of 1, 2 or 3 carbon atoms.

Included in the instant invention is the free base of compounds of Formula (I), and any of Formula (II)-(XXX) as well as the pharmaceutically acceptable salts and stereoisomers thereof. Some of the specific compounds exemplified herein are the protonated salts of amine compounds. Compounds containing one or more N atoms may be protonated on any one, some or all of the N atoms. The term “free base” refers to the amine compounds in non-salt form. The encompassed pharmaceutically acceptable salts not only include the salts exemplified for the specific compounds described herein, but also all the typical pharmaceutically acceptable salts of the free form of compounds of Formula (I), and any of Formula (II)-(XXX). The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base. In a preferred embodiment, the compounds of the invention have at least one acidic proton and the corresponding sodium or potassium salt can be formed, for example, by reaction with the appropriate base.

Thus, pharmaceutically acceptable salts of the compounds of this invention include the conventional non-toxic salts of the compounds of this invention as formed by reacting a basic instant compound with an inorganic or organic acid or an acid compound with an inorganic or organic base. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Convential non-toxic salts further include those derived from an inorganic base, such as potassium, sodium hydroxide, magnesium or calcium hydroxide, as well as salts prepared from organic bases, such as ethylene diamine, lysine, tromethamine, meglumine and the like. Preferably, a pharmaceutically acceptable salt of this invention contains one equivalent of a compound of Formula (I) or Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVIa), Formula (XVIb), Formula (XVII), Formula (XVIII), Formula (XIX), Formula (XX), Formula (XXI), Formula (XXII), Formula (XXIII), Formula (XXIV), Formula (XXV), Formula (XXVI), Formula (XXVII), Formula (XXVIII), Formula (XIX), Formula (XXX), Formula (XXXI) or Formula (XXXII) and 1, 2 or 3 equivalent of an inorganic or organic acid or base. More particularly, pharmaceutically acceptable salts of this invention are the tartrate, trifluoroacetate or the chloride salts.

When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

Preferably, compounds of the present invention, including salts, tautomers, stereoisomers and solvates thereof, are SHP2 inhibitors, meaning that for example they can inhibit the activity or function of SHP2. Then, the present invention relates to compounds for use as inhibitors of at least one SHP2 function and to a method of inhibiting at least one SHP2 function comprising the step of contacting SHP2 with a compound as described herein.

“SHP2” means “Src Homology-2-phosphatase” and is also known as SH-PTP2, SH-PTP3, Syp, PTPID, PTP2C, SAP-2 or PTPN11.

The functions of SHP2 are varied as SHP2 is involved in multiple signaling processes, such as RAS-ERK, JAK-STAT, PI3K-AKT, NF-κB, and mTOR pathways. SHP2 regulates cancer cell survival and proliferation primarily by activating the RAS-ERK signaling pathway (T. Matozaki, Y. Murata, Y. Saito, H. Okazawa, H. Ohnishi, Cancer Sci, 100 (2009), pp. 1786-1793). In the RAS-ERK pathway, SHP2 acts as a positive regulator at upstream to promote RAS-RAF-ERK kinase cascade signaling transduction. Therefore, SHP2 inhibition leads to dephosphorylation of ERK and suppression of the pro-oncogenic function of RAS-RAF-ERK pathway, resulting in cell growth inhibition and apoptosis induction in cancer cells. Recently, Chen et al. (Y. N. Chen, M. J. LaMarche, H. M. Chan, P. Fekkes, J. Garcia-Fortanet, M. G. Acker et al., Nature, 535 (2016), pp. 148-152) found that cancer cell lines sensitive to SHP2 depletion were also sensitive to EGFR depletion, which validated reports that RTK-driven cancer cells depend on SHP2 for survival. Furthermore, recent studies have shown that SHP2 is required for the growth of mutant KRAS-driven cancers while wild-type KRAS-amplified gastroesophageal cancer can be controlled through combined SHP2 and MEK inhibition (S. Mainardi, A. Mulero-Sanchez, A. Prahallad, G. Germano, A. Bosma, P. Krimpenfort, et al. Nat Med, 24 (2018), pp. 961-9; D. A. Ruess, G. J. Heynen, K. J. Ciecielski, J. Ai, A. Berninger, D. Kabacaoglu, et al. Nat Med, 24 (2018), pp. 954-960; G. S. Wong, J. Zhou, J. B. Liu, Z. Wu, X. Xu, T. Li, et al. Nat Med, 24 (2018), pp. 968-977). As a downstream target of several receptors, SHP2 is also involved in signaling in T-cells (M. Tajan, A. de Rocca Serra, P. Valet, T. Edouard, A. Yart, Eur J Med Genet, 58 (2015), pp. 509-525; R. J. Salmond, D. R. Alexander, Trends Immunol, 27 (2006), pp. 154-160). It is a downstream molecule in the PD-1 signaling pathway which not only suppresses T-cell activation but also causes T-cell anergy. SHP2-deficiency in T-cells triggered an anti-tumor immune response against colitis-associated cancer in mice (W. Liu, W. Guo, L. Shen, Z. Chen, Q. Luo, X. Luo, et al. Oncotarget, 8 (2017), pp. 7586-7597). Therefore, targeting SHP2 may restore or even enhance T-cell functions.

SHP2 inhibition may be assessed or measured by: the cell phenotype (as for example the phenotypes of proliferation and resistance to EGFR and c-MET co-inhibition, the mesenchymal phenotype in BTBC cells), cell proliferation, activity of SHP2, change in biochemical output produced by active SHP2, expression of SHP2, or binding of SHP2 with a natural binding partner may be monitored as a measure of SHP2 inhibition. In particular, inhibition of SHP2 activity or function can be measured by the IC₅₀ (concentration of inhibitor which reduces the activity of SHP2 to half-maximal level), as described in the assays hereinbelow or in the biochemical assays for SHP2 inhibition reported for example by Chen et al., Nature (535) 2016 or by Bagdanoff et al., J. Med. Chem. 2019, 62, 1781-1792. Preferably, compounds of the invention exhibit an IC₅₀ towards SHP2 lower than or equal to 10 μM. Preferred compounds exhibit an enzymatic IC₅₀ towards SHP2, as defined hereinbelow, lower than or equal to 3 μM (preferably lower than or equal to 0.5 μM or between 0.5 μM and 3 μM) and/or inhibition of SHP2 in cell based assays, as defined hereinbelow, with IC₅₀ lower than or equal to 5 μM (preferably lower than or equal to 1 μM or between 1 μM and 5 μM). Then, the compounds of the present invention, including salts, tautomers, stereoisomers and solvates thereof, may be for use in a method of inhibiting SHP2 activity. In other words, they may be for use in the prevention and/or treatment of any condition that would be ameliorated by SHP2 inhibition. In a preferred embodiment, the compound or a pharmaceutically acceptable salt, tautomer, solvate, or stereoisomer thereof as defined above is for use in inhibiting SHP2 activity. Inhibition of SHP2 activity may be measured with respect to a proper control, such as a subject affected by a disease or disorder mediated by the activity of SHP2 or a subject throughout the course of a therapy for a disease or disorder mediated by the activity of SHP2. Preferably, compounds of the invention inhibit SHP2 activity by at least 10%, 20%, 30%, 40%, 50%, 60%, 70%, 80%, 90% in respect to a proper control. More preferably, compounds of the invention inhibit SHP2 activity by approximately 10%, 20%, 30%, 40%, 50%, 60%, 70%, 80%, 90% in respect to a proper control.

Yet more preferably, compounds of the invention inhibit SHP2 activity by more than 90%, for instance by approximately 92%, 94%, 95%, 98%, 99% or 100% in respect to a proper control.

Therefore, the compounds of the invention can be used for the treatment of diseases and for carrying out biological assays, cellular assays, biochemical assays or the like.

It is an object of the invention a compound or a pharmaceutically acceptable salt, tautomer, solvate, or stereoisomer thereof as defined above for medical use.

Preferably, the compound or the pharmaceutically acceptable salt, tautomer, solvate, or stereoisomer thereof as defined above is for use in inhibiting SHP2 activity. Inhibition of SHP2 activity further leads to dephosphorylation of ERK and suppression of the pro-oncogenic function of RAS-RAF-ERK pathway. Then, inhibition of SHP2 activity may be measured by ERK dephosphorylation, wherein ERK phosphorylation may be evaluated by any method known in the art, for instance as described in the Examples below. Dephosphorylation of ERK may be measured with reference to any proper control.

More preferably, the compound or the pharmaceutically acceptable salt, tautomer, solvate, or stereoisomer thereof as defined above is for use in the treatment and/or prevention of a disease or disorder mediated by the activity of SHP2. Still preferably, the compound or the pharmaceutically acceptable salt, tautomer, solvate, or stereoisomer thereof as defined above is for use in the preparation of a medicament for treating or preventing a disease or disorder mediated by the activity of SHP2. Preferably, the disease or disorder mediated by the activity of SHP2 is selected from the group consisting of: cancer, cardiovascular disease, immunological disorder, fibrosis, an ocular disorder, systemic lupus erythematosus, diabetes, neutropenia, and combinations thereof. Yet preferably, the disease or disorder mediated by the activity of SHP2 is selected from the group consisting of: Noonan Syndrome, Leopard Syndrome, juvenile myelomonocytic leukemias, neuroblastoma, melanoma, head and neck squamous-cell carcinoma, acute myeloid leukemia, breast cancer, esophageal tumor, lung cancer, colon cancer, head cancer, gastric carcinoma, lymphoma, glioblastoma, gastric cancer, pancreatic cancer and combinations thereof. Preferably any one of said cancers is a primary cancer or a cancer metastasis.

A disease or disorder mediated by the activity of SHP2 indicates a condition in a subject in which modulation, in particular inhibition, of SHP2 activity can prevent, inhibit, ameliorate, slow down or eradicate the condition and/or the symptomology thereof. Treatment of said disease or disorder might comprise administering to the subject in need thereof a therapeutically effective amount of a compound of Formula (I) according to the invention.

In diseases or disorders mediated by the activity of SHP2, mutations are often observed at the N—SH2/PTP interface (e.g. E76D/E76K), resulting in constitutively active protein and abnormal proliferation. Cancers harboring “PTPN11 mutations” include but are not limited to: N58Y; D61Y, V; E69K; A72V, T, D; E76G, Q, K (ALL); G60A; D61Y; E69V; F71K; A72V; T73I; E76G, K; R289G; G503V (AML); G60R, D61Y, V, N; Y62D; E69K; A72T, V; T73I; E76K, V, G, A, Q; E139D; G503A, R; Q506P (JMML); G60V; D61V; E69K; F71L; A72V; E76A (MDS); Y63C (CMML); Y62C; E69K; T507K (neuroblastoma); V46L; N58S; E76V (Lung cancer); R138Q (melanoma); E76G (colon cancer). The compounds of the invention can exhibit affinity at low concentrations for wild type SHP2 and can also be active against mutant forms of the protein.

Another aspect of the present invention relates to a compound of the invention, including any pharmaceutically acceptable salt, tautomer, solvate or stereoisomer thereof, as defined hereinabove for use in a method of preventing/treating an SHP2-mediated disorder and/or disorders mediated by the pro-oncogenic function of RAS-RAF-ERK pathway.

Another aspect of the present invention relates to a method of preventing/treating an SHP2-mediated disorder comprising the step of administering to a patient in need thereof a therapeutically effective amount of a compound of the invention, including any pharmaceutically acceptable salt, tautomer, solvate or stereoisomer thereof, as defined hereinabove. In another aspect, the present invention relates to a method of preventing/treating an SHP2-mediated disorder comprising the step of administering to a patient in need thereof a therapeutically effective amount of a chemotherapeutic agent, as further defined below, in combination with a therapeutically effective amount of a compound of the invention.

Another aspect of the present invention relates to the use of compounds of the invention, including any pharmaceutically acceptable salts tautomer, solvate or stereoisomer thereof, as defined hereinabove in preventing/treating an SHP2-mediated disorder.

Another aspect of the present invention relates to the use of compounds of the invention, including any pharmaceutically acceptable salts tautomer, solvate or stereoisomer thereof, as defined hereinabove in the preparation of a medicament for preventing/treating an SHP2-mediated disorder.

In yet another aspect of the present invention, there are provided the compound, salt, solvate, stereoisomer or tautomer as defined above for use in the treatment and/or prevention of a disease or disorder selected from the group consisting of: cancer, cardiovascular disease, immunological disorder, fibrosis, an ocular disorder, systemic lupus erythematosus, diabetes, neutropenia and combinations thereof. Preferably, the disease or disorder is selected from the group consisting of: Noonan Syndrome, Leopard Syndrome, juvenile myelomonocytic leukemias, neuroblastoma, melanoma, head and neck squamous-cell carcinoma, acute myeloid leukemia, breast cancer, esophageal tumor, lung cancer, colon cancer, head cancer, gastric carcinoma, lymphoma, glioblastoma, gastric cancer, pancreatic cancer and combinations thereof.

Preferably, the cancer is a primary cancer or a cancer metastasis.

In certain embodiments the present invention relates to the aforementioned use/method, wherein said disorder is selected from Noonan Syndrome (NS) and Leopard Syndrome (LS).

In further embodiments, the present invention relates to the aforementioned use/method, wherein said SHP2-mediated disorders are those due to dysregulated cellular proliferation, including cancer. The cancer may be hormone-dependent or hormone-resistant, such as in the case of breast cancers. Preferably, the cancer is RTK-driven or KRAS-driven, such as KRAS amplified gastroesophageal cancer. In certain embodiments, the cancer is a solid tumor. In other embodiments, the cancer is a lymphoma or leukemia or a glioma. In certain embodiments, the cancer is a drug resistant phenotype of a cancer disclosed herein or known in the art. The cancer may be primary or metastatic. Tumor invasion, tumor growth, tumor metastasis, and angiogenesis may also be treated using the compositions and methods disclosed herein. Precancerous neoplasias may also be treated using the compositions and methods disclosed herein. Compounds of the invention can be used for the treatment of cancers selected from, but not limited to: Juvenile Myelomonocytic Leukemias (JMML); Acute Myeloid Leukemia (AML); Myelodysplastic Syndrome (MDS); B cell acute lymphoblastic leukemia (B-ALL); neuroblastoma; esophageal; breast cancer; lung cancer; colon cancer; gastric cancer, head and neck cancer; ovarian cancer; prostate cancer; cancers of the oral cavity and pharynx (lip, tongue, mouth, larynx, pharynx), stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); gastroesophageal cancer (preferably KRAS-amplified gastroesophageal cancer), cancers of the eye, brain, spinal cord, and other components of the central and peripheral nervous systems, as well as associated structures such as the meninges; thyroid and other endocrine glands, Hodgkin's disease, non-Hodgkin's lymphomas, multiple myeloma and hematopoietic malignancies including leukemias Chronic Lymphocytic Leukemia (CLL), Acute Lymphocytic Leukemia (ALL), Chronic Myelogenous Leukemia (CML), Acute Myelogenous Leukemia (AML), Chronic Myelomonocytic Leukemia (CMML), and lymphomas including lymphocytic, granulocytic and monocytic. Additional types of cancers which may be treated using the compounds and methods of the invention include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, Wilm's tumor, anaplastic large-cell lymphoma, colitis associated cancer. The compounds of the present invention may be useful in the treatment of any other disease or condition related to the aberrant activity of SHP2. Thus, as a further aspect, the invention relates to a method of treatment of a disorder selected from: NS; LS; JMML; AML; MDS; B-ALL; neuroblastoma; esophageal; breast cancer; lung cancer; colon cancer; gastric cancer; head and neck cancer.

A compound of the present invention, including any pharmaceutically acceptable salt, tautomer, solvate, or stereoisomer thereof, may be usefully combined with any another known therapy that is useful for the prevention/treatment of a disease or disorder mediated by the activity of SHP2. Such therapy may include radiotherapy. Such therapy may also comprise the administration of another pharmacologically active compound, or of two or more other pharmacologically active compounds, particularly compound(s) active in the prevention/treatment of cancer, also referred to as “anti-cancer drug(s)” or “chemotherapy agents”. For example, a compound of the invention, including any pharmaceutically acceptable salt, tautomer, solvate, or stereoisomer thereof as defined above, may be administered simultaneously, sequentially or separately in combination with any one or more other pharmacologically active compound. For simultaneous administration, the compound of the present invention and the other one or more pharmacologically active compound may be formulated in the same composition.

Classes of anti-cancer drugs that may be combined with the compounds of the invention include, but are not limited to: alkylating agents, anti-metabolites, antimitotics, checkpoint inhibitors, plant alkaloids and terpenoids, topoisomerase inhibitors, cytotoxic antibiotics, aromatase inhibitors, angiogenesis inhibitors, anti-steroids and anti-androgens, mTOR inhibitors, tyrosine kinase inhibitors, and others. Chemotherapy agents include, for example, mitotic inhibitors such as a taxane, a vinca alkaloid, paclitaxel, docetaxel, vincristine, vinblastine, vinorelbine or vinflunine, and other anticancer agents, e.g. cisplatin, 5-fluorouracil or 5-fluoro-2-4(1H,3H)-pyrimidinedione (5FU), flutamide or gemcitabine. Such combinations may offer significant advantages, including synergistic activity, in therapy.

Alkylating agents are compounds that work by adding an alkyl group to the guanine base of the DNA molecule, preventing the strands of the double helix from linking as they should thus causing breakage of the DNA strands and affecting the ability of the cancer cells to multiply. Antimetabolites are drugs that interfere with one or more enzymes or their reactions that are necessary for DNA synthesis. An antimitotic agent is a type of drug that blocks cell growth by stopping mitosis. Checkpoint inhibitors are a type of immunotherapy which block proteins that stop the immune system from attacking the cancer cells. Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle. Aromatase inhibitors are a class of drugs that work by inhibiting the action of the enzyme aromatase, which converts androgens into estrogens by a process called aromatization. Angiogenesis inhibitors are substances that inhibit the growth of new blood vessels and are used to treat cancers and other diseases that involve a proliferation of blood vessels. mTOR inhibitors are a class of drugs that inhibit the mammalian target of rapamycin (mTOR), which is a serine/threonine-specific protein kinase that belongs to the family of phosphatidylinositol-3 kinase (PI3K) related kinases (PIKKs). mTOR regulates cellular metabolism, growth, and proliferation by forming and signaling through two protein complexes, mTORC1 and mTORC2. The most established mTOR inhibitors are so-called rapalogs (rapamycin and its analogs), which have shown tumor responses in clinical trials against various tumor types.

It is thus a preferred object of the invention a compound as defined above or the pharmaceutically acceptable salt, tautomer, solvate, or stereoisomer thereof, for use in combination with at least one further therapeutic agent.

In any case, the multiple therapeutic agents (at least one of which is a compound disclosed herein) may be administered in any order or even simultaneously.

Preferably, said at least one further therapeutic agent is selected from the group consisting of:
(a) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, and cyclophosphamide (ENDOXAN);
(b) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX) and raltitrexed;
(c) antimitotics, which are often plant alkaloids and terpenoids, or derivatives thereof, including but not limited to taxanes such as docetaxel (TAXITERE) and paclitaxel (ABRAXANE, TAXOL); vinca alkaloids such as vincristine (ONCOVIN), vinblastine, vindesine, vinorelbine (NAVELBINE), and vinflunine;
(d) checkpoint inhibitors, such as anti-PD-1 or PD-L1 antibodies pembrolizumab (KEYTRUDA), nivolumab (OPDIVO), MEDI4736 and MPDL3280A; anti-CTLA-4 antibody ipilimumab (YERVOY); inhibitors that target LAG3 (lymphocyte activation gene 3 protein), KIR (killer cell immunoglobulin-like receptor), 4-1BB (tumour necrosis factor receptor superfamily member 9), TIM3 (T-cell immunoglobulin and mucin-domain containing-3) and/or OX40 (tumour necrosis factor receptor superfamily member 4);
(e) topoisomerase inhibitors, including but not limited to camptothecin (CTP), irinotecan (CAMPTOSAR), topotecan (HYCAMTIN), teniposide (VUMON) and etoposide (EPOSIN);
(f) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin; (7) aromatase inhibitors, including but not limited to aminoglutethimide, anastrozole (ARIMIDEX), letrozole (FEMARA), vorozole (RIVIZOR) and exemestane (AROMASIN);
(g) angiogenesis inhibitors, including but not limited to genistein, sunitinib (SUTENT) and bevacizumab (AVASTIN);
(h) anti-steroids and anti-androgens such as aminoglutethimide (CYTADREN), bicalutamide (CASODEX), cyproterone, flutamide (EULEXIN) and nilutamide (NILANDRON);
(i) tyrosine kinase inhibitors, including but not limited to imatinib (GLEEVEC), erlotinib (TARCEVA), lapatininb (TYKERB), sorafenib (NEXAVAR), and axitinib (INLYTA);
(j) mTOR inhibitors such as everolimus, temsirolimus (TORISEL), and sirolimus; (12) monoclonal antibodies such as trastuzumab (HERCEPTIN) and rituximab (RITUXAN);
(k) other agents, such as amsacrine; Bacillus Calmette-Guérin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; testosterone; 5-fluoro-2-4(1H,3H)-pyrimidinedione and combinations thereof.

The invention further provides pharmaceutical preparations comprising the compounds of the invention. In particular, it is a further object of the invention a pharmaceutical composition comprising the compound or the pharmaceutically acceptable salt, tautomer, solvate, or stereoisomer thereof as defined above, alone or in combination with at least one further therapeutic agent, and at least one pharmaceutically acceptable excipient. Preferably, said at least one further therapeutic agent in the pharmaceutical composition is selected among those indicated above.

In a preferred embodiment, the pharmaceutical combination or the composition of the invention is for use in the treatment and/or prevention of a disease or disorder as herein defined, in particular a disease or disorder mediated by the activity of SHP2 and/or a disease or disorder selected from the group consisting of: cancer, cardiovascular disease, immunological disorder, fibrosis, an ocular disorder, systemic lupus erythematosus, diabetes, neutropenia and combinations thereof.

The invention also provides pharmaceutical compositions comprising one or more compounds of this invention and a pharmaceutically acceptable carrier. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions of the invention may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water-soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soybean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound(s) of the invention are employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The compounds of the invention may be presented in a liposome or other micro particulate or other nanoparticle designed to target the compound. Acceptable liposomes can be neutral, negatively, or positively charged, the charge being a function of the charge of the liposome components and pH of the liposome solution. Liposomes can be normally prepared using a mixture of Phospholipids and cholesterol. Suitable phospholipids include phosphatidylcholine, phosphatidylethanolamine, phosphatidic acid, phosphotidylglycerol, phosphatidylinositol. Polyethylene glycol can be added to improve the blood circulation time of liposomes. Acceptable nanoparticles include albumin nanoparticles and gold nanoparticles.

When a compound according to this invention is administered into a human subject, the daily dosage will normally be determined by the prescribing physician with the dosage generally varying according to the age, weight, sex and response of the individual patient, as well as the severity of the patient's symptoms. Generally, dosage levels on the order of from about 0.01 mg/kg to about 500 mg/kg of body weight are useful in the treatment of the above indicated conditions. The dose range for adult humans is generally from 5 mg to 2 g/day.

As used herein, the term “composition” is intended to encompass a product comprising the specified ingredients in the specified amounts, as well as any product which results, directly or indirectly, from combination of the specified ingredients in the specified amounts.

Another object of the present invention relates to an in vitro method of inhibiting SHP2 with the compound of the present invention. This may be useful, for instance, to evaluate whether any given compound is an inhibitor/activator of SHP2 and therefore acts also on the ERK pathway.

A further object of the present invention concerns a kit comprising at least one pharmaceutically acceptable vial or container of other type, containing one or more doses of a compound of the invention, including any pharmaceutically acceptable salt, tautomer, solvate or stereoisomer thereof, or of a pharmaceutical composition of the invention and optionally a) instructions for use thereof in mammals and/or b) an infusion bag or container containing a pharmaceutically acceptable diluent.

In certain embodiments, the compound or the composition of the invention is administered parenterally, intramuscularly, intravenously, subcutaneously, orally, pulmonary, intrathecally, topically, intranasally, or systemically.

In certain embodiments, the patient who is administered the compound or the composition of the invention is a mammal, preferably a primate, more preferably a human.

The compounds of this invention may be administered to mammals, preferably humans, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

As used herein, the term “prevention” means no disorder or disease development if none had occurred, or no further disorder or disease development if there had already been development of the disorder or disease. Also considered is the ability of one to prevent some or all of the symptoms associated with the disorder or disease. As used herein, any reference to “treatment”/“treating” includes the amelioration of at least one symptom of the disease/disorder to be treated. Such amelioration is to be evaluated in comparison to the same symptom prior to administration of the compound or composition of the invention.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The present invention will be described by means of the following non-limiting examples and biological data.

### Materials and Methods

### Chemistry

As used herein, the following abbreviations have the following meanings. If an abbreviation is not defined, it has its generally accepted meaning.

### ABBREVIATIONS

AcOH: Acetic acid; AcCl: Acetyl chloride; Ac₂O: Acetic anhydride; AgNO₃: Silver nitrate; AISF: 4-(Acetylamino)phenyl]imidodisulfuryl difluoride; APS: Ammonium persulfate; B₂pin₂: Bis(pinacolato)diboron; BMS: borane dimethyl sulfide; (Boc)₂O: Di-tert-butyl dicarbonate; BOP: (Benzotriazol-1-yloxy)tris(dimethylamino)phosphonium hexafluorophosphate; 15-crown-5: 1,4,7,10,13-Pentaoxacyclopentadecane; n-BuLi: n-Butyllithium; 1-BuOH: 1-butanol; t-BuOH; t-butanol; tBuOK: Potassium tert-butoxide; CCl₄: carbon tetrachloride; CDI: 1,1′-Carbonyldiimidazole; CDCl₃: Deuterated chloroform; CHCl₃: Chloroform; CoCl₂: Cobalt(II) chloride; CuBr: Copper (I) bromide; CuI: Copper iodide; DABSO: 1,4-Diazabicyclo [2.2.2]octane bis (sulfur dioxide); DAST: Diethylaminosulfur trifluoride; DBU: 1,8-Diazabicyclo[5.4.0]undec-7-ene; DCE: Dichloroethane; DCM: Dichloromethane; DEA: Diethanol amine; DHP: 3,4-Dihydro-2H-pyran; DIAD: Diisopropyl azodicarboxylate; DIBAL-H: Diisobutylaluminium hydride; DIPEA: N,N-Diisopropylethylamine; DMA: N,N-Dimethylacetamide; DMAP:4-(Dimethylamino)pyridine; DME: Dimethoxyethane; DMEDA: 1,2-Dimethylethylenediamine; DMSO: Dimethylsulfoxide; EDC-HCl: 1-Ethyl-3-(3′-dimethylaminopropyl)carbodiimide HCl; ES⁺: Electrospray Positive Ionisation; Et₂O: Diethylether; EtOAc: Ethyl acetate; EtOH: Ethanol; Fe: iron; HATU: (1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium; HBF₄: Tetrafluoroboric acid; HCl: hydrochloric acid; h: Hour; H₂: Hydrogen; H₂O: Water; HPLC: High Performance Liquid Chromatography; H₂SO₄: Sulfuric acid; HBTU: 2-(1H-benzotriazol-1-yl)-1,1,3,3-tetramethyluronium hexafluorophosphate; IPA: 2-Propanol; KHF₂: Potassium bifluoride KI: Potassium iodide; KOAc: Potassium acetate; K₃PO₄: Tripotassium phosphate; K₂CO₃: potassium carbonate; LiAlH₄: Lithium aluminium hydride; LCMS: Liquid Chromatography Mass Spectrometry; LDA: Lithium diisopropylamide; LiCl: Lithium chloride; LiOH: Lithium hydroxide; mCPBA: meta-Chloroperoxybenzoic acid; MeCN: Acetonitrile; Mel: Iodomethane; MeOH: Methanol; min: Minutes; MnO₂: Manganese dioxide; MW: Microwave; N₂: nitrogen; NaH: Sodium hydride; NaNO₂: Sodium nitrite; NaOH: Sodium hydroxide; NaOMe: Sodium methoxide; NaOtBu: Sodium tert-butoxide; NaBH₄: sodium borohydride; Na₂CO₃: Sodium carbonate; NaHCO₃: Sodium bicarbonate; NaN₃: sodium azide; Na₂SO₄: sodium sulfate; NBS: N-bromosuccinimide; NFSI: N-Fluorobenzenesulfonimide; NH₄Cl: ammonium chloride; NH₂OH·HCl: Hydroxylamine hydrochloride; NH₄OH: Ammonium hydroxide; NIS: N-Iodosuccinimide; N₂H₄: Hydrazine; NMM: N-Methylmorpholine; NMP: N-Methyl-2-pyrrolidone; Pd₂(dba)₃: Tris(dibenzylideneacetone)dipalladium(0); Pd₂(dba)₃: Tris(dibenzylideneacetone)dipalladium(0) Pd(dppf)Cl₂·DCM: [1,1′-Bis(diphenylphosphino)ferrocene]dichloropalladium(II) complex with DCM; Pd(PPh₃)₄: Tetrakis(triphenylphosphine)palladium(0); Pd(OAc)₂: Palladium(II) acetate; Pd(PPh₃)₂Cl₂: Bis(triphenylphosphine)palladium(II) dichloride; POCl₃: Phosphoryl chloride; PPh₃: Triphenylphosphine; PTSA: p-Toluenesulfonic acid; RP: Reverse Phase; RT: Retention time; rt: Room temperature; RuPhos: dicyclohexyl(2′,6′-diisopropoxybiphenyl-2-yl)phosphine; Sat.: Saturated; SAX: Strong Anion Exchanger; Selectfluor: 1-Chloromethyl-4-fluoro-1,4-diazoniabicyclo[2.2.2]octane bis(tetrafluoroborate); SEM: trimethylsilylethoxymethyl; Sol.: Solution; SnCl₂: Tin (II) chloride; TBAF: Tetrabutylammonium fluoride; TBDMS-Cl: tert-Butyldimethylsilyl chloride; TCDI: 1-1′-Thiocarbonyldiimidazole; TEA: Triethylamine; [(t-Bu)₃PH]BF₄: Tri-tert-butylphosphonium tetrafluoroborate; TFA: Trifluoroacetic acid; THF: Tetrahydrofuran; THP: Tetrahydropyranyl; Sat.: Saturated; Sol.: Solution; UPLC: Ultra High Performance Liquid Chromatography; SFC (Supercritical fluid chromatography); XantPhos: 4,5-Bis(diphenylphosphino)-9,9-dimethylxanthene; Xphos: 2-Dicyclohexylphosphino-2′,4′,6′-triisopropylbiphenyl

### General

Solvents and reagents were obtained from commercial suppliers and were used without further purification. Flash chromatography purifications were performed on prepacked cartridges on a Biotage system. Purity of final compounds was determined using MS and UPLC. UPLC-MS analyses were performed on a Waters Acquity UPLC™, equipped with a diode array and a ZQ mass spectrometer, using column 1: BEH C18 column (1.7 mm, 2.1×50 mm), column 2: BEH C18 column (1.7 mm, 2.1×100 mm); Mobile phase containing H₂O (HiPerSolv Chromanorm Water VWR for HPLC-MS)+0.1% formic acid (A), and MeCN (HiPerSolv Chromanorm Acetonitrile SuperGradient VWR, suitable for UPLC/UHPLC instruments)+0.1% formic acid (B) was used in the reported methods:

Method 1: column 1, run time: 3 min, run gradient: (A): 70% (0.1 min), 70%-50% (2.6 min), 50%-50% (0.3 min), 0% (0.1 min) with a 0.5 mL/min flow.

Method 2: column 1, run time: 3 min, run gradient: (A): 90% (0.1 min), 90%-0% (2.6 min), 0% (0.3 min), 0%-90% (0.1 min) with a 0.5 mL/min flow.

Method 3: column 1, run time: 3 min, run gradient: (A): 80% (0.1 min), 80%-60% (2.6 min), 60%-60% (0.3 min), 0% (0.1 min) with a 0.5 mL/min flow.

Mobile phase containing H₂O (HiPerSolv Chromanorm Water VWR for HPLC-MS)+0.05% Trifluoroacetic Acid (A), and MeCN (HiPerSolv Chromanorm Acetonitrile SuperGradient VWR, suitable for UPLC/UHPLC instruments)+0.05% Trifluoroacetic Acid (B) was used in the reported methods: Method 1A: column 1, run time: 3 min, run gradient: 5% B to 100% B in 2.80 min+100% B for 0.2 min, with a 0.5 mL/min flow.

Method 2A: column 2, run time: 6 min, run gradient: 5% B to 100% B in 5 min+100% B for 1 min, with a 0.5 mL/min flow.

Method 3A: column 1, run time: 3 min, run gradient: 40% B to 100% B in 2.80 min+100% B for 0.2 min, with a 0.5 mL/min flow.

Method 4A: column 1, run time: 6 min, run gradient: 5% B to 100% B in 5 min+100% B for 1 min, with a 0.5 mL/min flow.

Purity of final compounds was >95%. All ¹H spectra were recorded on Bruker AV400 spectrometer at 400 MHz except where indicated. Chemical shifts (δ) are reported in parts per million relative to TMS using CDCl₃ as a solvent or relative to the residual solvent signal using DMSO-d₆. Coupling constants (J) are reported in Hertz (Hz). Multiplicities are reported as singlet (s), broad (br), doublet (d), doublet of doublet (dd), doublet of doublet of doublet (ddd), triplet (t), doublet of triplet (dt), doublet of doublet of triplet (ddt), triplet of triplet (tt), quartet (q), doublet of quartets (dq) or multiplet (m). Unless indicated, spectra were acquired at 300 K. Temperatures are expressed in degrees Celsius (° C.) and are uncorrected.

### Processes for Making the Compounds of the Invention

The present invention also includes processes for the preparation of compounds of the invention. The following schemes are examples of synthetic routes that may be adopted to prepare compounds of the invention.

In the reaction schemes described below, it can be useful to protect reactive functional groups, for example amino, imino, hydroxyl, thio or carboxy groups, to avoid their unwanted participations to reactions. Conventional protecting groups can be used in accordance with standard practice, for example see T. Greene and P.G. M. Wuts in “Protective Groups in Organic Chemistry”, John Wiley and Sons, 1991.

Those skilled in the art will readily appreciate that certain compounds of the invention can be converted into other compounds of the invention according to standard chemical methods (e.g. by salification/desalification).

The following examples were synthesized using according to the Schemes and the procedures described infra or modifications thereof using the appropriate starting materials (Table 1).

TABLE 1
Compounds prepared according to the Examples and experimental data (MS).

| Ex- | | | MS |
|---|---|---|---|
| ample | Chemical Structure | IUPAC Name | [M + H]+ |
| 1 | 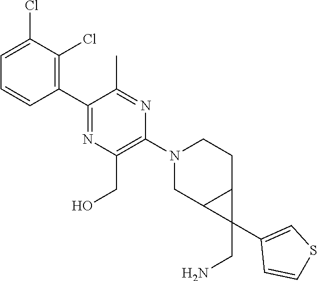 [SMILES: Cc1nc(N2CCC3C(C2)C3(CN)c2ccsc2)c(CO)nc1-c1cccc(Cl)c1Cl] | (3-(7-(aminomethyl)-7-(thiophen-3-yl)-3- azabicyclo[4.1.0]heptan-3-yl)-6-(2,3- dichlorophenyl)-5-methylpyrazin-2-yl)methanol | 475.44 |
| 2 | 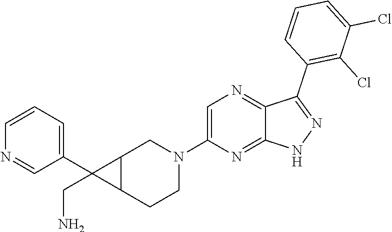 [SMILES: NCC1(c2cccnc2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(pyridin-3-yl)-3- azabicyclo[4.1.0]heptan-7-yl)methanamine | 466.37 |
| 3 | 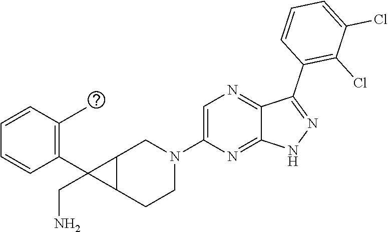 [SMILES: Cc1ccccc1C1(CN)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(2-fluorophenyl)-3- azabicyclo[4.1.0]heptan-7-yl)methanamine | 483.37 |
| 4 | 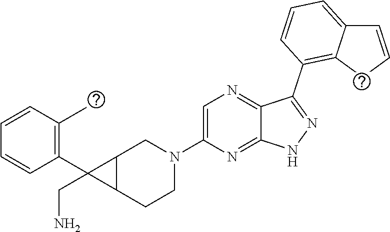 [SMILES: Cc1ccccc1C1(CN)C2CCN(c3cnc4c(-c5cccc6c5CC=C6)n[nH]c4n3)CC21] | (3-(3-(benzo[b]thiophen-7-yl)-1H-pyrazolo[3,4- b]pyrazin-6-yl)-7-(2-fluorophenyl)-3- azabicyclo[4.1.0]heptan-7-yl)methanamine | 470.57 |
| 5 | 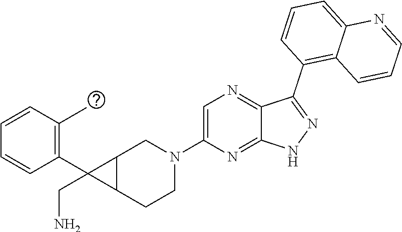 [SMILES: Cc1ccccc1C1(CN)C2CCN(c3cnc4c(-c5cccc6ncccc56)n[nH]c4n3)CC21] | (7-(2-fluorophenyl)-3-(3-(quinolin-5-yl)-1H- pyrazolo[3,4-b]pyrazin-6-yl)-3- azabicyclo[4.1.0]heptan-7-yl)methanamine | 465.53 |
| 6 | 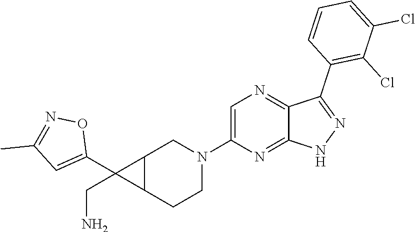 [SMILES: Cc1cc(C2(CN)C3CCN(c4cnc5c(-c6cccc(Cl)c6Cl)n[nH]c5n4)CC32)on1] | (3-(3-(2,3-dichlorophenyl)-1H-pyrazolo[3,4- b]pyrazin-6-yl)-7-(3-methylisoxazol-5-yl)-3- azabicyclo[4.1.0]heptan-7-yl)methanamine | 470.36 |
| 7 | 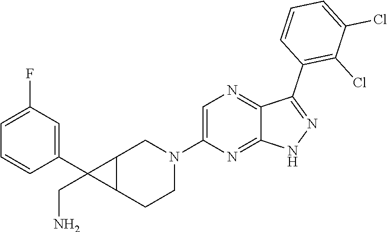 [SMILES: NCC1(c2cccc(F)c2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(3-fluorophenyl)-3- azabicyclo[4.1.0]heptan-7-yl)methanamine | 483.37 |
| 8 | 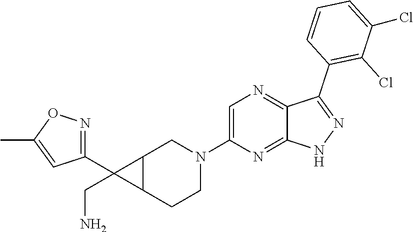 [SMILES: Cc1cc(C2(CN)C3CCN(c4cnc5c(-c6cccc(Cl)c6Cl)n[nH]c5n4)CC32)no1] | (3-(3-(2,3-dichlorophenyl)-1H-pyrazolo[3,4- b]pyrazin-6-yl)-7-(5-methylisoxazol-3-yl)-3- azabicyclo[4.1.0]heptan-7-yl)methanamine | 470.36 |
| 9 | 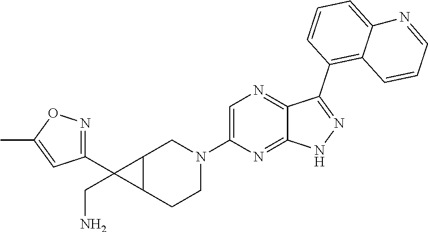 [SMILES: Cc1cc(C2(CN)C3CCN(c4cnc5c(-c6cccc7ncccc67)n[nH]c5n4)CC32)no1] | (7-(5-methylisoxazol-3-yl)-3-(3-(quinolin-5-yl)-1H- pyrazolo[3,4-b]pyrazin-6-yl)-3- azabicyclo[4.1.0]heptan-7-yl)methanamine | 452.51 |
| 10 | 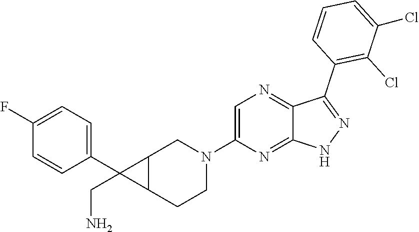 [SMILES: NCC1(c2ccc(F)cc2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(4-fluorophenyl)-3- azabicyclo[4.1.0]heptan-7-yl)methanamine | 483.37 |
| 11 | 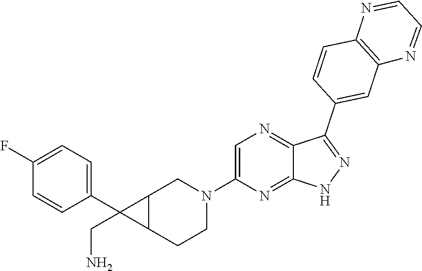 [SMILES: NCC1(c2ccc(F)cc2)C2CCN(c3cnc4c(-c5ccc6nccnc6c5)n[nH]c4n3)CC21] | (7-(4-fluorophenyl)-3-(3-(quinoxalin-6-yl)-1H- pyrazolo[3,4-b]pyrazin-6-yl)-3- azabicyclo[4.1.0]heptan-7-yl)methanamine | 466.51 |
| 12 | 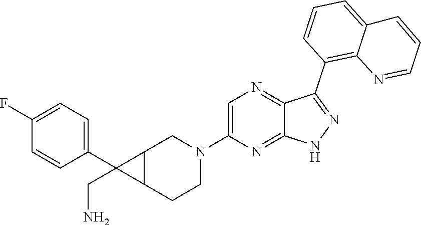 [SMILES: NCC1(c2ccc(F)cc2)C2CCN(c3cnc4c(-c5cccc6cccnc56)n[nH]c4n3)CC21] | (7-(4-fluorophenyl)-3-(3-(quinolin-8-yl)-1H- pyrazolo[3,4-b]pyrazin-6-yl)-3- azabicyclo[4.1.0]heptan-7-yl)methanamine | 465.53 |
| 13 | 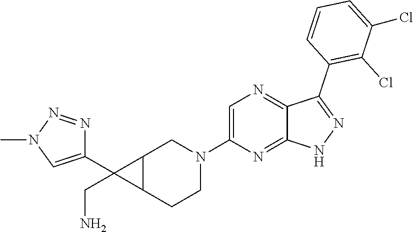 [SMILES: Cn1cc(C2(CN)C3CCN(c4cnc5c(-c6cccc(Cl)c6Cl)n[nH]c5n4)CC32)nn1] | (3-(3-(2,3-dichlorophenyl)-1H-pyrazolo[3,4- b]pyrazin-6-yl)-7-(1-methyl-1H-1,2,3-triazol-4-yl)-3- azabicyclo[4.1.0]heptan-7-yl)methanamine | 470.36 |
| 14 | 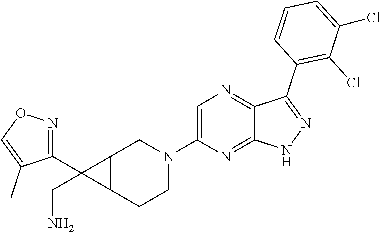 [SMILES: Cc1conc1C1(CN)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(4-methylisoxazol-3-yl)-3- azabicyclo[4.1.0]heptan-7-yl)methanamine | 470.36 |
| 15 | 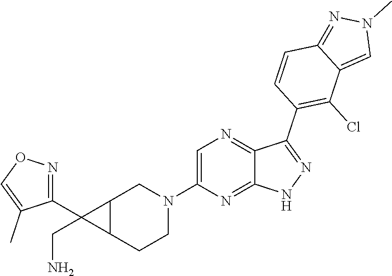 [SMILES: Cc1conc1C1(CN)C2CCN(c3cnc4c(-c5ccc6nn(C)cc6c5Cl)n[nH]c4n3)CC21] | (3-(3-(4-chloro-2-methyl-2H-indazol-5-yl)-1H- pyrazolo[3,4-b]pyrazin-6-yl)-7-(4-methylisoxazol-3- yl)-3-azabicyclo[4.1.0]heptan-7-yl)methanamine | 489.96 |
| 16 | 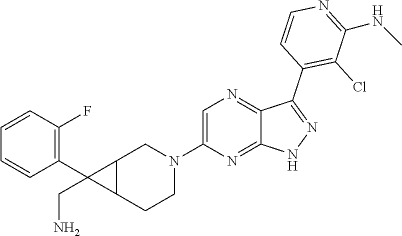 [SMILES: CNc1nccc(-c2n[nH]c3nc(N4CCC5C(C4)C5(CN)c4ccccc4F)cnc23)c1Cl] | 4-(6-(7-(aminomethyl)-7-(2-fluorophenyl)-3- azabicyclo[4.1.0]heptan-3-yl)-1H-pyrazolo[3,4- b]pyrazin-3-yl)-3-chloro-N-methylpyridin-2-amine | 478.95 |
| 17 | 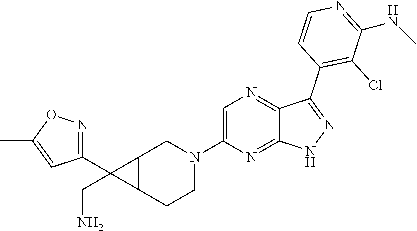 | 4-(6-(7-(aminomethyl)-7-(5-methylisoxazol-3-yl)-3- azabicyclo[4.1.0]heptan-3-yl)-1H-pyrazolo[3,4- b]pyrazin-3-yl)-3-chloro-N-methylpyridin-2-amine | 465.94 |
| 18 | 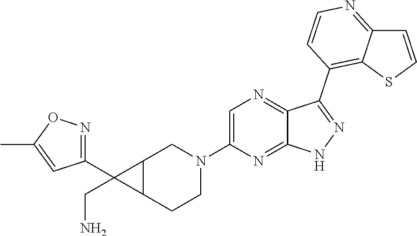 [SMILES: Cc1cc(C2(CN)C3CCN(c4cnc5c(-c6ccnc7ccsc67)n[nH]c5n4)CC32)no1] | (7-(5-methylisoxazol-3-yl)-3-(3-(thieno[3,2- b]pyridin-7-yl)-1H-pyrazolo[3,4-b]pyrazin-6-yl)-3- azabicyclo[4.1.0]heptan-7-yl)methanamine | 458.54 |
| 19 | 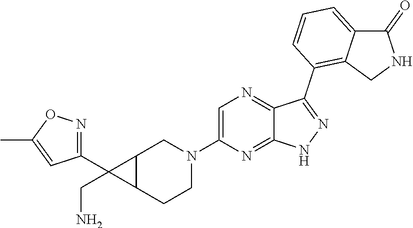 [SMILES: Cc1cc(C2(CN)C3CCN(c4cnc5c(-c6cccc7c6CNC7=O)n[nH]c5n4)CC32)no1] | 4-(6-(7-(aminomethyl)-7-(5-methylisoxazol-3-yl)-3- azabicyclo[4.1.0]heptan-3-yl)-1H-pyrazolo[3,4- b]pyrazin-3-yl)isoindolin-1-one | 456.5 |
| 20 | 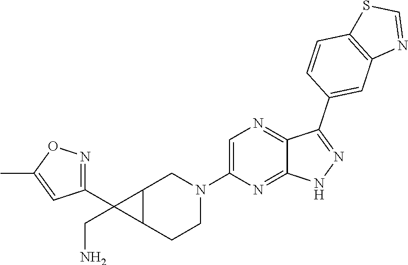 [SMILES: Cc1cc(C2(CN)C3CCN(c4cnc5c(-c6ccc7scnc7c6)n[nH]c5n4)CC32)no1] | (3-(3-(benzo[d]thiazol-5-yl)-1H-pyrazolo[3,4- b]pyrazin-6-yl)-7-(5-methylisoxazol-3-yl)-3- azabicyclo[4.1.0]heptan-7-yl)methanamine | 458.54 |
| 21 | 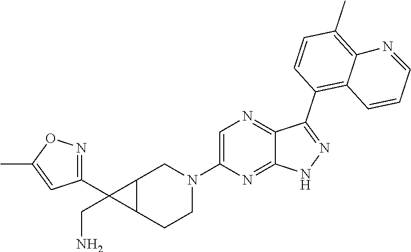 [SMILES: Cc1cc(C2(CN)C3CCN(c4cnc5c(-c6ccc(C)c7ncccc67)n[nH]c5n4)CC32)no1] | (7-(5-methylisoxazol-3-yl)-3-(3-(8-methylquinolin-5- yl)-1H-pyrazolo[3,4-b]pyrazin-6-yl)-3- azabicyclo[4.1.0]heptan-7-yl)methanamine | 466.54 |
| 22 | 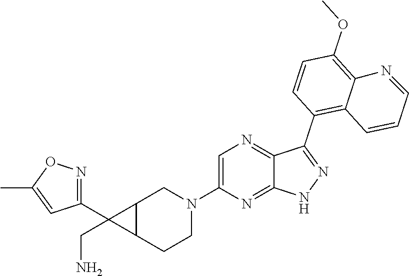 [SMILES: COc1ccc(-c2n[nH]c3nc(N4CCC5C(C4)C5(CN)c4cc(C)on4)cnc23)c2cccnc12] | (3-(3-(8-methoxyquinolin-5-yl)-1H-pyrazolo[3,4- b]pyrazin-6-yl)-7-(5-methylisoxazol-3-yl)-3- azabicyclo[4.1.0]heptan-7-yl)methanamine | 482.54 |
| 23 | 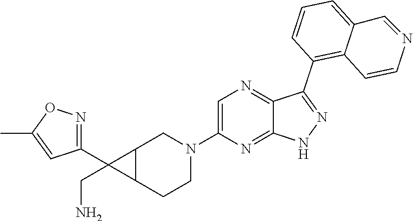 [SMILES: Cc1cc(C2(CN)C3CCN(c4cnc5c(-c6cccc7cnccc67)n[nH]c5n4)CC32)no1] | (3-(3-(isoquinolin-5-yl)-1H-pyrazolo[3,4-b]pyrazin- 6-yl)-7-(5-methylisoxazol-3-yl)-3- azabicyclo[4.1.0]heptan-7-yl)methanamine | 452.51 |
| 24 | 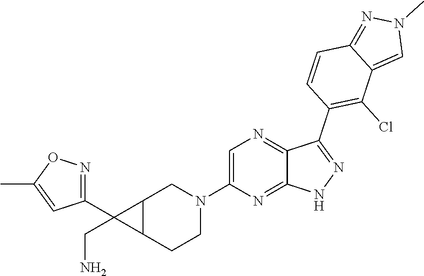 | (3-(3-(4-chloro-2-methyl-2H-indazol-5-yl)-1H- pyrazolo[3,4-b]pyrazin-6-yl)-7-(5-methylisoxazol-3- yl)-3-azabicyclo[4.1.0]heptan-7-yl)methanamine | 489.96 |
| 25 | 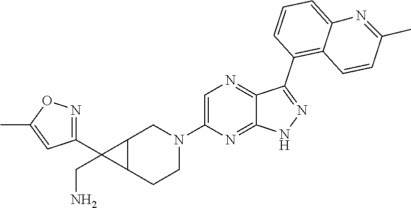 | (7-(5-methylisoxazol-3-yl)-3-(3-(2-methylquinolin-5- yl)-1H-pyrazolo[3,4-b]pyrazin-6-yl)-3- azabicyclo[4.1.0]heptan-7-yl)methanamine | 466.54 |
| 26 | 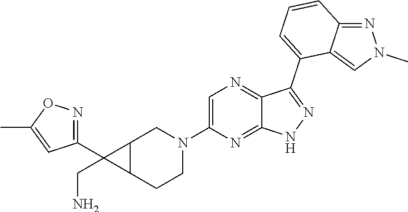 | (3-(3-(2-methyl-2H-indazol-4-yl)-1H-pyrazolo[3,4- b]pyrazin-6-yl)-7-(5-methylisoxazol-3-yl)-3- azabicyclo[4.1.0]heptan-7-yl)methanamine | 455.52 |
| 27 | 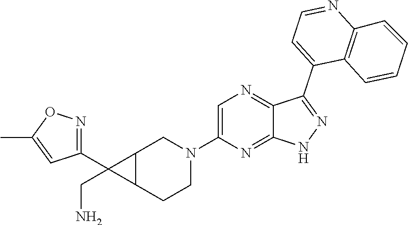 [SMILES: Cc1cc(C2(CN)C3CCN(c4cnc5c(-c6ccnc7ccccc67)n[nH]c5n4)CC32)no1] | (7-(5-methylisoxazol-3-yl)-3-(3-(quinolin-4-yl)-1H- pyrazolo[3,4-b]pyrazin-6-yl)-3- azabicyclo[4.1.0]heptan-7-yl)methanamine | 452.51 |
| 28 | 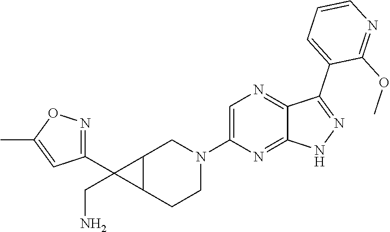 [SMILES: COc1ncccc1-c1n[nH]c2nc(N3CCC4C(C3)C4(CN)c3cc(C)on3)cnc12] | (3-(3-(2-methoxypyridin-3-yl)-1H-pyrazolo[3,4- b]pyrazin-6-yl)-7-(5-methylisoxazol-3-yl)-3- azabicyclo[4.1.0]heptan-7-yl)methanamine | 432.48 |
| 29 | 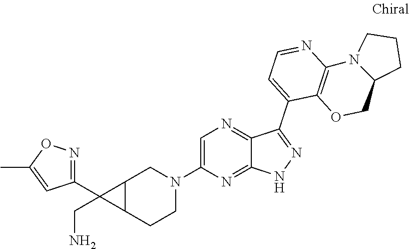 [SMILES: Cc1cc(C2(CN)C3CCN(c4cnc5c(-c6ccnc7c6OC[C@@H]6CCCN76)n[nH]c5n4)CC32)no1] | (7-(5-methylisoxazol-3-yl)-3-(3-((S)-6a,7,8,9- tetrahydro-6H-pyrido[3,2-b]pyrrolo[1,2- d][1,4]oxazin-4-yl)-1H-pyrazolo[3,4-b]pyrazin-6-yl)- 3-azabicyclo[4.1.0]heptan-7-yl)methanamine | 499.57 |
| 30 | 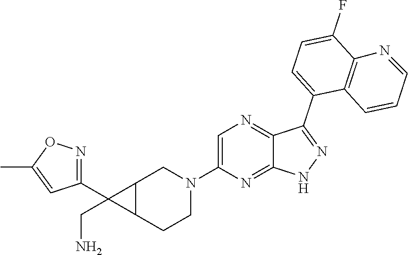 [SMILES: Cc1cc(C2(CN)C3CCN(c4cnc5c(-c6ccc(F)c7ncccc67)n[nH]c5n4)CC32)no1] | (3-(3-(8-fluoroquinolin-5-yl)-1H-pyrazolo[3,4- b]pyrazin-6-yl)-7-(5-methylisoxazol-3-yl)-3- azabicyclo[4.1.0]heptan-7-yl)methanamine | 470.5 |
| 31 | 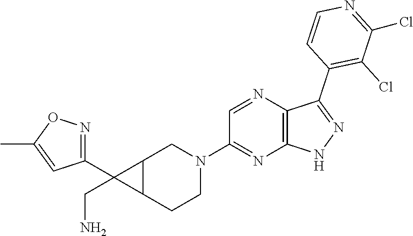 [SMILES: Cc1cc(C2(CN)C3CCN(c4cnc5c(-c6ccnc(Cl)c6Cl)n[nH]c5n4)CC32)no1] | (3-(3-(2,3-dichloropyridin-4-yl)-1H-pyrazolo[3,4- b]pyrazin-6-yl)-7-(5-methylisoxazol-3-yl)-3- azabicyclo[4.1.0]heptan-7-yl)methanamine | 471.34 |
| 32 | 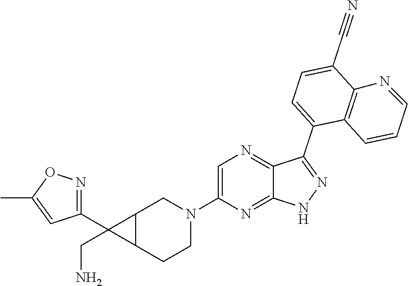 [SMILES: Cc1cc(C2(CN)C3CCN(c4cnc5c(-c6ccc(C#N)c7ncccc67)n[nH]c5n4)CC32)no1] | 5-(6-(7-(aminomethyl)-7-(5-methylisoxazol-3-yl)-3- azabicyclo[4.1.0]heptan-3-yl)-1H-pyrazolo[3,4- b]pyrazin-3-yl)quinoline-8-carbonitrile | 477.52 |
| 33 | 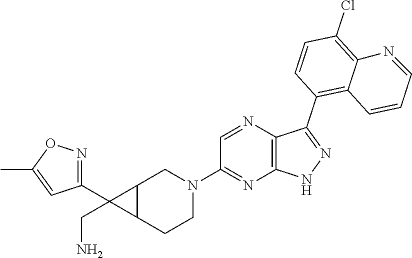 [SMILES: Cc1cc(C2(CN)C3CCN(c4cnc5c(-c6ccc(Cl)c7ncccc67)n[nH]c5n4)CC32)no1] | (3-(3-(8-chloroquinolin-5-yl)-1H-pyrazolo[3,4- b]pyrazin-6-yl)-7-(5-methylisoxazol-3-yl)-3- azabicyclo[4.1.0]heptan-7-yl)methanamine | 486.96 |
| 34 | 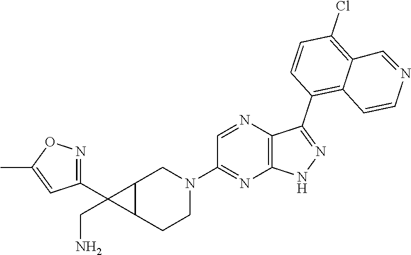 [SMILES: Cc1cc(C2(CN)C3CCN(c4cnc5c(-c6ccc(Cl)c7cnccc67)n[nH]c5n4)CC32)no1] | (3-(3-(8-chloroisoquinolin-5-yl)-1H-pyrazolo[3,4- b]pyrazin-6-yl)-7-(5-methylisoxazol-3-yl)-3- azabicyclo[4.1.0]heptan-7-yl)methanamine | 486.96 |
| 35 | 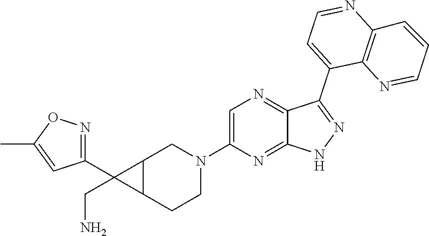 [SMILES: Cc1cc(C2(CN)C3CCN(c4cnc5c(-c6ccnc7cccnc67)n[nH]c5n4)CC32)no1] | (3-(3-(1,5-naphthyridin-4-yl)-1H-pyrazolo[3,4- b]pyrazin-6-yl)-7-(5-methylisoxazol-3-yl)-3- azabicyclo[4.1.0]heptan-7-yl)methanamine | 453.5 |
| 36 | 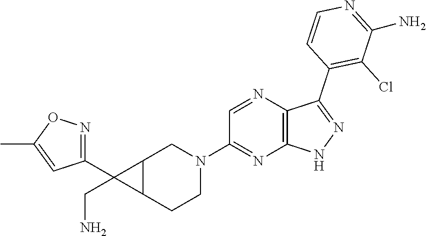 [SMILES: Cc1cc(C2(CN)C3CCN(c4cnc5c(-c6ccnc(N)c6Cl)n[nH]c5n4)CC32)no1] | 4-(6-(7-(aminomethyl)-7-(5-methylisoxazol-3-yl)-3- azabicyclo[4.1.0]heptan-3-yl)-1H-pyrazolo[3,4- b]pyrazin-3-yl)-3-chloropyridin-2-amine | 451.91 |
| 37 | 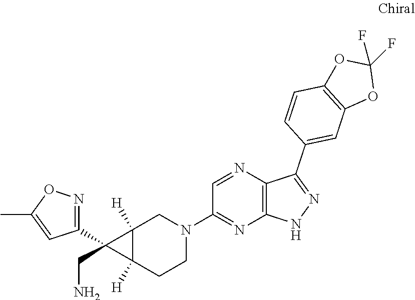 [SMILES: Cc1cc([C@@]2(CN)[C@@H]3CCN(c4cnc5c(-c6ccc7c(c6)OC(F)(F)O7)n[nH]c5n4)C[C@@H]32)no1] | ((1S,6R,7S)-3-(3-(2,2-difluorobenzo[d][1,3]dioxol-5- yl)-1H-pyrazolo[3,4-b]pyrazin-6-yl)-7-(5- methylisoxazol-3-yl)-3-azabicyclo[4.1.0]heptan-7- yl)methanamine | 481.46 |
| 38 | 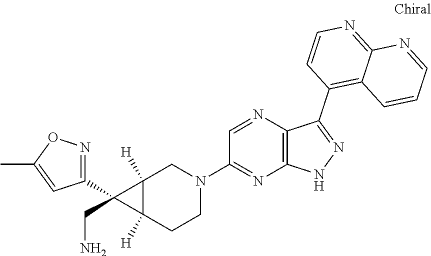 [SMILES: Cc1cc([C@@]2(CN)[C@@H]3CCN(c4cnc5c(-c6ccnc7ncccc67)n[nH]c5n4)C[C@@H]32)no1] | ((1S,6R,7S)-3-(3-(1,8-naphthyridin-4-yl)-1H- pyrazolo[3,4-b]pyrazin-6-yl)-7-(5-methylisoxazol-3- yl)-3-azabicyclo[4.1.0]heptan-7-yl)methanamine | 453.5 |
| 39 | 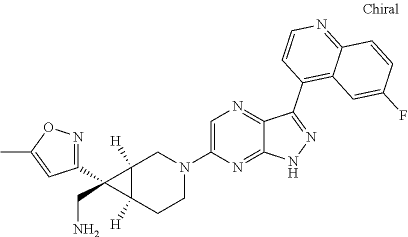 [SMILES: Cc1cc([C@@]2(CN)[C@@H]3CCN(c4cnc5c(-c6ccnc7ccc(F)cc67)n[nH]c5n4)C[C@@H]32)no1] | ((1S,6R,7S)-3-(3-(6-fluoroquinolin-4-yl)-1H- pyrazolo[3,4-b]pyrazin-6-yl)-7-(5-methylisoxazol-3- yl)-3-azabicyclo[4.1.0]heptan-7-yl)methanamine | 470.5 |
| 40 | 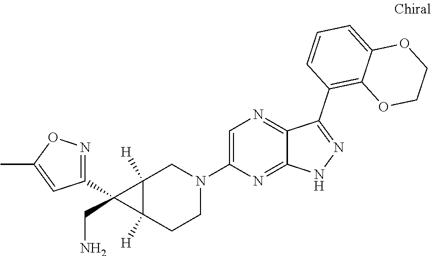 [SMILES: Cc1cc([C@@]2(CN)[C@@H]3CCN(c4cnc5c(-c6cccc7c6OCCO7)n[nH]c5n4)C[C@@H]32)no1] | ((1S,6R,7S)-3-(3-(2,3-dihydrobenzo[b][1,4]dioxin-5- yl)-1H-pyrazolo[3,4-b]pyrazin-6-yl)-7-(5- methylisoxazol-3-yl)-3-azabicyclo[4.1.0]heptan-7- yl)methanamine | 459.5 |
| 41 | 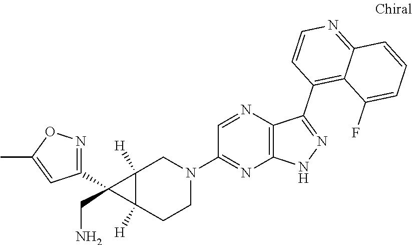 [SMILES: Cc1cc([C@@]2(CN)[C@@H]3CCN(c4cnc5c(-c6ccnc7cccc(F)c67)n[nH]c5n4)C[C@@H]32)no1] | ((1S,6R,7S)-3-(3-(5-fluoroquinolin-4-yl)-1H- pyrazolo[3,4-b]pyrazin-6-yl)-7-(5-methylisoxazol-3- yl)-3-azabicyclo[4.1.0]heptan-7-yl)methanamine | 470.5 |
| 42 | 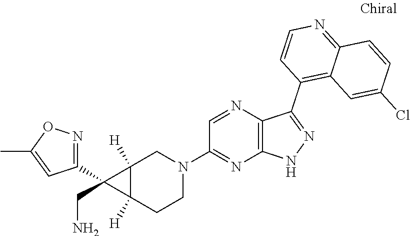 [SMILES: Cc1cc([C@@]2(CN)[C@@H]3CCN(c4cnc5c(-c6ccnc7ccc(Cl)cc67)n[nH]c5n4)C[C@@H]32)no1] | ((1S,6R,7S)-3-(3-(6-chloroquinolin-4-yl)-1H- pyrazolo[3,4-b]pyrazin-6-yl)-7-(5-methylisoxazol-3- yl)-3-azabicyclo[4.1.0]heptan-7-yl)methanamine | 486.96 |
| 43 | 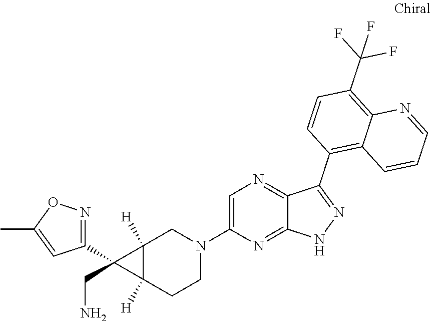 [SMILES: Cc1cc([C@@]2(CN)[C@@H]3CCN(c4cnc5c(-c6ccc(C(F)(F)F)c7ncccc67)n[nH]c5n4)C[C@@H]32)no1] | ((1S,6R,7S)-7-(5-methylisoxazol-3-yl)-3-(3-(8- (trifluoromethyl)quinolin-5-yl)-1H-pyrazolo[3,4- b]pyrazin-6-yl)-3-azabicyclo[4.1.0]heptan-7- yl)methanamine | 520.51 |
| 44 | 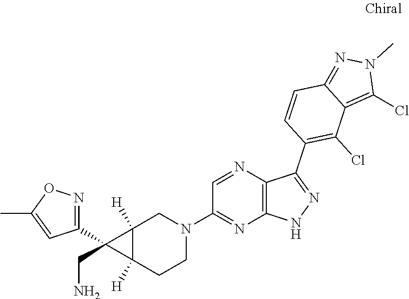 [SMILES: Cc1cc([C@@]2(CN)[C@@H]3CCN(c4cnc5c(-c6ccc7nn(C)c(Cl)c7c6Cl)n[nH]c5n4)C[C@@H]32)no1] | ((1S,6R,7S)-3-(3-(3,4-dichloro-2-methyl-2H-indazol- 5-yl)-1H-pyrazolo[3,4-b]pyrazin-6-yl)-7-(5- methylisoxazol-3-yl)-3-azabicyclo[4.1.0]heptan-7- yl)methanamine | 524.41 |
| 45 | 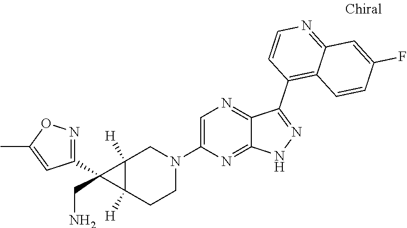 [SMILES: Cc1cc([C@@]2(CN)[C@@H]3CCN(c4cnc5c(-c6ccnc7cc(F)ccc67)n[nH]c5n4)C[C@@H]32)no1] | ((1S,6R,7S)-3-(3-(7-fluoroquinolin-4-yl)-1H- pyrazolo[3,4-b]pyrazin-6-yl)-7-(5-methylisoxazol-3- yl)-3-azabicyclo[4.1.0]heptan-7-yl)methanamine | 470.5 |
| 46 | 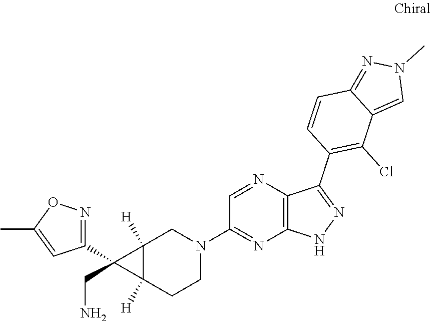 | ((1S,6R,7S)-3-(3-(4-chloro-2-methyl-2H-indazol-5- yl)-1H-pyrazolo[3,4-b]pyrazin-6-yl)-7-(5- methylisoxazol-3-yl)-3-azabicyclo[4.1.0]heptan-7- yl)methanamine | 489.96 |
| 47 | 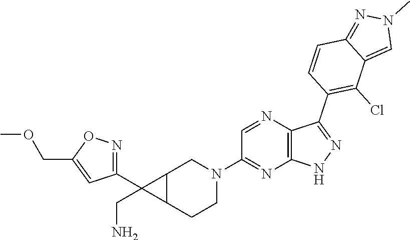 | (3-(3-(4-chloro-2-methyl-2H-indazol-5-yl)-1H- pyrazolo[3,4-b]pyrazin-6-yl)-7-(5- (methoxymethyl)isoxazol-3-yl)-3- azabicyclo[4.1.0]heptan-7-yl)methanamine | 519.99 |
| 48 | 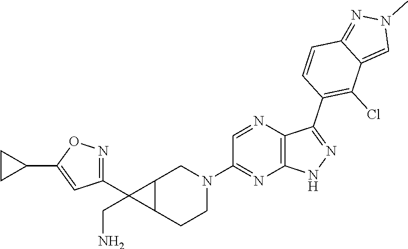 [SMILES: Cn1cc2c(Cl)c(-c3n[nH]c4nc(N5CCC6C(C5)C6(CN)c5cc(C6CC6)on5)cnc34)ccc2n1] | (3-(3-(4-chloro-2-methyl-2H-indazol-5-yl)-1H- pyrazolo[3,4-b]pyrazin-6-yl)-7-(5- cyclopropylisoxazol-3-yl)-3-azabicyclo[4.1.0]heptan- 7-yl)methanamine | 516 |
| 49 | 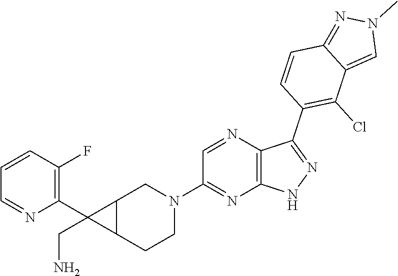 [SMILES: Cn1cc2c(Cl)c(-c3n[nH]c4nc(N5CCC6C(C5)C6(CN)c5ncccc5F)cnc34)ccc2n1] | (3-(3-(4-chloro-2-methyl-2H-indazol-5-yl)-1H- pyrazolo[3,4-b]pyrazin-6-yl)-7-(3-fluoropyridin-2- yl)-3-azabicyclo[4.1.0]heptan-7-yl)methanamine | 503.96 |
| 50 | 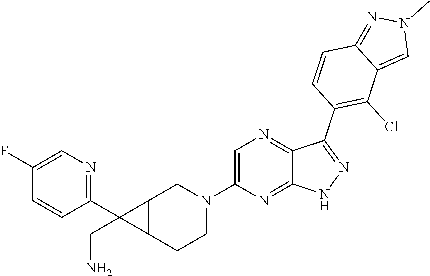 [SMILES: Cn1cc2c(Cl)c(-c3n[nH]c4nc(N5CCC6C(C5)C6(CN)c5ccc(F)cn5)cnc34)ccc2n1] | (3-(3-(4-chloro-2-methyl-2H-indazol-5-yl)-1H- pyrazolo[3,4-b]pyrazin-6-yl)-7-(5-fluoropyridin-2- yl)-3-azabicyclo[4.1.0]heptan-7-yl)methanamine | 503.96 |
| 51 | 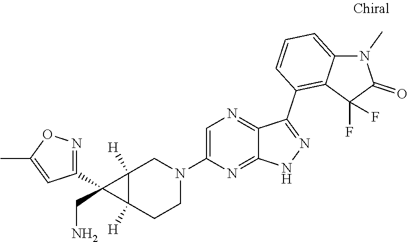 [SMILES: Cc1cc([C@@]2(CN)[C@@H]3CCN(c4cnc5c(-c6cccc7c6C(F)(F)C(=O)N7C)n[nH]c5n4)C[C@@H]32)no1] | 4-(6-((1S,6R,7S)-7-(aminomethyl)-7-(5- methylisoxazol-3-yl)-3-azabicyclo[4.1.0]heptan-3- yl)-1H-pyrazolo[3,4-b]pyrazin-3-yl)-3,3-difluoro-1- methylindolin-2-one | 506.51 |
| 52 | 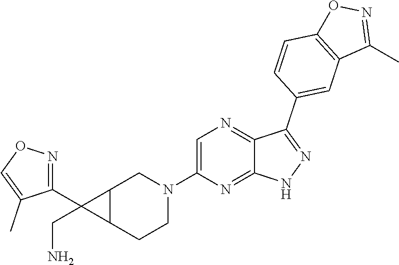 [SMILES: Cc1conc1C1(CN)C2CCN(c3cnc4c(-c5ccc6onc(C)c6c5)n[nH]c4n3)CC21] | (3-(3-(3-methylbenzo[d]isoxazol-5-yl)-1H- pyrazolo[3,4-b]pyrazin-6-yl)-7-(4-methylisoxazol-3- yl)-3-azabicyclo[4.1.0]heptan-7-yl)methanamine | 456.5 |
| 53 | 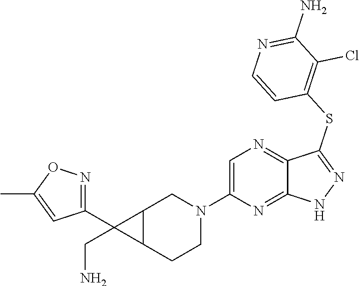 [SMILES: Cc1cc(C2(CN)C3CCN(c4cnc5c(Sc6ccnc(N)c6Cl)n[nH]c5n4)CC32)no1] | 4-((6-(7-(aminomethyl)-7-(5-methylisoxazol-3-yl)-3- azabicyclo[4.1.0]heptan-3-yl)-1H-pyrazolo[3,4- b]pyrazin-3-yl)thio)-3-chloropyridin-2-amine | 483.98 |
| 54 | 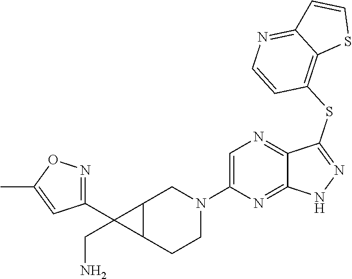 [SMILES: Cc1cc(C2(CN)C3CCN(c4cnc5c(Sc6ccnc7ccsc67)n[nH]c5n4)CC32)no1] | (7-(5-methylisoxazol-3-yl)-3-(3-(thieno[3,2- b]pyridin-7-ylthio)-1H-pyrazolo[3,4-b]pyrazin-6-yl)- 3-azabicyclo[4.1.0]heptan-7-yl)methanamine | 490.61 |
| 55 | 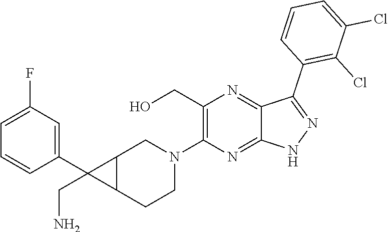 [SMILES: NCC1(c2cccc(F)c2)C2CCN(c3nc4[nH]nc(-c5cccc(Cl)c5Cl)c4nc3CO)CC21] | (6-(7-(aminomethyl)-7-(3-fluorophenyl)-3- azabicyclo[4.1.0]heptan-3-yl)-3-(2,3- dichlorophenyl)-1H-pyrazolo[3,4-b]pyrazin-5- yl)methanol | 513.39 |
| 56 | 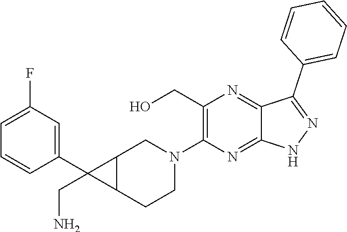 [SMILES: NCC1(c2cccc(F)c2)C2CCN(c3nc4[nH]nc(-c5ccccc5)c4nc3CO)CC21] | (6-(7-(aminomethyl)-7-(3-fluorophenyl)-3- azabicyclo[4.1.0]heptan-3-yl)-3-phenyl-1H- pyrazolo[3,4-b]pyrazin-5-yl)methanol | 444.5 |
| 57 | 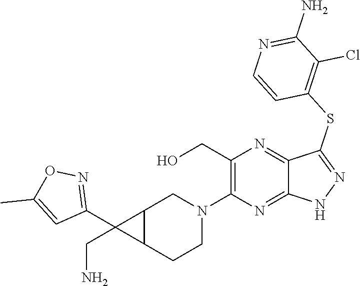 [SMILES: Cc1cc(C2(CN)C3CCN(c4nc5[nH]nc(Sc6ccnc(N)c6Cl)c5nc4CO)CC32)no1] | (3-((2-amino-3-chloropyridin-4-yl)thio)-6-(7- (aminomethyl)-7-(5-methylisoxazol-3-yl)-3- azabicyclo[4.1.0]heptan-3-yl)-1H-pyrazolo[3,4- b]pyrazin-5-yl)methanol | 514 |
| 58 | 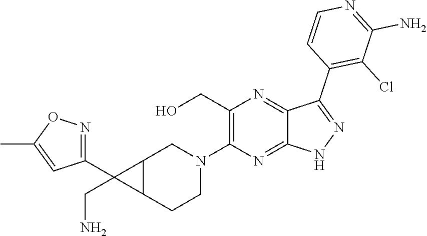 [SMILES: Cc1cc(C2(CN)C3CCN(c4nc5[nH]nc(-c6ccnc(N)c6Cl)c5nc4CO)CC32)no1] | (3-(2-amino-3-chloropyridin-4-yl)-6-(7- (aminomethyl)-7-(5-methylisoxazol-3-yl)-3- azabicyclo[4.1.0]heptan-3-yl)-1H-pyrazolo[3,4- b]pyrazin-5-yl)methanol | 481.94 |
| 59 | 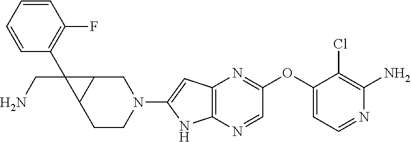 [SMILES: NCC1(c2ccccc2F)C2CCN(c3cc4nc(Oc5ccnc(N)c5Cl)cnc4[nH]3)CC21] | 4-((6-(7-(aminomethyl)-7-(2-fluorophenyl)-3- azabicyclo[4.1.0]heptan-3-yl)-5H-pyrrolo[2,3- b]pyrazin-2-yl)thio)-3-chloropyridin-2-amine | 496 |
| 60 | 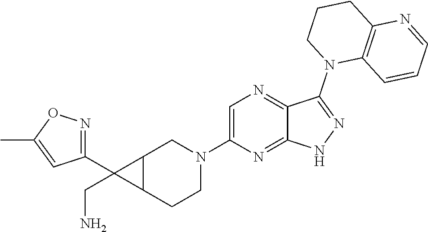 [SMILES: Cc1cc(C2(CN)C3CCN(c4cnc5c(N6CCCc7ncccc76)n[nH]c5n4)CC32)no1] | (3-(3-(3,4-dihydro-1,5-naphthyridin-1(2H)-yl)-1H- pyrazolo[3,4-b]pyrazin-6-yl)-7-(5-methylisoxazol-3- yl)-3-azabicyclo[4.1.0]heptan-7-yl)methanamine | 457.53 |
| 61 | 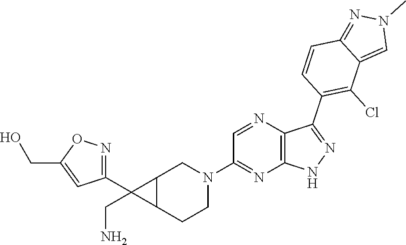 [SMILES: Cn1cc2c(Cl)c(-c3n[nH]c4nc(N5CCC6C(C5)C6(CN)c5cc(CO)on5)cnc34)ccc2n1] | (3-(7-(aminomethyl)-3-(3-(4-chloro-2-methyl-2H- indazol-5-yl)-1H-pyrazolo[3,4-b]pyrazin-6-yl)-3- azabicyclo[4.1.0]heptan-7-yl)isoxazol-5-yl)methanol | 505.96 |
| 62 | 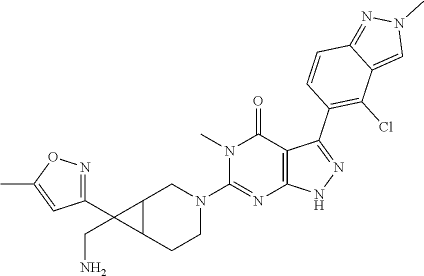 | 6-(7-(aminomethyl)-7-(5-methylisoxazol-3-yl)-3- azabicyclo[4.1.0]heptan-3-yl)-3-(4-chloro-2-methyl- 2H-indazol-5-yl)-5-methyl-1,5-dihydro-4H- pyrazolo[3,4-d]pyrimidin-4-one | 519.99 |
| 63 | 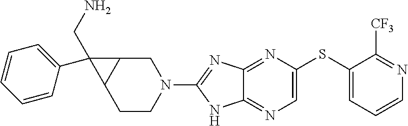 [SMILES: NCC1(c2ccccc2)C2CCN(c3nc4nc(Sc5cccnc5C(F)(F)F)cnc4[nH]3)CC21] | (7-phenyl-3-(5-((2-(trifluoromethyl)pyridin-3- yl)thio)-1H-imidazo[4,5-b]pyrazin-2-yl)-3- azabicyclo[4.1.0]heptan-7-yl)methanamine | 497.54 |
| 64 | 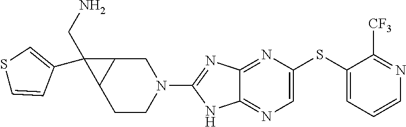 [SMILES: NCC1(c2ccsc2)C2CCN(c3nc4nc(Sc5cccnc5C(F)(F)F)cnc4[nH]3)CC21] | (7-(thiophen-3-yl)-3-(5-((2-(trifluoromethyl)pyridin- 3-yl)thio)-1H-imidazo[4,5-b]pyrazin-2-yl)-3- azabicyclo[4.1.0]heptan-7-yl)methanamine | 503.57 |
| 65 | 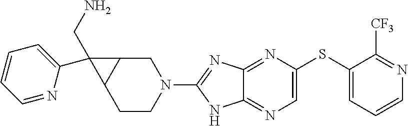 [SMILES: NCC1(c2ccccn2)C2CCN(c3nc4nc(Sc5cccnc5C(F)(F)F)cnc4[nH]3)CC21] | (7-(pyridin-2-yl)-3-(5-((2-(trifluoromethyl)pyridin-3- yl)thio)-1H-imidazo[4,5-b]pyrazin-2-yl)-3- azabicyclo[4.1.0]heptan-7-yl)methanamine | 498.53 |
| 66 | 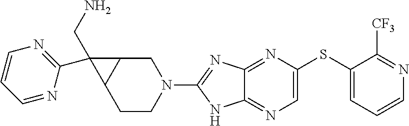 [SMILES: NCC1(c2ncccn2)C2CCN(c3nc4nc(Sc5cccnc5C(F)(F)F)cnc4[nH]3)CC21] | (7-(pyrimidin-2-yl)-3-(5-((2-(trifluoromethyl)pyridin- 3-yl)thio)-1H-imidazo[4,5-b]pyrazin-2-yl)-3- azabicyclo[4.1.0]heptan-7-yl)methanamine | 499.52 |
| 67 | 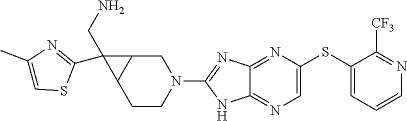 [SMILES: Cc1csc(C2(CN)C3CCN(c4nc5nc(Sc6cccnc6C(F)(F)F)cnc5[nH]4)CC32)n1] | (7-(4-methylthiazol-2-yl)-3-(5-((2- (trifluoromethyl)pyridin-3-yl)thio)-1H-imidazo[4,5- b]pyrazin-2-yl)-3-azabicyclo[4.1.0]heptan-7- yl)methanamine | 518.58 |
| 68 | 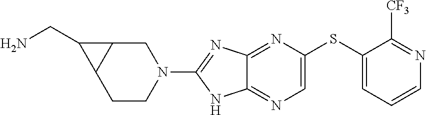 [SMILES: NCC1C2CCN(c3nc4nc(Sc5cccnc5C(F)(F)F)cnc4[nH]3)CC12] | (3-(5-((2-(trifluoromethyl)pyridin-3-yl)thio)-1H- imidazo[4,5-b]pyrazin-2-yl)-3- azabicyclo[4.1.0]heptan-7-yl)methanamine | 421.44 |
| 69 | 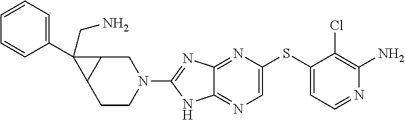 [SMILES: NCC1(c2ccccc2)C2CCN(c3nc4nc(Sc5ccnc(N)c5Cl)cnc4[nH]3)CC21] | 4-((2-(7-(aminomethyl)-7-phenyl-3- azabicyclo[4.1.0]heptan-3-yl)-1H-imidazo[4,5- b]pyrazin-5-yl)thio)-3-chloropyridin-2-amine | 479 |
| 70 | 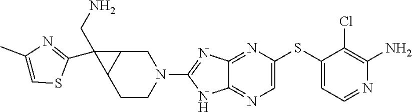 [SMILES: Cc1csc(C2(CN)C3CCN(c4nc5nc(Sc6ccnc(N)c6Cl)cnc5[nH]4)CC32)n1] | 4-((2-(7-(aminomethyl)-7-(4-methylthiazol-2-yl)-3- azabicyclo[4.1.0]heptan-3-yl)-1H-imidazo[4,5- b]pyrazin-5-yl)thio)-3-chloropyridin-2-amine | 500.05 |
| 71 | 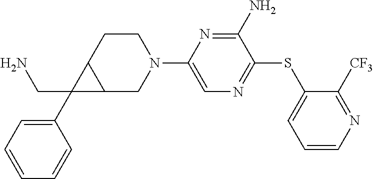 [SMILES: NCC1(c2ccccc2)C2CCN(c3cnc(Sc4cccnc4C(F)(F)F)c(N)n3)CC21] | 6-(7-(aminomethyl)-7-phenyl-3- azabicyclo[4.1.0]heptan-3-yl)-3-((2- (trifluoromethyl)pyridin-3-yl)thio)pyrazin-2-amine | 472.53 |
| 72 | 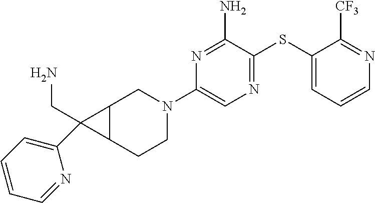 [SMILES: NCC1(c2ccccn2)C2CCN(c3cnc(Sc4cccnc4C(F)(F)F)c(N)n3)CC21] | 6-(7-(aminomethyl)-7-(pyridin-2-yl)-3- azabicyclo[4.1.0]heptan-3-yl)-3-((2- (trifluoromethyl)pyridin-3-yl)thio)pyrazin-2-amine | 473.52 |
| 73 | 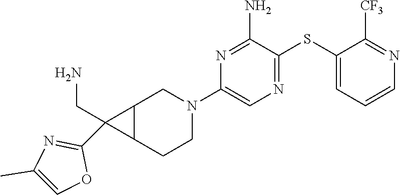 [SMILES: Cc1coc(C2(CN)C3CCN(c4cnc(Sc5cccnc5C(F)(F)F)c(N)n4)CC32)n1] | 6-(7-(aminomethyl)-7-(4-methylthiazol-2-yl)-3- azabicyclo[4.1.0]heptan-3-yl)-3-((2- (trifluoromethyl)pyridin-3-yl)thio)pyrazin-2-amine | 493.57 |
| 74 | 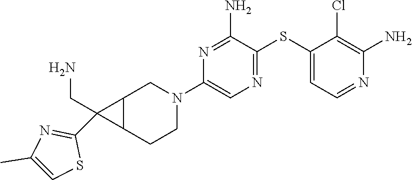 [SMILES: Cc1csc(C2(CN)C3CCN(c4cnc(Sc5ccnc(N)c5Cl)c(N)n4)CC32)n1] | 3-((2-amino-3-chloropyridin-4-yl)thio)-6-(7- (aminomethyl)-7-(4-methylthiazol-2-yl)-3- azabicyclo[4.1.0]heptan-3-yl)pyrazin-2-amine | 475.04 |
| 75 | 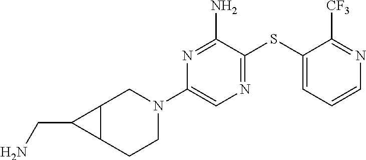 [SMILES: NCC1C2CCN(c3cnc(Sc4cccnc4C(F)(F)F)c(N)n3)CC12] | 6-(7-(aminomethyl)-3-azabicyclo[4.1.0]heptan-3-yl)- 3-((2-(trifluoromethyl)pyridin-3-yl)thio)pyrazin-2- amine | 396.43 |
| 76 | 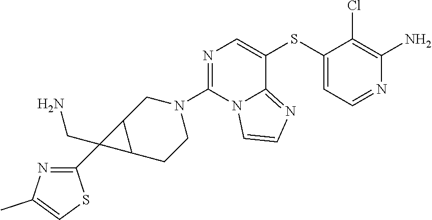 [SMILES: Cc1csc(C2(CN)C3CCN(c4ncc(Sc5ccnc(N)c5Cl)c5nccn45)CC32)n1] | 4-((5-(7-(aminomethyl)-7-(4-methylthiazol-2-yl)-3- azabicyclo[4.1.0]heptan-3-yl)imidazo[1,2- c]pyrimidin-8-yl)thio)-3-chloropyridin-2-amine | 499.06 |
| 77 | 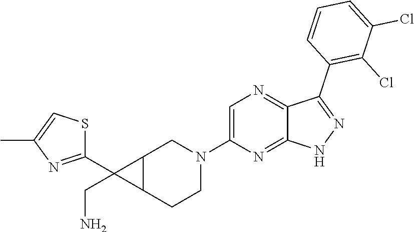 [SMILES: Cc1csc(C2(CN)C3CCN(c4cnc5c(-c6cccc(Cl)c6Cl)n[nH]c5n4)CC32)n1] | (3-(3-(2,3-dichlorophenyl)-1H-pyrazolo[3,4- b]pyrazin-6-yl)-7-(4-methylthiazol-2-yl)-3- azabicyclo[4.1.0]heptan-7-yl)methanamine | 486.42 |
| 78 | 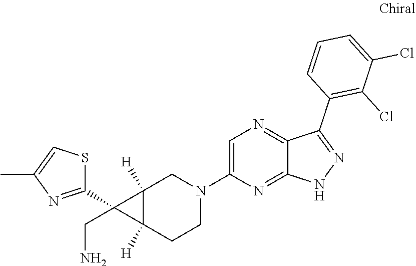 [SMILES: Cc1csc([C@@]2(CN)[C@@H]3CCN(c4cnc5c(-c6cccc(Cl)c6Cl)n[nH]c5n4)C[C@@H]32)n1] | ((1S,6R,7S)-3-(3-(2,3-dichlorophenyl)-1H- pyrazolo[3,4-b]pyrazin-6-yl)-7-(4-methylthiazol-2- yl)-3-azabicyclo[4.1.0]heptan-7-yl)methanamine | 486.42 |
| 79 | 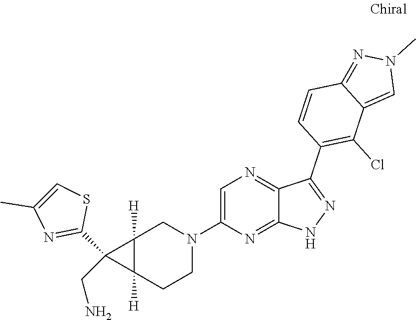 | ((1S,6R,7S)-3-(3-(4-chloro-2-methyl-2H-indazol-5- yl)-1H-pyrazolo[3,4-b]pyrazin-6-yl)-7-(4- methylthiazol-2-yl)-3-azabicyclo[4.1.0]heptan-7- yl)methanamine | 506.03 |
| 80 | 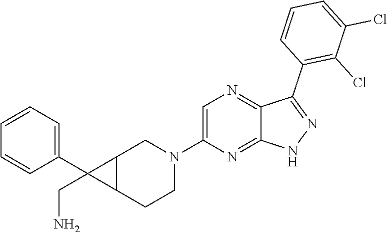 [SMILES: NCC1(c2ccccc2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-phenyl-3-azabicyclo[4.1.0]heptan- 7-yl)methanamine | 465.38 |
| 81 | 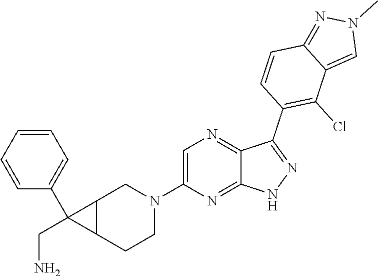 [SMILES: Cn1cc2c(Cl)c(-c3n[nH]c4nc(N5CCC6C(C5)C6(CN)c5ccccc5)cnc34)ccc2n1] | (3-(3-(4-chloro-2-methyl-2H-indazol-5-yl)-1H- pyrazolo[3,4-b]pyrazin-6-yl)-7-phenyl-3- azabicyclo[4.1.0]heptan-7-yl)methanamine | 484.98 |
| 82 | 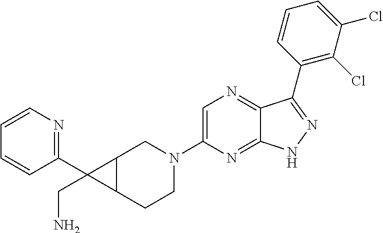 [SMILES: NCC1(c2ccccn2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(pyridin-2-yl)-3- azabicyclo[4.1.0]heptan-7-yl)methanamine | 466.37 |
| 83 | 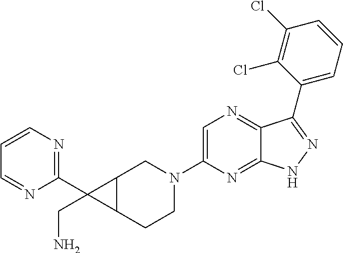 [SMILES: NCC1(c2ncccn2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(pyrimidin-2-yl)-3- azabicyclo[4.1.0]heptan-7-yl)methanamine | 467.35 |
| 84 | 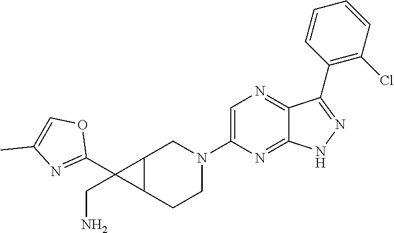 [SMILES: Cc1coc(C2(CN)C3CCN(c4cnc5c(-c6ccccc6Cl)n[nH]c5n4)CC32)n1] | (3-(3-(2-chlorophenyl)-1H-pyrazolo[3,4-b]pyrazin-6- yl)-7-(4-methylthiazol-2-yl)-3- azabicyclo[4.1.0]heptan-7-yl)methanamine | 451.98 |
| 85 | 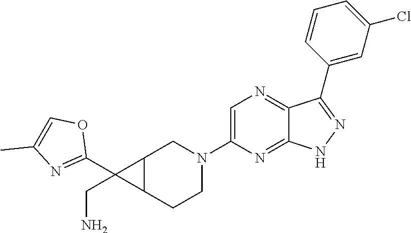 [SMILES: Cc1coc(C2(CN)C3CCN(c4cnc5c(-c6cccc(Cl)c6)n[nH]c5n4)CC32)n1] | (3-(3-(3-chlorophenyl)-1H-pyrazolo[3,4-b]pyrazin-6- yl)-7-(4-methylthiazol-2-yl)-3- azabicyclo[4.1.0]heptan-7-yl)methanamine | 451.98 |
| 86 | 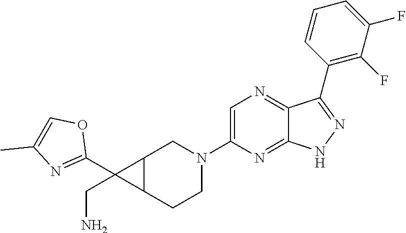 [SMILES: Cc1coc(C2(CN)C3CCN(c4cnc5c(-c6cccc(F)c6F)n[nH]c5n4)CC32)n1] | (3-(3-(2,3-difluorophenyl)-1H-pyrazolo[3,4- b]pyrazin-6-yl)-7-(4-methylthiazol-2-yl)-3- azabicyclo[4.1.0]heptan-7-yl)methanamine | 453.51 |
| 87 | 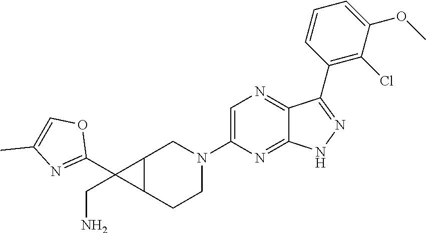 | (3-(3-(2-chloro-3-methoxyphenyl)-1H-pyrazolo[3,4- b]pyrazin-6-yl)-7-(4-methylthiazol-2-yl)-3- azabicyclo[4.1.0]heptan-7-yl)methanamine | 482 |
| 88 | 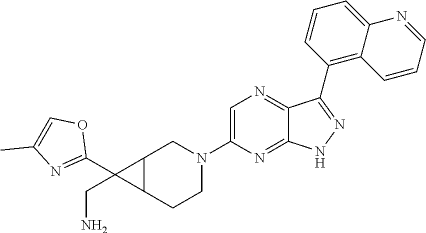 [SMILES: Cc1coc(C2(CN)C3CCN(c4cnc5c(-c6cccc7ncccc67)n[nH]c5n4)CC32)n1] | (7-(4-methylthiazol-2-yl)-3-(3-(quinolin-5-yl)-1H- pyrazolo[3,4-b]pyrazin-6-yl)-3- azabicyclo[4.1.0]heptan-7-yl)methanamine | 468.58 |
| 89 | 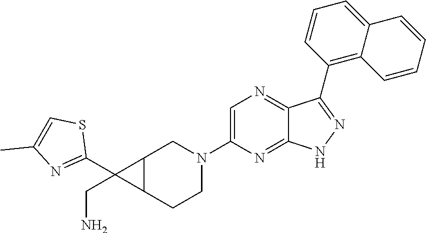 [SMILES: Cc1csc(C2(CN)C3CCN(c4cnc5c(-c6cccc7ccccc67)n[nH]c5n4)CC32)n1] | (7-(4-methylthiazol-2-yl)-3-(3-(naphthalen-1-yl)-1H- pyrazolo[3,4-b]pyrazin-6-yl)-3- azabicyclo[4.1.0]heptan-7-yl)methanamine | 467.59 |
| 90 | 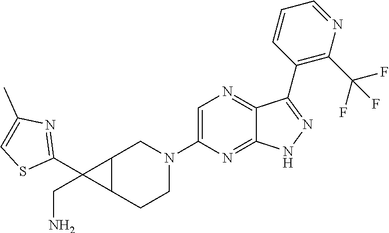 [SMILES: Cc1csc(C2(CN)C3CCN(c4cnc5c(-c6cccnc6C(F)(F)F)n[nH]c5n4)CC32)n1] | (7-(4-methylthiazol-2-yl)-3-(3-(2- (trifluoromethyl)pyridin-3-yl)-1H-pyrazolo[3,4- b]pyrazin-6-yl)-3-azabicyclo[4.1.0]heptan-7- yl)methanamine | 486.52 |
| 91 | 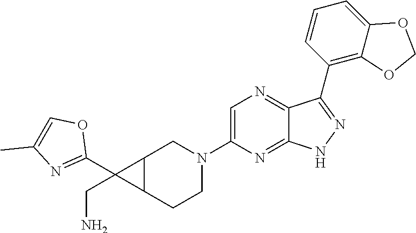 [SMILES: Cc1coc(C2(CN)C3CCN(c4cnc5c(-c6cccc7c6OCO7)n[nH]c5n4)CC32)n1] | (3-(3-(benzo[d][1,3]dioxol-4-yl)-1H-pyrazolo[3,4- b]pyrazin-6-yl)-7-(4-methylthiazol-2-yl)-3- azabicyclo[4.1.0]heptan-7-yl)methanamine | 461.54 |
| 92 | 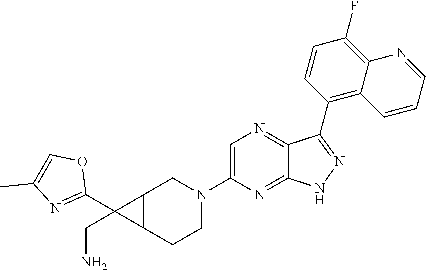 [SMILES: Cc1coc(C2(CN)C3CCN(c4cnc5c(-c6ccc(F)c7ncccc67)n[nH]c5n4)CC32)n1] | (3-(3-(8-fluoroquinolin-5-yl)-1H-pyrazolo[3,4- b]pyrazin-6-yl)-7-(4-methylthiazol-2-yl)-3- azabicyclo[4.1.0]heptan-7-yl)methanamine | 486.57 |
| 93 | 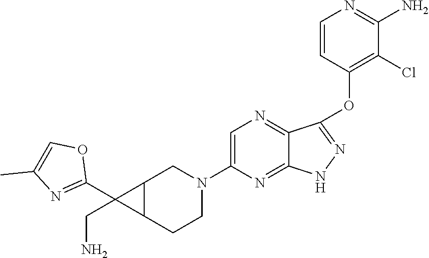 [SMILES: Cc1coc(C2(CN)C3CCN(c4cnc5c(Oc6ccnc(N)c6Cl)n[nH]c5n4)CC32)n1] | 4-((6-(7-(aminomethyl)-7-(4-methylthiazol-2-yl)-3- azabicyclo[4.1.0]heptan-3-yl)-1H-pyrazolo[3,4- b]pyrazin-3-yl)thio)-3-chloropyridin-2-amine | 500.05 |
| 94 | 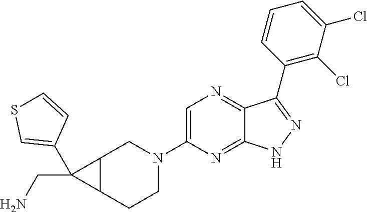 [SMILES: NCC1(c2ccsc2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(thiophen-3-yl)-3- azabicyclo[4.1.0]heptan-7-yl)methanamine | 471.41 |
| 95 | 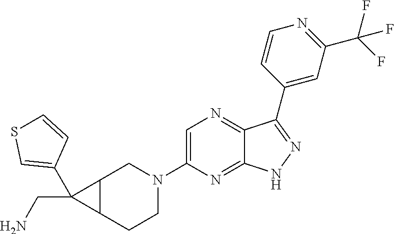 [SMILES: NCC1(c2ccsc2)C2CCN(c3cnc4c(-c5ccnc(C(F)(F)F)c5)n[nH]c4n3)CC21] | (7-(thiophen-3-yl)-3-(3-(2-(trifluoromethyl)pyridin-4- yl)-1H-pyrazolo[3,4-b]pyrazin-6-yl)-3- azabicyclo[4.1.0]heptan-7-yl)methanamine | 471.5 |
| 96 | 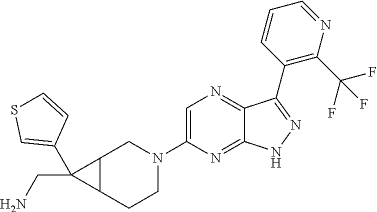 [SMILES: NCC1(c2ccsc2)C2CCN(c3cnc4c(-c5cccnc5C(F)(F)F)n[nH]c4n3)CC21] | (7-(thiophen-3-yl)-3-(3-(2-(trifluoromethyl)pyridin-3- yl)-1H-pyrazolo[3,4-b]pyrazin-6-yl)-3- azabicyclo[4.1.0]heptan-7-yl)methanamine | 471.5 |
| 97 | 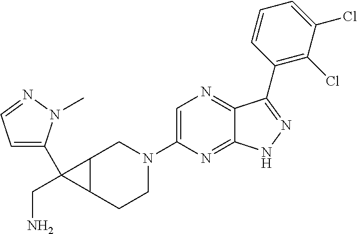 [SMILES: Cn1nccc1C1(CN)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(1-methyl-1H-pyrazol-5-yl)-3- azabicyclo[4.1.0]heptan-7-yl)methanamine | 469.37 |
| 98 | 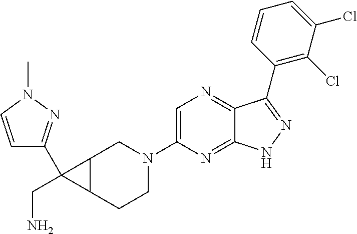 [SMILES: Cn1ccc(C2(CN)C3CCN(c4cnc5c(-c6cccc(Cl)c6Cl)n[nH]c5n4)CC32)n1] | (3-(3-(2,3-dichlorophenyl)-1H-pyrazolo[3,4- b]pyrazin-6-yl)-7-(1-methyl-1H-pyrazol-3-yl)-3- azabicyclo[4.1.0]heptan-7-yl)methanamine | 469.37 |
| 99 | 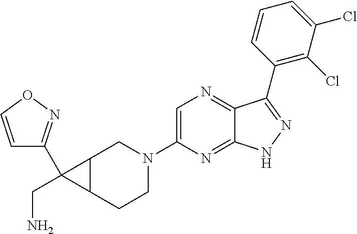 [SMILES: NCC1(c2ccon2)C2CCN(c3cnc4c(-c5cccc(Cl)c5Cl)n[nH]c4n3)CC21] | (3-(3-(2,3-dichlorophenyl)-1H-pyrazolo[3,4- b]pyrazin-6-yl)-7-(isoxazol-3-yl)-3- azabicyclo[4.1.0]heptan-7-yl)methanamine | 456.33 |
| 100 | 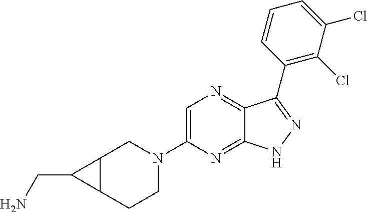 [SMILES: NCC1C2CCN(c3cnc4c(-c5cccc(Cl)c5Cl)n[nH]c4n3)CC12] | (3-(3-(2,3-dichlorophenyl)-1H-pyrazolo[3,4- b]pyrazin-6-yl)-3-azabicyclo[4.1.0]heptan-7- yl)methanamine | 389.28 |
| 101 | 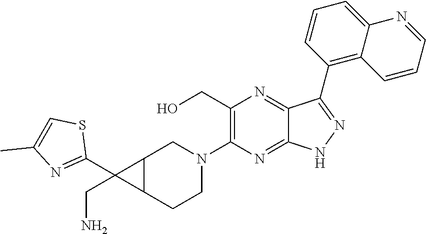 [SMILES: Cc1csc(C2(CN)C3CCN(c4nc5[nH]nc(-c6cccc7ncccc67)c5nc4CO)CC32)n1] | (6-(7-(aminomethyl)-7-(4-methylthiazol-2-yl)-3- azabicyclo[4.1.0]heptan-3-yl)-3-(quinolin-5-yl)-1H- pyrazolo[3,4-b]pyrazin-5-yl)methanol | 498.6 |
| 102 | 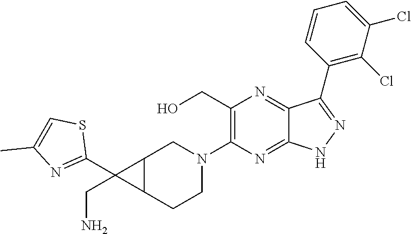 [SMILES: Cc1csc(C2(CN)C3CCN(c4nc5[nH]nc(-c6cccc(Cl)c6Cl)c5nc4CO)CC32)n1] | (6-(7-(aminomethyl)-7-(4-methylthiazol-2-yl)-3- azabicyclo[4.1.0]heptan-3-yl)-3-(2,3- dichlorophenyl)-1H-pyrazolo[3,4-b]pyrazin-5- yl)methanol | 516.45 |
| 103 | 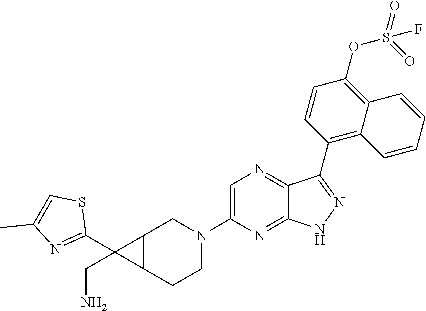 [SMILES: Cc1csc(C2(CN)C3CCN(c4cnc5c(-c6ccc(OS(=O)(=O)F)c7ccccc67)n[nH]c5n4)CC32)n1] | 4-(6-(7-(aminomethyl)-7-(4-methylthiazol-2-yl)-3- azabicyclo[4.1.0]heptan-3-yl)-1H-pyrazolo[3,4- b]pyrazin-3-yl)naphthalen-1-yl sulfurofluoridate | 565.65 |
| 104 | 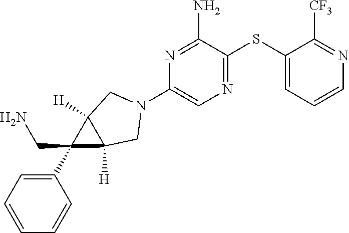 [SMILES: NC[C@]1(c2ccccc2)[C@@H]2CN(c3cnc(Sc4cccnc4C(F)(F)F)c(N)n3)C[C@@H]21] | 6-((1R,5S,6s)-6-(aminomethyl)-6-phenyl-3- azabicyclo[3.1.0]hexan-3-yl)-3-((2- (trifluoromethyl)pyridin-3-yl)thio)pyrazin-2-amine | 458.5 |
| 105 | 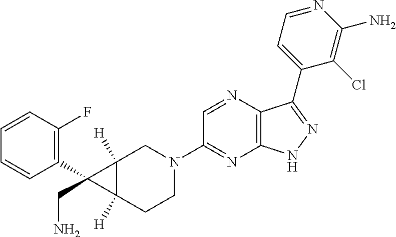 [SMILES: NC[C@]1(c2ccccc2F)[C@@H]2CCN(c3cnc4c(-c5ccnc(N)c5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloropyridin-2-amine | 465.93 |
| 106 | 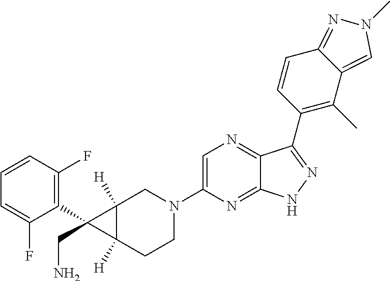 [SMILES: Cc1c(-c2n[nH]c3nc(N4CC[C@@H]5[C@H](C4)[C@@]5(CN)c4c(F)cccc4F)cnc23)ccc2nn(C)cc12] | ((1S,6R,7R)-7-(2,6-difluorophenyl)-3-(3-(2,4- dimethyl-2H-indazol-5-yl)-1H-pyrazolo[3,4- b]pyrazin-6-yl)-3-azabicyclo[4.1.0]heptan-7- yl)methanamine | 500.55 |
| 107 | 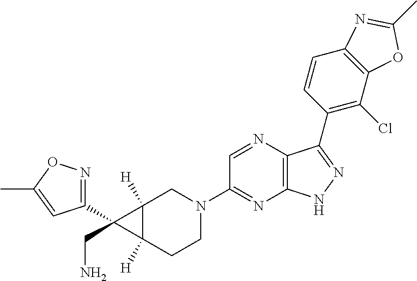 | ((1S,6R,7S)-3-(3-(7-chloro-2-methylbenzo[d]thiazol- 6-yl)-1H-pyrazolo[3,4-b]pyrazin-6-yl)-7-(5- methylisoxazol-3-yl)-3-azabicyclo[4.1.0]heptan-7- yl)methanamine | 507.01 |
| 108 | 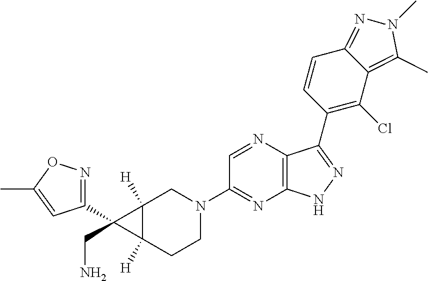 | ((1S,6R,7S)-3-(3-(4-chloro-2,3-dimethyl-2H-indazol- 5-yl)-1H-pyrazolo[3,4-b]pyrazin-6-yl)-7-(5- methylisoxazol-3-yl)-3-azabicyclo[4.1.0]heptan-7- yl)methanamine | 503.99 |
| 109 | 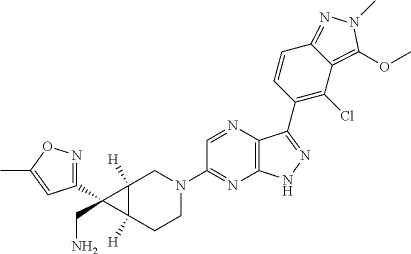 | ((1S,6R,7S)-3-(3-(4-chloro-3-methoxy-2-methyl-2H- indazol-5-yl)-1H-pyrazolo[3,4-b]pyrazin-6-yl)-7-(5- methylisoxazol-3-yl)-3-azabicyclo[4.1.0]heptan-7- yl)methanamine | 519.99 |
| 110 | 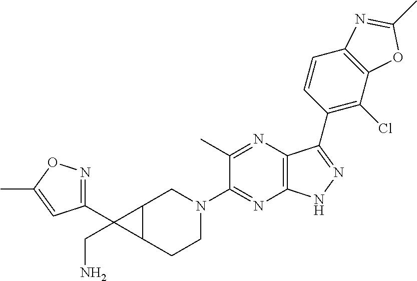 | (3-(3-(7-chloro-2-methylbenzo[d]thiazol-6-yl)-5- methyl-1H-pyrazolo[3,4-b]pyrazin-6-yl)-7-(5- methylisoxazol-3-yl)-3-azabicyclo[4.1.0]heptan-7- yl)methanamine | 521.04 |
| 111 | 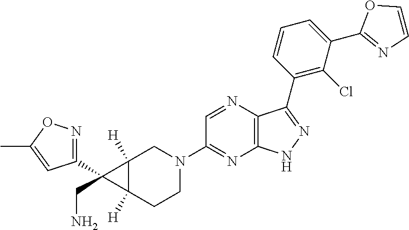 [SMILES: Cc1cc([C@@]2(CN)[C@@H]3CCN(c4cnc5c(-c6cccc(-c7ncco7)c6Cl)n[nH]c5n4)C[C@@H]32)no1] | ((1S,6R,7S)-3-(3-(2-chloro-3-(oxazol-2-yl)phenyl)- 1H-pyrazolo[3,4-b]pyrazin-6-yl)-7-(5- methylisoxazol-3-yl)-3-azabicyclo[4.1.0]heptan-7- yl)methanamine | 502.96 |
| 112 | 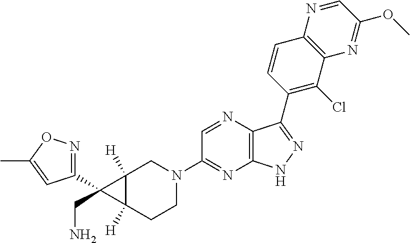 | ((1S,6R,7S)-3-(3-(5-chloro-3-methoxyquinoxalin-6- yl)-1H-pyrazolo[3,4-b]pyrazin-6-yl)-7-(5- methylisoxazol-3-yl)-3-azabicyclo[4.1.0]heptan-7- yl)methanamine | 517.97 |
| 113 | 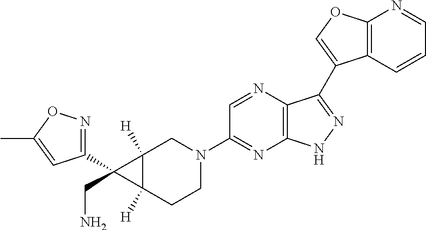 [SMILES: Cc1cc([C@@]2(CN)[C@@H]3CCN(c4cnc5c(-c6coc7ncccc67)n[nH]c5n4)C[C@@H]32)no1] | ((1S,6R,7S)-7-(5-methylisoxazol-3-yl)-3-(3- (thieno[2,3-b]pyridin-3-yl)-1H-pyrazolo[3,4- b]pyrazin-6-yl)-3-azabicyclo[4.1.0]heptan-7- yl)methanamine | 458.54 |
| 114 | 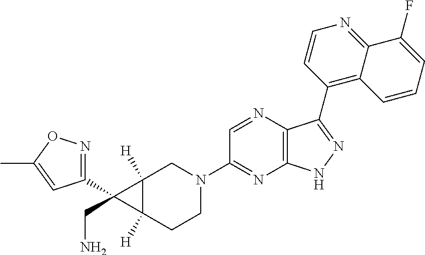 [SMILES: Cc1cc([C@@]2(CN)[C@@H]3CCN(c4cnc5c(-c6ccnc7c(F)cccc67)n[nH]c5n4)C[C@@H]32)no1] | ((1S,6R,7S)-3-(3-(8-fluoroquinolin-4-yl)-1H- pyrazolo[3,4-b]pyrazin-6-yl)-7-(5-methylisoxazol-3- yl)-3-azabicyclo[4.1.0]heptan-7-yl)methanamine | 470.5 |
| 115 | 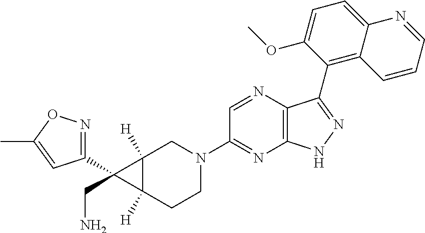 [SMILES: COc1ccc2ncccc2c1-c1n[nH]c2nc(N3CC[C@@H]4[C@H](C3)[C@@]4(CN)c3cc(C)on3)cnc12] | ((1S,6R,7S)-3-(3-(6-methoxyquinolin-5-yl)-1H- pyrazolo[3,4-b]pyrazin-6-yl)-7-(5-methylisoxazol-3- yl)-3-azabicyclo[4.1.0]heptan-7-yl)methanamine | 482.54 |
| 116 | 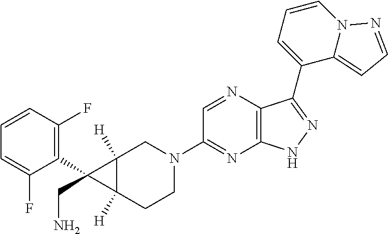 [SMILES: NC[C@]1(c2c(F)cccc2F)[C@@H]2CCN(c3cnc4c(-c5cccn6nccc56)n[nH]c4n3)C[C@@H]21] | ((1S,6R,7R)-7-(2,6-difluorophenyl)-3-(3- (pyrazolo[1,5-a]pyridin-4-yl)-1H-pyrazolo[3,4- b]pyrazin-6-yl)-3-azabicyclo[4.1.0]heptan-7- yl)methanamine | 472.49 |
| 117 | 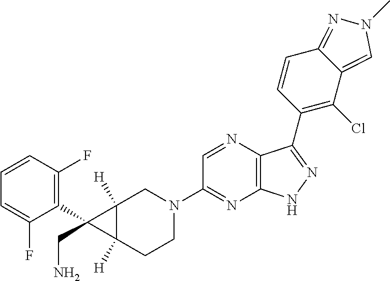 [SMILES: Cn1cc2c(Cl)c(-c3n[nH]c4nc(N5CC[C@@H]6[C@H](C5)[C@@]6(CN)c5c(F)cccc5F)cnc34)ccc2n1] | ((1S,6R,7R)-3-(3-(4-chloro-2-methyl-2H-indazol-5- yl)-1H-pyrazolo[3,4-b]pyrazin-6-yl)-7-(2,6- difluorophenyl)-3-azabicyclo[4.1.0]heptan-7- yl)methanamine | 520.97 |
| 118 | 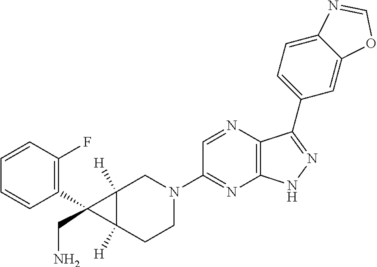 [SMILES: NC[C@]1(c2ccccc2F)[C@@H]2CCN(c3cnc4c(-c5ccc6ncoc6c5)n[nH]c4n3)C[C@@H]21] | ((1S,6R,7R)-3-(3-(benzo[d]thiazol-6-yl)-1H- pyrazolo[3,4-b]pyrazin-6-yl)-7-(2-fluorophenyl)-3- azabicyclo[4.1.0]heptan-7-yl)methanamine | 471.55 |
| 119 | 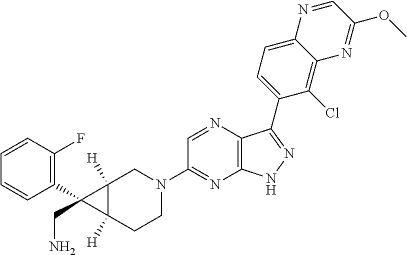 [SMILES: COc1cnc2ccc(-c3n[nH]c4nc(N5CC[C@@H]6[C@H](C5)[C@@]6(CN)c5ccccc5F)cnc34)c(Cl)c2n1] | ((1S,6R,7R)-3-(3-(5-chloro-3-methoxyquinoxalin-6- yl)-1H-pyrazolo[3,4-b]pyrazin-6-yl)-7-(2- fluorophenyl)-3-azabicyclo[4.1.0]heptan-7- yl)methanamine | 530.98 |
| 120 | 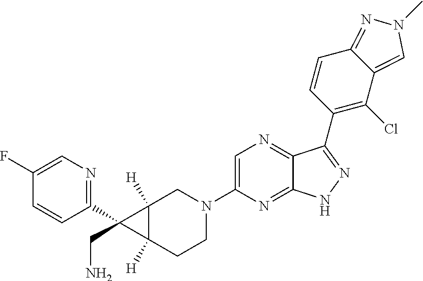 [SMILES: Cn1cc2c(Cl)c(-c3n[nH]c4nc(N5CC[C@@H]6[C@H](C5)[C@@]6(CN)c5ccc(F)cn5)cnc34)ccc2n1] | ((1S,6R,7S)-3-(3-(4-chloro-2-methyl-2H-indazol-5- yl)-1H-pyrazolo[3,4-b]pyrazin-6-yl)-7-(5- fluoropyridin-2-yl)-3-azabicyclo[4.1.0]heptan-7- yl)methanamine | 503.96 |
| 121 | 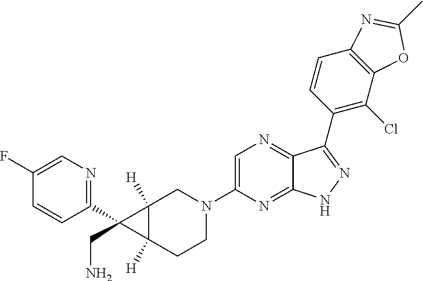 [SMILES: Cc1nc2ccc(-c3n[nH]c4nc(N5CC[C@@H]6[C@H](C5)[C@@]6(CN)c5ccc(F)cn5)cnc34)c(Cl)c2o1] | ((1S,6R,7S)-3-(3-(7-chloro-2-methylbenzo[d]thiazol- 6-yl)-1H-pyrazolo[3,4-b]pyrazin-6-yl)-7-(5- fluoropyridin-2-yl)-3-azabicyclo[4.1.0]heptan-7- yl)methanamine | 521.01 |
| 122 | 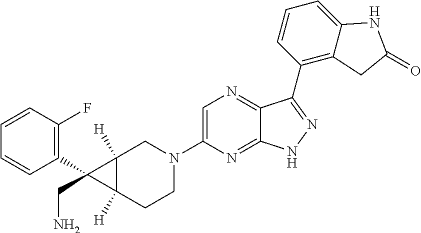 [SMILES: NC[C@]1(c2ccccc2F)[C@@H]2CCN(c3cnc4c(-c5cccc6c5CC(=O)N6)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)indolin-2-one | 469.51 |
| 123 | 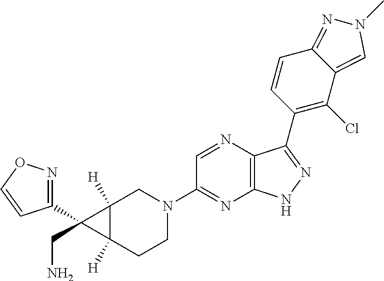 [SMILES: Cn1cc2c(Cl)c(-c3n[nH]c4nc(N5CC[C@@H]6[C@H](C5)[C@@]6(CN)c5ccon5)cnc34)ccc2n1] | ((1S,6R,7S)-3-(3-(4-chloro-2-methyl-2H-indazol-5- yl)-1H-pyrazolo[3,4-b]pyrazin-6-yl)-7-(isoxazol-3- yl)-3-azabicyclo[4.1.0]heptan-7-yl)methanamine | 475.93 |
| 124 | 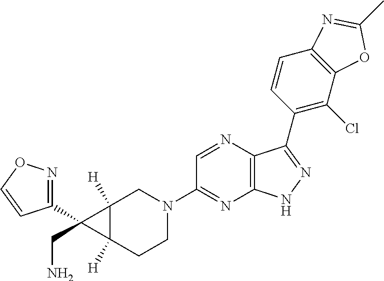 [SMILES: Cc1nc2ccc(-c3n[nH]c4nc(N5CC[C@@H]6[C@H](C5)[C@@]6(CN)c5ccon5)cnc34)c(Cl)c2o1] | ((1S,6R,7S)-3-(3-(7-chloro-2-methylbenzo[d]thiazol- 6-yl)-1H-pyrazolo[3,4-b]pyrazin-6-yl)-7-(isoxazol-3- yl)-3-azabicyclo[4.1.0]heptan-7-yl)methanamine | 492.99 |
| 125 | 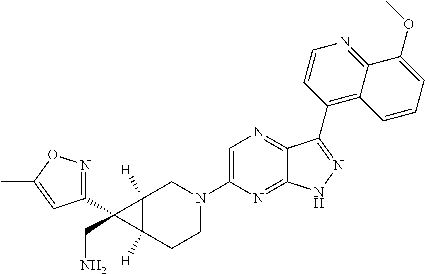 [SMILES: COc1cccc2c(-c3n[nH]c4nc(N5CC[C@@H]6[C@H](C5)[C@@]6(CN)c5cc(C)on5)cnc34)ccnc12] | ((1S,6R,7S)-3-(3-(8-methoxyquinolin-4-yl)-1H- pyrazolo[3,4-b]pyrazin-6-yl)-7-(5-methylisoxazol-3- yl)-3-azabicyclo[4.1.0]heptan-7-yl)methanamine | 482.54 |
| 126 | 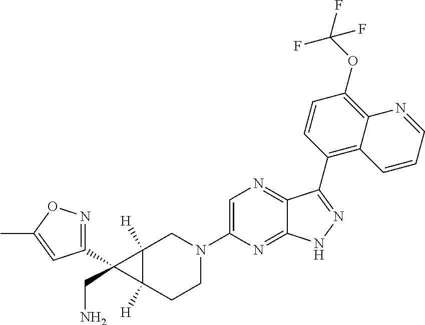 [SMILES: Cc1cc([C@@]2(CN)[C@@H]3CCN(c4cnc5c(-c6ccc(OC(F)(F)F)c7ncccc67)n[nH]c5n4)C[C@@H]32)no1] | ((1S,6R,7S)-7-(5-methylisoxazol-3-yl)-3-(3-(8- (trifluoromethoxy)quinolin-5-yl)-1H-pyrazolo[3,4- b]pyrazin-6-yl)-3-azabicyclo[4.1.0]heptan-7- yl)methanamine | 536.51 |
| 127 | 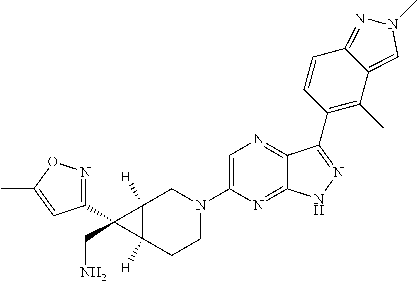 | ((1S,6R,7S)-3-(3-(2,4-dimethyl-2H-indazol-5-yl)-1H- pyrazolo[3,4-b]pyrazin-6-yl)-7-(5-methylisoxazol-3- yl)-3-azabicyclo[4.1.0]heptan-7-yl)methanamine | 469.54 |
| 128 | 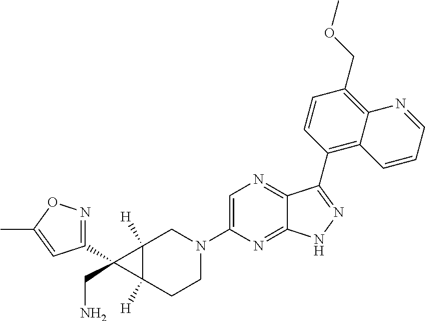 [SMILES: COCc1ccc(-c2n[nH]c3nc(N4CC[C@@H]5[C@H](C4)[C@@]5(CN)c4cc(C)on4)cnc23)c2cccnc12] | ((1S,6R,7S)-3-(3-(8-(methoxymethyl)quinolin-5-yl)- 1H-pyrazolo[3,4-b]pyrazin-6-yl)-7-(5- methylisoxazol-3-yl)-3-azabicyclo[4.1.0]heptan-7- yl)methanamine | 496.56 |
| 129 | 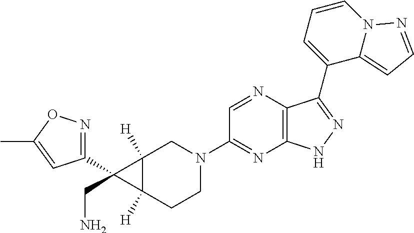 [SMILES: Cc1cc([C@@]2(CN)[C@@H]3CCN(c4cnc5c(-c6cccn7nccc67)n[nH]c5n4)C[C@@H]32)no1] | ((1S,6R,7S)-7-(5-methylisoxazol-3-yl)-3-(3- (pyrazolo[1,5-a]pyridin-4-yl)-1H-pyrazolo[3,4- b]pyrazin-6-yl)-3-azabicyclo[4.1.0]heptan-7- yl)methanamine | 441.49 |
| 130 | 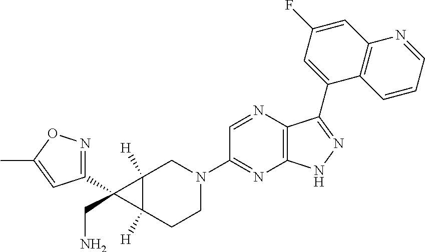 [SMILES: Cc1cc([C@@]2(CN)[C@@H]3CCN(c4cnc5c(-c6cc(F)cc7ncccc67)n[nH]c5n4)C[C@@H]32)no1] | ((1S,6R,7S)-3-(3-(7-fluoroquinolin-5-yl)-1H- pyrazolo[3,4-b]pyrazin-6-yl)-7-(5-methylisoxazol-3- yl)-3-azabicyclo[4.1.0]heptan-7-yl)methanamine | 470.5 |
| 131 | 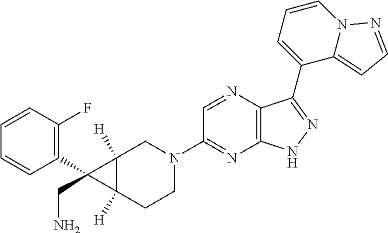 [SMILES: NC[C@]1(c2ccccc2F)[C@@H]2CCN(c3cnc4c(-c5cccn6nccc56)n[nH]c4n3)C[C@@H]21] | ((1S,6R,7R)-7-(2-fluorophenyl)-3-(3-(pyrazolo[1,5- a]pyridin-4-yl)-1H-pyrazolo[3,4-b]pyrazin-6-yl)-3- azabicyclo[4.1.0]heptan-7-yl)methanamine | 454.5 |
| 132 | 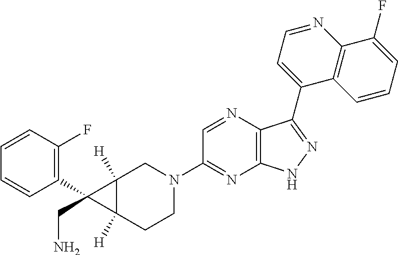 [SMILES: NC[C@]1(c2ccccc2F)[C@@H]2CCN(c3cnc4c(-c5ccnc6c(F)cccc56)n[nH]c4n3)C[C@@H]21] | ((1S,6R,7R)-7-(2-fluorophenyl)-3-(3-(8- fluoroquinolin-4-yl)-1H-pyrazolo[3,4-b]pyrazin-6- yl)-3-azabicyclo[4.1.0]heptan-7-yl)methanamine | 483.52 |
| 133 | 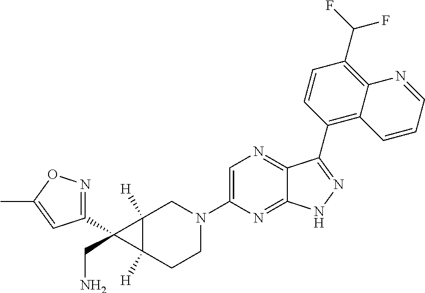 [SMILES: Cc1cc([C@@]2(CN)[C@@H]3CCN(c4cnc5c(-c6ccc(C(F)F)c7ncccc67)n[nH]c5n4)C[C@@H]32)no1] | ((1S,6R,7S)-3-(3-(8-(difluoromethyl)quinolin-5-yl)- 1H-pyrazolo[3,4-b]pyrazin-6-yl)-7-(5- methylisoxazol-3-yl)-3-azabicyclo[4.1.0]heptan-7- yl)methanamine | 502.52 |
| 134 | 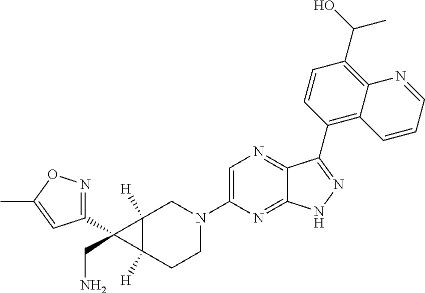 [SMILES: Cc1cc([C@@]2(CN)[C@@H]3CCN(c4cnc5c(-c6ccc(C(C)O)c7ncccc67)n[nH]c5n4)C[C@@H]32)no1] | 1-(5-(6-((1S,6R,7S)-7-(aminomethyl)-7-(5- methylisoxazol-3-yl)-3-azabicyclo[4.1.0]heptan-3- yl)-1H-pyrazolo[3,4-b]pyrazin-3-yl)quinolin-8- yl)ethan-1-ol | 496.56 |
| 135 | 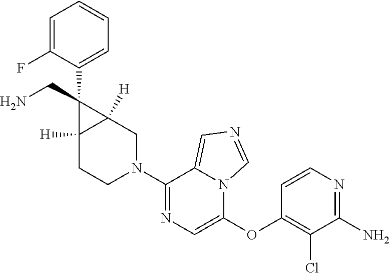 [SMILES: NC[C@]1(c2ccccc2F)[C@@H]2CCN(c3ncc(Oc4ccnc(N)c4Cl)n4cncc34)C[C@@H]21] | 4-((8-((1S,6R,7R)-7-(aminomethyl)-7-(2- fluorophenyl)-3-azabicyclo[4.1.0]heptan-3- yl)imidazo[1,5-a]pyrazin-5-yl)thio)-3-chloropyridin- 2-amine | 496 |
| 136 | 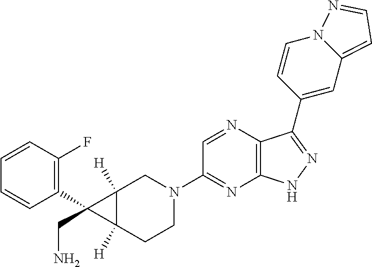 [SMILES: NC[C@]1(c2ccccc2F)[C@@H]2CCN(c3cnc4c(-c5ccn6nccc6c5)n[nH]c4n3)C[C@@H]21] | ((1S,6R,7R)-7-(2-fluorophenyl)-3-(3-(pyrazolo[1,5- a]pyridin-5-yl)-1H-pyrazolo[3,4-b]pyrazin-6-yl)-3- azabicyclo[4.1.0]heptan-7-yl)methanamine | 454.5 |
| 137 | 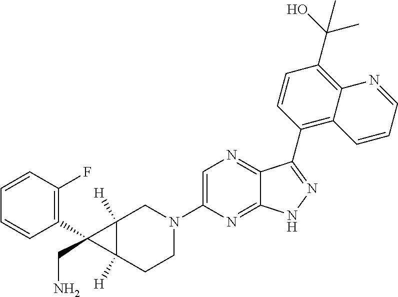 [SMILES: CC(C)(O)c1ccc(-c2n[nH]c3nc(N4CC[C@@H]5[C@H](C4)[C@@]5(CN)c4ccccc4F)cnc23)c2cccnc12] | 2-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-8-yl)propan-2- ol | 523.61 |
| 138 | 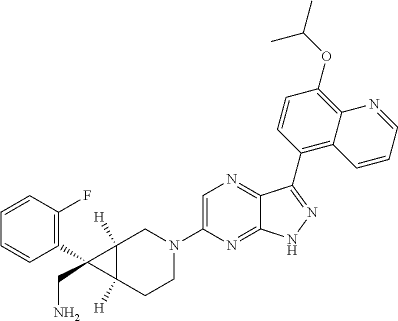 [SMILES: CC(C)Oc1ccc(-c2n[nH]c3nc(N4CC[C@@H]5[C@H](C4)[C@@]5(CN)c4ccccc4F)cnc23)c2cccnc12] | ((1S,6R,7R)-7-(2-fluorophenyl)-3-(3-(8- isopropoxyquinolin-5-yl)-1H-pyrazolo[3,4-b]pyrazin- 6-yl)-3-azabicyclo[4.1.0]heptan-7-yl)methanamine | 523.61 |
| 139 | 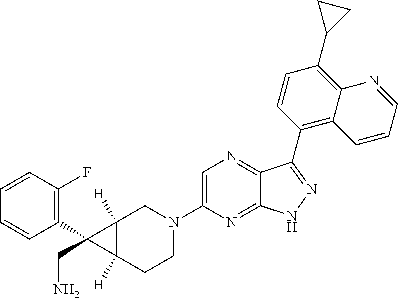 [SMILES: NC[C@]1(c2ccccc2F)[C@@H]2CCN(c3cnc4c(-c5ccc(C6CC6)c6ncccc56)n[nH]c4n3)C[C@@H]21] | ((1S,6R,7R)-3-(3-(8-cyclopropylquinolin-5-yl)-1H- pyrazolo[3,4-b]pyrazin-6-yl)-7-(2-fluorophenyl)-3- azabicyclo[4.1.0]heptan-7-yl)methanamine | 505.59 |
| 140 | 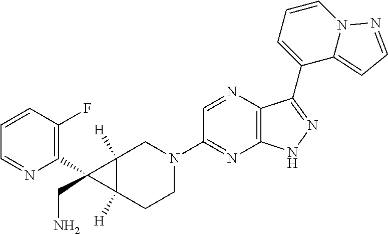 [SMILES: NC[C@]1(c2ncccc2F)[C@@H]2CCN(c3cnc4c(-c5cccn6nccc56)n[nH]c4n3)C[C@@H]21] | ((1S,6R,7S)-7-(3-fluoropyridin-2-yl)-3-(3- (pyrazolo[1,5-a]pyridin-4-yl)-1H-pyrazolo[3,4- b]pyrazin-6-yl)-3-azabicyclo[4.1.0]heptan-7- yl)methanamine | 455.49 |
| 141 | 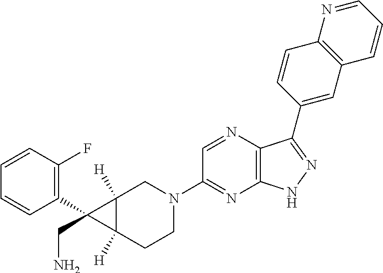 [SMILES: NC[C@]1(c2ccccc2F)[C@@H]2CCN(c3cnc4c(-c5ccc6ncccc6c5)n[nH]c4n3)C[C@@H]21] | ((1S,6R,7R)-7-(2-fluorophenyl)-3-(3-(quinolin-6-yl)- 1H-pyrazolo[3,4-b]pyrazin-6-yl)-3- azabicyclo[4.1.0]heptan-7-yl)methanamine | 465.53 |
| 142 | 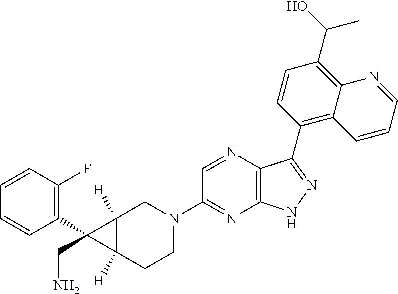 [SMILES: CC(O)c1ccc(-c2n[nH]c3nc(N4CC[C@@H]5[C@H](C4)[C@@]5(CN)c4ccccc4F)cnc23)c2cccnc12] | 1-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-8-yl)ethan-1-ol | 509.58 |
| 143 | 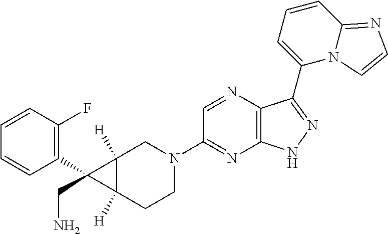 [SMILES: NC[C@]1(c2ccccc2F)[C@@H]2CCN(c3cnc4c(-c5cccc6nccn56)n[nH]c4n3)C[C@@H]21] | ((1S,6R,7R)-7-(2-fluorophenyl)-3-(3-(imidazo[1,2- a]pyridin-5-yl)-1H-pyrazolo[3,4-b]pyrazin-6-yl)-3- azabicyclo[4.1.0]heptan-7-yl)methanamine | 454.5 |
| 144 | 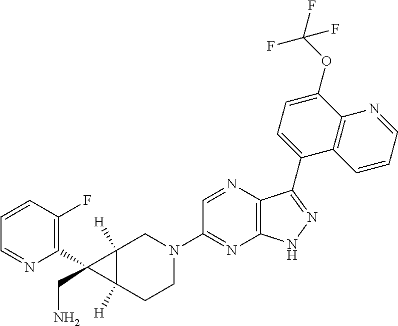 [SMILES: NC[C@]1(c2ncccc2F)[C@@H]2CCN(c3cnc4c(-c5ccc(OC(F)(F)F)c6ncccc56)n[nH]c4n3)C[C@@H]21] | ((1S,6R,7S)-7-(3-fluoropyridin-2-yl)-3-(3-(8- (trifluoromethoxy)quinolin-5-yl)-1H-pyrazolo[3,4- b]pyrazin-6-yl)-3-azabicyclo[4.1.0]heptan-7- yl)methanamine | 550.51 |
| 145 | 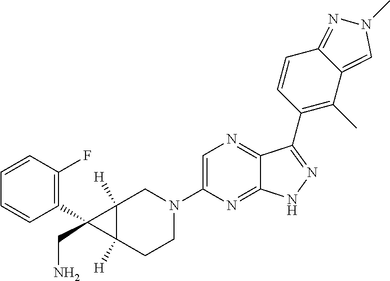 [SMILES: Cc1c(-c2n[nH]c3nc(N4CC[C@@H]5[C@H](C4)[C@@]5(CN)c4ccccc4F)cnc23)ccc2nn(C)cc12] | ((1S,6R,7R)-3-(3-(2,4-dimethyl-2H-indazol-5-yl)- 1H-pyrazolo[3,4-b]pyrazin-6-yl)-7-(2-fluorophenyl)- 3-azabicyclo[4.1.0]heptan-7-yl)methanamine | 482.56 |
| 146 | 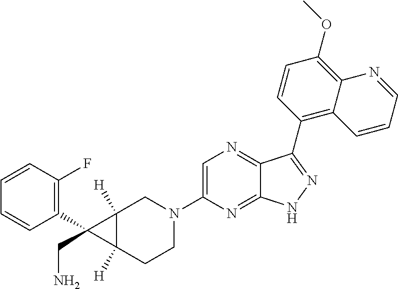 [SMILES: COc1ccc(-c2n[nH]c3nc(N4CC[C@@H]5[C@H](C4)[C@@]5(CN)c4ccccc4F)cnc23)c2cccnc12] | ((1S,6R,7R)-7-(2-fluorophenyl)-3-(3-(8- methoxyquinolin-5-yl)-1H-pyrazolo[3,4-b]pyrazin-6- yl)-3-azabicyclo[4.1.0]heptan-7-yl)methanamine | 495.55 |
| 147 | 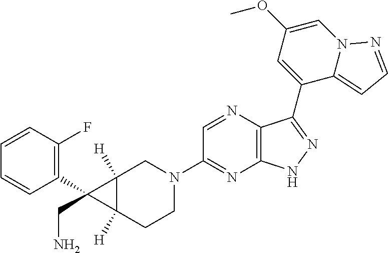 [SMILES: COc1cc(-c2n[nH]c3nc(N4CC[C@@H]5[C@H](C4)[C@@]5(CN)c4ccccc4F)cnc23)c2ccnn2c1] | ((1S,6R,7R)-7-(2-fluorophenyl)-3-(3-(6- methoxypyrazolo[1,5-a]pyridin-4-yl)-1H- pyrazolo[3,4-b]pyrazin-6-yl)-3- azabicyclo[4.1.0]heptan-7-yl)methanamine | 484.53 |
| 148 | 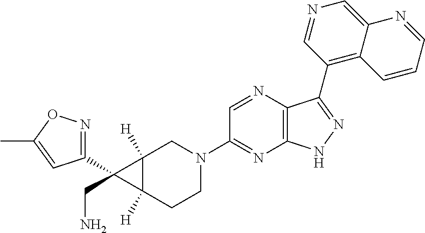 [SMILES: Cc1cc([C@@]2(CN)[C@@H]3CCN(c4cnc5c(-c6cncc7ncccc67)n[nH]c5n4)C[C@@H]32)no1] | ((1S,6R,7S)-3-(3-(1,7-naphthyridin-5-yl)-1H- pyrazolo[3,4-b]pyrazin-6-yl)-7-(5-methylisoxazol-3- yl)-3-azabicyclo[4.1.0]heptan-7-yl)methanamine | 453.5 |
| 149 | 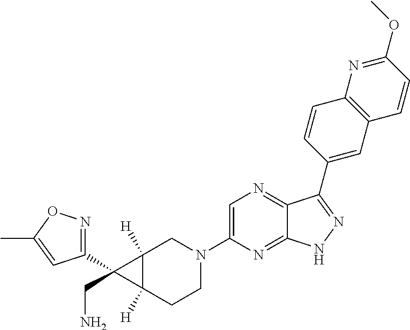 [SMILES: COc1ccc2cc(-c3n[nH]c4nc(N5CC[C@@H]6[C@H](C5)[C@@]6(CN)c5cc(C)on5)cnc34)ccc2n1] | ((1S,6R,7S)-3-(3-(2-methoxyquinolin-6-yl)-1H- pyrazolo[3,4-b]pyrazin-6-yl)-7-(5-methylisoxazol-3- yl)-3-azabicyclo[4.1.0]heptan-7-yl)methanamine | 482.54 |
| 150 | 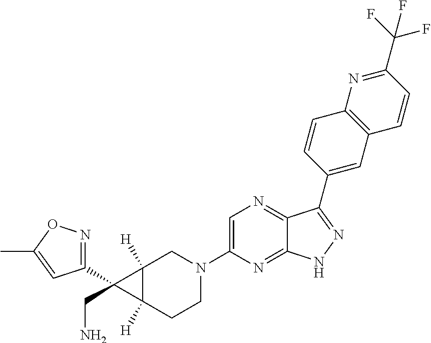 [SMILES: Cc1cc([C@@]2(CN)[C@@H]3CCN(c4cnc5c(-c6ccc7nc(C(F)(F)F)ccc7c6)n[nH]c5n4)C[C@@H]32)no1] | ((1S,6R,7S)-7-(5-methylisoxazol-3-yl)-3-(3-(2- (trifluoromethyl)quinolin-6-yl)-1H-pyrazolo[3,4- b]pyrazin-6-yl)-3-azabicyclo[4.1.0]heptan-7- yl)methanamine | 520.51 |
| 151 | 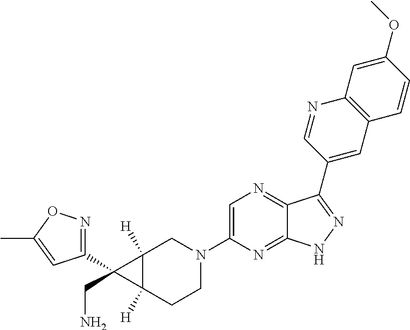 [SMILES: COc1ccc2cc(-c3n[nH]c4nc(N5CC[C@@H]6[C@H](C5)[C@@]6(CN)c5cc(C)on5)cnc34)cnc2c1] | ((1S,6R,7S)-3-(3-(7-methoxyquinolin-3-yl)-1H- pyrazolo[3,4-b]pyrazin-6-yl)-7-(5-methylisoxazol-3- yl)-3-azabicyclo[4.1.0]heptan-7-yl)methanamine | 482.54 |
| 152 | 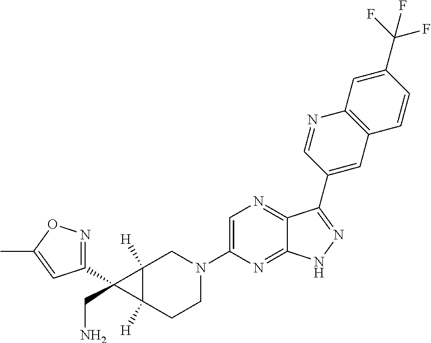 [SMILES: Cc1cc([C@@]2(CN)[C@@H]3CCN(c4cnc5c(-c6cnc7cc(C(F)(F)F)ccc7c6)n[nH]c5n4)C[C@@H]32)no1] | ((1S,6R,7S)-7-(5-methylisoxazol-3-yl)-3-(3-(7- (trifluoromethyl)quinolin-3-yl)-1H-pyrazolo[3,4- b]pyrazin-6-yl)-3-azabicyclo[4.1.0]heptan-7- yl)methanamine | 520.51 |
| 153 | 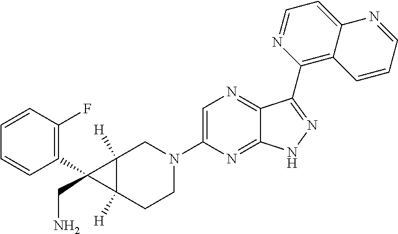 [SMILES: NC[C@]1(c2ccccc2F)[C@@H]2CCN(c3cnc4c(-c5nccc6ncccc56)n[nH]c4n3)C[C@@H]21] | ((1S,6R,7R)-3-(3-(1,6-naphthyridin-5-yl)-1H- pyrazolo[3,4-b]pyrazin-6-yl)-7-(2-fluorophenyl)-3- azabicyclo[4.1.0]heptan-7-yl)methanamine | 466.51 |
| 154 | 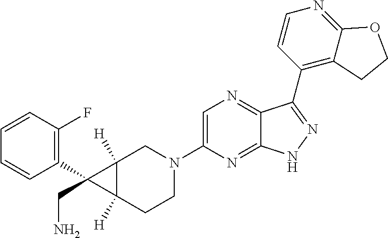 [SMILES: NC[C@]1(c2ccccc2F)[C@@H]2CCN(c3cnc4c(-c5ccnc6c5CCO6)n[nH]c4n3)C[C@@H]21] | ((1S,6R,7R)-3-(3-(2,3-dihydrofuro[2,3-b]pyridin-4- yl)-1H-pyrazolo[3,4-b]pyrazin-6-yl)-7-(2- fluorophenyl)-3-azabicyclo[4.1.0]heptan-7- yl)methanamine | 457.5 |
| 155 | 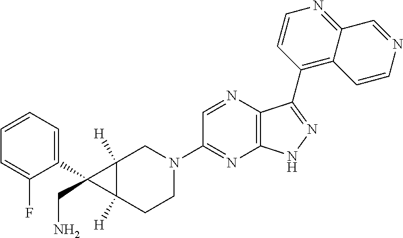 [SMILES: NC[C@]1(c2ccccc2F)[C@@H]2CCN(c3cnc4c(-c5ccnc6cnccc56)n[nH]c4n3)C[C@@H]21] | ((1S,6R,7R)-3-(3-(1,7-naphthyridin-4-yl)-1H- pyrazolo[3,4-b]pyrazin-6-yl)-7-(2-fluorophenyl)-3- azabicyclo[4.1.0]heptan-7-yl)methanamine | 466.51 |
| 156 | 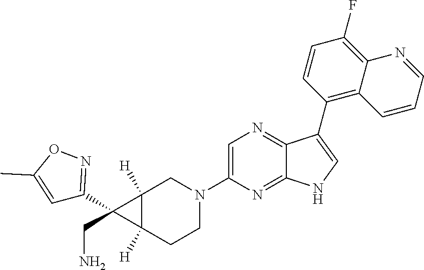 [SMILES: Cc1cc([C@@]2(CN)[C@@H]3CCN(c4cnc5c(-c6ccc(F)c7ncccc67)c[nH]c5n4)C[C@@H]32)no1] | ((1S,6R,7S)-3-(7-(8-fluoroquinolin-5-yl)-5H- pyrrolo[2,3-b]pyrazin-3-yl)-7-(5-methylisoxazol-3- yl)-3-azabicyclo[4.1.0]heptan-7-yl)methanamine | 469.51 |
| 157 | 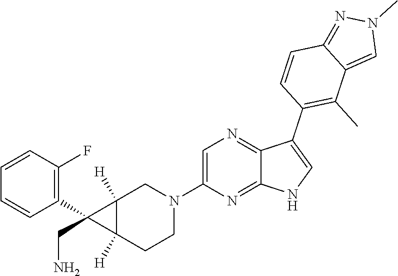 [SMILES: Cc1c(-c2c[nH]c3nc(N4CC[C@@H]5[C@H](C4)[C@@]5(CN)c4ccccc4F)cnc23)ccc2nn(C)cc12] | ((1S,6R,7R)-3-(7-(2,4-dimethyl-2H-indazol-5-yl)- 5H-pyrrolo[2,3-b]pyrazin-3-yl)-7-(2-fluorophenyl)-3- azabicyclo[4.1.0]heptan-7-yl)methanamine | 481.57 |
| 158 | 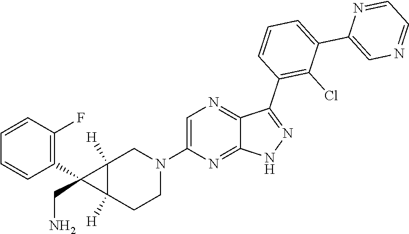 [SMILES: NC[C@]1(c2ccccc2F)[C@@H]2CCN(c3cnc4c(-c5cccc(-c6cnccn6)c5Cl)n[nH]c4n3)C[C@@H]21] | ((1S,6R,7R)-3-(3-(2-chloro-3-(pyrazin-2-yl)phenyl)- 1H-pyrazolo[3,4-b]pyrazin-6-yl)-7-(2-fluorophenyl)- 3-azabicyclo[4.1.0]heptan-7-yl)methanamine | 527 |
| 159 | 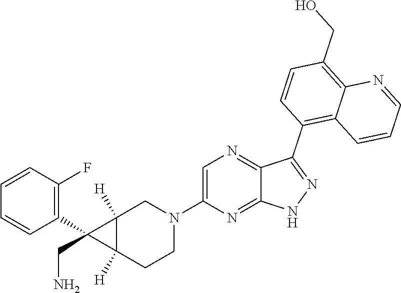 [SMILES: NC[C@]1(c2ccccc2F)[C@@H]2CCN(c3cnc4c(-c5ccc(CO)c6ncccc56)n[nH]c4n3)C[C@@H]21] | (5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-8-yl)methanol | 495.55 |
| 160 | 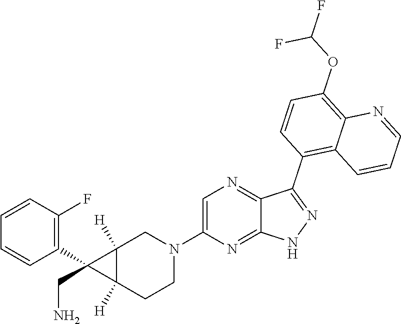 [SMILES: NC[C@]1(c2ccccc2F)[C@@H]2CCN(c3cnc4c(-c5ccc(OC(F)F)c6ncccc56)n[nH]c4n3)C[C@@H]21] | ((1S,6R,7R)-3-(3-(8-(difluoromethoxy)quinolin-5- yl)-1H-pyrazolo[3,4-b]pyrazin-6-yl)-7-(2- fluorophenyl)-3-azabicyclo[4.1.0]heptan-7- yl)methanamine | 531.53 |
| 161 | 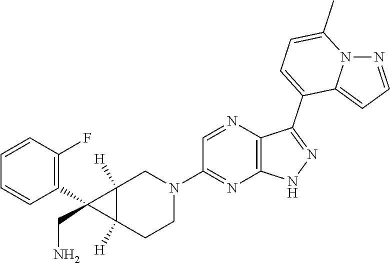 [SMILES: Cc1ccc(-c2n[nH]c3nc(N4CC[C@@H]5[C@H](C4)[C@@]5(CN)c4ccccc4F)cnc23)c2ccnn12] | ((1S,6R,7R)-7-(2-fluorophenyl)-3-(3-(7- methylpyrazolo[1,5-a]pyridin-4-yl)-1H-pyrazolo[3,4- b]pyrazin-6-yl)-3-azabicyclo[4.1.0]heptan-7- yl)methanamine | 468.53 |
| 162 | 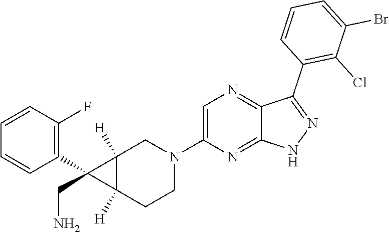 [SMILES: NC[C@]1(c2ccccc2F)[C@@H]2CCN(c3cnc4c(-c5cccc(Br)c5Cl)n[nH]c4n3)C[C@@H]21] | ((1S,6R,7R)-3-(3-(3-bromo-2-chlorophenyl)-1H- pyrazolo[3,4-b]pyrazin-6-yl)-7-(2-fluorophenyl)-3- azabicyclo[4.1.0]heptan-7-yl)methanamine | 527.82 |
| 163 | 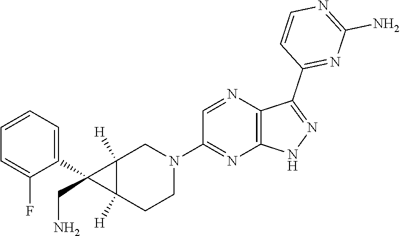 [SMILES: NC[C@]1(c2ccccc2F)[C@@H]2CCN(c3cnc4c(-c5ccnc(N)n5)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)pyrimidin-2-amine | 431.47 |
| 164 | 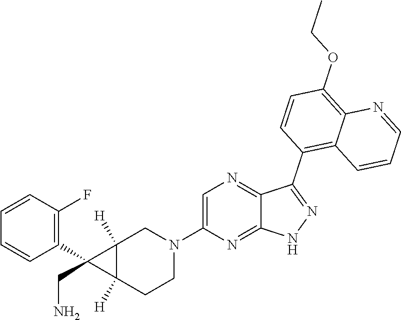 [SMILES: CCOc1ccc(-c2n[nH]c3nc(N4CC[C@@H]5[C@H](C4)[C@@]5(CN)c4ccccc4F)cnc23)c2cccnc12] | ((1S,6R,7R)-3-(3-(8-ethoxyquinolin-5-yl)-1H- pyrazolo[3,4-b]pyrazin-6-yl)-7-(2-fluorophenyl)-3- azabicyclo[4.1.0]heptan-7-yl)methanamine | 509.58 |
| 165 | 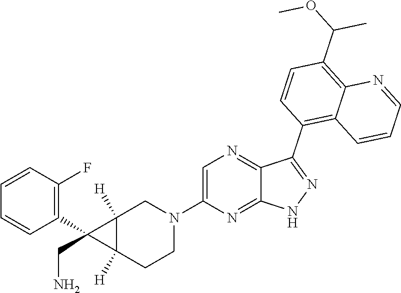 [SMILES: COC(C)c1ccc(-c2n[nH]c3nc(N4CC[C@@H]5[C@H](C4)[C@@]5(CN)c4ccccc4F)cnc23)c2cccnc12] | ((1S,6R,7R)-7-(2-fluorophenyl)-3-(3-(8-(1- methoxyethyl)quinolin-5-yl)-1H-pyrazolo[3,4- b]pyrazin-6-yl)-3-azabicyclo[4.1.0]heptan-7- yl)methanamine | 523.61 |
| 166 | 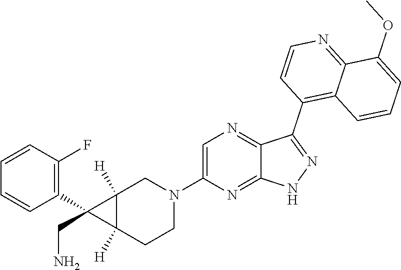 [SMILES: COc1cccc2c(-c3n[nH]c4nc(N5CC[C@@H]6[C@H](C5)[C@@]6(CN)c5ccccc5F)cnc34)ccnc12] | ((1S,6R,7R)-7-(2-fluorophenyl)-3-(3-(8- methoxyquinolin-4-yl)-1H-pyrazolo[3,4-b]pyrazin-6- yl)-3-azabicyclo[4.1.0]heptan-7-yl)methanamine | 495.55 |
| 167 | 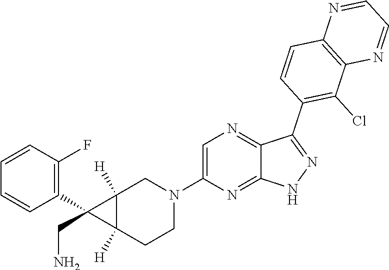 [SMILES: NC[C@]1(c2ccccc2F)[C@@H]2CCN(c3cnc4c(-c5ccc6nccnc6c5Cl)n[nH]c4n3)C[C@@H]21] | ((1S,6R,7R)-3-(3-(5-chloroquinoxalin-6-yl)-1H- pyrazolo[3,4-b]pyrazin-6-yl)-7-(2-fluorophenyl)-3- azabicyclo[4.1.0]heptan-7-yl)methanamine | 500.96 |
| 168 | 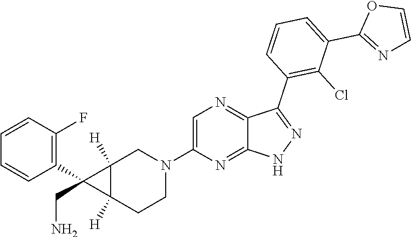 [SMILES: NC[C@]1(c2ccccc2F)[C@@H]2CCN(c3cnc4c(-c5cccc(-c6ncco6)c5Cl)n[nH]c4n3)C[C@@H]21] | ((1S,6R,7R)-3-(3-(2-chloro-3-(oxazol-2-yl)phenyl)- 1H-pyrazolo[3,4-b]pyrazin-6-yl)-7-(2-fluorophenyl)- 3-azabicyclo[4.1.0]heptan-7-yl)methanamine | 515.97 |
| 169 | 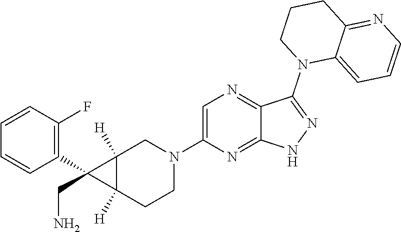 [SMILES: NC[C@]1(c2ccccc2F)[C@@H]2CCN(c3cnc4c(N5CCCc6ncccc65)n[nH]c4n3)C[C@@H]21] | ((1S,6R,7R)-3-(3-(3,4-dihydro-1,5-naphthyridin- 1(2H)-yl)-1H-pyrazolo[3,4-b]pyrazin-6-yl)-7-(2- fluorophenyl)-3-azabicyclo[4.1.0]heptan-7- yl)methanamine | 470.55 |
| 170 | 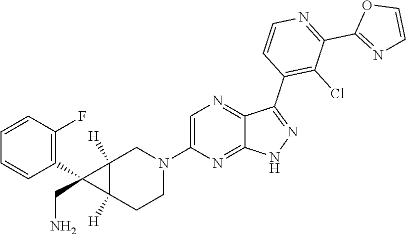 [SMILES: NC[C@]1(c2ccccc2F)[C@@H]2CCN(c3cnc4c(-c5ccnc(-c6ncco6)c5Cl)n[nH]c4n3)C[C@@H]21] | ((1S,6R,7R)-3-(3-(3-chloro-2-(oxazol-2-yl)pyridin-4- yl)-1H-pyrazolo[3,4-b]pyrazin-6-yl)-7-(2- fluorophenyl)-3-azabicyclo[4.1.0]heptan-7- yl)methanamine | 516.96 |
| 171 | 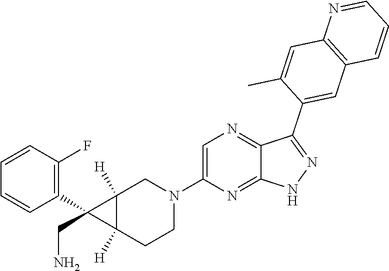 [SMILES: Cc1cc2ncccc2cc1-c1n[nH]c2nc(N3CC[C@@H]4[C@H](C3)[C@@]4(CN)c3ccccc3F)cnc12] | ((1S,6R,7R)-7-(2-fluorophenyl)-3-(3-(7- methylquinolin-6-yl)-1H-pyrazolo[3,4-b]pyrazin-6- yl)-3-azabicyclo[4.1.0]heptan-7-yl)methanamine | 479.55 |
| 172 | 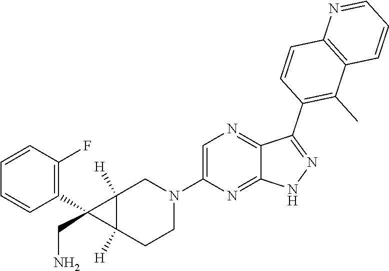 [SMILES: Cc1c(-c2n[nH]c3nc(N4CC[C@@H]5[C@H](C4)[C@@]5(CN)c4ccccc4F)cnc23)ccc2ncccc12] | ((1S,6R,7R)-7-(2-fluorophenyl)-3-(3-(5- methylquinolin-6-yl)-1H-pyrazolo[3,4-b]pyrazin-6- yl)-3-azabicyclo[4.1.0]heptan-7-yl)methanamine | 479.55 |
| 173 | 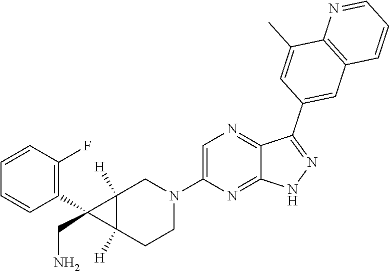 [SMILES: Cc1cc(-c2n[nH]c3nc(N4CC[C@@H]5[C@H](C4)[C@@]5(CN)c4ccccc4F)cnc23)cc2cccnc12] | ((1S,6R,7R)-7-(2-fluorophenyl)-3-(3-(8- methylquinolin-6-yl)-1H-pyrazolo[3,4-b]pyrazin-6- yl)-3-azabicyclo[4.1.0]heptan-7-yl)methanamine | 479.55 |
| 174 | 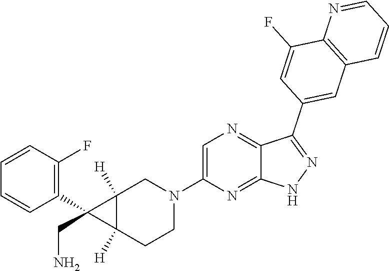 [SMILES: NC[C@]1(c2ccccc2F)[C@@H]2CCN(c3cnc4c(-c5cc(F)c6ncccc6c5)n[nH]c4n3)C[C@@H]21] | ((1S,6R,7R)-7-(2-fluorophenyl)-3-(3-(8- fluoroquinolin-6-yl)-1H-pyrazolo[3,4-b]pyrazin-6- yl)-3-azabicyclo[4.1.0]heptan-7-yl)methanamine | 483.52 |
| 175 | 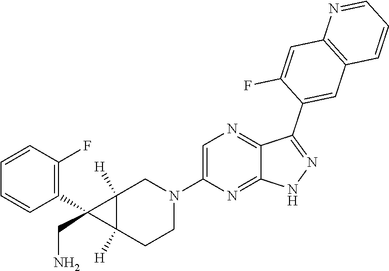 [SMILES: NC[C@]1(c2ccccc2F)[C@@H]2CCN(c3cnc4c(-c5cc6cccnc6cc5F)n[nH]c4n3)C[C@@H]21] | ((1S,6R,7R)-7-(2-fluorophenyl)-3-(3-(7- fluoroquinolin-6-yl)-1H-pyrazolo[3,4-b]pyrazin-6- yl)-3-azabicyclo[4.1.0]heptan-7-yl)methanamine | 483.52 |
| 176 | 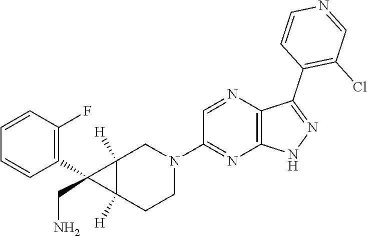 [SMILES: NC[C@]1(c2ccccc2F)[C@@H]2CCN(c3cnc4c(-c5ccncc5Cl)n[nH]c4n3)C[C@@H]21] | ((1S,6R,7R)-3-(3-(3-chloropyridin-4-yl)-1H- pyrazolo[3,4-b]pyrazin-6-yl)-7-(2-fluorophenyl)-3- azabicyclo[4.1.0]heptan-7-yl)methanamine | 449.91 |
| 177 | 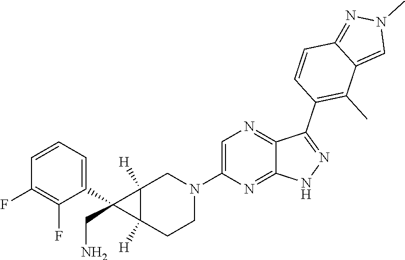 [SMILES: Cc1c(-c2n[nH]c3nc(N4CC[C@@H]5[C@H](C4)[C@@]5(CN)c4cccc(F)c4F)cnc23)ccc2nn(C)cc12] | ((1S,6R,7R)-7-(2,3-difluorophenyl)-3-(3-(2,4- dimethyl-2H-indazol-5-yl)-1H-pyrazolo[3,4- b]pyrazin-6-yl)-3-azabicyclo[4.1.0]heptan-7- yl)methanamine | 500.55 |
| 178 | 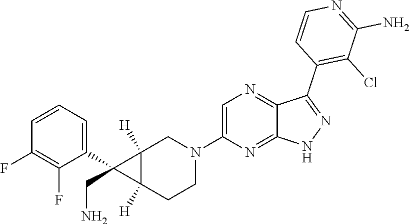 [SMILES: NC[C@]1(c2cccc(F)c2F)[C@@H]2CCN(c3cnc4c(-c5ccnc(N)c5Cl)n[nH]c4n3)C[C@@H]21] | 4-(6-((1S,6R,7R)-7-(aminomethyl)-7-(2,3- difluorophenyl)-3-azabicyclo[4.1.0]heptan-3-yl)-1H- pyrazolo[3,4-b]pyrazin-3-yl)-3-chloropyridin-2- amine | 482.92 |
| 179 | 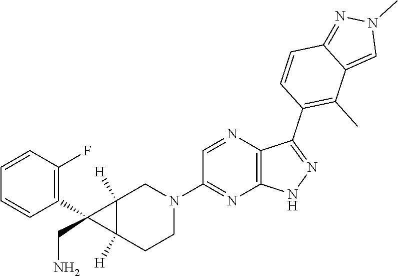 [SMILES: Cc1c(-c2n[nH]c3nc(N4CC[C@@H]5[C@H](C4)[C@@]5(CN)c4ccccc4F)cnc23)ccc2nn(C)cc12] | ((1S,6R,7S)-3-(3-(2,4-dimethyl-2H-indazol-5-yl)-1H- pyrazolo[3,4-b]pyrazin-6-yl)-7-(3-fluoropyridin-2- yl)-3-azabicyclo[4.1.0]heptan-7-yl)methanamine | 483.54 |
| 180 | 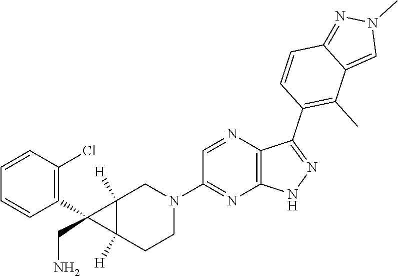 [SMILES: Cc1c(-c2n[nH]c3nc(N4CC[C@@H]5[C@H](C4)[C@@]5(CN)c4ccccc4Cl)cnc23)ccc2nn(C)cc12] | ((1S,6R,7R)-7-(2-chlorophenyl)-3-(3-(2,4-dimethyl- 2H-indazol-5-yl)-1H-pyrazolo[3,4-b]pyrazin-6-yl)-3- azabicyclo[4.1.0]heptan-7-yl)methanamine | 499.01 |
| 181 | 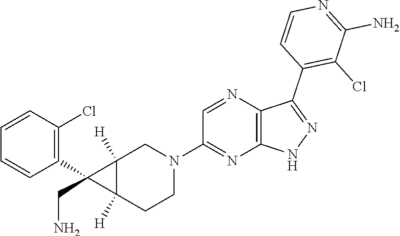 [SMILES: NC[C@]1(c2ccccc2Cl)[C@@H]2CCN(c3cnc4c(-c5ccnc(N)c5Cl)n[nH]c4n3)C[C@@H]21] | 4-(6-((1S,6R,7R)-7-(aminomethyl)-7-(2- chlorophenyl)-3-azabicyclo[4.1.0]heptan-3-yl)-1H- pyrazolo[3,4-b]pyrazin-3-yl)-3-chloropyridin-2- amine | 481.38 |
| 182 | 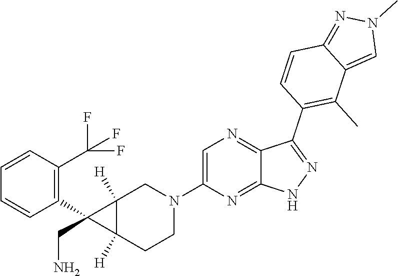 [SMILES: Cc1c(-c2n[nH]c3nc(N4CC[C@@H]5[C@H](C4)[C@@]5(CN)c4ccccc4C(F)(F)F)cnc23)ccc2nn(C)cc12] | ((1S,6R,7R)-3-(3-(2,4-dimethyl-2H-indazol-5-yl)- 1H-pyrazolo[3,4-b]pyrazin-6-yl)-7-(2- (trifluoromethyl)phenyl)-3-azabicyclo[4.1.0]heptan- 7-yl)methanamine | 532.56 |
| 183 | 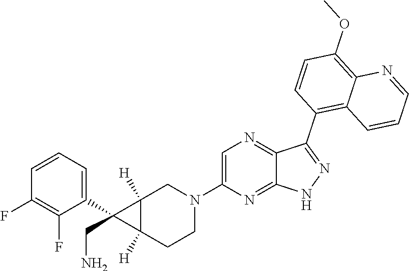 [SMILES: COc1ccc(-c2n[nH]c3nc(N4CC[C@@H]5[C@H](C4)[C@@]5(CN)c4cccc(F)c4F)cnc23)c2cccnc12] | ((1S,6R,7R)-7-(2,3-difluorophenyl)-3-(3-(8- methoxyquinolin-5-yl)-1H-pyrazolo[3,4-b]pyrazin-6- yl)-3-azabicyclo[4.1.0]heptan-7-yl)methanamine | 513.54 |
| 184 | 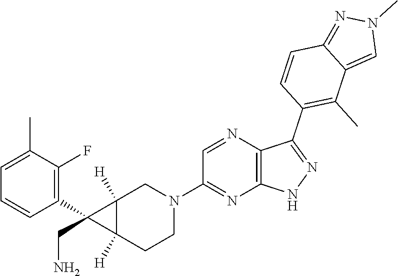 [SMILES: Cc1cccc([C@@]2(CN)[C@@H]3CCN(c4cnc5c(-c6ccc7nn(C)cc7c6C)n[nH]c5n4)C[C@@H]32)c1F] | ((1S,6R,7R)-3-(3-(2,4-dimethyl-2H-indazol-5-yl)- 1H-pyrazolo[3,4-b]pyrazin-6-yl)-7-(2-fluoro-3- methylphenyl)-3-azabicyclo[4.1.0]heptan-7- yl)methanamine | 496.58 |
| 185 | 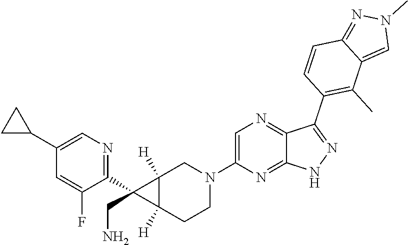 [SMILES: Cc1c(-c2n[nH]c3nc(N4CC[C@@H]5[C@H](C4)[C@@]5(CN)c4ncc(C5CC5)cc4F)cnc23)ccc2nn(C)cc12] | ((1S,6R,7S)-7-(5-cyclopropyl-3-fluoropyridin-2-yl)- 3-(3-(2,4-dimethyl-2H-indazol-5-yl)-1H- pyrazolo[3,4-b]pyrazin-6-yl)-3- azabicyclo[4.1.0]heptan-7-yl)methanamine | 523.61 |
| 186 | 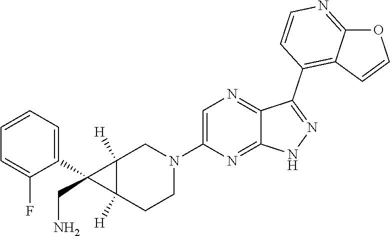 [SMILES: NC[C@]1(c2ccccc2F)[C@@H]2CCN(c3cnc4c(-c5ccnc6occc56)n[nH]c4n3)C[C@@H]21] | ((1S,6R,7R)-7-(2-fluorophenyl)-3-(3-(furo[2,3- b]pyridin-4-yl)-1H-pyrazolo[3,4-b]pyrazin-6-yl)-3- azabicyclo[4.1.0]heptan-7-yl)methanamine | 455.49 |
| 187 | 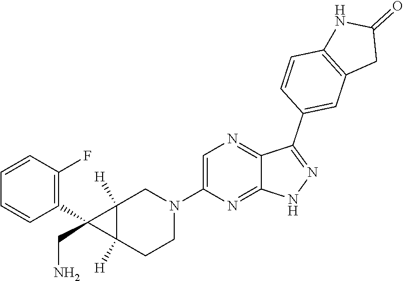 [SMILES: NC[C@]1(c2ccccc2F)[C@@H]2CCN(c3cnc4c(-c5ccc6c(c5)CC(=O)N6)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)indolin-2-one | 469.51 |
| 188 | 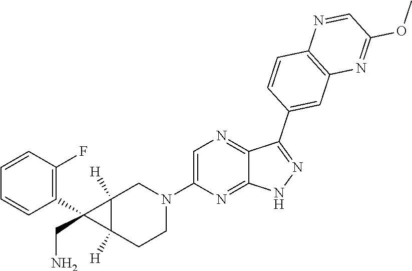 [SMILES: COc1cnc2ccc(-c3n[nH]c4nc(N5CC[C@@H]6[C@H](C5)[C@@]6(CN)c5ccccc5F)cnc34)cc2n1] | ((1S,6R,7R)-7-(2-fluorophenyl)-3-(3-(3- methoxyquinoxalin-6-yl)-1H-pyrazolo[3,4-b]pyrazin- 6-yl)-3-azabicyclo[4.1.0]heptan-7-yl)methanamine | 496.54 |
| 189 | 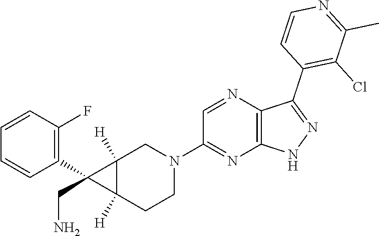 [SMILES: Cc1nccc(-c2n[nH]c3nc(N4CC[C@@H]5[C@H](C4)[C@@]5(CN)c4ccccc4F)cnc23)c1Cl] | ((1S,6R,7R)-3-(3-(3-chloro-2-methylpyridin-4-yl)- 1H-pyrazolo[3,4-b]pyrazin-6-yl)-7-(2-fluorophenyl)- 3-azabicyclo[4.1.0]heptan-7-yl)methanamine | 463.94 |
| 190 | 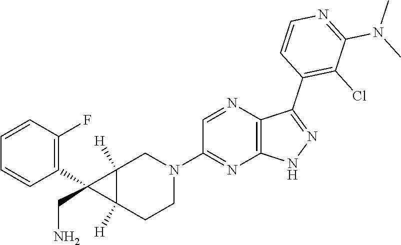 [SMILES: CN(C)c1nccc(-c2n[nH]c3nc(N4CC[C@@H]5[C@H](C4)[C@@]5(CN)c4ccccc4F)cnc23)c1Cl] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-N,N- dimethylpyridin-2-amine | 492.98 |
| 191 | 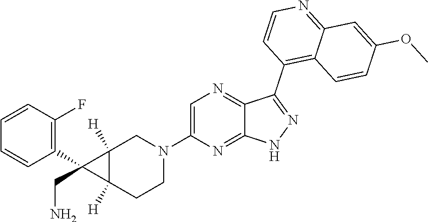 [SMILES: COc1ccc2c(-c3n[nH]c4nc(N5CC[C@@H]6[C@H](C5)[C@@]6(CN)c5ccccc5F)cnc34)ccnc2c1] | ((1S,6R,7R)-7-(2-fluorophenyl)-3-(3-(7- methoxyquinolin-4-yl)-1H-pyrazolo[3,4-b]pyrazin-6- yl)-3-azabicyclo[4.1.0]heptan-7-yl)methanamine | 495.55 |
| 192 | 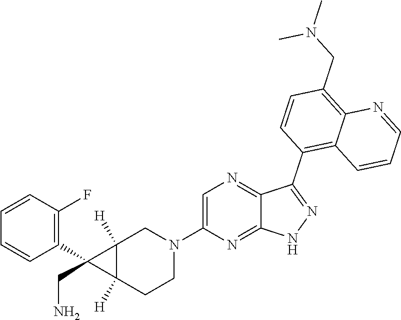 [SMILES: CN(C)Cc1ccc(-c2n[nH]c3nc(N4CC[C@@H]5[C@H](C4)[C@@]5(CN)c4ccccc4F)cnc23)c2cccnc12] | 1-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-8-yl)-N,N- dimethylmethanamine | 522.62 |
| 193 | 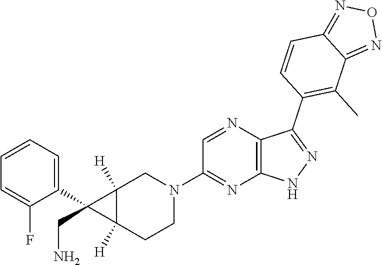 [SMILES: Cc1c(-c2n[nH]c3nc(N4CC[C@@H]5[C@H](C4)[C@@]5(CN)c4ccccc4F)cnc23)ccc2nonc12] | ((1S,6R,7R)-7-(2-fluorophenyl)-3-(3-(4- methylbenzo[c][1,2,5]oxadiazol-5-yl)-1H- pyrazolo[3,4-b]pyrazin-6-yl)-3- azabicyclo[4.1.0]heptan-7-yl)methanamine | 470.5 |
| 194 | 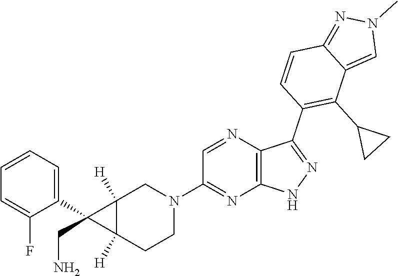 [SMILES: Cn1cc2c(C3CC3)c(-c3n[nH]c4nc(N5CC[C@@H]6[C@H](C5)[C@@]6(CN)c5ccccc5F)cnc34)ccc2n1] | ((1S,6R,7R)-3-(3-(4-cyclopropyl-2-methyl-2H- indazol-5-yl)-1H-pyrazolo[3,4-b]pyrazin-6-yl)-7-(2- fluorophenyl)-3-azabicyclo[4.1.0]heptan-7- yl)methanamine | 508.59 |
| 195 | 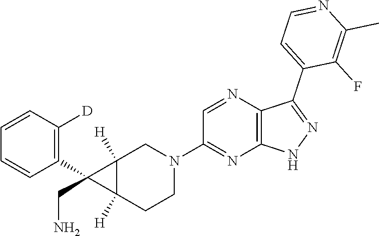 [SMILES: [2H]c1ccccc1[C@@]1(CN)[C@@H]2CCN(c3cnc4c(-c5ccnc(C)c5F)n[nH]c4n3)C[C@@H]21] | ((1S,6R,7R)-3-(3-(3-fluoro-2-methylpyridin-4-yl)- 1H-pyrazolo[3,4-b]pyrazin-6-yl)-7-(2-fluorophenyl)- 3-azabicyclo[4.1.0]heptan-7-yl)methanamine | 447.48 |
| 196 | 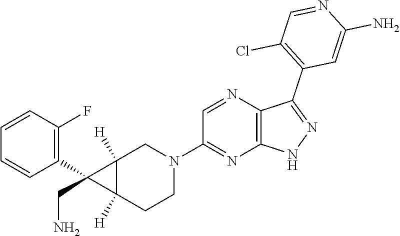 [SMILES: NC[C@]1(c2ccccc2F)[C@@H]2CCN(c3cnc4c(-c5cc(N)ncc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-chloropyridin-2- amine | 464.93 |
| 197 | 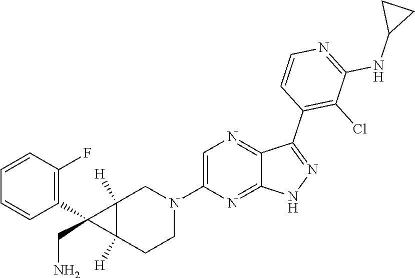 [SMILES: NC[C@]1(c2ccccc2F)[C@@H]2CCN(c3cnc4c(-c5ccnc(NC6CC6)c5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-N- cyclopropylpyridin-2-amine | 504.99 |
| 198 | 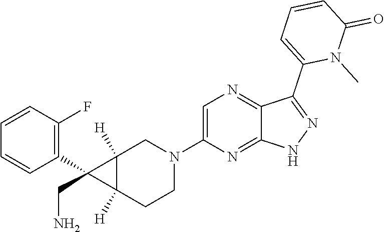 [SMILES: Cn1c(-c2n[nH]c3nc(N4CC[C@@H]5[C@H](C4)[C@@]5(CN)c4ccccc4F)cnc23)cccc1=O] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-methylpyridin-2(1H)- one | 445.49 |
| 199 | 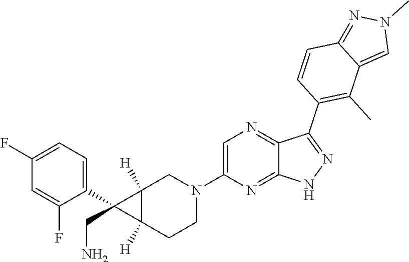 [SMILES: Cc1c(-c2n[nH]c3nc(N4CC[C@@H]5[C@H](C4)[C@@]5(CN)c4ccc(F)cc4F)cnc23)ccc2nn(C)cc12] | ((1S,6R,7R)-7-(2,4-difluorophenyl)-3-(3-(2,4- dimethyl-2H-indazol-5-yl)-1H-pyrazolo[3,4- b]pyrazin-6-yl)-3-azabicyclo[4.1.0]heptan-7- yl)methanamine | 500.55 |
| 200 | 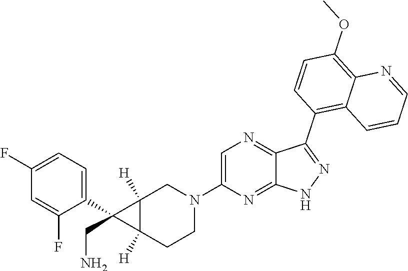 [SMILES: COc1ccc(-c2n[nH]c3nc(N4CC[C@@H]5[C@H](C4)[C@@]5(CN)c4ccc(F)cc4F)cnc23)c2cccnc12] | ((1S,6R,7R)-7-(2,4-difluorophenyl)-3-(3-(8- methoxyquinolin-5-yl)-1H-pyrazolo[3,4-b]pyrazin-6- yl)-3-azabicyclo[4.1.0]heptan-7-yl)methanamine | 513.54 |
| 201 | 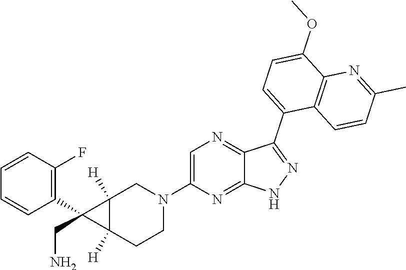 [SMILES: COc1ccc(-c2n[nH]c3nc(N4CC[C@@H]5[C@H](C4)[C@@]5(CN)c4ccccc4F)cnc23)c2ccc(C)nc12] | ((1S,6R,7R)-7-(2-fluorophenyl)-3-(3-(8-methoxy-2- methylquinolin-5-yl)-1H-pyrazolo[3,4-b]pyrazin-6- yl)-3-azabicyclo[4.1.0]heptan-7-yl)methanamine | 509.58 |
| 202 | 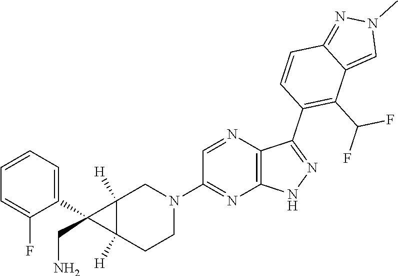 [SMILES: Cn1cc2c(C(F)F)c(-c3n[nH]c4nc(N5CC[C@@H]6[C@H](C5)[C@@]6(CN)c5ccccc5F)cnc34)ccc2n1] | ((1S,6R,7R)-3-(3-(4-(difluoromethyl)-2-methyl-2H- indazol-5-yl)-1H-pyrazolo[3,4-b]pyrazin-6-yl)-7-(2- fluorophenyl)-3-azabicyclo[4.1.0]heptan-7- yl)methanamine | 518.54 |
| 203 | 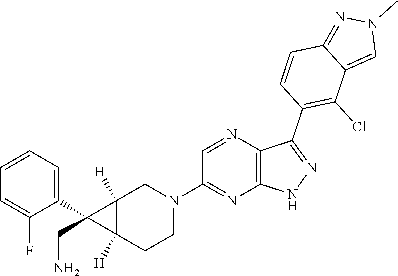 [SMILES: Cn1cc2c(Cl)c(-c3n[nH]c4nc(N5CC[C@@H]6[C@H](C5)[C@@]6(CN)c5ccccc5F)cnc34)ccc2n1] | ((1S,6R,7R)-3-(3-(4-chloro-2-methyl-2H-indazol-5- yl)-1H-pyrazolo[3,4-b]pyrazin-6-yl)-7-(2- fluorophenyl)-3-azabicyclo[4.1.0]heptan-7- yl)methanamine | 502.97 |
| 204 | 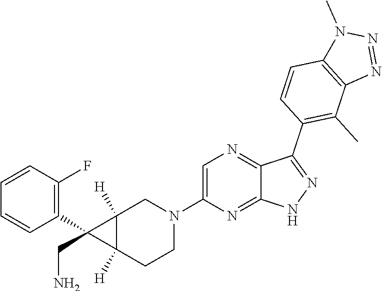 [SMILES: Cc1c(-c2n[nH]c3nc(N4CC[C@@H]5[C@H](C4)[C@@]5(CN)c4ccccc4F)cnc23)ccc2c1nnn2C] | ((1S,6R,7R)-3-(3-(1,4-dimethyl-1H- benzo[d][1,2,3]triazol-5-yl)-1H-pyrazolo[3,4- b]pyrazin-6-yl)-7-(2-fluorophenyl)-3- azabicyclo[4.1.0]heptan-7-yl)methanamine | 483.54 |
| 205 | 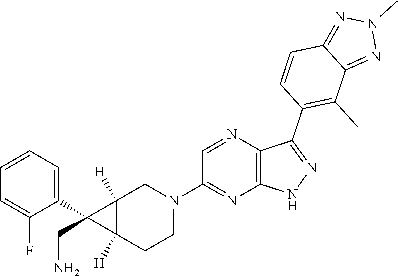 [SMILES: Cc1c(-c2n[nH]c3nc(N4CC[C@@H]5[C@H](C4)[C@@]5(CN)c4ccccc4F)cnc23)ccc2nn(C)nc12] | ((1S,6R,7R)-3-(3-(2,4-dimethyl-2H- benzo[d][1,2,3]triazol-5-yl)-1H-pyrazolo[3,4- b]pyrazin-6-yl)-7-(2-fluorophenyl)-3- azabicyclo[4.1.0]heptan-7-yl)methanamine | 483.54 |
| 206 | 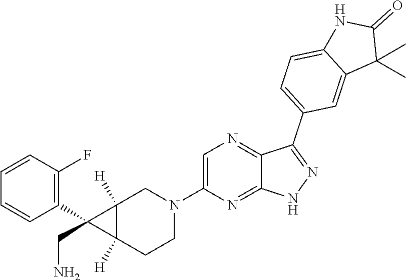 [SMILES: CC1(C)C(=O)Nc2ccc(-c3n[nH]c4nc(N5CC[C@@H]6[C@H](C5)[C@@]6(CN)c5ccccc5F)cnc34)cc21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,3-dimethylindolin-2- one | 497.57 |
| 207 | 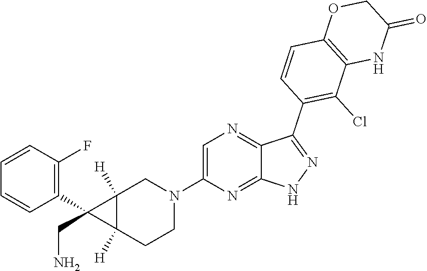 [SMILES: NC[C@]1(c2ccccc2F)[C@@H]2CCN(c3cnc4c(-c5ccc6c(c5Cl)NC(=O)CO6)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-chloro-2H- benzo[b][1,4]oxazin-3(4H)-one | 519.96 |
| 208 | 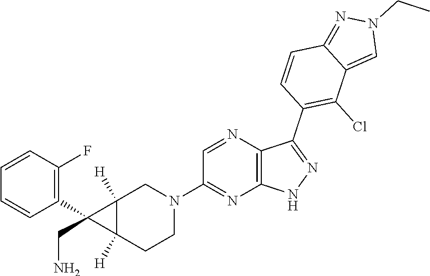 [SMILES: CCn1cc2c(Cl)c(-c3n[nH]c4nc(N5CC[C@@H]6[C@H](C5)[C@@]6(CN)c5ccccc5F)cnc34)ccc2n1] | ((1S,6R,7R)-3-(3-(4-chloro-2-ethyl-2H-indazol-5-yl)- 1H-pyrazolo[3,4-b]pyrazin-6-yl)-7-(2-fluorophenyl)- 3-azabicyclo[4.1.0]heptan-7-yl)methanamine | 517 |
| 209 | 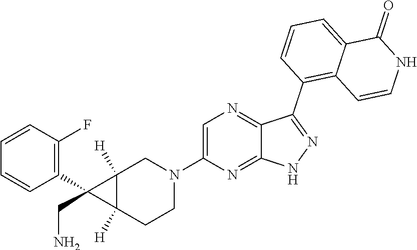 [SMILES: NC[C@]1(c2ccccc2F)[C@@H]2CCN(c3cnc4c(-c5cccc6c(=O)[nH]ccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)isoquinolin-1(2H)-one | 481.53 |
| 210 | 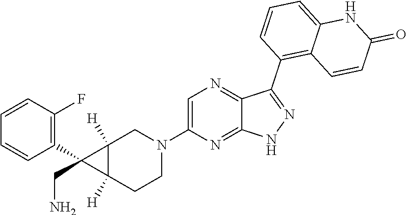 [SMILES: NC[C@]1(c2ccccc2F)[C@@H]2CCN(c3cnc4c(-c5cccc6[nH]c(=O)ccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-2(1H)-one | 481.53 |
| 211 | 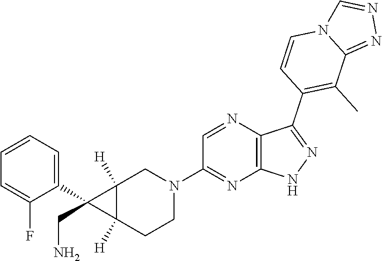 [SMILES: Cc1c(-c2n[nH]c3nc(N4CC[C@@H]5[C@H](C4)[C@@]5(CN)c4ccccc4F)cnc23)ccn2cnnc12] | ((1S,6R,7R)-7-(2-fluorophenyl)-3-(3-(8-methyl- [1,2,4]triazolo[4,3-a]pyridin-7-yl)-1H-pyrazolo[3,4- b]pyrazin-6-yl)-3-azabicyclo[4.1.0]heptan-7- yl)methanamine | 469.52 |
| 212 | 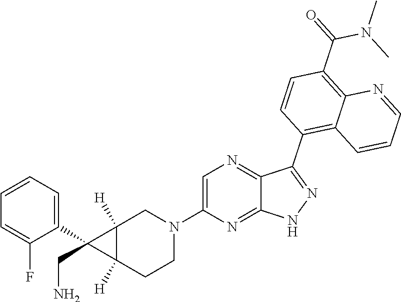 [SMILES: CN(C)C(=O)c1ccc(-c2n[nH]c3nc(N4CC[C@@H]5[C@H](C4)[C@@]5(CN)c4ccccc4F)cnc23)c2cccnc12] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-N,N-dimethylquinoline- 8-carboxamide | 536.6 |
| 213 | 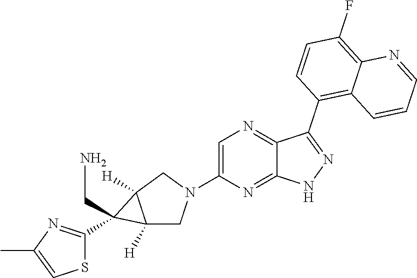 [SMILES: Cc1csc([C@@]2(CN)[C@@H]3CN(c4cnc5c(-c6ccc(F)c7ncccc67)n[nH]c5n4)C[C@@H]32)n1] | ((1R,5S,6r)-3-(3-(8-fluorodecahydroquinolin-5-yl)- 4,7-dihydro-1H-pyrazolo[3,4-b]pyrazin-6-yl)-6-(4- methylthiazol-2-yl)-3-azabicyclo[3.1.0]hexan-6- yl)methanamine | 472.54 |
| 214 | 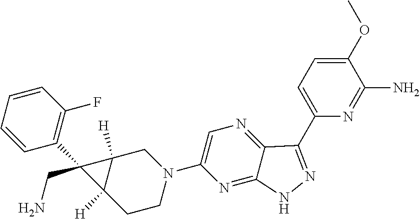 [SMILES: COc1ccc(-c2n[nH]c3nc(N4CC[C@@H]5[C@H](C4)[C@@]5(CN)c4ccccc4F)cnc23)nc1N] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-methoxypyridin-2- amine | 460.51 |
| 215 | 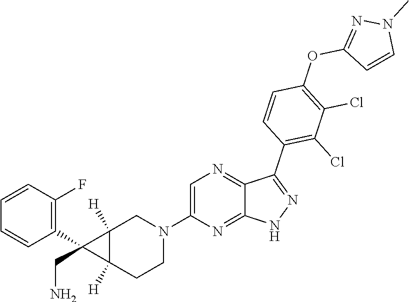 [SMILES: Cn1ccc(Oc2ccc(-c3n[nH]c4nc(N5CC[C@@H]6[C@H](C5)[C@@]6(CN)c5ccccc5F)cnc34)c(Cl)c2Cl)n1] | ((1S,6R,7R)-3-(3-(2,3-dichloro-4-((1-methyl-1H- pyrazol-3-yl)oxy)phenyl)-1H-pyrazolo[3,4-b]pyrazin- 6-yl)-7-(2-fluorophenyl)-3-azabicyclo[4.1.0]heptan- 7-yl)methanamine | 579.46 |
| 216 | 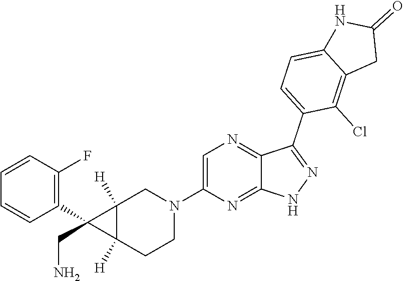 [SMILES: NC[C@]1(c2ccccc2F)[C@@H]2CCN(c3cnc4c(-c5ccc6c(c5Cl)CC(=O)N6)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloroindolin-2-one | 503.96 |
| 217 | 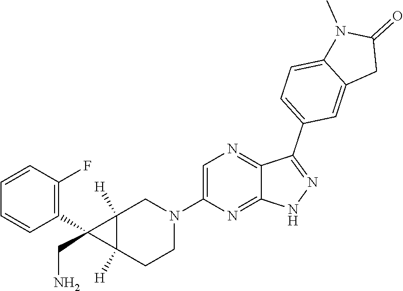 [SMILES: CN1C(=O)Cc2cc(-c3n[nH]c4nc(N5CC[C@@H]6[C@H](C5)[C@@]6(CN)c5ccccc5F)cnc34)ccc21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-methylindolin-2-one | 483.54 |
| 218 | 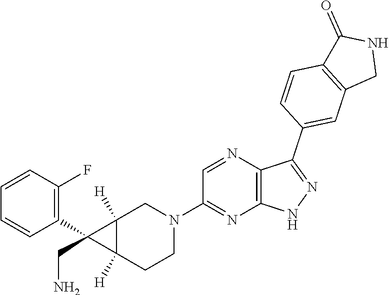 [SMILES: NC[C@]1(c2ccccc2F)[C@@H]2CCN(c3cnc4c(-c5ccc6c(c5)CNC6=O)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)isoindolin-1-one | 469.51 |
| 219 | 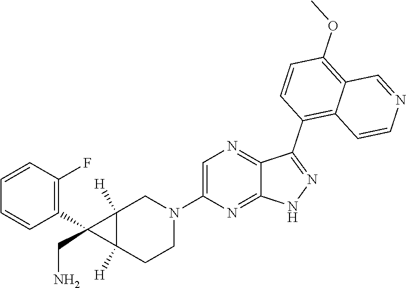 [SMILES: COc1ccc(-c2n[nH]c3nc(N4CC[C@@H]5[C@H](C4)[C@@]5(CN)c4ccccc4F)cnc23)c2ccncc12] | ((1S,6R,7R)-7-(2-fluorophenyl)-3-(3-(8- methoxyisoquinolin-5-yl)-1H-pyrazolo[3,4- b]pyrazin-6-yl)-3-azabicyclo[4.1.0]heptan-7- yl)methanamine | 495.55 |
| 220 | 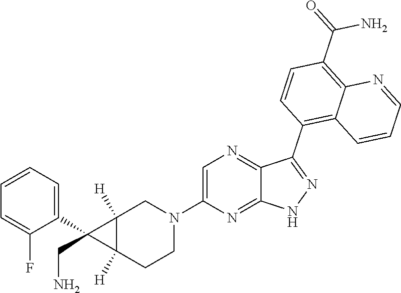 [SMILES: NC[C@]1(c2ccccc2F)[C@@H]2CCN(c3cnc4c(-c5ccc(C(N)=O)c6ncccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinoline-8-carboxamide | 508.55 |
| 221 | 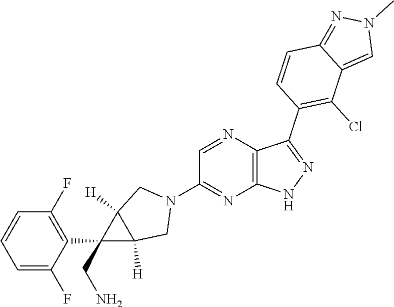 [SMILES: Cn1cc2c(Cl)c(-c3n[nH]c4nc(N5C[C@@H]6[C@H](C5)[C@@]6(CN)c5c(F)cccc5F)cnc34)ccc2n1] | ((1R,5S,6s)-3-(3-(4-chloro-2-methyl-2H-indazol-5- yl)-1H-pyrazolo[3,4-b]pyrazin-6-yl)-6-(2,6- difluorophenyl)-3-azabicyclo[3.1.0]hexan-6- yl)methanamine | 506.94 |
| 222 | 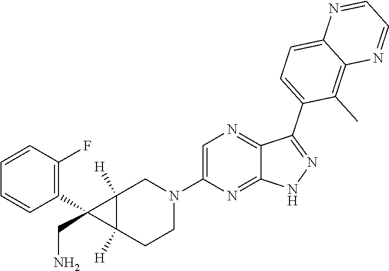 [SMILES: Cc1c(-c2n[nH]c3nc(N4CC[C@@H]5[C@H](C4)[C@@]5(CN)c4ccccc4F)cnc23)ccc2nccnc12] | ((1S,6R,7R)-7-(2-fluorophenyl)-3-(3-(5- methylquinoxalin-6-yl)-1H-pyrazolo[3,4-b]pyrazin-6- yl)-3-azabicyclo[4.1.0]heptan-7-yl)methanamine | 480.54 |
| 223 | 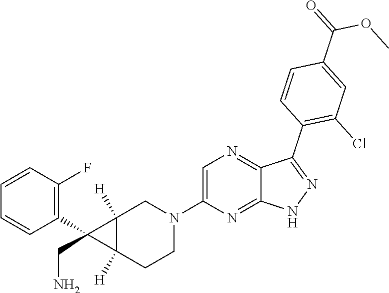 [SMILES: COC(=O)c1ccc(-c2n[nH]c3nc(N4CC[C@@H]5[C@H](C4)[C@@]5(CN)c4ccccc4F)cnc23)c(Cl)c1] | methyl 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorobenzoate | 506.96 |
| 224 | 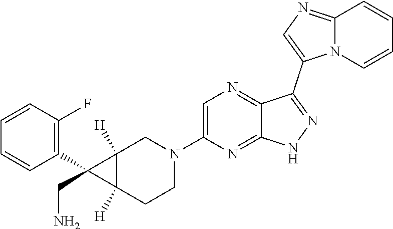 [SMILES: NC[C@]1(c2ccccc2F)[C@@H]2CCN(c3cnc4c(-c5cnc6ccccn56)n[nH]c4n3)C[C@@H]21] | ((1S,6R,7R)-7-(2-fluorophenyl)-3-(3-(imidazo[1,2- a]pyridin-3-yl)-1H-pyrazolo[3,4-b]pyrazin-6-yl)-3- azabicyclo[4.1.0]heptan-7-yl)methanamine | 454.5 |
| 225 | 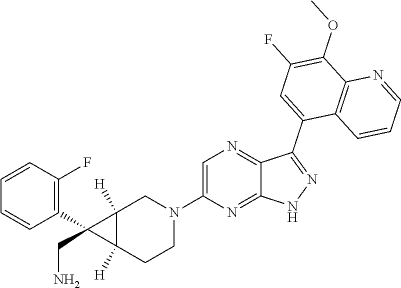 [SMILES: COc1c(F)cc(-c2n[nH]c3nc(N4CC[C@@H]5[C@H](C4)[C@@]5(CN)c4ccccc4F)cnc23)c2cccnc12] | ((1S,6R,7R)-3-(3-(7-fluoro-8-methoxyquinolin-5-yl)- 1H-pyrazolo[3,4-b]pyrazin-6-yl)-7-(2-fluorophenyl)- 3-azabicyclo[4.1.0]heptan-7-yl)methanamine | 513.54 |
| 226 | 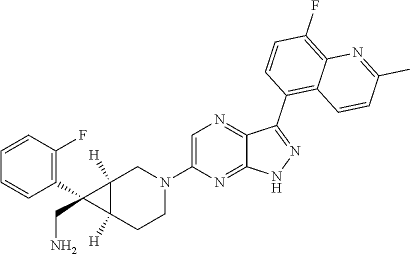 [SMILES: Cc1ccc2c(-c3n[nH]c4nc(N5CC[C@@H]6[C@H](C5)[C@@]6(CN)c5ccccc5F)cnc34)ccc(F)c2n1] | ((1S,6R,7R)-3-(3-(8-fluoro-2-methylquinolin-5-yl)- 1H-pyrazolo[3,4-b]pyrazin-6-yl)-7-(2-fluorophenyl)- 3-azabicyclo[4.1.0]heptan-7-yl)methanamine | 497.54 |
| 227 | 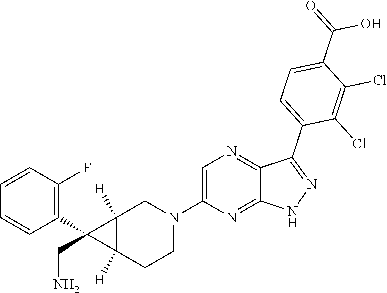 [SMILES: NC[C@]1(c2ccccc2F)[C@@H]2CCN(c3cnc4c(-c5ccc(C(=O)O)c(Cl)c5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,3-dichlorobenzoic acid | 527.38 |
| 228 | 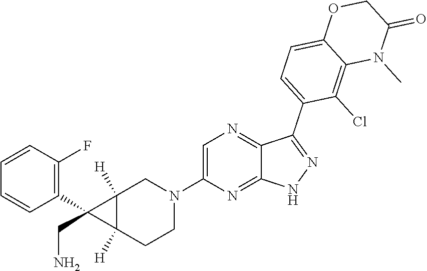 [SMILES: CN1C(=O)COc2ccc(-c3n[nH]c4nc(N5CC[C@@H]6[C@H](C5)[C@@]6(CN)c5ccccc5F)cnc34)c(Cl)c21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-chloro-4-methyl-2H- benzo[b][1,4]oxazin-3(4H)-one | 533.99 |
| 229 | 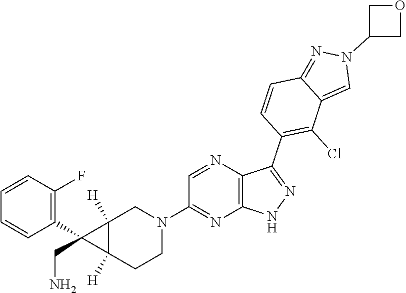 [SMILES: NC[C@]1(c2ccccc2F)[C@@H]2CCN(c3cnc4c(-c5ccc6nn(C7COC7)cc6c5Cl)n[nH]c4n3)C[C@@H]21] | ((1S,6R,7R)-3-(3-(4-chloro-2-(oxetan-3-yl)-2H- indazol-5-yl)-1H-pyrazolo[3,4-b]pyrazin-6-yl)-7-(2- fluorophenyl)-3-azabicyclo[4.1.0]heptan-7- yl)methanamine | 545.01 |
| 230 | 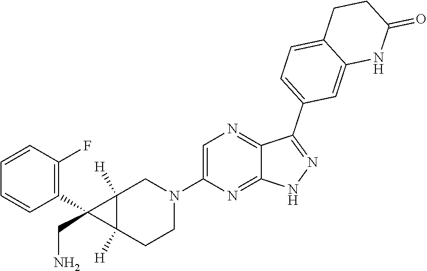 [SMILES: NC[C@]1(c2ccccc2F)[C@@H]2CCN(c3cnc4c(-c5ccc6c(c5)NC(=O)CC6)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroquinolin- 2(1H)-one | 483.54 |
| 231 | 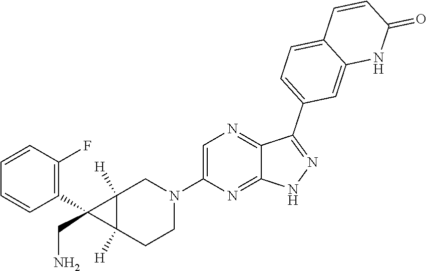 [SMILES: NC[C@]1(c2ccccc2F)[C@@H]2CCN(c3cnc4c(-c5ccc6ccc(=O)[nH]c6c5)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-2(1H)-one | 481.53 |
| 232 | 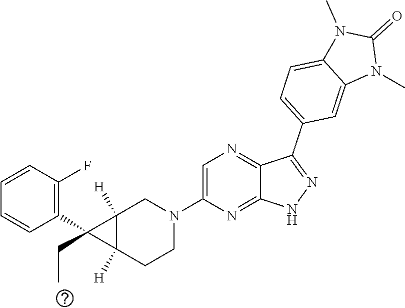 [SMILES: CC[C@]1(c2ccccc2F)[C@@H]2CCN(c3cnc4c(-c5ccc6c(c5)n(C)c(=O)n6C)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1,3-dimethyl-1,3- dihydro-2H-benzo[d]imidazol-2-one | 498.56 |
| 233 | 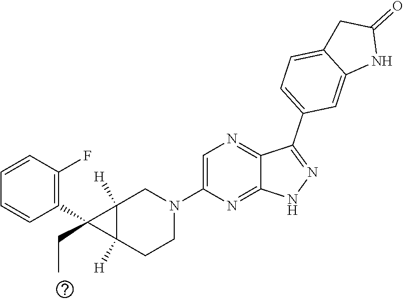 [SMILES: CC[C@]1(c2ccccc2F)[C@@H]2CCN(c3cnc4c(-c5ccc6c(c5)NC(=O)C6)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)indolin-2-one | 469.51 |
| 234 | 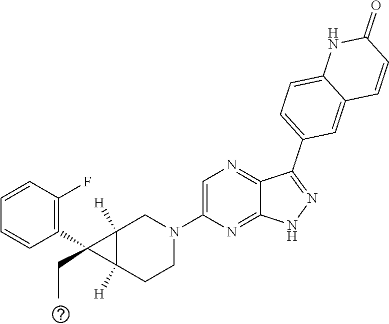 [SMILES: CC[C@]1(c2ccccc2F)[C@@H]2CCN(c3cnc4c(-c5ccc6[nH]c(=O)ccc6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-2(1H)-one | 481.53 |
| 235 | 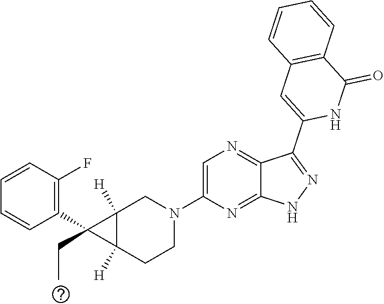 [SMILES: CC[C@]1(c2ccccc2F)[C@@H]2CCN(c3cnc4c(-c5cc6ccccc6c(=O)[nH]5)n[nH]c4n3)C[C@@H]21] | 3-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)isoquinolin-1(2H)-one | 481.53 |
| 236 | 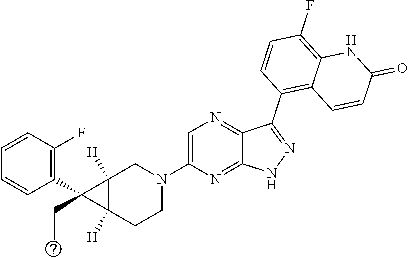 [SMILES: CC[C@]1(c2ccccc2F)[C@@H]2CCN(c3cnc4c(-c5ccc(F)c6[nH]c(=O)ccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-8-fluoroquinolin-2(1H)- one | 499.52 |
| 237 | 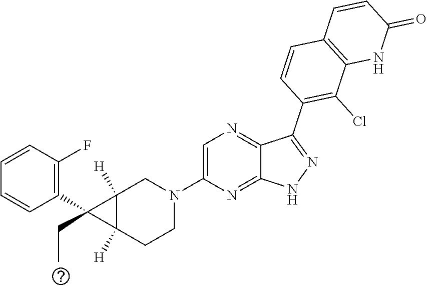 [SMILES: CC[C@]1(c2ccccc2F)[C@@H]2CCN(c3cnc4c(-c5ccc6ccc(=O)[nH]c6c5Cl)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-8-chloroquinolin-2(1H)- one | 515.97 |
| 238 | 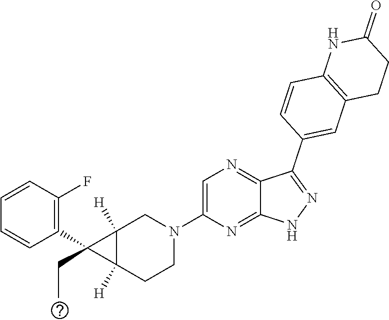 [SMILES: CC[C@]1(c2ccccc2F)[C@@H]2CCN(c3cnc4c(-c5ccc6c(c5)CCC(=O)N6)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroquinolin- 2(1H)-one | 483.54 |
| 239 | 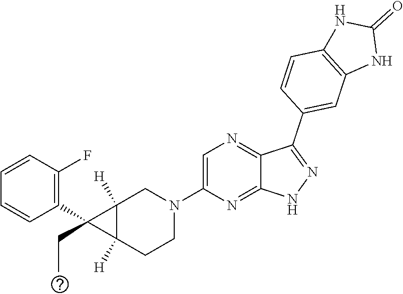 [SMILES: CC[C@]1(c2ccccc2F)[C@@H]2CCN(c3cnc4c(-c5ccc6[nH]c(=O)[nH]c6c5)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1,3-dihydro-2H- benzo[d]imidazol-2-one | 470.5 |
| 240 | 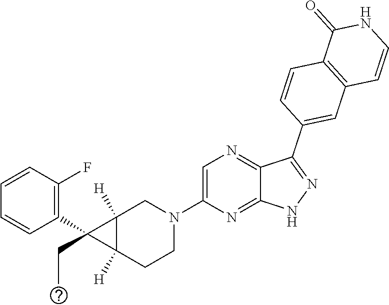 [SMILES: CC[C@]1(c2ccccc2F)[C@@H]2CCN(c3cnc4c(-c5ccc6c(=O)[nH]ccc6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)isoquinolin-1(2H)-one | 481.53 |
| 241 | 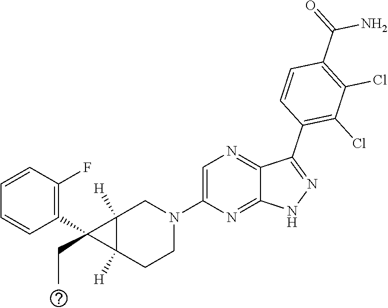 [SMILES: CC[C@]1(c2ccccc2F)[C@@H]2CCN(c3cnc4c(-c5ccc(C(N)=O)c(Cl)c5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,3-dichlorobenzamide | 526.39 |
| 242 | 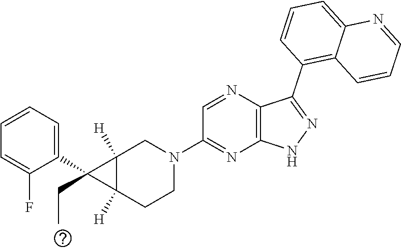 [SMILES: CC[C@]1(c2ccccc2F)[C@@H]2CCN(c3cnc4c(-c5cccc6ncccc56)n[nH]c4n3)C[C@@H]21] | ((1S,6R,7R)-7-(2-fluorophenyl)-3-(3-(quinolin-5-yl)- 1H-pyrazolo[3,4-b]pyrazin-6-yl)-3- azabicyclo[4.1.0]heptan-7-yl)methanamine | 465.53 |
| 243 | 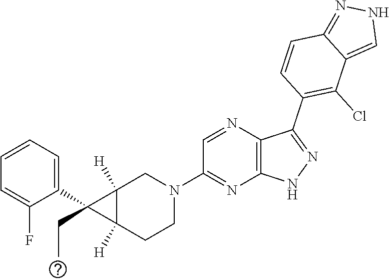 [SMILES: CC[C@]1(c2ccccc2F)[C@@H]2CCN(c3cnc4c(-c5ccc6n[nH]cc6c5Cl)n[nH]c4n3)C[C@@H]21] | ((1S,6R,7R)-3-(3-(4-chloro-2H-indazol-5-yl)-1H- pyrazolo[3,4-b]pyrazin-6-yl)-7-(2-fluorophenyl)-3- azabicyclo[4.1.0]heptan-7-yl)methanamine | 488.95 |
| 244 | 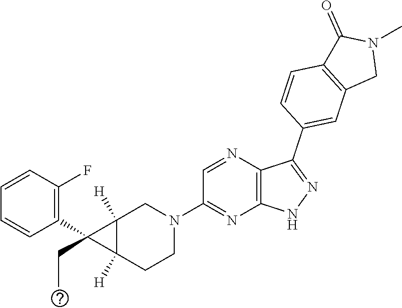 [SMILES: CC[C@]1(c2ccccc2F)[C@@H]2CCN(c3cnc4c(-c5ccc6c(c5)CN(C)C6=O)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2-methylisoindolin-1- one | 483.54 |
| 245 | 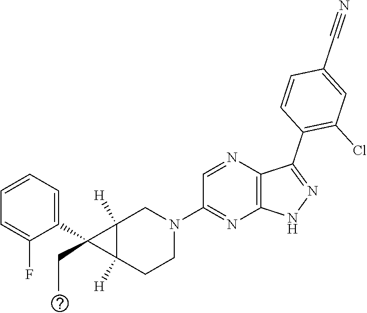 [SMILES: CC[C@]1(c2ccccc2F)[C@@H]2CCN(c3cnc4c(-c5ccc(C#N)cc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorobenzonitrile | 473.93 |
| 246 | 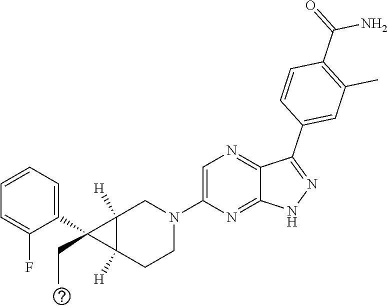 [SMILES: CC[C@]1(c2ccccc2F)[C@@H]2CCN(c3cnc4c(-c5ccc(C(N)=O)c(C)c5)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2-methylbenzamide | 471.53 |
| 247 | 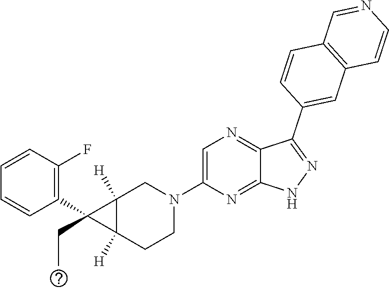 [SMILES: CC[C@]1(c2ccccc2F)[C@@H]2CCN(c3cnc4c(-c5ccc6cnccc6c5)n[nH]c4n3)C[C@@H]21] | ((1S,6R,7R)-7-(2-fluorophenyl)-3-(3-(isoquinolin-6- yl)-1H-pyrazolo[3,4-b]pyrazin-6-yl)-3- azabicyclo[4.1.0]heptan-7-yl)methanamine | 465.53 |
| 248 | 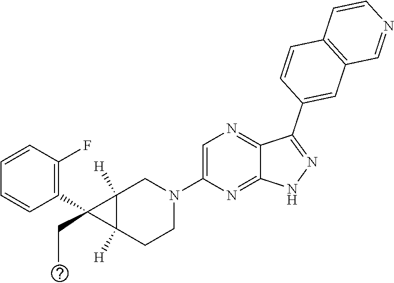 [SMILES: CC[C@]1(c2ccccc2F)[C@@H]2CCN(c3cnc4c(-c5ccc6ccncc6c5)n[nH]c4n3)C[C@@H]21] | ((1S,6R,7R)-7-(2-fluorophenyl)-3-(3-(isoquinolin-7- yl)-1H-pyrazolo[3,4-b]pyrazin-6-yl)-3- azabicyclo[4.1.0]heptan-7-yl)methanamine | 465.53 |
| 249 | 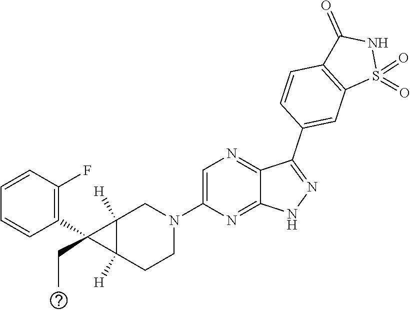 [SMILES: CC[C@]1(c2ccccc2F)[C@@H]2CCN(c3cnc4c(-c5ccc6c(c5)S(=O)(=O)NC6=O)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)benzo[d]isothiazol- 3(2H)-one 1,1-dioxide | 519.55 |
| 250 | 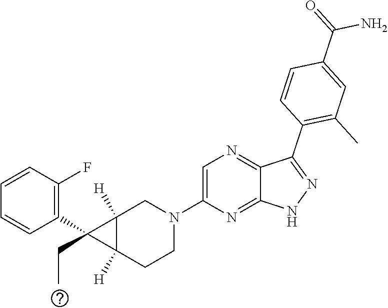 [SMILES: CC[C@]1(c2ccccc2F)[C@@H]2CCN(c3cnc4c(-c5ccc(C(N)=O)cc5C)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-methylbenzamide | 471.53 |
| 251 | 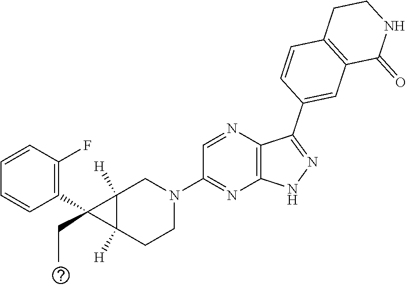 [SMILES: CC[C@]1(c2ccccc2F)[C@@H]2CCN(c3cnc4c(-c5ccc6c(c5)C(=O)NCC6)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroisoquinolin- 1(2H)-one | 483.54 |
| 252 | 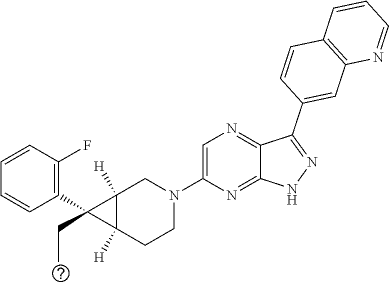 [SMILES: CC[C@]1(c2ccccc2F)[C@@H]2CCN(c3cnc4c(-c5ccc6cccnc6c5)n[nH]c4n3)C[C@@H]21] | ((1S,6R,7R)-7-(2-fluorophenyl)-3-(3-(quinolin-7-yl)- 1H-pyrazolo[3,4-b]pyrazin-6-yl)-3- azabicyclo[4.1.0]heptan-7-yl)methanamine | 465.53 |
| 253 | 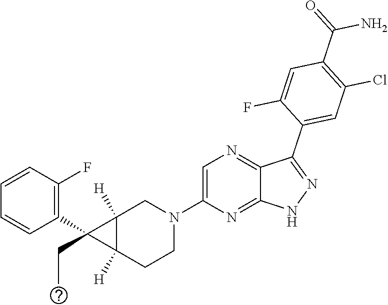 [SMILES: CC[C@]1(c2ccccc2F)[C@@H]2CCN(c3cnc4c(-c5cc(Cl)c(C(N)=O)cc5F)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2-chloro-5- fluorobenzamide | 509.94 |
| 254 | 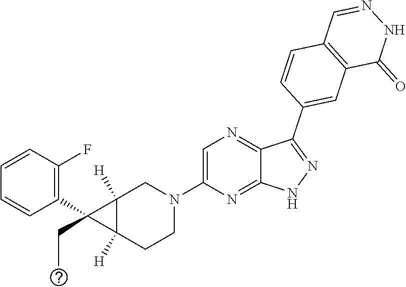 [SMILES: CC[C@]1(c2ccccc2F)[C@@H]2CCN(c3cnc4c(-c5ccc6cn[nH]c(=O)c6c5)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)phthalazin-1(2H)-one | 482.51 |
| 255 | 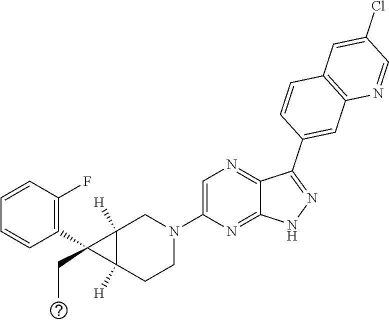 [SMILES: CC[C@]1(c2ccccc2F)[C@@H]2CCN(c3cnc4c(-c5ccc6cc(Cl)cnc6c5)n[nH]c4n3)C[C@@H]21] | ((1S,6R,7R)-3-(3-(3-chloroquinolin-7-yl)-1H- pyrazolo[3,4-b]pyrazin-6-yl)-7-(2-fluorophenyl)-3- azabicyclo[4.1.0]heptan-7-yl)methanamine | 499.97 |
| 256 | 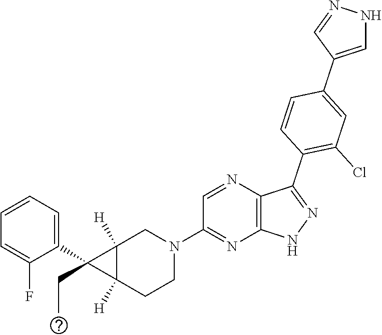 [SMILES: CC[C@]1(c2ccccc2F)[C@@H]2CCN(c3cnc4c(-c5ccc(-c6cn[nH]c6)cc5Cl)n[nH]c4n3)C[C@@H]21] | ((1S,6R,7R)-3-(3-(2-chloro-4-(1H-pyrazol-4- yl)phenyl)-1H-pyrazolo[3,4-b]pyrazin-6-yl)-7-(2- fluorophenyl)-3-azabicyclo[4.1.0]heptan-7- yl)methanamine | 514.99 |
| 257 | 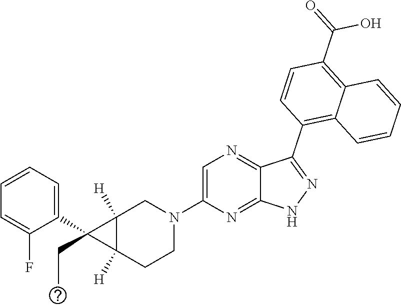 [SMILES: CC[C@]1(c2ccccc2F)[C@@H]2CCN(c3cnc4c(-c5ccc(C(=O)O)c6ccccc56)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-naphthoic acid | 508.55 |
| 258 | 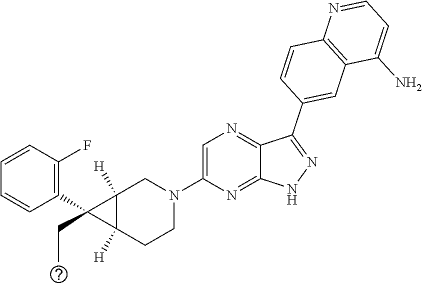 [SMILES: CC[C@]1(c2ccccc2F)[C@@H]2CCN(c3cnc4c(-c5ccc6nccc(N)c6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinolin-4-amine | 480.54 |
| 259 | 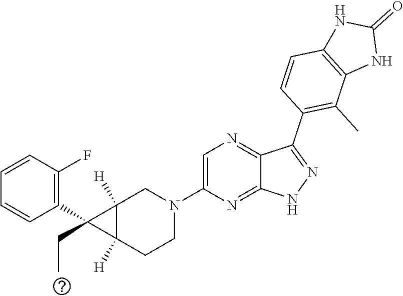 [SMILES: CC[C@]1(c2ccccc2F)[C@@H]2CCN(c3cnc4c(-c5ccc6[nH]c(=O)[nH]c6c5C)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-methyl-1,3-dihydro- 2H-benzo[d]imidazol-2-one | 484.53 |
| 260 | 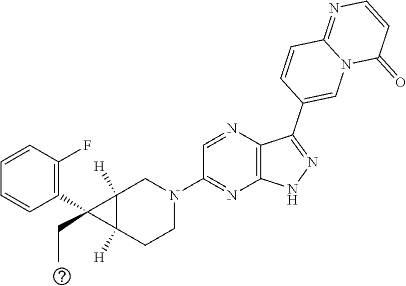 [SMILES: CC[C@]1(c2ccccc2F)[C@@H]2CCN(c3cnc4c(-c5ccc6nccc(=O)n6c5)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4H-pyrido[1,2- a]pyrimidin-4-one | 482.51 |
| 261 | 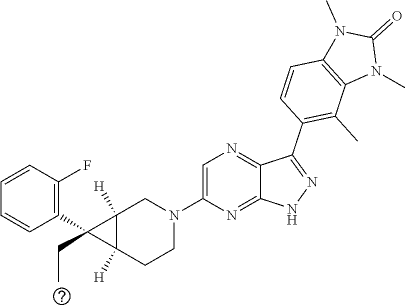 [SMILES: CC[C@]1(c2ccccc2F)[C@@H]2CCN(c3cnc4c(-c5ccc6c(c5C)n(C)c(=O)n6C)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1,3,4-trimethyl-1,3- dihydro-2H-benzo[d]imidazol-2-one | 512.58 |
| 262 | 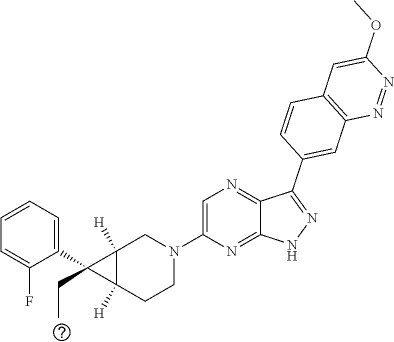 [SMILES: CC[C@]1(c2ccccc2F)[C@@H]2CCN(c3cnc4c(-c5ccc6cc(OC)nnc6c5)n[nH]c4n3)C[C@@H]21] | ((1S,6R,7R)-7-(2-fluorophenyl)-3-(3-(3- methoxycinnolin-7-yl)-1H-pyrazolo[3,4-b]pyrazin-6- yl)-3-azabicyclo[4.1.0]heptan-7-yl)methanamine | 496.54 |
| 263 | 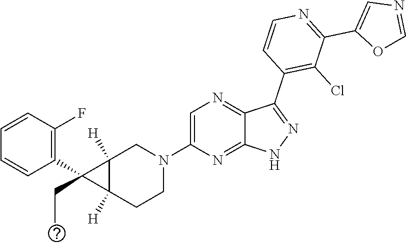 [SMILES: CC[C@]1(c2ccccc2F)[C@@H]2CCN(c3cnc4c(-c5ccnc(-c6cnco6)c5Cl)n[nH]c4n3)C[C@@H]21] | ((1S,6R,7R)-3-(3-(3-chloro-2-(oxazol-5-yl)pyridin-4- yl)-1H-pyrazolo[3,4-b]pyrazin-6-yl)-7-(2- fluorophenyl)-3-azabicyclo[4.1.0]heptan-7- yl)methanamine | 516.96 |
| 264 | 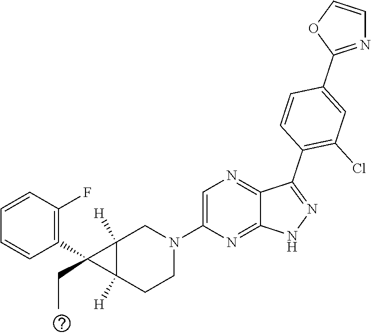 [SMILES: CC[C@]1(c2ccccc2F)[C@@H]2CCN(c3cnc4c(-c5ccc(-c6ncco6)cc5Cl)n[nH]c4n3)C[C@@H]21] | ((1S,6R,7R)-3-(3-(2-chloro-4-(oxazol-2-yl)phenyl)- 1H-pyrazolo[3,4-b]pyrazin-6-yl)-7-(2-fluorophenyl)- 3-azabicyclo[4.1.0]heptan-7-yl)methanamine | 515.97 |
| 265 | 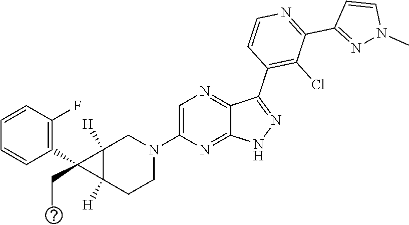 [SMILES: CC[C@]1(c2ccccc2F)[C@@H]2CCN(c3cnc4c(-c5ccnc(-c6ccn(C)n6)c5Cl)n[nH]c4n3)C[C@@H]21] | ((1S,6R,7R)-3-(3-(3-chloro-2-(1-methyl-1H-pyrazol- 3-yl)pyridin-4-yl)-1H-pyrazolo[3,4-b]pyrazin-6-yl)- 7-(2-fluorophenyl)-3-azabicyclo[4.1.0]heptan-7- yl)methanamine | 530 |
| 266 | 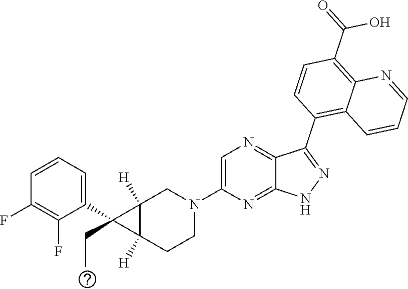 [SMILES: CC[C@]1(c2cccc(F)c2F)[C@@H]2CCN(c3cnc4c(-c5ccc(C(=O)O)c6ncccc56)n[nH]c4n3)C[C@@H]21] | 5-(6-((1S,6R,7R)-7-(aminomethyl)-7-(2,3- difluorophenyl)-3-azabicyclo[4.1.0]heptan-3-yl)-1H- pyrazolo[3,4-b]pyrazin-3-yl)quinoline-8-carboxylic acid | 527.53 |
| 267 | 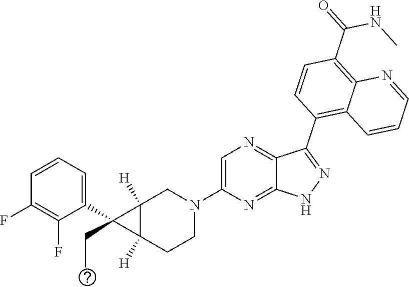 [SMILES: CC[C@]1(c2cccc(F)c2F)[C@@H]2CCN(c3cnc4c(-c5ccc(C(=O)NC)c6ncccc56)n[nH]c4n3)C[C@@H]21] | 5-(6-((1S,6R,7R)-7-(aminomethyl)-7-(2,3- difluorophenyl)-3-azabicyclo[4.1.0]heptan-3-yl)-1H- pyrazolo[3,4-b]pyrazin-3-yl)-N-methylquinoline-8- carboxamide | 540.57 |
| 268 | 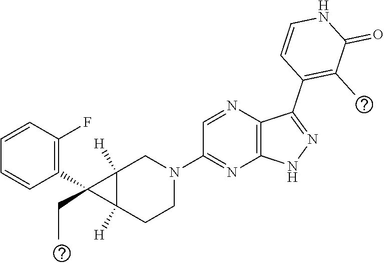 [SMILES: CC[C@]1(c2ccccc2F)[C@@H]2CCN(c3cnc4c(-c5cc[nH]c(=O)c5C)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloropyridin-2(1H)- one | 465.91 |
| 269 | 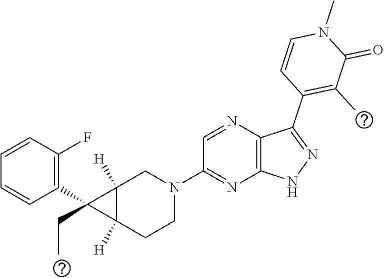 [SMILES: CC[C@]1(c2ccccc2F)[C@@H]2CCN(c3cnc4c(-c5ccn(C)c(=O)c5C)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-1- methylpyridin-2(1H)-one | 479.94 |
| 270 | 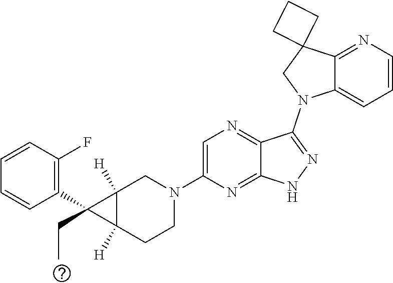 [SMILES: CC[C@]1(c2ccccc2F)[C@@H]2CCN(c3cnc4c(N5CC6(CCC6)c6ncccc65)n[nH]c4n3)C[C@@H]21] | ((1S,6R,7R)-7-(2-fluorophenyl)-3-(3- (spiro[cyclobutane-1,3′-pyrrolo[3,2-b]pyridin]- 1′(2′H)-yl)-1H-pyrazolo[3,4-b]pyrazin-6-yl)-3- azabicyclo[4.1.0]heptan-7-yl)methanamine | 496.58 |
| 271 | 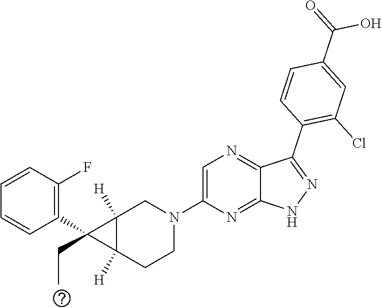 [SMILES: CC[C@]1(c2ccccc2F)[C@@H]2CCN(c3cnc4c(-c5ccc(C(=O)O)cc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorobenzoic acid | 492.93 |
| 272 | 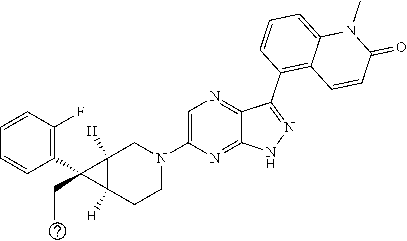 [SMILES: CC[C@]1(c2ccccc2F)[C@@H]2CCN(c3cnc4c(-c5cccc6c5ccc(=O)n6C)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-methylquinolin- 2(1H)-one | 495.55 |
| 273 | 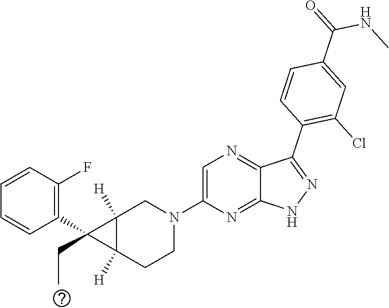 [SMILES: CC[C@]1(c2ccccc2F)[C@@H]2CCN(c3cnc4c(-c5ccc(C(=O)NC)cc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-N- methylbenzamide | 505.98 |
| 274 | 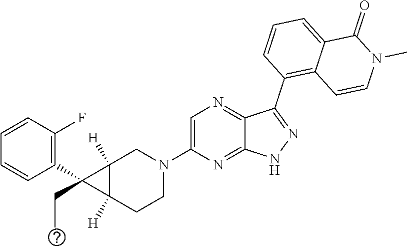 [SMILES: CC[C@]1(c2ccccc2F)[C@@H]2CCN(c3cnc4c(-c5cccc6c(=O)n(C)ccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2-methylisoquinolin- 1(2H)-one | 495.55 |
| 275 | 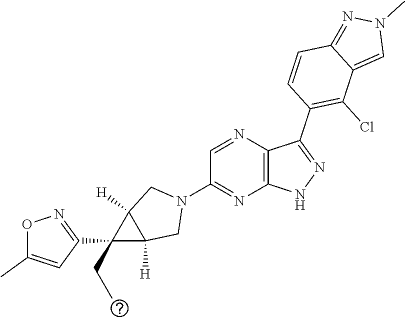 | ((1R,5S,6r)-3-(3-(4-chloro-2-methyl-2H-indazol-5- yl)-1H-pyrazolo[3,4-b]pyrazin-6-yl)-6-(5- methylisoxazol-3-yl)-3-azabicyclo[3.1.0]hexan-6- yl)methanamine | 475.93 |
| 276 | 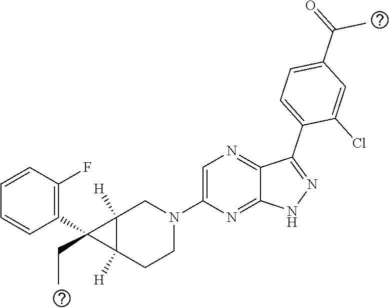 [SMILES: CC[C@]1(c2ccccc2F)[C@@H]2CCN(c3cnc4c(-c5ccc(C(C)=O)cc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorobenzamide | 491.95 |
| 277 | 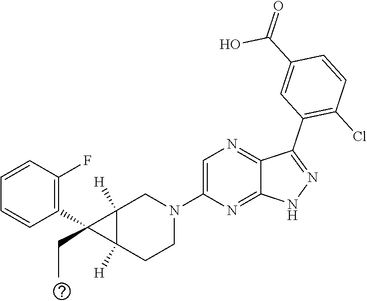 [SMILES: CC[C@]1(c2ccccc2F)[C@@H]2CCN(c3cnc4c(-c5cc(C(=O)O)ccc5Cl)n[nH]c4n3)C[C@@H]21] | 3-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chlorobenzoic acid | 492.93 |
| 278 | 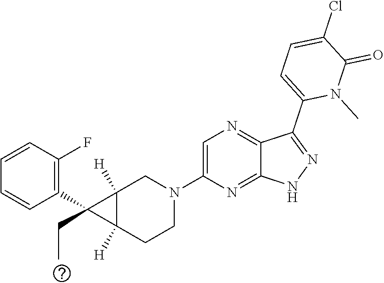 [SMILES: CC[C@]1(c2ccccc2F)[C@@H]2CCN(c3cnc4c(-c5ccc(Cl)c(=O)n5C)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-1- methylpyridin-2(1H)-one | 479.94 |
| 279 | 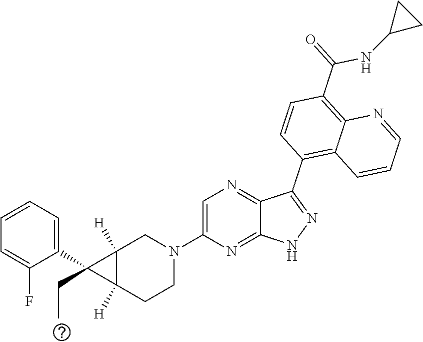 [SMILES: CC[C@]1(c2ccccc2F)[C@@H]2CCN(c3cnc4c(-c5ccc(C(=O)NC6CC6)c6ncccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-N- cyclopropylquinoline-8-carboxamide | 548.61 |
| 280 | 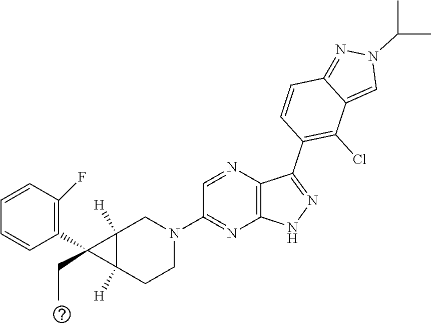 [SMILES: CC[C@]1(c2ccccc2F)[C@@H]2CCN(c3cnc4c(-c5ccc6nn(C(C)C)cc6c5Cl)n[nH]c4n3)C[C@@H]21] | ((1S,6R,7R)-3-(3-(4-chloro-2-isopropyl-2H-indazol-5- yl)-1H-pyrazolo[3,4-b]pyrazin-6-yl)-7-(2- fluorophenyl)-3-azabicyclo[4.1.0]heptan-7- yl)methanamine | 531.03 |
| 281 | 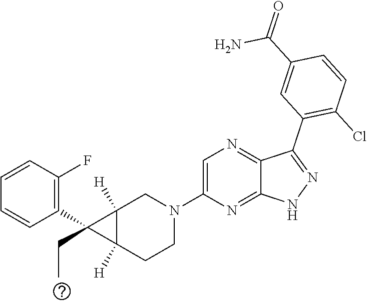 [SMILES: CC[C@]1(c2ccccc2F)[C@@H]2CCN(c3cnc4c(-c5cc(C(N)=O)ccc5Cl)n[nH]c4n3)C[C@@H]21] | 3-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chlorobenzamide | 491.95 |
| 282 | 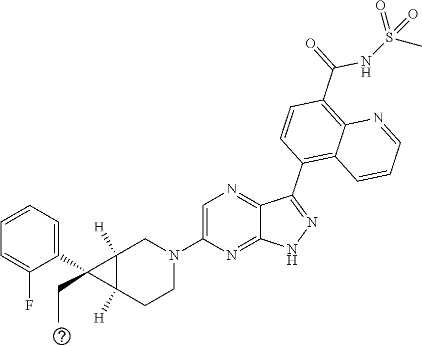 [SMILES: CC[C@]1(c2ccccc2F)[C@@H]2CCN(c3cnc4c(-c5ccc(C(=O)NS(C)(=O)=O)c6ncccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-N- (methylsulfonyl)quinoline-8-carboxamide | 586.64 |
| 283 | 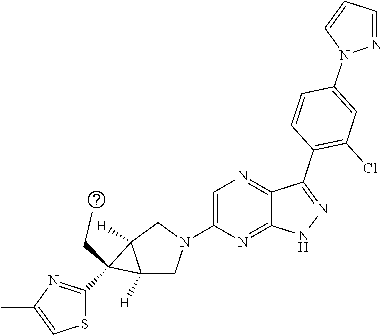 [SMILES: CC[C@]1(c2nc(C)cs2)[C@@H]2CN(c3cnc4c(-c5ccc(-n6cccn6)cc5Cl)n[nH]c4n3)C[C@@H]21] | ((1R,5S,6r)-3-(3-(2-chloro-4-(1H-pyrazol-1- yl)phenyl)-1H-pyrazolo[3,4-b]pyrazin-6-yl)-6-(4- methylthiazol-2-yl)-3-azabicyclo[3.1.0]hexan-6- yl)methanamine | 504.01 |
| 284 | 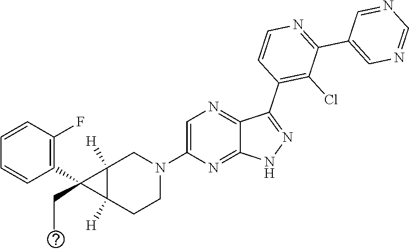 [SMILES: CC[C@]1(c2ccccc2F)[C@@H]2CCN(c3cnc4c(-c5ccnc(-c6cncnc6)c5Cl)n[nH]c4n3)C[C@@H]21] | ((1S,6R,7R)-3-(3-(3-chloro-2-(pyrimidin-5- yl)pyridin-4-yl)-1H-pyrazolo[3,4-b]pyrazin-6-yl)-7- (2-fluorophenyl)-3-azabicyclo[4.1.0]heptan-7- yl)methanamine | 527.98 |
| 285 | 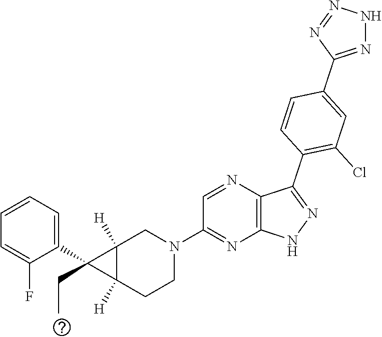 [SMILES: CC[C@]1(c2ccccc2F)[C@@H]2CCN(c3cnc4c(-c5ccc(-c6nn[nH]n6)cc5Cl)n[nH]c4n3)C[C@@H]21] | ((1S,6R,7R)-3-(3-(2-chloro-4-(2H-tetrazol-5- yl)phenyl)-1H-pyrazolo[3,4-b]pyrazin-6-yl)-7-(2- fluorophenyl)-3-azabicyclo[4.1.0]heptan-7- yl)methanamine | 516.96 |
| 286 | 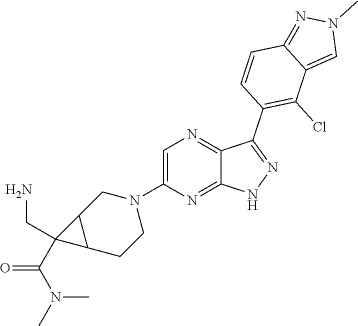 [SMILES: CN(C)C(=O)C1(CN)C2CCN(c3cnc4c(-c5ccc6nn(C)cc6c5Cl)n[nH]c4n3)CC21] | 7-(aminomethyl)-3-(3-(4-chloro-2-methyl-2H- indazol-5-yl)-1H-pyrazolo[3,4-b]pyrazin-6-yl)-N,N- dimethyl-3-azabicyclo[4.1.0]heptane-7-carboxamide | 479.97 |
| 287 | 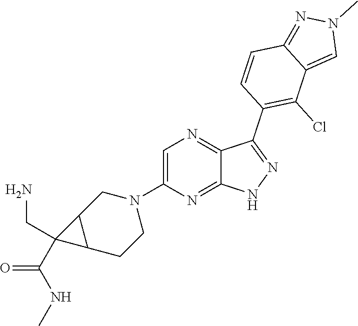 [SMILES: CNC(=O)C1(CN)C2CCN(c3cnc4c(-c5ccc6nn(C)cc6c5Cl)n[nH]c4n3)CC21] | 7-(aminomethyl)-3-(3-(4-chloro-2-methyl-2H- indazol-5-yl)-1H-pyrazolo[3,4-b]pyrazin-6-yl)-N- methyl-3-azabicyclo[4.1.0]heptane-7-carboxamide | 465.94 |
| 288 | 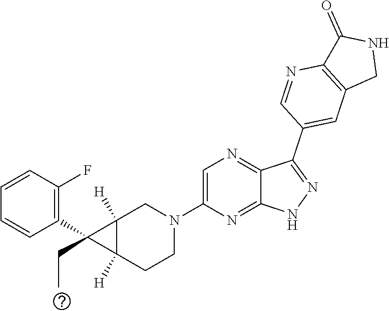 [SMILES: CC[C@]1(c2ccccc2F)[C@@H]2CCN(c3cnc4c(-c5cnc6c(c5)CNC6=O)n[nH]c4n3)C[C@@H]21] | 3-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1,4,5,6-tetrahydro-7H- pyrrolo[3,4-b]pyridin-7-one | 470.5 |
| 289 | 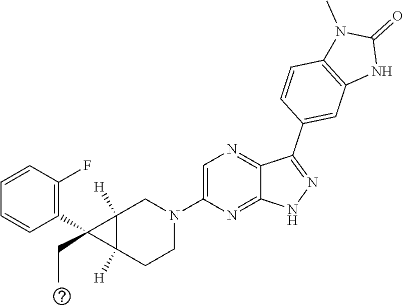 [SMILES: CC[C@]1(c2ccccc2F)[C@@H]2CCN(c3cnc4c(-c5ccc6c(c5)[nH]c(=O)n6C)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-methyl-1,3-dihydro- 2H-benzo[d]imidazol-2-one | 484.53 |
| 290 | 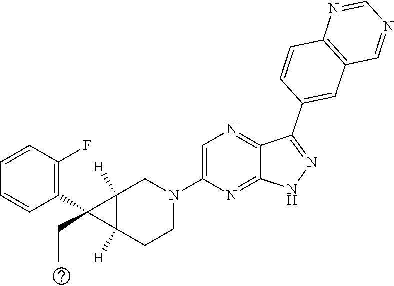 [SMILES: CC[C@]1(c2ccccc2F)[C@@H]2CCN(c3cnc4c(-c5ccc6ncncc6c5)n[nH]c4n3)C[C@@H]21] | ((1S,6R,7R)-7-(2-fluorophenyl)-3-(3-(quinazolin-6- yl)-1H-pyrazolo[3,4-b]pyrazin-6-yl)-3- azabicyclo[4.1.0]heptan-7-yl)methanamine | 466.51 |
| 291 | 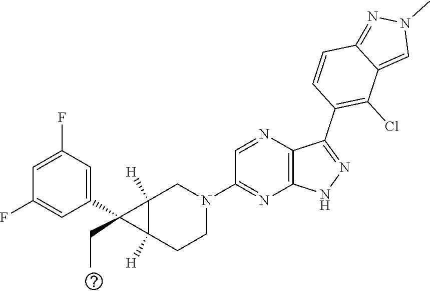 [SMILES: CC[C@]1(c2cc(F)cc(F)c2)[C@@H]2CCN(c3cnc4c(-c5ccc6nn(C)cc6c5Cl)n[nH]c4n3)C[C@@H]21] | ((1S,6R,7R)-3-(3-(4-chloro-2-methyl-2H-indazol-5- yl)-1H-pyrazolo[3,4-b]pyrazin-6-yl)-7-(3,5- difluorophenyl)-3-azabicyclo[4.1.0]heptan-7- yl)methanamine | 520.96 |
| 292 | 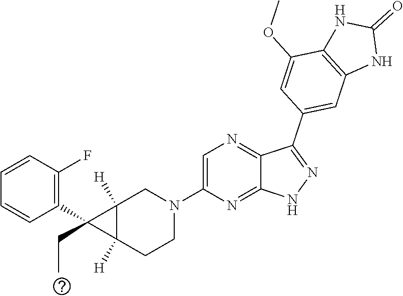 [SMILES: CC[C@]1(c2ccccc2F)[C@@H]2CCN(c3cnc4c(-c5cc(OC)c6[nH]c(=O)[nH]c6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-methoxy-1,3-dihydro- 2H-benzo[d]imidazol-2-one | 500.53 |
| 293 | 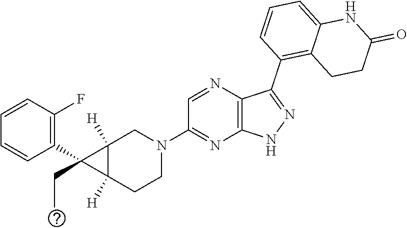 [SMILES: CC[C@]1(c2ccccc2F)[C@@H]2CCN(c3cnc4c(-c5cccc6c5CCC(=O)N6)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroquinolin- 2(1H)-one | 483.54 |
| 294 | 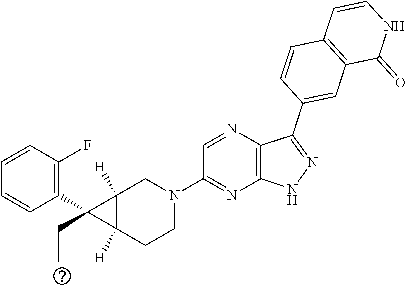 [SMILES: CC[C@]1(c2ccccc2F)[C@@H]2CCN(c3cnc4c(-c5ccc6cc[nH]c(=O)c6c5)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)isoquinolin-1(2H)-one | 481.52 |
| 295 | 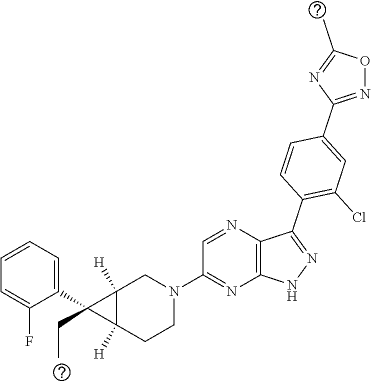 [SMILES: CC[C@]1(c2ccccc2F)[C@@H]2CCN(c3cnc4c(-c5ccc(-c6noc(C)n6)cc5Cl)n[nH]c4n3)C[C@@H]21] | 3-(4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorophenyl)-1,2,4- oxadiazol-5-ol | 532.96 |
| 296 | 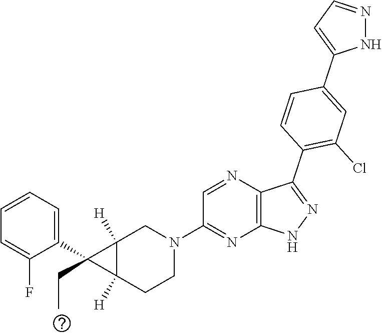 [SMILES: CC[C@]1(c2ccccc2F)[C@@H]2CCN(c3cnc4c(-c5ccc(-c6ccn[nH]6)cc5Cl)n[nH]c4n3)C[C@@H]21] | ((1S,6R,7R)-3-(3-(2-chloro-4-(1H-pyrazol-5- yl)phenyl)-1H-pyrazolo[3,4-b]pyrazin-6-yl)-7-(2- fluorophenyl)-3-azabicyclo[4.1.0]heptan-7- yl)methanamine | 514.98 |
| 297 | 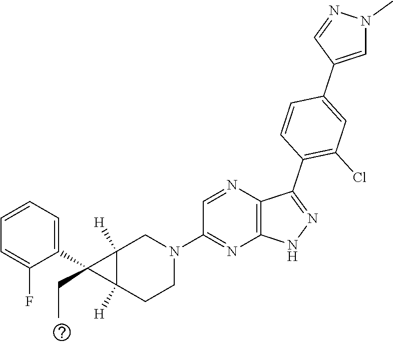 [SMILES: CC[C@]1(c2ccccc2F)[C@@H]2CCN(c3cnc4c(-c5ccc(-c6cnn(C)c6)cc5Cl)n[nH]c4n3)C[C@@H]21] | ((1S,6R,7R)-3-(3-(2-chloro-4-(1-methyl-1H-pyrazol- 4-yl)phenyl)-1H-pyrazolo[3,4-b]pyrazin-6-yl)-7-(2- fluorophenyl)-3-azabicyclo[4.1.0]heptan-7- yl)methanamine | 529.01 |
| 298 | 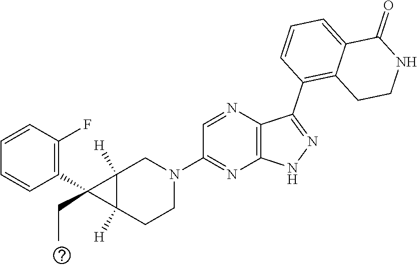 [SMILES: CC[C@]1(c2ccccc2F)[C@@H]2CCN(c3cnc4c(-c5cccc6c5CCNC6=O)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroisoquinolin- 1(2H)-one | 483.54 |
| 299 | 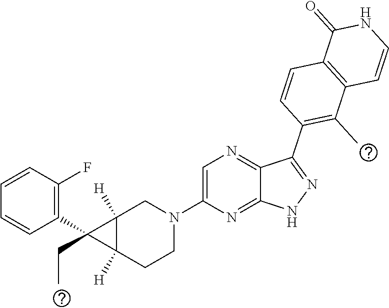 [SMILES: CC[C@]1(c2ccccc2F)[C@@H]2CCN(c3cnc4c(-c5ccc6c(=O)[nH]ccc6c5C)n[nH]c4n3)C[C@@H]21] | 6-(6-((1S,6R,7R)-7-(aminomethyl)-7-(2- fluorophenyl)-3-azabicyclo[4.1.0]heptan-3-yl)-4H- 114-pyrazolo[4,5-b]pyrazin-3-yl)-5-fluoroisoquinolin- 1(2H)-one | 499.51 |
| 300 | 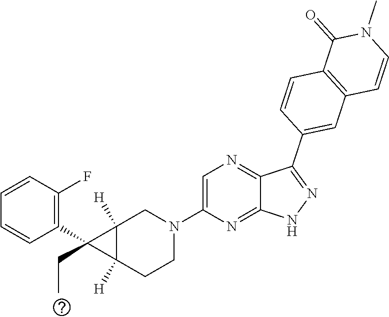 [SMILES: CC[C@]1(c2ccccc2F)[C@@H]2CCN(c3cnc4c(-c5ccc6c(=O)n(C)ccc6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)- 114,212-pyrazolo[3,4-b]pyrazin-3-yl)-2- methylisoquinolin-1(2H)-one | 495.55 |
| 301 | 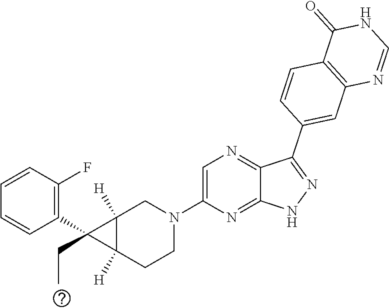 [SMILES: CC[C@]1(c2ccccc2F)[C@@H]2CCN(c3cnc4c(-c5ccc6c(=O)[nH]cnc6c5)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)quinazolin-4(3H)-one | 482.51 |
| 302 | 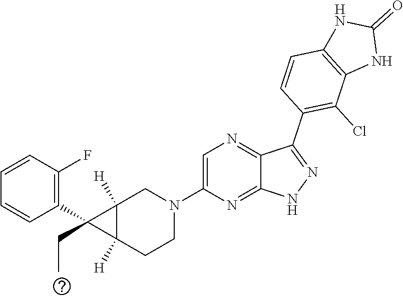 [SMILES: CC[C@]1(c2ccccc2F)[C@@H]2CCN(c3cnc4c(-c5ccc6[nH]c(=O)[nH]c6c5Cl)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-1,3-dihydro- 2H-benzo[d]imidazol-2-one | 504.95 |
| 303 | 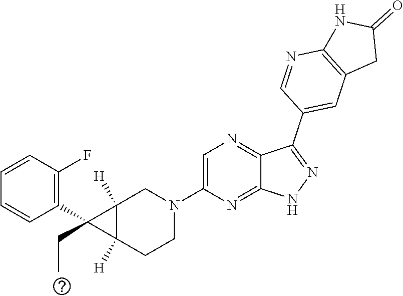 [SMILES: CC[C@]1(c2ccccc2F)[C@@H]2CCN(c3cnc4c(-c5cnc6c(c5)CC(=O)N6)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1,3-dihydro-2H- pyrrolo[2,3-b]pyridin-2-one | 470.5 |
| 304 | 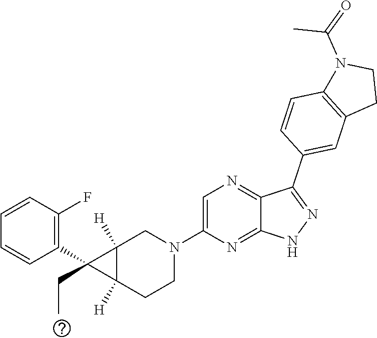 [SMILES: CC[C@]1(c2ccccc2F)[C@@H]2CCN(c3cnc4c(-c5ccc6c(c5)CCN6C(C)=O)n[nH]c4n3)C[C@@H]21] | 1-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)indolin-1-yl)ethan-1-one | 497.57 |
| 305 | 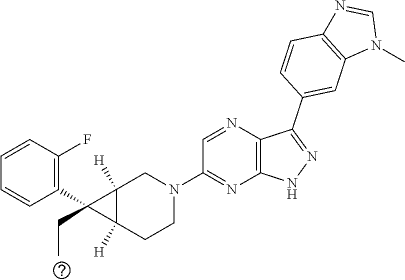 [SMILES: CC[C@]1(c2ccccc2F)[C@@H]2CCN(c3cnc4c(-c5ccc6ncn(C)c6c5)n[nH]c4n3)C[C@@H]21] | ((1S,6R,7R)-7-(2-fluorophenyl)-3-(3-(1-methyl-1H- benzo[d]imidazol-6-yl)-1H-pyrazolo[3,4-b]pyrazin-6- yl)-3-azabicyclo[4.1.0]heptan-7-yl)methanamine | 468.53 |
| 306 | 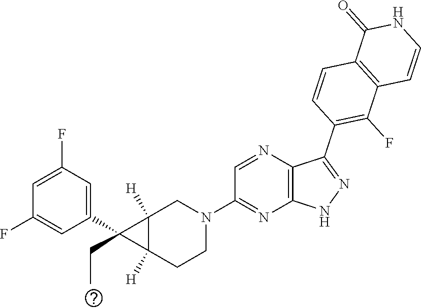 [SMILES: CC[C@]1(c2cc(F)cc(F)c2)[C@@H]2CCN(c3cnc4c(-c5ccc6c(=O)[nH]ccc6c5F)n[nH]c4n3)C[C@@H]21] | 6-(6-((1S,6R,7R)-7-(aminomethyl)-7-(3,5- difluorophenyl)-3-azabicyclo[4.1.0]heptan-3-yl)-1H- pyrazolo[3,4-b]pyrazin-3-yl)-5-fluoroisoquinolin- 1(2H)-one | 517.51 |
| 307 | 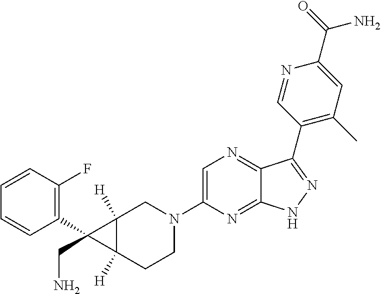 [SMILES: Cc1cc(C(N)=O)ncc1-c1n[nH]c2nc(N3CC[C@@H]4[C@H](C3)[C@@]4(CN)c3ccccc3F)cnc12] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-methylpicolinamide | 472.52 |
| 308 | 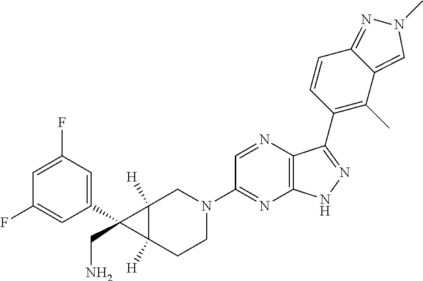 [SMILES: Cc1c(-c2n[nH]c3nc(N4CC[C@@H]5[C@H](C4)[C@@]5(CN)c4cc(F)cc(F)c4)cnc23)ccc2nn(C)cc12] | ((1S,6R,7R)-7-(3,5-difluorophenyl)-3-(3-(2,4- dimethyl-2H-indazol-5-yl)-1H-pyrazolo[3,4- b]pyrazin-6-yl)-3-azabicyclo[4.1.0]heptan-7- yl)methanamine | 500.55 |
| 309 | 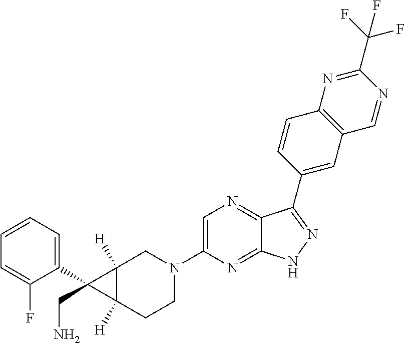 [SMILES: NC[C@]1(c2ccccc2F)[C@@H]2CCN(c3cnc4c(-c5ccc6nc(C(F)(F)F)ncc6c5)n[nH]c4n3)C[C@@H]21] | ((1S,6R,7R)-7-(2-fluorophenyl)-3-(3-(2- (trifluoromethyl)quinazolin-6-yl)-1H-pyrazolo[3,4- b]pyrazin-6-yl)-3-azabicyclo[4.1.0]heptan-7- yl)methanamine | 534.51 |
| 310 | 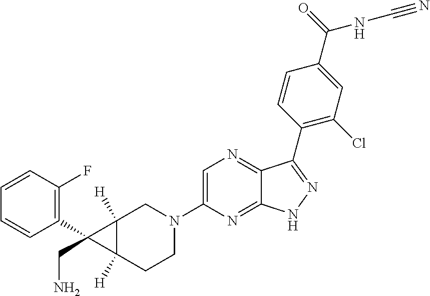 [SMILES: N#CNC(=O)c1ccc(-c2n[nH]c3nc(N4CC[C@@H]5[C@H](C4)[C@@]5(CN)c4ccccc4F)cnc23)c(Cl)c1] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-N- cyanobenzamide | 516.96 |
| 311 | 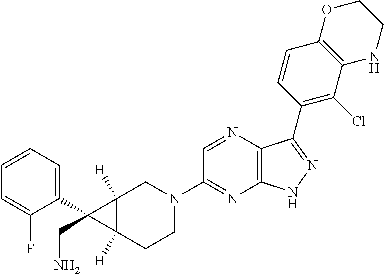 [SMILES: NC[C@]1(c2ccccc2F)[C@@H]2CCN(c3cnc4c(-c5ccc6c(c5Cl)NCCO6)n[nH]c4n3)C[C@@H]21] | ((1S,6R,7R)-3-(3-(5-chloro-3,4-dihydro-2H- benzo[b][1,4]oxazin-6-yl)-1,7-dihydro-412- pyrazolo[4,3-b]pyrazin-6-yl)-7-(2-fluorophenyl)-3- azabicyclo[4.1.0]heptan-7-yl)methanamine | 505.97 |
| 312 | 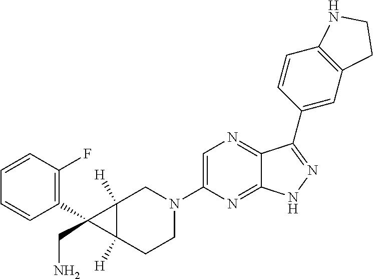 [SMILES: NC[C@]1(c2ccccc2F)[C@@H]2CCN(c3cnc4c(-c5ccc6c(c5)CCN6)n[nH]c4n3)C[C@@H]21] | ((1S,6R,7R)-7-(2-fluorophenyl)-3-(3-(indolin-5-yl)- 1H-pyrazolo[3,4-b]pyrazin-6-yl)-3- azabicyclo[4.1.0]heptan-7-yl)methanamine | 455.53 |
| 313 | 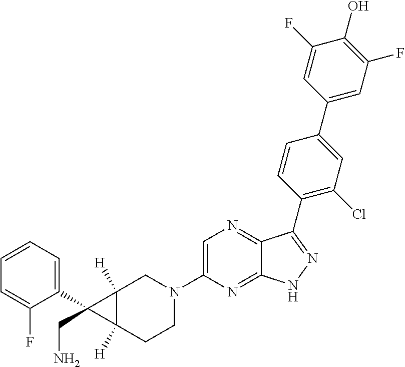 [SMILES: NC[C@]1(c2ccccc2F)[C@@H]2CCN(c3cnc4c(-c5ccc(-c6cc(F)c(O)c(F)c6)cc5Cl)n[nH]c4n3)C[C@@H]21] | 4′-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3′-chloro-3,5-difluoro- 2,3,4,5-tetrahydro-[1,1′-biphenyl]-4-ol | 577 |
| 314 | 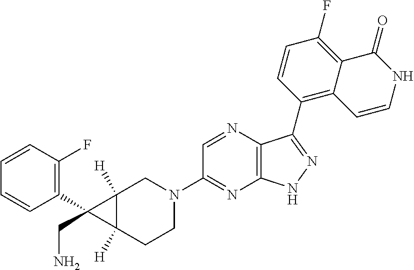 [SMILES: NC[C@]1(c2ccccc2F)[C@@H]2CCN(c3cnc4c(-c5ccc(F)c6c(=O)[nH]ccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-4,7- dihydro-1H-pyrazolo[3,4-b]pyrazin-3-yl)-8- fluoroisoquinolin-1(2H)-one | 499.51 |
| 315 | 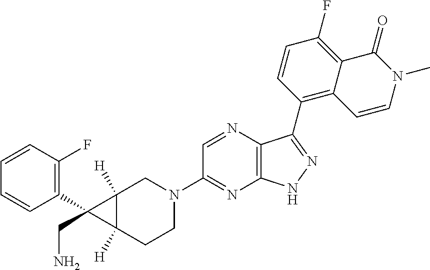 [SMILES: Cn1ccc2c(-c3n[nH]c4nc(N5CC[C@@H]6[C@H](C5)[C@@]6(CN)c5ccccc5F)cnc34)ccc(F)c2c1=O] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-8-fluoro-2- methylisoquinolin-1(2H)-one | 513.54 |
| 316 | 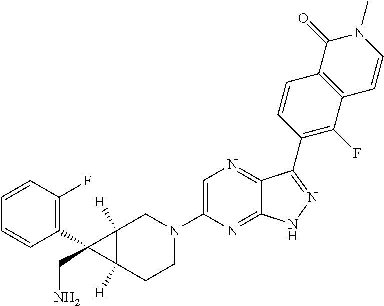 [SMILES: Cn1ccc2c(F)c(-c3n[nH]c4nc(N5CC[C@@H]6[C@H](C5)[C@@]6(CN)c5ccccc5F)cnc34)ccc2c1=O] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-fluoro-2- methylisoquinolin-1(2H)-one | 513.54 |
| 317 | 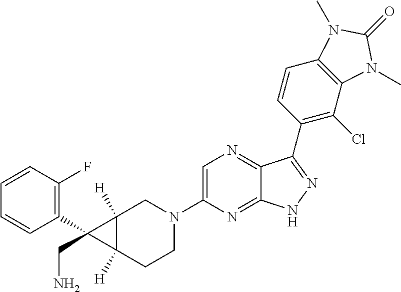 [SMILES: Cn1c(=O)n(C)c2c(Cl)c(-c3n[nH]c4nc(N5CC[C@@H]6[C@H](C5)[C@@]6(CN)c5ccccc5F)cnc34)ccc21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-1,3-dimethyl- 1,3-dihydro-2H-benzo[d]imidazol-2-one | 533 |
| 318 | 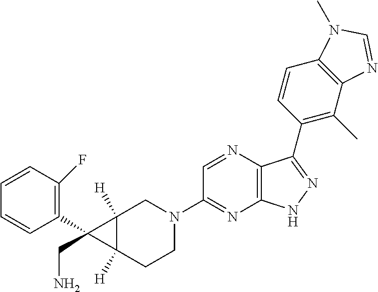 [SMILES: Cc1c(-c2n[nH]c3nc(N4CC[C@@H]5[C@H](C4)[C@@]5(CN)c4ccccc4F)cnc23)ccc2c1ncn2C] | ((1S,6R,7R)-3-(3-(1,4-dimethyl-4,7-dihydro-1H- benzo[d]imidazol-5-yl)-1H-pyrazolo[3,4-b]pyrazin-6- yl)-7-(2-fluorophenyl)-3-azabicyclo[4.1.0]heptan-7- yl)methanamine | 482.56 |
| 319 | 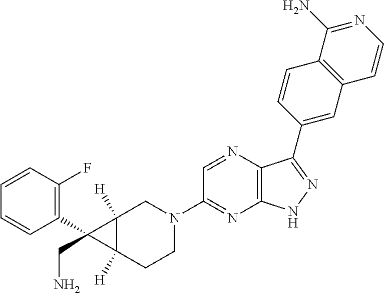 [SMILES: NC[C@]1(c2ccccc2F)[C@@H]2CCN(c3cnc4c(-c5ccc6c(N)nccc6c5)n[nH]c4n3)C[C@@H]21] | 6-(6-((1S,6R,7R)-7-(aminomethyl)-7-(2- fluorophenyl)-3-azabicyclo[4.1.0]heptan-3-yl)-2,4- dihydro-1H-pyrazolo[3,4-b]pyrazin-3-yl)isoquinolin- 1-amine | 480.54 |
| 320 | 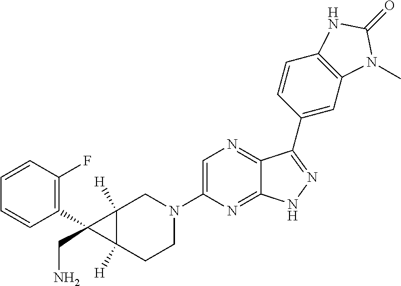 [SMILES: Cn1c(=O)[nH]c2ccc(-c3n[nH]c4nc(N5CC[C@@H]6[C@H](C5)[C@@]6(CN)c5ccccc5F)cnc34)cc21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-1-methyl-1,3-dihydro- 2H-benzo[d]imidazol-2-one | 484.53 |
| 321 | 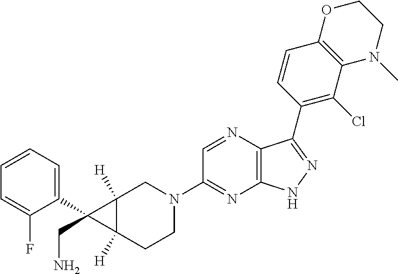 [SMILES: CN1CCOc2ccc(-c3n[nH]c4nc(N5CC[C@@H]6[C@H](C5)[C@@]6(CN)c5ccccc5F)cnc34)c(Cl)c21] | ((1S,6R,7R)-3-(3-(5-chloro-4-methyl-3,4-dihydro- 2H-benzo[b][1,4]oxazin-6-yl)-2,3,4,7-tetrahydro-1H- pyrazolo[3,4-b]pyrazin-6-yl)-7-(2-fluorophenyl)-3- azabicyclo[4.1.0]heptan-7-yl)methanamine | 520 |
| 322 | 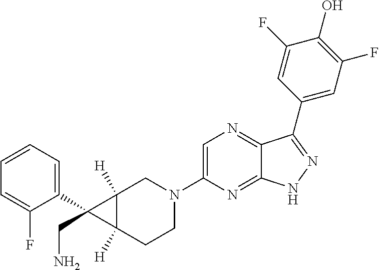 [SMILES: NC[C@]1(c2ccccc2F)[C@@H]2CCN(c3cnc4c(-c5cc(F)c(O)c(F)c5)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,6-difluorophenol | 466.46 |
| 323 | 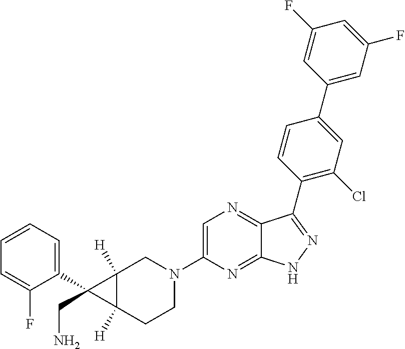 [SMILES: NC[C@]1(c2ccccc2F)[C@@H]2CCN(c3cnc4c(-c5ccc(-c6cc(F)cc(F)c6)cc5Cl)n[nH]c4n3)C[C@@H]21] | ((1S,6R,7R)-3-(3-(3-chloro-3′,5′-difluoro-[1,1′- biphenyl]-4-yl)-1H-pyrazolo[3,4-b]pyrazin-6-yl)-7- (2-fluorophenyl)-3-azabicyclo[4.1.0]heptan-7- yl)methanamine | 561 |
| 324 | 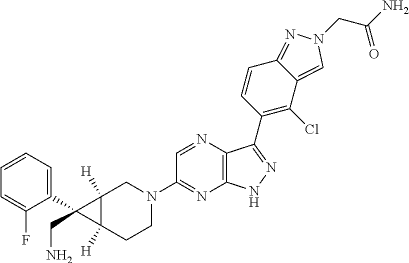 [SMILES: NC[C@]1(c2ccccc2F)[C@@H]2CCN(c3cnc4c(-c5ccc6nn(CC(N)=O)cc6c5Cl)n[nH]c4n3)C[C@@H]21] | 2-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-2H-indazol-2- yl)acetamide | 546 |
| 325 | 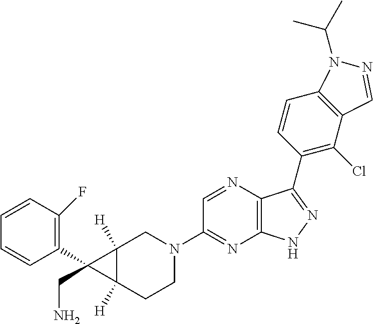 [SMILES: CC(C)n1ncc2c(Cl)c(-c3n[nH]c4nc(N5CC[C@@H]6[C@H](C5)[C@@]6(CN)c5ccccc5F)cnc34)ccc21] | ((1S,6R,7R)-3-(3-(4-chloro-1-isopropyl-1H-indazol-5- yl)-1H-pyrazolo[3,4-b]pyrazin-6-yl)-7-(2- fluorophenyl)-3-azabicyclo[4.1.0]heptan-7- yl)methanamine | 531.03 |
| 326 | 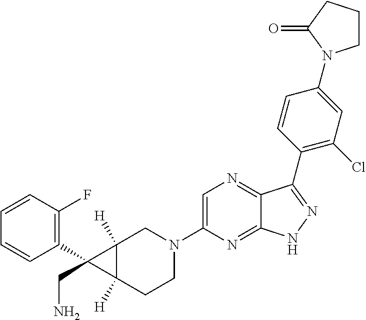 [SMILES: NC[C@]1(c2ccccc2F)[C@@H]2CCN(c3cnc4c(-c5ccc(N6CCCC6=O)cc5Cl)n[nH]c4n3)C[C@@H]21] | 1-(4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3- chlorophenyl)pyrrolidin-2-one | 532.01 |
| 327 | 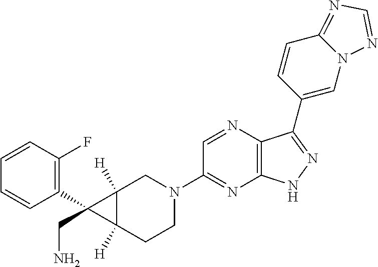 [SMILES: NC[C@]1(c2ccccc2F)[C@@H]2CCN(c3cnc4c(-c5ccc6ncnn6c5)n[nH]c4n3)C[C@@H]21] | ((1S,6R,7R)-3-(3-([1,2,4]triazolo[1,5-a]pyridin-6-yl)- 1H-pyrazolo[3,4-b]pyrazin-6-yl)-7-(2-fluorophenyl)- 3-azabicyclo[4.1.0]heptan-7-yl)methanamine | 455.49 |
| 328 | 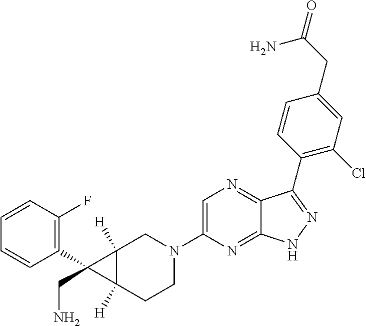 [SMILES: NC[C@]1(c2ccccc2F)[C@@H]2CCN(c3cnc4c(-c5ccc(CC(N)=O)cc5Cl)n[nH]c4n3)C[C@@H]21] | 2-(4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3- chlorophenyl)acetamide | 505.97 |
| 329 | 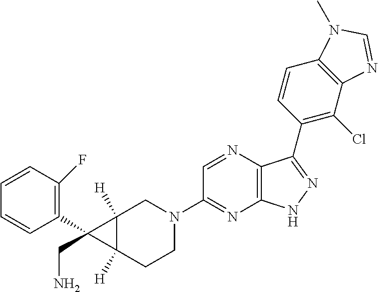 [SMILES: Cn1cnc2c(Cl)c(-c3n[nH]c4nc(N5CC[C@@H]6[C@H](C5)[C@@]6(CN)c5ccccc5F)cnc34)ccc21] | ((1S,6R,7R)-3-(3-(4-chloro-1-methyl-1H- benzo[d]imidazol-5-yl)-1H-pyrazolo[3,4-b]pyrazin-6- yl)-7-(2-fluorophenyl)-3-azabicyclo[4.1.0]heptan-7- yl)methanamine | 502.97 |
| 330 | 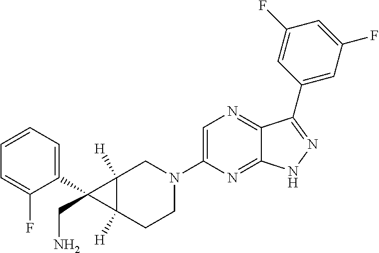 [SMILES: NC[C@]1(c2ccccc2F)[C@@H]2CCN(c3cnc4c(-c5cc(F)cc(F)c5)n[nH]c4n3)C[C@@H]21] | ((1S,6R,7R)-3-(3-(3,5-difluorophenyl)-1H- pyrazolo[3,4-b]pyrazin-6-yl)-7-(2-fluorophenyl)-3- azabicyclo[4.1.0]heptan-7-yl)methanamine | 450.46 |
| 331 | 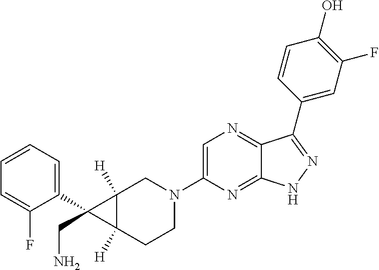 [SMILES: NC[C@]1(c2ccccc2F)[C@@H]2CCN(c3cnc4c(-c5ccc(O)c(F)c5)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2-fluorophenol | 448.47 |
| 332 | 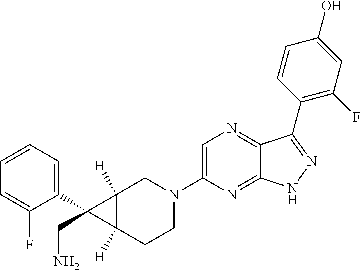 [SMILES: NC[C@]1(c2ccccc2F)[C@@H]2CCN(c3cnc4c(-c5ccc(O)cc5F)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-fluorophenol | 448.47 |
| 333 | 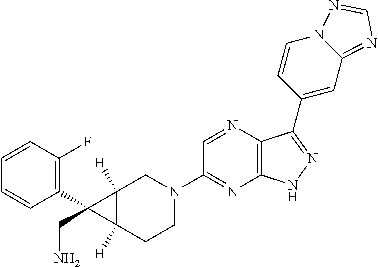 [SMILES: NC[C@]1(c2ccccc2F)[C@@H]2CCN(c3cnc4c(-c5ccn6ncnc6c5)n[nH]c4n3)C[C@@H]21] | ((1S,6R,7R)-3-(3-([1,2,4]triazolo[1,5-a]pyridin-7-yl)- 1H-pyrazolo[3,4-b]pyrazin-6-yl)-7-(2-fluorophenyl)- 3-azabicyclo[4.1.0]heptan-7-yl)methanamine | 455.49 |
| 334 | 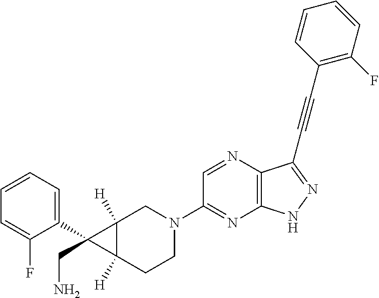 [SMILES: NC[C@]1(c2ccccc2F)[C@@H]2CCN(c3cnc4c(C#Cc5ccccc5F)n[nH]c4n3)C[C@@H]21] | ((1S,6R,7R)-7-(2-fluorophenyl)-3-(3-((2- fluorophenyl)ethynyl)-1H-pyrazolo[3,4-b]pyrazin-6- yl)-3-azabicyclo[4.1.0]heptan-7-yl)methanamine | 456.49 |
| 335 | 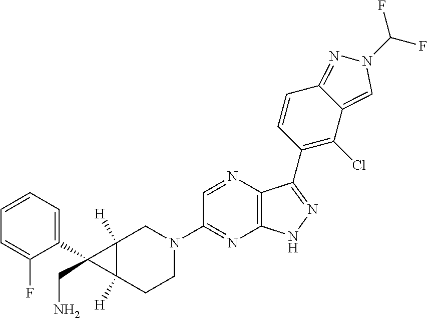 [SMILES: NC[C@]1(c2ccccc2F)[C@@H]2CCN(c3cnc4c(-c5ccc6nn(C(F)F)cc6c5Cl)n[nH]c4n3)C[C@@H]21] | ((1S,6R,7R)-3-(3-(4-chloro-2-(difluoromethyl)-2H- indazol-5-yl)-1H-pyrazolo[3,4-b]pyrazin-6-yl)-7-(2- fluorophenyl)-3-azabicyclo[4.1.0]heptan-7- yl)methanamine | 538.95 |
| 336 | 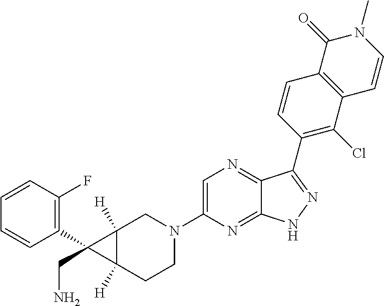 [SMILES: Cn1ccc2c(Cl)c(-c3n[nH]c4nc(N5CC[C@@H]6[C@H](C5)[C@@]6(CN)c5ccccc5F)cnc34)ccc2c1=O] | 6-(6-((1S,6R,7R)-7-(aminomethyl)-7-(2- fluorophenyl)-3-azabicyclo[4.1.0]heptan-3-yl)-7H- 114-pyrazolo[4,5-b]pyrazin-3-yl)-5-chloro-2- methylisoquinolin-1(2H)-one | 530 |
| 337 | 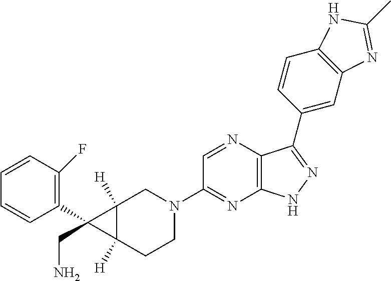 [SMILES: Cc1nc2cc(-c3n[nH]c4nc(N5CC[C@@H]6[C@H](C5)[C@@]6(CN)c5ccccc5F)cnc34)ccc2[nH]1] | ((1S,6R,7R)-7-(2-fluorophenyl)-3-(3-(2-methyl-1H- benzo[d]imidazol-5-yl)-1H-pyrazolo[3,4-b]pyrazin-6- yl)-3-azabicyclo[4.1.0]heptan-7-yl)methanamine | 468.53 |
| 338 | 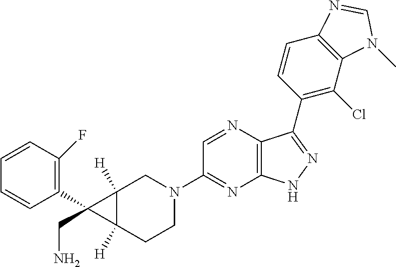 [SMILES: Cn1cnc2ccc(-c3n[nH]c4nc(N5CC[C@@H]6[C@H](C5)[C@@]6(CN)c5ccccc5F)cnc34)c(Cl)c21] | ((1S,6R,7R)-3-(3-(7-chloro-1-methyl-1H- benzo[d]imidazol-6-yl)-1H-pyrazolo[3,4-b]pyrazin-6- yl)-7-(2-fluorophenyl)-3-azabicyclo[4.1.0]heptan-7- yl)methanamine | 502.97 |
| 339 | 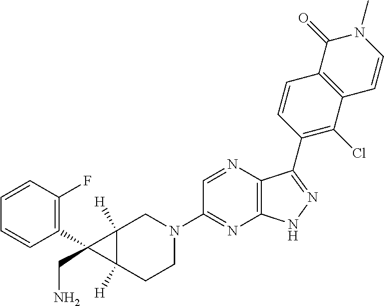 [SMILES: Cn1ccc2c(Cl)c(-c3n[nH]c4nc(N5CC[C@@H]6[C@H](C5)[C@@]6(CN)c5ccccc5F)cnc34)ccc2c1=O] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-chloro-2- methylphthalazin-1(2H)-one | 530.98 |
| 340 | 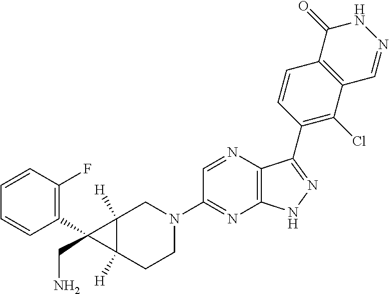 [SMILES: NC[C@]1(c2ccccc2F)[C@@H]2CCN(c3cnc4c(-c5ccc6c(=O)[nH]ncc6c5Cl)n[nH]c4n3)C[C@@H]21] | 6-(6-((1S,6R,7R)-7-(aminomethyl)-7-(2- fluorophenyl)-3-azabicyclo[4.1.0]heptan-3-yl)- 114,212-pyrazolo[3,4-b]pyrazin-3-yl)-5- chlorophthalazin-1(2H)-one | 516.96 |
| 341 | 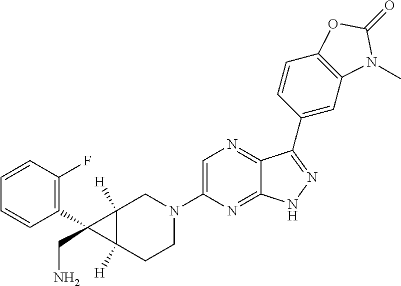 [SMILES: Cn1c(=O)oc2ccc(-c3n[nH]c4nc(N5CC[C@@H]6[C@H](C5)[C@@]6(CN)c5ccccc5F)cnc34)cc21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3- methylbenzo[d]oxazol-2(3H)-one | 485.51 |
| 342 | 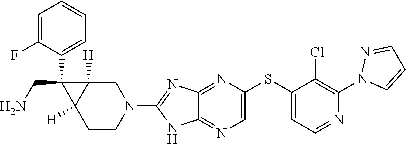 [SMILES: NC[C@]1(c2ccccc2F)[C@@H]2CCN(c3nc4nc(Sc5ccnc(-n6cccn6)c5Cl)cnc4[nH]3)C[C@@H]21] | ((1S,6R,7R)-3-(5-((3-chloro-2-(1H-pyrazol-1- yl)pyridin-4-yl)thio)-1H-imidazo[4,5-b]pyrazin-2-yl)- 7-(2-fluorophenyl)-3-azabicyclo[4.1.0]heptan-7- yl)methanamine | 548.04 |
| 343 | 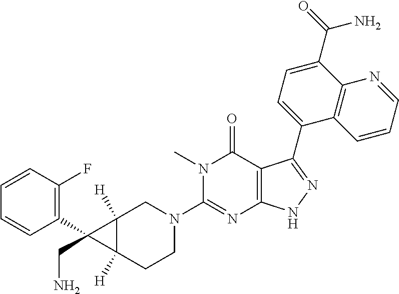 [SMILES: Cn1c(N2CC[C@@H]3[C@H](C2)[C@@]3(CN)c2ccccc2F)nc2[nH]nc(-c3ccc(C(N)=O)c4ncccc34)c2c1=O] | 5-(6-((1S,6R,7R)-7-(aminomethyl)-7-(2- fluorophenyl)-3-azabicyclo[4.1.0]heptan-3-yl)-5- methyl-4-oxo-4,5-dihydro-1H-pyrazolo[3,4- d]pyrimidin-3-yl)quinoline-8-carboxamide | 538.58 |
| 344 | 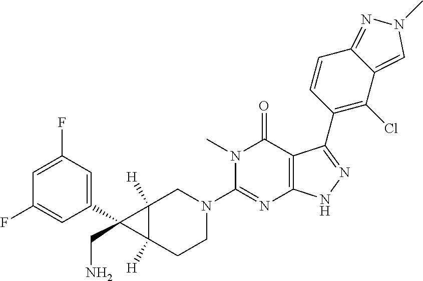 [SMILES: Cn1cc2c(Cl)c(-c3n[nH]c4nc(N5CC[C@@H]6[C@H](C5)[C@@]6(CN)c5cc(F)cc(F)c5)n(C)c(=O)c34)ccc2n1] | 6-((1S,6R,7R)-7-(aminomethyl)-7-(3,5- difluorophenyl)-3-azabicyclo[4.1.0]heptan-3-yl)-3-(4- chloro-2-methyl-2H-indazol-5-yl)-5-methyl-1,5- dihydro-4H-pyrazolo[3,4-d]pyrimidin-4-one | 550.99 |
| 345 | 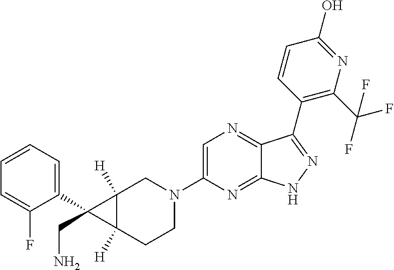 [SMILES: NC[C@]1(c2ccccc2F)[C@@H]2CCN(c3cnc4c(-c5ccc(O)nc5C(F)(F)F)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-6- (trifluoromethyl)pyridin-2-ol | 499,46 |
| 346 | 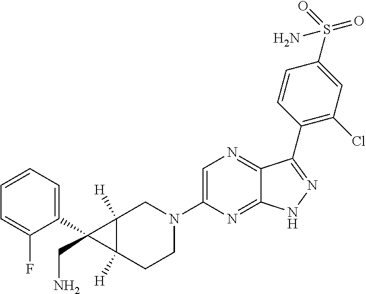 [SMILES: NC[C@]1(c2ccccc2F)[C@@H]2CCN(c3cnc4c(-c5ccc(S(N)(=O)=O)cc5Cl)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3- chlorobenzenesulfonamide | 528 |
| 347 | 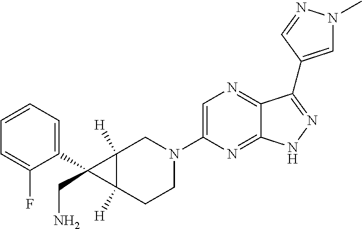 [SMILES: Cn1cc(-c2n[nH]c3nc(N4CC[C@@H]5[C@H](C4)[C@@]5(CN)c4ccccc4F)cnc23)cn1] | ((1S,6R,7R)-7-(2-fluorophenyl)-3-(3-(1-methyl-1H- pyrazol-4-yl)-1H-pyrazolo[3,4-b]pyrazin-6-yl)-3- azabicyclo[4.1.0]heptan-7-yl)methanamine | 418.47 |
| 348 | 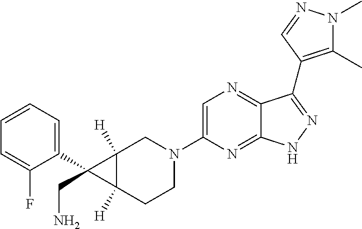 [SMILES: Cc1c(-c2n[nH]c3nc(N4CC[C@@H]5[C@H](C4)[C@@]5(CN)c4ccccc4F)cnc23)cnn1C] | ((1S,6R,7R)-3-(3-(1,5-dimethyl-1H-pyrazol-4-yl)- 1H-pyrazolo[3,4-b]pyrazin-6-yl)-7-(2-fluorophenyl)- 3-azabicyclo[4.1.0]heptan-7-yl)methanamine | 432.5 |
| 349 | 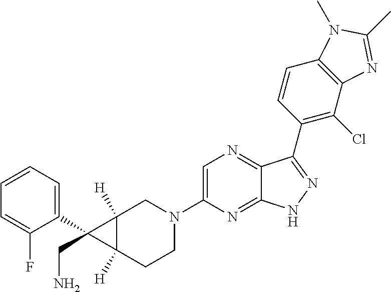 [SMILES: Cc1nc2c(Cl)c(-c3n[nH]c4nc(N5CC[C@@H]6[C@H](C5)[C@@]6(CN)c5ccccc5F)cnc34)ccc2n1C] | ((1S,6R,7R)-3-(3-(4-chloro-1,2-dimethyl-1H- benzo[d]imidazol-5-yl)-1H-pyrazolo[3,4-b]pyrazin-6- yl)-7-(2-fluorophenyl)-3-azabicyclo[4.1.0]heptan-7- yl)methanamine | 517 |
| 350 | 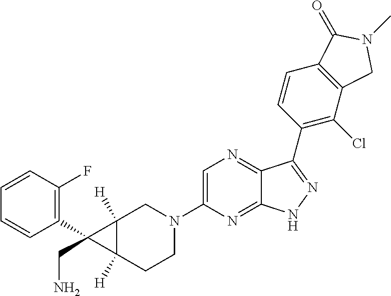 [SMILES: CN1Cc2c(ccc(-c3n[nH]c4nc(N5CC[C@@H]6[C@H](C5)[C@@]6(CN)c5ccccc5F)cnc34)c2Cl)C1=O] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-2- methylisoindolin-1-one | 517.99 |
| 351 | 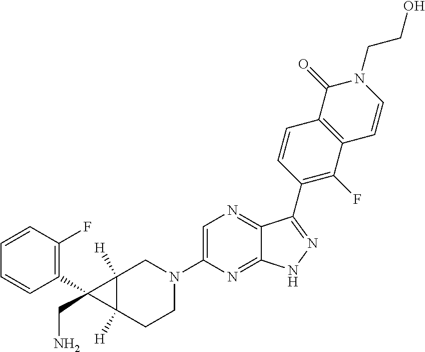 [SMILES: NC[C@]1(c2ccccc2F)[C@@H]2CCN(c3cnc4c(-c5ccc6c(=O)n(CCO)ccc6c5F)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-5-fluoro-2-(2- hydroxyethyl)isoquinolin-1(2H)-one | 543.57 |
| 352 | 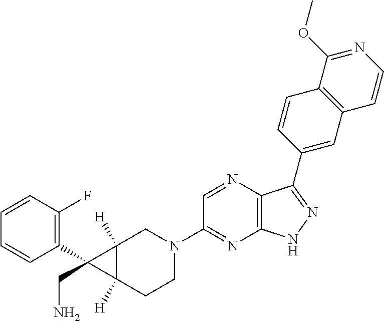 [SMILES: COc1nccc2cc(-c3n[nH]c4nc(N5CC[C@@H]6[C@H](C5)[C@@]6(CN)c5ccccc5F)cnc34)ccc12] | ((1S,6R,7R)-7-(2-fluorophenyl)-3-(3-(1- methoxyisoquinolin-6-yl)-2,4-dihydro-1H- pyrazolo[3,4-b]pyrazin-6-yl)-3- azabicyclo[4.1.0]heptan-7-yl)methanamine | 495.55 |
| 353 | 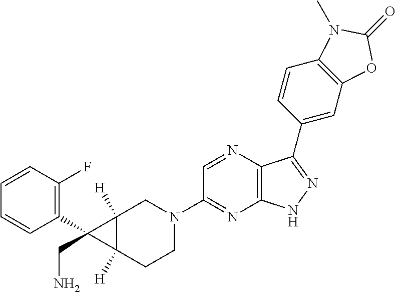 [SMILES: Cn1c(=O)oc2cc(-c3n[nH]c4nc(N5CC[C@@H]6[C@H](C5)[C@@]6(CN)c5ccccc5F)cnc34)ccc21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3- methylbenzo[d]oxazol-2(3H)-one | 485.51 |
| 354 | 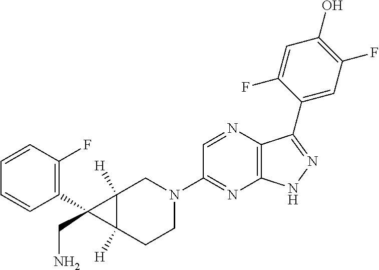 [SMILES: NC[C@]1(c2ccccc2F)[C@@H]2CCN(c3cnc4c(-c5cc(F)c(O)cc5F)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,5-difluorophenol | 466.46 |
| 355 | 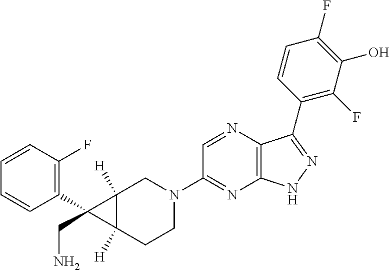 [SMILES: NC[C@]1(c2ccccc2F)[C@@H]2CCN(c3cnc4c(-c5ccc(F)c(O)c5F)n[nH]c4n3)C[C@@H]21] | 3-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,6-difluorophenol | 466.46 |
| 356 | 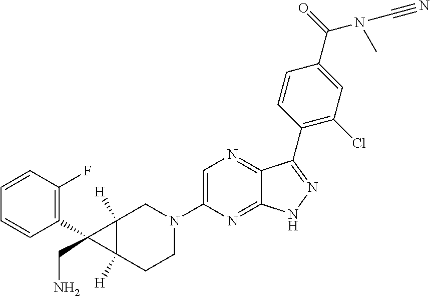 [SMILES: CN(C#N)C(=O)c1ccc(-c2n[nH]c3nc(N4CC[C@@H]5[C@H](C4)[C@@]5(CN)c4ccccc4F)cnc23)c(Cl)c1] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloro-N-cyano-N- methylbenzamide | 530.98 |
| 357 | 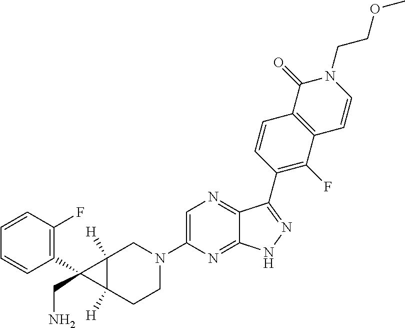 [SMILES: COCCn1ccc2c(F)c(-c3n[nH]c4nc(N5CC[C@@H]6[C@H](C5)[C@@]6(CN)c5ccccc5F)cnc34)ccc2c1=O] | 6-(6-((1S,6R,7R)-7-(aminomethyl)-7-(2- fluorophenyl)-3-azabicyclo[4.1.0]heptan-3-yl)- 114,712-pyrazolo[5,4-b]pyrazin-3-yl)-5-fluoro-2-(2- methoxyethyl)isoquinolin-1(2H)-one | 557.59 |
| 358 | 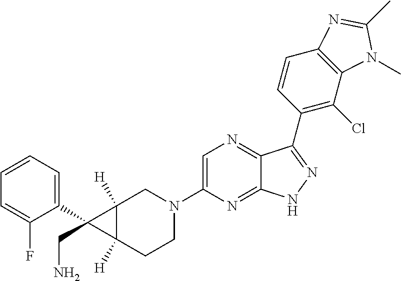 [SMILES: Cc1nc2ccc(-c3n[nH]c4nc(N5CC[C@@H]6[C@H](C5)[C@@]6(CN)c5ccccc5F)cnc34)c(Cl)c2n1C] | ((1S,6R,7R)-3-(3-(4-chloro-2,3-dimethyl-2,3- dihydro-1H-benzo[d]imidazol-5-yl)-1H-pyrazolo[3,4- b]pyrazin-6-yl)-7-(2-fluorophenyl)-3- azabicyclo[4.1.0]heptan-7-yl)methanamine | 517 |
| 359 | 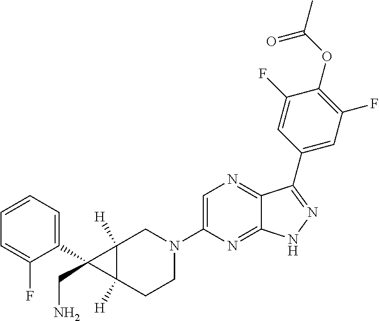 [SMILES: CC(=O)Oc1c(F)cc(-c2n[nH]c3nc(N4CC[C@@H]5[C@H](C4)[C@@]5(CN)c4ccccc4F)cnc23)cc1F] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,6-difluorophenyl acetate | 508.5 |
| 360 | 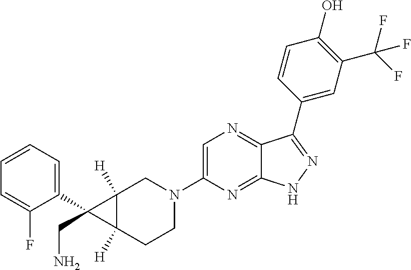 [SMILES: NC[C@]1(c2ccccc2F)[C@@H]2CCN(c3cnc4c(-c5ccc(O)c(C(F)(F)F)c5)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2- (trifluoromethyl)phenol | 498.48 |
| 361 | 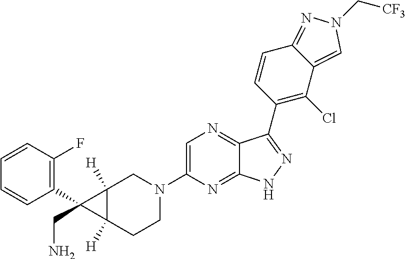 [SMILES: NC[C@]1(c2ccccc2F)[C@@H]2CCN(c3cnc4c(-c5ccc6nn(CC(F)(F)F)cc6c5Cl)n[nH]c4n3)C[C@@H]21] | ((1S,6R,7R)-3-(3-(4-chloro-2-(2,2,2-trifluoroethyl)- 2H-indazol-5-yl)-1H-pyrazolo[3,4-b]pyrazin-6-yl)-7- (2-fluorophenyl)-3-azabicyclo[4.1.0]heptan-7- yl)methanamine | 570.97 |
| 362 | 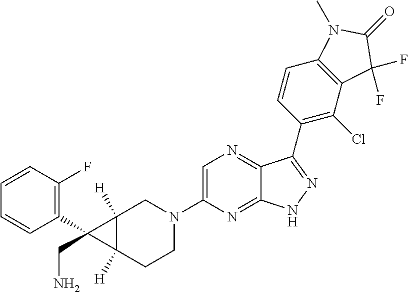 [SMILES: CN1C(=O)C(F)(F)c2c1ccc(-c1n[nH]c3nc(N4CC[C@@H]5[C@H](C4)[C@@]5(CN)c4ccccc4F)cnc13)c2Cl] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-3,3-difluoro-1- methylindolin-2-one | 553.97 |
| 363 | 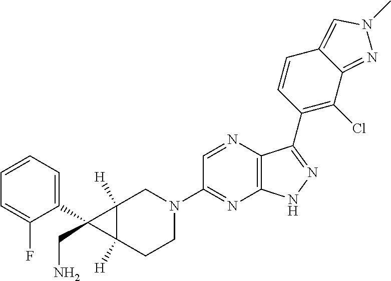 [SMILES: Cn1cc2ccc(-c3n[nH]c4nc(N5CC[C@@H]6[C@H](C5)[C@@]6(CN)c5ccccc5F)cnc34)c(Cl)c2n1] | ((1S,6R,7R)-3-(3-(7-chloro-2-methyl-2H-indazol-6- yl)-1H-pyrazolo[3,4-b]pyrazin-6-yl)-7-(2- fluorophenyl)-3-azabicyclo[4.1.0]heptan-7- yl)methanamine | 502.97 |
| 364 | 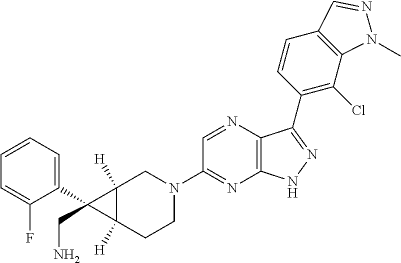 [SMILES: Cn1ncc2ccc(-c3n[nH]c4nc(N5CC[C@@H]6[C@H](C5)[C@@]6(CN)c5ccccc5F)cnc34)c(Cl)c21] | ((1S,6R,7R)-3-(3-(7-chloro-1-methyl-1H-indazol-6- yl)-1H-pyrazolo[3,4-b]pyrazin-6-yl)-7-(2- fluorophenyl)-3-azabicyclo[4.1.0]heptan-7- yl)methanamine | 502.97 |
| 365 | 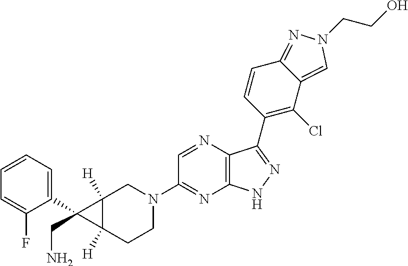 [SMILES: NC[C@]1(c2ccccc2F)[C@@H]2CCN(c3cnc4c(-c5ccc6nn(CCO)cc6c5Cl)n[nH]c4n3)C[C@@H]21] | 2-(5-(6-((1S,6R,7R)-7-(aminomethyl)-7-(2- fluorophenyl)-3-azabicyclo[4.1.0]heptan-3-yl)-2,4- dihydro-1H-pyrazolo[3,4-b]pyrazin-3-yl)-4-chloro- 2H-indazol-2-yl)ethan-1-ol | 533 |
| 366 | 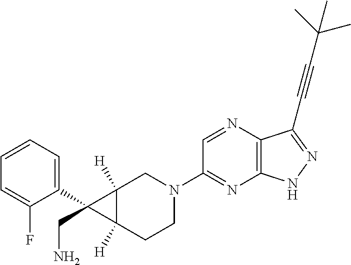 [SMILES: CC(C)(C)C#Cc1n[nH]c2nc(N3CC[C@@H]4[C@H](C3)[C@@]4(CN)c3ccccc3F)cnc12] | ((1S,6R,7R)-3-(3-(3,3-dimethylbut-1-yn-1-yl)-1H- pyrazolo[3,4-b]pyrazin-6-yl)-7-(2-fluorophenyl)-3- azabicyclo[4.1.0]heptan-7-yl)methanamine | 418.51 |
| 367 | 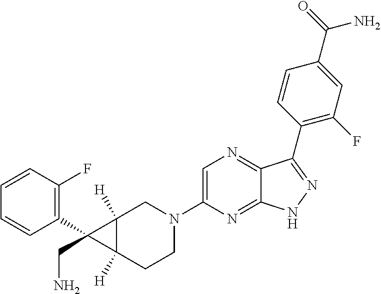 [SMILES: NC[C@]1(c2ccccc2F)[C@@H]2CCN(c3cnc4c(-c5ccc(C(N)=O)cc5F)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-fluorobenzamide | 475.49 |
| 368 | 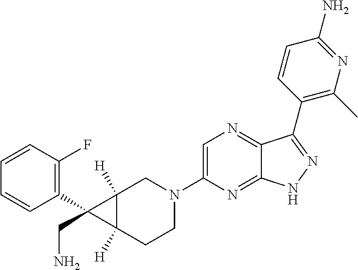 [SMILES: Cc1nc(N)ccc1-c1n[nH]c2nc(N3CC[C@@H]4[C@H](C3)[C@@]4(CN)c3ccccc3F)cnc12] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-6-methylpyridin-2- amine | 444.51 |
| 369 | 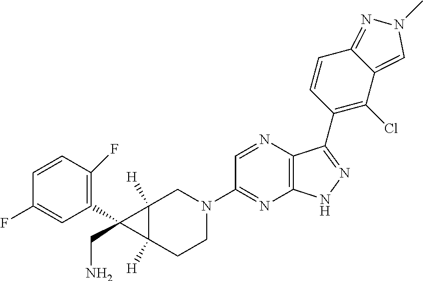 [SMILES: Cn1cc2c(Cl)c(-c3n[nH]c4nc(N5CC[C@@H]6[C@H](C5)[C@@]6(CN)c5cc(F)ccc5F)cnc34)ccc2n1] | ((1S,6R,7R)-3-(3-(4-chloro-2-methyl-2H-indazol-5- yl)-1H-pyrazolo[3,4-b]pyrazin-6-yl)-7-(2,5- difluorophenyl)-3-azabicyclo[4.1.0]heptan-7- yl)methanamine | 520.96 |
| 370 | 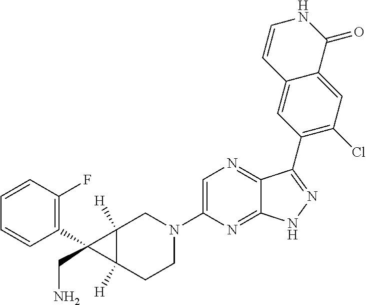 [SMILES: NC[C@]1(c2ccccc2F)[C@@H]2CCN(c3cnc4c(-c5cc6cc[nH]c(=O)c6cc5Cl)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-7-chloroisoquinolin- 1(2H)-one | 515.97 |
| 371 | 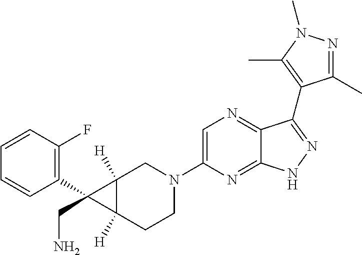 [SMILES: Cc1nn(C)c(C)c1-c1n[nH]c2nc(N3CC[C@@H]4[C@H](C3)[C@@]4(CN)c3ccccc3F)cnc12] | ((1S,6R,7R)-7-(2-fluorophenyl)-3-(3-(1,3,5- trimethyl-1H-pyrazol-4-yl)-1H-pyrazolo[3,4- b]pyrazin-6-yl)-3-azabicyclo[4.1.0]heptan-7- yl)methanamine | 446.52 |
| 372 | 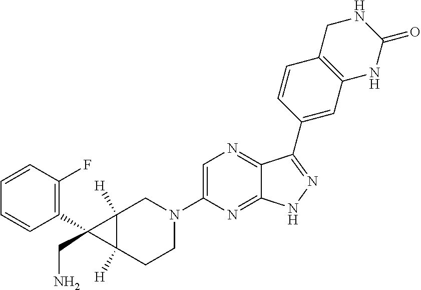 [SMILES: NC[C@]1(c2ccccc2F)[C@@H]2CCN(c3cnc4c(-c5ccc6c(c5)NC(=O)NC6)n[nH]c4n3)C[C@@H]21] | 7-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3,4-dihydroquinazolin- 2(1H)-one | 484.53 |
| 373 | 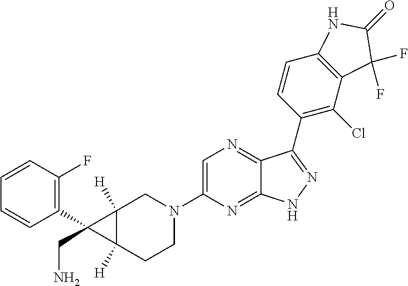 [SMILES: NC[C@]1(c2ccccc2F)[C@@H]2CCN(c3cnc4c(-c5ccc6c(c5Cl)C(F)(F)C(=O)N6)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-3,3- difluoroindolin-2-one | 539.94 |
| 374 | 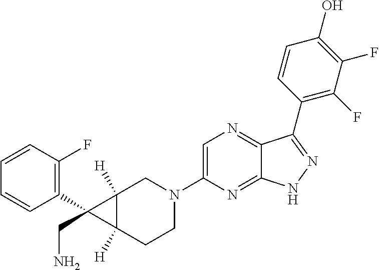 [SMILES: NC[C@]1(c2ccccc2F)[C@@H]2CCN(c3cnc4c(-c5ccc(O)c(F)c5F)n[nH]c4n3)C[C@@H]21] | 4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,3-difluorophenol | 466.46 |
| 375 | 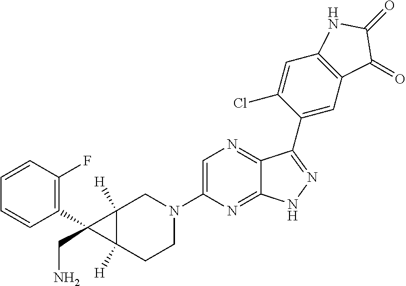 [SMILES: NC[C@]1(c2ccccc2F)[C@@H]2CCN(c3cnc4c(-c5cc6c(cc5Cl)NC(=O)C6=O)n[nH]c4n3)C[C@@H]21] | 5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-6-chloroindoline-2,3- dione | 517.94 |
| 376 | 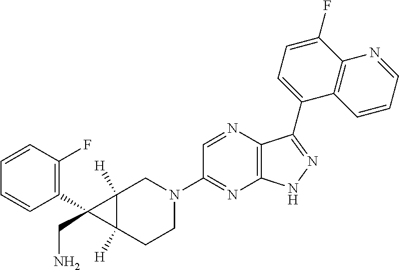 [SMILES: NC[C@]1(c2ccccc2F)[C@@H]2CCN(c3cnc4c(-c5ccc(F)c6ncccc56)n[nH]c4n3)C[C@@H]21] | ((1S,6R,7R)-7-(2-fluorophenyl)-3-(3-(8- fluoroquinolin-5-yl)-1H-pyrazolo[3,4-b]pyrazin-6- yl)-3-azabicyclo[4.1.0]heptan-7-yl)methanamine | 483.52 |
| 377 | 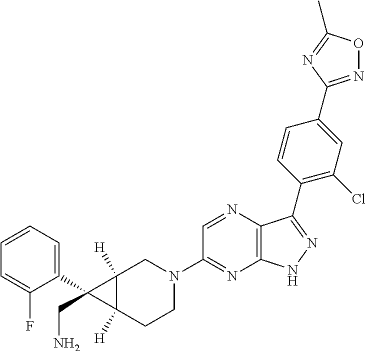 [SMILES: Cc1nc(-c2ccc(-c3n[nH]c4nc(N5CC[C@@H]6[C@H](C5)[C@@]6(CN)c5ccccc5F)cnc34)c(Cl)c2)no1] | ((1S,6R,7R)-3-(3-(2-chloro-4-(5-methyl-1,2,4- oxadiazol-3-yl)phenyl)-1H-pyrazolo[3,4-b]pyrazin-6- yl)-7-(2-fluorophenyl)-3-azabicyclo[4.1.0]heptan-7- yl)methanamine | 530.98 |
| 378 | 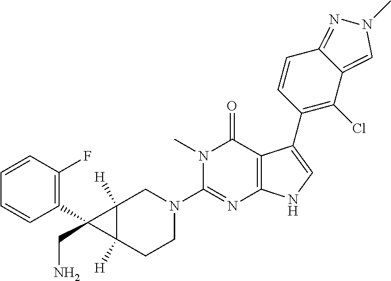 [SMILES: Cn1cc2c(Cl)c(-c3c[nH]c4nc(N5CC[C@@H]6[C@H](C5)[C@@]6(CN)c5ccccc5F)n(C)c(=O)c34)ccc2n1] | 2-((1S,6R,7R)-7-(aminomethyl)-7-(2-fluorophenyl)- 3-azabicyclo[4.1.0]heptan-3-yl)-5-(4-chloro-2- methyl-1H-214-indazol-5-yl)-3-methyl-3,7-dihydro- 4H-pyrrolo[2,3-d]pyrimidin-4-one | 532.01 |
| 379 | 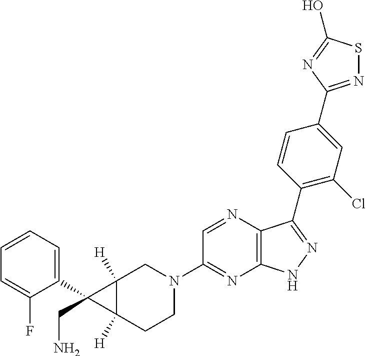 [SMILES: NC[C@]1(c2ccccc2F)[C@@H]2CCN(c3cnc4c(-c5ccc(-c6nsc(O)n6)cc5Cl)n[nH]c4n3)C[C@@H]21] | 3-(4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chlorophenyl)-1,2,4- thiadiazol-5-ol | 549.02 |
| 380 | 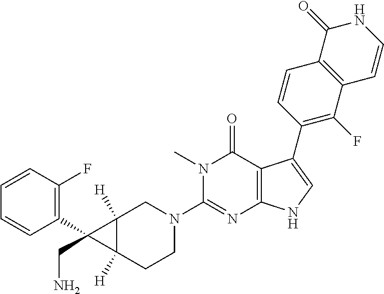 [SMILES: Cn1c(N2CC[C@@H]3[C@H](C2)[C@@]3(CN)c2ccccc2F)nc2[nH]cc(-c3ccc4c(=O)[nH]ccc4c3F)c2c1=O] | 6-(2-((1S,6R,7R)-7-(aminomethyl)-7-(2- fluorophenyl)-3-azabicyclo[4.1.0]heptan-3-yl)-3- methyl-4-oxo-4,7-dihydro-3H-pyrrolo[2,3- d]pyrimidin-5-yl)-5-fluoroisoquinolin-1(2H)-one | 528.55 |
| 381 | 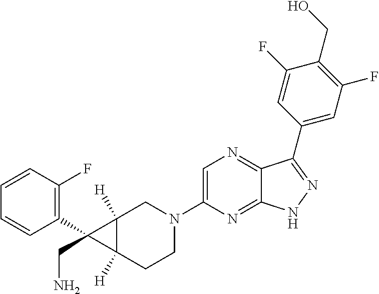 [SMILES: NC[C@]1(c2ccccc2F)[C@@H]2CCN(c3cnc4c(-c5cc(F)c(CO)c(F)c5)n[nH]c4n3)C[C@@H]21] | (4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-2,6- difluorophenyl)methanol | 480.48 |
| 382 | 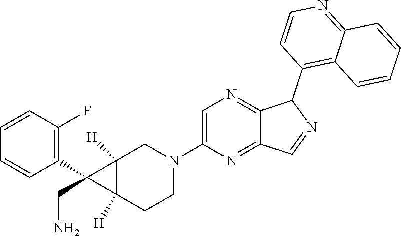 [SMILES: NC[C@]1(c2ccccc2F)[C@@H]2CCN(c3cnc4c(n3)C=NC4c3ccnc4ccccc34)C[C@@H]21] | ((1S,6R,7R)-7-(2-fluorophenyl)-3-(5-(quinolin-4-yl)- 5H-pyrrolo[2,3-b]pyrazin-2-yl)-3- azabicyclo[4.1.0]heptan-7-yl)methanamine | 464.54 |
| 383 | 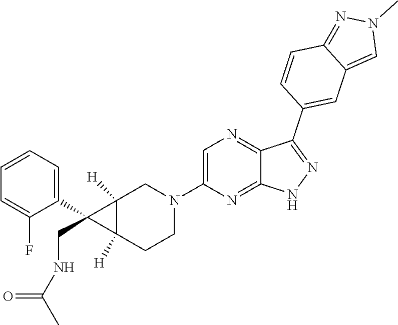 [SMILES: CC(=O)NC[C@]1(c2ccccc2F)[C@@H]2CCN(c3cnc4c(-c5ccc6nn(C)cc6c5)n[nH]c4n3)C[C@@H]21] | N-(((1S,6R,7R)-7-(2-fluorophenyl)-3-(3-(2-methyl- 2H-indazol-5-yl)-1H-pyrazolo[3,4-b]pyrazin-6-yl)-3- azabicyclo[4.1.0]heptan-7-yl)methyl)acetamide | 510.57 |
| 384 | 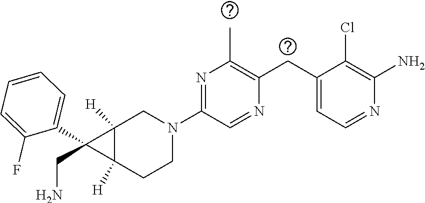 [SMILES: Cc1nc(N2CC[C@@H]3[C@H](C2)[C@@]3(CN)c2ccccc2F)cnc1Cc1ccnc(N)c1Cl] | 3-((2-amino-3-chloropyridin-4-yl)thio)-6- ((1S,6R,7R)-7-(aminomethyl)-7-(2-fluorophenyl)-3- azabicyclo[4.1.0]heptan-3-yl)pyrazin-2-amine | 471.98 |
| 385 | 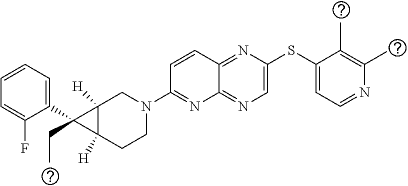 [SMILES: CC[C@]1(c2ccccc2F)[C@@H]2CCN(c3ccc4nc(Sc5ccnc(C)c5C)cnc4n3)C[C@@H]21] | 4-((6-((1S,6R,7R)-7-(aminomethyl)-7-(2- fluorophenyl)-3-azabicyclo[4.1.0]heptan-3- yl)pyrido[2,3-b]pyrazin-2-yl)thio)-3-chloropyridin-2- amine | 508.01 |
| 386 | 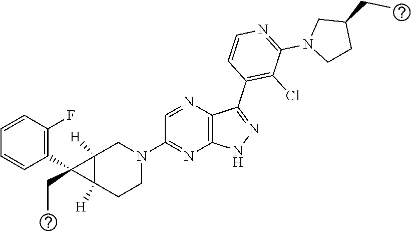 [SMILES: CC[C@H]1CCN(c2nccc(-c3n[nH]c4nc(N5CC[C@@H]6[C@H](C5)[C@@]6(CC)c5ccccc5F)cnc34)c2Cl)C1] | ((S)-1-(4-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-3-chloropyridin-2- yl)pyrrolidin-3-yl)methanol | 549.04 |
| 387 | 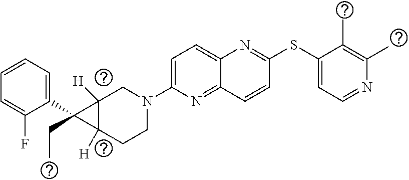 [SMILES: CC[C@]1(c2ccccc2F)C2CCN(c3ccc4nc(Sc5ccnc(C)c5C)ccc4n3)CC21] | 4-((6-((1R,6S,7S)-7-(aminomethyl)-7-(2- fluorophenyl)-3-azabicyclo[4.1.0]heptan-3-yl)-1,5- naphthyridin-2-yl)thio)-3-chloropyridin-2-amine | 507.03 |
| 388 | 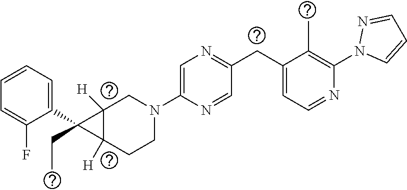 [SMILES: CC[C@]1(c2ccccc2F)C2CCN(c3cnc(Cc4ccnc(-n5cccn5)c4C)cn3)CC21] | ((1S,6R,7R)-3-(5-((3-chloro-2-(1H-pyrazol-1-yl)-1,4- dihydropyridin-4-yl)thio)pyrazin-2-yl)-7-(2- fluorophenyl)-3-azabicyclo[4.1.0]heptan-7- yl)methanamine | 508.01 |
| 389 | 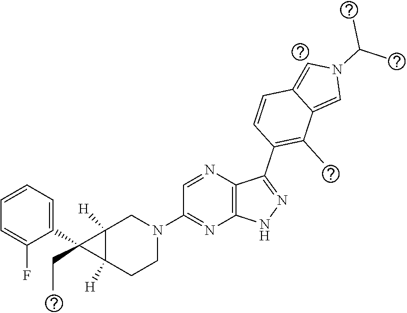 [SMILES: CC[C@]1(c2ccccc2F)[C@@H]2CCN(c3cnc4c(-c5ccc6cn(C(C)C)cc6c5C)n[nH]c4n3)C[C@@H]21] | ((1S,6R,7R)-3-(3-(2-(difluoromethyl)-4-fluoro-2H- indazol-5-yl)-1H-pyrazolo[3,4-b]pyrazin-6-yl)-7-(2- fluorophenyl)-3-azabicyclo[4.1.0]heptan-7- yl)methanamine | 522.5 |
| 390 | 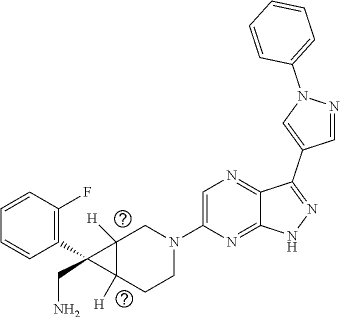 [SMILES: NC[C@]1(c2ccccc2F)C2CCN(c3cnc4c(-c5cnn(-c6ccccc6)c5)n[nH]c4n3)CC21] | ((1S,6R,7R)-7-(2-fluorophenyl)-3-(3-(1-phenyl-1H- pyrazol-4-yl)-1H-pyrazolo[3,4-b]pyrazin-6-yl)-3- azabicyclo[4.1.0]heptan-7-yl)methanamine | 480.54 |
| 391 | 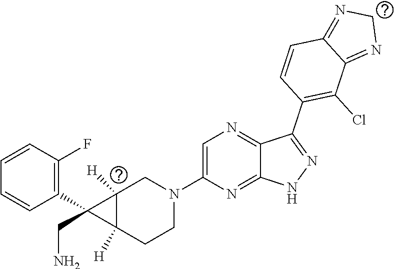 [SMILES: NC[C@]1(c2ccccc2F)[C@@H]2CCN(c3cnc4c(-c5ccc6c(c5Cl)=NCN=6)n[nH]c4n3)C[C@@H]21] | ((1S,6R,7R)-3-(3-(4-chloro-1,4- dihydrobenzo[c][1,2,5]thiadiazol-5-yl)-1H- pyrazolo[3,4-b]pyrazin-6-yl)-7-(2-fluorophenyl)-3- azabicyclo[4.1.0]heptan-7-yl)methanamine | 506.99 |
| 392 | 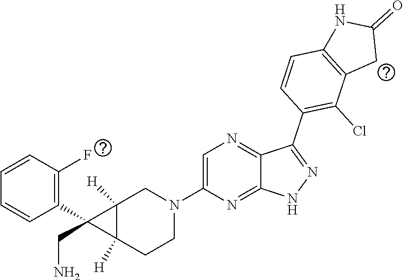 [SMILES: NC[C@]1(c2ccccc2F)[C@@H]2CCN(c3cnc4c(-c5ccc6c(c5Cl)CC(=O)N6)n[nH]c4n3)C[C@@H]21] | 6-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-7- chlorobenzo[d]thiazol-2(3H)-one | 522 |
| 393 | 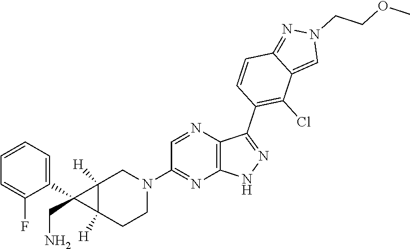 [SMILES: COCCn1cc2c(Cl)c(-c3n[nH]c4nc(N5CC[C@@H]6[C@H](C5)[C@@]6(CN)c5ccccc5F)cnc34)ccc2n1] | ((1S,6R,7R)-3-(3-(4-chloro-2-(2-methoxyethyl)-2H- indazol-5-yl)-1H-pyrazolo[3,4-b]pyrazin-6-yl)-7-(2- fluorophenyl)-3-azabicyclo[4.1.0]heptan-7- yl)methanamine | 547.03 |
| 394 | 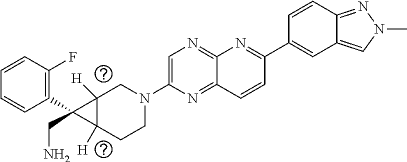 [SMILES: Cn1cc2cc(-c3ccc4nc(N5CCC6C(C5)[C@@]6(CN)c5ccccc5F)cnc4n3)ccc2n1] | ((1S,6R,7R)-7-(2-fluorophenyl)-3-(6-(2-methyl-2H- indazol-5-yl)-1,5-naphthyridin-2-yl)-3- azabicyclo[4.1.0]heptan-7-yl)methanamine | 478.56 |
| 395 | 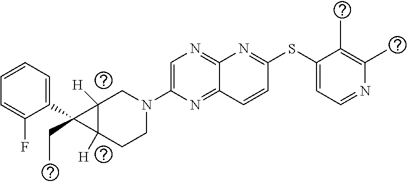 [SMILES: CC[C@]1(c2ccccc2F)C2CCN(c3cnc4nc(Sc5ccnc(C)c5C)ccc4n3)CC21] | 4-((2-((1S,6R,7R)-7-(aminomethyl)-7-(2- fluorophenyl)-3-azabicyclo[4.1.0]heptan-3- yl)pyrido[2,3-b]pyrazin-6-yl)thio)-3-chloropyridin-2- amine | 508.01 |
| 396 | 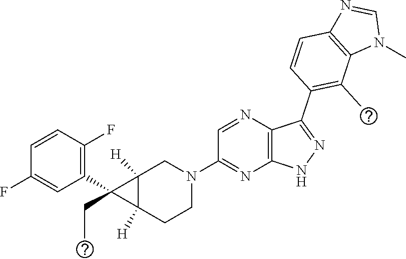 [SMILES: CC[C@]1(c2cc(F)ccc2F)[C@@H]2CCN(c3cnc4c(-c5ccc6ncn(C)c6c5C)n[nH]c4n3)C[C@@H]21] | ((1S,6R,7R)-3-(3-(7-chloro-1-methyl-1H- benzo[d]imidazol-6-yl)-1H-pyrazolo[3,4-b]pyrazin-6- yl)-7-(2,5-difluorophenyl)-3-azabicyclo[4.1.0]heptan- 7-yl)methanamine | 520.96 |
| 397 | 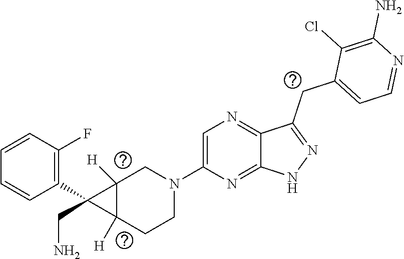 [SMILES: NC[C@]1(c2ccccc2F)C2CCN(c3cnc4c(Cc5ccnc(N)c5Cl)n[nH]c4n3)CC21] | 4-((6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)thio)-3-chloropyridin-2- amine | 496.99 |
| 398 | 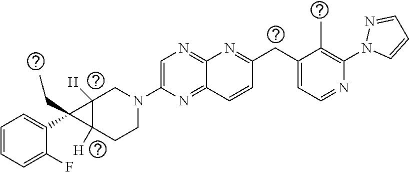 [SMILES: CC[C@]1(c2ccccc2F)C2CCN(c3cnc4nc(Cc5ccnc(-n6cccn6)c5C)ccc4n3)CC21] | ((1S,6R,7R)-3-(6-((3-chloro-2-(1H-pyrazol-1- yl)pyridin-4-yl)thio)pyrido[2,3-b]pyrazin-2-yl)-7-(2- fluorophenyl)-3-azabicyclo[4.1.0]heptan-7- yl)methanamine | 559.06 |
| 399 | 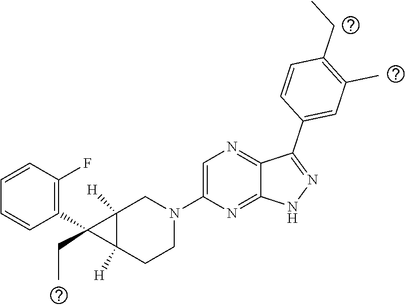 [SMILES: CCc1ccc(-c2n[nH]c3nc(N4CC[C@@H]5[C@H](C4)[C@@]5(CC)c4ccccc4F)cnc23)cc1C] | ((1S,6R,7R)-3-(3-(3-fluoro-4-methoxyphenyl)-1H- pyrazolo[3,4-b]pyrazin-6-yl)-7-(2-fluorophenyl)-3- azabicyclo[4.1.0]heptan-7-yl)methanamine | 462.49 |
| 400 | 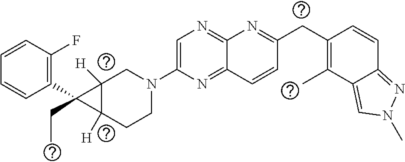 [SMILES: CC[C@]1(c2ccccc2F)C2CCN(c3cnc4nc(Cc5ccc6nn(C)cc6c5C)ccc4n3)CC21] | ((1S,6R,7R)-3-(6-((4-chloro-2-methyl-2H-indazol-5- yl)thio)pyrido[2,3-b]pyrazin-2-yl)-7-(2- fluorophenyl)-3-azabicyclo[4.1.0]heptan-7- yl)methanamine | 546.06 |
| 401 | 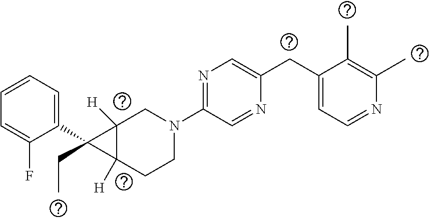 [SMILES: CC[C@]1(c2ccccc2F)C2CCN(c3cnc(Cc4ccnc(C)c4C)cn3)CC21] | 4-((5-((1S,6R,7R)-7-(aminomethyl)-7-(2- fluorophenyl)-3-azabicyclo[4.1.0]heptan-3- yl)pyrazin-2-yl)thio)-3-chloropyridin-2-amine | 456.97 |
| 402 | 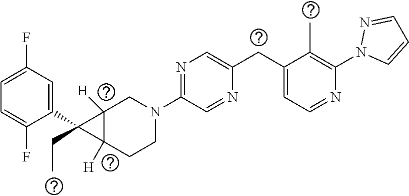 [SMILES: CC[C@]1(c2cc(F)ccc2F)C2CCN(c3cnc(Cc4ccnc(-n5cccn5)c4C)cn3)CC21] | ((1S,6R,7R)-3-(5-((3-chloro-2-(1H-pyrazol-1- yl)pyridin-4-yl)thio)pyrazin-2-yl)-7-(2,5- difluorophenyl)-3-azabicyclo[4.1.0]heptan-7- yl)methanamine | 526 |
| 403 | 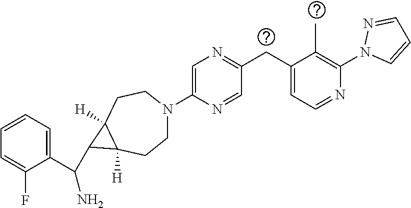 [SMILES: Cc1c(Cc2cnc(N3CC[C@@H]4C(C(N)c5ccccc5F)[C@@H]4CC3)cn2)ccnc1-n1cccn1] | ((1R,7S)-4-(5-((3-chloro-2-(1H-pyrazol-1-yl)pyridin- 4-yl)thio)pyrazin-2-yl)-8-(2-fluorophenyl)-4- azabicyclo[5.1.0]octan-8-yl)methanamine | 522.04 |
| 404 | 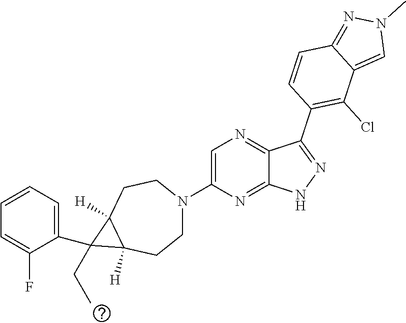 [SMILES: CCC1(c2ccccc2F)[C@@H]2CCN(c3cnc4c(-c5ccc6nn(C)cc6c5Cl)n[nH]c4n3)CC[C@@H]21] | ((1R,7S)-4-(3-(4-chloro-2-methyl-2H-indazol-5-yl)- 1H-pyrazolo[3,4-b]pyrazin-6-yl)-8-(2-fluorophenyl)- 4-azabicyclo[5.1.0]octan-8-yl)methanamine | 517 |
| 405 | 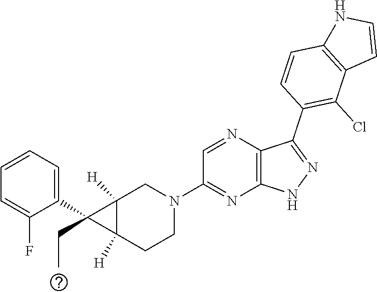 [SMILES: CC[C@]1(c2ccccc2F)[C@@H]2CCN(c3cnc4c(-c5ccc6[nH]ccc6c5Cl)n[nH]c4n3)C[C@@H]21] | ((1S,6R,7R)-3-(3-(4-chloro-1H-indol-5-yl)-1H- pyrazolo[3,4-b]pyrazin-6-yl)-7-(2-fluorophenyl)-3- azabicyclo[4.1.0]heptan-7-yl)methanamine | 487.96 |
| 406 | 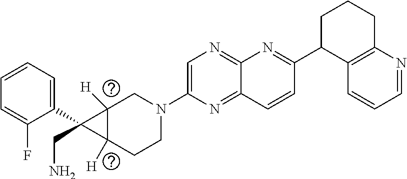 [SMILES: NC[C@]1(c2ccccc2F)C2CCN(c3cnc4nc(C5CCCc6ncccc65)ccc4n3)CC21] | ((1S,6R,7R)-3-(6-(3,4-dihydro-1,5-naphthyridin- 1(2H)-yl)pyrido[2,3-b]pyrazin-2-yl)-7-(2- fluorophenyl)-3-azabicyclo[4.1.0]heptan-7- yl)methanamine | 481.57 |
| 407 | 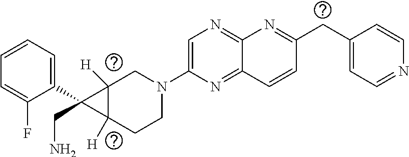 [SMILES: NC[C@]1(c2ccccc2F)C2CCN(c3cnc4nc(Cc5ccncc5)ccc4n3)CC21] | ((1S,6R,7R)-7-(2-fluorophenyl)-3-(6-(pyridin-4- ylthio)-3,4-dihydropyrido[2,3-b]pyrazin-2-yl)-3- azabicyclo[4.1.0]heptan-7-yl)methanamine | 458.55 |
| 408 | 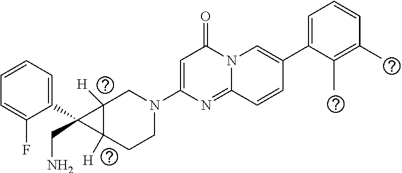 [SMILES: Cc1cccc(-c2ccc3nc(N4CCC5C(C4)[C@@]5(CN)c4ccccc4F)cc(=O)n3c2)c1C] | 2-((1S,6R,7R)-7-(aminomethyl)-7-(2-fluorophenyl)- 3-azabicyclo[4.1.0]heptan-3-yl)-7-(2,3- dichlorophenyl)-4H-pyrido[1,2-a]pyrimidin-4-one | 509.4 |
| 409 | 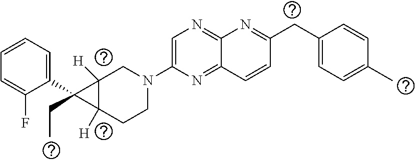 [SMILES: CC[C@]1(c2ccccc2F)C2CCN(c3cnc4nc(Cc5ccc(C)cc5)ccc4n3)CC21] | ((1S,6R,7R)-3-(6-(4-fluorophenoxy)pyrido[2,3- b]pyrazin-2-yl)-7-(2-fluorophenyl)-3- azabicyclo[4.1.0]heptan-7-yl)methanamine | 459.49 |
| 410 | 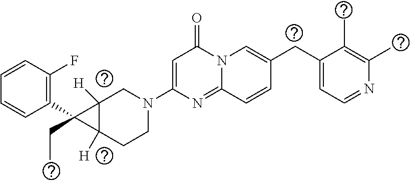 [SMILES: CC[C@]1(c2ccccc2F)C2CCN(c3cc(=O)n4cc(Cc5ccnc(C)c5C)ccc4n3)CC21] | 7-((2-amino-3-chloropyridin-4-yl)thio)-2- ((1S,6R,7R)-7-(aminomethyl)-7-(2-fluorophenyl)-3- azabicyclo[4.1.0]heptan-3-yl)-4H-pyrido[1,2- a]pyrimidin-4-one | 523.02 |
| 411 | 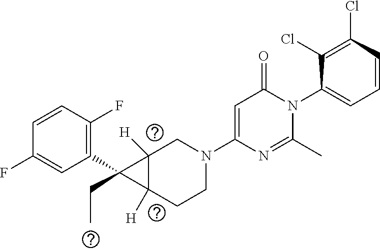 | 6-((1S,6R,7R)-7-(aminomethyl)-7-(2,5- difluorophenyl)-3-azabicyclo[4.1.0]heptan-3-yl)-3- (2,3-dichlorophenyl)-2-methylpyrimidin-4(3H)-one | 491.36 |
| 412 | 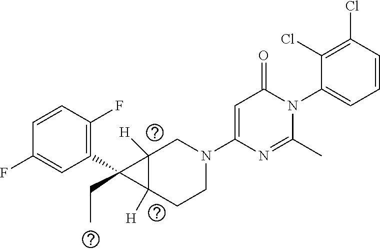 [SMILES: CC[C@]1(c2cc(F)ccc2F)C2CCN(c3cc(=O)n(-c4cccc(Cl)c4Cl)c(C)n3)CC21] | 6-((1S,6R,7R)-7-(aminomethyl)-7-(2,5- difluorophenyl)-3-azabicyclo[4.1.0]heptan-3-yl)-3- (2,3-dichlorophenyl)-2-methylpyrimidin-4(3H)-one | 491.36 |
| 413 | 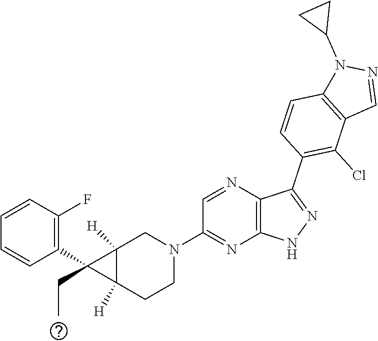 [SMILES: CC[C@]1(c2ccccc2F)[C@@H]2CCN(c3cnc4c(-c5ccc6c(cnn6C6CC6)c5Cl)n[nH]c4n3)C[C@@H]21] | ((1S,6R,7R)-3-(3-(4-chloro-1-cyclopropyl-1H- indazol-5-yl)-1H-pyrazolo[3,4-b]pyrazin-6-yl)-7-(2- fluorophenyl)-3-azabicyclo[4.1.0]heptan-7- yl)methanamine | 529.01 |
| 414 | 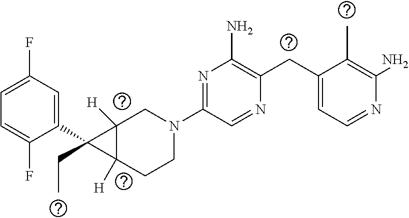 [SMILES: CC[C@]1(c2cc(F)ccc2F)C2CCN(c3cnc(Cc4ccnc(N)c4C)c(N)n3)CC21] | 3-((2-amino-3-chloropyridin-4-yl)thio)-6- ((1S,6R,7R)-7-(aminomethyl)-7-(2,5- difluorophenyl)-3-azabicyclo[4.1.0]heptan-3- yl)pyrazin-2-amine | 489.97 |
| 415 | 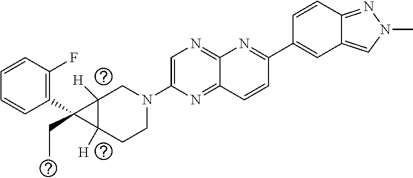 [SMILES: CC[C@]1(c2ccccc2F)C2CCN(c3cnc4nc(-c5ccc6nn(C)cc6c5)ccc4n3)CC21] | ((1S,6R,7R)-7-(2-fluorophenyl)-3-(6-(2-methyl-2H- indazol-5-yl)pyrido[2,3-b]pyrazin-2-yl)-3- azabicyclo[4.1.0]heptan-7-yl)methanamine | 479.55 |
| 416 | 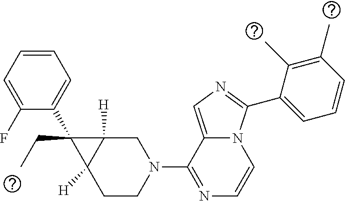 [SMILES: CC[C@]1(c2ccccc2F)[C@@H]2CCN(c3nccn4c(-c5cccc(C)c5C)ncc34)C[C@@H]21] | ((1S,6R,7R)-3-(3-(2,3-dichlorophenyl)imidazo[1,5- a]pyrazin-8-yl)-7-(2-fluorophenyl)-3- azabicyclo[4.1.0]heptan-7-yl)methanamine | 482.38 |
| 417 | 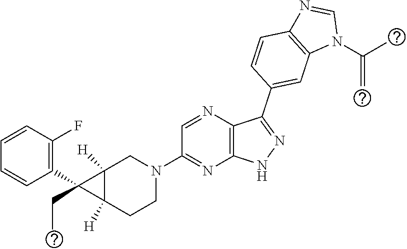 [SMILES: C=C(C)n1cnc2ccc(-c3n[nH]c4nc(N5CC[C@@H]6[C@H](C5)[C@@]6(CC)c5ccccc5F)cnc34)cc21] | ((1S,6R,7R)-3-(3-(1-(difluoromethyl)-1H- benzo[d]imidazol-6-yl)-1H-pyrazolo[3,4-b]pyrazin-6- yl)-7-(2-fluorophenyl)-3-azabicyclo[4.1.0]heptan-7- yl)methanamine | 504.51 |
| 418 | 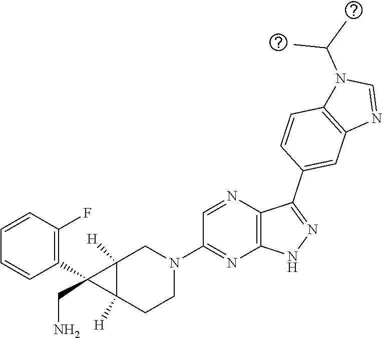 [SMILES: CC(C)n1cnc2cc(-c3n[nH]c4nc(N5CC[C@@H]6[C@H](C5)[C@@]6(CN)c5ccccc5F)cnc34)ccc21] | ((1S,6R,7R)-3-(3-(1-(difluoromethyl)-1H- benzo[d]imidazol-5-yl)-1H-pyrazolo[3,4-b]pyrazin-6- yl)-7-(2-fluorophenyl)-3-azabicyclo[4.1.0]heptan-7- yl)methanamine | 504.51 |
| 419 | 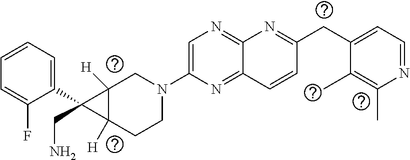 [SMILES: Cc1nccc(Cc2ccc3nc(N4CCC5C(C4)[C@@]5(CN)c4ccccc4F)cnc3n2)c1C] | ((1S,6R,7R)-3-(6-((3-fluoro-2-methylpyridin-4- yl)thio)pyrido[2,3-b]pyrazin-2-yl)-7-(2- fluorophenyl)-3-azabicyclo[4.1.0]heptan-7- yl)methanamine | 490.57 |
| 420 | 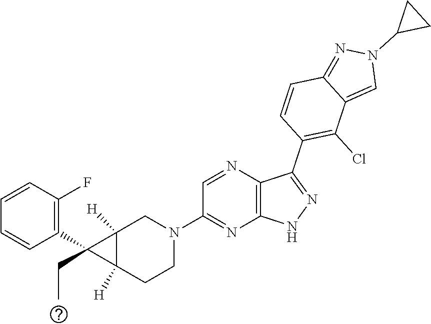 [SMILES: CC[C@]1(c2ccccc2F)[C@@H]2CCN(c3cnc4c(-c5ccc6nn(C7CC7)cc6c5Cl)n[nH]c4n3)C[C@@H]21] | ((1S,6R,7R)-3-(3-(4-chloro-2-cyclopropyl-2H- indazol-5-yl)-1H-pyrazolo[3,4-b]pyrazin-6-yl)-7-(2- fluorophenyl)-3-azabicyclo[4.1.0]heptan-7- yl)methanamine | 529.01 |
| 421 | 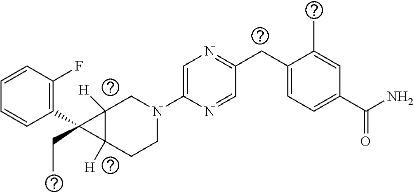 [SMILES: CC[C@]1(c2ccccc2F)C2CCN(c3cnc(Cc4ccc(C(N)=O)cc4C)cn3)CC21] | 4-((5-((1S,6R,7R)-7-(aminomethyl)-7-(2- fluorophenyl)-3-azabicyclo[4.1.0]heptan-3- yl)pyrazin-2-yl)thio)-3-chlorobenzamide | 483.99 |
| 422 | 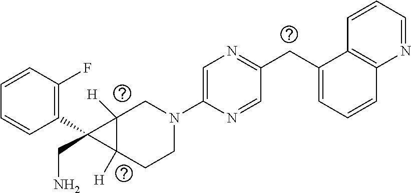 [SMILES: NC[C@]1(c2ccccc2F)C2CCN(c3cnc(Cc4cccc5ncccc45)cn3)CC21] | ((1S,6R,7R)-7-(2-fluorophenyl)-3-(5-(quinolin-5- ylthio)pyrazin-2-yl)-3-azabicyclo[4.1.0]heptan-7- yl)methanamine | 457.57 |
| 423 | 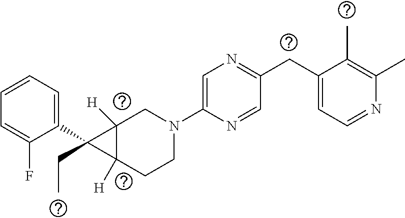 [SMILES: CC[C@]1(c2ccccc2F)C2CCN(c3cnc(Cc4ccnc(C)c4C)cn3)CC21] | ((1S,6R,7R)-3-(5-((3-fluoro-2-methylpyridin-4- yl)thio)pyrazin-2-yl)-7-(2-fluorophenyl)-3- azabicyclo[4.1.0]heptan-7-yl)methanamine | 439.52 |
| 424 | 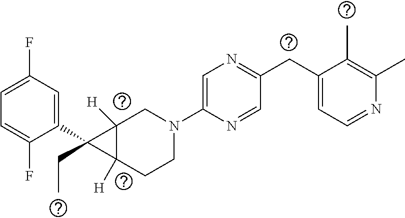 [SMILES: CC[C@]1(c2cc(F)ccc2F)C2CCN(c3cnc(Cc4ccnc(C)c4C)cn3)CC21] | ((1S,6R,7R)-7-(2,5-difluorophenyl)-3-(5-((3-fluoro- 2-methylpyridin-4-yl)thio)pyrazin-2-yl)-3- azabicyclo[4.1.0]heptan-7-yl)methanamine | 457.51 |
| 425 | 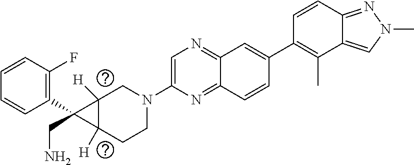 [SMILES: Cc1c(-c2ccc3nc(N4CCC5C(C4)[C@@]5(CN)c4ccccc4F)cnc3c2)ccc2nn(C)cc12] | ((1S,6R,7R)-3-(6-(2,4-dimethyl-2H-indazol-5- yl)pyrido[2,3-b]pyrazin-2-yl)-7-(2-fluorophenyl)-3- azabicyclo[4.1.0]heptan-7-yl)methanamine | 493.58 |
| 426 | 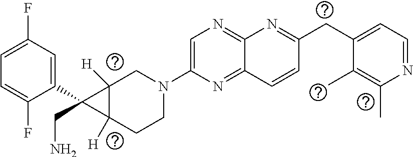 [SMILES: Cc1nccc(Cc2ccc3nc(N4CCC5C(C4)[C@@]5(CN)c4cc(F)ccc4F)cnc3n2)c1C] | ((1S,6R,7R)-7-(2,5-difluorophenyl)-3-(6-((3-fluoro- 2-methylpyridin-4-yl)thio)pyrido[2,3-b]pyrazin-2-yl)- 3-azabicyclo[4.1.0]heptan-7-yl)methanamine | 508.56 |
| 427 | 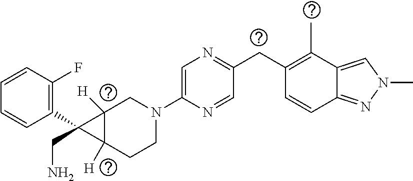 [SMILES: Cc1c(Cc2cnc(N3CCC4C(C3)[C@@]4(CN)c3ccccc3F)cn2)ccc2nn(C)cc12] | ((1S,6R,7R)-3-(5-((4-chloro-2-methyl-2H-indazol-5- yl)thio)pyrazin-2-yl)-7-(2-fluorophenyl)-3- azabicyclo[4.1.0]heptan-7-yl)methanamine | 495.01 |
| 428 | 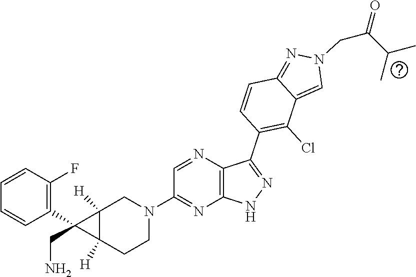 [SMILES: CC(C)C(=O)Cn1cc2c(Cl)c(-c3n[nH]c4nc(N5CC[C@@H]6[C@H](C5)[C@@]6(CN)c5ccccc5F)cnc34)ccc2n1] | 2-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-2H-indazol-2- yl)-N,N-dimethylacetamide | 574.05 |
| 429 | 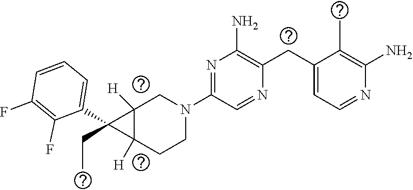 [SMILES: CC[C@]1(c2cccc(F)c2F)C2CCN(c3cnc(Cc4ccnc(N)c4C)c(N)n3)CC21] | 3-((2-amino-3-chloropyridin-4-yl)thio)-6- ((1S,6R,7R)-7-(aminomethyl)-7-(2,3- difluorophenyl)-3-azabicyclo[4.1.0]heptan-3- yl)pyrazin-2-amine | 489.97 |
| 430 | 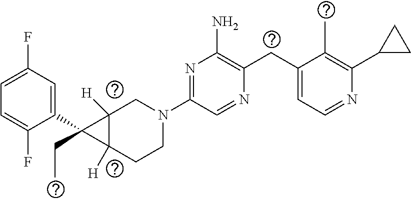 [SMILES: CC[C@]1(c2cc(F)ccc2F)C2CCN(c3cnc(Cc4ccnc(C5CC5)c4C)c(N)n3)CC21] | 6-((1S,6R,7R)-7-(aminomethyl)-7-(2,5- difluorophenyl)-3-azabicyclo[4.1.0]heptan-3-yl)-3- ((3-chloro-2-cyclopropylpyridin-4-yl)thio)pyrazin-2- amine | 515.02 |
| 431 | 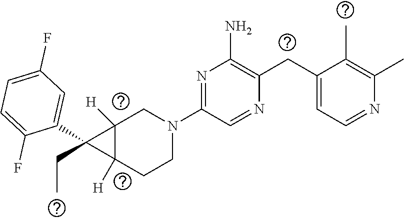 [SMILES: CC[C@]1(c2cc(F)ccc2F)C2CCN(c3cnc(Cc4ccnc(C)c4C)c(N)n3)CC21] | 6-((1S,6R,7R)-7-(aminomethyl)-7-(2,5- difluorophenyl)-3-azabicyclo[4.1.0]heptan-3-yl)-3- ((3-fluoro-2-methylpyridin-4-yl)thio)pyrazin-2-amine | 472.53 |
| 432 | 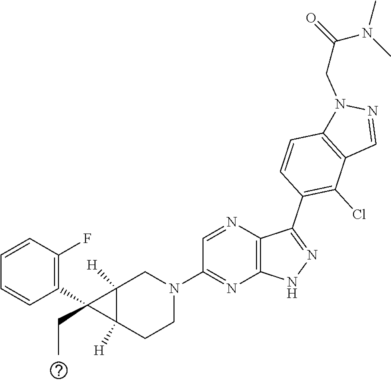 [SMILES: CC[C@]1(c2ccccc2F)[C@@H]2CCN(c3cnc4c(-c5ccc6c(cnn6CC(=O)N(C)C)c5Cl)n[nH]c4n3)C[C@@H]21] | 2-(5-(6-((1S,6R,7R)-7-(aminomethyl)-7-(2- fluorophenyl)-3-azabicyclo[4.1.0]heptan-3-yl)-1H- pyrazolo[3,4-b]pyrazin-3-yl)-4-chloro-1H-indazol-1- yl)-N,N-dimethylacetamide | 574.05 |
| 433 | 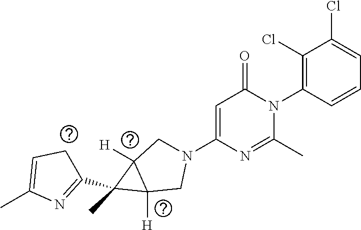 | 6-((1R,5S,6r)-6-(aminomethyl)-6-(4-methylthiazol-2- yl)-3-azabicyclo[3.1.0]hexan-3-yl)-3-(2,3- dichlorophenyl)-2-methylpyrimidin-4(3H)-one | 462.4 |
| 434 | 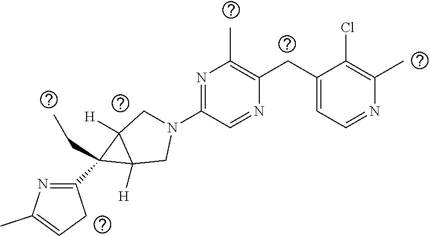 | 3-((2-amino-3-chloropyridin-4-yl)thio)-6-((1R,5S,6r)- 6-(aminomethyl)-6-(4-methylthiazol-2-yl)-3- azabicyclo[3.1.0]hexan-3-yl)pyrazin-2-amine | 461.01 |
| 435 | 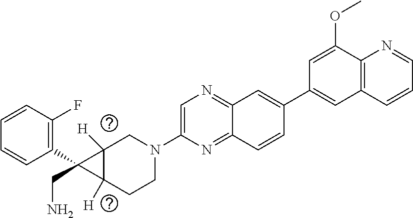 [SMILES: COc1cc(-c2ccc3nc(N4CCC5C(C4)[C@@]5(CN)c4ccccc4F)cnc3c2)cc2cccnc12] | ((1S,6R,7R)-7-(2-fluorophenyl)-3-(6-(8- methoxyquinolin-6-yl)pyrido[2,3-b]pyrazin-2-yl)-3- azabicyclo[4.1.0]heptan-7-yl)methanamine | 506.57 |
| 436 | 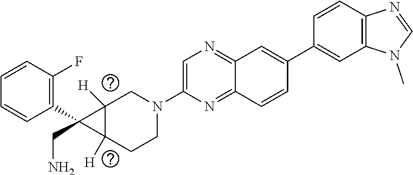 [SMILES: Cn1cnc2ccc(-c3ccc4nc(N5CCC6C(C5)[C@@]6(CN)c5ccccc5F)cnc4c3)cc21] | ((1S,6R,7R)-7-(2-fluorophenyl)-3-(6-(1-methyl-1H- benzo[d]imidazol-6-yl)pyrido[2,3-b]pyrazin-2-yl)-3- azabicyclo[4.1.0]heptan-7-yl)methanamine | 479.55 |
| 437 | 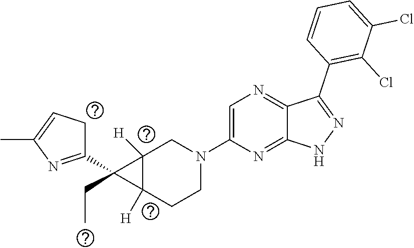 [SMILES: CC[C@]1(C2=NC(C)=CC2)C2CCN(c3cnc4c(-c5cccc(Cl)c5Cl)n[nH]c4n3)CC21] | ((1R,6S,7R)-3-(3-(2,3-dichlorophenyl)-1H- pyrazolo[3,4-b]pyrazin-6-yl)-7-(4-methylthiazol-2- yl)-3-azabicyclo[4.1.0]heptan-7-yl)methanamine | 486.42 |
| 438 | 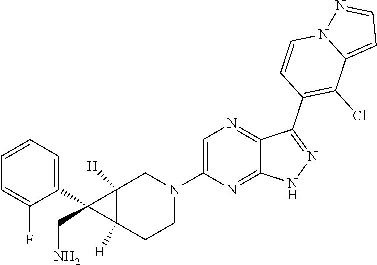 [SMILES: NC[C@]1(c2ccccc2F)[C@@H]2CCN(c3cnc4c(-c5ccn6nccc6c5Cl)n[nH]c4n3)C[C@@H]21] | ((1S,6R,7R)-3-(3-(4-chloropyrazolo[1,5-a]pyridin-5- yl)-1H-pyrazolo[3,4-b]pyrazin-6-yl)-7-(2- fluorophenyl)-3-azabicyclo[4.1.0]heptan-7- yl)methanamine | 488.95 |
| 439 | 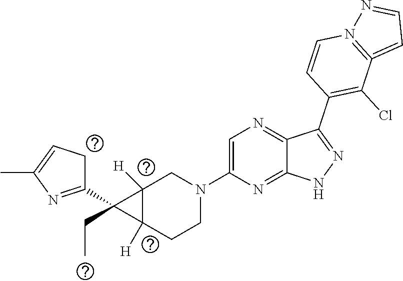 | ((1S,6R,7S)-3-(3-(4-chloropyrazolo[1,5-a]pyridin-5- yl)-1H-pyrazolo[3,4-b]pyrazin-6-yl)-7-(4- methylthiazol-2-yl)-3-azabicyclo[4.1.0]heptan-7- yl)methanamine | 492 |
| 440 | 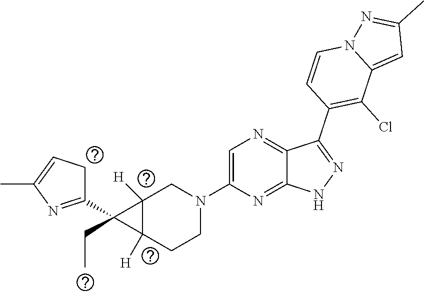 | ((1S,6R,7S)-3-(3-(4-chloro-2-methylpyrazolo[1,5- a]pyridin-5-yl)-1H-pyrazolo[3,4-b]pyrazin-6-yl)-7- (4-methylthiazol-2-yl)-3-azabicyclo[4.1.0]heptan-7- yl)methanamine | 506.03 |
| 441 | 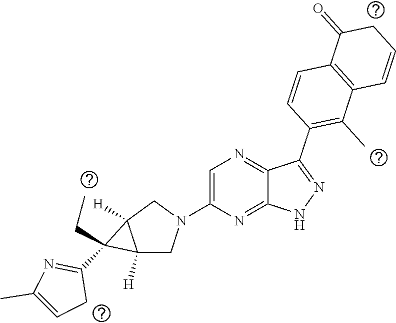 [SMILES: CC[C@]1(C2=NC(C)=CC2)[C@@H]2CN(c3cnc4c(-c5ccc6c(c5C)C=CCC6=O)n[nH]c4n3)C[C@@H]21] | 6-(6-((1R,5S,6r)-6-(aminomethyl)-6-(4- methylthiazol-2-yl)-3-azabicyclo[3.1.0]hexan-3-yl)- 4,7-dihydro-1H-pyrazolo[3,4-b]pyrazin-3-yl)-5- fluoroisoquinolin-1(2H)-one | 488.54 |
| 442 | 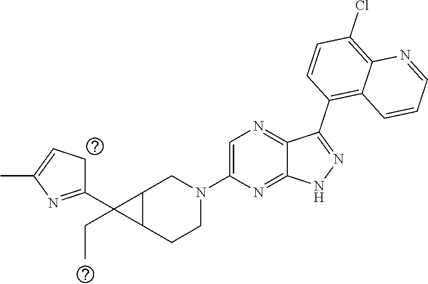 [SMILES: CCC1(C2=NC(C)=CC2)C2CCN(c3cnc4c(-c5ccc(Cl)c6ncccc56)n[nH]c4n3)CC21] | (3-(3-(8-chloroquinolin-5-yl)-1H-pyrazolo[3,4- b]pyrazin-6-yl)-7-(4-methylthiazol-2-yl)-3- azabicyclo[4.1.0]heptan-7-yl)methanamine | 503.02 |
| 443 | 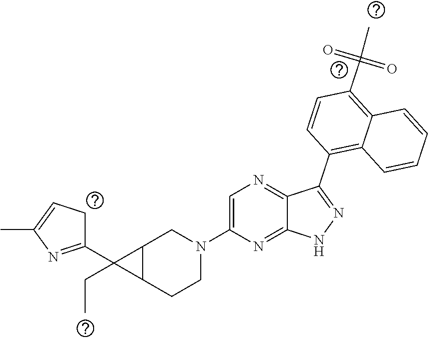 | 4-(6-(7-(aminomethyl)-7-(4-methyl-2,3- dihydrothiazol-2-yl)-3-azabicyclo[4.1.0]heptan-3-yl)- 4,7-dihydro-1H-pyrazolo[3,4-b]pyrazin-3- yl)naphthalene-1-sulfonyl fluoride | 549.64 |
| 444 | 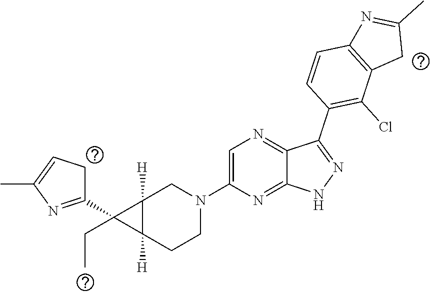 | ((1S,6R,7S)-3-(3-(7-chloro-2-methylbenzo[d]thiazol- 6-yl)-1H-pyrazolo[3,4-b]pyrazin-6-yl)-7-(4- methylthiazol-2-yl)-3-azabicyclo[4.1.0]heptan-7- yl)methanamine | 523.08 |
| 445 | 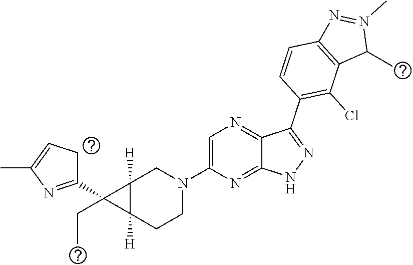 | ((1S,6R,7S)-3-(3-(3,4-dichloro-2-methyl-2H-indazol- 5-yl)-1H-pyrazolo[3,4-b]pyrazin-6-yl)-7-(4- methylthiazol-2-yl)-3-azabicyclo[4.1.0]heptan-7- yl)methanamine | 540.47 |
| 446 | 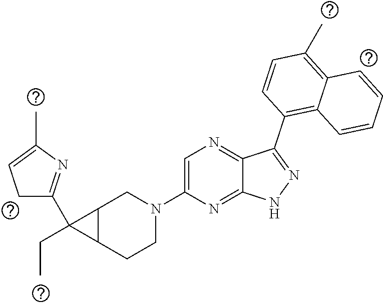 [SMILES: CCC1(C2=NC(C)=CC2)C2CCN(c3cnc4c(-c5ccc(C)c6ccccc56)n[nH]c4n3)CC21] | (7-(4-chlorothiazol-2-yl)-3-(3-(8-fluoroquinolin-5- yl)-4H-114-pyrazolo[4,5-b]pyrazin-6-yl)-3- azabicyclo[4.1.0]heptan-7-yl)methanamine | 506.99 |
| 447 | 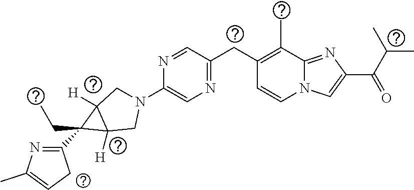 | 7-((5-((1R,5S,6r)-6-(aminomethyl)-6-(4- methylthiazol-2-yl)-3-azabicyclo[3.1.0]hexan-3- yl)pyrazin-2-yl)thio)-8-chloro-N,N- dimethylimidazo[1,2-a]pyridine-2-carboxamide | 541.09 |
| 448 | 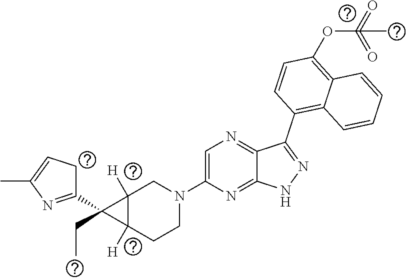 | 5-(6-((1S,6R,7S)-7-(aminomethyl)-7-(4- methylthiazol-2-yl)-3-azabicyclo[4.1.0]heptan-3-yl)- 2,4-dihydro-1H-pyrazolo[3,4-b]pyrazin-3- yl)quinolin-8-yl sulfurofluoridate | 566.63 |
| 449 | 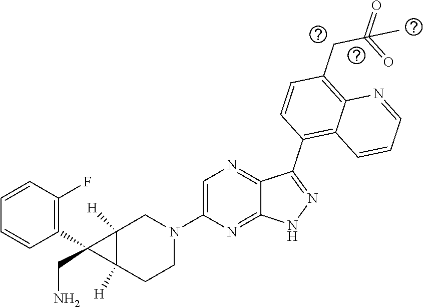 | 5-(6-((1S,6R,7R)-7-(aminomethyl)-7-(2- fluorophenyl)-3-azabicyclo[4.1.0]heptan-3-yl)-1,4- dihydro-712-pyrazolo[3,4-b]pyrazin-3-yl)quinolin-8- yl sulfurofluoridate | 563.58 |
| 450 | 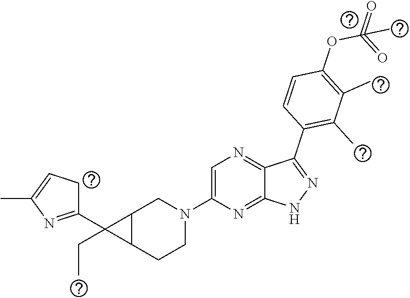 | 4-(6-(7-(aminomethyl)-7-(4-methyl-2H-114-thiazol-2- yl)-3-azabicyclo[4.1.0]heptan-3-yl)-1H-pyrazolo[3,4- b]pyrazin-3-yl)-5,6-dichlorocyclohexa-1,4-dien-1-yl sulfurofluoridate | 584.47 |
| 451 | 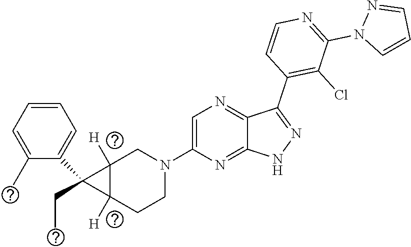 [SMILES: CC[C@]1(c2ccccc2C)C2CCN(c3cnc4c(-c5ccnc(-n6cccn6)c5Cl)n[nH]c4n3)CC21] | ((1S,6R,7R)-3-(3-(3-chloro-2-(1H-pyrazol-1- yl)pyridin-4-yl)-1H-pyrazolo[3,4-b]pyrazin-6-yl)-7- (2-fluorophenyl)-3-azabicyclo[4.1.0]heptan-7- yl)methanamine | 515.97 |
| 452 | 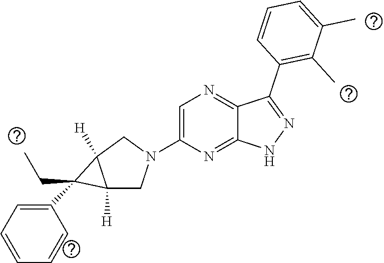 [SMILES: CC[C@]1(c2ccccc2)[C@@H]2CN(c3cnc4c(-c5cccc(C)c5C)n[nH]c4n3)C[C@@H]21] | ((1R,5S,6s)-3-(3-(2,3-dichlorophenyl)-1H- pyrazolo[3,4-b]pyrazin-6-yl)-6-phenyl-3- azabicyclo[3.1.0]hexan-6-yl)methanamine | 451.35 |
| 453 | 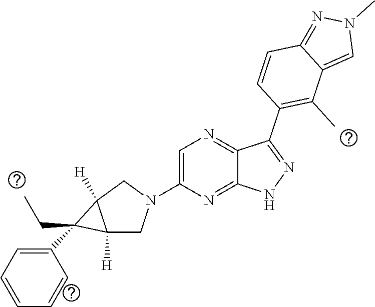 [SMILES: CC[C@]1(c2ccccc2)[C@@H]2CN(c3cnc4c(-c5ccc6nn(C)cc6c5C)n[nH]c4n3)C[C@@H]21] | ((1R,5S,6s)-3-(3-(4-chloro-2-methyl-2H-indazol-5- yl)-1H-pyrazolo[3,4-b]pyrazin-6-yl)-6-phenyl-3- azabicyclo[3.1.0]hexan-6-yl)methanamine | 470.96 |
| 454 | 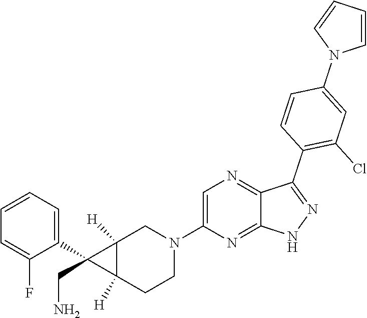 [SMILES: NC[C@]1(c2ccccc2F)[C@@H]2CCN(c3cnc4c(-c5ccc(-n6cccc6)cc5Cl)n[nH]c4n3)C[C@@H]21] | ((1S,6R,7R)-3-(3-(2-chloro-4-(2H-114-pyrrol-1- yl)phenyl)-1H-pyrazolo[3,4-b]pyrazin-6-yl)-7-(2- fluorophenyl)-3-azabicyclo[4.1.0]heptan-7- yl)methanamine | 514 |
| 455 | 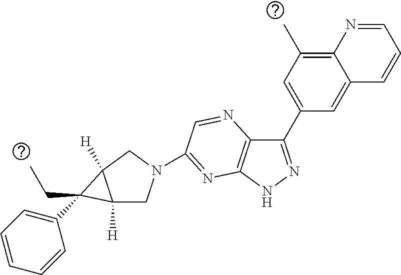 [SMILES: CC[C@]1(c2ccccc2)[C@@H]2CN(c3cnc4c(-c5cc(C)c6ncccc6c5)n[nH]c4n3)C[C@@H]21] | ((1R,5S,6s)-3-(3-(8-fluoroquinolin-6-yl)-1,7-dihydro- 412-pyrazolo[4,3-b]pyrazin-6-yl)-6-phenyl-3- azabicyclo[3.1.0]hexan-6-yl)methanamine | 451.5 |
| 456 | 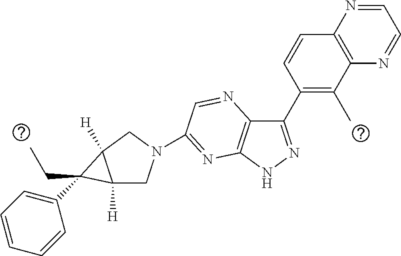 [SMILES: CC[C@]1(c2ccccc2)[C@@H]2CN(c3cnc4c(-c5ccc6nccnc6c5C)n[nH]c4n3)C[C@@H]21] | ((1R,5S,6s)-3-(3-(5-chloroquinoxalin-6-yl)-1H- pyrazolo[3,4-b]pyrazin-6-yl)-6-phenyl-3- azabicyclo[3.1.0]hexan-6-yl)methanamine | 468.94 |
| 457 | 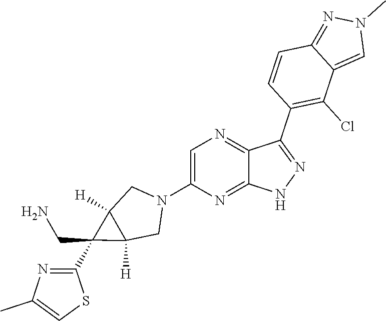 | ((1R,5S,6r)-3-(3-(4-chloro-2-methyl-2H-indazol-5- yl)-1H-pyrazolo[3,4-b]pyrazin-6-yl)-6-(4- methylthiazol-2-yl)-3-azabicyclo[3.1.0]hexan-6- yl)methanamine | 492 |
| 458 | 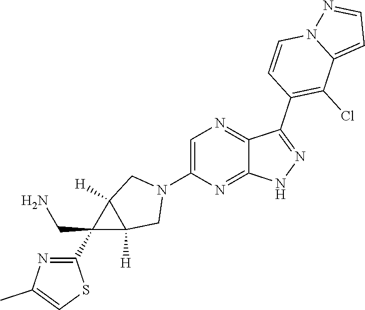 [SMILES: Cc1csc([C@@]2(CN)[C@@H]3CN(c4cnc5c(-c6ccn7nccc7c6Cl)n[nH]c5n4)C[C@@H]32)n1] | ((1R,5S,6r)-3-(3-(4-chloropyrazolo[1,5-a]pyridin-5- yl)-1H-pyrazolo[3,4-b]pyrazin-6-yl)-6-(4- methylthiazol-2-yl)-3-azabicyclo[3.1.0]hexan-6- yl)methanamine | 477.97 |
| 459 | 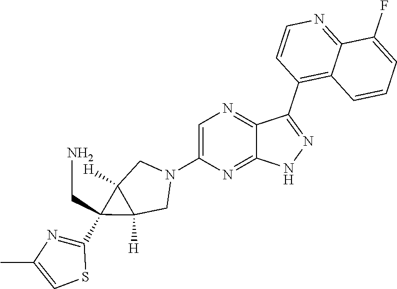 [SMILES: Cc1csc([C@@]2(CN)[C@@H]3CN(c4cnc5c(-c6ccnc7c(F)cccc67)n[nH]c5n4)C[C@@H]32)n1] | ((1R,5S,6r)-3-(3-(8-fluorooctahydro-2H-112-quinolin- 4-yl)-6,7-dihydro-1H-pyrazolo[3,4-b]pyrazin-6-yl)-6- (4-methylthiazol-2-yl)-3-azabicyclo[3.1.0]hexan-6- yl)methanamine | 472.54 |
| 460 | 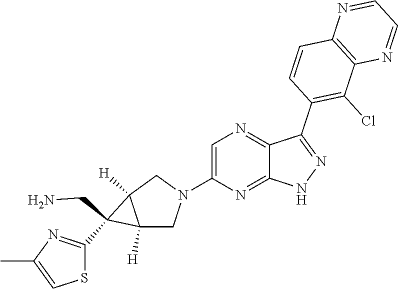 [SMILES: Cc1csc([C@@]2(CN)[C@@H]3CN(c4cnc5c(-c6ccc7nccnc7c6Cl)n[nH]c5n4)C[C@@H]32)n1] | ((1R,5S,6r)-3-(3-(5-chloroquinoxalin-6-yl)-1H- pyrazolo[3,4-b]pyrazin-6-yl)-6-(4-methylthiazol-2- yl)-3-azabicyclo[3.1.0]hexan-6-yl)methanamine | 489.98 |
| 461 | 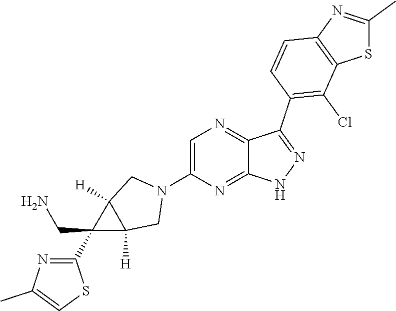 | ((1R,5S,6r)-3-(3-(7-chloro-2-methylbenzo[d]thiazol- 6-yl)-1,7-dihydro-412-pyrazolo[4,3-b]pyrazin-6-yl)-6- (4-methylthiazol-2-yl)-3-azabicyclo[3.1.0]hexan-6- yl)methanamine | 509.05 |
| 462 | 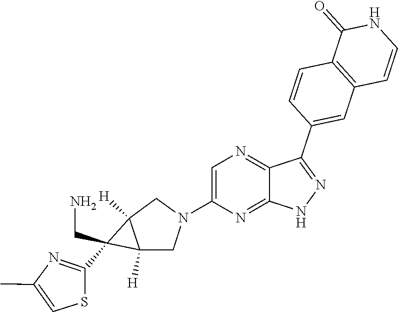 [SMILES: Cc1csc([C@@]2(CN)[C@@H]3CN(c4cnc5c(-c6ccc7c(=O)[nH]ccc7c6)n[nH]c5n4)C[C@@H]32)n1] | 6-(6-((1R,5S,6r)-6-(aminomethyl)-6-((S)-4-methyl- 2,3-dihydrothiazol-2-yl)-3-azabicyclo[3.1.0]hexan-3- yl)-3,7-dihydro-114,212-pyrazolo[3,4-b]pyrazin-3- yl)isoquinolin-1(2H)-one | 470.55 |
| 463 | 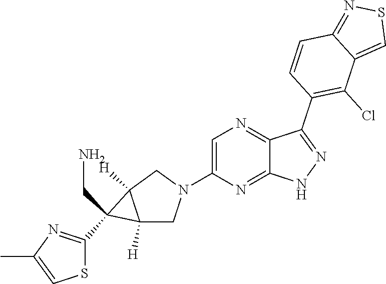 [SMILES: Cc1csc([C@@]2(CN)[C@@H]3CN(c4cnc5c(-c6ccc7nscc7c6Cl)n[nH]c5n4)C[C@@H]32)n1] | ((1R,5S,6r)-3-(3-(4-chlorobenzo[c]isothiazol-5-yl)- 1H-pyrazolo[3,4-b]pyrazin-6-yl)-6-(4-methylthiazol- 2-yl)-3-azabicyclo[3.1.0]hexan-6-yl)methanamine | 495.02 |
| 464 | 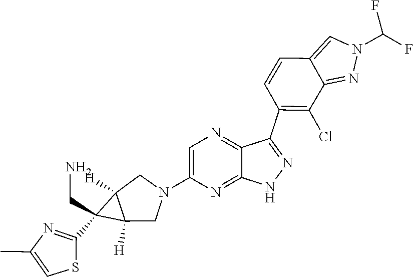 [SMILES: Cc1csc([C@@]2(CN)[C@@H]3CN(c4cnc5c(-c6ccc7cn(C(F)F)nc7c6Cl)n[nH]c5n4)C[C@@H]32)n1] | ((1R,5S,6r)-3-(3-(7-chloro-2-(difluoromethyl)-4H- 214-indazol-6-yl)-2,3-dihydro-1H-pyrazolo[3,4- b]pyrazin-6-yl)-6-((R)-4-methyl-312-thiazol-2-yl)-3- azabicyclo[3.1.0]hexan-6-yl)methanamine | 527.98 |
| 465 | 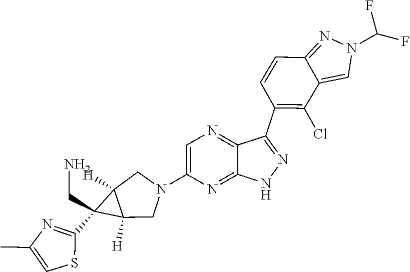 [SMILES: Cc1csc([C@@]2(CN)[C@@H]3CN(c4cnc5c(-c6ccc7nn(C(F)F)cc7c6Cl)n[nH]c5n4)C[C@@H]32)n1] | ((1R,5S,6r)-3-(3-(2-(difluoromethyl)-4-fluoro-2H- indazol-5-yl)-1H-pyrazolo[3,4-b]pyrazin-6-yl)-6-(4- methylthiazol-2-yl)-3-azabicyclo[3.1.0]hexan-6- yl)methanamine | 511.53 |
| 466 | 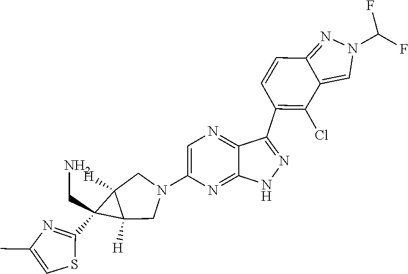 [SMILES: Cc1csc([C@@]2(CN)[C@@H]3CN(c4cnc5c(-c6ccc7nn(C(F)F)cc7c6Cl)n[nH]c5n4)C[C@@H]32)n1] | ((1R,5S,6r)-3-(3-(4-chloro-2-(difluoromethyl)-2H- indazol-5-yl)-1H-pyrazolo[3,4-b]pyrazin-6-yl)-6-(4- methylthiazol-2-yl)-3-azabicyclo[3.1.0]hexan-6- yl)methanamine | 527.98 |
| 467 | 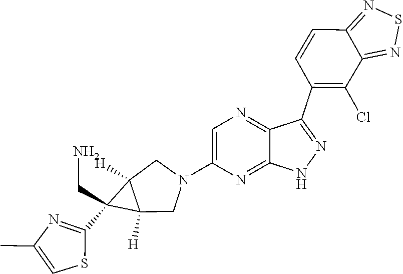 [SMILES: Cc1csc([C@@]2(CN)[C@@H]3CN(c4cnc5c(-c6ccc7nsnc7c6Cl)n[nH]c5n4)C[C@@H]32)n1] | ((1R,5S,6r)-3-(3-(4-chlorobenzo[c][1,2,5]thiadiazol- 5-yl)-1H-pyrazolo[3,4-b]pyrazin-6-yl)-6-(4- methylthiazol-2-yl)-3-azabicyclo[3.1.0]hexan-6- yl)methanamine | 496.01 |
| 468 | 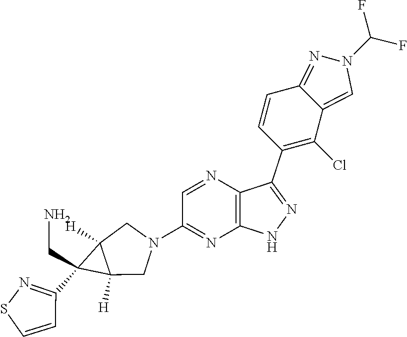 [SMILES: NC[C@]1(c2ccsn2)[C@@H]2CN(c3cnc4c(-c5ccc6nn(C(F)F)cc6c5Cl)n[nH]c4n3)C[C@@H]21] | ((1R,5S,6r)-3-(3-(4-chloro-2-(difluoromethyl)-4,7- dihydro-2H-indazol-5-yl)-1H-pyrazolo[3,4- b]pyrazin-6-yl)-6-(isothiazol-3-yl)-3- azabicyclo[3.1.0]hexan-6-yl)methanamine | 513.95 |
| 469 | 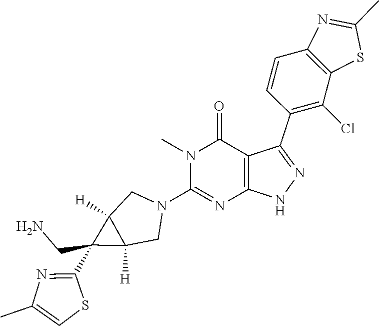 | 6-((1R,5S,6r)-6-(aminomethyl)-6-(4-methylthiazol-2- yl)-3-azabicyclo[3.1.0]hexan-3-yl)-3-(7-chloro-2- methylbenzo[d]thiazol-6-yl)-5-methyl-1,5-dihydro- 4H-pyrazolo[3,4-d]pyrimidin-4-one | 539.08 |
| 470 | 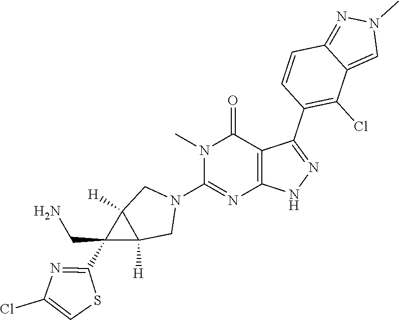 [SMILES: Cn1cc2c(Cl)c(-c3n[nH]c4nc(N5C[C@@H]6[C@H](C5)[C@@]6(CN)c5nc(Cl)cs5)n(C)c(=O)c34)ccc2n1] | 6-((1R,5S,6r)-6-(aminomethyl)-6-(4-chlorothiazol-2- yl)-3-azabicyclo[3.1.0]hexan-3-yl)-3-(4-chloro-2- methyl-2H-indazol-5-yl)-5-methyl-1,5-dihydro-4H- pyrazolo[3,4-d]pyrimidin-4-one | 542.44 |
| 471 | 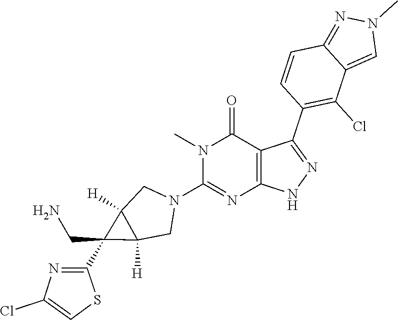 [SMILES: Cn1cc2c(Cl)c(-c3n[nH]c4nc(N5C[C@@H]6[C@H](C5)[C@@]6(CN)c5nc(Cl)cs5)n(C)c(=O)c34)ccc2n1] | 6-((1R,5S,6r)-6-(aminomethyl)-6-(4- (trifluoromethyl)thiazol-2-yl)-3- azabicyclo[3.1.0]hexan-3-yl)-3-(4-chloro-2-methyl- 2H-indazol-5-yl)-5-methyl-1,5-dihydro-4H- pyrazolo[3,4-d]pyrimidin-4-one | 576 |
| 472 | 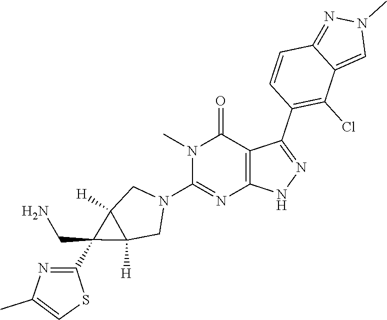 | 6-((1R,5S,6r)-6-(aminomethyl)-6-(4-methylthiazol-2- yl)-3-azabicyclo[3.1.0]hexan-3-yl)-3-(4-chloro-2- methyl-2H-indazol-5-yl)-5-methyl-1,5-dihydro-4H- pyrazolo[3,4-d]pyrimidin-4-one | 522.02 |
| 473 | 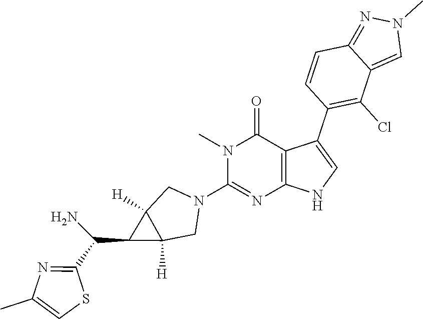 | 2-((1R,5S,6r)-6-(aminomethyl)-6-(4-methylthiazol-2- yl)-3-azabicyclo[3.1.0]hexan-3-yl)-5-(4-chloro-2- methyl-2H-indazol-5-yl)-3-methyl-3,7-dihydro-4H- pyrrolo[2,3-d]pyrimidin-4-one | 521.04 |
| 474 | 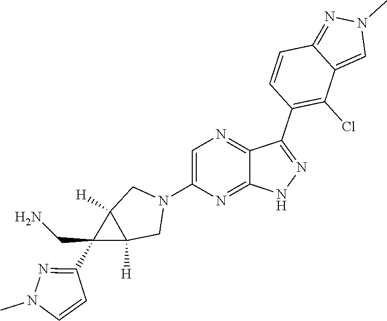 | ((1R,5S,6r)-3-(3-(4-chloro-2-methyl-2H-indazol-5- yl)-1H-pyrazolo[3,4-b]pyrazin-6-yl)-6-(1-methyl-1H- pyrazol-3-yl)-3-azabicyclo[3.1.0]hexan-6- yl)methanamine | 474.95 |
| 475 | 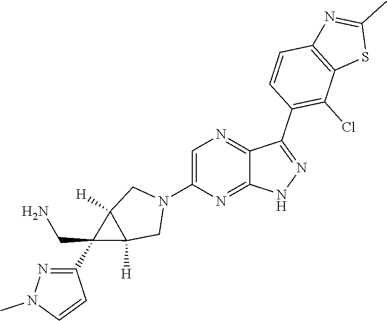 | ((1R,5S,6r)-3-(3-(7-chloro-2-methylbenzo[d]thiazol- 6-yl)-1H-pyrazolo[3,4-b]pyrazin-6-yl)-6-(1-methyl- 1H-pyrazol-3-yl)-3-azabicyclo[3.1.0]hexan-6- yl)methanamine | 492 |
| 476 | 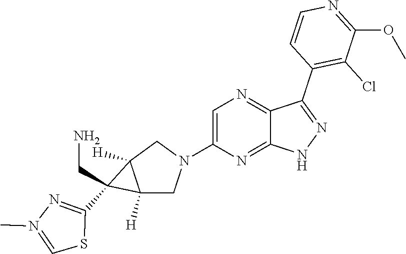 | ((1R,5S,6r)-3-(3-(3-chloro-2-methoxypyridin-4-yl)- 1H-pyrazolo[3,4-b]pyrazin-6-yl)-6-(4-methylthiazol- 2-yl)-3-azabicyclo[3.1.0]hexan-6-yl)methanamine | 468.96 |
| 477 | 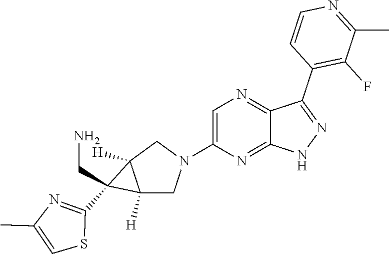 | ((1R,5S,6r)-3-(3-(3-fluoro-2-methyl-1,2- dihydropyridin-4-yl)-1H-pyrazolo[3,4-b]pyrazin-6- yl)-6-(4-methylthiazol-2-yl)-3- azabicyclo[3.1.0]hexan-6-yl)methanamine | 436.51 |
| 478 | 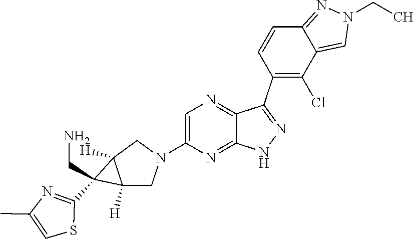 [SMILES: CCn1cc2c(Cl)c(-c3n[nH]c4nc(N5C[C@@H]6[C@H](C5)[C@@]6(CN)c5nc(C)cs5)cnc34)ccc2n1] | 2-(5-(6-((1R,5S,6r)-6-(aminomethyl)-6-(4- methylthiazol-2-yl)-3-azabicyclo[3.1.0]hexan-3-yl)- 1H-pyrazolo[3,4-b]pyrazin-3-yl)-4-chloro-2H- indazol-2-yl)acetonitrile | 517.01 |
| 479 | 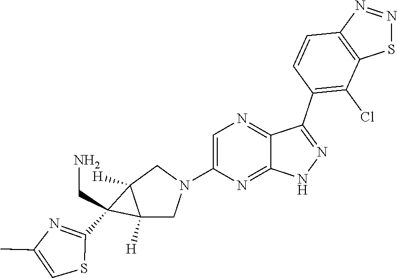 [SMILES: Cc1csc([C@@]2(CN)[C@@H]3CN(c4cnc5c(-c6ccc7nnsc7c6Cl)n[nH]c5n4)C[C@@H]32)n1] | ((1R,5S,6r)-3-(3-(7-chlorobenzo[d][1,2,3]thiadiazol- 6-yl)-1H-pyrazolo[3,4-b]pyrazin-6-yl)-6-(4- methylthiazol-2-yl)-3-azabicyclo[3.1.0]hexan-6- yl)methanamine | 496.01 |
| 480 | 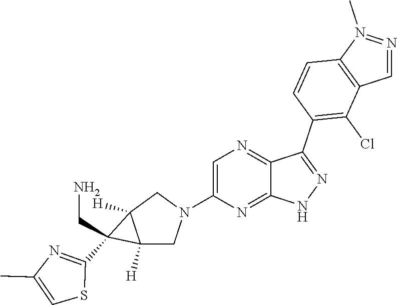 | ((1R,5S,6r)-3-(3-(4-chloro-1-methyl-1H-indazol-5- yl)-1H-pyrazolo[3,4-b]pyrazin-6-yl)-6-(4- methylthiazol-2-yl)-3-azabicyclo[3.1.0]hexan-6- yl)methanamine | 492 |
| 481 | 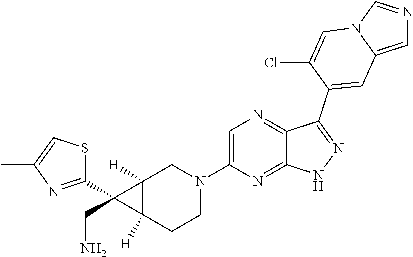 [SMILES: Cc1csc([C@@]2(CN)[C@@H]3CCN(c4cnc5c(-c6cc7cncn7cc6Cl)n[nH]c5n4)C[C@@H]32)n1] | ((1S,6R,7S)-3-(3-(6-chloropyrazolo[1,5-a]pyridin-5- yl)-1H-pyrazolo[3,4-b]pyrazin-6-yl)-7-(4- methylthiazol-2-yl)-3-azabicyclo[4.1.0]heptan-7- yl)methanamine | 492 |
| 482 | 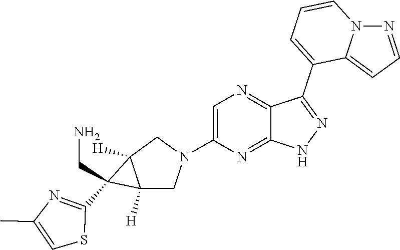 [SMILES: Cc1csc([C@@]2(CN)[C@@H]3CN(c4cnc5c(-c6cccn7nccc67)n[nH]c5n4)C[C@@H]32)n1] | ((1R,5S,6r)-6-(4-methylthiazol-2-yl)-3-(3- (pyrazolo[1,5-a]pyridin-4-yl)-1H-pyrazolo[3,4- b]pyrazin-6-yl)-3-azabicyclo[3.1.0]hexan-6- yl)methanamine | 443.53 |
| 483 | 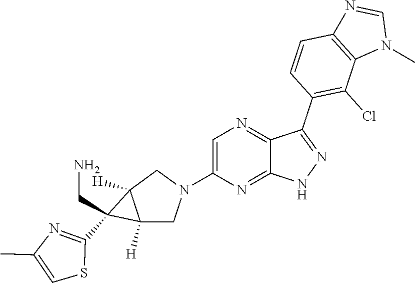 | ((1R,5S,6r)-3-(3-(7-chloro-1-methyl-1H- benzo[d]imidazol-6-yl)-1H-pyrazolo[3,4-b]pyrazin-6- yl)-6-(4-methylthiazol-2-yl)-3- azabicyclo[3.1.0]hexan-6-yl)methanamine | 492 |
| 484 | 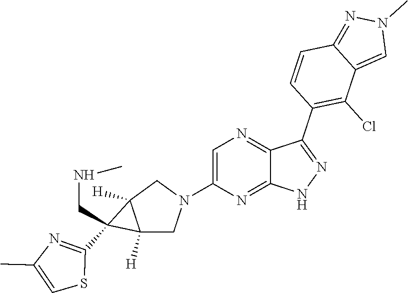 | 1-((1R,5S,6r)-3-(3-(4-chloro-2-methyl-2H-indazol-5- yl)-1H-pyrazolo[3,4-b]pyrazin-6-yl)-6-(4- methylthiazol-2-yl)-3-azabicyclo[3.1.0]hexan-6-yl)- N-methylmethanamine | 506.03 |
| 485 | 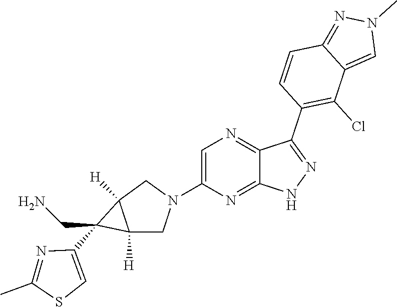 | ((1R,5S,6r)-3-(3-(4-chloro-2-methyl-2H-indazol-5- yl)-1H-pyrazolo[3,4-b]pyrazin-6-yl)-6-(2- methylthiazol-4-yl)-3-azabicyclo[3.1.0]hexan-6- yl)methanamine | 492 |
| 486 | 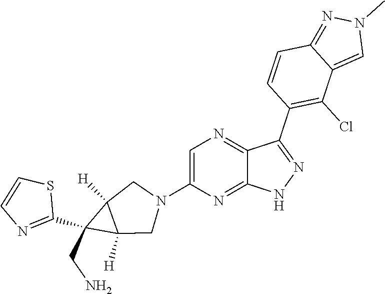 [SMILES: Cn1cc2c(Cl)c(-c3n[nH]c4nc(N5C[C@@H]6[C@H](C5)[C@@]6(CN)c5nccs5)cnc34)ccc2n1] | ((1R,5S,6r)-3-(3-(4-chloro-2-methyl-2H-indazol-5- yl)-1H-pyrazolo[3,4-b]pyrazin-6-yl)-6-(thiazol-2-yl)- 3-azabicyclo[3.1.0]hexan-6-yl)methanamine | 477.97 |
| 487 | 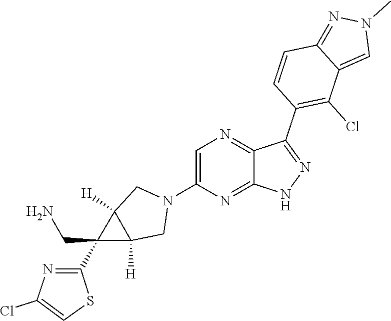 [SMILES: Cn1cc2c(Cl)c(-c3n[nH]c4nc(N5C[C@@H]6[C@H](C5)[C@@]6(CN)c5nc(Cl)cs5)cnc34)ccc2n1] | ((1R,5S,6r)-3-(3-(4-chloro-2-methyl-2H-indazol-5- yl)-1H-pyrazolo[3,4-b]pyrazin-6-yl)-6-(4- chlorothiazol-2-yl)-3-azabicyclo[3.1.0]hexan-6- yl)methanamine | 512.42 |
| 488 | 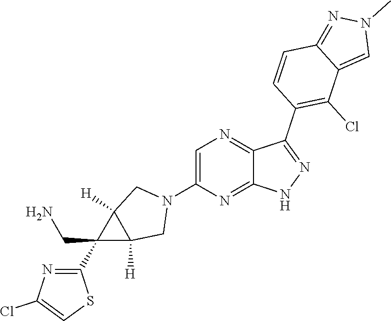 [SMILES: Cn1cc2c(Cl)c(-c3n[nH]c4nc(N5C[C@@H]6[C@H](C5)[C@@]6(CN)c5nc(Cl)cs5)cnc34)ccc2n1] | ((1R,5S,6r)-3-(3-(4-chloro-2-methyl-2H-indazol-5- yl)-1H-pyrazolo[3,4-b]pyrazin-6-yl)-6-(4- (trifluoromethyl)thiazol-2-yl)-3- azabicyclo[3.1.0]hexan-6-yl)methanamine | 545.97 |
| 489 | 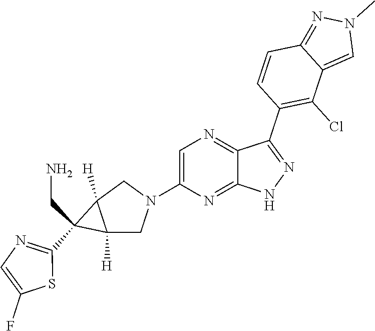 [SMILES: Cn1cc2c(Cl)c(-c3n[nH]c4nc(N5C[C@@H]6[C@H](C5)[C@@]6(CN)c5ncc(F)s5)cnc34)ccc2n1] | ((1R,5S,6r)-3-(3-(4-chloro-2-methyl-2H-indazol-5- yl)-1H-pyrazolo[3,4-b]pyrazin-6-yl)-6-(5- fluorothiazol-2-yl)-3-azabicyclo[3.1.0]hexan-6- yl)methanamine | 495.96 |
| 490 | 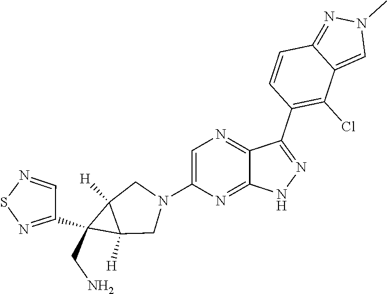 [SMILES: Cn1cc2c(Cl)c(-c3n[nH]c4nc(N5C[C@@H]6[C@H](C5)[C@@]6(CN)c5cnsn5)cnc34)ccc2n1] | ((1R,5S,6r)-3-(3-(4-chloro-2-methyl-2H-indazol-5- yl)-1H-pyrazolo[3,4-b]pyrazin-6-yl)-6-(1,2,5- thiadiazol-3-yl)-3-azabicyclo[3.1.0]hexan-6- yl)methanamine | 478.96 |
| 491 | 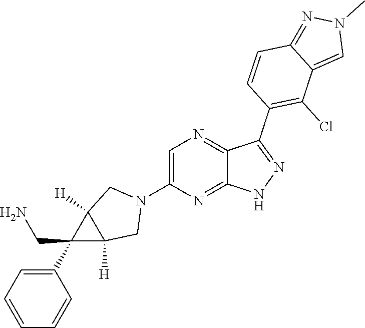 [SMILES: Cn1cc2c(Cl)c(-c3n[nH]c4nc(N5C[C@@H]6[C@H](C5)[C@@]6(CN)c5ccccc5)cnc34)ccc2n1] | ((1R,5S,6r)-3-(3-(4-chloro-2-methyl-2H-indazol-5- yl)-1H-pyrazolo[3,4-b]pyrazin-6-yl)-6-(pyridin-2-yl)- 3-azabicyclo[3.1.0]hexan-6-yl)methanamine | 471.94 |
| 492 | 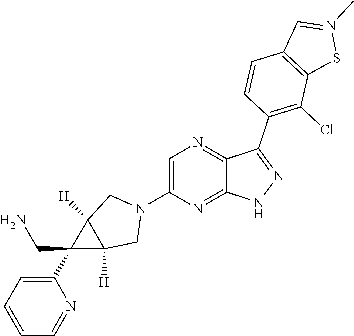 | ((1R,5S,6r)-3-(3-(7-chloro-2-methylbenzo[d]thiazol- 6-yl)-1H-pyrazolo[3,4-b]pyrazin-6-yl)-6-(pyridin-2- yl)-3-azabicyclo[3.1.0]hexan-6-yl)methanamine | 489 |
| 493 | 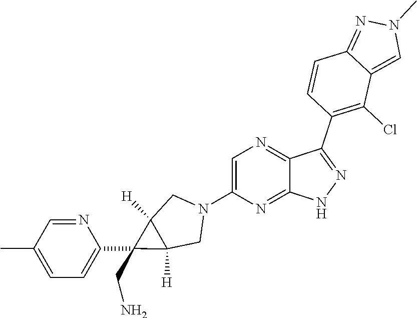 | ((1R,5S,6r)-3-(3-(4-chloro-2-methyl-3H-214-indazol- 5-yl)-1H-pyrazolo[3,4-b]pyrazin-6-yl)-6-(5-methyl- 1,4-dihydropyridin-2-yl)-3-azabicyclo[3.1.0]hexan-6- yl)methanamine | 485.97 |
| 494 | 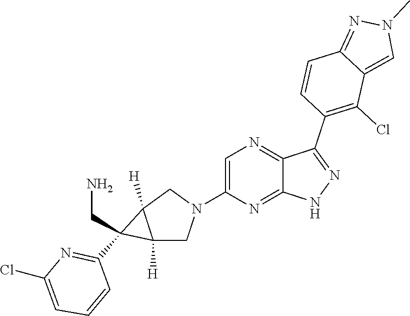 [SMILES: Cn1cc2c(Cl)c(-c3n[nH]c4nc(N5C[C@@H]6[C@H](C5)[C@@]6(CN)c5cccc(Cl)n5)cnc34)ccc2n1] | ((1R,5S,6r)-3-(3-(4-chloro-2-methyl-2H-indazol-5- yl)-1H-pyrazolo[3,4-b]pyrazin-6-yl)-6-(6- chloropyridin-2-yl)-3-azabicyclo[3.1.0]hexan-6- yl)methanamine | 506.39 |
| 495 | 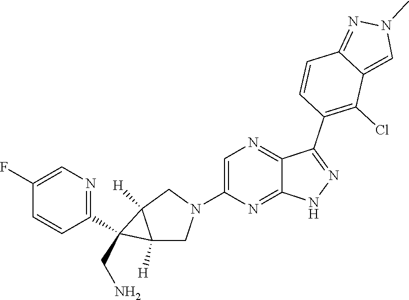 [SMILES: Cn1cc2c(Cl)c(-c3n[nH]c4nc(N5C[C@@H]6[C@H](C5)[C@@]6(CN)c5ccc(F)cn5)cnc34)ccc2n1] | ((1R,5S,6r)-3-(3-(4-chloro-2-methyl-2H-indazol-5- yl)-1H-pyrazolo[3,4-b]pyrazin-6-yl)-6-(5- fluoropyridin-2-yl)-3-azabicyclo[3.1.0]hexan-6- yl)methanamine | 489.94 |
| 496 | 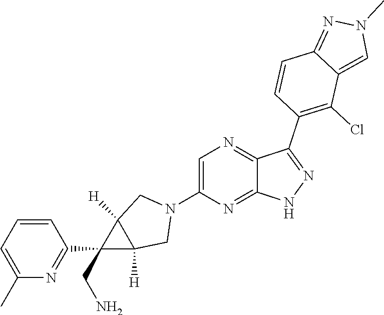 [SMILES: Cc1cccc([C@@]2(CN)[C@@H]3CN(c4cnc5c(-c6ccc7nn(C)cc7c6Cl)n[nH]c5n4)C[C@@H]32)n1] | ((1R,5S,6r)-3-(3-(4-chloro-2-methyl-3,7-dihydro-2H- indazol-5-yl)-1H-pyrazolo[3,4-b]pyrazin-6-yl)-6-(6- methyl-1,6-dihydropyridin-2-yl)-3- azabicyclo[3.1.0]hexan-6-yl)methanamine | 485.97 |
| 497 | 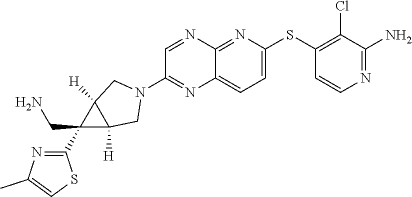 [SMILES: Cc1csc([C@@]2(CN)[C@@H]3CN(c4cnc5nc(Sc6ccnc(N)c6Cl)ccc5n4)C[C@@H]32)n1] | 4-((2-((1R,5S,6r)-6-(aminomethyl)-6-(4- methylthiazol-2-yl)-3-azabicyclo[3.1.0]hexan-3- yl)pyrido[2,3-b]pyrazin-6-yl)thio)-3-chloropyridin-2- amine | 497.04 |
| 498 | 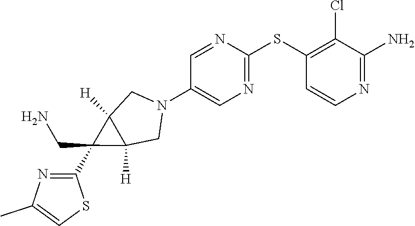 [SMILES: Cc1csc([C@@]2(CN)[C@@H]3CN(c4cnc(Sc5ccnc(N)c5Cl)nc4)C[C@@H]32)n1] | 4-((5-((1R,5S,6r)-6-(aminomethyl)-6-(4- methylthiazol-2-yl)-3-azabicyclo[3.1.0]hexan-3-yl)- 1,4-dihydropyrazin-2-yl)thio)-3-chloropyridin-2- amine | 445.99 |
| 499 | 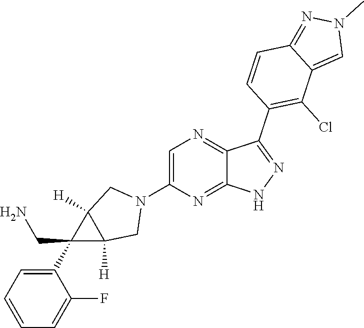 [SMILES: Cn1cc2c(Cl)c(-c3n[nH]c4nc(N5C[C@@H]6[C@H](C5)[C@@]6(CN)c5ccccc5F)cnc34)ccc2n1] | ((1R,5S,6s)-3-(3-(4-chloro-2-methyl-2H-indazol-5- yl)-1H-pyrazolo[3,4-b]pyrazin-6-yl)-6-(2- fluorophenyl)-3-azabicyclo[3.1.0]hexan-6- yl)methanamine | 488.95 |
| 500 | 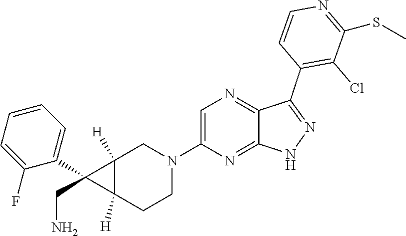 [SMILES: CSc1nccc(-c2n[nH]c3nc(N4CC[C@@H]5[C@H](C4)[C@@]5(CN)c4ccccc4F)cnc23)c1Cl] | ((1S,6R,7R)-3-(3-(3-chloro-2-methoxypyridin-4-yl)- 1H-pyrazolo[3,4-b]pyrazin-6-yl)-7-(2-fluorophenyl)- 3-azabicyclo[4.1.0]heptan-7-yl)methanamine | 479.94 |
| 501 | 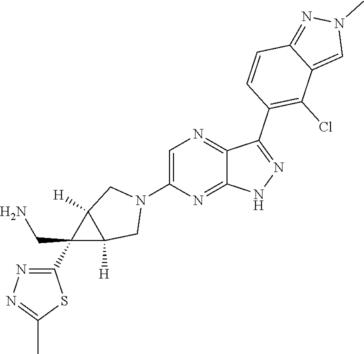 [SMILES: Cc1nnc([C@@]2(CN)[C@@H]3CN(c4cnc5c(-c6ccc7nn(C)cc7c6Cl)n[nH]c5n4)C[C@@H]32)s1] | ((1R,5S,6r)-3-(3-(4-chloro-2-methyl-2H-indazol-5- yl)-1H-pyrazolo[3,4-b]pyrazin-6-yl)-6-(5-methyl- 1,3,4-thiadiazol-2-yl)-3-azabicyclo[3.1.0]hexan-6- yl)methanamine | 492.99 |
| 502 | 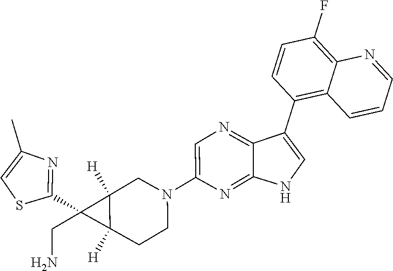 [SMILES: Cc1csc([C@@]2(CN)[C@@H]3CCN(c4cnc5c(-c6ccc(F)c7ncccc67)c[nH]c5n4)C[C@@H]32)n1] | ((1S,6R,7S)-3-(7-(8-fluoroquinolin-5-yl)-5H- pyrrolo[2,3-b]pyrazin-3-yl)-7-((R)-4-methyl-2,3- dihydrothiazol-2-yl)-3-azabicyclo[4.1.0]heptan-7- yl)methanamine | 485.58 |
| 503 | 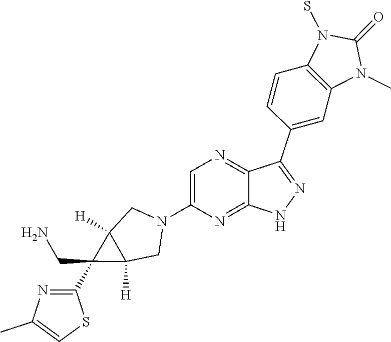 | 5-(6-((1R,5S,6r)-6-(aminomethyl)-6-(4- methylthiazol-2-yl)-3-azabicyclo[3.1.0]hexan-3-yl)- 1H-pyrazolo[3,4-b]pyrazin-3-yl)-1,3-dimethyl- 1,3,4,7-tetrahydro-2H-benzo[d]imidazol-2-one | 487.58 |
| 504 | 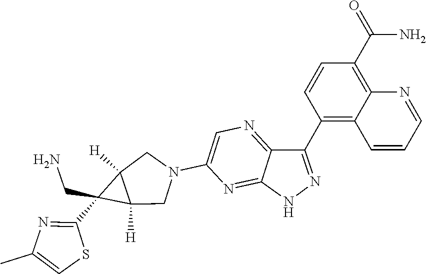 [SMILES: Cc1csc([C@@]2(CN)[C@@H]3CN(c4cnc5c(-c6ccc(C(N)=O)c7ncccc67)n[nH]c5n4)C[C@@H]32)n1] | 5-(6-((1R,5S,6r)-6-(aminomethyl)-6-(4- methylthiazol-2-yl)-3-azabicyclo[3.1.0]hexan-3-yl)- 1H-pyrazolo[3,4-b]pyrazin-3-yl)quinoline-8- carboxamide | 497.57 |
| 505 | 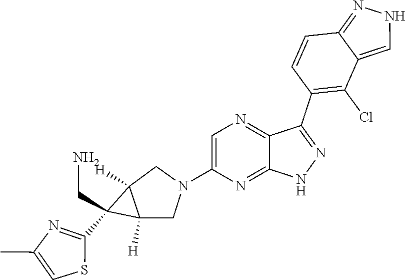 [SMILES: Cc1csc([C@@]2(CN)[C@@H]3CN(c4cnc5c(-c6ccc7n[nH]cc7c6Cl)n[nH]c5n4)C[C@@H]32)n1] | ((1R,5S,6r)-3-(3-(4-chloro-2H-indazol-5-yl)-1H- pyrazolo[3,4-b]pyrazin-6-yl)-6-(4-methylthiazol-2- yl)-3-azabicyclo[3.1.0]hexan-6-yl)methanamine | 477.97 |
| 506 | 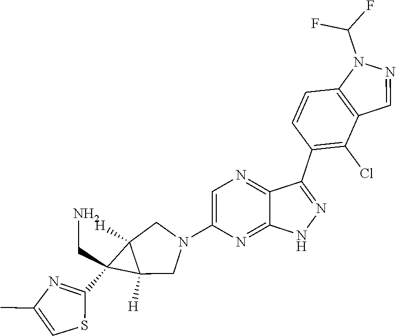 [SMILES: Cc1csc([C@@]2(CN)[C@@H]3CN(c4cnc5c(-c6ccc7c(cnn7C(F)F)c6Cl)n[nH]c5n4)C[C@@H]32)n1] | ((1R,5S,6r)-3-(3-(4-chloro-1-(difluoromethyl)-1H- indazol-5-yl)-1H-pyrazolo[3,4-b]pyrazin-6-yl)-6-(4- methylthiazol-2-yl)-3-azabicyclo[3.1.0]hexan-6- yl)methanamine | 527.98 |
| 507 | 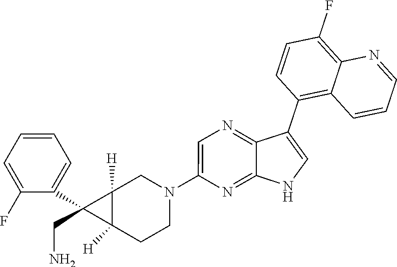 [SMILES: NC[C@]1(c2ccccc2F)[C@@H]2CCN(c3cnc4c(-c5ccc(F)c6ncccc56)c[nH]c4n3)C[C@@H]21] | ((1S,6R,7R)-7-(2-fluorophenyl)-3-(7-(8- fluoroquinolin-5-yl)-5H-pyrrolo[2,3-b]pyrazin-3-yl)- 3-azabicyclo[4.1.0]heptan-7-yl)methanamine | 482.53 |
| 508 | 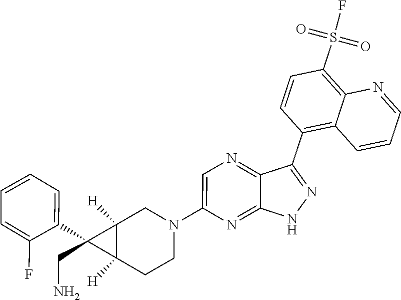 [SMILES: NC[C@]1(c2ccccc2F)[C@@H]2CCN(c3cnc4c(-c5ccc(S(=O)(=O)F)c6ncccc56)n[nH]c4n3)C[C@@H]21] | 5-(6-((1S,6R,7R)-7-(aminomethyl)-7-(2- fluorophenyl)-3-azabicyclo[4.1.0]heptan-3-yl)-4,7- dihydro-1H-pyrazolo[3,4-b]pyrazin-3-yl)quinoline-8- sulfonyl fluoride | 547.58 |
| 509 | 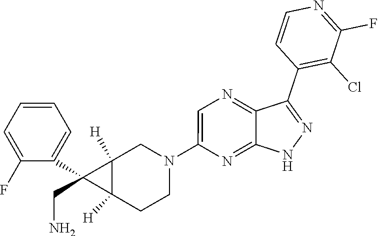 [SMILES: NC[C@]1(c2ccccc2F)[C@@H]2CCN(c3cnc4c(-c5ccnc(F)c5Cl)n[nH]c4n3)C[C@@H]21] | ((1S,6R,7R)-3-(3-(3-chloro-2-fluoropyridin-4-yl)-1H- pyrazolo[3,4-b]pyrazin-6-yl)-7-(2-fluorophenyl)-3- azabicyclo[4.1.0]heptan-7-yl)methanamine | 467.9 |
| 510 | 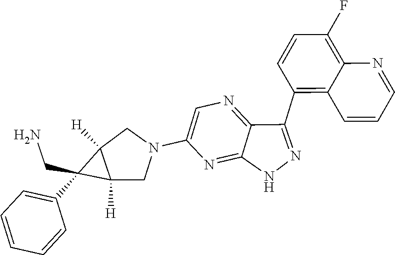 [SMILES: NC[C@]1(c2ccccc2)[C@@H]2CN(c3cnc4c(-c5ccc(F)c6ncccc56)n[nH]c4n3)C[C@@H]21] | ((1R,5S,6s)-3-(3-(8-fluoroquinolin-5-yl)-1H- pyrazolo[3,4-b]pyrazin-6-yl)-6-phenyl-3- azabicyclo[3.1.0]hexan-6-yl)methanamine | 451.5 |
| 511 | 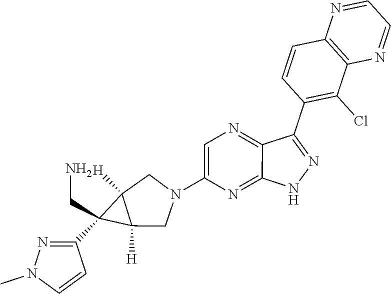 [SMILES: Cn1ccc([C@@]2(CN)[C@@H]3CN(c4cnc5c(-c6ccc7nccnc7c6Cl)n[nH]c5n4)C[C@@H]32)n1] | ((1R,5S,6r)-3-(3-(5-chloroquinoxalin-6-yl)-1H- pyrazolo[3,4-b]pyrazin-6-yl)-6-(1-methyl-1H- pyrazol-3-yl)-3-azabicyclo[3.1.0]hexan-6- yl)methanamine | 472.93 |
| 512 | 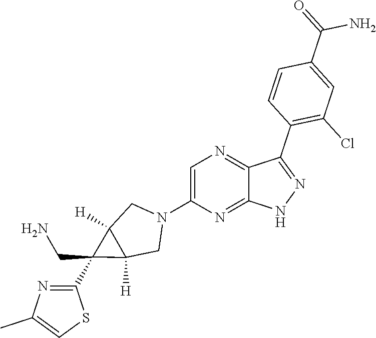 [SMILES: Cc1csc([C@@]2(CN)[C@@H]3CN(c4cnc5c(-c6ccc(C(N)=O)cc6Cl)n[nH]c5n4)C[C@@H]32)n1] | 4-(6-((1R,5S,6r)-6-(aminomethyl)-6-(4- methylthiazol-2-yl)-3-azabicyclo[3.1.0]hexan-3-yl)- 1H-pyrazolo[3,4-b]pyrazin-3-yl)-3-chlorobenzamide | 480.97 |
| 513 | 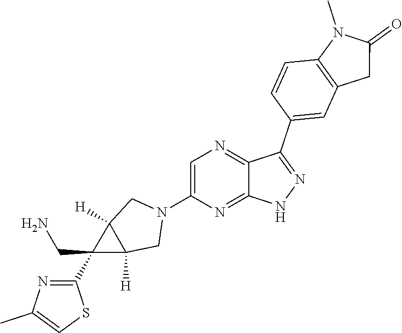 [SMILES: Cc1csc([C@@]2(CN)[C@@H]3CN(c4cnc5c(-c6ccc7c(c6)CC(=O)N7C)n[nH]c5n4)C[C@@H]32)n1] | 5-(6-((1R,5S,6r)-6-(aminomethyl)-6-((R)-4-methyl- 2,3-dihydrothiazol-2-yl)-3-azabicyclo[3.1.0]hexan-3- yl)-1H-pyrazolo[3,4-b]pyrazin-3-yl)-1-methylindolin- 2-one | 472.56 |
| 514 | 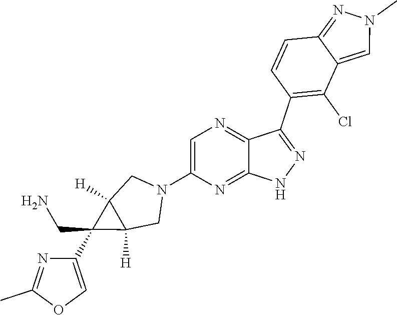 | ((1R,5S,6r)-3-(3-(4-chloro-2-methyl-3H-214-indazol- 5-yl)-1H-pyrazolo[3,4-b]pyrazin-6-yl)-6-(2- methyloxazol-4-yl)-3-azabicyclo[3.1.0]hexan-6- yl)methanamine | 475.93 |
| 515 | 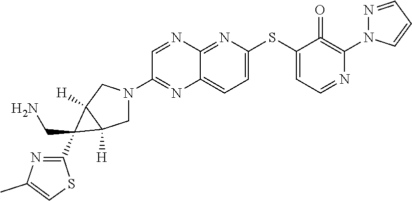 | ((1R,5S,6r)-3-(6-((3-chloro-2-(1H-pyrazol-1- yl)pyridin-4-yl)thio)pyrido[2,3-b]pyrazin-2-yl)-6-(4- methylthiazol-2-yl)-3-azabicyclo[3.1.0]hexan-6- yl)methanamine | 548.09 |
| 516 | 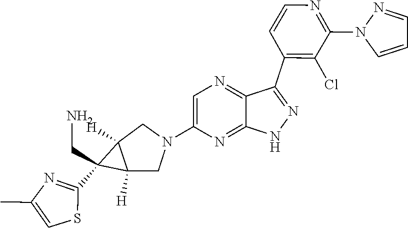 [SMILES: Cc1csc([C@@]2(CN)[C@@H]3CN(c4cnc5c(-c6ccnc(-n7cccn7)c6Cl)n[nH]c5n4)C[C@@H]32)n1] | ((1R,5S,6r)-3-(3-(3-chloro-2-(1H-pyrazol-1- yl)pyridin-4-yl)-1H-pyrazolo[3,4-b]pyrazin-6-yl)-6- (4-methylthiazol-2-yl)-3-azabicyclo[3.1.0]hexan-6- yl)methanamine | 505 |
| 517 | 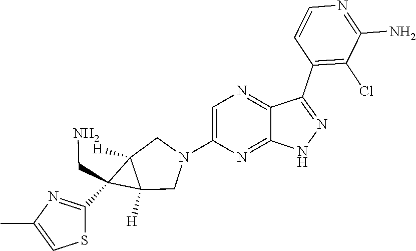 [SMILES: Cc1csc([C@@]2(CN)[C@@H]3CN(c4cnc5c(-c6ccnc(N)c6Cl)n[nH]c5n4)C[C@@H]32)n1] | 4-(6-((1R,5S,6r)-6-(aminomethyl)-6-(4- methylthiazol-2-yl)-3-azabicyclo[3.1.0]hexan-3-yl)- 1H-pyrazolo[3,4-b]pyrazin-3-yl)-3-chloropyridin-2- amine | 453.95 |
| 518 | 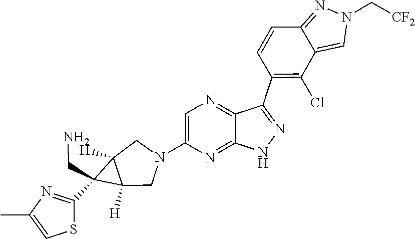 | ((1R,5S,6r)-3-(3-(4-chloro-2-(2,2,2-trifluoroethyl)- 2H-indazol-5-yl)-1,7-dihydro-412-pyrazolo[4,3- b]pyrazin-6-yl)-6-(4-methylthiazol-2-yl)-3- azabicyclo[3.1.0]hexan-6-yl)methanamine | 560 |
| 519 | 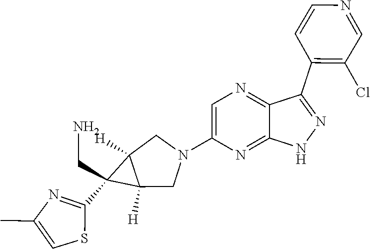 [SMILES: Cc1csc([C@@]2(CN)[C@@H]3CN(c4cnc5c(-c6ccncc6Cl)n[nH]c5n4)C[C@@H]32)n1] | ((1R,5S,6r)-3-(3-(3-chloropyridin-4-yl)-1H- pyrazolo[3,4-b]pyrazin-6-yl)-6-(4-methylthiazol-2- yl)-3-azabicyclo[3.1.0]hexan-6-yl)methanamine | 438.94 |
| 520 | 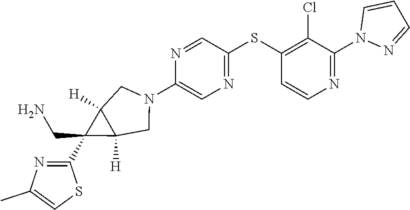 [SMILES: Cc1csc([C@@]2(CN)[C@@H]3CN(c4cnc(Sc5ccnc(-n6cccn6)c5Cl)cn4)C[C@@H]32)n1] | ((1R,5S,6r)-3-(5-((3-chloro-2-(1H-pyrazol-1-yl)-1,4- dihydropyridin-4-yl)thio)-1,2-dihydropyrazin-2-yl)-6- (4-methylthiazol-2-yl)-3-azabicyclo[3.1.0]hexan-6- yl)methanamine | 497.04 |
| 521 | 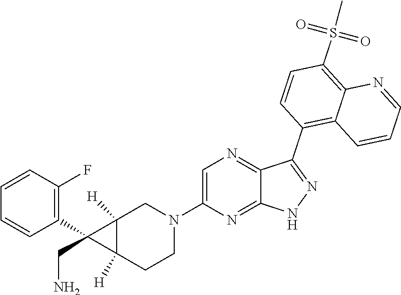 [SMILES: CS(=O)(=O)c1ccc(-c2n[nH]c3nc(N4CC[C@@H]5[C@H](C4)[C@@]5(CN)c4ccccc4F)cnc23)c2cccnc12] | ((1S,6R,7R)-7-(2-fluorophenyl)-3-(3-(8- (methylsulfonyl)quinolin-5-yl)-1H-pyrazolo[3,4- b]pyrazin-6-yl)-3-azabicyclo[4.1.0]heptan-7- yl)methanamine | 543.62 |
| 522 | 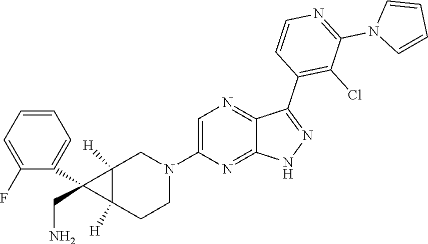 [SMILES: NC[C@]1(c2ccccc2F)[C@@H]2CCN(c3cnc4c(-c5ccnc(-n6cccc6)c5Cl)n[nH]c4n3)C[C@@H]21] | ((1S,6R,7R)-3-(3-(5-chloro-6-(1H-pyrrol-1-yl)-1,2- dihydropyridin-4-yl)-1H-pyrazolo[3,4-b]pyrazin-6- yl)-7-(2-fluorophenyl)-3-azabicyclo[4.1.0]heptan-7- yl)methanamine | 514.98 |
| 523 | 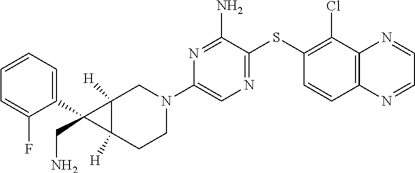 [SMILES: NC[C@]1(c2ccccc2F)[C@@H]2CCN(c3cnc(Sc4ccc5nccnc5c4Cl)c(N)n3)C[C@@H]21] | 6-((1S,6R,7R)-7-(aminomethyl)-7-(2-fluorophenyl)-3- azabicyclo[4.1.0]heptan-3-yl)-3-((5-chloroquinoxalin- 6-yl)thio)pyrazin-2-amine | 508.02 |
| 524 | 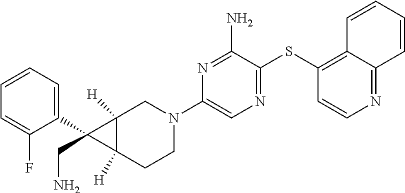 [SMILES: NC[C@]1(c2ccccc2F)[C@@H]2CCN(c3cnc(Sc4ccnc5ccccc45)c(N)n3)C[C@@H]21] | 6-((1S,6R,7R)-7-(aminomethyl)-7-(2-fluorophenyl)-3- azabicyclo[4.1.0]heptan-3-yl)-3-(quinolin-4- ylthio)pyrazin-2-amine | 472.58 |
| 525 | 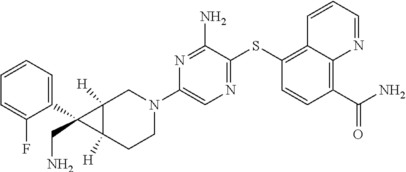 [SMILES: NC[C@]1(c2ccccc2F)[C@@H]2CCN(c3cnc(Sc4ccc(C(N)=O)c5ncccc45)c(N)n3)C[C@@H]21] | 5-((3-amino-5-((1S,6R,7R)-7-(aminomethyl)-7-(2- fluorophenyl)-3-azabicyclo[4.1.0]heptan-3-yl)pyrazin- 2-yl)thio)quinoline-8-carboxamide | 515.61 |
| 526 | 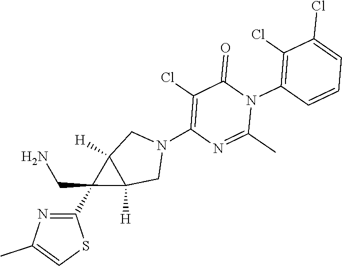 [SMILES: Cc1csc([C@@]2(CN)[C@@H]3CN(c4nc(C)n(-c5cccc(Cl)c5Cl)c(=O)c4Cl)C[C@@H]32)n1] | 6-((1R,5S,6r)-6-(aminomethyl)-6-(4-methylthiazol-2- yl)-3-azabicyclo[3.1.0]hexan-3-yl)-5-chloro-3-(2,3- dichlorophenyl)-2-methylpyrimidin-4(3H)-one | 496.84 |
| 527 | 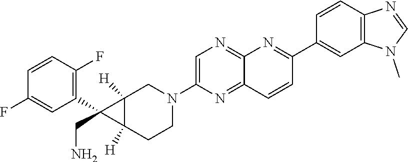 [SMILES: Cn1cnc2ccc(-c3ccc4nc(N5CC[C@@H]6[C@H](C5)[C@@]6(CN)c5cc(F)ccc5F)cnc4n3)cc21] | ((1S,6R,7R)-7-(2,5-difluorophenyl)-3-(6-(1-methyl- 1H-benzo[d]imidazol-6-yl)pyrido[2,3-b]pyrazin-2-yl)- 3-azabicyclo[4.1.0]heptan-7-yl)methanamine | 497.54 |
| 528 | 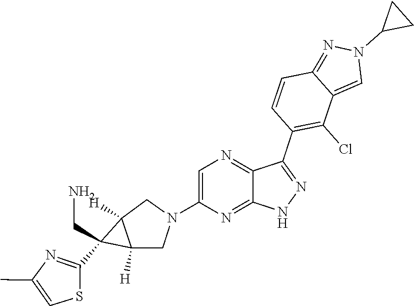 [SMILES: Cc1csc([C@@]2(CN)[C@@H]3CN(c4cnc5c(-c6ccc7nn(C8CC8)cc7c6Cl)n[nH]c5n4)C[C@@H]32)n1] | ((1R,5S,6r)-3-(3-(4-chloro-2-cyclopropyl-2H-indazol- 5-yl)-1H-pyrazolo[3,4-b]pyrazin-6-yl)-6-(4- methylthiazol-2-yl)-3-azabicyclo[3.1.0]hexan-6- yl)methanamine | 518.04 |
| 529 | 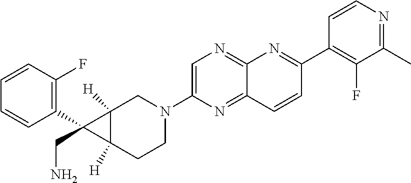 [SMILES: Cc1nccc(-c2ccc3nc(N4CC[C@@H]5[C@H](C4)[C@@]5(CN)c4ccccc4F)cnc3n2)c1F] | ((1S,6R,7R)-3-(6-(3-fluoro-2-methylpyridin-4- yl)pyrido[2,3-b]pyrazin-2-yl)-7-(2-fluorophenyl)-3- azabicyclo[4.1.0]heptan-7-yl)methanamine | 458.51 |
| 530 | 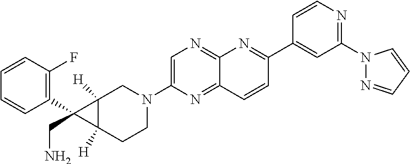 [SMILES: NC[C@]1(c2ccccc2F)[C@@H]2CCN(c3cnc4nc(-c5ccnc(-n6cccn6)c5)ccc4n3)C[C@@H]21] | ((1S,6R,7R)-3-(6-(2-(1H-pyrazol-1-yl)pyridin-4- yl)pyrido[2,3-b]pyrazin-2-yl)-7-(2-fluorophenyl)-3- azabicyclo[4.1.0]heptan-7-yl)methanamine | 492.55 |
| 531 | 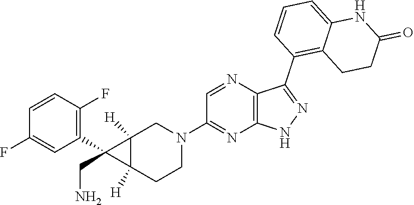 [SMILES: NC[C@]1(c2cc(F)ccc2F)[C@@H]2CCN(c3cnc4c(-c5cccc6c5CCC(=O)N6)n[nH]c4n3)C[C@@H]21] | 5-(6-((1S,6R,7R)-7-(aminomethyl)-7-(2,5- difluorophenyl)-3-azabicyclo[4.1.0]heptan-3-yl)-1H- pyrazolo[3,4-b]pyrazin-3-yl)-3,4-dihydroquinolin- 2(1H)-one | 501.53 |

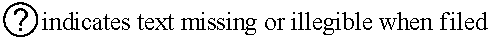

Exemplary and non-limiting processes for the synthesis of specific examples are reported herein below.

### Synthesis of Intermediates

### Intermediate 1: tert-butyl tetrahydro-[1,3,2]dioxathiolo[4,5-c]pyridine-5(4H)-carboxylate 2,2-dioxide

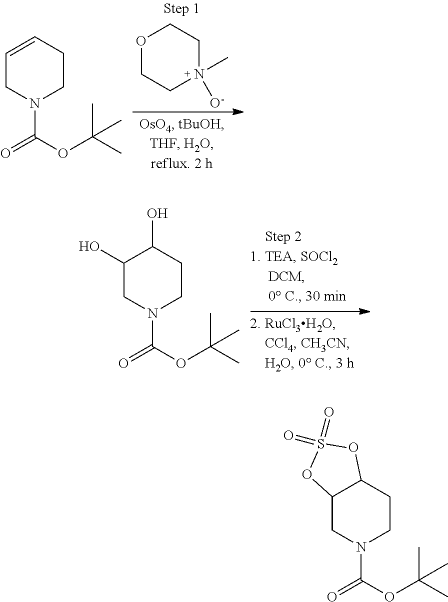

Step 1: tert-butyl 3,4-dihydroxypiperidine-1-carboxylate A solution of tert-butyl 3,6-dihydro-2H-pyridine-1-carboxylate (1.55 g, 8.46 mmol) and 4-methylmorpholine 4-oxide (1.49 g, 12.69 mmol) in a mixture of THF (8 mL), t-BuOH (5 mL) and H₂O (2 mL) at rt was treated with osmium(VIII) oxide (1.61 mL, 0.250 mmol; 4% in H₂O). The reaction mixture was heated at reflux for 2 h. After cooling the mixture was concentrated in vacuo and the residue dissolved in EtOAc. The organic phase was washed with brine, dried over Na₂SO₄, and concentrated under reduced pressure to give a residue which was purified by flash chromatography (0-100% EtOAc in petroleum ether) to afford the title compound as a colourless oil (1.68 g, 91%). LCMS (ES⁺) Method 2: m/z 240 (M+Na)⁺, RT 0.95 min.

### Step 2: tert-butyl tetrahydro-[1,3,2]dioxathiolo[4,5-c]pyridine-5(4H)-carboxylate 2,2-dioxide

A solution of tert-butyl 3,4-dihydroxypiperidine-1-carboxylate (1.68 g, 7.73 mmol) and TEA (4.31 mL, 30.93 mmol) in DCM (40 mL) at 0° C. was treated with SOCl₂ (0.84 mL, 11.6 mmol). The mixture was stirred at this temperature for 30 min then the reaction was quenched with H₂O (1.5 mL). Et₂O and additional H₂O were added, and the two phases were separated. The organic layer was washed with H₂O, NaHCO₃ sat. sol., brine, dried over Na₂SO₄, and concentrated in vacuo. The residue was dissolved in a mixture of CCl₄ (22 mL), MeCN (22 mL) and H₂O (33 mL) at 0° C. and the solution was treated with trichlororuthenium hydrate (34.8 mg, 0.150 mmol) and sodium periodate (3.31 g, 15.47 mmol). The reaction mixture was stirred at 0° C. for 3 h. Et₂O was added to the mixture and the phases were separated. The organic layer was then washed with H₂O, brine, dried over Na₂SO₄, and concentrated in vacuo to get a residue which was purified by flash chromatography (0-100% EtOAc in petroleum ether) to afford the title compound as a colourless oil (1.99 g, 92%). ¹H NMR (400 MHz, CDCl₃) δ 5.16-5.13 (m, 1H), 4.93 (br s, 1H), 3.95-3.90 (m, 1H), 3.88-3.82 (m, 1H), 3.62-3.57 (m, 1H), 3.46-3.42 (m, 1H), 2.33-2.27 (m, 1H), 2.09-2.05 (m, 1H), 1.48 (s, 9H). LCMS (ES⁺) Method 2: m/z 280 (M+H)⁺, RT 1.55 min.

### Intermediate 2: 7-((((benzyloxy)carbonyl)amino)methyl)-7-(pyridin-3-yl)-3-azabicyclo[4.1.0]heptan-3-ium chloride

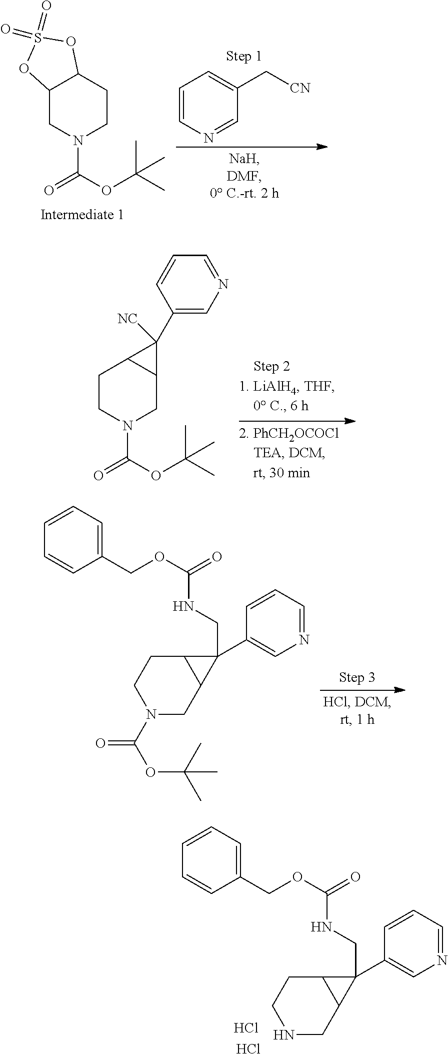

### Step 1: tert-butyl 7-cyano-7-(pyridin-3-yl)-3-azabicyclo[4.1.0]heptane-3-carboxylate

A suspension of NaH (60% in mineral oil; 38.9 mg, 0.970 mmol) in DMF (2 mL) was treated with 2-(3-pyridyl)acetonitrile (69.0 mg, 0.580 mmol) dissolved in DMF (2 mL) at 0° C. The orange suspension was stirred at 0° C. for 5 min and then treated with a solution of tert-butyl tetrahydro-[1,3,2]dioxathiolo[4,5-c]pyridine-5(4H)-carboxylate 2,2-dioxide (Intermediate 1; 136 mg, 0.490 mmol) in DMF (2 mL). The reaction mixture was warmed to rt and stirred for 2 h. Brine was added, and the mixture extracted with EtOAc (3×25 mL). Collected organics were washed with H₂O, brine, dried over Na₂SO₄, and concentrated in vacuo to get a residue which was purified by flash chromatography (0-100% EtOAc in petroleum ether) to afford the title compound as a yellow solid (50 mg, 34%). ¹H NMR (400 MHz, CDCl₃) δ 8.55-8.51 (m, 2H), 7.65 (d, J=8.0 Hz, 1H), 7.31-7.28 (m, 1H), 4.10-3.78 (m, 2H), 3.05-2.80 (m, 2H), 2.27 (br s, 1H), 2.10-2.02 (m, 2H), 1.95 (br s, 1H), 1.49 (s, 9H). LCMS (ES⁺) Method 2: m/z 300 (M+H)⁺, RT 1.08 min.

### Step 2: tert-butyl 7-((((benzyloxy)carbonyl)amino)methyl)-7-(pyridin-3-yl)-3-azabicyclo[4.1.0]heptane-3-carboxylate

A solution of tert-butyl 7-cyano-7-(pyridin-3-yl)-3-azabicyclo[4.1.0]heptane-3-carboxylate (25 mg, 0.084 mmol) in THF (2 mL) at 0° C. was treated with LiAlH₄ (1M in THF; 1.7 mL, 1.7 mmol) and the mixture was stirred at this temperature for 6 h. THF (5 mL) was added to the mixture followed by H₂O (80 uL) and NaOH (15%; 20 uL). The resulting slurry was stirred for 30 min at rt, filtered and the filtrate was washed with (DCM:EtOH=1:1; 10 mL). The mixture was concentrated under reduced pressure and the crude was dissolved in DCM (11 mL) and cooled at 0° C. TEA (0.12 mL, 0.850 mmol) and benzyl chloroformate (32 uL, 0.230 mmol) were added dropwise and mixture stirred for 30 min. Brine was added and the mixture was extracted with EtOAc (3×15 mL). The collected organics were washed with H₂O, brine, dried over Na₂SO₄, and concentrated in vacuo to obtain a residue which was purified by flash chromatography (0-100% EtOAc in petroleum ether) to afford the title compound as a yellow solid (10 mg, 27%). LCMS (ES⁺) Method 2: m/z 438 (M+H)⁺, RT 1.44 min.

### Step 3: 7-((((benzyloxy)carbonyl)amino)methyl)-7-(pyridin-3-yl)-3-azabicyclo[4.1.0]heptan-3-ium chloride

A solution of tert-butyl 7-((((benzyloxy)carbonyl)amino)methyl)-7-(pyridin-3-yl)-3-azabicyclo[4.1.0]heptane-3-carboxylate (10 mg, 0.020 mmol) in DCM (0.5 mL) was treated with HCl (371 uL, 1.49 mmol; 4M in 1,4-dioxane) and the solution was stirred at rt for 1 h. The mixture was concentrated under reduced pressure and the residue used as a crude. LCMS (ES⁺) Method 2: m/z 338 (M+H)⁺, RT 0.26 min.

### Intermediate 3: 7-((((benzyloxy)carbonyl)amino)methyl)-7-(2-fluorophenyl)-3-azabicyclo[4.1.0]heptan-3-ium chloride

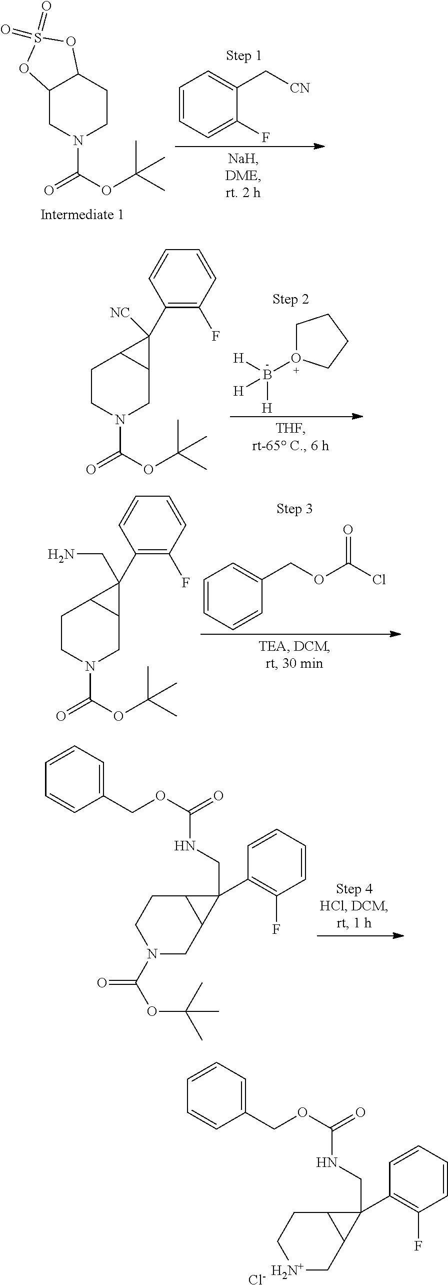

### Step 1: tert-butyl 7-cyano-7-(2-fluorophenyl)-3-azabicyclo[4.1.0]heptane-3-carboxylate

A suspension of NaH (60% in mineral oil; 156.4 mg, 3.91 mmol) in DME (4.5 mL) was treated with a solution of 15-crown-5 (33.1 mg, 0.150 mmol), 2-(2-fluorophenyl)acetonitrile (223.5 mg, 1.65 mmol) and tert-butyl tetrahydro-[1,3,2]dioxathiolo[4,5-c]pyridine-5(4H)-carboxylate 2,2-dioxide (Intermediate 1; 420 mg, 1.5 mmol) in DME (4.5 mL) dropwise in 30 min. The resulting orange mixture was stirred at rt for 2 h. H₂O (2 mL) was added, and the mixture was extracted with EtOAc (3×35 mL). The collected organics layers were washed with brine, dried over Na₂SO₄, and concentrated in vacuo to get a residue which was purified by flash chromatography (0-40% EtOAc in petroleum ether) to afford the title compound as a white solid (290 mg, 61%). ¹H NMR (400 MHz, CDCl₃) δ 7.35-7.26 (m, 2H), 7.15-7.06 (m, 2H), 4.20 (d, J=16.0 Hz, 1H), 3.87-3.72 (m, 2H), 3.01-2.92 (m, 1H), 2.24 (br s, 1H), 2.13-2.09 (m, 1H), 1.98-1.79 (m, 2H), 1.49 (s, 9H). LCMS (ES⁺) Method 2: m/z 339 (M+Na)⁺, RT 1.92 min.

### Step 2: tert-butyl 7-(aminomethyl)-7-(2-fluorophenyl)-3-azabicyclo[4.1.0]heptane-3-carboxylate

A solution of tert-butyl 7-cyano-7-(2-fluorophenyl)-3-azabicyclo[4.1.0]heptane-3-carboxylate (120 mg, 0.380 mmol) in THF (3 mL) was treated with borane tetrahydrofuran complex (2.84 mL, 2.84 mmol; 1M in THF) and the resulting mixture was stirred at 65° C. for 6 h. After cooling, EtOH (4 mL) was added, and mixture stirred at reflux for 4 h. The volatiles were removed by reduced pressure and the title compound was obtained as a crude and used without further purification. LCMS (ES⁺) Method 2: m/z 321 (M+H)⁺, RT 1.33 min.

### Step 3: tert-butyl 7-((((benzyloxy)carbonyl)amino)methyl)-7-(2-fluorophenyl)-3-azabicyclo[4.1.0]heptane-3-carboxylate

A solution of tert-butyl 7-(aminomethyl)-7-(2-fluorophenyl)-3-azabicyclo[4.1.0]heptane-3-carboxylate (139 mg, 0.430 mmol) in DCM (5 mL) was treated at 0° C. with TEA (0.18 mL, 1.3 mmol) and benzyl chloroformate (55.7 uL, 0.390 mmol) and mixture was warmed to rt and stirred for 30 min. Brine was added and the mixture was extracted with EtOAc (2×35 mL). The collected organics were washed with H₂O, brine, dried over Na₂SO₄, and concentrated in vacuo to get a residue which was purified by flash chromatography (0-100% EtOAc in petroleum ether) to afford the title compound as a colourless oil (70 mg, 35%). LCMS (ES⁺) Method 2: m/z 477 (M+Na)⁺, RT 2.28 min.

### Step 4: 7-((((benzyloxy)carbonyl)amino)methyl)-7-(2-fluorophenyl)-3-azabicyclo[4.1.0]heptan-3-ium chloride

A solution of tert-butyl 7-((((benzyloxy)carbonyl)amino)methyl)-7-(2-fluorophenyl)-3-azabicyclo[4.1.0]heptane-3-carboxylate (25 mg, 0.060 mmol) in DCM (1 mL) was treated with HCl (0.89 mL, 3.58 mmol; 4M in 1,4-dioxane) and the solution was stirred at rt for 1 h. Mixture was concentrated under reduced pressure and the residue used as a crude. LCMS (ES⁺) Method 2: m/z 355 (M+H)⁺, RT 1.28 min.

### Intermediate 4: 7-((((benzyloxy)carbonyl)amino)methyl)-7-(3-methylisoxazol-5-yl)-3-azabicyclo[4.1.0]heptan-3-ium chloride

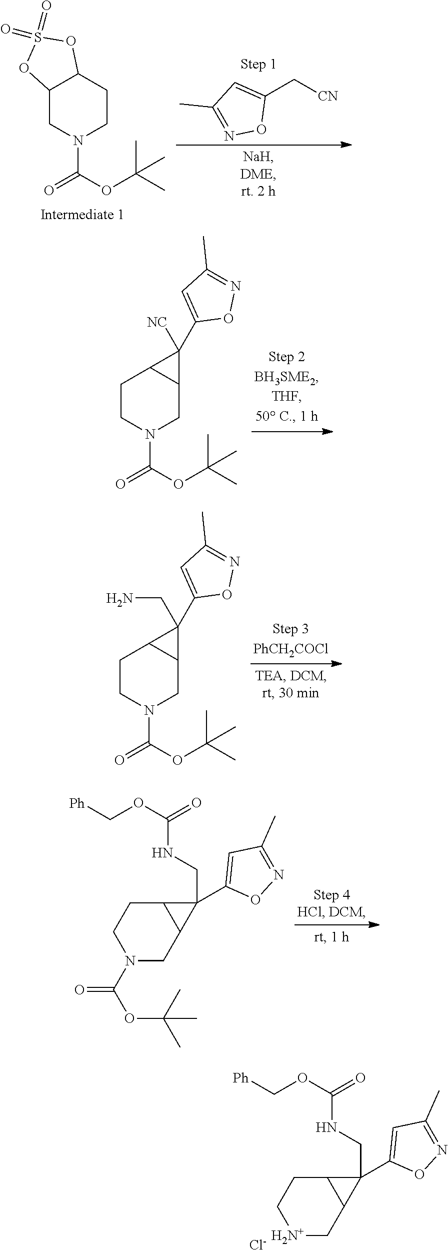

### Step 1: tert-butyl 7-cyano-7-(3-methylisoxazol-5-yl)-3-azabicyclo[4.1.0]heptane-3-carboxylate

A suspension of NaH (60% in mineral oil; 111.7 mg, 2.79 mmol) in DME (3.5 mL) was treated with a solution of tert-butyl tetrahydro-[1,3,2]dioxathiolo[4,5-c]pyridine-5(4H)-carboxylate 2,2-dioxide (Intermediate 1; 300 mg, 1.07 mmol), 2-(3-methyl-1,2-oxazol-5-yl)ethanenitrile (144.3 mg, 1.18 mmol) and 15-crown-5 (23.7 mg, 0.110 mmol) in DME (3.5 mL) dropwise in 30 min. The resulting orange mixture was stirred at rt for 2 h. H₂O (2 mL) was added, and the mixture was extracted with EtOAc (3×35 mL). The collected organics were washed with brine, dried over Na₂SO₄, and concentrated in vacuo to get a residue which was purified by flash chromatography (0-50% EtOAc in petroleum ether) to afford the title compound as a yellow oil (190 mg, 58%). ¹H NMR (400 MHz, CDCl₃) δ 6.21 (s, 1H), 4.26 (d, J=16.0 Hz, 1H), 4.09-3.82 (m, 1H), 3.64 (dd, J=4.0, 16.0 Hz, 1H), 2.91-2.75 (m, 1H), 2.38-2.35 (m, 1H), 2.29-2.15 (m, 2H), 2.28 (s, 3H), 2.05-1.98 (m, 1H), 1.48 (s, 9H). LCMS (ES⁺) Method 2: m/z 326 (M+Na)⁺, RT 1.67 min.

### Step 2: tert-butyl 7-(aminomethyl)-7-(3-methylisoxazol-5-yl)-3-azabicyclo[4.1.0]heptane-3-carboxylate

A solution of tert-butyl 7-cyano-7-(3-methylisoxazol-5-yl)-3-azabicyclo[4.1.0]heptane-3-carboxylate (120 mg, 0.400 mmol) in THF (7 mL) was treated with BMS (2 M in THF; 132 uL, 1.38 mmol) and the resulting mixture was stirred at 50° C. for 1 h. After cooling, EtOH (4 mL) was added and the mixture was stirred at reflux for 4 h. The volatiles were removed by reduced pressure and the title compound was obtained as a crude and used without further purification. LCMS (ES⁺) Method 2: m/z 308 (M+H)⁺, RT 1.11 min.

### Step 3: tert-butyl 7-((((benzyloxy)carbonyl)amino)methyl)-7-(3-methylisoxazol-5-yl)-3-azabicyclo[4.1.0]heptane-3-carboxylate

A solution of tert-butyl 7-(aminomethyl)-7-(3-methylisoxazol-5-yl)-3-azabicyclo[4.1.0]heptane-3-carboxylate (120 mg, 0.390 mmol) in DCM (4 mL), cooled at 0° C., was treated with TEA (0.16 mL, 1.17 mmol) and benzyl chloroformate (50 uL, 0.350 mmol) and the mixture was stirred at rt for 30 min. Brine was added and the mixture was extracted with EtOAc (2×35 mL). The collected organics were washed with H₂O, brine, dried over Na₂SO₄ and concentrated in vacuo to get a residue which was purified by flash chromatography (0-60% EtOAc in petroleum ether) to afford the title compound as a colourless oil (25 mg, 15%). LCMS (ES⁺) Method 2: m/z 464 (M+Na)⁺, RT 1.99 min.

### Step 4: 7-((((benzyloxy)carbonyl)amino)methyl)-7-(3-methylisoxazol-5-yl)-3-azabicyclo[4.1.0]heptan-3-ium chloride

A solution of tert-butyl 7-((((benzyloxy)carbonyl)amino)methyl)-7-(3-methylisoxazol-5-yl)-3-azabicyclo[4.1.0]heptane-3-carboxylate (25 mg, 0.060 mmol) in DCM (0.8 mL) was treated with HCl (4M in 1,4-dioxane; 0.92 mL, 3.68 mmol) and the solution was stirred at rt for 1 h. The mixture was concentrated under reduced pressure to afford the title compound as a white solid which was used as a crude. LCMS (ES⁺) Method 2: m/z 342 (M+H)⁺, RT 1.12 min.

### Intermediate 5: 7-((((benzyloxy)carbonyl)amino)methyl)-7-(3-fluorophenyl)-3-azabicyclo[4.1.0]heptan-3-ium chloride

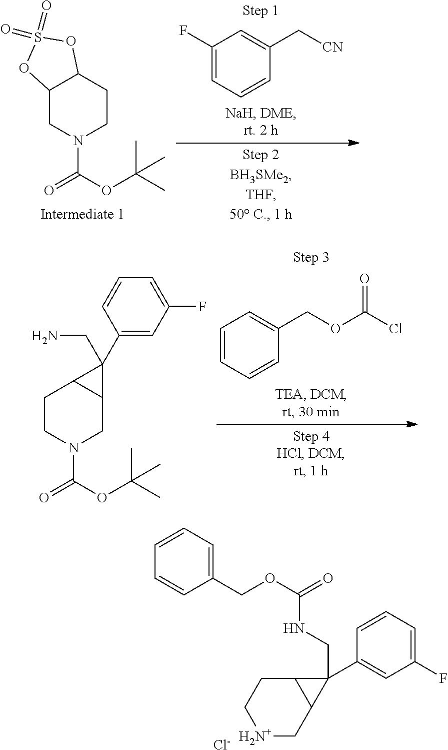

### Step 1: tert-butyl 7-cyano-7-(3-fluorophenyl)-3-azabicyclo[4.1.0]heptane-3-carboxylate

A solution of NaH (60% in mineral oil; 111.7 mg, 2.79 mmol) in DME (3 mL) was treated with a solution of 2-(3-fluorophenyl)acetonitrile (145.2 mg, 1.07 mmol), 15-crown-5 (23.7 mg, 0.110 mmol) and tert-butyl tetrahydro-[1,3,2]dioxathiolo[4,5-c]pyridine-5(4H)-carboxylate 2,2-dioxide (Intermediate 1; 300 mg, 1.07 mmol) in DME (2 mL) and the reaction mixture was stirred at rt for 2 h. H₂O was added and the mixture was extracted with EtOAc. The organic phase was washed with brine, dried over Na₂SO₄, and concentrated in vacuo to get a residue which was purified by flash chromatography (0-50% EtOAc in petroleum ether) to afford the title compound as a colorless oil (190 mg, 56%). ¹H NMR (400 MHz, CDCl₃) δ 7.36-7.30 (m, 1H), 7.10 (d, J=8.0 Hz, 1H), 7.01-6.96 (m, 2H), 4.17 (d, J=12.0 Hz, 1H), 4.01-3.74 (m, 2H), 3.00-2.85 (m, 1H), 2.29-2.22 (m, 1H), 2.10-1.89 (m, 2H), 1.66 (br s, 1H), 1.51 (s, 9H). LCMS (ES⁺) Method 2: m/z 339 (M+Na)⁺, RT 2.0 min.

### Step 2: tert-butyl 7-(aminomethyl)-7-(3-fluorophenyl)-3-azabicyclo[4.1.0]heptane-3-carboxylate

A solution of tert-butyl 7-cyano-7-(3-fluorophenyl)-3-azabicyclo[4.1.0]heptane-3-carboxylate (190 mg, 0.600 mmol) in THF (12 mL) was treated with BMS (2M in THF; 285 uL, 3 mmol) and the resulting mixture was stirred at 65° C. for 30 min. After cooling EtOH (4 mL) was added and the mixture stirred at reflux for 4 h. After cooling, the volatiles were removed by reduced pressure and the title compound was obtained as a colorless oil (192 mg, 99%) and used without further purification. LCMS (ES⁺) Method 2: m/z 321 (M+H)⁺, RT 1.15 min.

### Step 3: tert-butyl 7-((((benzyloxy)carbonyl)amino)methyl)-7-(3-fluorophenyl)-3-azabicyclo[4.1.0]heptane-3-carboxylate

A solution of tert-butyl 7-(aminomethyl)-7-(3-fluorophenyl)-3-azabicyclo[4.1.0]heptane-3-carboxylate (192 mg, 0.600 mmol) in DCM (5 mL) cooled at 0° C. was treated with TEA (0.25 mL, 1.8 mmol) and benzyl chloroformate (68 uL, 0.480 mmol) and the mixture was stirred at rt for 45 min. Brine was added and the mixture was extracted with EtOAc (2×35 mL). The collected organics were washed with H₂O, brine, dried over Na₂SO₄, and concentrated in vacuo to get a residue which was purified by flash chromatography (0-100% EtOAc in petroleum ether) to afford the title compound as a colorless oil (112 mg, 41%). LCMS (ES⁺) Method 2: m/z 477 (M+Na)⁺, RT 2.30 min.

### Step 4: 7-((((benzyloxy)carbonyl)amino)methyl)-7-(3-fluorophenyl)-3-azabicyclo[4.1.0]heptan-3-ium chloride

A solution of tert-butyl 7-((((benzyloxy)carbonyl)amino)methyl)-7-(3-fluorophenyl)-3-azabicyclo[4.1.0]heptane-3-carboxylate (112 mg, 0.250 mmol) in DCM (4.0 mL) was treated with HCl (4M in 1,4-dioxane; 4.0 mL) and the solution stirred at rt for 2 h. The mixture was concentrated under reduced pressure to afford the title compound as a white solid which was used as a crude. LCMS (ES⁺) Method 2: m/z 355 (M+H)⁺, RT 1.14 min.

### Intermediate 6: 7-((((benzyloxy)carbonyl)amino)methyl)-7-(5-methylisoxazol-3-yl)-3-azabicyclo[4.1.0]heptan-3-ium chloride

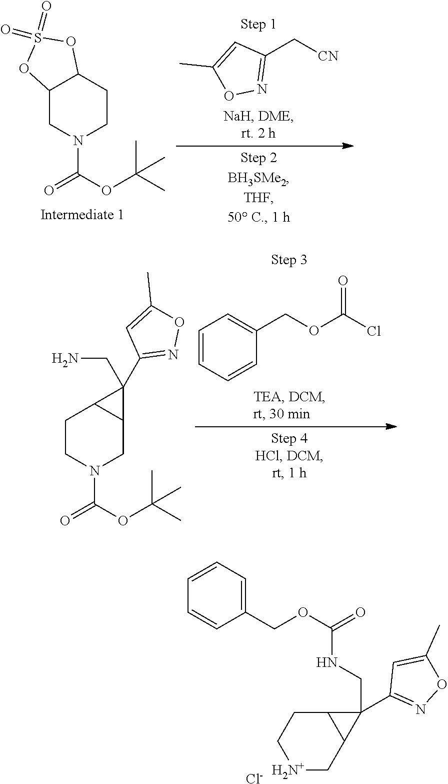

### Step 1: tert-butyl 7-cyano-7-(5-methylisoxazol-3-yl)-3-azabicyclo[4.1.0]heptane-3-carboxylate

A solution of NaH (60% in mineral oil; 111.7 mg, 2.79 mmol) in DME (3.5 mL) was treated with a solution of tert-butyl tetrahydro-[1,3,2]dioxathiolo[4,5-c]pyridine-5(4H)-carboxylate 2,2-dioxide (Intermediate 1; 300 mg, 1.07 mmol), 2-(5-methyl-1,2-oxazol-3-yl)ethanenitrile (144.3 mg, 1.18 mmol) and 15-crown-5 (23.7 mg, 0.110 mmol) in DME (3.5 mL) and the reaction mixture was stirred at rt for 2 h. H₂O (2 mL) was added and the mixture was extracted with EtOAc (3×35 mL). The collected organics were washed with brine, dried over Na₂SO₄, and concentrated in vacuo to get a residue which was purified by flash chromatography (0-50% EtOAc in petroleum ether) to afford the title compound as a yellow oil (177 mg, 54%). ¹H NMR (400 MHz, CDCl₃) δ 6.18 (s, 1H), 4.23 (d, J=16.0 Hz, 1H), 3.98-3.80 (m, 1H), 3.66 (dd, J=4.0, 16.0 Hz, 1H), 2.93-2.79 (m, 1H), 2.41 (s, 3H), 2.30-2.12 (m, 3H), 2.03-1.98 (m, 1H), 1.48 (s, 9H). LCMS (ES⁺) Method 2: m/z 326 (M+Na)⁺, RT 1.71 min.

### Step 2: tert-butyl 7-(aminomethyl)-7-(5-methylisoxazol-3-yl)-3-azabicyclo[4.1.0]heptane-3-carboxylate

A solution of tert-butyl 7-cyano-7-(5-methylisoxazol-3-yl)-3-azabicyclo[4.1.0]heptane-3-carboxylate (177 mg, 0.580 mmol) in THF (7 mL) was treated with BMS (2M in THF; 194 uL, 2.04 mmol) and the resulting mixture was stirred at 50° C. for 1 h. After cooling, EtOH (4 mL) was added and mixture stirred at reflux for 4 h. The volatiles were removed by reduced pressure and the title compound was used without further purification. LCMS (ES⁺) Method 2: m/z 308 (M+H)⁺, RT 1.15 min.

### Step 3: tert-butyl 7-((((benzyloxy)carbonyl)amino)methyl)-7-(5-methylisoxazol-3-yl)-3-azabicyclo[4.1.0]heptane-3-carboxylate

A solution of tert-butyl 7-(aminomethyl)-7-(5-methylisoxazol-3-yl)-3-azabicyclo[4.1.0]heptane-3-carboxylate (188 mg, 0.610 mmol) in DCM (6 mL) cooled at 0° C. was treated with TEA (0.26 mL, 1.83 mmol) and benzyl chloroformate (79 uL, 0.550 mmol) and mixture was stirred at rt for 30 min. Brine was added and mixture extracted with EtOAc (2×35 mL). The collected organics were washed with H₂O, brine, dried over Na₂SO₄, and concentrated in vacuo to get a residue which was purified by flash chromatography (0-60% EtOAc in petroleum ether) to afford the title compound as a colorless oil (57 mg, 21%). LCMS (ES⁺) Method 2: m/z 464 (M+Na)⁺, RT 2.05 min.

### Step 4: 7-((((benzyloxy)carbonyl)amino)methyl)-7-(5-methylisoxazol-3-yl)-3-azabicyclo[4.1.0]heptan-3-ium chloride

A solution of tert-butyl 7-((((benzyloxy)carbonyl)amino)methyl)-7-(5-methylisoxazol-3-yl)-3-azabicyclo[4.1.0]heptane-3-carboxylate (31 mg, 0.070 mmol) in DCM (1 mL) was treated with HCl (4M in 1,4-dioxane; 1.14 mL, 4.56 mmol) and the solution was stirred at rt for 1 h. The mixture was concentrated under reduced pressure to afford the title compound as a white solid which was used as a crude. LCMS (ES⁺) Method 2: m/z 342 (M+H)⁺, RT 1.20 min.

### Intermediate 7: 7-((((benzyloxy)carbonyl)amino)methyl)-7-(4-fluorophenyl)-3-azabicyclo[4.1.0]heptan-3-ium chloride

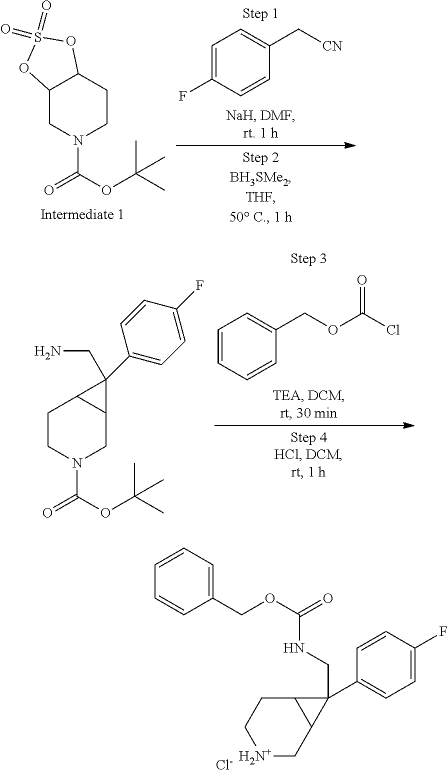

### Step 1: tert-butyl 7-cyano-7-(4-fluorophenyl)-3-azabicyclo[4.1.0]heptane-3-carboxylate

A suspension of NaH (60% in mineral oil; 93 mg, 2.33 mmol) in DMF (2.5 mL) cooled at 0° C. was treated with 2-(4-fluorophenyl)acetonitrile (118 uL, 0.980 mmol) in DMF (2.5 mL). A solution of tert-butyl tetrahydro-[1,3,2]dioxathiolo[4,5-c]pyridine-5(4H)-carboxylate 2,2-dioxide (Intermediate 1; 250 mg, 0.900 mmol) in DMF (2.5 mL) was then added to the suspension and the resulting orange mixture was stirred at rt for 1 h. H₂O (4 mL) was added and the mixture was extracted with EtOAc (3×35 mL). The collected organics were washed with brine, dried over Na₂SO₄, and concentrated in vacuo to get a residue which was purified by flash chromatography (0-40% EtOAc in petroleum ether) to afford the title compound as a yellow oil (135 mg, 48%). ¹H NMR (400 MHz, CDCl₃) δ 7.27-7.25 (m, 2H), 7.06-7.02 (m, 2H), 4.17-4.12 (m, 1H), 3.95-3.72 (m, 2H), 3.00-2.86 (m, 1H), 2.24 (br s, 1H), 2.07-2.04 (m, 1H), 1.95-1.83 (m, 2H), 1.49 (s, 9H). LCMS (ES⁺) Method 2: m/z 339 (M+Na)⁺, RT 2.05 min.

### Step 2: tert-butyl 7-(aminomethyl)-7-(4-fluorophenyl)-3-azabicyclo[4.1.0]heptane-3-carboxylate

A solution of tert-butyl 7-cyano-7-(4-fluorophenyl)-3-azabicyclo[4.1.0]heptane-3-carboxylate (135 mg, 0.430 mmol) in THF (5.5 mL) was treated with BMS (2M in THF; 142 uL, 1.49 mmol) and the resulting mixture was stirred at 50° C. for 1 h. After cooling, EtOH (4 mL) was added and the mixture was stirred at reflux for 4 h. The volatiles were removed by reduced pressure and the title compound was used without further purification. LCMS (ES⁺) Method 2: m/z 321 (M+H)⁺, RT 1.35 min.

### Step 3: tert-butyl 7-((((benzyloxy)carbonyl)amino)methyl)-7-(4-fluorophenyl)-3-azabicyclo[4.1.0]heptane-3-carboxylate

A solution of tert-butyl 7-(aminomethyl)-7-(4-fluorophenyl)-3-azabicyclo[4.1.0]heptane-3-carboxylate (145 mg, 0.450 mmol) in DCM (6 mL) cooled at 0° C. was treated with TEA (189 uL, 1.36 mmol) and benzyl chloroformate (58 uL, 0.410 mmol) were added and the mixture was stirred at rt for 30 min. Brine was added and the mixture was extracted with EtOAc (2×35 mL). The collected organics were washed with H₂O, brine, dried over Na₂SO₄ and concentrated in vacuo to get a residue which was purified by flash chromatography (0-60% EtOAc in petroleum ether) to afford the title compound as a colorless oil (38 mg, 18%). LCMS (ES⁺) Method 2: m/z 477 (M+Na)⁺, RT 2.27 min.

### Step 4: 7-((((benzyloxy)carbonyl)amino)methyl)-7-(4-fluorophenyl)-3-azabicyclo[4.1.0]heptan-3-ium chloride

A solution of tert-butyl 7-((((benzyloxy)carbonyl)amino)methyl)-7-(4-fluorophenyl)-3-azabicyclo[4.1.0]heptane-3-carboxylate (38 mg, 0.090 mmol) in DCM (0.8 mL) was treated with HCl (4M in 1,4-dioxane; 1.39 mL, 5.58 mmol) and the solution was stirred at rt for 1 h. The mixture was concentrated under reduced pressure to afford the title compound as a white solid which was used as a crude. LCMS (ES⁺) Method 2: m/z 355 (M+H)⁺, RT 1.37 min.

### Intermediate 8: benzyl tetrahydro-[1,3,2]dioxathiolo[4,5-c]pyridine-5(4H)-carboxylate 2,2-dioxide

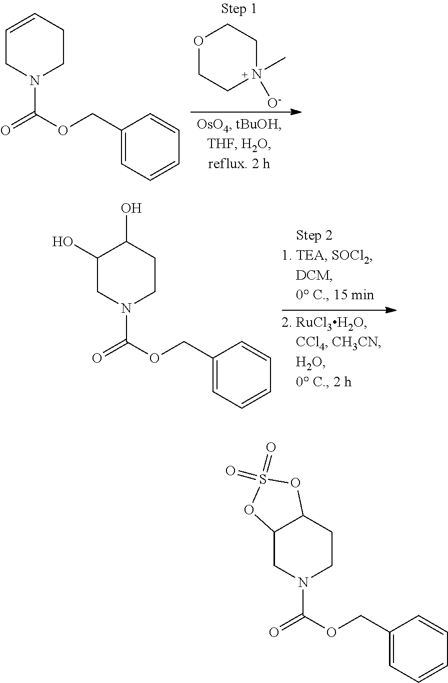

### Step 1: benzyl 3,4-dihydroxypiperidine-1-carboxylate

A solution of benzyl 3,6-dihydropyridine-1(2H)-carboxylate (2 g, 9.21 mmol) and 4-methylmorpholine 4-oxide (1.62 g, 13.81 mmol) in a mixture of THF (10 mL), tert-BuOH (6 mL) and H₂O (2.5 mL) at rt was treated with osmium(VIII) oxide (4% in H₂O; 1.76 mL, 0.280 mmol). The reaction mixture was heated at reflux for 2 h and then concentrated in vacuo and the residue was taken up in EtOAc. The organic phase was washed with brine, dried over Na₂SO₄, and concentrated under reduced pressure to give a residue which was purified by flash chromatography (0-100% EtOAc in petroleum ether) to afford the title compound as a colorless oil (2.31 g, 100%). ¹H-NMR (400 MHz, CDCl₃) δ 7.39-7.33 (m, 5H), 5.14 (s, 2H), 3.90-3.87 (m, 1H), 3.79 (br s, 1H), 3.69-3.63 (m, 2H), 3.52-3.49 (m, 1H), 3.37-3.33 (m, 1H), 2.21 (br s, 2H), 1.89-1.82 (m, 1H), 1.71 (br s, 1H). LCMS (ES⁺) Method 2: m/z 274 (M+Na)⁺, RT 1.08 min.

### Step 2: benzyl tetrahydro-[1,3,2]dioxathiolo[4,5-c]pyridine-5(4H)-carboxylate 2,2-dioxide

A solution of benzyl 3,4-dihydroxypiperidine-1-carboxylate (2.31 g, 9.35 mmol) and TEA (5.21 mL, 37.41 mmol) in DCM (48 mL) at 0° C. was treated with SOCl₂ (1.02 mL, 14.03 mmol). The mixture was stirred at this temperature for 15 min then the reaction was quenched with H₂O (5 mL). Et₂O and additional H₂O were added, and the phases were separated. The organic layer was washed with H₂O, NaHCO₃ sat. sol., brine, dried over Na₂SO₄, and concentrated in vacuo. The residue was dissolved in a mixture of CCl₄ (27 mL), MeCN (27 mL) and H₂O (45 mL) at 0° C. and the solution was treated with trichlororuthenium hydrate (42.1 mg, 0.190 mmol) and sodium periodate (4 g, 18.7 mmol). The reaction mixture was stirred at 0° C. for 2 h. Et₂O was added to the mixture and the two phases were separated. The organic layer was then washed with H₂O, brine, dried over Na₂SO₄ and concentrated in vacuo to get a residue which was purified by flash chromatography (0-60% EtOAc in petroleum ether) to afford the title compound as a pale-yellow oil (2.7 g, 92%). ¹H NMR (400 MHz, CDCl₃) δ 7.41-7.35 (m, 5H), 5.20-5.13 (m, 1H), 5.16 (s, 2H), 4.94 (br s, 1H), 4.08-3.99 (m, 1H), 3.93-3.89 (m, 1H), 3.72-3.66 (m, 1H), 3.53-3.48 (m, 1H), 2.33-2.30 (m, 1H), 2.08 (br s, 1H). LCMS (ES⁺) Method 2: m/z 314 (M+H)⁺, RT 1.60 min.

### Intermediate 9: tert-butyl ((7-(1-methyl-1H-1,2,3-triazol-4-yl)-3-azabicyclo[4.1.0]heptan-7-yl)methyl)carbamate

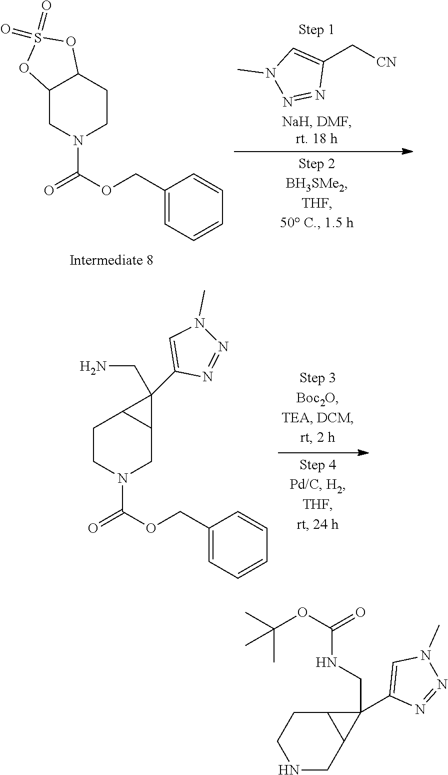

### Step 1: benzyl 7-cyano-7-(1-methyl-1H-1, 2, 3-triazol-4-yl)-3-azabicyclo[4.1.0]heptane-3-carboxylate

A suspension of NaH (60% in mineral oil; 83 mg, 2.07 mmol) in DMF (2.0 mL) cooled at 0° C. was treated with 2-(1-methyl-1H-1,2,3-triazol-4-yl)acetonitrile (107.2 mg, 0.880 mmol) in DMF (2.0 mL). A solution of benzyl tetrahydro-[1,3,2]dioxathiolo[4,5-c]pyridine-5(4H)-carboxylate 2,2-dioxide (Intermediate 8; 250 mg, 0.800 mmol) in DMF (2.0 mL) was then added to the suspension and the resulting orange mixture was stirred at rt for 18 h. H₂O (4 mL) was added and the mixture extracted with EtOAc (3×35 mL). The collected organics were washed with brine, dried over Na₂SO₄, and concentrated in vacuo to get a residue which was purified by flash chromatography (0-100% EtOAc in petroleum ether) to afford the title compound as a yellow oil (75 mg, 28%). ¹H NMR (400 MHz, CDCl₃) δ 7.65 (s, 1H), 7.39-7.33 (m, 5H), 5.21-5.10 (m, 2H), 4.32 (d, J=16.0 Hz, 1H), 4.08 (s, 3H), 4.08-4.00 (m, 1H), 3.75 (dd, J=4.0, 16.0 Hz, 1H), 2.38-2.26 (m, 3H), 2.10-2.04 (m, 1H), 1.71 (br s, 1H). LCMS (ES⁺) Method 2: m/z 360 (M+Na)⁺, RT 1.43 min.

### Step 2: benzyl 7-(aminomethyl)-7-(I-methyl-1H-1,2,3-triazol-4-yl)-3-azabicyclo[4.1.0]heptane-3-carboxylate

A solution of benzyl 7-cyano-7-(1-methyl-1H-1,2,3-triazol-4-yl)-3-azabicyclo[4.1.0]heptane-3-carboxylate (75 mg, 0.220 mmol) in THF (2.5 mL) was treated with BMS (2M in THF; 389 uL, 0.780 mmol) and the resulting mixture was stirred at 50° C. for 1.5 h. After cooling, EtOH (4 mL) was added and the mixture stirred at reflux for 2 h. After cooling, the solvent was then removed by reduced pressure and the title compound was used without further purification. LCMS (ES⁺) Method 2: m/z 342 (M+H)⁺, RT 1.07 min.

### Step 3: benzyl 7-(((tert-butoxycarbonyl)amino)methyl)-7-(1-methyl-1H-1,2,3-triazol-4-yl)-3-azabicyclo[4.1.0]heptane-3-carboxylate

A solution of benzyl 7-(aminomethyl)-7-(1-methyl-1H-1,2,3-triazol-4-yl)-3-azabicyclo[4.1.0]heptane-3-carboxylate (89 mg, 0.260 mmol) and TEA (50.9 uL, 0.360 mmol) in DCM (2.5 mL) was treated with (Boc)₂O (62.6 mg, 0.290 mmol) and the mixture was stirred at rt for 2 h. H₂O (3 mL) was added and the mixture extracted with EtOAc (2×35 mL). The collected organics were washed with H₂O, brine, dried over Na₂SO₄, and concentrated in vacuo to get a residue which was purified by flash chromatography (0-100% EtOAc in petroleum ether) to afford the title compound as a colorless oil (53 mg, 46%). LCMS (ES⁺) Method 2: m/z 464 (M+Na)⁺, RT 1.72 min.

### Step 4: tert-butyl ((7-(1-methyl-1H-1,2,3-triazol-4-yl)-3-azabicyclo[4.1.0]heptan-7-yl)methyl)carbamate

A solution of benzyl 7-(((tert-butoxycarbonyl)amino)methyl)-7-(1-methyl-1H-1,2,3-triazol-4-yl)-3-azabicyclo[4.1.0]heptane-3-carboxylate (53 mg, 0.120 mmol) in THF (3 mL) was treated with Pd/C (20 mg, 0.120 mmol) and the resulting suspension was stirred under H₂ atmosphere at rt for 24 h. After N₂ fluxing to remove H₂, the catalyst was filtered on a pad of solka-floc which was washed with MeOH. After volatiles removal under reduced pressure, the title compound was obtained as a colourless oil (26 mg, 70%) and used as crude. LCMS (ES⁺) Method 2: m/z 308 (M+H)⁺, RT 0.70 min.

### Intermediate 10: tert-butyl ((7-(4-methylisoxazol-3-yl)-3-azabicyclo[4.1.0]heptan-7-yl)methyl)carbamate

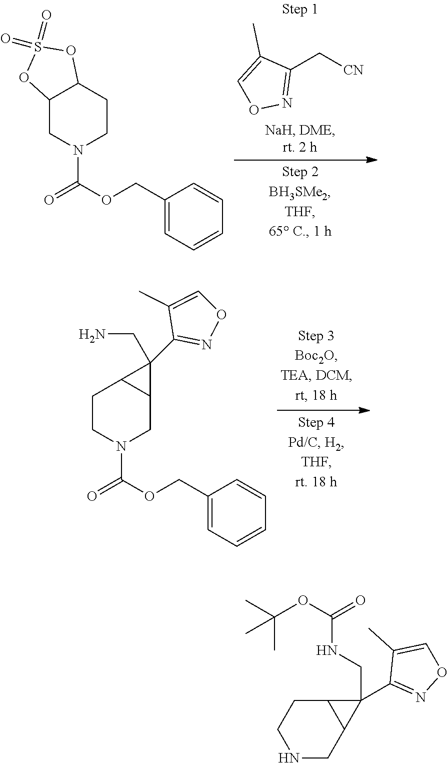

### Step 1: benzyl 7-cyano-7-(4-methylisoxazol-3-yl)-3-azabicyclo[4.1.0]heptane-3-carboxylate

A suspension of NaH (60% in mineral oil; 99.6 mg, 2.49 mmol) in DME (3.2 mL) cooled at 0° C. was treated with 2-(4-methyl-1,2-oxazol-3-yl)ethanenitrile (128.6 mg, 1.05 mmol) followed by benzyl tetrahydro-[1,3,2]dioxathiolo[4,5-c]pyridine-5(4H)-carboxylate 2,2-dioxide (Intermediate 8; 300 mg, 0.960 mmol) and 15-crown-5(0.02 mL, 0.100 mmol) in DME (3.2 mL) and the resulting orange mixture was stirred at rt for 2 h. H₂O (2 mL) was added and the mixture extracted with EtOAc (3×35 mL). The collected organics were washed with brine, dried over Na₂SO₄, and concentrated in vacuo to get a residue which was purified by flash chromatography (0-100% EtOAc in petroleum ether) to afford the title compound as a yellow oil (120 mg, 37%). LCMS (ES⁺) Method 2: m/z 338 (M+H)⁺, RT 1.76 min.

### Step 2: benzyl 7-(aminomethyl)-7-(4-methylisoxazol-3-yl)-3-azabicyclo[4.1.0]heptane-3-carboxylate

A solution of benzyl 7-cyano-7-(4-methylisoxazol-3-yl)-3-azabicyclo[4.1.0]heptane-3-carboxylate (120 mg, 0.360 mmol) in THF (9.0 mL) was treated with BMS (2M in THF; 0.89 mL, 1.78 mmol) and the resulting mixture was stirred at 65° C. for 1 h. After cooling, EtOH was added, and mixture stirred at reflux for 4 h. The volatiles were removed under reduced pressure and the title compound was obtained as a colorless oil and used without further purification. LCMS (ES⁺) Method 2: m/z 342 (M+H)⁺, RT 1.07 min.

### Step 3: benzyl 7-(((tert-butoxycarbonyl)amino)methyl)-7-(4-methylisoxazol-3-yl)-3-azabicyclo[4.1.0]heptane-3-carboxylate

A solution of benzyl 7-(aminomethyl)-7-(4-methylisoxazol-3-yl)-3-azabicyclo[4.1.0]heptane-3-carboxylate (121 mg, 0.350 mmol) and TEA (0.1 mL, 0.710 mmol) in DCM (1.8 mL) was treated with (Boc)₂O (0.1 mL, 0.430 mmol) and the mixture was stirred at rt for 18 h. NaHCO₃ sat. sol. was added and mixture was extracted with EtOAc. The organic phase was washed with brine, dried over Na₂SO₄, and concentrated in vacuo to get a residue which was purified by flash chromatography (0-100% EtOAc in petroleum ether) to afford the title compound as a colorless oil (80 mg, 51%). LCMS (ES⁺) Method 2: m/z 442 (M+H)⁺, RT 2.0 min.

### Step 4: tert-butyl ((7-(4-methylisoxazol-3-yl)-3-azabicyclo[4.1.0]heptan-7-yl)methyl)carbamate (Intermediate 10)

A solution of benzyl 7-(((tert-butoxycarbonyl)amino)methyl)-7-(4-methylisoxazol-3-yl)-3-azabicyclo[4.1.0]heptane-3-carboxylate (80 mg, 0.180 mmol) in THF (1.5 mL) was treated with Pd/C (30 mg) and the resulting suspension was stirred under H₂ atmosphere at rt for 18 h. After N₂ fluxing to remove H₂, the catalyst was filtered on a pad of solka-floc which was washed with MeOH. After volatiles removal under reduced pressure, the title compound was obtained as a colorless oil and used as crude. LCMS (ES⁺) Method 2: m/z 308 (M+H)⁺, RT 0.91 min.

### Intermediate 11: tert-butyl (3-chloro-4-(4,4,5,5-tetramethyl-1,3,2-dioxaborolan-2-yl)pyridin-2-yl)(methyl)carbamate

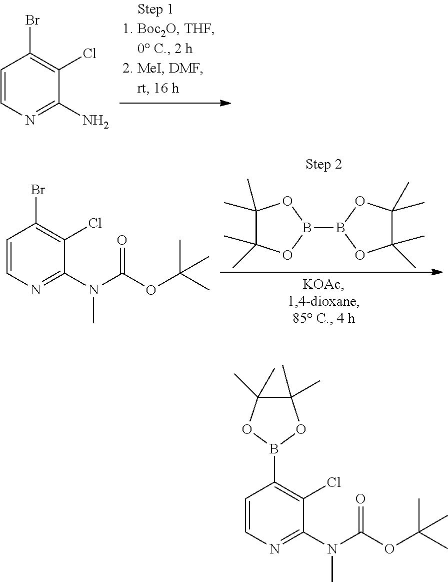

### Step 1: tert-butyl (4-bromo-3-chloropyridin-2-yl)(methyl)carbamate

A solution of 4-bromo-3-chloropyridin-2-amine (200 mg, 0.960 mmol) and (Boc)₂O (274 mg, 1.253 mmol) in THF (4 mL) at 0° C. was treated dropwise with [bis(trimethylsilyl)amino]sodium (2.41 mL, 2.41 mmol).

The resulting mixture was stirred at rt for 2 h. DMF (5 mL) and Mel (90.0 uL, 1.45 mmol) were added and the mixture stirred at rt for 16 h. H₂O (5 mL) was added dropwise and the mixture was extracted with Et₂O (2×50 mL). The collected organics were washed with brine, dried over Na₂SO₄, and concentrated in vacuo to get a residue which was purified by flash chromatography (0-20% EtOAc in petroleum ether) to afford the title compound as a pale-yellow oil (295 mg, 95%). ¹H NMR (400 MHz, DMSO-d₆) δ 8.30 (d, J=4.1 Hz, 1H), 7.82 (d, J=4.0 Hz, 1H), 3.13 (s, 3H), 1.35 (s, 9H). LCMS (ES⁺) Method 2: m/z 321 (M+H)⁺, RT 2.02 min.

### Step 2: tert-butyl (3-chloro-4-(4,4,5,5-tetramethyl-1,3,2-dioxaborolan-2-yl)pyridin-2-yl)(methyl)carbamate

A suspension of tert-butyl (4-bromo-3-chloropyridin-2-yl)(methyl)carbamate (79 mg, 0.800 mmol), Pd(dppf)Cl₂·DCM (33.3 mg, 0.040 mmol), KOAc (79 mg, 0.805 mmol) and B₂pin₂ (112.4 mg, 0.440 mmol) in 1,4-dioxane (4 mL) was stirred at 85° C. for 4 h. After cooling, the mixture was filtered on a pad of solka-floc and the filtrate concentrated in vacuo to get a residue which was purified by flash chromatography (0-50% EtOAc in petroleum ether) to afford the title compound as a white powder (99 mg, 67%). ¹H NMR (400 MHz, DMSO-d₆) δ 8.42 (d, J=4.0 Hz, 1H), 7.48 (d, J=4.0 Hz, 1H), 3.10 (s, 3H), 1.33 (s, 9H), 1.16 (s, 6H), 1.07 (s, 6H). LCMS (ES⁺) Method 2: m/z 285 (M−H of the boronic acid), RT 1.20 min.

### Intermediate 12: tert-butyl ((7-(5-methylisoxazol-3-yl)-3-azabicyclo[4.1.0]heptan-7-yl)methyl)carbamate

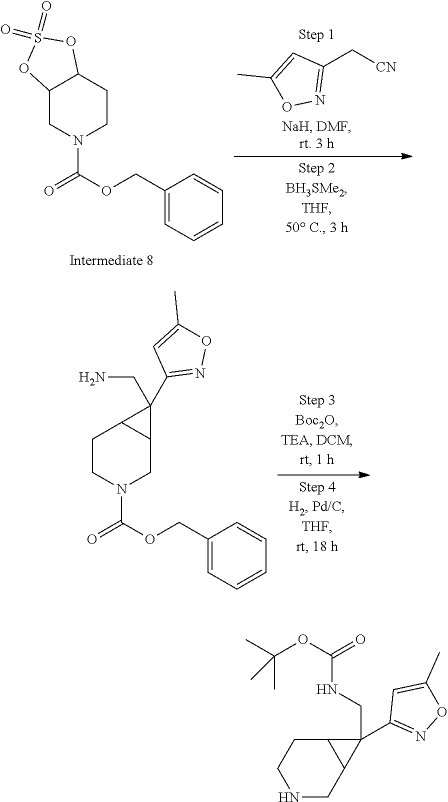

### Step 1: benzyl 7-cyano-7-(5-methylisoxazol-3-yl)-3-azabicyclo[4.1.0]heptane-3-carboxylate

A solution of NaH (60% in mineral oil; 261 mg, 6.52 mmol) in DME (7.5 mL) was treated with a solution of benzyl tetrahydro-[1,3,2]dioxathiolo[4,5-c]pyridine-5(4H)-carboxylate 2,2-dioxide (Intermediate 8; 785 mg, 2.51 mmol), 2-(5-methyl-1,2-oxazol-3-yl)ethanenitrile (337 mg, 2.76 mmol) and 15-crown-5 (50 uL, 0.250 mmol) in DME (7.5 mL) and the reaction mixture was stirred at rt for 3 h. H₂O (4 mL) was added and mixture extracted with EtOAc (2×30 mL). The collected organics were washed with brine, dried over Na₂SO₄, and concentrated in vacuo to get a residue which was purified by flash chromatography (0-50% EtOAc in petroleum ether) to afford the title compound as a yellow oil (410 mg, 48%). ¹H NMR (400 MHz, CDCl₃) δ 7.38-7.35 (m, 5H), 6.19 (s, 1H), 5.23-5.10 (m, 2H), 4.31 (d, J=16.0 Hz, 1H), 4.05-3.70 (m, 2H), 3.02-2.87 (m, 1H), 2.42 (s, 3H), 2.31-2.15 (m, 3H), 2.11-2.07 (m, 1H). LCMS (ES⁺) Method 2: m/z 360 (M+Na)⁺, RT 1.74 min.

### Step 2: benzyl 7-(aminomethyl)-7-(5-methylisoxazol-3-yl)-3-azabicyclo[4.1.0]heptane-3-carboxylate

A solution of benzyl 7-cyano-7-(5-methylisoxazol-3-yl)-3-azabicyclo[4.1.0]heptane-3-carboxylate (405 mg, 1.2 mmol) in THF (12 mL) was treated with BMS (2M in THF; 2.1 mL, 4.2 mmol) and the resulting mixture was stirred at 50° C. for 3 h. After cooling, EtOH (5 mL) was added, and the mixture stirred at reflux for 15 h. The volatiles were removed under reduced pressure and the title compound was used without further purification. LCMS (ES⁺) Method 2: m/z 342 (M+H)⁺, RT 1.22 min.

### Step 3: benzyl 7-(((tert-butoxycarbonyl)amino)methyl)-7-(5-methylisoxazol-3-yl)-3-azabicyclo[4.1.0]heptane-3-carboxylate

A solution of benzyl 7-(aminomethyl)-7-(5-methylisoxazol-3-yl)-3-azabicyclo[4.1.0]heptane-3-carboxylate (430 mg, 1.26 mmol) and TEA (246 uL, 1.76 mmol) in DCM (12 mL) was treated with (Boc)₂O (302 mg, 1.39 mmol) and the mixture was stirred at rt for 1 h. H₂O (5 mL) was added and mixture extracted with EtOAc (2×55 mL). The collected organics were washed with brine, dried over Na₂SO₄, and concentrated in vacuo to get a residue which was purified by flash chromatography (0-50% EtOAc in petroleum ether) to afford the title compound as a colorless oil (330 mg, 59%). LCMS (ES⁺) Method 2: m/z 464 (M+Na)⁺, RT 2.05 min.

### Step 4: tert-butyl ((7-(5-methylisoxazol-3-yl)-3-azabicyclo[4.1.0]heptan-7-yl)methyl)carbamate

A solution of benzyl 7-(((tert-butoxycarbonyl)amino)methyl)-7-(5-methylisoxazol-3-yl)-3-azabicyclo[4.1.0]heptane-3-carboxylate (320 mg, 0.720 mmol) in THF (7.0 mL) was treated with Pd/C (150 mg) and the resulting suspension was stirred under H₂ atmosphere at rt for 18 h. After N₂ fluxing, the catalyst was filtered on a pad of solka-floc which was washed with MeOH. After solvent removal the title compound was obtained as a colorless oil and used as crude. LCMS (ES⁺) Method 2: m/z 308 (M+H)⁺, RT 1.10 min.

### Intermediate 13: thieno[3,2-b]pyridin-7-ylboronic acid

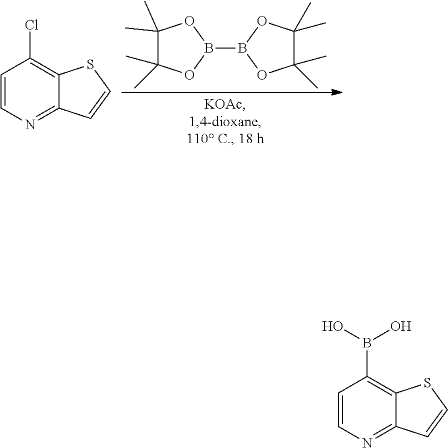

A suspension of B₂pin₂ (599 mg, 2.36 mmol), 7-chlorothieno[3,2-b]pyridine (200 mg, 1.18 mmol), Pd(dppf)Cl₂·DCM (48.7 mg, 0.060 mmol), and KOAc (347.1 mg, 3.54 mmol) in 1,4-dioxane (3.5 mL) was stirred at 110° C. for 4 h. After cooling, the mixture was filtered on a pad of solka-floc and the filtrate was washed with EtOAc. The organic phase was washed with brine, dried over Na₂SO₄ and concentrated in vacuo get the title compound which was used as crude. LCMS (ES⁺) Method 2: m/z 180 (M+H)⁺, RT 0.36 min.

### Intermediate 14: potassium thieno[3,2-b]pyridine-7-thiolate

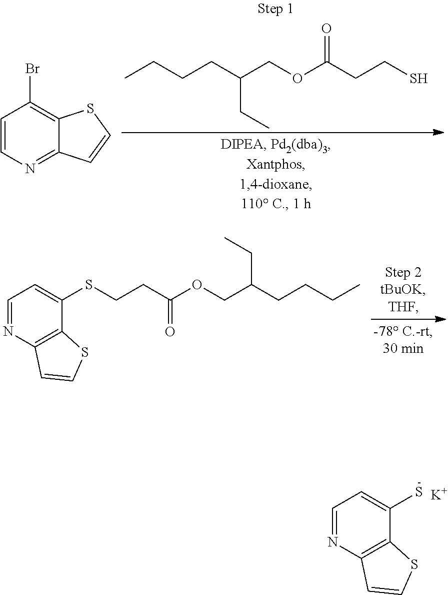

### Step 1: 2-ethylhexyl 3-(thieno[3,2-b]pyridin-7-ylthio)propanoate

A solution of 2-ethylhexyl 3-sulfanylpropanoate (76.5 mg, 0.350 mmol), DIPEA (90.6 mg, 0.700 mmol), Pd₂(dba)₃ (10.7 mg, 0.010 mmol) and XantPhos (6.8 mg, 0.010 mmol) in 1,4-dioxane (0.47 mL) was treated with 7-bromanylthieno[3,2-b]pyridine (50 mg, 0.230 mmol) and the mixture was stirred at 110° C. for 1 h. After cooling, the solvent was concentrated in vacuo and the residue purified by flash chromatography (0-50% EtOAc in petroleum ether) to afford the title compound as a yellow oil (48 mg, 58%). LCMS (ES⁺) Method 2: m/z 352 (M+H)⁺, RT 2.29 min.

### Step 2: potassium thieno[3,2-b]pyridine-7-thiolate

A solution of 2-ethylhexyl 3-(thieno[3,2-b]pyridin-7-ylthio)propanoate (48 mg, 0.140 mmol) in THF (0.47 mL) cooled to −78° C. was treated with potassium 2-methylpropan-2-olate (1 M in THF; 0.27 mL, 0.270 mmol). The reaction mixture was stirred at this temperature for 30 min then the solvent was concentrated under reduced pressure to obtain the title compound as a yellow solid which was used without further purification. LCMS (ES⁺) Method 2: m/z 167 (M+H)⁺, RT 0.61 min.

### Intermediate 15: 8-chloro-5-(4,4,5,5-tetramethyl-1,3,2-dioxaborolan-2-yl)quinoline

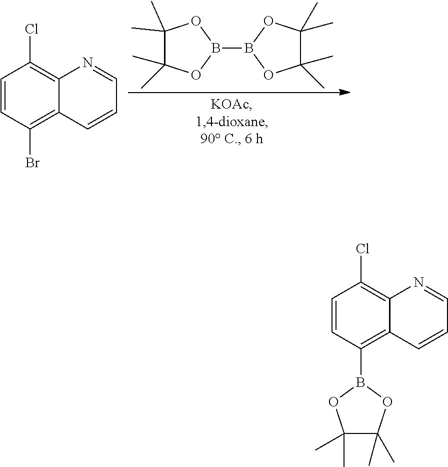

### Step 1: 8-chloro-5-(4,4,5,5-tetramethyl-1,3,2-dioxaborolan-2-yl)quinoline

A suspension of Pd(dppf)Cl₂-DCM (51 mg, 0.060 mmol), 5-bromanyl-8-chloranyl-quinoline (150 mg, 0.620 mmol), B₂pin₂ (346 mg, 1.36 mmol), and KOAc (182 mg, 1.86 mmol) in degassed 1,4-dioxane (2 mL) was stirred at 90° C. for 6 h. After cooling the mixture was diluted with EtOAc and washed with H₂O and brine. The organic phase was dried over Na₂SO₄ and concentrated in vacuo to afford the title compound which was used as crude. LCMS (ES⁺) Method 2: m/z 290 (M+H)⁺, RT 2.19 min.

The following compounds were synthesized according to the procedure reported for Intermediate 15 with the appropriate bromides starting materials.

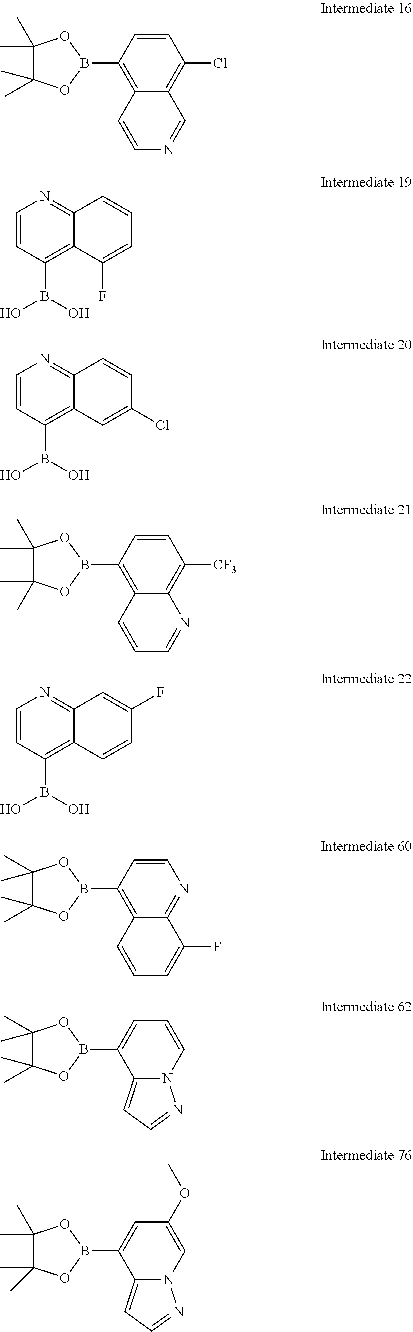

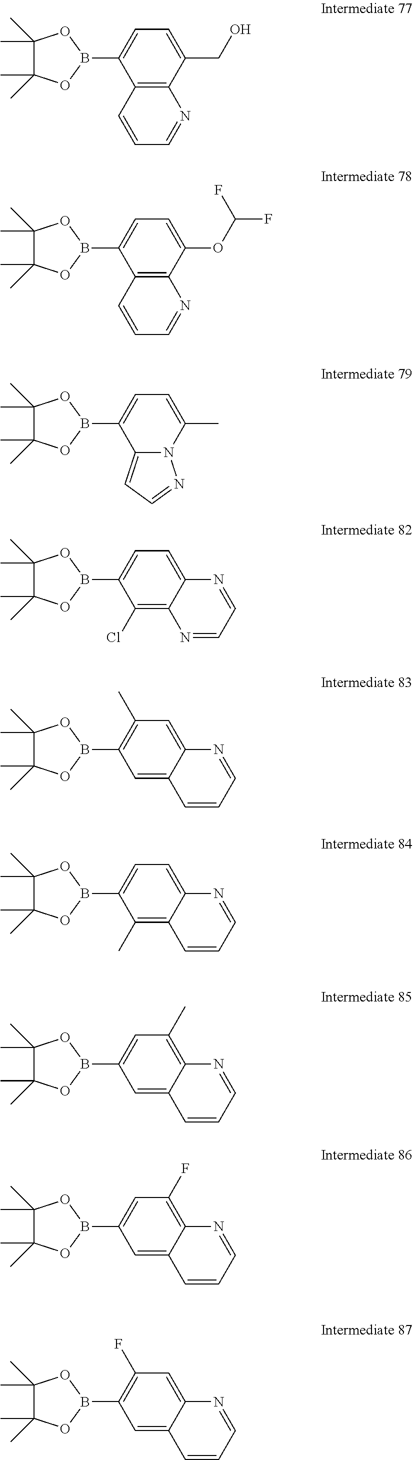

### Intermediate 17: (2-(bis(tert-butoxycarbonyl)amino)-3-chloropyridin-4-yl)boronic acid

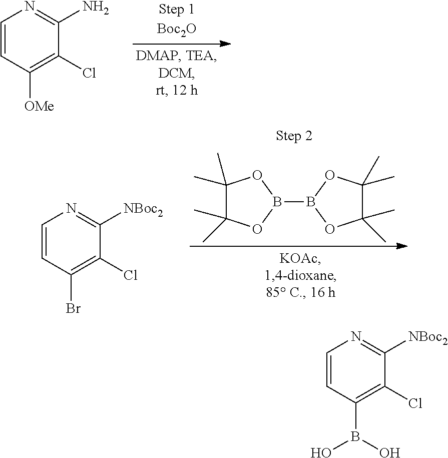

### Step 1: tert-butyl (4-bromo-3-chloropyridin-2-yl)(tert-butoxycarbonyl)carbamate

A solution of 4-bromo-3-chloropyridin-2-amine (106 mg, 0.510 mmol) and (Boc)₂O (557.6 mg, 2.55 mmol) in DCM (6 mL) was treated with DMAP (12.5 mg, 0.100 mmol) and TEA (0.21 mL, 1.53 mmol). The resulting mixture was stirred at rt for 12 h. H₂O (5 mL) was added dropwise and mixture was extracted with Et₂O (2×50 mL). Organic phase was washed with H₂O, brine, dried over Na₂SO₄ and concentrated in vacuo and the residue was purified by flash chromatography (0-50% EtOAc in petroleum ether) to afford the title compound as a white solid (184 mg, 88%). ¹H NMR (400 MHz, DMSO-d₆) δ 8.35 (d, J=4.0 Hz, 1H), 7.94 (d, J=4.1 Hz, 1H), 1.36 (s, 18H). LCMS (ES⁺) Method 2: m/z 407-409 (M+H)⁺, RT 2.26

### Step 2: (2-(bis(tert-butoxycarbonyl)amino)-3-chloropyridin-4-yl)boronic acid

A suspension of tert-butyl (4-bromo-3-chloropyridin-2-yl)(tert-butoxycarbonyl)carbamate (100 mg, 0.250 mmol), KOAc (48.2 mg, 0.490 mmol), Pd(dppf)Cl₂·DCM (20.3 mg, 0.020 mmol) and B₂pin₂ (68.5 mg, 0.270 mmol) in 1,4-dioxane (2.5 mL) was stirred at 85° C. for 16 h. After cooling, the mixture was filtered on a pad of solka-floc and the filtrate was washed with EtOAc. The organic phase was concentrated in vacuo to afford the title compound which was used as crude. LCMS (ES⁺) Method 2: m/z 373 (M+H)⁺, RT 1.55.

### Intermediate 18: (1S,6R,7S)-7-((((benzyloxy)carbonyl)amino)methyl)-7-(5-methylisoxazol-3-yl)-3-azabicyclo[4.1.0]heptan-3-ium chloride

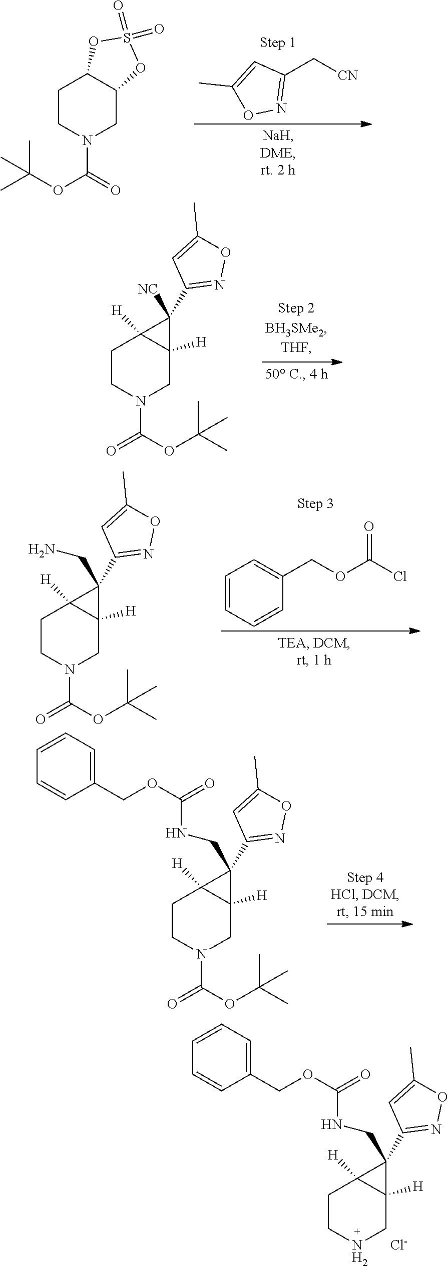

### Step 1: tert-butyl (1S,6R,7R)-7-cyano-7-(5-methylisoxazol-3-yl)-3-azabicyclo[4.1.0]heptane-3-carboxylate

A solution of NaH (60% in mineral oil; 558.5 mg, 13.96 mmol) in DME (13 mL) was treated with a solution of tert-butyl (3aR,7aS)-2,2-bis(oxidanylidene)-4,6,7,7a-tetrahydro-3aH-[1,3,2]dioxathiolo[4,5-c]pyridine-5-carboxylate (prepared using the procedure described in step 1 of Intermediate 39), 2-(5-methyl-1,2-oxazol-3-yl)ethanenitrile (721.4 mg, 5.91 mmol) and 15-crown-5 (107 uL, 0.540 mmol) in DME (13 mL) and the reaction mixture was stirred at rt for 2 h. H₂O (12 mL) was added and the mixture was extracted with EtOAc (2×90 mL). The collected organics were washed with brine, dried over Na₂SO₄, and concentrated in vacuo to get a residue which was purified by flash chromatography (0-40% EtOAc in petroleum ether) to afford the title compound as a yellow oil (1.1 g, 68%). LCMS (ES⁺) Method 2: m/z 326 (M+Na)⁺, RT 1.70 min.

### Step 2: tert-butyl (1S,6R,7S)-7-(aminomethyl)-7-(5-methylisoxazol-3-yl)-3-azabicyclo[4.1.0]heptane-3-carboxylate

A solution of tert-butyl (1S,6R,7R)-7-cyano-7-(5-methylisoxazol-3-yl)-3-azabicyclo[4.1.0]heptane-3-carboxylate (1.1 g, 3.63 mmol) in THF (45 mL) was treated with BMS (2M in THF; 6.35 mL, 12.69 mmol) and the resulting mixture was stirred at 50° C. for 4 h. After cooling, EtOH (15 mL) was added, and mixture stirred at reflux for 16 h. The volatiles were removed by reduced pressure and the title compound was used without further purification. LCMS (ES⁺) Method 2: m/z 308 (M+H)⁺, RT 1.15 min.

### Step 3: tert-butyl (1S,6R,7S)-7-((((benzyloxy)carbonyl)amino)methyl)-7-(5-methylisoxazol-3-yl)-3-azabicyclo[4.1.0]heptane-3-carboxylate

A solution of tert-butyl (1S,6R,7S)-7-(aminomethyl)-7-(5-methylisoxazol-3-yl)-3-azabicyclo[4.1.0]heptane-3-carboxylate (1.2 g, 3.9 mmol) in DCM (38 mL) cooled at 0° C. was treated with TEA (1.63 mL, 11.71 mmol), benzyl chloroformate (502 uL, 3.51 mmol) and the mixture was stirred at rt for 1 h. Brine was added and the mixture was extracted with EtOAc (2×100 mL). The collected organics were washed with H₂O, brine, dried over Na₂SO₄, and concentrated in vacuo to get a residue which was purified by flash chromatography (0-50% EtOAc in petroleum ether) to afford the title compound as a colorless oil (480 mg, 28%). LCMS (ES⁺) Method 2: m/z 464 (M+Na)⁺, RT 2.02 min.

### Step 4: (1S,6R,7S)-7-((((benzyloxy)carbonyl)amino)methyl)-7-(5-methylisoxazol-3-yl)-3-azabicyclo[4.1.0]heptan-3-ium chloride

A solution of tert-butyl (1S,6R,7S)-7-((((benzyloxy)carbonyl)amino)methyl)-7-(5-methylisoxazol-3-yl)-3-azabicyclo[4.1.0]heptane-3-carboxylate (480 mg, 1.09 mmol) in DCM (10 mL) was treated with HCl (4M in 1,4-dioxane; 10.87 mL, 43.49 mmol) and the solution stirred at rt for 15 min. The mixture was concentrated under reduced pressure to afford the title compound as a white solid which was used as a crude (410 mg, 100%). LCMS (ES⁺) Method 2: m/z 342 (M+H)⁺, RT 1.18 min.

The following compounds were synthesized according to the procedure reported for Intermediate 18 with the appropriate nitriles starting materials; for Intermediates 74, 89, 90, 91, 92, 93 (benzyl (2,5-dioxopyrrolidin-1-yl) carbonate in THF/H2O was used in step 3.

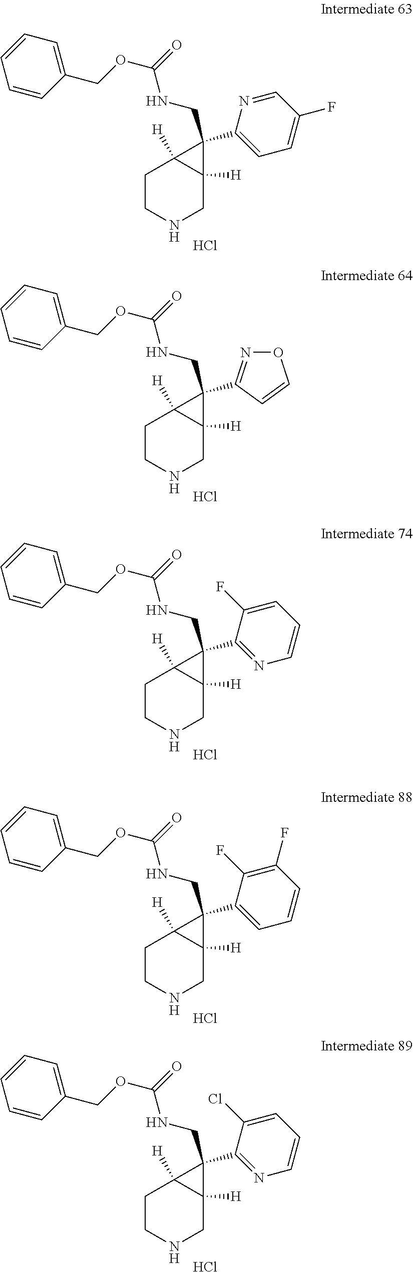

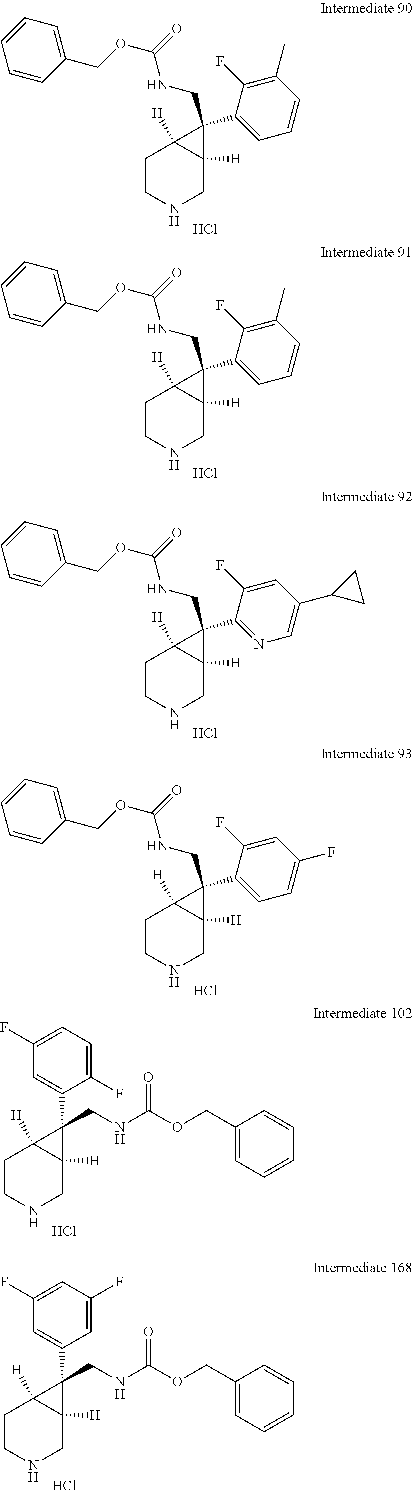

### Intermediate 23: 7-((((benzyloxy)carbonyl)amino)methyl)-7-(5-(methoxymethyl)isoxazol-3-yl)-3-azabicyclo[4.1.0]heptan-3-ium chloride

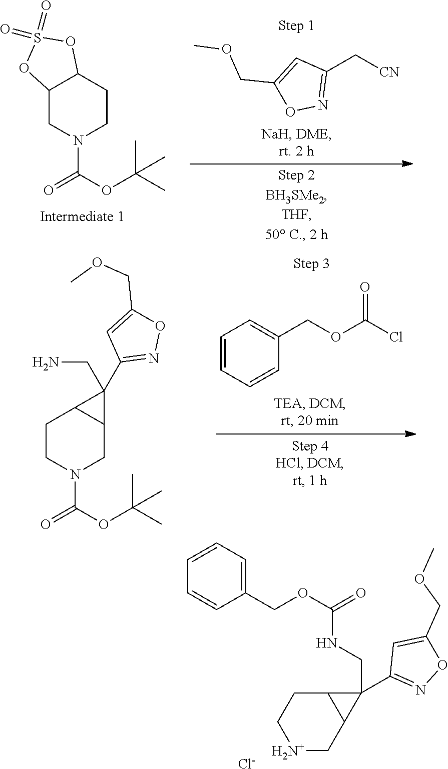 [SMILES: CC(C)(C)OC(=O)N1CCC2OS(=O)(=O)OC2C1 ; COCc1cc(CC#N)no1 ; COCc1cc(C2(CN)C3CCN(C(=O)OC(C)(C)C)CC32)no1 ; O=C(Cl)OCc1ccccc1 ; COCc1cc(C2(CNC(=O)OCc3ccccc3)C3CC[NH2+]CC32)no1 ; [Cl-]]

### Step 1: tert-butyl 7-cyano-7-(5-(methoxymethyl)isoxazol-3-yl)-3-azabicyclo[4.1.0]heptane-3-carboxylate

A solution of NaH (60% in mineral oil; 93.1 mg, 2.33 mmol) in DME (5.0 mL) was treated with a solution of tert-butyl tetrahydro-[1,3,2]dioxathiolo[4,5-c]pyridine-5(4H)-carboxylate 2,2-dioxide (Intermediate 1; 250 mg, 0.900 mmol), 2-(5-(methoxymethyl)isoxazol-3-yl)acetonitrile (150 mg, 0.980 mmol) and 15-crown-5 (17.9 uL, 0.090 mmol) in DME (5.0 mL) and the reaction mixture was stirred at rt for 2 h. H₂O (2 mL) was added and the mixture was extracted with EtOAc (3×50 mL). The collected organics were washed with brine, dried over Na₂SO₄, and concentrated in vacuo to get a residue which was purified by flash chromatography (0-60% EtOAc in petroleum ether) to afford the title compound as a yellow oil (185 mg, 62%). ¹H NMR (400 MHz, CDCl₃) δ 6.46 (s, 1H), 4.51 (s, 2H), 4.25 (d, J=16.0 Hz, 1H), 3.98-3.80 (m, 1H), 3.66 (dd, J=4.0, 16.0 Hz, 1H), 3.43 (s, 3H), 2.93-2.79 (m, 1H), 2.34-2.23 (m, 3H), 2.07-2.01 (m, 1H), 1.48 (s, 9H). LCMS (ES⁺) Method 2: m/z 356 (M+Na)⁺, RT 1.46 min.

### Step 2: tert-butyl 7-(aminomethyl)-7-(5-(methoxymethyl)isoxazol-3-yl)-3-azabicyclo[4.1.0]heptane-3-carboxylate

A solution of tert-butyl 7-cyano-7-(5-(methoxymethyl)isoxazol-3-yl)-3-azabicyclo[4.1.0]heptane-3-carboxylate (185 mg, 0.550 mmol) in THF (6 mL) was treated with BMS (2M in THF; 0.97 mL, 1.94 mmol) and the resulting mixture was stirred at 50° C. for 2 h. After cooling, EtOH (4 mL) was added, and the mixture stirred at reflux for 16 h. The volatiles were removed by reduced pressure and the title compound was used without further purification. LCMS (ES⁺) Method 2: m/z 338 (M+H)⁺, RT 0.97 min.

### Step 3: tert-butyl 7-((((benzyloxy)carbonyl)amino)methyl)-7-(5-(methoxymethyl)isoxazol-3-yl)-3-azabicyclo[4.1.0]heptane-3-carboxylate

A solution of tert-butyl 7-(aminomethyl)-7-(5-(methoxymethyl)isoxazol-3-yl)-3-azabicyclo[4.1.0]heptane-3-carboxylate (220 mg, 0.650 mmol) in DCM (6 mL) cooled at 0° C. was treated with TEA (0.27 mL, 1.96 mmol) and benzyl chloroformate (83.8 uL, 0.590 mmol) and the mixture was stirred at rt for 20 min. Brine was added and the mixture was extracted with EtOAc (2×20 mL). The collected organics were washed with H₂O, brine, dried over Na₂SO₄, and concentrated in vacuo to get a residue which was purified by flash chromatography (0-50% EtOAc in petroleum ether) to afford the title compound as a colorless oil (100 mg, 33%). LCMS (ES⁺) Method 2: m/z 494 (M+Na)⁺, RT 1.80 min.

### Step 4: 7-((((benzyloxy)carbonyl)amino)methyl)-7-(5-(methoxymethyl)isoxazol-3-yl)-3-azabicyclo[4.1.0]heptan-3-ium chloride

A solution of tert-butyl 7-((((benzyloxy)carbonyl)amino)methyl)-7-(5-(methoxymethyl)isoxazol-3-yl)-3-azabicyclo[4.1.0]heptane-3-carboxylate (100 mg, 0.210 mmol) in DCM (1 mL) was treated with HCl (4 M in 1,4-dioxane; 2.65 mL, 10.6 mmol) and the solution was stirred at rt for 1 h. The mixture was concentrated under reduced pressure to afford the title compound as a white solid which was used as a crude. LCMS (ES⁺) Method 2: m/z 372 (M+H)⁺, RT 0.99 min.

### Intermediate 24: 7-((((benzyloxy)carbonyl)amino)methyl)-7-(5-cyclopropylisoxazol-3-yl)-3-azabicyclo[4.1.0]heptan-3-ium chloride

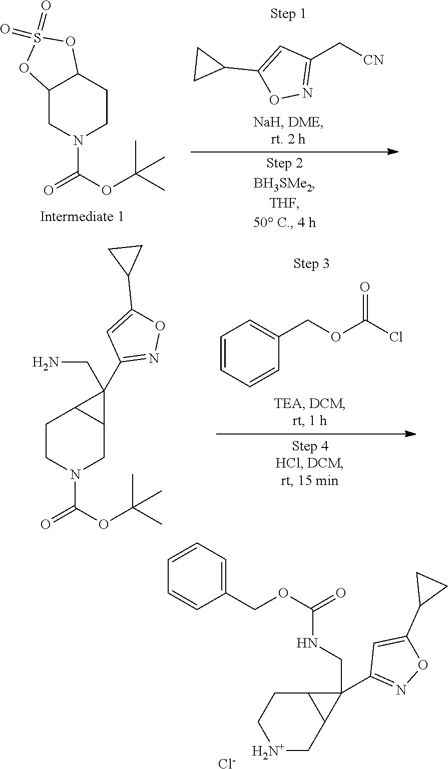 [SMILES: CC(C)(C)OC(=O)N1CCC2OS(=O)(=O)OC2C1 ; N#CCc1cc(C2CC2)on1 ; CC(C)(C)OC(=O)N1CCC2C(C1)C2(CN)c1cc(C2CC2)on1 ; O=C(Cl)OCc1ccccc1 ; O=C(NCC1(c2cc(C3CC3)on2)C2CC[NH2+]CC21)OCc1ccccc1 ; [Cl-]]

### Step 1: tert-butyl 7-cyano-7-(5-cyclopropylisoxazol-3-yl)-3-azabicyclo[4.1.0]heptane-3-carboxylate

A solution of NaH (60% in mineral oil; 111.7 mg, 2.79 mmol) in DME (5.0 mL) was treated with a solution of tert-butyl tetrahydro-[1,3,2]dioxathiolo[4,5-c]pyridine-5(4H)-carboxylate 2,2-dioxide (Intermediate 1; 300 mg, 1.07 mmol), 2-(5-cyclopropylisoxazol-3-yl)acetonitrile (175.1 mg, 1.18 mmol) and 15-crown-5 (21.5 uL, 0.110 mmol) in DME (5.0 mL) and the reaction mixture was stirred at rt for 2 h. H₂O (2 mL) was added and the mixture was extracted with EtOAc (2×50 mL). The collected organics were washed with brine, dried over Na₂SO₄, and concentrated in vacuo to get a residue which was purified by flash chromatography (0-50% EtOAc in petroleum ether) to afford the title compound as a yellow oil (220 mg, 62%). LCMS (ES⁺) Method 2: m/z 352 (M+Na)⁺, RT 1.88 min.

### Step 2: tert-butyl 7-(aminomethyl)-7-(5-cyclopropylisoxazol-3-yl)-3-azabicyclo[4.1.0]heptane-3-carboxylate

A solution of tert-butyl 7-cyano-7-(5-cyclopropylisoxazol-3-yl)-3-azabicyclo[4.1.0]heptane-3-carboxylate (220 mg, 0.670 mmol) in THF (6.5 mL) was treated with BMS (2M in THF; 1.17 mL, 2.34 mmol) and the resulting mixture was stirred at 50° C. for 4 h. After cooling, EtOH (4 mL) was added, and mixture stirred at reflux for 16 h. The volatiles were removed by reduced pressure and the title compound was used without further purification. LCMS (ES⁺) Method 2: m/z 334 (M+H)⁺, RT 1.09 min.

### Step 3: tert-butyl 7-((((benzyloxy)carbonyl)amino)methyl)-7-(5-cyclopropylisoxazol-3-yl)-3-azabicyclo[4.1.0]heptane-3-carboxylate

A solution of tert-butyl 7-(aminomethyl)-7-(5-cyclopropylisoxazol-3-yl)-3-azabicyclo[4.1.0]heptane-3-carboxylate (270 mg, 0.810 mmol) in DCM (7 mL) cooled at 0° C. was treated with TEA (0.34 mL, 2.43 mmol) and benzyl chloroformate (104 uL, 0.730 mmol) and the mixture was stirred at rt for 1 h. Brine was added and the mixture extracted with EtOAc (2×40 mL). The collected organics were washed with H₂O, brine, dried over Na₂SO₄, and concentrated in vacuo to get a residue which was purified by flash chromatography (0-40% EtOAc in petroleum ether) to afford the title compound as a colorless oil (83 mg, 22%). LCMS (ES⁺) Method 2: m/z 490 (M+Na)⁺, RT 1.96 min.

### Step 4: 7-((((benzyloxy)carbonyl)amino)methyl)-7-(5-cyclopropylisoxazol-3-yl)-3-azabicyclo[4.1.0]heptan-3-ium chloride

A solution of tert-butyl 7-((((benzyloxy)carbonyl)amino)methyl)-7-(5-cyclopropylisoxazol-3-yl)-3-azabicyclo[4.1.0]heptane-3-carboxylate (85 mg, 0.180 mmol) in DCM (1 mL) was treated with HCl (4M in 1,4-dioxane; 2.27 mL, 9.09 mmol) and the solution was stirred at rt for 15 min. The mixture was concentrated under reduced pressure to afford the title compound as a white solid which was used as a crude. LCMS (ES⁺) Method 2: m/z 368 (M+H)⁺, RT 1.15 min.

### Intermediate 25: 7-((((benzyloxy)carbonyl)amino)methyl)-7-(3-fluoropyridin-2-yl)-3-azabicyclo[4.1.0]heptan-3-ium chloride

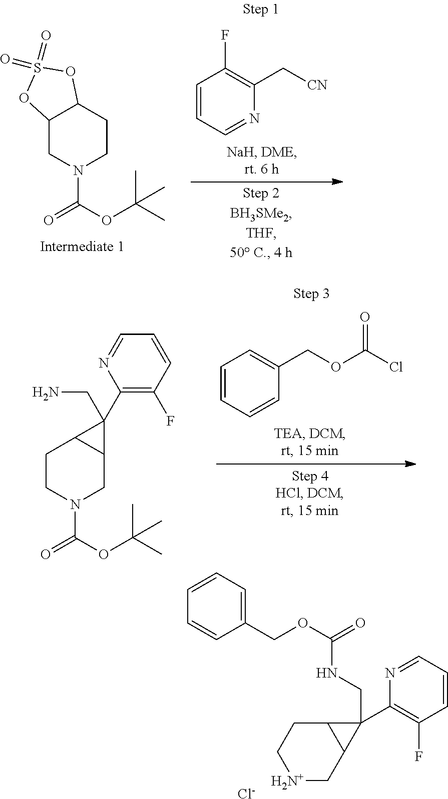 [SMILES: CC(C)(C)OC(=O)N1CCC2OS(=O)(=O)OC2C1 ; N#CCc1ncccc1F ; CC(C)(C)OC(=O)N1CCC2C(C1)C2(CN)c1ncccc1F ; O=C(Cl)OCc1ccccc1 ; O=C(NCC1(c2ncccc2F)C2CC[NH2+]CC21)OCc1ccccc1 ; [Cl-]]

### Step 1: tert-butyl 7-cyano-7-(3-fluoropyridin-2-yl)-3-azabicyclo[4.1.0]heptane-3-carboxylate

A solution of NaH (60% in mineral oil; 111.7 mg, 2.79 mmol) in DME (3.5 mL) was treated with a solution of tert-butyl tetrahydro-[1,3,2]dioxathiolo[4,5-c]pyridine-5(4H)-carboxylate 2,2-dioxide (Intermediate 1; 300 mg, 1.07 mmol), 2-(3-fluoropyridin-2-yl)acetonitrile (160.8 mg, 1.18 mmol) and 15-crown-5 (21.3 uL, 0.110 mmol) in DME (3.5 mL) and the reaction mixture was stirred at rt for 6 h. H₂O (4 mL) was added and the mixture was extracted with EtOAc (2×50 mL). The collected organics were washed with brine, dried over Na₂SO₄, and concentrated in vacuo to get a residue which was purified by flash chromatography (0-60% EtOAc in petroleum ether) to afford the title compound as a yellow oil (73 mg, 21%). LCMS (ES⁺) Method 2: m/z 340 (M+Na)⁺, RT 1.70 min.

### Step 2: tert-butyl 7-(aminomethyl)-7-(3-fluoropyridin-2-yl)-3-azabicyclo[4.1.0]heptane-3-carboxylate

A solution of tert-butyl 7-cyano-7-(3-fluoropyridin-2-yl)-3-azabicyclo[4.1.0]heptane-3-carboxylate (73 mg, 0.230 mmol) in THF (3.0 mL) was treated with BMS (2M in THF; 402.0 uL, 0.810 mmol) and the resulting mixture was stirred at 50° C. for 4 h. After cooling, EtOH (5 mL) was added and mixture stirred at reflux for 6 h. The volatiles were removed by reduced pressure and the title compound was used without further purification. LCMS (ES⁺) Method 2: m/z 322 (M+H)⁺, RT 1.15 min.

### Step 3: tert-butyl 7-((((benzyloxy)carbonyl)amino)methyl)-7-(3-fluoropyridin-2-yl)-3-azabicyclo[4.1.0]heptane-3-carboxylate

A solution of tert-butyl 7-(aminomethyl)-7-(3-fluoropyridin-2-yl)-3-azabicyclo[4.1.0]heptane-3-carboxylate (85 mg, 0.260 mmol) in DCM (3 mL) cooled at 0° C. was treated with TEA (111 uL, 0.790 mmol) and benzyl chloroformate (34 uL, 0.240 mmol) and mixture was stirred at rt for 15 min. Brine was added and the mixture was extracted with EtOAc (2×20 mL). The collected organics were washed with H₂O, brine, dried over Na₂SO₄, and concentrated in vacuo to get a residue which was purified by flash chromatography (0-70% EtOAc in petroleum ether) to afford the title compound as a colorless oil (25 mg, 21%). LCMS (ES⁺) Method 2: m/z 478 (M+Na)⁺, RT 1.70 min.

### Step 4: 7-((((benzyloxy)carbonyl)amino)methyl)-7-(3-fluoropyridin-2-yl)-3-azabicyclo[4.1.0]heptan-3-ium chloride

A solution of tert-butyl 7-((((benzyloxy)carbonyl)amino)methyl)-7-(3-fluoropyridin-2-yl)-3-azabicyclo[4.1.0]heptane-3-carboxylate (23 mg, 0.050 mmol) in DCM (0.8 mL) was treated with HCl (4M in 1,4-dioxane; 0.5 mL, 2.02 mmol) and the solution stirred at rt for 15 min. The mixture was concentrated under reduced pressure to afford the title compound as a white solid which was used as a crude. LCMS (ES⁺) Method 2: m/z 356 (M+H)⁺, RT 0.98 min.

### Intermediate 26: 7-((((benzyloxy)carbonyl)amino)methyl)-7-(5-fluoropyridin-2-yl)-3-azabicyclo[4.1.0]heptan-3-ium chloride

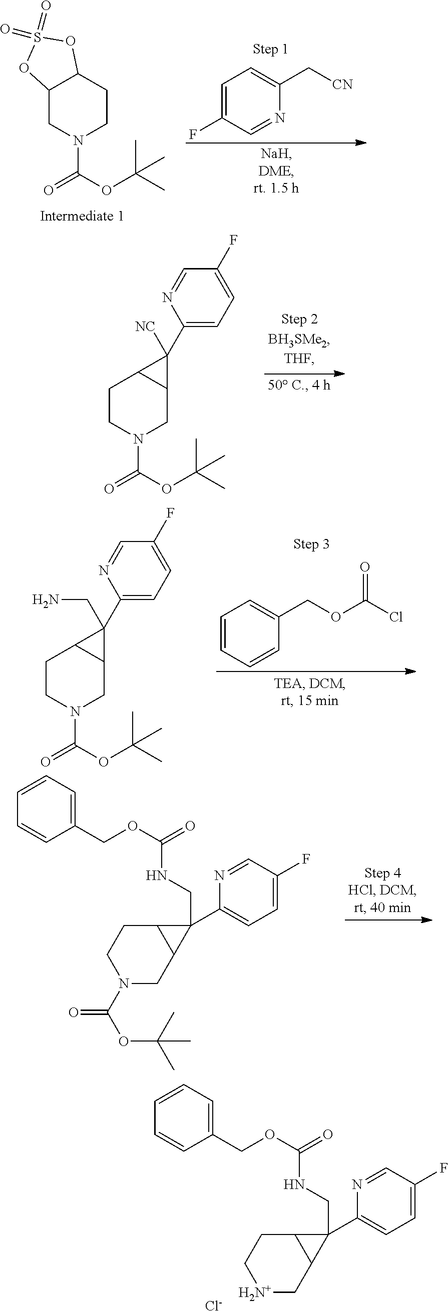

### Step 1: tert-butyl 7-cyano-7-(5-fluoropyridin-2-yl)-3-azabicyclo[4.1.0]heptane-3-carboxylate

A solution of NaH (60% in mineral oil; 111.7 mg, 2.79 mmol) in DME (5 mL) was treated with a solution of tert-butyl tetrahydro-[1,3,2]dioxathiolo[4,5-c]pyridine-5(4H)-carboxylate 2,2-dioxide (Intermediate 1; 300 mg, 1.07 mmol), 2-(5-fluoropyridin-2-yl)acetonitrile (160.8 mg, 1.18 mmol) and 15-crown-5 (21 uL, 0.110 mmol) in DME (5 mL) and the reaction mixture was stirred at rt for 1.5 h. H₂O (2 mL) was added and the mixture was extracted with EtOAc (2×50 mL). The collected organics were washed with brine, dried over Na₂SO₄, and concentrated in vacuo to get a residue which was purified by flash chromatography (0-50% EtOAc in petroleum ether) to afford the title compound as a yellow oil (275 mg, 73%). LCMS (ES⁺) Method 2: m/z 218 (M+H-Boc)⁺, RT 1.92 min.

### Step 2: tert-butyl 7-(aminomethyl)-7-(5-fluoropyridin-2-yl)-3-azabicyclo[4.1.0]heptane-3-carboxylate

A solution of tert-butyl 7-cyano-7-(5-fluoropyridin-2-yl)-3-azabicyclo[4.1.0]heptane-3-carboxylate (275 mg, 0.870 mmol) in THF (6.5 mL) was treated with BMS (2M in THF; 1.52 mL, 3.03 mmol) and the resulting mixture was stirred at 50° C. for 4 h. After cooling, EtOH (4 mL) was added, and mixture stirred at reflux for 16 h. The volatiles were removed by reduced pressure and the title compound was used without further purification. LCMS (ES⁺) Method 2: m/z 322 (M+H)⁺, RT 1.08 min.

### Step 3: tert-butyl 7-((((benzyloxy)carbonyl)amino)methyl)-7-(5-fluoropyridin-2-yl)-3-azabicyclo[4.1.0]heptane-3-carboxylate

A solution of tert-butyl 7-(aminomethyl)-7-(5-fluoropyridin-2-yl)-3-azabicyclo[4.1.0]heptane-3-carboxylate (275 mg, 0.390 mmol) in DCM (3 mL) cooled at 0° C. was treated with TEA (0.16 mL, 1.16 mmol) and benzyl chloroformate (49.0 uL, 0.350 mmol) and the mixture was stirred at rt for 1 h. Brine was added and mixture extracted with EtOAc (2×100 mL). The collected organics were washed with H₂O, brine, dried over Na₂SO₄, and concentrated in vacuo to get a residue which was purified by flash chromatography (0-65% EtOAc in petroleum ether) to afford the title compound as a yellow oil (52 mg, 29%). LCMS (ES⁺) Method 2: m/z 478 (M+Na)⁺, RT 1.90 min.

### Step 4: 7-((((benzyloxy)carbonyl)amino)methyl)-7-(5-fluoropyridin-2-yl)-3-azabicyclo[4.1.0]heptan-3-ium chloride

A solution of tert-butyl 7-((((benzyloxy)carbonyl)amino)methyl)-7-(5-fluoropyridin-2-yl)-3-azabicyclo[4.1.0]heptane-3-carboxylate (25 mg, 0.050 mmol) in DCM (10 mL) was treated with HCl (4M in 1,4-dioxane; 0.55 mL, 2.2 mmol) and the solution stirred at rt for 40 min. The mixture was concentrated under reduced pressure to afford the title compound as a white solid which was used as a crude. LCMS (ES⁺) Method 2: m/z 356 (M+H)⁺, RT 1.05 min.

### Intermediate 27: 3,3-difluoro-1-methyl-4-(4,4,5,5-tetramethyl-1,3,2-dioxaborolan-2-yl)indolin-2-one

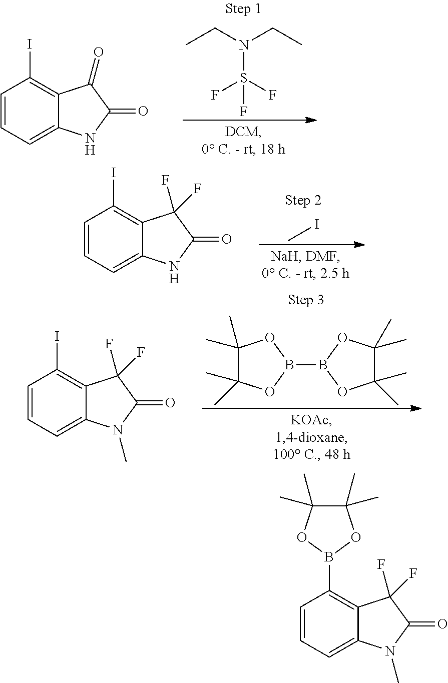

### Step 1: 3,3-difluoro-4-iodoindolin-2-one

A solution of 4-iodoindoline-2,3-dione (600 mg, 2.2 mmol) in DCM (8 mL) cooled at 0° C. was treated with N,N-diethyl-1,1,1-trifluoro-4-sulfanamine (1.06 g, 6.59 mmol). The resulting mixture was stirred at rt for 18 h and then treated dropwise with NaHCO₃ sat. sol. in H₂O (1.5 g in 8 mL) under vigorous stirring. The layers were separated, and the aqueous phase was extracted with DCM (2×40 mL). The collected organics were washed with brine, dried over Na₂SO₄, and concentrated in vacuo to get a residue which was purified by flash chromatography (0-50% EtOAc in petroleum ether) to afford the title compound as a purple solid (330 mg, 51%). ¹H NMR (400 MHz, DMSO-d₆) δ 11.29 (bs, 1H), 7.53 (d, J=8.0 Hz, 1H), 7.24 (t, J=8.1 Hz, 1H), 6.98 (d, J=8.0 Hz, 1H). LCMS (ES⁺) Method 2: m/z 296 (M+H)⁺, RT 1.61 min

### Step 2: 3,3-difluoro-4-iodo-1-methylindolin-2-one

A solution of 3,3-difluoro-4-iodoindolin-2-one (100 mg, 0.340 mmol) in DMF (2 mL) cooled at 0° C. was treated with NaH (60% in mineral oil; 27 mg, 0.680 mmol). The resulting mixture was stirred at rt for 30 min then was treated with Mel (42 uL, 0.680 mmol) and stirred at rt for 2 h. H₂O (2 mL) was added dropwise, and mixture extracted with EtOAc (2×20 mL). Organic layer was separated and washed with brine, dried over Na₂SO₄, and concentrated in vacuo to obtain a residue which was purified by flash chromatography (0-50% EtOAc in petroleum ether) to afford the title compound as a yellow solid (83 mg, 79%). ¹H NMR (400 MHz, CDCl₃) δ 7.54 (d, J=8.0 Hz, 1H), 7.18 (t, J=8.1 Hz, 1H), 6.86 (d, J=8.0 Hz, 1H), 3.21 (s, 3H). LCMS (ES⁺) Method 2: m/z 310 (M+H)⁺, RT 1.75 min

### Step 3: 3,3-difluoro-1-methyl-4-(4,4,5,5-tetramethyl-1,3,2-dioxaborolan-2-yl)indolin-2-one

A suspension of Pd(dppf)Cl₂·DCM (16.1 mg, 0.020 mmol), 3,3-difluoro-4-iodo-1-methylindolin-2-one (60 mg, 0.190 mmol), B₂pin₂ (108.5 mg, 0.430 mmol), and KOAc (57.2 mg, 0.580 mmol) in degassed 1,4-dioxane (1 mL) was stirred at 100° C. for 48 h. After cooling, the mixture was filtered on a pad of solka-floc and the filtrate was washed with EtOAc. The organic phase was concentrated in vacuo to obtain a residue which was purified by flash chromatography (0-50% EtOAc in petroleum ether) to afford the title compound as a yellow oil (45 mg, 75%). LCMS (ES⁺) Method 2: m/z 310 (M+H)⁺, RT 2.00 min

### Intermediate 28: 7-((((benzyloxy)carbonyl)amino)methyl)-7-phenyl-3-azabicyclo[4.1.0]heptan-3-ium chloride

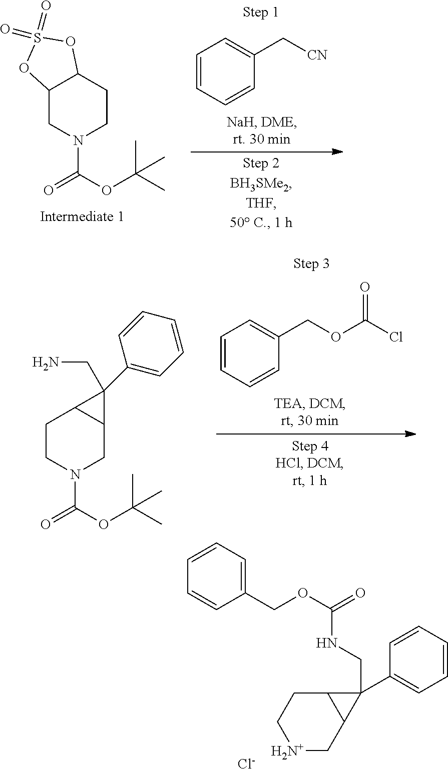

### Step 1: tert-butyl 7-cyano-7-phenyl-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 560 mg, 2.01 mmol) and 2-phenylacetonitrile (258 mg, 2.21 mmol) were dissolved in dry DMF (4.5 mL) and the resulting solution was added dropwise over a period of 30 min to a suspension of NaH (60% in mineral oil; 200 mg, 5, 01 mmol) e) in dry DMF (2.8 mL) cooled to 0° C. under N₂ atmosphere. The reaction mixture was stirred at 0° C. for 10 min then at rt for 30 min. The reaction was quenched by addition of brine then diluted with EtOAc. The organic layer was washed with brine (×2) and H₂O (×2), dried over Na₂SO₄, filtered, and concentrated in vacuo. The residue was purified by flash chromatography (10-30% EtOAc in petroleum ether) to afford the title compound as a light-yellow oil (452 mg, 76%). ¹H NMR (300 MHz, CDCl₃) δ 7.42-7.33 (m, 2H), 7.32-7.24 (m, 3H), 4.15 (m, 1H), 4.04-3.64 (m, 2H), 3.16-2.73 (m, 1H), 2.25 (m, 1H), 2.09 (m, 1H), 2.00 (m, 1H), 1.92 (m, 1H), 1.51 (s, 9H); LCMS (ES⁺) Method 1A: m/z 299 (M+H)⁺, RT 2.17 min.

### Step 2: tert-butyl 7-(aminomethyl)-7-phenyl-3-azabicyclo[4.1.0]heptane-3-carboxylate

Tert-butyl 7-cyano-7-phenyl-3-azabicyclo[4.1.0]heptane-3-carboxylate (120 mg, 0.40 mmol) and CoCl₂ (208 mg, 1.6 mmol) were dissolved in MeOH (5 mL) in a pressure flask. The dark solution was cooled to 0° C. NaBH₄ (456 mg, 12 mmol) was added portionwise. Then, the flask was sealed, and the reaction mixture was vigorously stirred for 12 h at rt. The solvent was removed in vacuo to obtain the crude compound, that was used in the next step without further purification. LCMS (ES⁺) Method 1A: m/z 303 (M+H)⁺, RT 1.37 min.

### Step 3: tert-butyl 7-((((benzyloxy)carbonyl)amino)methyl)-7-phenyl-3-azabicyclo[4.1.0]heptane-3-carboxylate

To a solution of crude tert-butyl 7-(aminomethyl)-7-phenyl-3-azabicyclo[4.1.0]heptane-3-carboxylate (0.40 mmol) in DCM (5 mL), cooled to 0° C., benzyl chloroformate (0.12 mL, 0.80 mmol) and TEA (0.09 mL, 0.60 mmol) were sequentially added. The reaction mixture was stirred at 0° C. for 30 min. H₂O was added and the phases were separated. Then the organic layer was washed with 5% citric acid solution (2 mL×2), dried over Na₂SO₄, filtered and concentrated under reduced pressure. The resulting crude was purified by flash chromatography (0-10% EtOAc in DCM) affording the title compound as a colourless oil (100 mg, 57% over two steps). LCMS (ES⁺) Method 1A: m/z 437 (M+H)⁺, RT 2.44 min.

### Step 4: 7-((((benzyloxy)carbonyl)amino)methyl)-7-phenyl-3-azabicyclo[4.1.0]heptan-3-ium chloride

Tert-butyl 7-((((benzyloxy)carbonyl)amino)methyl)-7-phenyl-3-azabicyclo[4.1.0]heptane-3-carboxylate (35 mg, 0.08 mmol) was dissolved in DCM (1 mL) and treated with HCl (4N in 1,4-dioxane, 1 mL, 4.0 mmol). The solution was stirred at rt for 1.5 h, then solvent was removed in vacuo to afford the title compound that was used in the next step without further purification (35 mg, 99%). LCMS (ES⁺) Method 1A: m/z 337 (M+H)⁺, RT 1.42 min.

### Intermediate 29: benzyl ((7-(pyridin-2-yl)-3-azabicyclo[4.1.0]heptan-7-yl)methyl)carbamate dihydrochloride

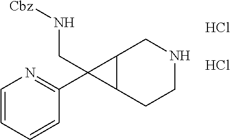 [SMILES: O=C(NCC1(c2ccccn2)C2CCNCC21)OCc1ccccc1 ; Cl ; Cl]

### Step 1: tert-butyl 7-cyano-7-(pyridin-2-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 520 mg, 1.86 mmol), and 2-(pyridin-2-yl)acetonitrile (242 mg, 2.05 mmol) were dissolved in dry DME (3.7 mL), and the resulting solution was added dropwise over a period of 30 min to a suspension of NaH (60% in mineral oil; 186 mg, 4.6 mmol) in DME (6.7 mL) at rt under N₂ atmosphere. The reaction mixture was stirred at rt for 3 h, then quenched by addition of H₂O and extracted with EtOAc (×2). The combined organic layers were dried over Na₂SO₄, filtered, and concentrated in vacuo. The residue was purified by flash chromatography (C18; 10-40% MeCN/H₂O+0.1% TFA) to afford the title compound as a yellow oil (225 mg, 40%). ¹H NMR (300 MHz, CDCl₃) δ 8.45 (m, 1H), 7.80-7.64 (m, 2H), 7.16 (m, 1H), 4.22 (d, J=14.6 Hz, 1H), 4.09-3.78 (m, 1H), 3.72 (dd, J=14.6, 5.3 Hz, 1H), 3.11-2.76 (m, 1H), 2.50-2.33 (m, 2H), 2.25 (m, 1H), 2.12-1.98 (m, 1H), 1.51 (s, 9H); LCMS (ES⁺) Method 1A: m/z 300 (M+H)⁺, RT 1.93 min.

### Step 2: tert-butyl 7-(aminomethyl)-7-(pyridin-2-yl)-3-azabicyclo[4.1.0]heptane-3-carboxylate

Tert-Butyl 7-cyano-7-(pyridin-2-yl)-3-azabicyclo[4.1.0]heptane-3-carboxylate (150 mg, 0.50 mmol) and CoCl₂ (357 mg, 1.5 mmol) were dissolved in MeOH (7 mL) in a pressure flask. The dark solution was cooled to 0° C. and NaBH₄ (568 mg, 15 mmol) was added portionwise. The flask was sealed, and the reaction mixture was vigorously stirred for 12 h at rt. The solvent was removed in vacuo to obtain the crude compound that was used in the next step without further purification. LCMS (ES⁺) Method 1A: m/z 304 (M+H)⁺, RT 1.02 min.

### Step 3: tert-butyl 7-((((benzyloxy)carbonyl)amino)methyl)-7-(pyridin-2-yl)-3-azabicyclo[4.1.0]heptane-3-carboxylate

Tert-butyl 7-(aminomethyl)-7-(pyridin-2-yl)-3-azabicyclo[4.1.0]heptane-3-carboxylate from previous step (0.50 mmol) was reacted as described in the synthesis of Intermediate 28 step 3. The residue was purified by preparative HPLC (5-45% MeCN/H₂O+0.1% TFA) affording the title compound as a yellow solid (45 mg, 16% over two steps). LCMS (ES⁺) Method 1A: m/z 438 (M+H)⁺, RT 1.52 min.

### Step 4: benzyl ((7-(pyridin-2-yl)-3-azabicyclo[4.1.0]heptan-7-yl)methyl)carbamate dihydrochloride

Tert-butyl 7-((((benzyloxy)carbonyl)amino)methyl)-7-(pyridin-2-yl)-3-azabicyclo[4.1.0]heptane-3-carboxylate (22 mg, 0.04 mmol) was dissolved in DCM (1 mL) and treated with HCl (4N in 1,4-dioxane, 1 mL, 4.0 mmol). The solution was stirred at rt for 1.5 h, then the solvent was removed in vacuo to afford the title compound (18 mg, 99%). LCMS (ES⁺) Method 1A: m/z 338 (M+H)⁺, RT 0.85 min.

### Intermediate 30: 7-((((benzyloxy)carbonyl)amino)methyl)-7-(pyrimidin-2-yl)-3-azabicyclo[4.1.0]heptan-3-ium chloride

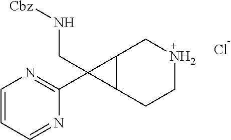 [SMILES: O=C(NCC1(c2ncccn2)C2CCNCC21)OCc1ccccc1 ; Cl]

### Step 1: tert-butyl 7-cyano-7-(pyrimidin-2-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 350 mg, 1.25 mmol), and 2-(pyrimidin-2-yl)acetonitrile (246 mg, 2.07 mmol) were dissolved in dry DME (2.5 mL) and the resulting solution was added dropwise over a period of 30 min to a suspension of NaH (60% in mineral oil; 125 mg, 3.1 mmol) in DME (1.8 mL) at rt under N₂ atmosphere. The reaction mixture was stirred at rt for 3 h, then quenched by addition of H₂O and extracted with EtOAc (×2). Combined organic layers were dried over Na₂SO₄, filtered, and concentrated in vacuo. The residue was purified by flash chromatography (C18; 10-40% MeCN/H₂O+0.1% TFA) to afford the title compound as an orange foam (150 mg, 40%). ¹H NMR (300 MHz, CDCl₃) δ 8.67 (d, J=4.9 Hz, 2H), 7.18 (t, J=4.9 Hz, 1H), 4.18 (m, 1H), 3.99 (m, 1H), 3.75 (m, 1H), 3.29-2.76 (m, 1H), 2.58-2.19 (m, 3H), 2.09 (m, 1H), 1.51 (s, 9H); LCMS (ES⁺) Method 1A: m/z 301 (M+H)⁺, RT 1.73 min.

### Step 2: tert-butyl 7-(aminomethyl)-7-(pyrimidin-2-yl)-3-azabicyclo[4.1.0]heptane-3-carboxylate

Tert-butyl 7-cyano-7-(pyrimidin-2-yl)-3-azabicyclo[4.1.0]heptane-3-carboxylate (85 mg, 0.28 mmol) and CoCl₂ (109 mg, 0.84 mmol) were dissolved in MeOH (0.07 M) in a pressure flask. The dark solution was cooled to 0° C. and NaBH₄ (317 mg, 8.4 mmol) was added portionwise. The flask was sealed, and the reaction mixture was vigorously stirred for 12 h at rt. The solvent was removed in vacuo to obtain the crude compound that was used in the next step without further purification. LCMS (ES⁺) Method 1A: m/z 305 (M+H)⁺, RT 1.16 min.

### Step 3: tert-butyl 7-((((benzyloxy)carbonyl)amino)methyl)-7-(pyrimidin-2-yl)-3-azabicyclo[4.1.0]heptane-3-carboxylate

Tert-butyl 7-(aminomethyl)-7-(pyrimidin-2-yl)-3-azabicyclo[4.1.0]heptane-3-carboxylate (0.28 mmol) was reacted as described in the synthesis of Intermediate 28 step 3. The residue was purified by flash chromatography (10-50% EtOAc in petroleum ether) affording the title compound as a colourless oil (18 mg, 15% over two steps). ¹H NMR (300 MHz, CDCl₃) δ 8.59 (d, J=4.9 Hz, 2H), 7.47-7.29 (m, 5H), 7.07 (t, J=4.9 Hz, 1H), 6.29-5.75 (m, 1H), 5.11 (m, 2H), 3.89 (m, 4H), 3.65 (m, 1H), 2.88 (m, 1H), 2.21-2.01 (m, 2H), 1.90 (m, 1H), 1.63 (m, 1H), 1.51 (s, 9H); LCMS (ES⁺) Method 1A: m/z 439 (M+H)⁺, RT 2.23 min.

### Step 4: 7-((((benzyloxy)carbonyl)amino)methyl)-7-(pyrimidin-2-yl)-3-azabicyclo[4.1.0]heptan-3-ium

Tert-butyl 7-((((benzyloxy)carbonyl)amino)methyl)-7-(pyrimidin-2-yl)-3-azabicyclo[4.1.0]heptane-3-carboxylate (18 mg, 0.04 mmol) was dissolved in DCM (1 mL) and treated with HCl (4N in 1,4-dioxane, 1 mL, 4.0 mmol). The solution was stirred at rt for 1.5 h, then solvent was removed in vacuo to afford the title compound to give the title compound (15 mg; 97%). LCMS (ES⁺) Method 1A: m/z 339 (M+H)⁺, RT 1.24 min.

### Intermediate 31: 7-((((benzyloxy)carbonyl)amino)methyl)-7-(thiophen-3-yl)-3-azabicyclo[4.1.0]heptan-3-ium chloride

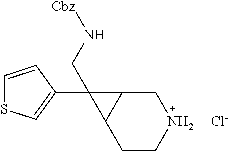 [SMILES: O=C(NCC1(c2ccsc2)C2CCNCC21)OCc1ccccc1 ; [Cl-]]

### Step 1: tert-butyl 7-cyano-7-(thiophen-3-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 350 mg, 1.25 mmol) and 2-(thiophen-3-yl)acetonitrile (170 mg, 1.38 mmol) were dissolved in dry DMF (2.7 ml) and the resulting solution was added dropwise over a period of 30 min to a suspension of NaH (60% in mineral oil; 124 mg, 3.1 mmol) in dry DMF (0.7 mL) cooled to 0° C. under N₂ atmosphere. The reaction mixture was stirred at 0° C. for 10 min then at rt for 30 min. The reaction was quenched by addition of brine then diluted with EtOAc. The organic layer was washed with brine (×2) and H₂O (×2), dried over Na₂SO₄, filtered, and concentrated in vacuo. The residue was purified by flash chromatography (0-5% EtOAc in petroleum ether) to afford the title compound as a yellow oil (140 mg, 37%). ¹H NMR (300 MHz, CDCl₃) δ 7.33 (m, 1H), 7.17 (m, 1H), 6.90 (m, 1H), 4.14 (m, 1H), 4.04-3.61 (m, 2H), 3.13-2.71 (m, 1H), 2.22 (m, 1H), 2.11-1.75 (m, 3H), 1.50 (s, 9H); LCMS (ES⁺) Method 1A: m/z 305 (M+H)⁺, RT 2.12 min.

### Step 2: tert-butyl 7-(aminomethyl)-7-(thiophen-3-yl)-3-azabicyclo[4.1.0]heptane-3-carboxylate

Tert-butyl 7-cyano-7-(thiophen-3-yl)-3-azabicyclo[4.1.0]heptane-3-carboxylate (65 mg, 0.21 mmol) and CoCl₂ (82 mg, 0.63 mmol) were dissolved in MeOH (3 mL) in a pressure flask. The dark solution was cooled to 0° C. and NaBH₄ (238 mg, 6.3 mmol) was added portionwise. The flask was sealed, and the reaction mixture was vigorously stirred for 12 h at rt. The solvent was removed in vacuo to obtain crude compound that was used in the next step without further purification. LCMS (ES⁺) Method 1A: m/z 309 (M+H)⁺, RT 1.32 min.

### Step 3: tert-butyl 7-((((benzyloxy)carbonyl)amino)methyl)-7-(thiophen-3-yl)-3-azabicyclo[4.1.0]heptane-3-carboxylate

Tert-butyl 7-(aminomethyl)-7-(thiophen-3-yl)-3-azabicyclo[4.1.0]heptane-3-carboxylate (0.21 mmol) was reacted as described in the synthesis of Intermediate 28 step 3. The residue was purified by preparative HPLC (30-75% MeCN/H₂O+0.1% HCOOH) affording the title compound as a white solid (5 mg, 5% over two steps). LCMS (ES⁺) Method 1A: m/z 443 (M+H)⁺, RT 2.41 min.

### Step 4: 7-((((benzyloxy)carbonyl)amino)methyl)-7-(thiophen-3-yl)-3-azabicyclo[4.1.0]heptan-3-ium chloride

Tert-butyl 7-((((benzyloxy)carbonyl)amino)methyl)-7-(thiophen-3-yl)-3-azabicyclo[4.1.0]heptane-3-carboxylate (5 mg, 0.01 mmol) was dissolved in DCM (1 mL) and treated with 0.5 mL of HCl (4N in 1,4-dioxane). LCMS (ES⁺) Method 1A: m/z 343 (M+H)⁺, RT 1.36 min.

### Intermediate 32: 7-((((benzyloxy)carbonyl)amino)methyl)-7-(4-methylthiazol-2-yl)-3-azabicyclo[4.1.0]heptan-3-ium chloride

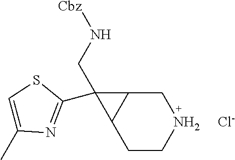 [SMILES: Cc1csc(C2(CNC(=O)OCc3ccccc3)C3CCNCC32)n1 ; [Cl-]]

### Step 1: tert-butyl 7-cyano-7-(4-methylthiazol-2-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 154 mg, 0.55 mmol), 15-crown-5 (11 μL, 0.055 mmol) and 2-(4-methylthiazol-2-yl)acetonitrile (84 mg, 0.61 mmol) were dissolved in dry DME (1.1 mL) and the resulting solution was added dropwise over a period of 30 min to a suspension of NaH (60% in mineral oil; 34 mg, 1.43 mmol) in DME (0.8 mL) at rt under N₂ atmosphere. The reaction mixture was stirred at rt for 3 h, then quenched by addition of H₂O and extracted with EtOAc (×2). The combined organic layers were dried over Na₂SO₄, filtered, and concentrated in vacuo. The residue was purified by flash chromatography (0-20% EtOAc in petroleum ether) to afford the title compound as a red oil (100 mg, 57%). ¹H NMR (300 MHz, CDCl₃) δ 6.76 (br d, J=0.9 Hz, 1H), 4.25 (d, J=14.8 Hz, 1H), 4.10-3.75 (m, 1H), 3.69 (dd, J=14.8, 5.0 Hz, 1H), 3.05-2.70 (m, 1H), 2.55-2.40 (m, 2H), 2.38 (d, J=0.9 Hz, 3H), 2.33-2.15 (m, 1H), 2.13-1.98 (m, 1H), 1.50 (s, 9H); LCMS (ES⁺) Method 1A: m/z 320 (M+H)⁺, RT 2.00 min.

### Step 2: tert-butyl 7-(aminomethyl)-7-(4-methylthiazol-2-yl)-3-azabicyclo[4.1.0]heptane-3-carboxylate

To a solution of tert-butyl 7-cyano-7-(4-methylthiazol-2-yl)-3-azabicyclo[4.1.0]heptane-3-carboxylate (120 mg, 0.38 mmol) in dry THF (2.4 mL), with BMS (2M in THF; 0.8 mL, 1.52 mmol) was added and the reaction mixture was stirred at 50° C. for 1 h. Additional BMS (2M in THF; 0.4 mL, 0.8 mmol) was added and the reaction mixture was stirred at 50° C. for 2 h. The reaction was quenched by addition of EtOH (0.08 M) and the resulting solution was stirred at 70° C. for 3 h. The solvent was removed in vacuo to obtain the crude title compound, which was used in the next step without further purification. LCMS (ES⁺) Method 1A: m/z 324 (M+H)⁺, RT 1.29 min.

### Step 3: tert-butyl 7-((((benzyloxy)carbonyl)amino)methyl)-7-(4-methylthiazol-2-yl)-3-azabicyclo[4.1.0]heptane-3-carboxylate

Tert-butyl 7-(aminomethyl)-7-(4-methylthiazol-2-yl)-3-azabicyclo[4.1.0]heptane-3-carboxylate (0.38 mmol) was prepared according to the procedure described for Intermediate 28 step 3. The residue was purified by flash chromatography (0-100% EtOAc in DCM) affording the title compound as a light-yellow oil (29 mg, 17% over two steps). ¹H NMR (300 MHz, CDCl₃) δ 7.43-7.30 (m, 5H), 6.70 (s, 1H), 5.86-5.42 (m, 1H), 5.11 (m, 2H), 4.03 (d, J=14.8 Hz, 1H), 3.96-3.69 (m, 3H), 3.64-3.46 (m, 1H), 2.95-2.53 (m, 1H), 2.40 (s, 3H), 2.22-2.03 (m, 1H), 2.01-1.77 (m, 2H), 1.70 (m, 1H), 1.48 (s, 9H); LCMS (ES⁺) Method 1A: m/z 458 (M+H)⁺, RT 2.10 min.

### Step 4: 7-((((benzyloxy)carbonyl)amino)methyl)-7-(4-methylthiazol-2-yl)-3-azabicyclo[4.1.0]heptan-3-ium chloride (Intermediate 32)

Tert-butyl 7-((((benzyloxy)carbonyl)amino)methyl)-7-(4-methylthiazol-2-yl)-3-azabicyclo[4.1.0]heptane-3-carboxylate (29 mg, 0.06 mmol) was dissolved in DCM (1 mL) and treated with 1 mL of HCl (4N in 1,4-dioxane). The solution was stirred at rt for 1.5 h, then the solvent was removed in vacuo to afford the title compound (29 mg; 99%). LCMS (ES⁺) Method 1A: m/z 358 (M+H)⁺, RT 1.26 min.

### Intermediate 33: 7-((((benzyloxy)carbonyl)amino)methyl)-7-(1-methyl-1H-pyrazol-3-yl)-3-azabicyclo[4.1.0]heptan-3-ium chloride

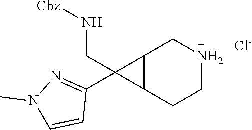 [SMILES: Cn1ccc(C2(CNC(=O)OCc3ccccc3)C3CCNCC32)n1 ; [Cl-]]

### Step 1: tert-butyl 7-cyano-7-(1-methyl-1H-pyrazol-3-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 100 mg, 0.36 mmol) and 2-(1-methyl-1H-pyrazol-3-yl)acetonitrile (47 mg, 0.39 mmol) were dissolved in dry DMF (0.8 mL) and the resulting solution was added dropwise over a period of 30 min to a suspension of NaH (60% in mineral oil; 37 mg, 0.9 mmol) in dry DMF (0.2 mL) cooled to 0° C. under N₂ atmosphere. The reaction mixture was stirred at 0° C. for 10 min then at rt for 30 min. The reaction was quenched by addition of brine then diluted with EtOAc. The organic layer was washed with brine (×2) and H₂O (×2), dried over Na₂SO₄, filtered, and concentrated in vacuo. The residue was purified by flash chromatography (0-100% EtOAc in petroleum ether) to afford the title compound as yellow oil (24 mg, 22%). ¹H NMR (300 MHz, CDCl₃) δ 7.22-7.15 (m, 1H), 6.23 (d, J=2.2 Hz, 1H), 4.04 (br d, J=13.7 Hz, 1H), 3.93-3.51 (m, 5H), 2.97-2.70 (m, 1H), 2.19-1.81 (m, 4H), 1.40 (s, 9H); LCMS (ES⁺) Method 1A: m/z 303 (M+H)⁺, RT 1.72 min.

### Step 2: tert-butyl 7-(aminomethyl)-7-(1-methyl-1H-pyrazol-3-yl)-3-azabicyclo[4.1.0]heptane-3-carboxylate

To a solution of tert-butyl 7-cyano-7-(1-methyl-1H-pyrazol-3-yl)-3-azabicyclo[4.1.0]heptane-3-carboxylate (23 mg, 0.08 mmol) in dry THF (0.5 mL), BMS (2M in THF; 0.16 mL, 0.32 mmol) was added and the reaction mixture was stirred at 50° C. for 1 h. Additional BMS (2M in THF; 0.08 mL, 0.16 mmol) was added and the reaction mixture was stirred at 50° C. for 2 h. The reaction was quenched by addition of EtOH (1 mL) and the resulting solution was stirred at 70° C. for 3 h. The solvent was removed in vacuo to obtain the crude title compound, which was used in the next step without further purification. LCMS (ES⁺) Method 1A: m/z 307 (M+H)⁺, RT 1.21 min.

### Step 3: tert-butyl 7-((((benzyloxy)carbonyl)amino)methyl)-7-(1-methyl-1H-pyrazol-3-yl)-3-azabicyclo[4.1.0]heptane-3-carboxylate

Tert-butyl 7-(aminomethyl)-7-(1-methyl-1H-pyrazol-3-yl)-3-azabicyclo[4.1.0]heptane-3-carboxylate (0.08 mmol) was reacted as described in the synthesis of Intermediate 28 step 3. The residue was purified by flash chromatography (0-100% EtOAc/DCM) affording the title compound as a pale-yellow oil (17 mg, 49% over two steps). ¹H NMR (300 MHz, CDCl₃) δ 7.34-7.11 (m, 6H), 5.89 (br s, 1H), 5.50-5.25 (m, 1H), 5.14-4.90 (m, 2H), 3.93-3.39 (m, 8H), 2.80-2.52 (m, 1H), 2.06-1.91 (m, 1H), 1.78-1.54 (m, 3H), 1.39 (s, 9H); LCMS (ES⁺) Method 1A: m/z 441 (M+H)⁺, RT 2.05 min.

### Step 4: 7-((((benzyloxy)carbonyl)amino)methyl)-7-(1-methyl-1H-pyrazol-3-yl)-3-azabicyclo[4.1.0]heptan-3-ium chloride

Tert-butyl 7-((((benzyloxy)carbonyl)amino)methyl)-7-(1-methyl-1H-pyrazol-3-yl)-3-azabicyclo[4.1.0]heptane-3-carboxylate (17 mg, 0.039 mmol) was dissolved in DCM (1 mL) and treated with 1 mL of HCl (4N in 1,4-dioxane). The solution was stirred at rt for 1.5 h, then the solvent was removed in vacuo to give the title compound (17 mg; yield). LCMS (ES⁺) Method 1A: m/z 341 (M+H)⁺, RT 1.19 min.

### Intermediate 34: 7-((((benzyloxy)carbonyl)amino)methyl)-7-(1-methyl-1H-pyrazol-5-yl)-3-azabicyclo[4.1.0]heptan-3-ium chloride

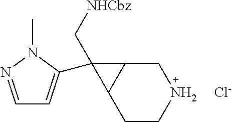 [SMILES: Cn1nccc1C1(CNC(=O)OCc2ccccc2)C2CCNCC21 ; [Cl-]]

### Step 1: tert-butyl 7-cyano-7-(I-methyl-1H-pyrazol-5-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 100 mg, 0.36 mmol) and 2-(1-methyl-1H-pyrazol-5-yl)acetonitrile (50 mg, 0.39 mmol) were dissolved in dry DMF (0.8 mL) and the resulting solution was added dropwise over a period of 30 min to a suspension of NaH (60% in mineral oil; 37 mg, 0.9 mmol) in dry DMF (0.2 mL) cooled to 0° C. under N₂ atmosphere. The reaction mixture was stirred at 0° C. for 10 min then at rt for 30 min. The reaction was quenched by addition of brine then diluted with EtOAc. The organic layer was washed with brine (×2) and H₂O (×2), dried over Na₂SO₄, filtered, and concentrated in vacuo. The residue was purified by flash chromatography (0-100% EtOAc in petroleum ether) to afford the title compound as a yellow oil (44 mg, 41%). ¹H NMR (300 MHz, CDCl₃) δ 7.31 (s, 1H), 6.01 (s, 1H), 4.20 (br s, 1H), 3.92-3.60 (m, 5H), 2.93-2.70 (m, 1H), 2.17 (br s, 1H), 2.07-1.72 (m, 3H), 1.40 (s, 9H); LCMS (ES⁺) Method 1A: m/z 303 (M+H)⁺, RT 1.66 min.

### Step 2: Tert-butyl 7-(aminomethyl)-7-(1-methyl-1H-pyrazol-5-yl)-3-azabicyclo[4.1.0]heptane-3-carboxylate

To a solution of tert-butyl 7-cyano-7-(1-methyl-1H-pyrazol-5-yl)-3-azabicyclo[4.1.0]heptane-3-carboxylate (40 mg, 0.13 mmol) in dry THF (0.8 mL), BMS (2M in THF; 0.26 mL, 0.52 mmol) was added and the reaction mixture was stirred at 50° C. for 1 h. Additional BMS (2M in THF; 0.13 mL, 0.26 mmol) was added and the reaction mixture was stirred at 50° C. for 2 h. The reaction was quenched by addition of EtOH (0.08M) and the resulting solution was stirred at 70° C. for 3 h. The solvent was removed in vacuo to obtain crude title compound, which was used in the next step without further purification. LCMS (ES⁺) Method 1A m/z 307 (M+H)⁺, RT 1.10 min.

### Step 3: tert-butyl 7-((((benzyloxy)carbonyl)amino)methyl)-7-(1-methyl-1H-pyrazol-5-yl)-3-azabicyclo[4.1.0]heptane-3-carboxylate

The compound was prepared according to the procedure described in synthesis of Intermediate 28 step 3, starting from tert-butyl 7-(aminomethyl)-7-(1-methyl-1H-pyrazol-5-yl)-3-azabicyclo[4.1.0]heptane-3-carboxylate (0.13 mmol). The residue was purified by flash chromatography (0-100% EtOAc in DCM) affording the title compound as a light-yellow oil (30 mg, 52% over 2 steps). ¹H NMR (300 MHz, CDCl₃) δ 7.34-7.17 (m, 6H), 5.97 (s, 1H), 4.97-4.76 (m, 3H), 4.21-3.20 (m, 9H), 2.57 (br s, 1H), 2.13-1.91 (m, 1H), 1.83-1.47 (m, 2H), 1.39 (s, 9H); LCMS (ES⁺) Method 1A: m/z 441 (M+H)⁺, RT 1.93 min.

### Step 4: 7-((((benzyloxy)carbonyl)amino)methyl)-7-(I-methyl-1H-pyrazol-5-yl)-3-azabicyclo[4.1.0]heptan-3-ium chloride

Tert-butyl 7-((((benzyloxy)carbonyl)amino)methyl)-7-(1-methyl-1H-pyrazol-5-yl)-3-azabicyclo[4.1.0]heptane-3-carboxylate (30 mg, 0.07 mmol) was dissolved in DCM (1 mL) and treated with 1 mL of HCl (4N in 1,4-dioxane). The solution was stirred at rt for 1.5 h, then solvent was removed in vacuo to give the title compound (30 mg). LCMS (ES⁺) Method 1A: m/z 341 (M+H)⁺, RT 1.07 min.

### Intermediate 35: 7-((((benzyloxy)carbonyl)amino)methyl)-7-(isoxazol-3-yl)-3-azabicyclo[4.1.0]heptan-3-ium chloride

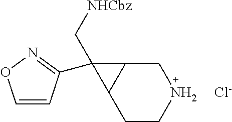 [SMILES: O=C(NCC1(c2ccon2)C2CCNCC21)OCc1ccccc1 ; [Cl-]]

### Step 1: tert-butyl 7-cyano-7-(isoxazol-3-yl)-3-azabicyclo[4.1.0]heptane-3-carboxylate

Tert-butyl tetrahydro-[1,3,2]dioxathiolo[4,5-c]pyridine-5(4H)-carboxylate 2,2-dioxide (Intermediate 1; 100 mg, 0.36 mmol), 15-crown-5 (7 μL, 0.055 mmol) and 2-(isoxazol-3-yl)acetonitrile (42 mg, 0.39 mmol) were dissolved in dry DME (0.7 mL), and the resulting solution was added dropwise over a period of 30 min to a suspension of NaH (60% in mineral oil; 37 mg, 0.94 mmol) in DME (0.5 mL) at rt under N₂ atmosphere. The reaction mixture was stirred at rt for 3 h, then quenched by addition of H₂O and extracted with EtOAc (×2). Combined organic layers were dried over Na₂SO₄, filtered, and concentrated in vacuo. The residue was purified by flash chromatography (0-100% EtOAc in petroleum ether) to afford the title compound as a yellow oil (48 mg, 46%). ¹H NMR (300 MHz, CDCl₃) δ 8.30 (d, J=1.6 Hz, 1H), 6.47 (d, J=1.6 Hz, 1H), 4.20 (br d, J=14.7 Hz, 1H), 3.97-3.72 (m, 1H), 3.58 (dd, J=14.7, 5.0 Hz, 1H), 2.92-2.62 (m, 1H), 2.30-2.09 (m, 3H), 2.05-1.89 (m, 1H), 1.40 (s, 9H); LCMS (ES⁺) Method 1A: m/z 290 (M+H)⁺, RT 1.76 min.

### Step 2: tert-butyl 7-(aminomethyl)-7-(isoxazol-3-yl)-3-azabicyclo[4.1.0]heptane-3-carboxylate

To a solution of tert-butyl 7-cyano-7-(isoxazol-3-yl)-3-azabicyclo[4.1.0]heptane-3-carboxylate (48 mg, 0.17 mmol) in dry THF (1 mL), BMS (2M in THF; 0.34 mL, 0.68 mmol) was added and the reaction mixture was stirred at 50° C. for 1 h. Additional BMS (2M in THF; 0.17 mL, 0.34 mmol) was added and the reaction mixture was stirred at 50° C. for 2 h. The reaction was quenched by addition of EtOH (2 mL) and the resulting solution was stirred at 70° C. for 3 h. Solvent was removed in vacuo to obtain the crude title compound, which was used in the next step without further purification. LCMS (ES⁺) Method 1A: m/z 294 (M+H)⁺, RT 1.13 min.

### Step 3: tert-butyl 7-((((benzyloxy)carbonyl)amino)methyl)-7-(isoxazol-3-yl)-3-azabicyclo[4.1.0]heptane-3-carboxylate

Tert-butyl 7-(aminomethyl)-7-(isoxazol-3-yl)-3-azabicyclo[4.1.0]heptane-3-carboxylate (0.16 mmol) was prepared according to the procedure described for Intermediate 28 step 3. The residue was purified by flash chromatography (0-50% EtOAc in DCM) affording the title compound as a pale-yellow oil (25 mg; 36% over 2 steps). Method 1A: m/z 428 (M+H)⁺, RT 2.14 min.

### Step 4: 7-((((benzyloxy)carbonyl)amino)methyl)-7-(isoxazol-3-yl)-3-azabicyclo[4.1.0]heptan-3-ium chloride

Tert-butyl 7-((((benzyloxy)carbonyl)amino)methyl)-7-(isoxazol-3-yl)-3-azabicyclo[4.1.0]heptane-3-carboxylate (25 mg, 0.06 mmol) was dissolved in DCM (1 mL) and treated with an excess of HCl 4N in 1,4-dioxane. The solution was stirred at rt for 1.5 h, then the solvent was removed in vacuo to give the title compound (25 mg; 99%). LCMS (ES⁺) Method 1A: m/z 328 (M+H)⁺, RT 1.21 min.

### Intermediate 36: 7-((1,3-dioxoisoindolin-2-yl)methyl)-7-(4-methylthiazol-2-yl)-3-azabicyclo[4.1.0]heptan-3-ium chloride

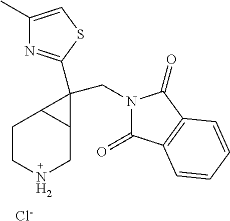 [SMILES: Cc1csc(C2(CN3C(=O)c4ccccc4C3=O)C3CCNCC32)n1 ; [Cl-]]

### Step 1: tert-butyl 7-((1,3-dioxoisoindolin-2-yl)methyl)-7-(4-methylthiazol-2-yl)-3-azabicyclo[4.1.0]heptane-3-carboxylate

To a solution of tert-butyl 7-(aminomethyl)-7-(4-methylthiazol-2-yl)-3-azabicyclo[4.1.0]heptane-3-carboxylate (prepared as described in the synthesis of Intermediate 32 step 2; 185 mg, 0.57 mmol) and phthalic anhydride (102 mg, 0.69 mmol) in dry toluene (6.5 mL), TEA (0.12 mL, 0.86 mmol) was added and the solution was stirred at 100° C. for 8 h. The reaction mixture was cooled to rt, then diluted with EtOAc and washed with 5% citric acid solution (×2), NaHCO₃ sat. sol. (×2) and brine (×2). The organic layer was dried over Na₂SO₄, filtered, and concentrated in vacuo. The resulting crude was purified by flash chromatography (0-40% EtOAc in DCM) affording the title compound as an off-white foam (173 mg, 67%). ¹H NMR (300 MHz, CDCl₃) δ 7.80 (m, 2H), 7.69 (m, 2H), 6.58 (s, 1H), 4.52 (m, 1H), 4.20 (d, J=14.4 Hz, 1H), 4.11-3.80 (m, 2H), 3.52 (dd, J=14.4, 6.1 Hz, 1H), 2.69 (m, 1H), 2.44-2.15 (m, 2H), 2.11 (s, 3H), 1.97 (m, 1H), 1.71 (m, 1H), 1.47 (s, 9H); LCMS (ES⁺) Method 1A: m/z 454 (M+H)⁺, RT 2.03 min.

### Step 2: 7-((1,3-dioxoisoindolin-2-yl)methyl)-7-(4-methylthiazol-2-yl)-3-azabicyclo[4.1.0]heptan-3-ium chloride (Intermediate 36)

Tert-butyl 7-((1,3-dioxoisoindolin-2-yl)methyl)-7-(4-methylthiazol-2-yl)-3-azabicyclo[4.1.0]heptane-3-carboxylate (173 mg, 0.38 mmol) was dissolved in DCM (1 mL) and treated with an excess of HCl 4N in 1,4-dioxane. The solution was stirred at rt for 1.5 h, then solvent was removed in vacuo to afford the title compound (169 mg; 99%). LCMS (ES⁺) Method 2: m/z 354 (M+H)⁺, RT 1.08 min.

### Intermediate 37: 7-((1,3-dioxoisoindolin-2-yl)methyl)-7-(thiophen-3-yl)-3-azabicyclo[4.1.0]heptan-3-ium chloride

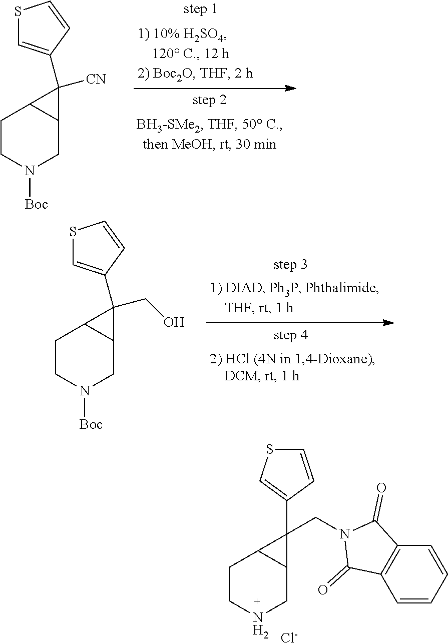 [SMILES: CC(C)(C)OC(=O)N1CCC2C(C1)C2(C#N)c1ccsc1 ; CC(C)(C)OC(=O)N1CCC2C(C1)C2(CO)c1ccsc1 ; O=C1c2ccccc2C(=O)N1CC1(c2ccsc2)C2CCNCC21 ; [Cl-]]

### Step 1: 3-(tert-butoxycarbonyl)-7-(thiophen-3-yl)-3-azabicyclo[4.1.0]heptane-7-carboxylic acid

Tert-butyl 7-cyano-7-(thiophen-3-yl)-3-azabicyclo[4.1.0]heptane-3-carboxylate (prepared as described in the synthesis of Intermediate 31 step 1 (140 mg, 0.46 mmol)) was dissolved in 10% aq. H₂SO₄ solution (5 mL) and the reaction mixture was stirred for 15 h at 130° C. The reaction was then cooled to 0° C. and treated with a solution of ION NaOH until pH=10. The reaction mixture was diluted with THF (5 mL), (Boc)₂O (351 mg, 1.61 mmol) was added and was vigorously stirred at rt for 2.5 h, then diluted with DCM and the layers were separated. The aqueous phase was treated with 6N HCl solution until pH=2 and extracted with EtOAc (×2). Collected organic layers were dried over Na₂SO₄, filtered, and evaporated to afford the title compound as a beige solid, which was used in the next step without further purification (139 mg, 93%). LCMS (ES⁺) Method 1A: m/z 324 (M+H)⁺, RT 1.91 min.

### Step 2: tert-butyl 7-(hydroxymethyl)-7-(thiophen-3-yl)-3-azabicyclo[4.1.0]heptane-3-carboxylate

To a solution of 3-(tert-butoxycarbonyl)-7-(thiophen-3-yl)-3-azabicyclo[4.1.0]heptane-7-carboxylic acid (100 mg, 0.31 mmol) in dry THF (2 mL), BMS (2M in THF; 0.54 mL, 1.08 mmol) was added and the reaction mixture was stirred at rt for 12 h. The reaction was quenched by addition of MeOH (4 mL), stirred at rt for 30 min then solvent was concentrated in vacuo. The resulting crude was purified by flash chromatography (0-30% EtOAc in DCM) to afford the title compound as a colourless oil (78 mg, 82%). LCMS (ES⁺) Method 1A: m/z 310 (M+H)⁺, RT 1.94 min.

### Step 3: tert-butyl 7-((1,3-dioxoisoindolin-2-yl)methyl)-7-(thiophen-3-yl)-3-azabicyclo[4.1.0]heptane-3-carboxylate

To a solution of tert-butyl 7-(hydroxymethyl)-7-(thiophen-3-yl)-3-azabicyclo[4.1.0]heptane-3-carboxylate (110 mg, 0.36 mmol), phthalimide (63 mg, 0.43 mmol) and PPh₃ (158.5 mg, 0.60 mmol) in dry THF (3.4 mL), DIAD (0.12 mL, 0.60 mmol) was added dropwise at rt. The resulting yellow solution was stirred at rt for 1 h, then solvent was removed in vacuo and the residue was purified by flash chromatography (10-20% EtOAc in petroleum ether) to afford the title compound as a white foam (145 mg, 93%). LCMS (ES⁺) Method 1A: m/z 439 (M+H)⁺, RT 2.41 min.

### Step 4: 2-((7-(thiophen-3-yl)-3-azabicyclo[4.1.0]heptan-7-yl)methyl)isoindoline-1,3-dione

Tert-butyl 7-((1,3-dioxoisoindolin-2-yl)methyl)-7-(thiophen-3-yl)-3-azabicyclo[4.1.0]heptane-3-carboxylate (145 mg, 0.33 mmol) was dissolved in DCM (2 mL) and treated with HCl (4N in dioxane 2.07 mL, 8.27 mmol). The solution was stirred at rt for 1 h, then the solvent was removed in vacuo to afford the crude title compound that was used in the next step without further purification (145 mg; 99%). LCMS (ES⁺) Method 1A: m/z 339 (M+H)⁺, RT 1.28 min.

### Intermediate 38: 2-((3-azabicyclo[4.1.0]heptan-7-yl)methyl)isoindoline-1,3-dione

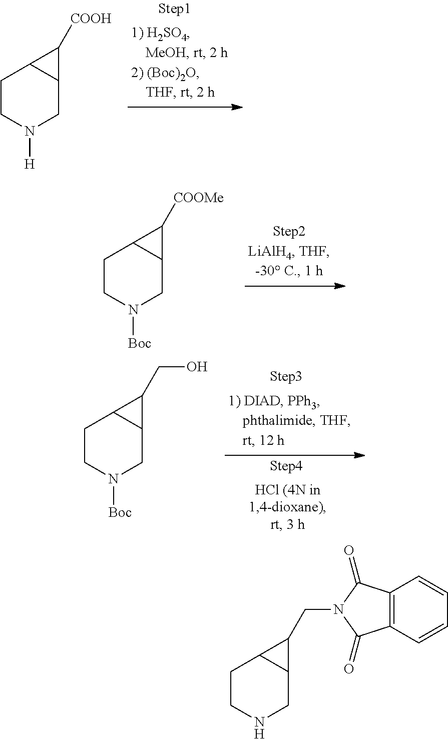

### Step 1: 3-(tert-butyl) 7-methyl 3-azabicyclo[4.1.0]heptane-3,7-dicarboxylate

To a solution of 3-azabicyclo[4.1.0]heptane-7-carboxylic acid hydrochloride (100 mg, 0.56 mmol) in MeOH (1 mL) conc. H₂SO₄ (1 μL) was added. The resulting mixture was stirred at rt for 2 h and then treated with 2M NaOH until pH=7.5. After dilution in THF (1 mL), (Boc)₂O (154 mg, 0.71 mmol) and DIPEA (0.19 mL, 1.06 mmol) were added. The resulting mixture was stirred for 2 h at rt; volatiles were evaporated, and the aqueous residue was extracted with EtOAc (×2). The collected organic layers were washed with brine (×2), dried over Na₂SO₄ filtered and concentrated in vacuo affording the title compound which was used in the next step without further purification (130 mg). LCMS (ES⁺) Method 1A: m/z 256 (M+H)⁺, RT 1.96 min.

### Step 2: tert-butyl 7-(hydroxymethyl)-3-azabicyclo[4.1.0]heptane-3-carboxylate

3-(Tert-butyl) 7-methyl 3-azabicyclo[4.1.0]heptane-3,7-dicarboxylate from previous step (130 mg, 0.51 mmol) was dissolved in dry THF (23 mL) under N₂ atmosphere, and the solution was cooled to −30° C. LiAlH₄ (solution 2M in THF, 0.28 mL, 0.56 mmol) was added dropwise, and the reaction was stirred between −30° C. and −20° C. for 1 h. The reaction was diluted with EtOAc (10 mL) and quenched with saturated solution of Rochelle salt and H₂O (10 mL). After about 30 min stirring at rt, the mixture was dried over Na₂SO₄, filtered, and concentrated under reduced pressure. The resulting crude compound was used in the next step without any further purification (100 mg). LCMS (ES⁺) Method 1A: m/z 228 (M+H)⁺, RT 1.50 min.

### Step 3: tert-butyl 7-((1,3-dioxoisoindolin-2-yl)methyl)-3-azabicyclo[4.1.0]heptane-3-carboxylate

Tert-butyl 7-(hydroxymethyl)-3-azabicyclo[4.1.0]heptane-3-carboxylate (100 mg, 0.44 mmol), phthalimide (77 mg, 0.53 mmol), PPh₃ (196 mg, 0.75 mmol) were dissolved in dry THF (3.5 mL) then DIAD (0.15 mL, 0.75 mmol) was added dropwise. The resulting yellow solution was stirred at rt for 2 h. Solvent was removed under reduced pressure and the resulting crude was purified by flash chromatography (10-20% EtOAc in petroleum ether), affording the title compound (100 mg, 64% over 3 steps). ¹H NMR (300 MHz, CDCl₃) δ 7.83-7.74 (m, 2H), 7.70-7.61 (m, 2H), 3.66-3.38 (m, 4H), 3.24-3.12 (m, 1H), 2.97-2.84 (m, 1H), 1.91-1.76 (m, 1H), 1.65-1.56 (m, 1H), 1.38-1.30 (m, 9H), 1.09-0.92 (m, 3H); LCMS (ES⁺) Method 1A: m/z 357 (M+H)⁺, RT 2.20 min.

### Step 4: 2-((3-azabicyclo[4.1.0]heptan-7-yl)methyl)isoindoline-1,3-dione

Tert-butyl 7-((1,3-dioxoisoindolin-2-yl)methyl)-3-azabicyclo[4.1.0]heptane-3-carboxylate (100 mg, 0.28 mmol) was treated with 1 mL of HCl (4N in 1,4-dioxane) and the resulting solution was stirred at rt for 3 h. After evaporation, the compound was used without further purification. LCMS (ES⁺) Method 1A: m/z 257 (M+H)⁺, RT 1.02 min.

### Intermediate 39: benzyl (((1S,6R,7S)-7-(4-methylthiazol-2-yl)-3-azabicyclo[4.1.0]heptan-7-yl)methyl)carbamate

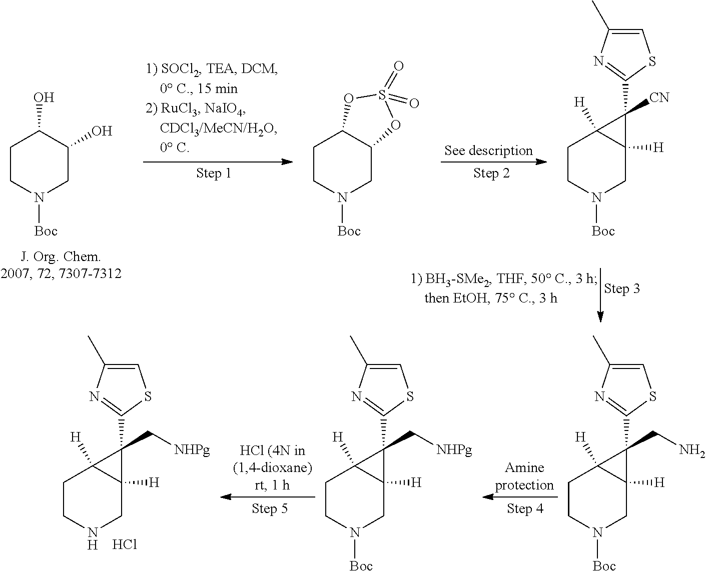

### Step 1: tert-butyl (3aR,7aS)-tetrahydro-[1,3,2]dioxathiolo[4,5-c]pyridine-5(4H)-carboxylate 2,2-dioxide

Step 1 was carried out according to the procedure described in the synthesis of Intermediate 1 step 2, starting from tert-butyl (3R,4S)-3,4-dihydroxypiperidine-1-carboxylate (prepared as described in J. Org. Chem 2007, 72, pp. 7307-7312; 170 mg, 0.78 mmol). LCMS (ES⁺) Method 1A: m/z 280 (M+H)⁺, RT 1.80 min.

### Step 2: tert-butyl (1S,6R,7S)-7-cyano-7-(4-methylthiazol-2-yl)-3-azabicyclo[4.1.0]heptane-3-carboxylate

Tert-butyl (3aR,7aS)-tetrahydro-[1,3,2]dioxathiolo[4,5-c]pyridine-5(4H)-carboxylate 2,2-dioxide (1000 mg, 3.58 mmol), 15-crown-5 (71 μL, 0.036 mmol) and 2-(4-methylthiazol-2-yl)acetonitrile (544 mg, 3.93 mmol) were dissolved in dry DME (7.2 mL) and the resulting solution was added dropwise over a period of 30 min to a suspension of NaH (60% in mineral oil; 372 mg, 9.3 mmol) in DME (5 mL) at rt under N₂ atmosphere. The reaction mixture was stirred at rt for 3 h, then quenched by addition of H₂O and extracted with EtOAc (×2). The combined organic layers were dried over Na₂SO₄, filtered and concentrated in vacuo. The residue was purified by flash chromatography (0-20% EtOAc in DCM) to give the title compound as a red oil (693 mg, 60%). ¹H NMR (300 MHz, CDCl₃) δ 6.76 (br d, J=0.9 Hz, 1H), 4.25 (d, J=4.8 Hz, 1H), 4.10-3.75 (m, 1H), 3.69 (dd, J=14.8, 5.0 Hz, 1H), 3.05-2.70 (m, 1H), 2.55-2.40 (m, 2H), 2.38 (d, J=0.9 Hz, 3H), 2.33-2.15 (m, 1H), 2.13-1.98 (m, 1H), 1.50 (s, 9H); LCMS (ES⁺) Method 1A: m/z 320 (M+H)⁺, RT 2.00 min.

### Step 3: tert-butyl (1S,6R,7S)-7-(aminomethyl)-7-(4-methylthiazol-2-yl)-3-azabicyclo[4.1.0]heptane-3-carboxylate

To a solution of tert-butyl (1S,6R,7S)-7-cyano-7-(4-methylthiazol-2-yl)-3-azabicyclo[4.1.0]heptane-3-carboxylate (680 mg, 2.13 mmol) in dry THF (0.16 M), BMS (2M in THF; 8.52 mmol) was added and the reaction mixture was stirred at 50° C. for 1 h. Additional BMS (2M in THF; 4.26 mmol) was added and the reaction mixture was stirred at 50° C. for 2 h. The reaction was quenched with EtOH (0.08M) and the resulting solution was stirred at 70° C. for 3 h. Volatiles were removed in vacuo and the residue was purified by flash chromatography (0-10% MeOH in DCM) to give the title compound as a pale yellow oil (280 mg, 41%). LCMS (ES⁺) Method 1A: m/z 324 (M+H)⁺, RT 1.29 min.

### Step 4: tert-butyl (1S,6R,7S)-7-((((benzyloxy)carbonyl)amino)methyl)-7-(4-methylthiazol-2-yl)-3-azabicyclo[4.1.0]heptane-3-carboxylate

The title compound was prepared according to the procedure described in the synthesis of Intermediate 28 step 3 starting from tert-butyl (1S,6R,7S)-7-(aminomethyl)-7-(4-methylthiazol-2-yl)-3-azabicyclo[4.1.0]heptane-3-carboxylate (91 mg, 0.28 mmol). LCMS (ES⁺) Method 1A: m/z 458 (M+H)⁺, RT 2.07 min.

### Step 5: benzyl (((1S,6R,7S)-7-(4-methylthiazol-2-yl)-3-azabicyclo[4.1.0]heptan-7-yl)methyl)carbamate

Tert-butyl (1S,6R,7S)-7-((((benzyloxy)carbonyl)amino)methyl)-7-(4-methylthiazol-2-yl)-3-azabicyclo[4.1.0]heptane-3-carboxylate (35 mg, 0.076 mmol) was treated with 1 mL of HCl (4N in 1,4-dioxane) and the resulting solution was stirred at rt for 3 h. After evaporation, the compound was used in the next step without further purification. LCMS (ES⁺) Method 1A: m/z 358 (M+H)⁺, RT 1.28 min.

### Intermediate 40: 2-(((1S,6R,7S)-7-(4-methylthiazol-2-yl)-3-azabicyclo[4.1.0]heptan-7-yl)methyl)isoindoline-1,3-dione

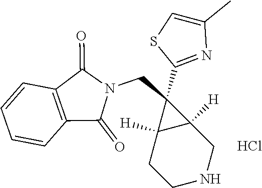 [SMILES: Cc1csc([C@@]2(CN3C(=O)c4ccccc4C3=O)[C@@H]3CCNC[C@@H]32)n1 ; Cl]

The compound was prepared following the procedure outlined for the synthesis of Intermediate 39, where in step 4 and step 5 were carried out as indicated below.

### Step 4: tert-butyl (1S,6R,7S)-7-((1,3-dioxoisoindolin-2-yl)methyl)-7-(4-methylthiazol-2-yl)-3-azabicyclo[4.1.0]heptane-3-carboxylate

The title compound was prepared according to the procedure described in Step 1 of the synthesis of Intermediate 36 starting from tert-butyl (1S,6R,7S)-7-(aminomethyl)-7-(4-methylthiazol-2-yl)-3-azabicyclo[4.1.0]heptane-3-carboxylate (198 mg, 0.61 mmol). LCMS (ES⁺) Method 1A: m/z 454 (M+H)⁺, RT 2.03 min.

### Step 5: 2-(((1S,6R,7S)-7-(4-methylthiazol-2-yl)-3-azabicyclo[4.1.0]heptan-7-yl)methyl)isoindoline-1,3-dione

Tert-butyl (1S,6R,7S)-7-((1,3-dioxoisoindolin-2-yl)methyl)-7-(4-methylthiazol-2-yl)-3-azabicyclo[4.1.0]heptane-3-carboxylate (194, 0.43 mmol) was treated with 1 mL of HCl (4N in 1,4-dioxane) and the resulting solution was stirred at rt for 3 h. After evaporation, the compound was used without further purification. LCMS (ES⁺) Method 1A: m/z 354 (M+H)⁺, RT 1.09 min.

### Intermediate 41: 6-chloro-3-((2-(trifluoromethyl)pyridin-3-yl)thio)pyrazin-2-amine

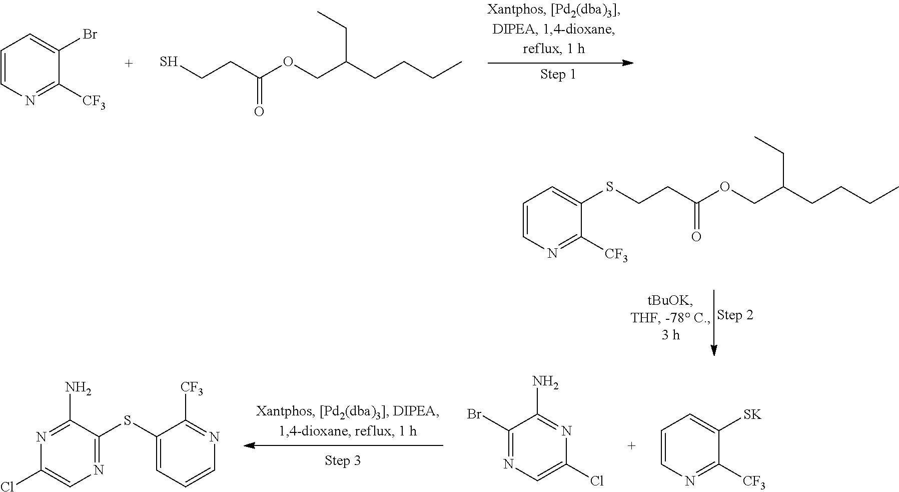

### Step 1: 2-ethylhexyl 3-((2-(trifluoromethyl)pyridin-3-yl)thio)propanoate

To a mixture of 3-bromo-2-trifluoromethylpyridine (712 mg, 3.150 mmol), 2-ethylhexyl 3-sulfanylpropanoate (0.788 mL, 3.465 mmol), Pd₂(dba)₃ (289 mg, 0.316 mmol), XantPhos (183 mg, 0.316 mmol) in dry degassed 1,4 dioxane (7 mL) under N₂ atmosphere, DIPEA (1.7 mL, 9.76 mmol) was added, and the resulting mixture was heated at 100° C. for 1 h. After solvent concentration, the residue was purified by flash chromatography (0-20% EtOAc in cyclohexane) to afford the title compound as an orange solid (809 mg, 70%). ¹H NMR (300 MHz, DMSO-d₆) δ 8.52 (br d, J=4.2 Hz, 1H), 8.17 (br d, J=8.3 Hz, 1H), 7.72-7.65 (m, 1H), 3.95 (d, J=5.5 Hz, 2H), 3.42-3.33 (m, 2H), 2.69 (t, J=6.5 Hz, 2H), 1.34-1.18 (m, 9H), 0.89-0.79 (m, 6H). LCMS (ES⁺) Method 3: m/z 364 (M+H)⁺, RT 2.08 min.

### Step 2: potassium 2-(trifluoromethyl)pyridine-3-thiolate

To a solution of 2-ethylhexyl 3-((2-(trifluoromethyl)pyridin-3-yl)thio)propanoate (500 mg, 1.38 mmol) in dry THF (4 mL) cooled to −78° C., a suspension of tBuOK (463.1 mg, 4.13 mmol) in dry THF (20 mL) was added. After stirring at −78° C. for 20 min the reaction was quenched with aqueous K₂CO₃ (2M, 0.25 mL) and gradually warmed to rt (a brown solid precipitated). The solid was filtered off, washed with THF (×2) and dried under reduced pressure, to afford the title compound as a brownish solid (300 mg, 98%). ¹H NMR (300 MHz, DMSO-d₆) δ 7.63 (d, J=4.0 Hz, 1H), 7.52 (d, J=8.3 Hz, 1H), 6.77 (dd, J=8.1, 4.2 Hz, 1H). LCMS (ES⁺) Method 1A: m/z 180 (M+H)⁺, RT 1.69 min.

### Step 3: 6-chloro-3-((2-(trifluoromethyl)pyridin-3-yl)thio)pyrazin-2-amine

To a solution of 2-amino-3-bromo-6-chloropyrazine (464 mg, 2.2261 mmol) and potassium 2-(trifluoromethyl)pyridine-3-thiolate (483.6 mg, 2.23 mmol) in degassed dry 1,4-dioxane (10 mL) in a sealed tube, Pd₂(dba)₃ (204 mg, 0.22 mmol), XantPhos (129 mg, 0.22 mmol) were sequentially added followed by dry DIPEA (1 mL, 5.74 mmol) under N₂ atmosphere. The resulting mixture was refluxed for 1 h. The volatiles were evaporated under reduced pressure and the resulting residue purified by flash chromatography (0-30% EtOAc in cyclohexane) to afford the title compound as yellow solid (390 mg, 57%). ¹H NMR (300 MHz, DMSO-d₆) δ 8.71 (dd, J=4.5, 1.0 Hz, 1H), 8.00-7.94 (m, 1H), 7.74-7.68 (m, 2H), 7.16 (s, 2H). LCMS (ES⁺) Method 1A: m/z 307, 309 (M+H)⁺, RT 1.96 min.

### Intermediate 42: 3-((2-amino-3-chloropyridin-4-yl)thio)-6-chloropyrazin-2-amine

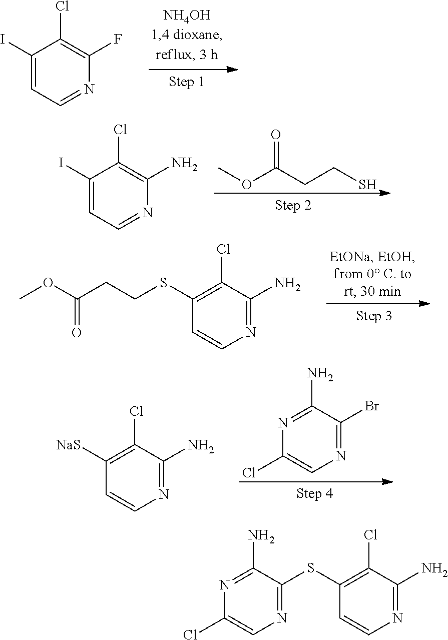

### Step 1: 3-chloro-4-iodopyridin-2-amine

A solution of 3-chloro-2-fluoro-4-iodopyridine (2.0 g, 7.769 mmol) in 1,4 dioxane (8 mL) and 30% NH₄₀H (20 mL; 155.3 mmol) in H₂O was refluxed for 3 h. The mixture was diluted with EtOAc and H₂O, the phases were separated and the aqueous layer was extracted with EtOAc (3×10 mL). The collected organic layers were washed with brine (×2), dried over Na₂SO₄, filtered and concentrated in vacuo to afford the title compound as a brownish solid (1.96 g, quantitative). ¹H NMR (300 MHz, DMSO-d₆) δ 7.59 (d, J=5.14 Hz, 1H), 7.11 (d, J=5.14 Hz, 1H), 6.56 (s, 2H). LCMS (ES⁺) Method 1A: m/z 255 (M+H)⁺, RT 0.92 min.

### Step 2: methyl 3-((2-amino-3-chloropyridin-4-yl)thio)propanoate

To a solution of 3-chloro-4-iodopyridin-2-amine (1.96 g, 7.70 mmol) and methyl 3-mercaptopropionate (0.92 mL, 8.48 mmol) in degassed dry 1,4-dioxane (30 mL), Pd₂(dba)₃ (1 g, 1.15 mmol), [(t-Bu)₃PH]BF₄ (334 mg, 1.15 mmol) were sequentially added followed by degassed dry DIPEA (4.0 mL, 23.76 mmol) under N₂ atmosphere. The mixture was stirred at 90° C. for 2 h. The volatiles were removed under reduced pressure and the residue was purified by flash chromatography (20-100% EtOAc in petroleum ether) to afford methyl 3-((2-amino-3-chloropyridin-4-yl)thio)propanoate as an orange solid (1.8 g, 93%). ¹H NMR (300 MHz, DMSO-d₆) δ 7.81 (d, J=5.3 Hz, 1H), 6.57 (d, J=5.4 Hz, 1H), 6.25 (s, 2H) 3.64 (s, 3H), 3.22 (t, J=7.0 Hz, 2H), 2.73 (t, J=7.0 Hz, 2H); LCMS (ES⁺) Method 1A: m/z 247 (M+H)⁺, RT 0.98 min.

### Step 3: 2-amino-3-bromo-6-chloropyrazine

To a solution of methyl 3-((2-amino-3-chloropyridin-4-yl)thio)propanoate(1.8 g, 7.29 mmol) in EtOH (22 mL), EtONa (solution 20% wt in EtOH, 3.6 mL, 9.24 mmol) was added at 0° C. The mixture was gradually warmed to rt. After 30 min EtOH was removed under reduced pressure and the residue diluted with DCM (50 mL). Product precipitation occurred, and the solid was filtered and washed with DCM (×3) to afford the title compound as a red solid (1.3 g, 92%). ¹H NMR (300 MHz, DMSO-d₆) δ 7.03 (d, J=5.4 Hz, 1H), 6.46 (d, J=5.3 Hz, 1H), 5.01 (s, 2H). LCMS (ES⁺) Method 1A: m/z 161,163 (M+H)⁺, RT 0.56 min.

### Step 4: 3-((2-amino-3-chloropyridin-4-yl)thio)-6-chloropyrazin-2-amine

To a solution of 2-amino-3-bromo-6-chloropyrazine (419 mg, 2.01 mmol) and sodium 2-amino-3-chloropyridine-4-thiolate (367 mg, 2.01 mmol) in degassed dry 1,4-dioxane (8 mL), Pd₂(dba)₃ (184 mg, 0.20 mmol) and [(t-Bu)₃PH]BF₄ (58.5 mg, 0.202 mmol) were sequentially added followed by dry DIPEA (0.87 mL, 5.02 mmol) under N₂ atmosphere. The resulting mixture was stirred at 105° C. for 1.5 h. Volatiles were removed under reduced pressure and the residue was purified by flash chromatography (C18; 5-45% MeCN/H₂O+0.1% HCOOH) to afford the title compound as an orange solid (116 mg, 20%). ¹H NMR (300 MHz, DMSO-d₆) δ 7.89 (s, 1H), 7.70 (d, J=5.3 Hz, 1H), 7.17 (s, 2H), 6.39 (s, 2H), 5.97 (d, J=5.3 Hz, 1H). LCMS (ES⁺) Method 1A: m/z 288 (M+H)⁺, RT 1.13 min

### Intermediate 43: 8-bromo-5-chloroimidazo[1,2-c]pyrimidine

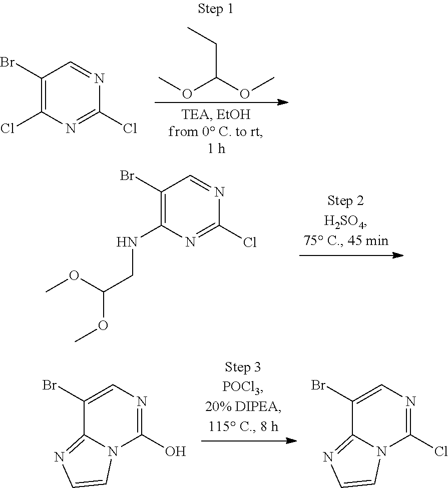

### Step 1: 5-bromo-2-chloro-N-(2,2-dimethoxyethyl)pyrimidin-4-amine

Aminoacetaldehyde dimethyl acetal (1.5 mL, 13.76 mmol) was added to a solution of 5-bromo-2,4-dichloro-pyrimidine (2.05 g, 8.99 mmol) and dry TEA (2.0 mL, 14.35 mmol) in EtOH (15 mL) at 0° C. The mixture was gradually warmed to rt and stirred for 1 h. The mixture was concentrated, then diluted with EtOAc and washed with H₂O (×2). The organic layer was washed with brine (×2) dried over Na₂SO₄, filtered, and concentrated under reduced pressure to afford the title compound as a white powder (2.63 g, 98%). ¹H NMR (300 MHz, DMSO-d₆) δ 8.28 (s, 1H), 7.65 (br t, J=5.7 Hz, 1H), 4.58 (t, J=5.4 Hz, 1H), 3.48 (t, J=5.7 Hz, 2H), 3.31-3.26 (m, 6H); LCMS (ES⁺) Method 1A: m/z 296 (M+H)⁺, RT 1.62 min.

### Step 2: 8-bromoimidazo[1,2-c]pyrimidin-5-ol

A suspension of 5-bromo-2-chloro-N-(2,2-dimethoxyethyl)pyrimidin-4-amine (2.63 g, 8.86 mmol) in conc. H₂SO₄ (8 mL) was stirred at 75° C. for 40 min. The mixture was cooled at 0° C. and 5N NaOH (50 mL) was added until pH=6 and a precipitate was observed. The solid was filtered, and the filtrate was extracted with EtOAc (3×5 mL). The collected organic layers were washed with brine (×2) dried over Na₂SO₄, filtered, and concentrated in vacuo to afford a beige powder (1.52 g). ¹H NMR (300 MHz, DMSO-d₆) δ 11.90 (br s, 1H), 7.89 (d, J=1.5 Hz, 1H), 7.64 (d, J=4.6 Hz, 1H), 7.43 (d, J=1.6 Hz, 1H); LCMS (ES⁺) Method 1A: m/z 214 (M+H)⁺, RT 0.64 min.

### Step 3: 8-bromo-5-chloroimidazo[1,2-c]pyrimidine

In a sealed microwave vial, dry DIPEA (1.6 mL) was added to a suspension of 8-bromoimidazo[1,2-c]pyrimidin-5-ol (380 mg, 1.78 mmol) in POCl₃ (8 mL) at 0° C. The mixture was stirred at 115° C. for 9 h and then cooled and slowly added to a stirred mixture of cold H₂O and EtOAc. The organic layer was washed with H₂O (2×10 mL), dried over Na₂SO₄, filtered and concentrated under reduced pressure. The crude compound was purified by flash chromatography (C18; 0-35% MeCN/H₂O+0.1% TFA) to afford the title compound as beige powder (170 mg, 41%). ¹H NMR (300 MHz, DMSO-d₆) δ 8.22-8.18 (m, 2H) 7.85 (d, J=1.5 Hz, 1H). LCMS (ES⁺) Method 1A: m/z 232 (M+H)⁺, RT 1.21 min.

### Intermediate 44: 6-chloro-3-iodo-1-(tetrahydro-2H-pyran-2-yl)-1H-pyrazolo[3,4-b]pyrazine

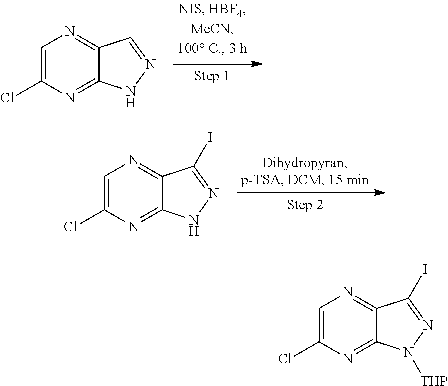

### Step 1: 6-chloro-3-iodo-1H-pyrazolo[3,4-b]pyrazine

6-Chloro-1H-pyrazolo[3,4-b]pyrazine (850 mg, 5.5 mmol) was suspended in MeCN (7 mL) then NIS (1.86 g, 8.25 mmol) and HBF₄ (48% in H₂O 1.72 mL, 13.14 mmol) were sequentially added. The resulting mixture was refluxed for 3 h, then cooled by ice bath and filtered. The yellow precipitate was washed with cold MeCN (×3) to obtain the title compound as a yellow solid (1.25 g, 81%). ¹H NMR (300 MHz, DMSO-d₆) δ 14.66 (br s, 1H), 8.70 (s, 1H); LCMS (ES⁺) Method 1A: m/z 281 (M+H)⁺, RT 1.65 min.

### Step 2: 6-chloro-3-iodo-1-(tetrahydro-2H-pyran-2-yl)-1H-pyrazolo[3,4-b]pyrazine

6-Chloro-3-iodo-1H-pyrazolo[3,4-b]pyrazine (754 mg, 2.69 mmol) was dissolved in DCM (13 mL) and treated with DHP (0.76 mL, 8.33 mmol) and PTSA (155 mg, 0.90 mmol). The reaction was stirred at rt for 30 min, then was poured into NaHCO₃ sat. sol. (30 mL) and extracted with DCM (×3). The combined organic extracts were evaporated in vacuo and the residue was purified by flash chromatography (0-20% EtOAc in petroleum ether) to afford the title compound as a light-yellow solid (600 mg, 61%). ¹H NMR (300 MHz, CDCl₃) δ 8.59 (s, 1H), 5.99 (dd, J=10.4, 2.6 Hz, 1H), 4.14 (m, 1H), 3.82 (m, 1H), 2.67 (m, 1H), 2.19 (m, 1H), 2.00 (m, 1H), 1.92-1.74 (m, 2H), 1.67 (m, 1H); LCMS (ES⁺) Method 1A: m/z 365 (M+H)⁺, RT 2.16 min.

### Intermediate 45: 4-chloro-2-methyl-5-(4,4,5,5-tetramethyl-1,3,2-dioxaborolan-2-yl)-2H-indazole

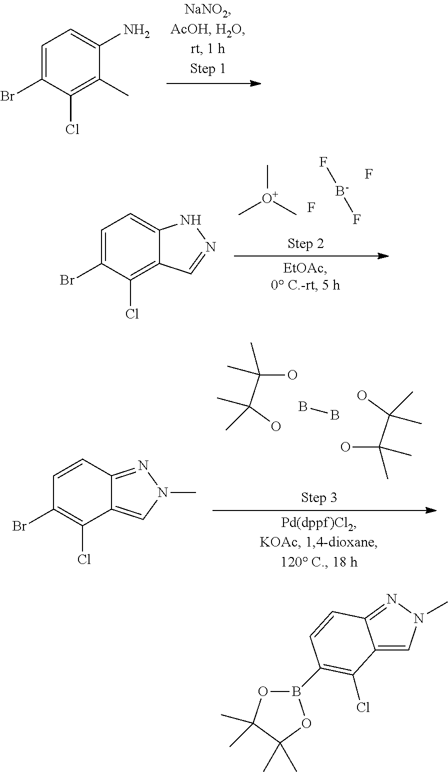

### Step 1: 5-bromo-4-chloro-1H-indazole

A solution of 4-bromo-3-chloro-2-methylaniline (867 mg, 3.93 mmol) in AcOH (17 mL) was treated with NaNO₂ (339.0 mg, 4.91 mmol) in H₂O (1.5 mL). The mixture was stirred at rt for 1 h then concentrated and the residue was suspended in H₂O, filtered, and washed with H₂O and n-heptane. The organic phase was concentrated in vacuo to give a residue which was purified by flash chromatography (0-30% EtOAc in cyclohexane) to afford the title compound as a light orange solid (328 mg, 36%). LCMS (ES⁺) Method 2: m/z 231 (M+H)⁺, RT 1.59 min.

### Step 2: 5-bromo-4-chloro-2-methyl-2H-indazole

A solution of 5-bromo-4-chloro-1H-indazole (1.49 g, 6.45 mmol) in EtOAc (32 mL) at 0° C. was treated with trimethyloxonium tetrafluoroborate (1.43 g, 9.65 mmol). The resultant mixture was allowed to warm to rt and stirred for 5 h. After completion, the mixture was quenched with NaHCO₃ sat. sol. and extracted with EtOAc. The organic layer was washed with brine, dried over Na₂SO₄, filtered, and concentrated in vacuo. The crude product was purified by flash chromatography (0-30% EtOAc in n-heptane) to afford the title compound as an orange solid (900 mg, 57%). ¹H NMR (500 MHz, DMSO-d₆): δ 8.49 (s, 1H), 7.52 (d, J=9.0 Hz, 1H), 7.44 (d, J=9.0 Hz, 1H), 4.16 (s, 3H). LCMS (ES⁺) Method 2: m/z 245 (M+H)⁺, RT 1.69 min.

### Step 3: 4-chloro-2-methyl-5-(4,4,5,5-tetramethyl-1,3,2-dioxaborolan-2-yl)-2H-indazole

A suspension of 5-bromo-4-chloro-2-methyl-2H-indazole (548.0 mg, 2.23 mmol), B₂pin₂ (1.36 g, 5.36 mmol), Pd(dppf)Cl₂·DCM (163.3 mg, 0.223 mmol) and KOAc (657.2 mg, 6.70 mmol) in 1,4-dioxane (11 mL) was heated to 120° C. for 18 h. After completion, the mixture was cooled to rt, the solid precipitate was filtered off and washed with EtOAc. The filtrate was concentrated in vacuo and the residue was dissolved in toluene and n-heptane until precipitation occurred. The suspension was filtered through a pad of Celite and the filtrate was concentrated in vacuo. The residue was triturated with n-heptane, the solid was filtered off and the filtrate was concentrated under reduced pressure to afford the title compound (1.15 g, 99%) as a light brown solid. ¹H NMR (300 MHz DMSO-d₆) δ 8.52 (s, 1H), 7.50 (d, J=8.7 Hz, 1H), 7.42 (d, J=8.7 Hz, 1H), 4.18 (s, 3H), 1.32 (s, 12H). LCMS (ES⁺) Method 2: m/z 293 (M+H)⁺, RT 1.89 min.

### Intermediate 46: 3,4-dichloro-2-methyl-5-(4,4,5,5-tetramethyl-1,3,2-dioxaborolan-2-yl)-2H-indazole

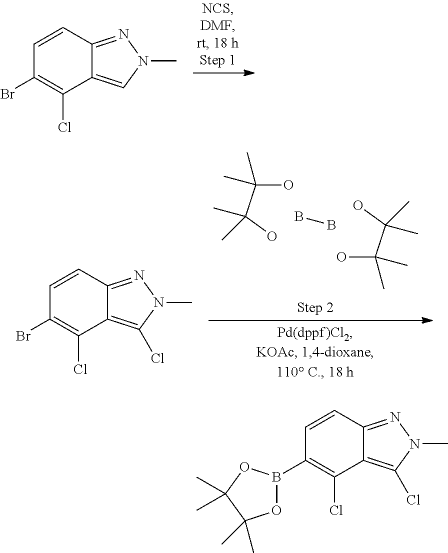

### Step 1: 5-bromo-3,4-dichloro-2-methyl-2H-indazole

5-Bromo-4-chloro-2-methyl-2H-indazole (1.0 g, 4.1 mmol) in dry DMF (20 mL) was treated with NCS (575.7 mg, 4.3 mmol). The mixture was stirred at rt for 18 h. After completion, the mixture was diluted with H₂O and the precipitated solid was filtered off, washed with H₂O and dried. The crude product was triturated with a mixture of EtOAc and n-heptane, then washed with n-heptane and dried to afford the title compound (863 mg, 75%) as a yellow solid. LCMS (ES⁺) m/z 279 (M+H)+

### Step 2: 3,4-dichloro-2-methyl-5-(4,4,5,5-tetramethyl-1,3,2-dioxaborolan-2-yl)-2H-indazole

A suspension of 5-bromo-3,4-dichloro-2-methyl-2H-indazole (647.0 mg, 2.31 mmol), B₂pin₂ (1.17 g, 4.62 mmol), Pd(dppf)Cl₂·DCM (169.1 mg, 0.23 mmol) and KOAc (680.5 mg, 6.93 mmol) in 1,4-dioxane (11.5 mL) was heated at 110° C. for 18 h in a pressure vessel. After completion, the mixture was cooled to rt, the solid precipitate was filtered off and washed with EtOAc. The filtrate was concentrated in vacuo and n-heptane was added. The precipitate was filtered off and the filtrate was concentrated under reduced pressure. The crude product was triturated with MeCN and dried to afford the title compound (168 mg, 99%). ¹H NMR (500 MHz, DMSO-d₆) δ 7.50 (d, J=8.8 Hz, 1H), 7.40 (d, J=8.8 Hz, 1H), 4.10 (s, 3H), 1.29 (s, 12H). LCMS (ES⁺): m/z 327 (M+H)⁺

### Intermediate 47: 6-chloro-3-iodo-5-methyl-1-(tetrahydro-2H-pyran-2-yl)-1,5-dihydro-4H-pyrazolo[3,4-d]pyrimidin-4-one

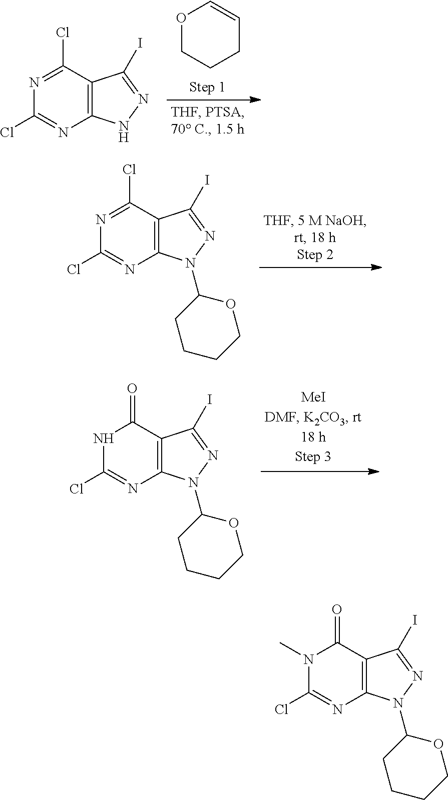

### Step 1: 4,6-dichloro-3-iodo-1-(tetrahydro-2H-pyran-2-yl)-1H-pyrazolo[3,4-d]pyrimidine

A solution of 4,6-dichloro-3-iodo-1H-pyrazolo[3,4-d]pyrimidine (2.0 g, 6.35 mmol), DHP (1.68 mL, 18.4 mmol) and PTSA (241.6 mg, 1.27 mmol) in THF (29 mL) was heated at 70° C. for 1.5 h. After completion, the solvent was removed in vacuo and the crude product was purified by flash chromatography (0-5% MeOH in CHCl₃) to afford the title compound as white solid (2.23 g, 88%). LCMS (ES⁺): m/z 399 (M+H)⁺, RT 2.20 min.

### Step 2: 6-chloro-3-iodo-1-(tetrahydro-2H-pyran-2-yl)-1,5-dihydro-4H-pyrazolo[3,4-d]pyrimidin-4-one

A solution of 4,6-dichloro-3-iodo-1-(tetrahydro-2H-pyran-2-yl)-1H-pyrazolo[3,4-d]pyrimidine (2.82 g, 7.0 mmol) in THF (28 mL) was treated with 5 M aq. NaOH solution (12.7 mL, 63.45 mmol). The mixture was stirred at rt for 5 h, then THF (14 mL) was added. The resultant mixture was stirred at rt for 18 h and then was acidified with 6 M aqueous HCl solution and extracted with EtOAc. The organic layer was washed with brine, dried over Na₂SO₄, filtered, and concentrated in vacuo. The crude product was triturated with n-heptane and dried to afford the title compound (1.8 g, 67%) as a white solid. ¹H NMR (300 MHz, DMSO-d₆) δ 13.40 (s, 1H), 5.66 (dd, J=10.2, 2.2 Hz, 1H), 3.91 (d, J=11.3 Hz, 1H), 3.71-3.59 (m, 1H), 2.35-2.17 (m, 1H), 2.04-1.90 (m, 1H), 1.89-1.78 (m, 1H), 1.77-1.62 (m, 1H), 1.60-1.48 (m, 2H).

### Step 3: 6-chloro-3-iodo-5-methyl-1-(tetrahydro-2H-pyran-2-yl)-1,5-dihydro-4H-pyrazolo[3,4-d]pyrimidin-4-one

A solution of 6-chloro-3-iodo-1-(tetrahydro-2H-pyran-2-yl)-1,5-dihydro-4H-pyrazolo[3,4-d]pyrimidin-4-one (1.75 g, 4.6 mmol) in DMF (21 mL) was treated with K₂CO₃ (762.6 mg, 5.5 mmol) followed by slow addition of Mel (314.9 μL, 5.0 mmol). The mixture was stirred at rt for 18 h. After completion, the mixture was diluted with H₂O and the solid precipitate was collected by filtration. The crude product was triturated with EtOAc to afford the title compound (1.04 g, 57%) as a white solid. ¹H NMR (500 MHz, CDCl₃) δ 5.73 (dd, J=10.2, 1.5 Hz, 1H), 4.08 (d, J=11.5 Hz, 1H), 3.73 (t, J=11.3 Hz, 1H), 3.68 (s, 3H), 2.54-1.43 (m, 1H), 2.09 (d, J=10.7 Hz, 1H), 1.86 (d, J=13.3 Hz, 1H), 1.82-1.65 (m, 2H), 1.58 (d, J=12.3 Hz, 1H). LCMS (ES⁺) Method 2: m/z 311 (M+H-THP)⁺, RT 1.76 min.

### Intermediate 48: 3-chloro-4-((6-chloro-5H-pyrrolo[2,3-b]pyrazin-2-yl)thio)pyridin-2-amine

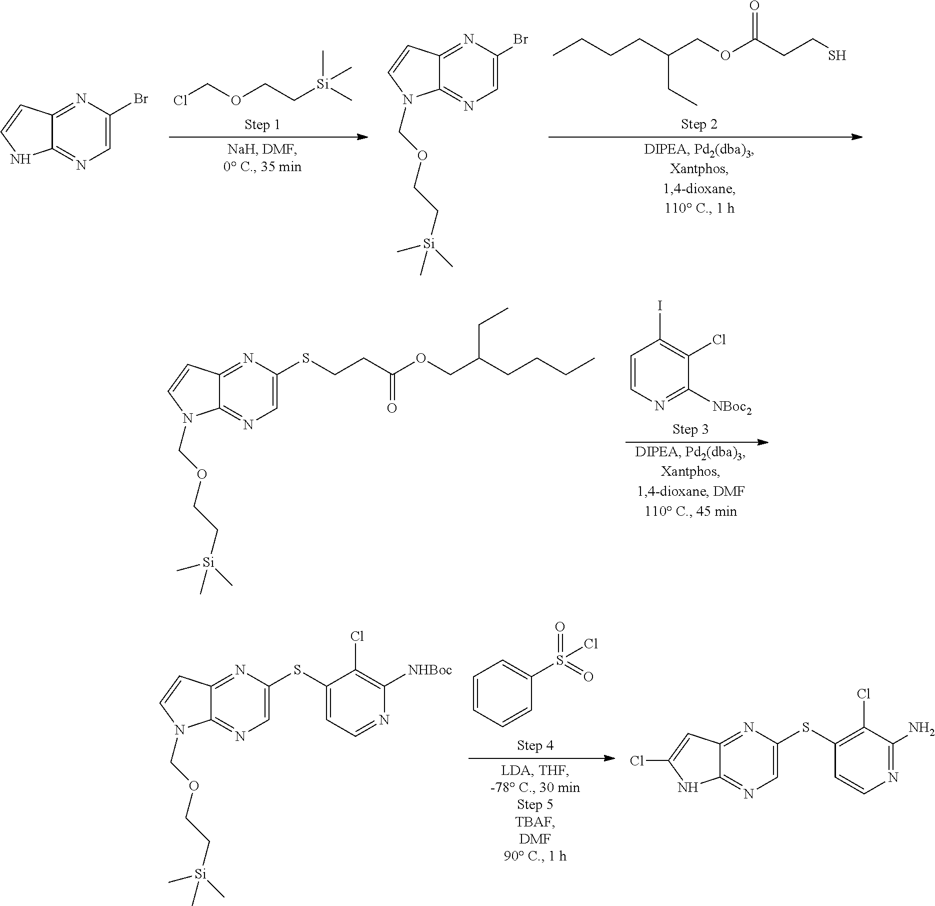

### Step 1: 2-bromo-5-((2-(trimethylsilyl)ethoxy)methyl)-5H-pyrrolo[2,3-b]pyrazine

2-Bromo-4H-pyrrolo[2,3-b]pyrazine (1 g, 5.05 mmol) was dissolved in DMF (34 mL) and the resulting solution was cooled to 0° C. and NaH (60% in mineral oil; 242 mg, 6.06 mmol) was added. The mixture was stirred at this temperature for 30 min then 2-(chloromethoxy)ethyl-trimethyl-silane (0.99 mL, 5.55 mmol) was added and the mixture stirred for 5 min. NaHCO₃ sat. sol. and EtOAc were added to the mixture. The organic phase was then isolated, washed with H₂O, dried over Na₂SO₄, filtered, and concentrated in vacuo to give a residue which was purified by flash chromatography (0-60% EtOAc in petroleum ether). The title compound was obtained as a pale-yellow oil (1410 mg, 85%). ¹H NMR (400 MHz, DMSO-d₆) δ 8.45 (s, 1H), 8.14 (d, J=4.0 Hz, 1H), 6.73 (d, J=4.0 Hz, 1H), 5.63 (s, 2H), 3.51 (t, J=8.0 Hz, 2H), 0.81 (t, J=8.0 Hz, 2H), 0.11 (s, 9H). LCMS (ES⁺) Method 2: m/z 328-330 (M+H)⁺, RT 2.36 min.

### Step 2: 2-ethylhexyl 3-((5-((2-(trimethylsilyl)ethoxy)methyl)-5H-pyrrolo[2,3-b]pyrazin-2-yl)thio)propanoate

2-Ethylhexyl 3-sulfanylpropanoate (1032 mg, 4.72 mmol), DIPEA (1665 mg, 12.89 mmol), Pd₂(dba)₃ (393 mg, 0.430 mmol) and XantPhos (249 mg, 0.430 mmol) were dissolved in 1,4-dioxane (11 mL) and 2-bromo-5-((2-(trimethylsilyl)ethoxy)methyl)-5H-pyrrolo[2,3-b]pyrazine (1410 mg, 4.3 mmol) was added. The mixture was stirred at 110° C. for 1 h then the solvent was concentrated in vacuo and the residue purified by flash chromatography (0-100% EtOAc in petroleum ether). The title compound was obtained as a yellow oil (2 g, 66% pure, 66%). LCMS (ES⁺) Method 2: m/z 466 (M+H)⁺, RT 2.55 min.

### Step 3: tert-butyl (3-chloro-4-((5-((2-(trimethylsilyl)ethoxy)methyl)-5H-pyrrolo[2,3-b]pyrazin-2-yl)thio)pyridin-2-yl)carbamate

A suspension of 2-ethylhexyl 3-((5-((2-(trimethylsilyl)ethoxy)methyl)-5H-pyrrolo[2,3-b]pyrazin-2-yl)thio)propanoate (1.790 g, 3.84 mmol), tert-butyl (tert-butoxycarbonyl)(3-chloro-4-iodopyridin-2-yl)carbamate (1.850 g, 4.07 mmol), Pd₂(dba)₃ (281.57 mg, 0.310 mmol) and XantPhos (222.39 mg, 0.380 mmol) in a mixture of 1,4-dioxane (11.7 mL) and DMF (3.7 mL) was degassed with N₂ then treated with tBuOK (8.46 mL, 8.46 mmol) and DIPEA (1.33 mL, 7.69 mmol). The reaction mixture was stirred for 45 min at 110° C. After cooling the solvent was concentrated under reduced pressure then DCM was added, and the precipitated powder was filtered. The organic solvent was concentrated in vacuo and the crude product was purified by flash chromatography (0-100% EtOAc in petroleum ether) to afford the title compound as brown solid (1.35 g, 69%). LCMS (ES⁺) Method 2: m/z 506 (M−H)⁻, RT 2.47 min.

### Step 4: tert-butyl (3-chloro-4-((6-chloro-5-((2-(trimethylsilyl)ethoxy)methyl)-5H-pyrrolo[2,3-b]pyrazin-2-yl)thio)pyridin-2-yl)carbamate

A solution of tert-butyl (3-chloro-4-((5-((2-(trimethylsilyl)ethoxy)methyl)-5H-pyrrolo[2,3-b]pyrazin-2-yl)thio)pyridin-2-yl)carbamate (600 mg, 1.18 mmol) in THF (7.4 mL) cooled to −78° C. was treated with LDA (1.18 mL, 1.18 mmol) and the mixture was stirred at this temperature for 30 min then benzenesulfonyl chloride (0.18 mL, 1.42 mmol) was added and the reaction mixture stirred for 5 min. NaHCO₃ sat. sol. and EtOAc were added then the organic phase was separated and washed with Na₂S₂O₃ sat sol, dried over Na₂SO₄, filtered and concentrated in vacuo. The crude product was purified by flash chromatography (0-100% EtOAc in petroleum ether) to afford the title compound as a yellow solid (300 mg, 47%). LCMS (ES⁺) Method 2: m/z 540 (M−H)⁻, RT 2.6 min.

### Step 5: 3-chloro-4-((6-chloro-5H-pyrrolo[2,3-b]pyrazin-2-yl)thio)pyridin-2-amine

A solution of tert-butyl (3-chloro-4-((6-chloro-5-((2-(trimethylsilyl)ethoxy)methyl)-5H-pyrrolo[2,3-b]pyrazin-2-yl)thio)pyridin-2-yl)carbamate (50 mg, 0.090 mmol) in DMF (0.74 mL) was treated with TBAF (0.28 mL, 0.280 mmol) and the mixture was stirred for 1 h at 90° C. After cooling the solvent was removed in vacuo and the crude product was purified by flash chromatography (0-100% EtOAc in petroleum ether) to afford the title compound as yellow solid (16.7 mg, 58%). LCMS (ES⁺) Method 2: m/z 311 (M+H)⁺, RT 0.77 min.

### Intermediate 49: 2-chloro-6-((2-(trifluoromethyl)pyridin-3-yl)thio)-1H-imidazo[4,5-b]pyrazine

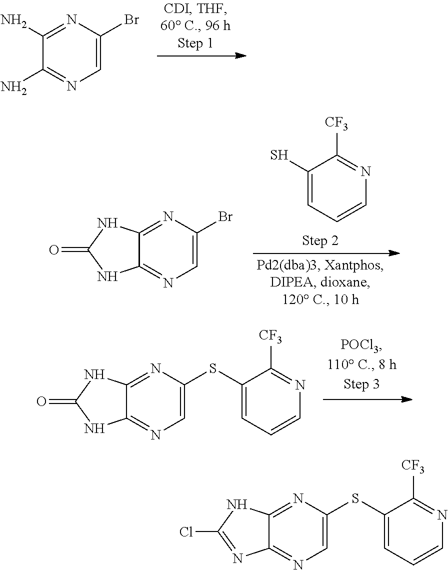 [SMILES: Nc1ncc(Br)nc1N ; O=c1[nH]c2ncc(Br)nc2[nH]1 ; FC(F)(F)c1ncccc1S ; O=c1[nH]c2ncc(Sc3cccnc3C(F)(F)F)nc2[nH]1 ; FC(F)(F)c1ncccc1Sc1cnc2nc(Cl)[nH]c2n1]

### Step 1: 5-bromo-1,3-dihydro-2H-imidazo[4,5-b]pyrazin-2-one

A solution of 5-bromo-2,3-pyrazindiamine (4.1 g, 21.7 mmol) and CDI (7.0 g, 43.4 mmol) in THF (120 mL) was heated to 60° C. for 72 h. Then, a second portion of CDI (7.0 g, 43.4 mmol) was added and heating to 60° C. was continued for 24 h. After cooling, the mixture was concentrated in vacuo and the residue was purified by flash chromatography (C18; 0-50% MeCN/H₂O+0.1% TFA). Evaporation of the fractions containing the desired product furnished a compound which was triturated with MeCN and filtered off to give the title compound as a beige solid (3.04 g, 65%). ¹H NMR (300 MHz, DMSO-d₆) δ 11.98 (br s, 1H), 11.91 (br s, 1H), 7.98 (s, 1H). LCMS (ES⁺) Method 2: m/z 215, 217 (M+H)⁺, RT 0.76 min.

### Step 2: 5-[(2-(trifluoromethyl)pyridin-3-yl)thio]-1,3-dihydro-2H-imidazo[4,5-b]pyrazin-2-one

A solution of 5-bromanyl-1,3-dihydroimidazo[4,5-b]pyrazin-2-one (1.0 g, 4.65 mmol) in 1,4-dioxane (47 mL) was degassed with N₂ for 5 min, then Pd₂(dba)₃ (0.21 g, 0.23 mmol), XantPhos (0.27 g, 0.47 mmol), 2-(trifluoromethyl)pyridine-3-thiol (1.19 g, 5.12 mmol) and DIPEA (1.62 mL, 9.3 mmol) were sequentially added. The reaction mixture was degassed with N₂ for further 5 min and heated to 100° C. for 2 h. After cooling, the mixture was concentrated in vacuo and the residue was treated with MeCN affording a precipitate which was filtered off to give the title compound as a beige solid (1.33 g, 91%). ¹H NMR (300 MHz, DMSO-d₆) δ 11.96 (br s, 2H), 8.56 (d, J=3.7 Hz, 1H), 8.10 (s, 1H), 7.75 (d, J=8.3 Hz, 1H), 7.58 (dd, J=8.2, 4.5 Hz, 1H). LCMS (ES⁺) Method 2: m/z 314 (M+H)⁺, RT 1.24 min.

### Step 3: 2-chloro-6-[(2-(trifluoromethyl)pyridin-3-yl)thio]-1H-imidazo[4,5-b]pyrazine

A solution of 5-[2-(trifluoromethyl)pyridin-3-yl]sulfanyl-1,3-dihydroimidazo[4,5-b]pyrazin-2-one (1.3 g, 4.15 mmol) in POCl₃ (20 mL, 0.21 mol) was heated to 120° C. in a sealed vial for 8 h. After cooling, the mixture was concentrated in vacuo to give a residue which was purified by flash chromatography (C18; 0-100% MeCN/H₂O+0.1% TFA) to furnish the title compound as a pale-yellow solid (TFA salt; 0.26 g, 19%). ¹H NMR (300 MHz, DMSO-d₆) δ 8.71 (d, J=3.9 Hz, 1H), 8.55 (s, 1H), 8.05 (d, J=7.9 Hz, 1H), 7.70 (dd, J=8.1, 4.6 Hz, 1H). LCMS (ES⁺) Method 2: m/z 332 (M+H)⁺, RT 1.4 min.

### Intermediate 50: 3-chloro-4-((2-(methylsulfonyl)-1H-imidazo[4,5-b]pyrazin-6-yl)thio)pyridin-2-amine

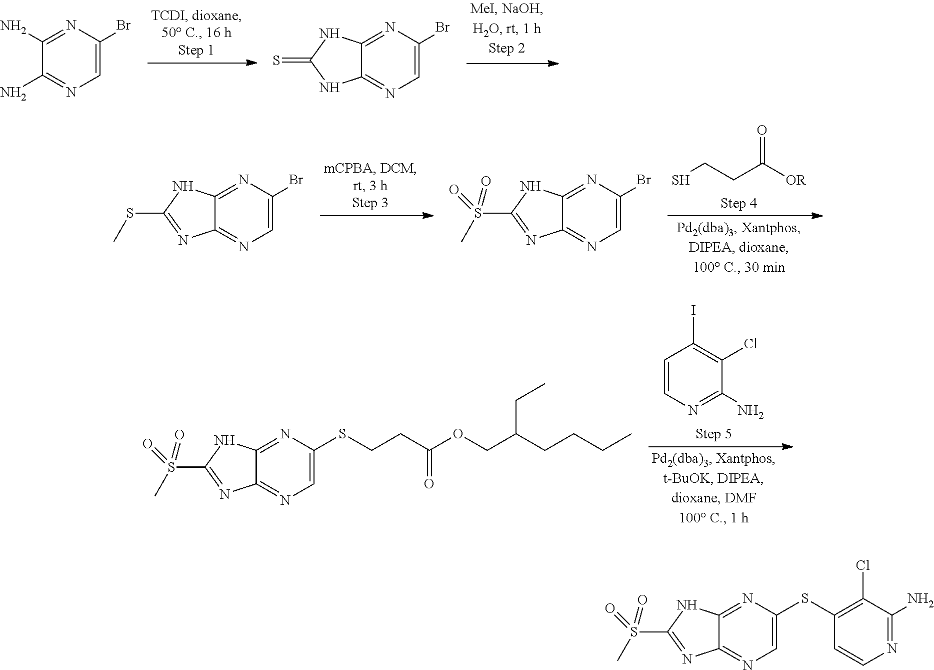

### Step 1: 5-bromo-1,3-dihydro-2H-imidazo[4,5-b]pyrazine-2-thione

A solution of 5-bromopyrazine-2,3-diamine (5.0 g, 26.5 mmol) in 1,4-dioxane (55 mL) was treated with TCDI (6.6 g, 37.0 mmol). The mixture was heated at 50° C. for 16 h. After cooling, the reaction mixture was concentrated in vacuo and purified by flash chromatography (0-50% EtOAc in petroleum ether) to give the title compound as a yellow solid (5.4 g, 79%). ¹H NMR (400 MHz, DMSO-d₆) δ 13.62 (br s, 2H), 8.22 (s, 1H). LCMS (ES⁺) Method 2: m/z 231, 233 (M+H)⁺, RT 0.87 min.

### Step 2: 6-bromo-2-(methylthio)-1H-imidazo[4,5-b]pyrazine

A solution of 5-bromo-1,3-dihydro-2H-imidazo[4,5-b]pyrazine-2-thione (3.0 g, 13.0 mmol) in H₂O (85 mL) at rt was treated with NaOH (799 mg, 19.5 mmol) and stirred at this temperature until complete dissolution of the starting material. Mel (1.2 mL, 19.5 mmol) was added, and the reaction mixture was stirred at rt for 1 h. A solution of NaOH 2N was added until clearness and the aqueous solution was washed with CHCl₃, concentrated, and acidified to pH 6.5 with aqueous HCl 6N. The resulting precipitate was filtered off and washed with H₂O to give the title compound as a pale yellow solid (1.8 g, 56%). ¹H NMR (300 MHz, DMSO-d₆) δ 7.76 (s, 1H), 2.55 (s, 3H). LCMS (ES⁺) Method 2: m/z 245, 247 (M+H)⁺, RT 1.03 min.

### Step 3: 6-bromo-2-(methylsulfonyl)-1H-imidazo[4,5-b]pyrazine

m-CPBA (4.2 g, 18.4 mmol) was added portionwise to a solution of 6-bromo-2-(methylthio)-1H-imidazo[4,5-b]pyrazine (1.8 g, 7.3 mmol) in DCM (70 mL) at 0° C., then the resulting reaction mixture was stirred at rt for 3 h. After addition of aqueous HCl 6N (10 mL) the mixture was extracted with a solution of CHCl₃/EtOH 95:5 (3×150 mL). The organic phase was washed with H₂O and brine. The dried organics were concentrated in vacuo and the residue was purified by flash chromatography (C18; 0-50% MeCN/H₂O+0.1% TFA) to give the title compound as a white solid (1.5 g, 75%). ¹H NMR (400 MHz, DMSO-d₆) δ 8.79 (s, 1H), 3.56 (s, 3H). LCMS (ES⁺) Method 2: m/z 277, 279 (M+H)⁺, RT 0.86 min.

### Step 4: 2-ethylhexyl 3-((2-(methylsulfonyl)-1H-imidazo[4,5-b]pyrazin-6-yl)thio)propanoate

A pressure tube was charged with 6-bromo-2-(methylsulfonyl)-1H-imidazo[4,5-b]pyrazine (800 mg, 2.9 mmol), 2-ethylhexyl 3-mercaptopropanoate (722 uL, 3.2 mmol), DIPEA (1.0 mL, 5.8 mmol), XantPhos (83 mg, 0.14 mmol) and Pd₂(dba)₃ (66 mg, 0.07 mmol) in 1,4-dioxane (15 mL). The mixture was degassed with N₂ for 1 min and heated at 100° C. for 30 min. After cooling, the mixture was concentrated in vacuo and purified by flash chromatography (10-100% EtOAc+10% MeOH in petroleum ether) to give the title compound as a pale-yellow solid (993 mg, 75%). LCMS (ES⁺) Method 2: m/z 415 (M+H)⁺, RT 2.18 min.

### Step 5: 3-chloro-4-((2-(methylsulfonyl)-1H-imidazo[4,5-b]pyrazin-6-yl)thio)pyridin-2-amine

A solution of 2-ethylhexyl 3-((2-(methylsulfonyl)-1H-imidazo[4,5-b]pyrazin-6-yl)thio)propanoate (100 mg, 0.24 mmol), 3-chloro-4-iodopyridin-2-amine (73.7 mg, 0.29 mmol), XantPhos (6.9 mg, 0.012 mmol), Pd₂(dba)₃ (5.5 mg, 0.006 mmol) were dissolved in 1,4-dioxane (1.4 mL) and DMF (1.0 mL) under N₂ flux, then potassium 2-methylpropan-2-olate (40.6 mg, 0.36 mmol) and DIPEA (0.08 mL, 0.48 mmol) were added and stirred at 100° C. for 1 h. After cooling, the mixture was concentrated in vacuo and purified by flash chromatography (5-100% EtOAc+20% MeOH in EtOAc) to give the title compound as a yellow solid (75 mg, 87%). LCMS (ES⁺) Method 2: m/z 356 (M+H)⁺, RT 0.73 min.

### Intermediate 51: benzyl (((1S,6R,7R)-7-(2-fluorophenyl)-3-azabicyclo[4.1.0]heptan-7-yl)methyl)carbamate hydrochloride

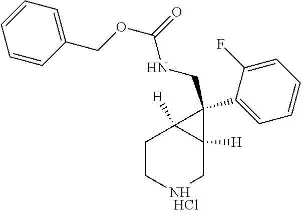 [SMILES: O=C(NC[C@]1(c2ccccc2F)[C@@H]2CCNC[C@@H]21)OCc1ccccc1 ; Cl]

The title compound was synthesized using the procedure reported for Intermediate 39 with the corresponding starting materials (step 4 was performed using benzyl(2,5-dioxopyrrolidin-1-yl)carbonate in THF/H₂O while step 5 was performed using DCM as co-solvent) and obtained as a white solid. LCMS (ES⁺) Method 2: m/z 355 (M+H)⁺, RT 1.20 min.

### Intermediate 52: benzyl (((1S,6R,7R)-7-(2,6-difluorophenyl)-3-azabicyclo[4.1.0]heptan-7-yl)methyl)carbamate hydrochloride

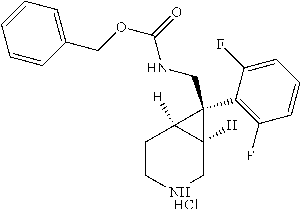 [SMILES: O=C(NC[C@]1(c2c(F)cccc2F)[C@@H]2CCNC[C@@H]21)OCc1ccccc1 ; Cl]

The title compound was synthesized using the procedure reported for Intermediate 39 with the corresponding starting materials (the step 4 was performed using benzyl(2,5-dioxopyrrolidin-1-yl)carbonate in THF/H₂O while the step 5 was performed using DCM as co-solvent) and obtained as a white solid. LCMS (ES⁺) Method 2: m/z 373 (M+H)⁺, RT 1.27 min.

### Intermediate 53: 2,4-dimethyl-5-(4,4,5,5-tetramethyl-1,3,2-dioxaborolan-2-yl)-2H-indazole

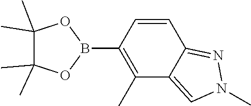

The title compound was synthesized using the procedure reported for Intermediate 45 starting from step 2 using 5-bromo-4-methyl-1H-indazole (the step 3 was performed at 90° C.) and obtained as a white solid. LCMS (ES⁺) Method 2: m/z 273 (M+H)⁺, RT 1.85 min.

### Intermediate 54: 7-chloro-2-methyl-6-(4,4,5,5-tetramethyl-1,3,2-dioxaborolan-2-yl)benzo[d]thiazole

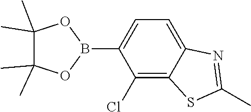

### Step 1: N-(4-bromo-3-chloro-2-fluorophenyl)acetamide

4-Bromo-3-chloro-2-fluoroaniline (7.3 g, 13.4 mmol) was dissolved in dry DCM (27.0 mL) and DIPEA (5.84 mL, 33.4 mmol) was added. The mixture was cooled in an ice bath and Ac₂O (2.27 mL, 24.1 mmol) was slowly added. The reaction was warmed to rt and stirred for 18 h. After completion the reaction was washed with HCl 1M, NaHCO₃ sat. sol. and H₂O. The organic phase was concentrated under reduced pressure to afford the title compound (3.16 g, 88%) as an off-white crystal. LCMS (ES⁺): m/z 266 (M+H)⁺.

### Step 2: 6-bromo-7-chloro-2-methylbenzo[d]thiazole

N-(4-bromo-3-chloro-2-fluorophenyl)acetamide (3.16 g, 11.9 mmol) was dissolved in dry toluene (47.5 mL) and Lawesson's reagent (2.87 g, 7.1 mmol) was added. The reaction mixture was heated at 110° C. for 3 h. Cs₂CO₃ (7.72 g, 23.7 mmol) was added, and the mixture was stirred at 110° C. for 3 h. After completion, the reaction mixture was diluted with H₂O and the aqueous phase was further extracted with toluene (3×). The combined organic phases were dried and concentrated. The crude product was purified by flash chromatography (0-40% EtOAc in n-heptane) to afford the title product (1.85 g, 59%). LCMS (ES⁺): m/z 262 (M+H)⁺.

### Step 3: 7-chloro-2-methyl-6-(4,4,5,5-tetramethyl-1,3,2-dioxaborolan-2-yl)benzo[d]thiazole

6-bromo-7-chloro-2-methylbenzo[d]thiazole (1.85 g, 7.0 mmol), B₂pin₂ (2.32 g, 9.2 mmol), Pd(dppf)Cl₂·DCM (515.6 mg, 0.7 mmol) and KOAc (1.38 g, 14.1 mmol) were suspended in 1,4-dioxane (35.0 mL) and heated to 110° C. for 4 h. After completion, the mixture was cooled to rt, and the solvent was concentrated. The crude product was purified by flash chromatography (25-40% EtOAc in n-hexane) to afford the title product (1.03 g, 47%) as an off-white solid. ¹H NMR (500 MHz, DMSO-d₆) δ 7.84 (d, J=8.1 Hz, 1H), 7.72 (d, J=8.1 Hz, 1H), 2.80 (s, 3H), 1.30 (s, 12H). LCMS (ES⁺): m/z 310 (M+H)⁺.

### Intermediate 55: 4-chloro-2,3-dimethyl-5-(4,4,5,5-tetramethyl-1,3,2-dioxaborolan-2-yl)-2H-indazole

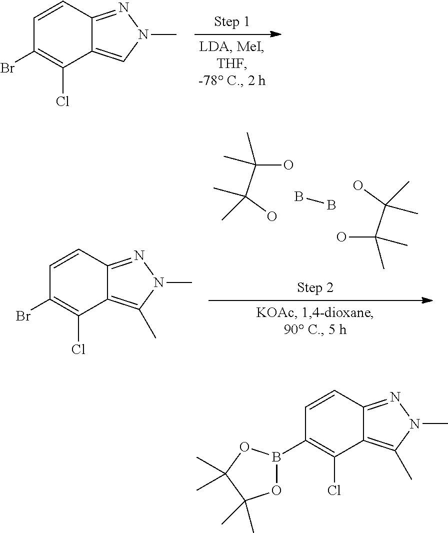

### Step 1: 5-bromo-4-chloro-2,3-dimethyl-2H-indazole

A solution of 5-bromo-4-chloro-2-methyl-2H-indazole (prepared as described in the synthesis of Intermediate 45; 300 mg, 1.22 mmol) in THF (6 mL) was treated with LDA (2M in THF; 0.79 mL, 1.59 mmol) at −78° C. The mixture was warmed to 0° C. and stirred at this temperature for 30 min. Reaction mixture was cooled to −78° C. and Mel (0.11 mL, 1.83 mmol) was added, and the mixture was stirred at −78° C. for 2 h, then quenched with NH₄C₁ sat. sol. and the mixture extracted with EtOAc. The organic layer was washed with brine, dried over Na₂SO₄, filtered, and concentrated in vacuo. The crude product was purified by flash chromatography (0-50% EtOAc in petroleum ether) to afford the title compound as a yellow solid (215 mg, 68%). LCMS (ES⁺) Method 2: m/z 259,261 (M+H)⁺, RT 1.79 min.

### Step 2: 4-chloro-2,3-dimethyl-5-(4,4,5,5-tetramethyl-1,3,2-dioxaborolan-2-yl)-2H-indazole

A suspension of Pd(dppf)Cl₂·DCM (68.49 mg, 0.080 mmol), 5-bromo-4-chloro-2,3-dimethyl-2H-indazole (215 mg, 0.830 mmol), B₂pin₂ (336.6 mg, 1.33 mmol), and KOAc (203.3 mg, 2.07 mmol) in degassed 1,4-dioxane (2.5 mL) was heated at 90° C. for 5 h. After completion, the mixture was cooled to rt, the solid precipitate was filtered off and washed with EtOAc. The filtrate was concentrated in vacuo. The crude residue was purified by flash chromatography (0-50% EtOAc in petroleum ether) to afford the title compound as a white off solid (200 mg, 79%). ¹H NMR (400 MHz, DMSO-d₆) δ 7.42 (d, J=8.8 Hz, 1H), 7.34 (d, J=8.8 Hz, 1H), 4.05 (s, 3H), 2.81 (s, 3H), 1.31 (s, 12H). LCMS (ES⁺) Method 2: m/z 307 (M+H)⁺, RT 1.90 min.

### Intermediate 56: 4-chloro-3-methoxy-2-methyl-5-(4,4,5,5-tetramethyl-1,3,2-dioxaborolan-2-yl)-2H-indazole

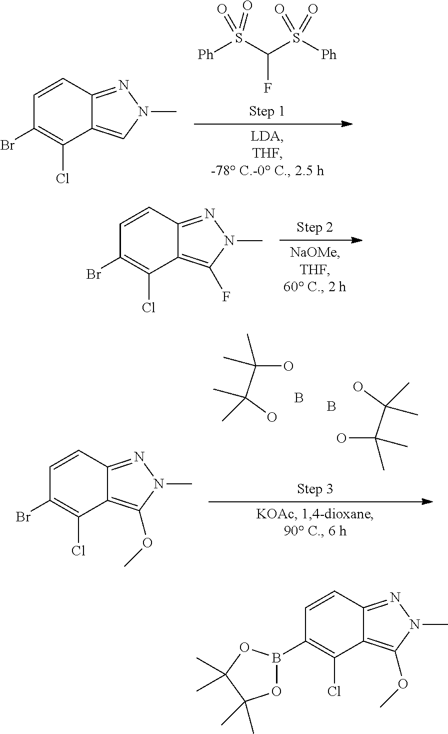

### Step 1: 5-bromo-4-chloro-3-fluoro-2-methyl-2H-indazole

A solution of 5-bromo-4-chloro-2-methyl-2H-indazole (prepared as described in the synthesis of Intermediate 45; 490 mg, 2 mmol) in THF (9 mL) was treated with LDA (2 M in THF; 1.3 mL, 2.59 mmol) at −78° C. and the mixture was stirred at 0° C. for 30 min. (fluoromethylenedisulfonyl)dibenzene (0.41 mL, 2.99 mmol) was added and mixture stirred at 0° C. for 2 h. The reaction was quenched with H₂O and extracted with EtOAc. The organic layer was washed with brine, dried over Na₂SO₄, filtered, and concentrated in vacuo. The crude residue was purified by flash chromatography (0-40% EtOAc in petroleum ether) to afford the title compound as a yellow solid (250 mg, 48%). ¹H NMR (400 MHz, DMSO-d₆) δ 7.49-7.42 (m, 2H), 4.03 (s, 3H). LCMS (ES⁺) Method 2: m/z 263-265 (M+H)⁺, RT 1.84 min.

### Step 2: 5-bromo-4-chloro-3-methoxy-2-methyl-2H-indazole

A solution of 5-bromo-4-chloro-3-fluoro-2-methyl-2H-indazole (250 mg, 0.95 mmol) in THF (4.5 mL) was treated with NaOMe (25% wt in MeOH; 0.65 mL, 2.85 mmol) and the resulting mixture was stirred at 60° C. for 2 h. After cooling mixture was poured in H₂O and extracted with EtOAc. The organic layer was washed with brine, dried over Na₂SO₄, filtered, and concentrated in vacuo. The crude residue was purified by flash chromatography (0-40% EtOAc in petroleum ether) to afford the title compound as a yellow solid (195 mg, 75%). ¹H NMR (400 MHz, DMSO-d₆) δ 7.44-7.39 (m, 2H), 4.07 (s, 3H), 3.98 (s, 3H). LCMS (ES⁺) Method 2: m/z 275-277 (M+H)⁺, RT 1.75 min.

### Step 3: 4-chloro-3-methoxy-2-methyl-5-(4,4,5,5-tetramethyl-1,3,2-dioxaborolan-2-yl)-2H-indazole

A solution of B₂pin₂ (287.6 mg, 1.13 mmol), Pd(PPh₃)₂Cl₂·DCM (58.5 mg, 0.070 mmol), 5-bromo-4-chloro-3-methoxy-2-methyl-2H-indazole (195 mg, 0.710 mmol) and KOAc (173.6 mg, 1.77 mmol) in degassed 1,4-dioxane (2.5 mL) was heated at 90° C. for 6 h. After completion, the mixture was cooled to rt, the solid precipitate was filtered off and washed with EtOAc. The filtrate was concentrated in vacuo. The crude product was purified by flash chromatography (0-50% EtOAc in petroleum ether) to afford the title compound as a white off solid (233 mg, 65% pure, 66%). LCMS (ES⁺) Method 2: m/z 323 (M+H)⁺, RT 1.92 min.

### Intermediate 57: 6-chloro-3-iodo-5-methyl-1-(tetrahydro-2H-pyran-2-yl)-1H-pyrazolo[3,4-b]pyrazine

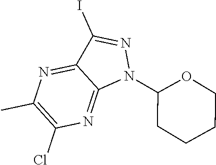 [SMILES: Cc1nc2c(I)nn(C3CCCCO3)c2nc1Cl]

### Step 1: 3,5-dichloropyrazine-2-carbonyl chloride

A mixture of 3,5-dichloropyrazine-2-carboxylic acid (10.0 g, 51.8 mmol) and SOCl₂ (34 mL, 466.3 mmol) was heated at 90° C. for 16 h. After cooling, the dark solution was evaporated to dryness under reduced pressure. The crude oil was dissolved in toluene (2 mL) and evaporated to dryness to afford the title compound (11.0 g, quantitative). TLC (CHCl₃/MeOH=20:1), Rf: 0.95.

### Step 2: 3,5-dichloro-N-methoxy-N-methylpyrazine-2-carboxamide

N,O-Dimethylhydroxylamine hydrochloride (5.3 g, 54.4 mmol) was dissolved in DCM (272 mL) then DIPEA (17.3 mL, 98.9 mmol) and 3,5-dichloropyrazine-2-carbonyl chloride (11.0 g, 49.4 mmol) in DCM (49 mL) were added. The resultant mixture was stirred at rt for 2 h. After completion, the mixture was diluted with DCM and washed with H₂O (3×). The organic phase was dried over Na₂SO₄ filtered and concentrated to afford the title product (10.9 g, 93%). LCMS (ES⁺): m/z 236 (M+H)⁺.

### Step 3: 3,5-dichloropyrazine-2-carbaldehyde

Under argon atmosphere 3,5-dichloro-N-methoxy-N-methylpyrazine-2-carboxamide (10.9 g, 46.2 mmol) was dissolved in dry THF (218 mL) and cooled to −78° C. DIBAL-H (1M solution in cyclohexane, 50.8 mL, 50.8 mmol) was added slowly and stirred at −78° C. for 2 h. After completion, 1M HCl sol. (71 mL) was added, and the reaction mixture was allowed to warm to 0° C. The mixture was stirred at this temperature for 20 min then extracted with EtOAc (2×). The organic phase was dried over Na₂SO₄ filtered and concentrated to afford the crude product (9.68 g, quantitative) as a brown oil. ¹H NMR (500 MHz, CDCl₃): δ 10.29 (s, 1H), 8.71 (s, 1H).

### Step 4: 6-chloro-1H-pyrazolo[3,4-b]pyrazine

3,5-dichloropyrazine-2-carbaldehyde (4.79 g, 27.1 mmol) was dissolved in NMP (26.0 mL) under argon atmosphere and N₂H₄·H₂O (2.63 mL, 54.1 mmol) was added dropwise. The mixture was stirred at 70° C. for 2 h. After completion, the reaction was cooled to rt and poured into 1M HCl sol. and taken up in EtOAc.

The organic phase was washed with brine, dried over Na₂SO₄, filtered, and concentrated in vacuo to afford the title product (1.56 g, 37%) as a brownish yellow solid. LCMS (ES⁺): m/z 153 (M−H)⁻.

### Step 5: 6-chloro-3-iodo-1H-pyrazolo[3,4-b]pyrazine

6-chloro-1H-pyrazolo[3,4-b]pyrazine (1.56 g, 10.1 mmol) was dissolved in dry DMF (25.0 mL) and NIS (3.4 g, 15.1 mmol) was added. The resultant mixture was stirred at rt overnight. After completion, H₂O was added, and the precipitated solid was filtered off. The product was washed with 5% aq. Na₂S₂O₃ sol., H₂O, filtered and dried to afford the title product (2.1 g, 74%). LCMS (ES⁺): m/z 279 (M−H)⁻.

### Step 6: methyl 6-chloro-3-iodo-1H-pyrazolo[3,4-b]pyrazine-1-carboxylate

A solution of NaH (60% in mineral oil; 427.8 mg, 10.7 mmol) in dry DMF (20 mL) was cooled to 0° C., then 6-chloro-3-iodo-1H-pyrazolo[3,4-b]pyrazine (2.0 g, 7.1 mmol) was added. The reaction mixture was allowed to warm to rt and stirred for 2.5 h. Then, the mixture was cooled to 0-10° C. and methyl chloroformate (1.65 mL, 21.4 mmol) was added and stirring was continued for 20 min. After completion, the mixture was poured in H₂O (17 mL). The solid precipitate was filtered off, washed with H₂O, and dried to afford the title product (1.73 g, 72%). LCMS (ES⁺): m/z 339 (M+H)⁺.

### Step 7: methyl 6-chloro-3-iodo-5-methyl-1H-pyrazolo[3,4-b]pyrazine-1-carboxylate

Methyl 6-chloro-3-iodo-1H-pyrazolo[3,4-b]pyrazine-1-carboxylate (1.63 g, 4.8 mmol) and APS (3.29 g, 14.4 mmol) were dissolved in MeCN (14.5 mL) and H₂O (7 mL). A solution of AcOH (1.38 mL, 24.1 mmol) and AgNO₃ (8.18 g, 48.2 mmol) in MeCN (3.3 mL) and H₂O (1.6 mL) was then added dropwise at 70° C. The resultant mixture was stirred at 70° C. for 10 min. After completion the mixture was cooled to rt, and its pH was adjusted to 8 with NaHCO₃ sat. sol. The mixture was filtered, and the filtrate was extracted with DCM (3×). The organic phase was dried to afford the title product (1.1 g, 65%). LCMS (ES⁺): m/z 353 (M+H)⁺.

### Step 8: 6-chloro-3-iodo-5-methyl-1-(tetrahydro-2H-pyran-2-yl)-1H-pyrazolo[3,4-b]pyrazine

Methyl 6-chloro-3-iodo-5-methyl-1H-pyrazolo[3,4-b]pyrazine-1-carboxylate (1.1 g, 3.1 mmol) was dissolved in DCM (15.5 mL) and piperidine (340 uL, 3.43 mmol) was added. The mixture was stirred at rt for 2 h. After completion PTSA (771.6 mg, 4.1 mmol) and DHP (314 μL, 3.4 mmol) were added and stirring was continued at rt for 1 h. After completion the mixture was extracted with NaHCO₃ sat. sol. and brine. The organic phase was dried, concentrated and the crude product was purified by flash chromatography (0-40% EtOAc in n-heptane) to afford the title product (550 mg, 46%) as a pale-yellow solid. ¹H NMR (500 MHz, DMSO-d₆): δ 5.85 (dd, J=10.2, 1.8 Hz, 1H), 3.89 (d, J=11.5 Hz, 1H), 3.74-3.64 (m, 1H), 2.69 (s, 3H), 2.44-2.32 (m, 1H), 1.95 (dd, J=13.0, 37.1 Hz, 2H), 1.80-1.68 (m, 1H), 1.62-1.49 (m, 2H). LCMS (ES: m/z 295 (M+H)⁺.

### Intermediate 58: 8-chloro-2-methoxy-7-(4,4,5,5-tetramethyl-1,3,2-dioxaborolan-2-yl)quinoxaline

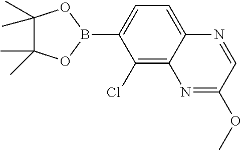 [SMILES: COc1cnc2ccc(B3OC(C)(C)C(C)(C)O3)c(Cl)c2n1]

### Step 1: 4-Bromo-3-chloro-2-nitroaniline

3-Chloro-2-nitroaniline (3.0 g, 17.4 mmol) was dissolved in glacial AcOH (155.0 mL) and NBS (3.1 g, 17.4 mmol) was added. The mixture was stirred at reflux for 3 h. Upon completion, the reaction was cooled to rt, and poured into ice H₂O to give a precipitate that was filtered, washed with H₂O, and dried to afford the title product (3.37 g, 77%) as a brown solid. ¹H NMR (300 MHz, CDCl₃): δ 7.46 (d, J=8.9 Hz, 1H), 6.64 (d, J=8.9 Hz, 1H), 4.76 (s, 2H).

### Step 2: tert-butyl (4-bromo-3-chloro-2-nitrophenyl)(tert-butoxycarbonyl)carbamate

4-bromo-3-chloro-2-nitroaniline (3.37 g, 13.4 mmol) was dissolved in THF (27.0 mL) and (Boc)₂O (7.7 mL, 33.5 mmol) and DMAP (327.6 mg, 2.68 mmol) were added. The mixture was stirred at rt overnight. After completion the solvent was evaporated under reduced pressure and the crude product was purified by flash chromatography (0-30% EtOAc in cyclohexane) to afford the title product (4.7 g, 77%) as an off-white solid. LCMS (ES⁺): m/z 473 (M+H+Na)⁺.

### Step 3: tert-butyl (4-bromo-3-chloro-2-nitrophenyl)carbamate

tert-butyl N-(4-bromo-3-chloro-2-nitrophenyl)-N-[(tert-butoxy)carbonyl]carbamate (4.7 g, 10.4 mmol) was dissolved in DCM (21.0 mL) and TFA (1.6 mL, 20.8 mmol) was added. The mixture was stirred at rt for 3 h. After completion the mixture was quenched with NaHCO₃ sat. sol. and extracted with DCM (3×). The organic layer was dried over anhydrous Na₂SO₄, filtered, and concentrated in vacuo to afford the title compound (3.48 g, 95%) as a yellow solid. ¹H NMR (300 MHz, DMSO-d₆): δ 9.68 (br s, 1H), 7.92 (d, J=9.0 Hz, 1H), 7.53 (d, J=9.0 Hz, 1H), 1.41 (s, 9H); LCMS (ES⁺): m/z 304 (M+H—NO₂)⁺.

### Step 4: Ethyl N-(4-bromo-3-chloro-2-nitrophenyl)-N-(tert-butoxycarbonyl)glycinate

tert-Butyl N-(4-bromo-3-chloro-2-nitrophenyl)carbamate (3.48 g, 9.9 mmol) was dissolved in dry DMF (30 mL) and Cs₂CO₃ (9.68 g, 29.7 mmol) was added. The mixture was stirred at rt for 10 min then ethyl bromoacetate (1.97 mL, 17.8 mmol) was slowly added. The reaction mixture was stirred at rt for 1.5 h. After completion the mixture was diluted with H₂O and extracted with EtOAc (3×). The organic phase was dried and concentrated. The crude product was purified by flash chromatography (0-40% EtOAc in cyclohexane) to afford the title product (2.49 g, 57%) as an orange solid. TLC (n-heptane-EtOAc=3:2), Rf 0.8.

### Step 5: Ethyl (4-bromo-3-chloro-2-nitrophenyl)glycinate

Ethyl N-(4-bromo-3-chloro-2-nitrophenyl)-N-(tert-butoxycarbonyl)glycinate (2.48 g, 5.68 mmol) was dissolved in 1,4-dioxane (17 mL) and HCl in 1,4-doxane (4N; 17.0 mL) was added. The mixture was stirred at rt overnight. After completion, the solvent was evaporated under reduced pressure. The crude product was diluted with DCM and evaporated again (×3) to afford the title product (1.83 g, 96%) as an orange solid (HCl salt). TLC (n-heptane-EtOAc=3:2)=Rf: 0.7.

### Step 6: 7-Bromo-8-chloro-3,4-dihydroquinoxalin-2(1H)-one

Ethyl (4-bromo-3-chloro-2-nitrophenyl)glycinate (890 mg, 2.64 mmol), Fe (1.47 g, 26.36 mmol) and NH₄Cl (1.41 g, 26.36 mmol) were suspended in EtOH (18.5 mL) and the resultant mixture was stirred at reflux overnight. After completion, AcOH (7.99 mL) was added, and the mixture was stirred at reflux overnight and then diluted with H₂O and washed with CHCl₃ (3×). The organic phase was dried over Na₂SO₄ filtered and concentrated to afford the title product (645 mg, 93%) as a brown solid. LCMS (ES⁺): m/z 261 (M+H)⁺.

### Step 7: 7-Bromo-8-chloroquinoxalin-2-ol

7-Bromo-8-chloro-3,4-dihydroquinoxalin-2(1H)-one (645 mg, 2.45 mmol) was suspended in aq. NaOH (235 mg, 5.88 mmol), H₂O₂(0.5 ml, 5.6 mmol, 30 wt % solution) and H₂O (4.9 mL). The mixture was stirred at reflux for 3 h. After completion the mixture was cooled to rt and AcOH (2.45 mL) was added. The precipitated solid was filtered off, washed with H₂O, and dried to afford the title product (74%) as a brown solid. LCMS (ES⁺): m/z 259 (M+H)⁺.

### Step 8: 7-Bromo-8-chloro-2-methoxyquinoxaline

7-Bromo-8-chloroquinoxalin-2-ol (822 mg, 2.53 mmol) and K₂CO₃ (525.3 mg, 3.80 mmol) were suspended in dry DMF (25 mL) then Mel (173 μL, 2.78 mmol) was added and the reaction mixture was stirred at rt for 3 h. After completion H₂O was added and a solid precipitate was collected by filtration, washed with H₂O, and dried. The crude product was purified by flash chromatography (0-25% EtOAc in n-heptane) to afford the title product (368 mg, 53%). LCMS (ES⁺): m/z 273 (M+H)⁺.

### Step 9: 8-Chloro-2-methoxy-7-(4,4,5,5-tetramethyl-1,3,2-dioxaborolan-2-yl)quinoxaline

7-Bromo-8-chloro-2-methoxyquinoxaline (368 mg, 1.34 mmol), B₂pin₂ (444.2 g, 1.75 mmol), Pd(dppf)Cl₂-DCM (98.5 mg, 0.14 mmol) and KOAc (264.1 mg, 2.69 mmol) were suspended in 1,4-dioxane (6.7 mL) and the reaction mixture was heated at 110° C. for 4 h. After completion, the reaction mixture was cooled to rt, filtered, and washed with EtOAc. The filtrate was concentrated, and the residue was dissolved in toluene followed by n-heptane until precipitation occurred. The suspension was filtered, and the filtrate was concentrated to afford the crude title compound (569 mg, quantitative) as a dark brown crystal. ¹H NMR (500 MHz, DMSO-d₆): δ 8.67 (s, 1H), 7.92 (d, J=8.2 Hz, 1H), 7.72 (d, J=8.2 Hz, 1H), 4.07 (s, 3H), 1.34 (s, 12H). LCMS (ES⁺): m/z 321 (M+H)⁺.

### Intermediate 59: 5-bromoquinoline-8-sulfonyl fluoride

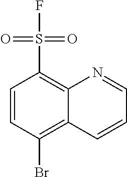 [SMILES: O=S(=O)(F)c1ccc(Br)c2cccnc12]

### Step 1: 5-bromoquinoline-8-sulfonyl fluoride

To a suspension of 5-bromoquinoline-8-sulfonyl chloride (122 mg, 0.40 mmol) in MeCN (1 mL) was added a solution of KHF₂ (71.5 mg, 0.92 mmol) in H₂O (0.5 mL) at rt. The reaction mixture was vigorously stirred at rt for 4 h, then was diluted with EtOAc and H₂O. The organic layer was dried over Na₂SO₄, filtered, and concentrated in vacuo to obtain the crude title compound as off-white solid, that was used in the next step without further purification (104 mg, 90%). LCMS (ES⁺) Method 1A: m/z 290, 292 (M+H)⁺, RT 1.98 min.

### Intermediate 61: 68-(methylsulfonyl)-5-(4,4,5,5-tetramethyl-1,3,2-dioxaborolan-2-yl)quinoline

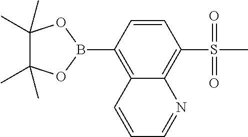

To a solution of Intermediate 59 (70 mg, 0.24 mmol) in dry THF (1.7 mL) cooled to −78° C., under N₂ flow, methylmagnesium bromide (3N solution in Et₂O, 0.2 mL, 0.60 mmol) was added dropwise. The reaction mixture was stirred at −78° C. for 30 min, then H₂O and EtOAc were added, the mixture was allowed to warm to rt, and the two phases were separated. Aqueous layer was extracted again with EtOAc, then the collected organic layer was dried over Na₂SO₄, filtered, and concentrated in vacuo to obtain the crude title compound as an off-white solid, that was used in the next step without purification (70 mg); LCMS (ES⁺) Method 1A: m/z 286, 288 (M+H)⁺, RT 1.57 min) and further used according to the procedure reported for Intermediate 15, and obtained as a brown solid. LCMS (ES⁺) Method 1A: m/z 334 (M+H)⁺, RT 1.98 min.

### Intermediate 65: benzyl (((1R,5S,6r)-6-(2-methyloxazol-4-yl)-3-azabicyclo[3.1.0]hexan-6-yl)methyl)carbamate hydrochloride

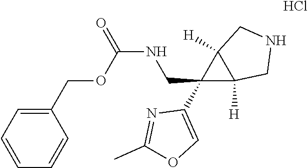 [SMILES: Cl ; Cc1nc([C@@]2(CNC(=O)OCc3ccccc3)[C@@H]3CNC[C@@H]32)co1]

The title compound was synthesized using the procedure reported for Intermediate 94 using 2-(2-methyloxazol-4-yl)acetonitrile in the step 3. LCMS (ES⁺) Method 1A: m/z 328 (M+H)⁺, RT 1.17 min.

### Intermediate 66: 8-(methoxymethyl)-5-(4,4,5,5-tetramethyl-1,3,2-dioxaborolan-2-yl)quinoline

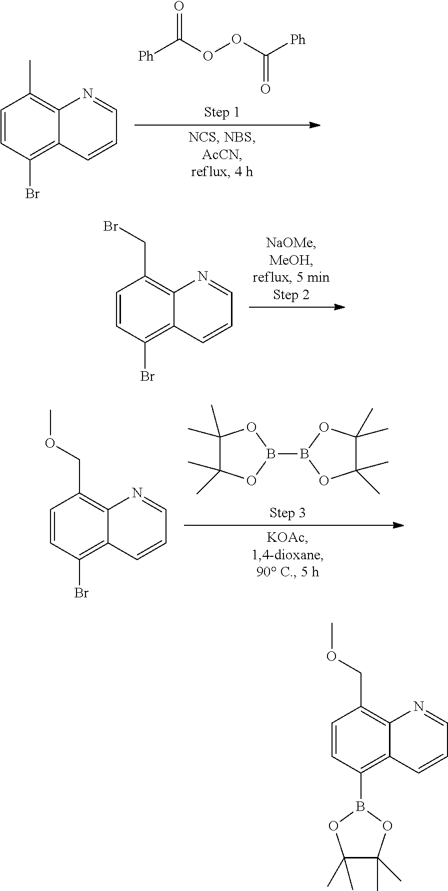

### Step 1: 5-bromo-8-(bromomethyl)quinoline

A solution of 5-bromo-8-methylquinoline (200 mg, 0.90 mmol) and 1-chloropyrrolidine-2,5-dione (120.3 mg, 0.90 mmol) in MeCN (3.6 mL) was treated with benzoyl peroxide (10.9 mg, 0.050 mmol) and the reaction mixture was stirred at reflux for 2 h. NBS (75.0 mg, 0.42 mmol) was added, and the reaction mixture stirred for additional 2 h. After cooling the mixture was treated with H₂O and EtOAc. Phases were separated, and the organic phase was dried over Na₂SO₄, filtered, and concentrated in vacuo. The crude residue was purified by flash chromatography (0-100% EtOAc in petroleum ether) to afford the title compound as a white solid (191 mg, 70%). ¹H NMR (400 MHz, DMSO-d₆) δ 9.10-9.08 (m, 1H), 8.56 (d, J=8.3 Hz, 1H), 8.00 (d, J=7.9 Hz, 1H), 7.90 (d, J=7.9 Hz, 1H), 7.79-7.76 (m, 1H), 5.28 (s, 2H). LCMS (ES⁺) Method 2: m/z 300-302-304 (M+H)⁺, RT 2.12 min.

### Step 2: 5-bromo-8-(methoxymethyl)quinoline

A suspension of 5-bromo-8-(bromomethyl)quinoline (50 mg, 0.17 mmol) in MeOH (0.55 mL) was treated with NaOMe (25% wt in MeOH; 0.17 mL, 0.18 mmol) and the resulting mixture was stirred at reflux for 5 min. After cooling, the mixture was poured in H₂O and extracted with DCM. The organic layer was separated and concentrated in vacuo to afford the title compound as a white solid (40 mg, 96%). ¹H NMR (400 MHz, DMSO-d₆) δ 8.99 (dd, J=4.2, 1.3 Hz, 1H), 8.53 (dd, J=8.6, 1.5 Hz, 1H), 7.98 (d, J=7.9 Hz, 1H), 7.75-7.69 (m, 2H), 5.05 (s, 2H), 3.31 (s, 3H). LCMS (ES⁺) Method 2: m/z 250-252 (M+H)⁺, RT 1.85 min.

### Step 3: 8-(methoxymethyl)-5-(4,4,5,5-tetramethyl-1,3,2-dioxaborolan-2-yl)quinoline

A solution of B₂pin₂ (64.5 mg, 0.25 mmol), Pd(dppf)Cl₂·DCM (13.1 mg, 0.02 mmol), 5-bromo-8-(methoxymethyl)quinoline (40 mg, 0.16 mmol) and KOAc (38.9 mg, 0.40 mmol) in degassed 1,4-dioxane (0.5 mL) was heated at 90° C. for 5 h. After completion, the mixture was cooled to rt, the solid precipitate was filtered off and washed with EtOAc. The filtrate was concentrated in vacuo. The crude product was purified by flash chromatography (0-50% EtOAc in petroleum ether) to afford the title compound as a white off solid (30 mg, 63%). LCMS (ES⁺) Method 2: m/z 300 (M+H)⁺, RT 1.95 min.

### Intermediate 67: benzyl (((1R,5S,6s)-6-(2-fluorophenyl)-3-azabicyclo[3.1.0]hexan-6-yl)methyl)carbamate hydrochloride

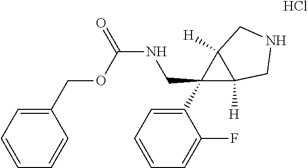 [SMILES: Cl ; O=C(NC[C@]1(c2ccccc2F)[C@@H]2CNC[C@@H]21)OCc1ccccc1]

Intermediate 67 was synthesized using the procedure reported for Intermediate 94 using 2-(2-fluorophenyl)acetonitrile in the step 3. LCMS (ES⁺) Method 1A: m/z 341 (M+H)⁺, RT 1.35 min.

### Intermediate 68: 8-(difluoromethyl)-5-(4,4,5,5-tetramethyl-1,3,2-dioxaborolan-2-yl)quinoline

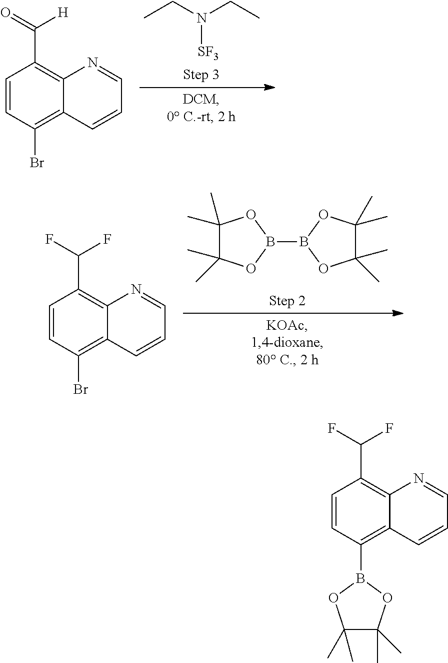

### Step 1: 5-bromo-8-(difluoromethyl)quinoline

A solution of 5-bromoquinoline-8-carbaldehyde (200 mg, 0.85 mmol) in DCM (4.2 mL) was treated with N,N-diethyl-1,1,1-trifluoro-14-sulfanamine (0.11 mL, 0.85 mmol) at 0° C. The reaction mixture was warmed to rt and stirred at this temperature for 2 h before being quenched by addition of ice. Phases were separated, and the organic phase was dried over Na₂SO₄, filtered, and concentrated in vacuo to afford the title compound as an orange powder (166 mg, 76%). LCMS (ES⁺) Method 2: m/z 258, 260 (M+H)⁺, RT 2.03 min.

### Step 2: 8-(difluoromethyl)-5-(4,4,5,5-tetramethyl-1,3,2-dioxaborolan-2-yl)quinoline

A solution of KOAc (114.1 mg, 1.16 mmol), 5-bromo-8-(difluoromethyl)quinoline (100 mg, 0.390 mmol), Pd(dppf)Cl₂·DCM (32.0 mg, 0.040 mmol) and B₂pin₂ (118.1 mg, 0.470 mmol) in degassed 1,4-dioxane (0.8 mL) was stirred at 80° C. for 2 h. The mixture was cooled to rt, the solid precipitate was filtered off and washed with EtOAc. The filtrate was concentrated in vacuo to afford the title compound as a brown oil (118 mg, 99%) which was used as a crude. LCMS (ES⁺) Method 2: m/z 306 (M+H)⁺, RT 2.37 min.

### Intermediate 69: 1-(5-(4,4,5,5-tetramethyl-1,3,2-dioxaborolan-2-yl)quinolin-8-yl)ethan-1-ol

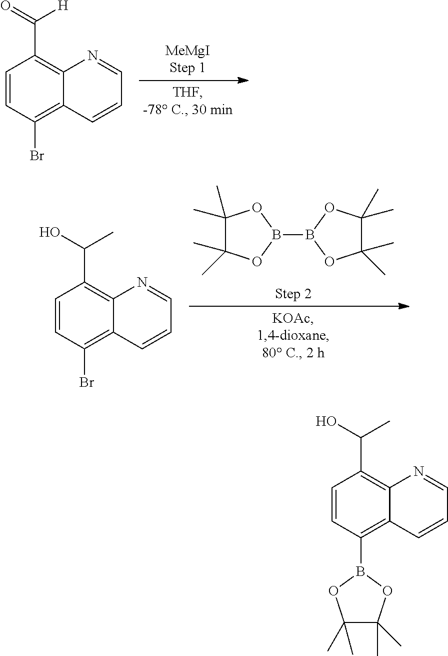

### Step 1: 1-(5-bromoquinolin-8-yl)ethan-1-ol

A solution of 5-bromoquinoline-8-carbaldehyde (200 mg, 0.85 mmol) in dry THF (4.2 mL) was treated with methyl magnesium iodide (0.34 mL, 1.02 mmol) at −78° C. The resulting mixture was stirred 30 min at this temperature before being warmed to rt. NH₄Cl sat. sol. was added, and the mixture was stirred for 10 min. The reaction mixture was then diluted with EtOAc, washed with brine and the organic phase was dried over MgSO₄, filtered, and concentrated in vacuo. The crude product was purified by flash chromatography (0-100% EtOAc in petroleum ether) to afford the title compound as an orange oil (155 mg, 73%). ¹H NMR (400 MHz, DMSO-d₆) δ 8.99 (dd, J=4.2, 1.8 Hz, 1H), 8.52 (dd, J=8.6, 1.8 Hz, 1H), 7.98 (d, J=7.9 Hz, 1H), 7.82 (d, J=7.7 Hz, 1H), 7.71 (dd, J=8.6, 4.2 Hz, 1H), 5.84-5.78 (m, 1H), 5.33 (d, J=4.4 Hz, 1H), 1.44 (d, J=6.4 Hz, 3H); LCMS (ES⁺) Method 2: m/z 250, 252 (M+H)⁺, RT 1.63 min.

### Step 2: 1-(5-(4,4,5,5-tetramethyl-1,3,2-dioxaborolan-2-yl)quinolin-8-yl)ethan-1-ol

The title compound was synthesized using the procedure reported for Intermediate 68 step 2 using 1-(5-bromoquinolin-8-yl)ethan-1-ol and obtained as a brown oil and used as a crude. LCMS (ES⁺) Method 2: m/z 300 (M+H)⁺, RT 1.65 min.

### Intermediate 70: ((1R,5S,6r)-6-(4-methylthiazol-2-yl)-3-azabicyclo[3.1.0]hexan-6-yl)methanamine

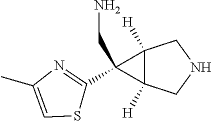

N₂H₄·H₂O (0.13 mL, 2.6 mmol) was added to a solution of Intermediate 122 (38 mg, 0.1 mmol) in EtOH (1 mL) and the reaction mixture was stirred at 40° C. for 1.5 h. The mixture was filtered, and the filtrate was evaporated under reduced pressure to afford the crude title compound as an off-white solid, that was used in the next step without further purification (30 mg). LCMS (ES⁺) Method 1A: m/z 210 (M+H)⁺, RT 0.87 min.

### Intermediate 71: 7-chloro-2-(difluoromethyl)-6-(4,4,5,5-tetramethyl-1,3,2-dioxaborolan-2-yl)-2H-indazole

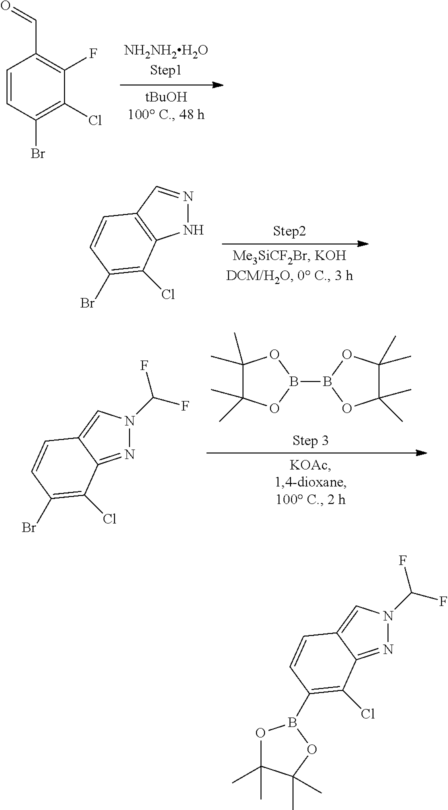

### Step 1: 6-bromo-7-chloro-1H-indazole

A mixture of 4-bromo-3-chloro-2-fluorobenzaldehyde (256 mg, 1.1 mmol) and N₂H₄H₂O (0.3 mL, 6.5 mmol) in tBuOH (5 mL) was heated at 100° C. for 48 h. Volatiles were removed under reduced pressure and the resulting crude title compound was used in the next step without further purification (249 mg). LCMS (ES⁺) Method 1A: m/z. 233, RT 1.81 min.

### Step 2: 6-bromo-7-chloro-2-(difluoromethyl)-2H-indazole

Crude 6-bromo-7-chloro-1H-indazole (249 mg, 1.1 mmol) was suspended in DCM (5 mL), cooled to 0° C. and KOH (362 mg, 6.45 mmol in 1.7 mL of H₂O) was added with vigorous stirring. Then (bromodifluoromethyl)trimethylsilane (0.4 mL, 2.15 mmol) was added and the reaction mixture was vigorously stirred at 0° C. for 3 h. The reaction was diluted with DCM and H₂O, then the organic layer was dried over Na₂SO₄, filtered, and concentrated in vacuo. The resulting crude was purified by flash chromatography on silica gel (from 0-20% EtOAc in petroleum ether) to obtain the title compound as white solid (40 mg, 13% over two steps). ¹H NMR (300 MHz, DMSO-d₆) δ 9.09 (s, 1H), 8.22 (t, J=59.9 Hz, 1H), 7.78 (d, J=9.0 Hz, 1H), 7.44 (d, J=8.9 Hz, 1H); LCMS (ES⁺) Method 1A, RT 2.01 min.

### Step 3: 7-chloro-2-(difluoromethyl)-6-(4,4,5,5-tetramethyl-1,3,2-dioxaborolan-2-yl)-2H-indazole

A mixture of B₂pin₂ (72 mg, 0.28 mmol), 6-bromo-7-chloro-2-(difluoromethyl)-2H-indazole (40 mg, 0.14 mmol), KOAc (42 mg, 0.43 mmol) Pd(dppf)Cl₂·DCM (14 mg, 0.02 mmol) in degassed 1,4-dioxane (1 mL) was heated at 100° C. for 1.5 h. The black suspension was diluted with EtOAc, filtered over a pad of celite and concentrated under reduced pressure to give the crude compound, that was used in the next step without further purification. LCMS (ES⁺) Method 1A: m/z 328 (M+H)⁺, RT 2.16 min.

### Intermediate 72: 8-isopropoxy-5-(4,4,5,5-tetramethyl-1,3,2-dioxaborolan-2-yl)quinoline

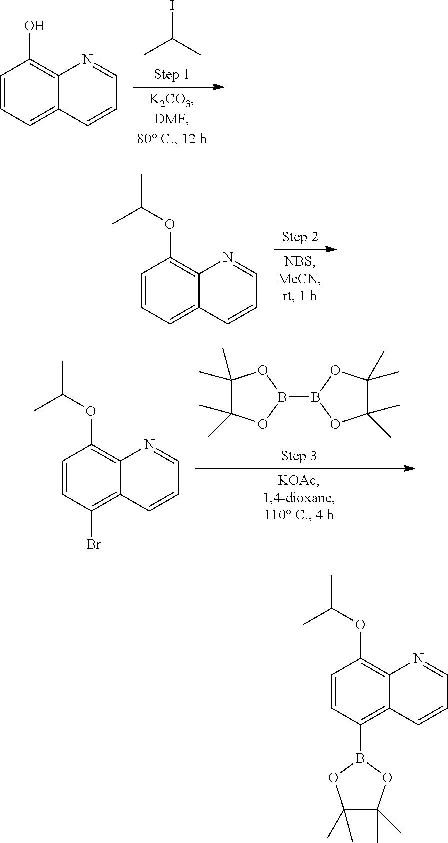

### Step 1: 8-isopropoxyquinoline

A solution of quinolin-8-ol (250 mg, 1.72 mmol) in DMF (8.6 mL) was treated with K₂CO₃ (0.6 g, 4.31 mmol) and 2-iodopropane (0.49 mL, 4.31 mmol). The reaction mixture was stirred at 80° C. for 12 h. After cooling, the mixture was diluted with EtOAc and washed with NaHCO₃ sat. sol., brine, dried over Na₂SO₄, filtered, and concentrated in vacuo. The crude product was purified by flash chromatography (0-100% EtOAc in petroleum ether) to afford the title compound as an orange oil (168 mg, 52%). ¹H NMR (400 MHz, DMSO-d₆) δ 8.84 (dd, J=4.2, 1.8 Hz, 1H), 8.29 (dd, J=8.3, 1.8 Hz, 1H), 7.53-7.48 (m, 3H), 7.23-7.20 (m, 1H), 4.91-4.82 (m, 1H), 1.37 (d, J=6.1 Hz, 6H); LCMS (ES⁺) Method 2: m/z 188 (M+H)⁺, RT 0.81 min.

### Step 2: 5-bromo-8-isopropoxyquinoline

A solution of 8-isopropoxyquinoline (168 mg, 0.90 mmol) in MeCN (5.9 mL) was treated with 1-bromopyrrolidine-2,5-dione (0.16 g, 0.90 mmol) and stirred at rt 1 h. The reaction mixture was diluted with EtOAc, washed with H₂O, dried over Na₂SO₄, filtered, and concentrated in vacuo to afford the title compound as an orange powder (238 mg, 99%). ¹H NMR (400 MHz, DMSO-d₆) δ 8.92 (dd, J=4.1, 1.6 Hz, 1H), 8.42 (dd, J=8.6, 1.5 Hz, 1H), 7.83 (d, J=8.6 Hz, 1H), 7.70 (dd, J=8.6, 4.2 Hz, 1H), 7.19 (d, J=8.3 Hz, 1H), 4.91-4.82 (m, 1H), 1.37 (d, J=5.9 Hz, 6H); LCMS (ES⁺) Method 2: m/z 265, 267 (M+H)⁺, RT 1.72 min.

### Step 3: 8-isopropoxy-5-(4,4,5,5-tetramethyl-1,3,2-dioxaborolan-2-yl)quinoline

The title compound was synthesized using the procedure reported for Intermediate 68 step 2 and obtained as a brown oil. LCMS (ES⁺) Method 2: m/z 314 (M+H)⁺, RT 1.73 min.

### Intermediate 73: 8-cyclopropyl-5-(4,4,5,5-tetramethyl-1,3,2-dioxaborolan-2-yl)quinoline

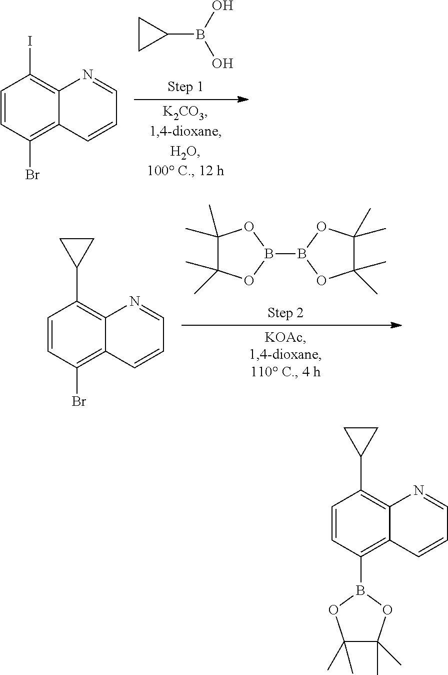

### Step 1: 5-bromo-8-cyclopropylquinoline A suspension of cyclopropylboronic acid (12.4 mg, 0.14 mmol), 5-bromo-8-iodoquinoline (40 mg, 0.12 mmol), Pd(dppf)Cl₂·DCM (19.8 mg, 0.02 mmol) and K₂CO₃ (57.9 mg, 0.42 mmol) in 1,4-dioxane (0.54 mL) and H₂O (0.054 mL) was stirred at 100° C. for 12 h. After cooling the reaction mixture was filtered on a pad of solka-floc which was washed with EtOAc. The organic phase was washed with H₂O, brine, dried over Na₂SO₄, filtered, and concentrated in vacuo. The crude product was purified by flash chromatography (0-100% EtOAc in petroleum ether) to afford the title compound (25 mg, 84%). ¹H NMR (400 MHz, CDCl₃) δ 9.00 (br d, J=2.4 Hz, 1H), 8.55 (br d, J=8.3 Hz, 1H), 7.72 (d, J=7.9 Hz, 1H), 7.53 (br dd, J=8.6, 4.2 Hz, 1H), 7.05 (d, J=7.9 Hz, 1H), 3.20-3.13 (m, 1H), 1.29-1.19 (m, 2H), 0.90-0.83 (m, 2H); LCMS (ES⁺) Method 2: m/z 248-250 (M+H)⁺, RT 2.12 min.

### Step 2: 8-cyclopropyl-5-(4,4,5,5-tetramethyl-1,3,2-dioxaborolan-2-yl)quinoline

The title compound was synthesized using the procedure reported for Intermediate 15 and obtained as a brown oil. LCMS (ES⁺) Method 2: m/z 296 (M+H)⁺, RT 1.92 min.

### Intermediate 75: 8-chloro-7-((5-chloropyrazin-2-yl)thio)-N,N-dimethylimidazo[1,2-a]pyridine-2-carboxamide

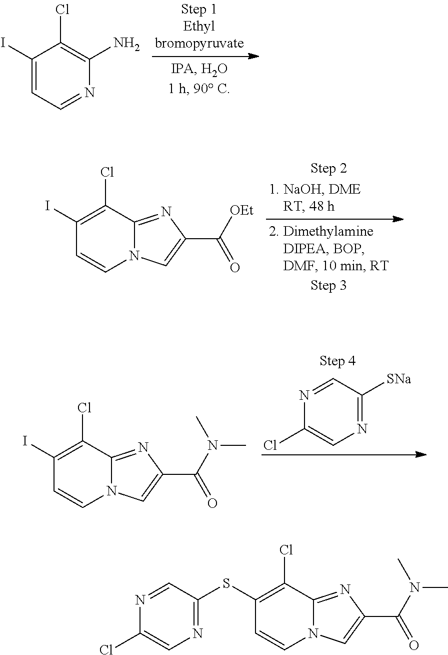

### Step 1: ethyl 8-chloro-7-iodoimidazo[1,2-a]pyridine-2-carboxylate

Ethyl bromopyruvate (0.2 mL, 1.6 mmol) was added to a solution of 3-chloranyl-4-iodanyl-pyridin-2-amine (200 mg, 0.79 mmol) in IPA (1 mL) and H₂O (1 mL). The mixture was heated for 1 h at 90° C. under MW irradiation, then was filtered and the pale-yellow powder was washed with Et₂O and dried under reduced pressure to afford the title compound (167 mg, 61%). LCMS (ES⁺) Method 1A: m/z 351, RT 1.66 min.

### Step 2: 8-chloro-7-iodoimidazo[1,2-a]pyridine-2-carboxylic acid

5N NaOH aq. (2 mL, 10 mmol) was added to a suspension of ethyl 8-chloro-7-iodoimidazo[1,2-a]pyridine-2-carboxylate (167 mg, 0.48 mmol) in DME (1.5 mL) and the reaction mixture was stirred at rt for 48 h. The mixture was filtered, the obtained white solid was washed with DME, and cold H₂O then was dried under reduced pressure to afford the title compound (92 mg, 60%). LCMS (ES⁺) Method 1A: m/z 323, RT 1.18 min.

### Step 3: 8-chloro-7-iodo-N,N-dimethylimidazo[1,2-a]pyridine-2-carboxamide

Dry DIPEA (0.18 mL, 1.02 mmol) and BOP (126 mg, 0.28 mmol) were added to a solution of 8-chloro-7-iodoimidazo[1,2-a]pyridine-2-carboxylic acid (83 mg, 0.26 mmol) and dimethylamine hydrochloride (32 mg, 0.39 mmol) in dry DMF (1 mL). The resulting solution was stirred at rt for 10 min then was directly purified by reverse phase chromatography (eluting with 0%-30% H₂O/CH₃CN+0.1% TFA). Fractions containing the desired product were collected, basified with NaHCO₃ powder and extracted with EtOAc to afford the title compound as white solid (62 mg, 90%). LCMS (ES⁺) Method 1A: m/z 350, RT 1.38 min.

### Step 4: 8-chloro-7-((5-chloropyrazin-2-yl)thio)-N,N-dimethylimidazo[1,2-a]pyridine-2-carboxamide

Dry DIPEA (0.05 mL, 0.29 mmol) was added to a mixture of 8-chloro-7-iodo-N,N-dimethylimidazo[1,2-a]pyridine-2-carboxamide (50 mg, 0.14 mmol), sodium 5-chloropyrazine-2-thiolate (prepared as reported for the synthesis of Intermediate 98, steps 2-3, starting from 2-bromo-5-chloropyrazine) (31 mg, 0.15 mmol), XantPhos (8.3 mg, 0.014 mmol) and Pd₂(dba)₃ (6.5 mg, 0.007 mmol) in dry 1,4-dioxane (0.7 mL). The reaction mixture was heated at 65° C. for 30 min, then was directly purified by flash chromatography on silica gel (from 0-100% EtOAc in DCM) to obtain the title compound as light-brown solid (40 mg, 76%). ¹H NMR (300 MHz, DMSO-d₆) δ 8.72-8.67 (m, 1H), 8.63-8.52 (m, 2H), 8.45 (s, 1H), 7.12 (d, J=7.2 Hz, 1H), 3.43 (s, 3H), 3.03 (s, 3H); LCMS (ES⁺) Method 1A: m/z 368, RT 1.58 min.

### Intermediate 80: 8-ethoxy-5-(4,4,5,5-tetramethyl-1,3,2-dioxaborolan-2-yl)quinoline

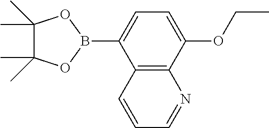 [SMILES: CCOc1ccc(B2OC(C)(C)C(C)(C)O2)c2cccnc12]

The title compound was synthesized using the procedure reported for Intermediate 72 using iodoethane in step 1 and obtained as an orange oil. LCMS (ES⁺) Method 2: m/z 174 (M+H)⁺, RT 0.63 min.

### Intermediate 81: 8-(1-methoxyethyl)-5-(4,4,5,5-tetramethyl-1,3,2-dioxaborolan-2-yl)quinoline

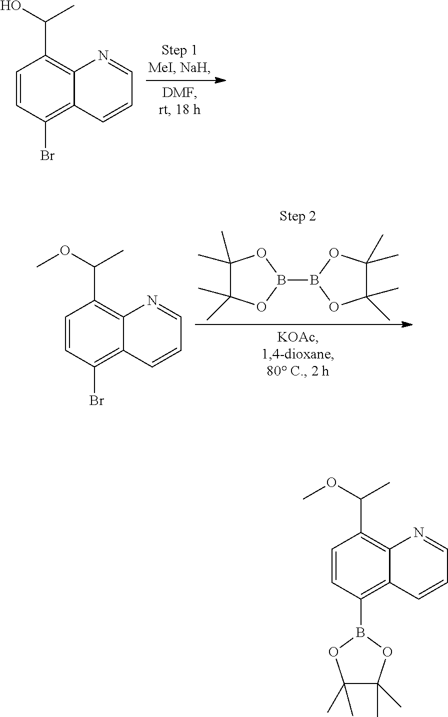

### Step 1: 5-bromo-8-(I-methoxyethyl)quinoline

A solution of 1-(5-bromoquinolin-8-yl)ethan-1-ol (prepared as reported in the synthesis of Intermediate 69; 210 mg, 0.83 mmol) in dry DMF (4.0 mL) was treated with NaH (60% in mineral oil; 30 mg, 1.25 mmol) and Mel (0.08 mL, 1.25 mmol). The resulting mixture was stirred at rt for 18 h. NH₄Cl sat. sol. was added, and the reaction mixture was stirred for 10 min before being diluted with EtOAc and washed with brine. The organic phase was dried over MgSO₄, filtered, and concentrated in vacuo. The crude product was purified by flash chromatography (0-60% EtOAc in petroleum ether) to afford the title compound as an orange oil (165 mg, 74%). LCMS (ES⁺) Method 2: m/z 266, 268 (M+H)⁺, RT 2.30 min.

### Step 2: 8-(1-methoxyethyl)-5-(4,4,5,5-tetramethyl-1,3,2-dioxaborolan-2-yl)quinoline

The title compound was synthesized using the procedure reported for Intermediate 68 step 2 and obtained as a white powder. LCMS (ES⁺) Method 2: m/z 314 (M+H)⁺, RT 2.20 min.

### Intermediate 94: benzyl (((1R,5S,6r)-6-(5-methylisoxazol-3-yl)-3-azabicyclo[3.1.0]hexan-6-yl)methyl)carbamate hydrochloride

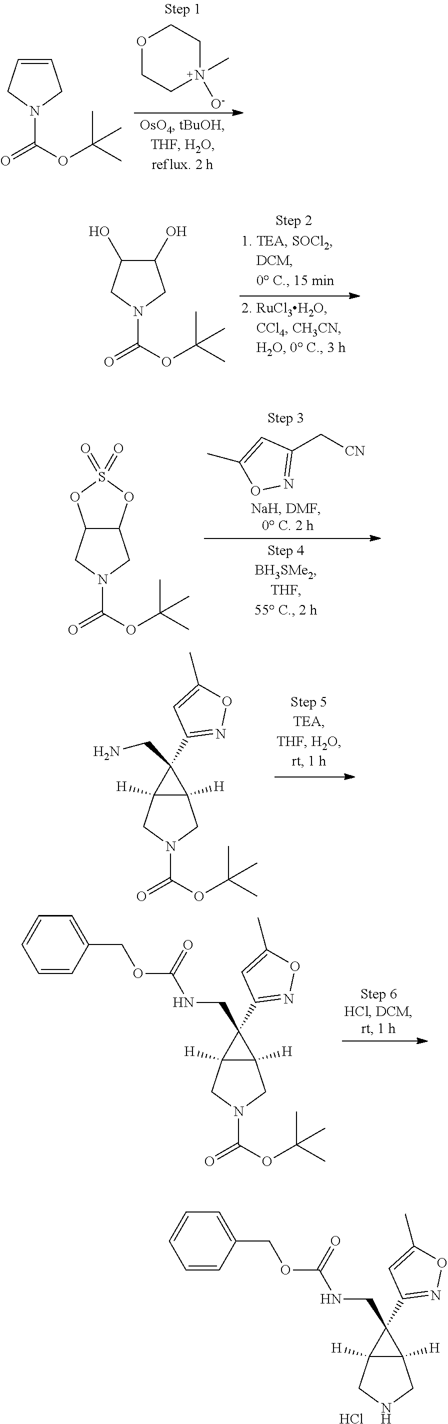

### Step 1: tert-butyl 3,4-dihydroxypyrrolidine-1-carboxylate

The title compound was prepared following procedure reported in the synthesis of Intermediate 1 in step 1 and obtained as a colourless oil (1.03 g, 78%). ¹H NMR (400 MHz, CDCl₃) δ 4.28-4.24 (m, 2H), 3.62-3.58 (br m, 2H), 3.40-3.32 (br m, 2H), 2.50-2.41 (br m, 2H), 1.46 (s, 9H). LCMS (ES⁺) Method 2: RT 0.88 min.

### Step 2: tert-butyl tetrahydro-5H-[1,3,2]dioxathiolo[4,5-c]pyrrole-5-carboxylate 2,2-dioxide

The title compound was prepared following procedure reported in the synthesis of Intermediate 1 in step 2 and obtained as a colourless oil (1.25 g, 96%). ¹H NMR (400 MHz, CDCl₃) δ 5.37-5.34 (m, 2H), 4.14-3.99 (br m, 2H), 3.67-3.60 (br m, 2H), 1.48 (s, 9H). LCMS (ES⁺) Method 2: RT 1.51 min.

### Step 3: tert-butyl (1R,5S,6s)-6-cyano-6-(5-methylisoxazol-3-yl)-3-azabicyclo[3.1.0]hexane-3-carboxylate

The title compound was prepared following the procedure reported in the synthesis of Intermediate 6 step 1 and obtained as a yellow powder (190 mg, 58%). ¹H NMR (400 MHz, CDCl₃) δ 6.20 (s, 1H), 3.99 (d, J=12.1 Hz, 1H), 3.90 (d, J=12.3 Hz, 1H), 3.74-3.69 (m, 2H), 2.64-2.59 (m, 2H), 2.43 (s, 3H), 1.48 (s, 9H). LCMS (ES⁺) Method 2: m/z 312 (M+Na)⁺, RT 1.61 min.

### Step 4: tert-butyl (1R,5S,6r)-6-(aminomethyl)-6-(5-methylisoxazol-3-yl)-3-azabicyclo[3.1.0]hexane-3-carboxylate

The title compound was prepared following the procedure reported in the synthesis of Intermediate 6 step 2 and used as a crude. LCMS (ES⁺) Method 2: m/z 294 (M+H)⁺, RT 1.02 min.

### Step 5: tert-butyl (1R,5S,6r)-6-((((benzyloxy)carbonyl)amino)methyl)-6-(5-methylisoxazol-3-yl)-3-azabicyclo[3.1.0]hexane-3-carboxylate

The title compound was prepared following the procedure reported in the synthesis of Intermediate 6 step 3 (benzyl (2,5-dioxopyrrolidin-1-yl) carbonate in a mixture of THF and H₂O was used) and obtained as a colourless oil (150 mg, 51%). LCMS (ES⁺) Method 2: m/z 450 (M+Na)⁺, RT 1.98 min.

### Step 6: benzyl (((1R,5S,6r)-6-(5-methylisoxazol-3-yl)-3-azabicyclo[3.1.0]hexan-6-yl)methyl)carbamate hydrochloride

The title compound was prepared following the procedure reported in the synthesis of Intermediate 6 step 4 and obtained as a white powder. LCMS (ES⁺) Method 2: m/z 328 (M+H)⁺, RT 1.12 min.

The following compounds were synthesized according to the procedure of Intermediate 94 in step 3 using the appropriate nitriles.

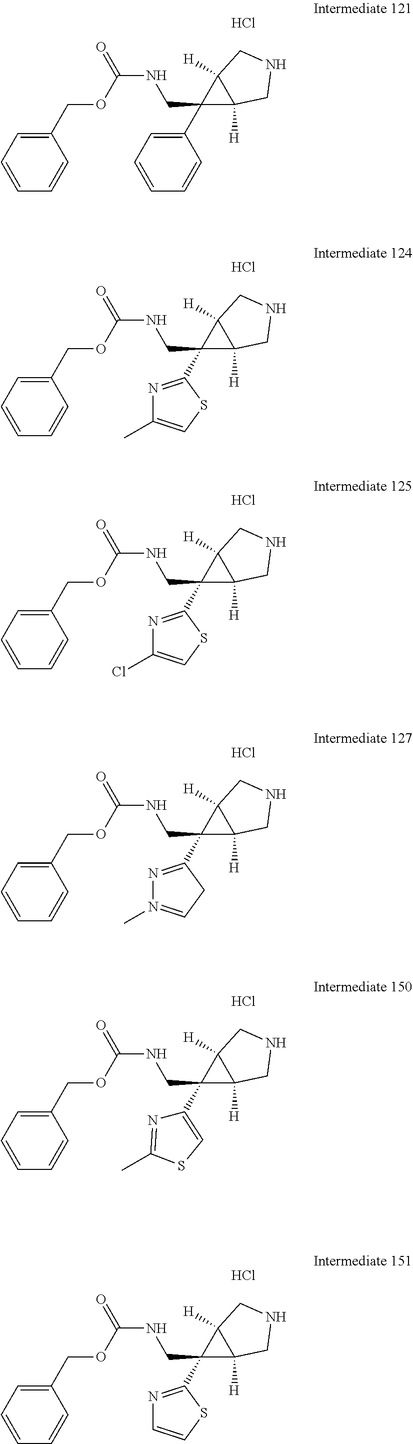

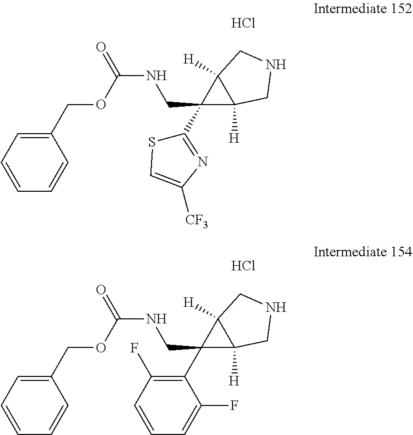 [SMILES: Cl ; O=C(NC[C@]1(c2nc(C(F)(F)F)cs2)[C@@H]2CNC[C@@H]21)OCc1ccccc1 ; Cl ; O=C(NC[C@]1(c2c(F)cccc2F)[C@@H]2CNC[C@@H]21)OCc1ccccc1]

### Intermediate 95: ethyl 7-(((tert-butoxycarbonyl)amino)methyl)-3-azabicyclo[4.1.0]heptane-7-carboxylate

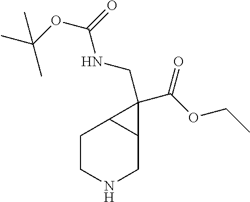 [SMILES: CCOC(=O)C1(CNC(=O)OC(C)(C)C)C2CCNCC21]

The title compound was synthesized using the procedure reported for Intermediate 9 using Intermediate 8 and ethyl 2-cyanoacetate in step 1; NaBH₄ and CoCl₂ in EtOH were used in step 2. The title compound was obtained as a colorless oil. ¹H NMR (400 MHz, CDCl₃) δ 5.28 (br s, 1H), 4.15-4.10 (m, 2H), 3.88-3.69 (m, 3H), 3.34-3.28 (m, 1H), 3.11 (br d, J=13.8 Hz, 1H), 2.93-2.85 (m, 1H), 2.63-2.56 (m, 1H), 2.03-1.88 (m, 2H), 1.73-1.61 (m, 2H), 1.44 (s, 9H), 1.27-1.23 (m, 3H); LCMS (ES⁺) Method 2: m/z 299 (M+H)⁺, RT 0.96 min.

### Intermediate 96: 5-bromo-2-chloro-3-methyl-7-((2-(trimethylsilyl)ethoxy)methyl)-3,7-dihydro-4H-pyrrolo[2,3-d]pyrimidin-4-one

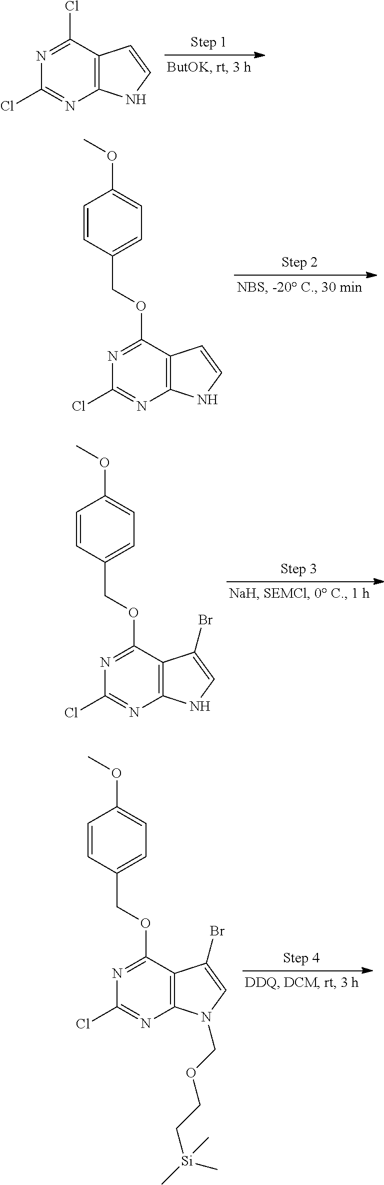

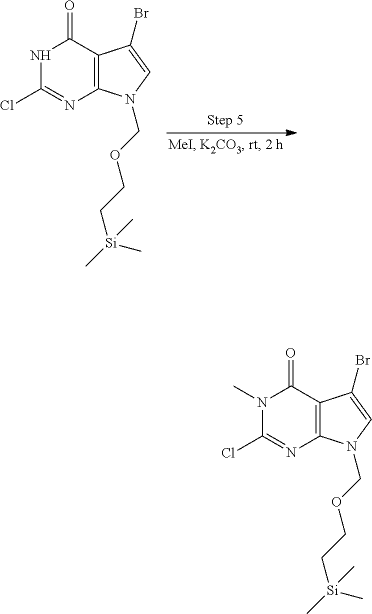 [SMILES: C[Si](C)(C)CCOCn1cc(Br)c2c(=O)[nH]c(Cl)nc21 ; Cn1c(Cl)nc2c(c(Br)cn2COCC[Si](C)(C)C)c1=O]

### Step 1: 2-chloro-4-((4-methoxybenzyl)oxy)-7H-pyrrolo[2,3-d]pyrimidine

2,4-bis(chloranyl)-7H-pyrrolo[2,3-d]pyrimidine (2.5 g, 13.3 mmol) and (4-methoxyphenyl)methanol (2 mL, 16 mmol) were dissolved in 1,4-dioxane (38 mL) and the obtained mixture was treated with tBuOK (6 g, 53.2 mmol) and stirred at rt for 3 h. The reaction was quenched by addition of NH₄Cl sat. sol. then the precipitate was collected and washed with H₂O, EtOAc and dried to give the desired product as white solid. The organic phase coming from the filtrate solution was isolated, dried over Na₂SO₄, filtered and concentrated. The crude product was then triturated with DCM to give additional final compound. Both powders were put together giving the desired product as white solid (3.46 g, 90%). LCMS (ES⁺) Method 2: m/z 288, 290 (M+H)⁺, RT 1.91 min.

### Step 2: 5-bromo-2-chloro-4-((4-methoxybenzyl)oxy)-7H-pyrrolo[2,3-d]pyrimidine

5-bromanyl-2-chloranyl-4-[(4-methoxyphenyl)methoxy]-7H-pyrrolo[2,3-d]pyrimidine (4.4 g, 11.9 mmol) was dissolved in DMF (35 mL) and the obtained solution was cooled to −20° C. and treated with 1-bromopyrrolidine-2,5-dione (2.34 g, 13.1 mmol). The reaction mixture was stirred at this temperature for 30 min then quenched by addition of 1M Na₂S₂O₃ aq. sol. (30 mL) and EtOAc (15 mL) and stirred at rt for additional 30 min. The precipitate was filtered off, washed with H₂O, and dried giving the title product as beige solid (4.4 g, 99.9%). LCMS (ES⁺) Method 2: m/z 367, 369 (M+H)⁺, RT 2.1 min

### Step 3: 5-bromo-2-chloro-4-((4-methoxybenzyl)oxy)-7-((2-(trimethylsilyl)ethoxy)methyl)-7H-pyrrolo[2,3-d]pyrimidine

5-bromanyl-2-chloranyl-4-[(4-methoxyphenyl)methoxy]-7H-pyrrolo[2,3-d]pyrimidine (4.4 g, 11.9 mmol) was dissolved in DMF (91.8 mL) and the obtained solution was cooled to 0° C. and sequentially treated with NaH (60% in mineral oil; 0.57 g, 14.32 mmol) and SEM-C₁ (2.54 mL, 14.32 mmol). The reaction mixture was stirred at 0° C. for 1 h then quenched by addition of NH₄Cl sat. aq. sol. EtOAc was added, and the organic phase was isolated and washed with H₂O, brine, dried over Na₂SO₄, filtered, and concentrated in vacuo. The crude product was purified by flash chromatography (0-50% EtOAc in petroleum ether) to afford the title compound as white solid (4.5 g, 75%). LCMS (ES⁺) Method 2: m/z 498, 500 (M+H)⁺, RT 2.83 min.

### Step 4: 5-bromo-2-chloro-7-((2-(trimethylsilyl)ethoxy)methyl)-3,7-dihydro-4H-pyrrolo[2,3-d]pyrimidin-4-one

2-[[5-bromanyl-2-chloranyl-4-[(4-methoxyphenyl)methoxy]pyrrolo[2,3-d]pyrimidin-7-yl]methoxy]ethyl-trimethyl-silane (2 g, 4.01 mmol) was dissolved in DCM (37.6 mL) and treated with 4,5-dichloro-3,6-dioxo-cyclohexa-1,4-diene-1,2-dicarbonitrile (2.73 g, 12.03 mmol) and H₂O (2 mL). The reaction mixture was stirred at rt for 3 days then quenched by addition of NaHCO₃ sat. aq. sol. and DCM. The mixture was then filtered on a pad of cellulose washing with DCM and H₂O. The filtrate was extracted with DCM, dried over Na₂SO₄, filtered and concentrated. The crude product was purified by flash chromatography (0-50% EtOAc in petroleum ether) to afford the title compound as pale-yellow solid (1.1 g, 72%). LCMS (ES⁺) Method 2: m/z 378, 380 (M+H)⁺, RT 2.08 min.

### Step 5: 5-bromo-2-chloro-3-methyl-7-((2-(trimethylsilyl)ethoxy)methyl)-3,7-dihydro-4H-pyrrolo[2,3-d]pyrimidin-4-one

5-bromanyl-2-chloranyl-7-(2-trimethylsilylethoxymethyl)-3H-pyrrolo[2,3-d]pyrimidin-4-one (1.1 g, 2.9 mmol) was dissolved in DMF (11 mL) and treated with K₂CO₃ (0.8 g, 5.76 mmol) and Mel (0.36 mL, 5.76 mmol). The reaction mixture was stirred at rt for 2 h then poured into H₂O and extracted with EtOAc. The organic phase was washed with H₂O, brine, dried over Na₂SO₄ filtered and concentrated in vacuo. The crude product was purified by flash chromatography (0-50% EtOAc in petroleum ether) to afford the title compound as white solid (0.96 g, 85%). ¹H NMR (400 MHz, DMSO-d₆) δ 7.49 (s, 1H), 5.39 (s, 2H), 3.57 (s, 3H), 3.52 (t, J=7.9 Hz, 2H), 0.84 (t, J=8.3 Hz, 2H), 0 (s, 9H). LCMS (ES⁺) Method 2: m/z 392, 394, 396 (M+H)⁺, RT 2.36 min.

### Intermediate 97: 6-chloropyrido[2,3-b]pyrazin-2-yl 4-nitrobenzenesulfonate

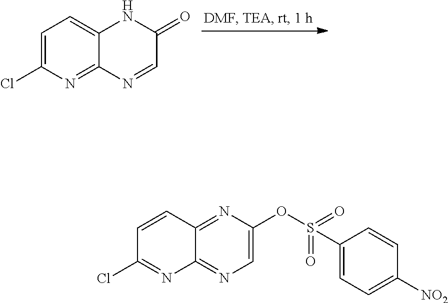 [SMILES: O=c1cnc2nc(Cl)ccc2[nH]1 ; O=[N+]([O-])c1ccc(S(=O)(=O)Oc2cnc3nc(Cl)ccc3n2)cc1]

6-chloropyrido[2,3-b]pyrazin-2(1H)-one (1 g, 5.5 mmol) was dissolved in DMF (10 mL) then TEA (0.92 mL, 6.6 mmol) and 4-nitrobenzenesulfonyl chloride (1.22 g, 5.51 mmol) were added. The mixture was stirred at rt for 1 h then poured into H₂O and the formed precipitate was filtered off and dried to afford the title product as brown solid (1.87 g, 92.5%). ¹H NMR (400 MHz, DMSO-d₆) δ 9.13 (s, 1H), 8.52-5.50 (m, 5H), 8.01 (d, J=8.7 Hz, 1H). LCMS (ES⁺) Method 2: m/z 367 (M+H)⁺, RT 1.78 min.

### Intermediate 98: sodium 3-chloro-2-((4-methoxybenzyl)amino)pyridine-4-thiolate

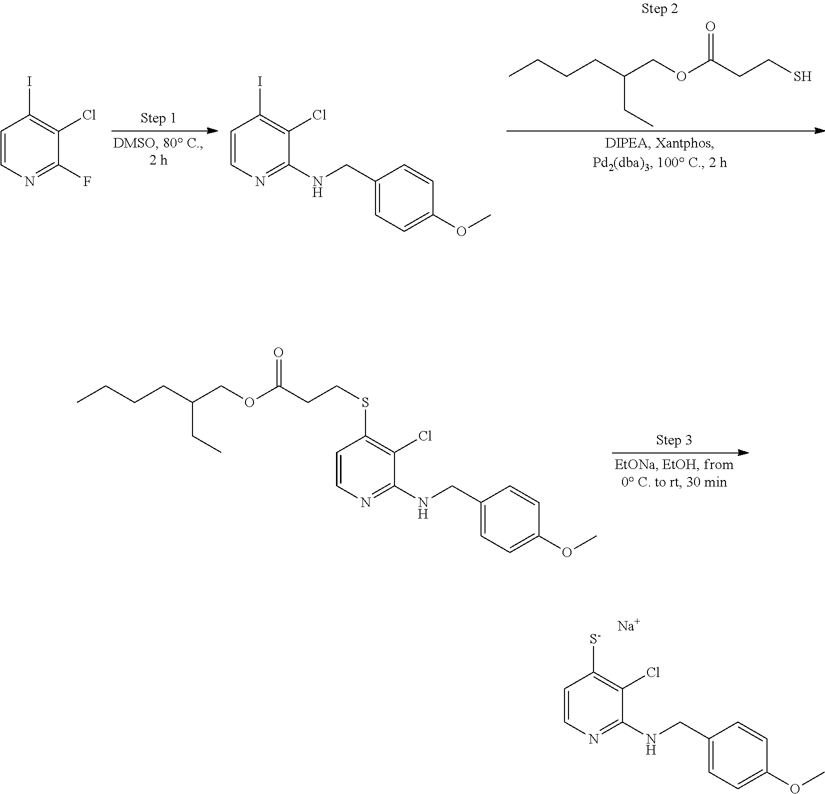 [SMILES: Fc1nccc(I)c1Cl ; COc1ccc(CNc2nccc(I)c2Cl)cc1 ; CCCCC(CC)COC(=O)CCS ; CCCCC(CC)COC(=O)CCSc1ccnc(NCc2ccc(OC)cc2)c1Cl ; [Na+] ; COc1ccc(CNc2nccc([S-])c2Cl)cc1]

### Step 1: 3-chloro-4-iodo-N-(4-methoxybenzyl)pyridin-2-amine

3-chloranyl-2-fluoranyl-4-iodanyl-pyridine (5 g, 19.4 mmol) and (4-methoxyphenyl)methanamine (5.1 mL, 38.9 mmol) were dissolved in DMSO (39 mL) and the obtained solution was stirred at 80° C. for 2 h. After completion, EtOAc was added and the organic solution was washed with H₂O (2×), brine (2×), dried over Na₂SO₄, filtered, and concentrated in vacuo. The crude product was purified by flash chromatography (0-50% EtOAc in petroleum ether) to afford the title compound as colourless oil (7 g, 98%). LCMS (ES⁺) Method 2: m/z 375 (M+H)⁺, RT 2.31 min.

### Step 2: 2-ethylhexyl 3-((3-chloro-2-((4-methoxybenzyl)amino)pyridin-4-yl)thio)propanoate

The title compound was prepared according to the procedure reported for Intermediate 41 step 1 and obtained as orange oil (6 g, 99%). LCMS (ES⁺) Method 2: m/z 465, 467 (M+H)⁺, RT 2.84 min.

### Step 3: sodium 3-chloro-2-((4-methoxybenzyl)amino)pyridine-4-thiolate

2-ethylhexyl 3-((3-chloro-2-((4-methoxybenzyl)amino)pyridin-4-yl)thio)propanoate (1 g, 2.27 mmol) was dissolved in MeOH (23 mL) and NaOMe (2.1 mL, 2.3 mmol) was added. The mixture was stirred at rt for 2 h then the solvent was concentrated in vacuo. The obtained residue was dissolved with H₂O, washed with Et₂O (3×) then the aqueous phase was diluted with MeCN and lyophilized to afford the title product as pale-yellow solid (0.76 g, 97%). ¹H NMR (400 MHz, D₂O) δ 8.34 (d, J=5.2 Hz, 1H), 8.24 (d, J=8.8 Hz, 2H), 7.87 (d, J=8.8 Hz, 2H), 7.77 (d, J=5.7 Hz, 1H), 4.42 (s, 2H), 3.74 (s, 3H). LCMS (ES⁺) Method 2: m/z 281, 283 (M+H)⁺, RT 1.36 min.

### Intermediate 99: 6-bromo-1,5-naphthyridin-2-yl 4-nitrobenzenesulfonate

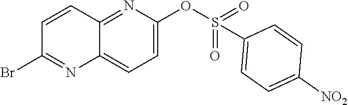 [SMILES: O=[N+]([O-])c1ccc(S(=O)(=O)Oc2ccc3nc(Br)ccc3n2)cc1]

The above intermediate was synthesized using the procedure reported for Intermediate 97 using 6-bromo-1,5-naphthyridin-2(1H)-one and obtained as a brown solid (246 mg, 78%). LCMS (ES⁺) Method 2: m/z 410, 412 (M+H)⁺, RT 1.99 min.

### Intermediate 100 & 101: sodium 3-chloro-2-(1H-pyrazol-1-yl)pyridine-4-thiolate & 2-chloro-5-((3-chloro-2-(1H-pyrazol-1-yl)pyridin-4-yl)thio)pyrazine

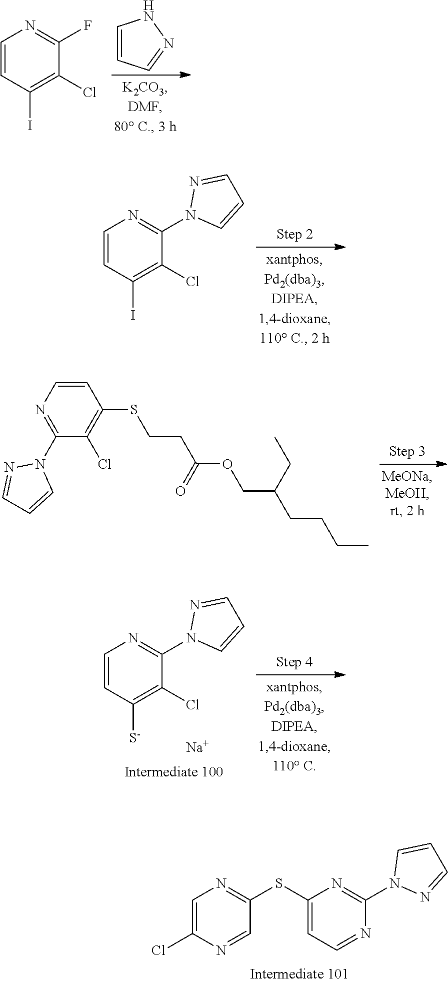

### Step 1: 3-chloro-4-iodo-2-(1H-pyrazol-1-yl)pyridine

A solution of 3-chloro-2-fluoro-4-iodopyridine (200 mg, 0.78 mmol), 1H-pyrazole (105.8 mg, 1.55 mmol) and K₂CO₃ (217.9 mg, 1.55 mmol) in DMF (3 mL) was stirred at 80° C. for 3 h. After cooling the mixture was treated with 5% citric acid and the resulting aqueous layer was extracted with DCM (3×2 mL). The collected organic layers were washed with brine, dried over Na₂SO₄, filtered, and concentrated in vacuo to give a residue which was purified by flash chromatography (0-50% EtOAc in petroleum ether) to afford the title compound as an amorphous white solid (196 mg, 83%). LCMS (ES⁺) Method 2 m/z 306 (M+H)⁺, RT 1.70 min.

### Step 2: 2-ethylhexyl 3-((3-chloro-2-(1H-pyrazol-1-yl)pyridin-4-yl)thio)propanoate

3-chloranyl-4-iodanyl-2-pyrazol-1-yl-pyridine (500 mg, 1.64 mmol), 2-ethylhexyl 3-sulfanylpropanoate (0.4 mL, 1.8 mmol), Pd₂(dba)₃ (40 mg, 0.04 mmol), XantPhos (51 mg, 0.09 mmol) were dissolved in 1,4-dioxane (8 mL) then DIPEA (0.57 mL, 3.27 mmol) was added, and the mixture was heated at 100° C. for 2 h. The reaction mixture was filtered on a pad of cellulose then the solvent was concentrated in vacuo. The crude product was purified by flash chromatography (0-100% EtOAc in petroleum ether) to afford the title compound as orange oil (640 mg, 99%). LCMS (ES⁺) Method 2 m/z 396, 398 (M+H)⁺, RT 2.49 min.

### Step 3: sodium 3-chloro-2-(1H-pyrazol-1-yl)pyridine-4-thiolate (Intermediate 100)

sodium 3-chloro-2-(1H-pyrazol-1-yl)pyridine-4-thiolate was prepared according to the synthesis of Intermediate 98 step 3. Intermediate 100 was obtained as a yellow solid (150 mg, 100%). LCMS (ES⁺) Method 2 m/z 212, 214 (M+H)⁺, RT 1.17 min.

### Step 4: 2-chloro-5-((3-chloro-2-(1H-pyrazol-1-yl)pyridin-4-yl)thio)pyrazine (Intermediate 101)

Intermediate 100 (73 mg, 0.31 mmol), 2-chloro-5-iodopyrazine (50 mg, 0.21 mmol), XantPhos (6 mg, 0.01 mmol), Pd₂(dba)₃ (4.76 mg, 0.01 mmol) were dissolved in 1,4-dioxane (1 mL) then DIPEA (0.07 mL, 0.42 mmol) was added. The mixture was stirred at 100° C. for 2 h then diluted with EtOAc, filtered on a pad of cellulose and the solvent concentrated in vacuo. The crude product was purified by flash chromatography (0-100% EtOAc in petroleum ether) to afford the title compound as white oil (33 mg, 50%). ¹H NMR (400 MHz, DMSO-d₆) δ 8.91 (s, 1H), 8.84 (s, 1H), 8.34 (d, J=5.2 Hz, 1H), 8.28 (d, J=2.2 Hz, 1H), 7.83 (s, 1H), 7.38 (d, J=5.2 Hz, 1H), 6.57 (s, 1H). LCMS (ES⁺) Method 2: m/z 324, 326 (M+H)⁺, RT 1.77 min.

### Intermediate 103: ethyl (((1R,7S)-8-(2-fluorophenyl)-4-azabicyclo[5.1.0]octan-8-yl)methyl)carbamate hydrochloride

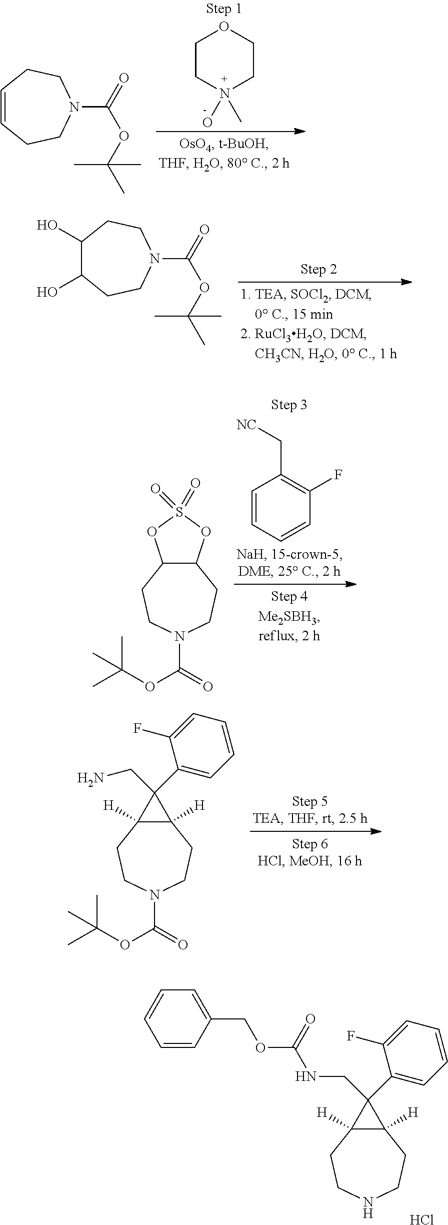

### Step 1: tert-butyl 4,5-dihydroxyazepane-1-carboxylate

The title compound was prepared following the procedure reported in the synthesis of Intermediate 1 step 1 and obtained as white solid (71 mg, 76%). LCMS (ES⁺) Method 2: RT 1.11 min.

### Step 2: tert-butyl hexahydro-6H-[1,3,2]dioxathiolo[4,5-d]azepine-6-carboxylate 2,2-dioxide

The title compound was prepared following the procedure reported in the synthesis of Intermediate 1 step 2 and obtained as pale-yellow oil (71 mg, 78%). ¹H NMR (400 MHz, CDCl₃) δ 5.18 (br s, 2H), 3.64 (br s, 2H), 3.47-3.41 (m, 2H), 3.34-2.28 (m, 2H), 2.11-2.08 (m, 2H), 1.45 (s, 9H).

### Step 3: tert-butyl (1R,7S)-8-cyano-8-(2-fluorophenyl)-4-azabicyclo[5.1.0]octane-4-carboxylate

The title compound was prepared following the procedure reported in the synthesis of Intermediate 39 step 2 using 2-(2-fluorophenyl)acetonitrile and obtained as yellow oil (60 mg, 75%). LCMS (ES⁺) Method 2: m/z 353 (M+Na)⁺, RT 2.04 min.

### Step 4: tert-butyl (1R,7S)-8-(aminomethyl)-8-(2-fluorophenyl)-4-azabicyclo[5.1.0]octane-4-carboxylate

The title compound was prepared following the procedure reported in the synthesis of Intermediate 39 step 3 and used as crude in the next step without further purification. LCMS (ES⁺) Method 2: m/z 336 (M+H)⁺, RT 1.48 min.

### Step 5: benzyl (1R,7S)-8-((((benzyloxy)carbonyl)amino)methyl)-8-(2-fluorophenyl)-4-azabicyclo[5.1.0]octane-4-carboxylate

The title compound was prepared following the procedure reported in the synthesis of Intermediate 28 step 3 using benzyl (2,5-dioxopyrrolidin-1-yl) carbonate in THF/H₂O and obtained as colourless oil (51 mg, 64%). LCMS (ES⁺) Method 2: m/z 491 (M+Na)⁺, RT 2.44 min.

### Step 6: benzyl (((1R,7S)-8-(2-fluorophenyl)-4-azabicyclo[5.1.0]octan-8-yl)methyl)carbamate hydrochloride

The title compound was prepared following the procedure reported in the synthesis of Intermediate 39 step 5 using a solution of HCl 1M in MeOH and obtained as colourless solid (44 mg, 99%). ¹H NMR (400 MHz, MeOH-d₄) δ 7.14-7.05 (m, 7H), 6.90-6.82 m, 2H), 4.79 (s, 2H), 3.40 (s, 2H), 3.35-3.31 (m, 2H), 2.98-2.92 (m, 2H), 2.31-2.27 (2H), 1.72-1.67 (m, 2H), 1.34-1.32 (m, 2H). LCMS (ES⁺) Method 2: m/z 369 (M+H)⁺, RT 1.46 min.

### Intermediate 104: sodium 3-fluoro-2-methylpyridine-4-thiolate

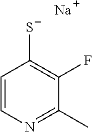

The above intermediate was synthesized as reported for Intermediate 98 step 2 & 3 starting from 4-bromo-3-fluoro-2-methylpyridine and obtained as a white solid. LCMS (ES⁺) Method 2: m/z 143 (M+H)⁺, RT 0.49 min.

### Intermediate 105: 7-chloro-6-(4,4,5,5-tetramethyl-1,3,2-dioxaborolan-2-yl)-3-((2-(trimethylsilyl)ethoxy)methyl)benzo[d]thiazol-2(3H)-one

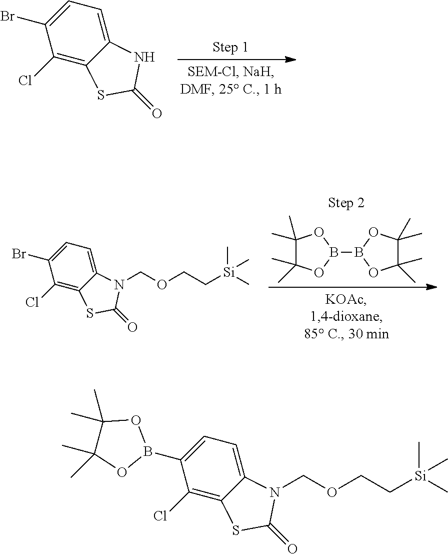

### Step 1: 6-bromo-7-chloro-3-((2-(trimethylsilyl)ethoxy)methyl)benzo[d]thiazol-2(3H)-one

6-bromo-7-chlorobenzo[d]thiazol-2(3H)-one (700 mg, 2.65 mmol) was dissolved in DMF (12 mL) and the obtained solution was cooled to 0° C. then NaH (60% in mineral oil; 138 mg, 3.44 mmol) was added. The mixture was stirred at this temperature for 30 min then SEM-C₁ (0.61 mL, 3.44 mmol) was added. The reaction was allowed to reach 25° C. and stirred for 1 h then NH₄Cl sat sol. was added. The mixture was washed with EtOAc (3×) and the combined organic layers were dried over Na₂SO₄, filtered and concentrated in vacuo. The crude product was purified by flash chromatography (0-100% EtOAc in petroleum ether) to afford the title compound as yellow solid (300 mg, 29%). ¹H NMR (400 MHz, CDCl₃) δ 7.59 (d, J=8.8 Hz, 1H), 7.07 (d, J=8.8 Hz, 1H), 5.34 (s, 2H), 3.62 (t, J=7.9 Hz, 2H), 0.92 (t, J=8.3 Hz, 2H), 0 (s, 9H).

### Step 2: 7-chloro-6-(4,4,5,5-tetramethyl-1,3,2-dioxaborolan-2-yl)-3-((2-(trimethylsilyl)ethoxy)methyl)benzo[d]thiazol-2(3H)-one

6-bromo-7-chloro-3-((2-(trimethylsilyl)ethoxy)methyl)benzo[d]thiazol-2(3H)-one (180 mg, 0.46 mmol) and B₂pin₂ (185 mg, 073 mmol) were reacted according to the procedure reported for Intermediate 11 step 2. The title product was obtained as a yellow solid (200 mg, 99%). ¹H NMR (400 MHz, CDCl₃) δ 7.75 (d, J=7.9 Hz, 1H), 7.13 (d, J=7.9 Hz, 1H), 5.35 (s, 2H), 3.61 (t, J=7.9 Hz, 2H), 1.37 (s, 12H), 0.92 (t, J=7.9 Hz, 2H), 0 (s, 9H). LCMS (ES⁺) Method 2: m/z 442 (M+H)⁺, RT 2.84 min.

### Intermediate 106: 4-chloro-2-(2-methoxyethyl)-5-(4,4,5,5-tetramethyl-1,3,2-dioxaborolan-2-yl)-2H-indazole

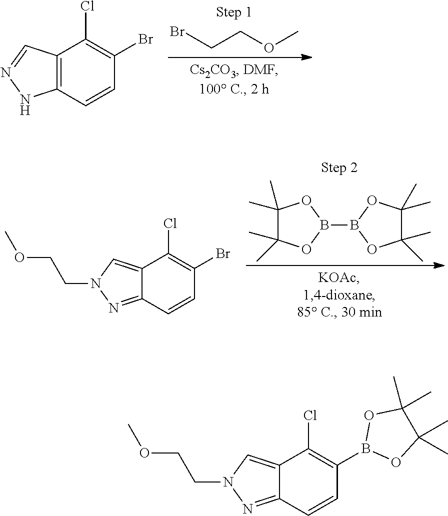

### Step 1: 5-bromo-4-chloro-2-(2-methoxyethyl)-2H-indazole

5-bromanyl-4-chloranyl-1H-indazole (200 mg, 0.86 mmol), Cs₂CO₃ (563 mg, 1.73 mmol) and 1-bromo-2-methoxyethane (121 μL, 1.3 mmol) were dissolved in DMF (6 mL) and the mixture was stirred for 3 h at 100° C. EtOAc and H₂O were added, and the organic solvent was isolated, washed with brine, dried over Na₂SO₄, filtered, and concentrated in vacuo. The crude product was purified by flash chromatography (0-50% EtOAc in petroleum ether) to afford the title compound as a pale-yellow solid (86 mg, 34%). ¹H NMR (400 MHz, CDCl₃) δ 8.07 (s, 1H), 7.49 (d, J=9.2 Hz, 1H), 7.43 (d, J=8.8 Hz, 1H), 4.58 (t, J=4.8 Hz, 2H), 3.88 (t, J=4.8 Hz, 2H), 3.36 (s, 3H). LCMS (ES⁺) Method 2: m/z 291 (M+H)⁺, RT 1.86 min.

### Step 2: 4-chloro-2-(2-methoxyethyl)-5-(4,4,5,5-tetramethyl-1,3,2-dioxaborolan-2-yl)-2H-indazole

5-bromo-4-chloro-2-(2-methoxyethyl)-2H-indazole (85 mg, 0.29 mmol) and B₂pin₂ (112 mg, 0.44 mmol) were reacted according to the procedure reported for Intermediate 11 step 2. After reaction completion, the mixture was filtered on a pad of cellulose then the solvent was concentrated, and the title product was used in the next step without further purification. LCMS (ES⁺) Method 2: m/z 337 (M+H)⁺, RT 2.03 min.

### Intermediate 107: 1-(difluoromethyl)-5-(4,4,5,5-tetramethyl-1,3,2-dioxaborolan-2-yl)-1H-benzo[d]imidazole & 1-(difluoromethyl)-6-(4,4,5,5-tetramethyl-1,3,2-dioxaborolan-2-yl)-1H-benzo[d]imidazole

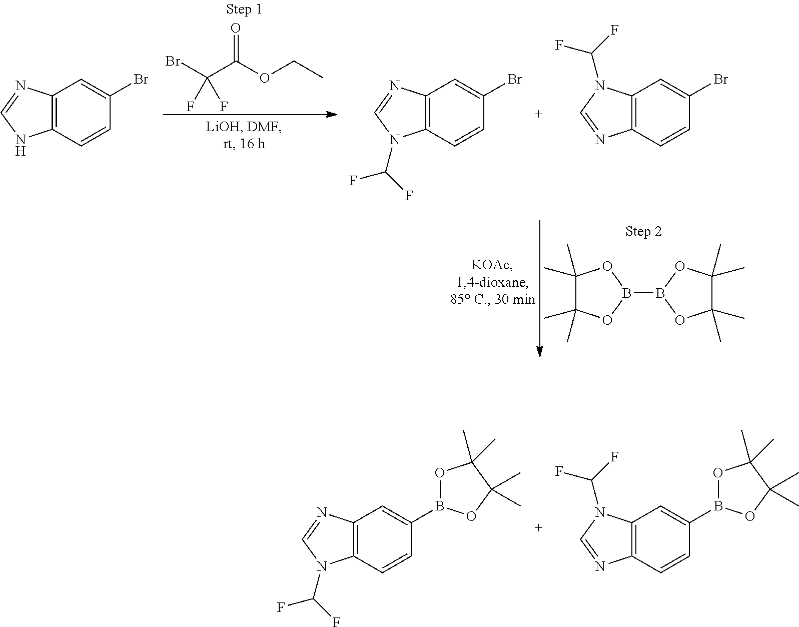

### Step 1: 5-bromo-1-(difluoromethyl)-1H-benzo[d]imidazole* & 6-bromo-1-(difluoromethyl)-1H-benzo[d]imidazole**

5-bromo-1H-benzo[d]imidazole (500 mg, 2.54 mmol) and ethyl 2-bromo-2,2-difluoroacetate (0.65 mL, 5.08 mmol) were dissolved in DMF (20 mL) then LiOH (243 mg, 10 mmol) was added and the mixture was stirred at rt for 16 h. EtOAc and brine were added, and the organic solvent was isolated, washed with brine, dried over Na₂SO₄, filtered and concentrated in vacuo The crude product was purified by flash chromatography (0-100% EtOAc in petroleum ether) to afford the title compound as a mixture of regioisomers (195 mg, 31%). LCMS (ES⁺) Method 2: m/z 246, 248 (M+H)⁺, RT 1.66 min. ¹H NMR (500 MHz, DMSO-d₆) δ 8.66 (s, 1H), 8.10 (t, J=59.1 Hz, 1H), 8.03* and 7.97** (d, J=1.9 Hz* and J=1.9 Hz**, 1H), 7.76** and 7.72* (d, J=9.1 Hz** and J=8.6 Hz*, 1H), 7.59-7.57* and 7.55-7.53** (m, 1H).

### Step 2: 1-(difluoromethyl)-5-(4,4,5,5-tetramethyl-1,3,2-dioxaborolan-2-yl)-1H-benzo[d]imidazole & 1-(difluoromethyl)-6-(4,4,5,5-tetramethyl-1,3,2-dioxaborolan-2-yl)-1H-benzo[d]imidazole

A mixture of 5-bromo-1-(difluoromethyl)-1H-benzo[d]imidazole & 6-bromo-1-(difluoromethyl)-1H-benzo[d]imidazole (195 mg, 0.79 mmol) and B₂pin₂ (300 mg, 1.18 mmol) were reacted according to the procedure reported for Intermediate 11 step 2. A mixture of title products was obtained and used in the next step without further purification. LCMS (ES⁺) Method 2: m/z 295 (M+H)⁺, RT 1.9 min.

### Intermediate 108: 2-chloro-5-((3-chloro-2-cyclopropylpyridin-4-yl)thio)pyrazine

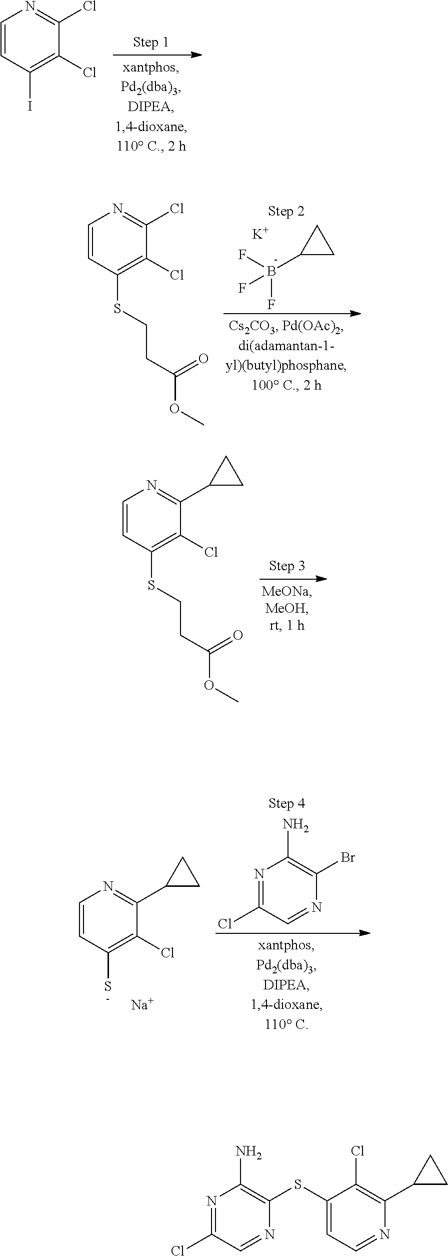

### Step 1: methyl 3-((2,3-dichloropyridin-4-yl)thio)propanoate

2,3-Dichloro-4-iodopyridine (1 g, 3.65 mmol) and methyl 3-mercaptopropanoate (482 mg, 4.0 mmol) were reacted according to the procedure reported for Intermediate 42 step 2 and obtained as yellow oil (950 mg, 98%). LCMS (ES⁺) Method 2: m/z 266, 268 (M+H)⁺, RT 1.85 min.

### Step 2: methyl 3-((3-chloro-2-cyclopropylpyridin-4-yl)thio)propanoate

Methyl 3-((2,3-dichloropyridin-4-yl)thio)propanoate (950 mg, 3.57 mmol), potassium cyclopropyltrifluoroborate (792 mg, 5.35 mmol), di(adamantan-1-yl)(butyl)phosphane (102 mg, 0.29 mmol) and Pd(OAc)₂ (40 mg, 0.18 mmol) were dissolved in a mixture of toluene (12 mL) and H₂O (1 mL). The obtained mixture was heated at 100° C. for 2 h then NH₄Cl sat. sol. (50 mL) was added, and the product was extracted with EtOAc. The combined organic layers were dried over Na₂SO₄, filtered, and concentrated in vacuo. The crude product was purified by flash chromatography (0-30% EtOAc in petroleum ether) to afford the title compound as yellow oil (80 mg, 8%). LCMS (ES⁺) Method 2: m/z 272, 274 (M+H)⁺, RT 1.94 min.

### Step 3: sodium 3-chloro-2-cyclopropylpyridine-4-thiolate

methyl 3-((3-chloro-2-cyclopropylpyridin-4-yl)thio)propanoate (80 mg, 0.29 mmol) was reacted according to the procedure reported for Intermediate 100 step 3. The title product was obtained as yellow solid (70 mg, 100%). LCMS (ES⁺) Method 2: m/z 186, 188 (M+H)⁺, RT 0.91 min.

### Step 4: 2-chloro-5-((3-chloro-2-cyclopropylpyridin-4-yl)thio)pyrazine

Sodium 3-chloro-2-cyclopropylpyridine-4-thiolate (15 mg, 0.072 mmol) and 3-bromo-6-chloropyrazin-2-amine (10 mg, 0.048 mmol) were reacted according to the procedure reported for Intermediate 101 step 4. The title product was obtained as yellow solid (5 mg, 33%). LCMS (ES⁺) Method 2: m/z 313, 315 (M+H)⁺, RT 2.02 min.

### Intermediate 109: benzyl (((1S,6R,7R)-7-(2-fluorophenyl)-3-(5-iodopyrazin-2-yl)-3-azabicyclo[4.1.0]heptan-7-yl)methyl)carbamate

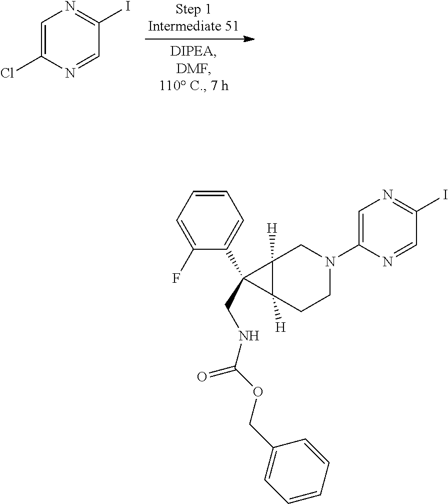 [SMILES: Clc1cnc(I)cn1 ; O=C(NC[C@]1(c2ccccc2F)[C@@H]2CCN(c3cnc(I)cn3)C[C@@H]21)OCc1ccccc1]

### Step 1: benzyl (((1S,6R,7R)-7-(2-fluorophenyl)-3-(5-iodopyrazin-2-yl)-3-azabicyclo[4.1.0]heptan-7-yl)methyl)carbamate

A solution of 2-chloro-5-iodopyrazine (25.0 mg, 0.07 mmol), Intermediate 51 (26.7 mg, 0.08 mmol) and DIPEA (71.7 uL, 0.41 mmol) in DMF (1 mL) was stirred for 7 h at 110° C. EtOAc was added, and the mixture was washed with brine, dried over Na₂SO₄, filtered and concentrated in vacuo. The crude product was purified by flash chromatography (0-20% EtOAc in petroleum ether) to afford the title compound as a yellow powder (17 mg, 44%). LCMS (ES⁺) Method 2: m/z 559 (M+H)⁺, RT 2.44 min.

### Intermediate 110: sodium 4-chloro-2-methyl-2H-indazole-5-thiolate

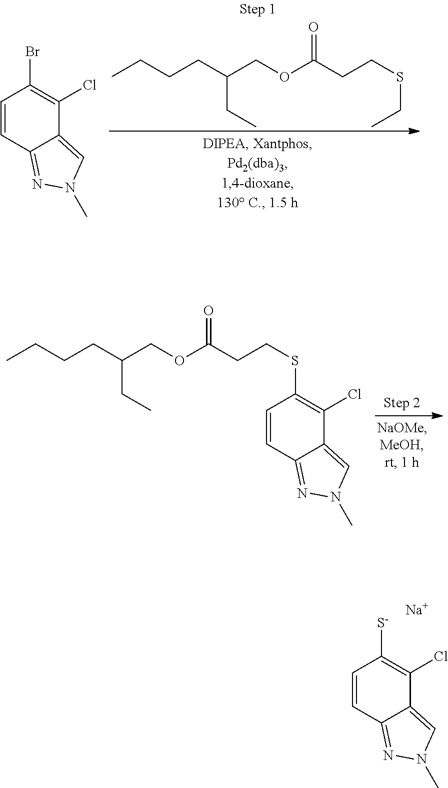

### Step 1: 2-ethylhexyl 3-((4-chloro-2-methyl-2H-indazol-5-yl)thio)propanoate

The title compound was prepared as reported in the synthesis of Intermediate 98 step 2 as a yellow oil (150 mg, 96%). LCMS (ES⁺) Method 2: m/z 383 (M+H)⁺, RT 2.58 min.

### Step 2: sodium 4-chloro-2-methyl-2H-indazole-5-thiolate

The title compound was prepared as reported in the synthesis of Intermediate 98 step 3 as a yellow solid (72 mg, 99%). LCMS (ES⁺) Method 2: m/z 242 (M+Na)⁺, RT 1.59 min.

### Intermediate 111: 6-chloro-3-(2,3-dichlorophenyl)-2-methylpyrimidin-4(3H)-one

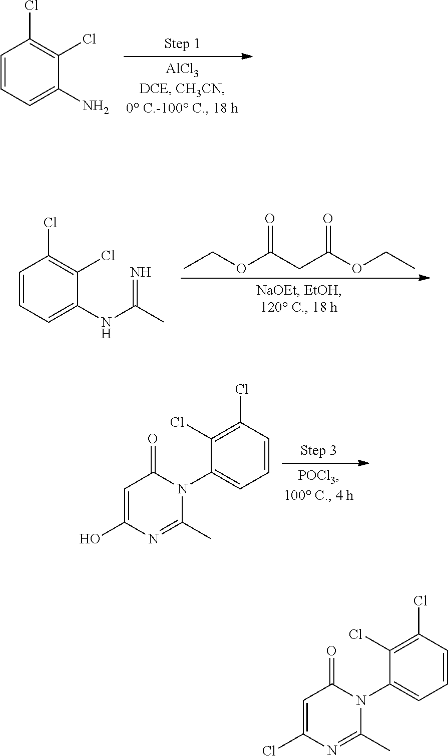 [SMILES: Nc1cccc(Cl)c1Cl ; CC(=N)Nc1cccc(Cl)c1Cl ; CCOC(=O)CC(=O)OCC ; Cc1nc(O)cc(=O)n1-c1cccc(Cl)c1Cl ; Cc1nc(Cl)cc(=O)n1-c1cccc(Cl)c1Cl]

### Step 1: N-(2,3-dichlorophenyl)acetimidamide

A solution of 2,3-dichloroaniline (1.62 g, 10.0 mmol) in dry DCE (10 mL) and MeCN (0.78 mL) was treated with aluminium chloride (1.47 g, 11 mmol) at 0° C. The reaction mixture was stirred at 0° C. for 10 min then at 100° C. for 18 h. After cooling the reaction mixture was treated with ice H₂O (30 mL) and extracted with DCM (2×). NaOH (2M aqueous sol.) was added to the mixture to adjust the pH to 10 and the aqueous phase was extracted with DCM. The combined organic layers were dried over Na₂SO₄ and concentrated in vacuo to afford the title compound as a brown oil (1.62 g, 80%) which was used as a crude without further purification. LCMS (ES⁺) Method 2: m/z 203 (M+H)⁺, RT 1.15 min.

### Step 2: 3-(2,3-dichlorophenyl)-6-hydroxy-2-methylpyrimidin-4(3H)-one

A suspension of N-(2,3-dichlorophenyl)acetimidamide (1.61 g, 7.93 mmol) in EtOH (8 mL) was treated with diethyl propanedioate (2.42 mL, 15.86 mmol) and sodium ethanolate (20% solution in EtOH; 8.88 mL, 23.78 mmol) and the resulting mixture was stirred in a sealed tube at 120° C. for 18 h. The reaction mixture was allowed to cool to 25° C. and the volatiles were removed under reduced pressure. H₂O was added to the residue, the mixture was cooled to 0° C. and acidified to pH-2 with HCl (6 M aqueous sol.). The mixture was allowed to warm to 25° C. and stirred for 1 h. The solid formed was collected by vacuum filtration, rinsed with Et₂O, and dried under reduced pressure to afford the title compound (955 mg, 44%). LCMS (ES⁺) Method 2: m/z 271 (M+H)⁺, RT 1.08 min.

### Step 3: 6-chloro-3-(2,3-dichlorophenyl)-2-methylpyrimidin-4(3H)-one

A solution of 3-(2,3-dichlorophenyl)-6-hydroxy-2-methylpyrimidin-4(3H)-one (950 mg, 3.5 mmol) in POCl₃ (10.0 mL) was heated at 100° C. for 4 h. The reaction mixture was cooled to rt and concentrated under reduced pressure. Ice H₂O was added to the mixture which was extracted with DCM. The organic layer was dried over Na₂SO₄, filtered, and concentrated in vacuo. The crude product was purified by flash chromatography (0-50% EtOAc in petroleum ether) to afford the title compound as a beige powder (500 mg, 49%). LCMS (ES⁺) Method 2: m/z 289 (M+H)⁺, RT 1.68 min.

### Intermediate 112: 8-chloro-3-(2,3-dichlorophenyl)imidazo[1,5-a]pyrazine

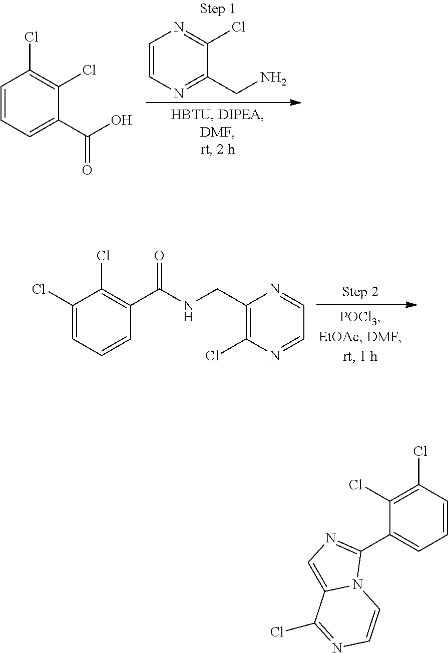 [SMILES: O=C(O)c1cccc(Cl)c1Cl ; NCc1nccnc1Cl ; O=C(NCc1nccnc1Cl)c1cccc(Cl)c1Cl ; Clc1cccc(-c2ncc3c(Cl)nccn23)c1Cl]

### Step 1: 2,3-dichloro-N-((3-chloropyrazin-2-yl)methyl)benzamide

A solution of (3-chloropyrazin-2-yl)methanamine hydrochloride (123 mg, 0.68 mmol), DIPEA (271 mg, 2.09 mmol) and HBTU (240 mg, 0.63 mmol) in DMF (1.9 mL) was stirred at rt for 2 h. The solvent was then removed under reduced pressure and the mixture dissolved in EtOAc and washed with 1N HCl, NaHCO₃ sat. sol., H₂O and brine. The organic layer was dried over Na₂SO₄, filtered and concentrated in vacuo. The crude product was purified by flash chromatography (0-100% EtOAc in petroleum ether) to afford the title compound as a white powder (110 mg, 66%). LCMS (ES⁺) Method 2: m/z 316, 318 (M+H)⁺, RT 1.52 min.

### Step 2: 8-chloro-3-(2,3-dichlorophenyl)imidazo[1,5-a]pyrazine

A suspension of 2,3-dichloro-N-((3-chloropyrazin-2-yl)methyl)benzamide (50.0 mg, 0.16 mmol) in DMF (0.26 mL) and EtOAc (0.54 mL) was treated with POCl₃ (0.07 mL, 0.79 mmol) and stirred at rt for 1 h. The solvent was removed under reduced pressure and the residue was diluted with EtOAc and ice H₂O. Na₂CO₃ sat. sol. was added, the organic phase was separated, and the aqueous phase was extracted with EtOAc. The combined organic layers were dried over Na₂SO₄, filtered, and concentrated in vacuo. The crude product was purified by flash chromatography (0-50% EtOAc in petroleum ether) to afford the title compound as a white powder (37 mg, 78%). LCMS (ES⁺) Method 2: m/z 298, 300 (M+H)⁺, RT 1.84 min.

### Intermediate 113: 4-chloro-5-(4,4,5,5-tetramethyl-1,3,2-dioxaborolan-2-yl)pyrazolo[1,5-a]pyridine

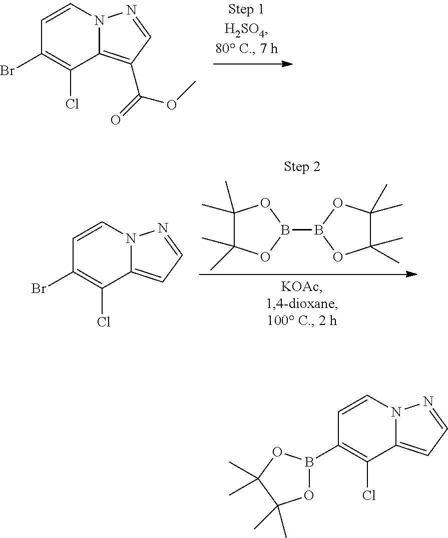

### Step 1: 5-bromo-4-chloropyrazolo[1,5-a]pyridine

A suspension of methyl 5-bromo-4-chloropyrazolo[1,5-a]pyridine-3-carboxylate (92 mg, 0.32 mmol) in H₂SO₄ (2.6 mL) was heated at 80° C. for 7 h. The reaction mixture was cooled to 0° C., diluted with H₂O and extracted with DCM. The organic layer was washed with NaHCO₃ sat. sol., dried over Na₂SO₄, filtered, and concentrated in vacuo to give the title compound as a white solid, that was used in the next step without further purification (73 mg, 99%). LCMS (ES⁺) Method 1A: m/z 231, 233 (M+H)⁺, RT 1.95 min.

### Step 2: 4-chloro-5-(4,4,5,5-tetramethyl-1,3,2-dioxaborolan-2-yl)pyrazolo[1,5-a]pyridine

A mixture of B₂pin₂ (110 mg, 0.43 mmol), 5-bromo-4-chloropyrazolo[1,5-a]pyridine (50 mg, 0.22 mmol), KOAc (64 mg, 0.65 mmol) and Pd(dppf)Cl₂·DCM (21 mg, 0.03 mmol) in degassed 1,4-dioxane (1 mL) was heated at 100° C. for 2 h. The black suspension was diluted with EtOAc, filtered over a pad of celite and concentrated in vacuo to give the crude title compound that was used in the next step without further purification. LCMS (ES⁺) Method 1A: m/z 279, 281 (M+H)⁺, RT 2.21 min.

### Intermediate 114: tert-butyl (((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

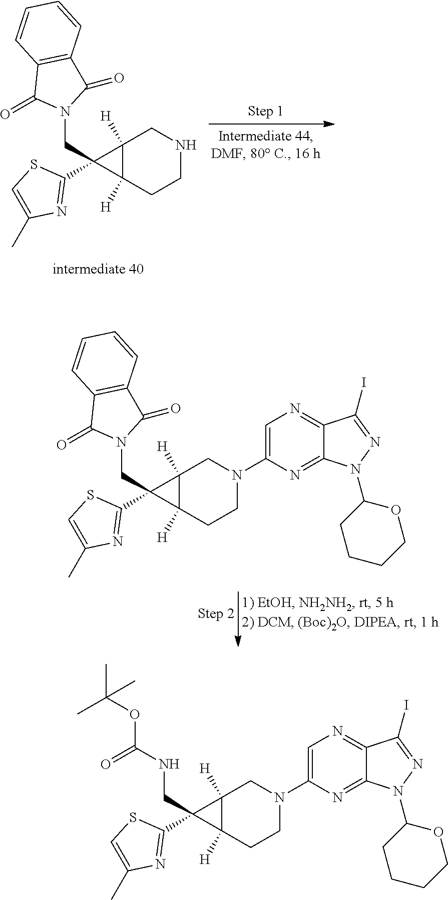 [SMILES: Cc1csc([C@@]2(CN3C(=O)c4ccccc4C3=O)[C@@H]3CCNC[C@@H]32)n1 ; Cc1csc([C@@]2(CN3C(=O)c4ccccc4C3=O)[C@@H]3CCN(c4cnc5c(I)nn(C6CCCCO6)c5n4)C[C@@H]32)n1 ; Cc1csc([C@@]2(CNC(=O)OC(C)(C)C)[C@@H]3CCN(c4cnc5c(I)nn(C6CCCCO6)c5n4)C[C@@H]32)n1]

### Step 1: 2-(((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)isoindoline-1,3-dione

A mixture of Intermediate 40 (122 mg, 0.31 mmol), Intermediate 44 (119 mg, 0.33 mmol) and dry DIPEA (0.33 mL, 1.88 mmol) in dry DMF (2.5 mL) was heated to 80° C. in a sealed vial for 16 h. The reaction was diluted with EtOAc and washed with 5% citric acid solution, H₂O, and brine. The organic layer was dried over Na₂SO₄, filtered, and evaporated to obtain a crude compound which was used in the next step without further purification (205 mg). LCMS (ES⁺) Method 1A: m/z 682 (M+H)⁺, RT 2.28 min.

### Step 2: tert-butyl (((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

2-(((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)isoindoline-1,3-dione (205 mg, 0.30 mmol) was suspended in EtOH (9 mL), N₂H₄·H₂O (0.45 mL, 9.02 mmol) was added and the reaction mixture was stirred at rt for 3 h. The volatiles were removed under reduced pressure and the residue was taken up in DCM (5 mL) then a solution of (Boc)₂O (131 mg, 0.6 mmol) in DCM (1 mL) and DIPEA (0.21 mL, 1.2 mmol) were added, and the reaction mixture was stirred at rt for 2.5 h. The reaction was diluted with DCM and washed with 5% citric acid solution (×2). The aqueous phase was extracted again with DCM then the collected organic layers were dried over Na₂SO₄, filtered, and concentrated in vacuo. The resulting crude was purified by flash chromatography (30-50% EtOAc in petroleum ether) to afford the title compound as a yellow foam (120 mg, 60% over 3 steps).

### Intermediate 115: 2-(((1S,6R,7R)-7-(2-fluorophenyl)-3-azabicyclo[4.1.0]heptan-7-yl)methyl)isoindoline-1,3-dione hydrochloride

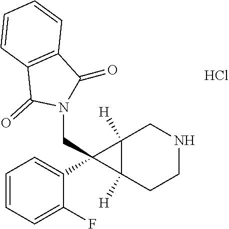 [SMILES: O=C1c2ccccc2C(=O)N1C[C@]1(c2ccccc2F)[C@@H]2CCNC[C@@H]21 ; Cl]

### Step 1: tert-butyl (1S,6R,7R)-7-((1,3-dioxoisoindolin-2-yl)methyl)-7-(2-fluorophenyl)-3-azabicyclo[4.1.0]heptane-3-carboxylate

tert-butyl 7-(aminomethyl)-7-(2-fluorophenyl)-3-azabicyclo [4.1.0]heptane-3-carboxylate (prepared as reported in the synthesis of Intermediate 3 step 1 and 2; 164 mg, 0.51 mmol) and isobenzofuran-1,3-dione (91 mg, 0.61 mmol) were dissolved in dry toluene (6 mL) then TEA (0.11 mL, 0.77 mmol) was added and the reaction was stirred at 105° C. for 24 h. The obtained mixture was cooled to rt, diluted with EtOAc, and washed with 5% citric acid solution, NaHCO₃ sat. sol. and brine. The organic layer was dried over Na₂SO₄, filtered, and concentrated in vacuo, to obtain the title compound as off-white foam, that was used in the next step without further purification (235 mg). LCMS (ES⁺) Method 1A: m/z 451 (M+H)⁺, RT 2.37 min.

### Step 2: 2-(((1S,6R,7R)-7-(2-fluorophenyl)-3-azabicyclo[4.1.0]heptan-7-yl)methyl)isoindoline-1,3-dione hydrochloride

The tile compound was synthesized using the procedure reported in the synthesis of Intermediate 3 step 4. The title compound was obtained as off-white solid. LCMS (ES⁺) Method 1A: m/z 351 (M+H)⁺, RT 1.30 min.

### Intermediate 116: tert-butyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate

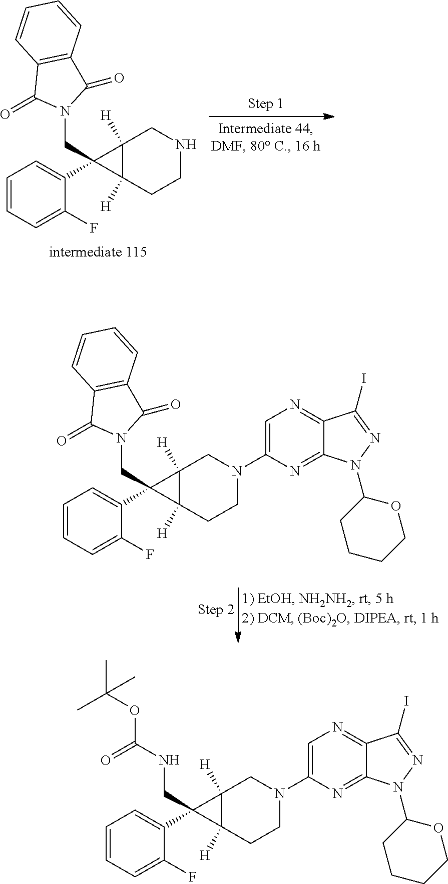 [SMILES: O=C1c2ccccc2C(=O)N1C[C@]1(c2ccccc2F)[C@@H]2CCNC[C@@H]21 ; O=C1c2ccccc2C(=O)N1C[C@]1(c2ccccc2F)[C@@H]2CCN(c3cnc4c(I)nn(C5CCCCO5)c4n3)C[C@@H]21 ; CC(C)(C)OC(=O)NC[C@]1(c2ccccc2F)[C@@H]2CCN(c3cnc4c(I)nn(C5CCCCO5)c4n3)C[C@@H]21]

### Step 1: 2-(((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione

Intermediate 115 (198 mg, 0.51 mmol) was reacted with Intermediate 44 (196 mg, 0.54 mmol) according to the procedure reported for the synthesis of Intermediate 114 step 1. The crude title compound was used in the next step without further purification (390 mg). LCMS (ES⁺) Method 1A: m/z 679 (M+H)⁺, RT 2.58 min.

### Step 2: tert-butyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate

Crude 2-(((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione (347 mg, 0.51 mmol) was suspended in EtOH (15 mL), N₂H₄·H₂O (0.76 mL, 15.4 mmol) was added and the reaction mixture was stirred at rt for 4 h. The solvent was removed under reduced pressure and the residue was dried overnight in vacuo. The residue was suspended in DCM (10 mL), then a solution of (Boc)₂O (223 mg, 1.02 mmol) in DCM (2 mL) and DIPEA (0.36 mL, 2.05 mmol) were added, and the reaction mixture was stirred at rt for 1 h. The reaction was diluted with DCM and washed with 5% citric acid solution (×2). The aqueous phase was extracted again with DCM, then the collected organic layers were dried over Na₂SO₄ filtered and concentrated in vacuo. The resulting crude was purified by flash chromatography (10-30% EtOAc in petroleum ether) to obtain the title compound as light-yellow foam (266 mg, 80% over three steps). LCMS (ES⁺) Method 1A: m/z 649 (M+H)⁺, RT 2.55 min.

### Intermediate 117: 4-chloro-2-methyl-5-(4,4,5,5-tetramethyl-1,3,2-dioxaborolan-2-yl)pyrazolo[1,5-a]pyridine

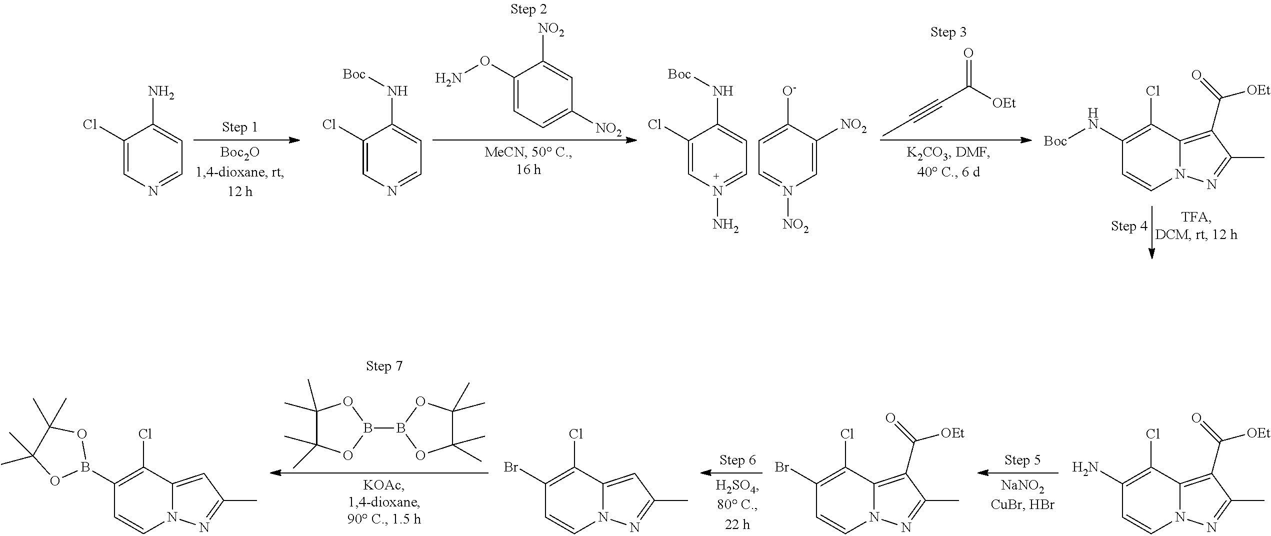

### Step 1: tert-butyl (3-chloropyridin-4-yl)carbamate

3-Chloropyridin-4-amine (3.55 g, 27.6 mmol) was suspended in 1,4-dioxane (5 mL) then (Boc)₂O (6.64 g, 30.4 mmol) was added as solid followed by 1,4-dioxane (25 mL). The brownish mixture was stirred at rt for 1 h. The solvent was partially removed to about ⅓ and the resulting suspension was stirred at rt overnight. The solvent was removed in vacuo and the residue was suspended in petroleum ether (15 mL) and filtered. The resulting solid was washed with petroleum ether (3×5 mL) then dried over Na₂SO₄, filtered, and concentrated in vacuo giving the title compound as a pale-yellow powder (5 g, 79%). 1H NMR (300 MHz, CDCl₃) δ 8.49 (s, 1H), 8.41-8.36 (d, J=5.6 Hz, 1H), 8.19-8.14 (d, J=5.6 Hz, 1H), 7.27-7.09 (br s, 1H), 1.57 (s, 9H); LCMS (ES⁺) Method 1A: m/z 229, 231 (M+H)⁺, RT 2.05 min.

### Step 2: 1-amino-4-((tert-butoxycarbonyl)amino)-3-chloropyridin-1-ium 2,4-dinitrophenolate

Tert-butyl (3-chloropyridin-4-yl)carbamate (3.2 g, 14 mmol) and O-(2,4-dinitrophenyl)hydroxylamine (5.6 g, 28 mmol) were suspended in MeCN (43 mL) and the mixture was heated at 50° C. for 16 h. The solvent was removed in vacuo to obtain the crude title compound that was used in the next step without further purification. LCMS (ES⁺) Method 1A: m/z 244, 246 (M+H)⁺, RT 0.94 min.

### Step 3: ethyl 5-((tert-butoxycarbonyl)amino)-4-chloropyrazolo[1,5-a]pyridine-3-carboxylate

Crude 1-amino-4-((tert-butoxycarbonyl)amino)-3-chloropyridin-1-ium 2,4-dinitrophenolate (6 g, 14 mmol) was dissolved in DMF (36 mL) and treated portion wise with K₂CO₃ (5.9 g, 42 mmol). The reaction mixture was stirred under N₂ atmosphere for 1 h at rt, then ethyl 2-butynoate (1.66 mL, 14.3 mmol) was added dropwise. The obtained mixture was heated at 40° C. for 6 days, then was diluted with H₂O (50 mL) and extracted with EtOAc (3×35 mL). The combined organic layers were washed with brine, dried over Na₂SO₄, filtered, and concentrated in vacuo. The resulting crude was purified by flash chromatography (5% EtOAc in petroleum ether) to give the title compound as a yellow solid (1.3 g, 26%). ¹H NMR (300 MHz, CDCl₃) δ 9.18-9.01 (br s, 1H), 8.70-8.61 (d, J=7.5 Hz, 1H), 7.47-7.40 (d, J=7.5 Hz, 1H), 4.35-4.23 (q, J=7.1 Hz, 2H), 2.46 (s, 3H), 1.50 (s, 9H), 1.36-1.28 (t, J=7.1 Hz, 3H); LCMS (ES⁺) Method 4A: m/z 354 (M+H)⁺, RT 3.36 min.

### Step 4: ethyl 5-amino-4-chloropyrazolo[1,5-a]pyridine-3-carboxylate

TFA (3.7 mL, 48.3 mmol) was added to a solution of ethyl 5-((tert-butoxycarbonyl)amino)-4-chloropyrazolo[1,5-a]pyridine-3-carboxylate (1.25 g, 3.53 mmol) in DCM (4 mL) and the reaction mixture was stirred overnight at rt. The reaction was diluted with DCM and poured portionwise into a mixture of NaHCO₃ (5.2 g), H₂O and ice. The layers were separated, and the organic phase was dried over Na₂SO₄, filtered, and concentrated in vacuo. The resulting crude was purified by flash chromatography (0-20% EtOAc in DCM) to give the title compound as a yellow solid (584 mg, 69%). ¹H NMR (300 MHz, CDCl₃) δ 8.15-8.09 (d, J=7.3 Hz, 1H), 6.42-6.36 (d, J=7.3 Hz, 1H), 4.44-4.33 (q, J=7.1 Hz, 2H), 2.56 (s, 3H), 1.49-1.37 (t, J=7.1 Hz, 3H); LCMS (ES⁺) Method 1A: m/z 254, 256 (M+H)⁺, RT 1.59 min.

### Step 5: ethyl 5-bromo-4-chloro-2-methylpyrazolo[1,5-a]pyridine-3-carboxylate

Ethyl 5-amino-4-chloro-2-methylpyrazolo[1,5-a]pyridine-3-carboxylate (300 mg, 1.18 mmol) was suspended in HBr (48% in H₂O; 4.0 mL, 35.48 mmol), cooled to 0° C. and treated with a solution of NaNO₂ (166 mg, 2.37 mmol) in H₂O (2 mL). The reaction mixture was stirred at 0° C. for 45 min then a solution of CuBr (161 mg, 1.12 mmol) in HBr (48% in H₂O; 1.3 mL, 11.23 mmol) was added giving a brown dense suspension that was warmed to rt and stirred for additional 50 min. The reaction mixture was cooled to 0° C. and poured slowly into a mix of H₂O and ice. The aqueous layer was extracted with EtOAc (×2), then the combined organic layers were dried over Na₂SO₄, filtered, and concentrated in vacuo to obtain the crude title compound as a red oil, that was used in the next step without further purification (470 mg). LCMS (ES⁺) Method 1A: m/z 317, 319 (M+H)⁺, RT 2.13 min.

### Step 6: 5-bromo-4-chloro-2-methylpyrazolo[1,5-a]pyridine

A suspension of crude ethyl 5-bromo-4-chloro-2-methylpyrazolo[1,5-a]pyridine-3-carboxylate (376 mg, 1.18 mmol) in H₂SO₄ (10 mL) was heated to 80° C. for 22 h. The reaction was then cooled to 0° C., diluted with H₂O and extracted with DCM (×2). The collected organic layers were washed with NaHCO₃ sat. sol., dried over Na₂SO₄, filtered, and concentrated in vacuo. The resulting crude was purified by preparative HPLC (20-42% MeCN/H₂O+0.10% TFA): fractions containing the pure product were combined and MeCN was removed by evaporation giving an aqueous white suspension, that was extracted with DCM. The organic layer was concentrated to give the title compound as an off-white solid (157 mg, 54% over two steps). ¹H NMR (300 MHz, CDCl₃) δ 8.09-8.03 (dd, J=0.8, 7.27 Hz, 1H), 6.79-6.73 (d, J=7.2 Hz, 1H), 6.36 (s, 1H), 2.42 (s, 3H); LCMS (ES⁺) Method 1A: m/z 245, 247 (M+H)⁺, RT 2.10 min.

### Step 7: 4-chloro-2-methyl-5-(4,4,5,5-tetramethyl-1,3,2-dioxaborolan-2-yl)pyrazolo[1,5-a]pyridine

A mixture of B₂pin₂ (108 mg, 0.42 mmol), 5-bromo-4-chloro-2-methylpyrazolo[1,5-a]pyridine (52 mg, 0.21 mmol), KOAc (62 mg, 0.64 mmol) and Pd(dppf)Cl₂·DCM (21 mg, 0.03 mmol) in degassed 1,4-dioxane (1 mL) was heated at 100° C. for 1.5 h. The black suspension was diluted with EtOAc, filtered over a pad of celite and concentrated in vacuo to give the crude title compound that was used in the next step without further purification. LCMS (ES⁺) Method 1A: m/z 293, 295 (M+H)⁺, RT 2.30 min.

### Intermediate 118: 4-(4,4,5,5-tetramethyl-1,3,2-dioxaborolan-2-yl)naphthalene-1-sulfonyl fluoride

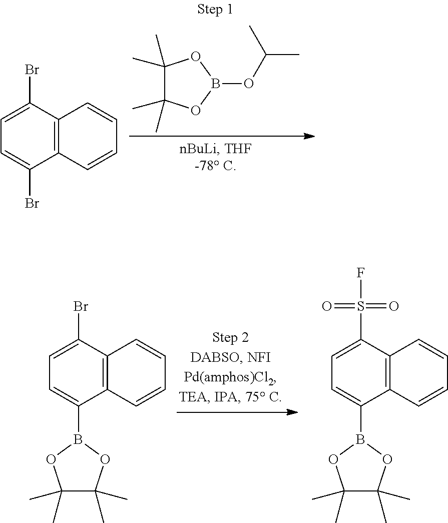

### Step 1: 2-(4-bromonaphthalen-1-yl)-4,4,5,5-tetramethyl-1,3,2-dioxaborolane

To a solution of 1,4-dibromonaphthalene (254 mg, 0.89 mmol) in dry THF (9 mL) cooled to −78° C. n-BuLi (2.5M in hexane, 0.36 mL, 0.89 mmol) was added under N₂ atmosphere. The yellow reaction mixture was stirred at −78° C. for 5 min, then 2-isopropoxy-4,4,5,5-tetramethyl-1,3,2-dioxaborolane (0.22 mL, 1.07 mmol) was added and the reaction was allowed to warm to rt, then stirred for 30 min. Et₂O and H₂O were added, the layers were separated, and the aqueous phase was extracted again with Et₂O. The combined organic extracts were dried over Na₂SO₄, filtered, and evaporated, and the resulting crude was purified by flash chromatography (C18; 40-100% MeCN/H₂O), to obtain the title compound as colourless oil that crystallized (141 mg, 48%). Method 3A: RT 2.32 min.

### Step 2: 4-(4,4,5,5-tetramethyl-1,3,2-dioxaborolan-2-yl)naphthalene-1-sulfonyl fluoride

To a mixture of 2-(4-bromonaphthalen-1-yl)-4,4,5,5-tetramethyl-1,3,2-dioxaborolane (70 mg, 0.21 mmol), DABSO (50.5 mg, 0.21 mmol) and bis(di-tert-butyl(4-dimethylaminophenyl)phosphine)dichloropalladium(II) (15 mg, 0.02 mmol) in dry degassed IPA (0.9 mL), under N₂ atmosphere, TEA (0.09 mL, 0.63 mmol) was added. The reaction mixture was stirred at 75° C. for 21 h. After cooling to rt, NFSI (73 mg, 0.23 mmol) was added, and the reaction mixture was stirred at rt for 3 h. More NFSI (73 mg, 0.23 mmol) was added, and the reaction mixture was stirred at rt for another 1 h. EtOAc was added, the reaction was filtered, and solvent removed under vacuo. The resulting crude was purified by flash chromatography (0-20% EtOAc in petroleum ether), to obtain the title compound as off-white solid (11 mg, 16%). ¹H NMR (300 MHz, CDCl₃) δ 8.94-8.87 (m, 1H), 8.61-8.48 (m, 1H), 8.32 (d, J=7.5 Hz, 1H), 8.15 (dd, J=1.4, 7.4 Hz, 1H), 7.81-7.66 (m, 2H), 1.45 (s, 12H); LCMS (ES⁺) Method 3A: RT 2.06 min.

### Intermediate 119: benzyl ((7-(4-chlorothiazol-2-yl)-3-azabicyclo[4.1.0]heptan-7-yl)methyl)carbamate hydrochloride

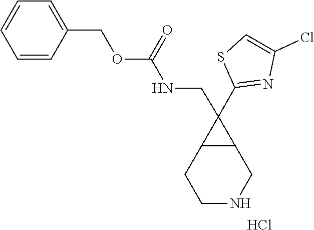 [SMILES: O=C(NCC1(c2nc(Cl)cs2)C2CCNCC21)OCc1ccccc1 ; Cl]

The title compound was synthesized using the procedure reported for Intermediate 3 using 2-(4-chlorothiazol-2-yl)acetonitrile in step 1 and N-(Benzyloxycarbonyloxy)succinimide in DCM in step 3. Intermediate 119 was obtained as a white powder. LCMS (ES⁺) Method 1A: m/z 378 (M+H)⁺, RT 1.39 min.

### Intermediate 120: 8-((tert-butyldimethylsilyl)oxy)-5-(4,4,5,5-tetramethyl-1,3,2-dioxaborolan-2-yl)quinoline

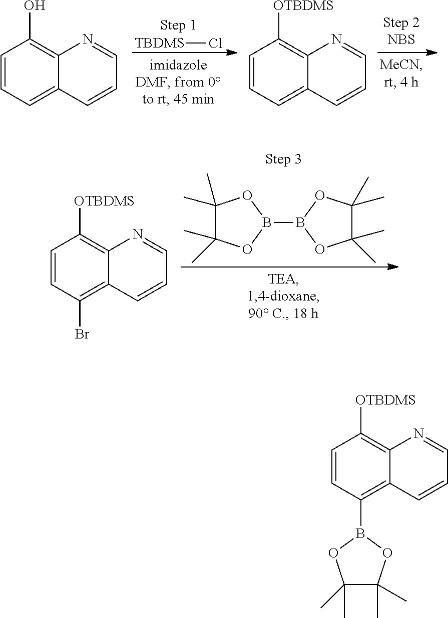

### Step 1: 8-((tert-butyldimethylsilyl)oxy)quinoline

Quinolin-8-ol (300 mg, 2.07 mmol) and 1H-imidazole (183 mg, 2.69 mmol) were dissolved in dry DMF (6 mL) at 0° C. then tert-butylchlorodimethylsilane (374 mg, 2.48 mmol) was added. The reaction was stirred at rt for 45 min, then was diluted with EtOAc and H₂O. Layers were separated and the organic phase was washed with H₂O (2×) and brine, then was dried over Na₂SO₄, filtered, and concentrated in vacuo. The resulting crude was purified by flash chromatography (0-10% EtOAc in petroleum ether) to obtain the title compound as colourless oil (520 mg, 97%). LCMS (ES⁺) Method 1A: m/z 260 (M+H)⁺, RT 1.61 min.

### Step 2: 5-bromo-8-((tert-butyldimethylsilyl)oxy)quinoline

8-((Tert-butyldimethylsilyl)oxy)quinoline (366 mg, 1.41 mmol) was dissolved in dry MeCN (9.3 mL) then NBS (264 mg, 1.48 mmol) was added portionwise at rt. The reaction was stirred at this temperature for 4 h, then was diluted with EtOAc and washed with H₂O (×2). The organic phase was dried over Na₂SO₄, filtered, and concentrated in vacuo. The resulting crude was purified by flash chromatography (0-1% EtOAc in petroleum ether) to obtain the title compound as a colourless oil (400 mg, 84%). LCMS (ES⁺) Method 1A: m/z 338, 340 (M+H)⁺, RT 2.46 min.

### Step 3: 8-((tert-butyldimethylsilyl)oxy)-5-(4,4,5,5-tetramethyl-1,3,2-dioxaborolan-2-yl)quinoline

5-Bromo-8-((tert-butyldimethylsilyl)oxy)quinoline (110 mg, 0.33 mmol), B₂pin₂(165 mg, 0.65 mmol) and Pd(dppf)Cl₂·DCM (33.2 mg, 0.04 mmol) were sequentially dissolved in degassed 1,4-dioxane (1.1 mL) then TEA (0.18 mL, 1.3 mmol) was added. The reaction mixture was heated at 100° C. for 18 h. The black suspension was diluted with H₂O and EtOAc, then the organic layer was washed with brine (×2), dried over Na₂SO₄, filtered, and concentrated in vacuo. The resulting crude was purified by flash chromatography (0-2% EtOAc/petroleum ether) to give the title compound as a pale-yellow oil. (54 mg, 43%). LCMS (ES⁺) Method 1A: m/z 386 (M+H)⁺, RT 2.08 min

### Intermediate 122: 2-(((1R,5S,6r)-6-(4-methylthiazol-2-yl)-3-azabicyclo[3.1.0]hexan-6-yl)methyl)isoindoline-1,3-dione hydrochloride

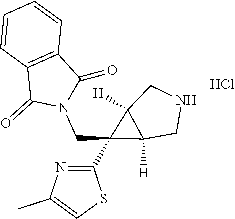 [SMILES: Cc1csc([C@@]2(CN3C(=O)c4ccccc4C3=O)[C@@H]3CNC[C@@H]32)n1 ; Cl]

### Step 1: tert-butyl (1R,5S,6r)-6-((1,3-dioxoisoindolin-2-yl)methyl)-6-(4-methylthiazol-2-yl)-3-azabicyclo[3.1.0]hexane-3-carboxylate

Tert-butyl (1R,5S,6r)-6-(aminomethyl)-6-(4-methylthiazol-2-yl)-3-azabicyclo[3.1.0]hexane-3-carboxylate (prepared as reported for the synthesis of Intermediate 94 step 1-4, using (4-methyl-1,3-thiazol-2-yl)acetonitrile) (184 mg, 0.59 mmol) and isobenzofuran-1,3-dione (106 mg, 0.71 mmol) were dissolved in toluene (7 mL) then TEA (0.12 mL, 0.89 mmol) was added and the reaction was stirred at 105° C. for 8 h. The reaction mixture was cooled to rt, diluted with EtOAc, and washed with 5% citric acid sol., NaHCO₃ sat. sol. and brine. The organic layer was dried over Na₂SO₄, filtered, and concentrated in vacuo to obtain the title compound as light-brown foam, which was used in the next step without further purification. LCMS (ES⁺) Method 1A: m/z 440 (M+H)⁺, RT 1.98 min.

### Step 2: 2-(((1R,5S,6r)-6-(4-methylthiazol-2-yl)-3-azabicyclo[3.1.0]hexan-6-yl)methyl)isoindoline-1,3-dione hydrochloride

The title compound was synthesized using the procedure reported for Intermediate 94 step 6 and obtained as beige solid. LCMS (ES⁺) Method 1A: m/z 340 (M+H)⁺, RT 1.05 min.

### Intermediate 123: tert-butyl (((1R,5S,6r)-3-(3-iodo-1-(tetrahydro-2H-pyran-2-yl)-1H-pyrazolo[3,4-b]pyrazin-6-yl)-6-(4-methylthiazol-2-yl)-3-azabicyclo[3.1.0]hexan-6-yl)methyl)carbamate

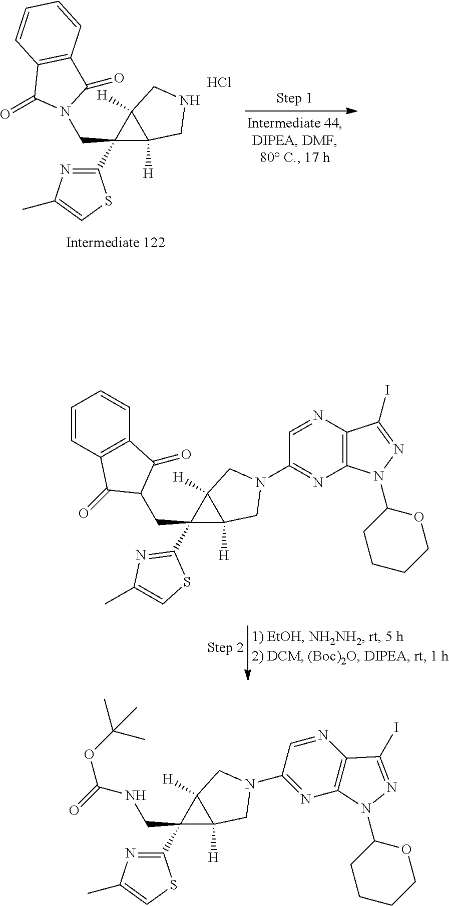 [SMILES: Cc1csc([C@@]2(CN3C(=O)c4ccccc4C3=O)[C@@H]3CNC[C@@H]32)n1 ; Cl ; Cc1csc([C@@]2(CC3C(=O)c4ccccc4C3=O)[C@@H]3CN(c4cnc5c(I)nn(C6CCCCO6)c5n4)C[C@@H]32)n1 ; Cc1csc([C@@]2(CNC(=O)OC(C)(C)C)[C@@H]3CN(c4cnc5c(I)nn(C6CCCCO6)c5n4)C[C@@H]32)n1]

### Step 1: 2-(((1R,5S,6r)-3-(3-iodo-1-(tetrahydro-2H-pyran-2-yl)-1H-pyrazolo[3,4-b]pyrazin-6-yl)-6-(4-methylthiazol-2-yl)-3-azabicyclo[3.1.0]hexan-6-yl)methyl)isoindoline-1,3-dione

Intermediate 122 (185 mg, 0.49 mmol) and Intermediate 44 (198 mg, 0.54 mmol) were reacted according to the procedure reported for the synthesis of Intermediate 114 step 1. The compound was obtained as off-white foam (367 mg). LCMS (ES⁺) Method 1A: m/z 668 (M+H)⁺, RT 2.2 min.

### Step 2: tert-butyl (((1R,5S,6r)-3-(3-iodo-1-(tetrahydro-2H-pyran-2-yl)-1H-pyrazolo[3,4-b]pyrazin-6-yl)-6-(4-methylthiazol-2-yl)-3-azabicyclo[3.1.0]hexan-6-yl)methyl)carbamate

2-(((1R,5S,6r)-3-(3-iodo-1-(tetrahydro-2H-pyran-2-yl)-1H-pyrazolo[3,4-b]pyrazin-6-yl)-6-(4-methylthiazol-2-yl)-3-azabicyclo[3.1.0]hexan-6-yl)methyl)isoindoline-1,3-dione (329 mg, 0.049 mmol) was suspended in EtOH (15 mL) and N₂H₄·H₂O (0.73 mL, 14.8 mmol) was added. The reaction mixture was stirred at rt for 5 h then the solvent was concentrated in vacuo. The residue was suspended in DCM (11 mL) then a solution of (Boc)₂O (215 mg, 0.99 mmol) in DCM (1.5 mL) and DIPEA (0.34 mL, 1.97 mmol) was added to the previous one and the obtained mixture was stirred at rt for 1 h. After reaction completion, DCM was added, and the organic solvent was washed with 5% citric acid solution (×2). Organic layer was dried over Na₂SO₄, filtered, and concentrated in vacuo. The resulting crude was purified by flash chromatography on silica gel (from 0% to 50% EtOAc in petroleum ether) to give the title compound as yellow foam (260 mg, 83% over three steps). LCMS (ES⁺) Method 1A: m/z 638 (M+H)⁺, RT 2.24 min.

### Intermediate 126: benzyl (((1R,5S,6r)-6-(1,2,5-thiadiazol-3-yl)-3-azabicyclo[3.1.0]hexan-6-yl)methyl)carbamate hydrochloride

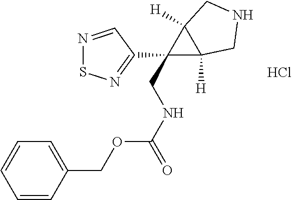 [SMILES: O=C(NC[C@]1(c2cnsn2)[C@@H]2CNC[C@@H]21)OCc1ccccc1 ; Cl]

The title compound was synthesized according to the procedure reported for Intermediate 94 using 2-(1,2,5-thiadiazol-3-yl)acetonitrile in step 3, and ZnCl₂ and obtained as white solid. LCMS (ES⁺) Method 1A: m/z 431 (M+H)⁺, RT 2.15 min.

### Intermediate 128: benzyl (((1R,5S,6r)-6-(6-chloropyridin-2-yl)-3-azabicyclo[3.1.0]hexan-6-yl)methyl)carbamate

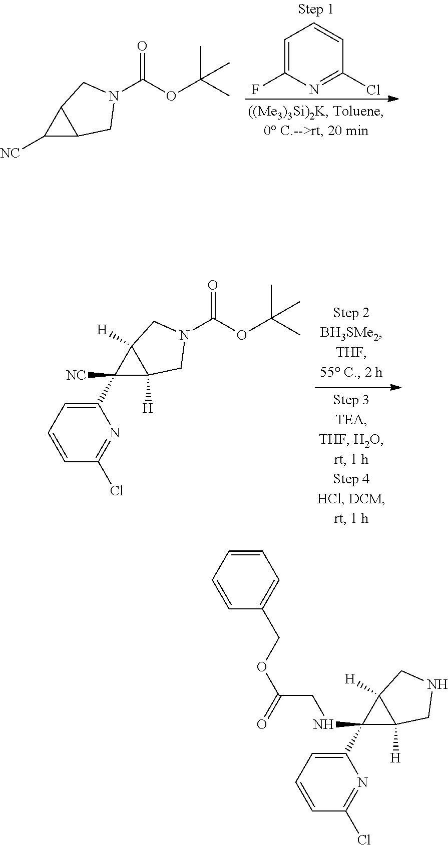

### Step 1: tert-butyl (1R,5S,6s)-6-(6-chloropyridin-2-yl)-6-cyano-3-azabicyclo[3.1.0]hexane-3-carboxylate

Potassium bis(trimethylsilyl)amide (0.5M solution in toluene) (1 mL, 0.5 mmol) was added to a solution of tert-butyl 6-cyano-3-azabicyclo[3.1.0]hexane-3-carboxylate (100 mg, 0.480 mmol) and 2-chloro-6-fluoropyridine (69 mg, 0.53 mmol) in dry toluene (1.5 mL) at 0° C. The mixture was allowed to warm-up to rt and stirred for 20 min. After completion toluene was added, and the reaction mixture was washed with H₂O (1×) and brine (1×). The organic layer was dried over Na₂SO₄, filtered, and concentrated in vacuo to afford the title compound as a yellow oil, that was used in the next step without further purification (149 mg). LCMS (ES⁺) Method 1A: m/z 320 (M+H)⁺, RT 2.08 min.

### Step 2: tert-butyl (1R,5S,6r)-6-(aminomethyl)-6-(6-chloropyridin-2-yl)-3-azabicyclo[3.1.0]hexane-3-carboxylate

The title compound was prepared following the procedure reported in the synthesis of Intermediate 6 step 2. The title compound was used as a crude. LCMS (ES⁺) Method 1A: m/z. 324 (M+H)⁺, RT 1.26 min.

### Step 3: tert-butyl (1R,5S,6r)-6-((((benzyloxy)carbonyl)amino)methyl)-6-(6-chloropyridin-2-yl)-3-azabicyclo[3.1.0]hexane-3-carboxylate

The title compound was prepared following the procedure reported in the synthesis of Intermediate 6 step 3 using N-(Benzyloxycarbonyloxy)succinimide in a mixture of THF and H₂O and obtained as a white solid (157 mg, 74%). LCMS (ES⁺) Method 1A: m/z 458 (M+H)⁺, RT 2.29 min.

### Step 4: benzyl (((1R,5S,6r)-6-(6-chloropyridin-2-yl)-3-azabicyclo[3.1.0]hexan-6-yl)methyl)carbamate

The title compound was prepared following procedure reported in the synthesis of Intermediate 6 step 4 and used in the next step without further purification. LCMS (ES⁺) Method 1A: m/z 358 (M+H)⁺, RT 1.35 min.

The following compounds were synthesized according to the procedure reported in step 1 for Intermediate 128 using the appropriately substituted 2-fluoropyridine reagents.

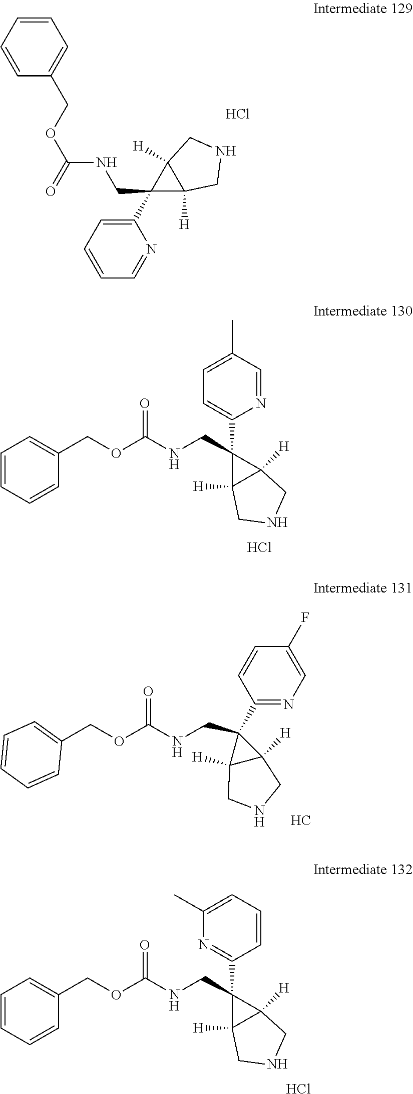

### Intermediate 133: 2-(((1R,5S,6r)-6-(isothiazol-5-yl)-3-azabicyclo[3.1.0]hexan-6-yl)methyl)isoindoline-1,3-dione hydrochloride

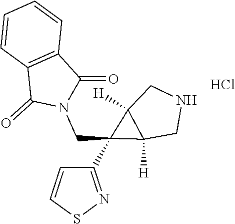 [SMILES: O=C1c2ccccc2C(=O)N1C[C@]1(c2ccsn2)[C@@H]2CNC[C@@H]21 ; Cl]

### Step 1: tert-butyl (1R,5S,6r)-6-((1,3-dioxoisoindolin-2-yl)methyl)-6-(isothiazol-3-yl)-3-azabicyclo[3.1.0]hexane-3-carboxylate

Tert-butyl (1R,5S,6r)-6-(aminomethyl)-6-(isothiazol-5-yl)-3-azabicyclo[3.1.0]hexane-3-carboxylate (prepared as reported for Intermediate 94 using (4-methyl-1,3-thiazol-2-yl)acetonitrile)) (96 mg, 0.33 mmol) and isobenzofuran-1,3-dione (58 mg, 0.39 mmol) were dissolved in dry toluene (4 mL) then TEA (0.07 mL, 0.49 mmol) was added and the reaction was stirred at 105° C. for 16 h. The obtained mixture was cooled to rt, diluted with EtOAc, and washed with 5% citric acid solution, NaHCO₃ sat. sol. and brine. The organic layer was dried over Na₂SO₄, filtered, and concentrated in vacuo. The resulting crude was purified by flash chromatography (10-50% EtOAc in petroleum ether). The title compound was obtained as white foam (97 mg, 70%). ¹H NMR (300 MHz, CDCl₃) δ 8.48 (d, J=4.7 Hz, 1H), 7.80-7.72 (m, 2H), 7.72-7.63 (m, 2H), 7.16 (d, J=4.7 Hz, 1H), 4.25-4.11 (m, 2H), 4.10-3.95 (m, 2H), 3.84-3.67 (m, 2H), 2.40-2.28 (m, 1H), 2.28-2.17 (m, 1H), 1.56 (s, 3H), 1.51 (s, 9H); LCMS (ES⁺) Method 1A: m/z 426 (M+H)⁺, RT 2.04 min.

### Step 2: 2-(((1R,5S,6r)-6-(isothiazol-3-yl)-3-azabicyclo[3.1.0]hexan-6-yl)methyl)isoindoline-1,3-dione hydrochloride

The tile compound was synthesized using the procedure reported for Intermediate 94 step 6 and obtained as off-white solid. LCMS (ES⁺) Method 1A: m/z 326 (M+H)⁺, RT 1.01 min.

### Intermediate 134: 6-chloro-5-(4,4,5,5-tetramethyl-1,3,2-dioxaborolan-2-yl)pyrazolo[1,5-a]pyridine

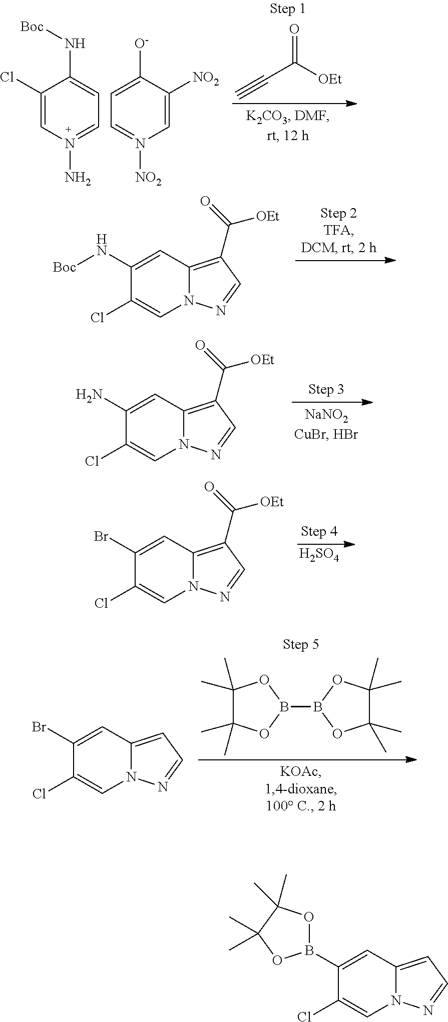

### Step 1: ethyl 5-((tert-butoxycarbonyl)amino)-6-chloropyrazolo[1,5-a]pyridine-3-carboxylate

1-Amino-4-((tert-butoxycarbonyl)amino)-3-chloropyridin-1-ium 2,4-dinitrophenolate (3.6 g, 8.5 mmol) was dissolved in DMF (22 mL) and treated with K₂CO₃ (5.2 g, 37.1 mmol) portionwise. The resulting mixture was stirred under N₂ atmosphere for 1 h at rt. Then ethyl propiolate (1.3 mL, 12.6 mmol) was added dropwise and the reaction mixture was stirred at rt overnight. The reaction was diluted with 50 mL of H₂O and extracted with EtOAc (3×35 mL). The combined organic layers were washed with brine, dried over Na₂SO₄, filtered, and concentrated in vacuo. The resulting crude was purified by flash chromatography (5% EtOAc in petroleum ether) to give the title compound (210 mg, 7%). LCMS (ES⁺) Method 4A: m/z 340 (M+H)⁺, RT 3.36 min.

### Step 2: ethyl 5-amino-6-chloropyrazolo[1,5-a]pyridine-3-carboxylate

Ethyl 5-((tert-butoxycarbonyl)amino)-6-chloropyrazolo[1,5-a]pyridine-3-carboxylate (210 mg, 0.62 mmol) was dissolved in DCM (2 mL), TFA (2 mL, 26.6 mmol) was added and the resulting solution was stirred at rt for 2 h. The reaction was then poured portionwise into a mixture of NaHCO₃ sat. sol., H₂O, and ice. Layers were separated and the aqueous phase was extracted again with DCM. The collected organic layers were dried over Na₂SO₄, filtered, and concentrated. The resulting crude was purified by flash chromatography (0-5% EtOAc in DCM), to obtain the title compound as yellow solid (98 mg, 66%). LCMS (ES⁺) Method 1A: m/z 240 (M+H)⁺, RT 1.63 min.

### Step 3: ethyl 5-bromo-6-chloropyrazolo[1,5-a]pyridine-3-carboxylate

Ethyl 5-amino-6-chloropyrazolo[1,5-a]pyridine-3-carboxylate (226 mg, 0.94 mmol) was suspended in HBr (in 48% H₂O; 3.2 mL, 28.3 mmol), cooled to 0° C. and treated with a solution of NaNO₂ (132 mg, 1.89 mmol) in H₂O (1.6 mL). The reaction mixture was stirred at 0° C. for 45 min, then CuBr (122 mg, 0.85 mmol) in HBr (in 48% H₂O; 1 mL, 9.0 mmol) was added giving a brown dense suspension that was warmed to rt and stirred for 50 min. The reaction mixture was then cooled to 0° C. and poured slowly into a mix of H₂O and ice. The aqueous phase was extracted twice with EtOAc, then the combined organic layers were dried over Na₂SO₄, filtered, and concentrated in vacuo. The resulting crude was purified by flash chromatography (50-100% DCM in petroleum ether) to obtain the title compound as light-yellow solid (130 mg, 45%). ¹H NMR (300 MHz, CDCl₃) δ 8.67 (br d, J=0.6 Hz, 1H), 8.52 (s, 1H), 8.40 (s, 1H), 4.42 (q, J=7.1 Hz, 2H), 1.44 (t, J=7.1 Hz, 4H); LCMS (ES⁺) Method 1A: m/z 303, 305 (M+H)⁺, RT 2.17 min.

### Step 4: 5-bromo-6-chloropyrazolo[1,5-a]pyridine

A suspension of ethyl 5-bromo-6-chloropyrazolo[1,5-a]pyridine-3-carboxylate (130 mg, 0.43 mmol) in H₂SO₄ (4 mL, 30 mmol) was heated at 80° C. for 28 h, then at 100° C. for another 24 h. The reaction was then cooled to 0° C., diluted with H₂O and extracted with DCM (2×). The collected organic layers were washed with NaHCO₃ sat. sol., dried over Na₂SO₄, filtered, and concentrated in vacuo. The resulting crude was purified by flash chromatography (0-4% EtOAc in petroleum ether) to obtain the title compound as white solid (63 mg, 64%). ¹H NMR (300 MHz, CDCl₃) δ 8.63 (s, 1H), 7.97 (d, J=2.3 Hz, 1H), 7.90 (s, 1H), 6.53 (d, J=1.7 Hz, 1H); LCMS (ES⁺) Method 1A: m/z 231, 233 (M+H)⁺, RT 1.91 min.

### Step 5: 6-chloro-5-(4,4,5,5-tetramethyl-1,3,2-dioxaborolan-2-yl)pyrazolo[1,5-a]pyridine

A mixture of B₂pin₂ (110 mg, 0.43 mmol), 5-bromo-6-chloropyrazolo[1,5-a]pyridine (50 mg, 0.22 mmol), KOAc (64 mg, 0.65 mmol) and Pd(dppf)Cl₂·DCM (21.2 mg, 0.03 mmol) in degassed 1,4-dioxane (1 mL) was heated at 100° C. for 1 h and 40 min. The black suspension was cooled to rt, diluted with EtOAc, filtered over a pad of celite and concentrated in vacuo to give the crude title compound, that was used in the next step without further purification. LCMS (ES⁺) Method 1A: m/z 279 (M+H)⁺, RT 2.16 min.

### Intermediate 135: 1-(3-chloro-4-(4,4,5,5-tetramethyl-1,3,2-dioxaborolan-2-yl)phenyl)-1H-pyrrole

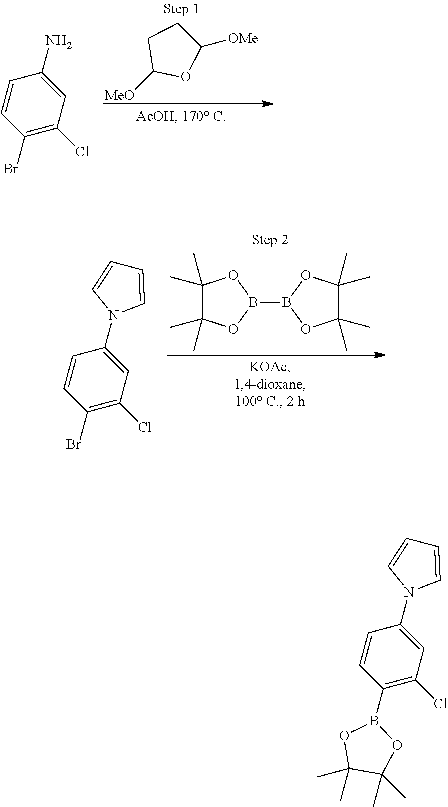

### Step 1: 1-(4-bromo-3-chlorophenyl)-1H-pyrrole

A solution of 4-bromanyl-3-chloranyl-aniline (82 mg, 0.4 mmol) and 2,5-dimethoxyoxolane (63 mg, 0.48 mmol) in AcOH (0.45 mL, 0.88 mol) was heated under MW at 170° C. for 15 min. After cooling to rt, the reaction mixture was treated with H₂O and the aqueous layer was extracted with DCM, DCM/MeOH 9:1 and EtOAc/MeOH 9:1. The combined organic phases were dried over Na₂SO₄, filtered, and concentrated in vacuo to obtain the crude title compound as dark oil, that was used in the next step without further purification. LCMS (ES⁺) Method 1A: 256, 258 (M+H)⁺, RT 2.42 min.

### Step 2: 1-(3-chloro-4-(4,4,5,5-tetramethyl-1,3,2-dioxaborolan-2-yl)phenyl)-1H-pyrrole

A mixture of crude 1-(4-bromo-3-chlorophenyl)-1H-pyrrole (102 mg, 0.4 mmol), B₂pin₂ (202 mg, 0.79 mmol), KOAc (117 mg, 1.19 mmol) and Pd(dppf)Cl₂·DCM (39 mg, 0.05 mmol) in degassed 1,4-dioxane (1.9 mL) was heated at 100° C. for 1.5 h. The black suspension was cooled to rt, diluted with EtOAc, and washed with brine (×2). Organic layer was dried over Na₂SO₄, filtered, and concentrated in vacuo to obtain the crude title compound, that was used in the next step without further purification. LCMS (ES⁺) Method 1A: 304 (M+H)⁺, RT 2.54 min.

### Intermediate 136: 4-chloro-5-(4,4,5,5-tetramethyl-1,3,2-dioxaborolan-2-yl)benzo[c]isothiazole

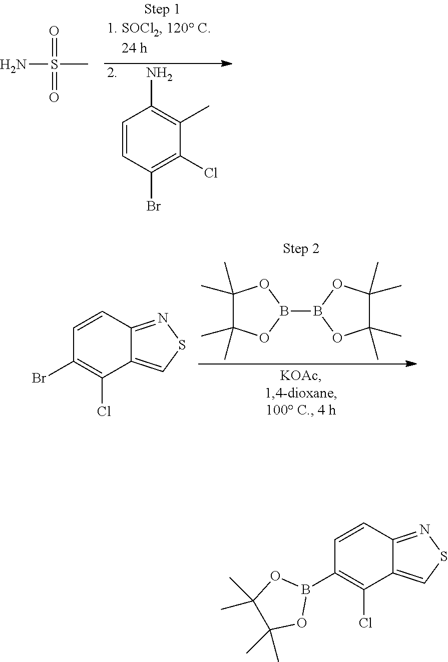

### Step 1: 5-bromo-4-chlorobenzo[c]isothiazole

Methanesulfonamide (625 mg, 6.57 mmol) and SOCl₂ (0.7 mL, 9.65 mmol) were dissolved in toluene (1.6 mL) and stirred for 24 h at 120° C. under N₂ atmosphere. After cooling to rt, the solvent was removed in vacuo and the resulting N-sulfinylmethanesulfonamide was directly used in the next step without further purification.

4-Bromo-3-chloro-2-methylaniline (207 mg, 0.94 mmol) was dissolved in toluene (3 mL) and SOCl₂ (0.075 mL, 1.03 mmol) was added dropwise at 0° C. then the temperature was raised to 120° C. and stirring was continued for 18 h. The reaction mixture was then cooled to rt, concentrated in vacuo and dissolved in toluene (0.9 mL). To this solution, the previously prepared N-sulfinylmethanesulfonamide was added followed by dry pyridine (0.11 mL, 1.41 mmol). The mixture was heated to reflux and stirred at 120° C. for 5.5 h. The reaction was cooled to rt, diluted with EtOAc then washed with H₂O (×2) and brine. The organic phase was dried over Na₂SO₄, filtered, concentrated in vacuo, and the resulting crude was purified by flash chromatography (0-10% EtOAc in petroleum ether), to obtain the title product as off-white solid (120 mg, 51%). ¹H NMR (300 MHz, CDCl₃) δ 9.25 (br d, J=0.8 Hz, 1H), 7.62-7.49 (m, 2H); LCMS (ES⁺) Method 1A: m/z 248, 250 (M+H)⁺, RT 2.13 min.

### Step 2: 4-chloro-5-(4,4,5,5-tetramethyl-1,3,2-dioxaborolan-2-yl)benzo[c]isothiazole

B₂pin₂ (123 mg, 0.48 mmol), 5-bromo-4-chlorobenzo[c]isothiazole (60 mg, 0.24 mmol), KOAc (71 mg, 0.720 mmol) and Pd(dppf)Cl₂·DCM (24 mg, 0.03 mmol) were dissolved in degassed 1,4-dioxane (1.7 mL, 0.02 mol). The mixture was heated at 100° C. for 4 h. The black suspension was then diluted with EtOAc, filtered over a pad of celite and concentrated in vacuo to give the title compound that was used in the next step without further purification. LCMS (ES⁺) Method 1A: m/z 296 (M+H)⁺, RT 2.29 min.

### Intermediate 137a and 137b: 5-bromo-4-chloro-2-(difluoromethyl)-2H-indazole and 5-bromo-4-chloro-1-(difluoromethyl)-1H-indazole

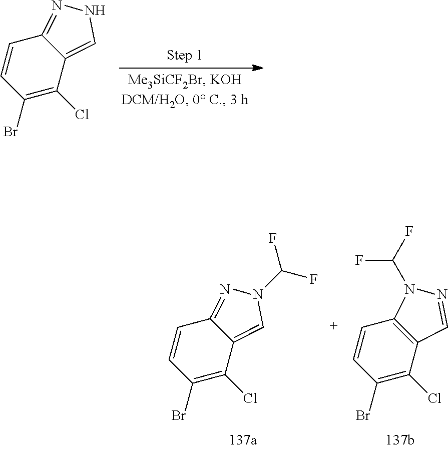 [SMILES: Clc1c(Br)ccc2n[nH]cc12 ; FC(F)n1cc2c(Cl)c(Br)ccc2n1 ; FC(F)n1ncc2c(Cl)c(Br)ccc21]

5-Bromo-4-chloro-1H-indazole (82 mg, 0.35 mmol) was suspended in DCM, cooled to 0° C. and a KOH aq. sol. (119 mg, 2.13 mmol in 0.55 mL of H₂O) was added with vigorous stirring. (Bromodifluoromethyl)trimethylsilane (0.13 mL, 0.71 mmol) was added and the reaction mixture was stirred at 0° C. for 3 h. The reaction mixture was diluted with DCM and H₂O, then the organic layer was dried over Na₂SO₄, filtered, and concentrated in vacuo. The resulting crude was purified by flash chromatography (0-20% EtOAc in petroleum ether) to obtain Intermediate 137b (first eluted, 35 mg, 35%) as a white solid, Method 1A RT 2.23 min., and Intermediate 137a (second eluted, 51 mg, 51%) as a white solid, Method 1A RT 2.13 min.

### Intermediate 137c: 4-chloro-2-(difluoromethyl)-5-(4,4,5,5-tetramethyl-1,3,2-dioxaborolan-2-yl)-2H-indazole

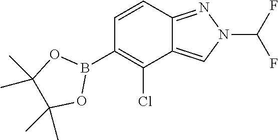 [SMILES: CC1(C)OB(c2ccc3nn(C(F)F)cc3c2Cl)OC1(C)C]

Intermediate 137c was synthesized using the procedure reported for Intermediate 15 starting from Intermediate 137a and used in the next step without further purification. LCMS (ES⁺) Method 1A: 329 (M+H)⁺, RT 2.27 min.

### Intermediate 137d: 4-chloro-1-(difluoromethyl)-5-(4,4,5,5-tetramethyl-1,3,2-dioxaborolan-2-yl)-1H-indazole

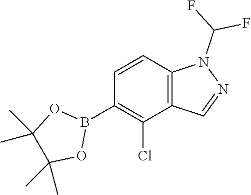 [SMILES: CC1(C)OB(c2ccc3c(cnn3C(F)F)c2Cl)OC1(C)C]

Intermediate 137d was synthesized using the procedure reported for Intermediate 15, starting from 5-bromo-4-chloro-1-(difluoromethyl)-1H-indazole (Intermediate 137b) and used in the next step without further purification. Method 1A: m/z 329 (M+H)⁺, RT 2.39 min.

### Intermediate 138: 2-(difluoromethyl)-4-fluoro-5-(4,4,5,5-tetramethyl-1,3,2-dioxaborolan-2-yl)-2H-indazole

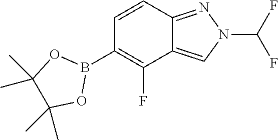 [SMILES: CC1(C)OB(c2ccc3nn(C(F)F)cc3c2F)OC1(C)C]

The title compound was prepared according to the procedure described for intermediate 137c starting from the 5-bromo-4-chloro-1H-indazole and used in the next step without further purification. LCMS (ES⁺) Method 1A: 313 (M+H)⁺, RT 2.11 min.

### Intermediate 139: 4-chloro-5-(4,4,5,5-tetramethyl-1,3,2-dioxaborolan-2-yl)benzo[c][1,2,5]thiadiazole

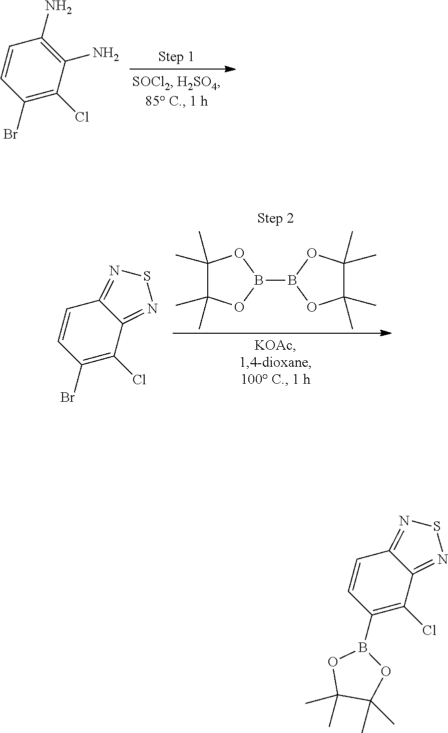

### Step 1: 5-bromo-4-chlorobenzo[c][1,2,5]thiadiazole

4-Bromo-3-chlorobenzene-1,2-diamine (70 mg, 0.32 mmol) was suspended in SOCl₂ (0.3 mL, 4.11 mmol), then H₂SO₄ (0.01 mL, 0.190 mmol) was added, and the reaction mixture was stirred at 85° C. for 1 h. After completion, the reaction mixture was cooled then poured into iced H₂O and DCM was added. The aqueous layer was extracted again with additional DCM, then the collected organic phases were washed with NaHCO₃ sat. sol. The organic layer was dried over Na₂SO₄, filtered, and concentrated in vacuo. The resulting crude was purified by flash chromatography (0-10% EtOAc in petroleum ether). The title compound was obtained as off-white powder (70 mg, 89%). ¹H NMR (300 MHz, DMSO-d₆) δ 8.10-8.01 (m, 2H); LCMS (ES⁺) Method 1A: RT 2.07 min.

### Step 2: 4-chloro-5-(4,4,5,5-tetramethyl-1,3,2-dioxaborolan-2-yl)benzo[c][1,2,5]thiadiazole

The above intermediate was prepared according to the procedure described for Intermediate 15 starting 5-bromo-4-chlorobenzo[c][1,2,5]thiadiazole and used in the next step without further purification. LCMS (ES⁺) Method 1A: RT 2.34 min.

### Intermediate 140: 2-(((1R,5S,6r)-6-(4-(trifluoromethyl)thiazol-2-yl)-3-azabicyclo[3.1.0]hexan-6-yl)methyl)isoindoline-1,3-dione hydrochloride

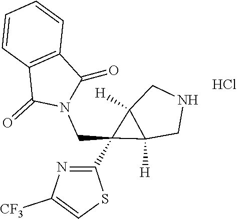 [SMILES: O=C1c2ccccc2C(=O)N1C[C@]1(c2nc(C(F)(F)F)cs2)[C@@H]2CNC[C@@H]21 ; Cl]

Intermediate 140 was synthesized using the procedure reported for Intermediate 122 using 2-(4-(trifluoromethyl)thiazol-2-yl)acetonitrile in step 3 and obtained as a light-yellow solid. LCMS (ES⁺) Method 1A: m/z 394 (M+H)⁺, RT 1.28 min.

### Intermediate 141: (3-chloro-2-methoxypyridin-4-yl)boronic acid

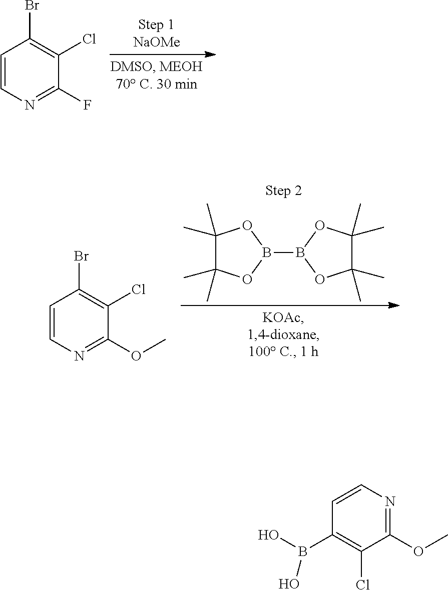

### Step 1: 4-bromo-3-chloro-2-methoxypyridine

4-Bromo-3-chloro-2-fluoro-pyridine (150 mg, 0.71 mmol) was dissolved in DMSO (1.9 mL) and MeOH (2 mL) then NaOMe (25 wt. % sol. in MeOH, 0.49 mL, 2.14 mmol) was added and the reaction mixture was stirred at 70° C. for 30 min. The reaction was diluted with H₂O and EtOAc, then the organic layer was washed with H₂O, dried over Na₂SO₄, filtered, and concentrated in vacuo to obtain the title compound as a white solid, that was used in the next step without further purification (143 mg). LCMS (ES⁺) Method 1A: m/z 222, 224 (M+H)⁺, RT 1.98 min.

### Step 2: (3-chloro-2-methoxypyridin-4-yl)boronic acid

The above intermediate was prepared according to the procedure described for Intermediate 15 starting from 4-bromo-3-chloro-2-methoxypyridine and used in the next step without further purification. LCMS (ES⁺) Method 1A: m/z 188 (M+H)⁺, RT 0.94 min.

### Intermediate 142: 2-(4-chloro-5-(4,4,5,5-tetramethyl-1,3,2-dioxaborolan-2-yl)-2H-indazol-2-yl)acetonitrile

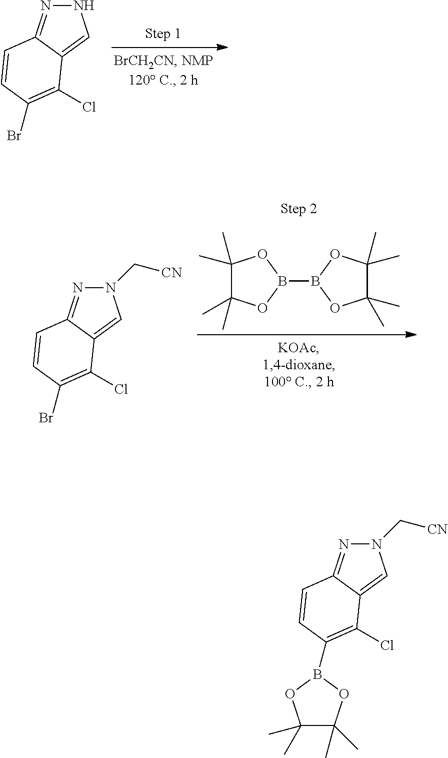

### Step 1: 2-(5-bromo-4-chloro-2H-indazol-2-yl)acetonitrile

To a suspension of 5-bromo-4-chloro-1H-indazole (154 mg, 0.67 mmol) in NMP (0.07 mL) 2-bromoacetonitrile (0.15 mL, 2.15 mmol) was added and the reaction mixture was stirred at 120° C. for 2 h. The reaction was cooled to rt and diluted with EtOAc and H₂O. The organic layer was washed with H₂O and brine, dried over Na₂SO₄, filtered, and concentrated in vacuo. The resulting crude was purified by flash chromatography (0-20% EtOAc in petroleum ether and then (50-100% DCM in petroleum ether) to give the title compound as grey solid (100 mg, 56%). LCMS (ES⁺) Method 1A: m/z 270, 272 (M+H)⁺, RT 1.90 min.

### Step 2: 2-(4-chloro-5-(4,4,5,5-tetramethyl-1,3,2-dioxaborolan-2-yl)-2H-indazol-2-yl)acetonitrile

The above intermediate was prepared according to the procedure described for Intermediate 15 starting from 2-(5-bromo-4-chloro-2H-indazol-2-yl)acetonitrile and used in the next step without further purification. LCMS (ES⁺) Method 1A: m/z 318 (M+H)⁺, RT 2.05 min.

### Intermediate 143: 2-(4-chloro-5-(4,4,5,5-tetramethyl-1,3,2-dioxaborolan-2-yl)-2H-indazol-2-yl)acetonitrile

### Step 1 Step 3

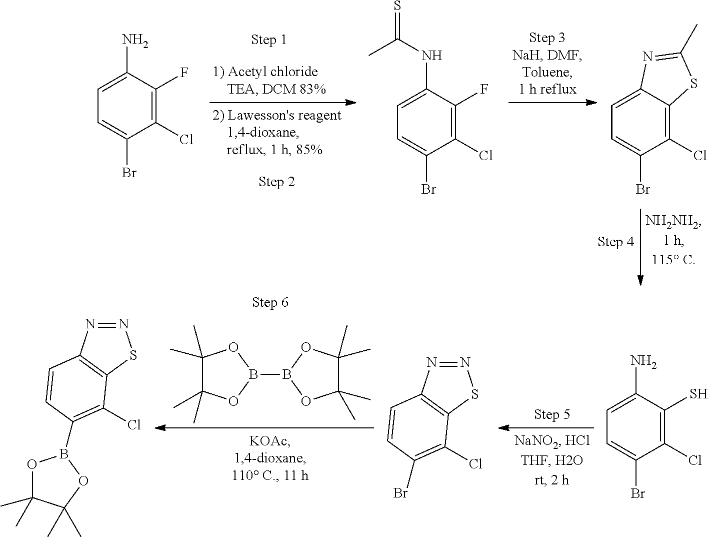

### Step 1: N-(4-bromo-3-chloro-2-fluorophenyl)acetamide

To a solution of 4-bromo-3-chloro-2-fluoroaniline (200 mg, 0.89 mmol) in DCM (2 mL) cooled to 0° C. was added AcCl (0.13 mL, 1.78 mmol) followed by TEA (0.25 mL, 1.78 mmol). The reaction mixture was stirred at rt for 1 h, then was diluted with DCM and washed with 5% citric acid sol. and NaHCO₃ sat. sol. The organic layer was dried over Na₂SO₄, filtered, and concentrated in vacuo, and the resulting crude was purified by flash chromatography (10-30% EtOAc in petroleum ether) to obtain the title compound as white crystals (196 mg, 83%). LCMS (ES⁺) Method 1A: m/z 266, 268 (M+H)⁺, RT 1.77 min.

### Step 2: N-(4-bromo-3-chloro-2-fluorophenyl)ethanethioamide

N-(4-bromo-3-chloro-2-fluorophenyl)acetamide (145 mg, 0.54 mmol) was dissolved in 1,4-dioxane (3 mL), Lawesson's reagent (187 mg, 0.46 mmol) was added and the reaction mixture was stirred at 90° C. for 45 min. The reaction was cooled to rt, diluted with EtOAc and washed with NaHCO₃ sat. sol. and brine. The organic layer was dried over Na₂SO₄, filtered, and concentrated in vacuo, and the resulting crude was purified by flash chromatography (0-20% EtOAc in petroleum ether) to obtain the title compound as beige crystals (130 mg, 85%). LCMS (ES⁺) Method 1A: m/z 282, 284 (M+H)⁺, RT 1.97 min.

### Step 3: 6-bromo-7-chloro-2-methylbenzo[d]thiazole

N-(4-bromo-3-chloro-2-fluorophenyl)ethanethioamide (128 mg, 0.45 mmol) was suspended in DMF (0.25 mL) and cooled to 0° C. NaH (60% dispersion on mineral oil, 22 mg, 0.54 mmol) was added and the reaction mixture was stirred at rt for 30 min then at 110° C. for additional 30 min (a precipitate was formed). Toluene (3 mL) was added to the hot mixture and the resulting solution was stirred at 120° C. for 45 min. The reaction was cooled to rt then H₂O and EtOAc were added. The organic layer was washed with brine, dried over Na₂SO₄, filtered, and concentrated under vacuo to obtain the title compound, as red crystals, which was used in the next step without further purification (124 mg). LCMS (ES⁺) Method 1A: m/z 262, 264 (M+H)⁺, RT 2.21 min.

### Step 4: 6-amino-3-bromo-2-chlorobenzenethiol

A mixture of 6-bromo-7-chloro-2-methylbenzo[d]thiazole (121 mg, 0.46 mmol) and N₂H4-H₂O (0.75 mL, 15.2 mmol) was stirred at 115° C. for 1.5 h. The reaction was cooled to rt, neutralized to pH 7 with HCl (37%) and extracted twice with DCM. The organic phases were combined, dried over Na₂SO₄, filtered, and concentrated in vacuo to obtain the title compound as yellow crystals, that was used in the next step without any further purification (106 mg, 96%). LCMS (ES⁺) Method 1A: m/z 238, 240 (M+H)⁺, RT 2.00 min.

### Step 5: 6-bromo-7-chlorobenzo[d][1,2,3]thiadiazole

6-Amino-3-bromo-2-chlorobenzenethiol (106 mg, 0.44 mmol) was suspended with THF (0.5 mL), then 12N HCl (0.2 mL, 2.6 mmol), NaNO₂ (40 mg, 0.58 mmol) and H₂O were sequentially added. The mixture was stirred at rt for 2 h. The reaction was then basified with K₂CO₃ sat. sol. and extracted twice with DCM. The organic phases were combined, dried over Na₂SO₄, filtered, and concentrated in vacuo. The resulting crude was purified by flash chromatography (0-2% EtOAc in petroleum ether) to obtain the title compound as light-yellow solid (60 mg, 54%). ¹H NMR (300 MHz, CDCl₃) δ 8.30 (d, J=8.8 Hz, 1H), 7.82 (d, J=8.8 Hz, 1H). LCMS (ES⁺) Method 1A, RT 2.18 min.

### Step 6: 2-(4-chloro-5-(4,4,5,5-tetramethyl-1,3,2-dioxaborolan-2-yl)-2H-indazol-2-yl)acetonitrile

The above intermediate was prepared according to the procedure described for Intermediate 15 starting from 6-bromo-7-chlorobenzo[d][1,2,3]thiadiazole and used in the next step without further purification. LCMS (ES⁺) Method 1A, RT 2.52 min.

### Intermediate 144: 4-chloro-1-methyl-5-(4,4,5,5-tetramethyl-1,3,2-dioxaborolan-2-yl)-1H-indazole

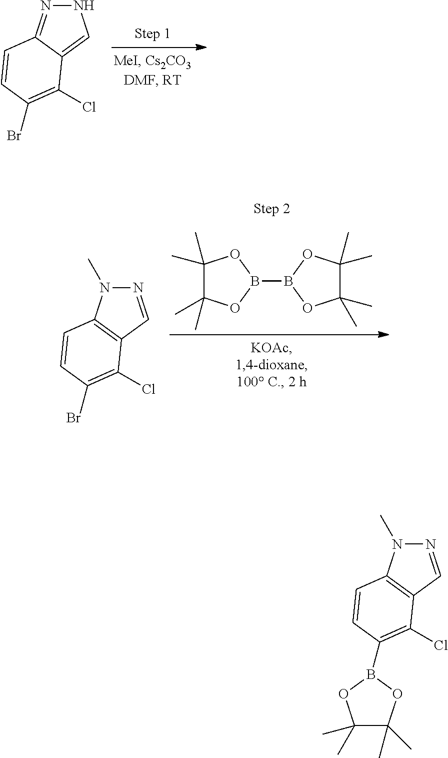

### Step 1: 5-bromo-4-chloro-1-methyl-1H-indazole

A suspension of 5-bromo-4-chloro-1H-indazole (50 mg, 0.22 mmol), Mel (0.01 mL, 0.24 mmol) and Cs₂CO₃ (141 mg, 0.43 mmol) in DMF (0.6 mL) was stirred at rt for 4.5 h. Additional Mel (0.005 mL, 0.079 mmol) was added, and the reaction mixture was stirred at rt for 1 h. The mixture was diluted with EtOAc and washed with H₂O (×2) and brine. The organic layer was dried over Na₂SO₄, filtered, and concentrated in vacuo. The resulting crude was purified by flash chromatography (10-50% EtOAc in petroleum ether) to obtain the title compound as off-white solid (36 mg, 68%). LCMS (ES⁺) Method 1A: m/z 245, 247 (M+H)⁺, RT 2.03 min.

### Step 2: 4-chloro-1-methyl-5-(4,4,5,5-tetramethyl-1,3,2-dioxaborolan-2-yl)-1H-indazole

The above intermediate was prepared according to the procedure described for Intermediate 15 starting from 5-bromo-4-chloro-1-methyl-1H-indazole and used in the next step without further purification. LCMS (ES⁺) Method 1A: m/z 293 (M+H)⁺, RT 2.19 min.

### Intermediate 145: tert-butyl (((1R,5S,6r)-3-(3-iodo-1-(tetrahydro-2H-pyran-2-yl)-1H-pyrazolo[3,4-b]pyrazin-6-yl)-6-(4-methylthiazol-2-yl)-3-azabicyclo[3.1.0]hexan-6-yl)methyl)(methyl)carbamate

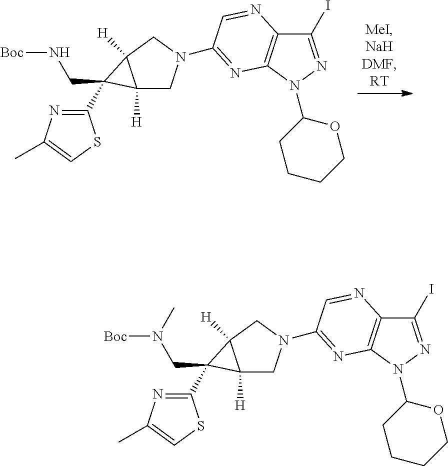 [SMILES: Cc1csc([C@@]2(CNC(=O)OC(C)(C)C)[C@@H]3CN(c4cnc5c(I)nn(C6CCCCO6)c5n4)C[C@@H]32)n1 ; Cc1csc([C@@]2(CN(C)C(=O)OC(C)(C)C)[C@@H]3CN(c4cnc5c(I)nn(C6CCCCO6)c5n4)C[C@@H]32)n1]

Intermediate 123 (20 mg, 0.03 mmol) was dissolved in DMF (0.5 mL), NaH (60% dispersion on mineral oil, 1.5 mg, 0.06 mmol) was added at rt, and after 15 min Mel (2 μL, 0.035 mmol) was added. The mixture was stirred at rt for 1 h, then additional NaH (60% dispersion on mineral oil, 2 mg, 0.08 mmol) and Mel (2 μL, 0.028 mmol) were added until reaction completion. The reaction was quenched by addition of H₂O and extracted with EtOAc. The organic layer was washed with H₂O (×2) and brine, dried over Na₂SO₄, filtered, and concentrated in vacuo to obtain the title compound, that was used in the next step without further purification (24 mg). LCMS (ES⁺) Method 1A: m/z 652 (M+H)⁺, RT 2.24 min.

### Intermediate 146: benzyl (((1R,5S,6r)-6-(5-methyl-1,3,4-thiadiazol-2-yl)-3-azabicyclo[3.1.0]hexan-6-yl)methyl)carbamate hydrochloride

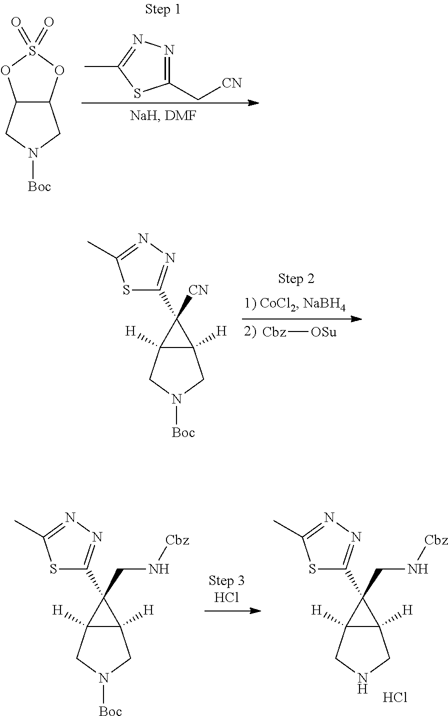

### Step 1: tert-butyl (1R,5S,6r)-6-cyano-6-(5-methyl-1,3,4-thiadiazol-2-yl)-3-azabicyclo[3.1.0]hexane-3-carboxylate

2-(5-Methyl-1,3,4-thiadiazol-2-yl)acetonitrile (115 mg, 0.83 mmol) and tert-butyl tetrahydro-5H-[1,3,2]dioxathiolo[4,5-c]pyrrole-5-carboxylate 2,2-dioxide (200 mg, 0.75 mmol) were dissolved in dry DMF (2 mL) and the resulting solution was added dropwise over a period of 30 min to a suspension of NaH (60% dispersion in mineral oil, 151 mg, 3.77 mmol) in dry DMF (1.2 mL) at rt under N₂ flow. The reaction mixture was stirred at rt for 2 h. Additional NaH (60% dispersion in mineral oil, 60 mg, 1.51 mmol) was added and after 2 h stirring at rt NaH (60% dispersion in mineral oil 30 mg, 0.76 mmol) was added again. The reaction mixture was stirred at rt for another 2 h, then was quenched by a slowly addition of H₂O and diluted with EtOAc. The organic layer was washed with H₂O (×2), dried over Na₂SO₄, filtered, and concentrated in vacuo. The resulting crude was purified by flash chromatography (40-70% EtOAc in petroleum ether) to obtain the title compound as off-white solid (55 mg, 24%). LCMS (ES⁺) Method 1A: m/z 307 (M+H)⁺, RT 1.59 min.

### Step 2: tert-butyl (1R,5S,6r)-6-((((benzyloxy)carbonyl)amino)methyl)-6-(5-methyl-1,3,4-thiadiazol-2-yl)-3-azabicyclo[3.1.0]hexane-3-carboxylate

Tert-butyl (1R,5S,6r)-6-cyano-6-(5-methyl-1,3,4-thiadiazol-2-yl)-3-azabicyclo[3.1.0]hexane-3-carboxylate (55 mg, 0.18 mmol) and CoCl₂ (8.5 mg, 0.04 mmol) were dissolved in MeOH (1.5 mL) in a pressure flask, and the dark solution was cooled to 0° C. NaBH₄ (48 mg, 1.26 mmol) was added portion wise, the flask was sealed, and the reaction mixture was stirred at 0° C. for 15 min then at rt for 1 h. Additional CoCl₂ (94 mg, 0.4 mmol) and NaBH₄ (184 mg, 4.9 mmol) were added portionwise over 4 h. The reaction mixture was then diluted with MeOH and filtered over a pad of celite. Solvent was removed in vacuo, then the residue was taken up with DCM (7 mL) and the mixture was cooled to 0° C. TEA (0.1 mL, 0.72 mmol) and N-(benzyloxycarbonyloxy)succinimide (49 mg, 0.20 mmol) were added, and the reaction was stirred at 0° C. for 30 min, then allowed to warm to rt overnight. The mixture was diluted with DCM and H₂O and the organic layer was washed with 5% citric acid sol., NaHCO₃ sat. sol., dried over Na₂SO₄, filtered, and concentrated in vacuo. The resulting crude was purified by flash chromatography (0-50% EtOAc in DCM) to obtain the title compound as colourless oil (13 mg, 16%). Method 1A: m/z 445 (M+H)⁺, RT 1.91 min.

### Step 3: benzyl (((1R,5S,6r)-6-(5-methyl-1,3,4-thiadiazol-2-yl)-3-azabicyclo[3.1.0]hexan-6-yl)methyl)carbamate hydrochloride

Tert-butyl (1R,5S,6r)-6-((((benzyloxy)carbonyl)amino)methyl)-6-(5-methyl-1,3,4-thiadiazol-2-yl)-3-azabicyclo[3.1.0]hexane-3-carboxylate (13 mg, 0.03 mmol) was dissolved in DCM (0.5 mL), HCl (4N in 1,4-dioxane, 0.2 mL, 0.8 mmol) was added and the solution was stirred at rt for 50 min. Volatiles were removed in vacuo to afford the title compound as white solid, that was used in the next step without further purification (13 mg). Method 1A: m/z 345 (M+H)⁺, RT 1.07 min.

### Intermediate 147: 4-chloro-1-(tetrahydro-2H-pyran-2-yl)-5-(4,4,5,5-tetramethyl-1,3,2-dioxaborolan-2-yl)-1H-indazole

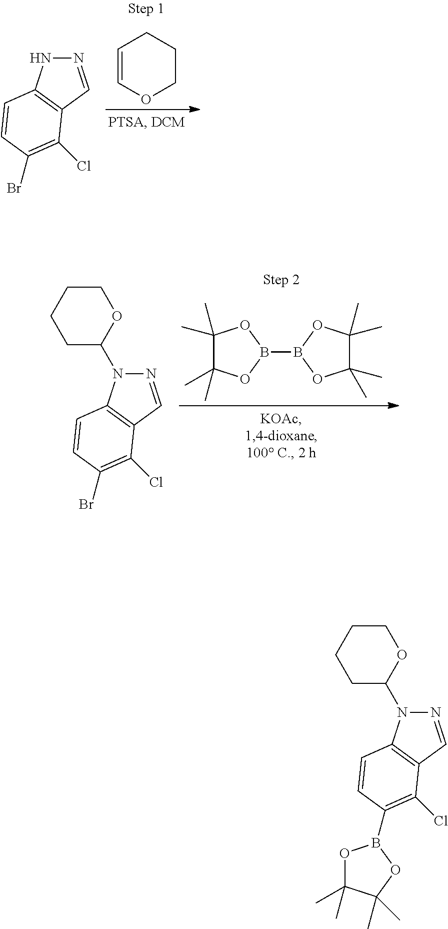

### Step 1: 5-bromo-4-chloro-1-(tetrahydro-2H-pyran-2-yl)-1H-indazole

To a suspension of 5-bromo-4-chloro-1H-indazole (33 mg, 0.14 mmol) and DHP (0.04 mL, 0.43 mmol) in DCM (0.8 mL), PTSA (2.7 mg, 0.01 mmol) was added. The reaction mixture was stirred at rt overnight, then was diluted with DCM and NaHCO₃ sat. sol. was added. The organic layer was dried over Na₂SO₄, filtered, and concentrated in vacuo. The resulting crude was purified by flash chromatography (0-10% EtOAc in petroleum ether) to obtain the title compound as white solid (41 mg, 91%). Method 1A: m/z 315, 317 (M+H)⁺, RT 2.34 min.

### Step 2: 4-chloro-1-(tetrahydro-2H-pyran-2-yl)-5-(4,4,5,5-tetramethyl-1,3,2-dioxaborolan-2-yl)-1H-indazole

The above intermediate was prepared according to the procedure described for Intermediate 15 starting from 5-bromo-4-chloro-1-(tetrahydro-2H-pyran-2-yl)-1H-indazole and used in the next step without further purification. LCMS (ES⁺) Method 1A: m/z 363 (M+H)⁺, RT 2.43 min.

### Intermediate 148: 1-(3-chloro-4-(4,4,5,5-tetramethyl-1,3,2-dioxaborolan-2-yl)phenyl)-1H-pyrazole

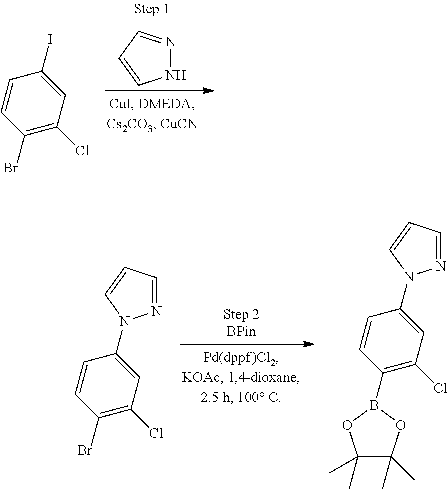 [SMILES: Clc1cc(I)ccc1Br ; c1cn[nH]c1 ; Clc1cc(-n2cccn2)ccc1Br ; CC1(C)OB(c2ccc(-n3cccn3)cc2Cl)OC1(C)C]

### Step 1: 1-(4-bromo-3-chlorophenyl)-1H-pyrazole

A mixture of 1-bromo-2-chloro-4-iodobenzene (150 mg, 0.47 mmol), 1H-pyrazole (34 mg, 0.5 mmol), CuI (18 mg, 0.09 mmol), DMEDA (0.02 mL, 0.19 mmol) and Cs₂CO₃ (308 mg, 0.95 mmol) in degassed MeCN (2 mL) was stirred at 80° C. for 2.5 h. After cooling to 25° C., the mixture was filtered, and the filtrate was concentrated under reduced pressure. The residue was purified by flash chromatography (0-10% EtOAc in petroleum ether) to yield the title compound as beige solid (105 mg, 86%). Method 1A: m/z 257, 259 (M+H)⁺, RT 2.16 min.

### Step 2: 1-(3-chloro-4-(4,4,5,5-tetramethyl-1,3,2-dioxaborolan-2-yl)phenyl)-1H-pyrazole

The above intermediate was prepared according to the procedure described for intermediate 15 starting from 1-(4-bromo-3-chlorophenyl)-1H-pyrazole and used in the next step without further purification. LCMS (ES⁺) Method 1A: m/z 305 (M+H)⁺, RT 2.32 min.

### Intermediate 149: 3-fluoro-7-iodo-N-methyl-5H-pyrrolo[2,3-b]pyrazine-5-sulfonamide

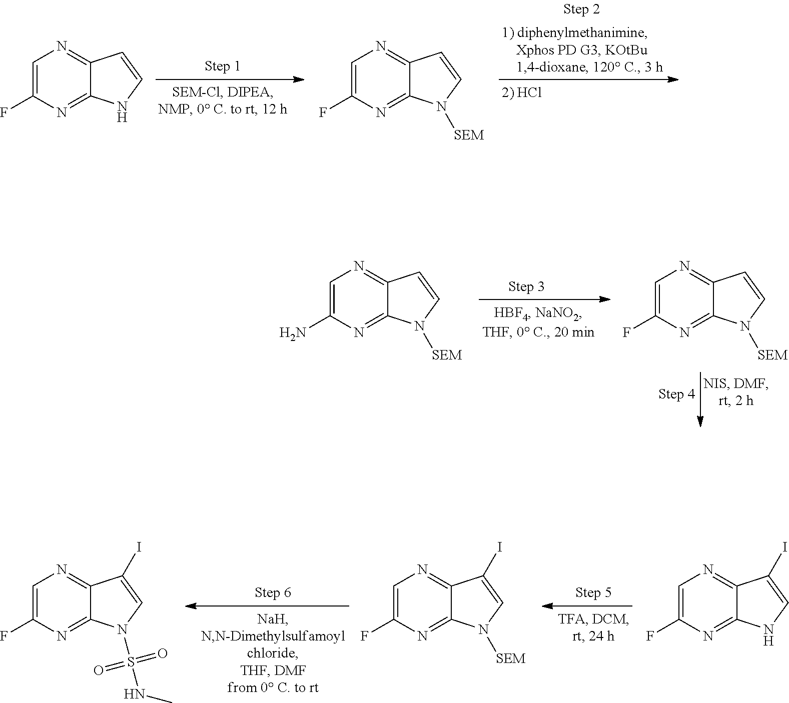

### Step 1: 3-chloro-5-((2-(trimethylsilyl)ethoxy)methyl)-5H-pyrrolo[2,3-b]pyrazine

To a solution of 3-chloro-5H-pyrrolo[2,3-b]pyrazine (1 g, 6.5 mmol) and DIPEA (1.8 mL, 10.5 mmol) in NMP (5 mL), cooled to −5° C., SEM-C₁ (1.4 mL, 7.8 mmol) in NMP (2 mL) was added over a period of 5-10 min. The mixture was warmed to rt and stirred for 12 h at rt. A 5% aq. LiCl sol. (5 mL) and EtOAc (20 mL) were added. The organic layer was washed with further 5% aq. LiCl solution (3×), 0.5 M aq. HCl, Na₂CO₃ sat. sol. and brine (100 mL). The organic layer was dried over Na₂SO₄, filtered and concentrated. The crude was purified by flash chromatography (0-50% EtOAc in petroleum ether) to afford the title compound as white solid (1.67 g, 91%). LCMS (ES⁺) Method 1A: m/z 284 (M+H)⁺, RT 2.37 min.

### Step 2: 5-((2-(trimethylsilyl)ethoxy)methyl)-5H-pyrrolo[2,3-b]pyrazin-3-amine

To a solution of 3-chloro-5-((2-(trimethylsilyl)ethoxy)methyl)-5H-pyrrolo[2,3-b]pyrazine (200 mg, 0.7 mmol), diphenylmethanimine (0.14 mL, 0.8 mmol) in 1,4-dioxane (1.5 mL), Xphos Pd G3 (26.54 mg, 0.03 mmol) and tBuOK (118.6 mg, 1.06 mmol) were sequentially added. The resulting mixture was stirred for 3 h at 120° C., and 2M HCl (5 mL) was added, and the resulting mixture was stirred at rt for 30 min. The mixture was treated with 2M NaOH until pH 10. The aqueous layer was extracted with EtOAc (3×). The collected organic layers were washed with brine, dried over Na₂SO₄, filtered, and concentrated in vacuo. The residue was purified by flash chromatography (0-50% EtOAc in petroleum ether) to afford title compound as yellow solid (34 mg, 18%). LCMS (ES⁺) Method 1A: m/z 265 (M+H)⁺, RT 1.69 min.

### Step 3: 3-fluoro-5-((2-(trimethylsilyl)ethoxy)methyl)-5H-pyrrolo[2,3-b]pyrazine

A solution of NaNO₂ (73 mg, 1.04 mmol) in H₂O (0.2 mL) was added dropwise to a solution of 5-((2-(trimethylsilyl)ethoxy)methyl)-5H-pyrrolo[2,3-b]pyrazin-3-amine (250 mg, 0.95 mmol) and HBF₄ (48% in H₂O, 1.25 mL) in THF (1.25 mL) cooled to 0° C. The resulting mixture was stirred at the same temperature for 20 min. The mixture was then diluted with NaHCO₃ sat. sol. and EtOAc; the two phases were separated, and the organic layer was washed with brine, dried over Na₂SO₄ filtered and concentrated in vacuo. The crude was purified by flash chromatography (0-50% EtOAc in petroleum ether) to afford title compound as yellow solid (80 mg, 31%). LCMS (ES⁺) Method 1A: m/z 268 (M+H)⁺, RT 2.26 min.

### Step 4: 3-fluoro-7-iodo-5-((2-(trimethylsilyl)ethoxy)methyl)-5H-pyrrolo[2,3-b]pyrazine

NIS (94 mg, 0.42 mmol) was added to a solution of 3-fluoro-5-((2-(trimethylsilyl)ethoxy)methyl)-5H-pyrrolo[2,3-b]pyrazine (80 mg, 0.3 mmol) in dry DMF (1 mL) at rt and the resulting mixture was stirred at rt for 1.5 h. The mixture was treated with a solution of Na₂S₂O₃ and ice H₂O and extracted with EtOAc (3×). The organic layer was washed with brine, dried over Na₂SO₄ filtered and concentrated in vacuo to afford the title compound, that was used in the next step without further purification. LCMS (ES⁺) Method 1A: m/z 394 (M+H)⁺, RT 2.49 min.

### Step 5: 3-fluoro-7-iodo-5H-pyrrolo[2,3-b]pyrazine

A solution of 3-fluoro-7-iodo-5-((2-(trimethylsilyl)ethoxy)methyl)-5H-pyrrolo[2,3-b]pyrazine (117 mg, 0.3 mmol) in DCM (1.2 mL) and TFA (0.8 mL) was stirred at rt for 24 h. The mixture was concentrated, and the residue was dissolved in MeOH/NH₄OH and stirred for 1 h. Volatiles were evaporated, and the resulting title compound was used in the next step without further purification. LCMS (ES⁺) Method 1A: m/z 264 (M+H)⁺, RT 1.57 min.

### Step 6: 3-fluoro-7-iodo-N,N-dimethyl-5H-pyrrolo[2,3-b]pyrazine-5-sulfonamide NaH (60% dispersion on mineral oil, 9.88 mg, 0.250 mmol) was added to a solution of 3-fluoro-7-iodo-5H-pyrrolo[2,3-b]pyrazine (50 mg, 0.19 mmol) in dry DMF/THF (1:1; 1 ml) cooled to 0° C., and the resulting mixture was stirred at rt. The mixture was cooled to 0° C., N,N-dimethylsulfamoyl chloride (0.02 mL, 0.19 mmol) was added, the mixture was warmed to rt and stirred for 24 h. NH₄Cl sat. sol. was added and the aqueous layer was extracted with EtOAc (3×). The collected organic layers were washed with brine, dried over Na₂SO₄ filtered and concentrated in vacuo. The crude was purified by flash chromatography (0-60% EtOAc in petroleum ether) to afford the title compound as white solid (25 mg, 35%). LCMS (ES⁺) Method 1A: m/z 371 (M+H)⁺, RT 1.96 min.

### Intermediate 153: benzyl (((1R,5S,6r)-6-(5-fluorothiazol-2-yl)-3-azabicyclo[3.1.0]hexan-6-yl)methyl)carbamate hydrochloride

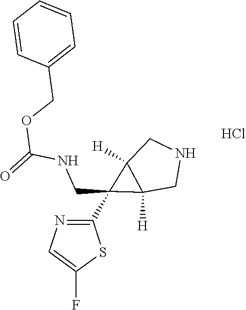 [SMILES: O=C(NC[C@]1(c2ncc(F)s2)[C@@H]2CNC[C@@H]21)OCc1ccccc1 ; Cl]

The title compound was synthesized according to the procedure reported for Intermediate 128 starting from 2-chloro-5-fluorothiazole and using sodium bis(trimethylsilyl)amide and obtained as a white solid. LCMS (ES⁺) Method 1A: m/z 348 (M+H)⁺, RT 1.27 min.

### Intermediate 155: 4-(difluoromethyl)-2-methyl-5-(4,4,5,5-tetramethyl-1,3,2-dioxaborolan-2-yl)-2H-indazole

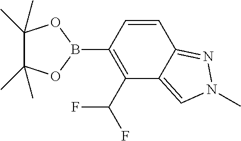

### Step 1: (2-methyl-3-nitrophenyl)methanol

A solution of 2-methyl-3-nitro-benzoic acid (1.10 g, 6.07 mmol) in THF (11 mL) at rt was treated with CDI (1.48 g, 9.11 mmol) and the mixture was stirred for 1 h. The reaction mixture was cooled to 0° C. and a solution of NaBH₄ (1.15 g, 30.36 mmol) in H₂O (11 mL) was added dropwise. The mixture was stirred at rt for 2 h. The reaction mixture was cooled at 0° C. and HCl 2M (5 mL) was added dropwise and resulting mixture stirred for 30 min at rt before being poured into NaHCO₃ sat. sol. (10 mL) and extracted with EtOAc (3×40 mL). The organic layer was washed with H₂O, brine, dried over Na₂SO₄ filtered and concentrated under reduced pressure. The crude was purified by flash chromatography (0-50% EtOAc in petroleum ether) to afford the title compound as white solid (960 mg, 95%). ¹H NMR (400 MHz, CDCl₃) δ 7.75 (d, J=8.1 Hz, 1H), 7.68 (d, J=7.5 Hz, 1H), 7.36 (t, J=7.8 Hz, 1H), 4.82 (br s, 2H), 2.48 (s, 3H), 1.74 (br s, 1H).

### Step 2: 2-methyl-3-nitrobenzaldehyde

A solution of (2-methyl-3-nitrophenyl)methanol (0.96 g, 5.74 mmol) in CHCl₃ (11 mL) at rt was treated with MnO₂ (3.49 g, 40.2 mmol). The resulting slurry was stirred for 3 h at reflux. After cooling the reaction mixture was filtered on a pad of celite, washed with DCM and concentrated in vacuo to get the title compound as a white powder (790 mg, 83%). ¹H NMR (400 MHz, CDCl₃) δ 10.41 (s, 1H), 8.07 (d, J=7.5 Hz, 1H), 8.00 (d, J=8.1 Hz, 1H), 7.55 (t, J=7.9 Hz, 1H), 2.81 (s, 3H).

### Step 3: 1-(difluoromethyl)-2-methyl-3-nitrobenzene

A solution of 2-methyl-3-nitrobenzaldehyde (450.0 mg, 2.72 mmol) in CHCl₃ (9 mL) cooled to −78° C. was treated with DAST (0.9 mL, 6.81 mmol) and stirred at rt for 16 h. H₂O was added, and the reaction mixture was extracted with DCM. The organic phase was washed with brine, dried over Na₂SO₄, filtered, and concentrated in vacuo. The crude was purified by flash chromatography (0-5% EtOAc in petroleum ether) to afford title compound as a yellow oil (490 mg, 96%). ¹H NMR (400 MHz, CDCl₃) δ 7.82 (d, J=8.1 Hz, 1H), 7.71 (d, J=7.7 Hz, 1H), 7.38 (t, J=8.0 Hz, 1H), 6.91-6.63 (m, 1H), 2.47 (s, 3H).

### Step 4: 3-(difluoromethyl)-2-methylaniline

A solution of 1-(difluoromethyl)-2-methyl-3-nitrobenzene (490.0 mg, 2.62 mmol) in EtOH (11 mL) and H₂O (5.5 mL) was treated with NH₄C₁ (70.0 mg, 1.31 mmol) and Fe (731.1 mg, 13.09 mmol) at rt and the reaction mixture was stirred at 90° C. for 2 h. After cooling the reaction mixture was filtered on a pad of solka floc, rinsed with EtOAc and concentrated under reduced pressure. The residue was taken up in EtOAc and washed with brine, dried over Na₂SO₄, filtered, and concentrated in vacuo to get the title compound (410 mg, 99%) which was used without further purification. LCMS (ES⁺) Method 2: m/z 158 (M+H)⁺, RT 0.73 min.

### Step 5: 4-bromo-3-(difluoromethyl)-2-methylaniline

A solution of 3-(difluoromethyl)-2-methylaniline (410.0 mg, 2.61 mmol) in DMF (11 mL) at 0° C. was treated with NBS (557.2 mg, 3.13 mmol) and the mixture was stirred at rt for 3 h before being poured into ice and extracted with EtOAc (3×30 mL). The combined organic layers were washed with brine, dried over Na₂SO₄, filtered, and concentrated in vacuo. The crude was purified by flash chromatography (0-20% EtOAc in petroleum ether) to afford title compound as a yellow oil (460 mg, 75%). LCMS (ES⁺) Method 2: m/z 236 (M+H)⁺, RT 1.47 min.

### Step 6: 5-bromo-4-(difluoromethyl)-2H-indazole

A solution of NaNO₂ (168.1 mg, 2.44 mmol) in H₂O (0.4 mL) was added dropwise to an ice bath cooled solution of 4-bromo-3-(difluoromethyl)-2-methylaniline (0.46 g, 1.95 mmol) in AcOH (8.5 mL). The resulting mixture was stirred for 1 h at rt. The solvent was concentrated under reduced pressure and the residue was diluted with EtOAc and washed with NaHCO₃ sat. sol. (3×20 mL) and brine. The organic phase was dried over Na₂SO₄, filtered, and concentrated in vacuo. The crude was purified by flash chromatography (0-40% EtOAc in petroleum ether) to afford the title compound as a yellow solid (358 mg, 74%). LCMS (ES⁺) Method 2: m/z 247 (M+H)⁺, RT 1.49 min.

### Step 7: 5-bromo-4-(difluoromethyl)-2-methyl-2H-indazole

A solution of 5-bromo-4-(difluoromethyl)-2H-indazole (0.36 g, 1.45 mmol) in EtOAc (4 mL) was treated with trimethyloxonium tetrafluoroborate (235.8 mg, 1.59 mmol) and the mixture was stirred at rt for 4 h before being quenched with NaHCO₃ sat. sol. and extracted with EtOAc. The organic layer was washed with brine, dried over Na₂SO₄, filtered, and concentrated in vacuo. The crude was purified by flash chromatography (0-40% EtOAc in petroleum ether) to afford title compound as a red solid (340 mg, 90%). LCMS (ES⁺) Method 2: m/z 261 (M+H)⁺, RT 1.61 min.

### Step 8: 4-(difluoromethyl)-2-methyl-5-(4,4,5,5-tetramethyl-1,3,2-dioxaborolan-2-yl)-2H-indazole

A solution of Pd(dppf)Cl₂-DCM (107.7 mg, 0.13 mmol), 5-bromo-4-(difluoromethyl)-2-methyl-2H-indazole (340.0 mg, 1.3 mmol), B₂pin₂ (529.1 mg, 2.08 mmol) and KOAc (319.5 mg, 3.26 mmol) in degassed 1,4-dioxane (4.7 mL) was heated in a sealed vial at 90° C. for 3 h. After cooling the reaction mixture was filtered on a pad of solka-floc and concentrated under reduced pressure to get the title compound (401 mg, 99%) which was used without further purification. Method 2: m/z 309 (M+H)⁺, RT 1.96 min

### Intermediate 156: 1,4-dimethyl-5-(4,4,5,5-tetramethyl-1,3,2-dioxaborolan-2-yl)-1H-benzo[d][1,2,3]triazole

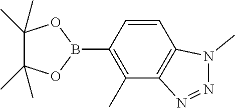

### Step 1: N, 3-dimethyl-2-nitroaniline

A solution of 1-fluoro-3-methyl-2-nitrobenzene (20.0 g, 128.9 mmol) in EtOH (99 mL) was treated with methanamine (40% in H₂O; 39.0 mL, 451.2 mmol) and stirred at reflux for 18 h. The reaction mixture was cooled at rt affording a precipitate, which was isolated by filtration, to get the title compound (21.1 g, 98%) as an orange solid. LCMS (ES⁺) Method 2: m/z 167 (M+H)⁺, RT 1.80 min.

### Step 2: 4-bromo-N,3-dimethyl-2-nitroaniline

A solution of N,3-dimethyl-2-nitroaniline (10.0 g, 60.18 mmol) in DMF (60 mL) was cooled to 0° C. and treated with a solution of NBS (10.7 g, 60.18 mmol) in DMF (50 mL). The reaction mixture was stirred for 2 h. H₂O (250 mL) was added, and the precipitate formed was filtered to afford the title compound (14.3 g, 97%) as an orange powder. LCMS (ES⁺) Method 2: m/z 245, 247 (M+H)⁺, RT 2.08 min.

### Step 3: 4-bromo-N1,3-dimethylbenzene-1,2-diamine

A solution of 4-bromo-N,3-dimethyl-2-nitroaniline (14.3 g, 58.35 mmol) in AcOH/EtOH (1/1; 180 ml) at 0° C. was treated with Fe (16.3 g, 291.75 mmol) followed by HCl (2 N aq. sol.; 58.4 mL, 116.7 mmol). The reaction mixture was stirred for 1 h, filtered through a pad of celite, and the filtrate concentrated to remove volatiles. The residue was diluted with EtOAc (160 ml) and H₂O (160 ml) and the layers were separated. The organic phase was washed with H₂O (100 ml), NaHCO₃ sat. sol., brine and evaporated under reduced pressure to afford the title compound (10.9 g, 87%) as a brown oil. LCMS (ES⁺) Method 2: m/z 215 (M+H)⁺, RT 1.20 min.

### Step 4: 5-bromo-1,4-dimethyl-1H-benzo[d][1,2,3]triazole

A solution of tert-butyl nitrite (6.0 ml) in MeCN (10 mL) at 0° C. was treated with HBF₄ (48% in H₂O; 10 mL) followed by a solution of 4-bromo-N1,3-dimethylbenzene-1,2-diamine (7.0 g, 32.54 mmol) in MeCN (50 mL). The reaction mixture was stirred at 5° C. for 2 h and then at rt for 1 h before poured into a stirred solution of NaOH (10.4 g, 260.3 mmol) in H₂O (300 mL). The solid formed was isolated by filtration and washed with H₂O and purified by flash chromatography (10-100% EtOAc in petroleum ether) to obtain the title compound as a pale brown solid (5 g, 68%). LCMS (ES⁺) Method 2: m/z 226 (M+H)⁺, RT 1.56 min.

### Step 5: 1,4-dimethyl-5-(4,4,5,5-tetramethyl-1,3,2-dioxaborolan-2-yl)-1H-benzo[d][1,2,3]triazole

The title compound was prepared as reported in the synthesis of Intermediate 155 step 8. LCMS (ES⁺) Method 2: m/z 274 (M+H)⁺, RT 1.90 min.

### Intermediate 157: 2,4-dimethyl-5-(4,4,5,5-tetramethyl-1,3,2-dioxaborolan-2-yl)-2H-benzo[d][1,2,3]triazole

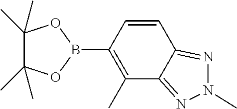

### Step 1: 4-bromo-3-methyl-2-nitroaniline

A solution of 3-methyl-2-nitroaniline (5.0 g, 32.86 mmol) and NBS (5.85 g, 32.86 mmol) in AcOH (50 mL) was heated at 110° C. for 1 h. After cooling, H₂O was added dropwise, and the suspension was stirred at 5° C. for 1 h. The solid formed was filtered to afford the title compound (6.4 g, 84%) as an orange powder. LCMS (ES⁺) Method 2: m/z 231 (M+H)⁺, RT 1.78 min.

### Step 2: 4-bromo-3-methylbenzene-1,2-diamine

A solution of 4-bromo-3-methyl-2-nitroaniline (600 mg, 2.6 mmol) in EtOH (8 mL) was treated with SnCl₂ (1.97 g, 10.39 mmol) and stirred at 70° C. for 20 h before being cooled and poured into H₂O. NaOH (1 N aq. sol.) was added to the mixture to adjust pH>7. The resulting mixture was extracted with CHCl₃ (3×). The combined organics were dried over Na₂SO₄, filtered, and concentrated in vacuo to give the title compound (522 mg, 99%) as a brown solid. LCMS (ES⁺) Method 2: m/z 201 (M+H)⁺, RT 0.94 min.

### Step 3: 5-bromo-4-methyl-2H-benzo[d][1,2,3]triazole

A solution of 4-bromo-3-methylbenzene-1,2-diamine (550 mg, 2.74 mmol) in AcOH (4 mL) and H₂O (0.6 mL) at 0° C. was treated with HCl (6 M aq. sol.; 228 uL, 1.37 mmol) followed by dropwise addition of a solution of NaNO₂ (207.6 mg, 3.01 mmol) in H₂O (0.5 mL). The reaction mixture was stirred at rt for 1 h before being treated with H₂O (60 mL). The solid formed was filtered, washed with H₂O, and dried to afford the title compound (340 mg, 59%) as a red solid. LCMS (ES⁺) Method 2: m/z 212 (M+H)⁺, RT 1.38 min.

### Step 4: 5-bromo-2,4-dimethyl-2H-benzo[d][1,2,3]triazole

A solution of 5-bromo-4-methyl-2H-benzo[d][1,2,3]triazole (340 mg, 1.6 mmol) in THF (7 mL) was treated at rt with PPh₃ (546.7 mg, 2.08 mmol), MeOH (91 uL, 2.24 mmol) and DIAD (409 uL, 2.08 mmol). The resulting dark solution was stirred at rt for 2 h and then concentrated under reduced pressure and purified by flash chromatography (0-30% EtOAc in petroleum ether) to afford title compound (127 mg, 35%). LCMS (ES⁺) Method 2: m/z 226 (M+H)⁺, RT 1.78 min.

### Step 5: 2,4-dimethyl-5-(4,4,5,5-tetramethyl-1,3,2-dioxaborolan-2-yl)-2H-benzo[d][1,2,3]triazole

The title compound was prepared as reported in the synthesis of Intermediate 155 in step 8. LCMS (ES⁺) Method 2: m/z 274 (M+H)⁺, RT 2.04 min.

### Intermediate 158: 4-chloro-2-ethyl-5-(4,4,5,5-tetramethyl-1,3,2-dioxaborolan-2-yl)-2H-indazole

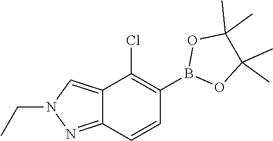 [SMILES: CCn1cc2c(Cl)c(B3OC(C)(C)C(C)(C)O3)ccc2n1]

Intermediate 158 was synthesized according to the procedure reported for Intermediate 45 using triethyloxonium hexafluorophosphate in step 2 and obtained as a white solid. LCMS (ES⁺) Method 2: m/z 307 (M+H)⁺, RT 2.05 min.

### Intermediate 159: (8-methyl-[1,2,4]triazolo[4,3-a]pyridin-7-yl)boronic acid

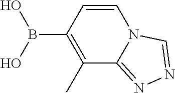

### Step 1: 4-bromo-2-hydrazineyl-3-methylpyridine

A solution of 4-bromo-2-fluoro-3-methylpyridine (1.0 g, 5.26 mmol) in EtOH (2 mL) was treated with N₂H₄·H₂O (2.56 mL, 52.63 mmol) and the mixture was stirred at rt for 24 h before being quenched with NaOH (2 M aq. sol.; 2.5 mL) and H₂O (5 mL). The solid formed was filtered and dried under reduced pressure to afford the title compound (0.26 g, 24%) as a white solid. LCMS (ES⁺) Method 2: m/z 202 (M+H)⁺, RT 0.27 min.

### Step 2: 7-bromo-8-methyl-[1,2,4]triazolo[4,3-a]pyridine

A solution of 4-bromo-2-hydrazineyl-3-methylpyridine (260.0 mg, 1.29 mmol) in formic acid (0.97 mL, 25.74 mmol) was stirred at 105° C. for 3 h in a sealed tube. After cooling the mixture was concentrated under reduced pressure and the residue taken up in EtOAc, washed with H₂O, brine, dried over Na₂SO₄, filtered, and concentrated in vacuo to give the title compound (272 mg, 99%) as a white solid. LCMS (ES⁺) Method 2: m/z 212 (M+H)⁺, RT 0.75 min.

### Step 3: (8-methyl-[1,2,4]triazolo[4,3-a]pyridin-7-yl)boronic acid

The title compound was prepared as reported in the synthesis of Intermediate 155 in step 8. LCMS (ES⁺) Method 2: m/z 178 (M+H)⁺, RT 0.27 min.

### Intermediate 160: 3-(2,3-dichloro-4-(4,4,5,5-tetramethyl-1,3,2-dioxaborolan-2-yl)phenoxy)-1-methyl-1H-pyrazole

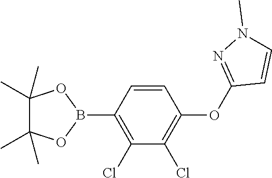 [SMILES: Cn1ccc(Oc2ccc(B3OC(C)(C)C(C)(C)O3)c(Cl)c2Cl)n1]

### Step 1: 3-(4-bromo-2,3-dichlorophenoxy)-1-methyl-1H-pyrazole

A solution of 1-methyl-1H-pyrazol-3-ol (48.27 mg, 0.49 mmol) and 1-bromo-2,3-dichloro-4-fluorobenzene (100 mg, 0.41 mmol) in DMF (2.0 mL) was treated with K₂CO₃ (113 mg, 0.82 mmol) and the mixture was stirred at 90° C. for 12 h. After cooling the mixture was diluted with EtOAc and washed with 1M HCl and brine, dried over Na₂SO₄, filtered, and concentrated in vacuo to give the title compound (114 mg, 82%) as a brown solid. LCMS (ES⁺) Method 2: m/z 321-323 (M+H)⁺, RT 2.09 min.

### Step 2: 3-(2,3-dichloro-4-(4,4,5,5-tetramethyl-1,3,2-dioxaborolan-2-yl)phenoxy)-1-methyl-1H-pyrazole

The title compound was prepared as reported in the synthesis of Intermediate 155 in step 8. LCMS (ES⁺) Method 2: m/z 369 (M+H)⁺, RT 2.28 min.

### Intermediate 161: 5-(4,4,5,5-tetramethyl-1,3,2-dioxaborolan-2-yl)quinoline-8-carboxamide

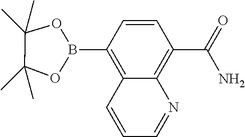

### Step 1: 5-(4,4,5,5-tetramethyl-1,3,2-dioxaborolan-2-yl)quinoline-8-carboxamide

A solution of methyl 5-(4,4,5,5-tetramethyl-1,3,2-dioxaborolan-2-yl)quinoline-8-carboxylate (50.0 mg, 0.16 mmol) in NH₃ (7 N in MeOH; 0.5 mL, 3.5 mmol) was stirred at 60° C. for 24 h before being concentrated in vacuo. The residue was purified by flash chromatography (0-20% MeOH in DCM) to afford title compound as a beige solid (41 mg, 86%). LCMS (ES⁺) Method 2: m/z 299 (M+H)⁺, RT 1.86 min.

### Intermediate 162: 5-methyl-6-(4,4,5,5-tetramethyl-1,3,2-dioxaborolan-2-yl)quinoxaline

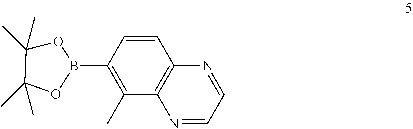

### Step 1: 6-bromo-5-methylquinoxaline

A solution of 4-bromo-3-methylbenzene-1,2-diamine (100 mg, 0.50 mmol) and oxaldehyde (0.03 mL, 0.50 mmol) in EtOH (2.5 mL) was treated with Selectfluor (1.76 mg, 0.005 mmol) and the mixture was stirred at rt for 10 min. The solvent was concentrated under reduced pressure and the residue was purified by flash chromatography (0-100% EtOAc in petroleum ether) to afford title compound as an orange solid (60 mg, 54%). LCMS (ES⁺) Method 2: m/z 223 (M+H)⁺, RT 1.82 min.

### Step 2: 5-methyl-6-(4,4,5,5-tetramethyl-1,3,2-dioxaborolan-2-yl)quinoxaline

The title compound was prepared as reported in the synthesis of Intermediate 155 in step 8. LCMS (ES⁺) Method 2: m/z 271 (M+H)⁺, RT 2.10 min.

### Intermediate 163: 4-borono-2,3-dichlorobenzoic acid

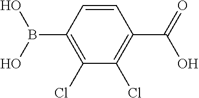 [SMILES: O=C(O)c1ccc(B(O)O)c(Cl)c1Cl]

### Step 1: 4-borono-2,3-dichlorobenzoic acid

A solution of LDA (0.21 mL, 1.57 mmol) in THF (1.6 mL) at 0° C. was treated with n-BuLi (0.79 mL, 1.57 mmol) dropwise while keeping the temperature at 0-5° C. The color of the solution turned yellow. The mixture was stirred at 0-5° C. for 30 min before being cooled to −78° C. 2,3-dichlorobenzoic acid (100 mg, 0.52 mmol) was dissolved in THF (0.48 mL) and added to the previous solution dropwise and the mixture was stirred for 30 min. Triisopropyl borate (108.3 mg, 0.58 mmol) was added dropwise to the reaction mixture which was stirred at this temperature for 1 h and at rt for 12 h before being quenched with HCl (1N aq. sol.). The resulting aqueous layer was separated and extracted with EtOAc (2×). The organic layers were washed with brine, dried over Na₂SO₄, filtered, and concentrated in vacuo to give a residue which was purified by elution on SAX cartridge (5 g; 0-100% MeOH in DCM then HCl in MeOH (3N)) to afford the title compound as a brown oil (12 mg, 10%). LCMS (ES⁺) Method 2: m/z 233 (M−H)⁻, RT 0.66 min.

### Intermediate 164: (5-chloro-1-methyl-6-oxo-1,6-dihydropyridin-2-yl)boronic acid

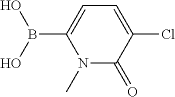

### Step 1: 6-bromo-3-chloropyridin-2(1H)-one

A solution of 6-bromo-2,3-dichloropyridine (200.0 mg, 0.88 mmol) in t-BuOH (8.8 mL) was treated with tBuOK (395.7 mg, 3.53 mmol) and the mixture was heated at 100° C. for 12 h. After cooling, HCl (1N aq. sol.) was added to the mixture which was extracted with EtOAc. The organic phase was washed with brine, dried over Na₂SO₄, filtered, and concentrated in vacuo to give a residue which was purified by flash chromatography (0-100% EtOAc in petroleum ether) to afford title compound as a white solid (85 mg, 46%). LCMS (ES⁺) Method 2: m/z 208, 210 (M+H)⁺, RT 0.97 min.

### Step 2: 6-bromo-3-chloro-1-methylpyridin-2(1H)-one

A solution of 6-bromo-3-chloropyridin-2(1H)-one (85.0 mg, 0.41 mmol) in MeCN (4.0 mL) was treated with K₂CO₃ (124 mg, 0.90 mmol) and Mel (38.1 uL, 0.61 mmol) and the reaction mixture was stirred at rt for 10 min before being quenched by addition of H₂O and EtOAc. The organic phase was dried over Na₂SO₄, filtered, and concentrated in vacuo to give a residue which was purified by flash chromatography (0-100% EtOAc in petroleum ether) to afford the title compound as a yellow solid (56 mg, 62%). LCMS (ES⁺) Method 2: m/z 222, 224 (M+H)⁺, RT 1.15 min.

### Step 3: (5-chloro-1-methyl-6-oxo-1,6-dihydropyridin-2-yl)boronic acid

The title compound was prepared as reported in the synthesis of Intermediate 155 in step 8. LCMS (ES⁺) Method 2: m/z 188 (M+H)⁺, RT 0.57 min.

### Intermediate 165: 4-chloro-1,3-dimethyl-5-(4,4,5,5-tetramethyl-1,3,2-dioxaborolan-2-yl)-1,3-dihydro-2H-benzo[d]imidazol-2-one

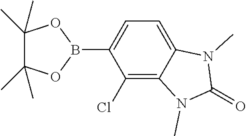 [SMILES: Cn1c(=O)n(C)c2c(Cl)c(B3OC(C)(C)C(C)(C)O3)ccc21]

### Step 1: 5-bromo-4-chloro-1,3-dihydro-2H-benzo[d]imidazol-2-one

A solution of 4-bromo-3-chlorobenzene-1,2-diamine (200.0 mg, 0.90 mmol) in DMF (3.6 mL) was treated with CDI (219.6 mg, 1.35 mmol) and stirred for 12 h at rt. H₂O was added until a precipitation was observed. The solid formed was washed with H₂O containing few drops of HCl (2N aq. sol.) and dried under vacuo to afford the title compound as a dark yellow solid (216 mg, 97%). LCMS (ES⁺) Method 2: m/z 247, 249 (M+H)⁺, RT 1.36 min.

### Step 2: 5-bromo-4-chloro-1,3-dimethyl-1,3-dihydro-2H-benzo[d]imidazol-2-one

A solution of 5-bromo-4-chloro-1,3-dihydro-2H-benzo[d]imidazol-2-one (100.0 mg, 0.40 mmol) in DMF (1.35 mL) was treated with K₂CO₃ (139.6 mg, 1.01 mmol) and Mel (0.10 mL, 1.62 mmol). The mixture was heated at 75° C. for 2 h. After cooling, the reaction mixture was diluted in H₂O and extracted with EtOAc. The organic phase was dried over Na₂SO₄, filtered, and concentrated in vacuo to afford the title compound as a yellow solid (111 mg, 99%). LCMS (ES⁺) Method 2: m/z 275, 277 (M+H)⁺, RT 1.69 min.

### Step 3: 4-chloro-1,3-dimethyl-5-(4,4,5,5-tetramethyl-1,3,2-dioxaborolan-2-yl)-1,3-dihydro-2H-benzo[d]imidazol-2-one

The title compound was prepared as reported in the synthesis of Intermediate 155 in step 8. LCMS (ES⁺) Method 2: m/z 323 (M+H)⁺, RT 1.91 min.

### Intermediate 166: 4-chloro-5-(4,4,5,5-tetramethyl-1,3,2-dioxaborolan-2-yl)-1,3-dihydro-2H-benzo[d]imidazol-2-one

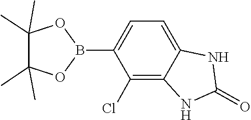 [SMILES: CC1(C)OB(c2ccc3[nH]c(=O)[nH]c3c2Cl)OC1(C)C]

The title compound was prepared as reported in the synthesis of Intermediate 155 in step 8 starting from 5-bromo-4-chloro-1,3-dihydro-2H-benzo[d]imidazol-2-one (prepared as described in the synthesis of Intermediate 165 in step 1). LCMS (ES⁺) Method 2: m/z 293 (M−H)⁻, RT 1.48 min.

### Intermediate 167: 5-chloro-4-methyl-6-(4,4,5,5-tetramethyl-1,3,2-dioxaborolan-2-yl)-3,4-dihydro-2H-benzo[b][1,4]oxazine

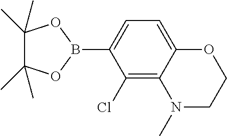 [SMILES: CN1CCOc2ccc(B3OC(C)(C)C(C)(C)O3)c(Cl)c21]

### Step 1: 6-bromo-5-chloro-3,4-dihydro-2H-benzo[b][1,4]oxazine

A solution of 6-bromo-5-chloro-2H-benzo[b][1,4]oxazin-3(4H)-one (50.0 mg, 0.19 mmol) in THF (1.5 mL) was treated with BMS (0.14 mL, 0.29 mmol) at rt and the mixture was stirred at 55° C. for 2 h before being cooled to 0° C. NaOH (1M aq. sol.; 0.5 mL) was added dropwise and the resulting mixture was stirred for 20 min at rt. H₂O (1 mL) was added and the mixture was concentrated in vacuo to get a residue which was dissolved in EtOAc and washed with brine, dried over Na₂SO₄, filtered, and concentrated in vacuo to afford the title compound (53 mg, 80% pure, 88%) which was used without further purification. LCMS (ES⁺) Method 2: m/z 248, 250 (M+H)⁺, RT 1.86 min.

### Step 2: 6-bromo-5-chloro-4-methyl-3,4-dihydro-2H-benzo[b][1,4]oxazine

A suspension of NaH (60% in mineral oil; 5.65 mg, 0.14 mmol) in DMF (0.4 mL) at 0° C. was treated with a solution of 6-bromo-5-chloro-3,4-dihydro-2H-benzo[b][1,4]oxazine (27.0 mg, 0.11 mmol) in DMF (0.4 mL). After 15 min, Mel (27 uL, 0.43 mmol) was added and the mixture was stirred at rt for 1 h before being quenched with H₂O, diluted with EtOAc and washed with H₂O, brine, dried over Na₂SO₄, filtered and concentrated in vacuo to afford a residue which was purified by flash chromatography (0-80% EtOAc in petroleum ether) to afford title compound as a colorless oil (22 mg, 77%). LCMS (ES⁺) Method 2: m/z 262, 264 (M+H)⁺, RT 2.06 min.

### Step 3: 5-chloro-4-methyl-6-(4,4,5,5-tetramethyl-1,3,2-dioxaborolan-2-yl)-3,4-dihydro-2H-benzo[b][1,4]oxazine

The title compound was prepared as reported in the synthesis of Intermediate 155 in step 8. LCMS (ES⁺) Method 2: m/z 310 (M+H)⁺, RT 2.11 min.

### Intermediate 169: 1-(3-chloro-4-(4,4,5,5-tetramethyl-1,3,2-dioxaborolan-2-yl)phenyl)pyrrolidin-2-one

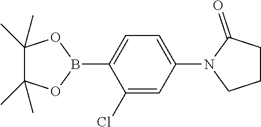

### Step 1: 1-(4-bromo-3-chlorophenyl)pyrrolidin-2-one

A solution of 4-bromo-3-chloroaniline (200.0 mg, 0.97 mmol) in THF (4.8 mL) was treated with TEA (0.14 mL, 0.97 mmol) and 4-chlorobutanoyl chloride (136.6 mg, 0.97 mmol) at 0° C. and stirred at rt for 30 min before being partitioned between EtOAc and NH₄Cl sat. sol. The organic phase was washed with brine, dried over Na₂SO₄, filtered, and concentrated in vacuo to afford a residue which was dissolved in THF (5.0 mL), treated with NaH (60% in mineral oil; 77.5 mg, 1.94 mmol) and stirred at rt for 12 h. EtOAc was added and the mixture was washed with NH₄Cl sat. aq. sol., brine, dried over Na₂SO₄, filtered, and concentrated in vacuo to give a residue which was purified by flash chromatography (0-100% EtOAc in petroleum ether) to afford the title compound as a white solid (223 mg, 84%). LCMS (ES⁺) Method 2: m/z 274, 276 (M+H)⁺, RT 1.74 min.

### Step 2: 1-(3-chloro-4-(4,4,5,5-tetramethyl-1,3,2-dioxaborolan-2-yl)phenyl)pyrrolidin-2-one

The title compound was prepared as reported in the synthesis of Intermediate 155 in step 8. LCMS (ES⁺) Method 2: m/z 322 (M+H)⁺, RT 1.96 min.

### Intermediate 170: 5-chloro-2-methyl-6-(4,4,5,5-tetramethyl-1,3,2-dioxaborolan-2-yl)phthalazin-1(2H)-one

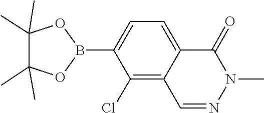

### Step 1: 4-bromo-3-chloro-N,N-diethylbenzamide

A solution of 4-bromo-3-chlorobenzoic acid (300 mg, 1.27 mmol) in a mixture of DCE (3.2 mL) and DMF (2.5 uL) at 0° C. was treated dropwise with oxalyl dichloride (1M in DCM; 2.55 mL, 2.55 mmol) and stirred at rt for 2 h before being co-evaporated with toluene. The crude was dissolved in DCE (1.5 mL) at 0° C. and treated dropwise with a solution of DEA (0.32 mL, 3.06 mmol) in DCE (1.5 mL). The mixture was stirred at rt for 18 h, the volatiles were removed under reduced pressure and the residue taken up in DCM, washed with HCl (1N aq. sol.), H₂O, and brine, dried over Na₂SO₄, filtered, and concentrated in vacuo to afford title compound (365 mg, 99%) which was used without further purification. LCMS (ES⁺) Method 2: m/z 290 (M+H)⁺, RT 1.84 min.

### Step 2: 4-bromo-3-chloro-N,N-diethyl-2-formylbenzamide

A solution of 2,2,6,6-tetramethylpiperidine (0.43 mL, 2.51 mmol) in THF (12.6 mL) at 0° C. was treated with nBuLi (2.5 M in THF; 1.06 mL, 2.64 mmol) before being cooled to −78° C. and treated with a solution of 4-bromo-3-chloro-N,N-diethylbenzamide (365.0 mg, 1.26 mmol) in THF (1.25 mL). After stirring 1 h at −78° C. DMF (0.31 mL) was added, and the mixture was stirred a further hour at −78° C. H₂O was slowly added and the mixture was washed with EtOAc (2×). Combined organic layers were washed with brine, dried over Na₂SO₄, filtered, and concentrated in vacuo to afford title compound as a yellow oil (366 mg, 85% pure; 78%) which was used without further purification. LCMS (ES⁺) Method 2: m/z 318 (M+H)⁺, RT 1.65 min.

### Step 3: 5-bromo-4-chloro-3-hydroxyisobenzofuran-1(3H)-one

A suspension of 4-bromo-3-chloro-N,N-diethyl-2-formylbenzamide (366.0 mg, 0.98 mmol) in HCl (6N aq. sol.; 4.88 mL, 29.29 mmol) was stirred at 105° C. for 18 h. After cooling the mixture was extracted with EtOAc, and the organic phase was washed with H₂O, brine, dried over Na₂SO₄, filtered, and concentrated in vacuo to afford title compound as a brown solid (258 mg, 95% pure; 95%) which was used without further purification. LCMS (ES⁺) Method 2: m/z 261-263 (M−H)⁻, RT 1.35 min.

### Step 4: 6-bromo-5-chlorophthalazin-1(2H)-one

A suspension of 5-bromo-4-chloro-3-hydroxyisobenzofuran-1(3H)-one (258.0 mg, 0.93 mmol) in IPA (2.2 mL) was stirred for 90 min at 90° C. before being treated with N₂H₄·H₂O (0.09 mL, 1.86 mmol). After cooling, a solid precipitated and filtered, washed with IPA to afford title compound as a beige powder (210 mg, 86%) which was used without further purification. LCMS (ES⁺) Method 2: m/z 259, 261 (M+H)⁺, RT 1.39 min.

### Step 5: 6-bromo-5-chloro-2-methylphthalazin-1(2H)-one

The title compound was prepared as described in the synthesis of Intermediate 165, step 2 (the reaction was performed at rt for 18 h) and obtained as a yellow solid (52 mg, 99%). LCMS (ES⁺) Method 2: m/z 273, 275 (M+H)⁺, RT 1.67 min.

### Step 6: 5-chloro-2-methyl-6-(4,4,5,5-tetramethyl-1,3,2-dioxaborolan-2-yl)phthalazin-1(2H)-one

The title compound was prepared as reported in the synthesis of Intermediate 155 in step 8. LCMS (ES⁺) Method 2: m/z 321 (M+H)⁺, RT 1.96 min.

### Intermediate 171: 4-chloro-1,2-dimethyl-5-(4,4,5,5-tetramethyl-1,3,2-dioxaborolan-2-yl)-1H-benzo[d]imidazole

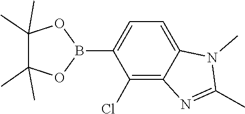

### Step 1: 6-bromo-7-chloro-2-methyl-1H-benzo[d]imidazole

A suspension of 4-bromo-3-chlorobenzene-1,2-diamine (360 mg, 1.63 mmol) in AcOH (1.39 mL) was heated at reflux for 12 h. After cooling to rt, the mixture was slowly basified to pH 9-10 with NH₄OH (30% aq. sol.). The solid formed was filtered off, washed with H₂O, and then dried in vacuo to afford the title compound as a brown solid (350 mg, 88%) which was used without further purification. LCMS (ES⁺) Method 2: m/z 245 (M+H)⁺, RT 1.00 min.

### Step 2: 5-bromo-4-chloro-1,2-dimethyl-1H-benzo[d]imidazole

A solution of 6-bromo-7-chloro-2-methyl-1H-benzo[d]imidazole (350.0 mg, 1.43 mmol) in DMF (3 mL) at 0° C. was treated with NaH (60% in mineral oil; 68.43 mg, 1.71 mmol) and the mixture was stirred for 30 min before being treated with Mel (107 uL, 1.71 mmol) and stirred at rt for 18 h. The mixture was diluted with EtOAc and poured into H₂O. The organic phase was separated and washed with brine, dried over Na₂SO₄, filtered, and concentrated in vacuo to afford a residue which was purified by preparative HPLC to obtain the title compound as a white solid (176 mg, 48%). LCMS (ES⁺) Method 2: m/z 259, 261 (M+H)⁺, RT 1.12 min.

### Step 3: 4-chloro-1,2-dimethyl-5-(4,4,5,5-tetramethyl-1,3,2-dioxaborolan-2-yl)-1H-benzo[d]imidazole

The title compound was prepared as reported in the synthesis of Intermediate 155 in step 8. LCMS (ES⁺) Method 2: m/z 307 (M+H)⁺, RT 1.27 min.

### Intermediate 172: 4-chloro-2-methyl-5-(4,4,5,5-tetramethyl-1,3,2-dioxaborolan-2-yl)isoindolin-1-one

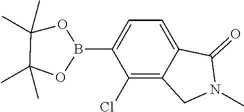

### Step 1: methyl 4-bromo-3-chloro-2-methylbenzoate

A solution of 4-bromo-3-chloro-2-methylbenzoic acid (300.0 mg, 1.2 mmol) and K₂CO₃ (332.4 mg, 2.4 mmol) in DMF (6.7 mL) was treated with Mel (0.11 mL, 1.8 mmol) and stirred at rt for 1 h before being quenched with ice cold H₂O. The mixture was extracted with EtOAc, and the organic layer was dried over Na₂SO₄, filtered and concentrated in vacuo to give a residue which was purified by flash chromatography (0-10% EtOAc in petroleum ether) to afford title compound as a pale-yellow powder (167 mg, 53%). LCMS (ES⁺) Method 2: m/z 263 (M+H)⁺, RT 2.19 min.

### Step 2: methyl 4-bromo-2-(bromomethyl)-3-chlorobenzoate

A solution of methyl 4-bromo-3-chloro-2-methylbenzoate (167.0 mg, 0.63 mmol) in CHCl₃ (3.5 mL) was treated with NBS (169.2 mg, 0.95 mmol) and (E)-2,2′-(diazene-1,2-diyl)bis(2-methylpropanenitrile) (15.6 mg, 0.10 mmol) and the mixture was heated to reflux for 12 h. After cooling the reaction was quenched with H₂O and extracted in EtOAc. The organic layer was dried over Na₂SO₄, filtered, and concentrated in vacuo to give a residue which was purified by flash chromatography (0-15% EtOAc in petroleum ether) to afford title compound as a white powder (122 mg, 56%). LCMS (ES⁺) Method 2: m/z 341 (M+H)⁺, RT 2.20 min.

### Step 3: 5-bromo-4-chloro-2-methylisoindolin-1-one

A suspension of methyl 4-bromo-2-(bromomethyl)-3-chlorobenzoate (122.0 mg, 0.36 mmol) in THF (1 mL) was treated with methylamine (33% in EtOH; 1.0 mL, 6.41 mmol) and the suspension was heated at 50° C. for 16 h. After cooling, the mixture was diluted with HCl (1M aq. sol.) and extracted with EtOAc. The organic layer was dried over Na₂SO₄, filtered, and concentrated in vacuo to afford title compound as a white powder (70 mg, 75%). LCMS (ES⁺) Method 2: m/z 260, 262 (M+H)⁺, RT 1.51 min.

### Step 4: 4-chloro-2-methyl-5-(4,4,5,5-tetramethyl-1,3,2-dioxaborolan-2-yl)isoindolin-1-one

The title compound was prepared as reported in the synthesis of Intermediate 155 step 8. LCMS (ES⁺) Method 2: m/z 308 (M+H)⁺, RT 1.80 min.

### Intermediate 173: 5-fluoro-2-(2-((tetrahydro-2H-pyran-2-yl)oxy)ethyl)-6-(4,4,5,5-tetramethyl-1,3,2-dioxaborolan-2-yl)isoquinolin-1(2H)-one

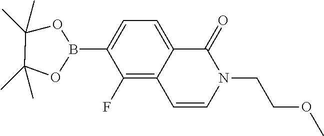

### Step 1: 6-bromo-5-fluoro-2-(2-((tetrahydro-2H-pyran-2-yl)oxy)ethyl)isoquinolin-1(2H)-one

A solution of 6-bromo-5-fluoroisoquinolin-1(2H)-one (100.0 mg, 0.41 mmol) in DMF (0.94 mL) was treated with K₂CO₃ (85.65 mg, 0.62 mmol), 2-(2-bromoethoxy)tetrahydro-2H-pyran (103.7 mg, 0.50 mmol) and KI (68.58 mg, 0.41 mmol) and heated at 80° C. for 48 h. After cooling the mixture was poured into H₂O and extracted with EtOAc. The organic layer was dried over Na₂SO₄, filtered, and concentrated in vacuo to give a residue which was purified by flash chromatography (0-100% EtOAc in petroleum ether) to afford title compound as a yellow oil (35 mg, 23%). LCMS (ES⁺) Method 2: m/z 370 (M+H)⁺, RT 2.01 min.

### Step 2: 5-fluoro-2-(2-((tetrahydro-2H-pyran-2-yl)oxy)ethyl)-6-(4,4,5,5-tetramethyl-1,3,2-dioxaborolan-2-yl)isoquinolin-1(2H)-one

The title compound was prepared as reported in the synthesis of Intermediate 155 step 8. LCMS (ES⁺) Method 2: m/z 440 (M+Na)⁺, RT 2.15 min.

### Intermediate 174: 5-fluoro-2-(2-methoxyethyl)-6-(4,4,5,5-tetramethyl-1,3,2-dioxaborolan-2-yl)isoquinolin-1(2H)-one

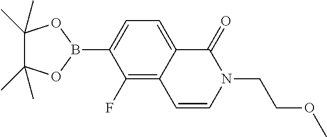

### Step 1: 6-bromo-5-fluoro-2-(2-methoxyethyl)isoquinolin-1(2H)-one

A solution of 6-bromo-5-fluoro-2-(2-hydroxyethyl)isoquinolin-1(2H)-one (prepared following the same procedure described for the synthesis of Intermediate 173 step 1; 54.0 mg, 0.19 mmol) in DMF (0.9 mL) was treated with NaH (60% in mineral oil; 9.81 mg, 0.25 mmol) and stirred for 10 min at 0° C. Mel (0.02 mL, 0.28 mmol) was added and the mixture stirred at rt for 2 h before being poured into H₂O and extracted with EtOAc. The organic layer was dried over Na₂SO₄, filtered, and concentrated in vacuo to afford title compound as a yellow oil (56 mg, 99%). LCMS (ES⁺) Method 2: m/z 300, 302 (M+H)⁺, RT 1.73 min.

### Step 2: 5-fluoro-2-(2-methoxyethyl)-6-(4,4,5,5-tetramethyl-1,3,2-dioxaborolan-2-yl)isoquinolin-1(2H)-one

The title compound was prepared as reported in the synthesis of Intermediate 155 step 8. LCMS (ES⁺) Method 2: m/z 348 (M+H)⁺, RT 1.91 min.

### Intermediate 175: 7-chloro-1,2-dimethyl-6-(4,4,5,5-tetramethyl-1,3,2-dioxaborolan-2-yl)-1H-benzo[d]imidazole

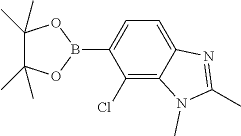

### Step 1: 5-bromo-6-chloro-N1-methylbenzene-1,2-diamine

A solution of 3-bromo-2-chloro-N-methyl-6-nitroaniline (422.0 mg, 0.79 mmol) in a mixture of THF (4 mL)/NH₄Cl (sat. sol.; 2.0 mL)/H₂O (2.0 mL) was treated with Zinc (519.6 mg, 7.95 mmol) and stirred for 1 h at rt. The reaction mixture was then filtered on a pad of cellulose and rinsed with EtOAc. H₂O was added to the filtrate, the phases were separated, and the aqueous phase was further extracted with EtOAc. The combined organics were washed with brine and dried over Na₂SO₄, filtered, and concentrated in vacuo to afford the title compound (58 mg, 31%). LCMS (ES⁺) Method 2: m/z 235, 237 (M+H)⁺, RT 1.60 min.

### Step 2: 6-bromo-7-chloro-1,2-dimethyl-1H-benzo[d]imidazole

A suspension of 5-bromo-6-chloro-N1-methylbenzene-1,2-diamine (58.0 mg, 0.25 mmol) in AcOH (0.2 mL) was heated at reflux for 12 h. After cooling to rt, the mixture was slowly basified to pH 9-10 with NH₄OH (28% aqueous solution) and extracted with EtOAc. The organic layer was dried over Na₂SO₄, filtered, and concentrated in vacuo to give a residue which was purified by flash chromatography (from 10-100% EtOAc in petroleum ether to 0-20% MeOH in EtOAc) to afford title compound as a brown powder (46 mg, 72%). LCMS (ES⁺) Method 2: m/z 259, 261 (M+H)⁺, RT 1.20 min.

### Step 3: 7-chloro-1,2-dimethyl-6-(4,4,5,5-tetramethyl-1,3,2-dioxaborolan-2-yl)-1H-benzo[d]imidazole

The title compound was prepared as reported in the synthesis of Intermediate 155 in step 8. LCMS (ES⁺) Method 2: m/z 307 (M+H)⁺, RT 1.33 min.

### Intermediate 176: 2,6-difluoro-4-(4,4,5,5-tetramethyl-1,3,2-dioxaborolan-2-yl)phenyl acetate

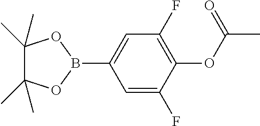 [SMILES: CC(=O)Oc1c(F)cc(B2OC(C)(C)C(C)(C)O2)cc1F]

### Step 1: 4-bromo-2,6-difluorophenyl acetate

A solution of 4-bromo-2,6-difluorophenol (0.5 g, 2.39 mmol) in DCM (48.0 mL) was treated with Ac₂O (0.57 mL, 5.98 mmol) and DMAP (584.6 mg, 4.78 mmol) and stirred at rt for 18 h before being diluted with H₂O and extracted with DCM. The organic layer was dried over Na₂SO₄, filtered, and concentrated in vacuo to give a residue which was purified by flash chromatography (0-30% EtOAc in petroleum ether) to afford title compound as a colorless oil (280 mg, 80% pure; 37%) and treated as reported in the synthesis of Intermediate 155 step 8. ¹H NMR (400 MHz, DMSO-d₆) δ 7.42-7.39 (br m, 2H), 2.42 (s, 3H), 1.31 (s, 6H), 1.17 (s, 6H).

### Intermediate 177a & 177b: 7-chloro-2-methyl-6-(4,4,5,5-tetramethyl-1,3,2-dioxaborolan-2-yl)-2H-indazole & 7-chloro-1-methyl-6-(4,4,5,5-tetramethyl-1,3,2-dioxaborolan-2-yl)-1H-indazole

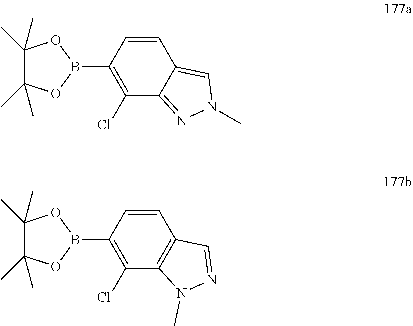

### Step 1: 6-bromo-7-chloro-2H-indazole

A solution of 4-bromo-3-chloro-2-fluorobenzaldehyde (200 mg, 0.84 mmol) in tBuOH (4.0 mL) was treated with N₂H₄·H₂O (157.0 uL, 5.05 mmol) and stirred at 100° C. for 48 h. After cooling the mixture was concentrated under reduced pressure to afford the title compound which was used without further purification (194 mg, 99%). LCMS (ES⁺) Method 2: m/z 231, 233 (M+H)⁺, RT 1.60 min.

### Step 2: 7-chloro-2-methyl-6-(4,4,5,5-tetramethyl-1,3,2-dioxaborolan-2-yl)-2H-indazole & 7-chloro-1-methyl-6-(4,4,5,5-tetramethyl-1,3,2-dioxaborolan-2-yl)-1H-indazole

The title compound was prepared following the procedure reported for the synthesis of Intermediate 171 in step 2 (mixture of regioisomers were obtained and separated by flash chromatography; 0-10% EtOAc in petroleum ether). The boronic esters derivatives were prepared as reported in the synthesis of Intermediate 155 step 8 to obtain Intermediates 177a & 177b. LCMS (ES⁺) Method 2: m/z 293 (M+H)⁺, RT 1.84 min, 2.22 min.

### Intermediate 178: 4-chloro-2-(oxetan-3-yl)-5-(4,4,5,5-tetramethyl-1,3,2-dioxaborolan-2-yl)-2H-indazole

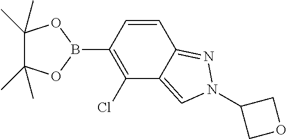 [SMILES: CC1(C)OB(c2ccc3nn(C4COC4)cc3c2Cl)OC1(C)C]

### Step 1: 5-bromo-4-chloro-2-(oxetan-3-yl)-2H-indazole

A solution of 3-bromooxetane (64.6 uL, 0.78 mmol) and 5-bromo-4-chloro-2H-indazole (150 mg, 0.65 mmol) in DMF (1.5 mL) was treated with Cs₂CO₃ (633.4 mg, 1.94 mmol) and stirred at 100° C. for 16 h. After cooling the mixture was diluted with H₂O and extracted with EtOAc. The organic layer was dried over Na₂SO₄, filtered, and concentrated in vacuo to give a residue which was purified by flash chromatography (0-20% EtOAc in petroleum ether) to afford title compound (50 mg, 27%). LCMS (ES⁺) Method 2: m/z 287, 289 (M+H)⁺, RT 1.83 min.

### Step 2: 4-chloro-2-(oxetan-3-yl)-5-(4,4,5,5-tetramethyl-1,3,2-dioxaborolan-2-yl)-2H-indazole

The title compound was prepared as reported in the synthesis of Intermediate 155 step 8. LCMS (ES⁺) Method 2: m/z 335 (M+H)⁺, RT 2.01 min.

### Intermediates 179a & 179b: 4-chloro-2-isopropyl-5-(4,4,5,5-tetramethyl-1,3,2-dioxaborolan-2-yl)-2H-indazole & 4-chloro-1-isopropyl-5-(4,4,5,5-tetramethyl-1,3,2-dioxaborolan-2-yl)-1H-indazole

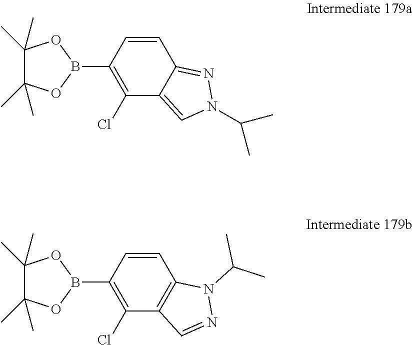

### Step 1: 5-bromo-4-chloro-2-isopropyl-2H-indazole & 5-bromo-4-chloro-1-isopropyl-1H-indazole

A solution of 5-bromo-4-chloro-2H-indazole (150 mg, 0.65 mmol) in DMF (0.4 mL) was treated with tBuOK (1M in THF; 0.97 mL, 0.97 mmol) and 2-iodopropane (0.1 mL, 0.97 mmol) and stirred at 80° C. for 24 h. Brine was added, and the mixture was extracted with EtOAc. The organic layer was dried over Na₂SO₄, filtered, and concentrated in vacuo to give a residue which was purified by flash chromatography (0-50% EtOAc in petroleum ether) to afford 5-bromo-4-chloro-2-isopropyl-2H-indazole (50 mg, 28%) and 5-bromo-4-chloro-1-isopropyl-1H-indazole (71 mg, 40%). LCMS (ES⁺) Method 2: m/z 273 (M+H)⁺, RT 2.17 min and 2.34 min.

### Step 2: 4-chloro-2-isopropyl-5-(4,4,5,5-tetramethyl-1,3,2-dioxaborolan-2-yl)-2H-indazole & 4-chloro-1-isopropyl-5-(4,4,5,5-tetramethyl-1,3,2-dioxaborolan-2-yl)-1H-indazole

The title compounds were prepared as reported in the synthesis of Intermediate 155 in step 8: Intermediate 179a; LCMS (ES⁺) Method 2: m/z 321 (M+H)⁺, RT 2.14 min and Intermediate 179b: LCMS (ES⁺) Method 2: m/z 321 (M+H)⁺, RT 2.48 min

### Intermediates 180a & 180b: 4-chloro-2-cyclopropyl-5-(4,4,5,5-tetramethyl-1,3,2-dioxaborolan-2-yl)-2H-indazole & 4-chloro-1-cyclopropyl-5-(4,4,5,5-tetramethyl-1,3,2-dioxaborolan-2-yl)-1H-indazole

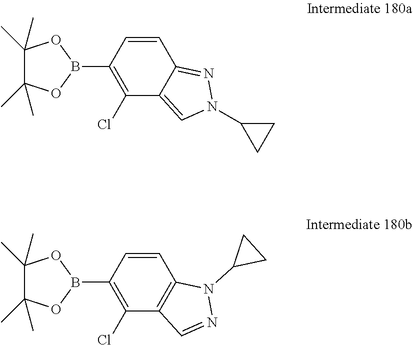 [SMILES: CC1(C)OB(c2ccc3nn(C4CC4)cc3c2Cl)OC1(C)C ; CC1(C)OB(c2ccc3c(cnn3C3CC3)c2Cl)OC1(C)C]

### Step 1: 5-bromo-4-chloro-2-cyclopropyl-2H-indazole & 5-bromo-4-chloro-1-cyclopropyl-1H-indazole

The title compounds were obtained using the procedure reported for the synthesis of Intermediate 178 step 1 (using bromocyclopropane in DMSO at 120° C.). LCMS (ES⁺) Method 2: m/z 271, 273 (M+H)⁺, RT 2.11 min and 2.26 min.

### Step 2: 4-chloro-2-cyclopropyl-5-(4,4,5,5-tetramethyl-1,3,2-dioxaborolan-2-yl)-2H-indazole & 4-chloro-1-cyclopropyl-5-(4,4,5,5-tetramethyl-1,3,2-dioxaborolan-2-yl)-1H-indazole

The title compounds were prepared as reported in the synthesis of Intermediate 155 step 8: Intermediate 180a; LCMS (ES⁺) Method 2: m/z 319 (M+H)⁺, RT 2.39 min and Intermediate 180b: LCMS (ES⁺) Method 2: m/z 319 (M+H)⁺, RT 2.19 min

### Intermediates 181a & 181b: 2-(4-chloro-5-(4,4,5,5-tetramethyl-1,3,2-dioxaborolan-2-yl)-2H-indazol-2-yl)-N,N-dimethylacetamide & 2-(4-chloro-5-(4,4,5,5-tetramethyl-1,3,2-dioxaborolan-2-yl)-1H-indazol-1-yl)-N,N-dimethylacetamide

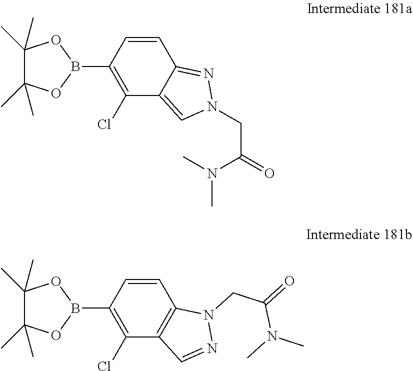

The title compounds were prepared as reported in the synthesis of Intermediate 178 using 2-chloro-N,N-dimethyl-acetamide in step 1. Intermediate 181a: LCMS (ES⁺) Method 2: m/z 364 (M+H)⁺, RT 1.81 min and Intermediate 181b: LCMS (ES⁺) Method 2: m/z 364 (M+H)⁺, RT 1.89 min

### Intermediate 182: 4-cyclopropyl-2-methyl-5-(4,4,5,5-tetramethyl-1,3,2-dioxaborolan-2-yl)-2H-indazole

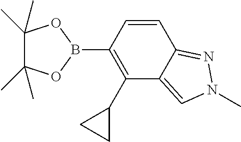

### Step 1: 4-bromo-3-cyclopropyl-2-methylaniline

A solution of 3-cyclopropyl-2-methyl-aniline (0.69 g, 3.42 mmol) in DMF (5 mL) at 0° C. was treated dropwise with a solution of NBS (609 mg, 3.42 mmol) in DMF (2 mL) and stirred for 30 min. The mixture was diluted with EtOAc, washed with Na₂CO₃ (10% aq. sol.) and brine. The organic layer was dried over Na₂SO₄, filtered, and concentrated in vacuo to give a residue which was purified by flash chromatography (0-20% EtOAc in petroleum ether) to afford title compound as an orange oil (720 mg, 73% pure, 68%). LCMS (ES⁺) Method 2: m/z 226, 228 (M+H)⁺, RT 1.73 min.

### Step 2: 5-bromo-4-cyclopropyl-2H-indazole

A solution of NaNO₂ (274.6 mg, 3.98 mmol) in H₂O (0.5 mL) was added dropwise to an ice bath cooled solution of 4-bromo-3-cyclopropyl-2-methylaniline (0.72 g, 3.18 mmol) in AcOH (13 mL). The resulting slurry was stirred for 1 h at rt. The solvent was partially removed under reduced pressure and the residue was diluted with EtOAc and washed with NaHCO₃ (sat. aq. sol.; 3×) and brine. The organic layer was dried over Na₂SO₄, filtered, and concentrated in vacuo to give a residue which was purified by flash chromatography (0-40% EtOAc in petroleum ether) to afford title compound as a yellow powder (540 mg, 71% pure, 51%). LCMS (ES⁺) Method 2: m/z 237 (M+H)⁺, RT 1.75 min.

### Step 3: 5-bromo-4-cyclopropyl-2-methyl-2H-indazole

A mixture of 5-bromo-4-cyclopropyl-2H-indazole (0.54 g, 2.28 mmol) in EtOAc (6 mL) was treated with trimethyloxonium tetrafluoroborate (0.37 g, 2.51 mmol) and stirred at rt for 4 h before being quenched with sat. NaHCO₃ (sat. aq. sol.) and extracted with EtOAc. The organic layer was washed with brine and dried over Na₂SO₄, filtered, and concentrated in vacuo to give a residue which was purified by flash chromatography (0-50% EtOAc in petroleum ether) to afford title compound as a brown oil (410 mg, 72%). LCMS (ES⁺) Method 2: m/z 251 (M+H)⁺, RT 1.75 min.

### Step 4: 4-cyclopropyl-2-methyl-5-(4,4,5,5-tetramethyl-1,3,2-dioxaborolan-2-yl)-2H-indazole

The title compound was prepared as reported in the synthesis of Intermediate 155 in step 8. LCMS (ES⁺) Method 2: m/z 299 (M+H)⁺, RT 1.81 min.

### Intermediate 183: (3-chloro-2-(cyclopropylamino)pyridin-4-yl)boronic acid

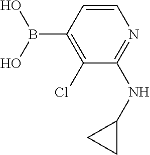 [SMILES: OB(O)c1ccnc(NC2CC2)c1Cl]

### Step 1: (3-chloro-2-(cyclopropylamino)pyridin-4-yl)boronic acid

The title compound was prepared as reported in the synthesis of Intermediate 155 in step 8 using 3-chloro-N-cyclopropyl-4-iodopyridin-2-amine. LCMS (ES⁺) Method 2: m/z 213 (M+H)⁺, RT 0.31 min.

### SYNTHESIS OF EXAMPLES

### Example 1: [3-[7-(aminomethyl)-7-(thiophen-3-yl)-3-azabicyclo[4.1.0]heptan-3-yl]-6-(2,3-dichlorophenyl)-5-methylpyrazin-2-yl]methanol (Trifluoro Acetate Salt)

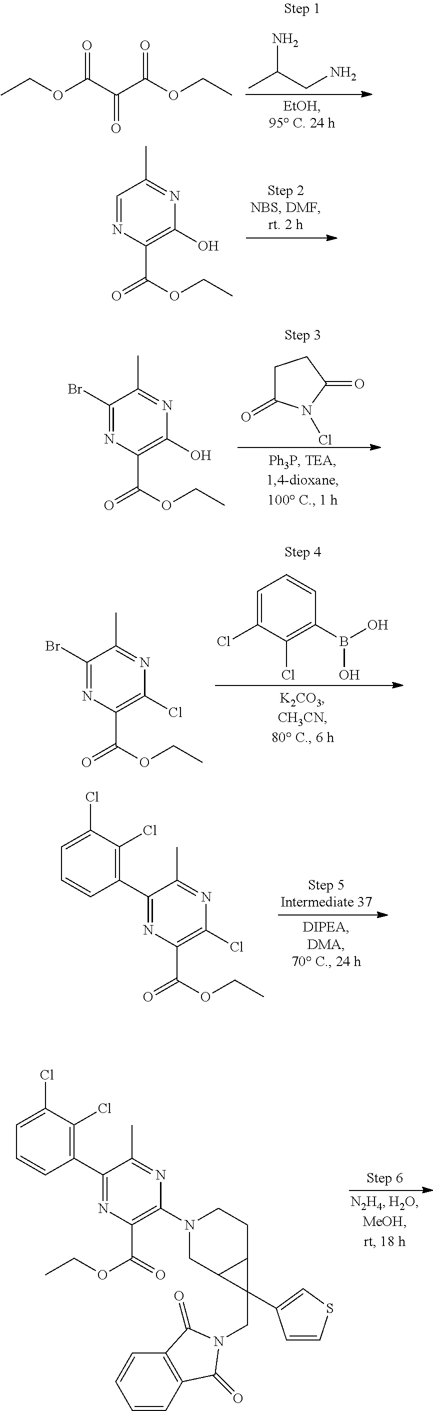

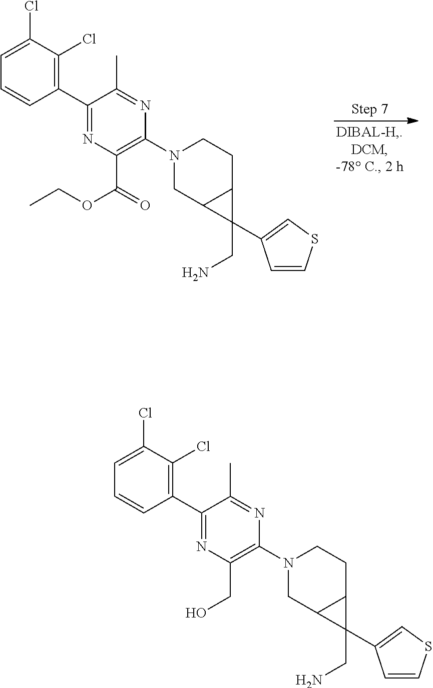 [SMILES: CCOC(=O)c1nc(-c2cccc(Cl)c2Cl)c(C)nc1N1CCC2C(C1)C2(CN)c1ccsc1 ; Cc1nc(N2CCC3C(C2)C3(CN)c2ccsc2)c(CO)nc1-c1cccc(Cl)c1Cl]

### Step 1: ethyl 3-hydroxy-5-methylpyrazine-2-carboxylate

A 50 ml flask was charged with EtOH (19.0 mL) and 1,2-diaminopropane (0.99 mL, 11.48 mmol) and the resulting clear, colourless solution was cooled to 0° C. Diethyl 2-oxopropanedioate (1.75 mL, 11.48 mmol) was added to the solution and the reaction mixture was allowed to warm to room temperature and stirred at this temperature for 2 h and at 95° C. for 24 h. After cooling to rt the reaction mixture was concentrated under reduced pressure to give a residue which was purified by flash chromatography (40-100% EtOAc in petroleum ether) to afford the title compound as a yellow solid (360 mg, 17%). ¹H NMR (400 MHz, DMSO-d₆) δ 12.83 (br s, 1H), 7.33 (br s, 1H), 4.26 (q, J=8.0 Hz, 2H), 2.24 (s, 3H), 1.27 (t, J=8.0 Hz, 3H). LCMS (ES⁺) Method 2: m/z 183 (M+H)⁺, RT 0.70 min.

### Step 2: ethyl 6-bromo-3-hydroxy-5-methylpyrazine-2-carboxylate

A 25 ml flask was charged with ethyl 3-hydroxy-5-methylpyrazine-2-carboxylate (300 mg, 1.65 mmol) in DMF (6.6 mL) and the resulting solution was cooled to 0° C. 1-Bromopyrrolidine-2,5-dione (307.7 mg, 1.73 mmol) was added and the reaction mixture warmed to rt and stirred for 2 h. The mixture was then diluted with H₂O and EtOAc. The organic phase was washed with brine, dried over Na₂SO₄, and concentrated in vacuo to afford the title compound as a crude (430 mg, 100%). LCMS (ES⁺) Method 2: m/z 261, 263 (M+H)⁺, RT 1.22 min.

### Step 3: ethyl 6-bromo-3-chloro-5-methylpyrazine-2-carboxylate

A 50 ml flask was charged with triphenylphosphine (1.3 g, 4.94 mmol) in 1,4-dioxane (15.0 mL) 1-chloropyrrolidine-2,5-dione (0.67 g, 5.02 mmol) was added and the resulting solution was stirred at rt for 30 min. Ethyl 6-bromo-3-hydroxy-5-methylpyrazine-2-carboxylate (430 mg, 1.65 mmol) was added and the resulting mixture was heated at 100° C. for 1 h. After cooling the mixture, TEA (4 mL, 1.65 mmol) was added. The volatiles were concentrated in vacuo and the residue was purified by flash chromatography (35% EtOAc in petroleum ether) to afford the title compound as a yellow oil (310 mg, 67%). ¹H NMR (400 MHz, CDCl₃) δ 4.49 (q, J=8.0 Hz, 2H), 2.74 (s, 3H), 1.44 (t, J=8.0 Hz, 3H). LCMS (ES⁺) Method 2: m/z 279, 281 (M+H)⁺, RT 1.81 min.

### Step 4: ethyl 3-chloro-6-(2,3-dichlorophenyl)-5-methylpyrazine-2-carboxylate

A degassed mixture of ethyl 6-bromo-3-chloro-5-methylpyrazine-2-carboxylate (500 mg, 1.79 mmol), (2,3-dichlorophenyl)boronic acid (410 mg, 2.15 mmol) and K₂CO₃ (980 mg, 7.09 mmol) in MeCN (17.9 mL) was treated with Pd(dppf)Cl₂·DCM (290 mg, 0.390 mmol). The reaction mixture was stirred at 80° C. for 6 h. After cooling, the reaction mixture was filtered on a pad of solka flok, the filtrate was concentrated in vacuo and purified by flash chromatography (0-30% EtOAc in petroleum ether) to afford the title compound (510 mg, 82%). ¹H NMR (400 MHz, CDCl₃) δ 7.60 (d, J=8.0 Hz, 1H), 7.36 (t, J=8.0 Hz, 1H), 7.28 (d, J=8.0 Hz, 1H), 4.49 (q, J=8.0 Hz, 2H), 2.48 (s, 3H), 1.44 (t, J=8.0 Hz, 3H). LCMS (ES⁺) Method 2: m/z 345-347 (M+H)⁺, RT 2.26 min.

### Step 5: ethyl 6-(2,3-dichlorophenyl)-3-(7-((1,3-dioxoisoindolin-2-yl)methyl)-7-(thiophen-3-yl)-3-azabicyclo[4.1.0]heptan-3-yl)-5-methylpyrazine-2-carboxylate

A mixture of ethyl 3-chloro-6-(2,3-dichlorophenyl)-5-methylpyrazine-2-carboxylate (18 mg, 0.050 mmol), Intermediate 37 (19.5 mg, 0.050 mmol) and DIPEA (0.04 mL, 0.210 mmol) in DMA (0.46 mL) was stirred at 70° C. for 24 h. After cooling, the volatiles were concentrated in vacuo and the residue (33 mg, 98%) was used without further purification. LCMS (ES⁺) Method 2: m/z 647, 649 (M+H)⁺, RT 2.69 min.

### Step 6: ethyl 3-(7-(aminomethyl)-7-(thiophen-3-yl)-3-azabicyclo[4.1.0]heptan-3-yl)-6-(2,3-dichlorophenyl)-5-methylpyrazine-2-carboxylate

Ethyl 6-(2,3-dichlorophenyl)-3-(7-((1,3-dioxoisoindolin-2-yl)methyl)-7-(thiophen-3-yl)-3-azabicyclo[4.1.0]heptan-3-yl)-5-methylpyrazine-2-carboxylate (33 mg, 0.050 mmol) in MeOH (0.48 mL) was treated with N₂H₄·H₂O (15.7 uL, 0.200 mmol). The resulting mixture was stirred at rt for 18 h then concentrated in vacuo to afford the title compound as a yellow powder (26 mg, 59%) and used without further purification. LCMS (ES⁺) Method 2: m/z 517, 519 (M+H)⁺, RT 1.59 min

### Step 7: (3-(7-(aminomethyl)-7-(thiophen-3-yl)-3-azabicyclo[4.1.0]heptan-3-yl)-6-(2,3-dichlorophenyl)-5-methylpyrazin-2-yl)methanol

A solution of ethyl 3-(7-(aminomethyl)-7-(thiophen-3-yl)-3-azabicyclo[4.1.0]heptan-3-yl)-6-(2,3-dichlorophenyl)-5-methylpyrazine-2-carboxylate (26 mg, 0.030 mmol) in DCM (0.30 mL) was treated with DIBAL-H (1M in THF; 0.12 mL, 0.120 mmol) at −78° C. The mixture was stirred at this temperature for 30 min then additional DIBAL-H (1M in THF; 0.12 mL, 0.120 mmol) was added and the mixture was stirred for further 1 h and 30 min at −78° C. DCM (0.6 mL) was added to the mixture followed by H₂O (40 uL) and 2N NaOH (10 uL). The resulting slurry was stirred 30 min then the mixture was filtered and washed with (DCM: MeOH/1: 5 mL). The filtrate was concentrated in vacuo and the residue was purified by preparative HPLC to obtain the title compound as a yellow solid (TFA salt; 0.9 mg, 6%). ¹H NMR (400 MHz, DMSO-d₆) δ 7.68 (d, J=8.0 Hz, 2H), 7.49-7.47 (m, 1H), 7.42 (t, J=8.0 Hz, 1H), 7.35-7.31 (m, 3H), 7.08 (d, J=8.0 Hz, 1H), 6.45 (s, 1H), 5.31 (br s, 1H), 4.50-4.44 (m, 2H), 3.83 (d, J=12.0 Hz, 1H), 3.69 (d, J=8.0 Hz, 1H), 3.52-3.50 (m, 2H), 2.65-2.59 (m, 1H), 2.21-2.12 (m, 2H), 2.15 (s, 3H), 1.95-1.89 (m, 1H), 1.75-1.68 (m, 1H), 1.53-1.49 (m, 1H). LCMS (ES⁺) Method 2: m/z 475 (M+H)⁺, RT 1.54 min

### Example 2: [3-[3-(2,3-dichlorophenyl)-1H-pyrazolo[3,4-b]pyrazin-6-yl]-7-(pyridin-3-yl)-3-azabicyclo[4.1.0]heptan-7-yl]methanamine (Trifluoro Acetate Salt)

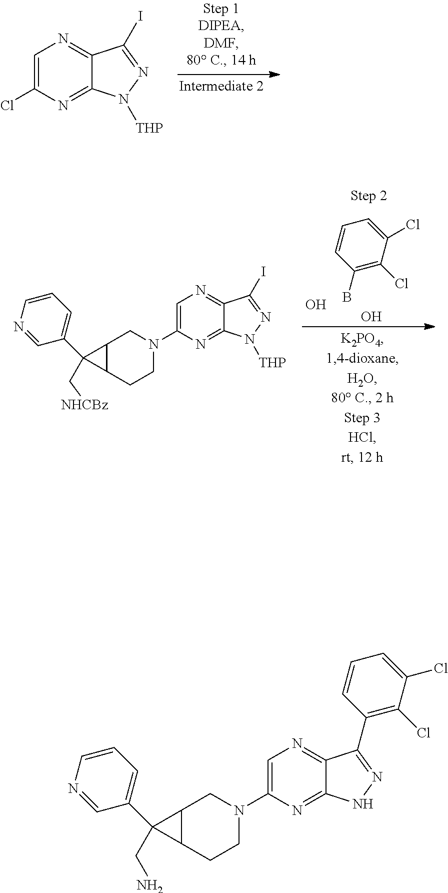

### Step 1: benzyl ((3-(3-iodo-1-(tetrahydro-2H-pyran-2-yl)-1H-pyrazolo[3,4-b]pyrazin-6-yl)-7-(pyridin-3-yl)-3-azabicyclo[4.1.0]heptan-7-yl)methyl)carbamate

A solution of Intermediate 44 (9 mg, 0.02 mmol) and Intermediate 2 (10 mg, 0.020 mmol) and DIPEA (26.4 uL, 0.150 mmol) in DMF (0.6 mL) was heated at 80° C. for 14 h. After cooling, EtOAc (20 mL) was added and the mixture washed with citric acid (5%), H₂O, brine, dried over Na₂SO₄ and concentrated in vacuo to afford the title compound as a yellow solid (14 mg, 85%) which was used as crude in the next step. LCMS (ES⁺) Method 2: m/z 666 (M+H)⁺, RT 1.59 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(pyridin-3-yl)-3-azabicyclo[4.1.0]heptan-7-yl)methyl)carbamate

A suspension of benzyl ((3-(3-iodo-1-(tetrahydro-2H-pyran-2-yl)-1H-pyrazolo[3,4-b]pyrazin-6-yl)-7-(pyridin-3-yl)-3-azabicyclo[4.1.0]heptan-7-yl)methyl)carbamate (15 mg, 0.020 mmol), (2,3-dichlorophenyl)boronic acid (8.6 mg, 0.050 mmol), Pd(dppf)Cl₂·DCM (3.73 mg, 0.005 mmol) and tripotassium phosphate (28.71 mg, 0.140 mmol) in a mixture of degassed 1,4-dioxane (0.7 mL) and H₂O (70 uL) was heated in a pressure tube at 80° C. for 2 h. After cooling, the mixture was diluted with EtOAc (20 mL), washed with brine (2×2 mL), dried over Na₂SO₄ and concentrated in vacuo to afford a residue which was purified by flash chromatography (0-100% EtOAc in petroleum ether) to obtain the title compound as a yellow solid (9 mg, 58%). LCMS (ES⁺) Method 2: m/z 684 (M+H)⁺, RT 1.94 min.

### Step 3: (3-(3-(2,3-dichlorophenyl)-1H-pyrazolo[3,4-b]pyrazin-6-yl)-7-(pyridin-3-yl)-3-azabicyclo[4.1.0]heptan-7-yl)methanamine

Benzyl ((3-(3-(2,3-dichlorophenyl)-1-(tetrahydro-2H-pyran-2-yl)-1H-pyrazolo[3,4-b]pyrazin-6-yl)-7-(pyridin-3-yl)-3-azabicyclo[4.1.0]heptan-7-yl)methyl)carbamate (9 mg, 0.010 mmol) was treated with conc. HCl (37%; 0.5 mL, 0.010 mmol) and the mixture was stirred at rt for 12 h. After volatiles removal in vacuo the residue was purified by preparative HPLC to obtain the title compound as a white solid (TFA salt; 0.4 mg, 7%). LCMS (ES⁺) Method 2: m/z 466 (M+H)⁺, RT 0.99 min.

The following examples were synthesized using the above procedure with the corresponding starting materials: Example 3, Example 4 and Example 5 (using Intermediate 3); Example 6 (using Intermediate 4); Example 7 (using Intermediate 5); Example 8, Example 9 (using Intermediate 6); Example 10, Example 11 and Example 12 (using Intermediate 7; for Example 12 the step 3 was performed using HCl (37%) in 1,4-dioxane); Example 13 (using Intermediate 9); Example 14 and Example 15 (using Intermediate 10 and 4N HCl in 1,4-dioxane in step 3 instead of 37% HCl, and for Example 15 the Intermediate 45 was used in step 2); Example 16 (using Intermediate 3 and Intermediate 11); Example 17 (using Intermediate 6 and Intermediate 11); Example 18, Example 19, Example 20, Example 21, Example 22, Example 23, Example 24 (using Intermediate 12 and for Example 24 the Intermediate 45 was used in step 2); Example 25 Example 26, Example 27, Example 28, Example 29, Example 30, Example 31 (using Intermediate 6); Example 32 (using Intermediate 6 and HBr (33% in AcOH) was used in step 3 instead of 37% HCl); Example 33 (using Intermediate 6 and Intermediate 15); Example 34 (using Intermediate 6 and Intermediate 16); Example 35 (using Intermediate 6 and HBr (33% in AcOH) was used in step 3 instead of 37% HCl); Example 36 (using Intermediate 6 and Intermediate 17); Example 37, Example 38, Example 39, Example 40 (using Intermediate 18); Example 41 (using Intermediate 18 and Intermediate 19); Example 42 (using Intermediate 18 and Intermediate 20); Example 43 (using Intermediate 18 and Intermediate 21); Example 44 (using Intermediate 18 and Intermediate 46); Example 45 (using Intermediate 18 and Intermediate 22); Example 46 (using Intermediate 18 and Intermediate 45); Example 47 (using Intermediate 23 and Intermediate 45); Example 48 (using Intermediate 24 and Intermediate 45); Example 49 (using Intermediate 25 and Intermediate 45); Example 50 (using Intermediate 26 and Intermediate 45); Example 51 (using Intermediate 18 and Intermediate 27); Example 52 (using Intermediate 10).

| Example # | ¹H NMR (400 MHz, DMSO-d₆) δ |
|---|---|
| 3 | δ 13.47 (br s, 1H), 8.43 (s, 1H), 7.78-7.69 (m, 5H), 7.53-7.49 (m, 2H), 7.40-7.35 (m, 1H), |
| | 7.23-7.19 (m, 2H), 4.44 (d, J = 12.0 Hz, 1H), 4.35-4.32 (m, 1H), 3.18-3.07 (m, 2H), 2.97- |
| | 2.89 (m, 1H), 2.36-2.33 (m, 1H), 1.82-1.76 (m, 2H), 1.57-1.54 (m, 1H), 1.19-1.16 (m, 1H) |
| 4 | 13.51 (br s, 1H), 8.89 (d, J = 4.0 Hz, 1H), 8.53 (s, 1H), 7.96 (d, J = 4.0 Hz, 1H), 7.85 (d, |
| | J = 4.0 Hz, 1H), 7.66-7.32 (m, 7H), 7.22 (d, J = 4.0 Hz, 2H), 4.47 (d, J = 16.0 Hz, 1H), 4.39- |
| | 4.37 (m, 1H), 3.79-3.75 (m, 1H), 3.14-2.95 (m, 4H), 1.82-1.79 (m, 2H), 1.59-1.56 (m, 1H) |
| 5 | 13.55 (br s, 1H), 9.30 (d, J = 8.0 Hz, 1H), 9.01 (s, 1H), 8.49 (s, 1H), 8.42 (d, J = 8.0 Hz, 1H), |
| | 8.13 (d, J = 8.0 Hz, 1H), 7.96 (t, J = 8.0 Hz, 1H), 7.67-7.64 (m, 4H), 7.48-7.45 (m, 1H), |
| | 7.37-7.35 (m, 1H), 7.23-7.19 (m, 2H), 4.47 (d, J = 16.0 Hz, 1H), 4.37-4.34 (m, 1H), 3.79- |
| | 3.73 (m, 1H), 3.18-2.95 (m, 4H), 1.84-1.80 (m, 2H), 1.57-1.54 (m, 1H) |
| 6 | 13.47 (br s, 1H), 8.40 (s, 1H), 7.93 (br s, 3H), 7.89 (d, J = 8.0 Hz, 1H), 7.68 (d, J = 8.0 Hz, |
| | 1H), 7.50 (t, J = 8.0 Hz, 1H), 6.28 (s, 1H), 4.33 (d, J = 12.0 Hz, 2H), 3.71-3.69 (m, 1H), |
| | 3.26-3.22 (m, 2H), 2.97-2.94 (m, 1H), 2.32-2.30 (m, 1H), 2.21 (s, 3H), 2.08-1.71 (m, 3H) |
| 7 | 8.33 (s, 1H), 7.69 (d, J = 8.0 Hz, 1H), 7.62 (d, J = 8.0 Hz, 1H), 7.54 (br s, 3H), 7.43 (t, J = 8.0 |
| | Hz, 1H), 7.40-7.32 (m, 1H), 7.25 (t, J = 8.0 Hz, 2H), 7.06 (t, J = 8.0 Hz, 1H), 4.35 (d, J = 16.0 |
| | Hz, 1H), 4.26 (dd, J = 4.0, 12 Hz, 1H), 3.64-3.61 (m, 1H), 3.22-3.18 (m, 1H), 3.10-3.06 |
| | (m, 1H), 2.84-2.81 (m, 1H), 2.27-2.22 (m, 1H), 1.80-1.48 (m, 3H) |
| 8 | 13.47 (br s, 1H), 8.40 (s, 1H), 7.87 (br s, 3H), 7.76 (d, J = 8.0 Hz, 1H), 7.69 (d, J = 8.0 Hz, |
| | 1H), 7.51 (t, J = 8.0 Hz, 1H), 6.15 (s, 1H), 4.33-4.29 (m, 2H), 3.75-3.70 (m, 1H), 3.27-3.18 |
| | (m, 2H), 2.99-2.92 (m, 1H), 2.39 (s, 3H), 2.31-2.28 (m, 1H), 1.98-1.92 (m, 1H), 1.78-1.72 |
| | (m, 2H) |
| 9 | 13.56 (br s, 1H), 9.28 (d, J = 8.0 Hz, 1H), 9.00 (s, 1H), 8.46 (s, 1H), 8.42 (d, J = 8.0 Hz, |
| | 1H), 8.13 (d, J = 8.0 Hz, 1H), 7.95 (t, J = 8.0 Hz, 1H), 7.88 (br s, 3H), 7.66-7.63 (m, 1H), |
| | 6.15 (s, 1H), 4.34 (d, J = 16.0 Hz, 2H), 3.77-3.72 (m, 2H), 3.25-3.20 (m, 1H), 2.99-2.95 |
| | (m, 1H), 2.39 (s, 3H), 2.31-2.28 (m, 1H), 1.99-1.95 (m, 1H), 1.77-1.73 (m, 2H) |
| 10 | 8.39 (s, 1H), 7.74 (d, J = 8.0 Hz, 1H), 7.65 (d, J = 8.0 Hz, 1H), 7.60 (br s, 3H), 7.53-7.47 |
| | (m, 3H), 7.18 (t, J = 8.0 Hz, 2H), 4.42 (d, J = 12.0 Hz, 1H), 4.31 (br, J = 16 Hz, 1H), 3.72- |
| | 3.67 (m, 1H), 3.25-3.20 (m, 1H), 3.15-3.10 (m, 1H), 2.92-2.85 (m, 1H), 1.80-1.75 (m, |
| | 2H), 1.52-1.50 (m, 1H) |
| 11 | 13.51 (s, 1H), 9.17 (s, 1H), 8.97 (d, J = 16.0 Hz, 2H), 8.76 (d, J = 8.0 Hz, 1H), 8.56 (s, 1H), |
| | 8.23 (d, J = 8.0 Hz, 1H), 7.59 (br s, 3H), 7.52 (t, J = 8.0 Hz, 2H), 7.21 (t, J = 8.0 Hz, 2H), |
| | 4.45 (d, J = 16.0 Hz, 1H), 4.35 (br d, J = 16 Hz, 1H), 3.79-3.76 (m, 1H), 3.27-3.21 (m, 2H), |
| | 3.15-3.10 (m, 1H), 2.96-2.90 (m, 1H), 1.84-1.75 (m, 2H), 1.57-1.51 (m, 1H) |
| 12 | 9.04 (s, 1H), 8.67 (d, J = 8.0 Hz, 1H), 8.65 (br s, 1H), 8.45 (s, 1H), 8.18 (d, J = 8.0 Hz, 1H), |
| | 7.86 (t, J = 8.0 Hz, 1H), 7.76 (br s, 1H), 7.64 (br s, 3H), 7.53 (t, J = 8.0 Hz, 2H), 7.21 (t, |
| | J = 8.0 Hz, 2H), 4.48 (d, J = 16.0 Hz, 1H), 4.36 (br d, J = 12 Hz, 1H), 3.28-3.24 (m, 2H), |
| | 3.17-3.15 (m, 1H), 2.97-2.88 (m, 1H), 2.35-2.31 (m, 1H), 1.84-1.75 (m, 2H), 1.54-1.51 |
| | (m, 1H) |
| 13 | 8.40 (s, 1H),7.93 (s, 1H), 7.83 (br s, 3H), 7.76 (d, J = 8.0 Hz, 1H), 7.69 (d, J = 8.0 Hz, 1H), |
| | 7.51(t, J = 8.0 Hz, 1H), 4.31 (br d, J = 12.0 Hz, 2H), 4.03 (s, 3H), 3.77-3.72 (m, 2H), 3.25- |
| | 3.20 (m, 1H), 2.98-2.81 (m, 1H), 2.33-2.30 (m, 1H), 1.89-1.62 (m, 3H) |
| 14 | 13.47 (br s, 1H), 8.67 (s, 1H), 8.42 (s, 1H), 7.82 (br s, 3H), 7.75 (d, J = 8.0 Hz, 1H), 7.66 |
| | (d, J = 8.0 Hz, 1H), 7.50 (t, J = 8.0 Hz, 1H), 4.41 (d, J = 12.0 Hz, 1H), 4.34 (d, J = 12.0 Hz, |
| | 1H), 3.31-3.14 (m, 2H), 2.95-2.92 (m, 1H), 2.35-2.32 (m, 1H), 2.08 (s, 3H), 2.01-1.98 (m, |
| | 1H), 1.76-1.71 (m, 2H) |
| 15 | 13.36 (br s, 1H), 8.68 (s, 1H), 8.56 (s, 1H), 8.42 (s, 1H), 7.79 (br s, 3H), 7.69-7.63 (m, |
| | 2H), 4.43 (d, J = 12.0 Hz, 1H), 4.34 (d, J = 12.0 Hz, 1H), 4.23 (s, 3H), 3.76-3.72 (m 1H), |
| | 3.24-3.12 (m, 2H), 2.95-2.92 (m, 1H), 2.35-2.32 (m, 1H), 2.08 (s, 3H), 2.01-1.98 (m, 1H), |
| | 1.76-1.71 (m, 2H) |
| 16 | 13.54 (br s, 1H), 8.45 (s, 1H), 8.08 (d, J = 4.0 Hz, 1H), 7.67 (br s, 3H), 7.47-7.21 (m, 4H), |
| | 7.02 (d, J = 4.0 Hz, 1H), 6.85 (br s, 1H), 4.44 (d, J = 16.0 Hz, 1H), 4.33 (d, J = 12.0 Hz, 1H), |
| | 3.76-3.72 (m 1H), 3.18-3.10 (m, 2H), 2.95-2.93 (m, 1H), 2.93 (s, 3H), 2.35-2.32 (m, 1H), |
| | 1.92-1.77 (m, 2H), 1.57-1.51 (m, 1H) |
| 17 | 8.48 (s, 1H), 8.08 (d, J = 8.0 Hz, 1H), 7.88 (br s, 3H), 7.55 (d, J = 8.0 Hz, 1H), 6.12 (s, 1H), |
| | 4.31-4.26 (m, 2H), 3.79-3.75 (m, 1H), 3.40-3.37 (m, 1H), 3.24-3.19 (m, 1H), 3.06 (s, 3H), |
| | 3.02-2.97 (m, 1H), 2.35-2.32 (m, 2H), 1.97-1.94 (m, 2H), 1.78-1.71 (m, 3H) |
| 18 | 3.89 (s, 1H), 8.87 (d, J = 4.0 Hz, 1H), 8.71 (d, J = 4.0 Hz, 1H), 8.56 (s, 1H), 8.24 (d, J = 4.0 |
| | Hz, 1H), 7.86 (br s, 3H), 7.65 (d, J = 4.0 Hz, 1H), 6.15 (s, 1H), 4.35 (d, J = 12.0 Hz, 2H), |
| | 3.82-3.78 (m, 1H), 3.40-3.20 (m, 2H), 3.04-3.02 (m, 1H), 2.39 (s, 3H), 2.35-2.32 (m, 1H), |
| | 1.97-1.94 (m, 1H), 1.78-1.71 (m, 2H) |
| 19 | 13.49 (s, 1H), 8.91 (d, J = 8.0 Hz, 1H), 8.69 (s, 1H), 8.49 (s, 1H), 7.86 (br s, 3H), 7.72-7.66 |
| | (m, 2H), 6.15 (s, 1H), 4.73 (s, 2H), 4.33 (d, J = 16.0 Hz, 2H), 3.79-3.75 (m, 1H), 3.30-3.20 |
| | (m, 2H), 3.04-2.95 (m, 1H), 2.39 (s, 3H), 2.35-2.32 (m, 1H), 1.97-1.94 (m, 1H), 1.78-1.71 |
| | (m, 2H) |
| 20 | 13.34 (s, 1H), 9.45 (s, 1H), 9.12 (s, 1H), 8.50 (s, 1H), 8.39 (d, J = 8.0 Hz, 1H), 8.28 (d, |
| | J = 8.0 Hz, 1H), 7.87 (br s, 3H), 6.15 (s, 1H), 4.35 (d, J = 12.0 Hz, 2H), 3.78-3.73 (m, 1H), |
| | 3.25-3.21 (m, 2H), 3.04-2.95 (m, 1H), 2.39 (s, 3H), 2.35-2.32 (m, 1H), 1.98-1.95 (m, 1H), |
| | 1.78-1.72 (m, 2H) |
| 21 | 13.48 (s, 1H), 9.23 (d, J = 8.0 Hz, 1H), 9.00 (d, J = 4.0 Hz, 1H), 8.45 (s, 1H), 8.26 (d, J = 8.0 |
| | Hz, 1H), 7.88 (br s, 3H), 7.79 (d, J = 8.0 Hz, 1H), 7.63-7.59 (m, 1H), 6.16 (s, 1H), 4.35 (d, |
| | J = 16.0 Hz, 2H), 3.79-3.73 (m, 1H), 3.45-3.41 (m, 1H), 3.25-3.21 (m, 1H), 3.04-2.95 (m, |
| | 1H), 2.81 (s, 3H), 2.40 (s, 3H), 2.35-2.32 (m, 1H), 2.00-1.94 (m, 1H), 1.78-1.72 (m, 2H) |
| 22 | 13.42 (br s, 1H), 9.33 (br d, J = 8.0 Hz, 1H), 8.94 (s, 1H), 8.44 (s, 1H), 8.37 (d, J = 8.0 Hz, |
| | 1H), 7.88 (br s, 2H), 7.68 (d, J = 8.0 Hz, 1H), 7.42 (d, J = 8.0 Hz, 1H), 6.16 (s, 1H), 4.35 |
| | (d, J = 12.0 Hz, 2H), 4.07 (s, 3H), 3.78-3.73 (m, 1H), 3.27-3.21 (m, 2H), 3.02-2.96 (m, |
| | 1H), 2.40 (s, 3H), 2.35-2.32 (m, 1H), 1.98-1.94 (m, 1H), 1.79-1.75 (m, 2H) |
| 23 | 13.60 (br s, 1H), 9.53 (s, 1H), 8.87 (br s, 1H), 8.71 (br d, J = 4.0 Hz, 1H), 8.61 (d, J = 8.0 |
| | Hz, 1H), 8.48 (s, 1H), 8.30 (d, J = 8.0 Hz, 1H), 7.94 (d, J = 8.0 Hz, 1H), 7.88 (br s, 3H), |
| | 6.16 (s, 1H), 4.35 (d, J = 12.0 Hz, 2H), 3.80-3.75 (m, 1H), 3.30-3.21 (m, 2H), 3.03-2.97 |
| | (m, 1H), 2.40 (s, 3H), 2.35-2.32 (m, 1H), 2.00-1.94 (m, 1H), 1.79-1.75 (m, 2H) |
| 24 | 13.40 (br s, 1H), 8.56 (s, 1H), 8.40 (s, 1H), 7.89 (br s, 3H), 7.70-7.63 (m, 2H), 6.15 (s, |
| | 1H), 4.32 (d, J = 16.0 Hz, 2H), 4.24 (s, 3H), 3.75-3.70 (m, 1H), 3.42-3.40 (m, 1H), 3.26- |
| | 3.21 (m, 1H), 3.03-2.92 (m, 1H), 2.39 (s, 3H), 2.35-2.32 (m, 1H), 1.98-1.94 (m, 1H), 1.79- |
| | 1.72 (m, 2H) |
| 26 | 13.37 (s, 1H), 8.81 (s, 1H), 8.47 (s, 1H), 8.46 (d, J = 8.0 Hz, 1H), 7.90 (br s, 3H), 7.63 (d, |
| | J = 8.0 Hz, 1H), 7.38 (t, J = 8.0 Hz, 1H), 6.15 (s, 1H), 4.34 (d, J = 16.0 Hz, 2H), 4.24 (s, 3H), |
| | 3.75-3.70 (m, 1H), 3.42-3.40 (m, 1H), 3.26-3.21 (m, 1H), 3.03-2.92 (m, 1H), 2.39 (s, 3H), |
| | 2.35-2.32 (m, 1H), 1.98-1.94 (m, 1H), 1.79-1.72 (m, 2H) |
| 27 | 13.84 (s, 1H), 9.11-9.07 (m, 2H), 8.52 (s, 1H), 8.43 (br d, J = 4.0 Hz, 1H), 8.15 (d, J = 8.0 |
| | Hz, 1H), 7.90-7.87 (br m, 4H), 7.73 (t, J = 8.0 Hz, 1H), 6.16 (s, 1H), 4.35 (d, J = 12.0 Hz, |
| | 2H), 3.80-3.75 (m, 1H), 3.45-3.40 (m, 1H), 3.25-3.21 (m, 1H), 3.04-2.98 (m, 1H), 2.40 (s, |
| | 3H), 2.35-2.32 (m, 1H), 2.00-1.96 (m, 1H), 1.79-1.74 (m, 2H) |
| 28 | 8.40 (s, 1H), 8.25 (br s, 2H), 7.87 (br s, 3H), 7.14 (br t, J = 8.0 Hz, 1H), 6.14 (s, 1H), 4.32 |
| | (d, J = 16.0 Hz, 2H), 3.90 (s, 3H), 3.74-3.72 (m, 1H), 3.40-3.19 (m, 2H), 2.97-2.94 (m, |
| | 1H), 2.38 (s, 3H), 2.35-2.32 (m, 1H), 1.97-1.93 (m, 1H), 1.76-1.73 (m, 2H) |
| 29 | 8.45 (s, 1H), 7.87 (br s, 3H), 7.75 (d, J = 4.0 Hz, 1H), 7.37 (br s, 1H), 6.14 (s, 1H), 4.64 |
| | (br s, J = 8.0 Hz, 1H), 4.32 (d, J = 16.0 Hz, 2H), 3.81-3.72 (m, 3H), 3.56-3.52 (m, 2H), 3.40- |
| | 3.19 (m, 2H), 3.00-2.94 (m, 1H), 2.39 (s, 3H), 2.35-2.32 (m, 1H), 2.16-2.14 (m, 2H), 2.00- |
| | 1.89 (m, 2H), 1.80-1.73 (m, 2H), 1.58-1.52 (m, 1H) |
| 30 | 13.55 (s, 1H), 9.31 (d, J = 8.0 Hz, 1H), 9.04 (d, J = 4.0 Hz, 1H), 8.46 (s, 1H), 8.38 (br t, |
| | J = 8.0 Hz, 1H), 7.89 (br s, 3H), 7.77 (t, J = 8.0 Hz, 1H), 7.73-7.69 (m, 1H), 6.16 (s, 1H), |
| | 4.34 (d, J = 16.0 Hz, 2H), 3.79-3.73 (m, 1H), 3.40-3.19 (m, 2H), 3.03-2.96 (m, 1H), 2.40 |
| | (s, 3H), 2.35-2.32 (m, 1H), 1.99-1.95 (m, 1H), 1.79-1.75 (m, 2H) |
| 31 | 13.78 (s, 1H), 8.52 (d, J = 4.0 Hz, 1H), 8.47 (s, 1H), 8.00 (d, J = 4.0 Hz, 1H), 7.86 (br s, |
| | 3H), 6.14 (s, 1H), 4.31 (d, J = 16.0 Hz, 2H), 3.78-3.73 (m, 1H), 3.40-3.19 (m, 2H), 3.01- |
| | 2.95 (m, 1H), 2.39 (s, 3H), 2.35-2.32 (m, 1H), 1.97-1.94 (m, 1H), 1.77-1.72 (m, 2H) |
| 32 | 13.81 (s, 1H), 9.52 (d, J = 8.0 Hz, 1H), 9.16 (d, J = 4.0 Hz, 1H), 8.62 (d, J = 8.0 Hz, 1H), |
| | 8.55 (d, J = 8.0 Hz, 1H), 8.51 (s, 1H), 7.88 (br s, 3H), 7.83-7.79 (m, 1H), 6.16 (s, 1H), 4.35 |
| | (d, J = 12.0 Hz, 2H), 3.82-3.77 (m, 1H), 3.40-3.19 (m, 2H), 3.04-2.98 (m, 1H), 2.40 (s, |
| | 3H), 2.35-2.31 (m, 1H), 1.99-1.94 (m, 1H), 1.79-1.74 (m, 2H) |
| 33 | 13.60 (s, 1H), 9.38 (d, J = 4.0 Hz, 1H), 9.09 (d, J = 4.0 Hz, 1H), 8.47 (s, 1H), 8.38 (d, J = 4.0 |
| | Hz, 1H), 8.15 (d, J = 4.0 Hz, 1H), 7.87 (br s, 3H), 7.74-7.71 (m, 1H), 6.15 (s, 1H), 4.34 (d, |
| | J = 16.0 Hz, 2H), 3.79-3.73 (m, 1H), 3.40-3.20 (m, 2H), 3.04-2.98 (m, 1H), 2.39 (s, 3H), |
| | 2.35-2.31 (m, 1H), 1.99-1.95 (m, 1H), 1.79-1.74 (m, 2H) |
| 35 | 9.15 (d, J = 4.0 Hz, 1H), 9.00 (br s, 1H), 8.54 (d, J = 8.0 Hz, 1H), 8.40 (br s, 1H), 8.05 (br |
| | s, 1H), 7.88 (br m, 4H), 6.16 (s, 1H), 4.35 (br d, J = 16.0 Hz, 2H), 3.76-3.72 (m, 1H), 3.40- |
| | 3.20 (m, 2H), 3.02-2.96 (m, 1H), 2.40 (s, 3H), 2.35-2.31 (m, 1H), 1.98-1.95 (m, 1H), 1.79- |
| | 1.74 (m, 2H) |
| 36 | 8.49 (s, 1H), 8.09 (d, J = 8.0 Hz, 1H), 7.89 (br s, 3H), 7.65 (d, J = 8.0 Hz, 1H), 6.11 (s, 1H), |
| | 4.30 (d, J = 16.0 Hz, 2H), 3.79-3.74 (m, 1H), 3.40-3.35 (m, 1H), 3.25-3.19 (m, 1H), 3.01- |
| | 2.97 (m, 1H), 2.37 (s, 3H), 2.33-2.28 (m, 1H), 1.98-1.93 (m, 1H), 1.77-1.72 (m, 2H) |
| 37 | 13.35 (s, 1H), 8.45 (s, 1H), 8.33 (d, J = 8.0 Hz, 1H), 8.20 (s, 1H), 7.87 (br s, 3H), 7.56 (d, |
| | J = 8.0 Hz, 1H), 6.14 (s, 1H), 4.32 (d, J = 16.0 Hz, 2H), 3.79-3.73 (m, 1H), 3.43-3.38 (m, |
| | 1H), 3.20-3.15 (m, 1H), 3.00-2.99 (m, 1H), 2.38 (s, 3H), 2.33-2.30 (m, 1H), 1.98-1.94 (m, |
| | 1H), 1.76-1.72 (m, 2H) |
| 38 | 13.87 (s, 1H), 9.53 (d, J = 8.0 Hz, 1H), 9.25 (d, J = 8.0 Hz, 1H), 9.16 (s, 1H), 8.54 (s, 1H), |
| | 8.52 (d, J = 8.0 Hz, 1H), 7.87 (br s, 3H), 7.54 (d, J = 8.0 Hz, 1H), 6.16 (s, 1H), 4.35 (d, |
| | J = 12.0 Hz, 2H), 3.82-3.79 (m, 1H), 3.43-3.15 (m, 2H), 3.00-2.97 (m, 1H), 2.40 (s, 3H), |
| | 2.33-2.30 (m, 1H), 1.98-1.94 (m, 1H), 1.79-1.74 (m, 2H) |
| 39 | 13.83 (s, 1H), 9.07 (s, 1H), 8.93 (d, J = 8.0 Hz, 1H), 8.54 (s, 2H), 8.21-8.19 (m, 1H), 7.87 |
| | (br s, 3H), 7.78-7.75 (m, 1H), 6.16 (s, 1H), 4.36 (d, J = 16.0 Hz, 2H), 3.82-3.79 (m, 1H), |
| | 3.43-3.22 (m, 2H), 3.01-2.98 (m, 1H), 2.40 (s, 3H), 2.33-2.30 (m, 1H), 1.98-1.94 (m, 1H), |
| | 1.79-1.74 (m, 2H) |
| 40 | 8.38 (s, 1H), 7.87 (br s, 3H), 7.39 (br s, 1H), 6.93 (s, 2H), 6.14 (s, 1H), 4.34-4.25 (m, 6H), |
| | 3.72-3.67 (m, 1H), 3.45-3.40 (m, 1H), 3.29-3.25 (m, 1H), 2.95-2.92 (m, 1H), 2.38 (s, 3H), |
| | 2.31-2.28 (m, 1H), 1.98-1.94 (m, 1H), 1.76-1.71 (m, 2H) |
| 41 | 13.46 (s, 1H), 9.06 (s, 1H), 8.32 (s, 1H), 8.02 (d, J = 8.0 Hz, 1H), 7.88-7.80 (m, 4H), 7.64 |
| | (s, 1H), 7.40-7.35 (m, 1H), 6.14 (s, 1H), 4.35-4.26 (m, 2H), 3.82-3.79 (m, 1H), 3.43-3.22 |
| | (m, 2H), 2.95-2.94 (m, 1H), 2.40 (s, 3H), 2.33-2.30 (m, 1H), 1.98-1.94 (m, 1H), 1.76-1.72 |
| | (m, 2H) |
| 42 | 13.85 (s, 1H), 9.31 (s, 1H), 9.10 (d, J = 8.0 Hz, 1H), 8.58 (s, 1H), 8.54 (s, 1H), 8.15 (d, |
| | J = 8.0 Hz, 1H), 7.88-7.86 (m, 4H), 6.16 (s, 1H), 4.36 (d, J = 12.0 Hz, 2H), 3.82-3.79 (m, |
| | 1H), 3.43-3.22 (m, 2H), 3.02-2.99 (m, 1H), 2.40 (s, 3H), 2.33-2.30 (m, 1H), 1.98-1.94 (m, |
| | 1H), 1.78-1.74 (m, 2H) |
| 43 | 13.74 (s, 1H), 9.41 (d, J = 8.0 Hz, 1H), 9.12 (s, 1H), 8.53-8.50 (m, 2H), 8.37 (d, J = 8.0 Hz, |
| | 1H), 7.89 (br s, 3H), 7.78-7.75 (m, 1H), 6.16 (s, 1H), 4.36 (d, J = 12.0 Hz, 2H), 3.82-3.79 |
| | (m, 1H), 3.45-3.22 (m, 2H), 3.02-2.99 (m, 1H), 2.40 (s, 3H), 2.35-2.31 (m, 1H), 1.98-1.95 |
| | (m, 1H), 1.78-1.74 (m, 2H) |
| 44 | 13.43 (s, 1H), 8.39 (s, 1H), 7.89 (br s, 3H), 7.70 (d, J = 8 Hz, 1H), 7.61 (d, J = 8 Hz, 1H), |
| | 6.14 (s, 1H), 4.31 (d, J = 16.0 Hz, 2H), 4.18 (s, 3H), 3.76-3.70 (m, 1H), 3.45-3.22 (m, 2H), |
| | 2.98-2.96 (m, 1H), 2.38 (s, 3H), 2.30-2.26 (m, 1H), 1.97-1.94 (m, 1H), 1.76-1.72 (m, 2H) |
| 45 | 13.83 (s, 1H), 9.16 (t, J = 8 Hz, 1H), 9.09 (d, J = 4 Hz, 1H), 8.52 (s, 1H), 8.39 (d, J = 4 Hz, |
| | 1H), 7.88-7.84 (br m, 4H), 7.64 (t, J = 8 Hz, 1H), 6.15 (s, 1H), 4.35 (d, J = 12.0 Hz, 2H), |
| | 3.80-3.75 (m, 1H), 3.45-3.22 (m, 2H), 3.02-2.98 (m, 1H), 2.39 (s, 3H), 2.30-2.26 (m, 1H), |
| | 1.98-1.94 (m, 1H), 1.78-1.74 (m, 2H) |
| 46 | 8.52 (s, 1H), 8.38 (s, 1H), 7.87 (br s, 3H), 7.64 (t, J = 8 Hz, 2H), 6.11 (s, 1H), 4.33 (d, |
| | J = 12.0 Hz, 2H), 4.22 (s, 3H), 3.73-3.70 (m, 1H), 3.44-3.40 (m, 1H), 3.26-3.21 (m, 1H), |
| | 2.99-2.96 (m, 1H), 2.37 (s, 3H), 2.33-2.27 (m, 1H), 1.96-1.92 (m, 1H), 1.76-1.71 (m, 2H) |
| 47 | 13.37 (s, 1H), 8.56 (s, 1H), 8.40 (s, 1H), 7.89 (br s, 3H), 7.69-7.62 (m, 2H), 6.44 (s, 1H), |
| | 4.52 (s, 2H), 4.33 (d, J = 12.0 Hz, 2H), 4.23 (s, 3H), 3.77-3.73 (m, 1H), 3.45-3.42 (m, 1H), |
| | 3.33 (s, 3H), 3.31-3.22 (m, 1H), 2.98-2.95 (m, 1H), 2.35-2.32 (m, 1H), 2.01-1.95 (m, 1H), |
| | 1.81-1.75 (m, 2H) |
| 48 | 13.37 (s, 1H), 8.55 (s, 1H), 8.39 (s, 1H), 7.86 (br s, 3H), 7.69-7.62 (m, 2H), 6.11 (s, 1H), |
| | 4.32 (d, J = 12.0 Hz, 2H), 4.23 (s, 3H), 3.75-3.72 (m, 1H), 3.45-3.42 (m, 1H), 3.27-3.22 |
| | (m, 1H), 2.98-2.95 (m, 1H), 2.35-2.32 (m, 1H), 2.10-2.00 (m, 1H), 1.95-1.89 (m, 1H), |
| | 1.76-1.71 (m, 2H), 1.10-1.06 (m, 2H), 0.85-0.82 (m, 2H) |
| 49 | 8.54 (s, 1H), 8.43 (s, 1H), 8.41 (s, 1H), 7.82-7.75 (m, 4H), 7.68-7.64 (m, 2H), 7.49-7.46 |
| | (m, 1H), 4.49 (d, J = 12.0 Hz, 1H), 4.39 (d, J = 12.0 Hz, 1H), 4.23 (s, 3H), 3.76-3.71 (m, |
| | 1H), 3.34-3.21 (m, 2H), 2.95-2.93 (m, 1H), 2.35-2.31 (m, 1H), 2.10-2.06 (m, 1H), 1.85- |
| | 1.80 (m, 2H) |
| 50 | 13.37 (s, 1H), 8.56 (s, 1H), 8.50 (s, 1H), 8.41 (s, 1H), 7.79-7.77 (m, 4H), 7.68-7.64 (m, |
| | 2H), 7.34-7.31 (m, 1H), 4.37-4.34 (m, 2H), 4.24 (s, 3H), 3.79-3.72 (m, 1H), 3.56-3.49 (m, |
| | 2H), 3.01-2.95 (m, 1H), 2.38-2.32 (m, 1H), 2.08-2.03 (m, 1H), 1.85-1.76 (m, 2H) |
| 51 | 13.60 (s, 1H), 8.47 (s, 1H), 8.42 (d, J = 8 Hz, 1H), 7.91 (br s, 3H), 7.76 (t, J = 8 Hz, 1H), |
| | 7.25 (d, J = 8 Hz, 1H), 6.15 (s, 1H), 4.33 (d, J = 16 Hz, 2H), 3.78-3.74 (m, 1H), 3.56-3.30 |
| | (m, 2H), 3.22 (s, 3H), 3.01-2.95 (m, 1H), 2.38 (s, 3H), 2.35-2.32 (m, 1H), 1.99-1.94 (m, |
| | 1H), 1.77-1.72 (m, 2H) |
| 52 | 13.33 (s, 1H), 8.77 (s, 1H), 8.68 (s, 1H), 8.62 (d, J = 8 Hz, 1H), 8.53 (s, 1H), 7.86-7.80 (m, |
| | 4H), 4.44 (d, J = 16 Hz, 1H), 4.41-4.36 (m, 1H), 3.81-3.78 (m, 1H), 3.30-3.19 (m, 2H), |
| | 2.98-2.95 (m, 1H), 2.64 (s, 3H), 2.37-2.32 (m, 1H), 2.09 (s, 3H), 2.05-2.02 (m, 1H), 1.76- |
| | 1.72 (m, 2H) |

### Example 53: 4-((6-(7-(aminomethyl)-7-(5-methylisoxazol-3-yl)-3-azabicyclo[4.1.0]heptan-3-yl)-1H-pyrazolo[3,4-b]pyrazin-3-yl)thio)-3-chloropyridin-2-amine (Trifluoro Acetate Salt)

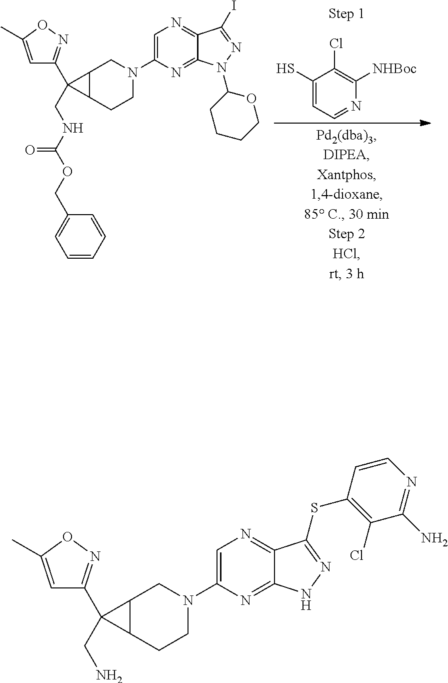 [SMILES: Cc1cc(C2(CNC(=O)OCc3ccccc3)C3CCN(c4cnc5c(I)nn(C6CCCCO6)c5n4)CC32)no1 ; CC(C)(C)OC(=O)Nc1nccc(S)c1Cl ; Cc1cc(C2(CN)C3CCN(c4cnc5c(Sc6ccnc(N)c6Cl)n[nH]c5n4)CC32)no1]

### Step 1: benzyl ((3-(3-((2-((tert-butoxycarbonyl)amino)-3-chloropyridin-4-yl)thio)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate

A suspension of benzyl ((3-(3-iodo-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (prepared with the procedure reported for Example 2 step 1; 28.4 mg, 0.040 mmol, using Intermediate 6 (12.2 mg, 0.050 mmol), Pd₂(dba)₃ (3.9 mg, 0.004 mmol), XantPhos (2.5 mg, 0.004 mmol) and DIPEA (11.0 mg, 0.085 mmol) in 1,4-dioxane (0.6 mL) was heated at 85° C. for 1 h. After cooling, the solvent was concentrated in vacuo and EtOAc and H₂O were added. The organic phase was washed with brine, dried over Na₂SO₄, and concentrated in vacuo to get the title compound as a brown oil (34 mg, 100%) which was used as crude in the next step. LCMS (ES⁺) Method 2: m/z 803 (M+H)⁺, RT 2.05 min.

### Step 2: 4-((6-(7-(aminomethyl)-7-(5-methylisoxazol-3-yl)-3-azabicyclo[4.1.0]heptan-3-yl)-1H-pyrazolo[3,4-b]pyrazin-3-yl)thio)-3-chloropyridin-2-amine

Benzyl ((3-(3-((2-((tert-butoxycarbonyl)amino)-3-chloropyridin-4-yl)thio)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (34 mg, 0.040 mmol) was treated with conc. HCl (37%; 0.5 mL, 0.040 mmol) and the mixture was stirred at rt for 3 h. After solvent removal in vacuo the residue was purified by preparative HPLC to obtain the title compound as a white solid (TFA salt; 1.0 mg, 5%). ¹H NMR (400 MHz, DMSO-d₆) δ 13.90 (s, 1H), 8.41 (s, 1H), 7.86 (br s, 3H), 7.58 (d, J=4.0 Hz, 1H), 6.51 (br s, 2H), 6.14 (s, 1H), 5.76 (s, 2H), 4.33-4.26 (m, 2H), 3.76-3.72 (m, 1H), 3.29-3.21 (m, 2H), 2.98-2.89 (m, 1H), 2.39 (s, 3H), 2.34-2.29 (m, 1H), 1.99-1.92 (m, 1H), 1.77-1.74 (m, 2H). LCMS (ES⁺) Method 2: m/z 484 (M+H)⁺, RT 0.78 min

### Example 54: (7-(5-methylisoxazol-3-yl)-3-(3-(thieno[3,2-b]pyridin-7-ylthio)-1H-pyrazolo[3,4-b]pyrazin-6-yl)-3-azabicyclo[4.1.0]heptan-7-yl)methanamine (Trifluoro Acetate Salt)

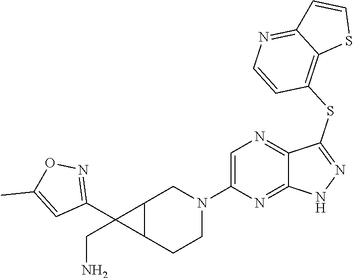 [SMILES: Cc1cc(C2(CN)C3CCN(c4cnc5c(Sc6ccnc7ccsc67)n[nH]c5n4)CC32)no1]

Example 54 was synthesized using Intermediate 14 (instead of tert-butyl (3-chloro-4-mercaptopyridin-2-yl)carbamate) and benzyl ((3-(3-iodo-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate with the same procedure reported for Example 53. ¹H NMR (400 MHz, DMSO-d₆) δ 7.79 (d, J=8.0 Hz, 1H), 7.72 (d, J=8.0 Hz, 1H), 7.65 (br s, 3H), 7.59-7.53 (m, 1H), 7.48-7.43 (m, 1H), 7.35-7.33 (m, 2H), 7.15-7.12 (m, 1H), 5.50-5.47 (m, 1H), 4.61 (d, J=8.0 Hz, 1H), 4.01-3.95 (m, 2H), 3.66-3.57 (m, 2H), 2.70-2.68 (m, 1H), 2.47-2.45 (m, 2H), 2.33-2.28 (m, 1H), 1.82-1.80 (m, 2H), 1.77-1.74 (m, 1H).

### Example 55: (6-(7-(aminomethyl)-7-(3-fluorophenyl)-3-azabicyclo[4.1.0]heptan-3-yl)-3-(2,3-dichlorophenyl)-1H-pyrazolo[3,4-b]pyrazin-5-yl)methanol (Trifluoro Acetate Salt) & Example 56: (6-(7-(aminomethyl)-7-(3-fluorophenyl)-3-azabicyclo[4.1.0]heptan-3-yl)-3-phenyl-1H-pyrazolo[3,4-b]pyrazin-5-yl)methanol (Trifluoro Acetate Salt)

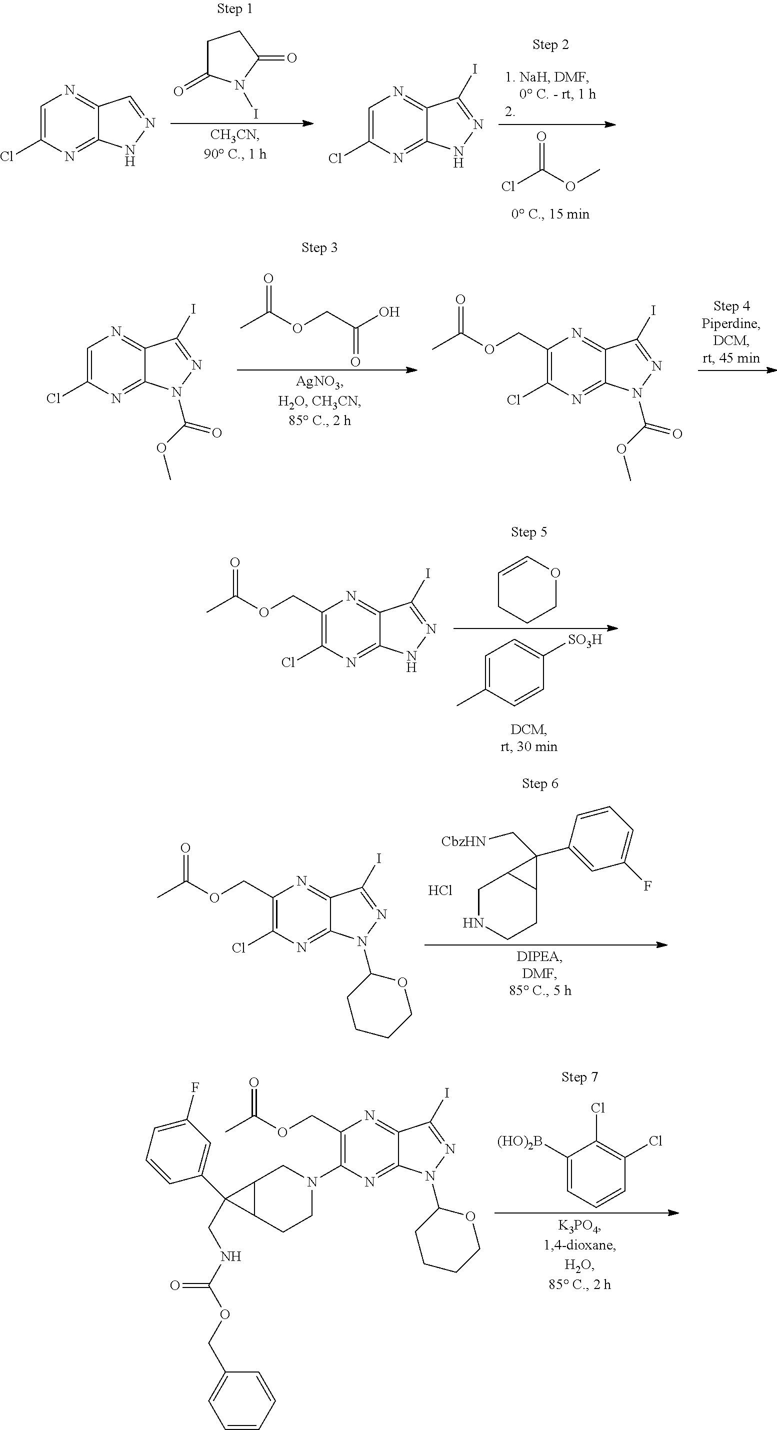

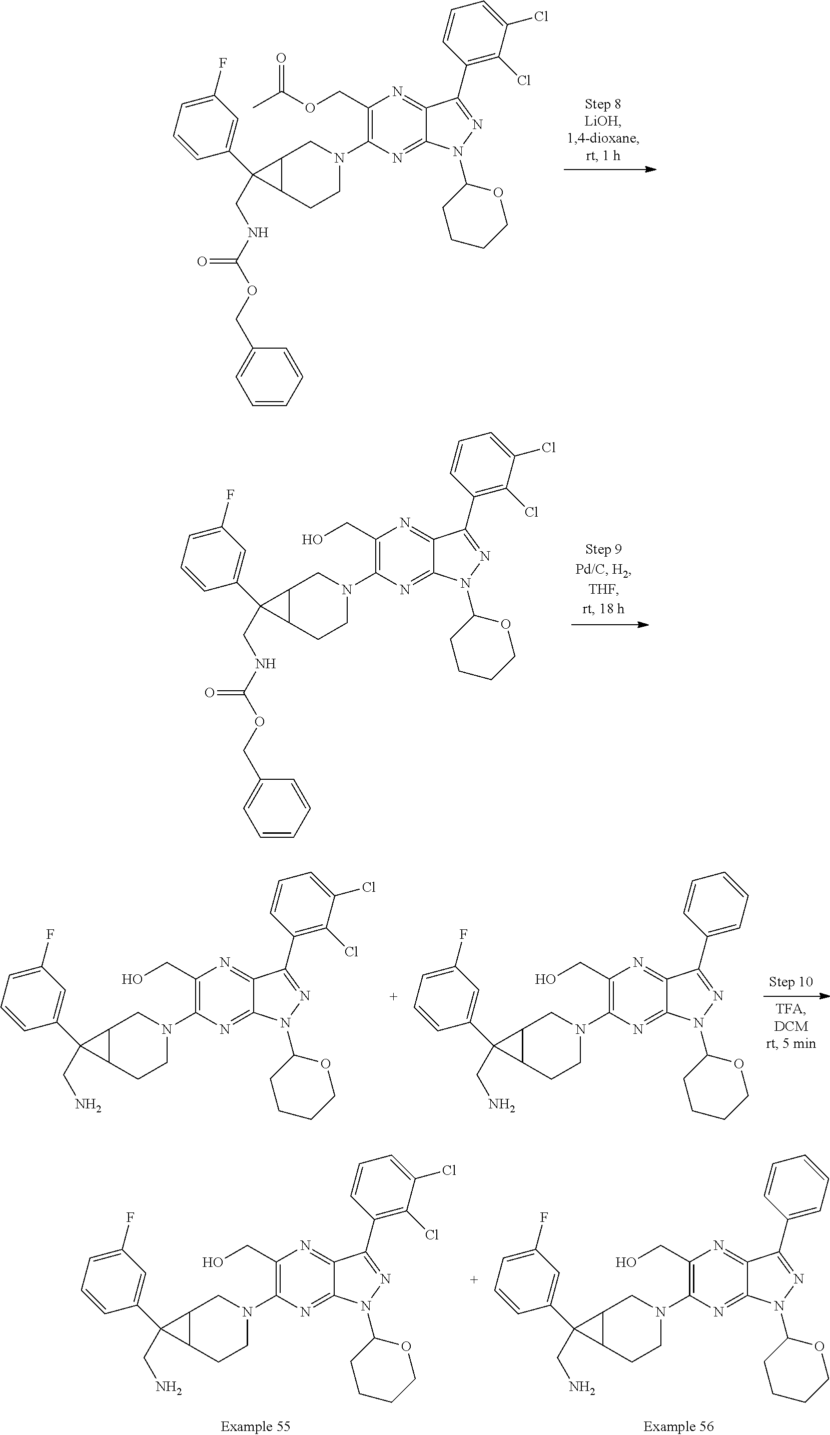

### Step 1: 6-chloro-3-iodo-1H-pyrazolo[3,4-b]pyrazine

A solution of 6-chloro-1H-pyrazolo[3,4-b]pyrazine (1.5 g, 9.71 mmol) in MeCN (32.0 mL) was treated with 1-iodopyrrolidine-2,5-dione (4.37 g, 19.41 mmol) and trifluoroborane hydrofluoride (3.8 mL, 29.12 mmol) and the resulting brown solution was stirred at 90° C. for 1 h. After cooling to rt the mixture was placed in an ice bath for 30 min. The formed precipitate was collected by filtration and washed with cold MeCN to afford the title compound as a yellow solid (3.0 g, 97%). ¹H NMR (400 MHz, DMSO-d₆) δ 8.71 (s, 1H).

### Step 2: methyl 6-chloro-3-iodo-1H-pyrazolo[3,4-b]pyrazine-1-carboxylate

A solution of NaH (60% dispersion in mineral oil; 941 mg, 23.53 mmol) in DMF (21 mL) was cooled to 0° C. and treated with 6-chloro-3-iodo-1H-pyrazolo[3,4-b]pyrazine (3.0 g, 10.7 mmol). After stirring at rt for 1 h, the mixture was cooled to 0° C. and treated with methyl chloroformate (3.03 g, 32.09 mmol) and stirred at rt for 15 min. The solution was then poured in H₂O and the precipitate was collected by filtration to afford the title compound as a brown solid (2.49 g, 69%). ¹H NMR (400 MHz, DMSO-d₆) δ 8.93 (s, 1H), 4.07 (s, 3H). LCMS (ES⁺) Method 2: m/z 338 (M+H)⁺, RT 1.40 min

### Step 3: methyl 5-(acetoxymethyl)-6-chloro-3-iodo-1H-pyrazolo[3,4-b]pyrazine-1-carboxylate

A solution of methyl 6-chloro-3-iodo-1H-pyrazolo[3,4-b]pyrazine-1-carboxylate (500 mg, 1.48 mmol), 2-acetyloxyethanoic acid (1.07 mL, 11.82 mmol) and AgNO₃ (50.2 mg, 0.300 mmol) in MeCN (15 mL) and H₂O (9 mL) at 85° C. was treated with APS (2.7 g, 11.82 mmol) and heated at 85° C. for 2 h. After cooling to rt the mixture was poured into EtOAc and brine. The organic layer was separated and purified by flash chromatography (0-60% EtOAc in petroleum ether) to afford the title compound as a yellow solid (330 mg, 54%). LCMS (ES⁺) Method 2: m/z 411 (M+H)⁺, RT 1.50 min

### Step 4: (6-chloro-3-iodo-1H-pyrazolo[3,4-b]pyrazin-5-yl)methyl acetate

A solution of methyl 5-(acetoxymethyl)-6-chloro-3-iodo-1H-pyrazolo[3,4-b]pyrazine-1-carboxylate (310 mg, 0.760 mmol) in DCM (5.6 mL) was treated with piperidine (0.09 mL, 0.910 mmol) and stirred at rt for 45 min. The solvent was concentrated in vacuo and the residue purified by flash chromatography (0-50% EtOAc in petroleum ether) to afford the title compound as a yellow solid (130 mg, 49%). LCMS (ES⁺) Method 2: m/z 353 (M+H)⁺, RT 1.40 min

### Step 5: (6-chloro-3-iodo-1-(tetrahydro-2H-pyran-2-yl)-1H-pyrazolo[3,4-b]pyrazin-5-yl)methyl acetate

A solution of (6-chloro-3-iodo-1H-pyrazolo[3,4-b]pyrazin-5-yl)methyl acetate (130 mg, 0.370 mmol) in DCM (1.8 mL) was treated with DHP (0.1 mL, 1.11 mmol), 4-methylbenzenesulfonic acid (19.0 mg, 0.110 mmol) and stirred at rt for 30 min. The solvent was concentrated in vacuo and the residue was purified by flash chromatography (0-50% EtOAc in petroleum ether) to afford the title compound as a pale yellow solid (52 mg, 32%). ¹H NMR (400 MHz, DMSO-d₆) δ 5.92 (dd, J=4.2, 8.0 Hz, 1H), 5.42 (q, J=12.0 Hz, 2H), 3.95-3.93 (m, 1H), 3.76-3.70 (m, 1H), 2.45-2.40 (m, 1H), 2.15 (s, 3H), 2.05-1.94 (m, 2H), 1.80-1.74 (m, 1H), 1.61-1.44 (m, 2H). LCMS (ES⁺) Method 2: m/z 437 (M+H)⁺, RT 1.98 min

### Step 6: (6-(7-((((benzyloxy)carbonyl)amino)methyl)-7-(3-fluorophenyl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate

A solution of (6-chloro-3-iodo-1-(tetrahydro-2H-pyran-2-yl)-1H-pyrazolo[3,4-b]pyrazin-5-yl)methyl acetate (29 mg, 0.070 mmol), benzyl ((7-(3-fluorophenyl)-3-azabicyclo[4.1.0]heptan-7-yl)methyl)carbamate hydrochloride (25.9 mg, 0.070 mmol) and DIPEA (71 uL, 0.400 mmol) in DMF (1.33 mL) was stirred at 85° C. for 5 h. After cooling EtOAc (20 mL) was added and the mixture washed with 5% citric acid (3 mL), H₂O (3 mL), brine (3 mL), dried over Na₂SO₄, and concentrated in vacuo to afford the title compound as a brown oil (50 mg, 99%) which was used in the next step without further purification. LCMS (ES⁺) Method 2: m/z 755 (M+H)⁺, RT 2.51-2.53 min

### Step 7: (6-(7-((((benzyloxy)carbonyl)amino)methyl)-7-(3-fluorophenyl)-3-azabicyclo[4.1.0]heptan-3-yl)-3-(2,3-dichlorophenyl)-1-(tetrahydro-2H-pyran-2-yl)-1H-pyrazolo[3,4-b]pyrazin-5-yl)methyl acetate

A solution of (6-(7-((((benzyloxy)carbonyl)amino)methyl)-7-(3-fluorophenyl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate (50 mg, 0.070 mmol), (2,3-dichlorophenyl)boronic acid (25.3 mg, 0.130 mmol), Pd(dppf)Cl₂·DCM (10.9 mg, 0.010 mmol) and K₃PO₄ (84.4 mg, 0.400 mmol) in a degassed mixture of 1,4-dioxane/H₂O 10/1 (0.88 mL) was heated in a pressure tube at 85° C. for 2 h. After cooling, the mixture was diluted with EtOAc (25 mL), washed with brine (2×5 mL), dried over Na₂SO₄, and concentrated in vacuo to give a residue which was purified by flash chromatography (0-100% EtOAc in petroleum ether) to afford the title compound as a yellow solid (43 mg, 84%). LCMS (ES⁺) Method 2: m/z 773 (M+H)⁺, RT 2.33-2.35 min

### Step 8: benzyl ((3-(3-(2,3-dichlorophenyl)-5-(hydroxymethyl)-1-(tetrahydro-2H-pyran-2-yl)-1H-pyrazolo[3,4-b]pyrazin-6-yl)-7-(3-fluorophenyl)-3-azabicyclo[4.1.0]heptan-7-yl)methyl)carbamate

A solution of (6-(7-((((benzyloxy)carbonyl)amino)methyl)-7-(3-fluorophenyl)-3-azabicyclo[4.1.0]heptan-3-yl)-3-(2,3-dichlorophenyl)-1-(tetrahydro-2H-pyran-2-yl)-1H-pyrazolo[3,4-b]pyrazin-5-yl)methyl acetate (43 mg, 0.060 mmol) in 1,4-dioxane (0.32 mL) was treated with a solution of LiOH (9.3 mg, 0.220 mmol) in H₂O (0.32 mL). The mixture was stirred at rt for 1 h. EtOAc and brine were added to the mixture and then the organic phase was isolated, dried over Na₂SO₄, and concentrated in vacuo to afford the title compound as a yellow oil (40 mg, 98%) which was used without further purification. LCMS (ES⁺) Method 2: m/z 731 (M+H)⁺, RT 2.27-2.29 min

### Step 9: (6-(7-(aminomethyl)-7-(3-fluorophenyl)-3-azabicyclo[4.1.0]heptan-3-yl)-3-(2,3-dichlorophenyl)-1-(tetrahydro-2H-pyran-2-yl)-1H-pyrazolo[3,4-b]pyrazin-5-yl)methanol and (6-(7-(aminomethyl)-7-(3-fluorophenyl)-3-azabicyclo[4.1.0]heptan-3-yl)-3-phenyl-1-(tetrahydro-2H-pyran-2-yl)-1H-pyrazolo[3,4-b]pyrazin-5-yl)methanol

A solution of benzyl ((3-(3-(2,3-dichlorophenyl)-5-(hydroxymethyl)-1-(tetrahydro-2H-pyran-2-yl)-1H-pyrazolo[3,4-b]pyrazin-6-yl)-7-(3-fluorophenyl)-3-azabicyclo[4.1.0]heptan-7-yl)methyl)carbamate (40 mg, 0.050 mmol) in THF (0.5 mL) was treated with Pd/C (10 mg) and the mixture was stirred under H₂ atmosphere for 18 h. After fluxing N₂, the catalyst was filtered on a pad of solka-floc and washed with MeOH. After removal of solvent the title compounds were obtained in a mixture 1:1 and used in the next step without further purification. LCMS (ES⁺) Method 2: m/z 529 (M+H)⁺, RT 1.40-1.43 min, m/z 597 (M+H)⁺ RT 1.45-1.49 min

### Step 10: (6-(7-(aminomethyl)-7-(3-fluorophenyl)-3-azabicyclo[4.1.0]heptan-3-yl)-3-(2,3-dichlorophenyl)-1H-pyrazolo[3,4-b]pyrazin-5-yl)methanol and (6-(7-(aminomethyl)-7-(3-fluorophenyl)-3-azabicyclo[4.1.0]heptan-3-yl)-3-phenyl-1H-pyrazolo[3,4-b]pyrazin-5-yl)methanol

A solution of a mixture of (6-(7-(aminomethyl)-7-(3-fluorophenyl)-3-azabicyclo[4.1.0]heptan-3-yl)-3-(2,3-dichlorophenyl)-1-(tetrahydro-2H-pyran-2-yl)-1H-pyrazolo[3,4-b]pyrazin-5-yl)methanol and (6-(7-(aminomethyl)-7-(3-fluorophenyl)-3-azabicyclo[4.1.0]heptan-3-yl)-3-phenyl-1-(tetrahydro-2H-pyran-2-yl)-1H-pyrazolo[3,4-b]pyrazin-5-yl)methanol (34 mg, 0.06 mmol) in DCM (0.70 mL) was treated with TFA (0.27 mL) and the mixture was stirred at rt for 5 min. The solvent was concentrated in vacuo and the residue was purified by preparative HPLC (15-100% MeCN/H₂O+0.1% TFA) to obtain the title compounds as yellow solids. Example 55: (TFA salt; 0.7 mg, 2%); ¹H NMR (400 MHz, DMSO-d₆) δ 7.79 (d, J=8.0 Hz, 1H), 7.72 (d, J=8.0 Hz, 1H), 7.65 (br s, 3H), 7.59-7.53 (m, 1H), 7.48-7.43 (m, 1H), 7.35-7.33 (m, 2H), 7.15-7.12 (m, 1H), 5.50-5.47 (m, 1H), 4.61 (d, J=8.0 Hz, 1H), 4.01-3.95 (m, 2H), 3.66-3.57 (m, 2H), 2.70-2.68 (m, 1H), 2.47-2.45 (m, 2H), 2.33-2.28 (m, 1H), 1.82-1.80 (m, 2H), 1.77-1.74 (m, 1H). LCMS (ES⁺) Method 2: m/z 513 (M+H)⁺, RT 1.30 min. Example 56: (TFA salt; 0.88 mg, 3%); ¹H NMR (400 MHz, DMSO-d₆) δ 8.44 (d, J=8.0 Hz, 2H), 7.69-7.65 (br s, 3H), 7.53-7.51 (m, 2H), 7.44-7.42 (m, 2H), 7.32-7.30 (m, 2H), 7.15-7.12 (m, 1H), 5.56-5.54 (m, 1H), 4.70 (d, J=4.0 Hz, 2H), 4.60 (m, 1H), 4.01-3.89 (m, 2H), 3.64-3.60 (m, 2H), 2.70-2.67 (m, 1H), 2.33-2.30 (m, 1H), 2.10-1.99 (m, 1H), 1.82-1.80 (m, 1H), 1.65-1.63 (m, 1H). LCMS (ES⁺) Method 2: m/z 445 (M+H)⁺, RT 1.19 min

### Example 57: (3-((2-amino-3-chloropyridin-4-yl)thio)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol (Trifluoro Acetate Salt)

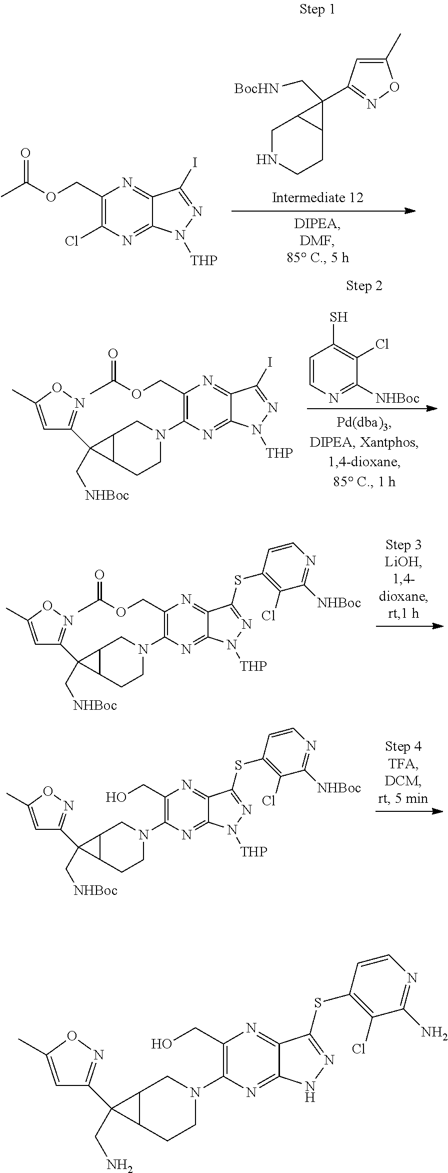

### Step 1: (6-(7-(((tert-butoxycarbonyl)amino)methyl)-7-(5-methylisoxazol-3-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate

A solution of (6-chloro-3-iodo-1-(tetrahydro-2H-pyran-2-yl)-1H-pyrazolo[3,4-b]pyrazin-5-yl)methyl acetate (prepared as reported for Example 55 & 56 steps 1-5; 160 mg, 0.370 mmol), Intermediate 12 (112.6 mg, 0.370 mmol) and DIPEA (391 uL, 2.2 mmol) in DMF (6.0 mL) was stirred at 85° C. for 5 h. After cooling, EtOAc (20 mL) was added and mixture washed with 5% citric acid (3 mL), H₂O (3 mL), brine (3 mL), dried over Na₂SO₄, and concentrated in vacuo to get the title compound as a brown solid (250 mg, 96%) which was used as crude. LCMS (ES⁺) Method 2: m/z 708 (M+H)⁺, RT 2.35 min

### Step 2: (3-((2-((tert-butoxycarbonyl)amino)-3-chloropyridin-4-yl)thio)-6-(7-(((tert-butoxycarbonyl)amino)methyl)-7-(5-methylisoxazol-3-yl)-3-azabicyclo[4.1.0]heptan-3-yl)-1-(tetrahydro-2H-pyran-2-yl)-1H-pyrazolo[3,4-b]pyrazin-5-yl)methyl acetate

A solution of (6-(7-(((tert-butoxycarbonyl)amino)methyl)-7-(5-methylisoxazol-3-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate (30 mg, 0.040 mmol), tert-butyl N-(3-chloranyl-4-sulfanyl-pyridin-2-yl)carbamate (12.2 mg, 0.050 mmol), Pd₂(dba)₃ (3.88 mg, 0.004 mmol), XantPhos (2.45 mg, 0.004 mmol) and DIPEA (10.9 mg, 0.080 mmol) in 1,4-dioxane (0.6 mL) was heated in a pressure tube at 85° C. for 1 h. After cooling mixture was concentrated under reduced pressure to afford the title compound as a brown oil (35 mg, 98%) which was used in the next step without further purification. LCMS (ES⁺) Method 2: m/z 840, 842 (M+H)⁺, RT 2.13 min

### Step 3: tert-butyl ((3-(3-((2-((tert-butoxycarbonyl)amino)-3-chloropyridin-4-yl)thio)-5-(hydroxymethyl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate

A solution of (3-((2-((tert-butoxycarbonyl)amino)-3-chloropyridin-4-yl)thio)-6-(7-(((tert-butoxycarbonyl)amino)methyl)-7-(5-methylisoxazol-3-yl)-3-azabicyclo[4.1.0]heptan-3-yl)-1-(tetrahydro-2H-pyran-2-yl)-1H-pyrazolo[3,4-b]pyrazin-5-yl)methyl acetate (35 mg, 0.040 mmol) in 1,4-dioxane (0.26 mL) was treated with a solution of LiOH (7.0 mg, 0.170 mmol) in H₂O (0.26 mL). The mixture was stirred at rt for 1 h. EtOAc and brine were added to the mixture and then the organic phase was isolated, dried over Na₂SO₄, and concentrated in vacuo to afford the title compound as a brown oil (33 mg, 99%) which was used in the next step without further purification. LCMS (ES⁺) Method 2: m/z 799 (M+H)⁺, RT 2.04 min

### Step 4: (3-((2-amino-3-chloropyridin-4-yl)thio)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol

A solution of tert-butyl ((3-(3-((2-((tert-butoxycarbonyl)amino)-3-chloropyridin-4-yl)thio)-5-(hydroxymethyl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (34 mg, 0.040 mmol) in DCM (0.70 mL) was treated with TFA (0.27 mL) and the mixture was stirred at rt for 5 min. The solvent was concentrated in vacuo and the residue was purified by preparative HPLC to obtain the title compound as a white solid (TFA salt; 1.2 mg, 5%). ¹H NMR (400 MHz, DMSO-d₆) δ 7.88 (br s, 3H), 7.59 (d, J=4.0 Hz, 1H), 6.56 (br s, 2H), 6.14 (s, 1H), 5.72 (d, J=8.0 Hz, 1H), 4.58 (s, 2H), 3.96-3.85 (m, 2H), 3.71-3.63 (m, 3H), 2.76-2.73 (m, 2H), 2.39 (s, 3H), 2.29-2.25 (m, 1H), 2.10-2.05 (m, 1H), 1.95-1.89 (m, 1H), 1.82-1.77 (m, 1H). LCMS (ES⁺) Method 2: m/z 514 (M+H)⁺, RT 0.83 min.

### Example 58: (3-(2-amino-3-chloropyridin-4-yl)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol (Trifluoro Acetate Salt)

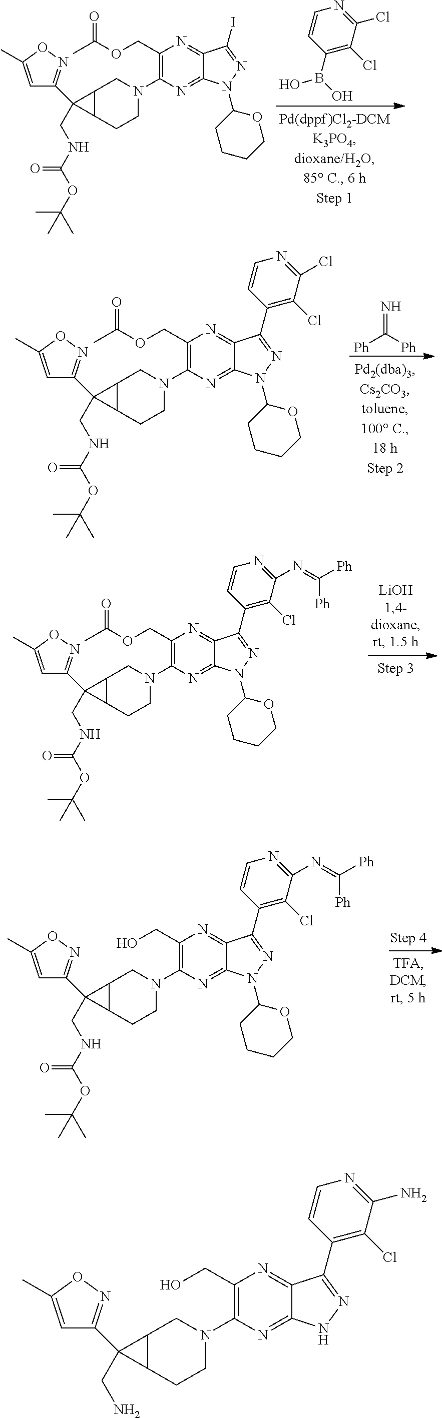

### Step 1: (6-(7-(((tert-butoxycarbonyl)amino)methyl)-7-(5-methylisoxazol-3-yl)-3-azabicyclo[4.1.0]heptan-3-yl)-3-(2,3-dichloropyridin-4-yl)-1-(tetrahydro-2H-pyran-2-yl)-1H-pyrazolo[3,4-b]pyrazin-5-yl)methyl acetate

(6-(7-(((tert-butoxycarbonyl)amino)methyl)-7-(5-methylisoxazol-3-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate (prepared as described in Example 57 step 1; 50 mg, 0.07 mmol), (2,3-dichloropyridin-4-yl)boronic acid (16 mg, 0.08 mmol), Pd(dppf)Cl₂·DCM (11 mg, 0.01 mmol), K₃PO₄ (90 mg, 0.42 mmol) were dissolved in a mixture of 1,4-dioxane (2.2 mL) and H₂O (0.4 mL). The mixture was degassed under N₂ and then heated at 85° C. for 6 h. After cooling down, EtOAc and H₂O were added to the mixture and the organic phase was separated, washed with brine, dried over Na₂SO₄, and concentrated in vacuo. The residue was purified by flash chromatography (0-100% EtOAc in petroleum ether) to afford the title compound as an orange oil (45 mg, 87%). LCMS (ES⁺) Method 2: m/z 727/729 (M+H)⁺, RT 2.55 min.

### Step 2: (6-(7-(((tert-butoxycarbonyl)amino)methyl)-7-(5-methylisoxazol-3-yl)-3-azabicyclo[4.1.0]heptan-3-yl)-3-(3-chloro-2-((diphenylmethylene)amino)pyridin-4-yl)-1-(tetrahydro-2H-pyran-2-yl)-1H-pyrazolo[3,4-b]pyrazin-5-yl)methyl acetate

A suspension of (6-(7-(((tert-butoxycarbonyl)amino)methyl)-7-(5-methylisoxazol-3-yl)-3-azabicyclo[4.1.0]heptan-3-yl)-3-(2,3-dichloropyridin-4-yl)-1-(tetrahydro-2H-pyran-2-yl)-1H-pyrazolo[3,4-b]pyrazin-5-yl)methyl acetate (45 mg, 0.060 mmol), [1-(2-diphenylphosphanyl-1-naphthyl)-2-naphthyl]-diphenyl-phosphane (7.7 mg, 0.012 mmol), diphenylmethanimine (16.8 mg, 0.093 mmol), Pd₂(dba)₃ (5.66 mg, 0.010 mmol), Cs₂CO₃ (60.3 mg, 0.190 mmol) in toluene (1.5 mL) was stirred at 100° C. for 18 h. After cooling down the mixture, DCM and H₂O were added and the organic phase was separated, washed with brine, dried over Na₂SO₄, and concentrated in vacuo to afford the title compound as a brown oil (53 mg) which was used without further purification. LCMS (ES⁺) Method 2: m/z 872, 873 (M+H)⁺, RT 2.41 min.

### Step 3: tert-butyl ((3-(3-(3-chloro-2-((diphenylmethylene)amino)pyridin-4-yl)-5-(hydroxymethyl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate

A solution of (6-(7-(((tert-butoxycarbonyl)amino)methyl)-7-(5-methylisoxazol-3-yl)-3-azabicyclo[4.1.0]heptan-3-yl)-3-(3-chloro-2-((diphenylmethylene)amino)pyridin-4-yl)-1-(tetrahydro-2H-pyran-2-yl)-1H-pyrazolo[3,4-b]pyrazin-5-yl)methyl acetate (53 mg, 0.06 mmol) in 1,4-dioxane (0.76 mL) was treated with a solution of LiOH (10 mg, 0.24 mmol) in H₂O (0.76 mL). The mixture was stirred at rt for 1.5 h. EtOAc and brine were added to the mixture and then the organic phase was isolated, dried over Na₂SO₄, and concentrated in vacuo to afford the title compound as a brown oil (50 mg) which was used in the next step without further purification. LCMS (ES⁺) Method 2: m/z 831 (M+H)⁺, RT 2.32 min.

### Step 4: (3-(2-amino-3-chloropyridin-4-yl)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol

A solution of tert-butyl ((3-(3-(3-chloro-2-((diphenylmethylene)amino)pyridin-4-yl)-5-(hydroxymethyl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (50 mg, 0.060 mmol) in DCM (1.0 mL) was treated with TFA (0.39 mL) and the mixture was stirred at rt for 5 h. The solvent was concentrated in vacuo and the residue was purified by flash chromatography (0-60% EtOAc in petroleum ether) to afford a residue which was dissolved in HCl in MeOH (1.25 M; 1.0 mL) and the mixture was stirred at rt for 18 h. After removal of the solvent under reduced pressure the residue was purified by preparative HPLC to obtain the title compound as a yellow solid (TFA salt; 0.61 mg, 2%). ¹H NMR (400 MHz, DMSO-d₆) δ 8.02 (d, J8.0 Hz, 1H), 7.89-7.87 (br s, 3H), 7.07 (d, J=8.0 Hz, 1H), 6.54 (br s, 2H), 6.14 (s, 1H), 4.62 (s, 2H), 3.94-3.88 (m, 2H), 3.70-3.66 (m, 3H), 2.75-2.71 (m, 2H), 2.40 (s, 3H), 2.33-2.26 (m, 1H), 2.10-2.05 (m, 1H), 1.95-1.89 (m, 1H), 1.82-1.77 (m, 1H). LCMS (ES⁺) Method 2: m/z 482 (M+H)⁺, RT 0.72 min.

### Example 59: 4-((6-(7-(aminomethyl)-7-(2-fluorophenyl)-3-azabicyclo[4.1.0]heptan-3-yl)-5H-pyrrolo[2,3-b]pyrazin-2-yl)thio)-3-chloropyridin-2-amine (Trifluoro Acetate Salt)

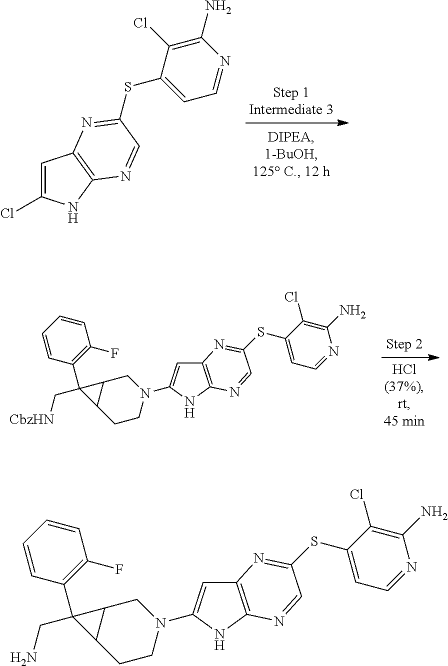 [SMILES: Nc1nccc(Sc2cnc3[nH]c(Cl)cc3n2)c1Cl ; Nc1nccc(Sc2cnc3[nH]c(N4CCC5C(C4)C5(CNC(=O)OCc4ccccc4)c4ccccc4F)cc3n2)c1Cl ; NCC1(c2ccccc2F)C2CCN(c3cc4nc(Sc5ccnc(N)c5Cl)cnc4[nH]3)CC21]

### Step 1: benzyl ((3-(2-((2-amino-3-chloropyridin-4-yl)thio)-5H-pyrrolo[2,3-b]pyrazin-6-yl)-7-(2-fluorophenyl)-3-azabicyclo[4.1.0]heptan-7-yl)methyl)carbamate

A solution of Intermediate 48 (11.5 mg, 0.040 mmol), Intermediate 3 (15.8 mg, 0.040 mmol) and DIPEA (0.03 mL, 0.150 mmol) in 1-BuOH (0.25 mL) was stirred at 125° C. for 12 h. After cooling the solvent was concentrated under reduced pressure, the residue was dissolved in EtOAc (1 mL), the organic phase was washed with H₂O, brine, dried over Na₂SO₄, and concentrated in vacuo to afford the title compound which was used without further purification. LCMS (ES⁺) Method 2: m/z 630 (M+H)⁺, RT 1.57 min.

### Step 2: 4-((6-(7-(aminomethyl)-7-(2-fluorophenyl)-3-azabicyclo[4.1.0]heptan-3-yl)-5H-pyrrolo[2,3-b]pyrazin-2-yl)thio)-3-chloropyridin-2-amine

Benzyl ((3-(2-((2-amino-3-chloropyridin-4-yl)thio)-5H-pyrrolo[2,3-b]pyrazin-6-yl)-7-(2-fluorophenyl)-3-azabicyclo[4.1.0]heptan-7-yl)methyl)carbamate (35 mg, 0.060 mmol) was treated with conc. HCl (37%; 0.8 mL) and the mixture was stirred at rt for 45 min. After removal of the solvent in vacuo the residue was purified by preparative HPLC to obtain the title compound as a yellow solid (TFA salt; 0.5 mg, 2%). LCMS (ES⁺) Method 2: m/z 496 (M+H)⁺, RT 0.92 min.

### Example 60: (3-(3-(3,4-dihydro-1,5-naphthyridin-1(2H)-yl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

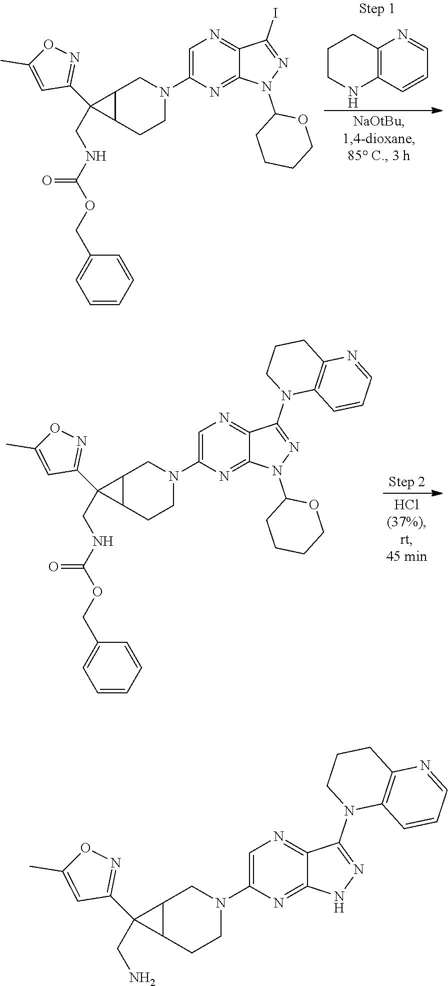 [SMILES: Cc1cc(C2(CNC(=O)OCc3ccccc3)C3CCN(c4cnc5c(I)nn(C6CCCCO6)c5n4)CC32)no1 ; c1cnc2c(c1)NCCC2 ; Cc1cc(C2(CNC(=O)OCc3ccccc3)C3CCN(c4cnc5c(N6CCCc7ncccc76)nn(C6CCCCO6)c5n4)CC32)no1 ; Cc1cc(C2(CN)C3CCN(c4cnc5c(N6CCCc7ncccc76)n[nH]c5n4)CC32)no1]

### Step 1: benzyl ((3-(3-(3,4-dihydro-1,5-naphthyridin-1(2H)-yl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate

A solution of benzyl ((3-(3-iodo-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (prepared with the procedure reported for Example 2 step 1 using Intermediate 6; 28.4 mg, 0.040 mmol), 1,2,3,4-tetrahydro-1,5-naphthyridine (4.61 mg, 0.030 mmol), sodium 2-methylpropan-2-olate (9.9 mg, 0.100 mmol), RuPhos (1.6 mg, 0003 mmol) and [2′-(methylamino)-2-biphenylyl]palladium methanesulfonate dicyclohexyl(2′,6′-diisopropoxy-2-biphenylyl)phosphine (2.9 mg, 0.003 mmol) in degassed 1,4-dioxane (1 mL) was heated in a sealed vial at 85° C. for 3 h. After cooling, the mixture was diluted with EtOAc (35 mL), washed with brine (2×5 mL), dried over Na₂SO₄, and concentrated in vacuo to afford the title compound as a yellow solid (21 mg, 90%) which was used without further purification. LCMS (ES⁺) Method 2: m/z 677 (M+H)⁺, RT 1.60 min.

### Step 2: (3-(3-(3,4-dihydro-1,5-naphthyridin-1(2H)-yl)-1H-pyrazolo[3,4-b]pyrazin-6-yl)-7-(5-methylisoxazol-3-yl)-3-azabicyclo[4.1.0]heptan-7-yl)methanamine

Benzyl ((3-(3-(3,4-dihydro-1,5-naphthyridin-1(2H)-yl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (20 mg, 0.030 mmol) was treated with conc. HCl (37%; 0.6 mL) and the mixture was stirred at rt for 16 h. After removal of the solvent in vacuo the residue was purified by preparative HPLC to obtain the title compound as a yellow solid (TFA salt; 1.0 mg, 7%). LCMS (ES⁺) Method 2: m/z 458 (M+H)⁺, RT 0.74 min

### Example 61: (3-(7-(aminomethyl)-3-(3-(4-chloro-2-methyl-2H-indazol-5-yl)-1H-pyrazolo[3,4-b]pyrazin-6-yl)-3-azabicyclo[4.1.0]heptan-7-yl)isoxazol-5-yl)methanol (Trifluoro Acetate Salt)

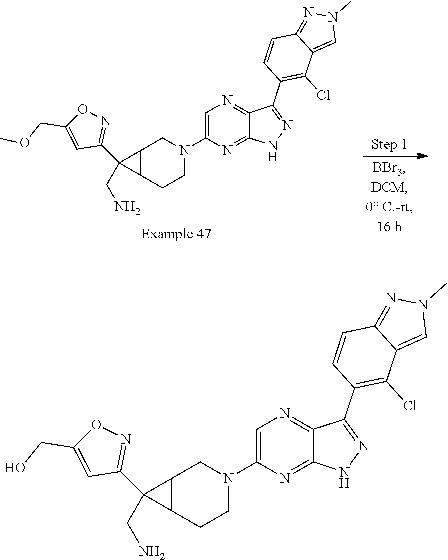

### Step 1: (3-(7-(aminomethyl)-3-(3-(4-chloro-2-methyl-2H-indazol-5-yl)-1H-pyrazolo[3,4-b]pyrazin-6-yl)-3-azabicyclo[4.1.0]heptan-7-yl)isoxazol-5-yl)methanol

A solution of (3-(3-(4-chloro-2-methyl-2H-indazol-5-yl)-1H-pyrazolo[3,4-b]pyrazin-6-yl)-7-(5-(methoxymethyl)isoxazol-3-yl)-3-azabicyclo[4.1.0]heptan-7-yl)methanamine (Example 47; 13 mg, 0.030 mmol) in DCM (1 mL) cooled at 0° C. was treated dropwise with boron tribromide (1M in hexanes, 75 uL, 0.08 mmol). The mixture was left warming to rt and stirred at this temperature for 16 h. MeOH (1 mL) was added to the mixture and solvent removal in vacuo the residue was purified by preparative HPLC to afford the title compound as a white solid (TFA salt; 0.4 mg, 3%). LCMS (ES⁺) Method 2: m/z 506 (M+H)⁺, RT 0.85 min.

### Example 62: 6-(7-(aminomethyl)-7-(5-methylisoxazol-3-yl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one (Trifluoro Acetate Salt)

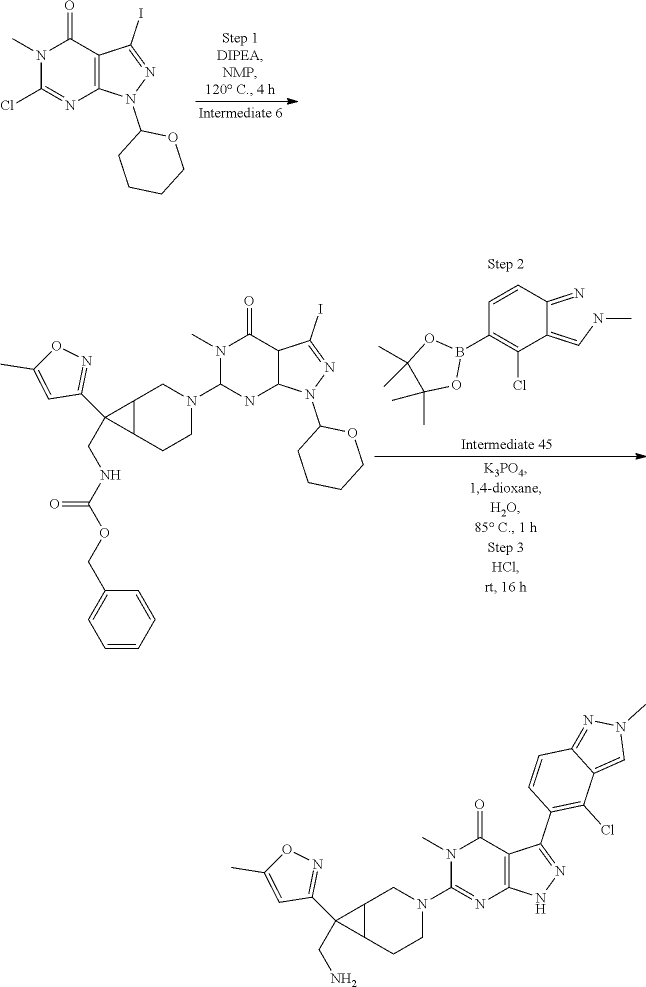

### Step 1: benzyl ((3-(3-iodo-5-methyl-4-oxo-1-(tetrahydro-2H-pyran-2-yl)-4,5-dihydro-1H-pyrazolo[3,4-d]pyrimidin-6-yl)-7-(5-methylisoxazol-3-yl)-3-azabicyclo[4.1.0]heptan-7-yl)methyl)carbamate

A solution of Intermediate 47 (100 mg, 0.250 mmol), Intermediate 6 (95.8 mg, 0.250 mmol), DIPEA (0.22 mL, 1.27 mmol) in NMP (2 mL) was stirred at 120° C. for 4 h. After cooling EtOAc (40 mL) was added and the mixture washed with H₂O, brine, dried over Na₂SO₄ and concentrated in vacuo to get a residue which was purified by flash chromatography (0-100% EtOAc in petroleum ether) to afford the title compound as a colourless oil. LCMS (ES⁺) Method 2: m/z 700 (M+H)⁺, RT 1.93 min.

### Step 2: benzyl ((3-(3-(4-chloro-2-methyl-2H-indazol-5-yl)-5-methyl-4-oxo-1-(tetrahydro-2H-pyran-2-yl)-4,5-dihydro-1H-pyrazolo[3,4-d]pyrimidin-6-yl)-7-(5-methylisoxazol-3-yl)-3-azabicyclo[4.1.0]heptan-7-yl)methyl)carbamate

A solution of benzyl ((3-(3-iodo-5-methyl-4-oxo-1-(tetrahydro-2H-pyran-2-yl)-4,5-dihydro-1H-pyrazolo[3,4-d]pyrimidin-6-yl)-7-(5-methylisoxazol-3-yl)-3-azabicyclo[4.1.0]heptan-7-yl)methyl)carbamate (77 mg, 0.060 mmol), Intermediate 45 (20.9 mg, 0.070 mmol), Pd(dppf)Cl₂·DCM (9.1 mg, 0.010 mmol) and K₃PO₄ (70.1 mg, 0.330 mmol) in a mixture of degassed 1,4-dioxane (1 mL) and H₂O (0.1 mL) was heated at 85° C. for 1 h. After cooling, the mixture was diluted with EtOAc (35 mL), washed with brine, dried over Na₂SO₄ and concentrated in vacuo to afford a residue which was purified by flash chromatography (0-100% EtOAc in petroleum ether) to afford the title compound as a yellow oil (25 mg, 62%). LCMS (ES⁺) Method 2: m/z 738 (M+H)⁺, RT 1.92 min.

### Step 3: 6-(7-(aminomethyl)-7-(5-methylisoxazol-3-yl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one

Benzyl ((3-(3-(4-chloro-2-methyl-2H-indazol-5-yl)-5-methyl-4-oxo-1-(tetrahydro-2H-pyran-2-yl)-4,5-dihydro-1H-pyrazolo[3,4-d]pyrimidin-6-yl)-7-(5-methylisoxazol-3-yl)-3-azabicyclo[4.1.0]heptan-7-yl)methyl)carbamate (22 mg, 0.030 mmol) was treated with conc. HCl (37%; 0.6 mL, 0.030 mmol) and the mixture was stirred at rt for 16 h. After volatiles removal in vacuo the residue was purified by preparative HPLC to obtain the title compound as a white solid (TFA salt; 5 mg, 32%). ¹H NMR (400 MHz, DMSO-d₆) δ 13.55 (br s, 1H), 8.53 (s, 1H), 7.96 (br s, 3H), 7.61 (d, J=8.0 Hz, 1H), 7.31 (d, J=8.0 Hz, 1H), 6.12 (s, 1H), 4.23 (s, 3H), 3.71-3.65 (m, 5H), 3.34 (s, 3H), 2.67-2.58 (m, 1H), 2.39 (s, 3H), 2.33-2.26 (m, 1H), 1.99-1.95 (m, 1H), 1.89-1.87 (m, 1H), 1.77-1.74 (m, 1H). LCMS (ES⁺) Method 2: m/z 520 (M+H)⁺, RT 0.87 min.

### Example 63: (7-phenyl-3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

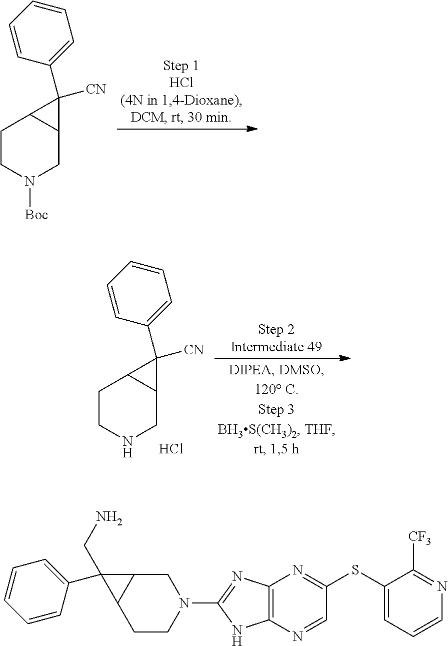 [SMILES: CC(C)(C)OC(=O)N1CCC2C(C1)C2(C#N)c1ccccc1 ; N#CC1(c2ccccc2)C2CCNCC21 ; Cl ; NCC1(c2ccccc2)C2CCN(c3nc4nc(Sc5cccnc5C(F)(F)F)cnc4[nH]3)CC21]

### Step 1: 7-phenyl-3-azabicyclo[4.1.0]heptane-7-carbonitrile

Tert-butyl 7-cyano-7-phenyl-3-azabicyclo[4.1.0]heptane-3-carboxylate (prepared as described in Intermediate 28 step 1; 50 mg, 0.17 mmol) was dissolved in DCM (1 mL) and treated with 1 mL of 4N HCl in 1,4-dioxane. The solution was stirred at rt for 1.5 h, then solvent was removed in vacuo to give the title compound (44 mg; 99%). LCMS (ES⁺) Method 1A: m/z 199 (M+H)⁺, RT 1.04 min.

### Step 2: 7-phenyl-3-(5-((2-(trifluoromethyl)pyridin-3-yl)thio)-1H-imidazo[4,5-b]pyrazin-2-yl)-3-azabicyclo[4.1.0]heptane-7-carbonitrile

Dry DIPEA (0.12 mL, 0.67 mmol) was added to a solution of Intermediate 49 (56 mg, 0.17 mmol) and 7-phenyl-3-azabicyclo[4.1.0]heptane-7-carbonitrile (39 mg, 0.17 mmol) in DMSO (1.3 mL). The reaction mixture was stirred at 120° C. for 2 h. The reaction was diluted with EtOAc (×2) and washed with H₂O (×2), 5% citric acid solution (×2), NaHCO₃ sat. sol. (×2). Organic layer was dried over Na₂SO₄, filtered and concentrated in vacuo. The resulting crude was purified by flash chromatography (80-100% EtOAc in petroleum ether) to obtain the title compound as light-yellow solid (60 mg, 72%). LCMS (ES⁺) Method 1A: m/z 494 (M+H)⁺, RT 1.94 min.

### Step 3: (7-phenyl-3-(5-((2-(trifluoromethyl)pyridin-3-yl)thio)-1H-imidazo[4,5-b]pyrazin-2-yl)-3-azabicyclo[4.1.0]heptan-7-yl)methanamine

7-Phenyl-3-(5-((2-(trifluoromethyl)pyridin-3-yl)thio)-1H-imidazo[4,5-b]pyrazin-2-yl)-3-azabicyclo[4.1.0]heptane-7-carbonitrile (46 mg, 0.09 mmol) was dissolved in dry THF (0.5 mL) and BMS (2M in THF; 0.16 mL, 0.33 mmol) was added at rt under N₂ atmosphere. The reaction mixture was stirred at rt for 1 h and 40 min then additional BMS (2M in THF; 0.2 mL, 0.40 mmol) was added and the resulting solution was stirred at rt for 12 h. The reaction was quenched by addition of MeOH (2 mL) and concentrated in vacuo. The residue was dissolved in THF (0.5 mL), 6N HCl aqueous solution (2 mL) was added and the mixture was stirred at rt for 3.5 h. Volatiles were removed in vacuo and the residue was purified by preparative HPLC (10-60% MeCN/H₂O+0.1% TFA) to obtain the title compound as a light-yellow powder (TFA salt; 9.5 mg, 16%). ¹H NMR (300 MHz, DMSO-d₆+TFA) δ 8.61 (br d, J=4.6 Hz, 1H), 8.24 (s, 1H), 7.84 (d, J=8.1 Hz, 1H), 7.75-7.57 (m, 4H), 7.45 (m, 2H), 7.37 (m, 2H), 7.29 (m, 1H), 4.22 (br d, J=14.4 Hz, 1H), 4.02-3.84 (m, 2H), 3.23 (m, 1H), 3.18-2.99 (m, 2H), 2.34 (m, 1H), 1.87-1.63 (m, 2H), 1.52 (m, 1H); LCMS (ES⁺) Method 2: m/z 498 (M+H)⁺, RT 2.38 min.

### Example 64: (7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

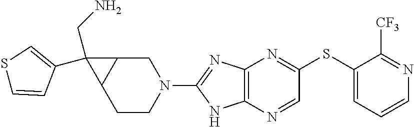 [SMILES: NCC1(c2ccsc2)C2CCN(c3nc4nc(Sc5cccnc5C(F)(F)F)cnc4[nH]3)CC21]

### Step 1: benzyl ((7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate

Dry DIPEA (0.01 mL, 0.06 mmol) was added to a solution of Intermediate 49 (4.5 mg, 0.01 mmol) and Intermediate 31 (4.3 mg, 0.01 mmol) in DMSO (0.2 mL). The reaction mixture was stirred at 120° C. for 3 h. The reaction was directly purified by preparative HPLC (20-75% MeCN/H₂O+0.1% HCOOH) to obtain the title compound as a beige solid (5 mg, 69%). LCMS (ES⁺) Method 1A: m/z 638 (M+H)⁺, RT 2.13 min.

### Step 2: (7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine

Benzyl ((7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate (5 mg, 0.01 mmol) was dissolved in conc. HCl (37%; 0.5 mL) and the resulting yellow solution was stirred at rt for 12 h. The reaction was directly purified by preparative HPLC (5-45% MeCN/H₂O+0.1% TFA) to obtain the title compound as an off-white solid (TFA salt; 4 mg, 81%). ¹H NMR (300 MHz, DMSO-d₆+TFA) δ 8.61 (br d, J=4.6 Hz, 1H), 8.25 (s, 1H), 7.84 (d, J=8.2 Hz, 1H), 7.75 (br s, 3H), 7.62 (m, 1H), 7.54 (m, 1H), 7.41 (br s, 1H), 7.13 (d, J=4.8 Hz, 1H), 4.11 (d, J=14.2 Hz, 1H), 3.99-3.84 (m, 2H), 3.32 (m, 1H), 3.20-2.99 (m, 2H), 2.31 (m, 1H), 1.86 (m, 1H), 1.68 (m, 1H), 1.54 (m, 1H); LCMS (ES⁺) Method 2: m/z 504 (M+H)⁺, RT 2.34 min.

### Example 65: (7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

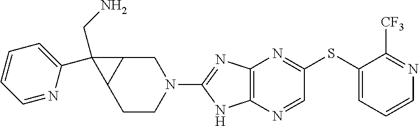 [SMILES: NCC1(c2ccccn2)C2CCN(c3nc4nc(Sc5cccnc5C(F)(F)F)cnc4[nH]3)CC21]

### Step 1: benzyl ((7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate

Benzyl ((7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate was prepared according to the procedure described in the synthesis of Example 64 step 1 starting from Intermediate 29 (16.4 mg, 0.04 mmol). The reaction was purified by preparative HPLC (5-50% MeCN/H₂O+0.1% TFA) to obtain the title compound as a beige solid (16 mg, 63%). ¹H NMR (300 MHz, DMSO-d₆+TFA) δ 8.83 (br d, J=5.6 Hz, 1H), 8.60 (m, 1H), 8.43 (dt, J=7.9, 1.5 Hz, 1H), 8.27 (s, 1H), 8.01-7.80 (m, 3H), 7.60 (m, 1H), 7.42 (m, 1H), 7.37-7.24 (m, 3H), 7.15 (m, 2H), 4.80 (s, 2H), 4.30 (d, J=4.3 Hz, 1H), 4.05-3.83 (m, 2H), 3.60 (m, 1H), 3.36 (m, 1H), 3.15 (m, 1H), 2.28 (m, 1H), 2.07-1.90 (m, 2H), 1.82 (m, 1H); LCMS (ES⁺) Method 1A: m/z 633 (M+H)⁺, RT 1.58 min.

### Step 2: (7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine

(7-(Pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine was prepared according to the procedure described in the synthesis of Example 64 step 2 starting from benzyl ((7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate (15 mg, 0.02 mmol). The reaction was purified by preparative HPLC (5-45% MeCN/H₂O+0.1% TFA) to obtain the title compound as a light-yellow solid (TFA salt; 10 mg, 69%). ¹H NMR (300 MHz, DMSO-d₆+TFA) δ 8.74 (br d, J=5.3 Hz, 1H), 8.60 (m, 1H), 8.26-8.15 (m, 2H), 7.96-7.76 (m, 4H), 7.70-7.56 (m, 3H), 4.29 (d, J=14.7 Hz, 1H), 4.00 (m, 1H), 3.88 (m, 1H), 3.45 (m, 1H), 3.28-3.04 (m, 2H), 2.33 (m, 1H), 2.12 (m, 1H), 1.92-1.68 (m, 2H); LCMS (ES⁺) Method 2. m/z 499 (M+H)⁺, RT 2.05 min.

### Example 66: (7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

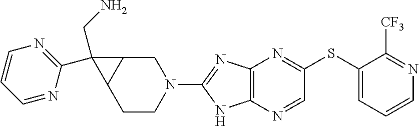 [SMILES: NCC1(c2ncccn2)C2CCN(c3nc4nc(Sc5cccnc5C(F)(F)F)cnc4[nH]3)CC21]

### Step 1: benzyl ((7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate

Benzyl ((7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate was prepared according to the procedure described in the synthesis of Example 64 step 1 starting from Intermediate 30 (15 mg, 0.04 mmol). The reaction was purified by preparative HPLC (20-65% MeCN/H₂O+0.1% TFA) to obtain the title compound as a yellow solid (22 mg, 87%). ¹H NMR (300 MHz, DMSO-d₆+TFA) δ 8.70 (d, J=4.9 Hz, 2H), 8.61 (m, 1H), 8.33 (s, 1H), 7.90 (d, J=8.2 Hz, 1H), 7.61 (m, 1H), 7.38-7.21 (m, 6H), 7.05 (m, 1H), 4.84 (m, 2H), 4.15-3.89 (m, 3H), 3.70 (m, 2H), 3.23 (m, 1H), 2.28 (m, 1H), 2.14 (m, 1H), 2.00 (m, 1H), 1.92-1.74 (m, 1H); LCMS (ES⁺) Method 1A m/z 634 (M+H)⁺, RT 2.00 min.

### Step 2: (7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine

(7-(Pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine was prepared according to the procedure described in the synthesis of Example 64 step 2 starting from benzyl ((7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate (21 mg, 0.03 mmol). The reaction was purified by flash chromatography (C18; 5-40% MeCN/H₂O+0.1% TFA) to obtain the title compound as a light-yellow solid (TFA salt; 15 mg, 74%). ¹H NMR (300 MHz, DMSO-d₆+TFA) δ 8.76 (d, J=4.9 Hz, 2H), 8.60 (m, 1H), 8.22 (s, 1H), 7.90 (br s, 3H), 7.81 (m, 1H), 7.62 (m, 1H), 7.39 (t, J=4.9 Hz, 1H), 4.22 (d, J=4.5 Hz, 1H), 4.04 (m, 1H), 3.91 (m, 1H), 3.55 (m, 1H), 3.31-3.08 (m, 2H), 2.40-2.20 (m, 1H), 2.04 (m, 1H), 1.78 (m, 1H); LCMS (ES⁺) Method 2: m/z 500 (M+H)⁺, RT 2.43 min.

### Example 67: (7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

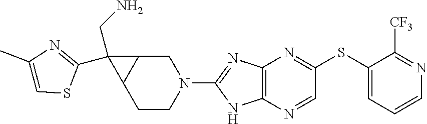 [SMILES: Cc1csc(C2(CN)C3CCN(c4nc5nc(Sc6cccnc6C(F)(F)F)cnc5[nH]4)CC32)n1]

### Step 1: benzyl ((7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate

Benzyl ((7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate was prepared according to the procedure described in the synthesis of Example 64 step 1 starting from Intermediate 32 (18 mg, 0.05 mmol). The reaction was purified by preparative HPLC (20-65% MeCN/H₂O+0.1% TFA) to obtain the title compound as a beige solid (19 mg, 63%). ¹H NMR (300 MHz, DMSO-d₆+TFA) δ 8.61 (br d, J=4.6 Hz, 1H), 8.30 (s, 1H), 7.88 (d, J=8.1 Hz, 1H), 7.61 (m, 1H), 7.45 (m, 1H), 7.38-7.22 (m, 5H), 7.13 (s, 1H), 4.92 (s, 2H), 4.10 (d, J=14.3 Hz, 1H), 4.02-3.82 (m, 2H), 3.68 (m, 1H), 3.45 (m, 1H), 3.16 (m, 1H), 2.30 (m, 4H), 2.07 (m, 1H), 1.94 (m, 1H), 1.81 (m, 1H); LCMS (ES⁺) Method 1A: m/z 653 (M+H)⁺, RT 1.98 min.

### Step 2: (7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine

(7-(4-Methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine was prepared according to the procedure described in the synthesis of Example 64 step 2 starting from benzyl ((7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methyl)carbamate (17 mg, 0.03 mmol). The reaction was purified by preparative HPLC (5-50% MeCN/H₂O+0.1% TFA) to obtain the title compound as a light-yellow solid (TFA salt; 11 mg, 68%). ¹H NMR (300 MHz, DMSO-d₆+TFA) δ 8.61 (m, 1H), 8.23 (s, 1H), 7.95 (br s, 3H), 7.83 (m, 1H), 7.62 (m, 1H), 7.18 (br d, J=1.0 Hz, 1H), 4.18 (d, J=14.6 Hz, 1H), 4.05-3.83 (m, 2H), 3.49 (m, 1H), 3.32-3.03 (m, 2H), 2.36 (m, 4H), 2.02 (m, 1H), 1.88-1.67 (m, 2H); LCMS (ES⁺) Method 2: m/z 519 (M+H)⁺, RT 2.33 min.

### Example 68: (3-(5-((2-(trifluoromethyl)pyridin-3-yl)thio)-1H-imidazo[4,5-b]pyrazin-2-yl)-3-azabicyclo[4.1.0]heptan-7-yl)methanamine (Trifluoro Acetate Salt)

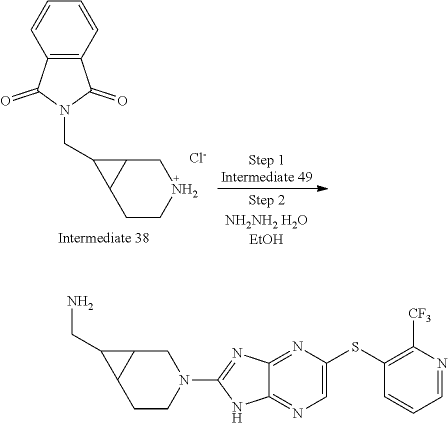 [SMILES: O=C1c2ccccc2C(=O)N1CC1C2CCNCC21 ; [Cl-] ; NCC1C2CCN(c3nc4nc(Sc5cccnc5C(F)(F)F)cnc4[nH]3)CC12]

### Step 1: 2-((3-(5-((2-(trifluoromethyl)pyridin-3-yl)thio)-1H-imidazo[4,5-b]pyrazin-2-yl)-3-azabicyclo[4.1.0]heptan-7-yl)methyl)isoindoline-1,3-dione

2-((3-(5-((2-(Trifluoromethyl)pyridin-3-yl)thio)-1H-imidazo[4,5-b]pyrazin-2-yl)-3-azabicyclo[4.1.0]heptan-7-yl)methyl)isoindoline-1,3-dione was prepared according to the procedure described in the synthesis of Example 64 step 1 starting from Intermediate 38 (21 mg, 0.07 mmol). The reaction was purified by preparative HPLC (0-45% MeCN/H₂O+0.1% TFA) to afford the title compound as a colorless oil (27 mg, 81%). LCMS (ES⁺) Method 1A: m/z 552 (M+H)⁺, RT 1.90 min.

### Step 2: (3-(5-((2-(trifluoromethyl)pyridin-3-yl)thio)-1H-imidazo[4,5-b]pyrazin-2-yl)-3-azabicyclo[4.1.0]heptan-7-yl)methanamine

To a solution 2-((3-(5-((2-(trifluoromethyl)pyridin-3-yl)thio)-1H-imidazo[4,5-b]pyrazin-2-yl)-3-azabicyclo[4.1.0]heptan-7-yl)methyl)isoindoline-1,3-dione (27 mg, 0.05 mmol) in EtOH (2 mL) N₂H₄·H₂O (27 uL, 0.54 mmol) was added. The resulting mixture was stirred at rt for 3 h. Volatiles were removed in vacuo and the crude was purified by preparative HPLC (5-45% MeCN/H₂O+0.1% TFA) to afford pure desired compound (TFA salt; 20 mg, 96%). ¹H NMR (300 MHz, DMSO-d₆+TFA) δ 8.61-8.65 (m, 1H), 8.30 (s, 1H), 7.89 (d, J=8.0 Hz, 1H), 7.74 (br s, 3H), 7.63 (dd, J=8.1, 4.5 Hz, 1H), 4.03-3.93 (m, 1H), 3.90-3.81 (m, 1H), 3.57-3.34 (m, 2H), 2.80-2.71 (m, 2H), 2.20-2.08 (m, 1H), 1.85-1.73 (m, 1H), 1.31-1.15 (m, 2H), 1.04-0.89 (m, 1H); LCMS (ES⁺) Method 2: m/z 422 (M+H)⁺, RT 2.06 min.

### Example 69: 4-((2-(7-(aminomethyl)-7-phenyl-3-azabicyclo[4.1.0]heptan-3-yl)-1H-imidazo[4,5-b]pyrazin-5-yl)thio)-3-chloropyridin-2-amine (Trifluoro Acetate Salt)

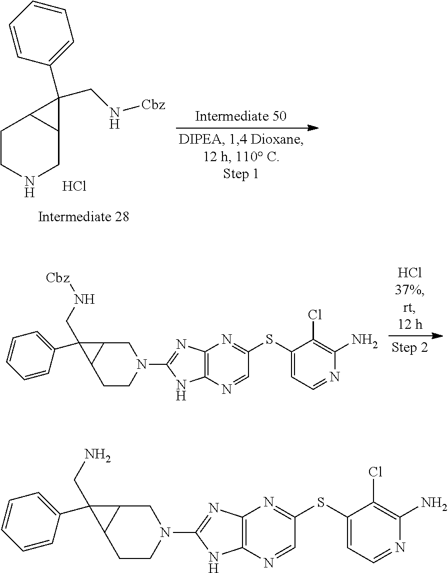 [SMILES: O=C(NCC1(c2ccccc2)C2CCNCC21)OCc1ccccc1 ; Cl ; Nc1nccc(Sc2cnc3[nH]c(N4CCC5C(C4)C5(CNC(=O)OCc4ccccc4)c4ccccc4)nc3n2)c1Cl ; NCC1(c2ccccc2)C2CCN(c3nc4nc(Sc5ccnc(N)c5Cl)cnc4[nH]3)CC21]

### Step 1: benzyl ((3-(5-((2-amino-3-chloropyridin-4-yl)thio)-1H-imidazo[4,5-b]pyrazin-2-yl)-7-phenyl-3-azabicyclo[4.1.0]heptan-7-yl)methyl)carbamate

A mixture of Intermediate 28 (20 mg, 0.06 mmol), Intermediate 50 (20 mg, 0.06 mmol) and DIPEA (50 uL, 0.3 mmol) in 1,4-dioxane was stirred in a sealed tube at 120° C. for 24 h. The reaction was purified by preparative HPLC (10-65% MeCN/H₂O+0.1% TFA) affording the desired compound as a colorless oil. LCMS (ES⁺) Method 1A: m/z 613, 615 (M+H)⁺, RT 1.66 min.

### Step 2: 4-((2-(7-(aminomethyl)-7-phenyl-3-azabicyclo[4.1.0]heptan-3-yl)-1H-imidazo[4,5-b]pyrazin-5-yl)thio)-3-chloropyridin-2-amine

The compound was prepared according to the procedure described in the synthesis of Example 64 step 2 starting from benzyl ((7-phenyl-3-azabicyclo[4.1.0]heptan-7-yl)methyl)carbamate. The reaction was purified by preparative HPLC (0-45% MeCN/H₂O+0.1% TFA) to obtain the title compound as a light-yellow solid (TFA salt; 2 mg, 7% over two steps). ¹H NMR (300 MHz, DMSO-d₆+TFA) δ 8.32 (s, 1H), 7.76-7.66 (m, 4H), 7.49-7.44 (m, 2H), 7.37 (t, J=7.4 Hz, 2H), 7.32-7.25 (m, 1H), 6.31-6.18 (m, 1H), 4.39-4.21 (m, 1H), 4.06-3.89 (m, 2H), 3.31-3.04 (m, 3H), 2.43-2.32 (m, 1H), 1.92-1.68 (m, 2H), 1.6-1.48 (m, 1H); LCMS (ES⁺) Method 2: m/z 479, 481 (M+H)⁺, RT 1.74 min.

### Example 70: 4-((2-(7-(aminomethyl)-7-(4-methylthiazol-2-yl)-3-azabicyclo[4.1.0]heptan-3-yl)-1H-imidazo[4,5-b]pyrazin-5-yl)thio)-3-chloropyridin-2-amine (Trifluoro Acetate Salt)

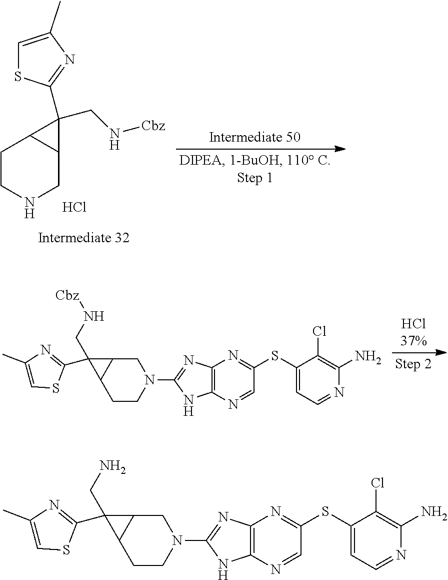 [SMILES: Cc1csc(C2(CNC(=O)OCc3ccccc3)C3CCNCC32)n1 ; Cl ; Cc1csc(C2(CNC(=O)OCc3ccccc3)C3CCN(c4nc5nc(Sc6ccnc(N)c6Cl)cnc5[nH]4)CC32)n1 ; Cc1csc(C2(CN)C3CCN(c4nc5nc(Sc6ccnc(N)c6Cl)cnc5[nH]4)CC32)n1]

### Step 1: benzyl ((3-(5-((2-amino-3-chloropyridin-4-yl)thio)-1H-imidazo[4,5-b]pyrazin-2-yl)-7-(4-methylthiazol-2-yl)-3-azabicyclo[4.1.0]heptan-7-yl)methyl)carbamate

To a solution of Intermediate 50 (21 mg, 0.06 mmol) and Intermediate 32 (25 mg, 0.06 mmol) in 1-butanol (0.7 mL), dry DIPEA (0.05 mL, 0.29 mmol) was added. The reaction mixture was stirred at 110° C. for 23 h, then concentrated in vacuo. The resulting crude was purified by flash chromatography (C18; 5-45% MeCN/H₂O+0.1% TFA) to obtain the title compound as a yellow solid (13 mg, 35%). LCMS (ES⁺) Method 1A: m/z 634 (M+H)⁺, RT 1.48 min.

### Step 2: 4-((2-(7-(aminomethyl)-7-(4-methylthiazol-2-yl)-3-azabicyclo[4.1.0]heptan-3-yl)-1H-imidazo[4,5-b]pyrazin-5-yl)thio)-3-chloropyridin-2-amine

The compound was prepared according to the procedure described in the synthesis of Example 64 step 2 starting from benzyl ((3-(5-((2-amino-3-chloropyridin-4-yl)thio)-1H-imidazo[4,5-b]pyrazin-2-yl)-7-(4-methylthiazol-2-yl)-3-azabicyclo[4.1.0]heptan-7-yl)methyl)carbamate (13 mg, 0.02 mmol). The reaction was purified by flash chromatography (C18; 0-30% MeCN/H₂O+0.1% TFA) to obtain the title compound as an off-white solid (TFA salt; 6 mg, 49%). ¹H NMR (300 MHz, DMSO-d₆+TFA) δ 8.30 (s, 1H), 8.00 (br s, 3H) 7.73 (d, J=6.9 Hz, 1H), 7.19 (br d, J=1.0 Hz, 1H), 6.23 (d, J=6.9 Hz, 1H), 4.28 (d, J=14.6 Hz, 1H), 4.05 (m, 1H), 3.94 (m, 1H), 3.50 (m, 1H), 3.33-3.07 (m, 2H), 2.37 (m, 4H), 2.03 (m, 1H), 1.89-1.72 (m, 2H); LCMS (ES⁺) Method 2: m/z 500 (M+H)⁺, RT 1.66 min.

### Example 71: 6-(7-(aminomethyl)-7-phenyl-3-azabicyclo[4.1.0]heptan-3-yl)-3-((2-(trifluoromethyl)pyridin-3-yl)thio)pyrazin-2-amine (Trifluoro Acetate Salt)

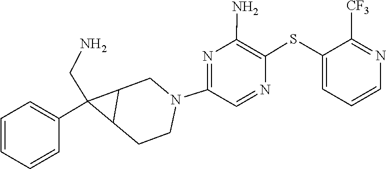 [SMILES: NCC1(c2ccccc2)C2CCN(c3cnc(Sc4cccnc4C(F)(F)F)c(N)n3)CC21]

### Step 1: benzyl ((3-(6-amino-5-((2-(trifluoromethyl)pyridin-3-yl)thio)pyrazin-2-yl)-7-phenyl-3-azabicyclo[4.1.0]heptan-7-yl)methyl)carbamate

Benzyl ((3-(6-amino-5-((2-(trifluoromethyl)pyridin-3-yl)thio)pyrazin-2-yl)-7-phenyl-3-azabicyclo[4.1.0]heptan-7-yl)methyl)carbamate was prepared by reacting Intermediate 28 (32 mg, 0.09 mmol) and Intermediate 41 (20 mg, 0.06 mmol) under the same conditions described in the synthesis of Example 64 step 1. The reaction was purified by preparative HPLC (15-60% MeCN/H₂O+0.1% HCOOH) to obtain the title compound as a brown solid (20 mg, 50%). ¹H NMR (300 MHz, DMSO-d₆) δ 8.45 (d, J=4.3 Hz, 1H), 7.62-7.56 (m, 1H), 7.55-7.47 (m, 1H), 7.41-7.01 (m, 13H), 6.20 (br s, 1H), 4.87 (s, 2H), 4.31-4.24 (m, 2H), 3.70-3.62 (m, 1H), 3.46-3.28 (m, 2H), 2.83-2.74 (m, 1H), 2.25-2.10 (m, 1H), 1.87-1.70 (m, 1H), 1.69-1.58 (m, 1H), 1.43-1.33 (m, 1H); LCMS (ES⁺) Method 1A: m/z 607 (M+H)⁺, RT 2.47 min.

### Step 2: 6-(7-(Aminomethyl)-7-phenyl-3-azabicyclo[4.1.0]heptan-3-yl)-3-((2-(trifluoromethyl)pyridin-3-yl)thio)pyrazin-2-amine

6-(7-(Aminomethyl)-7-phenyl-3-azabicyclo[4.1.0]heptan-3-yl)-3-((2-(trifluoromethyl)pyridin-3-yl)thio)pyrazin-2-amine was prepared from benzyl ((3-(6-amino-5-((2-(trifluoromethyl)pyridin-3-yl)thio)pyrazin-2-yl)-7-phenyl-3-azabicyclo[4.1.0]heptan-7-yl)methyl)carbamate (20 mg, 0.03 mmol) according to the procedure described in the synthesis of Example 64 step 2. The reaction was purified by preparative HPLC (5-45% MeCN/H₂O+0.1% TFA) to obtain the title compound as a brown solid (TFA salt; 7.36 mg, 47%). ¹H NMR (300 MHz, DMSO-d₆+TFA) δ 8.47 (br d, J=4.4 Hz, 1H) 7.70-7.59 (m, 4H), 7.57-7.42 (m, 3H), 7.41-7.25 (m, 4H), 4.34-4.19 (m, 2H), 3.63 (br dd, J=14.2, 6.3 Hz, 1H), 3.31-3.20 (m, 1H), 3.17-3.06 (m, 1H), 2.66-2.84 (m, 1H), 2.36-2.18 (m, 1H), 1.85-1.61 (m, 2H), 1.52-1.42 (m, 1H); LCMS (ES⁺) Method 2: m/z 473 (M+H)⁺, RT 2.60 min.

### Example 72: 6-(7-(aminomethyl)-7-(pyridin-2-yl)-3-azabicyclo[4.1.0]heptan-3-yl)-3-((2-(trifluoromethyl)pyridin-3-yl)thio)pyrazin-2-amine (Trifluoro Acetate Salt)

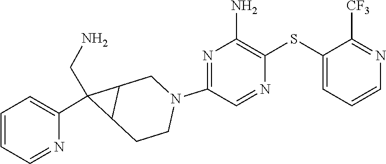 [SMILES: NCC1(c2ccccn2)C2CCN(c3cnc(Sc4cccnc4C(F)(F)F)c(N)n3)CC21]

### Step 1: benzyl ((3-(6-amino-5-((2-(trifluoromethyl)pyridin-3-yl)thio)pyrazin-2-yl)-7-(pyridin-2-yl)-3-azabicyclo[4.1.0]heptan-7-yl)methyl)carbamate

Benzyl ((3-(6-amino-5-((2-(trifluoromethyl)pyridin-3-yl)thio)pyrazin-2-yl)-7-(pyridin-2-yl)-3-azabicyclo[4.1.0]heptan-7-yl)methyl)carbamate was prepared by reacting Intermediate 29 (10 mg, 0.024 mmol) and Intermediate 41 (10 mg, 0.03 mmol) under the same conditions described in the synthesis of Example 64 step 1. The reaction was purified by preparative HPLC (15-60% MeCN/H₂O+0.1% TFA) to obtain the title compound as a brown solid (6 mg, 41%). Method 1A: m/z 608 (M+H)⁺, RT 1.66 min.

### Step 2: 6-(7-(aminomethyl)-7-(pyridin-2-yl)-3-azabicyclo[4.1.0]heptan-3-yl)-3-((2-(trifluoromethyl)pyridin-3-yl)thio)pyrazin-2-amine

6-(7-(Aminomethyl)-7-(pyridin-2-yl)-3-azabicyclo[4.1.0]heptan-3-yl)-3-((2-(trifluoromethyl)pyridin-3-yl)thio)pyrazin-2-amine was prepared from benzyl ((3-(6-amino-5-((2-(trifluoromethyl)pyridin-3-yl)thio)pyrazin-2-yl)-7-(pyridin-2-yl)-3-azabicyclo[4.1.0]heptan-7-yl)methyl)carbamate (6 mg, 0.01 mmol) according to the procedure described in the synthesis of Example 64 step 2. The reaction was purified by preparative HPLC (5-45% MeCN/H₂O+0.1% TFA) to afford the title compound as a brown solid (TFA salt; 4 mg, 69%). ¹H NMR (300 MHz, DMSO-d₆+TFA) δ 8.74 (br d, J=5.1 Hz, 1H), 8.48 (br d, J=4.7 Hz, 1H), 8.18 (m, 1H), 7.88 (br s, 3H), 7.70-7.59 (m, 3H), 7.55 (m, 1H), 7.33 (d, J=8.2 Hz, 1H), 4.37-4.20 (m, 2H), 3.61 (m, 1H), 3.47 (m, 1H), 3.26 (m, 1H), 2.81 (m, 1H), 2.29 (m, 1H), 2.09 (m, 1H), 1.86-1.63 (m, 2H); LCMS (ES⁺) Method 2 m/z 474 (M+H)⁺, RT 2.12 min.

### Example 73: 6-(7-(aminomethyl)-7-(4-methylthiazol-2-yl)-3-azabicyclo[4.1.0]heptan-3-yl)-3-((2-(trifluoromethyl)pyridin-3-yl)thio)pyrazin-2-amine (Trifluoro Acetate Salt)

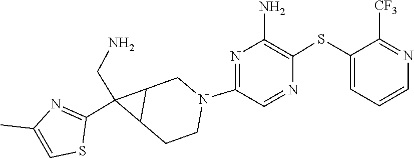 [SMILES: Cc1csc(C2(CN)C3CCN(c4cnc(Sc5cccnc5C(F)(F)F)c(N)n4)CC32)n1]

### Step 1: benzyl ((3-(6-amino-5-((2-(trifluoromethyl)pyridin-3-yl)thio)pyrazin-2-yl)-7-(4-methylthiazol-2-yl)-3-azabicyclo[4.1.0]heptan-7-yl)methyl)carbamate

Benzyl ((3-(6-amino-5-((2-(trifluoromethyl)pyridin-3-yl)thio)pyrazin-2-yl)-7-(4-methylthiazol-2-yl)-3-azabicyclo[4.1.0]heptan-7-yl)methyl)carbamate was prepared by reacting Intermediate 32 (13 mg, 0.03 mmol) and Intermediate 41 (12 mg, 0.04 mmol) under the same conditions described in the synthesis of Example 64 step 1. The reaction was purified by preparative HPLC (20-70% MeCN/H₂O+0.1% TFA) to obtain the title compound as a brown solid (8 mg, 39%). LCMS (ES⁺) Method 1A: m/z 628 (M+H)⁺, RT 2.20 min.

### Step 2: 6-(7-(aminomethyl)-7-(4-methylthiazol-2-yl)-3-azabicyclo[4.1.0]heptan-3-yl)-3-((2-(trifluoromethyl)pyridin-3-yl)thio)pyrazin-2-amine

6-(7-(Aminomethyl)-7-(4-methylthiazol-2-yl)-3-azabicyclo[4.1.0]heptan-3-yl)-3-((2-(trifluoromethyl)pyridin-3-yl)thio)pyrazin-2-amine was prepared from benzyl ((3-(6-amino-5-((2-(trifluoromethyl)pyridin-3-yl)thio)pyrazin-2-yl)-7-(4-methylthiazol-2-yl)-3-azabicyclo[4.1.0]heptan-7-yl)methyl)carbamate (8 mg, 0.013 mmol) according to the procedure described in the synthesis of Example 64 step 2. The reaction was purified by preparative HPLC (5-55% MeCN/H₂O+0.1% TFA) to afford the title compound as an off-white solid (TFA salt; 4 mg, 62%). ¹H NMR (300 MHz, DMSO-d₆+TFA) δ 8.48 (m, 1H), 7.95 (br s, 3H), 7.61 (s, 1H), 7.54 (m, 1H), 7.33 (d, J=8.2 Hz, 1H), 7.17 (br d, J=1.0 Hz, 1H), 4.34-4.16 (m, 2H), 3.64 (m, 1H), 3.49 (m, 1H), 3.27 (m, 1H), 2.80 (m, 1H), 2.36 (s, 3H), 2.28 (m, 1H), 2.00 (m, 1H), 1.82-1.61 (m, 2H); LCMS (ES⁺) Method 2: m/z 494 (M+H)⁺, RT 2.53 min.

### Example 74: 3-((2-amino-3-chloropyridin-4-yl)thio)-6-(7-(aminomethyl)-7-(4-methylthiazol-2-yl)-3-azabicyclo[4.1.0]heptan-3-yl)pyrazin-2-amine (Trifluoro Acetate Salt)

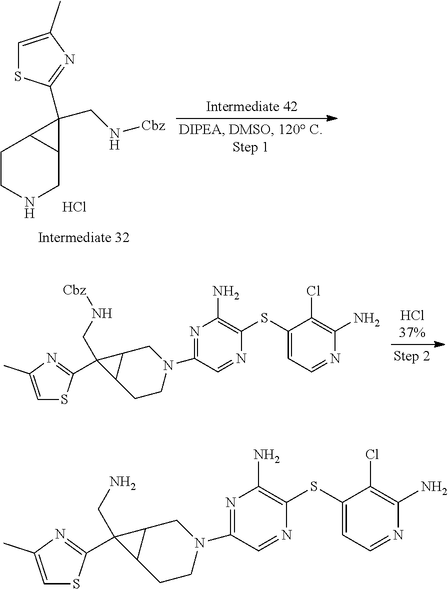 [SMILES: Cc1csc(C2(CNC(=O)OCc3ccccc3)C3CCNCC32)n1 ; Cl ; Cc1csc(C2(CNC(=O)OCc3ccccc3)C3CCN(c4cnc(Sc5ccnc(N)c5Cl)c(N)n4)CC32)n1 ; Cc1csc(C2(CN)C3CCN(c4cnc(Sc5ccnc(N)c5Cl)c(N)n4)CC32)n1]

### Step 1: benzyl ((3-(6-amino-5-((2-amino-3-chloropyridin-4-yl)thio)pyrazin-2-yl)-7-(4-methylthiazol-2-yl)-3-azabicyclo[4.1.0]heptan-7-yl)methyl)carbamate

Benzyl ((3-(6-amino-5-((2-amino-3-chloropyridin-4-yl)thio)pyrazin-2-yl)-7-(4-methylthiazol-2-yl)-3-azabicyclo[4.1.0]heptan-7-yl)methyl)carbamate was prepared by reacting Intermediate 32 (13 mg, 0.03 mmol) and Intermediate 42 (12 mg, 0.43 mmol), heating under MW irradiation. The reaction was purified by preparative HPLC (10-65% MeCN/H₂O+0.1% TFA) to obtain the title compound as a brown solid (5 mg, 25%). LCMS (ES⁺) Method 1A: m/z 609 (M+H)⁺, RT 1.61 min.

### Step 2: 3-((2-Amino-3-chloropyridin-4-yl)thio)-6-(7-(aminomethyl)-7-(4-methylthiazol-2-yl)-3-azabicyclo[4.1.0]heptan-3-yl)pyrazin-2-amine

The compound was prepared from benzyl ((3-(6-amino-5-((2-amino-3-chloropyridin-4-yl)thio)pyrazin-2-yl)-7-(4-methylthiazol-2-yl)-3-azabicyclo[4.1.0]heptan-7-yl)methyl)carbamate (5 mg, 0.008 mmol) according to the procedure described in the synthesis of Example 64 step 2. The reaction was purified by preparative HPLC (0-35% MeCN/H₂O+0.1% TFA) to afford the title compound as an off-white solid (TFA salt; 2 mg, 49%). ¹H NMR (300 MHz, DMSO-d₆+TFA) δ 8.30 (s, 1H) 7.96 (br s, 3H), 7.75 (d, J=6.9 Hz, 1H), 7.64 (s, 1H), 7.17 (br d, J=1.0 Hz, 1H), 6.09 (d, J=6.9 Hz, 1H), 4.33-4.15 (m, 2H), 3.68 (m, 1H), 3.51 (m, 1H), 3.23 (m, 1H), 2.83 (m, 1H), 2.36 (s, 3H), 2.29 (m, 1H), 2.00 (m, 1H), 1.81-1.65 (m, 2H); LCMS (ES⁺) m/z Method 2: 475 (M+H)⁺, RT 1.78 min.

### Example 75: 6-(7-(aminomethyl)-3-azabicyclo[4.1.0]heptan-3-yl)-3-((2-(trifluoromethyl)pyridin-3-yl)thio)pyrazin-2-amine (Trifluoro Acetate Salt)

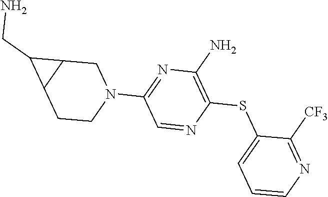 [SMILES: NCC1C2CCN(c3cnc(Sc4cccnc4C(F)(F)F)c(N)n3)CC12]

### Step 1: 2-((3-(6-amino-5-((2-(trifluoromethyl)pyridin-3-yl)thio)pyrazin-2-yl)-3-azabicyclo[4.1.0]heptan-7-yl)methyl)isoindoline-1,3-dione

2-((3-(6-Amino-5-((2-(trifluoromethyl)pyridin-3-yl)thio)pyrazin-2-yl)-3-azabicyclo[4.1.0]heptan-7-yl)methyl)isoindoline-1,3-dione was prepared by reacting Intermediate 38 (20 mg, 0.07 mmol) and Intermediate 41 (20 mg, 0.08 mmol) under the same conditions described in the synthesis of Example 64 step 1. The reaction was purified by preparative HPLC (20-75% MeCN/H₂O+0.1% TFA) to afford the title compound as yellow solid (20 mg, 55%). LCMS (ES⁺) Method 1A: m/z 527 (M+H)⁺, RT 2.28 min.

### Step 2: 6-(7-(aminomethyl)-3-azabicyclo[4.1.0]heptan-3-yl)-3-((2-(trifluoromethyl)pyridin-3-yl)thio)pyrazin-2-amine

To a solution of 2-((3-(6-amino-5-((2-(trifluoromethyl)pyridin-3-yl)thio)pyrazin-2-yl)-3-azabicyclo[4.1.0]heptan-7-yl)methyl)isoindoline-1,3-dione (20 mg, 0.04 mmol) in EtOH (2 mL) N₂H₄·H₂O (27 uL, 0.54 mmol) was added and the resulting mixture was stirred at rt for 3 h. The reaction was purified by preparative HPLC (5-20% MeCN/H₂O+0.1% TFA) to obtain the title compound as an off-white solid (TFA salt; 12 mg, 81%). ¹H NMR (300 MHz, DMSO-d₆+TFA) δ 8.47-8.43 (m, 1H), 7.79-7.63 (m, 3H), 7.56-7.49 (m, 2H), 7.32 (d, J=7.1 Hz, 1H), 4.02 (d, J=13.3 Hz, 1H), 3.76-3.66 (m, 1H), 3.62-3.50 (m, 1H), 3.29-3.16 (m, 1H), 2.83-2.65 (m, 2H), 2.09-1.96 (m, 1H), 1.84-1.70 (m, 1H), 1.20-1.11 (m, 2H), 0.91-0.79 (m, 1H); LCMS (ES⁺) Method 2: m/z 397 (M+H)⁺, RT 2.20 min.

### Example 76: 4-((5-(7-(aminomethyl)-7-(4-methylthiazol-2-yl)-3-azabicyclo[4.1.0]heptan-3-yl)imidazo[1,2-c]pyrimidin-8-yl)thio)-3-chloropyridin-2-amine (Trifluoro Acetate Salt)

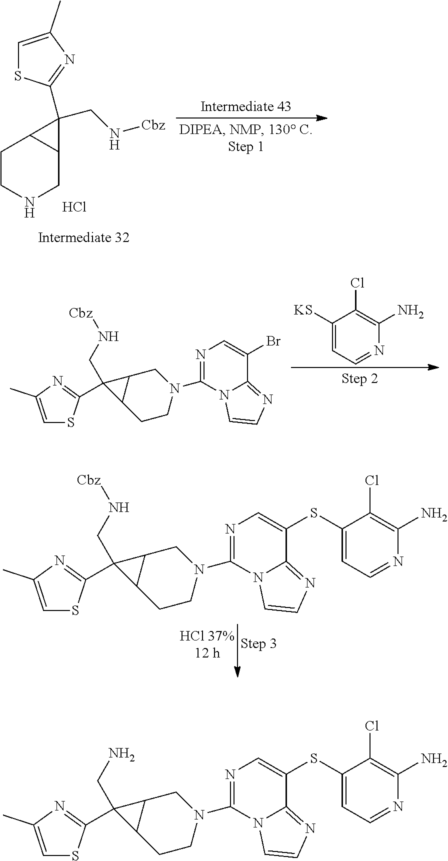

### Step 2: benzyl ((3-(8-((2-amino-3-chloropyridin-4-yl)thio)imidazo[1,2-c]pyrimidin-5-yl)-7-(4-methylthiazol-2-yl)-3-azabicyclo[4.1.0]heptan-7-yl)methyl)carbamate

To a mixture of benzyl ((3-(8-bromoimidazo[1,2-c]pyrimidin-5-yl)-7-(4-methylthiazol-2-yl)-3-azabicyclo[4.1.0]heptan-7-yl)methyl)carbamate (19 mg, 0.03 mmol), potassium 2-amino-3-chloropyridine-4-thiolate (prepared with the same procedure used for Intermediate 41 step 2; 14 mg, 0.07 mmol), XantPhos (6 mg, 0.01 mmol) and Pd₂(dba)₃ (9.4 mg, 0.01 mmol) in degassed dry 1,4-dioxane (0.5 mL), dry DIPEA (0.03 mL, 0.17 mmol) was added and the reaction mixture was stirred at 130° C. for 3.5 h. The reaction was diluted with EtOAc and H₂O, then the organic layer was washed with 5% citric acid solution, dried over Na₂SO₄, filtered and concentrated in vacuo. The resulting crude was purified by preparative HPLC (5-55% MeCN/H₂O+0.1% TFA) to obtain the title compound as a white solid (TFA salt; 14 mg, 64%). LCMS (ES⁺) Method 1A: m/z 633 (M+H)⁺, RT 1.54 min.

### Step 3: 4-((5-(7-(aminomethyl)-7-(4-methylthiazol-2-yl)-3-azabicyclo[4.1.0]heptan-3-yl)imidazo[1,2-c]pyrimidin-8-yl)thio)-3-chloropyridin-2-amine

4-((5-(7-(aminomethyl)-7-(4-methylthiazol-2-yl)-3-azabicyclo[4.1.0]heptan-3-yl)imidazo[1,2-c]pyrimidin-8-yl)thio)-3-chloropyridin-2-amine was prepared from benzyl ((3-(8-((2-amino-3-chloropyridin-4-yl)thio)imidazo[1,2-c]pyrimidin-5-yl)-7-(4-methylthiazol-2-yl)-3-azabicyclo[4.1.0]heptan-7-yl)methyl)carbamate (14 mg, 0.02 mmol) according to the procedure described in the synthesis of Example 64 step 2. The reaction was purified by preparative HPLC (0-35% MeCN/H₂O+0.1% TFA) to obtain the title compound as a white solid (TFA salt; 2 mg, 15%). ¹H NMR (300 MHz, DMSO-d₆+TFA) δ 8.42 (s, 1H), 8.13-7.98 (m, 5H), 7.73 (d, J=6.8 Hz, 1H), 7.20 (br d, J=1.0 Hz, 1H), 6.37 (d, J=6.8 Hz, 1H), 4.13-3.98 (m, 2H), 3.92 (m, 1H), 3.64 (m, 2H), 3.08 (m, 1H), 2.43 (m, 1H), 2.37 (s, 3H), 2.19 (m, 1H), 2.05 (m, 1H), 1.92 (m, 1H); LCMS (ES⁺) Method 2: m/z 499, 501 (M+H)⁺, RT 1.69 min.

### Example 77: (3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

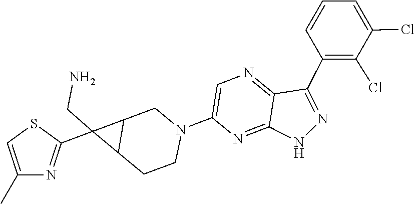 [SMILES: Cc1csc(C2(CN)C3CCN(c4cnc5c(-c6cccc(Cl)c6Cl)n[nH]c5n4)CC32)n1]

### Step 1: benzyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

A mixture of Intermediate 44 (70 mg, 0.19 mmol), Intermediate 32 (72 mg, 0.18 mmol) and dry DIPEA (0.19 mL, 1.09 mmol) in dry DMF (1.7 mL) was heated to 80° C. in a sealed vial for 16 h. The reaction was diluted with EtOAc and washed with 5% citric acid solution (×2), H₂O (×2) and brine (×2). The organic layer was dried over Na₂SO₄, filtered and concentrated in vacuo to obtain a crude compound which was used in the next step without further purification (124 mg). LCMS (ES⁺) Method 1A: m/z 686 (M+H)⁺, RT 2.32 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

A mixture of benzyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (50 mg, 0.07 mmol), K₃PO₄ (94 mg, 0.44 mmol), 2,3-dichlorophenylboronic acid (28 mg, 0.15 mmol) and Pd(dppf)Cl₂·DCM (12 mg, 0.010 mmol) in degassed 1,4-dioxane/H₂O (9:1; 2.5 mL) was stirred for 1 h at 80° C. The black suspension was diluted with EtOAc and washed with brine (×3). Organic layer was dried over Na₂SO₄, filtered and concentrated in vacuo. The resulting crude was purified by flash chromatography (30-70% EtOAc in petroleum ether) to afford the title compound as a yellow oil (44 mg, 86% over two steps). LCMS (ES⁺) Method 1A: m/z 704, 706 (M+H)⁺, RT 2.62 min.

### Step 3: (3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine

Benzyl ((3-(3-(2,3-dichlorophenyl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (44 mg, 0.06 mmol) was dissolved in conc. HCl (37%; 2 mL) and the resulting yellow solution was stirred at rt for 12 h. The reaction was directly purified by preparative HPLC (5-55% MeCN/H₂O+0.1% TFA) to obtain the title compound as a light-yellow solid (TFA salt; 16 mg, 43%). ¹H NMR (300 MHz, DMSO-d₆+TFA) δ 8.40 (s, 1H), 7.92 (br s, 3H), 7.72 (m, 2H), 7.49 (t, J=7.9 Hz, 1H), 7.17 (br d, J=1.0 Hz, 1H), 4.43-4.26 (m, 2H), 3.73 (m, 1H), 3.52 (m, 1H), 3.31 (m, 1H), 2.96 (m, 1H), 2.36 (m, 4H), 2.03 (m, 1H), 1.92-1.74 (m, 2H); LCMS (ES⁺) Method 2: m/z 486,488 (M+H)⁺, RT 2.67 min.

The following examples were synthesized using the above procedure with Intermediates 32, 44 and the appropriate commercial boronic acid.

| Example # | I# | Boronic ester or acid | ¹H NMR (300 MHz) |
|---|---|---|---|
| 84 | 0422 | 2-chlorophenylboronic | (DMSO-d₆ + TFA) δ 8.39 (s, 1H), 7.92 (br s, 3H), 7.79- |
| | 539 | acid | 7.71 (m, 1H), 7.64-7.56 (m, 1H), 7.53-7.42 (m, 2H), |
| | | | 7.17 (br d, J = 1.0 Hz, 1H), 4.45-4.26 (m, 2H), 3.74 (m, |
| | | | 1H), 3.52 (m, 1H), 3.32 (m, 1H), 2.96 (m, 1H), 2.42- |
| | | | 2.29 (m, 4H), 2.03 (m, 1H), 1.93-1.71 (m, 2H) |
| 85 | 0422 | 3-chlorophenylboronic | (DMSO-d₆ + TFA) δ 8.48 (s, 1H), 8.41 (m, 1H), 8.29 |
| | 596 | acid | (m, 1H), 7.92 (br s, 3H), 7.53 (t, J = 7.9 Hz, 1H), 7.44 |
| | | | (m, 1H), 7.17 (br d, J = 1.0 Hz, 1H), 4.45-4.26 (m, 2H), |
| | | | 3.77 (m, 1H), 3.50 (m, 1H), 3.29 (m, 1H), 2.98 (m, |
| | | | 1H), 2.42-2.30 (m, 4H), 2.03 (m, 1H), 1.90-1.74 (m, |
| | | | 2H) |
| 86 | 0422 | 2,3- | (DMSO-d₆ + TFA) δ 8.45 (s, 1H), 8.01 (m, 1H), 7.92 |
| | 538 | difluorophenylboronic | (br s, 3H), 7.49 (m, 1H), 7.36 (m, 1H), 7.18 (br d, |
| | | acid | J = 1.0 Hz, 1H), 4.46-4.26 (m, 2H), 3.76 (m, 1H), 3.51 |
| | | | (m, 1H), 3.30 (m, 1H), 2.98 (m, 1H), 2.42-2.30 (m, |
| | | | 4H), 2.03 (m, 1H), 1.90-1.74 (m, 2H) |
| 87 | 0422 | 2-chloro-3- | (DMSO-d₆ + TFA) δ 8.38 (s, 1H), 7.92 (br s, 3H), 7.42 |
| | 542 | methoxyphenylboronic | (t, J = 7.9 Hz, 1H), 7.29-7.21 (m, 2H), 7.17 (br d, J = 1.0 |
| | | acid | Hz, 1H), 4.43-4.26 (m, 2H), 3.92 (s, 3H), 3.72 (m, 1H), |
| | | | 3.52 (m, 1H), 3.31 (m, 1H), 2.95 (m, 1H), 2.42-2.32 |
| | | | (m, 4H), 2.02 (m, 1H), 1.90-1.74 (m, 2H) |
| 88 | 0422 | quinoline-5-boronic | (DMSO-d₆ + TFA) δ 10.10 (d, J = 8.7 Hz, 1H), 9.41 (dd, |
| | 752 | acid | J = 5.3, 1.4 Hz, 1H), 8.79 (dd, J = 6.4, 2.1 Hz, 1H), 8.51 |
| | | | (s, 1H), 8.38-8.26 (m, 2H), 8.19 (dd, J = 8.7, 5.3 Hz, |
| | | | 1H), 7.95 (br s, 3H), 7.18 (br d, J = 1.0 Hz, 1H), 4.44- |
| | | | 4.28 (m, 2H), 3.82 (m, 1H), 3.54 (m, 1H), 3.31 (m, |
| | | | 1H), 3.03 (m, 1H), 2.44-2.31 (m, 4H), 2.05 (m, 1H), |
| | | | 1.92-1.76 (m, 2H) |
| 89 | 0423 | naphthalen-1- | (DMSO-d₆ + TFA) δ 8.69 (m, 1H), 8.43 (s, 1H), 8.16 |
| | 593 | ylboronic acid | (dd, J = 7.2, 1.1 Hz, 1H), 8.06-7.98 (m, 2H), 7.94 (br s, |
| | | | 3H), 7.65 (m, 1H), 7.61-7.50 (m, 2H), 7.18 (brd, J = 1.0 |
| | | | Hz, 1H), 4.48-4.28 (m, 2H), 3.77 (dd, J = 14.6, 6.2 Hz, |
| | | | 1H), 3.53 (m, 1H), 3.33 (m, 1H), 2.99 (m, 1H), 2.43- |
| | | | 2.29 (m, 4H), 2.04 (dt, J = 9.8, 3.7 Hz, 1H), 1.94-1.74 |
| | | | (m, 2H) |
| 90 | 0422 | (2- | (DMSO-d₆) δ 13.56 (s, 1H), 8.86 (br d, J = 4.7 Hz, 1H), |
| | 202 | (trifluoromethyl)pyridin- | 8.41 (s, 1H), 8.27 (br d, J = 8.1 Hz, 1H), 8.00-7.83 (m, |
| | | 3-yl)boronic acid | 4H), 7.19 (br d, J = 1.0 Hz, 1H), 4.41-4.27 (m, 2H), |
| | | | 3.76 (m, 1H), 3.51 (m, 1H), 3.30 (m, 1H), 2.98 (m, |
| | | | 1H), 2.42-2.30 (m, 4H), 2.03 (m, 1H), 1.91-1.74 (m, |
| | | | 2H) |

### Example 78: ((1S,6R,7S)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

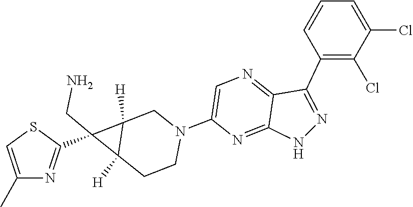 [SMILES: Cc1csc([C@@]2(CN)[C@@H]3CCN(c4cnc5c(-c6cccc(Cl)c6Cl)n[nH]c5n4)C[C@@H]32)n1]

### Step 1: benzyl (((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

The compound was prepared according to the procedure described in the Example 77 step 1 using Intermediate 39 (21 mg, 0.05 mmol) instead of Intermediate 32. LCMS (ES⁺) Method 1A: m/z 686 (M+H)⁺, RT 2.32 min.

### Step 2: benzyl (((1S,6R,7S)-3-(3-(2,3-dichlorophenyl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

Benzyl (((1S,6R,7S)-3-(3-(2,3-dichlorophenyl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate was prepared from benzyl (((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (15 mg, 0.022 mmol) using the same procedure described in the synthesis of Example 77 step 2. LCMS (ES⁺) Method 1A: m/z 704, 706 (M+H)⁺, RT 2.62 min.

### Step 3: ((1S,6R,7S)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine

((1S,6R,7S)-3-(3-(2,3-Dichlorophenyl)-1H-pyrazolo[3,4-b]pyrazin-6-yl)-7-(4-methylthiazol-2-yl)-3-azabicyclo[4.1.0]heptan-7-yl)methanamine was prepared from benzyl (((1S,6R,7S)-3-(3-(2,3-dichlorophenyl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (15 mg, 0.02 mmol) using the same procedure described in the synthesis of Example 77 step 3. The reaction was directly purified by preparative HPLC (5-55% MeCN/H₂O+0.1% TFA) to obtain the title compound as a light-yellow solid (TFA salt; 16 mg, 43%). ¹H NMR (300 MHz, DMSO-d₆+TFA) δ 8.40 (s, 1H), 7.92 (br s, 3H), 7.72 (m, 2H), 7.49 (t, J=7.9 Hz, 1H), 7.17 (br d, J=1.0 Hz, 1H), 4.43-4.26 (m, 2H), 3.73 (m, 1H), 3.52 (m, 1H), 3.31 (m, 1H), 2.96 (m, 1H), 2.36 (m, 4H), 2.03 (m, 1H), 1.92-1.74 (m, 2H); LCMS (ES⁺) Method 2: m/z 486, 488 (M+H)⁺, RT 2.67 min.

### Example 79: ((1S,6R,7S)-3-(3-(4-chloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

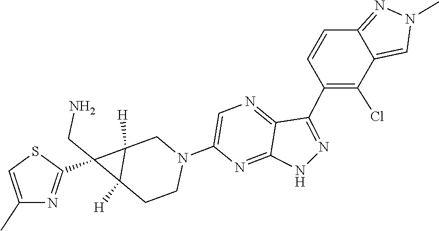

### Step 1: benzyl (((1S,6R,7S)-3-(3-(2,4-dimethyl-2H-indazol-5-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

Benzyl (((1S,6R,7S)-3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in the synthesis of Example 77 step 1; 20 mg, 0.03 mmol) and Intermediate 45 (17 mg, 0.06 mmol) were reacted using the same procedure described in the synthesis of Example 77 step 2. The resulting crude was purified by flash chromatography (0-100% EtOAc in petroleum ether) to afford the title compound as a yellow oil (6.6 mg, 31%). LCMS (ES⁺) Method 1A: m/z 724, 726 (M+H)⁺, RT 2.30 min.

### Step 2: ((1S,6R,7S)-3-(3-(4-chloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine

((1S,6R,7S)-3-(3-(4-chloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine was prepared from benzyl (((1S,6R,7S)-3-(3-(4-chloro-2-methyl-2H-indazol-5-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (6.5 mg, 0.009 mmol) using the same procedure described in the synthesis of Example 77 step 3. The reaction was directly purified by preparative HPLC (0-45% MeCN/H₂O+0.1% TFA) to obtain the title compound as a light-yellow solid (TFA salt; 3 mg, 48%). ¹H NMR (300 MHz, DMSO-d₆+TFA) δ 8.52 (s, 1H) 8.40 (s, 1H), 7.98-7.86 (m, 3H), 7.70-7.61 (m, 2H), 7.18-7.12 (m, 1H), 4.46-4.29 (m, 2H), 4.23 (s, 3H), 3.77-3.68 (m, 1H), 3.56-3.47 (m, 1H), 3.38-3.28 (m, 1H), 3.02-2.89 (m, 1H), 2.35 (s, 4H), 2.08-1.97 (m, 1H), 1.90-1.78 (m, 2H). LCMS (ES⁺) Method 2: m/z 506, 508 (M+H)⁺, RT 2.24 min.

### Example 80: (3-(3-(2,3-dichlorophenyl)-1H-pyrazolo[3,4-b]pyrazin-6-yl)-7-phenyl-3-azabicyclo[4.1.0]heptan-7-yl)methanamine (Trifluoro Acetate Salt)

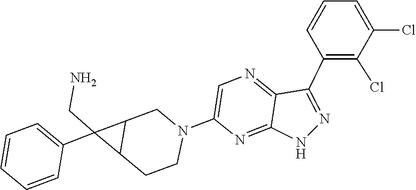 [SMILES: NCC1(c2ccccc2)C2CCN(c3cnc4c(-c5cccc(Cl)c5Cl)n[nH]c4n3)CC21]

### Step 1: benzyl ((3-(3-iodo-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate was prepared by reacting Intermediate 28 (35 mg, 0.094 mmol) and Intermediate 44 (70 mg, 0.19 mmol) according to the procedure described in the synthesis of Example 77 step 1. The crude compound was used in the next step without further purification (60 mg). LCMS (ES⁺) Method 1A: m/z 665 (M+H)⁺, RT 2.61 min.

### Step 2: benzyl ((3-(3-(2,3-dichlorophenyl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate

Benzyl ((3-(3-(2,3-dichlorophenyl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate was prepared according to the procedure described in the synthesis of Example 77 step 2, starting from benzyl ((3-(3-iodo-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate (25 mg, 0.04 mmol). The residue was purified by flash chromatography (0-30% EtOAc in petroleum ether) to give the title compound as an off-white solid (16 mg, 62% over two steps). LCMS (ES⁺) Method 1A: m/z 684, 686 (M+H)⁺, RT 2.39 min.

### Step 3: (3-(3-(2,3-dichlorophenyl)-1H-pyrazolo[3,4-b]pyrazin-6-yl)-7-phenyl-3-azabicyclo [4.1.0]heptan-7-yl)methanamine

(3-(3-(2,3-Dichlorophenyl)-1H-pyrazolo[3,4-b]pyrazin-6-yl)-7-phenyl-3-azabicyclo [4.1.0]heptan-7-yl)methanamine was prepared according to the procedure described in the synthesis of Example 77 step 3 starting from benzyl ((3-(3-(2,3-dichlorophenyl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate (16 mg, 0.023 mmol). The reaction was directly purified by preparative HPLC (10-60% MeCN/H₂O+0.1% TFA) to obtain the title compound as a light-yellow solid (TFA salt; 4 mg, 37%). ¹H NMR (300 MHz, DMSO-d₆+TFA) δ 8.41 (s, 1H), 7.72 (m, 2H), 7.63 (br s, 3H), 7.54-7.44 (m, 3H), 7.38 (m, 2H), 7.29 (m, 1H), 4.50-4.24 (m, 2H), 3.72 (m, 1H), 3.26 (m, 1H), 3.15 (m, 1H), 2.92 (m, 1H), 2.33 (m, 1H), 1.89-1.67 (m, 2H), 1.53 (m, 1H); LCMS (ES⁺) Method 2: m/z 465, 467 (M+H)⁺, RT 2.75 min.

### Example 81: (3-(3-(4-chloro-2-methyl-2H-indazol-5-yl)-1H-pyrazolo[3,4-b]pyrazin-6-yl)-7-phenyl-3-azabicyclo[4.1.0]heptan-7-yl)methanamine (Trifluoro Acetate Salt)

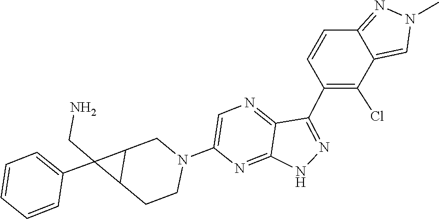 [SMILES: Cn1cc2c(Cl)c(-c3n[nH]c4nc(N5CCC6C(C5)C6(CN)c5ccccc5)cnc34)ccc2n1]

### Step 1: benzyl ((3-(3-(4-chloro-2-methyl-2H-indazol-5-yl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate

Benzyl ((3-(3-(4-chloro-2-methyl-2H-indazol-5-yl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate was prepared from benzyl ((3-(3-iodo-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate (15 mg, 0.023 mmol) and Intermediate 45 (13 mg, 0.04 mmol) according to the procedure described in the synthesis of Example 77 step 2. The residue was purified by flash chromatography (0-100% EtOAc in petroleum ether) to give the title compound as an off-white solid (5 mg, 31% over two steps). LCMS (ES⁺) Method 1A: m/z 703, 705 (M+H)⁺, RT 2.58 min.

### Step 2: (3-(3-(4-chloro-2-methyl-2H-indazol-5-yl)-1H-pyrazolo[3,4-b]pyrazin-6-yl)-7-phenyl-3-azabicyclo[4.1.0]heptan-7-yl)methanamine

(3-(3-(4-Chloro-2-methyl-2H-indazol-5-yl)-1H-pyrazolo[3,4-b]pyrazin-6-yl)-7-phenyl-3-azabicyclo[4.1.0]heptan-7-yl)methanamine was prepared from benzyl ((3-(3-(4-chloro-2-methyl-2H-indazol-5-yl)-1-(tetrahydro-2H-pyran-2-yl)-1H-pyrazolo[3,4-b]pyrazin-6-yl)-7-phenyl-3-azabicyclo[4.1.0]heptan-7-yl)methyl)carbamate (4.5 mg, 0.006 mmol) according to the procedure described in the synthesis of Example 77 step 3. The reaction was directly purified by preparative HPLC (5-50% MeCN/H₂O+0.1% TFA) to obtain the title compound as a light-yellow solid (TFA salt; 2 mg, 62%). ¹H NMR (300 MHz, DMSO-d₆+TFA) δ 8.52 (s, 1H), 8.40 (s, 1H), 7.66 (m, 5H), 7.50-7.45 (m, 2H), 7.36 (t, J=7.7 Hz, 2H), 7.31-7.23 (m, 1H), 4.45 (d, J=13.7 Hz, 1H), 4.38-4.30 (m, 1H), 4.23 (s, 3H), 3.71 (dd, J=14.3, 6.4 Hz, 1H), 3.35-3.12 (m, 2H), 2.98-2.85 (m, 1H), 2.40-2.31 (m, 1H), 1.86-1.75 (m, 2H), 1.56-1.50 (m, 1H); LCMS (ES⁺) Method 2A: m/z 485, 487 (M+H)⁺, RT 2.32 min.

### Example 82: (3-(3-(2,3-dichlorophenyl)-1H-pyrazolo[3,4-b]pyrazin-6-yl)-7-(pyridin-2-yl)-3-azabicyclo [4.1.0]heptan-7-yl)methanamine (Trifluoro Acetate Salt)

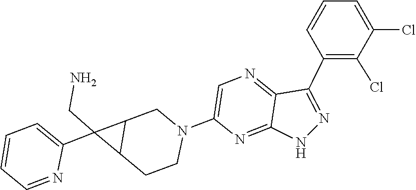 [SMILES: NCC1(c2ccccn2)C2CCN(c3cnc4c(-c5cccc(Cl)c5Cl)n[nH]c4n3)CC21]

### Step 1: benzyl ((3-(3-iodo-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate was prepared by reacting Intermediate 29 (18 mg, 0.044 mmol) and Intermediate 44 (66 mg, 0.09 mmol) according to the procedure described in the synthesis of Example 77 step 1. The crude title compound was used in the next step without further purification (29 mg). LCMS (ES⁺) Method 1A: m/z 666 (M+H)⁺, RT 1.71 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate

Benzyl ((3-(3-(2,3-dichlorophenyl)-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate was prepared according to the procedure described in the synthesis of Example 77 step 2, starting from benzyl ((3-(3-iodo-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate (29 mg, 0.044 mmol). The residue was purified by flash chromatography (30-80% EtOAc in petroleum ether) to give the title compound as an off-white foam (15 mg, 50% over two steps). Method 1A m/z 684, 686 (M+H)⁺, RT 2.01 min.

### Step 3: (3-(3-(2,3-dichlorophenyl)-1H-pyrazolo[3,4-b]pyrazin-6-yl)-7-(pyridin-2-yl)-3-azabicyclo [4.1.0]heptan-7-yl)methanamine

The compound was prepared from benzyl ((3-(3-(2,3-dichlorophenyl)-1-(tetrahydro-2H-pyran-2-yl)-1H-pyrazolo[3,4-b]pyrazin-6-yl)-7-(pyridin-2-yl)-3-azabicyclo[4.1.0]heptan-7-yl)methyl)carbamate (15 mg, 0.02 mmol) according to the procedure described in the synthesis of Example 77 step 3. The reaction was directly purified by preparative HPLC (0-40% MeCN/H₂O+0.1% TFA) to obtain the title compound as a light-yellow solid (TFA salt; 8 mg, 63%). ¹H NMR (300 MHz, DMSO-d₆+TFA) δ 8.76 (dd, J=5.6, 1.5 Hz, 1H), 8.41 (s, 1H), 8.22 (dt, J=7.8, 1.5 Hz, 1H), 7.84 (br s, 3H), 7.78-7.62 (m, 4H), 7.50 (t, J=7.9 Hz, 1H), 4.53-4.30 (m, 2H), 3.70 (m, 1H), 3.47 (m, 1H), 3.29 (m, 1H), 2.96 (m, 1H), 2.34 (m, 1H), 2.12 (m, 1H), 1.94-1.75 (m, 2H); LCMS (ES⁺) Method 2: m/z 466, 468 (M+H)⁺, RT 2.29 min.

### Example 83: (3-(3-(2,3-dichlorophenyl)-1H-pyrazolo[3,4-b]pyrazin-6-yl)-7-(pyrimidin-2-yl)-3-azabicyclo [4.1.0]heptan-7-yl)methanamine (Trifluoro Acetate Salt)

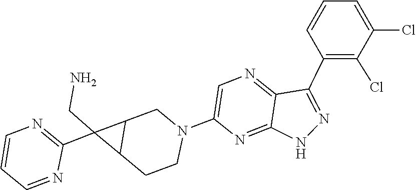 [SMILES: NCC1(c2ncccn2)C2CCN(c3cnc4c(-c5cccc(Cl)c5Cl)n[nH]c4n3)CC21]

### Step 1: benzyl ((3-(3-iodo-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate was prepared by reacting Intermediate 30 (8.5 mg, 0.023 mmol) and Intermediate 44 (34 mg, 0.05 mmol) according to the procedure described in the synthesis of Example 77 step 1. The crude title compound was used in the next step without further purification (15 mg). LCMS (ES⁺) Method 1A: m/z 667 (M+H)⁺, RT 2.43 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate

Benzyl ((3-(3-(2,3-dichlorophenyl)-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate was prepared according to the procedure described in the synthesis of Example 77 step 2, starting from benzyl ((3-(3-iodo-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate (15 mg, 0.023 mmol). The crude title compound was used in the next step without further purification (15 mg). LCMS (ES⁺) Method 1A: m/z 685, 687 (M+H)⁺, RT 2.74 min.

### Step 3: (3-(3-(2,3-dichlorophenyl)-1H-pyrazolo[3,4-b]pyrazin-6-yl)-7-(pyrimidin-2-yl)-3-azabicyclo [4.1.0]heptan-7-yl)methanamine

Benzyl ((3-(3-(2,3-dichlorophenyl)-1-(tetrahydro-2H-pyran-2-yl)-1H-pyrazolo[3,4-b]pyrazin-6-yl)-7-(pyrimidin-2-yl)-3-azabicyclo[4.1.0]heptan-7-yl)methyl)carbamate (15 mg, 0.02 theoretical mmol) was treated with HCl (4N in dioxane; 0.5 mL, 2.0 mmol) and the resulting solution was stirred at rt for 30 min. The reaction mixture was directly purified by preparative HPLC (30-90% MeCN/H₂O+0.1% TFA) to obtain the product (without THP protecting group) as a yellow solid (8 mg). The compound was then dissolved in conc. HCl (37%; 0.5 mL) and stirred at rt for 18 h. The reaction was directly purified by preparative HPLC (10-55% MeCN/H₂O+0.1% TFA) to afford the title compound as an off-white powder (TFA salt; 0.7 mg, 5% over three steps). LCMS (ES⁺) Method 2: m/z 467, 469 (M+H)⁺, RT 2.50 min.

### Example 91: (3-(3-(Benzo[d][1,3]dioxol-4-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

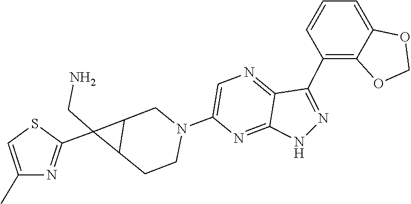 [SMILES: Cc1csc(C2(CN)C3CCN(c4cnc5c(-c6cccc7c6OCO7)n[nH]c5n4)CC32)n1]

### Step 1: 2-((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)isoindoline-1,3-dione

Intermediate 36 (107 mg, 0.273 mmol) was reacted with Intermediate 44 (104 mg, 0.28 mmol) according to the procedure described in the synthesis of Example 77 step 1. The crude title compound was used in the next step without further purification (215 mg). LCMS (ES⁺) Method 1A: m/z 682 (M+H)⁺, RT 2.28 min.

### Step 2: tert-butyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

2-((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)isoindoline-1,3-dione (186 mg, 0.27 mmol) was suspended in EtOH (8 mL), N₂H₄·H₂O (0.41 mL, 8.19 mmol) was added and the reaction mixture was stirred at rt for 3 h. The volatiles were removed under reduced pressure to obtain crude primary amine (231 mg). The residue was taken up in DCM (5 mL) then a solution of (Boc)₂O (119 mg, 0.55 mmol) in DCM (1 mL) and DIPEA (0.19 mL, 1.09 mmol) were added, and the reaction mixture was stirred at rt for 1.5 h. The reaction was diluted with DCM and washed with 5% citric acid solution (×2). The aqueous phase was extracted again with DCM (×2) then the collected organic layers were dried over Na₂SO₄, filtered and concentrated under reduced pressure. The resulting crude was purified by flash chromatography (30-50% EtOAc in petroleum ether) to afford the title compound as a yellow foam (161 mg, 90% over two steps). ¹H NMR (300 MHz, CDCl₃) δ 8.18 (d, J=2.9 Hz, 1H), 6.78 (s, 1H), 5.80 (dd, J=10.4, 2.2 Hz, 1H), 5.43 (br s, 1H), 4.38-4.07 (m, 3H), 3.98 (m, 1H), 3.89-3.63 (m, 3H), 3.15 (m, 1H), 2.68 (m, 1H), 2.47 (s, 3H), 2.39 (m, 1H), 2.17 (m, 1H), 2.10-1.87 (m, 4H), 1.79 (m, 2H), 1.58 (m, 1H), 1.41 (s, 9H); LCMS (ES⁺) Method 1A: m/z 652 (M+H)⁺, RT 2.25 min.

### Step 3: tert-butyl ((3-(3-(benzo[d][1,3]dioxol-4-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

Tert-butyl((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (10 mg, 0.015 mmol) and benzo[d][1,3]dioxol-4-ylboronic acid (5 mg, 0.031 mmol) were reacted according to the procedure described in the synthesis of Example 77 step 2. The residue was purified by flash chromatography (30-70% EtOAc in petroleum ether) to give the title compound as a yellow oil (6 mg, 61%). LCMS (ES⁺) Method 1A: m/z 646 (M+H)⁺, RT 2.36 min.

### Step 4: 3-(3-(benzo[d][1,3]dioxol-4-yl)-1H-pyrazolo[3,4-b]pyrazin-6-yl)-7-(4-methylthiazol-2-yl)-3-azabicyclo[4.1.0]heptan-7-yl)methanamine

Tert-butyl ((3-(3-(benzo[d][1,3]dioxol-4-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (6 mg, 0.009 mmol) was dissolved in DCM (0.5 mL) and treated with HCl (4N in dioxane; 0.2 mL, 0.8 mmol). The solution was stirred at rt for 1 h, then solvent was removed in vacuo. The resulting crude was purified by preparative HPLC (5-50% MeCN/H₂O+0.1% TFA) to afford the title compound as an off-white solid (TFA salt; 4 mg, 77%). ¹H NMR (300 MHz, DMSO-d₆+TFA) δ 8.43 (s, 1H), 8.04-7.81 (m, 4H), 7.17 (br d, J=1.0 Hz, 1H), 7.02-6.91 (m, 2H), 6.10 (s, 2H), 4.48-4.26 (m, 2H), 3.75 (dd, J=14.6, 6.3 Hz, 1H), 3.51 (m, 1H), 3.30 (m, 1H), 3.06-2.87 (m, 1H), 2.43-2.30 (m, 4H), 2.03 (m, 1H), 1.91-1.73 (m, 2H); LCMS (ES⁺) Method 2A: m/z 462 (M+H)⁺, RT 2.40 min.

### Example 92: (3-(3-(8-fluoroquinolin-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

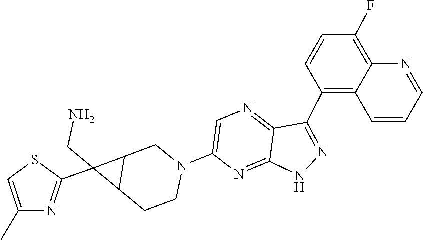 [SMILES: Cc1csc(C2(CN)C3CCN(c4cnc5c(-c6ccc(F)c7ncccc67)n[nH]c5n4)CC32)n1]

### Step 1: tert-butyl ((3-(3-(8-fluoroquinolin-5-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

Tert-butyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in the synthesis of Example 91 step 2; 16 mg, 0.025 mmol) and 8-fluoro-5-(4,4,5,5-tetramethyl-1,3,2-dioxaborolan-2-yl)quinoline (13.4 mg, 0.049 mmol) were reacted according to the procedure described in the synthesis of Example 77 step 2. The residue was purified by flash chromatography (50-100% EtOAc in petroleum ether) to give the title compound as a yellow oil (16 mg, 97%). LCMS (ES⁺) Method 1A: m/z 671 (M+H)⁺, RT 2.22 min.

### Step 2: (3-(3-(8-fluoroquinolin-5-yl)-1H-pyrazolo[3,4-b]pyrazin-6-yl)-7-(4-methylthiazol-2-yl)-3-azabicyclo[4.1.0]heptan-7-yl)methanamine

(3-(3-(8-Fluoroquinolin-5-yl)-1H-pyrazolo[3,4-b]pyrazin-6-yl)-7-(4-methylthiazol-2-yl)-3-azabicyclo[4.1.0]heptan-7-yl)methanamine was prepared from tert-butyl ((3-(3-(8-fluoroquinolin-5-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (16 mg, 0.024 mmol) according to the procedure described in the synthesis of Example 91 step 4. The residue was directly purified by preparative HPLC (5-45% MeCN/H₂O+0.1% TFA) to obtain the title compound as a yellow solid (TFA salt; 13 mg, 92%). ¹H NMR (300 MHz, DMSO-d₆+TFA) δ 9.43 (m, 1H), 9.07 (dd, J=4.2, 1.4 Hz, 1H), 8.50-8.40 (m, 2H), 7.93 (br s, 3H), 7.87-7.73 (m, 2H), 7.18 (br d, J=1.0 Hz, 1H), 4.47-4.28 (m, 2H), 3.79 (dd, J=14.6, 6.3 Hz, 1H), 3.53 (m, 1H), 3.31 (m, 1H), 3.00 (m, 1H), 2.43-2.30 (m, 4H), 2.04 (m, 1H), 1.93-1.73 (m, 2H); LCMS (ES⁺) Method 2A: m/z 487 (M+H)⁺, RT 2.14 min.

### Example 93: 4-((6-(7-(aminomethyl)-7-(4-methylthiazol-2-yl)-3-azabicyclo[4.1.0]heptan-3-yl)-1H-pyrazolo[3,4-b]pyrazin-3-yl)thio)-3-chloropyridin-2-amine (Trifluoro Acetate Salt)

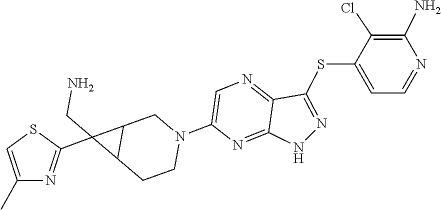 [SMILES: Cc1csc(C2(CN)C3CCN(c4cnc5c(Sc6ccnc(N)c6Cl)n[nH]c5n4)CC32)n1]

A mixture of benzyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in the synthesis of Example 77 step 1; 17 mg, 0.03 mmol) and potassium 2-amino-3-chloropyridine-4-thiolate (prepared by using the procedure described for Intermediate 42 step 3 using tBuOK, 16 mg, 0.078 mmol) in 1,4-dioxane (1 mL), DIPEA (18 uL, 0.100 mmol), XantPhos (1.5 mg, 0.003 mmol) and Pd₂(dba)₃ (4.76 mg, 0.010 mmol) were sequentially added. The reaction mixture was stirred for 3 h at 110° C. The reaction was diluted with EtOAc (1 mL), washed with H₂O (3×1 mL) and the collected organic layers were concentrated to afford crude benzyl ((3-(3-((2-amino-3-chloropyridin-4-yl)thio)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate, which was directly treated with conc. HCl (37%; 780 uL). The residue was purified by preparative HPLC (5-45% MeCN/H₂O+0.1% TFA) to obtain the title compound as a yellow solid (TFA salt; 2 mg, 14% over two steps). LCMS (ES⁺) Method 2: m/z 500, 502 (M+H)⁺, RT 1.82 min.

### Example 94: (3-(3-(2,3-dichlorophenyl)-1H-pyrazolo[3,4-b]pyrazin-6-yl)-7-(thiophen-3-yl)-3-azabicyclo [4.1.0]heptan-7-yl)methanamine (Trifluoro Acetate Salt)

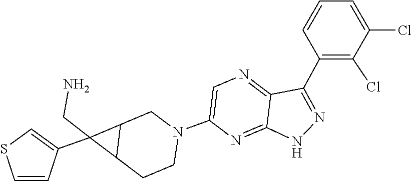 [SMILES: NCC1(c2ccsc2)C2CCN(c3cnc4c(-c5cccc(Cl)c5Cl)n[nH]c4n3)CC21]

### Step 1: 2-((3-(3-iodo-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione

A mixture of Intermediate 44 (52 mg, 0.14 mmol), Intermediate 37 (51 mg, 0.14 mmol) in dry DMF (0.8 mL) was treated with DIPEA (0.12 mL, 0.68 mmol) and heated under MW irradiation to 85° C. for 4 h. The reaction was diluted with EtOAc and the organic phase was washed with H₂O, dried over Na₂SO₄, filtered and concentrated in vacuo. The residue was purified by flash chromatography (0-30% EtOAc in petroleum ether) to afford the title compound as an off-white solid (69 mg, 76%). LCMS (ES⁺) Method 1A: m/z 667 (M+H)⁺, RT 2.62 min.

### Step 2: 2-((3-(3-(2,3-dichlorophenyl)-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione

2-((3-(3-iodo-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione (56 mg, 0.09 mmol) and 2,3-dichlorophenylboronic acid (16 mg, 0.08 mmol) were reacted according to the procedure described in the synthesis of Example 77 step 2. The mixture was diluted with toluene (2 mL) and washed with H₂O (3×2 mL). The collected organic layers were concentrated under reduced pressure, affording the desired compound was used in the next step without further purification (56 mg). LCMS (ES⁺) Method 1A: m/z 685, 687 (M+H)⁺, RT 2.52 min.

### Step 3: (3-(3-(2,3-Dichlorophenyl)-1H-pyrazolo[3,4-b]pyrazin-6-yl)-7-(thiophen-3-yl)-3-azabicyclo [4.1.0]heptan-7-yl)methanamine

2-((3-(3-(2,3-dichlorophenyl)-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione (56 mg, 0.08 mmol) was dissolved in MeOH (2 mL) and treated with HCl (4N in dioxane 1 mL, 4.0 mmol). The reaction was stirred at rt for 1 h then the volatiles were removed in vacuo. The residue was dissolved in EtOAc and NaHCO₃ sol. sat., then the organic layer was dried over Na₂SO₄, filtered and evaporated in vacuo. The crude was dissolved in MeOH (2 mL), treated with N₂H₄·H₂O (20 uL, 0.245 mmol) and stirred at rt for 2 h. The volatiles were removed under reduced pressure and the residue was dissolved in EtOAc and H₂O and the aqueous layer was extracted with EtOAc (×3). The combined organic extracts were concentrated and purified by flash chromatography (C18; 10-50% MeCN/H₂O+0.1% TFA) to obtain the title compound (TFA salt; 8.4 mg, 18%). ¹H NMR (300 MHz, DMSO-d₆+TFA) δ 8.39 (s, 1H), 7.81-7.61 (m, 5H), 7.59-7.45 (m, 2H), 7.41 (m, 1H), 7.16 (m, 1H), 4.31 (m, 2H), 3.71 (m, 1H), 3.33 (m, 1H), 3.17 (m, 1H), 2.93 (m, 1H), 2.29 (m, 1H), 1.85 (m, 1H), 1.73 (m, 1H), 1.57 (m, 1H); LCMS (ES⁺) Method 2A: m/z 471 (M+H)⁺, RT 2.70 min.

### Example 95: (7-(thiophen-3-yl)-3-(3-(2-(trifluoromethyl)pyridin-4-yl)-1H-pyrazolo[3,4-b]pyrazin-6-yl)-3-azabicyclo[4.1.0]heptan-7-yl)methanamine (Trifluoro Acetate Salt)

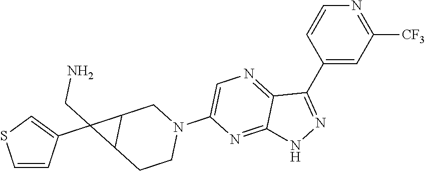 [SMILES: NCC1(c2ccsc2)C2CCN(c3cnc4c(-c5ccnc(C(F)(F)F)c5)n[nH]c4n3)CC21]

### Step 1: 2-((3-(1-(tetrahydro-2H-pyran-2-yl)-3-(2-(trifluoromethyl)pyridin-4-yl)-1H-pyrazolo[3,4-b]pyrazin-6-yl)-7-(thiophen-3-yl)-3-azabicyclo[4.1.0]heptan-7-yl)methyl)isoindoline-1,3-dione

2-((3-(3-iodo-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione (prepared as described in the synthesis of Example 94 step 1; 25 mg, 0.038 mmol) and (2-(trifluoromethyl)pyridin-4-yl)boronic acid (14 mg, 0.07 mmol) were reacted according to the procedure described in the synthesis of Example 77 step 2. The residue was purified by flash chromatography (0-50% EtOAc in petroleum ether) to give the title compound as off-white solid (12 mg, 47%). LCMS (ES⁺) Method 1A: m/z 686 (M+H)⁺, RT 2.44 min.

### Step 2: (7-(thiophen-3-yl)-3-(3-(2-(trifluoromethyl)pyridin-4-yl)-1H-pyrazolo[3,4-b]pyrazin-6-yl)-3-azabicyclo[4.1.0]heptan-7-yl)methanamine

(7-(Thiophen-3-yl)-3-(3-(2-(trifluoromethyl)pyridin-4-yl)-1H-pyrazolo[3,4-b]pyrazin-6-yl)-3-azabicyclo[4.1.0]heptan-7-yl)methanamine was prepared from 2-((3-(1-(tetrahydro-2H-pyran-2-yl)-3-(2-(trifluoromethyl)pyridin-4-yl)-1H-pyrazolo[3,4-b]pyrazin-6-yl)-7-(thiophen-3-yl)-3-azabicyclo[4.1.0]heptan-7-yl)methyl)isoindoline-1,3-dione (12 mg, 0.017 mmol) according to the procedure described in the synthesis of Example 94 step 3. The reaction was directly purified by preparative HPLC (10-60% MeCN/H₂O+0.1% TFA) to obtain the title compound (TFA salt; 9.6 mg, 96%). ¹H NMR (300 MHz, DMSO-d₆+TFA) δ 8.88 (d, J=5.1 Hz, 1H), 8.73 (br s, 1H), 8.56 (s, 1H), 8.52 (m, 1H), 7.71 (br s, 3H), 7.55 (dd, J=4.9, 2.9 Hz, 1H), 7.41 (m, 1H), 7.15 (dd, J=5.1, 1.3 Hz, 1H), 4.33 (m, 2H), 3.77 (m, 1H), 3.32 (m, 1H), 3.14 (m, 1H), 2.97 (m, 1H), 2.31 (m, 1H), 1.86 (m, 1H), 1.72 (m, 1H), 1.57 (m, 1H); LCMS (ES⁺) Method 2A: m/z 472 (M+H)⁺, RT 2.69 min.

### Example 96: (7-(thiophen-3-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine (Trifluoro Acetate Salt)

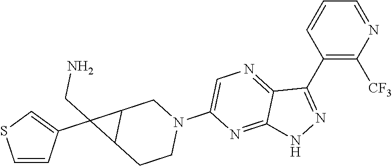 [SMILES: NCC1(c2ccsc2)C2CCN(c3cnc4c(-c5cccnc5C(F)(F)F)n[nH]c4n3)CC21]

### Step 1: 2-((3-(1-(tetrahydro-2H-pyran-2-yl)-3-(2-(trifluoromethyl)pyridin-3-yl)-1H-pyrazolo[3,4-b]pyrazin-6-yl)-7-(thiophen-3-yl)-3-azabicyclo[4.1.0]heptan-7-yl)methyl)isoindoline-1,3-dione

2-((3-(3-iodo-1-(tetrahydro-2H-pyran-2-yl)-1H-pyrazolo[3,4-b]pyrazin-6-yl)-7-(thiophen-3-yl)-3-azabicyclo[4.1.0]heptan-7-yl)methyl)isoindoline-1,3-dione (prepared as described in the synthesis of Example 94 step 1; 25 mg, 0.038 mmol) and (2-(trifluoromethyl)pyridin-3-yl)boronic acid (14 mg, 0.07 mmol) were reacted according to the procedure described in the synthesis of Example 77 step 2. The mixture was diluted with toluene (2 mL) and washed with H₂O (3×2 mL). The collected organic layers were concentrated in vacuo, affording the desired compound which was used in the next step without further purification (25 mg). LCMS (ES⁺) Method 1A: m/z 686 (M+H)⁺, RT 2.64 min.

### Step 2: (7-(thiophen-3-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine

(7-(Thiophen-3-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine was prepared from 2-((3-(1-(tetrahydro-2H-pyran-2-yl)-3-(2-(trifluoromethyl)pyridin-3-yl)-1H-pyrazolo[3,4-b]pyrazin-6-yl)-7-(thiophen-3-yl)-3-azabicyclo[4.1.0]heptan-7-yl)methyl)isoindoline-1,3-dione (12 mg, 0.017 mmol) according to the procedure described in the synthesis of Example 94 step 3. The reaction was directly purified by preparative HPLC (5-50% MeCN/H₂O+0.1% TFA) to obtain the title compound (TFA salt; 5.3 mg, 53%). ¹H NMR (300 MHz, DMSO-d₆+TFA) δ 8.85 (br d, J=4.9 Hz, 1H), 8.40 (s, 1H), 8.27 (br d, J=8.0, 1H), 7.87 (dd, J=7.9, 4.7 Hz, 1H), 7.71 (br s, 3H), 7.56 (dd, J=5.0, 2.9 Hz, 1H), 7.41 (m, 1H), 7.16 (dd, J=5.0, 1.2 Hz, 1H), 4.31 (m, 2H), 3.72 (m, 1H), 3.34 (m, 1H), 3.17 (m, 1H), 2.94 (m, 1H), 2.31 (m, 1H), 1.85 (m, 1H), 1.73 (m, 1H), 1.57 (m, 1H); LCMS (ES⁺) Method 2A: m/z 472 (M+H)⁺, RT 2.26 min.

### Example 97: (3-(3-(2,3-dichlorophenyl)-1H-pyrazolo[3,4-b]pyrazin-6-yl)-7-(1-methyl-1H-pyrazol-5-yl)-3-azabicyclo[4.1.0]heptan-7-yl)methanamine (Trifluoro Acetate Salt)

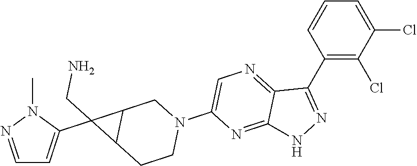 [SMILES: Cn1nccc1C1(CN)C2CCN(c3cnc4c(-c5cccc(Cl)c5Cl)n[nH]c4n3)CC21]

### Step 1: benzyl ((3-(3-iodo-1-(tetrahydro-2H-pyran-2-yl)-1H-pyrazolo[3,4-b]pyrazin-6-yl)-7-(1-methyl-1H-pyrazol-5-yl)-3-azabicyclo[4.1.0]heptan-7-yl)methyl)carbamate

Intermediate 34 (24 mg, 0.06 mmol) was reacted with Intermediate 44 (24 mg, 0.06 mmol) according to the procedure described in the synthesis of Example 77 step 1. The crude title compound was used in the next step without further purification (45 mg). LCMS (ES⁺) Method 1A: m/z 669 (M+H)⁺, RT 2.14 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(1-methyl-1H-pyrazol-5-yl)-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-(1-methyl-1H-pyrazol-5-yl)-3-azabicyclo[4.1.0]heptan-7-yl)methyl)carbamate (20 mg, 0.03 mmol) and (2,3-dichlorophenyl)boronic acid (11 mg, 0.06 mmol) were reacted according to the procedure described in the synthesis of Example 77 step 2. The residue was purified by flash chromatography (0-100% EtOAc in petroleum ether) to give the title compound as a yellow oil (12 mg, 58% over two steps). LCMS (ES⁺) Method 1A: m/z 687, 689 (M+H)⁺, RT 2.47 min.

### Step 3: (3-(3-(2,3-dichlorophenyl)-1H-pyrazolo[3,4-b]pyrazin-6-yl)-7-(1-methyl-1H-pyrazol-5-yl)-3-azabicyclo[4.1.0]heptan-7-yl)methanamine

(3-(3-(2,3-Dichlorophenyl)-1H-pyrazolo[3,4-b]pyrazin-6-yl)-7-(1-methyl-1H-pyrazol-5-yl)-3-azabicyclo[4.1.0]heptan-7-yl)methanamine was prepared from benzyl ((3-(3-(2,3-dichlorophenyl)-1-(tetrahydro-2H-pyran-2-yl)-1H-pyrazolo[3,4-b]pyrazin-6-yl)-7-(1-methyl-1H-pyrazol-5-yl)-3-azabicyclo[4.1.0]heptan-7-yl)methyl)carbamate (20 mg, 0.03 mmol) and treated according to the procedure described in the synthesis of Example 77 step 3. The reaction was directly purified by preparative HPLC (5-45% MeCN/H₂O+0.1% TFA) to obtain the title compound (TFA salt; 2 mg, 14%). ¹H NMR (300 MHz, DMSO-d₆) δ 8.42 (s, 1H), 7.79-7.65 (m, 5H), 7.54-7.38 (m, 2H), 6.26 (d, J=1.8 Hz, 1H), 4.53-4.45 (m, 1H), 4.39-4.31 (m, 1H), 3.89 (s, 3H), 3.75-3.66 (m, 1H), 3.21-3.04 (m, 2H), 2.96-2.85 (m, 1H), 2.39-2.31 (m, 1H), 1.91-1.76 (m, 2H), 1.69-1.59 (m, 1H); LCMS (ES⁺) Method 2A: m/z 469, 471 (M+H)⁺, RT 2.36 min.

### Example 98: (3-(3-(2,3-dichlorophenyl)-1H-pyrazolo[3,4-b]pyrazin-6-yl)-7-(1-methyl-1H-pyrazol-3-yl)-3-azabicyclo[4.1.0]heptan-7-yl)methanamine (Trifluoro Acetate Salt)

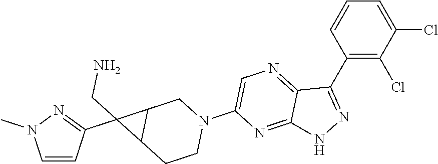 [SMILES: Cn1ccc(C2(CN)C3CCN(c4cnc5c(-c6cccc(Cl)c6Cl)n[nH]c5n4)CC32)n1]

### Step 1: benzyl ((3-(3-iodo-1-(tetrahydro-2H-pyran-2-yl)-1H-pyrazolo[3,4-b]pyrazin-6-yl)-7-(1-methyl-1H-pyrazol-3-yl)-3-azabicyclo[4.1.0]heptan-7-yl)methyl)carbamate

Intermediate 33 (17 mg, 0.04 mmol) was reacted with Intermediate 44 (17 mg, 0.04 mmol) according to the procedure described in the synthesis of Example 77 step 1. The crude title compound was used in the next step without further purification (30 mg). LCMS (ES⁺) Method 1A: m/z 669 (M+H)⁺, RT 2.27 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(1-methyl-1H-pyrazol-3-yl)-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-(1-methyl-1H-pyrazol-3-yl)-3-azabicyclo[4.1.0]heptan-7-yl)methyl)carbamate (30 mg, 0.04 mmol) and (2,3-dichlorophenyl)boronic acid (17 mg, 0.09 mmol) were reacted according to the procedure described in the synthesis of Example 77 step 2. The residue was purified by flash chromatography (0-100% EtOAc in petroleum ether) to give the title compound as a yellow oil (6 mg, 19%, over two steps). LCMS (ES⁺) Method 1A: m/z 687, 689 (M+H)⁺, RT 2.58 min.

### Step 3: (3-(3-(2,3-dichlorophenyl)-1H-pyrazolo[3,4-b]pyrazin-6-yl)-7-(1-methyl-1H-pyrazol-3-yl)-3-azabicyclo[4.1.0]heptan-7-yl)methanamine

(3-(3-(2,3-dichlorophenyl)-1H-pyrazolo[3,4-b]pyrazin-6-yl)-7-(1-methyl-1H-pyrazol-3-yl)-3-azabicyclo[4.1.0]heptan-7-yl)methanamine was prepared from benzyl ((3-(3-(2,3-dichlorophenyl)-1-(tetrahydro-2H-pyran-2-yl)-1H-pyrazolo[3,4-b]pyrazin-6-yl)-7-(1-methyl-1H-pyrazol-3-yl)-3-azabicyclo[4.1.0]heptan-7-yl)methyl)carbamate (5.5 mg, 0.008 mmol) according to the procedure described in the synthesis of Example 77 step 3. The reaction was directly purified by preparative HPLC (5-50% MeCN/H₂O+0.1% TFA) to obtain the title compound (TFA salt; 2 mg, 53%). ¹H NMR (300 MHz, DMSO-d₆+TFA) δ 8.37 (s, 1H), 7.82-7.66 (m, 5H), 7.61 (d, J=2.2 Hz, 1H), 7.50 (t, J=8.2 Hz, 1H), 6.01 (d, J=2.3 Hz, 1H), 4.29 (br d, J=13.9 Hz, 2H), 3.80 (s, 3H), 3.72 (dd, J=14.4, 6.4 Hz, 1H), 3.42-3.31 (m, 1H), 3.27-3.15 (m, 1H), 3.01-2.88 (m, 1H), 1.90-1.68 (m, 3H), 1.66-1.55 (m, 1H); LCMS (ES⁺) Method 2A: m/z 469, 471 (M+H)⁺, RT 2.52 min.

### Example 99: (3-(3-(2,3-dichlorophenyl)-1H-pyrazolo[3,4-b]pyrazin-6-yl)-7-(isoxazol-3-yl)-3-azabicyclo[4.1.0]heptan-7-yl)methanamine (Trifluoro Acetate Salt)

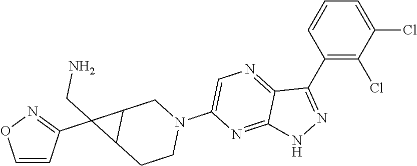 [SMILES: NCC1(c2ccon2)C2CCN(c3cnc4c(-c5cccc(Cl)c5Cl)n[nH]c4n3)CC21]

### Step 1: benzyl ((3-(3-iodo-1-(tetrahydro-2H-pyran-2-yl)-1H-pyrazolo[3,4-b]pyrazin-6-yl)-7-(isoxazol-3-yl)-3-azabicyclo[4.1.0]heptan-7-yl)methyl)carbamate

Intermediate 35 (22 mg, 0.06 mmol) was reacted with Intermediate 44 (23 mg, 0.06 mmol) according to the procedure described in the synthesis of Example 77 step 1. The crude title compound was used in the next step without further purification (38 mg). LCMS (ES⁺) Method 1A: m/z 656 (M+H)⁺, RT 2.32 min.

### Step 2: benzyl ((3-(3-(2,3-dichlorophenyl)-1-(tetrahydro-2H-pyran-2-yl)-1H-pyrazolo[3,4-b]pyrazin-6-yl)-7-(293soxazole-3-yl)-3-azabicyclo[4.1.0]heptan-7-yl)methyl)carbamate

Benzyl ((3-(3-iodo-1-(tetrahydro-2H-pyran-2-yl)-1H-pyrazolo[3,4-b]pyrazin-6-yl)-7-(293soxazole-3-yl)-3-azabicyclo[4.1.0]heptan-7-yl)methyl)carbamate (38 mg, 0.06 mmol) and (2,3-dichlorophenyl)boronic acid (22 mg, 0.12 mmol) were reacted according to the procedure described in the synthesis of Example 77 step 2. The residue was purified by flash chromatography (0-100% EtOAc in petroleum ether) to give the title compound as a yellow oil (38 mg, 97%, over two steps). LCMS (ES⁺) Method 1A: m/z 674, 676 (M+H)⁺, RT 2.62 min.

### Step 3: (3-(3-(2,3-dichlorophenyl)-1H-pyrazolo[3,4-b]pyrazin-6-yl)-7-(isoxazol-3-yl)-3-azabicyclo[4.1.0]heptan-7-yl)methanamine

(3-(3-(2,3-Dichlorophenyl)-1H-pyrazolo[3,4-b]pyrazin-6-yl)-7-(isoxazol-3-yl)-3-azabicyclo[4.1.0]heptan-7-yl)methanamine was prepared from benzyl ((3-(3-(2,3-dichlorophenyl)-1-(tetrahydro-2H-pyran-2-yl)-1H-pyrazolo[3,4-b]pyrazin-6-yl)-7-(isoxazol-3-yl)-3-azabicyclo[4.1.0]heptan-7-yl)methyl)carbamate (39 mg, 0.06 mmol) according to the procedure described in the synthesis of Example 91 step 4. The reaction was directly purified by preparative HPLC (5-50% MeCN/H₂O+0.1% TFA) to obtain the title compound (TFA salt; 6 mg, 22%). ¹H NMR (300 MHz, DMSO-d₆+TFA) δ 8.83 (d, J=1.7 Hz, 1H), 8.39 (s, 1H), 7.91 (br s, 3H), 7.75-7.64 (m, 2H), 7.46 (t, J=7.9 Hz, 1H), 6.47 (d, J=1.7 Hz, 1H), 4.43-4.28 (m, 2H), 3.71 (dd, J=14.6, 6.24 Hz, 1H), 3.54-3.41 (m, 1H), 3.32-3.21 (m, 1H), 3.04-2.87 (m, 1H), 2.37-2.30 (m, 1H), 2.07-1.95 (m, 1H), 1.86-1.72 (m, 2H). LCMS (ES⁺) Method 2A: m/z 456, 458 (M+H)⁺, RT 2.43 min.

### Example 100: (3-(3-(2,3-dichlorophenyl)-1H-pyrazolo[3,4-b]pyrazin-6-yl)-3-azabicyclo[4.1.0]heptan-7-yl)methanamine (Trifluoro Acetate Salt)

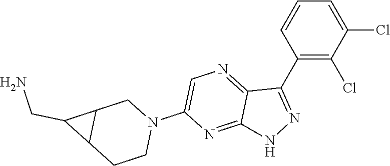 [SMILES: NCC1C2CCN(c3cnc4c(-c5cccc(Cl)c5Cl)n[nH]c4n3)CC12]

### Step 1: 2-((3-(3-iodo-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione

Intermediate 38 (11 mg, 0.04 mmol) was reacted with Intermediate 44 (25 mg, 0.05 mmol) according to the procedure described in the synthesis of Example 77 step 1. The crude title compound was used in the next step without further purification (22 mg). LCMS (ES⁺) Method 1A: m/z 584, 586 (M+H)⁺, RT 2.43 min.

### Step 2: 2-((3-(3-(2,3-dichlorophenyl)-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione

2-((3-(3-iodo-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione (22 mg, 0.039 mmol) and 2,3-dichlorophenylboronic acid (7.4 mg, 0.039 mmol) were reacted according to the procedure described in the synthesis of Example 77 step 2. The residue was purified by flash chromatography (20-100% EtOAc in petroleum ether) to give the title compound (10 mg, 42%, over two steps). LCMS (ES⁺) Method 1A: m/z 603, 605 (M+H)⁺, RT 2.73 min.

### Step 3: (3-(3-(2,3-dichlorophenyl)-1H-pyrazolo[3,4-b]pyrazin-6-yl)-3-azabicyclo[4.1.0]heptan-7-yl)methanamine

(3-(3-(2,3-Dichlorophenyl)-1H-pyrazolo[3,4-b]pyrazin-6-yl)-3-azabicyclo[4.1.0]heptan-7-yl)methanamine was prepared from 2-((3-(3-(2,3-dichlorophenyl)-1-(tetrahydro-2H-pyran-2-yl)-1H-pyrazolo[3,4-b]pyrazin-6-yl)-3-azabicyclo[4.1.0]heptan-7-yl)methyl)isoindoline-1,3-dione (10 mg, 0.017 mmol) according to the procedure described in the synthesis of Example 94 step 3. The reaction was directly purified by preparative HPLC (5-50% MeCN/H₂O+0.1% TFA) to afford the title compound (TFA salt; 6.2 mg, 98%). ¹H NMR (300 MHz, DMSO-d₆) δ 8.31 (s, 1H), 7.59-7.79 (m, 5H), 7.49 (t, J=8.1 Hz, 1H), 4.22 (d, J=13.3 Hz, 1H), 3.85-3.68 (m, 2H), 3.38-3.27 (m, 1H), 2.81-2.66 (m, 2H), 2.14-2.03 (m, 1H), 1.91-1.78 (m, 1H), 1.27-1.12 (m, 2H), 0.89-0.76 (m, 1H). LCMS (ES⁺) Method 2A: m/z 389, 391 (M+H)⁺, RT 2.37 min.

### Example 101: (6-(7-(aminomethyl)-7-(4-methylthiazol-2-yl)-3-azabicyclo[4.1.0]heptan-3-yl)-3-(quinolin-5-yl)-1H-pyrazolo[3,4-b]pyrazin-5-yl)methanol (Trifluoro Acetate Salt)

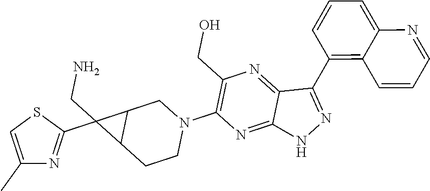 [SMILES: Cc1csc(C2(CN)C3CCN(c4nc5[nH]nc(-c6cccc7ncccc67)c5nc4CO)CC32)n1]

### Step 1: 6-(7-((1,3-dioxoisoindolin-2-yl)methyl)-7-(4-methylthiazol-2-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate

Intermediate 36 (34 mg, 0.087 mmol) and 6-chloro-3-iodo-1-(tetrahydro-2H-pyran-2-yl)-1H-pyrazolo[3,4-b]pyrazin-5-yl)methyl acetate (prepared as reported in the synthesis of Example 55 step 1-5; 40 mg, 0.092 mmol) were reacted according to the procedure described in the synthesis of Example 77 step 1. The crude title compound was used in the next step without further purification (78 mg). LCMS (ES⁺) Method 1A: m/z 754 (M+H)⁺, RT 2.30 min.

### Step 2: (6-(7-((1,3-dioxoisoindolin-2-yl)methyl)-7-(4-methylthiazol-2-yl)-3-azabicyclo[4.1.0]heptan-3-yl)-3-(quinolin-5-yl)-1-(tetrahydro-2H-pyran-2-yl)-1H-pyrazolo[3,4-b]pyrazin-5-yl)methyl acetate

(6-(7-((1,3-Dioxoisoindolin-2-yl)methyl)-7-(4-methylthiazol-2-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate (43 mg, 0.057 mmol) and quinoline-5-boronic acid (20 mg, 0.114 mmol) were reacted according to the procedure described in the synthesis of Example 77 step 2. The residue was purified by flash chromatography (50-100% EtOAc in petroleum ether) to give the title compound as a yellow oil (31 mg, 72% over two steps). LCMS (ES⁺) Method 1A: m/z 755 (M+H)⁺, RT 1.84 min.

### Step 3:(6-(7-(aminomethyl)-7-(4-methylthiazol-2-yl)-3-azabicyclo[4.1.0]heptan-3-yl)-3-(quinolin-5-yl)-1H-pyrazolo[3,4-b]pyrazin-5-yl)methanol

(6-(7-((1,3-Dioxoisoindolin-2-yl)methyl)-7-(4-methylthiazol-2-yl)-3-azabicyclo[4.1.0]heptan-3-yl)-3-(quinolin-5-yl)-1-(tetrahydro-2H-pyran-2-yl)-1H-pyrazolo[3,4-b]pyrazin-5-yl)methyl acetate (31 mg, 0.04 mmol) was suspended in EtOH (1 mL) and N₂H₄·H₂O (0.06 mL, 1.23 mmol) was added. The reaction mixture was stirred at rt for 5 h, then was filtered and solid washed with DCM (×2). The mother liquors were concentrated under reduced pressure and the residue was taken up in DCM and filtered again. Volatiles were removed in vacuo to obtain a yellow foam (27 mg). The product was dissolved again in DCM (0.7 mL) and HCl (4N in 1,4-dioxane; 0.25 mL, 1 mmol) was added. The reaction mixture was stirred at rt for 1 h, then solvent was removed under reduced pressure. The residue was directly purified by preparative HPLC (0-35% MeCN/H₂O+0.1% TFA) to obtain the title compound (TFA salt; 13 mg, 52%) as a yellow solid. ¹H NMR (300 MHz, DMSO-d₆+TFA) δ 10.09 (d, J=8.7 Hz, 1H), 9.41 (dd, J=5.2, 1.4 Hz, 1H), 8.92 (dd, J=6.8, 1.6 Hz, 1H), 8.40-8.24 (m, 2H), 8.18 (dd, J=8.7, 5.2 Hz, 1H), 7.98 (br s, 3H), 7.18 (br d, J=1.0 Hz, 1H), 4.70 (s, 2H), 4.01-3.85 (m, 2H), 3.84-3.65 (m, 3H), 2.77 (m, 1H), 2.44-2.25 (m, 4H), 2.15 (m, 1H), 2.01 (m, 1H), 1.95-1.82 (m, 1H); LCMS (ES⁺) Method 2A: m/z 499 (M+H)⁺, RT 1.77 min.

### Example 102: (6-(7-(aminomethyl)-7-(4-methylthiazol-2-yl)-3-azabicyclo[4.1.0]heptan-3-yl)-3-(2,3-dichlorophenyl)-1H-pyrazolo[3,4-b]pyrazin-5-yl)methanol (Trifluoro Acetate Salt)

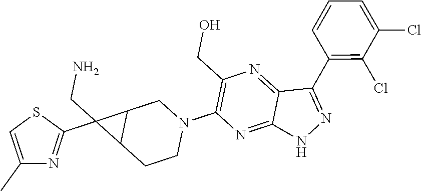 [SMILES: Cc1csc(C2(CN)C3CCN(c4nc5[nH]nc(-c6cccc(Cl)c6Cl)c5nc4CO)CC32)n1]

### Step 1: (3-(2,3-dichlorophenyl)-6-(7-((1,3-dioxoisoindolin-2-yl)methyl)-7-(4-methylthiazol-2-yl)-3-azabicyclo[4.1.0]heptan-3-yl)-1-(tetrahydro-2H-pyran-2-yl)-1H-pyrazolo[3,4-b]pyrazin-5-yl)methyl acetate

(6-(7-((1,3-Dioxoisoindolin-2-yl)methyl)-7-(4-methylthiazol-2-yl)-3-azabicyclo[4.1.0]heptan-3-yl)-3-iodo-1-(tetrahydro-2H-pyran-2-yl)-1H-pyrazolo[3,4-b]pyrazin-5-yl)methyl acetate (36 mg, 0.048 mmol) and 2,3-dichlorophenylboronic acid (18 mg, 0.096 mmol) were reacted according to the procedure described in the synthesis of Example 77 step 2. The residue was purified by flash chromatography (30-70% EtOAc in petroleum ether) to give the title compound as a yellow oil (25 mg, 68%). LCMS (ES⁺) Method 1A: m/z 772, 774 (M+H)⁺, RT 2.58 min.

### Step 2: 6-(7-(aminomethyl)-7-(4-methylthiazol-2-yl)-3-azabicyclo[4.1.0]heptan-3-yl)-3-(2,3-dichlorophenyl)-1H-pyrazolo[3,4-b]pyrazin-5-yl)methanol

6-(7-(Aminomethyl)-7-(4-methylthiazol-2-yl)-3-azabicyclo[4.1.0]heptan-3-yl)-3-(2,3-dichlorophenyl)-1H-pyrazolo[3,4-b]pyrazin-5-yl)methanol was prepared (3-(2,3-dichlorophenyl)-6-(7-((1,3-dioxoisoindolin-2-yl)methyl)-7-(4-methylthiazol-2-yl)-3-azabicyclo[4.1.0]heptan-3-yl)-1-(tetrahydro-2H-pyran-2-yl)-1H-pyrazolo[3,4-b]pyrazin-5-yl)methyl acetate (25 mg, 0.032 mmol) according to the procedure described in the synthesis of Example 101 step 3. The residue was directly purified by preparative HPLC (5-55% MeCN/H₂O+0.1% TFA) to obtain the title compound as a yellow solid (TFA salt; 5 mg, 25%). ¹H NMR (300 MHz, DMSO-d₆+TFA) δ 7.94 (br s, 3H), 7.74 (m, 2H), 7.51 (t, J=7.9 Hz, 1H), 7.17 (br d, J=1.0 Hz, 1H), 4.61 (s, 2H), 3.97-3.85 (m, 2H), 3.84-3.62 (m, 3H), 2.79-2.65 (m, 1H), 2.43-2.23 (m, 4H), 2.20-2.05 (m, 1H), 1.98 (m, 1H), 1.92-1.82 (m, 1H); LCMS (ES⁺) Method 2A: m/z 516-518 (M+H)⁺, RT 2.67 min.

### Example 103: 4-(6-(7-(aminomethyl)-7-(4-methylthiazol-2-yl)-3-azabicyclo[4.1.0]heptan-3-yl)-1H-pyrazolo[3,4-b]pyrazin-3-yl)naphthalen-1-yl sulfurofluoridate (Trifluoro Acetate Salt)

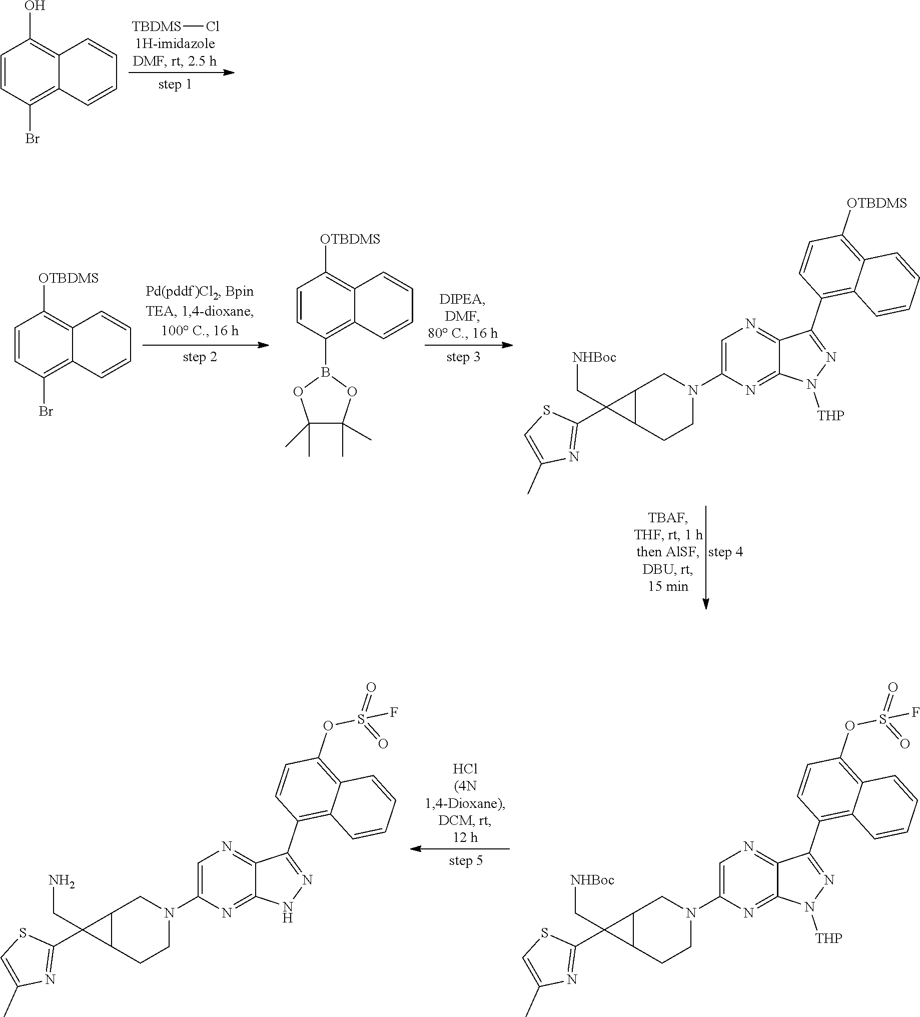

### Step 1: ((4-bromonaphthalen-1-yl)oxy)(tert-butyl)dimethylsilane

To a solution of 4-bromonaphthalen-1-ol (200 mg, 0.90 mmol) and 1H-imidazole (74.5 mg, 1.09 mmol) in dry DMF (2 mL, 0.025 mol) at 0° C., TBDMS-Cl (149 mg, 0.99 mmol) was added. The reaction mixture was stirred at rt for 2.5 h, then was diluted with Et₂O (2 mL) and H₂O (2 mL). The aqueous layer was extracted again with Et₂O (3×3 mL), then combined organic layers were washed with H₂O (×2) and brine (×2), dried over Na₂SO₄, filtered and concentrated in vacuo. The resulting crude was purified by flash chromatography (100% petroleum ether) to afford the title compound as white crystals (273 mg, 90%). ¹H NMR (300 MHz, CDCl₃) δ 8.16 (m, 2H), 7.62-7.44 (m, 3H), 6.71 (d, J=8.2 Hz, 1H), 1.07 (s, 9H), 0.26 (s, 6H)

### Step 2: tert-butyldimethyl((4-(4,4,5,5-tetramethyl-1,3,2-dioxaborolan-2-yl)naphthalen-1-yl)oxy)silane

A mixture of ((4-bromonaphthalen-1-yl)oxy)(tert-butyl)dimethylsilane (140 mg, 0.42 mmol), B₂pin₂ (211 mg, 0.83 mmol), Pd(dppf)Cl₂·DCM (42 mg, 0.05 mmol) and TEA (0.23 mL, 1.66 mmol) in degassed 1,4-dioxane (1.3 mL) was stirred at 100° C. for 16 h. The black suspension was diluted with H₂O (1 mL) and DCM (1 mL), the two phases were separated and the aqueous layer was extracted again with DCM (3×3 mL). The combined organic extracts were washed with brine (×3), dried over Na₂SO₄, filtered and concentrated. The resulting crude was purified by flash chromatography (0-5% EtOAc in petroleum ether) to afford the title compound as a yellow solid (105 mg, 66%). ¹H NMR (300 MHz, CDCl₃) δ 8.72 (br d, J=8.4 Hz, 1H), 8.19 (m, 1H), 7.94 (d, J=7.6 Hz, 1H), 7.55-7.38 (m, 2H) 6.83 (d, J=7.6 Hz, 1H), 1.39 (s, 12H), 1.07 (s, 9H), 0.27 (s, 6H); LCMS (ES⁺) Method 3: m/z 385 (M+H)⁺, RT 2.77 min.

### Step 3: tert-butyl ((3-(3-(4-((tert-butyldimethylsilyl)oxy)naphthalen-1-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate

Tert-butyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in the synthesis of Example 91 step 1; 20 mg, 0.031 mmol) and tert-butyldimethyl((4-(4,4,5,5-tetramethyl-1,3,2-dioxaborolan-2-yl)naphthalen-1-yl)oxy)silane (23.6 mg, 0.061 mmol) were prepared according to the procedure described in the synthesis of Example 77 step 2. The residue was purified by flash chromatography (10-50% EtOAc in petroleum ether) to afford the title compound as a yellow oil (10 mg, 42%). LCMS (ES⁺) Method 3: m/z 782, 783 (M+H)⁺, RT 2.67 min.

### Step 4: 4-(6-(7-(((tert-butoxycarbonyl)amino)methyl)-7-(4-methylthiazol-2-yl)-3-azabicyclo[4.1.0]heptan-3-yl)-1-(tetrahydro-2H-pyran-2-yl)-1H-pyrazolo[3,4-b]pyrazin-3-yl)naphthalen-1-yl sulfurofluoridate

Tert-butyl ((3-(3-(4-((tert-butyldimethylsilyl)oxy)naphthalen-1-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (10 mg, 0.010 mmol) was dissolved in THF (0.5 mL) and TBAF (1M solution in THF; 0.03 mL, 0.030 mmol) was added. The resulting dark-red solution was stirred at rt for 1 h, then H₂O and saturated NH₄Cl solution were added, and the mixture was extracted with EtOAc (×3). The organic layer was dried over Na₂SO₄, filtered and concentrated in vacuo to obtain tert-butyl ((3-(3-(4-hydroxynaphthalen-1-yl)-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate as yellow oil (11 mg). The residue was dissolved in dry THF (0.5 mL) and AISF (4.8 mg, 0.020 mmol), DBU (0.004 mL, 0.030 mmol) were added, then the reaction mixture was stirred at rt for 15 min. EtOAc (1 mL) and H₂O (1 mL) were added, phases were separated, and the organic layer was washed with 0.5N HCl (×3) and brine (×2). The organic phase was dried over Na₂SO₄, filtered and concentrated in vacuo to afford crude title compound (12 mg), which was used in the next step without further purification. LCMS (ES⁺) Method 3: m/z 750 (M+H)⁺, RT 2.20 min.

### Step 5: 4-(6-(7-(Aminomethyl)-7-(4-methylthiazol-2-yl)-3-azabicyclo[4.1.0]heptan-3-yl)-1H-pyrazolo[3,4-b]pyrazin-3-yl)naphthalen-1-yl sulfurofluoridate

4-(6-(7-(Aminomethyl)-7-(4-methylthiazol-2-yl)-3-azabicyclo[4.1.0]heptan-3-yl)-1H-pyrazolo[3,4-b]pyrazin-3-yl)naphthalen-1-yl sulfurofluoridate was prepared starting from crude 4-(6-(7-(((tert-butoxycarbonyl)amino)methyl)-7-(4-methylthiazol-2-yl)-3-azabicyclo[4.1.0]heptan-3-yl)-1-(tetrahydro-2H-pyran-2-yl)-1H-pyrazolo[3,4-b]pyrazin-3-yl)naphthalen-1-yl sulfurofluoridate (9.6 mg; 0.013 mmol) according to the procedure described for the synthesis of Example 91 step 4. The residue was purified by preparative HPLC (15-65% MeCN/H₂O+0.1% TFA) to obtain the title compound as yellow solid (TFA salt; 4.5 mg, 51% over two steps). ¹H NMR (300 MHz, DMSO-d₆+TFA) δ 8.89 (d, J=8.5 Hz, 1H), 8.46 (s, 1H), 8.30 (d, J=8.2 Hz, 1H), 8.17 (d, J=8.3 Hz, 1H), 8.05-7.88 (m, 4H), 7.87-7.71 (m, 2H), 7.18 (br d, J=1.0 Hz, 1H), 4.49-4.27 (m, 2H), 3.78 (dd, J=14.5, 6.3 Hz, 1H), 3.52 (m, 1H), 3.32 (m, 1H), 3.00 (m, 1H), 2.44-2.30 (m, 4H), 2.04 (m, 1H), 1.93-1.75 (m, 2H); LCMS (ES⁺) Method 2: m/z 566 (M+H)⁺, RT 3.09 min.

### Example 104: 6-((1R,5S,6s)-6-(aminomethyl)-6-phenyl-3-azabicyclo[3.1.0]hexan-3-yl)-3-((2-(trifluoromethyl)pyridin-3-yl)thio)pyrazin-2-amine (Trifluoro Acetate Salt)

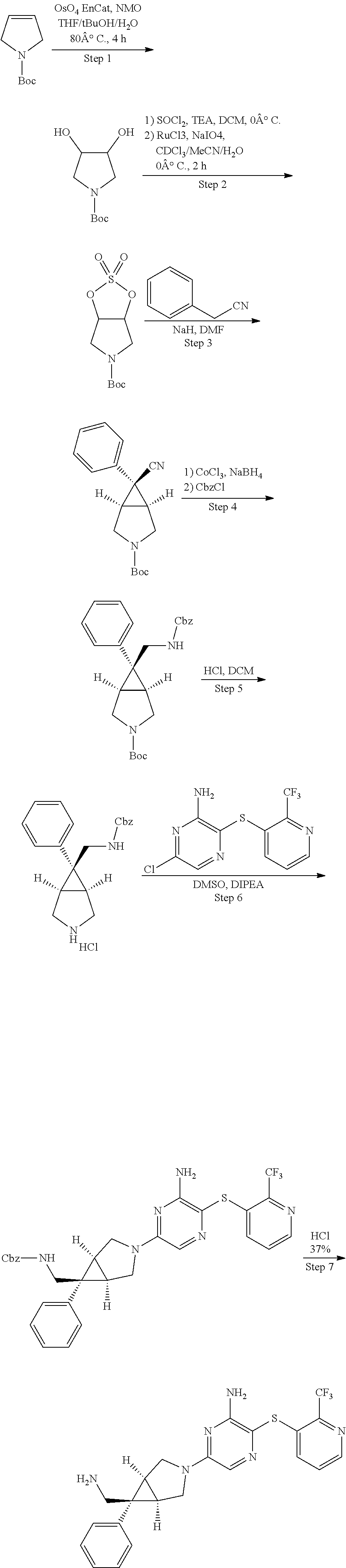

### Step 1: tert-butyl 3,4-dihydroxypyrrolidine-1-carboxylate

OsO₄ En-Cat® (500 mg, 0.10 mmol) was added to a solution of tert-butyl 2,5-dihydro-1H-pyrrole-1-carboxylate (450 mg, 2.66 mmol) and 4-methylmorpholine N-oxide (475 mg, 3.99 mmol) in THF (2.5 mL), t-BuOH (1.7 mL) and H₂O (0.7 mL). The mixture was stirred at 80° C. for 4 h, then OsO₄ En-Cat® was filtered and washed with THF and H₂O. Solvents were removed under reduced pressure, EtOAc was added and the organic phase was washed with brine (×2), dried over Na₂SO₄, filtered and concentrated in vacuo. The resulting crude was purified by flash chromatography (50-100% of EtOAc in petroleum ether) to obtain the title compound as a colourless oil (486 mg, 90%). LCMS (ES⁺) Method 1A: m/z 204 (M+H)⁺, RT 1.04 min.

### Step 2: tert-butyl tetrahydro-5H-[1,3,2]dioxathiolo[4,5-c]pyrrole-5-carboxylate 2,2-dioxide

TEA (1.33 mL, 9.56 mmol) and thionyl chloride (0.26 mL, 3.59 mmol) were added to a solution of tert-butyl 3,4-dihydroxypyrrolidine-1-carboxylate (486 mg, 2.39 mmol) in DCM (12 mL) cooled to 0° C. The mixture was stirred at this temperature for 15 min. After quenching the reaction by addition of H₂O (0.6 mL), the mixture was diluted with Et₂O and additional H₂O. The organic phase was washed with H₂O, NaHCO₃ sat. sol. and brine, dried over Na₂SO₄, filtered and concentrated in vacuo to obtain a dark solid (600 mg). The residue was dissolved in CHCl₃ (7 mL), MeCN (7 mL) and H₂O (10.5 mL), then ruthenium(III) chloride (14.9 mg, 0.07 mmol) and sodium periodate (1024 mg, 4.79 mmol) were added at 0° C. The mixture was stirred at this temperature for 2 h, then was diluted with Et₂O and washed with H₂O and brine. The organic layer was dried over Na₂SO₄, filtered and concentrated in vacuo. The resulting crude was purified by flash chromatography (40-60% of EtOAc in petroleum ether) to afford the title compound as a white solid (546 mg, 86%). LCMS (ES⁺) Method 1A: m/z 266 (M+H)⁺, RT 1.75 min.

### Step 3: tert-butyl (1R,5S,6s)-6-cyano-6-phenyl-3-azabicyclo[3.1.0]hexane-3-carboxylate

Tert-butyl tetrahydro-5H-[1,3,2]dioxathiolo[4,5-c]pyrrole-5-carboxylate 2,2-dioxide (258 mg, 0.87 mmol) and benzyl cyanide (125 mg, 1.07 mmol) were dissolved in dry DMF (2.2 mL) and the resulting solution was added dropwise over a period of 30 min to a suspension of NaH (60% in mineral oil; 97 mg, 2.43 mmol) in dry DMF (1.4 mL) cooled to 0° C. under N₂ flow. The reaction mixture was stirred at 0° C. for 20 min, then was quenched by addition of brine and diluted with EtOAc. The organic layer was washed with H₂O and brine, dried over Na₂SO₄, filtered and concentrated in vacuo. The resulting crude was purified by flash chromatography (10-30% of EtOAc in petroleum ether) to obtain the title compound as a white solid (255 mg, 92%). ¹H NMR (300 MHz, CDCl₃) δ 7.44-7.26 (m, 5H), 4.07-3.89 (dd, J=12.0 and 23.4 Hz, 2H), 3.83-3.69 (m, 2H), 2.40-2.29 (m, 2H), 1.51 (s, 9H); LCMS (ES⁺) Method 1A: m/z 285 (M+H)⁺, RT 2.08 min.

### Step 4: tert-butyl (1R,5S,6s)-6-((((benzyloxy)carbonyl)amino)methyl)-6-phenyl-3-azabicyclo[3.1.0]hexane-3-carboxylate

tert-butyl (1R,5S,6s)-6-cyano-6-phenyl-3-azabicyclo[3.1.0]hexane-3-carboxylate (100 mg, 0.35 mmol) and CoCl₂ (182 mg, 1.40 mmol) were dissolved in MeOH (5 mL) in a pressure flask, and the dark solution was cooled to 0° C. NaBH₄ (560 mg, 14.8 mmol) was added portion wise, the flask was sealed, and the reaction mixture was vigorously stirred overnight at rt. The solvent was removed in vacuo then the residue was dissolved in DCM (10 mL), the solution was cooled to 0° C. and benzyl chloroformate (0.062 mL, 0.40 mmol) and TEA (0.07 mL, 0.49 mmol) were sequentially added. The reaction mixture was stirred at 0° C. for 30 min. DCM and H₂O were added then the organic layer was washed with 5% citric acid solution. The organic phase was dried over Na₂SO₄, filtered and concentrated under reduced pressure. The resulting crude was purified by flash chromatography (0-20% of EtOAc in DCM) affording the title compound as a colorless oil (45 mg, 30%). ¹H NMR (300 MHz, CDCl₃) δ 7.35-7.06 (m, 10H), 4.95 (s, 2H), 4.54-4.35 (br s, 1H), 3.71-3.45 (m, 5H), 3.44-3.29 (m, 1H), 1.96-1.88 (m, 2H), 1.41 (s, 9H); LCMS (ES⁺) Method 1A: m/z 423 (M+H)⁺, RT 2.37 min.

### Step 5: benzyl (((1R,5S,6s)-6-phenyl-3-azabicyclo[3.1.0]hexan-6-yl)methyl)carbamate hydrochloride

Tert-butyl (1R,5S,6s)-6-((((benzyloxy)carbonyl)amino)methyl)-6-phenyl-3-azabicyclo[3.1.0]hexane-3-carboxylate (44 mg, 0.10 mmol) was dissolved in DCM (1 mL) and treated with HCl (4N in dioxane 1 mL, 4.0 mmol). The solution was stirred at rt for 1.5 h, then the solvent was removed in vacuo to afford the title compound that was used in the next step without further purification (42 mg). LCMS (ES⁺) Method 1A: m/z 323 (M+H)⁺, RT 1.37 min.

### Step 6: benzyl (((1R,5S,6s)-3-(6-amino-5-((2-(trifluoromethyl)pyridin-3-yl)thio)pyrazin-2-yl)-6-phenyl-3-azabicyclo[3.1.0]hexan-6-yl)methyl)carbamate

DIPEA (0.06 mL, 0.326 mmol) was added to a solution of Intermediate 41 (20 mg, 0.065 mmol) and benzyl (((1R,5S,6s)-6-phenyl-3-azabicyclo[3.1.0]hexan-6-yl)methyl)carbamate hydrochloride (35 mg, 0.098 mmol) in DMSO (0.5 mL). The reaction mixture was heated at 120° C. for 3.5 h then concentrated and directly purified by preparative HPLC (35-80% H₂O/MeCN+0.1% TFA) to afford the title compound as off-white solid (25 mg, 65%). ¹H NMR (300 MHz, DMSO-d₆) δ 8.50-8.42 (br d, J=4.3 Hz, 1H), 7.60-7.47 (m, 1H), 7.43-7.14 (m, 12H), 6.44-5.94 (br s, 2H), 4.91 (s, 2H), 3.91-3.79 (m, 2H), 3.78-3.66 (m, 2H), 3.35-3.24 (m, 2H), 2.13 (s, 2H); LCMS (ES⁺) Method 1A: m/z 593 (M+H)⁺, RT 2.40 min.

### Step 7: 6-((1R,5S,6s)-6-(aminomethyl)-6-phenyl-3-azabicyclo[3.1.0]hexan-3-yl)-3-((2-(trifluoromethyl)pyridin-3-yl)thio)pyrazin-2-amine

benzyl (((1R,5S,6s)-3-(6-amino-5-((2-(trifluoromethyl)pyridin-3-yl)thio)pyrazin-2-yl)-6-phenyl-3-azabicyclo[3.1.0]hexan-6-yl)methyl)carbamate (25 mg, 0.040 mmol) was dissolved in 37% aq. HCl (0.7 mL) and the resulting yellow solution was stirred at rt overnight. The reaction was directly purified by preparative HPLC (10-50% H₂O/MeCN+0.1% TFA) to afford the title compound as light-yellow solid TFA salt (20 mg, 83%). ¹H NMR (300 MHz, DMSO-d₆) δ 8.52-8.44 (br d, J=4.29 Hz, 1H), 7.82-7.63 (br s, 3H), 7.60-7.53 (m, 1H), 7.52-7.38 (m, 4H), 7.37-7.27 (m, 3H), 6.35-6.22 (br s, 2H), 3.78 (s, 4H), 3.13-3.02 (m, 2H), 2.28 (s, 2H); LCMS (ES⁺) Method 2A: m/z 459 (M+H)⁺, RT 2.48 min.

### Example 105: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-amine (Trifluoro Acetate Salt)

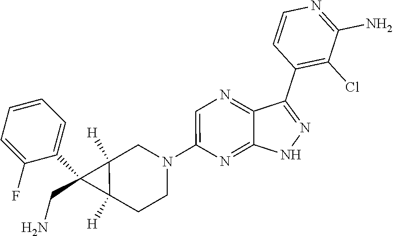 [SMILES: NC[C@]1(c2ccccc2F)[C@@H]2CCN(c3cnc4c(-c5ccnc(N)c5Cl)n[nH]c4n3)C[C@@H]21]

The above example was synthesized using the procedure reported for Example 2 using Intermediate 51 for step 1 and Intermediate 17 for step 2; Example 105 was obtained as a yellow powder (TFA salt; 3.7 mg, 96% pure, 19%). ¹H NMR (400 MHz, DMSO-d₆) δ 13.57 (br s, 1H), 8.46 (s, 1H), 8.01 (d, J=5.3 Hz, 1H), 7.66 (br s, 3H), 7.49 (br t, J=7.8 Hz, 1H), 7.40-7.35 (m, 1H), 7.25-7.19 (m, 2H), 7.07 (d, J=5.0 Hz, 1H), 6.64 (br s, 2H), 4.45 (br d, J=14.5 Hz, 1H), 4.33 (br d, J=12.3 Hz, 1H), 3.75-3.70 (m, 1H), 3.15-3.05 (m, 2H), 2.98-2.90 (m, 1H), 2.36-2.31 (m, 1H), 1.84-1.75 (m, 2H), 1.57-1.52 (m, 1H); LCMS (ES⁺) Method 2: m/z 465 (M+H)⁺, RT 1.16 min.

The following examples were synthesized using the above procedure with Intermediates 51, 44 and the appropriate boronic ester or acid (commercial or, unless otherwise stated, prepared as exemplified for Intermediate 15). The acid used in step 3 is reported below 1 for HCl (37%), 2 for HBr (33% in AcOH)

| Example | Boronic ester or acid | Acid | ¹H NMR (400 MHz) or LCMS (ES⁺) |
|---|---|---|---|
| 118 | 6-(4,4,5,5-tetramethyl- | 1 | (DMSO-d₆) δ 13.36 (br s, 1H), 9.43 (s, 1H), 9.10 (d, J = 1.1 |
| | 1,3,2-dioxaborolan-2- | | Hz, 1H), 8.54 (dd, J = 8.6, 1.5 Hz, 1H), 8.51 (s, 1H), 8.21 (d, |
| | yl)benzo[d]thiazole | | J = 8.8 Hz, 1H), 7.66 (br s, 3H), 7.49 (br t, J = 7.7 Hz, 1H), |
| | (commercial) | | 7.40-7.35 (m, 1H), 7.25-7.19 (m, 2H), 4.45 (br d, J = 14.0 |
| | | | Hz, 1H), 4.35 (br d, J = 13.4 Hz, 1H), 3.78-3.73 (m, 1H), |
| | | | 3.17-3.03 (m, 2H), 2.99-2.93 (m, 1H), 2.39-2.31 (m, 1H), |
| | | | 1.84-1.78 (m, 2H), 1.57-1.53 (m, 1H) |
| 119 | Intermediate 58 | 1 | (DMSO-d₆) δ 13.55 (s, 1H), 8.74 (s, 1H), 8.46 (s, 1H), 8.11 |
| | | | (d, J = 8.8 Hz, 1H), 8.0 (d, J = 8.6 Hz, 1H), 7.65 (br s, 3H), |
| | | | 7.51-7.47 (m, 1H), 7.41-7.34 (m, 1H), 7.27-7.19 (m, 2H), |
| | | | 4.46 (br d, J = 14.5 Hz, 1H), 4.36 (br d, J = 13.6 Hz, 1H), 4.13 |
| | | | (s, 3H), 3.77-3.69 (m, 1H), 3.19-3.05 (m, 2H), 2.99-2.86 |
| | | | (m, 1H), 2.38-2.30 (m, 1H), 1.84-1.75 (m, 2H), 1.59-1.52 |
| | | | (m, 1H) |
| 122 | 4-(4,4,5,5-tetramethyl- | 1 | (DMSO-d₆) δ 13.37 (s, 1H), 10.48 (s, 1H), 8.48 (s, 1H), |
| | 1,3,2-dioxaborolan-2- | | 8.20 (d, J = 8.1 Hz, 1H), 7.66 (br s, 3H), 7.48 (br t, J = 7.7 Hz, |
| | yl)indolin-2-one | | 1H), 7.42-7.32 (m, 2H), 7.23-7.19 (m, 2H), 6.86 (d, J = 7.7 |
| | (commercial) | | Hz, 1H), 4.45 (br d, J = 14.0 Hz, 1H), 4.34 (br d, J = 9.7 Hz, |
| | | | 1H), 3.78 (s, 2H), 3.73 (br dd, J = 14.5 Hz, 6.4 Hz, 1H), 3.17- |
| | | | 3.03 (m, 2H), 2.97-2.90 (m, 1H), 2.38-2.31 (m, 1H), 1.84- |
| | | | 1.75 (m, 2H), 1.56-1.52 (m, 1H) |
| 131 | Intermediate 62 | 1 | (DMSO-d₆) δ 13.51 (s, 1H), 8.76 (d, J = 7.0 Hz, 1H), 8.63 |
| | | | (d, J = 7.0 Hz, 1H), 8.52 (s, 1H), 8.11 (d, J = 2.2 Hz, 1H), 7.66 |
| | | | (br s, 3H), 7.55-7.44 (m, 1H), 7.43-7.33 (m, 2H), 7.29-7.16 |
| | | | (m, 2H), 7.10 (t, J = 7.0 Hz, 1H), 4.47 (br d, J = 13.6 Hz, 2H), |
| | | | 4.41-4.30 1(m, 1H), 3.76 (br dd, J = 14.6, 6.7 Hz, 1H), 3.21- |
| | | | 2.90 (m, 3H), 1.90-1.69 (m, 2H), 1.62-1.50 (m, 1H) |
| 132 | Intermediate 60 | 1 | (DMSO-d₆) δ 13.81 (s, 1H), 9.11 (d, J = 4.4 Hz, 1H), 8.90- |
| | | | 8.85 (m, 1H), 8.55 (s, 1H), 8.46 (d, J = 4.4 Hz, 1H), 7.70- |
| | | | 7.65 (m, 5H), 7.51-7.47 (m, 1H), 7.41-7.36 (m, 1H), 7.25- |
| | | | 7.20 (m, 2H), 4.48 (br d, J = 14.3 Hz, 1H), 4.37 (br d, J = 10.3 |
| | | | Hz, 1H), 3.78 (br dd, J = 14.3, 6.4 Hz, 1H), 3.17-2.95 (m, |
| | | | 3H), 2.38-2.32 (m, 1H), 1.85-1.78 (m, 2H), 1.58-1.54 (m, |
| | | | 1H) |
| 136 | 5-(4,4,5,5-tetramethyl- | 1 | (DMSO-d₆) δ 13.43 (br s, 1H), 8.77 (d, J = 7.5 Hz, 1H), 8.74 |
| | 1,3,2-dioxaborolan-2- | | (s, 1H), 8.52 (s, 1H), 8.04 (d, J = 2.2 Hz, 1H), 7.72-7.69 (m, |
| | yl)pyrazolo[1,5- | | 1H), 7.66 (br s, 3H), 7.48 (br t, J = 7.1 Hz, 1H), 7.40-7.36 |
| | a]pyridine | | (m, 1H), 7.25-7.19 (m, 2H), 6.77 (d, J = 1.5 Hz, 1H), 4.45 |
| | | | (br d, J = 14.3 Hz, 1H), 4.35 (br d, J = 10.5 Hz, 1H), 3.76 (br |
| | | | dd, J = 14.4, 6.5 Hz, 1H), 3.14-2.93 (m, 3H), 2.37-2.32 (m, |
| | | | 1H), 1.84-1.75 (m, 2H), 1.57-1.53 (m, 1H) |
| 137 | 2-(5-(4,4,5,5- | 1 | (MeOH-d₄) δ 9.55 (br s, 1H), 9.07 (br d, J = 3.5 Hz, 1H), |
| | tetramethyl-1,3,2- | | 8.43 (s, 1H), 8.40 (br d, J = 8.1 Hz, 1H), 8.07 (d, J = 7.7 Hz, |
| | dioxaborolan-2- | | 1H), 7.83-7.78 (m, 1H), 7.50-7.46 (m, 1H), 7.44-7.39 (m, |
| | yl)quinolin-8- | | 1H), 7.26-7.18 (m, 2H), 4.66-4.62 (m, 1H), 4.50-4.45 (m, |
| | yl)propan-2-ol | | 1H), 3.87-3.81 (m, 1H), 3.37-3.35 (m, 2H), 3.13-3.05 (m, |
| | | | 1H), 2.54-2.47 (m, 1H), 2.03-1.95 (m, 2H), 1.87 (s, 6H), |
| | | | 1.72-1.69 (m, 1H) |
| 138 | Intermediate 72 | 1 | (DMSO-d₆) δ 13.44 (br s, 1H), 9.43 (br s, 1H), 8.98 (br s, |
| | | | 1H), 8.48 (s, 1H), 8.39 (br d, J = 7.2 Hz, 1H), 7.71 (br s, 4H), |
| | | | 7.54-7.48 (m, 2H), 7.41-7.36 (m, 1H), 7.25-7.19 (m, 2H), |
| | | | 5.04-4.99 (m, 1H), 4.48 (br d, J = 14.7 Hz, 1H), 4.35 (br d, |
| | | | J = 9.9 Hz, 1H), 3.78-3.73 (m, 1H), 3.22-3.05 (m, 2H), 3.01- |
| | | | 2.93 (m, 1H), 2.39-2.32 (m, 1H), 1.85-1.76 (m, 2H), 1.59- |
| | | | 1.52 (m, 1H), 1.45 (d, J = 5.9 Hz, 6H) |
| 139 | Intermediate 73 | 1 | (DMSO-d₆) δ 13.45 (br s, 1H), 9.24 (br d, J = 7.7 Hz, 1H), |
| | | | 9.01 (br s, 1H), 8.47 (s, 1H), 8.25 (br d, J = 7.5 Hz, 1H), |
| | | | 7.69-7.60 (m, 4H), 7.52-7.47 (m, 1H), 7.38 (br d, J = 7.7 Hz, |
| | | | 2H), 7.25-7.19 (m, 2H), 4.48 (br d, J = 13.8 Hz, 1H), 4.35 |
| | | | (br d, J = 12.7 Hz, 1H), 3.76-3.71 (m, 1H), 3.36-3.24 (m, |
| | | | 1H), 3.20-3.04 (m, 1H), 2.98-2.92 (m, 1H), 2.38-2.29 (m, |
| | | | 1H), 1.86-1.74 (m, 3H), 1.57-1.53 (m, 1H), 1.16 (d, J = 7.9 |
| | | | Hz, 2H), 0.91 (br d, J = 3.5 Hz, 2H) |
| 141 | 6-(4,4,5,5-tetramethyl- | 1 | (DMSO-d₆) δ 13.44 (br s, 1H), 9.00 (br d, J = 1.5 Hz, 1H), |
| | 1,3,2-dioxaborolan-2- | | 8.95 (br dd, J = 4.2, 1.5 Hz, 1H), 8.72 (br dd, J = 8.8, 1.8 Hz, |
| | | | 1H), 8.55 (br s, 2H), 8.16 (d, J = 8.8 Hz, 1H), 7.66 (br s, 2H), |
| | yl)quinoline | | 7.62 (dd, J = 8.3, 4.4 Hz, 1H), 7.49 (br t, J = 7.0 Hz, 1H), |
| | (commercial) | | 7.41-7.36 (m, 1H), 7.25-7.19 (m, 2H), 4.47 (br d, J = 14.0 |
| | | | Hz, 1H), 4.36 (br d, J = 10.1 Hz, 1H), 3.77 (br dd, J = 14.1, |
| | | | 6.3 Hz, 1H), 3.17-3.05 (m, 2H), 3.01-2.94 (m, 1H), 2.37- |
| | | | 2.32 (m, 1H), 1.86-1.79 (m, 2H), 1.57-1.53 (m, 1H) |
| 142 | Intermediate 69 | 1 | (DMSO-d₆) δ 13.48 (br s, 1H), 9.19 (br d, J = 8.8 Hz, 1H), |
| | | | 8.97 (dd, J = 4.1, 1.6 Hz, 1H), 8.48 (s, 1H), 8.32 (br d, J = 7.7 |
| | | | Hz, 1H), 8.02 (d, J = 7.7 Hz, 1H), 7.66 (br s, 3H), 7.58 (dd, |
| | | | J = 8.7, 4.1 Hz, 1H), 7.52-7.46 (m, 1H), 7.41-7.35 (m, 1H), |
| | | | 7.25-7.19 (m, 2H), 5.94-5.89 (m, 1H), 4.49 (br d, J = 14.0 |
| | | | Hz, 1H), 4.36 (br d, J = 9.4 Hz, 1H), 3.77 (br dd, J = 13.9, 6.1 |
| | | | Hz, 1H), 3.15-3.05 (m, 2H), 2.99-2.92 (m, 1H), 2.38-2.31 |
| | | | (m, 1H), 1.85-1.76 (m, 2H), 1.57-1.53 (m, 1H), 1.51 (d, |
| | | | J = 6.4 Hz, 3H) |
| 143 | imidazo[1,2-a]pyridin- | 1 | Method 2: m/z 455 (M + H)⁺, RT 1.08 min |
| | 5-ylboronic acid | | |
| 145 | Intermediate 53 | 1 | (DMSO-d₆) δ 13.21 (br s, 1H), 8.50 (s, 1H), 8.42 (s, 1H), |
| | | | 7.73 (br d, J = 8.8 Hz, 1H), 7.66 (br s, 3H), 7.51-7.46 (m, |
| | | | 2H), 7.40-7.36 (m, 1H), 7.25-7.19 (m, 2H), 4.46 (br d, |
| | | | J = 14.9 Hz, 1H), 4.34 (br d, J = 9.9 Hz, 1H), 4.19 (s, 3H), |
| | | | 3.70 (br dd, J = 13.2, 6.3 Hz, 1H), 3.18-3.05 (m, 2H), 2.96- |
| | | | 2.88 (m, 1H), 2.64 (s, 3H), 2.36-2.30 (m, 1H), 1.83-1.75 |
| | | | (m, 2H), 1.56-1.52 (m, 1H) |
| 146 | 8-methoxy-5-(4,4,5,5- | 1 | (DMSO-d₆) δ 13.42 (br s, 1H), 9.38 (br d, J = 8.8 Hz, 1H), |
| | tetramethyl-1,3,2- | | 8.96-8.94 (m, 1H), 8.47 (s, 1H), 8.39 (d, J = 8.1 Hz, 1H), |
| | dioxaborolan-2- | | 7.72-7.65 (m, 4H), 7.50-7.44 (m, 2H), 7.40-7.34 (m, 1H), |
| | yl)quinoline | | 7.24-7.19 (m, 2H), 4.46 (br d, J = 14.0 Hz, 1H), 4.35 (br d, |
| | (commercial) | | J = 10.3 Hz, 1H), 4.07 (s, 3H), 3.74 (br dd, J = 13.4, 6.5 Hz, |
| | | | 1H), 3.17-3.05 (m, 2H), 2.99-2.91 (m, 1H), 2.38-2.31 (m, |
| | | | 1H), 1.84-1.78 (m, 2H), 1.56-1.52 (m, 1H) |
| 147 | Intermediate 76 | 1 | (DMSO-d₆) δ 13.54 (s, 1H), 8.53 (s, 1H), 8.45 (d, J = 10.6 |
| | | | Hz, 2H), 7.99 (d, J = 2.2 Hz, 1H), 7.65 (br s, 3H), 7.49 (br t, |
| | | | J = 7.0 Hz, 1H), 7.41-7.35 (m, 1H), 7.32 (d, J = 2.0 Hz, 1H), |
| | | | 7.24-7.19 (m, 2H), 4.46 (br d, J = 14.0 Hz, 1H), 4.35 (br d, |
| | | | J = 9.2 Hz, 1H), 3.90 (s, 3H), 3.76 (br dd, J = 14.3, 6.6 Hz, |
| | | | 1H), 3.15-3.03 (m, 2H), 3.00-2.93 (m, 1H), 2.37-2.32 (m, |
| | | | 1H), 1.83-1.76 (m, 2H), 1.57-1.53 (m, 1H) |
| 159 | Intermediate 77 | 1 | (DMSO-d₆) δ 13.47 (br s, 1H), 9.23 (br d, J = 7.7 Hz, 1H), |
| | | | 8.95 (dd, J = 4.0, 1.8 Hz, 1H), 8.48 (s, 1H), 8.35 (d, J = 7.5 |
| | | | Hz, 1H), 7.97 (d, J = 7.5 Hz, 1H), 7.66 (br s, 3H), 7.60 (dd, |
| | | | J = 8.6, 4.2 Hz, 1H), 7.48 (br t, J = 6.9 Hz, 1H), 7.40-7.35 (m, |
| | | | 1H), 7.25-7.19 (m, 2H), 5.24 (s, 2H), 4.48 (br d, J = 14.3 Hz, |
| | | | 1H), 4.36 (br d, J = 9.9 Hz, 1H), 3.75 (br dd, J = 14.3, 6.1 Hz, |
| | | | 1H), 3.16-3.07 (m, 2H), 2.99-2.93 (m, 1H), 2.36-2.32 (m, |
| | | | 1H), 1.85-1.77 (m, 2H), 1.56-1.53 (m, 1H) |
| 160 | Intermediate 78 | 1 | (DMSO-d₆) δ 13.56 (br s, 1H), 9.31 (d, J = 9.0 Hz, 1H), 9.04 |
| | | | (dd, J = 4.2, 1.5 Hz, 1H), 8.49 (s, 1H), 8.37 (d, J = 8.1 Hz, |
| | | | 1H), 7.75-7.68 (m, 6H), 7.51-7.47 (m, 1H), 7.40-7.35 (m, |
| | | | 1H), 7.24-7.19 (m, 2H), 4.47 (br d, J = 14.3 Hz, 1H), 4.36 |
| | | | (br d, J = 9.9 Hz, 1H), 3.76 (br dd, J = 14.3, 6.4 Hz, 1H), 3.17- |
| | | | 2.94 (m, 3H), 2.38-2.32 (m, 1H), 1.85-1.77 (m, 2H), 1.57- |
| | | | 1.53 (m, 1H) |
| 161 | Intermediate 79 | 1 | (DMSO-d₆) δ 13.43 (s, 1H), 8.57 (d, J = 7.2 Hz, 1H), 8.50 |
| | | | (s, 1H), 8.13 (d, J = 2.0 Hz, 1H), 7.65 (br s, 3H), 7.50-7.45 |
| | | | (m, 2H), 7.40-7.35 (m, 1H), 7.24-7.18 (m, 2H), 7.01 (d, |
| | | | J = 7.2 Hz, 1H), 4.46 (br d, J = 14.0 Hz, 1H), 4.35 (br d, J = 9.7 |
| | | | Hz, 1H), 3.75 (br dd, J = 14.4, 6.5 Hz, 1H), 3.15-3.04 (m, |
| | | | 2H), 2.99-2.92 (m, 1H), 2.76 (s, 3H), 2.36-2.31 (m, 1H), |
| | | | 1.85-1.79 (m, 2H), 1.56-1.52 (m, 1H) |
| 162 | (3-bromo-2- | 1 | (DMSO-d₆) δ 13.44 (br s, 1H), 8.43 (s, 1H), 7.90 (d, J = 7.9 |
| | chlorophenyl)boronic | | Hz, 1H), 7.71-7.69 (m, 1H), 7.65 (br s, 3H), 7.48 (t, J = 7.7 |
| | acid (commercial) | | Hz, 1H), 7.44-7.35 (m, 2H), 7.25-7.19 (m, 2H), 4.44 (br d, |
| | | | J = 14.5 Hz, 1H), 4.33 (br d, J = 9.5 Hz, 1H), 3.71 (br dd, |
| | | | J = 14.2, 6.3 Hz, 1H), 3.18-3.06 (m, 2H), 2.97-2.89 (m, 1H), |
| | | | 2.36-2.31 (m, 1H), 1.82-1.77 (m, 2H), 1.56-1.52 (m, 1H) |
| 164 | Intermediate 80 | 1 | (DMSO-d₆ + TFA) 10.18 (dd, J = 8.8, 1.3 Hz, 1H), 9.25-9.23 |
| | | | (m, 1H), 8.76 (d, J = 8.3 Hz, 1H), 8.5 (s, 1H), 8.23 (dd, |
| | | | J = 8.8, 5.3 Hz, 1H), 7.86 (d, J = 8.6 Hz, 1H), 7.69 (br s, 3H), |
| | | | 7.50-7.47 (m, 1H), 7.39-7.33 (m, 1H), 7.22-7.17 (m, 2H), |
| | | | 4.54-4.42 (m, 3H), 4.28 (br d, J = 2.9 Hz, 1H), 3.77 (br dd, |
| | | | J = 14.3, 6.4 Hz, 1H), 3.19-2.94 (m, 3H), 2.39-2.31 (m, 1H), |
| | | | 1.85-1.78 (m, 2H), 1.59-1.53 (m, 4H) |
| 165 | Intermediate 81 | 1 | (DMSO-d₆ + TFA) 9.61-9.58 (m, 1H), 9.11 (br d, J = 4.2 Hz, |
| | | | 1H), 8.51-8.49 (m, 2H), 8.06 (br d, J = 7.7 Hz, 1H), 7.86- |
| | | | 7.83 (m, 1H), 7.69 (br s, 3H), 7.49 (br t, J = 7.1 Hz, 1H), |
| | | | 7.39-7.33 (m, 1H), 7.22-7.17 (m, 2H), 5.53 (q, J = 6.2 Hz, |
| | | | 1H), 4.49-4.45 (m, 1H), 4.37-4.34 (m, 1H), 3.75 (br dd, |
| | | | J = 14.4, 6.3 Hz, 1H), 3.30 (s, 3H), 3.18-3.05 (m, 2H), 3.00- |
| | | | 2.92 (m, 1H), 2.38-2.32 (m, 1H), 1.85-1.76 (m, 2H), 1.57- |
| | | | 1.53 (m, 4H) |
| 166 | (8-methoxyquinolin-4- | 1 | (DMSO-d₆ + TFA) 9.25 (d, J = 5.7 Hz, 1H), 9.17-9.13 (m, |
| | yl)boronic acid | | 2H), 8.63 (s, 1H), 7.96 (t, J = 8.3 Hz, 1H), 7.74-7.69 (m, 4H), |
| | | | 7.49 (br t, J = 7.6 Hz, 1H), 7.37-7.34 (m, 1H), 7.22-7.17 (m, |
| | | | 2H), 4.44 (br d, J = 13.6 Hz, 1H), 4.36 (br d, J = 11.7 Hz, 1H), |
| | | | 4.20 (s, 3H), 3.82 (br dd, J = 14.3, 6.6 Hz, 1H), 3.17-2.99 |
| | | | (m, 3H), 2.40-2.32 (m, 1H), 1.85-1.78 (m, 2H), 1.59-1.53 |
| | | | (m, 1H) |
| 167 | Intermediate 82 | 1 | (DMSO-d₆) δ 13.62 (br s, 1H), 9.14 (s, 1H), 9.11 (s, 1H), |
| | | | 8.48 (s, 1H), 8.26-8.20 (m, 2H), 7.67 (br s, 3H), 7.49 (br t, |
| | | | J = 7.7 Hz, 1H), 7.41-7.37 (m, 1H), 7.25-7.19 (m, 2H), 4.47 |
| | | | (br d, J = 13.8 Hz, 1H), 4.35 (br d, J = 10.5 Hz, 1H), 3.74 (br |
| | | | dd, J = 14.1, 6.5 Hz, 1H), 3.19-3.06 (m, 2H), 2.99-2.91 (m, |
| | | | 1H), 2.39-2.30 (m, 1H), 1.85-1.78 (m, 2H), 1.59-1.53 (m, |
| | | | 1H) |
| 171 | Intermediate 83 | 1 | (DMSO-d₆) δ 13.42 (br s, 1H), 8.95 (br d, J = 4.0 Hz, 1H), |
| | | | 8.52 (s, 1H), 8.49-8.46 (m, 2H), 8.02 (s, 1H), 7.66 (br s, |
| | | | 3H), 7.58 (br dd, J = 7.5, 4.0 Hz, 1H), 7.49 (br t, J = 7.7 Hz, |
| | | | 1H), 7.41-7.35 (m, 1H), 7.25-7.19 (m, 2H), 4.46 (br d, |
| | | | J = 14.5 Hz, 1H), 4.35 (br d, J = 11.0 Hz, 1H), 3.74 (br dd, |
| | | | J = 14.5, 6.4 Hz, 1H), 3.19-3.05 (m, 2H), 2.99-2.91 (m, 1H), |
| | | | 2.73 (s, 3H), 2.38-2.30 (m, 1H), 1.85-1.76 (m, 2H), 1.57- |
| | | | 1.53 (m, 1H) |
| 172 | Intermediate 84 | 1 | (DMSO-d₆) δ 13.44 (br s, 1H), 9.00 (br d, J = 4.2 Hz, 1H), |
| | | | 8.74 (br d, J = 8.1 Hz, 1H), 8.45 (s, 1H), 8.13 (br d, J = 8.8 |
| | | | Hz, 1H), 8.01 (br d, J = 8.8 Hz, 1H), 7.71-7.63 (m, 4H), 7.49 |
| | | | (br t, J = 7.5 Hz, 1H), 7.41-7.35 (m, 1H), 7.25-7.19 (m, 2H), |
| | | | 4.46 (br d, J = 14.7 Hz, 1H), 4.35 (br d, J = 9.2 Hz, 1H), 3.73 |
| | | | (br dd, J = 14.3, 6.6 Hz, 1H), 3.18-3.05 (m, 2H), 2.99-2.91 |
| | | | (m, 1H), 2.81 (s, 3H), 2.39-2.30 (m, 1H), 1.84-1.75 (m, |
| | | | 2H), 1.57-1.53 (m, 1H) |
| 173 | Intermediate 85 | 1 | (DMSO-d₆) δ 13.39 (br s, 1H), 8.94 (br d, J = 3.1 Hz, 1H), |
| | | | 8.83 (s, 1H), 8.56 (br d, J = 4.8 Hz, 2H), 8.46 (br d, J = 7.7 |
| | | | Hz, 1H), 7.67 (br s, 3H), 7.59 (br dd, J = 8.3, 4.2 Hz, 1H), |
| | | | 7.49 (br t, J = 7.5 Hz, 1H), 7.41-7.36 (m, 1H), 7.25-7.19 (m, |
| | | | 2H), 4.47 (br d, J = 14.5 Hz, 1H), 4.36 (br d, J = 11.6 Hz, 1H), |
| | | | 3.77 (br dd, J = 14.4, 6.0 Hz, 1H), 3.17-3.05 (m, 2H), 3.00- |
| | | | 2.94 (m, 1H), 2.81 (s, 3H), 2.39-2.32 (m, 1H), 1.84-1.75 |
| | | | (m, 2H), 1.57-1.53 (m, 1H) |
| 174 | Intermediate 86 | 1 | (DMSO-d₆) δ 13.50 (br s, 1H), 8.98-8.95 (br m, 1H), 8.82 |
| | | | (s, 1H), 8.59-8.54 (br m, 2H), 8.44 (br d, J = 12.1 Hz, 1H), |
| | | | 7.71-7.62 (br m, 4H), 7.49 (br t, J = 7.3 Hz, 1H), 7.41-7.35 |
| | | | (m, 1H), 7.25-7.19 (m, 2H), 4.46 (br d, J = 14.0 Hz, 1H), |
| | | | 4.36 (br d, J = 12.1 Hz, 1H), 3.77 (br dd, J = 14.3, 6.1 Hz, |
| | | | 1H), 3.17-2.95 (m, 3H), 2.38-2.31 (m, 1H), 1.85-1.74 (m, |
| | | | 2H), 1.58-1.52 (m, 1H) |
| 175 | Intermediate 87 | 1 | (DMSO-d₆) δ 13.54 (br s, 1H), 8.97 (br d, J = 3.7 Hz, 1H), |
| | | | 8.78 (d, J = 7.7 Hz, 1H), 8.54-8.52 (m, 2H), 7.93 (d, J = 11.8 |
| | | | Hz, 1H), 7.67 (br s, 3H), 7.60 (dd, J = 8.3, 4.4 Hz, 1H), 7.49 |
| | | | (br t, J = 7.6 Hz, 1H), 7.41-7.35 (m, 1H), 7.25-7.19 (m, 2H), |
| | | | 4.47 (br d, J = 14.0 Hz, 1H), 4.36 (br d, J = 9.9 Hz, 1H), 3.75 |
| | | | (br dd, J = 14.3, 6.1 Hz, 1H), 3.17-3.04 (m, 2H), 3.01-2.93 |
| | | | (m, 1H), 2.38-2.31 (m, 1H), 1.85-1.74 (m, 2H), 1.57-1.53 |
| | | | (m, 1H) |
| 176 | 3-chloropyridin-4- | 1 | (DMSO-d₆) δ 13.69 (br s, 1H), 8.79 (s, 1H), 8.66 (d, J = 5.0 |
| | yl)boronic acid | | Hz, 1H), 8.49 (s, 1H), 8.03 (d, J = 5.0 Hz, 1H), 7.65 (br s, |
| | | | 3H), 7.48 (br t, J = 7.9 Hz, 1H), 7.41-7.35 (m, 1H), 7.24-7.18 |
| | | | (m, 2H), 4.44 (br d, J = 14.0 Hz, 1H), 4.34 (br d, J = 9.9 Hz, |
| | | | 1H), 3.74 (br dd, J = 14.3, 6.6 Hz, 1H), 3.15-3.04 (m, 2H), |
| | | | 2.98-2.91 (m, 1H), 2.39-2.30 (m, 1H), 1.85-1.74 (m, 2H), |
| | | | 1.57-1.51 (m, 1H); |
| 186 | 4-(4,4,5,5-tetramethyl- | 1 | (DMSO-d₆) δ 13.74 (s, 1H), 8.57 (s, 1H), 8.53 (d, J = 5.3 Hz, |
| | 1,3,2-dioxaborolan-2- | | 1H), 8.45 (d, J = 5.0 Hz, 1H), 8.20-8.19 (m, 1H), 7.66 (br s, |
| | yl)furo[2,3-b]pyridine | | 3H), 7.60 (br d, J = 2.2 Hz, 1H), 7.49 (br t, J = 7.6 Hz, 1H), |
| | | | 7.41-7.35 (m, 1H), 7.25-7.19 (m, 2H), 4.47 (br d, J = 14.0 |
| | | | Hz, 1H), 4.36 (br d, J = 9.7 Hz, 1H), 3.77 (br dd, J = 14.4, 6.3 |
| | | | Hz, 1H), 3.16-2.94 (m, 3H), 2.39-2.32 (m, 1H), 1.84-1.76 |
| | | | (m, 2H), 1.57-1.53 (m, 1H) |
| 187 | 5-(4,4,5,5-tetramethyl- | 2 | (DMSO-d₆) δ 13.10 (br s, 1H), 10.52 (s, 1H), 8.44 (s, 1H), |
| | 1,3,2-dioxaborolan-2- | | 8.23-8.20 (m, 2H), 7.65 (br s, 3H), 7.49 (br t, J = 7.5 Hz, |
| | yl)indolin-2-one | | 1H), 7.40-7.36 (m, 1H), 7.25-7.19 (m, 2H), 6.94 (d, J = 7.9 |
| | | | Hz, 1H), 4.44 (br d, J = 14.3 Hz, 1H), 4.33 (br d, J = 9.4 Hz, |
| | | | 1H), 3.73 (br dd, J = 14.1, 6.3 Hz, 1H), 3.59 (s, 2H), 3.17- |
| | | | 2.91 (m, 3H), 2.36-2.31 (m, 1H), 1.84-1.76 (m, 2H), 1.56- |
| | | | 1.52 (m, 1H) |
| 188 | 2-methoxy-7-(4,4,5,5- | 1 | (DMSO-d₆) δ 13.62 (s, 1H), 9.11 (s, 1H), 8.74 (s, 1H), 8.71 |
| | tetramethyl-1,3,2- | | (s, 1H), 8.66 (d, J = 8.6 Hz, 1H), 8.25 (d, J = 8.8 Hz, 1H), 7.79 |
| | dioxaborolan-2- | | (br s, 3H), 7.63 (br t, J = 7.7 Hz, 1H), 7.52-7.50 (m, 1H), |
| | yl)quinoxaline | | 7.39-7.33 (m, 2H), 4.59 (br d, J = 14.5 Hz, 1H), 4.50 (br d, |
| | | | J = 10.3 Hz, 1H), 4.23 (s, 3H), 3.91 (br dd, J = 14.6, 6.7 Hz, |
| | | | 1H), 3.29-3.08 (m, 3H), 2.51-2.47 (m, 1H), 1.98-1.89 (m, |
| | | | 2H), 1.72-1.68 (m, 1H) |
| 189 | 3-chloro-2- | 1 | (DMSO-d₆ + TFA) 8.77 (d, J = 5.9 Hz, 1H), 8.53 (s, 1H), 8.35 |
| | methylpyridin-4- | | (d, J = 5.7 Hz, 1H), 7.69 (br s, 3H), 7.49 (br t, J = 7.5 Hz, 1H), |
| | yl)boronic acid | | 7.39-7.34 (m, 1H), 7.22-7.17 (m, 2H), 4.43 (br d, J = 14.5 |
| | | | Hz, 1H), 4.33 (br d, J = 9.4 Hz, 1H), 3.77 (br dd, J = 14.4, 6.5 |
| | | | Hz, 1H), 3.16-2.93 (m, 3H), 2.78 (s, 3H), 2.38-2.31 (m, |
| | | | 1H), 1.83-1.76 (m, 2H), 1.57-1.53 (m, 1H) |
| 190 | 3-chloro-2- | 1 | (DMSO-d₆ + TFA) 8.47 (s, 1H), 8.25 (d, J = 4.8 Hz, 1H), 7.67 |
| | (dimethylamino)pyridin- | | (br s, 3H), 7.50-7.46 (m, 1H), 7.42 (d, J = 5.1 Hz, 1H), 7.37- |
| | 4-yl)boronic acid | | 7.33 (m, 1H), 7.21-7.16 (m, 2H), 4.44 (br d, J = 14.3 Hz, |
| | | | 1H), 4.33 (br d, J = 9.7 Hz, 1H), 3.72 (br dd, J = 14.1, 6.3 Hz, |
| | | | 1H), 3.15-3.09 (m, 2H), 3.06 (s, 6H), 2.98-2.92 (m, 1H), |
| | | | 2.35-2.31 (m, 1H), 1.83-1.77 (m, 2H), 1.56-1.52 (m, 1H) |
| 191 | 7-methoxy-4-(4,4,5,5- | 1 | (DMSO-d₆ + TFA) 9.48 (d, J = 8.1 Hz, 1H), 9.30 (d, J = 6.7 |
| | tetramethyl-1,3,2- | | Hz, 1H), 8.90 (d, J = 6.3 Hz, 1H), 8.64 (s, 1H), 7.72-7.64 (m, |
| | dioxaborolan-2- | | 5H), 7.52-7.48 (m, 1H), 7.39-7.35 (m, 1H), 7.23-7.18 (m, |
| | yl)quinoline | | 2H), 4.44 (br d, J = 14.7 Hz, 1H), 4.36 (br d, J = 10.2 Hz, 1H), |
| | (commercial) | | 4.05 (s, 3H), 3.83 (br dd, J = 14.0, 6.1 Hz, 1H), 3.16-3.01 |
| | | | (m, 3H), 2.39-2.34 (m, 1H), 1.86-1.79 (m, 2H), 1.59-1.55 |
| | | | (m, 1H) |
| 192 | N,N-dimethyl-1-(5- | 1 | (MeOH-d₄) δ 9.27 (br d, J = 8.3 Hz, 1H), 9.07 (d, J = 3.1 Hz, |
| | (4,4,5,5-tetramethyl- | | 1H), 8.44 (s, 1H), 8.37 (d, J = 7.5 Hz, 1H), 8.04 (d, J = 7.5 |
| | 1,3,2-dioxaborolan-2- | | Hz, 1H), 7.71 (dd, J = 8.8, 4.2 Hz, 1H), 7.49 (br t, J = 7.2 Hz, |
| | yl)quinolin-8- | | 1H), 7.43-7.40 (m, 1H), 7.26-7.18 (m, 2H), 4.94 (s, 2H), |
| | yl)methanamine | | 4.63 (br d, J = 14.0 Hz, 1H), 4.47 (br d, J = 8.3 Hz, 1H), 3.85 |
| | | | (br dd, J = 14.3, 6.4 Hz, 1H), 3.42-3.34 (m, 2H), 3.13-3.07 |
| | | | (m, 1H), 3.00 (s, 6H), 2.54-2.48 (m, 1H), 2.03-1.90 (m, |
| | | | 2H), 1.73-1.69 (m, 1H) |
| 193 | 4-methyl-5-(4,4,5,5- | 1 | (DMSO-d₆) δ 13.68 (s, 1H), 8.51 (s, 1H), 8.30 (d, J = 9.4 Hz, |
| | tetramethyl-1,3,2- | | 1H), 8.00 (d, J = 9.2 Hz, 1H), 7.67 (br s, 3H), 7.49 (br t, |
| | dioxaborolan-2- | | J = 7.2 Hz, 1H), 7.40-7.35 (m, 1H), 7.24-7.19 (m, 2H), 4.45 |
| | yl)benzo[c][1,2,5] | | (br d, J = 14.0 Hz, 1H), 4.34 (br d, J = 9.9 Hz, 1H), 3.75 (br |
| | oxadiazole | | dd, J = 14.3, 6.6 Hz, 1H), 3.16-3.04 (m, 2H), 3.00-2.93 (m, |
| | | | 1H), 2.87 (s, 3H), 2.39-2.31 (m, 1H), 1.85-1.76 (m, 2H), |
| | | | 1.57-1.53 (m, 1H) |
| 194 | Intermediate 182 | 1 | (DMSO-d₆) δ 13.18 (br s, 1H), 8.43 (s, 1H), 8.39 (s, 1H), |
| | | | 7.66 (br s, 3H), 7.51-7.35 (m, 4H), 7.25-7.19 (m, 2H), 4.45 |
| | | | (br d, J = 14.7 Hz, 1H), 4.33 (br d, J = 16.2 Hz, 1H), 4.18 (s, |
| | | | 3H), 3.70 (br dd, J = 14.7, 8.1 Hz, 1H), 3.19-3.06 (m, 2H), |
| | | | 2.96-2.87 (m, 1H), 2.58-2.52 (m, 2H), 2.38-2.29 (m, 1H), |
| | | | 1.84-1.75 (m, 2H), 1.58-1.52 (m, 1H), 0.77-0.72 (m, 3H) |
| 195 | 3-fluoro-2- | 1 | (DMSO-d₆) δ 13.69 (br s, 1H), 8.52 (s, 1H), 8.42 (d, J = 4.8 |
| | methylpyridin-4- | | Hz, 1H), 8.11 (t, J = 5.2 Hz, 1H), 7.66 (br s, 3H), 7.49 (br t, |
| | yl)boronic acid | | J = 7.1 Hz, 1H), 7.41-7.36 (m, 1H), 7.25-7.19 (m, 2H), 4.45 |
| | | | (br d, J = 14.0 Hz, 1H), 4.34 (br d, J = 9.7 Hz, 1H), 3.75 (br |
| | | | dd, J = 14.3, 6.6 Hz, 1H), 3.14-2.93 (m, 3H), 2.56-2.55 (m, |
| | | | 3H), 2.37-2.32 (m, 1H), 1.83-1.76 (m, 2H), 1.57-1.53 (m, |
| | | | 1H) |
| 196 | 2-amino-5- | 1 | (DMSO-d₆) δ 13.53 (br s, 1H), 8.45 (s, 1H), 8.07 (s, 1H), |
| | chloropyridin-4- | | 7.65 (br s, 3H), 7.48 (br t, J = 7.8 Hz, 1H), 7.41-7.34 (m, |
| | yl)boronic acid | | 1H), 7.24-7.19 (m, 2H), 6.99 (s, 1H), 6.41 (br s, 2H), 4.43 |
| | | | (br d, J = 13.8 Hz, 1H), 4.33 (br d, J = 9.9 Hz, 1H), 3.73 (br |
| | | | dd, J = 14.4, 6.5 Hz, 1H), 3.17-3.01 (m, 2H), 2.98-2.90 (m, |
| | | | 1H), 2.38-2.31 (m, 1H), 1.82-1.75 (m, 2H), 1.56-1.52 (m, |
| | | | 1H) |
| 197 | Intermediate 183 | 1 | (DMSO-d₆) δ 13.54 (br s, 1H), 8.45 (s, 1H), 8.14 (br d, |
| | | | J = 5.7 Hz, 1H), 7.65 (br s, 3H), 7.50-7.46 (m, 1H), 7.41- |
| | | | 7.37 (m, 1H), 7.24-7.19 (m, 2H), 7.10-7.05 (m, 1H), 6.89 |
| | | | (br s, 1H), 4.44 (br d, J = 14.0 Hz, 1H), 4.32 (br d, J = 10.1 |
| | | | Hz, 1H), 3.75-3.69 (m, 1H), 3.19-3.02 (m, 2H), 2.99-2.89 |
| | | | (m, 1H), 2.83-2.77 (m, 1H), 2.37-2.29 (m, 1H), 1.85-1.74 |
| | | | (m, 2H), 1.58-1.51 (m, 1H), 0.78-0.70 (m, 2H), 0.63-0.56 |
| | | | (m, 2H) |
| 198 | 1-methyl-6-oxo-1,6- | 1 | (DMSO-d₆) δ 13.67 (br s, 1H), 8.49 (s, 1H), 7.67 (br s, 3H), |
| | dihydropyridin-2- | | 7.54-7.40 (m, 2H), 7.40-7.36 (m, 1H), 7.24-7.18 (m, 2H), |
| | yl)boronic acid | | 6.63 (d, J = 5.9 Hz, 1H), 6.52 (d, J = 9.0 Hz, 1H), 4.43 (br d, |
| | | | J = 14.3 Hz, 1H), 4.33 (br d, J = 10.1 Hz, 1H), 3.77-3.71 (m, |
| | | | 1H), 3.54 (s, 3H), 3.15-2.91 (m, 3H), 2.36-2.30 (m, 1H), |
| | | | 1.83-1.75 (m, 2H), 1.56-1.52 (m, 1H) |
| 201 | 8-methoxy-2-methyl- | 1 | (DMSO-d₆) δ 13.43 (br s, 1H), 9.36 (br s, 1H), 8.47 (s, 1H), |
| | 5-(4,4,5,5-tetramethyl- | | 8.37 (br d, J = 7.9 Hz, 1H), 7.68 (br s, 4H), 7.51-7.47 (m, |
| | 1,3,2-dioxaborolan-2- | | 2H), 7.41-7.35 (m, 1H), 7.25-7.19 (m, 2H), 4.46 (br d, |
| | yl)quinoline | | J = 14.3 Hz, 1H), 4.35 (br d, J = 9.2 Hz, 1H), 4.09 (s, 3H), |
| | | | 3.75 (br dd, J = 14.0, 6.5 Hz, 1H), 3.16-3.05 (m, 2H), 2.98- |
| | | | 2.93 (m, 1H), 2.78 (s, 3H), 2.37-2.32 (m, 1H), 1.84-1.77 |
| | | | (m, 2H), 1.57-1.53 (m, 1H) |
| 202 | Intermediate 155 | 1 | (DMSO-d₆) δ 13.48 (br s, 1H), 8.59 (s, 1H), 8.49 (s, 1H), |
| | | | 7.99 (d, J = 9.0 Hz, 1H), 7.90 (d, J = 9.0 Hz, 1H), 7.66 (br s, |
| | | | 4H), 7.51-7.46 (m, 1H), 7.40-7.35 (m, 1H), 7.24-7.18 (m, |
| | | | 2H), 4.46 (br d, J = 14.5 Hz, 1H), 4.34 (br d, J = 9.9 Hz, 1H), |
| | | | 4.25 (s, 3H), 3.74 (br dd, J = 14.1, 6.6 Hz, 1H), 3.16-3.04 |
| | | | (m, 2H), 2.98-2.91 (m, 1H), 2.38-2.31 (m, 1H), 1.83-1.76 |
| | | | (m, 2H), 1.56-1.52 (m, 1H) |
| 203 | Intermediate 45 | 1 | (DMSO-d₆) δ 13.37 (br s, 1H), 8.56 (s, 1H), 8.43 (s, 1H), |
| | | | 7.70-7.63 (m, 5H), 7.48 (br t, J = 7.7 Hz, 1H), 7.40-7.36 (m, |
| | | | 1H), 7.24-7.19 (m, 2H), 4.45 (br d, J = 14.0 Hz, 1H), 4.34 |
| | | | (br d, J = 9.8 Hz, 1H), 4.23 (s, 3H), 3.74-3.70 (m, 1H), 3.18- |
| | | | 3.04 (m, 2H), 2.97-2.90 (m, 1H), 2.38-2.31 (m, 1H), 1.82- |
| | | | 1.76 (m, 2H), 1.56-1.52 (m, 1H) |
| 204 | Intermediate 156 | 1 | (DMSO-d₆) δ 13.36 (br s, 1H), 8.46 (s, 1H), 8.11 (d, J = 8.6 |
| | | | Hz, 1H), 7.77 (d, J = 8.6 Hz, 1H), 7.66 (br s, 3H), 7.49 (br t, |
| | | | J = 7.7 Hz, 1H), 7.41-7.35 (m, 1H), 7.25-7.19 (m, 2H), 4.47 |
| | | | (br d, J = 13.8 Hz, 1H), 4.39-4.35 (br m, 1H), 4.34 (s, 3H), |
| | | | 3.75-3.70 (m, 1H), 3.18-3.05 (m, 2H), 2.97-2.93 (m, 1H), |
| | | | 2.90 (s, 3H), 2.38-2.32 (m, 1H), 1.83-1.77 (m, 2H), 1.57- |
| | | | 1.53 (m, 1H) |
| 205 | Intermediate 157 | 1 | (DMSO-d₆) δ 13.37 (br s, 1H), 8.45 (s, 1H), 7.98 (d, J = 8.8 |
| | | | Hz, 1H), 7.82 (d, J = 8.8 Hz, 1H), 7.68 (br s, 3H), 7.49 (br t, |
| | | | J = 7.7 Hz, 1H), 7.40-7.35 (m, 1H), 7.24-7.18 (m, 2H), 4.53 |
| | | | (s, 3H), 4.46 (br d, J = 14.3 Hz, 1H), 4.34 (br d, J = 10.1 Hz, |
| | | | 1H), 3.73 (br dd, J = 14.5, 6.4 Hz, 1H), 3.17-3.04 (m, 2H), |
| | | | 2.96-2.91 (m, 1H), 2.79 (s, 3H), 2.37-2.31 (m, 1H), 1.82- |
| | | | 1.77 (m, 2H), 1.56-1.52 (m, 1H) |
| 206 | 3,3-dimethyl-5- | 1 | (DMSO-d₆) δ 13.13 (br s, 1H), 10.50 (s, 1H), 8.47 (s, 1H), |
| | (4,4,5,5-tetramethyl- | | 8.25 (dd, J = 8.1, 1.5 Hz, 1H), 8.17 (s, 1H), 7.66 (br s, 3H), |
| | 1,3,2-dioxaborolan-2- | | 7.48 (br t, J = 7.1 Hz, 1H), 7.41-7.36 (m, 1H), 7.25-7.19 (m, |
| | yl)indolin-2-one | | 2H), 6.98 (d, J = 8.1 Hz, 1H), 4.45 (br d, J = 14.5 Hz, 1H), |
| | | | 4.34 (br d, J = 9.0 Hz, 1H), 3.75-3.70 (m, 1H), 3.19-3.02 (m, |
| | | | 2H), 2.98-2.90 (m, 1H), 2.38-2.31 (m, 1H), 1.84-1.77 (m, |
| | | | 2H), 1.55-1.51 (m, 1H), 1.32 (s, 6H) |
| 207 | 5-chloro-6-(4,4,5,5- | 1 | (DMSO-d₆) δ 13.32 (br s, 1H), 10.51 (s, 1H), 8.40 (s, 1H), |
| | tetramethyl-1,3,2- | | 7.66 (br s, 3H), 7.48 (br t, J = 7.7 Hz, 1H), 7.40-7.35 (m, |
| | dioxaborolan-2-yl)- | | 1H), 7.31 (d, J = 8.3 Hz, 1H), 7.24-7.18 (m, 2H), 7.11 (d, |
| | 2H- | | J = 8.3 Hz, 1H), 4.68 (s, 2H), 4.43 (br d, J = 14.0 Hz, 1H), |
| | benzo[b][1,4]oxazin- | | 4.32 (br d, J = 10.5 Hz, 1H), 3.70 (dd, J = 14.3, 6.4 Hz, 1H), |
| | 3(4H)-one | | 3.16-3.05 (m, 2H), 2.93-2.88 (m, 1H), 2.36-2.30 (m, 1H), |
| | | | 1.82-1.75 (m, 2H), 1.56-1.51 (m, 1H) |
| 208 | Intermediate 158 | 1 | (DMSO-d₆ + TFA) 8.59 (s, 1H), 8.42 (s, 1H), 7.60-7.63 (m, |
| | | | 5H), 7.48 (br t, J = 7.1 Hz, 1H), 7.39-7.34 (m, 1H), 7.23-7.18 |
| | | | (m, 2H), 4.55-4.45 (m, 3H), 4.33 (br d, J = 9.7 Hz, 1H), 3.71 |
| | | | (dd, J = 14.0, 6.2 Hz, 1H), 3.18-3.05 (m, 2H), 2.96-2.90 (m, |
| | | | 1H), 2.36-2.30 (m, 1H), 1.82-1.77 (m, 2H), 1.56-1.53 (m, |
| | | | 4H) |
| 209 | 5-(4,4,5,5-tetramethyl- | 1 | (DMSO-d₆) δ 13.45 (s, 1H), 11.38 (br d, J = 4.8 Hz, 1H), |
| | 1,3,2-dioxaborolan-2- | | 8.46 (s, 1H), 8.38 (br d, J = 8.1 Hz, 1H), 8.30 (d, J = 8.1 Hz, |
| | yl)isoquinolin-1(2H)- | | 1H), 7.71-7.60 (m, 4H), 7.48 (br t, J = 7.1 Hz, 1H), 7.41-7.35 |
| | one | | (m, 1H), 7.25-7.19 (m, 3H), 4.47 (br d, J = 14.3 Hz, 1H), |
| | | | 4.35 (br d, J = 9.0 Hz, 1H), 3.73 (dd, J = 14.1, 6.0 Hz, 1H), |
| | | | 3.17-3.03 (m, 2H), 2.99-2.90 (m, 1H), 2.37-2.32 (m, 1H), |
| | | | 1.83-1.75 (m, 2H), 1.56-1.53 (m, 1H) |
| 210 | 5-(4,4,5,5-tetramethyl- | 1 | (DMSO-d₆) δ 13.50 (s, 1H), 11.88 (s, 1H), 8.67 (d, J = 9.9 |
| | 1,3,2-dioxaborolan-2- | | Hz, 1H), 8.48 (s, 1H), 7.87 (br d, J = 7.0 Hz, 1H), 7.65-7.62 |
| | yl)quinolin-2(1H)-one | | (m, 4H), 7.49 (br t, J = 7.5 Hz, 1H), 7.40-7.36 (m, 2H), 7.25- |
| | | | 7.19 (m, 2H), 6.55 (dd, J = 10.1, 2.0 Hz, 1H), 4.47 (br d, |
| | | | J = 13.8 Hz, 1H), 4.35 (br d, J = 9.4 Hz, 1H), 3.74 (dd, |
| | | | J = 14.4, 6.3 Hz, 1H), 3.17-3.04 (m, 2H), 2.98-2.92 (m, 1H), |
| | | | 2.39-2.31 (m, 1H), 1.83-1.77 (m, 2H), 1.57-1.53 (m, 1H) |
| 211 | Intermediate 159 | 1 | Method 2: m/z 470 (M + H)⁺, RT 1.10 min. |
| 212 | N,N-dimethyl-5- | 1 | (DMSO-d₆) δ 13.58 (br s, 1H), 9.31 (d, J = 8.6 Hz, 1H), 8.99 |
| | (4,4,5,5-tetramethyl- | | (br s, 1H), 8.51 (s, 1H), 8.42 (br d, J = 7.5 Hz, 1H), 7.81 (d, |
| | 1,3,2-dioxaborolan-2- | | J = 7.5 Hz, 1H), 7.68-7.63 (m, 4H), 7.49 (brt, J = 7.7 Hz, 1H), |
| | yl)quinoline-8- | | 7.41-7.35 (m, 1H), 7.25-7.19 (m, 2H), 4.48 (br d, J = 13.6 |
| | carboxamide | | Hz, 1H), 4.36 (br d, J = 12.3 Hz, 1H), 3.75 (dd, J = 14.0, 6.1 |
| | | | Hz, 1H), 3.12 (s, 3H), 3.11-3.04 (m, 2H), 3.00-2.94 (m, |
| | | | 1H), 2.73 (s, 3H), 2.40-2.34 (m, 1H), 1.81-1.75 (m, 2H), |
| | | | 1.57-1.53 (m, 1H) |
| 214 | 6-(bis(tert- | 1 | (DMSO-d₆) δ 13.79 (br s, 1H), 8.53 (s, 1H), 7.81 (br d, |
| | butoxycarbonyl)amino)- | | J = 8.1 Hz, 1H), 7.69 (br s, 3H), 7.58 (br s, 1H), 7.48 (br t, |
| | 5-methoxypyridin-2- | | J = 7.1 Hz, 1H), 7.41-7.35 (m, 1H), 7.24-7.18 (m, 2H), 4.39 |
| | yl)boronic acid | | (br d, J = 14.5 Hz, 1H), 4.32 (br d, J = 13.2 Hz, 1H), 3.97 (s, |
| | (prepared as reported | | 3H), 3.78 (dd, J = 14.1, 6.7 Hz, 1H), 3.19-2.94 (m, 3H), |
| | for Intermediate 17) | | 2.39-2.32 (m, 1H), 1.83-1.75 (m, 2H), 1.56-1.52 (m, 1H) |
| 215 | Intermediate 160 | 1 | (DMSO-d₆) δ 13.43 (br s, 1H), 8.43 (s, 1H), 7.71 (d, J = 2.2 |
| | | | Hz, 1H), 7.67-7.63 (br m, 4H), 7.48 (br t, J = 7.6 Hz, 1H), |
| | | | 7.41-7.36 (m, 1H), 7.27-7.19 (m, 3H), 5.99 (d, J = 2.4 Hz, |
| | | | 1H), 4.44 (br d, J = 14.5 Hz, 1H), 4.33 (br d, J = 11.4 Hz, 1H), |
| | | | 3.76 (s, 3H), 3.75-3.70 (m, 1H), 3.17-3.05 (m, 2H), 2.97- |
| | | | 2.90 (m, 1H), 2.37-2.31 (m, 1H), 1.82-1.77 (m, 2H), 1.56- |
| | | | 1.53 (m, 1H) |
| 216 | 4-chloro-5-(4,4,5,5- | 1 | (DMSO-d₆) δ 13.27 (br s, 1H), 10.73 (s, 1H), 8.39 (s, 1H), |
| | tetramethyl-1,3,2- | | 7.66 (br s, 3H), 7.59 (br d, J = 8.1 Hz, 1H), 7.48 (br t, J = 7.3 |
| | dioxaborolan-2- | | Hz, 1H), 7.40-7.35 (m, 1H), 7.24-7.18 (m, 2H), 6.91 (br d, |
| | yl)indolin-2-one | | J = 7.9 Hz, 1H), 4.43 (br d, J = 13.8 Hz, 1H), 4.32 (br d, |
| | | | J = 11.9 Hz, 1H), 3.72-3.66 (m, 1H), 3.60 (m, 2H), 3.16-3.04 |
| | | | (m, 2H), 2.95-2.89 (m, 1H), 2.36-2.30 (m, 1H), 1.83-1.75 |
| | | | (m, 2H), 1.55-1.51 (m, 1H) |
| 217 | 1-methyl-5-(4,4,5,5- | 1 | (DMSO-d₆) δ 13.11 (br s, 1H), 8.45 (s, 1H), 8.31 (br d, |
| | tetramethyl-1,3,2- | | J = 8.3 Hz, 1H), 8.24 (s, 1H), 7.67 (br s, 3H), 7.48 (brt, J = 7.1 |
| | dioxaborolan-2- | | Hz, 1H), 7.41-7.35 (m, 1H), 7.24-7.19 (m, 2H), 7.11 (br d, |
| | yl)indolin-2-one | | J = 8.3 Hz, 1H), 4.43 (br d, J = 14.3 Hz, 1H), 4.32 (br d, |
| | | | J = 10.7 Hz, 1H), 3.73 (dd, J = 14.3, 6.4 Hz, 1H), 3.66 (s, 2H), |
| | | | 3.17 (s, 3H), 3.12-3.04 (m, 2H), 2.97-2.91 (m, 1H), 2.37- |
| | | | 2.31 (m, 1H), 1.83-1.75 (m, 2H), 1.56-1.52 (m, 1H) |
| 218 | 5-(4,4,5,5-tetramethyl- | 2 | (DMSO-d₆) δ 13.41 (br s, 1H), 8.63-8.46 (br m, 4H), 7.79 |
| | 1,3,2-dioxaborolan-2- | | (br d, J = 7.0 Hz, 1H), 7.66 (br s, 3H), 7.52-7.45 (m, 1H), |
| | yl)isoindolin-1-one | | 7.42-7.35 (m, 1H), 7.25-7.18 (m, 2H), 4.50-4.29 (brm, 4H), |
| | (commercial) | | 3.78-3.72 (brm, 1H), 3.18-2.91 (m, 3H), 2.40-2.32 (m, 1H), |
| | | | 1.85-1.76 (m, 2H), 1.59-1.53 (m, 1H) |
| 219 | 8-methoxy-5-(4,4,5,5- | 1 | (DMSO-d₆) δ 13.42 (s, 1H), 9.63 (s, 1H), 8.81 (br s, 1H), |
| | tetramethyl-1,3,2- | | 8.62-8.60 (m, 2H), 8.47 (s, 1H), 7.65 (br s, 3H), 7.50-7.47 |
| | dioxaborolan-2- | | (m, 1H), 7.39-7.34 (m, 2H), 7.24-7.19 (m, 2H), 4.47 (br d, |
| | yl)isoquinoline | | J = 14.0 Hz, 1H), 4.35 (br d, J = 10.2 Hz, 1H), 4.11 (s, 3H), |
| | | | 3.74 (dd, J = 14.4, 6.3 Hz, 1H), 3.21-3.04 (m, 2H), 2.98-2.91 |
| | | | (m, 1H), 2.38-2.31 (m, 1H), 1.83-1.76 (m, 2H), 1.56-1.53 |
| | | | (m, 1H) |
| 220 | Intermediate 161 | 1 | (DMSO-d₆) δ 13.67 (br s, 1H), 10.21 (br d, J = 4.0 Hz, 1H), |
| | | | 9.43 (br d, J = 8.6 Hz, 1H), 9.10 (dd, J = 4.1, 1.6 Hz, 1H), 8.70 |
| | | | (d, J = 7.7 Hz, 1H), 8.53 (s, 1H), 8.48 (d, J = 7.7 Hz, 1H), 7.98 |
| | | | (br d, J = 3.3 Hz, 1H), 7.74 (dd, J = 8.8, 4.2 Hz, 1H), 7.67 (br |
| | | | s, 3H), 7.49 (br t, J = 7.6 Hz, 1H), 7.41-7.35 (m, 1H), 7.25- |
| | | | 7.19 (m, 2H), 4.49 (br d, J = 14.3 Hz, 1H), 4.37 (br d, J = 9.4 |
| | | | Hz, 1H), 3.77 (dd, J = 14.4, 6.5 Hz, 1H), 3.17-3.04 (m, 2H), |
| | | | 3.01-2.95 (m, 1H), 2.39-2.33 (m, 1H), 1.85-1.78 (m, 2H), |
| | | | 1.58-1.54 (m, 1H) |
| 222 | Intermediate 162 | 1 | (DMSO-d₆) δ 13.53 (br s, 1H), 9.04 (d, J = 1.5 Hz, 1H), 9.00 |
| | | | (d, J = 1.8 Hz, 1H), 8.49 (s, 1H), 8.34 (d, J = 9.0 Hz, 1H), 8.07 |
| | | | (d, J = 8.8 Hz, 1H), 7.68 (br s, 3H), 7.49 (br t, J = 7.5 Hz, 1H), |
| | | | 7.41-7.36 (m, 1H), 7.25-7.19 (m, 2H), 4.48 (br d, J = 14.3 |
| | | | Hz, 1H), 4.36 (br d, J = 10.5 Hz, 1H), 3.77-3.72 (m, 1H), |
| | | | 3.23-3.04 (m, 2H), 2.96 (s, 3H), 2.95-2.90 (m, 1H), 2.39- |
| | | | 2.32 (m, 1H), 1.83-1.77 (m, 2H), 1.57-1.53 (m, 1H) |
| 223 | 2-chloro-4- | 1 | (MeOH-d₄) δ 8.36 (s, 1H), 8.18 (d, J = 1.5 Hz, 1H), 8.06 (dd, |
| | (methoxycarbonyl) | | J = 8.1, 1.5 Hz, 1H), 7.83 (d, J = 8.1 Hz, 1H), 7.46 (brt, J = 7.1 |
| | phenyl)boronic acid | | Hz, 1H), 7.40-7.35 (m, 1H), 7.24-7.15 (m, 2H), 4.63 (br d, |
| | | | J = 14.5 Hz, 1H), 4.44 (br d, J = 10.7 Hz, 1H), 3.97 (s, 3H), |
| | | | 3.78 (dd, J = 14.5, 6.1 Hz, 1H), 3.37-3.27 (m, 2H), 3.08-3.01 |
| | | | (m, 1H), 2.51-2.45 (m, 1H), 1.99-1.90 (m, 2H), 1.69-1.65 |
| | | | (m, 1H) |
| 224 | imidazo[1,2-a]pyridin- | 2 | (DMSO-d₆) δ 13.54 (s, 1H), 9.66-9.64 (m, 1H), 8.65 (s, |
| | 3-ylboronic acid | | 1H), 8.53 (s, 1H), 7.90-7.87 (m, 1H), 7.66 (br s, 4H), 7.50- |
| | | | 7.47 (m, 1H), 7.41-7.37 (m, 2H), 7.25-7.19 (m, 2H), 4.46 |
| | | | (br d, J = 14.0 Hz, 1H), 4.37 (br d, J = 10.2 Hz, 1H), 3.79 (br |
| | | | dd, J = 13.8, 6.4 Hz, 1H), 3.18-3.13 (m, 1H), 3.10-3.04 (m, |
| | | | 1H), 3.01-2.97 (m, 1H), 2.39-2.32 (m, 1H), 1.85-1.78 (m, |
| | | | 2H), 1.57-1.53 (m, 1H) |
| 225 | 7-fluoro-8-methoxy-5- | 1 | (DMSO-d₆) δ 13.59 (br s, 1H), 9.42 (d, J = 7.5 Hz, 1H), 9.01 |
| | (4,4,5,5-tetramethyl- | | (dd, J = 4.1, 1.4 Hz, 1H), 8.52 (s, 1H), 8.40 (d, J = 11.8 Hz, |
| | 1,3,2-dioxaborolan-2- | | 1H), 7.72-7.63 (m, 4H), 7.49 (br t, J = 7.2 Hz, 1H), 7.40-7.36 |
| | yl)quinoline | | (m, 1H), 7.24-7.19 (m, 2H), 4.47 (br d, J = 14.2 Hz, 1H), |
| | | | 4.35 (br d, J = 9.0 Hz, 1H), 4.18 (s, 3H), 3.76 (br dd, J = 14.3 |
| | | | Hz, 6.6 Hz, 1H), 3.17-2.94 (m, 3H), 2.39-2.32 (m, 1H), |
| | | | 1.85-1.76 (m, 2H), 1.57-1.53 (m, 1H) |
| 226 | 8-fluoro-2-methyl-5- | 1 | (DMSO-d₆) δ 13.50 (br s, 1H), 9.18 (br d, J = 8.8 Hz, 1H), |
| | (4,4,5,5-tetramethyl- | | 8.48 (s, 1H), 8.30 (dd, J = 8.1, 5.0 Hz, 1H), 7.74-7.68 (m, |
| | 1,3,2-dioxaborolan-2- | | 4H), 7.59 (d, J = 9.0 Hz, 1H), 7.49 (brt, J = 7.7 Hz, 1H), 7.41- |
| | yl)quinoline | | 7.35 (m, 1H), 7.25-7.19 (m, 2H), 4.47 (br d, J = 14.3 Hz, |
| | | | 1H), 4.35 (br d, J = 11.4 Hz, 1H), 3.76 (br dd, J = 14.1, 6.3 |
| | | | Hz, 1H), 3.17-3.05 (m, 2H), 3.00-2.93 (m, 1H), 2.73 (s, |
| | | | 3H), 2.39-2.32 (m, 1H), 1.84-1.76 (m, 2H), 1.57-1.53 (m, |
| | | | 1H) |
| 227 | Intermediate 163 | 1 | (MeOD-d₄) 8.36 (s, 1H), 7.82 (d, J = 8.1 Hz, 1H), 7.66 (d, |
| | | | J = 8.1 Hz, 1H), 7.48-7.36 (m, 2H), 7.24-7.15 (m, 2H), 4.63 |
| | | | (br d, J = 14.7 Hz, 1H), 4.44 (br d, J = 11.2 Hz, 1H), 3.78 (br |
| | | | dd, J = 14.5, 6.1 Hz, 1H), 3.37-3.27 (m, 2H), 3.08-3.01 (m, |
| | | | 1H), 2.51-2.44 (m, 1H), 1.99-1.87 (m, 2H), 1.69-1.67 (m, |
| | | | 1H) |
| 228 | 5-chloro-4-methyl-6- | 1 | 228 (MeOD-d₄) δ 8.34 (s, 1H), 7.55-7.28 (m, 3H), 7.26- |
| | (4,4,5,5-tetramethyl- | | 7.09 (m, 3H), 4.66-4.56 (m, 3H), 4.49-4.38 (m, 1H), 3.83- |
| | 1,3,2-dioxaborolan-2- | | 3.75 (m, 1H), 3.57 (s, 3H), 3.39-3.31 (m, 1H), 3.10-2.99 |
| | yl)-2H- | | (m, 1H), 2.53-2.38 (m, 1H), 2.05-1.84 (m, 2H), 1.73-1.59 |
| | benzo[b][1,4]oxazin- | | (m, 1H), 1.39-1.22 (m, 1H) |
| | 3(4H)-one | | |
| 229 | Intermediate 178 | 1 | (DMSO-d₆) δ 13.41 (s, 1H), 8.87 (s, 1H), 8.44 (s, 1H), 7.84- |
| | | | 7.70 (m, 2H), 7.66 (br s, 3H), 7.53-7.45 (m, 1H), 7.42-7.35 |
| | | | (m, 1H), 7.27-7.17 (m, 2H), 5.65-5.52 (m, 1H), 4.65 (br d, |
| | | | J = 4.4 Hz, 4H), 4.46 (br d, J = 14.0 Hz, 1H), 4.40-4.27 (m, |
| | | | 1H), 3.84-3.62 (m, 1H), 3.16-2.97 (m, 3H), 2.37-2.28 (m, |
| | | | 1H), 2.00 (br dd, J = 6.4, 7.5 Hz, 1H), 1.92-1.72 (m, 1H), |
| | | | 1.64-1.51 (m, 1H) |
| 230 | 7-(4,4,5,5-tetramethyl- | 1 | (DMSO-d₆) δ 13.21 (s, 1H), 10.26 (s, 1H), 8.46 (s, 1H), |
| | 1,3,2-dioxaborolan-2- | | 8.00 (br d, J = 7.7 Hz, 1H), 7.82 (s, 1H), 7.67 (br s, 3H), |
| | yl)-3,4- | | 7.52-7.48 (m, 1H), 7.42-7.37 (m, 1H), 7.31-7.21 (m, 3H), |
| | dihydroquinolin- | | 4.47 (br d, J = 13.4 Hz, 1H), 4.35 (br d, J = 11.8 Hz, 1H), 3.74 |
| | 2(1H)-one | | (br dd, J = 14.1, 6.2 Hz, 1H), 3.20-3.03 (m, 2H), 2.95-2.91 |
| | | | (m, 3H), 2.50-2.48 (m, 2H), 2.39-2.33 (m, 1H), 1.84-1.77 |
| | | | (m, 2H), 1.58-1.54 (m, 1H) |
| 231 | 7-(4,4,5,5-tetramethyl- | 1 | (DMSO-d₆) δ 13.39 (s, 1H), 11.88 (s, 1H), 8.49 (s, 1H), |
| | 1,3,2-dioxaborolan-2- | | 8.29 (s, 1H), 8.21 (d, J = 7.9 Hz, 1H), 7.92 (d, J = 9.7 Hz, 1H), |
| | yl)quinolin-2(1H)-one | | 7.76 (d, J = 8.3 Hz, 1H), 7.65 (br s, 3H), 7.48 (t, J = 7.6 Hz, |
| | | | 1H), 7.41-7.35 (m, 1H), 7.25-7.19 (m, 2H), 6.50 (dd, J = 9.7, |
| | | | 1.8 Hz, 1H), 4.46 (br d, J = 14.3 Hz, 1H), 4.36 (br d, J = 10.5 |
| | | | Hz, 1H), 3.75 (br dd, J = 14.5, 6.6 Hz, 1H), 3.18-3.03 (m, |
| | | | 2H), 2.99-2.92 (m, 1H), 2.39-2.32 (m, 1H), 1.84-1.75 (m, |
| | | | 2H), 1.57-1.53 (m, 1H) |
| 232 | 1,3-dimethyl-5- | 1 | (DMSO-d₆) δ 13.15 (s, 1H), 8.49 (s, 1H), 8.19 (d, J = 8.8 Hz, |
| | (4,4,5,5-tetramethyl- | | 1H), 8.02 (s, 1H), 7.65 (br s, 3H), 7.50-7.47 (m, 1H), 7.42- |
| | 1,3,2-dioxaborolan-2- | | 7.36 (m, 1H), 7.28-7.19 (m, 3H), 4.47 (br d, J = 14.1 Hz, |
| | yl)-1,3-dihydro-2H- | | 1H), 4.34 (br d, J = 10.3 Hz, 1H), 3.74 (br dd, J = 14.2, 6.1 |
| | benzo[d]imidazol-2- | | Hz, 1H), 3.40 (s, 6H), 3.17-3.05 (m, 2H), 2.98-2.90 (m, |
| | one (commercial) | | 1H), 2.39-2.31 (m, 1H), 1.85-1.76 (m, 2H), 1.57-1.51 (m, |
| | | | 1H) |
| 233 | 6-(4,4,5,5-tetramethyl- | 1 | (DMSO-d₆) δ 13.20 (br s, 1H), 10.52 (s, 1H), 8.45 (s, 1H), |
| | 1,3,2-dioxaborolan-2- | | 7.98 (d, J = 8.1 Hz, 1H), 7.84 (s, 1H), 7.65 (br s, 3H), 7.50- |
| | yl)indolin-2-one | | 7.46 (m, 1H), 7.40-7.35 (m, 1H), 7.31 (d, J = 7.9 Hz, 1H), |
| | (commercial) | | 7.25-7.19 (m, 2H), 4.45 (br d, J = 13.4 Hz, 1H), 4.34 (br d, |
| | | | J = 10.2 Hz, 1H), 3.73 (br dd, J = 14.3, 6.1 Hz, 1H), 3.53 (s, |
| | | | 2H), 3.17-3.03 (m, 2H), 2.97-2.90 (m, 1H), 2.37-2.30 (m, |
| | | | 1H), 1.83-1.77 (m, 2H), 1.56-1.52 (m, 1H) |
| 234 | 6-(4,4,5,5-tetramethyl- | 1 | (DMSO-d₆) δ 13.23 (s, 1H), 11.87 (s, 1H), 8.60 (s, 1H), |
| | 1,3,2-dioxaborolan-2- | | 8.48 (s, 1H), 8.43 (d, J = 8.8 Hz, 1H), 8.01 (d, J = 9.7 Hz, 1H), |
| | yl)quinolin-2(1H)-one | | 7.62 (br s, 3H), 7.48 (br t, J = 7.5 Hz, 1H), 7.43-7.35 (m, |
| | | | 2H), 7.24-7.18 (m, 2H), 6.54 (d, J = 9.9 Hz, 1H), 4.45 (br d, |
| | | | J = 14.5 Hz, 1H), 4.34 (br d, J = 9.9 Hz, 1H), 3.74 (br dd, |
| | | | J = 14.1, 6.5 Hz, 1H), 3.15-3.03 (m, 2H), 2.98-2.91 (m, 1H), |
| | | | 2.38-2.31 (m, 1H), 1.82-1.75 (m, 2H), 1.55-1.52 (m, 1H) |
| 235 | 1-oxo-1,2- | 1 | (DMSO-d₆) δ 13.65 (s, 1H), 10.76 (s, 1H), 8.55 (s, 1H), |
| | dihydroisoquinolin-3- | | 8.24 (d, J = 7.9 Hz, 1H), 7.80-7.73 (m, 2H), 7.65 (br s, 3H), |
| | yl)boronic acid | | 7.55-7.46 (m, 2H), 7.41-7.35 (m, 1H), 7.25-7.19 (m, 2H), |
| | | | 4.44 (br d, J = 14.7 Hz, 1H), 4.34 (br d, J = 14.0 Hz, 1H), 3.78 |
| | | | (br dd, J = 14.3, 6.8 Hz, 1H), 3.17-2.93 (m, 3H), 2.39-2.31 |
| | | | (m, 1H), 1.83-1.75 (m, 2H), 1.57-1.53 (m, 1H) |
| 236 | 8-fluoro-5-(4,4,5,5- | 1 | (DMSO-d₆) δ 13.49 (s, 1H), 11.86 (br s, 1H), 8.70 (br d, |
| | tetramethyl-1,3,2- | | J = 9.9 Hz, 1H), 8.47 (s, 1H), 7.89-7.86 (m, 1H), 7.65 (br s, |
| | dioxaborolan-2- | | 3H), 7.61-7.66 (m, 1H), 7.48 (br d, J = 7.6 Hz, 1H), 7.41- |
| | yl)quinolin-2(1H)-one | | 7.35 (m, 1H), 7.24-7.19 (m, 2H), 6.63 (br d, J = 9.9 Hz, 1H), |
| | | | 4.46 (br d, J = 14.5 Hz, 1H), 4.34 (br d, J = 11.6 Hz, 1H), 3.74 |
| | | | (br dd, J = 14.4, 6.5 Hz, 1H), 3.18-3.02 (m, 2H), 2.98-2.92 |
| | | | (m, 1H), 2.38-2.31 (m, 1H), 1.84-1.75 (m, 2H), 1.56-1.52 |
| | | | (m, 1H) |
| 237 | 8-chloro-7-(4,4,5,5- | 1 | (DMSO-d₆) δ 13.50 (br s, 1H), 10.96 (br s, 1H), 8.44 (s, |
| | tetramethyl-1,3,2- | | 1H), 8.04 (br d, J = 9.9 Hz, 1H), 7.79 (br d, J = 8.1 Hz, 1H), |
| | dioxaborolan-2- | | 7.66 (br s, 3H), 7.59 (d, J = 8.1 Hz, 1H), 7.48 (br d, J = 7.7 |
| | yl)quinolin-2(1H)-one | | Hz, 1H), 7.40-7.35 (m, 1H), 7.24-7.19 (m, 2H), 6.66 (br d, |
| | | | J = 8.8 Hz, 1H), 4.45 (br d, J = 13.8 Hz, 1H), 4.33 (br d, |
| | | | J = 10.8 Hz, 1H), 3.73 (br dd, J = 13.9, 6.1 Hz, 1H), 3.19-3.04 |
| | | | (m, 2H), 2.98-2.89 (m, 1H), 2.36-2.30 (m, 1H), 1.82-1.75 |
| | | | (m, 2H), 1.56-1.52 (m, 1H) |
| 238 | 6-(4,4,5,5-tetramethyl- | 1 | DMSO-d₆) δ 10.21 (s, 1H), 9.11 (s, 1H), 8.47 (s, 1H), 8.14 |
| | 1,3,2-dioxaborolan-2- | | (s, 1H), 8.11 (d, J = 8.1 Hz, 1H), 7.79 (br s, 3H), 7.53 (t, |
| | yl)-3,4- | | J = 7.0 Hz, 1H), 7.40-7.34 (m, 1H), 7.27-7.18 (m, 2H), 6.96 |
| | dihydroquinolin- | | (br d, J = 8.3 Hz, 1H), 4.49 (br d, J = 15.1 Hz, 1H), 4.33 (br |
| | 2(1H)-one | | d, J = 10.3 Hz, 1H), 3.75-3.70 (m, 1H), 3.17-3.04 (m, 2H), |
| | | | 2.98-2.90 (m, 3H), 2.39-2.30 (m, 1H), 1.84-1.72 (m, 4H), |
| | | | 1.55-1.52 (m, 1H) |
| 239 | 5-(4,4,5,5-tetramethyl- | 1 | (DMSO-d₆) δ 13.06 (br s, 1H), 10.73 (s, 2H), 8.45 (s, 1H), |
| | 1,3,2-dioxaborolan-2- | | 8.00-7.98 (m, 2H), 7.73 (br s, 2H), 7.70 (s, 1H), 7.51 (br t, |
| | yl)-1,3-dihydro-2H- | | J = 7.1 Hz, 1H), 7.40-7.35 (m, 1H), 7.24-7.18 (m, 2H), 7.03 |
| | benzo[d]imidazol-2- | | (br d, J = 8.1 Hz, 1H), 4.48 (br d, J = 13.4 Hz, 1H), 4.33 (br |
| | one | | d, J = 9.4 Hz, 1H), 3.72 (br dd, J = 14.5, 6.6 Hz, 1H), 3.18- |
| | | | 3.03 (m, 2H), 2.96-2.88 (m, 1H), 2.38-2.30 (m, 1H), 1.84- |
| | | | 1.75 (m, 2H), 1.56-1.52 (m, 1H) |
| 240 | 6-(4,4,5,5-tetramethyl- | 1 | (DMSO-d₆) δ 13.47 (br s, 1H), 9.07 (s, 1H), 8.60 (s, 1H), |
| | 1,3,2-dioxaborolan-2- | | 8.54 (s, 1H), 8.43 (d, J = 8.6 Hz, 1H), 8.29 (d, J = 8.6 Hz, 1H), |
| | yl)-2-((2- | | 7.69-7.65 (m, 4H), 7.51-7.47 (m, 1H), 7.41-7.35 (m, 1H), |
| | (trimethylsilyl)ethoxy) | | 7.24-7.19 (m, 2H), 6.63 (d, J = 7.5 Hz, 1H), 4.48 (br d, |
| | | | J = 13.2 Hz, 1H), 4.36 (br d, J = 10.1 Hz, 1H), 3.76 (br dd, |
| | methyl)isoquinolin- | | J = 14.2, 6.1 Hz, 1H), 3.18-3.02 (m, 2H), 2.99-2.90 (m, 1H), |
| | 1(2H)-one | | 2.40-2.32 (m, 1H), 1.85-1.76 (m, 2H), 1.58-1.52 (m, 1H) |
| 241 | 2,3-dichloro-4- | 1 | (DMSO-d₆) δ 13.45 (br s, 1H), 8.45 (s, 1H), 8.05 (s, 1H), |
| | (4,4,5,5-tetramethyl- | | 7.80-7.68 (m, 5H), 7.53-7.50 (m, 2H), 7.40-7.35 (m, 1H), |
| | 1,3,2-dioxaborolan-2- | | 7.24-7.18 (m, 2H), 4.47 (br d, J = 14.5 Hz, 1H), 4.33 (br d, |
| | yl)benzamide | | J = 11.4 Hz, 1H), 3.72 (br dd, J = 14.4, 6.5 Hz, 1H), 3.18-3.03 |
| | (prepared as reported | | (m, 2H), 2.96-2.89 (m, 1H), 2.37-2.29 (m, 1H), 1.85-1.76 |
| | for Intermediate 161) | | (m, 2H), 1.56-1.52 (m, 1H) |
| 242 | quinolin-5-ylboronic | 1 | (DMSO-d₆) δ 13.56 (br s, 1H), 9.32 (br d, J = 7.9 Hz, 1H), |
| | acid (commercial) | | 9.02 (d, J = 2.6 Hz, 1H), 8.49 (s, 1H), 8.41 (d, J = 7.0 Hz, 1H), |
| | | | 8.13 (d, J = 8.6 Hz, 1H), 7.96 (t, J = 7.9 Hz, 1H), 7.72-7.64 |
| | | | (m, 4H), 7.49 (br t, J = 7.0 Hz, 1H), 7.41-7.35 (m, 1H), 7.25- |
| | | | 7.19 (m, 2H), 4.47 (br d, J = 14.0 Hz, 1H), 4.36 (br d, J = 10.1 |
| | | | Hz, 1H), 3.76 (br dd, J = 14.0, 6.4 Hz, 1H), 3.18-3.04 (m, |
| | | | 2H), 3.00-2.94 (m, 1H), 2.38-2.31 (m, 1H), 1.85-1.77 (m, |
| | | | 2H), 1.57-1.53 (m, 1H) |
| 243 | 4-chloro-5-(4,4,5,5- | 1 | (DMSO-d₆) δ 13.53 (br s, 1H), 13.36 (br s, 1H), 8.42 (s, |
| | tetramethyl-1,3,2- | | 1H), 8.21 (s, 1H), 7.74 (br d, J = 8.3 Hz, 1H), 7.66-7.64 (m, |
| | dioxaborolan-2-yl)- | | 4H), 7.49 (t, J = 7.7 Hz, 1H), 7.41-7.36 (m, 1H), 7.24-7.19 |
| | 2H-indazole | | (m, 2H), 4.46 (br d, J = 14.9 Hz, 1H), 4.34 (br d, J = 9.7 Hz, |
| | (commercial) | | 1H), 3.71 (br dd, J = 13.8, 6.4 Hz, 1H), 3.19-3.03 (m, 2H), |
| | | | 2.97-2.91 (m, 1H), 2.38-2.31 (m, 1H), 1.82-1.75 (m, 2H), |
| | | | 1.56-1.52 (m, 1H) |
| 244 | 2-methyl-5-(4,4,5,5- | 1 | (DMSO-d₆) δ 13.40 (br s, 1H), 8.52-8.49 (m, 3H), 7.79 (d, |
| | tetramethyl-1,3,2- | | J = 7.7 Hz, 1H), 7.65 (br s, 3H), 7.48 (br t, J = 7.3 Hz, 1H), |
| | dioxaborolan-2- | | 7.41-7.35 (m, 1H), 7.25-7.19 (m, 2H), 4.56 (s, 2H), 4.43 (br |
| | yl)isoindolin-1-one | | d, J = 13.8 Hz, 1H), 4.34 (br d, J = 11.6 Hz, 1H), 3.76 (br dd, |
| | (commercial) | | J = 14.1, 6.3 Hz, 1H), 3.18-3.05 (m, 2H), 3.10 (s, 3H), 2.99- |
| | | | 2.91 (m, 1H), 2.40-2.31 (m, 1H), 1.84-1.75 (m, 2H), 1.57- |
| | | | 1.53 (m, 1H) |
| 245 | 3-chloro-4-(4,4,5,5- | 2 | (DMSO-d₆) δ 13.61 (br s, 1H), 8.46 (s, 1H), 8.22 (s, 1H), |
| | tetramethyl-1,3,2- | | 8.07 (d, J = 8.1 Hz, 1H), 7.97 (d, J = 8.6 Hz, 1H), 7.64 (br s, |
| | dioxaborolan-2- | | 3H), 7.48 (br t, J = 7.6 Hz, 1H), 7.40-7.35 (m, 1H), 7.24-7.19 |
| | yl)benzonitrile | | (m, 2H), 4.43 (br d, J = 14.0 Hz, 1H), 4.33 (br d, J = 12.9 Hz, |
| | (commercial) | | 1H), 3.73 (br dd, J = 14.6, 6.3 Hz, 1H), 3.18-3.02 (m, 2H), |
| | | | 2.98-2.91 (m, 1H), 2.38-2.31 (m, 1H), 1.84-1.75 (m, 2H), |
| | | | 1.56-1.52 (m, 1H) |
| 246 | 2-methyl-4-(4,4,5,5- | 1 | (DMSO-d₆) δ 13.29 (br s, 1H), 8.50 (s, 1H), 8.21-8.16 (m, |
| | tetramethyl-1,3,2- | | 2H), 7.74 (s, 1H), 7.65 (br s, 3H), 7.51-7.46 (m, 2H), 7.41- |
| | dioxaborolan-2- | | 7.35 (m, 2H), 7.24-7.19 (m, 2H), 4.45 (br d, J = 14.0 Hz, |
| | yl)benzamide | | 1H), 4.34 (br d, J = 10.7 Hz, 1H), 3.73 (br dd, J = 14.4, 6.0 |
| | | | Hz, 1H), 3.19-3.03 (m, 2H), 2.98-2.91 (m, 1H), 2.46 (s, |
| | | | 3H), 2.36-2.31 (m, 1H), 1.83-1.75 (m, 2H), 1.56-1.52 (m, |
| | | | 1H) |
| 247 | isoquinolin-6- | 2 | (DMSO-d₆) δ 13.66 (br s, 1H), 9.59 (s, 1H), 9.10 (s, 1H), |
| | ylboronic acid | | 8.76 (br d, J = 8.6 Hz, 1H), 8.62-8.59 (m, 2H), 8.43 (br d, |
| | (commercial) | | J = 8.8 Hz, 1H), 8.25 (br d, J = 5.9 Hz, 1H), 7.72 (br s, 3H), |
| | | | 7.51 (br t, J = 7.5 Hz, 1H), 7.40-7.36 (m, 1H), 7.25-7.19 (m, |
| | | | 2H), 4.47 (br d, J = 14.3 Hz, 1H), 4.36 (br d, J = 9.7 Hz, 1H), |
| | | | 3.79 (br dd, J = 13.8, 6.1 Hz, 1H), 3.18-3.04 (m, 2H), 3.01- |
| | | | 2.95 (m, 1H), 2.40-2.33 (m, 1H), 1.85-1.77 (m, 2H), 1.58- |
| | | | 1.54 (m, 1H) |
| 248 | isoquinolin-7- | 2 | (DMSO-d₆) δ 13.51 (br s, 1H), 9.61 (br s, 1H), 9.21 (s, 1H), |
| | ylboronic acid | | 8.82 (br d, J = 8.3 Hz, 1H), 8.59-8.56 (m, 2H), 8.21 (br d, |
| | (commercial) | | J = 8.8 Hz, 1H), 8.08 (br d, J = 5.9 Hz, 1H), 7.68 (br s, 3H), |
| | | | 7.49 (br t, J = 7.8 Hz, 1H), 7.41-7.36 (m, 1H), 7.25-7.19 (m, |
| | | | 2H), 4.45 (br d, J = 14.7 Hz, 1H), 4.36 (br d, J = 9.7 Hz, 1H), |
| | | | 3.79 (br dd, J = 14.3, 6.4 Hz, 1H), 3.19-3.04 (m, 2H), 3.02- |
| | | | 2.95 (m, 1H), 2.40-2.33 (m, 1H), 1.84-1.78 (m, 2H), 1.58- |
| | | | 1.54 (m, 1H) |
| 249 | (1,1-dioxido-3-oxo- | 1 | (DMSO-d₆) δ 13.47 (s, 1H), 9.07 (br s, 1H), 8.65 (s, 1H), |
| | 2,3- | | 8.55 (s, 1H), 7.75 (br d, J = 7.9 Hz, 1H), 7.69 (br s, 4H), |
| | dihydrobenzo[d]iso- | | 7.53-7.47 (m, 1H), 7.41-7.35 (m, 1H), 7.24-7.20 (m, 2H), |
| | thiazol-6-yl)boronic | | 4.49 (br d, J = 14.3 Hz, 1H), 4.35 (br d, J = 10.1 Hz, 1H), 3.75 |
| | acid | | (br dd, J = 14.1, 6.1 Hz, 1H), 3.16-3.02 (m, 2H), 2.99-2.92 |
| | | | (m, 1H), 2.40-2.33 (m, 1H), 1.85-1.76 (m, 2H), 1.58-1.53 |
| | | | (m, 1H) |
| 250 | 3-methyl-4-(4,4,5,5- | 1 | (DMSO-d₆) δ 13.37 (br s, 1H), 8.46 (s, 1H), 8.05-8.00 (m, |
| | tetramethyl-1,3,2- | | 2H), 7.86 (s, 1H), 7.81 (br d, J = 8.1 Hz, 1H), 7.73 (br s, 3H), |
| | dioxaborolan-2- | | 7.51 (br t, J = 7.9 Hz, 1H), 7.41-7.33 (m, 2H), 7.24-7.20 (m, |
| | yl)benzamide | | 2H), 4.48 (br d, J = 14.0 Hz, 1H), 4.34 (br d, J = 9.4 Hz, 1H), |
| | | | 3.75-3.70 (m, 1H), 3.18-3.03 (m, 2H), 2.97-2.89 (m, 1H), |
| | | | 2.57 (s, 3H), 2.39-2.31 (m, 1H), 1.85-1.74 (m, 2H), 1.56- |
| | | | 1.52 (m, 1H) |
| 251 | 7-(4,4,5,5-tetramethyl- | 1 | (DMSO-d₆) δ 13.27 (s, 1H), 8.89 (s, 1H), 8.50 (s, 1H), 8.40- |
| | 1,3,2-dioxaborolan-2- | | 8.38 (m, 1H), 7.99 (br s, 1H), 7.64 (br s, 3H), 7.52-7.34 (m, |
| | yl)-3,4- | | 3H), 7.25-7.19 (m, 2H), 4.46 (br d, J = 14.1 Hz, 1H), 4.35 |
| | dihydroisoquinolin- | | (br d, J = 9.3 Hz, 1H), 3.77-3.72 (m, 1H), 3.46-3.41 (m, 2H), |
| | 1(2H)-one | | 3.18-3.03 (m, 3H), 2.97-2.94 (m, 2H), 2.39-2.30 (m, 1H), |
| | | | 1.86-1.77 (m, 2H), 1.56-1.52 (m, 1H) |
| 252 | 7-(4,4,5,5-tetramethyl- | 1 | (DMSO-d₆) δ 13.46 (br s, 1H), 9.16 (s, 1H), 8.98 (br d, |
| | 1,3,2-dioxaborolan-2- | | J = 3.3 Hz, 1H), 8.57 (s, 1H), 8.53 (br d, J = 9.0 Hz, 1H), 8.47 |
| | yl)quinoline | | (br d, J = 8.3 Hz, 1H), 8.13 (d, J = 8.6 Hz, 1H), 7.66 (br s, |
| | (commercial) | | 3H), 7.62-7.59 (m, 1H), 7.49 (br t, J = 7.5 Hz, 1H), 7.41-7.35 |
| | | | (m, 1H), 7.25-7.19 (m, 2H), 4.48 (br d, J = 13.4 Hz, 1H), |
| | | | 4.38 (br d, J = 9.0 Hz, 1H), 3.79-3.74 (m, 1H), 3.18-3.05 (m, |
| | | | 2H), 3.01-2.93 (m, 1H), 2.40-2.33 (m, 1H), 1.85-1.77 (m, |
| | | | 2H), 1.58-1.54 (m, 1H) |
| 253 | 4-carbamoyl-5-chloro- | 1 | (DMSO-d₆) δ 13.56 (br s, 1H), 8.52 (s, 1H), 8.37 (d, J = 6.4 |
| | 2- | | Hz, 1H), 8.01 (s, 1H), 7.76 (br s, 1H), 7.66 (br s, 3H), 7.52- |
| | fluorophenyl)boronic | | 7.46 (m, 2H), 7.40-7.35 (m, 1H), 7.24-7.18 (m, 2H), 4.45 |
| | acid | | (br d, J = 13.6 Hz, 1H), 4.33 (br d, J = 13.4 Hz, 1H), 3.74 (br |
| | | | dd, J = 14.4, 6.7 Hz, 1H), 3.17-3.02 (m, 2H), 2.99-2.93 (m, |
| | | | 1H), 2.38-2.31 (m, 1H), 1.83-1.76 (m, 2H), 1.56-1.52 (m, |
| | | | 1H) |
| 254 | 7-(4,4,5,5-tetramethyl- | 1 | (DMSO-d₆) δ 13.53 (s, 1H), 12.70 (s, 1H), 9.30 (s, 1H), |
| | 1,3,2-dioxaborolan-2- | | 8.78 (dd, J = 8.2, 1.6 Hz, 1H), 8.58 (s, 1H), 8.40 (s, 1H), 8.04 |
| | yl)phthalazin-1(2H)- | | (d, J = 8.3 Hz, 1H), 7.66 (br s, 3H), 7.48 (br t, J = 7.7 Hz, 1H), |
| | one | | 7.41-7.35 (m, 1H), 7.25-7.19 (m, 2H), 4.46 (br d, J = 14.9 |
| | | | Hz, 1H), 4.36 (br d, J = 9.9 Hz, 1H), 3.77 (br dd, J = 14.3, 6.4 |
| | | | Hz, 1H), 3.20-3.04 (m, 2H), 3.01-2.94 (m, 1H), 2.40-2.32 |
| | | | (m, 1H), 1.84-1.77 (m, 2H), 1.57-1.53 (m, 1H) |
| 255 | 3-chloro-7-(4,4,5,5- | 1 | (DMSO-d₆) δ 13.48 (br s, 1H), 9.15 (s, 1H), 8.92 (d, J = 2.6 |
| | tetramethyl-1,3,2- | | Hz, 1H), 8.61 (d, J = 2.0 Hz, 1H), 8.57 (s, 1H), 8.55 (d, J = 9.5 |
| | dioxaborolan-2- | | Hz, 1H), 8.09 (d, J = 8.8 Hz, 1H), 7.67 (br s, 3H), 7.49 (d, |
| | yl)quinoline | | J = 7.6 Hz, 1H), 7.41-7.35 (m, 1H), 7.25-7.19 (m, 2H), 4.47 |
| | | | (br d, J = 14.5 Hz, 1H), 4.37 (br d, J = 9.7 Hz, 1H), 3.76 (br |
| | | | dd, J = 13.9, 6.1 Hz, 1H), 3.18-3.04 (m, 2H), 3.00-2.94 (m, |
| | | | 1H), 2.39-2.33 (m, 1H), 1.84-1.78 (m, 2H), 1.57-1.53 (m, |
| | | | 1H) |
| 257 | 4-borono-1-naphthoic | 1 | (DMSO-d₆) δ 13.57 (br s, 1H), 13.26 (br s, 1H), 8.94 (d, |
| | acid (commercial) | | J = 8.6 Hz, 1H), 8.76 (br d, J = 8.1 Hz, 1H), 8.48 (s, 1H), 8.25 |
| | | | (d, J = 7.7 Hz, 1H), 8.17 (d, J = 7.5 Hz, 1H), 7.71-7.59 (m, |
| | | | 5H), 7.49 (br t, J = 7.3 Hz, 1H), 7.41-7.35 (m, 1H), 7.25-7.19 |
| | | | (m, 2H), 4.49 (br d, J = 14.3 Hz, 1H), 4.36 (br d, J = 10.1 Hz, |
| | | | 1H), 3.78-3.72 (m, 1H), 3.20-3.03 (m, 2H), 2.99-2.93 (m, |
| | | | 1H), 2.38-2.32 (m, 1H), 1.84-1.77 (m, 2H), 1.57-1.53 (m, |
| | | | 1H) |
| 258 | tert-butyl (6-(4,4,5,5- | 1 | (DMSO-d₆) δ 13.81 (br s, 1H), 13.57 (s, 1H), 9.11 (s, 1H), |
| | tetramethyl-1,3,2- | | 9.09 (br s, 1H), 8.99 (d, J = 8.8 Hz, 1H), 8.52 (s, 1H), 8.43 |
| | dioxaborolan-2- | | (d, J = 6.8 Hz, 1H), 8.03 (d, J = 8.8 Hz, 1H), 7.73 (br s, 3H), |
| | yl)quinolin-4- | | 7.50 (d, J = 7.0 Hz, 1H), 7.41-7.35 (m, 1H), 7.24-7.19 (m, |
| | yl)carbamate | | 2H), 6.81 (d, J = 6.8 Hz, 1H), 4.41 (br d, J = 13.8 Hz, 1H), |
| | | | 4.33 (br d, J = 12.5 Hz, 1H), 3.79 (br dd, J = 14.4, 6.5 Hz, |
| | | | 1H), 3.18-3.05 (m, 2H), 3.02-2.95 (m, 1H), 2.38-2.33 (m, |
| | | | 1H), 1.85-1.79 (m, 2H), 1.57-1.53 (m, 1H) |
| 259 | 4-methyl-5-(4,4,5,5- | 1 | (DMSO-d₆) δ 13.08 (br s, 1H), 10.77 (s, 1H), 10.69 (s, 1H), |
| | tetramethyl-1,3,2- | | 8.38 (s, 1H), 7.65 (br s, 3H), 7.49-7.46 (m, 2H), 7.41-7.34 |
| | dioxaborolan-2-yl)- | | (m, 1H), 7.24-7.18 (m, 2H), 6.88 (d, J = 8.1 Hz, 1H), 4.44 |
| | 1,3-dihydro-2H- | | (br d, J = 14.7 Hz, 1H), 4.32 (br d, J = 9.9 Hz, 1H), 3.69 (br |
| | benzo[d]imidazol-2- | | dd, J = 14.5, 6.6 Hz, 1H), 3.16-3.03 (m, 2H), 2.93-2.89 (m, |
| | one | | 1H), 2.41 (s, 3H), 2.38-2.32 (m, 1H), 1.81-1.76 (m, 2H), |
| | | | 1.55-1.51 (m, 1H) |
| 260 | 7-(4,4,5,5-tetramethyl- | 1 | (DMSO-d₆) δ 13.57 (s, 1H), 10.12 (d, J = 1.8 Hz, 1H), 8.66 |
| | 1,3,2-dioxaborolan-2- | | (dd, J = 9.2, 2.0 Hz, 1H), 8.61 (s, 1H), 8.34 (d, J = 6.4 Hz, |
| | yl)-4H-pyrido[1,2- | | 1H), 7.85 (d, J = 9.4 Hz, 1H), 7.66 (br s, 3H), 7.49 (t, J = 7.0 |
| | a]pyrimidin-4-one | | Hz, 1H), 7.41-7.36 (m, 1H), 7.25-7.19 (m, 2H), 6.47 (d, |
| | | | J = 6.4 Hz, 1H), 4.48 (br d, J = 14.3 Hz, 1H), 4.37 (br d, |
| | | | J = 12.7 Hz, 1H), 3.78 (br dd, J = 14.4, 6.5 Hz, 1H), 3.19-2.94 |
| | | | (m, 3H), 2.39-2.33 (m, 1H), 1.84-1.77 (m, 2H), 1.57-1.53 |
| | | | (m, 1H) |
| 261 | 1,3,4-trimethyl-5- | 1 | (DMSO-d₆) δ 13.18 (br s, 1H), 8.39 (s, 1H), 7.66 (br s, 3H), |
| | (4,4,5,5-tetramethyl- | | 7.48 (t, J = 7.0 Hz, 1H), 7.41-7.35 (m, 2H), 7.24-7.19 (m, |
| | 1,3,2-dioxaborolan-2- | | 2H), 7.10 (d, J = 8.1 Hz, 1H), 4.45 (br d, J = 13.6 Hz, 1H), |
| | yl)-1,3-dihydro-2H- | | 4.33 (br d, J = 9.7 Hz, 1H), 3.70 (br dd, J = 14.3, 6.4 Hz, 1H), |
| | benzo[d]imidazol-2- | | 3.65 (s, 3H), 3.36 (s, 3H), 3.19-3.05 (m, 2H), 2.94-2.89 (m, |
| | one | | 1H), 2.63 (s, 3H), 2.36-2.30 (m, 1H), 1.83-1.77 (m, 2H), |
| | | | 1.56-1.52 (m, 1H) |
| 262 | 3-methoxy-7-(4,4,5,5- | 1 | (DMSO-d₆) δ 13.50 (s, 1H), 9.42 (s, 1H), 8.61 (s, 1H), 8.57 |
| | tetramethyl-1,3,2- | | (d, J = 8.9 Hz, 1H), 8.05 (d, J = 9.0 Hz, 1H), 7.72 (s, 1H), 7.66 |
| | dioxaborolan-2- | | (br s, 2H), 7.49 (br t, J = 7.0 Hz, 1H), 7.41-7.36 (m, 1H), |
| | yl)cinnoline | | 7.25-7.19 (m, 2H), 4.48 (br d, J = 14.5 Hz, 1H), 4.38 (br d, |
| | | | J = 11.6 Hz, 1H), 4.20 (s, 3H), 3.77 (br dd, J = 14.1, 6.5 Hz, |
| | | | 1H), 3.19-3.03 (m, 2H), 3.00-2.95 (m, 1H), 2.40-2.33 (m, |
| | | | 1H), 1.85-1.79 (m, 2H), 1.58-1.54 (m, 1H) |
| 272 | 5-(4,4,5,5-tetramethyl- | 1 | (DMSO-d₆) δ 13.52 (br s, 1H), 8.57 (d, J = 9.9 Hz, 1H), 8.47 |
| | 1,3,2-dioxaborolan-2- | | (s, 1H), 7.86 (d, J = 7.5 Hz, 1H), 7.77 (t, J = 8.0 Hz, 1H), 7.71- |
| | yl)quinolin-2(1H)-one | | 7.62 (m, 4H), 7.49 (br t, J = 7.2 Hz, 1H), 7.41-7.36 (m, 1H), |
| | | | 7.25-7.19 (m, 2H), 6.66 (d, J = 9.9 Hz, 1H), 4.47 (br d, |
| | | | J = 14.3 Hz, 1H), 4.35 (br d, J = 9.7 Hz, 1H), 3.77-3.72 (m, |
| | | | 1H), 3.71 (s, 3H), 3.19-3.03 (m, 2H), 2.99-2.93 (m, 1H), |
| | | | 2.38-2.31 (m, 1H), 1.83-1.77 (m, 2H), 1.57-1.53 (m, 1H) |
| 274 | 5-(4,4,5,5-tetramethyl- | 1 | (DMSO-d₆) δ 13.46 (s, 1H), 8.47 (s, 1H), 8.42 (d, J = 6.8 Hz, |
| | 1,3,2-dioxaborolan-2- | | 1H), 8.35 (d, J = 8.3 Hz, 1H), 7.68-7.63 (m, 4H), 7.51-7.43 |
| | yl)isoquinolin-1(2H)- | | (m, 3H), 7.41-7.35 (m, 1H), 7.25-7.20 (m, 2H), 4.47 (br d, |
| | one | | J = 15.1 Hz, 1H), 4.35 (br d, J = 9.9 Hz, 1H), 3.75 (br dd, |
| | | | J = 14.1, 6.3 Hz, 1H), 3.54 (s, 3H), 3.19-3.03 (m, 2H), 2.99- |
| | | | 2.93 (m, 1H), 2.39-2.32 (m, 1H), 1.85-1.77 (m, 2H), 1.57- |
| | | | 1.53 (m, 1H) |
| 278 | Intermediate 164 | 1 | (DMSO-d₆) δ 13.73 (s, 1H), 8.50 (s, 1H), 7.87 (d, J = 7.7 Hz, |
| | | | 1H), 7.64 (br s, 3H), 7.48 (br t, J = 6.9 Hz, 1H), 7.41-7.35 |
| | | | (m, 1H), 7.24-7.19 (m, 2H), 6.73 (d, J = 7.5 Hz, 1H), 4.43 |
| | | | (br d, J = 14.7 Hz, 1H), 4.33 (br d, J = 10.1 Hz, 1H), 3.77- |
| | | | 3.72 (m, 1H), 3.66 (s, 3H), 3.17-3.02 (m, 2H), 2.99-2.92 |
| | | | (m, 1H), 2.37-2.30 (m, 1H), 1.83-1.75 (m, 2H), 1.57-1.52 |
| | | | (m, 1H) |
| 280 | Intermediate 179a | 2 | (DMSO-d₆) δ 13.35 (br s, 1H), 8.61 (br s, 1H), 8.42 (br s, |
| | | | 1H), 7.80-7.59 (m, 5H), 7.48 (br d, J = 5.7 Hz, 1H), 7.40- |
| | | | 7.31 (m, 1H), 7.27-7.11 (m, 2H), 4.92 (br dd, J = 3.1, 6.6 Hz, |
| | | | 1H), 4.54-4.41 (m, 1H), 4.39-4.29 (m, 1H), 3.77-3.66 (m, |
| | | | 1H), 3.20-3.02 (m, 2H), 3.00-2.86 (m, 1H), 2.41-2.26 (m, |
| | | | 1H), 1.87-1.74 (m, 2H), 1.63-1-57 (m, 7H) |
| 288 | 3-(4,4,5,5-tetramethyl- | 2 | (DMSO-d₆) δ 13.57 (s, 1H), 9.68 (s, 1H), 9.00 (s, 1H), 8.83 |
| | 1,3,2-dioxaborolan-2- | | (s, 1H), 8.55 (s, 1H), 7.66 (br s, 3H), 7.51-7.36 (m, 2H), |
| | yl)-5,6-dihydro-7H- | | 7.25-7.19 (m, 2H), 4.49 (br s, 2H), 4.47-4.44 (br m, 1H), |
| | pyrrolo[3,4-b]pyridin- | | 4.38-4.34 (brm, 1H), 3.82-3.75 (m, 1H), 3.16-2.97 (m, 3H), |
| | 7-one | | 2.38-2.31 (m, 1H), 1.85-1.78 (m, 2H), 1.58-1.54 (m, 1H) |
| 289 | 1-methyl-5-(4,4,5,5- | 2 | (DMSO-d₆) δ 13.09 (br s, 1H), 10.98 (s, 1H), 8.45 (s, 1H), |
| | tetramethyl-1,3,2- | | 8.08 (dd, J = 8.2, 1.4 Hz, 1H), 8.03 (d, J = 1.1 Hz, 1H), 7.67 |
| | dioxaborolan-2-yl)- | | (br s, 3H), 7.49 (br t, J = 7.0 Hz, 1H), 7.41-7.36 (m, 1H), |
| | 1,3-dihydro-2H- | | 7.25-7.19 (m, 3H), 4.45 (br d, J = 13.6 Hz, 1H), 4.34 (br d, |
| | benzo[d]imidazol-2- | | J = 9.0 Hz, 1H), 3.76-3.71 (m, 1H), 3.32 (s, 3H), 3.19-3.04 |
| | one | | (m, 2H), 2.98-2.91 (m, 1H), 2.39-2.32 (m, 1H), 1.84-1.77 |
| | | | (m, 2H), 1.56-1.52 (m, 1H) |
| 290 | 6-(4,4,5,5-tetramethyl- | 2 | (DMSO-d₆) δ 13.52 (s, 1H), 9.74 (s, 1H), 9.33 (s, 1H), 9.12 |
| | 1,3,2-dioxaborolan-2- | | (s, 1H), 8.97-8.94 (m, 1H), 8.57 (s, 1H), 8.18-8.15 (m, 1H), |
| | yl)quinazoline | | 7.65 (br s, 3H), 7.51-7.48 (m, 1H), 7.41-7.35 (m, 1H), 7.25- |
| | (commercial) | | 7.20 (m, 2H), 4.49-4.45 (m, 1H), 4.39-4.35 (m, 1H), 3.79 |
| | | | (dd, J = 14.8, 6.3 Hz, 1H), 3.19-2.93 (m, 3H), 2.39-2.35 (m, |
| | | | 1H), 1.85-1.80 (m, 2H), 1.56-1.52 (m, 1H) |
| 292 | 4-methoxy-6-(4,4,5,5- | 2 | Method 2: m/z 501 (M + H)⁺, RT 1.02 min |
| | tetramethyl-1,3,2- | | |
| | dioxaborolan-2-yl)- | | |
| | 1,3-dihydro-2H- | | |
| | benzo[d]imidazol-2- | | |
| | one | | |
| 293 | 5-(4,4,5,5-tetramethyl- | 2 | Method 2: m/z 484 (M + H)⁺, RT 1.02 min. |
| | 1,3,2-dioxaborolan-2- | | |
| | yl)-3,4- | | |
| | dihydroquinolin- | | |
| | 2(1H)-one | | |
| 294 | 7-(4,4,5,5-tetramethyl- | 2 | Method 2: m/z 482 (M + H)⁺, RT 1.10 min |
| | 1,3,2-dioxaborolan-2- | | |
| | yl)isoquinolin-1(2H)- | | |
| | one | | |
| 298 | 5-(4,4,5,5-tetramethyl- | 2 | Method 2: m/z 484 (M + H)⁺, RT 1.01 min. |
| | 1,3,2-dioxaborolan-2- | | |
| | yl)-3,4- | | |
| | dihydroisoquinolin- | | |
| | 1(2H)-one | | |
| 299 | 5-fluoro-6-(4,4,5,5- | 2 | (DMSO-d₆) 13.60 (br s, 1H), 11.52 (br d, J = 4.6 Hz, 1H), |
| | tetramethyl-1,3,2- | | 8.51 (s, 1H), 8.27-8.23 (m, 1H), 8.13 (d, J = 8.3 Hz, 1H), |
| | dioxaborolan-2- | | 7.68 (br s, 3H), 7.49 (br t, J = 7.7 Hz, 1H), 7.41-7.36 (m, |
| | yl)isoquinolin-1(2H)- | | 1H), 7.33 (t, J = 6.3 Hz, 1H), 7.25-7.19 (m, 2H), 6.70 (d, |
| | one | | J = 7.0 Hz, 1H), 4.47 (br d, J = 14.0 Hz, 1H), 4.35 (br d, |
| | | | J = 12.7 Hz, 1H), 3.76 (dd, J = 14.3, 6.4 Hz, 1H), 3.18-3.02 |
| | | | (m, 2H), 3.00-2.92 (m, 1H), 2.39-2.32 (m, 1H), 1.84-1.76 |
| | | | (m, 2H), 1.57-1.53 (m, 1H) |
| 300 | 2-methyl-6-(4,4,5,5- | 2 | (DMSO-d₆) 13.48 (br s, 1H), 8.61 (s, 1H), 8.54 (s, 1H), |
| | tetramethyl-1,3,2- | | 8.45 (dd, J = 8.4, 1.4 Hz, 1H), 8.33 (d, J = 8.6 Hz, 1H), 7.69 |
| | dioxaborolan-2- | | (br s, 3H), 7.53-7.47 (m, 2H), 7.41-7.36 (m, 1H), 7.25-7.19 |
| | yl)isoquinolin-1(2H)- | | (m, 2H), 6.71 (d, J = 7.2 Hz, 1H), 4.46 (br d, J = 14.0 Hz, 1H), |
| | one | | 4.35 (br d, J = 10.7 Hz, 1H), 3.77 (dd, J = 14.3, 6.4 Hz, 1H), |
| | | | 3.54 (s, 3H), 3.18-3.04 (m, 2H), 3.00-2.94 (m, 1H), 2.39- |
| | | | 2.33 (m, 1H), 1.85-1.77 (m, 2H), 1.57-1.53 (m, 1H) |
| 301 | 7-(4,4,5,5-tetramethyl- | 2 | Method 2: m/z 483 (M + H)⁺, RT 1.04 min |
| | 1,3,2-dioxaborolan-2- | | |
| | yl)quinazolin-4(3H)- | | |
| | one | | |
| 302 | Intermediate 166 | 2 | Method 2: m/z 505 (M + H)⁺, RT 0.98 min. |
| 303 | 5-(4,4,5,5-tetramethyl- | 2 | Method 2: m/z 471 (M + H)⁺, RT 0.98 min. |
| | 1,3,2-dioxaborolan-2- | | |
| | yl)-1,3-dihydro-2H- | | |
| | pyrrolo[2,3-b]pyridin- | | |
| | 2-one | | |
| 304 | 1-(5-(4,4,5,5- | 2 | (DMSO-d₆) δ 13.10 (br s, 1H), 8.45 (s, 1H), 8.19 (s, 1H), |
| | tetramethyl-1,3,2- | | 8.18-8.12 (m, 2H), 7.67 (br s, 3H), 7.49 (br t, J = 7.0 Hz, |
| | dioxaborolan-2- | | 1H), 7.40-7.35 (m, 1H), 7.24-7.19 (m, 2H), 4.44 (br d, |
| | yl)indolin-1-yl)ethan- | | J = 14.0 Hz, 1H), 4.33 (br d, J = 10.3 Hz, 1H), 4.15 (br t, |
| | 1-one | | J = 8.3 Hz, 2H), 3.83-3.50 (m, 1H), 3.23 (brt, J = 8.6 Hz, 2H), |
| | | | 3.17-3.03 (m, 2H), 2.97-2.91 (m, 1H), 2.39-2.31 (m, 1H), |
| | | | 2.19 (s, 3H), 1.84-1.78 (m, 2H), 1.56-1.52 (m, 1H) |
| 305 | 1-methyl-6-(4,4,5,5- | 1 | (DMSO-d₆) δ 13.30 (br s, 1H), 8.76 (br s, 1H), 8.61 (s, 1H), |
| | tetramethyl-1,3,2- | | 8.53 (s, 1H), 8.43 (br d, J = 8.1 Hz, 1H), 7.86 (br d, J = 8.6 |
| | dioxaborolan-2-yl)- | | Hz, 1H), 7.66 (br s, 3H), 7.49 (br t, J = 7.7 Hz, 1H), 7.41- |
| | 1H-benzo[d]imidazole | | 7.36 (m, 1H), 7.25-7.20 (m, 2H), 4.45 (br d, J = 14.0 Hz, |
| | | | 1H), 4.35 (br d, J = 9.7 Hz, 1H), 4.00 (s, 3H), 3.79-3.74 (m, |
| | | | 1H), 3.21-3.04 (m, 2H), 3.01-2.94 (m, 1H), 2.40-2.35 (m, |
| | | | 1H), 1.86-1.78 (m, 2H), 1.57-1.53 (m, 1H) |
| 309 | 6-(4,4,5,5-tetramethyl- | 1 | Method 2: m/z 535 (M + H)⁺, RT 1.45 min |
| | 1,3,2-dioxaborolan-2- | | |
| | yl)-2- | | |
| | (trifluoromethyl) | | |
| | quinazoline | | |
| 311 | 5-chloro-6-(4,4,5,5- | 2 | (DMSO-d₆) δ 13.02 (br s, 1H), 8.21 (s, 1H), 7.49 (br s, 3H), |
| | tetramethyl-1,3,2- | | 7.32 (br t, J = 7.7 Hz, 1H), 7.25-7.19 (m, 1H), 7.08-7.03 (m, |
| | dioxaborolan-2-yl)- | | 2H), 6.67 (d, J = 8.3 Hz, 1H), 6.61 (d, J = 8.3 Hz, 1H), 4.28 |
| | 3,4-dihydro-2H- | | (br d, J = 14.7 Hz, 1H), 4.16 (br d, J = 10.1 Hz, 1H), 4.02 (br |
| | benzo[b][1,4]oxazine | | t, J = 4.3 Hz, 2H), 3.55-3.50 (m, 1H), 3.46-3.38 (m, 2H), |
| | | | 3.02-2.90 (m, 2H), 2.79-2.71 (m, 1H), 2.21-2.13 (m, 1H), |
| | | | 1.69-1.60 (m, 2H), 1.40-1.36 (m, 1H) |
| 312 | tert-butyl 5-(4,4,5,5- | 2 | Method 2: m/z 456 (M + H)⁺, RT 0.93 min |
| | tetramethyl-1,3,2- | | |
| | dioxaborolan-2- | | |
| | yl)indoline-1- | | |
| | carboxylate | | |
| 314 | 8-fluoro-5-(4,4,5,5- | 2 | Method 2: m/z 500 (M + H)⁺, RT 1.03 min |
| | tetramethyl-1,3,2- | | |
| | dioxaborolan-2- | | |
| | yl)isoquinolin-1(2H)- | | |
| | one | | |
| 317 | Intermediate 165 | 2 | Method 2: m/z 533 (M + H)⁺, RT 1.06 min |
| 318 | 1,4-dimethyl-5- | 2 | Method 2: m/z 483 (M + H)⁺, RT 0.91 min |
| | (4,4,5,5-tetramethyl- | | |
| | 1,3,2-dioxaborolan-2- | | |
| | yl)-1H- | | |
| | benzo[d]imidazole | | |
| | (commercial) | | |
| 319 | 6-(4,4,5,5-tetramethyl- | 2 | Method 2: m/z 481 (M + H)⁺, RT 0.92 min |
| | 1,3,2-dioxaborolan-2- | | |
| | yl)isoquinolin-1-amine | | |
| 320 | 1-methyl-6-(4,4,5,5- | 2 | Method 2: m/z 485 (M + H)⁺, RT 1.06 min |
| | tetramethyl-1,3,2- | | |
| | dioxaborolan-2-yl)- | | |
| | 1,3-dihydro-2H- | | |
| | benzo[d]imidazol-2- | | |
| | one | | |
| 321 | Intermediate 167 | 2 | (DMSO-d₆) δ 13.19 (br s, 1H), 8.39 (s, 1H), 7.67 (br s, 3H), |
| | | | 7.49 (br t, J = 7.1 Hz, 1H), 7.41-7.36 (m, 1H), 7.25-7.19 (m, |
| | | | 3H), 6.94 (d, J = 8.6 Hz, 1H), 4.44 (br d, J = 14.0 Hz, 1H), |
| | | | 4.33 (br d, J = 10.1 Hz, 1H), 4.24-4.22 (m, 2H), 3.70 (dd, |
| | | | J = 14.3, 6.4 Hz, 1H), 3.19-3.06 (m, 4H), 2.95-2.88 (m, 1H), |
| | | | 2.83 (s, 3H), 2.37-2.30 (m, 1H), 1.85-1.77 (m, 2H), 1.56- |
| | | | 1.52 (m, 1H) |
| 322 | 2,6-difluoro-4- | 2 | (DMSO-d₆) δ 13.27 (br s, 1H), 10.47 (br s, 1H), 8.48 (s, |
| | (4,4,5,5-tetramethyl- | | 1H), 7.94 (br d, J = 8.1 Hz, 2H), 7.65 (br s, 3H), 7.48 (br t, |
| | 1,3,2-dioxaborolan-2- | | J = 7.1 Hz, 1H), 7.41-7.36 (m, 1H), 7.25-7.19 (m, 2H), 4.44 |
| | yl)phenol | | (br d, J = 14.3 Hz, 1H), 4.33 (br d, J = 10.1 Hz, 1H), 3.74 (dd, |
| | | | J = 14.0, 6.4 Hz, 1H), 3.17-3.03 (m, 2H), 2.98-2.92 (m, 1H), |
| | | | 2.40-2.38 (m, 1H), 1.83-1.76 (m, 2H), 1.56-1.52 (m, 1H) |
| 325 | Intermediate 179b | 2 | (DMSO-d₆) δ 13.37 (br s, 1H), 8.44 (s, 1H), 8.21 (s, 1H), |
| | | | 7.87-7.81 (m, 1H), 7.80-7.74 (m, 1H), 7.71-7.60 (m, 3H), |
| | | | 7.53-7.45 (m, 1H), 7.42-7.34 (m, 1H), 7.27-7.16 (m, 2H), |
| | | | 5.18-5.00 (m, 1H), 4.47 (br d, J = 14.3 Hz, 1H), 4.40-4.32 |
| | | | (m, 1H), 3.72 (br dd, J = 6.4, 14.5 Hz, 1H), 3.20-3.04 (m, |
| | | | 2H), 2.94 (br d, J = 4.2 Hz, 1H), 2.41-2.29 (m, 1H), 1.86- |
| | | | 1.74 (m, 2H), 1.54 (d, J = 6.6 Hz, 7H) |
| 326 | Intermediate 169 | 2 | Method 2: m/z 532 (M + H)⁺, RT 1.17 min |
| 327 | 6-(4,4,5,5-tetramethyl- | 2 | Method 2: m/z 456 (M + H)⁺, RT 1.09 min |
| | 1,3,2-dioxaborolan-2- | | |
| | yl)-[1,2,4]triazolo[1,5- | | |
| | a]pyridine | | |
| 329 | 4-chloro-1-methyl-5- | 2 | Method 2: m/z 503 (M + H)⁺, RT 0.98 min |
| | (4,4,5,5-tetramethyl- | | |
| | 1,3,2-dioxaborolan-2- | | |
| | yl)-1H- | | |
| | benzo[d]imidazole | | |
| 330 | (3,5- | 2 | Method 2: m/z 451 (M + H)⁺, RT 1.36 min |
| | difluorophenyl)boronic | | |
| | acid (commercial) | | |
| 331 | 2-fluoro-4-(4,4,5,5- | 2 | (DMSO-d₆) δ 13.06 (br s, 1H), 10.01 (br s, 1H), 8.38 (s, |
| | tetramethyl-1,3,2- | | 1H), 7.98 (br d, J = 12.7 Hz, 1H), 7.92 (br d, J = 8.6 Hz, 1H), |
| | dioxaborolan-2- | | 7.59 (br s, 3H), 7.42 (br t, J = 7.3 Hz, 1H), 7.33-7.29 (m, |
| | yl)phenol | | 1H), 7.17-7.12 (m, 2H), 7.00 (t, J = 8.8 Hz, 1H), 4.38 (br d, |
| | | | J = 14.0 Hz, 1H), 4.26 (br d, J = 12.7 Hz, 1H), 3.65 (dd, |
| | | | J = 14.0, 6.4 Hz, 1H), 3.11-2.96 (m, 2H), 2.90-2.82 (m, 1H), |
| | | | 2.33-2.29 (m, 1H), 1.76-1.71 (m, 2H), 1.49-1.45 (m, 1H) |
| 332 | 3-fluoro-4-(4,4,5,5- | 2 | Method 2: m/z 449 (M + H)⁺, RT 1.05 min |
| | tetramethyl-1,3,2- | | |
| | dioxaborolan-2- | | |
| | yl)phenol | | |
| 333 | 7-(4,4,5,5-tetramethyl- | 2 | Method 2: m/z 456 (M + H)⁺, RT 1.05 min |
| | 1,3,2-dioxaborolan-2- | | |
| | yl)-[1,2,4]triazolo[1,5- | | |
| | a]pyridine | | |
| 335 | Intermediate 137c | 2 | (DMSO-d₆) δ 13.47 (br s, 1H), 9.12 (s, 1H), 8.46 (s, 1H), |
| | | | 8.32-8.03 (m, 1H), 7.85 (s, 2H), 7.67 (br s, 3H), 7.49 (br t, |
| | | | J = 7.6 Hz, 1H), 7.41-7.36 (m, 1H), 7.25-7.20 (m, 2H), 4.46 |
| | | | (br d, J = 14.7 Hz, 1H), 4.35 (br d, J = 9.7 Hz, 1H), 3.76-3.71 |
| | | | (m, 1H), 3.20-3.03 (m, 2H), 2.99-2.90 (m, 1H), 2.39-2.31 |
| | | | (m, 1H), 1.84-1.77 (m, 2H), 1.57-1.53 (m, 1H) |
| 337 | 2-methyl-5-(4,4,5,5- | 2 | Method 2: m/z 469 (M + H)⁺, RT 0.90 min |
| | tetramethyl-1,3,2- | | |
| | dioxaborolan-2-yl)- | | |
| | 1H-benzo[d]imidazole | | |
| 338 | 7-chloro-1-methyl-6- | 2 | (DMSO-d₆) δ 13.33 (br s, 1H), 8.42 (s, 1H), 8.33 (s, 1H), |
| | (4,4,5,5-tetramethyl- | | 7.72 (d, J = 8.3 Hz, 1H), 7.66 (br s, 3H), 7.50 (d, J = 8.3 Hz, |
| | 1,3,2-dioxaborolan-2- | | 1H), 7.49-7.47 (m, 1H), 7.41-7.36 (m, 1H), 7.25-7.20 (m, |
| | yl)-1H- | | 2H), 4.47-4.44 (m, 1H), 4.36-4.32 (m, 1H), 4.16 (s, 3H), |
| | benzo[d]imidazole | | 3.74-3.69 (m, 1H), 3.20-3.05 (m, 2H), 2.99-2.90 (m, 1H), |
| | | | 2.40-2.32 (m, 1H), 1.84-1.77 (m, 2H), 1.58-1.54 (m, 1H) |
| 339 | Intermediate 170 | 2 | (DMSO-d₆) δ 13.66 (s, 1H), 8.68 (s, 1H), 8.48 (s, 1H), 8.36 |
| | | | (d, J = 8.3 Hz, 1H), 8.25 (d, J = 8.3 Hz, 1H), 7.66 (br s, 3H), |
| | | | 7.49 (d, J = 7.1 Hz, 1H), 7.42-7.36 (m, 1H), 7.25-7.19 (m, |
| | | | 2H), 4.45 (d, J = 14.7 Hz, 1H), 4.36-4.33 (m, 1H), 3.79 (s, |
| | | | 3H), 3.77-3.73 (m, 1H), 3.20-3.05 (m, 2H), 3.05-3.03 (m, |
| | | | 1H), 2.40-2.32 (m, 1H), 1.84-1.77 (m, 2H), 1.57-1.54 (m, |
| | | | 1H) |
| 340 | 5-chloro-2-methyl-6- | 2 | (DMSO-d₆) δ 13.66 (br s, 1H), 13.03 (br s, 1H), 8.65 (br s, |
| | (4,4,5,5-tetramethyl- | | 1H), 8.48 (br s, 1H), 8.35-8.30 (br m, 1H), 8.25-8.21 (br m, |
| | 1,3,2-dioxaborolan-2- | | 1H), 7.65 (br s, 3H), 7.52-7.46 (br m, 1H), 7.42-7.36 (m, |
| | yl)phthalazin-1(2H)- | | 1H), 7.25-7.19 (m, 2H), 4.46 (d, J = 13.6 Hz, 1H), 4.37-4.32 |
| | one | | (m, 1H), 3.79-3.72 (m, 1H), 3.19-3.04 (m, 2H), 3.01-2.93 |
| | | | (m, 1H), 2.39-2.31 (m, 1H), 1.85-1.77 (m, 2H), 1.59-1.54 |
| | | | (m, 1H) |
| 341 | 3-methyl-5-(4,4,5,5- | 2 | Method 2: m/z 486 (M + H)⁺, RT 1.23 min |
| | tetramethyl-1,3,2- | | |
| | dioxaborolan-2- | | |
| | yl)benzo[d]oxazol- | | |
| | 2(3H)-one | | |
| 345 | 5-(4,4,5,5-tetramethyl- | 2 | Method 2: m/z 500 (M + H)⁺, RT 1.05 min |
| | 1,3,2-dioxaborolan-2- | | |
| | yl)-6- | | |
| | (trifluoromethyl)pyridin- | | |
| | 2-ol | | |
| 346 | 3-chloro-4-(4,4,5,5- | 2 | Method 2: m/z 528 (M + H)⁺, RT 1.07 min |
| | tetramethyl-1,3,2- | | |
| | dioxaborolan-2- | | |
| | yl)benzenesulfonamide | | |
| 347 | 1-methyl-4-(4,4,5,5- | 2 | Method 2: m/z 419 (M + H)⁺, RT 1.00 min |
| | tetramethyl-1,3,2- | | |
| | dioxaborolan-2-yl)- | | |
| | 1H-pyrazole | | |
| | (commercial) | | |
| 348 | (1,5-dimethyl-1H- | 2 | Method 2: m/z 433 (M + H)⁺, RT 1.03 min |
| | pyrazol-4-yl)boronic | | |
| | acid (commercial) | | |
| 349 | Intermediate 171 | 2 | Method 2: m/z 517 (M + H)⁺, RT 0.89 min |
| 350 | Intermediate 172 | 2 | (DMSO-d₆) δ 13.52 (s, 1H), 8.46 (s, 1H), 7.99 (d, J = 7.9 Hz, |
| | | | 1H), 7.78 (d, J = 7.9 Hz, 1H), 7.66 (br s, 3H), 7.49 (br t, |
| | | | J = 7.5 Hz, 1H), 7.42-7.36 (m, 1H), 7.25-7.19 (m, 2H), 4.57 |
| | | | (s, 2H), 4.45 (br d, J = 14.3 Hz, 1H), 4.34 (br d, J = 9.4 Hz, |
| | | | 1H), 3.44 (br dd, J = 13.9, 6.3 Hz, 1H), 3.14 (s, 3H), 3.13- |
| | | | 3.04 (m, 2H), 2.99-2.92 (m, 1H), 2.39-2.31 (m, 1H), 1.84- |
| | | | 1.77 (m, 2H), 1.58-1.54 (m, 1H) |
| 351 | Intermediate 173 | 2 | (DMSO-d₆) δ 13.59 (s, 1H), 8.51 (s, 1H), 8.26 (t, J = 6.7 Hz, |
| | | | 1H), 8.17 (d, J = 8.6 Hz, 1H), 7.64 (br s, 3H), 7.57 (d, J = 8.3 |
| | | | Hz, 1H), 7.49 (brt, J = 6.8 Hz, 1H), 7.41-7.36 (m, 1H), 7.25- |
| | | | 7.19 (m, 2H), 6.75 (d, J = 7.5 Hz, 1H), 4.91 (t, J = 5.0 Hz, |
| | | | 1H), 4.49-4.45 (m, 1H), 4.38-4.33 (m, 1H), 4.07 (t, J = 5.3 |
| | | | Hz, 2H), 3.79-3.70 (m, 3H), 3.16-2.92 (m, 3H), 2.40-2.32 |
| | | | (m, 1H), 1.86-1.77 (m, 2H), 1.58-1.54 (m, 1H) |
| 352 | 1-methoxy-6-(4,4,5,5- | 2 | Method 2: m/z 496 (M + H)⁺, RT 1.40 min |
| | tetramethyl-1,3,2- | | |
| | dioxaborolan-2- | | |
| | yl)isoquinoline | | |
| 353 | 3-methyl-6-(4,4,5,5- | 2 | Method 2: m/z 486 (M + H)⁺, RT 1.19 min |
| | tetramethyl-1,3,2- | | |
| | dioxaborolan-2- | | |
| | yl)benzo[d]oxazol- | | |
| | 2(3H)-one | | |
| 354 | 2,5-difluoro-4- | 2 | (DMSO-d₆) δ 13.27 (s, 1H), 10.67 (br s, 1H), 8.45 (s, 1H), |
| | (4,4,5,5-tetramethyl- | | 7.97 (dd, J = 12.0, 6.7 Hz, 1H), 7.67 (br s, 3H), 7.49 (br t, |
| | 1,3,2-dioxaborolan-2- | | J = 7.5 Hz, 1H), 7.41-7.36 (m, 1H), 7.24-7.19 (m, 2H), 6.91 |
| | yl)phenol | | (dd, J = 11.3, 7.6 Hz, 1H), 4.45 (br d, J = 14.3 Hz, 1H), 4.33 |
| | | | (br d, J = 9.2 Hz, 1H), 3.75-3.70 (m, 1H), 3.17-3.04 (m, 2H), |
| | | | 2.97-2.91 (m, 1H), 2.39-2.31 (m, 1H), 1.84-1.77 (m, 2H), |
| | | | 1.56-1.52 (m, 1H) |
| 355 | 2,6-difluoro-3- | 2 | Method 2: m/z 467 (M + H)⁺, RT 1.15 min |
| | (4,4,5,5-tetramethyl- | | |
| | 1,3,2-dioxaborolan-2- | | |
| | yl)phenol | | |
| 357 | Intermediate 174 | 2 | (DMSO-d₆) δ 13.60 (br s, 1H), 8.51 (s, 1H), 8.28 (t, J = 7.5 |
| | 5-fluoro-2-(2- | | Hz, 1H), 8.17 (d, J = 8.6 Hz, 1H), 7.66 (br s, 3H), 7.57 (d, |
| | methoxyethyl)-6- | | J = 7.5 Hz, 1H), 7.49 (brt, J = 7.7 Hz, 1H), 7.41-7.36 (m, 1H), |
| | (4,4,5,5-tetramethyl- | | 7.25-7.19 (m, 2H), 6.77 (d, J = 7.5 Hz, 1H), 4.47 (br d, |
| | 1,3,2-dioxaborolan-2- | | J = 14.0 Hz, 1H), 4.36 (br d, J = 9.1 Hz, 1H), 4.19 (br t, J = 5.0 |
| | yl)isoquinolin-1(2H)- | | Hz, 2H), 3.78-3.74 (m, 1H), 3.66 (t, J = 5.2 Hz, 2H), 3.27 (s, |
| | one | | 3H), 3.19-3.03 (m, 2H), 3.00-2.93 (m, 1H), 2.39-2.31 (m, |
| | | | 1H), 1.84-1.77 (m, 2H), 1.57-1.53 (m, 1H) |
| 358 | Intermediate 175 | 2 | (DMSO-d₆) δ 13.33 (br s, 1H), 8.40 (s, 1H), 7.67 (br s. 3H), |
| | | | 7.65 (d, J = 8.3 Hz, 1H), 7.51-7.47 (m, 2H), 7.42-7.36 (m, |
| | | | 1H), 7.25-7.20 (m, 2H), 4.44 (br d, J = 14.3 Hz, 1H), 4.34 |
| | | | (br d, J = 12.3 Hz, 1H), 4.10 (s, 3H), 3.72 (br dd, J = 14.3, 7.0 |
| | | | Hz, 1H), 3.20-3.06 (m, 2H), 2.97-2.90 (m, 1H), 2.65 (s, |
| | | | 3H), 2.39-2.32 (m, 1H), 1.83-1.77 (m, 2H), 1.57-1.53 (m, |
| | | | 1H) |
| 359 | Intermediate 176 | 2 | (DMSO-d₆) δ 13.52 (br s, 1H), 8.53 (s, 1H), 8.13 (d, J = 8.8 |
| | | | Hz, 2H), 7.66 (br s, 3H), 7.49 (t, J = 7.5 Hz, 1H), 7.41-7.36 |
| | | | (m, 1H), 7.25-7.19 (m, 2H), 4.44 (br d, J = 13.8 Hz, 1H), |
| | | | 4.34 (br d, J = 12.3 Hz, 1H), 3.75 (br dd, J = 14.3, 6.4 Hz, |
| | | | 1H), 3.16-3.04 (m, 2H), 3.00-2.93 (m, 1H), 2.45 (s, 3H), |
| | | | 2.39-2.32 (m, 1H), 1.85-1.77 (m, 2H), 1.57-1.53 (m, 1H) |
| 360 | 4-(4,4,5,5-tetramethyl- | 2 | Method 2: m/z 499 (M + H)⁺, RT 1.31 min |
| | 1,3,2-dioxaborolan-2- | | |
| | yl)-2- | | |
| | (trifluoromethyl)phenol | | |
| 361 | 4-chloro-5-(4,4,5,5- | 2 | Method 2: m/z 571 (M + H)⁺, RT 1.42 min |
| | tetramethyl-1,3,2- | | |
| | dioxaborolan-2-yl)-2- | | |
| | (2,2,2-trifluoroethyl)- | | |
| | 2H-indazole (prepared | | |
| | as reported for | | |
| | Intermediate 45 using | | |
| | Cs₂CO₃ and 1,1,1- | | |
| | trifluoro-2-iodo-ethane | | |
| | in step 2) | | |
| 362 | 4-chloro-3,3-difluoro- | 2 | (DMSO-d₆) δ 13.46 (br s, 1H), 8.44 (s, 1H), 8.02 (d, J = 8.3 |
| | 1-methyl-5-(4,4,5,5- | | Hz, 1H), 7.67 (br s, 3H), 7.49 (t, J = 7.6 Hz, 1H), 7.41-7.35 |
| | tetramethyl-1,3,2- | | (m, 2H), 7.25-7.19 (m, 2H), 4.44 (br d, J = 14.0 Hz, 1H), |
| | dioxaborolan-2- | | 4.33 (br d, J = 11.0 Hz, 1H), 3.73 (br dd, J = 14.3, 6.4 Hz, |
| | yl)indolin-2-one | | 1H), 3.24 (s, 3H), 3.18-3.04 (m, 2H), 2.99-2.92 (m, 1H), |
| | | | 2.39-2.31 (m, 1H), 1.85-1.77 (m, 2H), 1.57-1.53 (m, 1H) |
| 363 | Intermediate 177a | 2 | (DMSO-d₆) δ 13.39 (br s, 1H), 8.54 (s, 1H), 8.44 (s, 1H), |
| | | | 7.78 (d, J = 8.6 Hz, 1H), 7.68 (br s, 3H), 7.49 (t, J = 7.7 Hz, |
| | | | 1H), 7.40-7.36 (m, 2H), 7.25-7.19 (m, 2H), 4.47 (br d, |
| | | | J = 14.3 Hz, 1H), 4.35 (br d, J = 11.0 Hz, 1H), 4.25 (s, 3H), |
| | | | 3.72 (br dd, J = 14.4, 6.5 Hz, 1H), 3.18-3.05 (m, 2H), 2.96- |
| | | | 2.91 (m, 1H), 2.38-2.31 (m, 1H), 1.87-1.77 (m, 2H), 1.57- |
| | | | 1.53 (m, 1H) |
| 364 | Intermediate 177b | 2 | (DMSO-d₆) δ 13.43 (br s, 1H), 8.43 (s, 1H), 8.21 (s, 1H), |
| | | | 7.84 (d, J = 8.3 Hz, 1H), 7.69 (br s, 3H), 7.50 (t, J = 7.7 Hz, |
| | | | 1H), 7.43-7.36 (m, 2H), 7.25-7.19 (m, 2H), 4.45 (br d, |
| | | | J = 14.3 Hz, 1H), 4.41 (s, 3H), 4.34 (br d, J = 10.7 Hz, 1H), |
| | | | 3.73 (br dd, J = 14.1, 6.5 Hz, 1H), 3.20-3.05 (m, 2H), 2.97- |
| | | | 2.91 (m, 1H), 2.38-2.32 (m, 1H), 1.85-1.77 (m, 2H), 1.57- |
| | | | 1.53 (m, 1H) |
| 365 | 2-(2-((tert- | 2 | (DMSO-d₆) δ 13.36 (s, 1H), 8.54 (s, 1H), 8.43 (s, 1H), 7.71- |
| | butyldimethylsilyl)oxy) | | 7.63 (m, 5H), 7.49 (t, J = 6.9 Hz, 1H), 7.42-7.36 (m, 1H), |
| | ethyl)-4-chloro-5- | | 7.25-7.19 (m, 2H), 5.04-5.01 (m, 1H), 4.54-4.52 (m, 2H), |
| | (4,4,5,5-tetramethyl- | | 4.47 (br d, J = 14.0 Hz, 1H), 4.37-4.33 (m, 1H), 3.95-3.90 |
| | 1,3,2-dioxaborolan-2- | | (m, 2H), 3.75-3.70 (m, 1H), 3.19-3.06 (m, 2H), 2.97-2.91 |
| | yl)-2H-indazole | | (m, 1H), 2.38-2.31 (m, 1H), 1.85-1.77 (m, 2H), 1.57-1.54 |
| | (prepared as reported | | (m, 1H) |
| | for Intermediate 106); | | |
| 367 | 3-fluoro-4-(4,4,5,5- | 2 | (DMSO-d₆) δ 13.52 (br s, 1H), 8.49 (s, 1H), 8.27 (t, J = 7.7 |
| | tetramethyl-1,3,2- | | Hz, 1H), 8.13 (br s, 1H), 7.88-7.81 (m, 2H), 7.68 (br s, 3H), |
| | dioxaborolan-2- | | 7.58 (br s, 1H), 7.49 (br t, J = 7.1 Hz, 1H), 7.41-7.36 (m, |
| | yl)benzamide | | 1H), 7.25-7.19 (m, 2H), 4.46 (br d, J = 14.3 Hz, 1H), 4.34 |
| | | | (br d, J = 9.7 Hz, 1H), 3.77-3.72 (m, 1H), 3.18-3.04 (m, 2H), |
| | | | 2.99-2.91 (m, 1H), 2.38-2.32 (m, 1H), 1.84-1.77 (m, 2H), |
| | | | 1.56-1.53 (m, 1H) |
| 368 | tert-butyl (tert- | 2 | Method 2: m/z 445 (M + H)⁺, RT 0.82 min |
| | butoxycarbonyl)(6- | | |
| | methyl-5-(4,4,5,5- | | |
| | tetramethyl-1,3,2- | | |
| | dioxaborolan-2- | | |
| | yl)pyridin-2- | | |
| | yl)carbamate | | |
| 370 | 7-chloro-6-(4,4,5,5- | 2 | (DMSO-d₆) δ 13.53 (br s, 1H), 11.49 (br s, 1H), 8.47 (s, |
| | tetramethyl-1,3,2- | | 1H), 8.28 (s, 1H), 8.08 (s, 1H), 7.66 (br s, 3H), 7.49 (t, |
| | dioxaborolan-2- | | J = 7.1 Hz, 1H), 7.41-7.36 (m, 1H), 7.29-7.19 (m, 3H), 6.65 |
| | yl)isoquinolin-1(2H)- | | (d, J = 7.2 Hz, 1H), 4.45 (br d, J = 14.0 Hz, 1H), 4.35 (br d, |
| | one | | J = 8.6 Hz, 1H), 3.76-3.71 (m, 1H), 3.19-3.04 (m, 2H), 2.98- |
| | | | 2.92 (m, 1H), 2.38-2.32 (m, 1H), 1.84-1.77 (m, 2H), 1.57- |
| | | | 1.53 (m, 1H) |
| 371 | (1,3,5-trimethyl-1H- | 2 | Method 2: m/z 447 (M + H)⁺, RT 1.02 min |
| | pyrazol-4-yl)boronic | | |
| | acid (commercial) | | |
| 372 | 7-(4,4,5,5-tetramethyl- | 2 | Method 2: m/z 485 (M + H)⁺, RT 1.04 min |
| | 1,3,2-dioxaborolan-2- | | |
| | yl)-3,4- | | |
| | dihydroquinazolin- | | |
| | 2(1H)-one | | |
| 373 | 4-chloro-3,3-difluoro- | 2 | (DMSO-d₆) δ 13.35 (s, 1H), 11.51 (br s, 1H), 8.36 (s, 1H), |
| | 5-(4,4,5,5-tetramethyl- | | 7.85 (d, J = 8.1 Hz, 1H), 7.57 (br s, 3H), 7.41 (t, J = 7.6 Hz, |
| | 1,3,2-dioxaborolan-2- | | 1H), 7.35-7.29 (m, 1H), 7.17-7.12 (m, 2H), 7.05 (d, J = 8.3 |
| | yl)-1-((2- | | Hz, 1H), 4.38 (br d, J = 14.3 Hz, 1H), 4.25 (br d, J = 9.7 Hz, |
| | (trimethylsilyl)ethoxy) | | 1H), 3.65 (dd, J = 14.3, 6.4 Hz, 1H), 3.12-2.97 (m, 2H), |
| | methyl)indolin-2-one | | 2.90-2.84 (m, 1H), 2.35-2.28 (m, 1H), 1.78-1.69 (m, 2H), |
| | | | 1.49-1.46 (m, 1H) |
| 374 | 2,3-difluoro-4- | 2 | (DMSO-d₆) δ 13.15 (br s, 1H), 10.50 (br s, 1H), 8.28 (s, |
| | (4,4,5,5-tetramethyl- | | 1H), 7.62 (br t, J = 7.2 Hz, 1H), 7.50 (br s, 3H), 7.33 (br t, |
| | 1,3,2-dioxaborolan-2- | | J = 7.0 Hz, 1H), 7.26-7.20 (m, 1H), 7.09-7.04 (m, 2H), 6.78 |
| | yl)phenol | | (d, J = 8.1 Hz, 1H), 4.30 (br d, J = 14.3 Hz, 1H), 4.18 (br d, |
| | | | J = 9.4 Hz, 1H), 3.57 (dd, J = 14.1, 6.5 Hz, 1H), 3.01-2.87 (m, |
| | | | 2H), 2.82-2.75 (m, 1H), 2.22-2.15 (m, 1H), 1.68-1.60 (m, |
| | | | 2H), 1.40-1.37 (m, 1H) |
| 375 | 6-chloro-5-(4,4,5,5- | 2 | Method 2: m/z 518 (M + H)⁺, RT 1.07 min |
| | tetramethyl-1,3,2- | | |
| | dioxaborolan-2- | | |
| | yl)indolin-2-one | | |
| 376 | 8-fluoranyl-5-(4,4,5,5- | 2 | (DMSO-d₆) δ 13.53 (br s, 1H), 9.31 (d, J = 8.33 Hz, 1H), |
| | tetramethyl-1,3,2- | | 9.03 (dd, J = 1.32, 3.95 Hz, 1H), 8.48 (s, 1H), 8.40-8.36 (m, |
| | dioxaborolan-2- | | 1H), 7.90-7.75 (m, 1H), 7.73-7.70 (m, 1H), 7.67 (br s, 3H), |
| | yl)quinoline | | 7.51-7.47 (m, 1H), 7.41-7.36 (m, 1H), 7.25-7.19 (m, 2H), |
| | (commercial) | | 4.47 (d, J = 14.03 Hz, 1H), 4.37-4.33 (m, 1H), 3.78-3.73 (m, |
| | | | 1H), 3.16-3.08 (m, 2H), 2.96-2.93 (m, 1H), 2.36-2.32 (m, |
| | | | 1H), 1.85-1.79 (m, 2H), 1.57-1.53 (m, 1H) |
| 381 | (2,6-difluoro-4- | 2 | (DMSO-d₆) δ 13.48 (s, 1H), 8.53 (s, 1H), 7.98 (d, J = 7.89 |
| | (4,4,5,5-tetramethyl- | | Hz, 2H), 7.65 (br s, 3H), 7.49 (t, J = 7.45 Hz, 1H), 7.41-7.36 |
| | 1,3,2-dioxaborolan-2- | | (m, 1H), 7.25-7.19 (m, 2H), 4.55 (s, 2H), 4.45 (d, J = 14.47 |
| | yl)phenyl)methanol | | Hz, 1H), 4.36-4.33 (m, 1H), 3.75 (dd, J = 14.47, 6.58 Hz, |
| | | | 1H), 3.16-3.04 (m, 3H), 3.0-2.93 (m, 1H), 2.39-2.34 (m, |
| | | | 1H), 1.84-1.80 (2H), 1.57-1.53 (m, 1H) |
| 389 | Intermediate 138 | 1 | (DMSO-d₆) δ 13.45 (s, 1H), 9.22 (s, 1H), 8.48 (s, 1H), 8.19 |
| | | | (t, J = 59.19 Hz, 1H), 8.15 (t, J = 8.77 Hz, 1H), 7.72 (d, J = |
| | | | 9.21 Hz, 1H), 7.66 (br s, 3H), 7.49 (t, J = 8.33 Hz 1H), 7.42- |
| | | | 7.36 (m, 1H), 7.25-7.20 (m, 2H), 4.47 (d, J = 14.47 Hz, 1H), |
| | | | 4.37-4.37 (m, 1H), 3.75 (dd, J = 14.47, 7.02 Hz, 1H), 3.18- |
| | | | 3.05 (m, 2H), 3.29-2.92 (m, 1H), 2.38-2.33 (m, 1H), 1.84- |
| | | | 1.80 (m, 2H), 1.57-1.53 (m, 1H) |
| 390 | (1-phenyl-1H-pyrazol- | 2 | Method 2: m/z 481 (M + H)⁺, RT 1.30 min |
| | 4-yl)boronic acid | | |
| | (commercial) | | |
| 391 | Intermediate 139 | 1 | (DMSO-d₆) δ 13.68 (s, 1H), 8.50 (s, 1H), 8.25-8.19 (m, |
| | | | 2H), 7.69 (br s, 3H), 7.50 (t, J = 7.02 Hz, 1H), 7.41-7.36 (m, |
| | | | 1H), 7.25-7.19 (m, 2H), 4.46 (d, J = 14.03 Hz, 1H), 4.36 (br |
| | | | d, J = 10.96 Hz, 1H), 3.76 (dd, J = 14.03, 6.14 Hz, 1H), 3.18- |
| | | | 3.07 (m, 2H), 3.0-2.94 (m, 1H), 2.39-2.32 (m, 1H), 1.84- |
| | | | 1.76 (m, 2H), 1.58-1.54 (m, 1H) |
| 392 | Intermediate 105 | 2 + | (DMSO-d₆) δ 13.40 (s, 1H), 12.36 (s, 1H), 8.43 (s, 1H), |
| | | 1:1 | 7.77 (d, J = 8.33 Hz, 1H), 7.66 (br s, 3H), 7.49 (t, J = 7.02 |
| | | MeOH:Ethylen | Hz, 1H), 7.41-7.36 (m, 1H), 7.26-7.19 (m, 3H), 4.45 (d, J = |
| | | diamine | 14.03 Hz, 1H), 4.33 (br d, J = 12.08 Hz, 1H), 3.72 (dd, |
| | | | J = 14.03, 6.14 Hz, 1H), 3.17-3.08 (m, 2H), 2.97-2.91 (m, |
| | | | 1H), 2.37-2.31 (m, 1H), 1.83-1.78 (m, 2H), 1.56-1.53 (m, |
| | | | 1H) |
| 393 | Intermediate 106 | 2 | (DMSO-d₆) δ 13.36 (s, 1H), 8.57 (s, 1H), 8.43 (s, 1H), 7.71- |
| | | | 7.64 (m, 5H), 7.48 (t, J = 7.89 Hz, 1H), 7.41-7.35 (m, 1H), |
| | | | 7.25-7.21 (m, 2H), 4.66 (br s, 2H), 4.46 (d, J = 13.59 Hz, |
| | | | 1H), 4.37-4.32 (m, 1H), 3.89-3.86 (m, 2H), 3.71 (dd, |
| | | | J = 14.03, 5.7 Hz, 1H), 3.25 (s, 3H), 3.17-3.11 (m, 2H), 2.98- |
| | | | 2.90 (m, 1H), 2.37-2.31 (m, 1H), 1.83-1.75 (m, 2H), 1.56- |
| | | | 1.52 (m, 1H) |
| 399 | (3-fluoro-4- | 2 | Method 2: m/z 463 (M + H)⁺, RT min 1.32 |
| | methoxyphenyl)boronic | | |
| | acid | | |
| 413 | Intermediate 180b | 2 | (DMSO-d₆) δ 13.36 (m, 1H), 8.43 (s, 1H), 8.15 (s, 1H), 7.81 |
| | | | (s, 2H), 7.65 (br s, 3H), 7.48 (br d, J = 1.8 Hz, 1H), 7.42- |
| | | | 7.33 (m, 1H), 7.27-7.15 (m, 2H), 4.46 (br d, J = 14.3 Hz, |
| | | | 1H), 4.39-4.29 (m, 1H), 3.94-3.83 (m, 1H), 3.71 (m, 1H), |
| | | | 3.21-3.05 (m, 2H), 3.01-2.82 (m, 1H), 2.43-2.26 (m, 1H), |
| | | | 1.87-1.73 (m, 2H), 1.62-1.50 (m, 1H), 1.23-1.10 (m, 4H) |
| 417 & | Intermediate 107 | 2 | 417 Method 3: m/z 505 (M + H)⁺ (first eluted), RT 1.9 min; |
| 418 | | | 418 Method 3: m/z 505 (M + H)⁺ (second eluted), RT 1.34 |
| | | | min |
| 420 | Intermediate 180a | 2 | (DMSO-d₆) δ 13.37 (br s, 1H), 8.69 (s, 1H), 8.43 (s, 1H), |
| | | | 7.66 (br d, J = 3.7 Hz, 5H), 7.54-7.45 (m, 1H), 7.42-7.32 (m, |
| | | | 1H), 7.28-7.15 (m, 2H), 4.46 (br d, J = 15.1 Hz, 1H), 4.41- |
| | | | 4.31 (m, 1H), 4.29-4.21 (m, 1H), 3.76-3.68 (m, 1H), 3.21- |
| | | | 3.01 (m, 2H), 2.99-2.88 (m, 1H), 2.40-2.28 (m, 1H), 1.88- |
| | | | 1.73 (m, 2H), 1.55 (br dd, J = 6.8, 8.1 Hz, 1H), 1.42-1.32 (m, |
| | | | 2H), 1.21-1.11 (m, 2H) |
| 428 | Intermediate 181a | 2 | (DMSO-d₆) δ 13.37 (s, 1H), 8.49 (s, 1H), 8.43 (s, 1H), 7.72- |
| | | | 7.57 (m, 5H), 7.53-7.45 (m, 1H), 7.42-7.34 (m, 1H), 7.27- |
| | | | 7.17 (m, 2H), 5.53 (s, 2H), 4.47 (dd, J = 1.2, 15.2 Hz, 1H), |
| | | | 4.39-4.30 (m, 1H), 3.76-3.66 (m, 1H), 3.11 (s, 3H), 3.07- |
| | | | 2.93 (m, 3H), 2.89 (s, 3H), 2.36-2.31 (m, 1H), 1.86-1.75 |
| | | | (m, 2H), 1.57-1.51 (m, 1H) |
| 432 | Intermediate 181b | 2 | (DMSO-d₆) δ 13.37 (br s, 1H), 8.43 (s, 1H), 8.20 (s, 1H), |
| | | | 7.81-7.57 (m, 5H), 7.53-7.45 (m, 1H), 7.42-7.32 (m, 1H), |
| | | | 7.26-7.16 (m, 2H), 5.52 (s, 2H), 4.47 (br d, J = 14.9 Hz, 1H), |
| | | | 4.40-4.29 (m, 1H), 3.77-3.66 (m, 1H), 3.20-3.04 (m, 5H), |
| | | | 3.00-2.90 (m, 1H), 2.86 (s, 3H), 2.39-2.27 (m, 1H), 1.88- |
| | | | 1.72 (m, 2H), 1.60-1.48 (m, 1H) |
| 438 | Intermediate 113 | 1 | (DMSO-d₆ + TFA) δ 8.84 (dd, J = 0.6, 7.2 Hz, 1H), 8.49 (s, |
| | | | 1H), 8.15 (d, J = 2.2 Hz, 1H), 7.68 (br s, 3H), 7.56-7.44 (m, |
| | | | 2H), 7.43-7.32 (m, 1H), 7.29-7.11 (m, 2H), 6.88-6.76 (m, |
| | | | 1H), 4.47 (d, J = 14.6 Hz, 1H), 4.40-4.29 (m, 1H), 3.74 (dd, |
| | | | J = 6.4, 14.4 Hz, 1H), 3.24-3.03 (m, 2H), 3.02-2.88 (m, 1H), |
| | | | 2.42-2.30 (m, 1H), 1.90-1.68 (m, 2H), 1.61-1.48 (m, 1H) |
| 500 | Intermediate 141 | 1 | (DMSO-d₆ + TFA) δ 8.47 (s, 1H), 8.24 (d, J = 5.1 Hz, 1H), |
| | | | 7.67 (br s, 3H), 7.51 (br d, J = 5.1 Hz, 2H), 7.43-7.32 (m, |
| | | | 1H), 7.27-7.14 (m, 2H), 4.46 (br d, J = 14.3 Hz, 1H), 4.40- |
| | | | 4.28 (m, 1H), 4.01 (s, 3H), 3.73 (dd, J = 6.4, 14.3 Hz, 1H), |
| | | | 3.23-2.86 (m, 3H), 2.42-2.29 (m, 1H), 1.88-1.70 (m, 2H), |
| | | | 1.62-1.47 (m, 1H) |
| 509 | (3-chloro-2- | 1 | (DMSO-d₆ + TFA) δ 8.50 (s, 1H), 8.30 (d, J = 4.9 Hz, 1H), |
| | fluoropyridin-4- | | 8.07 (d, J = 5.1 Hz, 1H), 7.66 (br s, 3H), 7.44-7.53 (m, 1H), |
| | yl)boronic acid | | 7.31-7.41 (m, 1H), 7.23-7.13 (m, 2H), 4.51-4.29 (m, 2H), |
| | | | 3.74 (dd, J = 14.4, 6.4 Hz, 1H), 3.17-2.90 (m, 3H), 2.42-2.33 |
| | | | (m, 1H), 1.87-1.76 (m, 2H), 1.59-1.50 (m, 1H) |
| 521 | Intermediate 61 | 1 | (DMSO-d₆ + TFA) δ 9.45 (dd, J = 1.7, 8.8 Hz, 1H), 9.17 (dd, |
| | | | J = 1.7, 4.2 Hz, 1H), 8.66-8.44 (m, 3H), 7.80 (dd, J = 4.2, 8.8 |
| | | | Hz, 1H), 7.67 (br s, 3H), 7.54-7.44 (m, 1H), 7.43-7.32 (m, |
| | | | 1H), 7.29-7.12 (m, 2H), 4.50 (br d, J = 14.2 Hz, 1H), 4.43- |
| | | | 4.28 (m, 1H), 3.78 (dd, J = 6.3, 14.3 Hz, 1H), 3.66 (s, 3H), |
| | | | 3.24-2.89 (m, 3H), 2.44-2.30 (m, 1H), 1.90-1.71 (m, 2H), |
| | | | 1.66-1.46 (m, 1H) |

### Example 106: ((1S,6R,7R)-7-(2,6-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

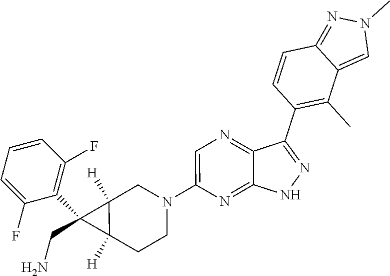 [SMILES: Cc1c(-c2n[nH]c3nc(N4CC[C@@H]5[C@H](C4)[C@@]5(CN)c4c(F)cccc4F)cnc23)ccc2nn(C)cc12]

The above example was synthesized using the procedure reported for Example 2 using Intermediate 52 for step 1 and Intermediate 53 for step 2; Example 106 was obtained as a white powder (TFA salt; 4.0 mg, 38%). ¹H NMR (400 MHz, DMSO-d₆) δ 13.17 (br s, 1H), 8.50 (s, 1H), 8.44 (s, 1H), 7.76-7.72 (br in, 4H), 7.50 (d, J=8.2 Hz, 1H), 7.47-7.40 (m, 1H), 7.12 (t, J=8.3 Hz, 2H), 4.49 (br d, J=14.0 Hz, 1H), 4.36 (br d, J=8.3 Hz, 1H), 4.19 (s, 3H), 3.75-3.70 (m, 1H), 3.17-3.04 (m, 2H), 2.96-2.85 (m, 1H), 2.64 (s, 3H), 2.39-2.28 (m, 1H), 1.91-1.79 (m, 2H), 1.62-1.55 (m, 1H); LCMS (ES⁺) Method 2: m/z 501 (M+H)⁺, RT 1.15 min.

### Example 107: ((1S,6R,7S)-3-(3-(7-chloro-2-methylbenzo[d]thiazol-6-yl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

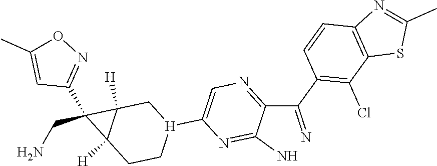

The above example was synthesized using the procedure reported for Example 2 using Intermediates 18, 44, and Intermediate 54; Example 107 was obtained as a white powder (TFA salt; 7.5 mg, 49%). ¹H NMR (400 MHz, DMSO-d₆) δ 13.49 (br s, 1H), 8.42 (s, 1H), 8.02 (d, J=8.3 Hz, 1H), 7.96 (d, J=8.3 Hz, 1H), 7.87 (br s, 3H), 6.14 (s, 1H), 4.32 (br d, J=14.5 Hz, 2H), 3.73 (br dd, J=14.4, 6.3 Hz, 1H), 3.43-3.38 (m, 2H), 3.24-3.19 (m, 1H), 3.01-2.92 (m, 1H), 2.87 (s, 3H), 2.38 (s, 3H), 2.35-2.27 (m, 1H), 1.99-1.92 (m, 1H), 1.80-1.72 (m, 1H); LCMS (ES⁺) Method 2: m/z 507 (M+H)⁺, RT 1.25 min.

The following examples were synthesized using the above procedure with Intermediates 18, 44 and the appropriate boronic ester or acid.

| Example # | Boronic ester or acid | ¹H NMR (400 MHZ) |
|---|---|---|
| 108 | Intermediate 55 | (DMSO-d₆) δ 13.32 (br s, 1H), 8.37 (s, 1H), 7.87 (br s, 3H), |
| | | 7.57 (d, J = 8.8 Hz, 1H), 7.45 (d, J = 8.8 Hz, 1H), 6.14 (s, 1H), |
| | | 4.31 (br d, J = 14.0 Hz, 2H), 4.11 (s, 3H), 3.71 (br dd, J = 14.7, |
| | | 6.4 Hz, 1H), 3.42-3.40 (m, 1H), 3.26-3.20 (m, 1H), 2.98-2.90 |
| | | (m, 1H), 2.86 (s, 3H), 2.39 (s, 3H), 2.35-2.27 (m, 1H), 1.97- |
| | | 1.90 (m, 1H), 1.80-1.72 (m, 2H) |
| 109 | Intermediate 56 | (DMSO-d₆) δ 13.36 (br s, 1H), 8.39 (s, 1H), 7.87 (br s, 3H), |
| | | 7.52 (br s, 2H), 6.14 (s, 1H), 4.31 (br d, J = 14.0 Hz, 2H), 4.10 |
| | | (s, 3H), 4.02 (s, 3H), 3.72 (br dd, J = 14.5, 6.4 Hz, 1H), 3.5 (m, |
| | | 1H), 3.25-3.20 (m, 1H), 2.98-2.92 (m, 1H), 2.39 (s, 3H), 2.33- |
| | | 2.28 (m, 1H), 1.98-1.92 (m, 1H), 1.81-1.72 (m, 2H) |
| 112 | Intermediate 58 | (DMSO-d₆) δ 13.56 (br s, 1H), 8.74 (s, 1H), 8.42 (s, 1H), 8.11 |
| | | (d, J = 8.6 Hz, 1H), 8.00 (d, J = 8.6 Hz, 1H), 7.87 (br s, 3H), 6.15 |
| | | (s, 1H), 4.33 (br d, J = 13.4 Hz, 2H), 4.13 (s, 3H), 3.74 (br dd, |
| | | J = 14.7, 6.4 Hz, 1H), 3.25-3.20 (m, 2H), 3.02-2.93 (m, 1H), |
| | | 2.39 (s, 3H), 2.35-2.29 (m, 1H), 1.99-1.92 (m, 1H), 1.79-1.73 |
| | | (m, 2H) |
| 113 | 3-(4,4,5,5-tetramethyl- | (DMSO-d₆) δ 13.38 (s, 1H), 9.17 (d, J = 8.3 Hz, 1H), 8.79 (s, |
| | 1,3,2-dioxaborolan-2- | 1H), 8.66 (d, J = 3.1 Hz, 1H), 8.48 (s, 1H), 7.87 (br s, 3H), 7.58 |
| | yl)thieno[2,3-b]pyridine | (br dd, J = 8.1, 4.4 Hz, 1H), 6.15 (s, 1H), 4.34 (br d, J = 14.7 Hz, |
| | | 2H), 3.77 (dd, J = 14.4, 6.3 Hz, 1H), 3.5 (m, 1H), 3.23-3.18 (m, |
| | | 1H), 3.01-2.96 (m, 1H), 2.39 (s, 3H), 2.35-2.29 (m, 1H), 1.99- |
| | | 1.94 (m, 1H), 1.80-1.73 (m, 2H) |
| 114 | Intermediate 60 | (DMSO-d₆) δ 13.82 (br s, 1H), 9.10 (d, J = 4.4 Hz, 1H), 8.87- |
| | | 8.85 (m, 1H), 8.51 (s, 1H), 8.45 (d, J = 4.6 Hz, 1H), 7.88 (br s, |
| | | 3H), 7.67-7.64 (m, 2H), 6.15 (s, 1H), 4.34 (br d, J = 13.8 Hz, |
| | | 2H), 3.77 (dd, J = 14.4, 6.4 Hz, 1H), 3.44-3.39 (m, 1H), 3.23- |
| | | 3.18 (m, 1H), 3.02-2.96 (m, 1H), 2.38 (s, 3H), 2.35-2.29 (m, |
| | | 1H), 1.99-1.93 (m, 1H), 1.80-1.73 (m, 2H) |
| 115 | 6-methoxy-5-(4,4,5,5- | (DMSO-d₆) δ 13.51 (br s, 1H), 8.95 (d, J = 4.2 Hz, 1H), 8.34- |
| | tetramethyl-1,3,2- | 8.32 (m, 2H), 8.22-8.12 (m, 1H), 8.01-7.96 (m, 4H), 7.65-7.57 |
| | dioxaborolan-2- | (m, 1H), 6.15 (s, 1H), 4.30 (br d, J = 14.3 Hz, 2H), 3.87 (s, 3H), |
| | yl)quinoline | 3.74-3.68 (m, 1H), 3.5 (m, 1H), 3.28-3.24 (m, 1H), 2.98-2.92 |
| | | (m, 1H), 2.38 (s, 3H), 2.34-2.29 (m, 1H), 1.97-1.92 (m, 1H), |
| | | 1.78-1.72 (m, 2H) |
| 116 | Intermediate 62 | (DMSO-d₆) δ 13.51 (s, 1H), 8.75 (d, J = 6.8 Hz, 1H), 8.62 (d, |
| | | J = 7.0 Hz, 1H), 8.54 (s, 1H), 8.11 (d, J = 2.2 Hz, 1H), 7.73 (br |
| | | s, 3H), 7.46-7.41 (m, 1H), 7.39 (br d, J = 1.5 Hz, 1H), 7.15-7.07 |
| | | (m, 3H), 4.49 (br d, J = 13.8 Hz, 1H), 4.38 (br d, J = 13.6 Hz, |
| | | 1H), 3.80-3.75 (m, 1H), 3.16-3.02 (m, 2H), 2.97-2.91 (m, 1H), |
| | | 2.38-2.33 (m, 1H), 1.90-1.81 (m, 2H), 1.61-1.58 (m, 1H) |
| 125 | (8-methoxyquinolin-4- | (DMSO-d₆) δ 14.21 (br s, 1H), 9.16 (d, J = 5.3 Hz, 1H), 8.92 (br |
| | yl)boronic acid | d, J = 8.1 Hz, 1H), 8.85 (br s, 1H), 8.60 (s, 1H), 8.07 (br s, 3H), |
| | | 7.83 (t, J = 8.2 Hz, 1H), 7.58 (br d, J = 7.9 Hz, 1H), 6.18 (s, 1H), |
| | | 4.39-4.33 (m, 2H), 4.13 (s, 3H), 3.82 (br dd, J = 14.5, 6.4 Hz, |
| | | 1H), 3.42-3.37 (m, 1H), 3.24-3.19 (m, 1H), 3.06-2.99 (m, 1H), |
| | | 2.39 (s, 3H), 2.36-2.29 (m, 1H), 1.98-1.92 (m, 1H), 1.85-1.74 |
| | | (m, 2H) |
| 126 | (8- | (DMSO-d₆) δ 13.64 (br s, 1H), 9.32 (d, J = 8.8 Hz, 1H), 9.09 |
| | (trifluoromethoxy) | (dd, J = 4.1, 1.6 Hz, 1H), 8.47 (s, 1H), 8.42 (d, J = 8.1 Hz, 1H), |
| | quinolin-5-yl)boronic acid | 7.99 (dd, J = 8.1, 1.1 Hz, 1H), 7.88 (br s, 3H), 7.74 (dd, J = 8.8, |
| | (commercial) | 4.2 Hz, 1H), 6.15 (s, 1H), 4.34 (br d, J = 13.8 Hz, 2H), 3.79- |
| | | 3.73 (m, 1H), 3.44-3.39 (m, 1H), 3.24-3.19 (m, 1H), 3.01-2.96 |
| | | (m, 1H), 2.38 (s, 3H), 2.35-2.29 (m, 1H), 1.99-1.93 (m, 1H), |
| | | 1.80-1.72 (m, 2H) |
| 127 | Intermediate 53 | (DMSO-d₆) δ 13.23 (br s, 1H), 8.50 (s, 1H), 8.39 (s, 1H), 7.87 |
| | | (br s, 3H), 7.72 (br d, J = 8.8 Hz, 1H), 7.50 (br d, J = 9.2 Hz, 1H), |
| | | 6.15 (s, 1H), 4.32 (br d, J = 13.8 Hz, 2H), 4.19 (s, 3H), 3.88 (m, |
| | | 1H), 3.46-3.40 (m, 1H), 3.30-3.19 (m, 1H), 2.97-2.91 (m, 1H), |
| | | 2.64 (s, 3H), 2.39 (s, 3H), 2.34-2.28 (m, 1H), 1.99-1.92 (m, |
| | | 1H), 1.79-1.72 (m, 2H) |
| 128 | Intermediate 66 | (DMSO-d₆) δ 13.53 (br s, 1H), 9.25 (br d, J = 8.1 Hz, 1H), 8.97 |
| | | (dd, J = 4.1, 1.6 Hz, 1H), 8.46 (s, 1H), 8.36 (d, J = 7.5 Hz, 1H), |
| | | 7.92 (d, J = 7.5 Hz, 1H), 7.87 (br s, 3H), 7.64-7.61 (m, 1H), 6.15 |
| | | (s, 1H), 5.16 (s, 2H), 4.35 (br d, J = 14.5 Hz, 2H), 3.78-3.73 (m, |
| | | 1H), 3.50 (s, 3H), 3.45-3.39 (m, 1H), 3.24-3.19 (m, 1H), 3.02- |
| | | 2.94 (m, 1H), 2.39 (s, 3H), 2.35-2.29 (m, 1H), 1.98-1.91 (m, |
| | | 1H), 1.80-1.72 (m, 2H) |
| 129 | Intermediate 62 | (DMSO-d₆) δ 13.52 (s, 1H), 8.75 (d, J = 6.8 Hz, 1H), 8.62 (d, |
| | | J = 7.2 Hz, 1H), 8.48 (s, 1H), 8.11 (d, J = 2.2 Hz, 1H), 7.87 (br |
| | | s, 3H), 7.38 (d, J = 1.3 Hz, 1H), 7.09 (d, J = 7.0 Hz, 1H), 6.15 (s, |
| | | 1H), 4.34 (br d, J = 14.5 Hz, 2H), 3.79-3.73 (m, 1H), 3.44-3.39 |
| | | (m, 1H), 3.23-3.18 (m, 1H), 3.02-2.94 (m, 1H), 2.39 (s, 3H), |
| | | 2.35-2.29 (m, 1H), 1.98-1.91 (m, 1H), 1.80-1.73 (m, 2H) |
| 130 | 7-fluoro-5-(4,4,5,5- | (DMSO-d₆) δ 13.69 (s, 1H), 9.39 (br d, J = 8.8 Hz, 1H), 8.99 |
| | tetramethyl-1,3,2- | (dd, J = 4.2, 1.5 Hz, 1H), 8.51 (s, 1H), 8.38 (dd, J = 10.0, 2.5 Hz, |
| | dioxaborolan-2- | 1H), 7.91-7.83 (m, 4H), 7.61 (dd, J = 8.8, 4.2 Hz, 1H), 6.15 (s, |
| | yl)quinoline | 1H), 4.34 (br d, J = 14.3 Hz, 2H), 3.77 (br dd, J = 14.5, 6.4 Hz, |
| | | 1H), 3.43-3.39 (m, 1H), 3.23-3.18 (m, 1H), 3.03-2.95 (m, 1H), |
| | | 2.39 (s, 3H), 2.36-2.28 (m, 1H), 1.99-1.93 (m, 1H), 1.80-1.73 |
| | | (m, 2H) |
| 133 | Intermediate 68 | DMSO-d₆) δ 13.68 (br s, 1H), 9.36 (d, J = 8.8 Hz, 1H), 9.06 (dd, |
| | | J = 4.1, 1.6 Hz, 1H), 8.52 (d, J = 7.5 Hz, 1H), 8.49 (s, 1H), 8.21 |
| | | (d, J = 7.7 Hz, 1H), 7.96 (bs, 1H) 7.89 (br s, 3H), 7.73 (dd, |
| | | J = 8.7, 4.1 Hz, 1H), 6.15 (s, 1H), 4.35 (br d, J = 14.3 Hz, 2H), |
| | | 3.77 (br dd, J = 14.7, 6.6 Hz, 1H), 3.44-3.39 (m, 1H), 3.24-3.18 |
| | | (m, 1H), 3.03-2.96 (m, 1H), 2.39 (s, 3H), 2.36-2.30 (m, 1H), |
| | | 1.99-1.94 (m, 1H), 1.81-1.75 (m, 2H) |
| 134 | Intermediate 69 | (DMSO-d₆) δ 13.51 (br s, 1H), 9.19 (br d, J = 8.3 Hz, 1H), 8.97 |
| | | (dd, J = 4.0, 1.8 Hz, 1H), 8.45 (s, 1H), 8.32 (d, J = 7.7 Hz, 1H), |
| | | 8.03 (d, J = 7.5 Hz, 1H), 7.89 (br s, 3H), 7.59 (dd, J = 8.6, 4.2 |
| | | Hz, 1H), 6.16 (s, 1H), 5.92 (q, J = 6.4 Hz, 1H), 4.35 (br d, |
| | | J = 14.3 Hz, 2H), 3.77 (br dd, J = 14.7, 6.6 Hz, 1H), 3.45-3.40 |
| | | (m, 1H), 3.26-3.19 (m, 1H), 3.02-2.94 (m, 1H), 2.39 (s, 3H), |
| | | 2.35-2.29 (m, 1H), 1.99-1.94 (m, 1H), 1.80-1.73 (m, 2H), 1.51 |
| | | (d, J = 6.4 Hz, 3H) |

### Example 110: (3-(3-(7-chloro-2-methylbenzo[d]thiazol-6-yl)-5-methyl-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

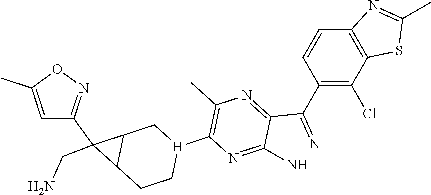

The above example was synthesized using the procedure reported for Example 2 using Intermediates 12, 57, and 54; Example 110 was obtained as a yellow powder (TFA salt; 9.0 mg, 51%). ¹H NMR (400 MHz, DMSO-d₆) δ 13.81 (br s, 1H), 8.04 (d, J=8.3 Hz, 1H), 7.94 (d, J=8.3 Hz, 1H), 7.91 (br s, 3H), 6.14 (s, 1H), 3.81-3.65 (m, 4H), 3.60-3.54 (m, 1H), 2.88 (s, 3H), 2.64-2.61 (m, 1H), 2.58 (s, 3H), 2.39 (s, 3H), 2.33-2.26 (m, 1H), 2.10-2.04 (m, 1H), 1.95-1.87 (m, 1H), 1.81-1.75 (m, 1H); LCMS (ES⁺) Method 2: m/z 521 (M+H)⁺, RT 1.36 min.

### Example 111: ((1S,6R,7S)-3-(3-(2-chloro-3-(oxazol-2-yl)phenyl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

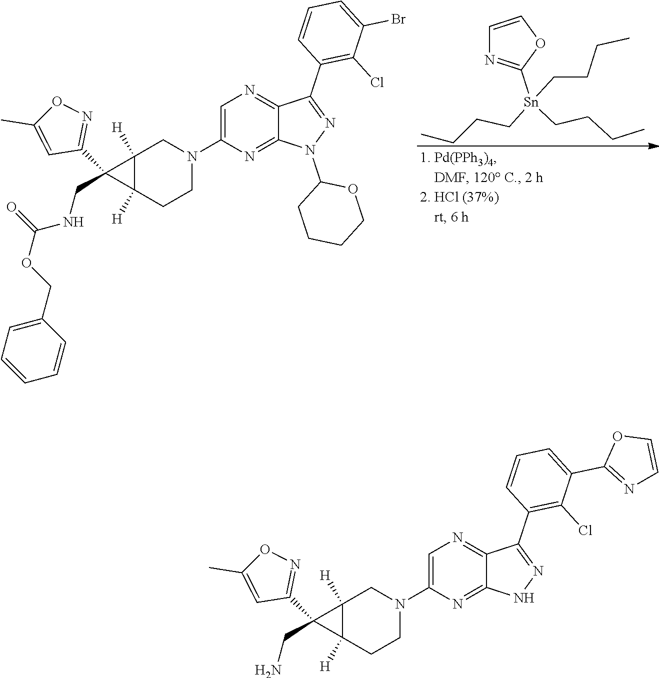 [SMILES: Cc1cc([C@@]2(CNC(=O)OCc3ccccc3)[C@@H]3CCN(c4cnc5c(-c6cccc(Br)c6Cl)nn(C6CCCCO6)c5n4)C[C@@H]32)no1 ; CCC[CH2][Sn]([CH2]CCC)([CH2]CCC)[c]1ncco1 ; Cc1cc([C@@]2(CN)[C@@H]3CCN(c4cnc5c(-c6cccc(-c7ncco7)c6Cl)n[nH]c5n4)C[C@@H]32)no1]

A solution of benzyl (((1S,6R,7S)-3-(3-(3-bromo-2-chlorophenyl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (prepared using the procedure reported for Example 2 step 1 and step 2 using Intermediates 18 and 44; 25 mg, 0.03 mmol), 2-(tributylstannyl)oxazole (13.6 uL, 0.04 mmol) and Pd(PPh3)₄ (3.9 mg, 0.003 mmol) in degassed DMF (0.8 mL) was heated at 120° C. for 2 h. After cooling mixture was diluted with EtOAc (40 mL), washed with brine, dried over Na₂SO₄, filtered, and concentrated in vacuo. The crude residue was purified by flash chromatography (0-100% EtOAc in petroleum ether) to afford the title compound as a yellow solid (6 mg, 0.01 mmol, 24%); LCMS (ES⁺) Method 2: m/z 721 (M+H)⁺, RT 2.24 min) which was treated with conc. HCl (37%; 0.4 mL, 0.04 mmol). The mixture was stirred at rt for 6 h. After volatiles removal in vacuo the residue was purified by preparative HPLC to obtain the title compound as a white solid (TFA salt; 2 mg, 48%). ¹H NMR (400 MHz, DMSO-d₆+TFA) δ 8.38 (s, 1H), 8.31 (s, 1H), 7.97 (d, J=8.1 Hz, 1H), 7.87 (br s, 3H), 7.80 (d, J=8.2 Hz, 1H), 7.63-7.58 (m, 1H), 7.46 (s, 1H), 6.11 (s, 1H), 4.32 (br d, J=14.3 Hz, 2H), 3.74-3.67 (m, 1H), 3.45-3.39 (m, 1H), 3.26-3.20 (m, 1H), 2.98-2.90 (m, 1H), 2.37 (s, 3H), 2.35-2.24 (m, 1H), 1.98-1.90 (m, 1H), 1.82-1.70 (m, 2H); LCMS (ES⁺) Method 2: m/z 503 (M+H)⁺, RT 1.17 min.

### Example 116: ((1S,6R,7R)-7-(2,6-difluorophenyl)-3-(3-(pyrazolo[1,5-a]pyridin-4-yl)-1H-pyrazolo[3,4-b]pyrazin-6-yl)-3-azabicyclo[4.1.0]heptan-7-yl)methanamine (Trifluoro Acetate Salt)

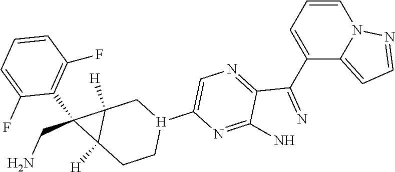

The above example was synthesized using the procedure reported for Example 2 using Intermediates 52, 44, and 62; Example 116 was obtained as a white powder (TFA salt; 3.5 mg, 24%). ¹H NMR (400 MHz, DMSO-d₆) δ 13.51 (s, 1H), 8.75 (d, J=6.8 Hz, 1H), 8.62 (d, J=7.0 Hz, 1H), 8.54 (s, 1H), 8.11 (d, J=2.2 Hz, 1H), 7.73 (br s, 3H), 7.46-7.41 (m, 1H), 7.39 (br d, J=1.5 Hz, 1H), 7.15-7.07 (m, 3H), 4.49 (br d, J=13.8 Hz, 1H), 4.38 (br d, J=13.6 Hz, 1H), 3.80-3.75 (m, 1H), 3.16-3.02 (m, 2H), 2.97-2.91 (m, 1H), 2.38-2.33 (m, 1H), 1.90-1.81 (m, 2H), 1.61-1.58 (m, 1H); LCMS (ES⁺) Method 2: m/z 473 (M+H)⁺, RT 1.30 min.

### Example 117: ((1S,6R,7R)-3-(3-(4-chloro-2-methyl-2H-indazol-5-yl)-1H-pyrazolo[3,4-b]pyrazin-6-yl)-7-(2,6-difluorophenyl)-3-azabicyclo[4.1.0]heptan-7-yl)methanamine (Trifluoro Acetate Salt)

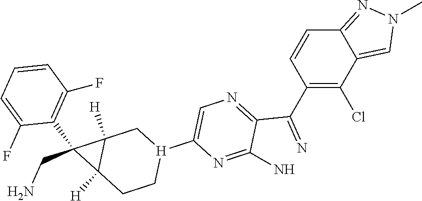

The above example was synthesized using the procedure reported for Example 2 using Intermediates 52, 44, and 45; Example 117 was obtained as a white powder (TFA salt; 2.5 mg, 20%). ¹H NMR (400 MHz, DMSO-d₆) δ 13.38 (br s, 1H), 8.57 (s, 1H), 8.46 (s, 1H), 7.73 (br s, 3H), 7.70-7.63 (m, 2H), 7.44 (br t, J=7.6 Hz, 1H), 7.13 (br t, J=8.4 Hz, 2H), 4.49 (br d, J=14.0 Hz, 1H), 4.37 (br d, J=12.7 Hz, 1H), 4.24 (s, 3H), 3.74 (br dd, J=14.8, 6.3 Hz, 1H), 3.18-3.03 (m, 2H), 2.96-2.87 (m, 1H), 2.39-2.30 (m, 1H), 1.91-1.79 (m, 2H), 1.62-1.55 (m, 1H); LCMS (ES⁺) Method 2: m/z 521 (M+H)⁺, RT 1.12 min.

### Example 120: ((1S,6R,7S)-3-(3-(4-chloro-2-methyl-2H-indazol-5-yl)-1H-pyrazolo[3,4-b]pyrazin-6-yl)-7-(5-fluoropyridin-2-yl)-3-azabicyclo[4.1.0]heptan-7-yl)methanamine (Trifluoro Acetate Salt)

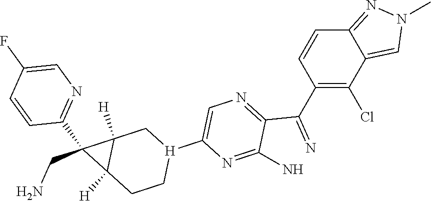

The above example was synthesized using the procedure reported for Example 2 using Intermediates 63, 44, and 45; Example 120 was obtained as a yellow powder (TFA salt; 7.0 mg, 88%). ¹H NMR (400 MHz, DMSO-d₆) δ 13.36 (br s, 1H), 8.56 (s, 1H), 8.49 (d, J=2.6 Hz, 1H), 8.41 (s, 1H), 7.79 (br s, 3H), 7.77-7.70 (m, 1H), 7.69-7.63 (m, 2H), 7.32 (dd, J=9.0, 4.2 Hz, 1H), 4.40-4.32 (m, 2H), 4.23 (s, 3H), 3.77-3.72 (m, 1H), 3.34-3.28 (m, 2H), 3.01-2.93 (m, 1H), 2.37-2.30 (m, 1H), 2.08-2.02 (m, 1H), 1.83-1.75 (m, 2H); LCMS (ES⁺) Method 2: m/z 504 (M+H)⁺, RT 1.02 min.

### Example 121: ((1S,6R,7S)-3-(3-(7-chloro-2-methylbenzo[d]thiazol-6-yl)-1H-pyrazolo[3,4-b]pyrazin-6-yl)-7-(5-fluoropyridin-2-yl)-3-azabicyclo[4.1.0]heptan-7-yl)methanamine (Trifluoro Acetate Salt)

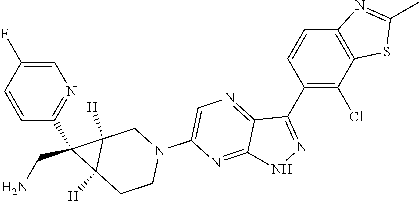 [SMILES: Cc1nc2ccc(-c3n[nH]c4nc(N5CC[C@@H]6[C@H](C5)[C@@]6(CN)c5ccc(F)cn5)cnc34)c(Cl)c2s1]

The above example was synthesized using the procedure reported for Example 2 using Intermediates 63, 44, and 54; Example 121 was obtained as a yellow powder (TFA salt; 3.2 mg, 26%). ¹H NMR (400 MHz, DMSO-d₆) δ 13.48 (br s, 1H), 8.50 (d, J=2.4 Hz, 1H), 8.43 (s, 1H), 8.03 (d, J=8.3 Hz, 1H), 7.97 (d, J=8.3 Hz, 1H), 7.79 (br s, 3H), 7.77-7.74 (m, 1H), 7.32 (dd, J=8.9, 4.1 Hz, 1H), 4.40-4.32 (m, 2H), 3.75 (br dd, J=14.5, 6.4 Hz, 1H), 3.57-3.50 (m, 2H), 3.02-2.94 (m, 1H), 2.87 (s, 3H), 2.37-2.31 (m, 1H), 2.08-2.02 (m, 1H), 1.84-1.75 (m, 2H); LCMS (ES⁺) Method 2: m/z 521 (M+H)⁺, RT 1.19 min.

### Example 123: ((1S,6R,7S)-3-(3-(4-chloro-2-methyl-2H-indazol-5-yl)-1H-pyrazolo[3,4-b]pyrazin-6-yl)-7-(isoxazol-3-yl)-3-azabicyclo[4.1.0]heptan-7-yl)methanamine (Trifluoro Acetate Salt)

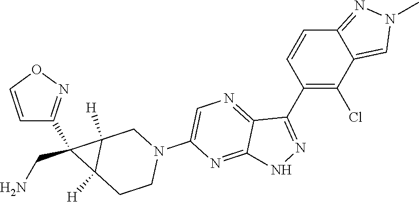 [SMILES: Cn1cc2c(Cl)c(-c3n[nH]c4nc(N5CC[C@@H]6[C@H](C5)[C@@]6(CN)c5ccon5)cnc34)ccc2n1]

The above example was synthesized using the procedure reported for Example 2 using Intermediates 64, 44, and 45; Example 123 was obtained as a yellow powder (TFA salt; 6.5 mg, 36%). ¹H NMR (400 MHz, DMSO-d₆) δ 13.37 (br s, 1H), 8.90 (d, J=1.3 Hz, 1H), 8.56 (s, 1H), 8.40 (s, 1H), 7.89 (br s, 3H), 7.69-7.63 (m, 2H), 6.52 (s, 1H), 4.33 (br d, J=14.3 Hz, 2H), 4.23 (s, 3H), 3.74 (br dd, J=14.5, 6.6 Hz, 1H), 3.50-3.44 (m, 1H), 3.31-3.25 (m, 1H), 3.01-2.93 (m, 1H), 2.38-2.27 (m, 1H), 2.03-1.98 (m, 1H), 1.84-1.75 (m, 2H); LCMS (ES⁺) Method 2: m/z 476 (M+H)⁺, RT 0.97 min.

### Example 124: ((1S,6R,7S)-3-(3-(7-chloro-2-methylbenzo[d]thiazol-6-yl)-1H-pyrazolo[3,4-b]pyrazin-6-yl)-7-(isoxazol-3-yl)-3-azabicyclo[4.1.0]heptan-7-yl)methanamine (Trifluoro Acetate Salt)

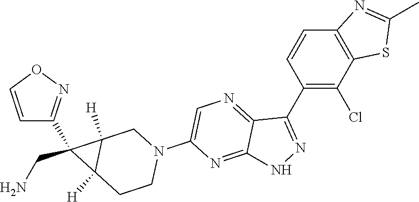 [SMILES: Cc1nc2ccc(-c3n[nH]c4nc(N5CC[C@@H]6[C@H](C5)[C@@]6(CN)c5ccon5)cnc34)c(Cl)c2s1]

The above example was synthesized using the procedure reported for Example 2 using Intermediates 64, 44, and 54; Example 124 was obtained as a yellow powder (TFA salt; 9.2 mg, 25%). ¹H NMR (400 MHz, DMSO-d₆) δ 13.50 (br s, 1H), 8.90 (d, J=1.5 Hz, 1H), 8.42 (s, 1H), 8.03 (d, J=8.3 Hz, 1H), 7.97 (d, J=8.3 Hz, 1H), 7.89 (br s, 3H), 6.52 (d, J=1.3 Hz, 1H), 4.33 (br d, J=14.0 Hz, 2H), 3.75 (br dd, J=14.7, 6.4 Hz, 1H), 3.49-3.44 (m, 1H), 3.29-3.24 (m, 1H), 3.02-2.94 (m, 1H), 2.87 (s, 3H), 2.38-2.29 (m, 1H), 2.04-1.98 (m, 1H), 1.82-1.75 (m, 2H); LCMS (ES⁺) Method 2: m/z 493 (M+H)⁺, RT 1.16 min.

### Example 135: 4-((8-((1S,6R,7R)-7-(aminomethyl)-7-(2-fluorophenyl)-3-azabicyclo[4.1.0]heptan-3-yl)imidazo[1,5-a]pyrazin-5-yl)thio)-3-chloropyridin-2-amine (Trifluoro Acetate Salt)

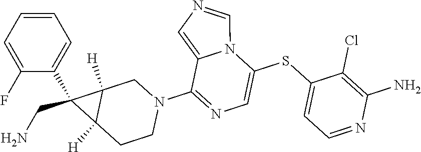 [SMILES: NC[C@]1(c2ccccc2F)[C@@H]2CCN(c3ncc(Sc4ccnc(N)c4Cl)n4cncc34)C[C@@H]21]

The above example was synthesized using the procedure reported for Example 2 step 1 using Intermediate 51 (using DIPEA in DMSO) and the same procedure reported for Example 53 step 1&2; Example 135 was obtained as a white powder (TFA salt; 5.5 mg, 80%). ¹H NMR (400 MHz, DMSO-d₆) δ 8.30 (s, 1H), 8.10 (s, 1H), 7.7 (br s, 3H), 7.63 (d, J=5.7 Hz, 1H), 7.61 (s, 1H), 7.49 (t, J=7.1 Hz, 1H), 7.41-7.36 (m, 1H), 7.25-7.19 (m, 2H), 6.76 (br s, 2H), 5.81 (d, J=5.5 Hz, 1H), 4.40-4.32 (m, 2H), 4.18 (br dd, J=14.7, 7.0 Hz, 1H), 3.24-3.18 (m, 1H), 3.12 (br d, J=4.8 Hz, 2H), 2.42-2.37 (m, 1H), 1.86-1.76 (m, 2H), 1.63-1.55 (m, 1H); LCMS (ES⁺) Method 2: m/z 496 (M+H)⁺, RT 1.10 min.

### Example 140: ((1S,6R,7S)-7-(3-fluoropyridin-2-yl)-3-(3-(pyrazolo[1,5-a]pyridin-4-yl)-1H-pyrazolo[3,4-b]pyrazin-6-yl)-3-azabicyclo[4.1.0]heptan-7-yl)methanamine (Trifluoro Acetate Salt)

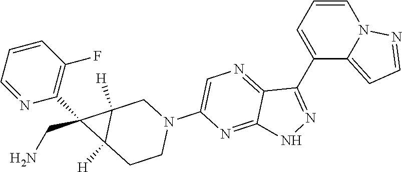 [SMILES: NC[C@]1(c2ncccc2F)[C@@H]2CCN(c3cnc4c(-c5cccn6nccc56)n[nH]c4n3)C[C@@H]21]

The above example was synthesized using the procedure reported for Example 2 using Intermediates 74, 44 and 62; Example 140 was obtained as a yellow powder (TFA salt; 6.1 mg, 56%). ¹H NMR (400 MHz, DMSO-d₆) δ 13.51 (s, 1H), 8.75 (d, J=7.0 Hz, 1H), 8.62 (d, J=7.2 Hz, 1H), 8.52 (s, 1H), 7.40 (br d, J=4.6 Hz, 1H), 8.11 (d, J=2.2 Hz, 1H), 7.74 (br t, J=9.1 Hz, 4H), 7.48-7.44 (m, 1H), 7.39 (br d, J=1.5 Hz, 1H), 7.09 (t, J=7.0 Hz, 1H), 4.48 (br d, J=13.8 Hz, 1H), 4.40 (br d, J=8.6 Hz, 1H), 3.78 (br dd, J=14.2, 6.1 Hz, 1H), 3.34-3.29 (m, 1H), 3.22-3.17 (m, 1H), 3.01-2.93 (m, 1H), 2.39-2.33 (m, 1H), 2.12-2.06 (m, 1H), 1.85-1.79 (m, 2H); LCMS (ES⁺) Method 2: m/z 456 (M+H)⁺, RT 1.15 min.

### Example 144: ((1S,6R,7S)-7-(3-fluoropyridin-2-yl)-3-(3-(8-(trifluoromethoxy)quinolin-5-yl)-1H-pyrazolo[3,4-b]pyrazin-6-yl)-3-azabicyclo[4.1.0]heptan-7-yl)methanamine (Trifluoro Acetate Salt)

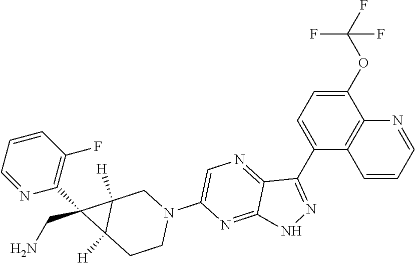 [SMILES: NC[C@]1(c2ncccc2F)[C@@H]2CCN(c3cnc4c(-c5ccc(OC(F)(F)F)c6ncccc56)n[nH]c4n3)C[C@@H]21]

The above example was synthesized using the procedure reported for Example 2 using Intermediates 74 and 44 and 5-(4,4,5,5-tetramethyl-1,3,2-dioxaborolan-2-yl)-8-(trifluoromethoxy)quinoline; Example 144 was obtained as a yellow powder (TFA salt; 7.2 mg, 48%). ¹H NMR (400 MHz, DMSO-d₆) δ 13.64 (br s, 1H), 9.34 (d, J=7.5 Hz, 1H), 9.09 (dd, J=4.1, 1.6 Hz, 1H), 8.51 (s, 1H), 8.44 (d, J=8.1 Hz, 1H), 8.40 (d, J=4.6 Hz, 1H), 7.99 (d, J=8.3 Hz, 1H), 7.77-7.72 (m, 5H), 7.48-7.44 (m, 1H), 4.49 (br d, J=14.5 Hz, 1H), 4.40 (br d, J=9.0 Hz, 1H), 3.78 (br dd, J=14.1, 6.3 Hz, 1H), 3.34-3.29 (m, 1H), 3.23-3.18 (m, 1H), 3.01-2.94 (m, 1H), 2.39-2.32 (m, 1H), 2.12-2.06 (m, 1H), 1.87-1.80 (m, 2H); LCMS (ES⁺) Method 2: m/z 551 (M+H)⁺, RT 1.24 min.

### Example 148: ((1S,6R,7S)-3-(3-(1,7-naphthyridin-5-yl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

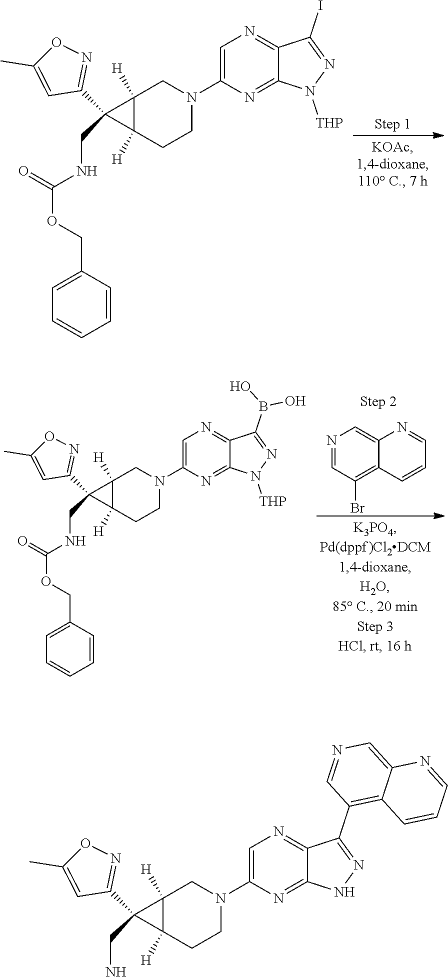

### Step 1: (6-((1S,6R,7S)-7-((((benzyloxy)carbonyl)amino)methyl)-7-(5-methylisoxazol-3-yl)-3-azabicyclo[4.1.0]heptan-3-yl)-1-(tetrahydro-2H-pyran-2-yl)-1H-pyrazolo[3,4-b]pyrazin-3-yl)boronic acid

A suspension of B2pin2 (85.3 mg, 0.34 mmol), benzyl (((1S,6R,7S)-3-(3-iodo-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate (prepared as reported in the synthesis of Example 2 step 1 using Intermediates 18 and 44; 90 mg, 0.13 mmol), KOAc (52.8 mg, 0.54 mmol) and Pd(dppf)Cl₂·DCM (22.2 mg, 0.03 mmol) in degassed 1,4-dioxane (0.5 mL) was heated at 110° C. for 7 h. After cooling the mixture was filtered on a pad of solka-floc and washed with EtOAc. The organic phase was concentrated in vacuo to afford the title compound (168 mg, 25% pure, 53%) which was used as crude in the next step. LCMS (ES⁺) Method 2: m/z 588 (M+H)⁺, RT 1.76 min.

### Step 2: benzyl (((1S,6R,7S)-3-(3-(1,7-naphthyridin-5-yl)-1-(tetrahydro-2H-pyran-2-yl)-1H-pyrazolo[3,4-b]pyrazin-6-yl)-7-(5-methylisoxazol-3-yl)-3-azabicyclo[4.1.0]heptan-7-yl)methyl)carbamate

A suspension of (6-((1S,6R,7S)-7-((((benzyloxy)carbonyl)amino)methyl)-7-(5-methylisoxazol-3-yl)-3-azabicyclo[4.1.0]heptan-3-yl)-1-(tetrahydro-2H-pyran-2-yl)-1H-pyrazolo[3,4-b]pyrazin-3-yl)boronic acid (80 mg, 0.03 mmol), 5-bromo-1,7-naphthyridine (14.2 mg, 0.07 mmol), Pd(dppf)Cl₂·DCM (5.6 mg, 0.01 mmol) and K₃PO₄ (43.4 mg, 0.20 mmol) in a mixture of degassed 1,4-dioxane (1 mL)/H₂O (0.1 mL) was heated at 85° C. for 20 min. After cooling the mixture was diluted with EtOAc (40 mL), washed with brine (2×5 mL), dried over Na₂SO₄ and concentrated in vacuo to afford a residue which was purified by flash chromatography (0-100% EtOAc+10% MeOH in petroleum ether) to obtain the title compound as a yellow solid (36 mg, 0.05 mmol, 50% pure, 79%. LCMS (ES⁺) Method 2 m/z 672 (M+H)⁺, RT 1.86 min) which was treated with conc. HCl (37%; 0.6 mL, 0.04 mmol) and stirred at rt for 16 h. After volatiles removal in vacuo the residue was purified by preparative HPLC to obtain the title compound as a yellow solid (TFA salt; 2.5 mg, 10%). ¹H NMR (400 MHz, DMSO-d₆) δ 13.71 (s, 1H), 9.48 (s, 1H), 9.45 (s, 1H), 9.37 (d, J=9.2 Hz, 1H), 9.15 (dd, J=4.0, 1.5 Hz, 1H), 8.52 (s, 1H), 7.91-7.87 (br m, 4H), 6.16 (s, 1H), 4.36 (br d, J=13.2 Hz, 2H), 3.77 (br dd, J=13.9, 6.3 Hz, 1H), 3.27-3.18 (m, 2H), 3.03-2.95 (m, 1H), 2.39 (s, 3H), 2.35-2.32 (m, 1H), 2.00-1.94 (m, 1H), 1.80-1.73 (m, 2H); LCMS (ES⁺) Method 2 m/z 454 (M+H)⁺, RT 0.92 min.

### Example 149: ((1S,6R,7S)-3-(3-(2-methoxyquinolin-6-yl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

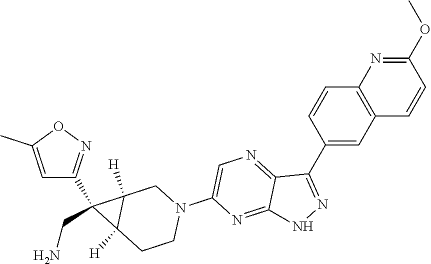 [SMILES: COc1ccc2cc(-c3n[nH]c4nc(N5CC[C@@H]6[C@H](C5)[C@@]6(CN)c5cc(C)on5)cnc34)ccc2n1]

The above example was synthesized using the procedure reported for Example 148 using 6-bromo-2-methoxyquinoline in step 2; Example 149 was obtained as a white powder (TFA salt; 1.0 mg, 5%). ¹H NMR (400 MHz, DMSO-d₆) δ 13.34 (br s, 1H), 8.86 (d, J=1.8 Hz, 1H), 8.61 (dd, J=8.8, 2.0 Hz, 1H), 8.49 (s, 1H), 8.34 (d, J=8.8 Hz, 1H), 7.90 (d, J=8.8 Hz, 1H), 7.87 (br s, 3H), 7.06 (d, J=9.0 Hz, 1H), 6.15 (s, 1H), 4.33 (br d, J=15.1 Hz, 2H), 4.01 (s, 3H), 3.76 (br dd, J=14.3, 6.6 Hz, 1H), 3.24-3.19 (m, 2H), 3.01-2.94 (m, 1H), 2.39 (s, 3H), 2.36-2.28 (m, 1H), 1.99-1.92 (m, 1H), 1.80-1.73 (m, 2H); LCMS (ES⁺) Method 2: m/z 483 (M+H)⁺, RT 1.38 min.

### Example 150: ((1S,6R,7S)-7-(5-methylisoxazol-3-yl)-3-(3-(2-(trifluoromethyl)quinolin-6-yl)-1H-pyrazolo[3,4-b]pyrazin-6-yl)-3-azabicyclo[4.1.0]heptan-7-yl)methanamine (Trifluoro Acetate Salt)

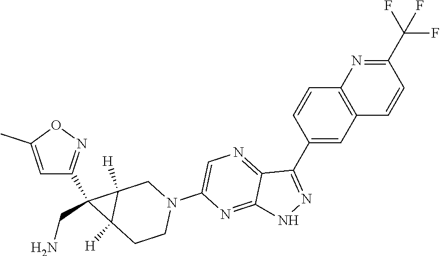 [SMILES: Cc1cc([C@@]2(CN)[C@@H]3CCN(c4cnc5c(-c6ccc7nc(C(F)(F)F)ccc7c6)n[nH]c5n4)C[C@@H]32)no1]

The above example was synthesized using the procedure reported for Example 148 using 6-bromo-2-(trifluoromethyl)quinoline in step 2; Example 150 was obtained as a white powder (TFA salt; 2.2 mg, 8%). ¹H NMR (400 MHz, DMSO-d₆) δ 13.56 (s, 1H), 9.11 (br s, 1H), 8.85 (br t, J=8.0 Hz, 2H), 8.53 (s, 1H), 8.30 (d, J=9.0 Hz, 1H), 8.01 (d, J=8.6 Hz, 1H), 7.87 (br s, 3H), 6.15 (s, 1H), 4.34 (br d, J=13.4 Hz, 2H), 3.78 (br dd, J=14.6, 6.3 Hz, 1H), 3.26-3.18 (m, 2H), 3.06-2.96 (m, 1H), 2.39 (s, 3H), 2.35-2.30 (m, 1H), 2.00-1.94 (m, 1H), 1.79-1.73 (m, 2H); LCMS (ES⁺) Method 2: m/z 521 (M+H)⁺, RT 1.51 min.

### Example 151: ((1S,6R,7S)-3-(3-(7-methoxyquinolin-3-yl)-1H-pyrazolo[3,4-b]pyrazin-6-yl)-7-(5-methylisoxazol-3-yl)-3-azabicyclo[4.1.0]heptan-7-yl)methanamine (Trifluoro Acetate Salt)

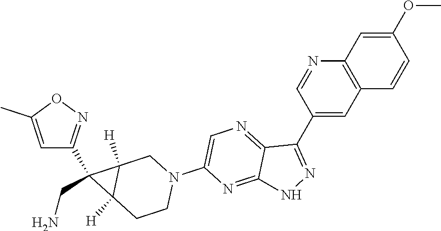

The above example was synthesized using the procedure reported for Example 148 using 3-bromo-7-methoxyquinoline in step 2; Example 151 was obtained as a yellow powder (TFA salt; 5.6 mg, 39%). ¹H NMR (400 MHz, DMSO-d₆) δ 9.92 (s, 1H), 9.79 (s, 1H), 8.53 (s, 1H), 8.43 (d, J=9.0 Hz, 1H), 7.89 (br s, 3H), 7.64-7.59 (m, 3H), 6.09 (s, 1H), 4.32 (br d, J=13.2 Hz, 2H), 4.03 (s, 3H), 3.79 (br dd, J=14.1, 6.0 Hz, 1H), 3.46-3.37 (m, 1H), 3.25-3.18 (m, 1H), 3.03-2.97 (m, 1H), 2.35 (s, 3H), 2.35-2.30 (m, 1H), 1.99-1.92 (m, 1H), 1.81-1.72 (m, 2H); LCMS (ES⁺) Method 2: m/z 483 (M+H)⁺, RT 1.07 min.

### Example 152: ((1S,6R,7S)-7-(5-methylisoxazol-3-yl)-3-(3-(7-(trifluoromethyl)quinolin-3-yl)-1H-pyrazolo[3,4-b]pyrazin-6-yl)-3-azabicyclo[4.1.0]heptan-7-yl)methanamine (Trifluoro Acetate Salt)

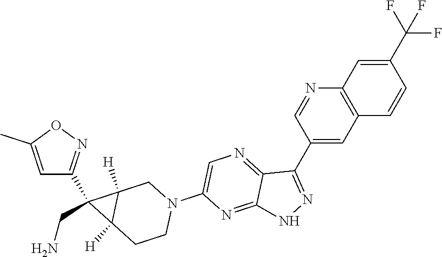 [SMILES: Cc1cc([C@@]2(CN)[C@@H]3CCN(c4cnc5c(-c6cnc7cc(C(F)(F)F)ccc7c6)n[nH]c5n4)C[C@@H]32)no1]

The above example was synthesized using the procedure reported for Example 148 using 3-bromo-7-(trifluoromethyl)quinoline in step 2; Example 152 was obtained as a yellow powder (TFA salt; 4.5 mg, 16%). ¹H NMR (400 MHz, DMSO-d₆) δ 13.66 (br s, 1H), 9.95 (d, J=2.2 Hz, 1H), 9.32 (br s, 1H), 8.55 (s, 1H), 8.42 (s, 1H), 8.39 (br d, J=8.6 Hz, 1H), 7.93 (br d, J=7.0 Hz, 1H), 7.87 (br s, 3H), 6.15 (s, 1H), 4.34 (br d, J=13.6 Hz, 2H), 3.79 (br dd, J=14.2, 6.1 Hz, 1H), 3.44-3.39 (m, 1H), 3.24-3.18 (m, 1H), 3.04-2.97 (m, 1H), 2.39 (s, 3H), 2.35-2.29 (m, 1H), 2.00-1.94 (m, 1H), 1.77-1.74 (m, 2H); LCMS (ES⁺) Method 2: m/z 521 (M+H)⁺, RT 1.53 min.

### Example 153: ((1S,6R,7R)-3-(3-(1,6-naphthyridin-5-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

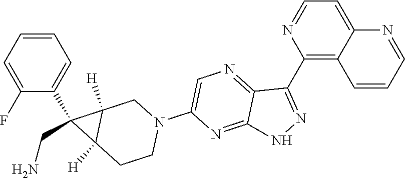 [SMILES: NC[C@]1(c2ccccc2F)[C@@H]2CCN(c3cnc4c(-c5nccc6ncccc56)n[nH]c4n3)C[C@@H]21]

The above example was synthesized using the procedure reported for Example 148 using benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported in the synthesis of Example 2 in step 1 using Intermediates 51 and 44) in step 1 and 5-bromo-1,6-naphthyridine in step 2; Example 153 was obtained as a brown powder (TFA salt; 2.0 mg, 97% pure, 14%). ¹H NMR (400 MHz, DMSO-d₆) δ 13.67 (br s, 1H), 9.17 (dd, J=4.2, 1.8 Hz, 1H), 9.01 (d, J=8.6 Hz, 1H), 8.89 (d, J=5.9 Hz, 1H), 8.49 (s, 1H), 7.99 (d, J=5.9 Hz, 1H), 7.73 (d, J=4.2 Hz, 1H), 7.70 (d, J=4.2 Hz, 1H), 7.67 (br s, 2H), 7.51-7.47 (m, 1H), 7.41-7.36 (m, 1H), 7.25-7.19 (m, 2H), 4.47 (br d, J=13.8 Hz, 1H), 4.36 (br d, J=10.7 Hz, 1H), 3.77 (br dd, J=14.1, 6.3 Hz, 1H), 3.18-3.04 (m, 2H), 3.00-2.93 (m, 1H), 2.39-2.32 (m, 1H), 1.85-1.78 (m, 2H), 1.58-1.54 (m, 1H); LCMS (ES⁺) Method 2: m/z 467 (M+H)⁺, RT 1.00 min.

### Example 154: ((1S,6R,7R)-3-(3-(2,3-dihydrofuro[2,3-b]pyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

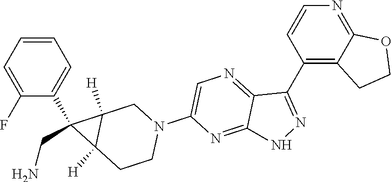 [SMILES: NC[C@]1(c2ccccc2F)[C@@H]2CCN(c3cnc4c(-c5ccnc6c5CCO6)n[nH]c4n3)C[C@@H]21]

The above example was synthesized using the procedure reported for Example 148 using benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported in the synthesis of Example 2 step 1 using Intermediates 51 and 44) in step 1 and 4-iodo-2,3-dihydrofuro[2,3-b]pyridine in step 2; HBr (33% in AcOH) was used in step 3; Example 154 was obtained as a yellow powder (TFA salt; 1.3 mg, 13%). ¹H NMR (400 MHz, DMSO-d₆) δ 13.65 (s, 1H), 8.53 (s, 1H), 8.10-8.05 (m, 2H), 7.65 (br s, 3H), 7.48 (br t, J=7.1 Hz, 1H), 7.40-7.34 (m, 1H), 7.24-7.19 (m, 2H), 4.63 (t, J=8.7 Hz, 2H), 4.45 (br d, J=14.5 Hz, 1H), 4.34 (br d, J=13.4 Hz, 1H), 3.75 (br dd, J=14.4, 6.5 Hz, 1H), 3.62-3.58 (m, 2H), 3.12-2.93 (m, 3H), 2.36-2.31 (m, 1H), 1.85-1.78 (m, 2H), 1.56-1.52 (m, 1H); LCMS (ES⁺) Method 2: m/z 458 (M+H)⁺, RT 1.16 min.

### Example 155: ((1S,6R,7R)-3-(3-(1,7-naphthyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

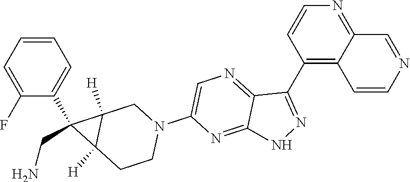 [SMILES: NC[C@]1(c2ccccc2F)[C@@H]2CCN(c3cnc4c(-c5ccnc6cnccc56)n[nH]c4n3)C[C@@H]21]

The above example was synthesized using the procedure reported for Example 148 using benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported in the synthesis of Example 2 step 1 using Intermediates 51 and 44) in step 1 and 4-bromo-1,7-naphthyridine in step 2; Example 155 was obtained as a white powder (TFA salt; 2.1 mg, 26%). ¹H NMR (400 MHz, DMSO-d₆) δ 13.89 (s, 1H), 9.48 (s, 1H), 9.22 (d, J=4.4 Hz, 1H), 9.04 (d, J=5.9 Hz, 1H), 8.75 (d, J=4.4 Hz, 1H), 8.71 (d, J=5.9 Hz, 1H), 8.58 (s, 1H), 7.68 (br s, 3H), 7.49 (br t, J=7.0 Hz, 1H), 7.41-7.35 (m, 1H), 7.25-7.20 (m, 2H), 4.48 (br d, J=14.7 Hz, 1H), 4.37 (br d, J=10.7 Hz, 1H), 3.78 (br dd, J=14.4, 6.7 Hz, 1H), 3.19-2.94 (m, 3H), 2.39-2.32 (m, 1H), 1.84-1.77 (m, 2H), 1.58-1.52 (m, 1H); LCMS (ES⁺) Method 2: m/z 467 (M+H)⁺, RT 1.00 min.

### Example 156: ((1S,6R,7S)-3-(7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methanamine (Trifluoro Acetate Salt)

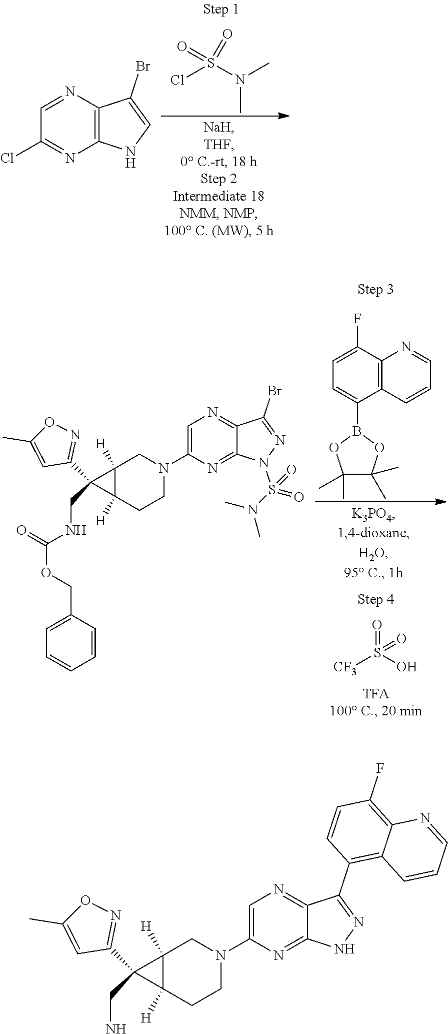

### Step 1: 7-bromo-3-chloro-N,N-dimethyl-5H-pyrrolo[2,3-b]pyrazine-5-sulfonamide

A solution of 7-bromo-3-chloro-5H-pyrrolo[2,3-b]pyrazine (200 mg, 0.86 mmol) in THF (4.3 mL) at 0° C. was treated with NaH (60% wt in mineral oil; 41.3 mg, 1.03 mmol) followed by N,N-dimethylsulfamoyl chloride (0.11 mL, 1.03 mmol) and the mixture was warmed at rt and stirred for 18 h. NH₄Cl sat. sol. was added and the mixture extracted with EtOAc (3×). The organics were concentrated in vacuo to afford a residue which was treated with DCM and then purified by flash chromatography (0-100% EtOAc in petroleum ether) to obtain the title compound as a white solid (68 mg, 23%). ¹H NMR (400 MHz, DMSO-d₆) δ 8.80 (s, 1H), 8.43 (s, 1H), 2.96 (s, 6H); LCMS (ES⁺) Method 2 m/z 338, 340 (M+H)⁺, RT 1.90 min.

### Step 2: benzyl (((1S,6R,7S)-3-(7-bromo-5-(N,N-dimethylsulfamoyl)-5H-pyrrolo[2,3-b]pyrazin-3-yl)-7-(5-methylisoxazol-3-yl)-3-azabicyclo[4.1.0]heptan-7-yl)methyl)carbamate

A solution of Intermediate 18 (25.0 mg, 0.07 mmol) in NMP (0.15 mL) was treated with 7-bromo-3-chloro-N,N-dimethyl-5H-pyrrolo[2,3-b]pyrazine-5-sulfonamide (15 mg, 0.04 mmol) and NMM (0.02 mL, 0.22 mmol) and stirred at 100° C. for 5 h under MW irradiation. After cooling the mixture was diluted with DCM and H₂O. The organic phase was concentrated in vacuo to afford a residue which was purified by flash chromatography (0-100% EtOAc in petroleum ether) to obtain the title compound (15 mg, 53%). LCMS (ES⁺) Method 2 m/z 644 (M+H)⁺, RT 2.14 min.

### Step 3: benzyl (((1S,6R,7S)-3-(5-(N,N-dimethylsulfamoyl)-7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methyl)carbamate

A suspension of 8-fluoro-5-(4,4,5,5-tetramethyl-1,3,2-dioxaborolan-2-yl)quinoline (14.0 mg, 0.05 mmol), K₃PO₄ (17.4 mg, 0.08 mmol), benzyl (((1S,6R,7S)-3-(7-bromo-5-(N,N-dimethylsulfamoyl)-5H-pyrrolo[2,3-b]pyrazin-3-yl)-7-(5-methylisoxazol-3-yl)-3-azabicyclo[4.1.0]heptan-7-yl)methyl)carbamate (14 mg, 0.02 mmol) and Pd(dppf)Cl₂·DCM (3.4 mg, 0.004 mmol) in a degassed mixture of 1,4-dioxane (0.58 mL)/H₂O (0.058 mL) was heated at 95° C. for 1 h. After cooling the mixture was diluted with EtOAc, washed with brine, dried over Na₂SO₄, and concentrated in vacuo to afford a residue which was purified by flash chromatography (0-100% EtOAc in petroleum ether) to obtain the title compound as a yellow solid (5 mg, 34%). LCMS (ES⁺) Method 2 m/z 711 (M+H)⁺, RT 2.23 min

### Step 4: ((1S,6R,7S)-3-(7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methanamine

A solution of benzyl (((1S,6R,7S)-3-(5-(N,N-dimethylsulfamoyl)-7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methyl)carbamate (5 mg, 0.01 mmol) in TFA (0.1 mL) was treated with trifluoromethanesulfonic acid (0.1 mL) and the mixture was stirred at 100° C. for 20 min. After cooling, the mixture was poured into ice and solid K₂CO₃ was slowly added until pH 10. The mixture was extracted with EtOAc, dried over Na₂SO₄, and concentrated in vacuo to afford a residue which was purified by flash chromatography (C18; 0-100% MeCN/H₂O+0.1% TFA) to obtain the title compound as a yellow solid (0.9 mg, 27%). LCMS (ES⁺) Method 2: m/z 470 (M+H)⁺, RT 1.01 min

### Example 157: ((1S,6R,7R)-3-(7-(2,4-dimethyl-2H-indazol-5-yl)-5H-pyrrolo[2,3-b]pyrazin-3-yl)-7-(2-fluorophenyl)-3-azabicyclo[4.1.0]heptan-7-yl)methanamine (Trifluoro Acetate Salt)

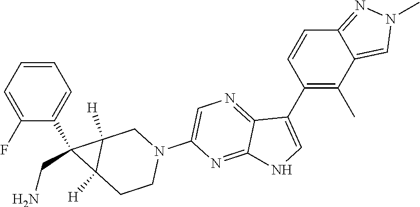 [SMILES: Cc1c(-c2c[nH]c3nc(N4CC[C@@H]5[C@H](C4)[C@@]5(CN)c4ccccc4F)cnc23)ccc2nn(C)cc12]

The above example was synthesized using the procedure reported for Example 156 using Intermediate 51 in step 2 and Intermediate 53 in step 3; Example 157 was obtained as a yellow powder (TFA salt; 1.3 mg, 22%). ¹H NMR (400 MHz, DMSO-d₆+TFA) δ 12.16 (br s, 1H), 8.52 (s, 1H), 8.20 (s, 1H), 7.71 (d, J=2.6 Hz, 1H), 7.69 (br s, 3H), 7.51-7.32 (m, 4H), 7.20-7.16 (m, 2H), 4.34 (br d, J=13.6 Hz, 1H), 4.24 (br d, J=10.3 Hz, 1H), 4.20 (s, 3H), 3.64-3.59 (m, 1H), 3.23-3.17 (m, 2H), 2.88-2.82 (m, 1H), 2.46 (s, 3H), 2.36-2.30 (m, 1H), 1.84-1.75 (m, 2H), 1.56-1.52 (m, 1H); LCMS (ES⁺) Method 2: m/z 482 (M+H)⁺, RT 1.23 min

### Example 158: ((1S,6R,7R)-3-(3-(2-chloro-3-(pyrazin-2-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

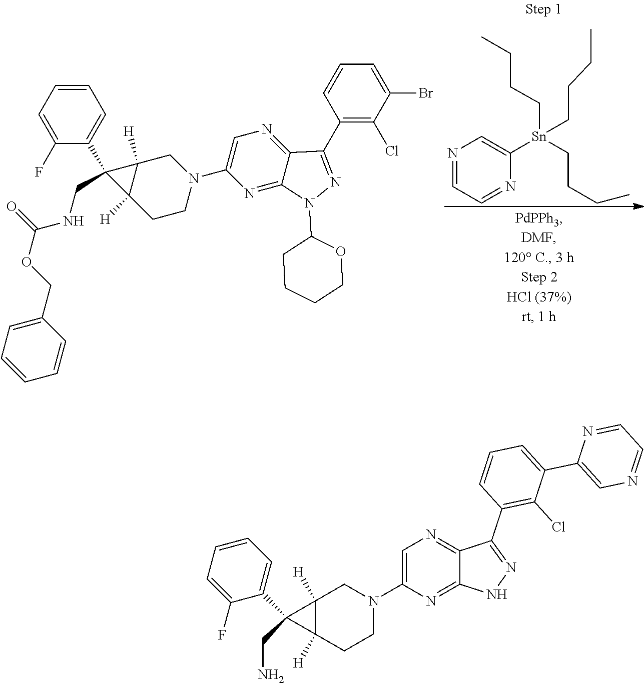 [SMILES: O=C(NC[C@]1(c2ccccc2F)[C@@H]2CCN(c3cnc4c(-c5cccc(Br)c5Cl)nn(C5CCCCO5)c4n3)C[C@@H]21)OCc1ccccc1 ; CCC[CH2][Sn]([CH2]CCC)([CH2]CCC)[c]1cnccn1 ; NC[C@]1(c2ccccc2F)[C@@H]2CCN(c3cnc4c(-c5cccc(-c6cnccn6)c5Cl)n[nH]c4n3)C[C@@H]21]

### Step 1: benzyl (((1S,6R,7R)-3-(3-(2-chloro-3-(pyrazin-2-yl)phenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate

A solution of benzyl (((1S,6R,7R)-3-(3-(3-bromo-2-chlorophenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported in the synthesis of Example 2 steps 1 & 2 using (3-bromo-2-chlorophenyl)boronic acid; 24 mg, 0.03 mmol), tributyl(pyrazin-2-yl)stannane (0.01 mL, 0.04 mmol) and Pd(PPh₃)₄(3.7 mg, 0.003 mmol) in degassed DMF (0.8 mL) was heated at 120° C. for 3 h. After cooling, the mixture was diluted with EtOAc (40 mL), washed with brine (2×5 mL), dried over Na₂SO₄, and concentrated in vacuo to afford a residue which was purified by flash chromatography (0-100% EtOAc in petroleum ether) to obtain the title compound as a yellow solid (10 mg, 50% pure, 21%). LCMS (ES⁺) Method 2: m/z 745 (M+H)⁺, RT 2.41 min.

### Step 2: ((1S,6R,7R)-3-(3-(2-chloro-3-(pyrazin-2-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

Benzyl (((1S,6R,7R)-3-(3-(2-chloro-3-(pyrazin-2-yl)phenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (10 mg, 0.01 mmol) was treated with conc. HCl (37%; 0.4 mL, 14.8 mmol) and the mixture was stirred at rt for 1 h. After volatiles removal in vacuo the residue was purified by preparative HPLC (18-100% MeCN/H₂O+0.1% TFA) to obtain the title compound as an off-white solid (TFA salt; 1.0 mg, 28%). ¹H NMR (400 MHz, DMSO-d₆) δ 13.43 (s, 1H), 8.96 (d, J=1.3 Hz, 1H), 8.82-8.81 (m, 1H), 8.72 (d, J=2.4 Hz, 1H), 8.43 (s, 1H), 7.81 (dd, J=7.4, 1.8 Hz, 1H), 7.71-7.62 (br m, 5H), 7.50-7.46 (m, 1H), 7.41-7.36 (m, 1H), 7.25-7.19 (m, 2H), 4.44 (br d, J=14.3 Hz, 1H), 4.34 (br d, J=11.2 Hz, 1H), 3.71 (br dd, J=14.1, 6.0 Hz, 1H), 3.17-3.05 (m, 2H), 2.97-2.88 (m, 1H), 2.38-2.29 (m, 1H), 1.84-1.75 (m, 2H), 1.56-1.53 (m, 1H); LCMS (ES⁺) Method 2: m/z 527 (M+H)⁺, RT 1.23 min.

### Example 163: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)pyrimidin-2-amine (Trifluoro Acetate Salt)

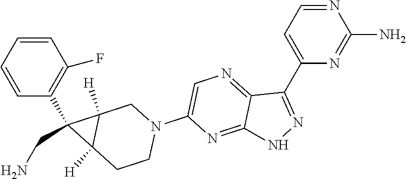 [SMILES: NC[C@]1(c2ccccc2F)[C@@H]2CCN(c3cnc4c(-c5ccnc(N)n5)n[nH]c4n3)C[C@@H]21]

The above example was synthesized using the procedure reported for Example 148 using benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported in the synthesis of Example 2 step 1 using Intermediates 51 and 44) in step 1, 4-bromo-N-(4-methoxybenzyl)pyrimidin-2-amine in step 2 and TFA; Example 163 was obtained as a yellow powder (TFA salt; 1.5 mg, 16%). ¹H NMR (400 MHz, DMSO-d₆+TFA) δ 8.59 (br s, 2H), 7.89 (d, J=4.4 Hz, 1H), 7.69 (br s, 3H), 7.52-7.47 (m, 1H), 7.40-7.35 (m, 1H), 7.23-7.17 (m, 2H), 4.41-4.30 (m, 2H), 3.87 (br dd, J=13.9, 5.7 Hz, 1H), 3.17-2.94 (m, 3H), 2.40-2.32 (m, 1H), 1.84-1.76 (m, 2H), 1.58-1.53 (m, 1H); LCMS (ES⁺) Method 2: m/z 432 (M+H)⁺, RT 0.97 min.

### Example 168: ((1S,6R,7R)-3-(3-(2-chloro-3-(oxazol-2-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

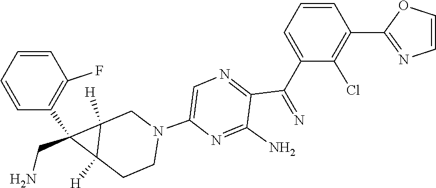

The above example was synthesized using the procedure reported for Example 158 using benzyl (((1S,6R,7R)-3-(3-(3-bromo-2-chlorophenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported in the synthesis of Example 2 steps 1 & 2) and 2-(tributylstannyl)oxazole and obtained as a yellow powder (TFA salt; 2.2 mg, 10%). ¹H NMR (400 MHz, DMSO-d₆) δ 13.45 (s, 1H), 8.42 (s, 1H), 8.35 (s, 1H), 7.99 (dd, J=7.8, 1.6 Hz, 1H), 7.81 (dd, J=7.7, 1.5 Hz, 1H), 7.68-7.61 (m, 4H), 7.51-7.46 (m, 2H), 7.40-7.36 (m, 1H), 7.24-7.19 (m, 2H), 4.44 (br d, J=13.6 Hz, 1H), 4.33 (br d, J=9.9 Hz, 1H), 3.71 (br dd, J=13.8, 6.1 Hz, 1H), 3.17-3.08 (m, 2H), 2.97-2.89 (m, 1H), 2.37-2.30 (m, 1H), 1.85-1.78 (m, 2H), 1.57-1.53 (m, 1H); LCMS (ES⁺) Method 2: m/z 516 (M+H)⁺, RT 1.39 min.

### Example 169: ((1S,6R,7R)-3-(3-(3,4-dihydro-1,5-naphthyridin-1(2H)-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

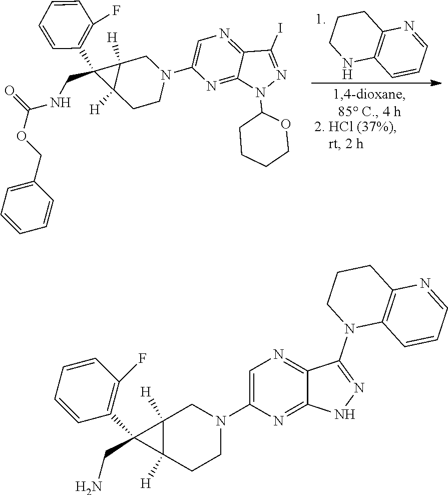 [SMILES: O=C(NC[C@]1(c2ccccc2F)[C@@H]2CCN(c3cnc4c(I)nn(C5CCCCO5)c4n3)C[C@@H]21)OCc1ccccc1 ; c1cnc2c(c1)NCCC2 ; NC[C@]1(c2ccccc2F)[C@@H]2CCN(c3cnc4c(N5CCCc6ncccc65)n[nH]c4n3)C[C@@H]21]

A solution of sodium 2-methylpropan-2-olate (12.4 mg, 0.13 mmol), [2′-(Methylamino)-2-biphenylyl]palladium(1+) methanesulfonate dicyclohexyl(2′,6′-diisopropoxy-2-biphenylyl)phosphine (1:1:1) (3.7 mg, 0.004 mmol), RuPhos (2.0 mg, 0.004 mmol), 1,2,3,4-tetrahydro-1,5-naphthyridine (6.9 mg, 0.05 mmol) and benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported for Example 2 starting from Intermediates 51 and 44; 30 mg, 0.04 mmol) in degassed 1,4-dioxane (1 mL) was heated at 85° C. for 4 h. After cooling, the mixture was filtered through a pad of cellulose and rinsed with EtOAc. The crude was treated with HCl (37%; 1 mL) for 2 h. After volatiles removal in vacuo the residue was purified by preparative HPLC to obtain the title compound as a yellow solid (TFA salt; 5.4 mg, 26%). ¹H NMR (400 MHz, DMSO-d₆) δ 12.89 (br s, 1H), 8.33 (s, 1H), 8.10 (br d, J=5.0 Hz, 1H), 7.95-7.88 (br m, 1H), 7.69-7.62 (m, 3H), 7.48 (br t, J=7.6 Hz, 1H), 7.42-7.36 (m, 2H), 7.24-7.19 (m, 2H), 4.37 (br d, J=13.8 Hz, 1H), 4.28 (br d, J=10.1 Hz, 1H), 4.03-4.00 (m, 2H), 3.73 (br dd, J=13.6, 5.9 Hz, 1H), 3.17-3.02 (m, 4H), 2.97-2.89 (m, 1H), 2.37-2.29 (m, 1H), 2.17-2.09 (m, 2H), 1.84-1.73 (m, 2H), 1.55-1.50 (m, 1H); LCMS (ES⁺) Method 2: m/z 471 (M+H)⁺, RT 1.12 min.

### Example 170: ((1S,6R,7R)-3-(3-(3-chloro-2-(oxazol-2-yl)pyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

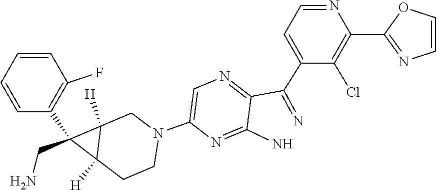 [SMILES: N=C(c1ccnc(-c2ncco2)c1Cl)c1ncc(N2CC[C@@H]3[C@H](C2)[C@@]3(CN)c2ccccc2F)nc1N]

The above example was synthesized using the procedure reported for Example 158 using benzyl (((1S,6R,7R)-3-(3-(2,3-dichloropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported in the synthesis of Example 2) and 2-(tributylstannyl)oxazole and obtained as a yellow powder (TFA salt; 2.8 mg, 89% pure, 22%). ¹H NMR (400 MHz, DMSO-d₆) δ 13.73 (s, 1H), 8.79 (d, J=4.8 Hz, 1H), 8.50 (s, 1H), 8.39 (s, 1H), 8.02 (d, J=4.8 Hz, 1H), 7.65 (br s, 3H), 7.54 (s, 1H), 7.48 (br t, J=7.8 Hz, 1H), 7.41-7.35 (m, 1H), 7.24-7.19 (m, 2H), 4.45 (br d, J=14.0 Hz, 1H), 4.34 (br d, J=10.1 Hz, 1H), 3.74 (br dd, J=14.4, 6.5 Hz, 1H), 3.16-3.04 (m, 2H), 2.99-2.92 (m, 1H), 2.38-2.30 (m, 1H), 1.84-1.76 (m, 2H), 1.59-1.52 (m, 1H); LCMS (ES⁺) Method 2: m/z 517 (M+H)⁺, RT 1.46 min.

### Example 177: ((1S,6R,7R)-7-(2,3-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

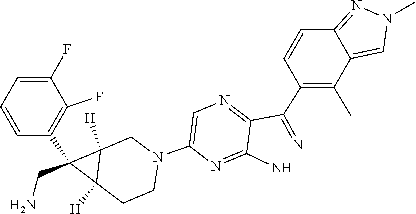 [SMILES: Cc1c(C(=N)c2ncc(N3CC[C@@H]4[C@H](C3)[C@@]4(CN)c3cccc(F)c3F)nc2N)ccc2nn(C)cc12]

The above example was synthesized using the procedure reported for Example 2 using Intermediates 88, 44, and 53; Example 177 was obtained as a yellow powder (TFA salt; 5.0 mg, 21%). ¹H NMR (400 MHz, DMSO-d₆+TFA) δ 8.50 (s, 1H), 8.41 (s, 1H), 7.74 (d, J=8.7 Hz, 1H), 7.69 (br s, 2H), 7.50 (d, J=9.0 Hz, 1H), 7.40-7.27 (m, 2H), 7.22-7.17 (m, 1H), 4.47 (br d, J=14.0 Hz, 1H), 4.34 (br d, J=10.1 Hz, 1H), 4.19 (s, 3H), 3.70 (br dd, J=14.4, 6.5 Hz, 1H), 3.19-3.08 (m, 2H), 2.96-2.89 (m, 1H), 2.64 (s, 3H), 2.35-2.30 (m, 1H), 1.87-1.78 (m, 2H), 1.62-1.58 (m, 1H); LCMS (ES⁺) Method 2: m/z 501 (M+H)⁺, RT 1.23 min.

### Example 178: 4-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-3-chloropyridin-2-amine (Trifluoro Acetate Salt)

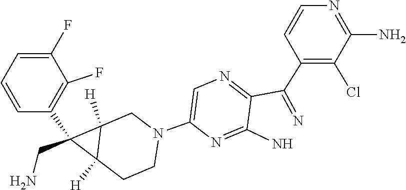 [SMILES: N=C(c1ccnc(N)c1Cl)c1ncc(N2CC[C@@H]3[C@H](C2)[C@@]3(CN)c2cccc(F)c2F)nc1N]

The above example was synthesized using the procedure reported for Example 2 using Intermediates 88, 44, and 17; Example 178 was obtained as a yellow powder (TFA salt; 3.0 mg, 38%). ¹H NMR (400 MHz, DMSO-d₆+TFA) δ 8.52 (s, 1H), 8.10 (d, J=6.6 Hz, 1H), 7.70 (br s, 3H), 7.63 (d, J=6.6 Hz, 1H), 7.40-7.34 (m, 1H), 7.31-7.27 (m, 1H), 7.22-7.17 (m, 1H), 4.42 (br d, J=14.3 Hz, 1H), 4.32 (br d, J=13.2 Hz, 1H), 3.76 (br dd, J=14.4, 6.5 Hz, 1H), 3.15-2.94 (m, 3H), 2.37-2.31 (m, 1H), 1.88-1.79 (m, 2H), 1.63-1.59 (m, 1H); LCMS (ES⁺) Method 2: m/z 483 (M+H)⁺, RT 1.05 min.

### Example 179: ((1S,6R,7S)-3-(3-(2,4-dimethyl-2H-indazol-5-yl)-1H-pyrazolo[3,4-b]pyrazin-6-yl)-7-(3-fluoropyridin-2-yl)-3-azabicyclo[4.1.0]heptan-7-yl)methanamine (Trifluoro Acetate Salt)

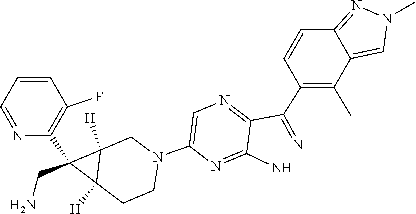 [SMILES: Cc1c(C(=N)c2ncc(N3CC[C@@H]4[C@H](C3)[C@@]4(CN)c3ncccc3F)nc2N)ccc2nn(C)cc12]

The above example was synthesized using the procedure reported for Example 2 using Intermediates 74, 44, and 53; Example 179 was obtained as a yellow powder (TFA salt; 9.5 mg, 46%). ¹H NMR (400 MHz, DMSO-d₆) δ 8.49 (s, 1H), 8.43 (s, 1H), 8.40 (br d, J=4.6 Hz, 1H), 7.77-7.72 (br m, 5H), 7.51-7.44 (m, 2H), 4.47 (br d, J=14.3 Hz, 1H), 4.38 (br d, J=8.6 Hz, 1H), 4.19 (s, 3H), 3.73 (br dd, J=14.6, 6.5 Hz, 1H), 3.35-3.28 (m, 1H), 3.26-3.19 (m, 1H), 2.97-2.90 (m, 1H), 2.64 (s, 3H), 2.38-2.31 (m, 1H), 2.11-2.05 (m, 1H), 1.83-1.79 (m, 2H); LCMS (ES⁺) Method 2: m/z 484 (M+H)⁺, RT 1.07 min.

### Example 180: ((1S,6R,7R)-7-(2-chlorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

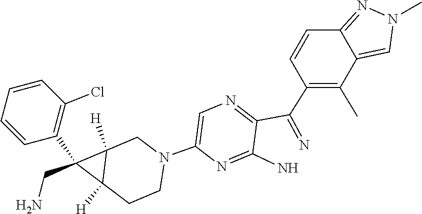 [SMILES: Cc1c(C(=N)c2ncc(N3CC[C@@H]4[C@H](C3)[C@@]4(CN)c3ccccc3Cl)nc2N)ccc2nn(C)cc12]

The above example was synthesized using the procedure reported for Example 2 using Intermediates 89, 44, and 53; Example 180 was obtained as a yellow powder (TFA salt; 4.5 mg, 20%). LCMS (ES⁺) Method 2: m/z 499 (M+H)⁺, RT 1.23 min.

### Example 181: 4-(6-((1S,6R,7R)-7-(aminomethyl)-7-(2-chlorophenyl)-3-azabicyclo[4.1.0]heptan-3-yl)-1H-pyrazolo[3,4-b]pyrazin-3-yl)-3-chloropyridin-2-amine (Trifluoro Acetate Salt)

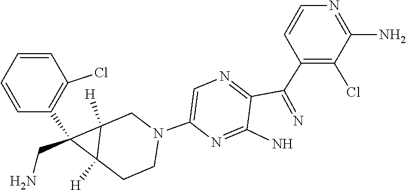 [SMILES: N=C(c1ccnc(N)c1Cl)c1ncc(N2CC[C@@H]3[C@H](C2)[C@@]3(CN)c2ccccc2Cl)nc1N]

The above example was synthesized using the procedure reported for Example 2 using Intermediates 89, 44, and 17; Example 181 was obtained as a yellow powder (TFA salt; 0.6 mg, 3%). ¹H NMR (400 MHz, DMSO-d₆+TFA) δ 8.54 (s, 1H), 8.11 (d, J=6.6 Hz, 1H), 7.70-7.63 (m, 3H), 7.58-7.54 (m, 1H), 7.51-7.48 (m, 1H), 7.39-7.34 (m, 2H), 4.45 (br d, J=13.6 Hz, 1H), 4.38-4.29 (br in, 1H), 3.79-3.74 (br in, 1H), 3.32-2.91 (br in, 3H), 2.42-2.30 (m, 1H), 1.86-1.71 (m, 2H), 1.48-1.35 (m, 1H); LCMS (ES⁺) Method 2: m/z 481 (M+H)⁺, RT 1.04 min.

### Example 182: ((1S,6R,7R)-3-(3-(2,4-dimethyl-2H-indazol-5-yl)-1H-pyrazolo[3,4-b]pyrazin-6-yl)-7-(2-(trifluoromethyl)phenyl)-3-azabicyclo[4.1.0]heptan-7-yl)methanamine (Trifluoro Acetate Salt)

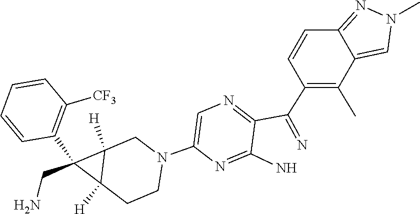 [SMILES: Cc1c(C(=N)c2ncc(N3CC[C@@H]4[C@H](C3)[C@@]4(CN)c3ccccc3C(F)(F)F)nc2N)ccc2nn(C)cc12]

The above example was synthesized using the procedure reported for Example 2 using Intermediates 90, 44, and 53; Example 182 was obtained as a yellow powder (TFA salt; 1.2 mg, 9%). ¹H NMR (400 MHz, DMSO-d₆) δ 13.25-13.19 (m, 1H), 8.50 (s, 1H), 8.44-8.39 (m, 1H), 7.79-7.67 (m, 5H), 7.64-7.55 (m, 3H), 7.51-7.49 (m, 1H), 4.58-4.52 (m, 1H), 4.36 (br d, J=13.6 Hz, 1H), 4.19 (s, 3H), 3.81-3.74 (br in, 1H), 3.19-3.13 (br in, 2H), 3.00-2.91 (m, 1H), 2.64 (s, 3H), 1.95-1.60 (m, 4H); LCMS (ES⁺) Method 2: m/z 533 (M+H)⁺, RT 1.59 min.

### Example 183: ((1S,6R,7R)-7-(2,3-difluorophenyl)-3-(3-(8-methoxyquinolin-5-yl)-1H-pyrazolo[3,4-b]pyrazin-6-yl)-3-azabicyclo[4.1.0]heptan-7-yl)methanamine (Trifluoro Acetate Salt)

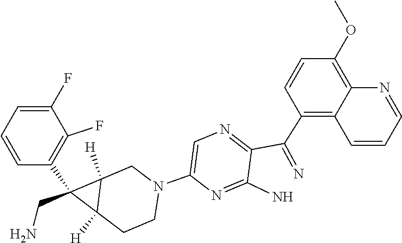 [SMILES: COc1ccc(C(=N)c2ncc(N3CC[C@@H]4[C@H](C3)[C@@]4(CN)c3cccc(F)c3F)nc2N)c2cccnc12]

The above example was synthesized using the procedure reported for Example 2 using Intermediates 88 and 44; Example 183 was obtained as a yellow powder (TFA salt; 8.6 mg, 44%). ¹H NMR (400 MHz, DMSO-d₆) δ 13.42 (br s, 1H), 9.35 (br d, J=8.1 Hz, 1H), 8.94 (dd, J=4.2, 1.5 Hz, 1H), 8.47 (s, 1H), 8.37 (d, J=8.3 Hz, 1H), 7.71-7.66 (m, 4H), 7.44-7.36 (m, 1H), 7.30-7.28 (m, 1H), 7.24-7.18 (m, 1H), 4.48 (d, J=14.3 Hz, 1H), 4.35 (br d, J=12.5 Hz, 1H), 4.06 (s, 3H), 3.74 (br dd, J=14.4, 6.6 Hz, 1H), 3.19-3.07 (m, 2H), 2.99-2.91 (m, 1H), 2.37-2.31 (m, 1H), 1.89-1.77 (m, 2H), 1.63-1.59 (m, 1H); LCMS (ES⁺) Method 2: m/z 514 (M+H)⁺, RT 0.95 min.

### Example 184: ((1S,6R,7R)-3-(3-(2,4-dimethyl-2H-indazol-5-yl)-1H-pyrazolo[3,4-b]pyrazin-6-yl)-7-(2-fluoro-3-methylphenyl)-3-azabicyclo[4.1.0]heptan-7-yl)methanamine (Trifluoro Acetate Salt)

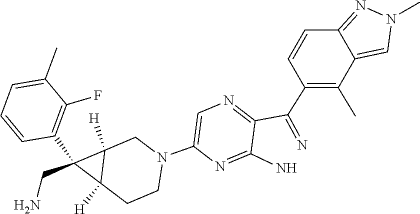 [SMILES: Cc1cccc([C@@]2(CN)[C@@H]3CCN(c4cnc(C(=N)c5ccc6nn(C)cc6c5C)c(N)n4)C[C@@H]32)c1F]

The above example was synthesized using the procedure reported for Example 2 using Intermediates 91, 44, and 53; Example 184 was obtained as a white powder (TFA salt; 5.7 mg, 41%). ¹H NMR (400 MHz, DMSO-d₆) δ 13.15 (br s, 1H), 8.49 (s, 1H), 8.41 (s, 1H), 7.73 (d, J=8.8 Hz, 1H), 7.65 (br s, 2H), 7.50 (d, J=8.8 Hz, 1H), 7.30-7.22 (m, 2H), 7.10-7.07 (m, 1H), 4.46 (br d, J=13.8 Hz, 1H), 4.33 (br d, J=9.7 Hz, 1H), 4.19 (s, 3H), 3.73-3.68 (m, 1H), 3.18-3.05 (m, 2H), 2.95-2.89 (m, 1H), 2.64 (s, 3H), 2.37-2.28 (m, 1H), 2.25 (s, 3H), 1.83-1.75 (m, 2H), 1.54-1.50 (m, 1H); LCMS (ES⁺) Method 2: m/z 497 (M+H)⁺, RT 1.27 min.

### Example 185: ((1S,6R,7S)-7-(5-cyclopropyl-3-fluoropyridin-2-yl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

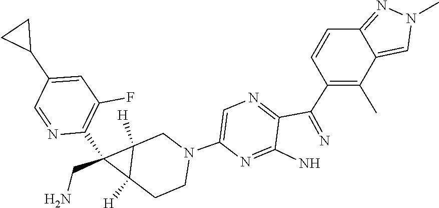 [SMILES: Cc1c(C(=N)c2ncc(N3CC[C@@H]4[C@H](C3)[C@@]4(CN)c3ncc(C4CC4)cc3F)nc2N)ccc2nn(C)cc12]

The above example was synthesized using the procedure reported for Example 2 using Intermediates 92, 44, and 53; Example 185 was obtained as a yellow powder (TFA salt; 5.6 mg, 23%). ¹H NMR (400 MHz, DMSO-d₆) δ 13.66 (br s, 1H), 8.96 (s, 1H), 8.88 (s, 1H), 8.72 (br s, 1H), 8.21-8.17 (m, 4H), 7.96 (d, J=9.0 Hz, 1H), 7.83 (d, J=11.1 Hz, 1H), 4.91 (br d, J=14.0 Hz, 1H), 4.83 (br d, J=8.8 Hz, 1H), 4.66 (s, 3H), 4.18 (br dd, J=13.7, 6.0 Hz, 1H), 3.76-3.69 (m, 1H), 3.66-3.61 (m, 1H), 3.42-3.35 (m, 1H), 3.11 (s, 3H), 2.82-2.76 (m, 1H), 2.51-2.44 (m, 2H), 2.31-2.18 (m, 2H), 1.54-1.49 (m, 2H), 1.27-1.24 (m, 2H); LCMS (ES⁺) Method 2: m/z 524 (M+H)⁺, RT 1.17 min.

### Example 199: ((1S,6R,7R)-7-(2,4-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

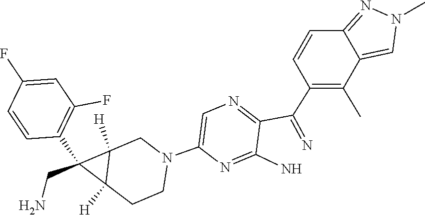 [SMILES: Cc1c(C(=N)c2ncc(N3CC[C@@H]4[C@H](C3)[C@@]4(CN)c3ccc(F)cc3F)nc2N)ccc2nn(C)cc12]

The above example was synthesized using the procedure reported for Example 2 using Intermediates 93, 44 and 53; Example 199 was obtained as a white powder (TFA salt; 6.9 mg, 95% pure, 30%). ¹H NMR (400 MHz, DMSO-d₆) δ 13.21 (s, 1H), 8.50 (s, 1H), 8.41 (s, 1H), 7.73 (br d, J=8.8 Hz, 1H), 7.64 (br s, 3H), 7.52-7.49 (m, 2H), 7.30-7.24 (m, 1H), 7.13-7.08 (m, 1H), 4.45 (br d, J=14.3 Hz, 1H), 4.33 (br d, J=8.8 Hz, 1H), 4.19 (s, 3H), 3.69 (br dd, J=14.0, 6.4 Hz, 1H), 3.14-3.03 (m, 2H), 2.95-2.89 (m, 1H), 2.64 (s, 3H), 2.36-2.29 (m, 1H), 1.81-1.75 (m, 2H), 1.55-1.52 (m, 1H); LCMS (ES⁺) Method 2: m/z 501 (M+H)⁺, RT 1.16 min.

### Example 200: ((1S,6R,7R)-7-(2,4-difluorophenyl)-3-(3-(8-methoxyquinolin-5-yl)-1H-pyrazolo[3,4-b]pyrazin-6-yl)-3-azabicyclo[4.1.0]heptan-7-yl)methanamine (Trifluoro Acetate Salt)

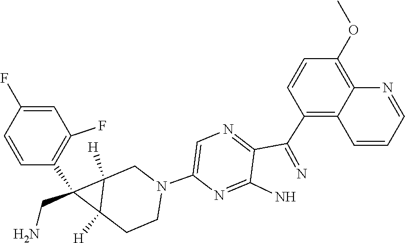 [SMILES: COc1ccc(C(=N)c2ncc(N3CC[C@@H]4[C@H](C3)[C@@]4(CN)c3ccc(F)cc3F)nc2N)c2cccnc12]

The above example was synthesized using the procedure reported for Example 2 using Intermediates 93 and 44; Example 200 was obtained as a yellow powder (TFA salt; 6.0 mg, 93% pure, 25%). ¹H NMR (400 MHz, DMSO-d₆) δ 13.38 (br s, 1H), 9.27 (d, J=7.9 Hz, 1H), 8.91 (d, J=2.6 Hz, 1H), 8.46 (s, 1H), 8.34 (d, J=8.3 Hz, 1H), 7.69-7.62 (m, 4H), 7.54-7.49 (m, 1H), 7.38 (d, J=8.3 Hz, 1H), 7.30-7.24 (m, 1H), 7.12-7.09 (m, 1H), 4.46 (br d, J=14.0 Hz, 1H), 4.35 (br d, J=10.1 Hz, 1H), 4.05 (s, 3H), 3.73 (br dd, J=14.1, 6.3 Hz, 1H), 3.16-3.03 (m, 2H), 2.98-2.91 (m, 1H), 2.37-2.31 (m, 1H), 1.84-1.75 (m, 2H), 1.55-1.52 (m, 1H); LCMS (ES⁺) Method 2: m/z 514 (M+H)⁺, RT 0.99 min.

### Example 213: ((1R,5S,6r)-3-(3-(8-fluoroquinolin-5-yl)-1H-pyrazolo[3,4-b]pyrazin-6-yl)-6-(4-methylthiazol-2-yl)-3-azabicyclo[3.1.0]hexan-6-yl)methanamine (Trifluoro Acetate Salt)

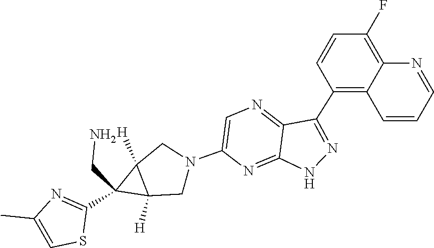 [SMILES: Cc1csc([C@@]2(CN)[C@@H]3CN(c4cnc5c(-c6ccc(F)c7ncccc67)n[nH]c5n4)C[C@@H]32)n1]

The above example was synthesized using the procedure reported for Example 91 step 3 and 4 using Intermediate 123 and 8-fluoro-5-(4,4,5,5-tetramethyl-1,3,2-dioxaborolan-2-yl)quinoline (prepared as reported in the synthesis of Intermediate 15). Example 213 was obtained as yellow powder (TFA salt, 10 mg, 66%). ¹H NMR (300 MHz, DMSO-d₆+TFA) δ 9.43-9.32 (m, 1H), 9.06 (dd, J=1.6, 4.2 Hz, 1H), 8.41 (dd, J=5.1, 8.2 Hz, 1H), 8.14 (s, 1H), 7.99 (br s, 3H), 7.87-7.70 (m, 2H), 7.25 (br d, J=1.0 Hz, 1H), 4.17-3.84 (i, 4H), 3.43-3.22 (i, 2H), 2.58-2.52 (i, 2H), 2.39 (br d, J 0.9 Hz, 3H). Method 2: m/z 473 (M+H)⁺, RT 2.03 min.

The following examples were synthesized using the above procedure with Intermediate 123 and the appropriate boronic ester or acid (commercial or, unless otherwise stated, prepared as exemplified for Intermediate 15).

| Example # | Boronic ester or acid | ¹H NMR (300 MHz) or LCMS (ES⁺) |
|---|---|---|
| 283 | Intermediate 148 | (DMSO-d₆ + TFA) δ 8.66 (d, J = 2.5 Hz, 1H), 8.20-8.07 |
| | | (m, 2H), 8.06-7.87 (m, 5H), 7.82 (d, J = 1.6 Hz, 1H), 7.24 |
| | | (br d, J = 1.0 Hz, 1H), 6.66-6.56 (m, 1H), 4.14-3.84 (m, |
| | | 4H), 3.42-3.20 (m, 2H), 2.59-2.52 (m, 2H), 2.39 (br d, |
| | | J = 0.8 Hz, 3H) |
| 441 | 5-fluoro-6-(4,4,5,5- | (DMSO-d₆ + TFA) δ 11.51 (br s, 1H), 8.30-8.19 (m, 1H), |
| | tetramethyl-1,3,2- | 8.19-8.09 (m, 2H), 7.98 (br s, 3H), 7.37-7.27 (m, 1H), |
| | dioxaborolan-2- | 7.24 (br d, J = 0.9 Hz, 1H), 6.70 (d, J = 7.3 Hz, 1H), 4.21- |
| | yl)isoquinolin-1(2H)-one | 3.78 (m, 4H), 3.48-3.15 (m, 2H), 2.58-2.53 (m, 2H), |
| | | 2.39 (br d, J = 0.7 Hz, 3H) |
| 458 | Intermediate 113 | (DMSO-d₆ + TFA) δ 8.83 (dd, J = 0.8, 7.2 Hz, 1H), 8.20- |
| | | 8.10 (m, 2H), 7.98 (br s, 3H), 7.50 (d, J = 7.2 Hz, 1H), |
| | | 7.24 (br d, J = 1.0 Hz, 1H), 6.83 (dd, J = 0.8, 2.3 Hz, 1H), |
| | | 4.12-3.89 (m, 4H), 3.32 (s, 2H), 2.58-2.52 (m, 2H), 2.39 |
| | | (br d, J = 0.8 Hz, 3H) |
| 459 | Intermediate 60 | (DMSO-d₆ + TFA) δ 9.17 (d, J = 4.8 Hz, 1H), 9.04-8.90 |
| | | (m, 1H), 8.61 (d, J = 4.7 Hz, 1H), 8.22 (s, 1H), 7.99 (br s, |
| | | 3H), 7.81-7.67 (m, 2H), 7.25 (br d, J = 0.9 Hz, 1H), 4.05 |
| | | (m, 4H), 3.45-3.21 (m, 2H), 2.58-2.53 (m, 2H), 2.39 (br |
| | | d, J = 0.7 Hz, 3H) |
| 460 | Intermediate 82 | (DMSO-d₆ + TFA) δ 9.12 (dd, J = 1.8, 11.7 Hz, 2H), 8.22 |
| | | (dd, J = 8.7, 11.8 Hz, 2H), 8.13 (s, 1H), 7.98 (br s, 3H), |
| | | 7.24 (br d, J = 1.0 Hz, 1H), 4.12-3.90 (m, 4H), 3.43-3.18 |
| | | (m, 2H), 2.56-2.52 (m, 2H), 2.39 (br d, J = 0.9 Hz, 3H) |
| 461 | Intermediate 54 | (DMSO-d₆ + TFA) δ 8.10 (s, 1H), 8.05-7.90 (m, 5H), |
| | | 7.25 (br d, J = 0.9 Hz, 1H), 4.16-3.82 (m, 4H), 3.49-3.16 |
| | | (m, 2H), 2.88 (s, 3H), 2.56-2.53 (m, 2H), 2.39 (br d, |
| | | J = 0.8 Hz, 3H) |
| 462 | 6-(4,4,5,5-tetramethyl- | (DMSO-d₆) δ 11.26 (br s, 1H), 8.62 (br d, J = 1.2 Hz, |
| | 1,3,2-dioxaborolan-2-yl)-2- | 1H), 8.44 (dd, J = 1.5, 8.4 Hz, 1H), 8.29 (d, J = 8.4 Hz, |
| | (2- | 1H), 8.20 (s, 1H), 7.98 (br s, 3H), 7.25 (br d, J = 0.9 Hz, |
| | trimethylsilylethoxymethyl) | 1H), 7.23-7.16 (m, 1H), 6.63 (d, J = 7.2 Hz, 1H), 4.16- |
| | isoquinolin-1-one (final | 3.83 (m, 4H), 3.40-3.22 (m, 2H), 2.56-2.52 (m, 2H), |
| | deprotection carried out | 2.39 (br d, J = 0.8 Hz, 3H) |
| | using TFA in DCM) | |
| 463 | Intermediate 136 | (DMSO-d₆) δ 9.92 (br d, J = 0.9 Hz, 1H), 8.11 (s, 1H), |
| | | 8.05-7.85 (m, 5H), 7.25 (br d, J = 1.0 Hz, 1H), 4.11-3.87 |
| | | (m, 4H), 3.38-3.24 (m, 2H), 2.57-2.52 (m, 2H), 2.39 (br |
| | | d, J = 0.9 Hz, 3H) |
| 464 | Intermediate 71 | (DMSO-d₆ + TFA) δ 9.07 (br s, 1H), 8.40-7.84 (m, 6H), |
| | | 7.55 (d, J = 8.7 Hz, 1H), 7.23 (br s, 1H), 3.92-4.09 (m, |
| | | 4H), 3.25-3.40 (m, 2H), 2.58-2.52 (m, 2H), 2.38 (s, 3H) |
| 465 | Intermediate 138 | (DMSO-d₆ + TFA) δ: 9.19 (s, 1H), 8.10-8.20 (m, 3H), |
| | | 7.94-8.03 (m, 3H), 7.70 (dd, J = 9.1, 0.6 Hz, 1H), 7.23 (d, |
| | | J = 1.0 Hz, 1H), 3.93-4.08 (m, 4H), 3.27-3.37 (m, 2H), |
| | | 2.58-2.52 (m, 2H), 2.38 (s, 3H) |
| 466 | Intermediate 137c | (DMSO-d₆ + TFA) δ 9.10 (s, 1H), 8.43-7.89 (m, 5H), |
| | | 7.84 (s, 2H), 7.24 (br d, J = 0.9 Hz, 1H), 4.14-3.85 (m, |
| | | 4H), 3.41-3.21 (m, 2H), 2.58-2.52 (m, 2H), 2.39 (br d, |
| | | J = 0.8 Hz, 3H) |
| 467 | Intermediate 139 | (DMSO-d₆ + TFA) δ 8.20 (dd, J = 9.1, 12.7 Hz, 2H), 8.15 |
| | | (s, 1H), 7.98 (br s, 3H), 7.25 (br d, J = 1.0 Hz, 1H), 4.21- |
| | | 3.80 (m, 4H), 3.43-3.19 (m, 2H), 2.56-2.52 (m, 2H), |
| | | 2.39 (br d, J = 0.9 Hz, 3H) |
| 476 | Intermediate 141 | (DMSO-d₆ + TFA) δ 8.23 (d, J = 5.1 Hz, 1H), 8.12 (s, 1H), |
| | | 7.97 (br s, 3H), 7.49 (d, J = 5.1 Hz, 1H), 7.24 (br d, J = 1.0 |
| | | Hz, 1H), 4.16-3.83 (m, 7H), 3.43-3.19 (m, 2H), 2.58- |
| | | 2.52 (m, 2H), 2.38 (br d, J = 0.9 Hz, 3H) |
| 477 | 3-fluoro-2-methyl-4- | (DMSO-d₆) δ 13.73 (br s, 1H), 8.42 (d, J = 5.0 Hz, 1H), |
| | (4,4,5,5-tetramethyl-1,3,2- | 8.17 (s, 1H), 8.10 (t, J = 5.3 Hz, 1H), 7.93-8.01 (m, 3H), |
| | dioxaborolan-2-yl)pyridine | 7.26 (s, 1H), 3.78-4.10 (m, 4H), 3.31 (br d, J = 5.5 Hz, |
| | | 2H), 2.55 (br d, J = 3.3 Hz, 3H), 2.39 (s, 3H) |
| 478 | Intermediate 142 | (DMSO-d₆ + TFA) δ 8.72 (s, 1H), 8.09 (s, 1H), 7.98 (br |
| | | s, 3H), 7.83-7.68 (m, 2H), 7.24 (br d, J = 0.9 Hz, 1H), |
| | | 5.91 (s, 2H), 4.12-3.88 (m, 4H), 3.40-3.19 (m, 2H), 2.55- |
| | | 2.52 (m, 2H), 2.39 (s, 3H) |
| 479 | Intermediate 143 | (DMSO-d₆ + TFA) δ 8.85 (d, J = 8.6 Hz, 1H), 8.37 (d, |
| | | J = 8.7 Hz, 1H), 8.16 (s, 1H), 7.98 (br s, 3H), 7.25 (br d, |
| | | J = 1.0 Hz, 1H), 4.17-3.83 (m, 4H), 3.43-3.17 (m, 2H), |
| | | 2.57-2.52 (m, 2H), 2.39 (br d, J = 0.9 Hz, 3H) |
| 480 | Intermediate 144 | (DMSO-d₆ + TFA) δ 8.18 (s, 1H), 8.08 (s, 1H), 7.97 (br |
| | | s, 3H), 7.83-7.71 (m, 2H), 7.24 (br d, J = 0.9 Hz, 1H), |
| | | 4.13 (s, 3H), 4.09-3.87 (m, 4H), 3.41-3.20 (m, 2H), 2.58- |
| | | 2.52 (m, 2H), 2.39 (br d, J = 0.8 Hz, 3H) |
| 482 | Intermediate 62 | (DMSO-d₆ + TFA) δ 8.67 (dd, J = 17.7, 7.0 Hz, 2H), 8.14 |
| | | (s, 1H), 8.07 (d, J = 2.2 Hz, 1H), 7.98 (br s, 3H), 7.40 (d, |
| | | J = 1.7 Hz, 1H), 7.19 (s, 1H), 7.06 (t, J = 7.1 Hz, 1H), 3.91- |
| | | 4.11 (m, 4H), 3.28-3.39 (m, 2H), 2.57-2.52 (m, 2H), |
| | | 2.37 (s, 3H) |
| 483 | 7-chloro-1-methyl-6- | (DMSO-d₆ + TFA) δ 9.64 (s, 1H), 8.07 (s, 1H), 7.85-8.03 |
| | (4,4,5,5-tetramethyl-1,3,2- | (m, 5H), 7.21 (s, 1H), 4.33 (s, 3H), 3.90-4.09 (m, 4H), |
| | dioxaborolan-2-yl)-1H- | 3.27-3.38 (m, 2H), 2.57-2.52 (m, 2H), 2.38 (s, 3H) |
| | benzo[d]imidazole | |
| 503 | 1,3-dimethyl-5-(4,4,5,5- | (DMSO-d₆ + TFA) δ 8.20 (dd, J = 1.5, 8.2 Hz, 1H), 8.14 |
| | tetramethyl-1,3,2- | (s, 1H), 8.02 (br d, J = 1.2 Hz, 1H), 7.98 (br s, 3H), 7.32- |
| | dioxaborolan-2- | 7.18 (m, 2H), 4.16-3.82 (m, 4H), 3.40 (s, 3H), 3.38 (s, |
| | yl)benzimidazol-2-one | 3H), 3.35-3.25 (m, 2H), 2.57-2.52 (m, 2H), 2.39 (br d, |
| | | J = 0.9 Hz, 3H) |
| 504 | Intermediate 161 | (DMSO-d₆ + TFA) δ 9.92 (br d, J = 8.6 Hz, 1H), 9.54 (br |
| | | s, 1H), 9.32 (br dd, J = 1.5, 4.8 Hz, 1H), 8.76 (dd, J = 7.9, |
| | | 25.7 Hz, 2H), 8.29 (br s, 1H), 8.21 (s, 1H), 8.13-7.85 (m, |
| | | 4H), 7.25 (br d, J = 1.0 Hz, 1H), 4.16-3.89 (m, 4H), 3.41- |
| | | 3.24 (m, 2H), 2.59-2.53 (m, 2H), 2.39 (br d, J = 0.8 Hz, |
| | | 3H) |
| 505 | Intermediate 147 | (DMSO-d₆ + TFA) δ 8.21 (br d, J = 0.8 Hz, 1H), 8.07 (s, |
| | | 1H), 7.97 (br s, 3H), 7.73 (d, J = 8.5 Hz, 1H), 7.65 (dd, |
| | | J = 0.8, 8.5 Hz, 1H), 7.24 (br d, J = 1.0 Hz, 1H), 4.14-3.84 |
| | | (m, 4H), 3.43-3.18 (m, 2H), 2.54-2.52 (m, 2H), 2.39 (br |
| | | d, J = 0.7 Hz, 3H) |
| 506 | Intermediate 137d | (DMSO-d₆ + TFA) δ 8.57 (s, 1H), 8.51-8.06 (m, 2H), |
| | | 8.06-7.89 (m, 5H), 7.24 (br d, J = 1.0 Hz, 1H), 4.14-3.83 |
| | | (m, 4H), 3.43-3.19 (m, 2H), 2.58-2.52 (m, 2H), 2.39 (br |
| | | d, J = 0.8 Hz, 3H) |
| 512 | 3-chloro-4-(4,4,5,5- | (DMSO-d₆ + TFA) δ 8.18 (br s, 1H), 8.13-8.06 (m, 2H), |
| | tetramethyl-1,3,2- | 8.04-7.85 (m, 5H), 7.58 (br s, 1H), 7.24 (br d, J = 1.0 Hz, |
| | dioxaborolan-2- | 1H), 4.13-3.85 (m, 4H), 3.40-3.20 (m, 2H), 2.56-2.52 |
| | yl)benzamide | (m, 2H), 2.38 (br d, J = 0.8 Hz, 3H) |
| 513 | 1-methyl-5-(4,4,5,5- | (DMSO-d₆ + TFA) δ 8.33 (br dd, J = 1.5, 8.2 Hz, 1H), 8.25 |
| | tetramethyl-1,3,2- | (s, 1H), 8.11 (s, 1H), 7.98 (br s, 3H), 7.24 (br d, J = 0.9 |
| | dioxaborolan-2-yl)indolin- | Hz, 1H), 7.10 (d, J = 8.2 Hz, 1H), 4.11-3.83 (m, 4H), 3.66 |
| | 2-one | (s, 2H), 3.39-3.25 (m, 2H), 3.17 (s, 3H), 2.57-2.52 (m, |
| | | 2H), 2.39 (br d, J = 0.8 Hz, 3H) |
| 517 | (2-amino-3-chloropyridin- | (DMSO-d₆) δ 13.58 (s, 1H), 8.18-7.92 (m, 5H), 7.34- |
| | 4-yl)boronic acid | 7.22 (m, 1H), 7.02 (d, J = 5.1 Hz, 1H), 6.53 (br s, 2H), |
| | | 4.11-3.96 (m, 4H), 3.38-3.26 (m, 2H), 2.55-2.52 (m, |
| | | 2H), 2.40 (s, 3H) |
| 518 | 4-chloro-5-(4,4,5,5- | (DMSO-d₆ + TFA) δ 8.35 (br d, J = 0.6 Hz, 1H), 8.08 (s, |
| | tetramethyl-1,3,2- | 1H), 7.97 (br s, 3H), 7.94-7.83 (m, 2H), 7.22 (br d, J = 0.9 |
| | dioxaborolan-2-yl)-2-(2,2,2- | Hz, 1H), 5.55 (q, J = 9.0 Hz, 2H), 4.11-3.88 (m, 4H), |
| | trifluoroethyl)-2H-indazole | 3.40-3.21 (m, 2H), 2.61-2.52 (m, 2H), 2.38 (br d, J = 0.8 |
| | (prepared as reported for | Hz, 3H) |
| | Intermediate 45 using | |
| | Cs₂CO₃ and 1,1,1-trifluoro- | |
| | 2-iodo-ethane in step 2) | |
| 519 | 3-chloro-4-(4,4,5,5- | (DMSO-d₆ + TFA) δ 8.88 (s, 1H), 8.72 (d, J = 5.2 Hz, 1H), |
| | tetramethyl-1,3,2- | 8.20 (d, J = 5.2 Hz, 1H), 8.16 (s, 1H), 7.98 (br s, 3H), 7.24 |
| | dioxaborolan-2-yl)pyridine | (br d, J = 1.0 Hz, 1H), 4.17-3.79 (m, 4H), 3.42-3.19 (m, |
| | | 2H), 2.59-2.52 (m, 2H), 2.39 (br d, J = 0.8 Hz, 3H) |
| 528 | Intermediate 180a | Method 2A: m/z 518, 520 (M + H)⁺, RT 2.37 min |

### Example 221: ((1R,5S,6s)-3-(3-(4-chloro-2-methyl-2H-indazol-5-yl)-1H-pyrazolo[3,4-b]pyrazin-6-yl)-6-(2,6-difluorophenyl)-3-azabicyclo[3.1.0]hexan-6-yl)methanamine (Trifluoro Acetate Salt)

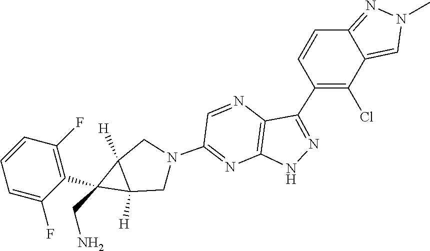 [SMILES: Cn1cc2c(Cl)c(-c3n[nH]c4nc(N5C[C@@H]6[C@H](C5)[C@@]6(CN)c5c(F)cccc5F)cnc34)ccc2n1]

The above example was synthesized using the procedure reported for Example 2 using Intermediates 44, 45 and 154; Example 221 was obtained as a white powder (TFA salt; 5.1 mg, 33%). ¹H NMR (400 MHz, DMSO-d₆) δ 13.39 (br s, 1H), 8.56 (s, 1H), 8.11 (s, 1H), 7.80 (br s, 3H), 7.69-7.62 (m, 2H), 7.52-7.45 (m, 1H), 7.17 (br t, J=8.4 Hz, 2H), 4.24 (s, 3H), 4.07 (br d, J=12.3 Hz, 2H), 3.93 (br d, J=12.1 Hz, 2H), 3.03-2.96 (m, 2H), 2.38-2.32 (m, 2H); LCMS (ES⁺) Method 2: m/z 507 (M+H)⁺, RT 1.10 min.

### Example 256: ((1S,6R,7R)-3-(3-(2-chloro-4-(1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

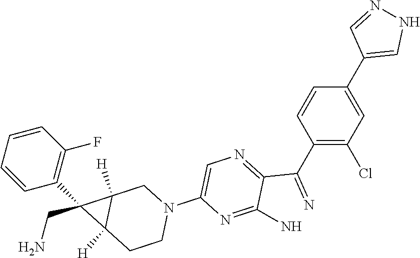 [SMILES: N=C(c1ccc(-c2cn[nH]c2)cc1Cl)c1ncc(N2CC[C@@H]3[C@H](C2)[C@@]3(CN)c2ccccc2F)nc1N]

The above example was synthesized as reported for Example 2 using Intermediates 51, 44 and (4-bromo-2-chlorophenyl)boronic acid, followed by reaction with 4-(4,4,5,5-tetramethyl-1,3,2-dioxaborolan-2-yl)-1-trityl-1H-pyrazole in the same conditions of step 1 (Pd(dppf)Cl₂·DCM as catalyst) of Example 111; Example 256 was obtained as a white powder (TFA salt; 2.5 mg, 22%). ¹H NMR (400 MHz, DMSO-d₆) δ 13.32 (br s, 1H), 8.43 (s, 1H), 8.22 (br s, 2H), 7.87 (s, 1H), 7.77-7.70 (m, 2H), 7.66 (br s, 3H), 7.50-7.47 (m, 1H), 7.40-7.35 (m, 1H), 7.24-7.19 (m, 2H), 4.46 (br d, J=14.3 Hz, 1H), 4.34 (br d, J=10.1 Hz, 1H), 3.74-3.70 (m, 1H), 3.19-3.04 (m, 2H), 2.98-2.89 (m, 1H), 2.38-2.31 (m, 1H), 1.84-1.77 (m, 2H), 1.56-1.52 (m, 1H); LCMS (ES⁺) Method 2: m/z 515 (M+H)⁺, RT 1.15 min.

### Example 263: ((1S,6R,7R)-3-(3-(3-chloro-2-(oxazol-5-yl)pyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

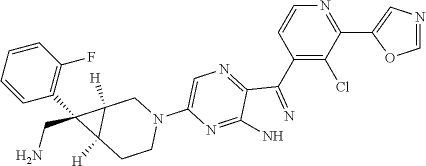 [SMILES: N=C(c1ccnc(-c2cnco2)c1Cl)c1ncc(N2CC[C@@H]3[C@H](C2)[C@@]3(CN)c2ccccc2F)nc1N]

The above example was synthesized as reported for Example 2 using Intermediates 51, 44 and (2,3-dichloropyridin-4-yl)boronic acid, followed by reaction with 5-(4,4,5,5-tetramethyl-1,3,2-dioxaborolan-2-yl)oxazole using with the conditions used in step 1 of Example 111; Example 263 was obtained as a brown powder (TFA salt; 0.4 mg, 95% pure, 1.4%). ¹H NMR (400 MHz, DMSO-d₆) δ 13.70 (s, 1H), 8.77 (d, J=4.8 Hz, 1H), 8.67 (s, 1H), 8.49 (s, 1H), 8.07 (s, 1H), 7.85 (d, J=4.8 Hz, 1H), 7.65 (br s, 3H), 7.48 (br t, J=7.5 Hz, 1H), 7.41-7.36 (m, 1H), 7.24-7.19 (m, 2H), 4.44 (br d, J=14.3 Hz, 1H), 4.34 (br d, J=10.5 Hz, 1H), 3.74 (br dd, J=14.3, 6.4 Hz, 1H), 3.19-3.04 (m, 2H), 2.99-2.93 (m, 1H), 2.40-2.33 (m, 1H), 1.83-1.77 (m, 2H), 1.57-1.53 (m, 1H); LCMS (ES⁺) Method 2: m/z 517 (M+H)⁺, RT 1.06 min.

### Example 264: ((1S,6R,7R)-3-(3-(2-chloro-4-(oxazol-2-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

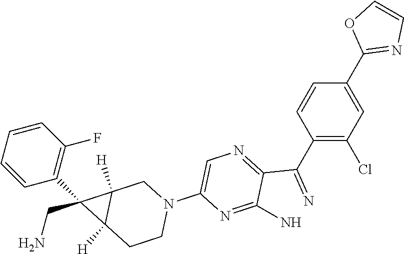 [SMILES: N=C(c1ccc(-c2ncco2)cc1Cl)c1ncc(N2CC[C@@H]3[C@H](C2)[C@@]3(CN)c2ccccc2F)nc1N]

The above example was synthesized as reported for Example 2 using Intermediates 51, 44 and (4-bromo-2-chlorophenyl)boronic acid followed by reaction of 2-(tributylstannyl)oxazole using the conditions of Example 111, step 1, with Pd(PPh₃)₂Cl₂ in THF instead of Pd(PPh₃)₄ in DMF; Example 264 was obtained as a white powder (TFA salt; 3.0 mg, 43%). ¹H NMR (400 MHz, DMSO-d₆) δ 13.51 (br s, 1H), 8.46 (s, 1H), 8.33 (s, 1H), 8.14 (d, J=1.5 Hz, 1H), 8.08 (d, J=8.1 Hz, 1H), 8.02 (d, J=8.1 Hz, 1H), 7.66 (br s, 3H), 7.50-7.46 (m, 2H), 7.41-7.35 (m, 1H), 7.24-7.19 (m, 2H), 4.45 (br d, J=14.9 Hz, 1H), 4.34 (br d, J=10.3 Hz, 1H), 3.73 (br dd, J=14.2, 6.2 Hz, 1H), 3.18-3.03 (m, 2H), 2.98-2.91 (m, 1H), 2.38-2.30 (m, 1H), 1.84-1.77 (m, 2H), 1.57-1.53 (m, 1H); LCMS (ES⁺) Method 2: m/z 516 (M+H)⁺, RT 1.30 min.

### Example 265: ((1S,6R,7R)-3-(3-(3-chloro-2-(1-methyl-1H-pyrazol-3-yl)pyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

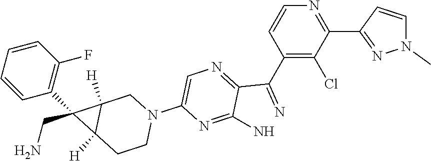 [SMILES: Cn1ccc(-c2nccc(C(=N)c3ncc(N4CC[C@@H]5[C@H](C4)[C@@]5(CN)c4ccccc4F)nc3N)c2Cl)n1]

The above example was synthesized as reported for Example 2 using Intermediates 51, 44 and (2,3-dichloropyridin-4-yl)boronic acid) followed by the reaction with 1-methyl-3-(tributylstannyl)-1H-pyrazole using the conditions of step 1 of Example 111; Example 265 was obtained as a yellow powder (TFA salt; 1.6 mg, 98% pure, 7%). ¹H NMR (400 MHz, DMSO-d₆) δ 13.61 (s, 1H), 8.66 (d, J=4.8 Hz, 1H), 8.47 (s, 1H), 7.81 (d, J=2.2 Hz, 1H), 7.71-7.70 (m, 1H), 7.66 (br s, 3H), 7.49 (br t, J=7.0 Hz, 1H), 7.41-7.35 (m, 1H), 7.24-7.19 (m, 2H), 6.76 (d, J=2.2 Hz, 1H), 4.45 (br d, J=13.8 Hz, 1H), 4.34 (br d, J=9.9 Hz, 1H), 3.94 (s, 3H), 3.73 (br dd, J=14.5, 6.6 Hz, 1H), 3.19-3.04 (m, 2H), 2.99-2.91 (m, 1H), 2.39-2.30 (m, 1H), 1.83-1.78 (m, 2H), 1.57-1.53 (m, 1H); LCMS (ES⁺) Method 2: m/z 530 (M+H)⁺, RT 1.07 min.

### Example 266: 5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)quinoline-8-carboxylic acid (Trifluoro Acetate Salt)

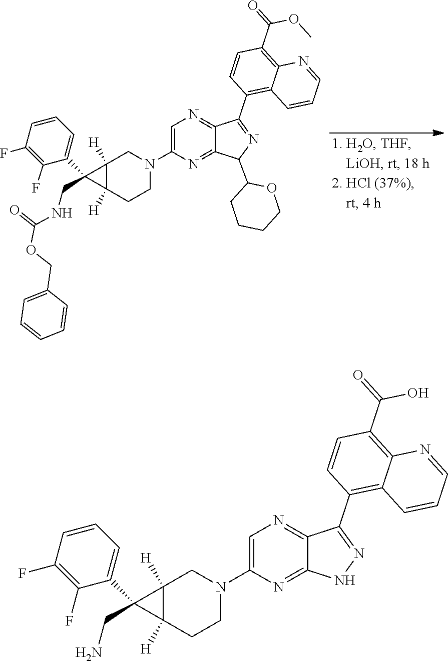 [SMILES: COC(=O)c1ccc(C2=NC(C3CCCCO3)c3nc(N4CC[C@@H]5[C@H](C4)[C@@]5(CNC(=O)OCc4ccccc4)c4cccc(F)c4F)cnc32)c2cccnc12 ; NC[C@]1(c2cccc(F)c2F)[C@@H]2CCN(c3cnc4c(-c5ccc(C(=O)O)c6ncccc56)n[nH]c4n3)C[C@@H]21]

### Step 1: 5-(6-((1S,6R,7R)-7-((((benzyloxy)carbonyl)amino)methyl)-7-(2,3-difluorophenyl)-3-azabicyclo[4.1.0]heptan-3-yl)-1-(tetrahydro-2H-pyran-2-yl)-1H-pyrazolo[3,4-b]pyrazin-3-yl)quinoline-8-carboxylic acid

A solution of methyl 5-(6-((1S,6R,7R)-7-((((benzyloxy)carbonyl)amino)methyl)-7-(2,3-difluorophenyl)-3-azabicyclo[4.1.0]heptan-3-yl)-1-(tetrahydro-2H-pyran-2-yl)-1H-pyrazolo[3,4-b]pyrazin-3-yl)quinoline-8-carboxylate (prepared as reported for Example 2 using Intermediates 44, 88 and methyl 5-(4,4,5,5-tetramethyl-1,3,2-dioxaborolan-2-yl)quinoline-8-carboxylate; 46 mg, 0.06 mmol) in THF (0.5 mL) was treated with a solution of LiOH (2.9 mg, 0.12 mmol) in H₂O (0.1 mL) and stirred at rt for 18 h. The reaction mixture was concentrated under reduced pressure, diluted with EtOAc and acidified with 1N HCl aq. sol. The organic phase was dried over Na₂SO₄, filtered, and concentrated in vacuo to afford the title compound as a yellow solid (45 mg, 99%). LCMS (ES⁺) Method 2: m/z 746 (M+H)⁺, RT 2.43 min.

### Step 2: 5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)quinoline-8-carboxylic acid

5-(6-((1S,6R,7R)-7-((((benzyloxy)carbonyl)amino)methyl)-7-(2,3-difluorophenyl)-3-azabicyclo[4.1.0]heptan-3-yl)-1-(tetrahydro-2H-pyran-2-yl)-1H-pyrazolo[3,4-b]pyrazin-3-yl)quinoline-8-carboxylic acid (20.0 mg, 0.03 mmol) was treated with conc. HCl (37%; 0.5 mL, 0.03 mmol) and the mixture was stirred at rt for 4 h. After volatiles removal in vacuo the residue was purified by preparative HPLC to obtain the title compound as a yellow solid (TFA salt; 7.9 mg, 46%). ¹H NMR (400 MHz, DMSO-d₆) δ 13.80 (s, 1H), 9.69 (br d, J=8.3 Hz, 1H), 9.19 (d, J=4.2 Hz, 1H), 8.76 (d, J=7.7 Hz, 1H), 8.70 (d, J=7.5 Hz, 1H), 8.56 (s, 1H), 7.92 (dd, J=8.8, 4.4 Hz, 1H), 7.67 (br s, 3H), 7.43-7.36 (m, 1H), 7.29 (d, J=6.4 Hz, 1H), 7.22-7.19 (m, 1H), 4.49 (br d, J=13.8 Hz, 1H), 4.37 (br d, J=13.4 Hz, 1H), 3.77 (br dd, J=14.5, 6.4 Hz, 1H), 3.21-3.04 (m, 2H), 3.01-2.95 (m, 1H), 2.38-2.33 (m, 1H), 1.89-1.80 (m, 2H), 1.64-1.60 (m, 1H); LCMS (ES⁺) Method 2: m/z 528 (M+H)⁺, RT 1.14 min.

### Example 267: 5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-N-methylquinoline-8-carboxamide (Trifluoro Acetate Salt)

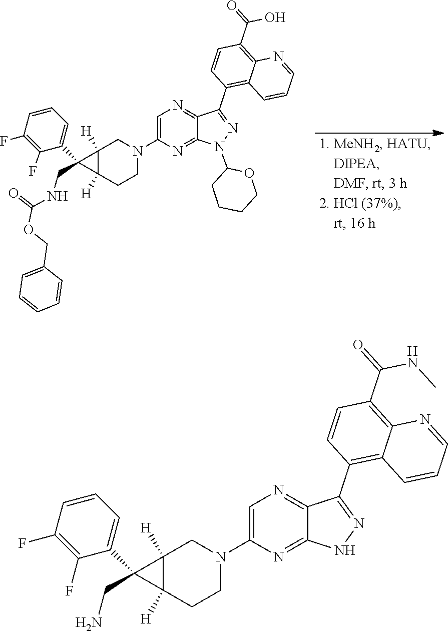 [SMILES: O=C(NC[C@]1(c2cccc(F)c2F)[C@@H]2CCN(c3cnc4c(-c5ccc(C(=O)O)c6ncccc56)nn(C5CCCCO5)c4n3)C[C@@H]21)OCc1ccccc1 ; CNC(=O)c1ccc(-c2n[nH]c3nc(N4CC[C@@H]5[C@H](C4)[C@@]5(CN)c4cccc(F)c4F)cnc23)c2cccnc12]

### Step 1: benzyl (((1S,6R,7R)-7-(2,3-difluorophenyl)-3-(3-(8-(methylcarbamoyl)quinolin-5-yl)-1-(tetrahydro-2H-pyran-2-yl)-1H-pyrazolo[3,4-b]pyrazin-6-yl)-3-azabicyclo[4.1.0]heptan-7-yl)methyl)carbamate

A solution of 5-(6-((1S,6R,7R)-7-((((benzyloxy)carbonyl)amino)methyl)-7-(2,3-difluorophenyl)-3-azabicyclo[4.1.0]heptan-3-yl)-1-(tetrahydro-2H-pyran-2-yl)-1H-pyrazolo[3,4-b]pyrazin-3-yl)quinoline-8-carboxylic acid (prepared as reported for Example 266 in step 1; 30.0 mg, 0.04 mmol), HATU (18.4 mg, 0.05 mmol), methanamine hydrochloride (5.4 mg, 0.08 mmol) and DIPEA (0.03 mL, 0.16 mmol) in DMF (0.2 mL) was stirred at rt for 3 h. The reaction mixture was diluted with EtOAc, washed with 10% citric acid, NaHCO₃ sat sol, brine, dried over Na₂SO₄, filtered, and concentrated in vacuo to afford a residue which was purified by flash chromatography (0-100% EtOAc in petroleum ether) to obtain the title compound as a yellow solid (15 mg, 49%). LCMS (ES⁺) Method 2: m/z 759 (M+H)⁺, RT 2.43 min.

### Step 2: 5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-N-methylquinoline-8-carboxamide

Benzyl (((1S,6R,7R)-7-(2,3-difluorophenyl)-3-(3-(8-(methylcarbamoyl)quinolin-5-yl)-1-(tetrahydro-2H-pyran-2-yl)-1H-pyrazolo[3,4-b]pyrazin-6-yl)-3-azabicyclo[4.1.0]heptan-7-yl)methyl)carbamate (15.0 mg, 0.02 mmol) was treated with conc. HCl (37%; 0.4 mL, 0.03 mmol) and the mixture was stirred at rt for 16 h. After volatiles removal in vacuo the residue was purified by preparative HPLC to obtain the title compound as a yellow solid (TFA salt; 6.8 mg, 52%). ¹H NMR (400 MHz, DMSO-d₆) δ 13.67 (s, 1H), 10.71 (br d, J=4.8 Hz, 1H), 9.42 (d, J=8.3 Hz, 1H), 9.10 (d, J=4.0 Hz, 1H), 8.67 (d, J=7.7 Hz, 1H), 8.53 (s, 1H), 8.47 (d, J=7.7 Hz, 1H), 7.73 (br dd, J=8.8, 4.2 Hz, 1H), 7.67 (br s, 3H), 7.41-7.32 (m, 1H), 7.29 (d, J=6.5 Hz, 1H), 7.28-7.21 (m, 1H), 4.50 (br d, J=14.3 Hz, 1H), 4.37 (br d, J=9.0 Hz, 1H), 3.75 (br dd, J=14.1, 6.3 Hz, 1H), 3.20-3.06 (m, 2H), 3.02-2.94 (m, 4H), 2.37-2.32 (m, 1H), 1.88-1.76 (m, 2H), 1.63-1.59 (m, 1H); LCMS (ES⁺) Method 2: m/z 541 (M+H)⁺, RT 1.18 min.

### Example 268: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2(1H)-one (Trifluoro Acetate Salt)

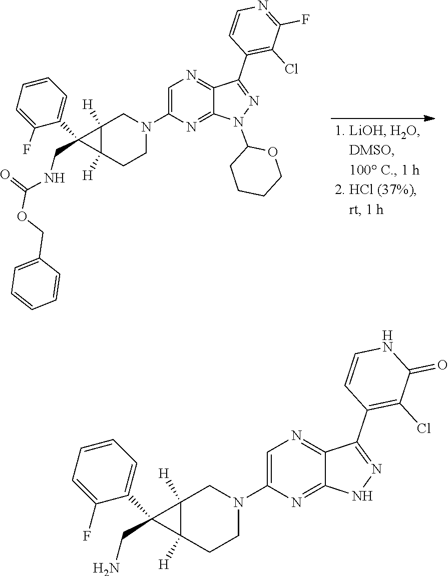 [SMILES: O=C(NC[C@]1(c2ccccc2F)[C@@H]2CCN(c3cnc4c(-c5ccnc(F)c5Cl)nn(C5CCCCO5)c4n3)C[C@@H]21)OCc1ccccc1 ; NC[C@]1(c2ccccc2F)[C@@H]2CCN(c3cnc4c(-c5cc[nH]c(=O)c5Cl)n[nH]c4n3)C[C@@H]21]

A solution of benzyl (((1S,6R,7R)-3-(3-(3-chloro-2-fluoropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported for Example 2 using Intermediates 44, 51 and (3-chloro-2-fluoropyridin-4-yl)boronic acid) in a mixture of DMSO (0.17 mL) and H₂O (0.04 mL) was treated with LiOH (5.2 mg, 0.22 mmol) and stirred at 100° C. for 1 h. After cooling, the reaction mixture was treated with conc. HCl (37%; 0.3 mL, 0.02 mmol) and the mixture was stirred at rt for 1 h. After volatiles removal in vacuo the residue was purified by preparative HPLC to obtain the title compound as a white solid (TFA salt; 0.8 mg, 8%). ¹H NMR (400 MHz, DMSO-d₆) δ 13.62 (s, 1H), 12.24 (s, 1H), 8.47 (s, 1H), 7.64 (br s, 3H), 7.51-7.48 (m, 2H), 7.41-7.35 (m, 1H), 7.24-7.19 (m, 2H), 6.69 (br d, J=7.0 Hz, 1H), 4.44 (br d, J=14.0 Hz, 1H), 4.33 (br d, J=10.1 Hz, 1H), 3.76-3.69 (m, 1H), 3.17-3.03 (m, 2H), 2.97-2.91 (m, 1H), 2.37-2.30 (m, 1H), 1.83-1.78 (m, 2H), 1.55-1.52 (m, 1H); LCMS (ES⁺) Method 2: m/z 466 (M+H)⁺, RT 0.92 min.

### Example 269: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one (Trifluoro Acetate Salt)

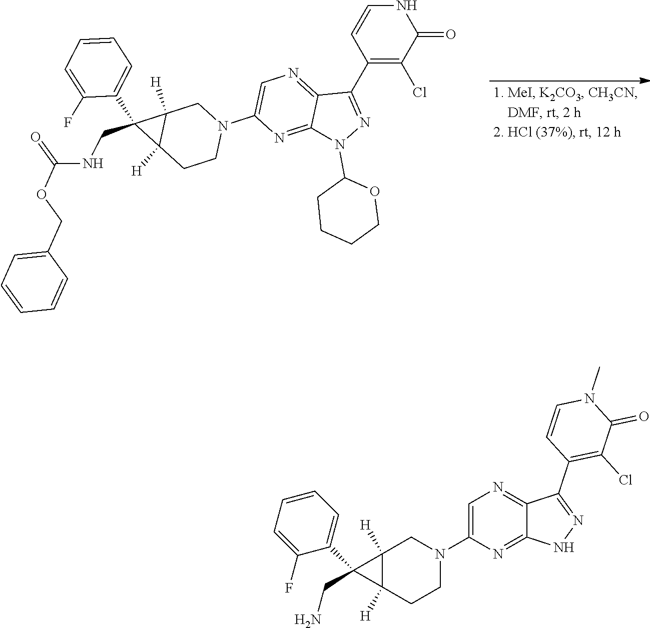 [SMILES: O=C(NC[C@]1(c2ccccc2F)[C@@H]2CCN(c3cnc4c(-c5cc[nH]c(=O)c5Cl)nn(C5CCCCO5)c4n3)C[C@@H]21)OCc1ccccc1 ; Cn1ccc(-c2n[nH]c3nc(N4CC[C@@H]5[C@H](C4)[C@@]5(CN)c4ccccc4F)cnc23)c(Cl)c1=O]

### Step 1: benzyl (((1S,6R,7R)-3-(3-(3-chloro-1-methyl-2-oxo-1,2-dihydropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate

A solution of benzyl (((1S,6R,7R)-3-(3-(3-chloro-2-oxo-1,2-dihydropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported for Example 268 in step 1; 13.0 mg, 0.02 mmol) in a mixture of MeCN (0.19 mL) and DMF (0.19 mL) was treated with K₂CO₃ (5.8 mg, 0.04 mmol) and Mel (4.0 mL, 0.03 mmol) and stirred at rt for 2 h. The reaction mixture was dilute in H₂O and DCM. The organic phase was concentrated in vacuo to afford the title compound as a yellow solid (8 mg, 60%). LCMS (ES⁺) Method 2: m/z 698 (M+H)⁺, RT 2.18 min.

### Step 2: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one

A solution of benzyl (((1S,6R,7R)-3-(3-(3-chloro-1-methyl-2-oxo-1,2-dihydropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (8 mg, 0.01 mmol) was treated with conc. HCl (37%; 0.3 mL, 0.01 mmol) and the mixture was stirred at rt for 12 h. After volatiles removal in vacuo the residue was purified by preparative HPLC to obtain the title compound as a white solid (TFA salt; 1.3 mg, 24%). ¹H NMR (400 MHz, DMSO-d₆) δ 13.63 (s, 1H), 8.47 (s, 1H), 7.82 (d, J=7.0 Hz, 1H), 7.64 (br s, 3H), 7.48 (br t, J=7.5 Hz, 1H), 7.41-7.35 (m, 1H), 7.24-7.19 (m, 2H), 6.74 (d, J=6.8 Hz, 1H), 4.45 (br d, J=14.7 Hz, 1H), 4.33 (br d, J=9.7 Hz, 1H), 3.72 (br dd, J=14.4, 6.5 Hz, 1H), 3.57 (s, 3H), 3.18-3.02 (m, 2H), 2.97-2.88 (m, 1H), 2.38-2.30 (m, 1H), 1.84-1.77 (m, 2H), 1.55-1.52 (m, 1H); LCMS (ES⁺) Method 2: m/z 480 (M+H)⁺, RT 0.94 min.

### Example 270: ((1S,6R,7R)-7-(2-fluorophenyl)-3-(3-(spiro[cyclobutane-1,3′-pyrrolo[3,2-b]pyridin]-1′(2′H)-yl)-1H-pyrazolo[3,4-b]pyrazin-6-yl)-3-azabicyclo[4.1.0]heptan-7-yl)methanamine (Trifluoro Acetate Salt)

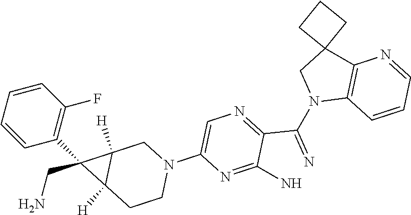 [SMILES: N=C(c1ncc(N2CC[C@@H]3[C@H](C2)[C@@]3(CN)c2ccccc2F)nc1N)N1CC2(CCC2)c2ncccc21]

The above example was synthesized using the procedure reported for Example 169 using 1′,2′-dihydrospiro[cyclobutane-1,3′-pyrrolo[3,2-b]pyridine]; Example 270 was obtained as a yellow powder (TFA salt; 10.0 mg, 46%). ¹H NMR (400 MHz, DMSO-d₆) δ 12.27 (s, 1H), 8.31 (s, 1H), 8.11 (d, J=8.1 Hz, 1H), 8.02 (d, J=4.8 Hz, 1H), 7.65 (br s, 3H), 7.48 (br t, J=7.5 Hz, 1H), 7.41-7.35 (m, 1H), 7.24-7.17 (m, 3H), 4.60 (s, 2H), 4.41 (br d, J=14.3 Hz, 1H), 4.31 (br d, J=10.7 Hz, 1H), 3.75-3.70 (m, 1H), 3.18-2.99 (m, 2H), 2.95-2.90 (m, 1H), 2.82-2.75 (m, 2H), 2.38-2.25 (m, 3H), 2.19-2.00 (m, 2H), 1.83-1.75 (m, 2H), 1.54-1.50 (m, 1H); LCMS (ES⁺) Method 2: m/z 497 (M+H)⁺, RT 1.13 min.

### Example 271: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzoic acid (Trifluoro Acetate Salt)

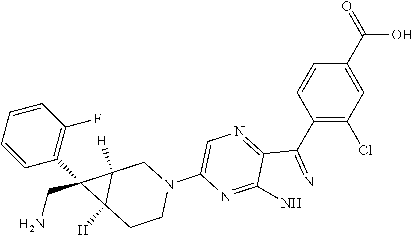 [SMILES: N=C(c1ccc(C(=O)O)cc1Cl)c1ncc(N2CC[C@@H]3[C@H](C2)[C@@]3(CN)c2ccccc2F)nc1N]

The above example was synthesized using the procedure reported for Example 266 using Intermediates 44, 51 and (2-chloro-4-(methoxycarbonyl)phenyl)boronic acid (prepared as reported for the synthesis of Intermediate 15); Example 271 was obtained as a yellow powder (TFA salt; 5.8 mg, 98% pure, 41%). ¹H NMR (400 MHz, DMSO-d₆) δ 13.53 (s, 1H), 13.44 (br s, 1H), 8.46 (s, 1H), 8.07 (s, 1H), 8.03-7.97 (m, 2H), 7.65 (br s, 3H), 7.48 (br t, J=7.8 Hz, 1H), 7.41-7.35 (m, 1H), 7.24-7.19 (m, 2H), 4.45 (br d, J=14.0 Hz, 1H), 4.33 (br d, J=8.6 Hz, 1H), 3.72 (br dd, J=14.6, 6.5 Hz, 1H), 3.18-3.02 (m, 2H), 2.97-2.90 (m, 1H), 2.37-2.31 (m, 1H), 1.83-1.75 (m, 2H), 1.56-1.52 (m, 1H); LCMS (ES⁺) Method 2: m/z 493 (M+H)⁺, RT 1.12 min.

### Example 273: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-methylbenzamide (Trifluoro Acetate Salt)

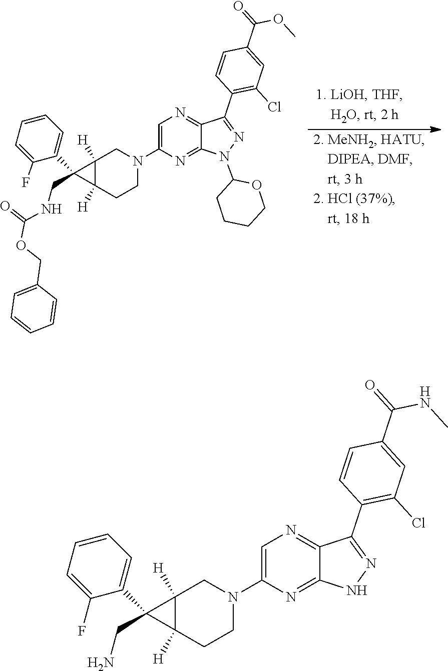 [SMILES: COC(=O)c1ccc(-c2nn(C3CCCCO3)c3nc(N4CC[C@@H]5[C@H](C4)[C@@]5(CNC(=O)OCc4ccccc4)c4ccccc4F)cnc23)c(Cl)c1 ; CNC(=O)c1ccc(-c2n[nH]c3nc(N4CC[C@@H]5[C@H](C4)[C@@]5(CN)c4ccccc4F)cnc23)c(Cl)c1]

The title compound was prepared following procedure reported for Example 2 using methyl 4-(6-((1S,6R,7R)-7-((((benzyloxy)carbonyl)amino)methyl)-7-(2-fluorophenyl)-3-azabicyclo[4.1.0]heptan-3-yl)-1-(tetrahydro-2H-pyran-2-yl)-1H-pyrazolo[3,4-b]pyrazin-3-yl)-3-chlorobenzoate (prepared as reported for Example 2 using Intermediates 44, 51 and methyl 3-chloro-4-(4,4,5,5-tetramethyl-1,3,2-dioxaborolan-2-yl)benzoate). After hydrolysis using the conditions reported for Example 266, a brown oil (78 mg, 88%) was obtained (LCMS (ES⁺) Method 2: m/z 711 (M+H)⁺, RT 2.38 min). The procedure reported for Example 267 in step 1 afforded a crude residue (LCMS (ES⁺) Method 2: m/z 724 (M+H)⁺, RT 2.31 min) which was subjected to step 2 of the same Example and finally obtained as a yellow solid (TFA salt; 1.7 mg, 16%). ¹H NMR (400 MHz, DMSO-d₆) δ 13.48 (br s, 1H), 8.66 (br d, J=4.8 Hz, 1H), 8.45 (s, 1H), 8.04 (s, 1H), 7.91 (s, 2H), 7.66 (br s, 3H), 7.48 (br t, J=7.7 Hz, 1H), 7.40-7.35 (m, 1H), 7.24-7.19 (m, 2H), 4.44 (br d, J=14.5 Hz, 1H), 4.33 (br d, J=9.7 Hz, 1H), 3.75-3.70 (m, 1H), 3.19-3.03 (m, 2H), 2.97-2.90 (m, 1H), 2.82 (d, J=4.4 Hz, 3H), 2.37-2.31 (m, 1H), 1.83-1.76 (m, 2H), 1.56-1.52 (m, 1H); LCMS (ES⁺) Method 2: m/z 506 (M+H)⁺, RT 1.08 min.

### Example 275: ((1R,5S,6r)-3-(3-(4-chloro-2-methyl-2H-indazol-5-yl)-1H-pyrazolo[3,4-b]pyrazin-6-yl)-6-(5-methylisoxazol-3-yl)-3-azabicyclo[3.1.0]hexan-6-yl)methanamine (Trifluoro Acetate Salt)

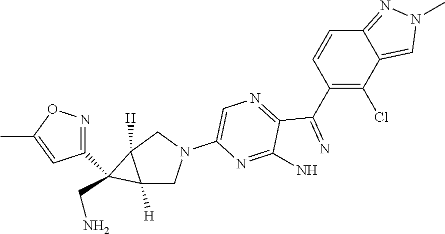

The above example was synthesized using the procedure reported for Example 2 using Intermediates 44, 45, and 94; Example 275 was obtained as a yellow powder (TFA salt; 8.5 mg, 56%). ¹H NMR (400 MHz, DMSO-d₆) δ 13.39 (br s, 1H), 8.56 (s, 1H), 8.07 (s, 1H), 7.92 (br s, 3H), 7.68 (d, J=9.0 Hz, 1H), 7.62 (d, J=9.0 Hz, 1H), 6.24 (s, 1H), 4.23 (s, 3H), 4.00-3.90 (br in, 4H), 3.26-3.17 (m, 2H), 2.44-2.42 (br in, 5H); LCMS (ES⁺) Method 2: m/z 476 (M+H)⁺, RT 0.94 min.

### Example 276: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzamide (Trifluoro Acetate Salt)

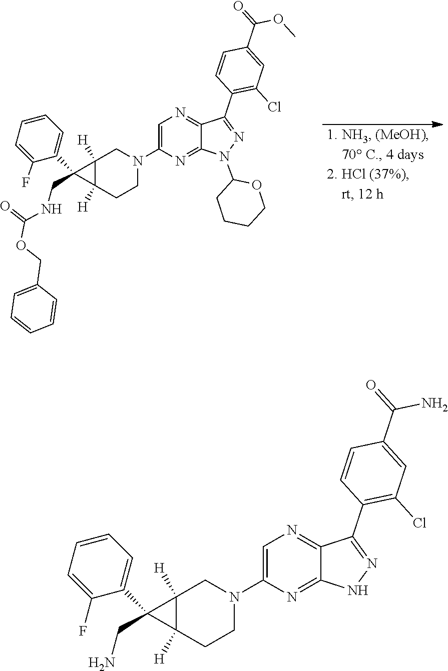 [SMILES: COC(=O)c1ccc(-c2nn(C3CCCCO3)c3nc(N4CC[C@@H]5[C@H](C4)[C@@]5(CNC(=O)OCc4ccccc4)c4ccccc4F)cnc23)c(Cl)c1 ; NC[C@]1(c2ccccc2F)[C@@H]2CCN(c3cnc4c(-c5ccc(C(N)=O)cc5Cl)n[nH]c4n3)C[C@@H]21]

A solution of Example 273 starting material (37.0 mg, 0.05 mmol) in ammonia (7N in MeOH; 0.8 mL, 5.6 mmol) was stirred at 70° C. for 4 days. After cooling to rt the solvent was removed under reduced pressure to afford a residue which was purified by flash chromatography (0-100% EtOAc in petroleum ether) to obtain the title compound (10 mg, 28%). LCMS (ES⁺) Method 2: m/z 710 (M+H)⁺, RT 2.27 min. After deprotection, following the procedure reported for step 2 of Example 267, a yellow solid (TFA salt; 0.3 mg, 4%) was obtained. ¹H NMR (400 MHz, DMSO-d₆) δ 13.48 (br s, 1H), 8.45 (s, 1H), 8.17 (br s, 1H), 8.09 (d, J=1.3 Hz, 1H), 7.95 (d, J=8.2 Hz, 1H), 7.90 (d, J=8.3 Hz, 1H), 7.65 (br s, 3H), 7.60 (br s, 1H), 7.48 (br t, J=7.0 Hz, 1H), 7.41-7.35 (m, 1H), 7.24-7.19 (m, 2H), 4.45 (br d, J=14.3 Hz, 1H), 4.33 (br d, J=10.5 Hz, 1H), 3.72 (br dd, J=14.4, 6.5 Hz, 1H), 3.19-3.02 (m, 2H), 2.97-2.89 (m, 1H), 2.38-2.31 (m, 1H), 1.84-1.77 (m, 2H), 1.56-1.52 (m, 1H); LCMS (ES⁺) Method 2: m/z 492 (M+H)⁺, RT 1.03 min.

### Example 277: 3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzoic acid (Trifluoro Acetate Salt)

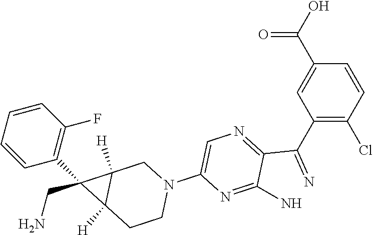 [SMILES: N=C(c1cc(C(=O)O)ccc1Cl)c1ncc(N2CC[C@@H]3[C@H](C2)[C@@]3(CN)c2ccccc2F)nc1N]

The above example was synthesized using the procedure reported for Example 266 using Intermediates 44, 51 and (2-chloro-5-(methoxycarbonyl)phenyl)boronic acid; Example 277 was obtained as a yellow powder (TFA salt; 3.0 mg, 10%). ¹H NMR (400 MHz, DMSO-d₆) δ 13.54 (s, 1H), 13.35 (br s, 1H), 8.47 (s, 1H), 8.40 (d, J=2.0 Hz, 1H), 7.98 (dd, J=8.3, 2.2 Hz, 1H), 7.75 (d, J=8.3 Hz, 1H), 7.65 (br s, 3H), 7.48 (br t, J=7.7 Hz, 1H), 7.41-7.35 (m, 1H), 7.24-7.19 (m, 2H), 4.46 (br d, J=13.8 Hz, 1H), 4.35 (br d, J=9.6 Hz, 1H), 3.75-3.69 (m, 1H), 3.19-3.03 (m, 2H), 2.97-2.91 (m, 1H), 2.38-2.31 (m, 1H), 1.84-1.77 (m, 2H), 1.58-1.52 (m, 1H); LCMS (ES⁺) Method 2: m/z 493 (M+H)⁺, RT 1.18 min.

### Example 279: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-cyclopropylquinoline-8-carboxamide (Trifluoro Acetate Salt)

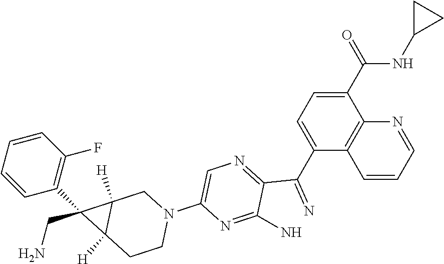 [SMILES: N=C(c1ncc(N2CC[C@@H]3[C@H](C2)[C@@]3(CN)c2ccccc2F)nc1N)c1ccc(C(=O)NC2CC2)c2ncccc12]

The above example was synthesized using the procedure reported for Example 273 using Intermediates 44, 51 and methyl 5-(4,4,5,5-tetramethyl-1,3,2-dioxaborolan-2-yl)quinoline-8-carboxylate, using cyclopropanamine for step 2; Example 279 was obtained as a yellow powder (TFA salt; 5.8 mg, 19%). ¹H NMR (400 MHz, DMSO-d₆) δ 13.66 (br s, 1H), 10.84 (d, J=4.4 Hz, 1H), 9.42 (br d, J=8.8 Hz, 1H), 9.08 (dd, J=4.2, 1.8 Hz, 1H), 8.64 (d, J=7.9 Hz, 1H), 8.52 (s, 1H), 8.48 (d, J=7.7 Hz, 1H), 7.72 (br dd, J=8.8, 4.2 Hz, 1H), 7.66 (br s, 3H), 7.49 (br t, J=6.9 Hz, 1H), 7.41-7.36 (m, 1H), 7.25-7.19 (m, 2H), 4.48 (br d, J=14.3 Hz, 1H), 4.36 (br d, J=10.5 Hz, 1H), 3.77 (br dd, J=14.4, 6.3 Hz, 1H), 3.17-2.94 (m, 4H), 2.38-2.35 (m, 1H), 1.85-1.77 (m, 2H), 1.57-1.53 (m, 1H), 0.85-0.81 (m, 2H), 0.68-0.64 (m, 2H); LCMS (ES⁺) Method 2: m/z 549 (M+H)⁺, RT 1.34 min.

### Example 281: 3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzamide (Trifluoro Acetate Salt)

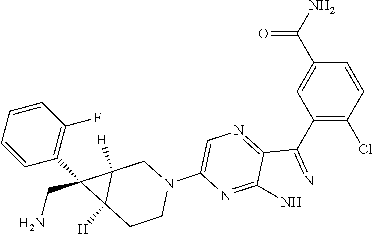 [SMILES: N=C(c1cc(C(N)=O)ccc1Cl)c1ncc(N2CC[C@@H]3[C@H](C2)[C@@]3(CN)c2ccccc2F)nc1N]

The above example was synthesized using the procedure reported for Example 276 using Intermediates 44, 51 and methyl 4-chloro-3-(4,4,5,5-tetramethyl-1,3,2-dioxaborolan-2-yl)benzoate (prepared as reported for the synthesis of Intermediate 15) as starting material; Example 281 was obtained as a yellow powder (TFA salt; 6.0 mg, 51%). ¹H NMR (400 MHz, DMSO-d₆) δ 13.45 (br s, 1H), 8.44 (s, 1H), 8.25 (s, 1H), 8.12 (br s, 1H), 7.95 (br d, J=7.9 Hz, 1H), 7.70 (br d, J=8.1 Hz, 1H), 7.65 (br s, 3H), 7.51-7.46 (m, 2H), 7.41-7.35 (m, 1H), 7.24-7.19 (m, 2H), 4.45 (br d, J=14.0 Hz, 1H), 4.34 (br d, J=9.2 Hz, 1H), 3.75-3.70 (m, 1H), 3.20-3.02 (m, 2H), 2.98-2.90 (m, 1H), 2.38-2.31 (m, 1H), 1.84-1.75 (m, 2H), 1.56-1.52 (m, 1H); LCMS (ES⁺) Method 2: m/z 492 (M+H)⁺, RT 1.10 min.

### Example 282: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-(methylsulfonyl)quinoline-8-carboxamide (Trifluoro Acetate Salt)

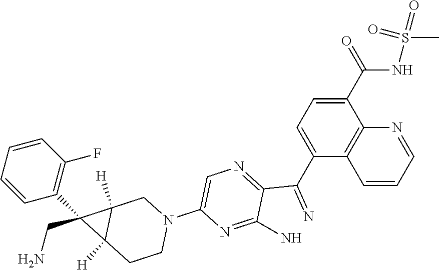 [SMILES: CS(=O)(=O)NC(=O)c1ccc(C(=N)c2ncc(N3CC[C@@H]4[C@H](C3)[C@@]4(CN)c3ccccc3F)nc2N)c2cccnc12]

The above example was synthesized using the procedure reported for Example 267 using Intermediates 44, 51 and methyl 5-(4,4,5,5-tetramethyl-1,3,2-dioxaborolan-2-yl)quinoline-8-carboxylate; methanesulfonamide, EDC-HCl and DMAP in DMF were used for the formation of the acylsulfonamide (in step 1, Example 267); Example 282 was obtained as a yellow powder (TFA salt; 3.4 mg, 10%). ¹H NMR (400 MHz, DMSO-d₆) δ 14.64 (s, 1H), 13.72 (s, 1H), 9.53 (d, J=8.6 Hz, 1H), 9.11 (dd, J=4.2, 1.5 Hz, 1H), 8.72 (d, J=7.9 Hz, 1H), 8.57 (d, J=7.9 Hz, 1H), 8.48 (s, 1H), 7.79 (dd, J=8.8, 4.2 Hz, 1H), 7.60 (br s, 3H), 7.42 (br t, J=7.6 Hz, 1H), 7.34-7.29 (m, 1H), 7.18-7.12 (m, 2H), 4.41 (br d, J=14.5 Hz, 1H), 4.30 (br d, J=10.1 Hz, 1H), 3.72 (br dd, J=14.4, 6.5 Hz, 1H), 3.43 (s, 3H), 3.11-2.89 (m, 3H), 2.32-2.26 (m, 1H), 1.79-1.73 (m, 2H), 1.51-1.47 (m, 1H); LCMS (ES⁺) Method 2: m/z 587 (M+H)⁺, RT 1.35 min.

### Example 284: ((1S,6R,7R)-3-(3-(3-chloro-2-(pyrimidin-5-yl)pyridin-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

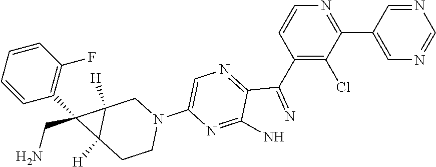 [SMILES: N=C(c1ccnc(-c2cncnc2)c1Cl)c1ncc(N2CC[C@@H]3[C@H](C2)[C@@]3(CN)c2ccccc2F)nc1N]

The above example was synthesized as reported for Example 2 using Intermediates 51, 44 and (2,3-dichloropyridin-4-yl)boronic acid) followed by the reaction with 5-(tributylstannyl)pyrimidine using the conditions of step 1 of Example 111; HBr (33% in AcOH) was used in step 2; Example 284 was obtained as an off-white powder (TFA salt; 3.1 mg, 96% pure, 13%). ¹H NMR (400 MHz, DMSO-d₆) δ 13.72 (s, 1H), 9.33 (s, 1H), 9.19 (s, 2H), 8.84-8.83 (m, 1H), 8.52 (s, 1H), 8.03-8.02 (m, 1H), 7.65 (br s, 3H), 7.49 (br t, J=7.1 Hz, 1H), 7.42-7.37 (m, 1H), 7.35-7.20 (m, 2H), 4.47-4.43 (m, 1H), 4.38-4.33 (m, 1H), 3.79-3.74 (m, 1H), 3.18-3.05 (m, 2H), 3.00-2.84 (m, 1H), 2.41-2.32 (m, 1H), 1.85-1.77 (m, 2H), 1.59-1.53 (m, 1H); LCMS (ES⁺) Method 2: m/z 528 (M+H)⁺, RT 1.11 min.

### Example 285: ((1S,6R,7R)-3-(3-(2-chloro-4-(2H-tetrazol-5-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

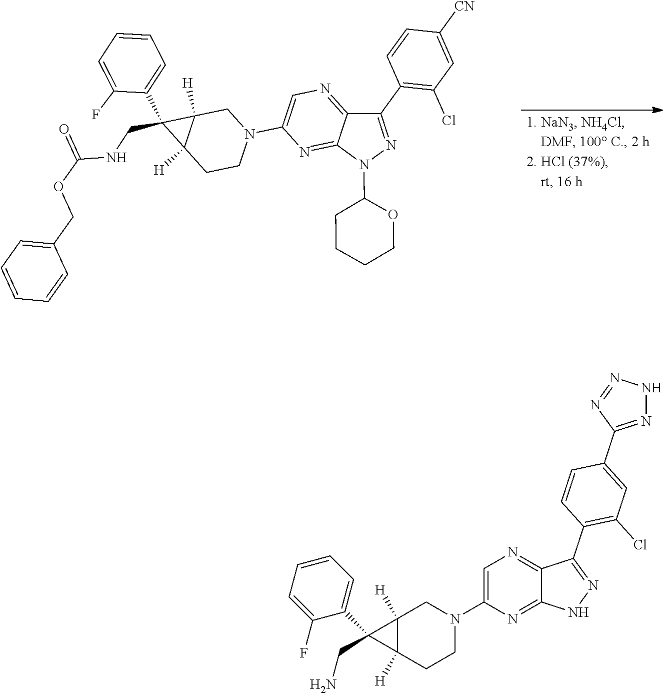 [SMILES: N#Cc1ccc(-c2nn(C3CCCCO3)c3nc(N4CC[C@@H]5[C@H](C4)[C@@]5(CNC(=O)OCc4ccccc4)c4ccccc4F)cnc23)c(Cl)c1 ; NC[C@]1(c2ccccc2F)[C@@H]2CCN(c3cnc4c(-c5ccc(-c6nn[nH]n6)cc5Cl)n[nH]c4n3)C[C@@H]21]

A sat. sol. of NH₄Cl (26.3 mg, 0.49 mmol), NaN₃ (31.9 mg, 0.49 mmol) and Example 245 (before deprotection step; 34.0 mg, 0.05 mmol) in DMF (1.0 mL) was stirred for 2 h at 100° C. After cooling the reaction mixture was diluted with DCM and the organic phase was washed with brine, dried over Na₂SO₄ and concentrated under reduced pressure to afford a residue which was purified by flash chromatography (0-100% EtOAc in petroleum ether) to obtain the title compound as a white powder (10.0 mg, 28%). LCMS (ES⁺) Method 2: m/z 735 (M+H)⁺, RT 2.33 min. This solid was dissolved in HCl (37%; 0.8 mL) and stirred at rt for 16 h. After volatiles removal in vacuo, the residue was purified by preparative HPLC (from 20% to 100% MeCN/H₂O+0.1% TFA) to obtain the title compound as a white solid (TFA salt; 2.5 mg, 36%). ¹H NMR (400 MHz, DMSO-d₆) δ 13.54 (s, 1H), 8.47 (s, 1H), 8.27 (br s, 1H), 8.16 (d, J=7.7 Hz, 1H), 8.08 (d, J=8.3 Hz, 1H), 7.66 (br s, 3H), 7.48 (br t, J=7.0 Hz, 1H), 7.42-7.36 (m, 1H), 7.25-7.19 (m, 2H), 4.46 (br d, J=14.7 Hz, 1H), 4.35 (br d, J=10.8 Hz, 1H), 3.77-3.72 (m, 1H), 3.50-3.39 (m, 1H), 3.19-3.04 (m, 2H), 2.99-2.92 (m, 1H), 2.38-2.31 (m, 1H), 1.85-1.78 (m, 2H), 1.57-1.54 (m, 1H); LCMS (ES⁺) Method 2: m/z 517 (M+H)⁺, RT 1.13 min.

### Example 286: 7-(aminomethyl)-3-(3-(4-chloro-2-methyl-2H-indazol-5-yl)-1H-pyrazolo[3,4-b]pyrazin-6-yl)-N,N-dimethyl-3-azabicyclo[4.1.0]heptane-7-carboxamide (Trifluoro Acetate Salt)

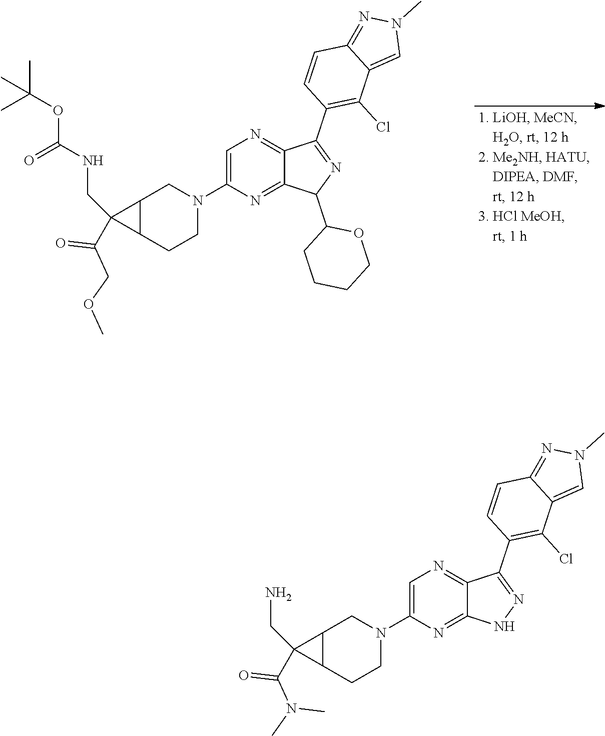 [SMILES: COCC(=O)C1(CNC(=O)OC(C)(C)C)C2CCN(c3cnc4c(n3)C(C3CCCCO3)N=C4c3ccc4nn(C)cc4c3Cl)CC21 ; CN(C)C(=O)C1(CN)C2CCN(c3cnc4c(-c5ccc6nn(C)cc6c5Cl)n[nH]c4n3)CC21]

### Step 1: 7-(((tert-butoxycarbonyl)amino)methyl)-3-(3-(4-chloro-2-methyl-2H-indazol-5-yl)-1-(tetrahydro-2H-pyran-2-yl)-1H-pyrazolo[3,4-b]pyrazin-6-yl)-3-azabicyclo[4.1.0]heptane-7-carboxylic acid

A solution of ethyl 7-(((tert-butoxycarbonyl)amino)methyl)-3-(3-(4-chloro-2-methyl-2H-indazol-5-yl)-1-(tetrahydro-2H-pyran-2-yl)-1H-pyrazolo[3,4-b]pyrazin-6-yl)-3-azabicyclo[4.1.0]heptane-7-carboxylate (prepared as reported for Example 2 using Intermediates 44, 45 and 95; 30.0 mg, 0.05 mmol) in a mixture of MeCN/H₂O (1/1; 2 mL) was treated with LiOH (2.2 mg, 0.09 mmol) and the reaction mixture was stirred at rt for 12 h. The solvent was removed under reduced pressure and the residue was suspended in a mixture of EtOAc/H₂O and neutralized dropwise with 1 N aq. HCl. The separated organic layer was dried over Na₂SO₄ and evaporated in vacuo to obtain the title compound as a yellow oil (28 mg, 97%) which was used in the next step without further purification. LCMS (ES⁺) Method 2: m/z 637 (M+H)⁺, RT 1.80 min.

### Step 2: tert-butyl ((3-(3-(4-chloro-2-methyl-2H-indazol-5-yl)-1-(tetrahydro-2H-pyran-2-yl)-1H-pyrazolo[3,4-b]pyrazin-6-yl)-7-(dimethylcarbamoyl)-3-azabicyclo[4.1.0]heptan-7-yl)methyl)carbamate

A solution of the material coming from previous step (19.0 mg, 0.03 mmol) in dry DMF (0.5 mL) was treated with DIPEA (0.02 mL, 0.12 mmol), dimethylamine hydrochloride (4.9 mg, 0.06 mmol) and HATU (13.6 mg, 0.04 mmol). The reaction mixture was stirred at rt for 12 h then EtOAc was added, and the mixture was washed with NH₄Cl sat. sol., NaHCO₃ sat. sol. and brine. The organic layer was dried over Na₂SO₄ and evaporated in vacuo to obtain the title compound as a yellow oil (19.0 mg, 96%) which was used in the next step without further purification. LCMS (ES⁺) Method 2: m/z 664 (M+H)⁺, RT 1.83 min.

### Step 3: 7-(aminomethyl)-3-(3-(4-chloro-2-methyl-2H-indazol-5-yl)-1H-pyrazolo[3,4-b]pyrazin-6-yl)-N,N-dimethyl-3-azabicyclo[4.1.0]heptane-7-carboxamide (Trifluoro Acetate Salt)

A solution of the residue coming from previous step (18.0 mg, 0.03 mmol) in MeOH (0.25 mL) was treated with HCl (4M in 1,4-dioxane; 0.5 mL) and stirred at rt for 1 h. After volatiles removal in vacuo the residue was purified by preparative HPLC (from 8% to 100% MeCN/H₂O+0.1% TFA) to obtain the title compound as a yellow solid (TFA salt; 7.0 mg, 54%). ¹H NMR (400 MHz, DMSO-d₆) δ 13.36 (br s, 1H), 8.56 (s, 1H), 8.40 (s, 1H), 7.85 (br s, 3H), 7.70-7.62 (m, 2H), 4.39-4.31 (m, 2H), 4.24 (s, 3H), 3.68-3.62 (m, 1H), 3.12-2.84 (m, 9H), 2.28-2.23 (m, 1H), 1.85-1.60 (m, 3H); LCMS (ES⁺) Method 2: m/z 480 (M+H)⁺, RT 0.84 min.

### Example 287: 7-(aminomethyl)-3-(3-(4-chloro-2-methyl-2H-indazol-5-yl)-1H-pyrazolo[3,4-b]pyrazin-6-yl)-N-methyl-3-azabicyclo[4.1.0]heptane-7-carboxamide (Trifluoro Acetate Salt)

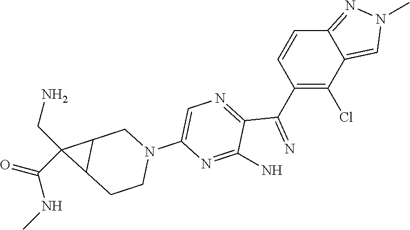 [SMILES: CNC(=O)C1(CN)C2CCN(c3cnc(C(=N)c4ccc5nn(C)cc5c4Cl)c(N)n3)CC21]

The above example was synthesized using the procedure reported for Example 286 using Intermediates 44, 45 and 95. Methanamine hydrochloride was used in step 2; Example 287 was obtained as a yellow powder (TFA salt; 7.0 mg, 55%). ¹H NMR (400 MHz, DMSO-d₆) δ 13.38 (br s, 1H), 8.56 (s, 1H), 8.37 (s, 1H), 7.76 (br s, 3H), 7.69-7.62 (m, 2H), 7.52 (br d, J=4.0 Hz, 1H), 4.35-4.20 (m, 5H), 3.65 (br dd, J=14.3, 5.7 Hz, 1H), 3.28-3.20 (m, 1H), 3.08-2.99 (m, 1H), 2.94-2.88 (m, 1H), 2.61 (br s, 3H), 2.27-2.21 (m, 1H), 2.08-1.99 (m, 1H), 1.84-1.81 (m, 1H), 1.69-1.63 (m, 1H); LCMS (ES⁺) Method 2: m/z 466 (M+H)⁺, RT 0.79 min.

### Example 291: ((1S,6R,7R)-3-(3-(4-chloro-2-methyl-2H-indazol-5-yl)-1H-pyrazolo[3,4-b]pyrazin-6-yl)-7-(3,5-difluorophenyl)-3-azabicyclo[4.1.0]heptan-7-yl)methanamine (Trifluoro Acetate Salt)

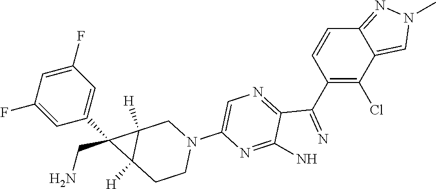 [SMILES: Cn1cc2c(Cl)c(C(=N)c3ncc(N4CC[C@@H]5[C@H](C4)[C@@]5(CN)c4cc(F)cc(F)c4)nc3N)ccc2n1]

The above example was synthesized using the procedure reported for Example 2 using Intermediates 44, 45 and 168; Example 291 was obtained as a yellow powder (TFA salt; 8.0 mg, 98% pure, 42%). ¹H NMR (400 MHz, DMSO-d₆) δ 13.38 (br s, 1H), 8.56 (s, 1H), 8.40 (s, 1H), 7.70-7.61 (br m, 5H), 7.26-7.21 (m, 2H), 7.19-7.14 (m, 1H), 4.44 (br d, J=14.5 Hz, 1H), 4.33 (br d, J=11.9 Hz, 1H), 4.24 (s, 3H), 3.67 (br dd, J=14.5, 6.6 Hz, 1H), 3.22-3.13 (m, 2H), 2.95-2.87 (m, 1H), 2.35-2.28 (m, 1H), 1.93-1.87 (m, 1H), 1.81-1.73 (m, 1H), 1.62-1.58 (m, 1H); LCMS (ES⁺) Method 2: m/z 521 (M+H)⁺, RT 1.16 min.

### Example 295: 3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-oxadiazol-5-ol (Trifluoro Acetate Salt)

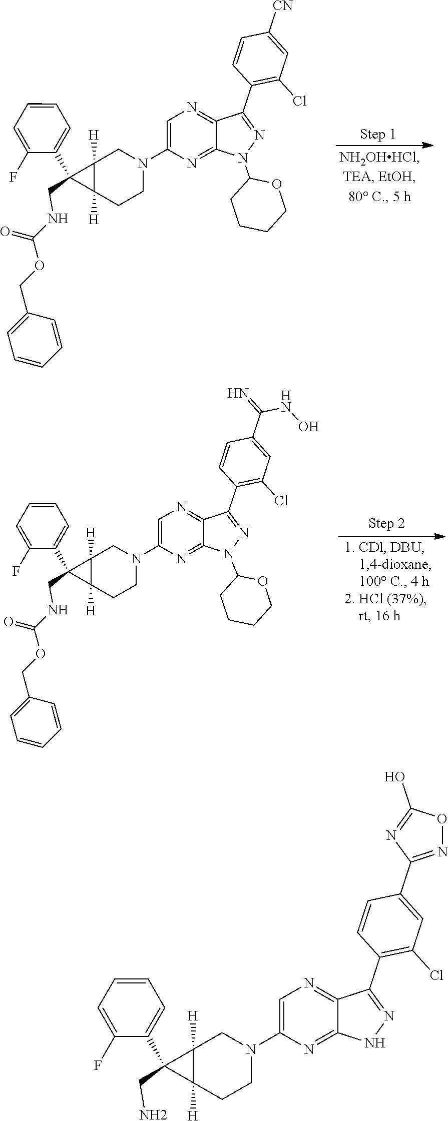 [SMILES: N#Cc1ccc(-c2nn(C3CCCCO3)c3nc(N4CC[C@@H]5[C@H](C4)[C@@]5(CNC(=O)OCc4ccccc4)c4ccccc4F)cnc23)c(Cl)c1 ; N=C(NO)c1ccc(-c2nn(C3CCCCO3)c3nc(N4CC[C@@H]5[C@H](C4)[C@@]5(CNC(=O)OCc4ccccc4)c4ccccc4F)cnc23)c(Cl)c1 ; NC[C@]1(c2ccccc2F)[C@@H]2CCN(c3cnc4c(-c5ccc(-c6noc(O)n6)cc5Cl)n[nH]c4n3)C[C@@H]21]

### Step 1: benzyl (((1S,6R,7R)-3-(3-(2-chloro-4-(N-hydroxycarbamimidoyl)phenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate

A solution of NH₂OH·HCl (4.7 mg, 0.07 mmol) and benzyl (((1S,6R,7R)-3-(3-(2-chloro-4-cyanophenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported for Example 2 using Intermediates 44, 51 and 3-chloro-4-(4,4,5,5-tetramethyl-1,3,2-dioxaborolan-2-yl)benzonitrile; 31.0 mg, 0.04 mmol) in EtOH (0.5 mL) was treated with TEA (9.3 uL, 0.07 mmol) and the resulting mixture was stirred for 5 h at 80° C. After cooling the reaction mixture was concentrated under reduced pressure and the residue was diluted with EtOAc, washed with brine, dried over Na₂SO₄, and evaporated in vacuo to obtain the title compound as a white powder (32.0 mg, 99%) which was used in the next step without further purification. LCMS (ES⁺) Method 2: m/z 725 (M+H)⁺, RT 1.93 min.

### Step 2: benzyl (((1S,6R,7R)-3-(3-(2-chloro-4-(5-oxo-4,5-dihydro-1,2,4-oxadiazol-3-yl)phenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate

A solution of DBU (8.9 uL, 0.06 mmol), CDI (9.6 mg, 0.06 mmol) and the product coming from the previous step (32.0 mg, 0.05 mmol) in 1,4-dioxane (0.6 mL) was stirred for 4 h at 100° C. After cooling the reaction mixture was diluted with EtOAc, washed with brine, dried over Na₂SO₄, and evaporated in vacuo to obtain a residue which was purified by flash chromatography (0-80% EtOAc (+5% MeOH) in petroleum ether) to obtain the title compound as a yellow solid (21 mg, 61%). LCMS (ES⁺) Method 2: m/z 751 (M+H)⁺, RT 2.44 min. This crude (7.0 mg, 0.01 mmol) was dissolved in HCl (37%; 0.4 mL, 0.01 mmol) and stirred at rt for 16 h. After volatiles removal in vacuo the residue was purified by preparative HPLC (from 13% to 100% MeCN/H₂O+0.1% TFA) to obtain the title compound as a yellow solid (TFA salt; 2.0 mg, 40%). LCMS (ES⁺) Method 2: m/z 533 (M+H)⁺, RT 1.16 min.

### Example 296: ((1S,6R,7R)-3-(3-(2-chloro-4-(1H-pyrazol-5-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

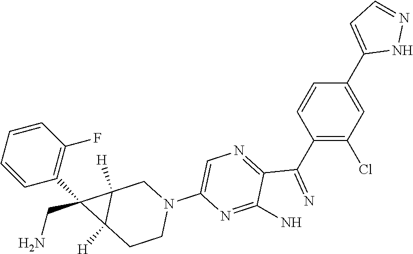 [SMILES: N=C(c1ccc(-c2ccn[nH]2)cc1Cl)c1ncc(N2CC[C@@H]3[C@H](C2)[C@@]3(CN)c2ccccc2F)nc1N]

The above example was synthesized as reported for Example 2 using Intermediates 51, 44 and (4-bromo-2-chlorophenyl)boronic acid) followed by the reaction with 5-(4,4,5,5-tetramethyl-1,3,2-dioxaborolan-2-yl)-1-((2-(trimethylsilyl)ethoxy)methyl)-1H-pyrazole using the conditions of step 1 (Pd(dppf)Cl₂·DCM as catalyst) of Example 111; HBr (33% in AcOH) was used in step 2; Example 296 was obtained as a white powder (TFA salt; 2.5 mg, 52%). LCMS (ES⁺) Method 2: m/z 515 (M+H)⁺, RT 1.18 min.

### Example 297: ((1S,6R,7R)-3-(3-(2-chloro-4-(1-methyl-1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

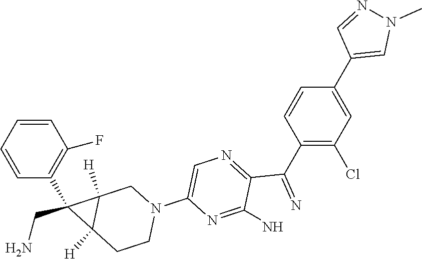 [SMILES: Cn1cc(-c2ccc(C(=N)c3ncc(N4CC[C@@H]5[C@H](C4)[C@@]5(CN)c4ccccc4F)nc3N)c(Cl)c2)cn1]

The above example was synthesized as reported for Example 2 using Intermediates 51, 44 and (4-bromo-2-chlorophenyl)boronic acid) followed by the reaction with 1-methyl-4-(4,4,5,5-tetramethyl-1,3,2-dioxaborolan-2-yl)-1H-pyrazole using the conditions of step 1 (Pd(dppf)Cl₂·DCM as catalyst) of Example 111; HBr (33% in AcOH) was used in step 2; Example 297 was obtained as a white powder (TFA salt; 3.3 mg, 21%). ¹H NMR (400 MHz, DMSO-d₆) δ 13.36 (br s, 1H), 8.43 (s, 1H), 8.31 (s, 1H), 8.01 (s, 1H), 7.83 (d, J=1.5 Hz, 1H), 7.77 (d, J=8.1 Hz, 1H), 7.70-7.65 (br m, 4H), 7.49 (br t, J=7.8 Hz, 1H), 7.41-7.36 (m, 1H), 7.25-7.19 (m, 2H), 4.45 (br d, J=13.8 Hz, 1H), 4.34 (br d, J=9.7 Hz, 1H), 3.89 (s, 3H), 3.75-3.70 (m, 1H), 3.18-3.05 (m, 2H), 2.97-2.90 (m, 1H), 2.38-2.31 (m, 1H), 1.84-1.76 (m, 2H), 1.57-1.53 (m, 1H); LCMS (ES⁺) Method 2: m/z 529 (M+H)⁺, RT 1.23 min.

### Example 306: 6-(6-((1S,6R,7R)-7-(aminomethyl)-7-(3,5-difluorophenyl)-3-azabicyclo[4.1.0]heptan-3-yl)-1H-pyrazolo[3,4-b]pyrazin-3-yl)-5-fluoroisoquinolin-1(2H)-one (Trifluoro Acetate Salt)

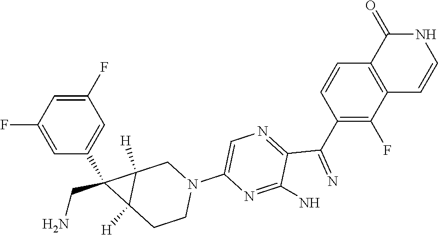 [SMILES: N=C(c1ccc2c(=O)[nH]ccc2c1F)c1ncc(N2CC[C@@H]3[C@H](C2)[C@@]3(CN)c2cc(F)cc(F)c2)nc1N]

The above example was synthesized using the procedure reported for Example 2 using Intermediates 44 and 168; HBr (33% in AcOH) was used in the final deprotection step; Example 306 was obtained as a yellow powder (TFA salt; 9.5 mg, 52%). ¹H NMR (400 MHz, DMSO-d₆) δ 13.58 (s, 1H), 11.53 (br s, 1H), 8.47 (s, 1H), 8.26-8.22 (m, 1H), 8.13 (d, J=8.6 Hz, 1H), 7.62 (br s, 3H), 7.33 (brt, J=6.7 Hz, 1H), 7.24 (br d, J=6.6 Hz, 2H), 7.18-7.14 (m, 1H), 6.70 (d, J=7.0 Hz, 1H), 4.44 (br d, J=15.1 Hz, 1H), 4.35 (br d, J=10.3 Hz, 1H), 3.73-3.68 (m, 1H), 3.20-3.09 (m, 2H), 2.96-2.90 (m, 1H), 2.36-2.28 (m, 1H), 1.94-1.87 (m, 1H), 1.79-1.74 (m, 1H), 1.62-1.58 (m, 1H); LCMS (ES⁺) Method 2: m/z 518 (M+H)⁺, RT 1.12 min.

### Example 307: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methylpicolinamide (Trifluoro Acetate Salt)

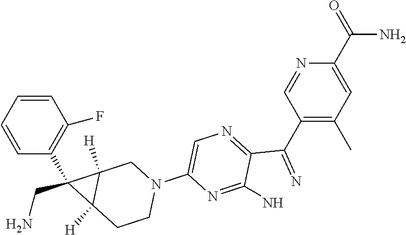 [SMILES: Cc1cc(C(N)=O)ncc1C(=N)c1ncc(N2CC[C@@H]3[C@H](C2)[C@@]3(CN)c2ccccc2F)nc1N]

The above example was synthesized using the procedure reported for Example 276 using Intermediates 44, 51 and (6-(methoxycarbonyl)-4-methylpyridin-3-yl)boronic acid (prepared as reported for the synthesis of Intermediate 15); HBr (33% in AcOH) was used in the final deprotection step; Example 307 was obtained as a yellow powder (TFA salt; 1.4 mg, 98% pure, 15%). LCMS (ES⁺) Method 2: m/z 473 (M+H)⁺, RT 1.04 min.

### Example 308: ((1S,6R,7R)-7-(3,5-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine (Trifluoro Acetate Salt)

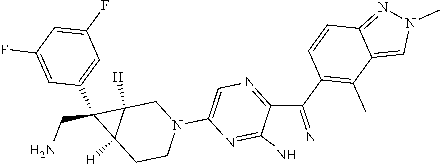 [SMILES: Cc1c(C(=N)c2ncc(N3CC[C@@H]4[C@H](C3)[C@@]4(CN)c3cc(F)cc(F)c3)nc2N)ccc2nn(C)cc12]

The above example was synthesized using the procedure reported for Example 2 using Intermediates 44, 53 and 168; HBr (33% in AcOH) instead of HCl (37%) was used in the final deprotection step; Example 308 was obtained as a yellow powder (TFA salt; 9.5 mg, 95% pure, 50%). ¹H NMR (400 MHz, DMSO-d₆) δ 12.99 (br s, 1H), 8.31 (s, 1H), 8.20 (s, 1H), 7.55 (d, J=9.0 Hz, 1H), 7.47 (br s, 3H), 7.32 (d, J=9.0 Hz, 1H), 7.05 (br d, J=6.8 Hz, 2H), 6.97 (br t, J=9.3 Hz, 1H), 4.26 (br d, J=14.3 Hz, 1H), 4.15 (br d, J=9.8 Hz, 1H), 4.01 (s, 3H), 3.50-3.45 (m, 1H), 3.14-3.08 (m, 1H), 3.01-2.96 (m, 1H), 2.75-2.67 (m, 1H), 2.46 (s, 3H), 2.15-2.09 (m, 1H), 1.74-1.68 (m, 1H), 1.62-1.53 (m, 1H), 1.42-1.38 (m, 1H); LCMS (ES⁺) Method 2: m/z 501 (M+H)⁺, RT 1.15 min.

### Example 310: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyanobenzamide (Trifluoro Acetate Salt)

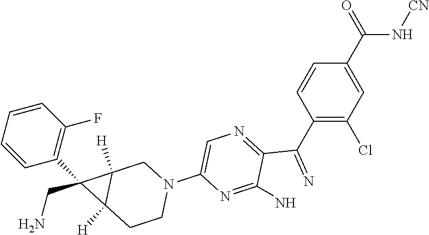 [SMILES: N#CNC(=O)c1ccc(C(=N)c2ncc(N3CC[C@@H]4[C@H](C3)[C@@]4(CN)c3ccccc3F)nc2N)c(Cl)c1]

The above example was synthesized using the procedure reported for Example 273 using the same starting material but cyanamide in step 2 and HBr (33% in AcOH) in step 3; Example 310 was obtained as a yellow powder (TFA salt; 0.9 mg, 21%). LCMS (ES⁺) Method 2: m/z 517 (M+H)⁺, RT 1.12 min.

### Example 313: 4′-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3′-chloro-3,5-difluoro-[1,1′-biphenyl]-4-ol (Trifluoro Acetate Salt)

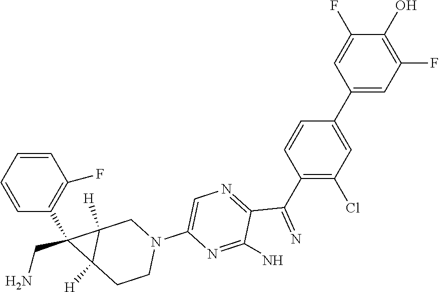 [SMILES: N=C(c1ccc(-c2cc(F)c(O)c(F)c2)cc1Cl)c1ncc(N2CC[C@@H]3[C@H](C2)[C@@]3(CN)c2ccccc2F)nc1N]

The above example was synthesized as reported for Example 2 using Intermediates 51, 44 and (4-bromo-2-chlorophenyl)boronic acid) followed by the reaction with 2,6-difluoro-4-(4,4,5,5-tetramethyl-1,3,2-dioxaborolan-2-yl)phenol using the conditions of step 1 (Pd(dppf)Cl₂·DCM as catalyst) of Example 111. Example 313 was obtained as a yellow powder (TFA salt; 2.6 mg, 9%). ¹H NMR (400 MHz, DMSO-d₆) δ 13.41 (br s, 1H), 10.49 (s, 1H), 8.45 (s, 1H), 7.93 (d, J=1.5 Hz, 1H), 7.87 (d, J=8.1 Hz, 1H), 7.80-7.78 (m, 1H), 7.66 (br s, 3H), 7.58 (br d, J=9.7 Hz, 2H), 7.51-7.47 (m, 1H), 7.42-7.36 (m, 1H), 7.25-7.20 (m, 2H), 4.46 (br d, J=13.8 Hz, 1H), 4.34 (br d, J=10.1 Hz, 1H), 3.73 (dd, J=13.8, 6.1 Hz, 1H), 3.19-3.05 (m, 2H), 2.98-2.91 (m, 1H), 2.40-2.37 (m, 1H), 1.84-1.78 (m, 2H), 1.57-1.53 (m, 1H); LCMS (ES⁺) Method 2: m/z 577 (M+H)⁺, RT 1.33 min.

### Example 315: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-fluoro-2-methylisoquinolin-1(2H)-one (Trifluoro Acetate Salt)

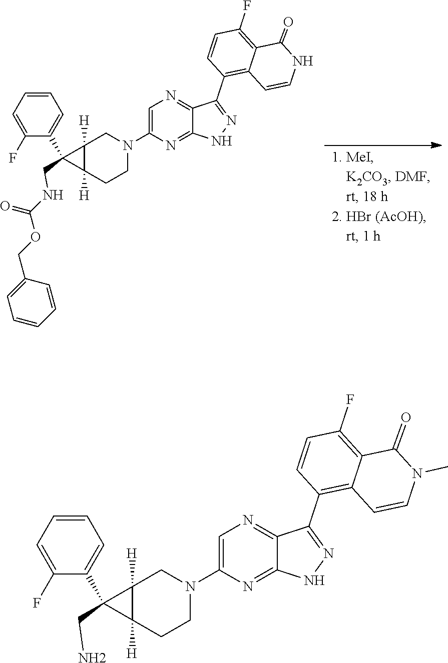 [SMILES: O=C(NC[C@]1(c2ccccc2F)[C@@H]2CCN(c3cnc4c(-c5ccc(F)c6c(=O)[nH]ccc56)n[nH]c4n3)C[C@@H]21)OCc1ccccc1 ; Cn1ccc2c(-c3n[nH]c4nc(N5CC[C@@H]6[C@H](C5)[C@@]6(CN)c5ccccc5F)cnc34)ccc(F)c2c1=O]

A solution of benzyl (((1S,6R,7R)-3-(3-(8-fluoro-1-oxo-1,2-dihydroisoquinolin-5-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as reported in the synthesis of Example 314; 20.0 mg, 0.03 mmol) in DMF (0.25 mL) was treated with K₂CO₃ (8.5 mg, 0.06 mmol) and Mel (0.01 mL, 0.14 mmol). The reaction mixture was stirred at rt for 18 h, then diluted with EtOAc and washed with HCl (1N aq. sol.), NaHCO₃ sat. sol. and brine. The organic phase was dried over Na₂SO₄ and evaporated in vacuo to obtain the title compound as a yellow oil (20.0 mg, 99%) which was used in the next step without further purification. LCMS (ES⁺) Method 2: m/z 732 (M+H)⁺, RT 2.34 min. A solution of this crude (20.0 mg, 0.03 mmol) dissolved in HBr (33% in AcOH; 0.5 mL, 0.03 mmol) was stirred at rt for 1 h. After volatiles removal in vacuo the residue was purified by preparative HPLC (from 15% to 100% MeCN/H₂O+0.1% TFA) to obtain the title compound as a yellow solid (TFA salt; 1.5 mg, 95% pure, 10%). LCMS (ES⁺) Method 2: m/z 514 (M+H)⁺, RT 1.10 min.

### Example 316: 6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-fluoro-2-methylisoquinolin-1(2H)-one (Trifluoro Acetate Salt)

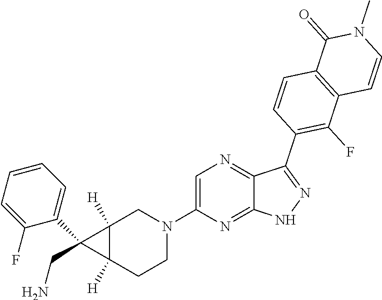 [SMILES: Cn1ccc2c(F)c(-c3n[nH]c4nc(N5CC[C@@H]6[C@H](C5)[C@@]6(CN)c5ccccc5F)cnc34)ccc2c1=O]

The above example was synthesized using the procedure reported for Example 315 using benzyl (((1S,6R,7R)-3-(3-(5-fluoro-1-oxo-1,2-dihydroisoquinolin-6-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate prepared as reported in Example 2 using Intermediates 44, 51 and 5-fluoro-2-methyl-6-(4,4,5,5-tetramethyl-1,3,2-dioxaborolan-2-yl)isoquinolin-1(2H)-one (prepared as reported for the synthesis of Intermediate 15); Example 316 was obtained as a yellow powder (TFA salt; 3.1 mg, 34%). ¹H NMR (400 MHz, DMSO-d₆) δ 13.60 (br s, 1H), 8.51 (s, 1H), 7.26 (t, J=7.0 Hz, 1H), 7.17 (d, J=7.9 Hz, 1H), 7.68 (br s, 3H), 7.64 (d, J=7.7 Hz, 1H), 7.49 (br t, J=7.6 Hz, 1H), 7.41-7.36 (m, 1H), 7.25-7.19 (m, 2H), 6.77 (d, J=7.5 Hz, 1H), 4.46 (br d, J=14.0 Hz, 1H), 4.35 (br d, J=9.4 Hz, 1H), 3.76 (dd, J=14.3, 6.6 Hz, 1H), 3.57 (s, 3H), 3.18-3.04 (m, 2H), 3.00-2.94 (m, 1H), 2.40-2.32 (m, 1H), 1.85-1.77 (m, 2H), 1.57-1.53 (m, 1H); LCMS (ES⁺) Method 2: m/z 514 (M+H)⁺, RT 1.12 min.

### Example 323: ((1S,6R,7R)-3-(3-(3-chloro-3′,5′-difluoro-[1,1′-biphenyl]-4-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

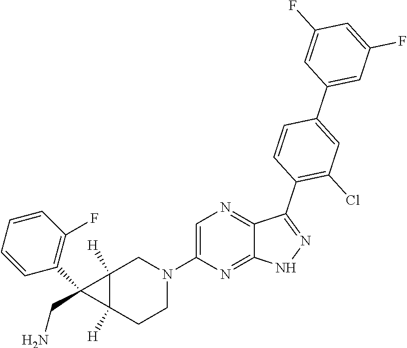 [SMILES: NC[C@]1(c2ccccc2F)[C@@H]2CCN(c3cnc4c(-c5ccc(-c6cc(F)cc(F)c6)cc5Cl)n[nH]c4n3)C[C@@H]21]

The above example was synthesized as reported for Example 2 using Intermediates 51, 44 and and (4-bromo-2-chlorophenyl)boronic acid) followed by the reaction with (3,5-difluorophenyl)boronic acid using the conditions of step 1 (Pd(dppf)Cl₂·DCM as catalyst) of Example 111; HBr (33% in AcOH) was used in the final deprotection step; Example 323 was obtained as a yellow powder (TFA salt; 5.2 mg, 17%). LCMS (ES⁺) Method 2: m/z 561 (M+H)⁺, RT 1.61 min.

### Example 324: 2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2H-indazol-2-yl)acetamide (Trifluoro Acetate Salt)

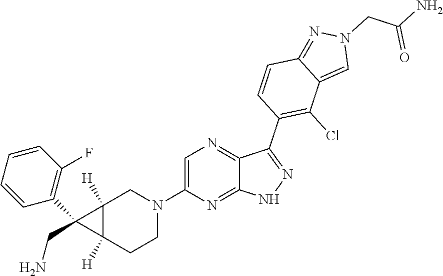 [SMILES: NC[C@]1(c2ccccc2F)[C@@H]2CCN(c3cnc4c(-c5ccc6nn(CC(N)=O)cc6c5Cl)n[nH]c4n3)C[C@@H]21]

The above example was synthesized using the procedure reported for Example 273 using methyl 2-(5-(6-((1S,6R,7R)-7-((((benzyloxy)carbonyl)amino)methyl)-7-(2-fluorophenyl)-3-azabicyclo[4.1.0]heptan-3-yl)-1-(tetrahydro-2H-pyran-2-yl)-1H-pyrazolo[3,4-b]pyrazin-3-yl)-4-chloro-2H-indazol-2-yl)acetate (prepared as reported for the synthesis of Example 2 using Intermediates 44, 51 and methyl 2-(4-chloro-5-(4,4,5,5-tetramethyl-1,3,2-dioxaborolan-2-yl)-2H-indazol-2-yl)acetate); NH₄Cl was used in step 2 and HBr (33% in AcOH) was used in the final deprotection step; Example 324 was obtained as a white powder (TFA salt; 3.5 mg, 38%). LCMS (ES⁺) Method 2: m/z 546 (M+H)⁺, RT 1.11 min.

### Example 328: 2-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)acetamide (Trifluoro Acetate Salt)

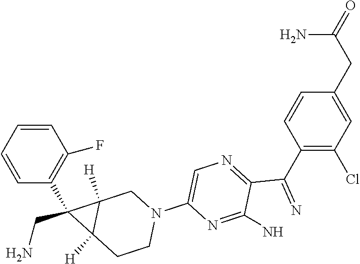 [SMILES: N=C(c1ccc(CC(N)=O)cc1Cl)c1ncc(N2CC[C@@H]3[C@H](C2)[C@@]3(CN)c2ccccc2F)nc1N]

The above example was synthesized using the procedure reported for Example 276 using methyl 2-(4-(6-((1S,6R,7R)-7-((((benzyloxy)carbonyl)amino)methyl)-7-(2-fluorophenyl)-3-azabicyclo[4.1.0]heptan-3-yl)-1-(tetrahydro-2H-pyran-2-yl)-1H-pyrazolo[3,4-b]pyrazin-3-yl)-3-chlorophenyl)acetate (prepared as reported for the synthesis of Example 2 using Intermediates 44, 51 and methyl 2-(3-chloro-4-(4,4,5,5-tetramethyl-1,3,2-dioxaborolan-2-yl)phenyl)acetate); HBr (33% in AcOH) was used in the final deprotection step; Example 328 was obtained as a yellow powder (TFA salt; 2.8 mg, 20%). LCMS (ES⁺) Method 2: m/z 506 (M+H)⁺, RT 1.03 min.

### Example 334: ((1S,6R,7R)-7-(2-fluorophenyl)-3-(3-((2-fluorophenyl)ethynyl)-1H-pyrazolo[3,4-b]pyrazin-6-yl)-3-azabicyclo[4.1.0]heptan-7-yl)methanamine (Trifluoro Acetate Salt)

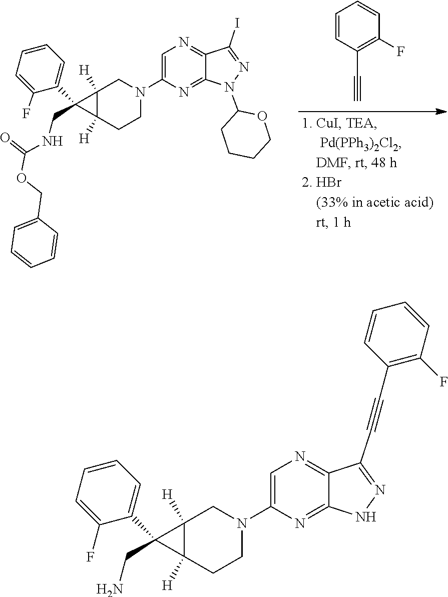

A solution of CuI (0.7 mg, 0.004 mmol), TEA (25.5 uL, 0.18 mmol), 1-ethynyl-2-fluorobenzene (8.8 mg, 0.07 mmol), Pd(PPh₃)₂Cl₂ (2.6 mg, 0.004 mmol) and benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared using Intermediates 44 and 51; 25.0 mg, 0.04 mmol) in degassed DMF (1 mL) was stirred at rt for 48 h. The reaction mixture was diluted with EtOAc, washed with brine, dried over Na₂SO₄, filtered, and concentrated in vacuo to afford a residue which was purified by flash chromatography (0-50% EtOAc in petroleum ether) to obtain the title compound as a yellow solid (17 mg, 69%). LCMS (ES⁺) Method 2 m/z 675 (M+H)⁺, RT 2.59 min. The residue (17.0 mg, 0.03 mmol) was treated with HBr (33% in AcOH; 0.6 mL, 0.03 mmol) and the mixture was stirred at rt for 1 h. After volatiles removal in vacuo the residue was purified by preparative HPLC (from 25% to 100% MeCN/H₂O+0.1% TFA) to obtain the title compound as a white solid (TFA salt; 5.6 mg, 49%). LCMS (ES⁺) Method 2: m/z 457 (M+H)⁺, RT 1.34 min.

### Example 336: 6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-2-methylisoquinolin-1(2H)-one (Trifluoro Acetate Salt)

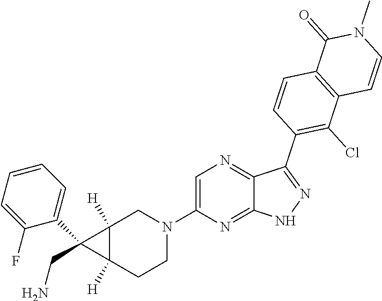 [SMILES: Cn1ccc2c(Cl)c(-c3n[nH]c4nc(N5CC[C@@H]6[C@H](C5)[C@@]6(CN)c5ccccc5F)cnc34)ccc2c1=O]

The above example was synthesized using the procedure reported for Example 315 using benzyl (((1S,6R,7R)-3-(3-(5-chloro-1-oxo-1,2-dihydroisoquinolin-6-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate prepared as reported in Example 2 using Intermediates 44, 51 and 5-chloro-2-methyl-6-(4,4,5,5-tetramethyl-1,3,2-dioxaborolan-2-yl)isoquinolin-1(2H)-one (prepared as reported for the synthesis of Intermediate 15); HBr (33% in AcOH) was used in the final deprotection step; Example 336 was obtained as a yellow powder (TFA salt; 1.3 mg, 9%). ¹H NMR (400 MHz, DMSO-d₆) δ 13.54 (br s, 1H), 8.45 (s, 1H), 8.32 (d, J=8.3 Hz, 1H), 7.84 (d, J=8.3 Hz, 1H), 7.71 (d, J=7.7 Hz, 1H), 7.67 (br s, 3H), 7.49 (br t, J=7.9 Hz, 1H), 7.41-7.36 (m, 1H), 7.25-7.19 (m, 2H), 6.91 (d, J=7.5 Hz, 1H), 4.45 (br d, J=14.7 Hz, 1H), 4.34 (br d, J=10.3 Hz, 1H), 3.74 (br dd, J=14.4, 6.5 Hz, 1H), 3.58 (s, 3H), 3.20-3.05 (m, 2H), 2.99-2.90 (m, 1H), 2.39-2.31 (m, 1H), 1.84-1.77 (m, 2H), 1.57-1.53 (m, 1H); LCMS (ES⁺) Method 2: m/z 530 (M+H)⁺, RT 1.16 min.

### Example 342: ((1S,6R,7R)-3-(5-((3-chloro-2-(1H-pyrazol-1-yl)pyridin-4-yl)thio)-1H-imidazo[4,5-b]pyrazin-2-yl)-7-(2-fluorophenyl)-3-azabicyclo[4.1.0]heptan-7-yl)methanamine (trifluoro acetate salt)

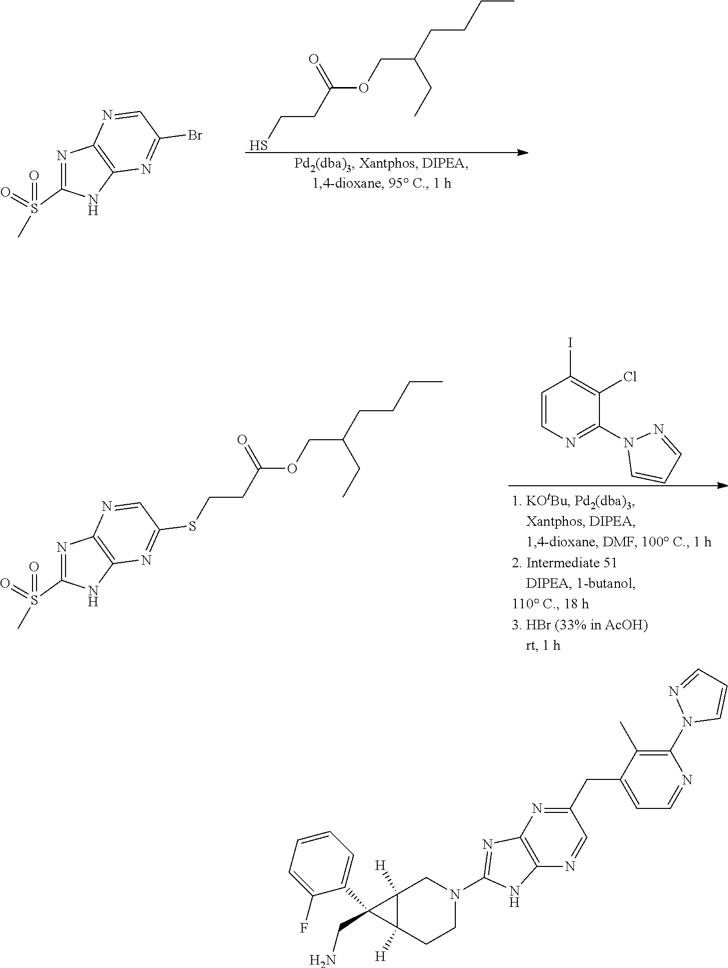 [SMILES: CS(=O)(=O)c1nc2ncc(Br)nc2[nH]1 ; CCCCC(CC)COC(=O)CCS ; CCCCC(CC)COC(=O)CCSc1cnc2nc(S(C)(=O)=O)[nH]c2n1 ; Clc1c(I)ccnc1-n1cccn1 ; Cc1c(Cc2cnc3[nH]c(N4CC[C@@H]5[C@H](C4)[C@@]5(CN)c4ccccc4F)nc3n2)ccnc1-n1cccn1]

### Step 1: 2-ethylhexyl 3-((2-(methylsulfonyl)-1H-imidazo[4,5-b]pyrazin-6-yl)thio)propanoate

A pressure tube was charged with 2-ethylhexyl 3-mercaptopropanoate (0.45 mL, 1.98 mmol), 6-bromo-2-(methylsulfonyl)-1H-imidazo[4,5-b]pyrazine (0.5 g, 1.8 mmol), XantPhos (52.2 mg, 0.09 mmol), Pd₂(dba)₃ (41.3 mg, 0.05 mmol) and DIPEA (0.64 mL, 3.61 mmol) in 1,4-dioxane (7.6 mL). The reaction mixture was degassed and heated at 95° C. for 1 h. After cooling the mixture was concentrated in vacuo to afford a residue which was purified by flash chromatography (10-100% EtOAc in petroleum ether) to obtain the title compound as a yellow solid (530 mg, 71%). LCMS (ES⁺) Method 2: m/z 415 (M+H)⁺, RT 2.17 min.

### Step 2: 5-((3-chloro-2-(1H-pyrazol-1-yl)pyridin-4-yl)thio)-2-(methylsulfonyl)-1H-imidazo[4,5-b]pyrazine

A solution of 2-ethylhexyl 3-((2-(methylsulfonyl)-1H-imidazo[4,5-b]pyrazin-6-yl)thio)propanoate (200.0 mg, 0.48 mmol), 3-chloro-4-iodo-2-(1H-pyrazol-1-yl)pyridine (191.6 mg, 0.63 mmol), XantPhos (13.9 mg, 0.02 mmol) and Pd₂(dba)₃ (11.0 mg, 0.01 mmol) in 1,4-dioxane (3.3 mL) and DMF (1.5 mL) was treated with tBuOK (0.96 mL, 0.96 mmol in THF) and DIPEA (0.17 mL, 0.96 mmol) and the mixture was stirred at 100° C. for 1 h. After cooling the reaction mixture was concentrated in vacuo to afford a residue which was purified by flash chromatography (C18; 10-100% MeCN/H₂O+0.1% TFA) to obtain the title compound as a yellow solid (50 mg, 25%). LCMS (ES⁺) Method 2: m/z 408 (M+H)⁺, RT 1.30 min. A solution of this residue (25.0 mg, 0.06 mmol), benzyl (((1S,6R,7R)-7-(2-fluorophenyl)-3-azabicyclo[4.1.0]heptan-7-yl)methyl)carbamate hydrochloride (28.8 mg, 0.07 mmol) and DIPEA (0.05 mL, 0.31 mmol) in 1-BuOH (0.61 mL) was heated at 110° C. for 18 h. After cooling the reaction mixture was concentrated in vacuo to afford a crude which was purified by flash chromatography (0-10% MeOH in DCM) to obtain the desired compound as a yellow solid (6 mg) which was dissolved in HBr (33% in AcOH; 0.5 mL). The reaction mixture was stirred at rt for 1 h. After volatiles removal in vacuo the residue was purified by preparative HPLC (from 15% to 100% MeCN/H₂O+0.1% TFA) to obtain the title compound as a yellow solid (TFA salt; 2.0 mg, 5%). LCMS (ES⁺) Method 2: m/z 548 (M+H)⁺, RT 1.13 min.

### Example 343: 5-(6-((1S,6R,7R)-7-(aminomethyl)-7-(2-fluorophenyl)-3-azabicyclo[4.1.0]heptan-3-yl)-5-methyl-4-oxo-4,5-dihydro-1H-pyrazolo[3,4-d]pyrimidin-3-yl)quinoline-8-carboxamide (trifluoro acetate salt)

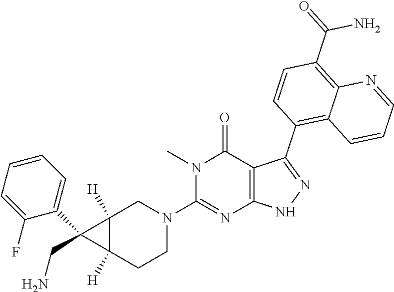 [SMILES: Cn1c(N2CC[C@@H]3[C@H](C2)[C@@]3(CN)c2ccccc2F)nc2[nH]nc(-c3ccc(C(N)=O)c4ncccc34)c2c1=O]

The above example was synthesized using the procedure reported for Example 62 (DMSO instead of NMP was used in step 1) using Intermediates 47, 51 and Intermediate 161; Example 343 was obtained as a yellow powder (TFA salt; 4.5 mg, 30%). LCMS (ES⁺) Method 2: m/z 539 (M+H)⁺, RT 0.99 min.

### Example 344: 6-((1S,6R,7R)-7-(aminomethyl)-7-(3,5-difluorophenyl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one (Trifluoro Acetate Salt)

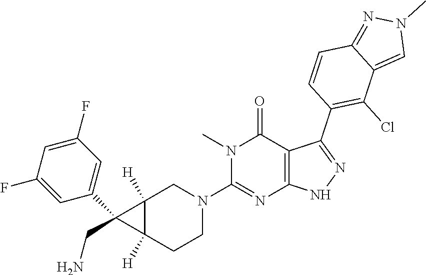 [SMILES: Cn1cc2c(Cl)c(-c3n[nH]c4nc(N5CC[C@@H]6[C@H](C5)[C@@]6(CN)c5cc(F)cc(F)c5)n(C)c(=O)c34)ccc2n1]

The above example was synthesized using the procedure reported for Example 62 using Intermediates 45, 47 and 168 (DMSO instead of NMP was used in step 1); HBr (33% in AcOH) was used in the final deprotection step; Example 344 was obtained as a white powder (TFA salt; 2.7 mg, 98% pure, 37%). ¹H NMR (400 MHz, DMSO-d₆) δ 13.54 (br s, 1H), 8.54 (s, 1H), 7.69 (br s, 3H), 7.61 (br d, J=7.2 Hz, 1H), 7.32 (d, J=8.8 Hz, 1H), 7.23 (d, J=6.8 Hz, 2H), 7.19-7.14 (m, 1H), 4.24 (s, 3H), 3.82-3.77 (m, 1H), 3.65-3.60 (m, 1H), 3.57-3.51 (m, 2H), 3.43-3.40 (m, 1H), 3.35 (s, 3H), 2.65-2.56 (m, 1H), 2.29-2.20 (m, 1H), 2.01-1.95 (m, 1H), 1.83-1.78 (m, 1H), 1.68-1.65 (m, 1H); LCMS (ES⁺) Method 2: m/z 551 (M+H)⁺, RT 1.13 min.

### Example 356: 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyano-N-methylbenzamide (Trifluoro Acetate Salt)

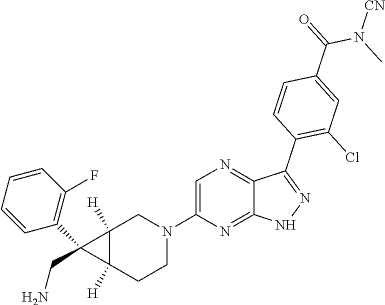 [SMILES: CN(C#N)C(=O)c1ccc(-c2n[nH]c3nc(N4CC[C@@H]5[C@H](C4)[C@@]5(CN)c4ccccc4F)cnc23)c(Cl)c1]

The above example was synthesized using the procedure reported for Example 273 (N-methylcyanamide 1M in DCM was used in step 2, and HBr (33% in AcOH) was used in step 3) using Intermediates 44, 51 and methyl 3-chloro-4-(4,4,5,5-tetramethyl-1,3,2-dioxaborolan-2-yl)benzoate (prepared as reported for the synthesis of Intermediate 15); Example 356 was obtained as a white powder (TFA salt; 0.7 mg, 14%). LCMS (ES⁺) Method 2: m/z 531 (M+H)⁺, RT 1.29 min.

### Example 366: ((1S,6R,7R)-3-(3-(3,3-dimethylbut-1-yn-1-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

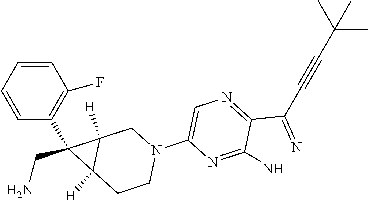 [SMILES: CC(C)(C)C#CC(=N)c1ncc(N2CC[C@@H]3[C@H](C2)[C@@]3(CN)c2ccccc2F)nc1N]

The above example was synthesized using the procedure reported for Example 334 using 3,3-dimethylbut-1-yne in step 1; Example 366 was obtained as a white powder (TFA salt; 4.1 mg, 42%). LCMS (ES⁺) Method 2: m/z 419 (M+H)⁺, RT 1.49 min.

### Example 369: ((1S,6R,7R)-3-(3-(4-chloro-2-methyl-2H-indazol-5-yl)-1H-pyrazolo[3,4-b]pyrazin-6-yl)-7-(2,5-difluorophenyl)-3-azabicyclo[4.1.0]heptan-7-yl)methanamine (Trifluoro Acetate Salt)

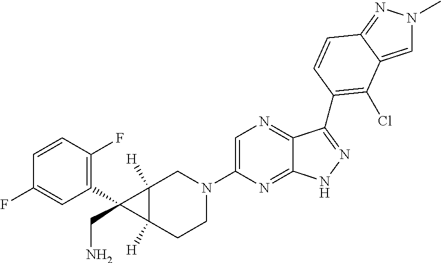 [SMILES: Cn1cc2c(Cl)c(-c3n[nH]c4nc(N5CC[C@@H]6[C@H](C5)[C@@]6(CN)c5cc(F)ccc5F)cnc34)ccc2n1]

The above example was synthesized using the procedure reported for Example 2 using Intermediates 44, 102 and 45; HBr (33% in AcOH) was used in the final deprotection step; Example 369 was obtained as a yellow powder (TFA salt; 3.3 mg, 15%). ¹H NMR (400 MHz, DMSO-d₆) δ 13.36 (br s, 1H), 8.56 (s, 1H), 8.43 (s, 1H), 7.70-7.63 (br m, 5H), 7.34-7.20 (m, 3H), 4.45 (br d, J=14.0 Hz, 1H), 4.33 (br d, J=12.9 Hz, 1H), 4.24 (s, 3H), 3.73-3.67 (m, 1H), 3.18-3.06 (m, 2H), 2.97-2.90 (m, 1H), 2.36-2.30 (m, 1H), 1.88-1.77 (m, 2H), 1.61-1.57 (m, 1H); LCMS (ES⁺) Method 2: m/z 521 (M+H)⁺, RT 1.20 min.

### Example 377: ((1S,6R,7R)-3-(3-(2-chloro-4-(5-methyl-1,2,4-oxadiazol-3-yl)phenyl)-1H-pyrazolo[3,4-b]pyrazin-6-yl)-7-(2-fluorophenyl)-3-azabicyclo[4.1.0]heptan-7-yl)methanamine (trifluoro acetate salt)

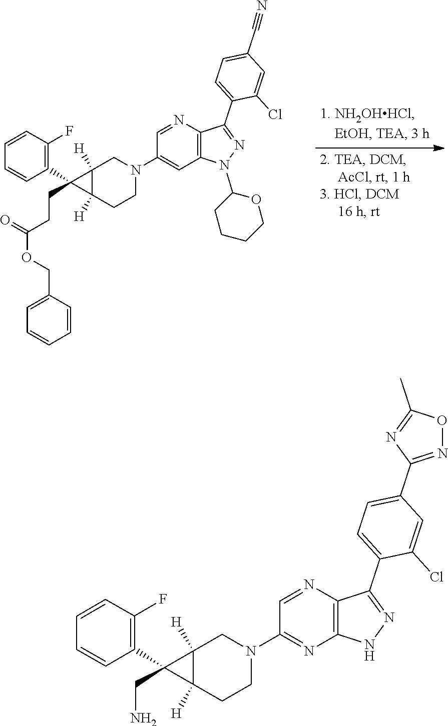 [SMILES: N#Cc1ccc(-c2nn(C3CCCCO3)c3cc(N4CC[C@@H]5[C@H](C4)[C@@]5(CCC(=O)OCc4ccccc4)c4ccccc4F)cnc23)c(Cl)c1 ; Cc1nc(-c2ccc(-c3n[nH]c4nc(N5CC[C@@H]6[C@H](C5)[C@@]6(CN)c5ccccc5F)cnc34)c(Cl)c2)no1]

Benzyl (((1S,6R,7R)-3-(3-(2-chloro-4-cyanophenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as described for Example 2 using Intermediate 44, 51 and 3-chloranyl-4-(4,4,5,5-tetramethyl-1,3,2-dioxaborolan-2-yl)benzenecarbonitrile) (27 mg, 0.040 mmol) was dissolved in EtOH then NH₂OH·HCl (4.6 mg, 0.07 mmol) and TEA (9.22 uL, 0.070 mmol) were sequentially added. The mixture was stirred for 3 h at 80° C. then concentrated in vacuo. EtOAc (35 mL) was added, and the resulting solution was washed with brine, dried over Na₂SO₄, filtered and concentrated. The obtained crude (27 mg, 0.04 mmol) was directly used in the next step without further purification (LCMS (ES⁺) Method 2: m/z 726 (M+H)⁺, RT 1.93 min). A solution benzyl (((1S,6R,7R)-3-(3-(2-chloro-4-(N-hydroxycarbamimidoyl)phenyl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (27 mg, 0.04 mmol) was treated with a solution of TEA (0.1 mL, 0.070 mmol) in DCM (1 mL) and AcCl (0.004 mL, 0.050 mmol). The resulting mixture was stirred for 1 h at rt and diluted with EtOAc (35 mL), washed with brine (2×5 mL), dried over Na₂SO₄, filtered, and concentrated in vacuo. The crude material was dissolved in toluene (1 mL) and stirred at 115° C. for 2 h. The mixture was concentrated in vacuo and the crude material was purified by flash chromatography (0-100% EtOAc in petroleum ether) to afford the title compound as a yellow solid (8 mg, 26% over two steps; LCMS (ES⁺) Method 2: m/z 749 (M+H)⁺, RT 2.6 min). This residue was dissolved in HCl (37%, 0.6 mL, 0.030 mmol) and stirred at rt for 16 h. After concentration in vacuo the residue was purified by preparative HPLC to obtain the title compound as a white solid (TFA salt; 1 mg, 17%). ¹H NMR (400 MHz, DMSO-d₆) δ 13.53 (s, 1H), 8.47 (s, 1H), 8.16 (br s, 1H), 8.12-8.04 (m, 2H), 7.66 (bs s, 3H), 7.51-7.47 (m, 1H), 7.40-7.35 (m, 1H), 7.25-7.19 (m, 2H), 4.48-4.44 (m, 1H), 4.37-4.34 (m, 1H), 3.77-3.71 (m, 1H), 3.16-3.10 (m, 2H), 2.99-2.94 (m, 1H), 2.72 (s, 3H), 2.36-2.31 (m, 1H), 1.83-1.79 (m, 2H), 1.57-1.54 (m, 1H). LCMS (ES⁺) Method 2: m/z 531 (M+H)⁺, RT 1.4 min.

### Example 378: 2-((1S,6R,7R)-7-(aminomethyl)-7-(2-fluorophenyl)-3-azabicyclo[4.1.0]heptan-3-yl)-5-(4-chloro-2-methyl-2H-indazol-5-yl)-3-methyl-3,7-dihydro-4H-pyrrolo[2,3-d]pyrimidin-4-one (Trifluoro Acetate Salt)

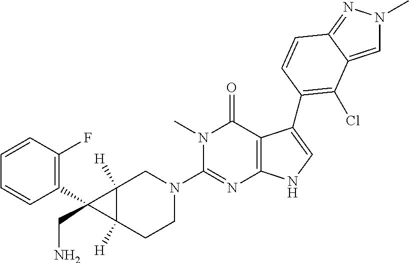 [SMILES: Cn1cc2c(Cl)c(-c3c[nH]c4nc(N5CC[C@@H]6[C@H](C5)[C@@]6(CN)c5ccccc5F)n(C)c(=O)c34)ccc2n1]

The above example was synthesized using the procedure reported for Example 62 using Intermediates 96, 51 and Intermediate 45 (DMSO as solvent was used in step 1 and HCl 37% followed by 2M NaOH was used in step 3); Example 378 was obtained as white solid (TFA salt; 11.7 mg, 50%). ¹H NMR (400 MHz, DMSO-d₆) δ 11.87 (s, 1H), 8.46 (s, 1H), 7.78 (br s, 3H), 7.56-7.53 (m, 2H), 7.46-7.41 (m, 1H), 7.37 (d, J=8.8 Hz, 1H), 7.30-7.24 (m, 2H), 7.11 (d, J=2.2 Hz, 1H), 4.25 (s, 3H), 3.79 (d, J=13.1 Hz, 1H), 3.67 (dd, J=13.4, 5.5 Hz, 1H), 3.60-3.57 (m, 2H), 3.42 (s, 3H), 3.38-3.36 (m, 1H), 2.36-2.30 (m, 2H), 2.11-2.01 (m, 1H), 1.76-1.71 (m, 1H), 1.66-1.62 (m, 1H). LCMS (ES⁺) Method 2: m/z 532 (M+H)⁺, RT 1.18 min.

### Example 379: 3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-thiadiazol-5-ol (Trifluoro Acetate Salt)

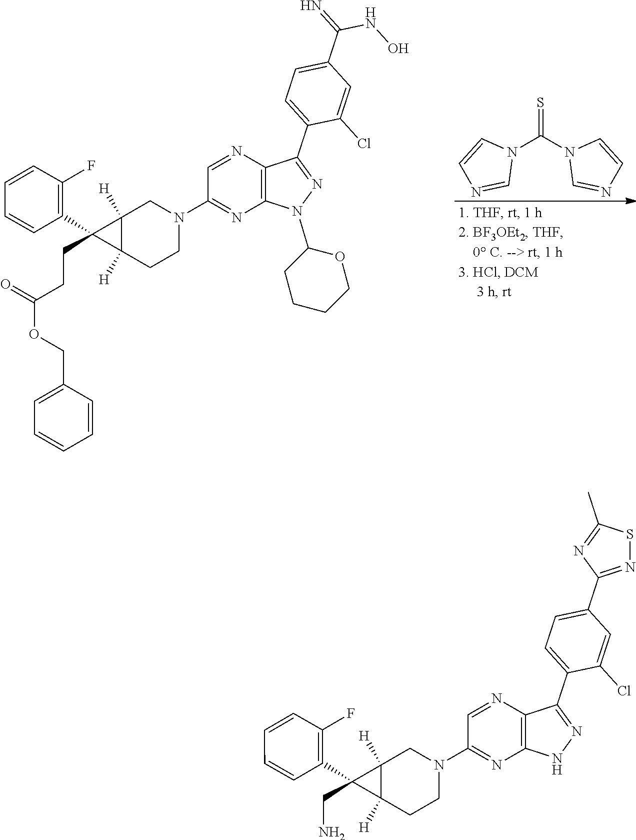 [SMILES: N=C(NO)c1ccc(-c2nn(C3CCCCO3)c3nc(N4CC[C@@H]5[C@H](C4)[C@@]5(CCC(=O)OCc4ccccc4)c4ccccc4F)cnc23)c(Cl)c1 ; S=C(n1ccnc1)n1ccnc1 ; Cc1nc(-c2ccc(-c3n[nH]c4nc(N5CC[C@@H]6[C@H](C5)[C@@]6(CN)c5ccccc5F)cnc34)c(Cl)c2)ns1]

TCDI (5.75 mg, 0.03 mmol) and (phenylmethyl)N-[[(1R,6S,7R)-4-[3-[2-chloranyl-4-(N-oxidanylcarbamimidoyl)phenyl]-1-(oxan-2-yl)pyrazolo[3,4-b]pyrazin-6-yl]-7-(2-fluorophenyl)-4-azabicyclo[4.1.0]heptan-7-yl]methyl]carbamate (prepared as described for Example 377 step 1; 19.5 mg, 0.03 mmol) were dissolved in THF (0.2 mL) and the obtained mixture was stirred at rt for 1 h. After completion, H₂O and EtOAc were added and the organic phase was isolated and washed with brine, dried over Na₂SO₄, filtered, and concentrated in vacuo. The crude was dissolved in THF (0.2 mL) and the mixture was treated at 0° C. with boron trifluoride etherate (0.02 mL, 0.13 mmol) and stirred at rt for 1 h. The reaction mixture was diluted with H₂O, extracted with EtOAc and the organic phase was washed with brine, dried over Na₂SO₄, filtered, and concentrated in vacuo. The crude material was purified by flash chromatography (0-100% EtOAc in petroleum ether then 0-50% MeOH in EtOAc) to afford the title compound as a colourless oil (LCMS (ES⁺) Method 2: m/z 767, 769 (M+H)⁺, RT 2.17 min) which was dissolved in HCl (37%, 0.5 mL) and stirred at rt for 3 h. After concentration in vacuo, the residue was purified by preparative HPLC to obtain the title compound as a white solid (TFA salt; 0.9 mg, 5.7%). LCMS (ES⁺) Method 2: m/z 549-551 (M+H)⁺, RT 1.24 min.

### Example 380: 6-(2-((1S,6R,7R)-7-(aminomethyl)-7-(2-fluorophenyl)-3-azabicyclo[4.1.0]heptan-3-yl)-3-methyl-4-oxo-4,7-dihydro-3H-pyrrolo[2,3-d]pyrimidin-5-yl)-5-fluoroisoquinolin-1(2H)-one (Trifluoro Acetate Salt)

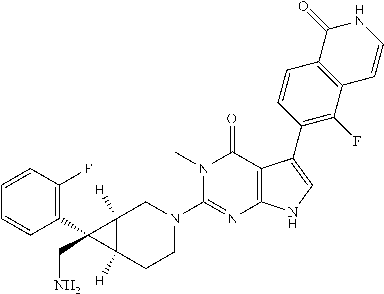 [SMILES: Cn1c(N2CC[C@@H]3[C@H](C2)[C@@]3(CN)c2ccccc2F)nc2[nH]cc(-c3ccc4c(=O)[nH]ccc4c3F)c2c1=O]

The above example was synthesized using the procedure reported for Example 62 using Intermediates 96, 51 and 5-fluoranyl-6-(4,4,5,5-tetramethyl-1,3,2-dioxaborolan-2-yl)-2H-isoquinolin-1-one (prepared as described in the synthesis of Intermediate 45 step 3; the reaction was carried out at 100° C.), HCl followed by treatment with NH₃ was used in the final deprotection step; Example 380 was obtained as white solid (TFA salt; 5 mg, 23%). LCMS (ES⁺) Method 2: m/z 529 (M+H)⁺, RT 1.1 min.

### Example 382: ((1S,6R,7R)-7-(2-fluorophenyl)-3-(5-(quinolin-4-yl)-5H-pyrrolo[2,3-b]pyrazin-2-yl)-3-azabicyclo[4.1.0]heptan-7-yl)methanamine (Trifluoro Acetate Salt)

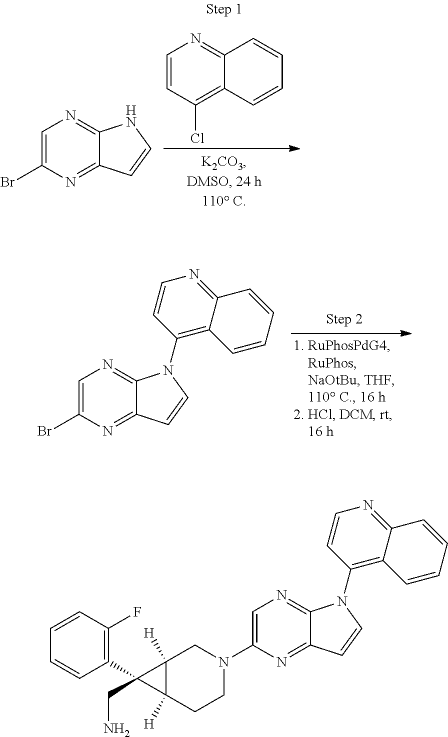

### Step 1: 4-(2-bromo-5H-pyrrolo[2,3-b]pyrazin-5-yl)quinoline

K₂CO₃ (418 mg, 3.03 mmol), 2-bromo-5H-pyrrolo[2,3-b]pyrazine (500 mg, 2.52 mmol) and 4-chloroquinoline (454 mg, 2.78 mmol) were dissolved in DMSO (10 mL) and stirred for 24 h at 110° C. After completion, the reaction mixture was diluted with EtOAc and washed with H₂O, brine, dried over Na₂SO₄, filtered, and concentrated in vacuo. The crude material was purified by flash chromatography (0-50% EtOAc in petroleum ether) to afford the title compound as white solid (230 mg, 28%). LCMS (ES⁺) Method 2: m/z 325, 327 (M+H)⁺, RT 1.26 min.

### Step 2: benzyl (((1S,6R,7R)-7-(2-fluorophenyl)-3-(5-(quinolin-4-yl)-5H-pyrrolo[2,3-b]pyrazin-2-yl)-3-azabicyclo[4.1.0]heptan-7-yl)methyl)carbamate

4-(2-bromo-5H-pyrrolo[2,3-b]pyrazin-5-yl)quinoline (20 mg, 0.06 mmol), (phenylmethyl)N-[[(1R,6S,7R)-7-(2-fluoranyl-3-methyl-phenyl)-4-azabicyclo[4.1.0]heptan-7-yl]methyl]carbamate hydrochloride (29 mg, 0.07 mmol), RuPhos (2.8 mg, 0.010 mmol), RuPhos Pd G4 (5.2 mg, 0.010 mmol) and NaOtBu (13 mg, 0.140 mmol) were sequentially dissolved in THF (0.4 mL) and the reaction was stirred for 16 h at 110° C. After completion, EtOAc (35 mL) was added and the obtained mixture was washed with brine (2×5 mL), dried over Na₂SO₄, filtered, and concentrated in vacuo. The crude material was purified by flash chromatography (0-50% EtOAc in petroleum ether) to afford the title compound as yellow solid (14 mg, 0.02 mmol) which was dissolved in EtOH (3 mL) and flowed for 1 h through the H-Cube using a Pd/C cartridge at 25° C. (5 bar H₂ pressure 1 mL/min flow rate). After concentration in vacuo, the residue was purified by preparative HPLC to obtain the title compound as yellow powder (TFA salt; 2.4 mg, 22%). LCMS (ES⁺) Method 2: m/z 465 (M+H)⁺, RT 1.24 min.

### Example 383: N-(((1S,6R,7R)-7-(2-fluorophenyl)-3-(3-(2-methyl-2H-indazol-5-yl)-1H-pyrazolo[3,4-b]pyrazin-6-yl)-3-azabicyclo[4.1.0]heptan-7-yl)methyl)acetamide (Trifluoro Acetate Salt)

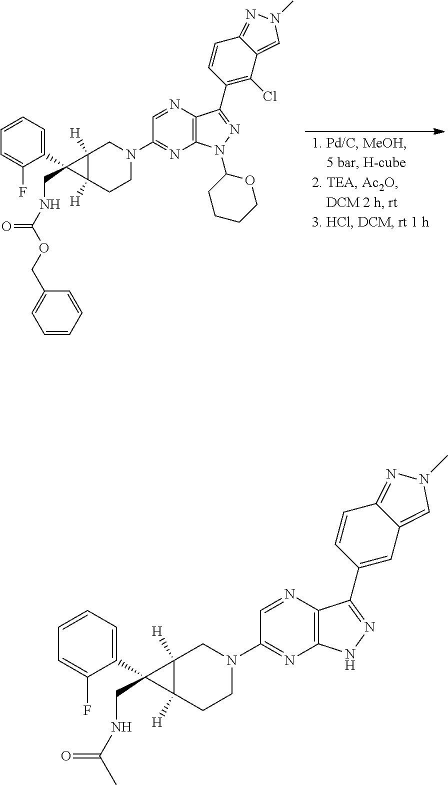 [SMILES: Cn1cc2c(Cl)c(-c3nn(C4CCCCO4)c4nc(N5CC[C@@H]6[C@H](C5)[C@@]6(CNC(=O)OCc5ccccc5)c5ccccc5F)cnc34)ccc2n1 ; CC(=O)NC[C@]1(c2ccccc2F)[C@@H]2CCN(c3cnc4c(-c5ccc6nn(C)cc6c5)n[nH]c4n3)C[C@@H]21]

Benzyl (((1S,6R,7R)-3-(3-(4-chloro-2-methyl-2H-indazol-5-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as described for Example 2 using Intermediates 51, 44 and 45; 23 mg, 0.013 mmol) was dissolved in MeOH (10 mL) and flowed for 20 min through the H-Cube using a Pd/C cartridge at 25° C. (5 bar H₂ pressure, and a 1 mL/min flow rate). Volatiles were concentrated in vacuo and the crude residue (5 mg, 0.01 mmol; LCMS (ES⁺) Method 2: m/z 553 (M+H)⁺, RT 1.51 min) was directly dissolved in DCM (1 mL), treated with TEA (2.5 uL, 0.02 mmol) and Ac₂O (1.7 uL, 0.02 mmol). After completion, the mixture was concentrated in vacuo and the residue was dissolved in HCl 4M in dioxane (45 uL, 0.18 mmol) and stirred at rt for 1 h. After removal of volatiles, the crude was purified by preparative HPLC to obtain the title compound as yellow powder (TFA salt; 0.6 mg, 13%). LCMS (ES⁺) Method 2: m/z 511 (M+H)⁺, RT 1.57 min.

### Example 384: 3-((2-amino-3-chloropyridin-4-yl)thio)-6-((1S,6R,7R)-7-(aminomethyl)-7-(2-fluorophenyl)-3-azabicyclo[4.1.0]heptan-3-yl)pyrazin-2-amine (Trifluoro Acetate Salt)

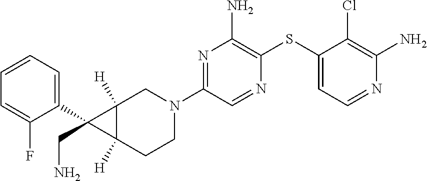 [SMILES: NC[C@]1(c2ccccc2F)[C@@H]2CCN(c3cnc(Sc4ccnc(N)c4Cl)c(N)n3)C[C@@H]21]

The above example was synthesized using the procedure reported for Example 71 using Intermediates 42 and 51 (the reaction was carried out at 100° C.); Example 384 was obtained as yellow solid (TFA salt; 11 mg, 94%). LCMS (ES⁺) Method 2: m/z 472 (M+H)⁺, RT 1.0 min.

### Example 385: 4-((6-((1S,6R,7R)-7-(aminomethyl)-7-(2-fluorophenyl)-3-azabicyclo[4.1.0]heptan-3-yl)pyrido[2,3-b]pyrazin-2-yl)thio)-3-chloropyridin-2-amine (Trifluoro Acetate Salt)

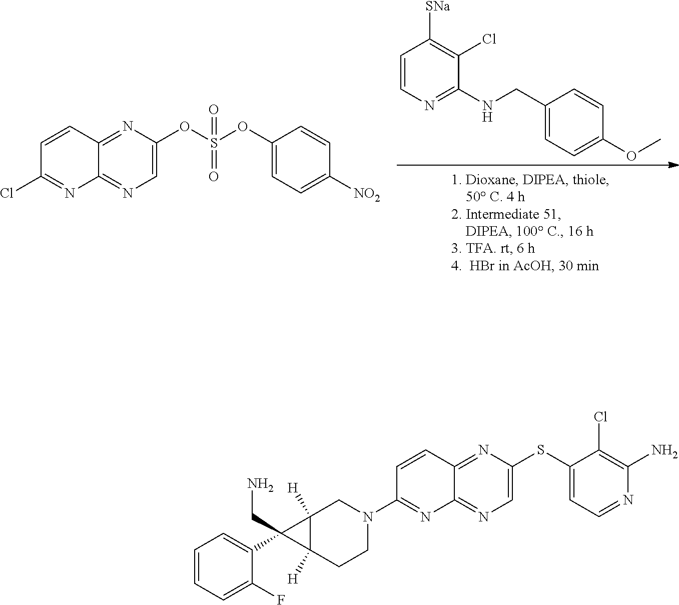

Intermediate 97 (25 mg, 0.07 mmol) and Intermediate 98 (21 mg, 0.07 mmol) in 1,4-dioxane (1.4 mL) was treated with DIPEA (0.03 mL, 0.15 mmol) and heated at 60° C. for 4 h. After completion, Intermediate 51 (27 mg, 0.07 mmol) and DIPEA (0.03, 0.15 mmol) were added, and the mixture was stirred for 16 h at 100° C. After concentration in vacuo, the reaction crude was purified by flash chromatography (0-100% MeCN in H₂O+0.1% TFA) to afford the title compound as a brown solid (13 mg, 0.017 mmol, 25%; LCMS (ES⁺) Method 2: m/z 762, 764 (M+H)⁺, RT 2.65 min) which was dissolved in TFA (1 mL) and stirred for 1 h at rt, and then concentrated in vacuo. The crude was purified by preparative HPLC to obtain the title compound as brown solid (TFA salt; 1 mg, 9%). LCMS (ES⁺) Method 2: m/z 642, 644 (M+H)⁺, RT 1.95 min.

This solid was dissolved in HBr in AcOH (33%, 0.5 mL) and stirred at rt for 1 h. After volatiles removal in vacuo the reaction crude was purified by preparative HPLC to obtain the title compound as yellow solid (TFA salt; 0.7 mg, 90%). LCMS (ES⁺) Method 2: m/z 508 (M+H)⁺, RT 1.07 min.

### Example 386: ((S)-1-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-yl)pyrrolidin-3-yl)methanol (Trifluoro Acetate Salt)

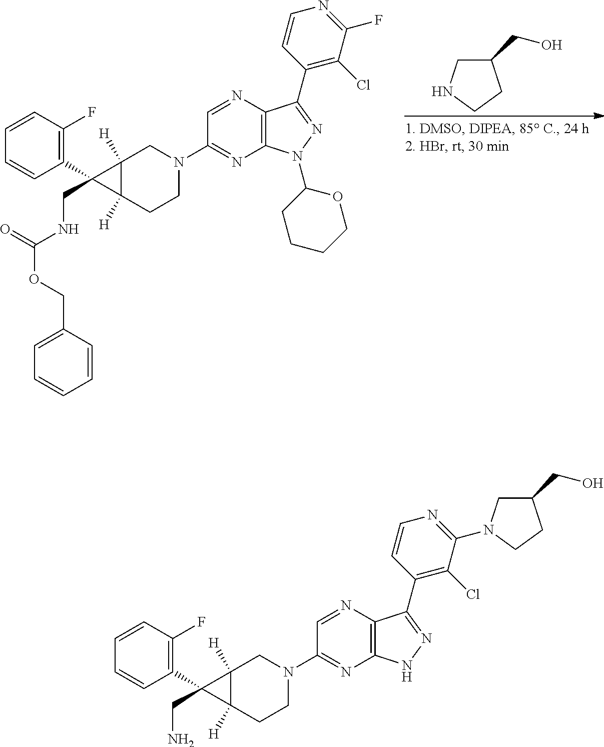 [SMILES: O=C(NC[C@]1(c2ccccc2F)[C@@H]2CCN(c3cnc4c(-c5ccnc(F)c5Cl)nn(C5CCCCO5)c4n3)C[C@@H]21)OCc1ccccc1 ; OC[C@H]1CCNC1 ; NC[C@]1(c2ccccc2F)[C@@H]2CCN(c3cnc4c(-c5ccnc(N6CC[C@H](CO)C6)c5Cl)n[nH]c4n3)C[C@@H]21]

Benzyl (((1S,6R,7R)-3-(3-(3-chloro-2-fluoropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (prepared as described for Example 2 using Intermediate 44, 51 and (3-chloro-2-fluoropyridin-4-yl)boronic acid) (21 mg, 0.03 mmol) was dissolved in DMSO (1.2 mL) and (S)-pyrrolidin-3-ylmethanol hydrochloride (16.8 mg, 0.12 mmol) and DIPEA (16 μL, 0.09 mmol) were sequentially added. The reaction was stirred at 85° C. for 24 h then poured in H₂O and extracted with EtOAc (3×10 mL). The combined organic layers were washed with brine, dried over Na₂SO₄, filtered, and concentrated in vacuo. The crude material was purified flash chromatography (0-100% EtOAc in petroleum ether) to afford the title compound as colourless oil (11 mg, 0.01 mmol, 47%; LCMS (ES⁺) Method 2: m/z 767 (M+H)⁺, RT 2.22 min), which was subsequentially treated with HBr 33% in AcOH (HBr 33%, 0.7 mL) and stirred at rt for 30 min. After volatiles removal in vacuo, the residue was purified by preparative HPLC to afford the title compound as yellow solid (TFA salt; 7 mg, 89%). LCMS (ES⁺) Method 2: m/z 549 (M+H)⁺, RT 1.02 min.

### Example 387: 4-((6-((1R,6S,7S)-7-(aminomethyl)-7-(2-fluorophenyl)-3-azabicyclo[4.1.0]heptan-3-yl)-1,5-naphthyridin-2-yl)thio)-3-chloropyridin-2-amine (Trifluoro Acetate Salt)

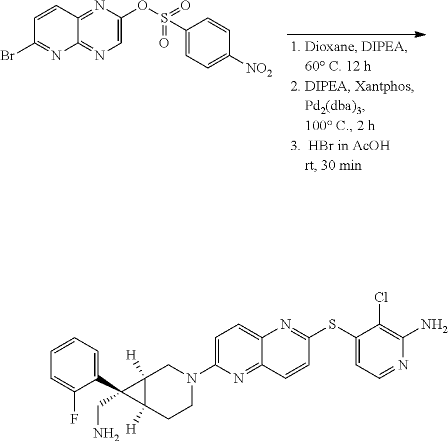 [SMILES: O=[N+]([O-])c1ccc(S(=O)(=O)Oc2cnc3nc(Br)ccc3n2)cc1 ; NC[C@@]1(c2ccccc2F)[C@@H]2CCN(c3ccc4nc(Sc5ccnc(N)c5Cl)ccc4n3)C[C@@H]21]

Intermediate 51 (50 mg, 0.13 mmol) was dissolved in 1,4-dioxane (0.54 mL) and the mixture was cooled to 0° C. then treated with Intermediate 99 (35 mg, 0.09 mmol). The reaction was heated at 60° C. for 16 h then the solvent was concentrated in vacuo. The crude product was purified by flash chromatography (0-20% MeOH in DCM) to afford the title compound as a yellow sticky solid (26 mg, 54%; LCMS (ES⁺) Method 2: m/z 561, 563 (M+H)⁺, RT 2.33 min). This residue (13 mg, 0.02 mmol), (2-azanyl-3-chloranyl-pyridin-4-yl)sulfanylsodium (6.33 mg, 0.03 mmol), Pd₂(dba)₃ (2.1 mg, 0.002 mmol), XantPhos (2.68 mg, 0.005 mmol) were dissolved in 1,4-dioxane (0.5 mL) then DIPEA (12 μL, 0.07 mmol) was added and the mixture was heated at 100° C. for 2 h. The reaction mixture was filtered on a pad of SiO₂ washing with DCM and then concentrated in vacuo to afford the title compound, which was used without further purification (10 mg, 0.016 mmol, 67%). LCMS (ES⁺) Method 2: m/z 641, 643 (M+H)⁺, RT 2.01 min. This crude was dissolved in HBr in AcOH (33%; 0.7 mL) and stirred at rt for 30 min. After concentration in vacuo, the residue was purified by preparative HPLC to obtain the title compound as yellow solid (TFA salt; 3.6 mg, 45%). LCMS (ES⁺) Method 2: m/z 507 (M+H)⁺, RT 1.16 min.

### Example 388: ((1S,6R,7R)-3-(5-((3-chloro-2-(1H-pyrazol-1-yl)pyridin-4-yl)thio)pyrazin-2-yl)-7-(2-fluorophenyl)-3-azabicyclo[4.1.0]heptan-7-yl)methanamine (Trifluoro Acetate Salt)

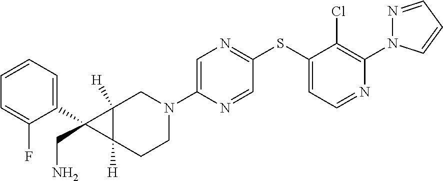 [SMILES: NC[C@]1(c2ccccc2F)[C@@H]2CCN(c3cnc(Sc4ccnc(-n5cccn5)c4Cl)cn3)C[C@@H]21]

The above example was synthesized using the procedure reported for Example 71 using Intermediates 101 and 51 (the reaction was carried out at 100° C. in 1,4-dioxane); Example 388 was obtained as pale-yellow solid (TFA salt, 9 mg, 28%). LCMS (ES⁺) Method 2: m/z 508, (M+H)⁺, RT 1.32 min.

### Example 394: ((1R,6S,7S)-7-(2-fluorophenyl)-3-(6-(2-methyl-2H-indazol-5-yl)-1,5-naphthyridin-2-yl)-3-azabicyclo[4.1.0]heptan-7-yl)methanamine (Trifluoro Acetate Salt)

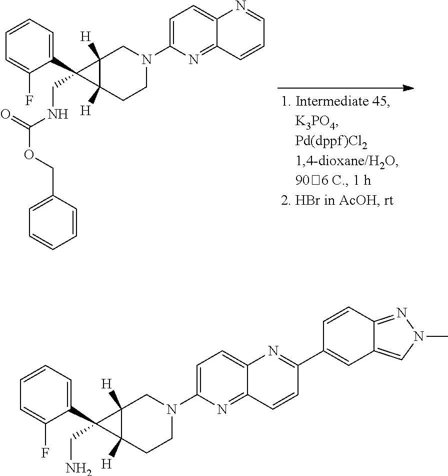 [SMILES: O=C(NC[C@]1(c2ccccc2F)[C@@H]2CN(c3ccc4ncccc4n3)CC[C@@H]21)OCc1ccccc1 ; Cn1cc2cc(-c3ccc4nc(N5CC[C@H]6[C@@H](C5)[C@]6(CN)c5ccccc5F)ccc4n3)ccc2n1]

Benzyl (((1R,6S,7S)-3-(6-bromo-1,5-naphthyridin-2-yl)-7-(2-fluorophenyl)-3-azabicyclo[4.1.0]heptan-7-yl)methyl)carbamate (prepared as described in the synthesis of Example 387) (13 mg, 0.023 mmol) and Intermediate 45 were reacted according to the procedure reported for the synthesis of Example 2, step 2 and 3 (using HBr (33% in AcOH) in the deprotection step). The title compound was obtained as yellow powder (TFA salt, 1.5 mg, 13%). LCMS (ES⁺) Method 2: m/z 479 (M+H)⁺, RT 1.09 min.

### Example 395: 4-((2-((1S,6R,7R)-7-(aminomethyl)-7-(2-fluorophenyl)-3-azabicyclo[4.1.0]heptan-3-yl)pyrido[2,3-b]pyrazin-6-yl)thio)-3-chloropyridin-2-amine (Trifluoro Acetate Salt)

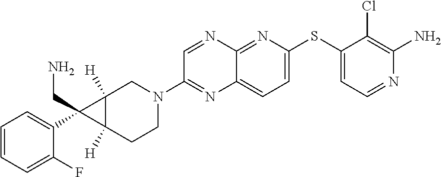 [SMILES: NC[C@]1(c2ccccc2F)[C@@H]2CCN(c3cnc4nc(Sc5ccnc(N)c5Cl)ccc4n3)C[C@@H]21]

Intermediate 97 (10 mg, 0.027 mmol) and Intermediate 51 (27 mg, 0.068 mmol) in 1,4-dioxane (0.5 mL) were treated with DIPEA (0.02 mL, 0.14 mmol) and the mixture was heated at 60° C. for 16 h. After completion, Intermediate 98 (16 mg, 0.05 mmol) and DIPEA (100 μL, 0.7 mmol) were sequentially added and the mixture was stirred at 80° C. for 16 h. The solvent was concentrated in vacuo and the crude material purified by flash chromatography (C18; 0-100% MeCN/H₂O+0.1% TFA) to afford the title compound as brown solid (39 mg; LCMS (ES⁺) Method 2: m/z 762, 763 (M+H)⁺, RT 2.5 min). The residue was subjected at the procedure reported for Example 385, step 2 and obtained as yellow solid (TFA salt; 7 mg, 40% over two steps). LCMS (ES⁺) Method 2: m/z 642, 644 (M+H)⁺, RT 1.97 min. The final deprotection step was the same reported in Example 385, step 3. Example 395 was obtained as yellow solid (TFA salt, 1.9 mg, 13%). ¹H NMR (400 MHz, DMSO-d₆) δ 8.98 (s, 1H), 8.05 (d, J=8.7 Hz, 1H), 7.82 (d, J=5.2 Hz, 1H), 7.66, 7.64 (br s, 3H), 7.51-7.47 (m, 1H), 7.40-7.36 (s, 1H), 7.52-7.21 (m, 2H), 6.62-6.58 (m, 1H), 6.47 (d, J=5.7 Hz, 1H), 4.56 (d, J=15.3 Hz, 1H), 4.50-4.44 (m, 1H), 3.80 (dd, J=15.3, 7.9 Hz, 1H), 3.11-3.06 (m, 2H), 3.01-2.92 (m, 1H), 2.36-2.33 (m, 1H), 1.83-1.79 (m, 2H), 1.59-1.56 (m, 1H). LCMS (ES⁺) Method 2: m/z 508 (M+H)⁺, RT 1.14 min.

### Example 396: ((1S,6R,7R)-3-(3-(7-chloro-1-methyl-1H-benzo[d]imidazol-6-yl)-1H-pyrazolo[3,4-b]pyrazin-6-yl)-7-(2,5-difluorophenyl)-3-azabicyclo[4.1.0]heptan-7-yl)methanamine (trifluoro acetate salt)

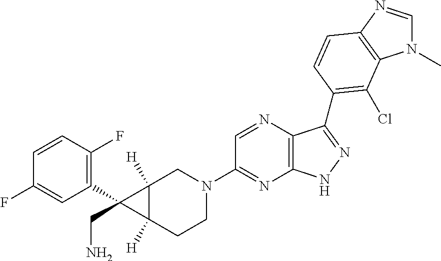 [SMILES: Cn1cnc2ccc(-c3n[nH]c4nc(N5CC[C@@H]6[C@H](C5)[C@@]6(CN)c5cc(F)ccc5F)cnc34)c(Cl)c21]

The above example was synthesized using the procedure reported for Example 2 using Intermediates 102, 44, and 7-chloro-1-methyl-6-(4,4,5,5-tetramethyl-1,3,2-dioxaborolan-2-yl)-1H-benzo[d]imidazole (prepared as reported for the synthesis of Intermediate 15); HBr (33% in AcOH) was used in the final deprotection step; Example 396 was obtained as yellow solid (TFA salt; 4 mg, 22%). ¹H NMR (400 MHz, DMSO-d₆) δ 13.36 (s, 1H), 8.41 (s, 1H), 8.37 (s, 1H), 7.73 (d, J=8.3 Hz, 1H), 7.68 (br s, 3H), 7.51 (d, J=8.3 Hz, 1H), 7.34-7.22 (m, 3H), 4.45 (d, J=14.9 Hz, 1H), 4.36-4.32 (m, 1H), 4.17 (s, 3H), 3.70 (dd, J=14.0, 5.7 Hz, 1H), 3.18-3.03 (m, 2H), 2.97-2.89 (m, 1H), 2.37-2.30 (m, 1H), 1.88-1.78 (m, 2H), 1.61-1.57 (m, 1H). LCMS (ES⁺) Method 2: m/z 521 (M+H)⁺, RT 1.1 min.

### Example 397: 4-((6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)thio)-3-chloropyridin-2-amine (Trifluoro Acetate Salt)

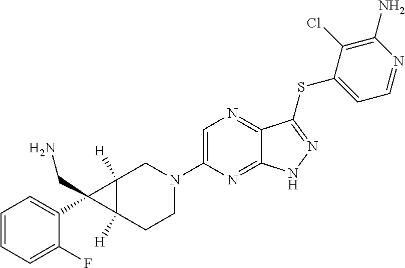 [SMILES: NC[C@]1(c2ccccc2F)[C@@H]2CCN(c3cnc4c(Sc5ccnc(N)c5Cl)n[nH]c4n3)C[C@@H]21]

The above example was synthesized using the procedure reported for Example 53 step 1 using benzyl (((1S,6R,7R)-7-(2-fluorophenyl)-3-(3-iodo-1-(tetrahydro-2H-pyran-2-yl)-1H-pyrazolo[3,4-b]pyrazin-6-yl)-3-azabicyclo[4.1.0]heptan-7-yl)methyl)carbamate (prepared as reported in the synthesis of Example 2 in step 1 from Intermediates 44 and 51) and sodium 2-amino-3-chloropyridine-4-thiolate (prepared as reported in the synthesis of Intermediate 42 step 3); Example 397 was obtained as white solid (TFA salt, 4.0 mg, 18%). ¹H NMR (400 MHz, DMSO-d₆) δ 13.89 (s, 1H), 8.45 (s, 1H), 7.64 (br s, 3H), 7.57 (d, J=5.7 Hz, 1H), 7.50-7.46 (m, 1H), 7.41-7.36 (m, 1H), 7.25-7.19 (m, 2H), 6.51 (br s, 2H), 5.75 (d, J=5.5 Hz, 1H), 4.39 (br d, J=13.8 Hz, 1H), 4.32 (br d, J=9.8 Hz, 1H), 3.77-3.72 (m, 1H), 3.17-3.01 (m, 2H), 2.98-2.85 (m, 1H), 2.43-2.39 (m, 1H), 1.85-1.76 (m, 2H), 1.58-1.53 (m, 1H); LCMS (ES⁺) Method 2: m/z 497 (M+H)⁺, RT min 0.95.

### Example 398: ((1S,6R,7R)-3-(6-((3-chloro-2-(1H-pyrazol-1-yl)pyridin-4-yl)thio)pyrido[2,3-b]pyrazin-2-yl)-7-(2-fluorophenyl)-3-azabicyclo[4.1.0]heptan-7-yl)methanamine (trifluoro acetate salt)

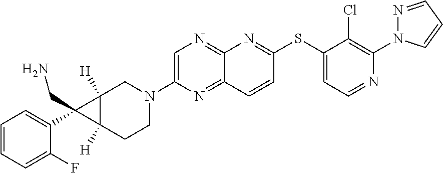 [SMILES: NC[C@]1(c2ccccc2F)[C@@H]2CCN(c3cnc4nc(Sc5ccnc(-n6cccn6)c5Cl)ccc4n3)C[C@@H]21]

The above example was synthesized using the procedure reported for Example 395 using Intermediates 97, 51 and 100 (step 3); Example 398 was obtained as white solid (TFA salt, 11 mg, 27%). ¹H NMR (400 MHz, DMSO-d₆) δ 8.37 (d, J=5.3 Hz, 1H), 8.29 (d, J=2.4 Hz, 1H), 8.13 (d, J=8.6 Hz, 1H), 7.87 (d, J=8.6 Hz, 1H), 7.83 (s, 1H), 7.65 (br s, 3H), 7.49 (br t, J=7.7 Hz, 1H), 7.29-7.14 (m, 2H), 6.63-6.52 (m, 1H), 4.56 (br d, J=14.3 Hz, 1H), 4.46 (br d, J=10.7 Hz, 2H), 3.84 (br dd, J=14.6, 6.5 Hz, 2H), 2.92-3.19 (m, 4H), 2.29-2.42 (m, 1H), 1.86-1.73 (m, 2H), 1.63-1.51 (m, 1H); LCMS (ES⁺) Method 2: m/z 560 (M+H)⁺, RT 1.22 min.

### Example 400: ((1S,6R,7R)-3-(6-((4-chloro-2-methyl-2H-indazol-5-yl)thio)pyrido[2,3-b]pyrazin-2-yl)-7-(2-fluorophenyl)-3-azabicyclo[4.1.0]heptan-7-yl)methanamine (Trifluoro Acetate Salt)

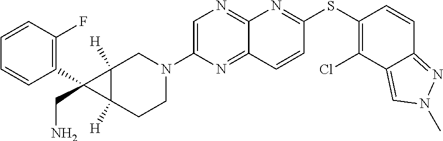 [SMILES: Cn1cc2c(Cl)c(Sc3ccc4nc(N5CC[C@@H]6[C@H](C5)[C@@]6(CN)c5ccccc5F)cnc4n3)ccc2n1]

The above example was synthesized using the procedure reported for Example 395 using Intermediates 97, 51 and 110. Example 400 was obtained as yellow solid (TFA salt; 0.6 mg, 3%). LCMS (ES⁺) Method 2: m/z 546 (M+H)⁺, RT 1.37 min.

### Example 401: 4-((5-((1S,6R,7R)-7-(aminomethyl)-7-(2-fluorophenyl)-3-azabicyclo[4.1.0]heptan-3-yl)pyrazin-2-yl)thio)-3-chloropyridin-2-amine (Trifluoro Acetate Salt)

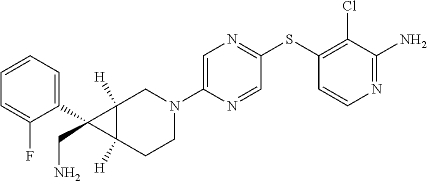 [SMILES: NC[C@]1(c2ccccc2F)[C@@H]2CCN(c3cnc(Sc4ccnc(N)c4Cl)cn3)C[C@@H]21]

The above compound was synthesized according to the procedure reported for Example 71 using 3-chloro-4-((5-chloropyrazin-2-yl)thio)pyridin-2-amine (prepared as reported in the synthesis of Intermediate 101, step 2-4) and Intermediate 51 in 1-BuOH at 100° C.; Example 401 was obtained as a white solid (TFA salt; 3.2 mg, 27%). LCMS (ES⁺) Method 2: m/z 457 (M+H)⁺, RT 1.02 min.

### Example 402: ((1S,6R,7R)-3-(5-((3-chloro-2-(1H-pyrazol-1-yl)pyridin-4-yl)thio)pyrazin-2-yl)-7-(2,5-difluorophenyl)-3-azabicyclo[4.1.0]heptan-7-yl)methanamine (Trifluoro Acetate Salt)

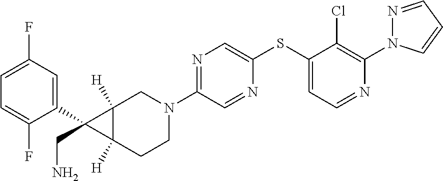 [SMILES: NC[C@]1(c2cc(F)ccc2F)[C@@H]2CCN(c3cnc(Sc4ccnc(-n5cccn5)c4Cl)cn3)C[C@@H]21]

The above example was synthesized using the procedure reported for Example 71 using Intermediates 101 and 102 (the reaction was carried out at 100° C. in 1-BuOH); Example 402 was obtained as white solid (TFA salt, 11 mg, 27%). LCMS (ES⁺) Method 2: m/z 526 (M+H)⁺, RT 1.34 min.

### Example 403: ((1R,7S)-4-(5-((3-chloro-2-(1H-pyrazol-1-yl)pyridin-4-yl)thio)pyrazin-2-yl)-8-(2-fluorophenyl)-4-azabicyclo[5.1.0]octan-8-yl)methanamine (Trifluoro Acetate Salt)

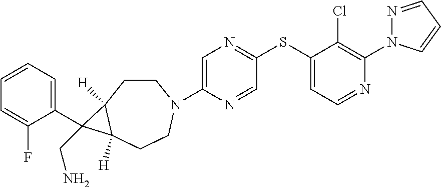 [SMILES: NCC1(c2ccccc2F)[C@@H]2CCN(c3cnc(Sc4ccnc(-n5cccn5)c4Cl)cn3)CC[C@@H]21]

The above example was synthesized using the procedure reported for Example 71 using Intermediates 101 and 103 (the reaction was carried out at 100° C. in 1,4-dioxane; HBr (33% in AcOH) was used in the deprotection step); Example 403 was obtained as pale-yellow solid (TFA salt, 8 mg, 29%). LCMS (ES⁺) Method 2: m/z 522 (M+H)⁺, RT 1.46 min.

### Example 404: ((1R,7S)-4-(3-(4-chloro-2-methyl-2H-indazol-5-yl)-1H-pyrazolo[3,4-b]pyrazin-6-yl)-8-(2-fluorophenyl)-4-azabicyclo[5.1.0]octan-8-yl)methanamine (Trifluoro Acetate Salt)

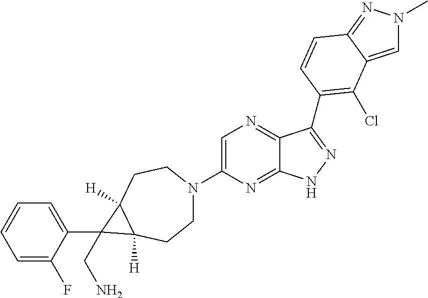 [SMILES: Cn1cc2c(Cl)c(-c3n[nH]c4nc(N5CC[C@@H]6[C@H](CC5)C6(CN)c5ccccc5F)cnc34)ccc2n1]

The above example was synthesized using the procedure reported for Example 2 using Intermediates 103, 44, and 45; HBr (33% in AcOH) was used in the final deprotection step; Example 404 was obtained as yellow solid (TFA salt; 6 mg, 56%). ¹H NMR (400 MHz, DMSO-d₆) δ 13.32 (s, 1H), 8.56 (s, 1H), 8.40 (s, 1H), 7.67 (br s, 2H), 7.59 (br s, 3H), 7.46-7.40 (m, 1H), 7.38-7.34 (m, 1H), 7.22-7.16 (m, 2H), 4.56-4.52 (m, 2H), 4.24 (s, 3H), 3.34-3.28 (m, 2H), 3.0 (br s, 2H), 2.50-2.45 (m, 2H), 1.53 (br s, 4H). LCMS (ES⁺) Method 2: m/z 517 (M+H)⁺, RT 1.27 min.

### Example 405: ((1S,6R,7R)-3-(3-(4-chloro-1H-indol-5-yl)-1H-pyrazolo[3,4-b]pyrazin-6-yl)-7-(2-fluorophenyl)-3-azabicyclo[4.1.0]heptan-7-yl)methanamine (Trifluoro Acetate Salt)

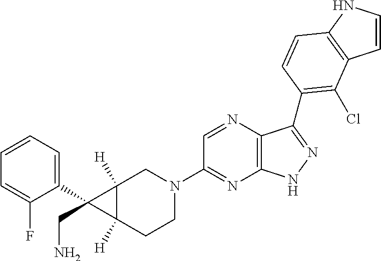 [SMILES: NC[C@]1(c2ccccc2F)[C@@H]2CCN(c3cnc4c(-c5ccc6[nH]ccc6c5Cl)n[nH]c4n3)C[C@@H]21]

The above example was synthesized using the procedure reported for Example 2 using tert-butyl (((1S,6R,7R)-7-(2-fluorophenyl)-3-azabicyclo[4.1.0]heptan-7-yl)methyl)carbamate (prepared as reported for the synthesis of Intermediate 9 using 2-(2-fluorophenyl)acetonitrile and benzyl (3aS,7aR)-tetrahydro-[1,3,2]dioxathiolo[4,5-c]pyridine-5(4H)-carboxylate 2,2-dioxide in step 1), Intermediate 44, and 4-chloro-5-(4,4,5,5-tetramethyl-1,3,2-dioxaborolan-2-yl)-1H-indole (prepared as described for Intermediate 15); TFA in DCM was used in the final deprotection step; Example 405 was obtained as a yellow solid (TFA salt; 1.9 mg, 17%). LCMS (ES⁺) Method 2: m/z 488 (M+H)⁺, RT 1.21 min.

### Example 406: ((1S,6R,7R)-3-(6-(3,4-dihydro-1,5-naphthyridin-1(2H)-yl)pyrido[2,3-b]pyrazin-2-yl)-7-(2-fluorophenyl)-3-azabicyclo[4.1.0]heptan-7-yl)methanamine (Trifluoro Acetate Salt)

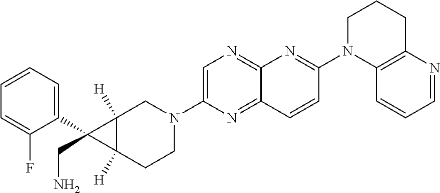 [SMILES: NC[C@]1(c2ccccc2F)[C@@H]2CCN(c3cnc4nc(N5CCCc6ncccc65)ccc4n3)C[C@@H]21]

Benzyl (((1 S,6R,7R)-3-(6-chloropyrido[2,3-b]pyrazin-2-yl)-7-(2-fluorophenyl)-3-azabicyclo[4.1.0]heptan-7-yl)methyl)carbamate (prepared as described in Example 400, using Intermediate 51, 97; 20 mg, 0.04 mmol), 1,2,3,4-tetrahydro-1,5-naphthyridine (5 mg, 0.04 mmol), XantPhos Pd G4 (1.8 mg, 0.02 mmol) and Cs₂CO₃ (25 mg, 0.077 mmol) were dissolved in toluene and the mixture was degassed and heated at 60° C. for 16 h. The solvent was concentrated, and the reaction crude was purified by flash chromatography (0-100% MeCN/H₂O+0.1% TFA) to afford the title compound as a yellow solid (12.7 mg, 24%; LCMS (ES⁺) Method 2: m/z 616 (M+H)⁺, RT 1.63 min). This solid was dissolved in HBr in AcOH (33%, 0.5 mL) and stirred at rt for 1 h. After volatiles removal in vacuo the reaction crude was purified by preparative HPLC to obtain the title compound as yellow solid (TFA salt; 3 mg, 16%). LCMS (ES⁺) Method 2: m/z 482 (M+H)⁺, RT 0.9 min.

### Example 407: ((1S,6R,7R)-7-(2-fluorophenyl)-3-(6-(pyridin-4-ylthio)pyrido[2,3-b]pyrazin-2-yl)-3-azabicyclo[4.1.0]heptan-7-yl)methanamine (Trifluoro Acetate Salt)

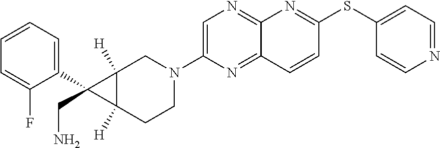 [SMILES: NC[C@]1(c2ccccc2F)[C@@H]2CCN(c3cnc4nc(Sc5ccncc5)ccc4n3)C[C@@H]21]

The above compound was synthesized according to the procedure reported for Example 400 using sodium pyridine-4-thiolate (prepared as reported for Intermediate 98 step 2 & 3 starting from 4-bromopyridine hydrochloride); Example 407 was obtained as yellow solid (TFA salt; 1 mg, 6%). LCMS (ES⁺) Method 2: m/z 459 (M+H)⁺, RT 0.98 min.

### Example 408: 2-((1S,6R,7R)-7-(aminomethyl)-7-(2-fluorophenyl)-3-azabicyclo[4.1.0]heptan-3-yl)-7-(2,3-dichlorophenyl)-4H-pyrido[1,2-a]pyrimidin-4-one (Trifluoro Acetate Salt)

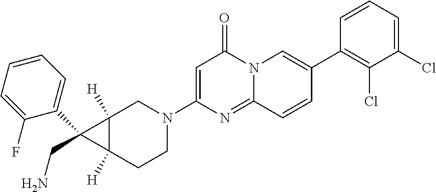 [SMILES: NC[C@]1(c2ccccc2F)[C@@H]2CCN(c3cc(=O)n4cc(-c5cccc(Cl)c5Cl)ccc4n3)C[C@@H]21]

The above example was synthesized using the procedure reported for Example 2 using 7-bromo-2-chloro-4H-pyrido[1,2-a]pyrimidin-4-one and Intermediate 51 in 1,4-dioxane in step 1; HBr (33% in AcOH) was used in the final deprotection step; Example 408 was obtained as a white powder (TFA salt; 1.1 mg, 7%). LCMS (ES⁺) Method 2: m/z 509 (M+H)⁺, RT 1.45 min.

### Example 409: ((1S,6R,7R)-3-(6-(4-fluorophenoxy)pyrido[2,3-b]pyrazin-2-yl)-7-(2-fluorophenyl)-3-azabicyclo[4.1.0]heptan-7-yl)methanamine (Trifluoro Acetate Salt)

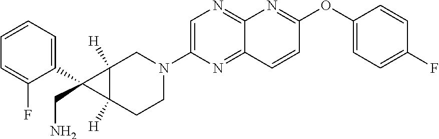 [SMILES: NC[C@]1(c2ccccc2F)[C@@H]2CCN(c3cnc4nc(Oc5ccc(F)cc5)ccc4n3)C[C@@H]21]

Benzyl (((1S,6R,7R)-3-(6-chloropyrido[2,3-b]pyrazin-2-yl)-7-(2-fluorophenyl)-3-azabicyclo[4.1.0]heptan-7-yl)methyl)carbamate (prepared as described in the Example 400; 25 mg, 0.077 mmol), 4-fluorophenol (8.6 mg, 0.077 mmol), Cs₂CO₃ (25 mg, 0.077 mmol) and DIPEA (27 μL, 0.15 mmol) were dissolved in 1,4-dioxane (0.5 mL). The mixture was stirred at 150° C. for 3 h under MW irradiation then filtered on a pad of silica eluting with a mixture of DCM:MeOH (9:1; 10 mL). Volatiles were concentrated in vacuo and the reaction crude was purified by preparative HPLC to afford the title compound as a yellow solid (TFA salt; 1.3 mg, 7%). LCMS (ES⁺) Method 2: m/z 460 (M+H)⁺, RT 1.39 min.

### Example 410: 7-((2-amino-3-chloropyridin-4-yl)thio)-2-((1S,6R,7R)-7-(aminomethyl)-7-(2-fluorophenyl)-3-azabicyclo[4.1.0]heptan-3-yl)-4H-pyrido[1,2-a]pyrimidin-4-one (trifluoro acetate salt)

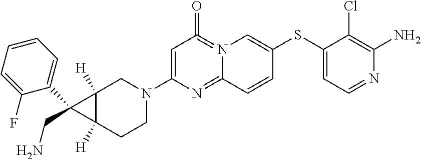

The above example was synthesized using the procedure reported for Example 387 using 7-bromo-2-chloro-4H-pyrido[1,2-a]pyrimidin-4-one and tert-butyl (((1S,6R,7R)-7-(2-fluorophenyl)-3-azabicyclo[4.1.0]heptan-7-yl)methyl)carbamate (prepared as reported in the synthesis of Intermediate 9 starting from benzyl (3aR,7aS)-tetrahydro-[1,3,2]dioxathiolo[4,5-c]pyridine-5(4H)-carboxylate 2,2-dioxide and 2-(2-fluorophenyl)acetonitrile) in step 1 and sodium 2-amino-3-chloropyridine-4-thiolate (prepared as reported in the synthesis of Intermediate 42 in step 3); TFA in DCM was used in the final deprotection step; Example 410 was obtained as white solid (TFA salt, 1.2 mg, 24%). LCMS (ES⁺) Method 2: m/z 523 (M+H)⁺, RT 1.09 min.

### Examples 411 & 412: 6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one (trifluoro acetate salt) & 6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one (Trifluoro Acetate Salt)

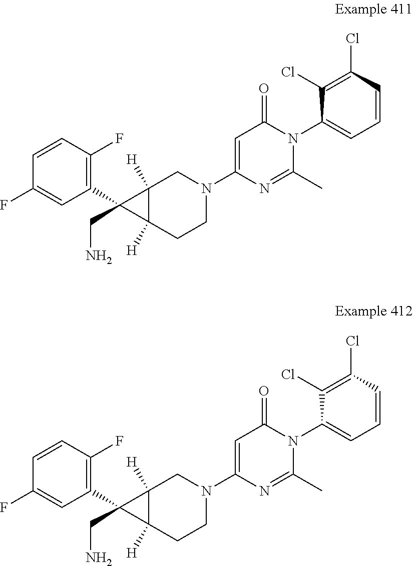

### Step 1: benzyl (((1S,6R,7R)-3-(1-(2,3-dichlorophenyl)-2-methyl-6-oxo-1,6-dihydropyrimidin-4-yl)-7-(2,5-difluorophenyl)-3-azabicyclo[4.1.0]heptan-7-yl)methyl)carbamate

A solution of Intermediate 111 (30.0 mg, 0.10 mmol) and Intermediate 102 (35.3 mg, 0.09 mmol) in DMF (0.5 mL) was treated with DIPEA (0.09 mL, 0.52 mmol) and stirred at 100° C. for 3 h. After cooling NH₄Cl sat. sol. was added and the mixture was extracted with EtOAc (3×). The combined organic layers were washed with NaHCO₃ sat. sol., brine and dried over Na₂SO₄, filtered, and concentrated in vacuo. The crude material was purified by flash chromatography (0-100% EtOAc in petroleum ether) to afford a pale-yellow solid as a mixture of atropoisomers (20 mg, 37%; LCMS (ES⁺) Method 2: m/z 625 (M+H)⁺, RT 2.30 min) which was dissolved in HBr (33% in AcOH; 0.5 mL) and stirred at rt for 20 min. After volatiles removal in vacuo, the residue was purified by preparative HPLC to obtain Example 411 as a white powder (0.7 mg, 15%; LCMS (ES⁺) Method 1: m/z 491 (M+H)⁺, RT 1.12 min) & Example 412 as a white powder (0.7 mg, 15%; LCMS (ES⁺) Method 1: m/z 491 (M+H)⁺, RT 1.19 min).

### Example 414: 3-((2-amino-3-chloropyridin-4-yl)thio)-6-((1S,6R,7R)-7-(aminomethyl)-7-(2,5-difluorophenyl)-3-azabicyclo[4.1.0]heptan-3-yl)pyrazin-2-amine (Trifluoro Acetate Salt)

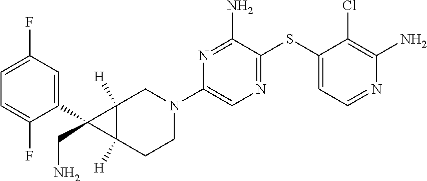 [SMILES: NC[C@]1(c2cc(F)ccc2F)[C@@H]2CCN(c3cnc(Sc4ccnc(N)c4Cl)c(N)n3)C[C@@H]21]

The above example was synthesized using the procedure reported for Example 384 using Intermediate 42 and 102 (in DMSO at 80° C.); Example 414 was obtained as a pale-yellow solid (TFA salt; 6.7 mg, 20%). ¹H NMR (600 MHz, DMSO-d₆) δ 7.72 (br s, 3H), 7.66 (d, J=5.7 Hz, 1H), 7.63 (s, 1H), 7.25-7.33 (m, 2H), 7.24-7.18 (m, 1H), 6.81 (br s, 2H), 6.26 (br s, 2H), 5.8 (br d, J=5.7 Hz, 1H), 4.26 (br d, J=13.8 Hz, 2H), 3.60 (br dd, J=14.1, 6.3 Hz, 3H), 3.19-3.09 (m, 1H), 3.09-3.00 (m, 1H), 2.76 (td, J=12.8, 3.5 Hz, 1H), 2.54 (s, 4H), 2.27 (br t, J=11.6 Hz, 1H), 1.82 (td, J=9.8, 4.11 Hz, 1H), 1.74-1.62 (m, 1H), 1.56-1.48 (m, 1H); LCMS (ES⁺) Method 2: m/z 490 (M+H)⁺, RT 0.94 min.

### Example 415: ((1S,6R,7R)-7-(2-fluorophenyl)-3-(6-(2-methyl-2H-indazol-5-yl)pyrido[2,3-b]pyrazin-2-yl)-3-azabicyclo[4.1.0]heptan-7-yl)methanamine (Trifluoro Acetate Salt)

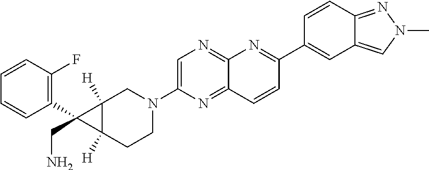 [SMILES: Cn1cc2cc(-c3ccc4nc(N5CC[C@@H]6[C@H](C5)[C@@]6(CN)c5ccccc5F)cnc4n3)ccc2n1]

The above compound was synthesized according to the procedure reported for Example 394 using Intermediate 51, 97 and Intermediate 45; Example 415 was obtained as an orange solid (TFA salt; 2.5 mg, 9%). LCMS (ES⁺) Method 2: m/z 480 (M+H)⁺, RT 1.17 min.

### Example 416: ((1S,6R,7R)-3-(3-(2,3-dichlorophenyl)imidazo[1,5-a]pyrazin-8-yl)-7-(2-fluorophenyl)-3-azabicyclo[4.1.0]heptan-7-yl)methanamine (Trifluoro Acetate Salt)

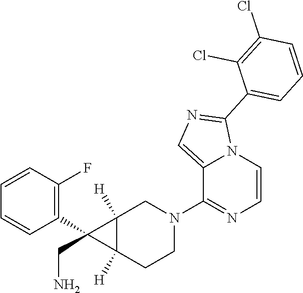 [SMILES: NC[C@]1(c2ccccc2F)[C@@H]2CCN(c3nccn4c(-c5cccc(Cl)c5Cl)ncc34)C[C@@H]21]

The above compound was synthesized according to the procedure reported for Example 2 (step 1; 1,4-dioxane at 85° C.) with Intermediate 51 and Intermediate 112 and final deprotection as step 3; Example 416 was obtained as a white solid (TFA salt; 3.2 mg, 5%). LCMS (ES⁺) Method 2: m/z 482 (M+H)⁺, RT 1.09 min.

### Example 419: ((1S,6R,7R)-3-(6-((3-fluoro-2-methylpyridin-4-yl)thio)pyrido[2,3-b]pyrazin-2-yl)-7-(2-fluorophenyl)-3-azabicyclo[4.1.0]heptan-7-yl)methanamine (Trifluoro Acetate Salt)

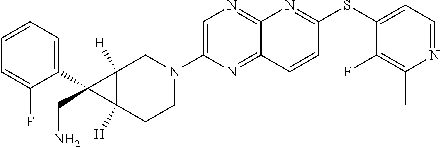 [SMILES: Cc1nccc(Sc2ccc3nc(N4CC[C@@H]5[C@H](C4)[C@@]5(CN)c4ccccc4F)cnc3n2)c1F]

The above compound was synthesized according to the procedure reported for Example 400 using Intermediate 51, 97 and Intermediate 104; Example 419 was obtained as yellow solid (TFA salt; 7 mg, 24%). ¹H NMR (400 MHz, DMSO-d₆) δ 8.93 (s, 1H), 8.32 (d, J=4.9 Hz, 1H), 8.04 (d, J=8.5 Hz, 1H), 7.66 (d, J=8.9 Hz, 1H), 7.64 (br s, 3H), 7.51-7.48 (m, 1H), 7.44 (t, J=5.2 Hz, 1H), 7.40-7.37 (m, 1H), 7.24-7.20 (m, 2H), 4.55 (d, J=14.1 Hz, 1H), 4.44-4.42 (m, 1H), 3.80 (dd, J=14.1, 6.6 Hz, 1H), 3.12-3.03 (m, 2H), 3.0-2.95 (br d, J=4.3 Hz, 1H), 2.50-2.49 (m, 3H), 2.38-2.33 (m, 1H), 1.83-1.76 (m, 2H), 1.57 (t, J=7.2 Hz, 1H). LCMS (ES⁺) Method 2: m/z 491 (M+H)⁺, RT 1.28 min.

### Example 421: 4-((5-((1S,6R,7R)-7-(aminomethyl)-7-(2-fluorophenyl)-3-azabicyclo[4.1.0]heptan-3-yl)pyrazin-2-yl)thio)-3-chlorobenzamide (Trifluoro Acetate Salt)

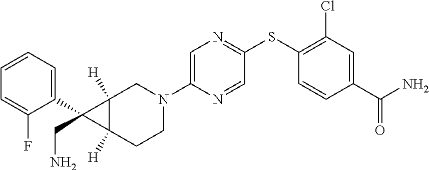 [SMILES: NC[C@]1(c2ccccc2F)[C@@H]2CCN(c3cnc(Sc4ccc(C(N)=O)cc4Cl)cn3)C[C@@H]21]

The above compound was synthesized according to the procedure reported for Example 424 using 2-ethylhexyl 3-((4-carbamoyl-2-chlorophenyl)thio)propanoate (prepared as reported in the synthesis of Intermediate 98 in step 2) in step 1 and Intermediate 51 in step 2; Example 421 was obtained as a brown solid (TFA salt; 2.0 mg, 10%). LCMS (ES⁺) Method 2: m/z 484 (M+H)⁺, RT 1.35 min.

### Example 422: ((1S,6R,7R)-7-(2-fluorophenyl)-3-(5-(quinolin-5-ylthio)pyrazin-2-yl)-3-azabicyclo[4.1.0]heptan-7-yl)methanamine (Trifluoro Acetate Salt)

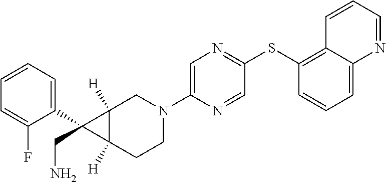 [SMILES: NC[C@]1(c2ccccc2F)[C@@H]2CCN(c3cnc(Sc4cccc5ncccc45)cn3)C[C@@H]21]

The above compound was synthesized according to the procedure reported for Example 424 using 2-ethylhexyl 3-(quinolin-5-ylthio)propanoate (prepared as reported in the synthesis of Intermediate 98 in step 2) in step 1 and Intermediate 51 in step 2; Example 422 was obtained as a yellow solid (TFA salt; 2.7 mg, 15%). LCMS (ES⁺) Method 2: m/z 458 (M+H)⁺, RT 1.41 min.

### Example 423: ((1S,6R,7R)-3-(5-((3-fluoro-2-methylpyridin-4-yl)thio)pyrazin-2-yl)-7-(2-fluorophenyl)-3-azabicyclo[4.1.0]heptan-7-yl)methanamine (Trifluoro Acetate Salt)

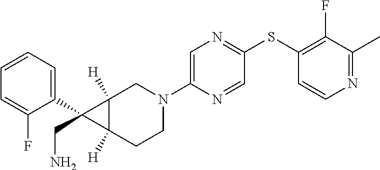 [SMILES: Cc1nccc(Sc2cnc(N3CC[C@@H]4[C@H](C3)[C@@]4(CN)c3ccccc3F)cn2)c1F]

The above compound was synthesized according to the procedure reported for Example 424 using Intermediate 51 in step 2; Example 423 was obtained as a creamy solid (TFA salt; 4.3 mg, 9%). LCMS (ES⁺) Method 2: m/z 440 (M+H)⁺, RT 1.21 min.

### Example 424: ((1S,6R,7R)-7-(2,5-difluorophenyl)-3-(5-((3-fluoro-2-methylpyridin-4-yl)thio)pyrazin-2-yl)-3-azabicyclo[4.1.0]heptan-7-yl)methanamine (Trifluoro Acetate Salt)

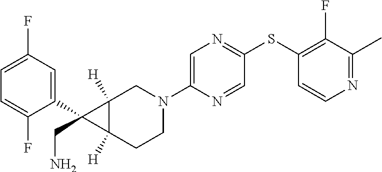 [SMILES: Cc1nccc(Sc2cnc(N3CC[C@@H]4[C@H](C3)[C@@]4(CN)c3cc(F)ccc3F)cn2)c1F]

### Step 1: 2-chloro-5-((3-fluoro-2-methylpyridin-4-yl)thio)pyrazine

A solution of sodium 3-fluoro-2-methylpyridine-4-thiolate (Intermediate 104; 51.5 mg, 0.31 mmol), 2-chloro-5-iodopyrazine (50.0 mg, 0.21 mmol), DIPEA (0.07 mL, 0.42 mmol), Pd₂(dba)₃ (4.8 mg, 0.01 mmol) and XantPhos (6.0 mg, 0.01 mmol) in 1,4-dioxane (1.0 mL) was heated at 120° C. for 1 h. The reaction mixture was diluted with EtOAc and filtered on a pad of solca floc. The solvent was concentrated in vacuo and the crude material was purified by flash chromatography (5-100% EtOAc in petroleum ether) to afford the title compound as a pale-yellow solid (46 mg, 87%). LCMS (ES⁺) Method 2: m/z 256 (M+H)⁺, RT 1.69 min.

### Step 2: ((1S,6R,7R)-7-(2,5-difluorophenyl)-3-(5-((3-fluoro-2-methylpyridin-4-yl)thio)pyrazin-2-yl)-3-azabicyclo[4.1.0]heptan-7-yl)methanamine (Trifluoro Acetate Salt)

A solution of 2-chloro-5-((3-fluoro-2-methylpyridin-4-yl)thio)pyrazine (23.0 mg, 0.09 mmol), benzyl (((1S,6R,7R)-7-(2,5-difluorophenyl)-3-azabicyclo[4.1.0]heptan-7-yl)methyl)carbamate hydrochloride (Intermediate 102; 44.1 mg, 0.11 mmol) and DIPEA (0.05 mL, 0.27 mmol) in DMSO (0.9 mL) was heated at 120° C. for 3 h. After cooling the solvent was concentrated in vacuo and the crude material was purified by flash chromatography (C18; 0-100% MeCN/H₂O+0.1% TFA) to afford the title compound as a brown solid (29 mg, 54%) which was dissolved in HBr (33% in AcOH; 1.0 mL) and the reaction mixture was stirred at rt for 1 h. The reaction mixture was concentrated in vacuo and the reaction crude was purified by preparative HPLC to obtain the title compound as white solid (TFA salt; 13 mg, 25%). LCMS (ES⁺) Method 2: m/z 458 (M+H)⁺, RT 1.20 min.

### Example 425: ((1S,6R,7R)-3-(6-(2,4-dimethyl-2H-indazol-5-yl)pyrido[2,3-b]pyrazin-2-yl)-7-(2-fluorophenyl)-3-azabicyclo[4.1.0]heptan-7-yl)methanamine (Trifluoro Acetate Salt)

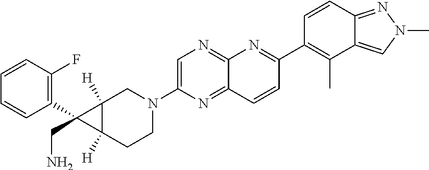 [SMILES: Cc1c(-c2ccc3nc(N4CC[C@@H]5[C@H](C4)[C@@]5(CN)c4ccccc4F)cnc3n2)ccc2nn(C)cc12]

The above compound was synthesized according to the procedure reported for Example 394 using Intermediate 51, 97 and Intermediate 53; Example 425 was obtained as an orange solid (TFA salt; 12 mg, 40%). LCMS (ES⁺) Method 2: m/z 494 (M+H)⁺, RT 1.2 min.

### Example 426: ((1S,6R,7R)-7-(2,5-difluorophenyl)-3-(6-((3-fluoro-2-methylpyridin-4-yl)thio)pyrido[2,3-b]pyrazin-2-yl)-3-azabicyclo[4.1.0]heptan-7-yl)methanamine (trifluoro acetate salt)

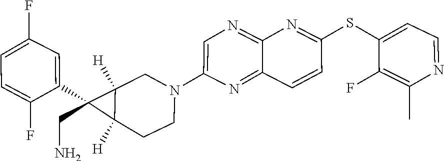 [SMILES: Cc1nccc(Sc2ccc3nc(N4CC[C@@H]5[C@H](C4)[C@@]5(CN)c4cc(F)ccc4F)cnc3n2)c1F]

The above compound was synthesized according to the procedure reported for Example 419 using Intermediate 102; Example 426 was obtained as yellow solid (TFA salt; 9 mg, 34%). ¹H NMR (400 MHz, DMSO-d₆) δ 8.91 (s, 1H), 8.30 (d, J=5.3 Hz, 1H), 8.02 (d, J=8.0 Hz, 1H), 7.67-7.64 (m, 4H), 7.43 (t, J=5.3 Hz, 1H), 7.32-7.18 (m, 3H), 4.53 (d, J=14.5 Hz, 1H), 4.43-4.39 (m, 1H), 3.75 (dd, J=14.9, 6.6 Hz, 1H), 3.12-2.93 (m, 3H), 2.48 (s, 3H), 2.35-2.39 (m, 1H), 1.85-1.75 (m, 2H), 1.61-1.58 (m, 1H). LCMS (ES⁺) Method 2: m/z 509 (M+H)⁺, RT 1.28 min.

### Example 427: ((1S,6R,7R)-3-(5-((4-chloro-2-methyl-2H-indazol-5-yl)thio)pyrazin-2-yl)-7-(2-fluorophenyl)-3-azabicyclo[4.1.0]heptan-7-yl)methanamine (Trifluoro Acetate Salt)

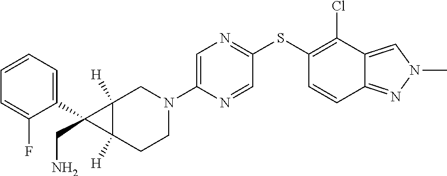 [SMILES: Cn1cc2c(Cl)c(Sc3cnc(N4CC[C@@H]5[C@H](C4)[C@@]5(CN)c4ccccc4F)cn3)ccc2n1]

A solution of sodium 4-chloro-2-methyl-2H-indazole-5-thiolate (Intermediate 110; 17.8 mg, 0.08 mmol), benzyl (((1S,6R,7R)-7-(2-fluorophenyl)-3-(5-iodopyrazin-2-yl)-3-azabicyclo[4.1.0]heptan-7-yl)methyl)carbamate (Intermediate 109; 15.0 mg, 0.03 mmol), DIPEA (9.3 uL, 0.05 mmol), Pd₂(dba)₃ (1.23 mg, 0.001 mmol) and XantPhos (1.55 mg, 0.003 mmol) in 1,4-dioxane (0.7 mL) was stirred at 95° C. for 4 h. The reaction mixture was concentrated in vacuo and the reaction crude was purified by preparative HPLC to obtain the title compound as white solid (TFA salt; 2.9 mg, 22%). LCMS (ES⁺) Method 2: m/z 495 (M+H)⁺, RT 1.33 min.

### Example 429: 3-((2-amino-3-chloropyridin-4-yl)thio)-6-((1S,6R,7R)-7-(aminomethyl)-7-(2,3-difluorophenyl)-3-azabicyclo[4.1.0]heptan-3-yl)pyrazin-2-amine (Trifluoro Acetate Salt)

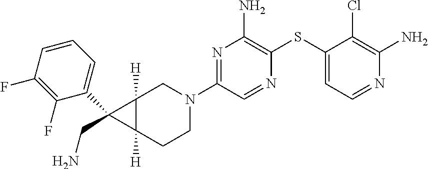 [SMILES: NC[C@]1(c2cccc(F)c2F)[C@@H]2CCN(c3cnc(Sc4ccnc(N)c4Cl)c(N)n3)C[C@@H]21]

The above example was synthesized using the procedure reported for Example 384 using Intermediates 42 and 88 (DMSO at 120° C. was used); Example 429 was obtained as a pale orange powder (TFA salt; 5.2 mg, 15%). LCMS (ES⁺) Method 2: m/z 490 (M+H)⁺, RT 0.94 min.

### Example 430: 6-((1S,6R,7R)-7-(aminomethyl)-7-(2,5-difluorophenyl)-3-azabicyclo[4.1.0]heptan-3-yl)-3-((3-chloro-2-cyclopropylpyridin-4-yl)thio)pyrazin-2-amine (Trifluoro Acetate Salt)

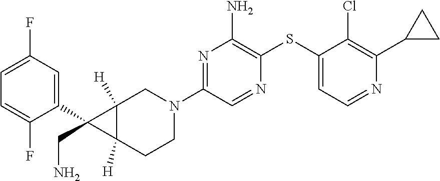 [SMILES: NC[C@]1(c2cc(F)ccc2F)[C@@H]2CCN(c3cnc(Sc4ccnc(C5CC5)c4Cl)c(N)n3)C[C@@H]21]

The above example was synthesized using the procedure reported for Example 384 using Intermediates 108 and 102; Example 384 was obtained as a yellow solid (TFA salt; 2 mg, 4%). LCMS (ES⁺) Method 2: m/z 515 (M+H)⁺, RT 1.64 min.

### Example 431: 6-((1S,6R,7R)-7-(aminomethyl)-7-(2,5-difluorophenyl)-3-azabicyclo[4.1.0]heptan-3-yl)-3-((3-fluoro-2-methylpyridin-4-yl)thio)pyrazin-2-amine (Trifluoro Acetate Salt)

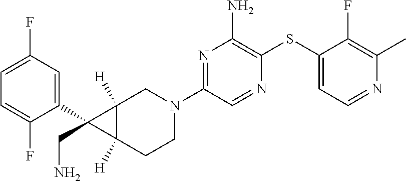 [SMILES: Cc1nccc(Sc2ncc(N3CC[C@@H]4[C@H](C3)[C@@]4(CN)c3cc(F)ccc3F)nc2N)c1F]

The above example was synthesized using the procedure reported for Example 384 using Intermediate 102 and 6-chloro-3-((3-fluoro-2-methylpyridin-4-yl)thio)pyrazin-2-amine (prepared as reported for Intermediate 108, step 3 & 4, starting from 4-bromo-3-fluoro-2-methylpyridine). The title compound was obtained as a beige solid (1 mg, 3%). LCMS (ES⁺) Method 2: m/z 473 (M+H)⁺, RT 1.12 min.

### Example 433: 6-((1R,5S,6r)-6-(aminomethyl)-6-(4-methylthiazol-2-yl)-3-azabicyclo[3.1.0]hexan-3-yl)-3-(2,3-dichlorophenyl)-2-methylpyrimidin-4(3H)-one (Trifluoro Acetate Salt)

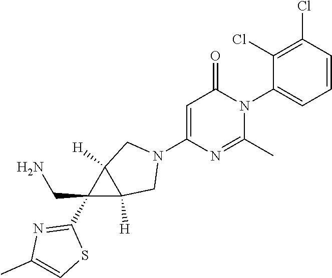 [SMILES: Cc1csc([C@@]2(CN)[C@@H]3CN(c4cc(=O)n(-c5cccc(Cl)c5Cl)c(C)n4)C[C@@H]32)n1]

The above compound was synthesized according to the procedure reported for Example 411 using Intermediate 111 and Intermediate 122, and Example 1 step 6 for the final deprotection to obtain the title compound (TFA salt, 23 mg, 39%). ¹H NMR (300 MHz, DMSO-d₆+TFA) δ 8.01 (br s, 3H), 7.86-7.75 (m, 1H), 7.61-7.45 (m, 2H), 7.22 (s, 1H), 5.10 (s, 1H), 4.14-3.59 (m, 4H), 3.29 (br s, 2H), 2.48-2.34 (m, 5H), 2.02 (s, 3H); LCMS (ES⁺) Method 2A: m/z 462 (M+H)⁺, RT 2.37 min.

The above mixture of atropoisomers was separated by chiral SFC on a Berger SFC™ MiniGram-Mettler Toledo AG using a Chiralcel® OD (2×25 cm) column (flow: 10 ml/min, T_{col} 40° C., P_{col}: 120 bar, modifier: 20% MeOH (+0.1% TEA) 3 min, 20-40% 6 min, 40% 11 min, 40-50% 3 min, 50-20% 2 min; using CO₂ as supercritic eluent); Example 433a was obtained as first eluted (RT 10.43 min) and Example 433b was obtained as second eluted (RT 11.51 min).

### Example 434: 3-((2-amino-3-chloropyridin-4-yl)thio)-6-((1R,5S,6r)-6-(aminomethyl)-6-(4-methylthiazol-2-yl)-3-azabicyclo[3.1.0]hexan-3-yl)pyrazin-2-amine (Trifluoro Acetate Salt)

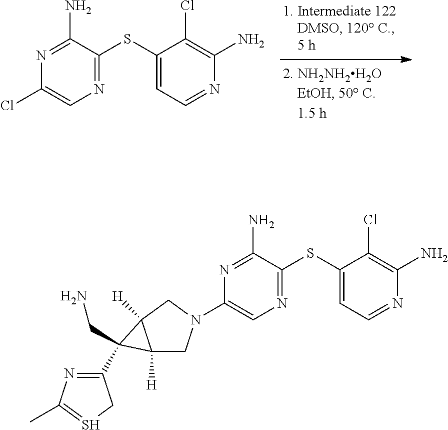

### Step 1: 2-(((1R,5S,6r)-3-(6-amino-5-((2-amino-3-chloropyridin-4-yl)thio)pyrazin-2-yl)-6-(4-methylthiazol-2-yl)-3-azabicyclo[3.1.0]hexan-6-yl)methyl)isoindoline-1,3-dione

Dry DIPEA (0.06 mL, 0.35 mmol) was added to a solution of Intermediate 42 (20 mg, 0.07 mmol) and Intermediate 122 (39 mg, 0.10 mmol) in DMSO (0.58 mL) and the reaction mixture was heated at 120° C. for 5 h. The reaction was directly purified by flash chromatography (C18, 0-40% H₂O/CH₃CN+0.1% TFA) to obtain the title compound as yellow solid (30 mg, 73%). LCMS (ES⁺) Method 1A: m/z 591 (M+H)⁺, RT 1.50 min.

### Step 2: 3-((2-amino-3-chloropyridin-4-yl)thio)-6-((1R,5S,6r)-6-(aminomethyl)-6-(4-methylthiazol-2-yl)-3-azabicyclo[3.1.0]hexan-3-yl)pyrazin-2-amine

2-(((1R,5S,6r)-3-(6-amino-5-((2-amino-3-chloropyridin-4-yl)thio)pyrazin-2-yl)-6-(4-methylthiazol-2-yl)-3-azabicyclo[3.1.0]hexan-6-yl)methyl)isoindoline-1,3-dione (30 mg, 0.05 mmol) was suspended in EtOH (3 mL) then N₂H₄·H₂O (0.1 mL, 2.0 mmol) was added and the reaction mixture was stirred at 50° C. for 1.5 h. Solvent was evaporated in vacuo and the resulting crude was purified by flash chromatography (C18, H₂O/CH₃CN+0.1% TFA) to obtain the title compound as white powder (TFA salt, 15 mg, 51%). ¹H NMR (300 MHz, DMSO-d₆+TFA) δ 8.42 (br s, 2H), 8.01 (br s, 3H), 7.76 (d, J=6.9 Hz, 1H), 7.36 (s, 1H), 7.23 (br d, J=1.0 Hz, 1H), 6.07 (d, J=6.9 Hz, 1H), 3.99-3.70 (m, 4H), 3.45-3.13 (m, 2H), 2.48-2.41 (m, 2H), 2.38 (br d, J=0.7 Hz, 3H); LCMS (ES⁺) Method 2A: m/z 461 (M+H)⁺, RT 1.69 min.

### Example 435: ((1S,6R,7R)-7-(2-fluorophenyl)-3-(6-(8-methoxyquinolin-6-yl)pyrido[2,3-b]pyrazin-2-yl)-3-azabicyclo[4.1.0]heptan-7-yl)methanamine (Trifluoro Acetate Salt)

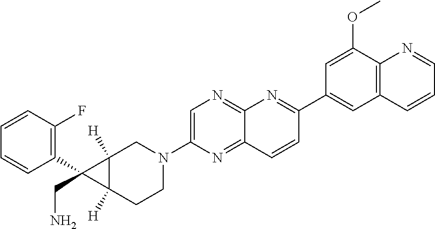 [SMILES: COc1cc(-c2ccc3nc(N4CC[C@@H]5[C@H](C4)[C@@]5(CN)c4ccccc4F)cnc3n2)cc2cccnc12]

The above example was synthesized using the procedure reported for Example 394 using Intermediate 97, 51 and (8-methoxyquinolin-5-yl)boronic acid; Example 435 was obtained as yellow solid (8 mg, 27%). LCMS (ES⁺) Method 2: m/z 507 (M+H)⁺, RT 0.85 min.

### Example 436: ((1S,6R,7R)-7-(2-fluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine (Trifluoro Acetate Salt)

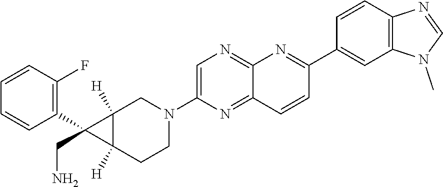 [SMILES: Cn1cnc2ccc(-c3ccc4nc(N5CC[C@@H]6[C@H](C5)[C@@]6(CN)c5ccccc5F)cnc4n3)cc21]

The above example was synthesized using the procedure reported for Example 394 using Intermediate 97, 51 and 7-chloro-1-methyl-6-(4,4,5,5-tetramethyl-1,3,2-dioxaborolan-2-yl)-1H-benzo[d]imidazole (prepared as reported for the synthesis of Intermediate 15); Example 436 was obtained as a yellow solid (2.5 mg, 9%). LCMS (ES⁺) Method 2: m/z 480 (M+H)⁺, RT 0.95 min.

### Example 437: ((1R,6S,7R)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

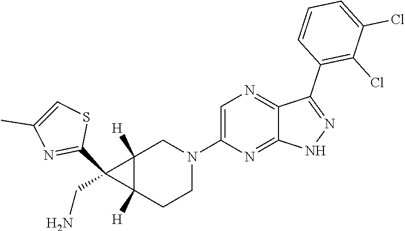 [SMILES: Cc1csc([C@@]2(CN)[C@@H]3CN(c4cnc5c(-c6cccc(Cl)c6Cl)n[nH]c5n4)CC[C@@H]32)n1]

The above example was obtained after separation of enantiomers of (3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine by chiral SFC on a Berger SFC™ MiniGram-Mettler Toledo AG using a Chiralpak® 1A (2×25 cm) column (flow: 10 ml/min, T_{col} 40° C., P_{col}: 120 bar, modifier: 25% MeOH 2 min, 25-40% 8 min, 40% 13 min, 40-25% 1 min; 25% 2 min using CO₂ as supercritic eluent). Example 438, first eluted enantiomer, was obtained as a white powder (TFA salt; 1.8 mg; 100%). LCMS (ES⁺) Method 2: m/z 486 (M+H)⁺, RT 1.29 min.

### Example 439: ((1S,6R,7S)-3-(3-(4-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

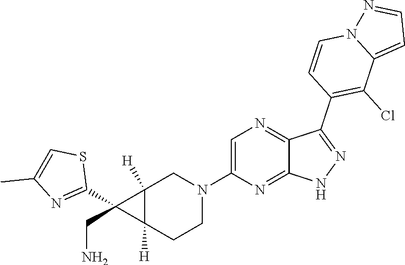 [SMILES: Cc1csc([C@@]2(CN)[C@@H]3CCN(c4cnc5c(-c6ccn7nccc7c6Cl)n[nH]c5n4)C[C@@H]32)n1]

The above example was synthesized according to the procedure described in Example 91 using Intermediate 114 and 113. Example 439 was obtained as a yellow solid (TFA salt; 5 mg, 69%). ¹H NMR (300 MHz, DMSO-d₆+TFA) δ: 8.72-8.84 (m, 1H), 8.44 (s, 1H), 8.10 (d, J=2.2 Hz, 1H), 7.92 (br s, 3H), 7.52 (d, J=7.2 Hz, 1H), 7.13 (d, J=1.0 Hz, 1H), 6.77 (br s, 1H), 4.29-4.47 (m, 2H), 3.68-3.80 (m, 1H), 3.45-3.55 (m, 1H), 3.24-3.38 (m, 1H), 2.90-3.03 (m, 1H), 2.32-2.41 (m, 4H), 1.98-2.08 (m, 1H), 1.78-1.90 (m, 2H); LCMS (ES⁺) Method 2: m/z 492, 494 (M+H)⁺, RT 2.40 min.

### Example 440: ((1S,6R,7S)-3-(3-(4-chloro-2-methylpyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine (trifluoro acetate salt)

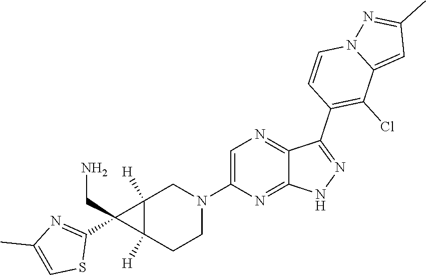

The above example was synthesized according to the procedure described in Example 77 (step 2) using Intermediate 114 and Intermediate 117 and Example 91 (step 4) for the final deprotection step; Example 440 was obtained as a yellow powder. (TFA salt; 5.5 mg, 55%). ¹H NMR (300 MHz, DMSO-d₆+TFA) δ 8.72-8.66 (dd, J=0.8, 7.2 Hz, 1H), 8.45 (s, 1H), 8.03-7.83 (br s, 3H), 7.44-7.38 (d, J=7.2 Hz, 1H), 7.19-7.16 (br d, J=1.0 Hz, 1H), 6.60 (s, 1H), 4.48-4.26 (m, 2H), 3.86-3.67 (dd, J=6.2, 14.7 Hz, 1H), 3.60-3.43 (m, 1H), 3.39-3.21 (m, 1H), 3.08-2.89 (m, 1H), 2.51 (s, 3H), 2.42-2.30 (m, 4H), 2.10-1.97 (m, 1H), 1.93-1.73 (m, 2H); LCMS (ES⁺) Method 2: m/z 506, 508 (M+H)⁺, RT 2.51 min.

### Example 442: (3-(3-(8-chloroquinolin-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

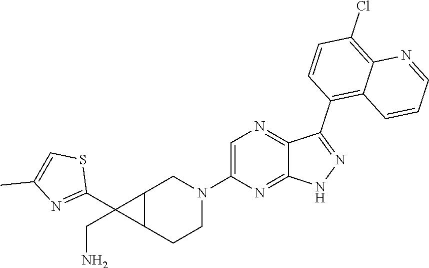 [SMILES: Cc1csc(C2(CN)C3CCN(c4cnc5c(-c6ccc(Cl)c7ncccc67)n[nH]c5n4)CC32)n1]

tert-Butyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in step 2 of the synthesis of Example 91; 12 mg, 0.02 mmol) and 8-chloro-5-(4,4,5,5-tetramethyl-1,3,2-dioxaborolan-2-yl)quinoline (prepared as reported in the synthesis of Intermediate 15, 10.7 mg, 0.04 mmol) were reacted according to the procedure described in step 2 of the synthesis of Example 77. The residue was purified by flash chromatography (30-70% EtOAc in petroleum ether) to afford the title compound as a yellow oil (12 mg, 95%). LCMS (ES⁺) Method 1: m/z 687 (M+H)⁺, RT 2.36 min. This solid was deprotected according to the procedure described in step 4 of Example 91. Example 442 was obtained as yellow solid (TFA salt; 10 mg, 95%). ¹H NMR (300 MHz, DMSO-d₆+TFA) 9.43 (dd, J=1.6, 8.7 Hz, 1H), 9.10 (dd, J=1.6, 4.2 Hz, 1H), 8.47 (s, 1H), 8.41 (d, J=8.0 Hz, 1H), 8.15 (d, J=8.0 Hz, 1H), 7.93 (br s, 3H), 7.75 (dd, J=4.2, 8.7 Hz, 1H), 7.17 (br d, J=1.0 Hz, 1H), 4.47-4.29 (m, 2H), 3.79 (dd, J=6.3, 14.8 Hz, 1H), 3.59-3.43 (m, 1H), 3.39-3.23 (m, 1H), 3.08-2.91 (m, 1H), 2.78-2.69 (m, 1H), 2.44-2.30 (m, 4H), 2.11-1.97 (m, 1H), 1.94-1.76 (m, 2H); LCMS (ES⁺) Method 2: m/z 503 (M+H)⁺, RT 2.33 min.

### Example 443: 4-(6-(7-(aminomethyl)-7-(4-methylthiazol-2-yl)-3-azabicyclo[4.1.0]heptan-3-yl)-1H-pyrazolo[3,4-b]pyrazin-3-yl)naphthalene-1-sulfonyl fluoride (Trifluoro Acetate Salt)

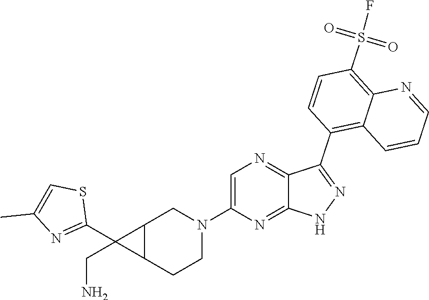 [SMILES: Cc1csc(C2(CN)C3CCN(c4cnc5c(-c6ccc(S(=O)(=O)F)c7ncccc67)n[nH]c5n4)CC32)n1]

tert-Butyl ((3-(3-iodo-1-(tetrahydro-2H-pyran-2-yl)-1H-pyrazolo[3,4-b]pyrazin-6-yl)-7-(4-methylthiazol-2-yl)-3-azabicyclo[4.1.0]heptan-7-yl)methyl)carbamate (prepared as described in step 2 of Example 91; 10 mg, 0.015 mmol) and Intermediate 118 (10.3 mg, 0.031 mmol) were reacted according to the procedure described in step 2 of Example 77. The residue was purified by flash chromatography (20-60% EtOAc in petroleum ether) to afford the title compound as a yellow oil (10 mg, 89%). LCMS (ES⁺) Method 1: m/z 734 (M+H)⁺, RT 2.73 min. This solid was deprotected according to the procedure described in step 4 of Example 91. Example 443 was obtained as yellow solid (TFA salt; 4.5 mg, 50%). ¹H NMR (300 MHz, DMSO-d₆+TFA) δ 9.06 (d, J=8.4 Hz, 1H), 8.63 (d, J=8.0 Hz, 1H), 8.57-8.39 (m, 3H), 8.06-7.77 (m, 5H), 7.18 (br d, J=0.9 Hz, 1H), 4.50-4.24 (m, 2H), 3.80 (dd, J=6.2, 14.7 Hz, 1H), 3.60-3.44 (m, 1H), 3.41-3.21 (m, 1H), 3.12-2.92 (m, 1H), 2.44-2.31 (m, 4H), 2.13-1.97 (m, 1H), 1.94-1.74 (m, 2H); LCMS (ES⁺) Method 2: m/z 550 (M+H)⁺, RT 3.07 min.

### Example 444: ((1S,6R,7S)-3-(3-(7-chloro-2-methylbenzo[d]thiazol-6-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

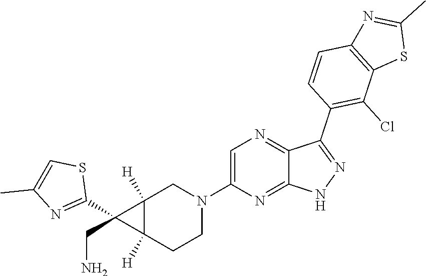

The above example was synthesized according to the procedure described in Example 77 (step 2) using Intermediate 114 and Intermediate 54 and Example 91 (step 4) for the final deprotection step. Example 444 was obtained as pale-yellow solid (TFA salt, 3.15 mg, 25% over two steps). ¹H NMR (300 MHz, DMSO-d₆+TFA) δ 8.43 (s, 1H), 7.87-8.04 (m, 5H), 7.17 (d, J=1.0 Hz, 1H), 4.29-4.43 (m, 2H), 3.70-3.80 (m, 1H), 3.47-3.56 (m, 1H), 3.27-3.37 (m, 1H), 2.92-3.03 (m, 1H), 2.87 (s, 3H), 2.35-2.41 (m, 4H), 1.98-2.08 (m, 1H), 1.78-1.89 (m, 2H); LCMS (ES⁺) Method 2: m/z 523 (M+H)⁺, RT 2.57 min.

### Example 445: ((1S,6R,7S)-3-(3-(3,4-dichloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

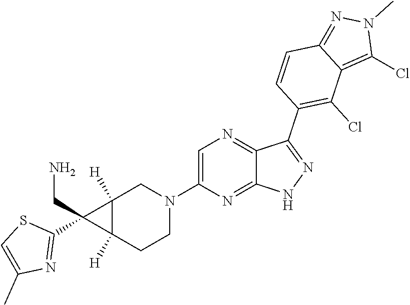 [SMILES: Cc1csc([C@@]2(CN)[C@@H]3CCN(c4cnc5c(-c6ccc7nn(C)c(Cl)c7c6Cl)n[nH]c5n4)C[C@@H]32)n1]

The above example was synthesized according to the procedure described in Example 77 (step 2) using Intermediate 114 and Intermediate 46 and Example 91 (step 4) for the final deprotection step. Example 445 was obtained as yellow solid (TFA salt; 8 mg, 62% over two steps). ¹H NMR (300 MHz, DMSO-d₆+TFA) δ 8.39 (s, 1H), 7.92 (br s, 3H), 7.68 (d, J=8.9 Hz, 1H), 7.61 (d, J=9.1 Hz, 1H), 7.15 (s, 1H), 4.46-4.28 (m, 2H), 4.16 (s, 3H), 3.77-3.68 (m, 1H), 3.56-3.47 (m, 1H), 3.37-3.27 (m, 1H), 3.02-2.89 (m, 1H), 2.36 (d, J=0.8 Hz, 4H), 2.07-1.98 (m, 1H), 1.90-1.75 (m, 2H); LCMS (ES⁺) Method 2: m/z 540, 542 (M+H)⁺, RT 2.51 min.

### Example 446: (7-(4-chlorothiazol-2-yl)-3-(3-(8-fluoroquinolin-5-yl)-1H-pyrazolo[3,4-b]pyrazin-6-yl)-3-azabicyclo[4.1.0]heptan-7-yl)methanamine (Trifluoro Acetate Salt)

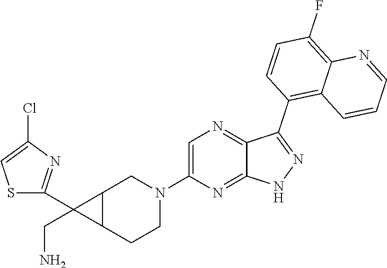 [SMILES: NCC1(c2nc(Cl)cs2)C2CCN(c3cnc4c(-c5ccc(F)c6ncccc56)n[nH]c4n3)CC21]

The above example was synthesized using the procedure reported for Example 2 using Intermediate 44 and Intermediate 119 (step 1) and 8-fluoro-5-(4,4,5,5-tetramethyl-1,3,2-dioxaborolan-2-yl)quinoline (step 2); Example 446 was obtained as yellow solid. (TFA salt; 0.6 mg, 39%). ¹H NMR (DMSO-d₆+TFA) δ 9.58 (d, J=8.7 Hz, 1H), 9.12 (dd, J=4.4, 1.4 Hz, 1H), 8.43-8.56 (m, 2H), 7.82-8.00 (m, 5H), 7.62 (s, 1H), 4.29-4.48 (m, 2H), 3.77 (dd, J=14.7, 6.1 Hz, 1H), 3.47-3.60 (m, 1H), 3.24-3.40 (m, 1H), 2.91-3.08 (m, 1H), 2.30-2.41 (m, 1H), 2.04-2.18 (m, 1H), 1.77-1.95 (m, 2H); LCMS (ES⁺) Method 2: m/z 507 (M+H)⁺, RT 2.16 min.

### Example 447: 7-((5-((1R,5S,6r)-6-(aminomethyl)-6-(4-methylthiazol-2-yl)-3-azabicyclo[3.1.0]hexan-3-yl)pyrazin-2-yl)thio)-8-chloro-N,N-dimethylimidazo[1,2-a]pyridine-2-carboxamide (trifluoro acetate salt)

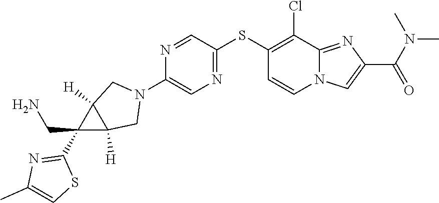

The above compound was synthesized according to the procedure reported for Example 71 using Intermediate 70, Intermediate 75, (DMA and K₂CO₃ were used). The title compound was obtained as white powder (TFA salt, 1.3 mg, 6%). LCMS (ES⁺) Method 2A: m/z 541 (M+H)⁺, RT 2.07 min

### Example 448: 5-(6-((1S,6R,7S)-7-(aminomethyl)-7-(4-methylthiazol-2-yl)-3-azabicyclo[4.1.0]heptan-3-yl)-1H-pyrazolo[3,4-b]pyrazin-3-yl)quinolin-8-yl sulfurofluoridate (Trifluoro Acetate Salt)

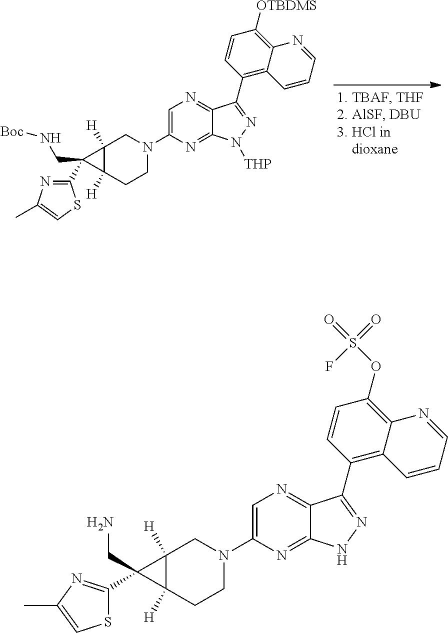

Tert-butyl (((1S,6R,7S)-3-(3-(8-((tert-butyldimethylsilyl)oxy)quinolin-5-yl)-1-(tetrahydro-2H-pyran-2-yl)-1H-pyrazolo[3,4-b]pyrazin-6-yl)-7-(4-methylthiazol-2-yl)-3-azabicyclo [4.1.0]heptan-7-yl)methyl)carbamate (prepared according to the procedure described in Example 77 (step 2) using Intermediate 114 and Intermediate 120; 15 mg, 0.02 mmol) was dissolved in THF (1 mL) and TBAF (1M sol. in THF, 0.045 mL, 0.045 mmol) was added. The resulting dark-red solution was stirred at rt for 1 h, then H₂O and NH₄Cl sat. sol. were added and the mixture was extracted with EtOAc. The organic layer was dried over Na₂SO₄, filtered, and concentrated in vacuo to obtain the title compound as a yellow oil (15 mg). The residue was dissolved in dry THF (1 mL), cooled to 0° C. then AISF (6.6 mg, 0.021 mmol) and DBU (0.010 mL, 0.07 mmol) were added. The reaction mixture was stirred at 0° C. for 1 h then EtOAc and H₂O were added, the organic phase was isolated and washed with 0.5 N HCl sol., brine, dried over Na₂SO₄, filtered, and concentrated in vacuo to obtain crude title compound (16 mg; LCMS (ES⁺) Method 1: m/z 751 (M+H)⁺, RT 2.57 min.) which was dissolved in DCM (0.5 mL) and treated with HCl (0.1 mL; 4N in 1,4-dioxane). The solution was stirred at rt for 1 h, then the solvent was removed in vacuo. The resulting crude was purified by preparative HPLC to afford the title compound as a light-yellow solid (TFA salt; 6 mg, 46% over 3 steps). ¹H NMR (300 MHz, DMSO-d₆+TFA) δ 9.46-9.39 (dd, J=1.5, 8.7 Hz, 1H), 9.18-9.12 (dd, J=1.5, 4.2 Hz, 1H), 8.56-8.47 (m, 2H), 8.29-8.21 (d, J=8.2 Hz, 1H), 8.02-7.86 (br s, 3H), 7.84-7.76 (dd, J=4.2, 8.7 Hz, 1H), 7.20-7.17 (br d, J=1.0 Hz, 1H), 4.48-4.29 (m, 2H), 3.88-3.74 (m, 1H), 3.60-3.45 (m, 1H), 3.39-3.21 (m, 1H), 3.10-2.92 (m, 1H), 2.43-2.31 (m, 4H), 2.11-1.98 (m, 1H), 1.93-1.74 (m, 2H); LCMS (ES⁺) Method 2: m/z 567 (M+H)⁺, RT 2.77 min.

### Example 449: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl sulfurofluoridate (Trifluoro Acetate Salt)

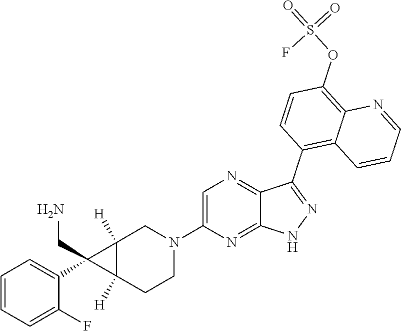 [SMILES: NC[C@]1(c2ccccc2F)[C@@H]2CCN(c3cnc4c(-c5ccc(OS(=O)(=O)F)c6ncccc56)n[nH]c4n3)C[C@@H]21]

The above example was synthesized according to the procedure described in Example 448 using Intermediate 116 and Intermediate 120. Example 449 was obtained as a light-yellow powder (TFA salt, 1.5 mg, 8% over 3 steps). Method 2: m/z 564 (M+H)⁺, RT 2.87 min.

### Example 450: 4-(6-(7-(aminomethyl)-7-(4-methylthiazol-2-yl)-3-azabicyclo[4.1.0]heptan-3-yl)-1H-pyrazolo[3,4-b]pyrazin-3-yl)-2,3-dichlorophenyl sulfurofluoridate (Trifluoro Acetate Salt)

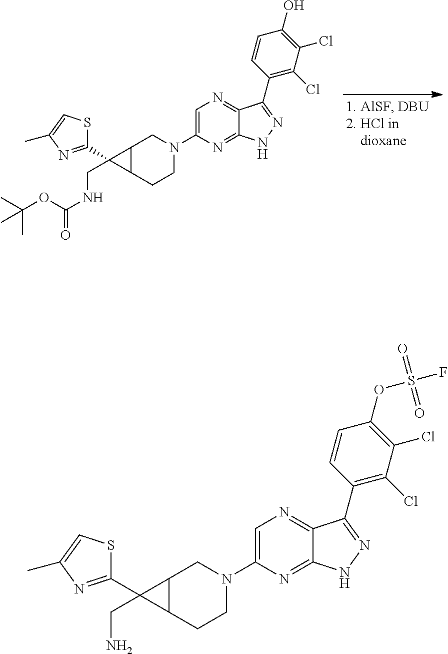 [SMILES: Cc1csc([C@@]2(CNC(=O)OC(C)(C)C)C3CCN(c4cnc5c(-c6ccc(O)c(Cl)c6Cl)n[nH]c5n4)CC32)n1 ; Cc1csc(C2(CN)C3CCN(c4cnc5c(-c6ccc(OS(=O)(=O)F)c(Cl)c6Cl)n[nH]c5n4)CC32)n1]

The above example was synthesized according to the procedure described in Example 448 (step 2 and 3) using Intermediate 114 and tert-butyl(2,3-dichloro-4-(4,4,5,5-tetramethyl-1,3,2-dioxaborolan-2-yl)phenoxy)dimethylsilane). Example 450 was obtained as white powder (TFA salt; 3.41 mg, 46%). ¹H NMR (300 MHz, DMSO-d₆+TFA) δ 8.40 (s, 1H), 7.87-8.01 (m, 5H), 7.14 (s, 1H), 4.28-4.44 (m, 2H), 3.68-3.80 (m, 1H), 3.45-3.57 (m, 1H), 3.25-3.38, (m, 1H), 2.91-3.03 (m, 1H), 2.32-2.42 (m, 4H), 1.97-2.09 (m, 1H), 1.75-1.90 (m, 2H); LCMS (ES⁺) Method 2: m/z 584 (M+H)⁺, RT 3.04 min.

### Example 451: ((1S,6R,7R)-3-(3-(3-chloro-2-(1H-pyrazol-1-yl)pyridin-4-yl)-1H-pyrazolo[3,4-b]pyrazin-6-yl)-7-(2-fluorophenyl)-3-azabicyclo[4.1.0]heptan-7-yl)methanamine (trifluoro acetate salt)

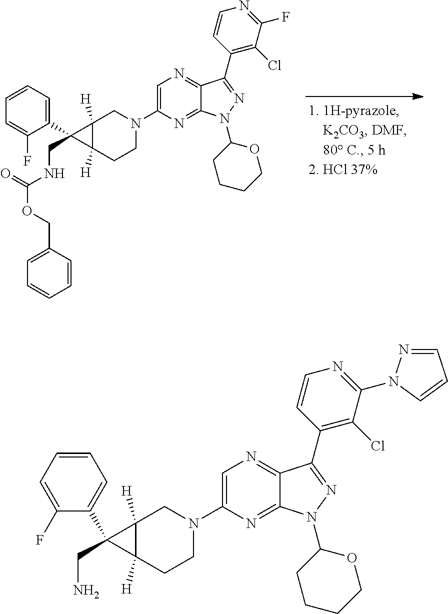 [SMILES: O=C(NC[C@]1(c2ccccc2F)[C@@H]2CCN(c3cnc4c(-c5ccnc(F)c5Cl)nn(C5CCCCO5)c4n3)C[C@@H]21)OCc1ccccc1 ; NC[C@]1(c2ccccc2F)[C@@H]2CCN(c3cnc4c(-c5ccnc(-n6cccn6)c5Cl)nn(C5CCCCO5)c4n3)C[C@@H]21]

A mixture of benzyl (((1S,6R,7R)-3-(3-(3-chloro-2-fluoropyridin-4-yl)-1-(tetrahydro-2H-pyran-2-yl)-1H-pyrazolo[3,4-b]pyrazin-6-yl)-7-(2-fluorophenyl)-3-azabicyclo[4.1.0]heptan-7-yl)methyl)carbamate (synthesized using the procedure reported for Example 2 using Intermediates 44, 51 and (3-chloro-2-fluoropyridin-4-yl)boronic acid; 5 mg, 0.010 mmol), 1H-pyrazole and K₂CO₃ in DMF (3 ml) was stirred at 80° C. for 5 h. The mixture was treated with 5% citric acid sol. and extracted with EtOAc (3×). The collected organic layers were washed with brine, dried over Na₂SO₄ filtered, concentrated and the resulting crude (5 mg) was deprotected with the procedure of Example 2 (step 3). Example 451 was obtained as a white solid (TFA salt; 0.85 mg, 19% over 2 steps). LCMS (ES⁺) Method 2: m/z 516 (M+H)⁺, RT 2.37 min.

### Example 452: ((1R,5S,6s)-3-(3-(2,3-dichlorophenyl)-1H-pyrazolo[3,4-b]pyrazin-6-yl)-6-phenyl-3-azabicyclo[3.1.0]hexan-6-yl)methanamine (Trifluoro Acetate Salt)

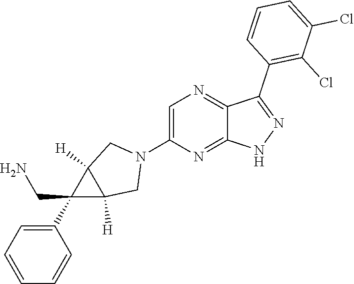 [SMILES: NC[C@]1(c2ccccc2)[C@@H]2CN(c3cnc4c(-c5cccc(Cl)c5Cl)n[nH]c4n3)C[C@@H]21]

The above example was synthesized using the procedure reported in the Example 2 using Intermediate 44, Intermediate 121 and (2,3-dichlorophenyl)boronic acid; Example 452 was obtained as a light-yellow powder (TFA salt; 12 mg, 59%). ¹H NMR (300 MHz, DMSO-d₆+TFA) δ 8.10 (s, 1H), 7.82-7.61 (m, 5H), 7.49 (s, 3H), 7.45-7.37 (m, 2H), 7.37-7.28 (m, 1H), 4.06-3.82 (m, 4H), 3.18-3.00 (m, 2H), 2.39-2.30 (m, 2H); LCMS (ES⁺) Method 2: m/z 451 (M+H)⁺, RT 2.63 min.

### Example 453: ((1R,5S,6s)-3-(3-(4-chloro-2-methyl-2H-indazol-5-yl)-1H-pyrazolo[3,4-b]pyrazin-6-yl)-6-phenyl-3-azabicyclo[3.1.0]hexan-6-yl)methanamine (Trifluoro Acetate Salt)

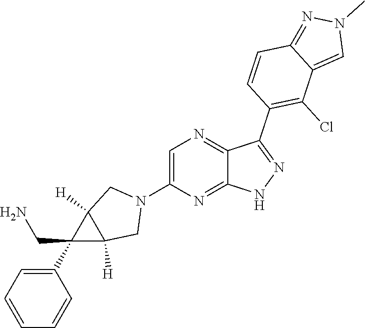 [SMILES: Cn1cc2c(Cl)c(-c3n[nH]c4nc(N5C[C@@H]6[C@H](C5)[C@@]6(CN)c5ccccc5)cnc34)ccc2n1]

The above example was synthesized using the procedure reported in the Example 2 using Intermediate 44 and Intermediate 121, and then Intermediate 45; Example 453 was obtained as a yellow powder (TFA salt; 7 mg, 43%). ¹H NMR (300 MHz, DMSO-d₆+TFA) δ 8.55 (s, 1H), 8.10 (s, 1H), 7.86-7.59 (m, 5H), 7.56-7.47 (m, 2H), 7.46-7.38 (m, 2H), 7.37-7.29 (m, 1H), 4.24 (s, 3H), 4.04-3.82 (m, 4H), 3.19-3.00 (m, 2H), 2.38-2.30 (m, 2H); LCMS (ES⁺) Method 2: m/z 471 (M+H)⁺, RT 2.21 min.

### Example 454: ((1S,6R,7R)-3-(3-(2-chloro-4-(1H-pyrrol-1-yl)phenyl)-1H-pyrazolo[3,4-b]pyrazin-6-yl)-7-(2-fluorophenyl)-3-azabicyclo[4.1.0]heptan-7-yl)methanamine (Trifluoro Acetate Salt)

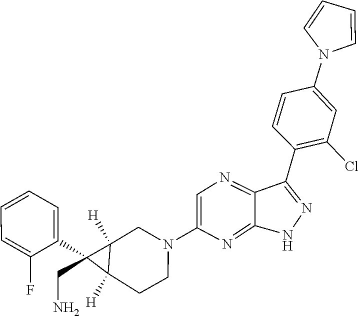 [SMILES: NC[C@]1(c2ccccc2F)[C@@H]2CCN(c3cnc4c(-c5ccc(-n6cccc6)cc5Cl)n[nH]c4n3)C[C@@H]21]

The above example was synthesized according to the procedure described in Example 77 (step 2) using Intermediate 116 and Intermediate 135 and Example 91 (step 4) for the final deprotection step. Example 454 was obtained as yellow powder (TFA salt, 5 mg, 23%). ¹H NMR (300 MHz, DMSO-d₆+TFA) δ 8.44 (s, 1H), 7.93-7.83 (m, 2H), 7.77-7.60 (m, 4H), 7.57-7.45 (m, 3H), 7.42-7.31 (m, 1H), 7.27-7.14 (m, 2H), 6.32 (br t, J=2.2 Hz, 2H), 4.46 (br d, J=14.1 Hz, 1H), 4.40-4.22 (m, 1H), 3.72 (dd, J=6.4, 14.3 Hz, 1H), 3.25-3.02 (m, 2H), 3.01-2.86 (m, 1H), 2.42-2.29 (m, 1H), 1.88-1.71 (m, 2H), 1.64-1.48 (m, 1H); LCMS (ES⁺) Method 2: m/z 514 (M+H)⁺, RT 2.95 min.

### Example 455: ((1R,5S,6s)-3-(3-(8-fluoroquinolin-6-yl)-1H-pyrazolo[3,4-b]pyrazin-6-yl)-6-phenyl-3-azabicyclo[3.1.0]hexan-6-yl)methanamine (Trifluoro Acetate Salt)

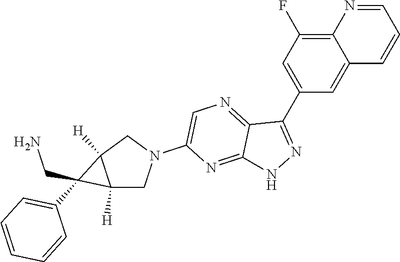 [SMILES: NC[C@]1(c2ccccc2)[C@@H]2CN(c3cnc4c(-c5cc(F)c6ncccc6c5)n[nH]c4n3)C[C@@H]21]

The above example was synthesized using the procedure reported in the Example 2 using Intermediate 44 and Intermediate 121 and then Intermediate 86. Example 455 was obtained as a yellow powder. ¹H NMR (300 MHz, DMSO-d₆+TFA) δ 9.05 (dd, J=4.5, 1.4 Hz, 1H), 8.92 (s, 1H), 8.78 (d, J=8.5 Hz, 1H), 8.55 (dd, J=12.1, 1.5 Hz, 1H), 8.23 (s, 1H), 7.81 (dd, J=8.4, 4.5 Hz, 1H), 7.72 (br s, 3H), 7.48-7.54 (m, 2H), 7.41 (t, J=7.4 Hz, 2H), 7.28-7.36 (m, 1H), 3.92-4.02 (m, 4H), 3.06-3.16 (m, 2H), 2.34 (br s, 2H); LCMS (ES⁺) Method 2: m/z 452 (M+H)⁺, RT 2.29 min.

### Example 456: ((1R,5S,6s)-3-(3-(5-chloroquinoxalin-6-yl)-1H-pyrazolo[3,4-b]pyrazin-6-yl)-6-phenyl-3-azabicyclo[3.1.0]hexan-6-yl)methanamine (Trifluoro Acetate Salt)

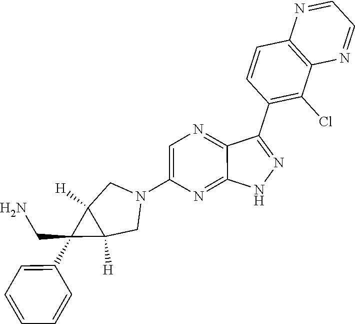 [SMILES: NC[C@]1(c2ccccc2)[C@@H]2CN(c3cnc4c(-c5ccc6nccnc6c5Cl)n[nH]c4n3)C[C@@H]21]

The above example was synthesized using the procedure reported in the Example 2 using Intermediate 44 and Intermediate 121 and then Intermediate 82. Example 456 was obtained as a yellow powder (TFA salt, 4 mg, 29%). ¹H NMR (300 MHz, DMSO-d₆+TFA) δ 9.12 (dd, J=1.7, 12.0 Hz, 2H), 8.23 (dd, J=8.7, 14.2 Hz, 2H), 8.15 (s, 1H), 7.71 (br s, 3H), 7.56-7.47 (m, 2H), 7.47-7.37 (m, 2H), 7.37-7.27 (m, 1H), 4.13-3.77 (m, 4H), 3.19-3.02 (m, 2H), 2.34 (br s, 2H); LCMS (ES⁺) Method 2: m/z 469 (M+H)⁺, RT 2.16 min.

### Example 457: ((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)methanamine (Trifluoro Acetate Salt)

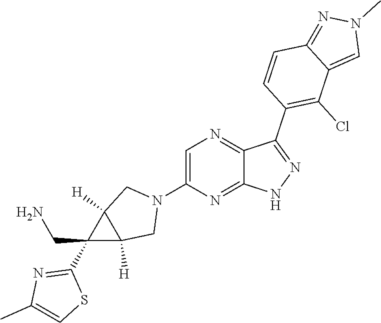

The above example was synthesized using the procedure reported for Example 2 using Intermediates 44, 124 and 45; Example 457 was obtained as a pale-yellow powder (TFA salt; 8 mg, 39%). ¹H NMR (300 MHz, DMSO-d₆+TFA) δ 8.55 (s, 1H), 8.08 (s, 1H), 7.98 (br s, 3H), 7.72-7.57 (m, 2H), 7.24 (br d, J=1.0 Hz, 1H), 4.24 (s, 3H), 4.11-3.88 (m, 4H), 3.41-3.21 (m, 2H), 2.54-2.49 (m, 2H), 2.39 (br d, J=0.9 Hz, 3H); LCMS (ES⁺) Method 2: m/z 492 (M+H)⁺, RT 2.13 min.

### Example 468: ((1R,5S,6r)-3-(3-(4-chloro-2-(difluoromethyl)-2H-indazol-5-yl)-1H-pyrazolo[3,4-b]pyrazin-6-yl)-6-(isothiazol-3-yl)-3-azabicyclo[3.1.0]hexan-6-yl)methanamine (trifluoro acetate salt)

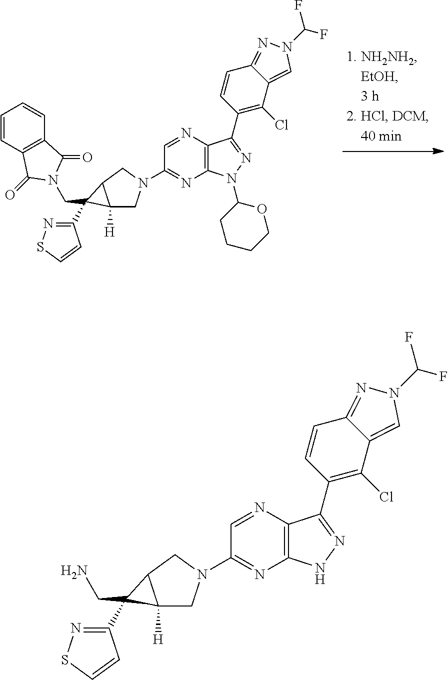

2-(((1R,5S,6r)-3-(3-(4-chloro-2-(difluoromethyl)-2H-indazol-5-yl)-1-(tetrahydro-2H-pyran-2-yl)-1H-pyrazolo[3,4-b]pyrazin-6-yl)-6-(isothiazol-3-yl)-3-azabicyclo[3.1.0]hexan-6-yl)methyl)isoindoline-1,3-dione (synthesized according to the procedure reported for Example 2 using Intermediate 44, Intermediate 133 and Intermediate 137c; 18 mg, 0.02 mmol) was suspended in EtOH (1.2 mL) then N₂H₄·H₂O (0.04 mL, 0.74 mmol) was added. The reaction mixture was stirred at rt for 3 h then the solvent was concentrated in vacuo. The residue was taken up in DCM (1.2 mL) and 4N HCl in dioxane (0.19 mL, 0.76 mmol) was added. The reaction mixture was stirred at rt for 40 min, then the solvent was concentrated in vacuo. The reaction was purified by reverse phase chromatography (C18; 0-30% MeCN/H₂O+0.1% TFA) to obtain the title compound as yellow solid (TFA salt; 7.5 mg, 48%). ¹H NMR (300 MHz, DMSO-d₆+TFA) δ 9.16-9.04 (m, 2H), 8.41-7.88 (m, 5H), 7.83 (s, 2H), 7.27 (d, J=4.9 Hz, 1H), 4.13-3.81 (m, 4H), 3.46-3.22 (m, 2H), 2.61-2.55 (m, 2H); LCMS (ES⁺) Method 2A: m/z 514 (M+H)⁺, RT 2.29 min.

### Example 469: 6-((1R,5S,6r)-6-(aminomethyl)-6-(4-methylthiazol-2-yl)-3-azabicyclo[3.1.0]hexan-3-yl)-3-(7-chloro-2-methylbenzo[d]thiazol-6-yl)-5-methyl-1,5-dihydro-4H-pyrazolo[3,4-d]pyrimidin-4-one (Trifluoro Acetate Salt)

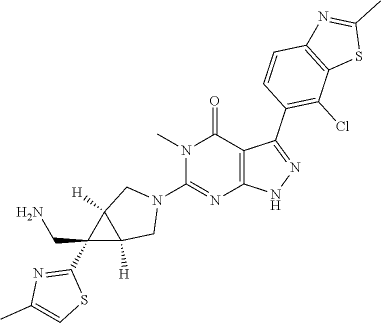

Intermediate 47 (57 mg, 0.15 mmol) and Intermediate 122 (50 mg, 0.13 mmol) were reacted according to the procedure described in Example 62 (step 1) using DMSO, to obtain a beige solid (95 mg; LCMS (ES⁺) Method 1A: m/z 698 (M+H)⁺, RT 2.12 min). This crude (25 mg, 0.036 mmol) and Intermediate 54 (22 mg, 0.07 mol) were reacted according to the procedure described in Example 62 (step 2) to afford a yellow oil (21 mg, 77%; LCMS (ES⁺) Method 1A: m/z 753 (M+H)⁺, RT 2.34 min), which was suspended in EtOH (1 mL). N₂H₄·H₂O (0.04 mL, 0.84 mmol) was added, and the reaction mixture was stirred at rt for 4 h, then the solvent was concentrated in vacuo. The residue was taken up in DCM (1 mL) and 4N HCl in dioxane (0.21 mL, 0.84 mmol) was added. The mixture was stirred at rt for 30 min, then the solvent was concentrated in vacuo. The resulting crude was purified by preparative HPLC to obtain the title product as a white solid (TFA salt, 4 mg, 22%). ¹H NMR (300 MHz, DMSO-d₆+TFA) δ 8.05-7.89 (m, 4H), 7.63 (d, J=8.3 Hz, 1H), 7.22 (br d, J=1.0 Hz, 1H), 4.19-3.94 (m, 4H), 3.49-3.40 (m, 2H), 3.38 (s, 3H), 2.87 (s, 3H), 2.44-2.39 (m, 2H), 2.38 (br d, J=0.8 Hz, 3H). LCMS (ES⁺) Method 2A: m/z 539 (M+H)⁺, RT 2.35 min.

### Example 470: 6-((1R,5S,6r)-6-(aminomethyl)-6-(4-chlorothiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one (Trifluoro Acetate Salt)

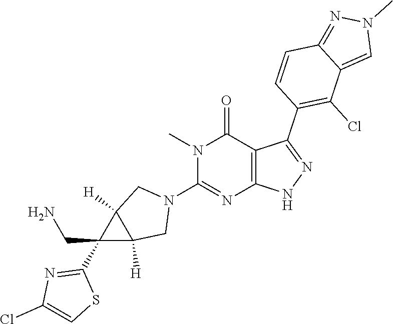 [SMILES: Cn1cc2c(Cl)c(-c3n[nH]c4nc(N5C[C@@H]6[C@H](C5)[C@@]6(CN)c5nc(Cl)cs5)n(C)c(=O)c34)ccc2n1]

The above compound was synthesized according to the procedure reported for Example 469 using Intermediate 47, Intermediate 125 and Intermediate 45 using HCl in the final deprotection step. Example 470 was obtained as a white powder (TFA salt, 2 mg, 34%). ¹H NMR (300 MHz, DMSO-d₆+TFA) δ 8.49 (s, 1H), 7.98 (br s, 3H), 7.67 (s, 1H), 7.59 (dd, J=8.8, 0.8 Hz, 1H), 7.32 (d, J=8.9 Hz, 1H), 4.22 (s, 3H), 3.98-4.14 (m, 4H), 3.42-3.52 (m, 2H), 3.37 (s, 3H), 2.46 (br s, 2H). LCMS (ES⁺) Method 2A: m/z 542 (M+H)⁺, RT 2.09 min.

### Example 471: 6-((1R,5S,6r)-6-(aminomethyl)-6-(4-(trifluoromethyl)thiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one (Trifluoro Acetate Salt)

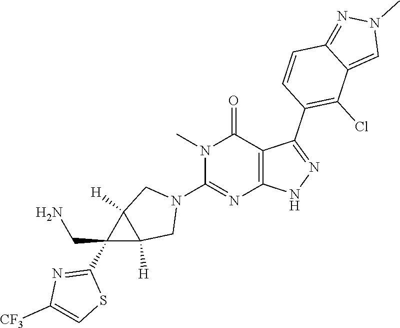 [SMILES: Cn1cc2c(Cl)c(-c3n[nH]c4nc(N5C[C@@H]6[C@H](C5)[C@@]6(CN)c5nc(C(F)(F)F)cs5)n(C)c(=O)c34)ccc2n1]

The above compound was synthesized according to the procedure reported for Example 469 using Intermediate 47, Intermediate 140 and Intermediate 45; Example 471 was obtained as a white solid (TFA salt; 7 mg, 53%). ¹H NMR (300 MHz, DMSO-d₆+TFA) δ 8.55-8.43 (m, 2H), 7.99 (br s, 3H), 7.60 (dd, J=0.7, 8.8 Hz, 1H), 7.33 (d, J=8.8 Hz, 1H), 4.23 (s, 3H), 4.16-4.00 (m, 4H), 3.57-3.44 (m, 2H), 3.38 (s, 3H), 2.58-2.54 (m, 2H); LCMS (ES⁺) Method 2A: m/z 576 (M+H)⁺, RT 2.21 min.

### Example 472: 6-((1R,5S,6r)-6-(aminomethyl)-6-(4-methylthiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one (Trifluoro Acetate Salt)

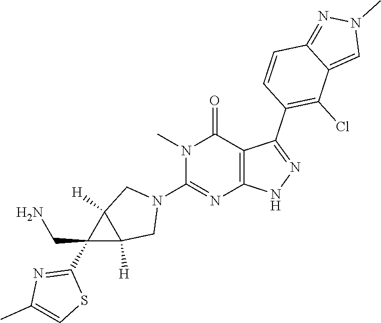

The above compound was synthesized according to the procedure reported for Example 469 using Intermediate 47, Intermediate 122 and Intermediate 45. Example 472 was obtained as a white solid (TFA salt; 7 mg, 45%). ¹H NMR (300 MHz, DMSO-d₆+TFA) δ 8.52 (s, 1H), 7.98 (br s, 3H), 7.60 (dd, J=0.8, 8.8 Hz, 1H), 7.32 (d, J=8.8 Hz, 1H), 7.23 (br d, J=1.0 Hz, 1H), 4.23 (s, 3H), 4.16-3.96 (m, 4H), 3.50-3.40 (m, 2H), 3.37 (s, 3H), 2.43-2.39 (m, 2H), 2.38 (br d, J=0.8 Hz, 3H); LCMS (ES⁺) Method 2A: m/z 522 (M+H)⁺, RT 2.08 min.

### Example 473: 2-((1R,5S,6r)-6-(aminomethyl)-6-(4-methylthiazol-2-yl)-3-azabicyclo[3.1.0]hexan-3-yl)-5-(4-chloro-2-methyl-2H-indazol-5-yl)-3-methyl-3,7-dihydro-4H-pyrrolo[2,3-d]pyrimidin-4-one (Trifluoro Acetate Salt)

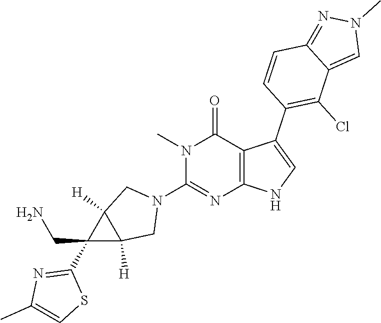

The above compound was synthesized using the procedure reported for Example 469 using Intermediate 96, Intermediate 124 and Intermediate 45. TFA followed by treatment with NH₃ was used in the final deprotection step. Example 473 was obtained as an off-white powder (TFA salt; 3.75 mg, 31%). ¹H NMR (300 MHz, DMSO-d₆+TFA) δ 11.61 (d, J=1.8 Hz, 1H), 8.38 (s, 1H), 8.05-7.92 (m, 3H), 7.49 (dd, J=0.7, 8.8 Hz, 1H), 7.34 (d, J=8.8 Hz, 1H), 7.20 (d, J=1.0 Hz, 1H), 6.97 (d, J=2.4 Hz, 1H), 4.19 (s, 3H), 4.06-3.90 (m, 3H), 3.52 (br d, J=5.8 Hz, 2H), 3.37 (s, 3H), 2.40-2.35 (m, 5H). LCMS (ES⁺) Method 2A: m/z 521 (M+H)⁺, RT 2.29 min.

### Example 474: ((1R,5S,6r)-3-(3-(4-chloro-2-methyl-2H-indazol-5-yl)-1H-pyrazolo[3,4-b]pyrazin-6-yl)-6-(1-methyl-1H-pyrazol-3-yl)-3-azabicyclo[3.1.0]hexan-6-yl)methanamine (Trifluoro Acetate Salt)

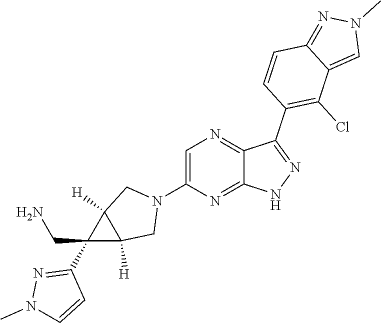

The above compound was synthesized using the procedure reported for Example 2 using Intermediate 44, Intermediate 127 and Intermediate 45. Example 474 was obtained as a light-yellow powder (TFA salt, 7 mg, 41%). ¹H NMR (300 MHz, DMSO-d₆+TFA) δ 8.55 (s, 1H), 8.07 (s, 1H), 7.84 (br s, 3H), 7.73-7.61 (m, 3H), 6.12 (d, J=2.2 Hz, 1H), 4.24 (s, 3H), 3.99-3.87 (m, 4H), 3.83 (s, 3H), 3.27-3.10 (m, 2H), 2.36-2.25 (m, 2H); LCMS (ES⁺) Method 2A: m/z 475 (M+H)⁺, RT 1.98 min.

### Example 475: ((1R,5S,6r)-3-(3-(7-chloro-2-methylbenzo[d]thiazol-6-yl)-1H-pyrazolo[3,4-b]pyrazin-6-yl)-6-(1-methyl-1H-pyrazol-3-yl)-3-azabicyclo[3.1.0]hexan-6-yl)methanamine (trifluoro acetate salt)

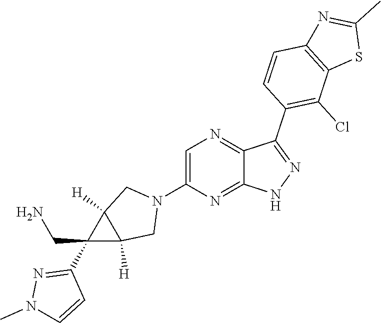

The above compound was synthesized using the procedure reported for Example 2 using Intermediate 44, Intermediate 127 and Intermediate 54. Example 475 was obtained as an off-white solid (TFA salt; 7 mg, 43%). ¹H NMR (300 MHz, DMSO-d₆+TFA) δ 8.09 (s, 1H), 7.99 (dd, J=8.4, 19.6 Hz, 2H), 7.84 (br s, 3H), 7.68 (d, J=2.3 Hz, 1H), 6.12 (d, J=2.3 Hz, 1H), 4.06-3.86 (m, 4H), 3.83 (s, 3H), 3.29-3.08 (m, 2H), 2.87 (s, 3H), 2.37-2.26 (m, 2H); LCMS (ES⁺) Method 2A: m/z 492 (M+H)⁺, RT 2.28 min.

### Example 481: ((1S,6R,7S)-3-(3-(6-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine (Trifluoro Acetate Salt)

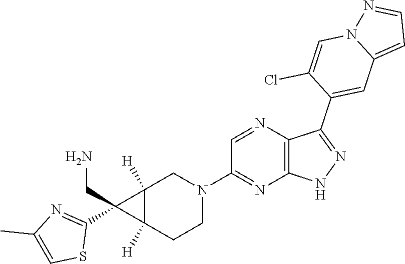 [SMILES: Cc1csc([C@@]2(CN)[C@@H]3CCN(c4cnc5c(-c6cc7ccnn7cc6Cl)n[nH]c5n4)C[C@@H]32)n1]

The above example was synthesized according to the procedure described in Example 77 (step 2) using Intermediate 114 and Intermediate 134, and Example 91 (step 4) for the final deprotection step; Example 481 was obtained as a light-yellow powder (TFA salt, 8 mg, 53%). ¹H NMR (300 MHz, DMSO-d₆+TFA) δ 9.14 (s, 1H), 8.44 (s, 1H), 8.23 (s, 1H), 8.10 (d, J=2.2 Hz, 1H), 7.92 (br s, 3H), 7.17 (br d, J=1.0 Hz, 1H), 6.80 (br dd, J=0.7, 2.2 Hz, 1H), 4.49-4.22 (m, 2H), 3.76 (dd, J=6.2, 14.6 Hz, 1H), 3.61-3.42 (m, 1H), 3.40-3.22 (m, 1H), 3.08-2.88 (m, 1H), 2.42-2.30 (m, 4H), 2.10-1.97 (m, 1H), 1.92-1.74 (m, 2H); LCMS (ES⁺) Method 2A: m/z 492 (M+H)⁺, RT 2.33 min.

### Example 484: 1-((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)-N-methylmethanamine (trifluoro acetate salt)

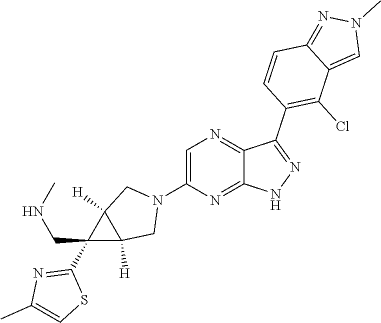

The above example was synthesized according to the procedure described in Example 77 (step 2) using Intermediate 145 and Intermediate 45, and Example 91 (step 4) for the final deprotection step; Example 484 was obtained as an off-white powder (TFA salt, 9 mg, 59%). ¹H NMR (300 MHz, DMSO-d₆+TFA) δ 8.70-8.50 (m, 3H), 8.08 (s, 1H), 7.72-7.58 (m, 2H), 7.26 (br d, J=0.9 Hz, 1H), 4.24 (s, 3H), 4.06-3.90 (m, 4H), 3.50-3.38 (m, 2H), 2.64 (t, J=5.3 Hz, 3H), 2.57-2.53 (m, 2H), 2.40 (br d, J=0.8 Hz, 3H). LCMS (ES⁺) Method 2A: m/z 506 (M+H)⁺, RT 2.20 min.

### Example 485: ((1R,5S,6r)-3-(3-(4-chloro-2-methyl-2H-indazol-5-yl)-1H-pyrazolo[3,4-b]pyrazin-6-yl)-6-(2-methylthiazol-4-yl)-3-azabicyclo[3.1.0]hexan-6-yl)methanamine (Trifluoro Acetate Salt)

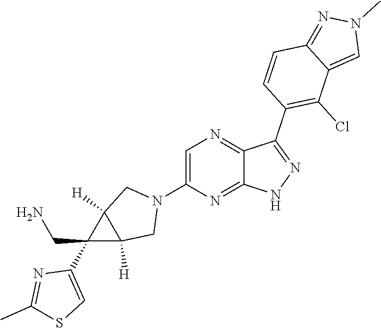

The above example was synthesized using the procedure reported for Example 2 using Intermediates 44, 150 and 45. Example 485 was obtained as a pale-yellow powder. (TFA salt, 8 mg, 65%). ¹H NMR (300 MHz, DMSO-d₆+TFA) δ 8.53 (s, 1H), 8.08 (s, 1H), 7.88 (br s, 3H), 7.60-7.72 (m, 2H), 7.34 (s, 1H), 4.22 (s, 3H), 3.87-3.99 (m, 4H), 3.20-3.28 (m, 2H), 2.68 (s, 3H), 2.44 (br s, 2H). LCMS (ES⁺) Method 2A: m/z 492 (M+H)⁺, RT 2.04 min.

### Example 486: ((1R,5S,6r)-3-(3-(4-chloro-2-methyl-2H-indazol-5-yl)-1H-pyrazolo[3,4-b]pyrazin-6-yl)-6-(thiazol-2-yl)-3-azabicyclo[3.1.0]hexan-6-yl)methanamine (Trifluoro Acetate Salt)

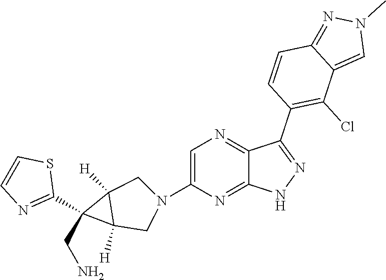 [SMILES: Cn1cc2c(Cl)c(-c3n[nH]c4nc(N5C[C@@H]6[C@H](C5)[C@@]6(CN)c5nccs5)cnc34)ccc2n1]

The above example was synthesized using the procedure reported for Example 2 using Intermediates 44, 151 and 45. Example 486 was obtained as a yellow solid (TFA salt, 5 mg, 41%). ¹H NMR (300 MHz, DMSO-d₆+TFA) δ 8.52 (s, 1H), 8.08 (s, 1H), 8.01 (br s, 3H), 7.77 (d, J=3.3 Hz, 1H), 7.58-7.71 (m, 3H), 4.23 (s, 3H), 3.96-4.11 (m, 4H), 3.35 (br d, J=5.7 Hz, 2H), 2.57-2.52 (m, 2H). LCMS (ES⁺) Method 2A: m/z 478 (M+H)⁺, RT 2.01 min.

### Example 487: ((1R,5S,6r)-3-(3-(4-chloro-2-methyl-2H-indazol-5-yl)-1H-pyrazolo[3,4-b]pyrazin-6-yl)-6-(4-chlorothiazol-2-yl)-3-azabicyclo[3.1.0]hexan-6-yl)methanamine (Trifluoro Acetate Salt)

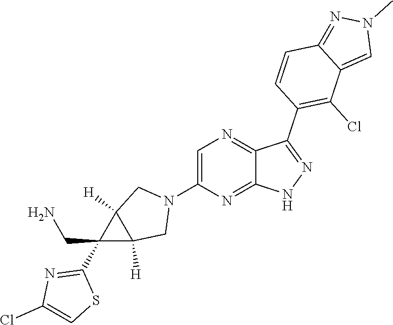 [SMILES: Cn1cc2c(Cl)c(-c3n[nH]c4nc(N5C[C@@H]6[C@H](C5)[C@@]6(CN)c5nc(Cl)cs5)cnc34)ccc2n1]

The above example was synthesized using the procedure reported for Example 2 using Intermediates 44, 125 and 45. Example 487 was obtained as a pale-yellow powder TFA salt (7 mg, 44%). ¹H NMR (300 MHz, DMSO-d₆+TFA) δ 8.53 (s, 1H), 8.08 (s, 1H), 7.99 (br s, 3H), 7.60-7.71 (m, 3H), 4.23 (s, 3H), 3.90-4.08 (m, 4H), 3.30-3.38 (m, 2H), 2.56 (br s, 2H). LCMS (ES⁺) Method 2A: m/z 512 (M+H)⁺, RT 2.15 min.

### Example 488: ((1R,5S,6r)-3-(3-(4-chloro-2-methyl-2H-indazol-5-yl)-1H-pyrazolo[3,4-b]pyrazin-6-yl)-6-(4-(trifluoromethyl)thiazol-2-yl)-3-azabicyclo[3.1.0]hexan-6-yl)methanamine (trifluoro acetate salt)

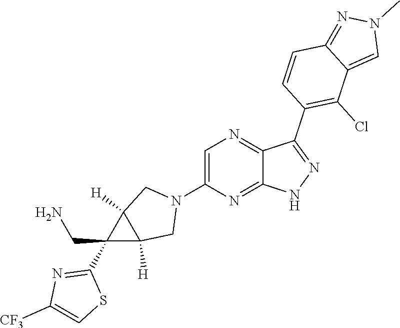 [SMILES: Cn1cc2c(Cl)c(-c3n[nH]c4nc(N5C[C@@H]6[C@H](C5)[C@@]6(CN)c5nc(C(F)(F)F)cs5)cnc34)ccc2n1]

The above example was synthesized using the procedure reported for Example 2 using Intermediates 44, 152 and 45. Example 488 was obtained as light-yellow powder. (TFA salt, 2.4 mg, 45%). ¹H NMR (300 MHz, DMSO-d₆+TFA) δ 8.53 (s, 1H), 8.44 (s, 1H), 8.08 (s, 1H), 8.00 (br s, 3H), 7.59-7.70 (m, 2H), 4.23 (s, 3H), 3.93-4.09 (m, 4H), 3.38 (br d, J=5.9 Hz, 2H), 2.63 (br s, 2H). LCMS (ES⁺) Method 2A: m/z 546 (M+H)⁺, RT 2.28 min.

### Example 489: ((1R,5S,6r)-3-(3-(4-chloro-2-methyl-2H-indazol-5-yl)-1H-pyrazolo[3,4-b]pyrazin-6-yl)-6-(5-fluorothiazol-2-yl)-3-azabicyclo[3.1.0]hexan-6-yl)methanamine (Trifluoro Acetate Salt)

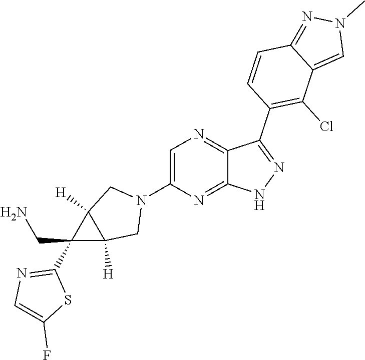 [SMILES: Cn1cc2c(Cl)c(-c3n[nH]c4nc(N5C[C@@H]6[C@H](C5)[C@@]6(CN)c5ncc(F)s5)cnc34)ccc2n1]

The above example was synthesized using the procedure reported for Example 2 using Intermediates 44, 153 and 45. Example 489 was obtained as a pale-yellow powder (TFA salt, 0.5 mg, 22%). LCMS (ES⁺) Method 2A: m/z 496 (M+H)⁺, RT 2.09 min.

### Example 490: ((1R,5S,6r)-3-(3-(4-chloro-2-methyl-2H-indazol-5-yl)-1H-pyrazolo[3,4-b]pyrazin-6-yl)-6-(1,2,5-thiadiazol-3-yl)-3-azabicyclo[3.1.0]hexan-6-yl)methanamine (Trifluoro Acetate Salt)

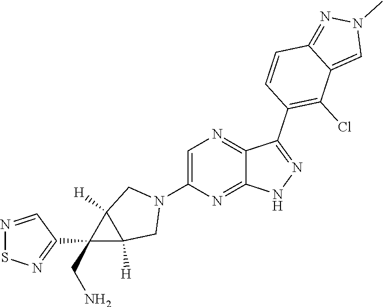 [SMILES: Cn1cc2c(Cl)c(-c3n[nH]c4nc(N5C[C@@H]6[C@H](C5)[C@@]6(CN)c5cnsn5)cnc34)ccc2n1]

The above example was synthesized using the procedure reported for Example 2 using Intermediates 44, 126 and 45. Example 490 was obtained as pale-yellow solid (TFA salt, 2.8 mg, 16%). ¹H NMR (300 MHz, DMSO-d₆) δ 13.41 (s, 1H), 8.87 (s, 1H), 8.57 (s, 1H), 8.10 (s, 1H), 7.89 (br s, 3H), 7.54-7.74 (m, 2H), 4.25 (s, 3H), 4.00 (br s, 4H), 3.41 (s, 2H), 2.73 (br s, 2H). LCMS (ES⁺) Method 2A: m/z 479 (M+H)⁺, RT 1.93 min.

### Example 491: ((1R,5S,6r)-3-(3-(4-chloro-2-methyl-2H-indazol-5-yl)-1H-pyrazolo[3,4-b]pyrazin-6-yl)-6-(pyridin-2-yl)-3-azabicyclo[3.1.0]hexan-6-yl)methanamine (Trifluoro Acetate Salt)

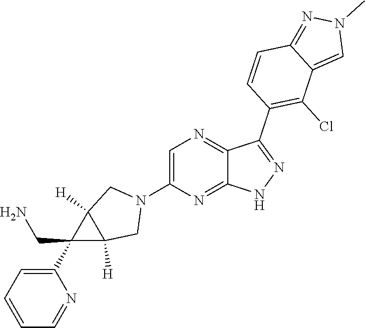 [SMILES: Cn1cc2c(Cl)c(-c3n[nH]c4nc(N5C[C@@H]6[C@H](C5)[C@@]6(CN)c5ccccn5)cnc34)ccc2n1]

The above example was synthesized using the procedure reported for Example 2 using Intermediate 44, Intermediate 129 and Intermediate 45. Example 491 was obtained as yellow powder (TFA salt, 3.0 mg, 12%). ¹H NMR (300 MHz, DMSO-d₆) δ 8.78 (d, J=4.9 Hz, 1H), 8.52 (s, 1H), 8.21-8.29 (m, 1H), 8.12 (s, 1H), 7.90 (br s, 3H), 7.59-7.76 (m, 4H), 4.23 (br s, 3H), 3.99 (br s, 4H), 3.23-3.38 (m, 2H), 2.62 (br s, 2H). LCMS (ES⁺) Method 2A: m/z 472 (M+H)⁺, RT 1.87 min.

### Example 492: ((1R,5S,6r)-3-(3-(7-chloro-2-methylbenzo[d]thiazol-6-yl)-1H-pyrazolo[3,4-b]pyrazin-6-yl)-6-(pyridin-2-yl)-3-azabicyclo[3.1.0]hexan-6-yl)methanamine (Trifluoro Acetate Salt)

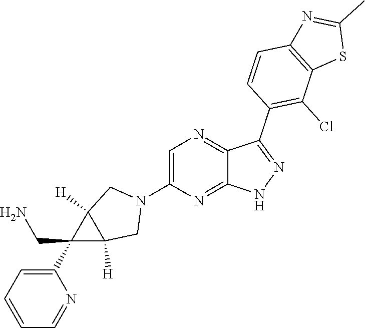 [SMILES: Cc1nc2ccc(-c3n[nH]c4nc(N5C[C@@H]6[C@H](C5)[C@@]6(CN)c5ccccn5)cnc34)c(Cl)c2s1]

The above example was synthesized using the procedure reported for Example 2 using Intermediate 44, Intermediate 129 and Intermediate 54. Example 492 was obtained as a yellow powder (TFA salt; 2.3 mg, 26%). ¹H NMR (300 MHz, DMSO-d₆) δ 8.74 (br d, J=5.8 Hz, 1H), 8.13-8.24 (m, 2H), 7.94-8.06 (m, 2H), 7.85-7.94 (m, 3H), 7.62-7.71 (m, 2H), 3.99 (br s, 4H), 3.24-3.35 (m, 2H), 2.86 (s, 3H), 2.58-2.63 (m, 2H). LCMS (ES⁺) Method 2A: m/z 489 (M+H)⁺, RT 2.17 min.

### Example 493: ((1R,5S,6r)-3-(3-(4-chloro-2-methyl-2H-indazol-5-yl)-1H-pyrazolo[3,4-b]pyrazin-6-yl)-6-(5-methylpyridin-2-yl)-3-azabicyclo[3.1.0]hexan-6-yl)methanamine (Trifluoro Acetate Salt)

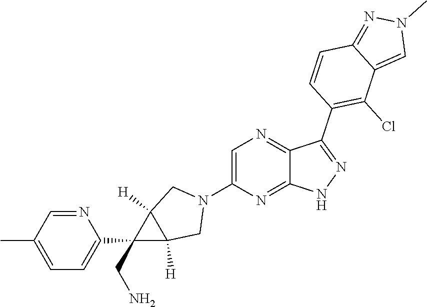

The above example was synthesized using the procedure reported tor Example 2 using Intermediate 44, Intermediate 130 and Intermediate 45. Example 493 was obtained as yellow powder (TFA salt; 3.7 mg, 43%). ¹H NMR (300 MHz, DMSO-d₆+TFA) δ 8.53 (s, 1H), 8.31 (t, J=7.9 Hz, 1H), 8.14 (s, 1H), 7.88 (br s, 3H), 7.78-7.62 (m, 4H), 4.23 (s, 3H), 3.99 (br s, 4H), 3.26 (br d, J=5.0 Hz, 2H), 2.72 (s, 3H), 2.68-2.61 (in, 2H). LCMS (ES⁺) Method 2A: m/z 486 (M+H)⁺, RT 1.88 min.

### Example 494: ((1R,5S,6r)-3-(3-(4-chloro-2-methyl-2H-indazol-5-yl)-1H-pyrazolo[3,4-b]pyrazin-6-yl)-6-(6-chloropyridin-2-yl)-3-azabicyclo[3.1.0]hexan-6-yl)methanamine (Trifluoro Acetate Salt)

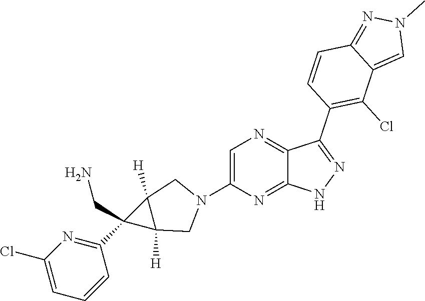 [SMILES: Cn1cc2c(Cl)c(-c3n[nH]c4nc(N5C[C@@H]6[C@H](C5)[C@@]6(CN)c5cccc(Cl)n5)cnc34)ccc2n1]

The above example was synthesized using the procedure reported for Example 2 using Intermediate 44, Intermediate 128 and Intermediate 45; Example 494 was obtained as yellow powder (TFA salt; 5 mg, 32%). ¹H NMR (300 MHz, DMSO-d₆+TFA) δ 8.55 (s, 1H), 8.09 (s, 1H), 7.97-7.77 (m, 4H), 7.74-7.59 (m, 2H), 7.41 (dd, J=7.9, 16.5 Hz, 2H), 4.24 (s, 3H), 4.08-3.88 (m, 4H), 3.43-3.23 (m, 2H), 2.59-2.54 (m, 2H). LCMS (ES⁺) Method 2A: m/z. 506 (M+H)⁺, RT 2.19 min.

### Example 495: ((1R,5S,6r)-3-(3-(4-chloro-2-methyl-2H-indazol-5-yl)-1H-pyrazolo[3,4-b]pyrazin-6-yl)-6-(5-fluoropyridin-2-yl)-3-azabicyclo[3.1.0]hexan-6-yl)methanamine (Trifluoro Acetate Salt)

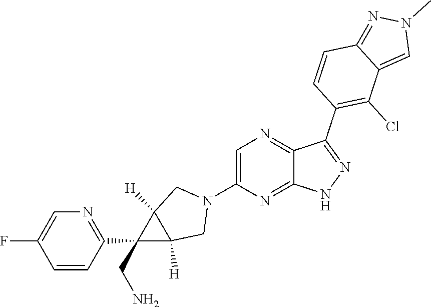 [SMILES: Cn1cc2c(Cl)c(-c3n[nH]c4nc(N5C[C@@H]6[C@H](C5)[C@@]6(CN)c5ccc(F)cn5)cnc34)ccc2n1]

The above example was synthesized using the procedure reported for Example 2 using Intermediate 44, Intermediate 131 and Intermediate 45; Example 495 was obtained as a yellow powder (TFA salt, 6.4 mg, 26%). ¹H NMR (300 MHz, DMSO-d₆) δ 8.49-8.58 (m, 2H), 8.08 (s, 1H), 7.86 (br s, 3H), 7.60-7.82 (m, 3H), 7.38 (dd, J=9.0, 4.1 Hz, 1H), 4.23 (s, 3H), 3.97 (br s, 4H), 3.32 (br d, J=5.4 Hz, 2H) 2.57-2.52 (m, 2H); LCMS (ES⁺) Method 2A: m/z 490 (M+H)⁺, RT 2.08 min.

### Example 496: ((1R,5S,6r)-3-(3-(4-chloro-2-methyl-2H-indazol-5-yl)-1H-pyrazolo[3,4-b]pyrazin-6-yl)-6-(6-methylpyridin-2-yl)-3-azabicyclo[3.1.0]hexan-6-yl)methanamine (Trifluoro Acetate Salt)

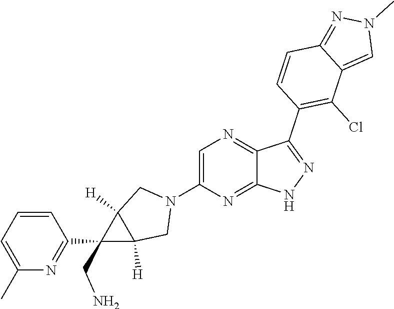 [SMILES: Cc1cccc([C@@]2(CN)[C@@H]3CN(c4cnc5c(-c6ccc7nn(C)cc7c6Cl)n[nH]c5n4)C[C@@H]32)n1]

The above example was synthesized using the procedure reported for Example 2 using Intermediate 44, Intermediate 132 and Intermediate 45; Example 496 was obtained as a yellow powder (TFA salt, 14 mg, 66%). ¹H NMR (300 MHz, DMSO-d₆+TFA) δ 8.53 (s, 1H), 8.31 (t, J=7.9 Hz, 1H), 8.14 (s, 1H), 7.88 (br s, 3H), 7.78-7.62 (m, 4H), 4.23 (s, 3H), 3.99 (br s, 4H), 3.26 (br d, J=5.0 Hz, 2H), 2.72 (s, 3H), 2.68-2.61 (in, 2H). LCMS (ES⁺) Method 2A: m/z 486 (M+H)⁺, RT 1.89 min.

### Example 497: 4-((2-((1R,5S,6r)-6-(aminomethyl)-6-(4-methylthiazol-2-yl)-3-azabicyclo[3.1.0]hexan-3-yl)pyrido[2,3-b]pyrazin-6-yl)thio)-3-chloropyridin-2-amine (Trifluoro Acetate Salt)

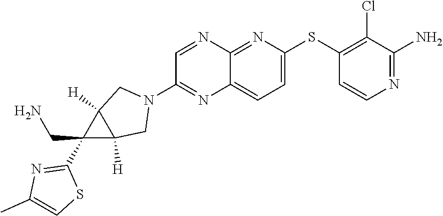 [SMILES: Cc1csc([C@@]2(CN)[C@@H]3CN(c4cnc5nc(Sc6ccnc(N)c6Cl)ccc5n4)C[C@@H]32)n1]

The above compound was synthesized according to the procedure reported for Example 400 using Intermediate 122, 97 and sodium 2-amino-3-chloropyridine-4-thiolate (prepared as reported in the synthesis of Intermediate 42, step 3). Example 497 was obtained as a pale-yellow solid. (TFA salt; 2.4 mg, 48%). ¹H NMR (300 MHz, DMSO-d₆+TFA) δ 8.71 (s, 1H), 8.17 (d, J=8.5 Hz, 1H), 7.95-8.04 (m, 3H), 7.89 (d, J=8.5 Hz, 1H), 7.77 (d, J=6.9 Hz, 1H), 7.21 (s, 1H), 6.49 (d, J=6.9 Hz, 1H), 3.97-4.27 (m, 4H), 3.28-3.41 (m, 2H), 2.57-2.52 (m, 2H), 2.38 (s, 3H); LCMS (ES⁺) Method 2A: m/z 497 (M+H)⁺, RT 1.82 min.

### Example 498: 4-((5-((1R,5S,6r)-6-(aminomethyl)-6-(4-methylthiazol-2-yl)-3-azabicyclo[3.1.0]hexan-3-yl)pyrazin-2-yl)thio)-3-chloropyridin-2-amine (Trifluoro Acetate Salt)

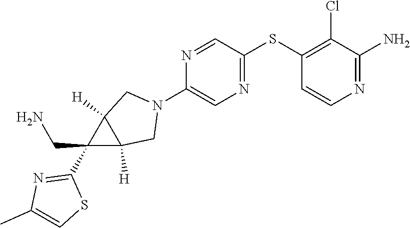 [SMILES: Cc1csc([C@@]2(CN)[C@@H]3CN(c4cnc(Sc5ccnc(N)c5Cl)cn4)C[C@@H]32)n1]

The above compound was synthesized according to the procedure reported for Example 71 using 3-chloro-4-((5-chloropyrazin-2-yl)thio)pyridin-2-amine (prepared as reported in the synthesis of Intermediate 101, step 2-4), Intermediate 70 using DMA and K₂CO₃. The title compound was obtained as a white powder (TFA salt, 5.8 mg, 29%). ¹H NMR (300 MHz, DMSO-d₆+TFA) δ 8.44 (br d, J=1.3 Hz, 1H), 8.17 (br d, J=1.2 Hz, 1H), 8.01 (br s, 3H), 7.76 (d, J=6.8 Hz, 1H), 7.25 (br d, J=1.0 Hz, 1H), 6.15 (d, J=6.9 Hz, 1H), 4.05-3.83 (m, 4H), 3.37-3.20 (m, 2H), 2.62-2.53 (m, 2H), 2.39 (br d, J=0.8 Hz, 3H); LCMS (ES⁺) Method 2: m/z 446 (M+H)⁺, RT 1.73 min.

### Example 499: ((1R,5S,6s)-3-(3-(4-chloro-2-methyl-2H-indazol-5-yl)-1H-pyrazolo[3,4-b]pyrazin-6-yl)-6-(2-fluorophenyl)-3-azabicyclo[3.1.0]hexan-6-yl)methanamine (Trifluoro Acetate Salt)

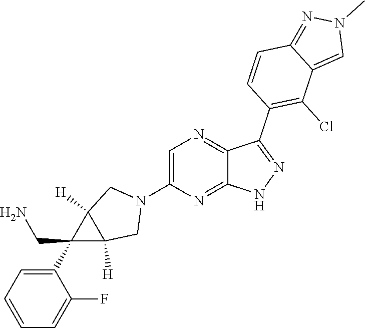 [SMILES: Cn1cc2c(Cl)c(-c3n[nH]c4nc(N5C[C@@H]6[C@H](C5)[C@@]6(CN)c5ccccc5F)cnc34)ccc2n1]

The above example was synthesized using the procedure reported in the Example 2 using Intermediate 44, Intermediate 67 and Intermediate 45; Example 499 was obtained as a pale-yellow powder (TFA salt, 12 mg, 52%). ¹H NMR (300 MHz, DMSO-d₆+TFA) δ 8.55 (s, 1H), 8.12 (s, 1H), 7.75 (br s, 3H), 7.71-7.60 (m, 2H), 7.59-7.49 (m, 1H), 7.48-7.37 (m, 1H), 7.35-7.18 (m, 2H), 4.24 (s, 3H), 4.07-3.86 (m, 4H), 3.13-2.93 (m, 2H), 2.40-2.30 (m, 2H); LCMS (ES⁺) Method 2A: m/z 489 (M+H)⁺, RT 2.25 min.

### Example 501: ((1R,5S,6r)-3-(3-(4-chloro-2-methyl-2H-indazol-5-yl)-1H-pyrazolo[3,4-b]pyrazin-6-yl)-6-(5-methyl-1,3,4-thiadiazol-2-yl)-3-azabicyclo[3.1.0]hexan-6-yl)methanamine (trifluoro acetate salt)

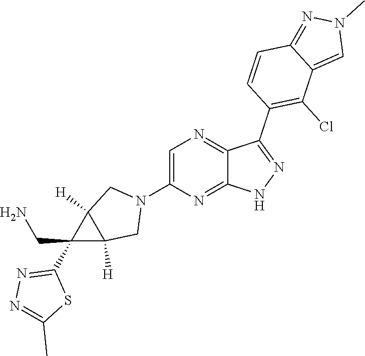 [SMILES: Cc1nnc([C@@]2(CN)[C@@H]3CN(c4cnc5c(-c6ccc7nn(C)cc7c6Cl)n[nH]c5n4)C[C@@H]32)s1]

The above compound was synthesized using the procedure reported for Example 2 using Intermediate 44, Intermediate 146 and Intermediate 45. Example 501 was obtained as an off-white powder (TFA salt, 5.5 mg, 40%). ¹HNMR (300 MHz, DMSO-d₆+TFA) δ 8.55 (s, 1H), 8.09 (s, 1H), 8.03 (br s, 3H), 7.73-7.58 (m, 2H), 4.24 (s, 3H), 4.11-3.89 (m, 4H), 3.45-3.27 (m, 2H), 2.74 (s, 3H), 2.62-2.55 (m, 2H). LCMS (ES⁺) Method 2A: m/z 493 (M+H)⁺, RT 1.89 min.

### Example 502: ((1S,6R,7S)-3-(7-(8-fluoroquinolin-5-yl)-5H-pyrrolo[2,3-b]pyrazin-3-yl)-7-(4-methylthiazol-2-yl)-3-azabicyclo[4.1.0]heptan-7-yl)methanamine (Trifluoro Acetate Salt)

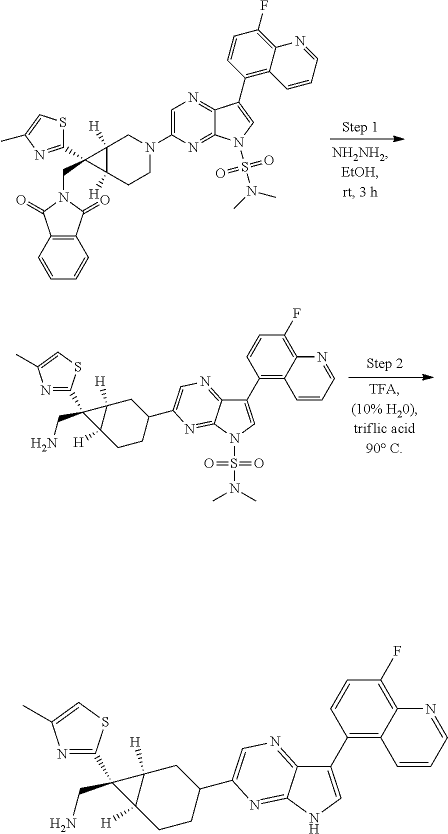 [SMILES: Cc1csc([C@@]2(CN3C(=O)c4ccccc4C3=O)[C@@H]3CCN(c4cnc5c(-c6ccc(F)c7ncccc67)cn(S(=O)(=O)N(C)C)c5n4)C[C@@H]32)n1 ; Cc1csc([C@@]2(CN)[C@@H]3CCC(c4cnc5c(-c6ccc(F)c7ncccc67)cn(S(=O)(=O)N(C)C)c5n4)C[C@@H]32)n1 ; Cc1csc([C@@]2(CN)[C@@H]3CCC(c4cnc5c(-c6ccc(F)c7ncccc67)c[nH]c5n4)C[C@@H]32)n1]

### Step 1: 3-((1S,6R,7S)-7-(aminomethyl)-7-(4-methylthiazol-2-yl)-3-azabicyclo[4.1.0]heptan-3-yl)-7-(8-fluoroquinolin-5-yl)-N,N-dimethyl-5H-pyrrolo[2,3-b]pyrazine-5-sulfonamide

To a solution of 3-((1S,6R,7S)-7-((1,3-dioxoisoindolin-2-yl)methyl)-7-(4-methylthiazol-2-yl)-3-azabicyclo[4.1.0]heptan-3-yl)-7-(8-fluoroquinolin-5-yl)-N,N-dimethyl-5H-pyrrolo[2,3-b]pyrazine-5-sulfonamide (prepared using the procedure reported of Example 2 (step 1-2, with DMSO at 120° C. using Intermediates 149 and Intermediate 40, and then 8-fluoro-5-(4,4,5,5-tetramethyl-1,3,2-dioxaborolan-2-yl)quinoline for the last step; 12 mg, 0.02 mmol) in EtOH (1 mL) N₂H₄·H₂O (0.02 mL, 0.5 mmol) was added. The resulting mixture was stirred at rt for 3 h. Volatiles were removed under reduced pressure and the resulting crude compound (9.8 mg) was used in the next step without further purification. LCMS (ES⁺) Method 2A: m/z 593 (M+H)⁺, RT 1.56 min.

### Step 2: ((1S,6R,7S)-3-(7-(8-fluoroquinolin-5-yl)-5H-pyrrolo[2,3-b]pyrazin-3-yl)-7-(4-methylthiazol-2-yl)-3-azabicyclo[4.1.0]heptan-7-yl)methanamine

3-((1S,6R,7S)-7-(aminomethyl)-7-(4-methylthiazol-2-yl)-3-azabicyclo[4.1.0]heptan-3-yl)-7-(8-fluoroquinolin-5-yl)-N,N-dimethyl-5H-pyrrolo[2,3-b]pyrazine-5-sulfonamide (9.8 mg, 0.02 mmol) was treated with TFA/triflic acid (1:1; 1 mL) and H₂O (0.05 mL). The resulting mixture was heated under MW irradiation at 90° C. for 30 min. The reaction was purified by preparative HPLC (eluting with 0-40% H₂O/MeCN+0.1% TFA) to afford the title compound as yellow solid (TFA salt, 2 mg, 25%). ¹H NMR (300 MHz, DMSO-d₆+TFA) δ 12.00 (s, 1H), 9.07 (d, J=3.4 Hz, 1H), 8.65 (d, J=9.9 Hz, 1H), 8.21 (s, 1H), 7.94 (br s, 3H), 7.68-7.84 (m, 4H), 7.16 (s, 1H), 4.21-4.35 (m, 2H), 3.56-3.68 (m, 2H), 3.39-3.53 (m, 1H), 2.81-2.93 (m, 1H), 2.34-2.41 (m, 4H), 1.97-2.10 (m, 1H), 1.77-1.92 (m, 2H); LCMS (ES⁺) Method 2A: m/z 486 (M+H)⁺, RT 2.14 min.

### Example 507: ((1S,6R,7R)-7-(2-fluorophenyl)-3-(7-(8-fluoroquinolin-5-yl)-5H-pyrrolo[2,3-b]pyrazin-3-yl)-3-azabicyclo[4.1.0]heptan-7-yl)methanamine (Trifluoro Acetate Salt)

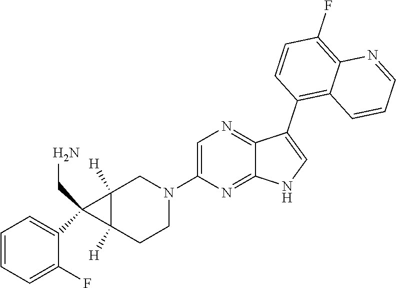 [SMILES: NC[C@]1(c2ccccc2F)[C@@H]2CCN(c3cnc4c(-c5ccc(F)c6ncccc56)c[nH]c4n3)C[C@@H]21]

The above example was synthesized using the procedure reported in the Example 2 (step 1-2 with DMSO at 120° C.) using Intermediate 149 and Intermediate 51, and 8-fluoro-5-(4,4,5,5-tetramethyl-1,3,2-dioxaborolan-2-yl)quinoline) in the last step. The final deprotection step was performed as described for Example 156 (step 4). The title compound was obtained as an orange solid (TFA salt, 7 mg, 59%). ¹H NMR (300 MHz, DMSO-d₆+TFA) δ 12.07 (br s, 1H), 9.11 (dd, J=4.5, 1.3 Hz, 1H), 8.79-8.72 (m, 1H), 8.23 (s, 1H), 7.89-7.64 (m, 7H), 7.53-7.46 (m, 1H), 7.40-7.31 (m, 1H), 7.23-7.14 (m, 2H), 4.42-4.20 (m, 2H), 3.60 (dd, J=14.0, 6.2 Hz, 1H), 3.22 (br d, J=5.2 Hz, 2H), 2.91-2.79 (m, 1H), 2.42-2.31 (m, 1H), 1.91-1.75 (m, 2H), 1.60-1.50 (m, 1H); LCMS (ES⁺) Method 2A: m/z 483 (M+H)⁺, RT 2.27 min.

### Example 508: 5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinoline-8-sulfonyl fluoride (Trifluoro Acetate Salt)

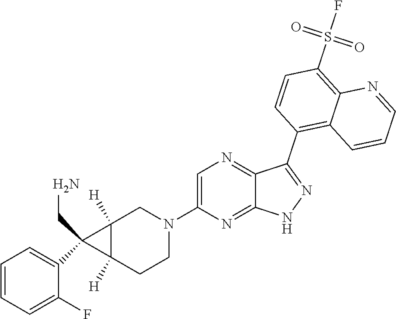 [SMILES: NC[C@]1(c2ccccc2F)[C@@H]2CCN(c3cnc4c(-c5ccc(S(=O)(=O)F)c6ncccc56)n[nH]c4n3)C[C@@H]21]

The above example was synthesized according to the procedure described in Example 77 (step 2) using Intermediate 116 and 5-(4,4,5,5-tetramethyl-1,3,2-dioxaborolan-2-yl)quinoline-8-sulfonyl fluoride (prepared as reported in the synthesis of Intermediate 15 starting from Intermediate 59) and Example 91 (step 4) for the final deprotection step. The title compound was obtained as yellow powder (TFA salt, 7 mg, 37%). ¹H NMR (300 MHz, DMSO-d₆+TFA) δ 9.61 (dd, J 1.7, 8.7 Hz, 1H), 9.22 (dd, J=1.6, 4.2 Hz, 1H), 8.81-8.68 (m, 2H), 8.57 (s, 1H), 7.88 (dd, J=4.2, 8.8 Hz, 1H), 7.68 (br s, 3H), 7.54-7.44 (m, 1H), 7.43-7.31 (m, 1H), 7.27-7.14 (m, 2H), 4.49 (d, J=14.0 Hz, 1H), 4.44-4.30 (m, 1H), 3.79 (dd, J=6.3, 14.3 Hz, 1H), 3.24-2.89 (m, 3H), 2.43-2.30 (m, 1H), 1.91-1.71 (m, 2H), 1.63-1.51 (m, 1H); LCMS (ES⁺) Method 2A: m/z 548 (M+H)⁺, RT 2.84 min.

### Example 510: ((1R,5S,6s)-3-(3-(8-fluoroquinolin-5-yl)-1H-pyrazolo[3,4-b]pyrazin-6-yl)-6-phenyl-3-azabicyclo[3.1.0]hexan-6-yl)methanamine (Trifluoro Acetate Salt)

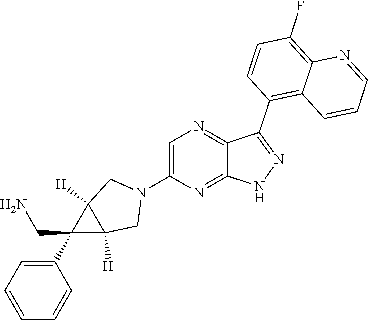 [SMILES: NC[C@]1(c2ccccc2)[C@@H]2CN(c3cnc4c(-c5ccc(F)c6ncccc56)n[nH]c4n3)C[C@@H]21]

The above example was synthesized using the procedure reported in the Example 2 using Intermediate 44, Intermediate 121 (step 1) and 8-fluoro-5-(4,4,5,5-tetramethyl-1,3,2-dioxaborolan-2-yl)quinoline (step 2). Example 510 was obtained as a pale-yellow powder (TFA salt, 4 mg, 66%). ¹H NMR (300 MHz, DMSO-d₆+TFA) δ 9.54 (d, J=8.8 Hz, 1H), 9.11 (dd, J=4.4, 1.5 Hz, 1H), 8.49 (dd, J=8.3, 5.1 Hz, 1H), 8.16 (s, 1H), 7.92-7.65 (m, 5H), 7.54-7.27 (m, 5H), 3.96 (br s, 4H), 3.15-3.02 (m, 2H), 2.39-2.32 (m, 2H); LCMS (ES⁺) Method 2: m/z 452 (M+H)⁺, RT 2.12 min.

### Example 511: ((1R,5S,6r)-3-(3-(5-chloroquinoxalin-6-yl)-1H-pyrazolo[3,4-b]pyrazin-6-yl)-6-(1-methyl-1H-pyrazol-3-yl)-3-azabicyclo[3.1.0]hexan-6-yl)methanamine (Trifluoro Acetate Salt)

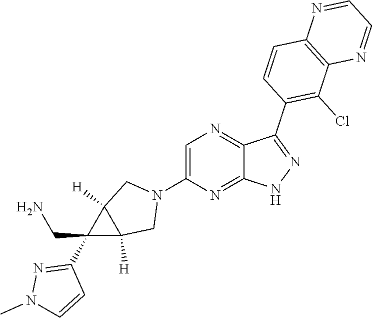 [SMILES: Cn1ccc([C@@]2(CN)[C@@H]3CN(c4cnc5c(-c6ccc7nccnc7c6Cl)n[nH]c5n4)C[C@@H]32)n1]

The above compound was synthesized using the procedure reported for Example 2 using Intermediate 44, Intermediate 127 (step 1) and Intermediate 82 (step 2). Example 511 was obtained as a yellow powder (TFA salt, 8 mg, 90%). ¹H NMR (300 MHz, DMSO-d₆+TFA) δ 9.12 (dd, J=1.8, 11.4 Hz, 2H), 8.27-8.17 (m, 2H), 8.12 (s, 1H), 7.84 (br s, 3H), 7.69 (d, J=2.1 Hz, 1H), 6.13 (d, J=2.3 Hz, 1H), 4.03-3.88 (m, 4H), 3.83 (s, 3H), 3.28-3.10 (m, 2H), 2.36-2.29 (m, 2H); LCMS (ES⁺) Method 2A: m/z 473 (M+H)⁺, RT 1.94 min.

### Example 514 ((1R,5S,6r)-3-(3-(4-chloro-2-methyl-2H-indazol-5-yl)-1H-pyrazolo[3,4-b]pyrazin-6-yl)-6-(2-methyloxazol-4-yl)-3-azabicyclo[3.1.0]hexan-6-yl)methanamine (Trifluoro Acetate Salt)

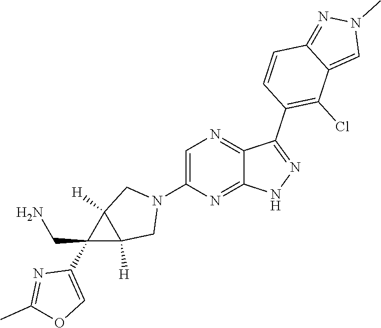

The above compound was synthesized using the procedure reported for Example 2 using Intermediate 44, 65 and 45; Example 514 was obtained as a white powder (TFA salt, 1.9 mg, 22%). ¹H NMR (300 MHz, DMSO-d₆) δ 13.38 (s, 1H), 8.56 (s, 1H), 8.08 (s, 1H), 7.95 (s, 1H), 7.83 (br s, 3H), 7.66 (q, J=9.3 Hz, 2H), 4.24 (s, 3H), 4.04-3.75 (m, 4H), 3.20-3.05 (m, 2H), 2.42 (s, 3H), 2.39-2.30 (m, 2H); LCMS (ES⁺) Method 2A: m/z 476 (M+H)⁺, RT 1.94 min.

### Example 515: ((1R,5S,6r)-3-(6-((3-chloro-2-(1H-pyrazol-1-yl)pyridin-4-yl)thio)pyrido[2,3-b]pyrazin-2-yl)-6-(4-methylthiazol-2-yl)-3-azabicyclo[3.1.0]hexan-6-yl)methanamine (trifluoro acetate salt)

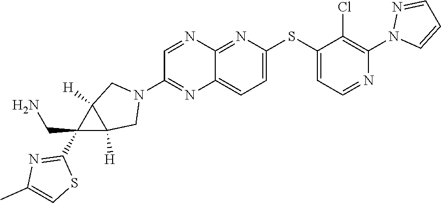 [SMILES: Cc1csc([C@@]2(CN)[C@@H]3CN(c4cnc5nc(Sc6ccnc(-n7cccn7)c6Cl)ccc5n4)C[C@@H]32)n1]

The above example was synthesized according to the procedure described in Example 395 using Intermediate 97, 100 and Intermediate 122 and Example 1 step 6 for the final deprotection. Example 515 was obtained as a yellow powder (TFA salt, 7 mg, 37%). ¹H NMR (300 MHz, DMSO-d₆+TFA) δ 8.67 (s, 1H), 8.34 (d, J=5.1 Hz, 1H), 8.26 (d, J=2.5 Hz, 1H), 8.14 (d, J=8.5 Hz, 1H), 7.97 (br s, 3H), 7.85 (d, J=8.5 Hz, 1H), 7.81 (d, J=1.2 Hz, 1H), 7.35 (d, J=5.2 Hz, 1H), 7.23-7.19 (m, 1H), 6.60-6.52 (m, 1H), 4.27-3.95 (m, 4H), 3.33 (br s, 2H), 2.55-2.52 (m, 2H), 2.38 (d, J=0.7 Hz, 3H); LCMS (ES⁺) Method 2A: m/z 548 (M+H)⁺, RT 2.46 min.

### Example 516 ((1R,5S,6r)-3-(3-(3-chloro-2-(1H-pyrazol-1-yl)pyridin-4-yl)-1H-pyrazolo[3,4-b]pyrazin-6-yl)-6-(4-methylthiazol-2-yl)-3-azabicyclo[3.1.0]hexan-6-yl)methanamine (trifluoro acetate salt)

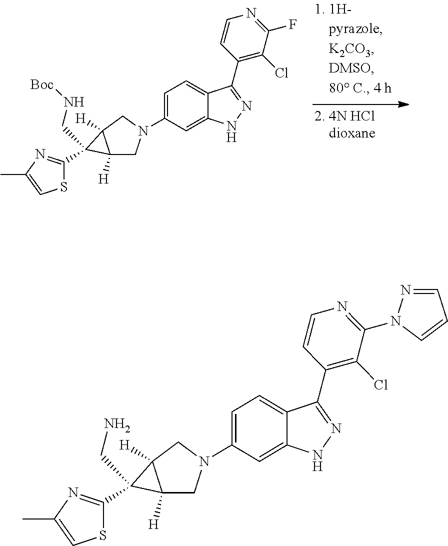 [SMILES: Cc1csc([C@@]2(CNC(=O)OC(C)(C)C)[C@@H]3CN(c4ccc5c(-c6ccnc(F)c6Cl)n[nH]c5c4)C[C@@H]32)n1 ; Cc1csc([C@@]2(CN)[C@@H]3CN(c4ccc5c(-c6ccnc(-n7cccn7)c6Cl)n[nH]c5c4)C[C@@H]32)n1]

### Step 1: tert-butyl (((1R,5S,6r)-3-(3-(3-chloro-2-(1H-pyrazol-1-yl)pyridin-4-yl)-1H-pyrazolo[3,4-b]pyrazin-6-yl)-6-(4-methylthiazol-2-yl)-3-azabicyclo[3.1.0]hexan-6-yl)methyl)carbamate

A mixture of tert-butyl (((1R,5S,6r)-3-(3-(3-chloro-2-fluoropyridin-4-yl)-1H-pyrazolo[3,4-b]pyrazin-6-yl)-6-(4-methylthiazol-2-yl)-3-azabicyclo[3.1.0]hexan-6-yl)methyl)carbamate (synthesized using the procedure reported for Example 77 step 2 using Intermediate 123 and (3-chloro-2-fluoropyridin-4-yl)boronic acid) (12 mg, 0.02 mmol), 1H-pyrazole (2.6 mg, 0.037 mmol) and K₂CO₃ (5.2 mg, 0.037 mmol) in DMSO (0.5 ml) was stirred at 80° C. for 4 h. Then the mixture was diluted with EtOAc and washed with H₂O (×4). The organic layer was dried over Na₂SO₄, filtered and concentrated, and the resulting crude residue (14 mg) was used in the next step without further purification. LCMS (ES⁺) Method 1A: m/z 689 (M+H)⁺, RT 2.25 min

### Step 2: ((1R,5S,6r)-3-(3-(3-chloro-2-(1H-pyrazol-1-yl)pyridin-4-yl)-1H-pyrazolo[3,4-b]pyrazin-6-yl)-6-(4-methylthiazol-2-yl)-3-azabicyclo[3.1.0]hexan-6-yl)methanamine

The title compound was prepared according to the procedure descripted in the step 4 of Example 91. Example 516 was obtained as an off-white solid (TFA salt; 4.5 mg, 38% over 2 steps). ¹H NMR (300 MHz, DMSO-d₆+TFA) δ 8.63 (d, J=5.0 Hz, 1H), 8.28 (d, J=2.5 Hz, 1H), 8.15 (s, 1H), 8.08-7.88 (m, 4H), 7.82 (d, J=1.2 Hz, 1H), 7.24 (br d, J=1.0 Hz, 1H), 6.56 (dd, J=1.8, 2.4 Hz, 1H), 4.16-3.81 (m, 4H), 3.40-3.20 (m, 2H), 2.58-2.53 (m, 2H), 2.38 (br d, J=0.8 Hz, 3H); LCMS (ES⁺) Method 2A: m/z 505 (M+H)⁺, RT 2.14

### Example 520: ((1R,5S,6r)-3-(5-((3-chloro-2-(1H-pyrazol-1-yl)pyridin-4-yl)thio)pyrazin-2-yl)-6-(4-methylthiazol-2-yl)-3-azabicyclo[3.1.0]hexan-6-yl)methanamine (Trifluoro Acetate Salt)

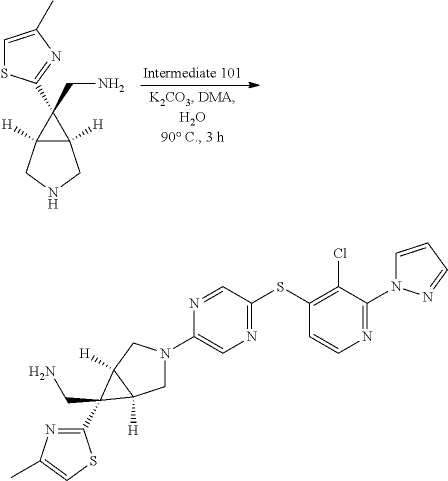 [SMILES: Cc1csc([C@@]2(CN)[C@@H]3CNC[C@@H]32)n1 ; Cc1csc([C@@]2(CN)[C@@H]3CN(c4cnc(Sc5ccnc(-n6cccn6)c5Cl)cn4)C[C@@H]32)n1]

A suspension of Intermediate 70 (10 mg, 0.05 mmol), Intermediate 101 (20 mg, 0.06 mmol) and K₂CO₃ (33 mg, 0.24 mmol) in DMA (0.3 mL) and H₂O (0.2 mL) was heated at 90° C. for 3 h. The reaction was directly purified by flash chromatography (C18, 0-30% H₂O/CH₃CN+0.1% TFA) to obtain the title compound that was purified again by normal phase flash chromatography (0-10% MeOH in EtOAc). Fractions containing product were collected and volatiles removed under reduced pressure. The residue was dissolved in MeCN and H₂O, TFA (0.1 mL) was added and after lyophilization the title compound was obtained as a greenish solid (TFA salt, 5.86 mg, 20%). ¹H NMR (300 MHz, DMSO-d₆+TFA) δ 8.45 (br d, J=1.3 Hz, 1H), 8.29-8.15 (m, 3H), 8.01 (br s, 3H), 7.83-7.78 (m, 1H), 7.23 (br d, J=1.0 Hz, 1H), 6.78 (d, J=5.3 Hz, 1H), 6.58-6.50 (m, 1H), 4.08-3.82 (m, 4H), 3.41-3.22 (m, 2H), 2.63-2.53 (m, 2H), 2.38 (br d, J=0.9 Hz, 3H); LCMS (ES⁺) Method 2A: m/z 497 (M+H)⁺, RT 2.42 min.

### Example 522: ((1S,6R,7R)-3-(3-(3-chloro-2-(1H-pyrrol-1-yl)pyridin-4-yl)-1H-pyrazolo[3,4-b]pyrazin-6-yl)-7-(2-fluorophenyl)-3-azabicyclo[4.1.0]heptan-7-yl)methanamine (trifluoro acetate salt)

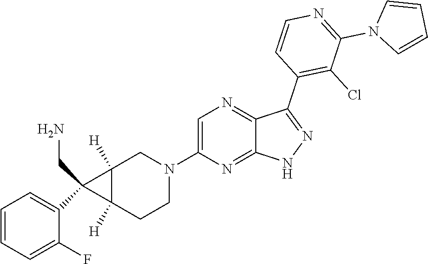 [SMILES: NC[C@]1(c2ccccc2F)[C@@H]2CCN(c3cnc4c(-c5ccnc(-n6cccc6)c5Cl)n[nH]c4n3)C[C@@H]21]

The above compound was synthesized using the procedure reported for Example 148 using Intermediate 116 and 3-chloro-4-iodo-2-(1H-pyrrol-1-yl)pyridine (prepared as described for Example 451 using 1H-Pyrrole and NaH (60% in mineral oil)). The final deprotection step was performed as described for Example 91 (step 4) and the title compound obtained as a TFA salt (1 mg, 4% over 3 steps). LCMS (ES⁺) Method 2: m/z 515 (M+H)⁺, RT 2.73 min.

### Example 523: 6-((1S,6R,7R)-7-(aminomethyl)-7-(2-fluorophenyl)-3-azabicyclo[4.1.0]heptan-3-yl)-3-((5-chloroquinoxalin-6-yl)thio)pyrazin-2-amine (Trifluoro Acetate Salt)

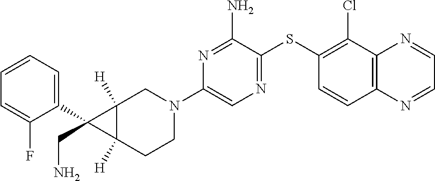 [SMILES: NC[C@]1(c2ccccc2F)[C@@H]2CCN(c3cnc(Sc4ccc5nccnc5c4Cl)c(N)n3)C[C@@H]21]

The above example was synthesized using the procedure reported for Example 384 using Intermediate 51 and 6-chloro-3-((5-chloroquinoxalin-6-yl)thio)pyrazin-2-amine (prepared as reported for Intermediate 41, using NaOMe in MeOH in step 2, starting from 6-bromo-5-chloroquinoxaline). The title compound was obtained as pale-yellow solid (TFA salt; 2 mg, 3.5%). ¹H NMR (400 MHz, DMSO-d₆) δ 9.02 (d, J=1.8 Hz, 1H), 8.95 (d, J=1.8 Hz, 1H), 7.96 (d, J=9.2 Hz, 1H), 7.78-7.60 (m, 4H), 7.52-7.43 (m, 1H), 7.42-7.32 (m, 1H), 7.28-7.16 (m, 2H), 7.10 (d, J=9.2 Hz, 1H), 6.32 (br s, 2H), 4.36-4.23 (m, 2H), 3.70-3.60 (m, 1H), 3.23-3.04 (m, 2H), 2.85-2.72 (m, 1H), 2.35-2.24 (m, 1H), 1.85-1.65 (m, 2H), 1.54-1.45 (m, 1H). LCMS (ES⁺) Method 2: m/z 508-510 (M+H)⁺, RT 1.17 min.

### Example 524: 6-((1S,6R,7R)-7-(aminomethyl)-7-(2-fluorophenyl)-3-azabicyclo[4.1.0]heptan-3-yl)-3-(quinolin-4-ylthio)pyrazin-2-amine (Trifluoro Acetate Salt)

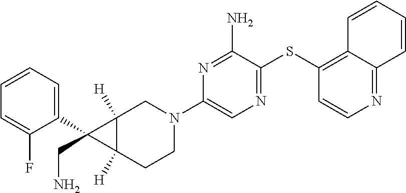 [SMILES: NC[C@]1(c2ccccc2F)[C@@H]2CCN(c3cnc(Sc4ccnc5ccccc45)c(N)n3)C[C@@H]21]

The above example was synthesized using the procedure reported for Example 384 using Intermediate 51 and 6-chloro-3-(quinolin-4-ylthio)pyrazin-2-amine (prepared as reported for Intermediate 41, using NaOMe in MeOH in step 2, starting from 4-bromoquinoline). The title compound was obtained as a yellow solid (TFA salt; 0.4 mg, 1.5%). ¹H NMR (400 MHz, DMSO-d₆) δ 8.63 (d, J=5.0 Hz, 1H), 8.19 (d, J=8.3 Hz, 1H), 8.04 (d, J=8.1 Hz, 1H), 7.85 (br t, J=7.1 Hz, 1H), 7.69 (s, 5H), 7.51-7.44 (m, 1H), 7.42-7.33 (m, 1H), 7.27-7.16 (m, 2H), 6.77 (d, J=4.8 Hz, 1H), 6.33 (br s, 2H), 4.30 (br d, J=13.8 Hz, 2H), 3.69-3.59 (m, 1H), 3.19-3.05 (m, 2H), 2.82-2.73 (m, 1H), 2.33-2.26 (m, 1H), 1.85-1.63 (m, 2H), 1.53-1.45 (m, 1H). LCMS (ES⁺) Method 2: m/z 473 (M+H)⁺, RT 0.95 min.

### Example 525: 5-((3-amino-5-((1S,6R,7R)-7-(aminomethyl)-7-(2-fluorophenyl)-3-azabicyclo[4.1.0]heptan-3-yl)pyrazin-2-yl)thio)quinoline-8-carboxamide (Trifluoro Acetate Salt)

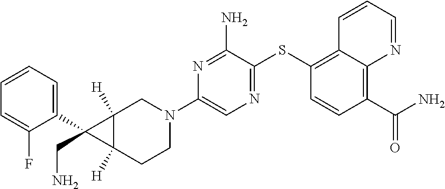 [SMILES: NC[C@]1(c2ccccc2F)[C@@H]2CCN(c3cnc(Sc4ccc(C(N)=O)c5ncccc45)c(N)n3)C[C@@H]21]

The above example was synthesized using the procedure reported for Example 384 using Intermediate 51 and 5-((3-amino-5-chloropyrazin-2-yl)thio)quinoline-8-carboxamide (prepared as reported for Intermediate 41, using NaOMe in MeOH in step 2, starting from 5-bromoquinoline-8-carboxamide). The title compound was obtained as a yellow solid (TFA salt; 11 mg, 5%). ¹H NMR (400 MHz, DMSO-d₆) δ 10.08 (br d, J=3.9 Hz, 1H), 9.09 (dd, J=1.8, 4.4 Hz, 1H), 8.83 (dd, J=1.8, 8.6 Hz, 1H), 8.40 (d, J=7.9 Hz, 1H), 7.90-7.83 (m, 1H), 7.77-7.72 (m, 1H), 7.72-7.66 (m, 3H), 7.62 (s, 1H), 7.51-7.43 (m, 1H), 7.42-7.34 (m, 1H), 7.25-7.15 (m, 3H), 6.46-6.11 (m, 2H), 4.41-4.16 (m, 2H), 3.68-3.51 (m, 1H), 3.25-3.01 (m, 2H), 2.84-2.69 (m, 1H), 2.33-2.22 (m, 1H), 1.82-1.65 (m, 2H), 1.55-1.38 (m, 1H). LCMS (ES⁺) Method 2: m/z 516 (M+H)⁺, RT 1.10 min.

### Example 526: 6-((1R,5S,6r)-6-(aminomethyl)-6-(4-methylthiazol-2-yl)-3-azabicyclo[3.1.0]hexan-3-yl)-5-chloro-3-(2,3-dichlorophenyl)-2-methylpyrimidin-4(3H)-one (Trifluoro Acetate Salt)

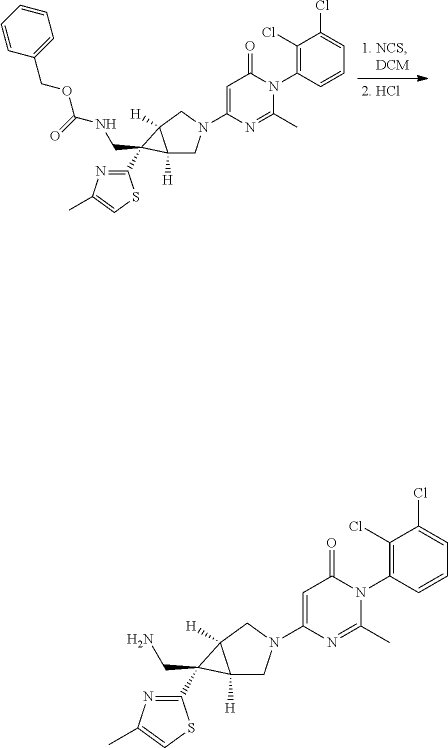 [SMILES: Cc1csc([C@@]2(CNC(=O)OCc3ccccc3)[C@@H]3CN(c4cc(=O)n(-c5cccc(Cl)c5Cl)c(C)n4)C[C@@H]32)n1 ; Cc1csc([C@@]2(CN)[C@@H]3CN(c4cc(=O)n(-c5cccc(Cl)c5Cl)c(C)n4)C[C@@H]32)n1]

NCS (11 mg, 0.08 mmol) was added to a solution of benzyl (((1R,5S,6r)-3-(1-(2,3-dichlorophenyl)-2-methyl-6-oxo-1,6-dihydropyrimidin-4-yl)-6-(4-methylthiazol-2-yl)-3-azabicyclo[3.1.0]hexan-6-yl)methyl)carbamate (synthesized as reported for Example 411 using Intermediate 111 and 124; 42 mg, 0.07 mmol) in DCM (0.7 mL) at rt. After 30 min the mixture was diluted with DCM and washed with H₂O. The organic phase was dried over Na₂SO₄, filtered and the solvent removed under reduced pressure to afford a brown solid (45 mg) that was used in the next step without any further purification. LCMS (ES⁺) Method 1: m/z 630 (M+H)⁺, RT 2.25 min. The crude residue was used according to the procedure reported for Example 77 (step 3) and obtained as a white powder (TFA salt; 19 mg, 45% over 2 steps). ¹H NMR (300 MHz, DMSO-d₆) δ 8.05 (br s, 3H), 7.86-7.77 (m, 1H), 7.56 (d, J=4.9 Hz, 2H), 7.20 (s, 1H), 4.39-4.24 (m, 2H), 4.24-4.12 (m, 2H), 3.36 (br d, J=5.6 Hz, 2H), 2.43-2.33 (m, 5H), 2.01 (s, 3H). LCMS (ES⁺) Method 2: m/z 496 (M+H)⁺, RT 2.62 min.

### Example 527: ((1S,6R,7R)-7-(2,5-difluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine (Trifluoro Acetate Salt)

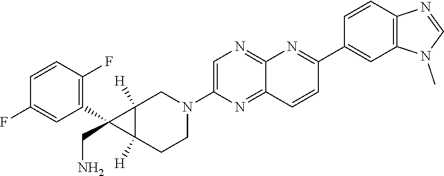 [SMILES: Cn1cnc2ccc(-c3ccc4nc(N5CC[C@@H]6[C@H](C5)[C@@]6(CN)c5cc(F)ccc5F)cnc4n3)cc21]

The above example was synthesized using the procedure reported for Example 394 using Intermediate 97, 102 and 1-methyl-6-(4,4,5,5-tetramethyl-1,3,2-dioxaborolan-2-yl)-1H-benzo[d]imidazole; Example 527 was obtained as yellow solid (3 mg, 12%). LCMS (ES⁺) Method 2: m/z 498 (M+H)⁺, RT 1.16 min.

### Example 529: ((1S,6R,7R)-7-(2,5-difluorophenyl)-3-(6-(3-fluoro-2-methylpyridin-4-yl)pyrido[2,3-b]pyrazin-2-yl)-3-azabicyclo[4.1.0]heptan-7-yl)methanamine (Trifluoro Acetate Salt)

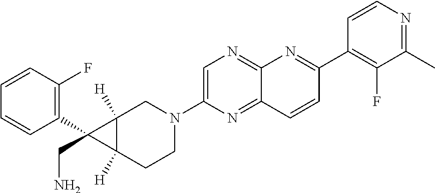 [SMILES: Cc1nccc(-c2ccc3nc(N4CC[C@@H]5[C@H](C4)[C@@]5(CN)c4ccccc4F)cnc3n2)c1F]

The above example was synthesized using the procedure reported for Example 394 using Intermediate 51, 97 and 3-fluoro-2-methyl-4-(4,4,5,5-tetramethyl-1,3,2-dioxaborolan-2-yl)pyridine; Example 529 was obtained as yellow solid (4 mg, 18%). LCMS (ES⁺) Method 2: m/z 459 (M+H)⁺, RT 1.25 min.

### Example 530: ((1S,6R,7R)-3-(6-(2-(1H-pyrazol-1-yl)pyridin-4-yl)pyrido[2,3-b]pyrazin-2-yl)-7-(2-fluorophenyl)-3-azabicyclo[4.1.0]heptan-7-yl)methanamine (Trifluoro Acetate Salt)

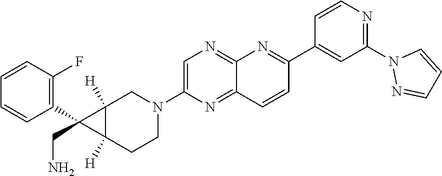 [SMILES: NC[C@]1(c2ccccc2F)[C@@H]2CCN(c3cnc4nc(-c5ccnc(-n6cccn6)c5)ccc4n3)C[C@@H]21]

The above example was synthesize using the procedure reported or Example 394 using Intermediate 51, 97 and 2-fluoropyridin-4-yl)boronic acid, followed by 1H-pyrazole and K₂CO₃ in DMSO as described in Example 516 step 1; Example 530 was obtained as yellow solid (1.8 mg, 21%). LCMS (ES⁺) Method 2: m/z 493 (M+H)⁺, RT 1.45 min.

### Example 531: 6-(2-((1S,6R,7R)-7-(aminomethyl)-7-(2,5-difluorophenyl)-3-azabicyclo[4.1.0]heptan-3-yl)pyrido[2,3-b]pyrazin-6-yl)-3,4-dihydroquinolin-2(1H)-one (Trifluoro Acetate Salt)

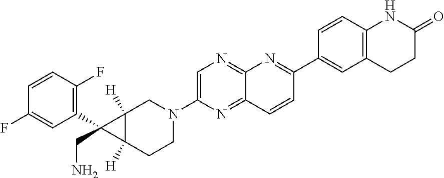 [SMILES: NC[C@]1(c2cc(F)ccc2F)[C@@H]2CCN(c3cnc4nc(-c5ccc6c(c5)CCC(=O)N6)ccc4n3)C[C@@H]21]

The above example was synthesized using the procedure reported for Example 394 using Intermediate 97, 102 and 5-(4,4,5,5-tetramethyl-1,3,2-dioxaborolan-2-yl)-3,4-dihydroquinolin-2(1H)-one; Example 531 was obtained as yellow solid (2.9 mg, 32%). LCMS (ES⁺) Method 2: m/z 502 (M+H)⁺, RT 1.09 min.

### BIOLOGY

### SHP2 Inhibition Enzymatic Assay

The assay was performed as described in YP Chen et al, Nature (535)₂₀₁₆. Assay volume of 20 μL/well was assembled in 384 well black polystyrene low-binding microplates (Greiner), using the following buffer: 60 mM HEPES pH 7.2, 75 mM NaCl, 75 mM KCl, 1 mM EDTA pH 8, 0.05% tween-20, 5 mM DTT. The SHP-2 enzyme (synthetized by Origene, Met1-Leu525, cat #TP750155) was used at a final concentration of 0.5 nM. The enzyme was activated by 500 nM IRS1 peptide (sequence: H2N-LN(pY)IDLDLV(dPEG8)LST(pY)ASINFQK-amide SEQ ID No. 1) and incubated with 75 μM DiFMUP (Sigma) as substrate.

Briefly, DMSO serially diluted testing compounds were transferred to the bottom of the assay plate. SHP2 was then added together with the IRS1 peptide. 30 min post incubation, the DiFMUP substrate was added to the reaction and incubated 30 min at room temperature. Finally 5 μL of 160 μM bpV (Potassium bisperoxo[1,10-phenanthroline]oxovanadate [V], Sigma) were added to stop and quench the reaction. The fluorescence was detected by a microplate reader (Envision, PerkinElmer) according to the DiFMUP excitation and emission wavelength. The lower the fluorescence the higher the SHP2 inhibition. The activity of each compound dilution was calculated as percentage of inhibition between vehicle (DMSO, 0% inhibition) and no enzyme (100% inhibition). The percentage inhibition is fitted against the compound dilutions with a four-parameter logistic regression. The inflection point (i.e. the concentration at which half-maximal inhibition is achieved) is the IC₅₀.

The IC₅₀ results of the compounds of the invention in the SHP2 inhibition enzymatic assay are shown in Table 2. Legend: A indicates IC₅₀ less or equal to 0.5 μM; B indicates IC₅₀ greater than 0.5 μM and lower or equal to 3 μM; C indicates IC₅₀ higher than 3 μM.

TABLE 2
SHP2 inhibition for compounds of the invention

| Example | SHP2wt-IC₅₀ |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | B |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | B |
| 30 | A |
| 31 | A |
| 32 | A |
| 33 | A |
| 34 | A |
| 35 | B |
| 36 | A |
| 37 | A |
| 38 | A |
| 39 | A |
| 40 | A |
| 41 | A |
| 42 | B |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | B |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | B |
| 131 | A |
| 132 | A |
| 133 | A |
| 134 | A |
| 135 | C |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | A |
| 285 | A |
| 286 | A |
| 287 | A |
| 288 | A |
| 289 | A |
| 290 | A |
| 291 | A |
| 292 | A |
| 293 | A |
| 294 | A |
| 295 | A |
| 296 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | A |
| 348 | A |
| 349 | A |
| 350 | A |
| 351 | A |
| 352 | A |
| 353 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | C |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | C |
| 384 | A |
| 385 | A |
| 386 | A |
| 387 | A |
| 388 | A |
| 389 | A |
| 390 | A |
| 391 | A |
| 392 | A |
| 393 | A |
| 394 | C |
| 395 | A |
| 396 | A |
| 397 | A |
| 398 | A |
| 399 | A |
| 400 | A |
| 401 | A |
| 402 | A |
| 403 | A |
| 404 | A |
| 405 | A |
| 406 | A |
| 407 | A |
| 408 | A |
| 409 | A |
| 410 | B |
| 411 | C |
| 412 | A |
| 413 | A |
| 414 | A |
| 415 | A |
| 416 | A |
| 417 | A |
| 418 | A |
| 419 | A |
| 420 | A |
| 421 | A |
| 422 | A |
| 423 | A |
| 424 | A |
| 425 | A |
| 426 | A |
| 427 | A |
| 428 | A |
| 429 | A |
| 430 | A |
| 431 | A |
| 432 | A |
| 433 | A |
| 434 | A |
| 435 | A |
| 436 | A |
| 437 | A |
| 438 | A |
| 439 | A |
| 440 | A |
| 441 | A |
| 442 | A |
| 443 | B |
| 444 | A |
| 445 | A |
| 446 | A |
| 447 | A |
| 448 | A |
| 449 | A |
| 450 | A |
| 451 | A |
| 452 | A |
| 453 | A |
| 454 | A |
| 455 | A |
| 456 | A |
| 457 | A |
| 458 | A |
| 459 | A |
| 460 | A |
| 461 | A |
| 462 | A |
| 463 | A |
| 464 | A |
| 465 | A |
| 466 | A |
| 467 | A |
| 468 | A |
| 469 | A |
| 470 | A |
| 471 | A |
| 472 | A |
| 473 | A |
| 474 | A |
| 475 | A |
| 476 | A |
| 477 | A |
| 478 | A |
| 479 | A |
| 480 | A |
| 481 | A |
| 482 | A |
| 483 | A |
| 484 | A |
| 485 | A |
| 486 | A |
| 487 | A |
| 488 | A |
| 489 | A |
| 490 | A |
| 491 | A |
| 492 | A |
| 493 | A |
| 494 | A |
| 495 | A |
| 496 | A |
| 497 | A |
| 498 | A |
| 499 | A |
| 500 | A |
| 501 | A |
| 502 | A |
| 503 | A |
| 504 | A |
| 505 | A |
| 506 | A |
| 507 | A |
| 508 | A |
| 509 | A |
| 510 | A |
| 511 | A |
| 512 | A |
| 513 | A |
| 514 | A |
| 515 | A |
| 516 | A |
| 517 | A |
| 518 | A |
| 519 | A |
| 520 | A |
| 521 | A |
| 522 | A |
| 523 | A |
| 524 | A |
| 525 | A |
| 526 | A |
| 527 | A |
| 528 | A |
| 529 | A |
| 530 | A |
| 531 | A |

### Phospho-ERK Cellular Assay

ERK phosphorylation was detected using the “Advanced phospho-ERK1/2 (Thr202/Tyr204)” TR-FRET kit (Cisbio, Cat #64AERPEG/H), following the manufacturer reagents and instructions.

Briefly, 20.000/well KYSE-520 cells (DSMZ ACC 371) were plated in 6 μL RPMI-1640 (Invitrogen) growth medium, into 384 white low-volume high base TC microplates (Greiner). After an overnight incubation, cells were treated with DMSO serial diluted compounds and incubated for 2 h at 37° C. After incubation, 2 μL/well of 4×Lysis Buffer (Cisbio 64KL1FDF), were added and incubated with cells for 30 min on gentle shaking. Finally, lysates were added with 2 μL/well of Eu cryptate (donor) and D2 (acceptor) conjugated antibodies (as provided by the Cisbio kit #64AERPEG/H) diluted 1:100 in Detection Buffer (as provided by the Cisbio kit #64AERPEG/H). Plates were then sealed and incubated at room temperature in the dark. After an overnight incubation, the TR-FRET signal was detected on a suitable reader (Envision, PerkinElmer). The lower the TR-FRET signal the higher the higher the SHP2 inhibition in cells.

The activity of each compound dilution was calculated as percentage between vehicle DMSO treated cells and no cells, 0% inhibition and 100% inhibition respectively. The percentage activity is fitted against the compound dilutions with a four-parameter logistic regression. The inflection point (i.e. the concentration at which half-maximal inhibition is achieved) is the IC₅₀.

The IC₅₀ results of the compounds of the invention in the phospho-ERK cellular assay are shown in Table 3. Legend: “+” indicates IC₅₀ equal or higher than 5 μM; “++” indicates IC₅₀ less than 5 μM and higher or equal to 1 μM; “+++” indicates IC₅₀ less than 1 μM.

TABLE 2
pERK activity for compounds of the invention

| Example | PERK IC₅₀ |
|---|---|
| 1 | + |
| 2 | +++ |
| 3 | +++ |
| 4 | ++ |
| 5 | +++ |
| 6 | +++ |
| 7 | +++ |
| 8 | +++ |
| 9 | +++ |
| 10 | +++ |
| 11 | + |
| 12 | + |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | +++ |
| 23 | +++ |
| 24 | +++ |
| 25 | +++ |
| 26 | +++ |
| 27 | +++ |
| 28 | + |
| 29 | + |
| 30 | +++ |
| 31 | +++ |
| 32 | +++ |
| 33 | +++ |
| 34 | +++ |
| 35 | ++ |
| 36 | +++ |
| 37 | ++ |
| 38 | +++ |
| 39 | +++ |
| 40 | +++ |
| 41 | +++ |
| 42 | ++ |
| 43 | +++ |
| 44 | +++ |
| 45 | +++ |
| 46 | +++ |
| 47 | +++ |
| 48 | +++ |
| 49 | +++ |
| 50 | +++ |
| 51 | +++ |
| 52 | +++ |
| 53 | +++ |
| 54 | +++ |
| 55 | +++ |
| 56 | +++ |
| 57 | ++ |
| 58 | + |
| 59 | +++ |
| 60 | +++ |
| 61 | +++ |
| 62 | ++ |
| 63 | ++ |
| 64 | ++ |
| 65 | ++ |
| 66 | +++ |
| 67 | +++ |
| 68 | + |
| 69 | ++ |
| 70 | +++ |
| 71 | + |
| 72 | +++ |
| 73 | ++ |
| 74 | ++ |
| 75 | + |
| 76 | + |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | ++ |
| 96 | +++ |
| 97 | +++ |
| 98 | +++ |
| 99 | +++ |
| 100 | +++ |
| 101 | +++ |
| 102 | +++ |
| 103 | +++ |
| 104 | + |
| 105 | +++ |
| 106 | +++ |
| 107 | +++ |
| 108 | +++ |
| 109 | +++ |
| 110 | +++ |
| 111 | +++ |
| 112 | +++ |
| 113 | +++ |
| 114 | +++ |
| 115 | +++ |
| 116 | +++ |
| 117 | +++ |
| 118 | +++ |
| 119 | +++ |
| 120 | +++ |
| 121 | +++ |
| 122 | +++ |
| 123 | +++ |
| 124 | +++ |
| 125 | +++ |
| 126 | +++ |
| 127 | +++ |
| 128 | +++ |
| 129 | +++ |
| 130 | +++ |
| 131 | +++ |
| 132 | +++ |
| 133 | +++ |
| 134 | +++ |
| 135 | + |
| 136 | +++ |
| 137 | +++ |
| 138 | +++ |
| 139 | +++ |
| 140 | +++ |
| 141 | +++ |
| 142 | +++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | +++ |
| 153 | +++ |
| 154 | +++ |
| 155 | +++ |
| 156 | +++ |
| 157 | +++ |
| 158 | +++ |
| 159 | +++ |
| 160 | +++ |
| 161 | +++ |
| 162 | +++ |
| 163 | +++ |
| 164 | +++ |
| 165 | +++ |
| 166 | +++ |
| 167 | +++ |
| 168 | +++ |
| 169 | +++ |
| 170 | +++ |
| 171 | +++ |
| 172 | +++ |
| 173 | +++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | +++ |
| 185 | +++ |
| 186 | +++ |
| 187 | +++ |
| 188 | +++ |
| 189 | +++ |
| 190 | +++ |
| 191 | +++ |
| 192 | + |
| 193 | +++ |
| 194 | +++ |
| 195 | +++ |
| 196 | +++ |
| 197 | +++ |
| 198 | +++ |
| 199 | +++ |
| 200 | +++ |
| 201 | +++ |
| 202 | +++ |
| 203 | +++ |
| 204 | +++ |
| 205 | +++ |
| 206 | +++ |
| 207 | +++ |
| 208 | +++ |
| 209 | +++ |
| 210 | +++ |
| 211 | + |
| 212 | + |
| 213 | +++ |
| 214 | + |
| 215 | +++ |
| 216 | +++ |
| 217 | +++ |
| 218 | +++ |
| 219 | +++ |
| 220 | +++ |
| 221 | +++ |
| 222 | +++ |
| 223 | +++ |
| 224 | +++ |
| 225 | +++ |
| 226 | +++ |
| 227 | + |
| 228 | +++ |
| 229 | +++ |
| 230 | +++ |
| 231 | +++ |
| 232 | +++ |
| 233 | +++ |
| 234 | +++ |
| 235 | + |
| 236 | +++ |
| 237 | +++ |
| 238 | +++ |
| 239 | +++ |
| 240 | +++ |
| 241 | +++ |
| 242 | +++ |
| 243 | +++ |
| 244 | +++ |
| 245 | +++ |
| 246 | +++ |
| 247 | +++ |
| 248 | +++ |
| 249 | + |
| 250 | +++ |
| 251 | +++ |
| 252 | +++ |
| 253 | +++ |
| 254 | +++ |
| 255 | + |
| 256 | +++ |
| 257 | + |
| 258 | + |
| 259 | +++ |
| 260 | + |
| 261 | +++ |
| 262 | +++ |
| 263 | +++ |
| 264 | +++ |
| 265 | +++ |
| 266 | +++ |
| 267 | +++ |
| 268 | +++ |
| 269 | + |
| 270 | +++ |
| 271 | + |
| 272 | +++ |
| 273 | +++ |
| 274 | +++ |
| 275 | +++ |
| 276 | +++ |
| 277 | + |
| 278 | +++ |
| 279 | +++ |
| 280 | +++ |
| 281 | + |
| 282 | +++ |
| 283 | +++ |
| 284 | +++ |
| 285 | + |
| 286 | + |
| 287 | +++ |
| 288 | + |
| 289 | +++ |
| 290 | +++ |
| 291 | +++ |
| 292 | + |
| 293 | +++ |
| 294 | +++ |
| 295 | +++ |
| 296 | +++ |
| 297 | +++ |
| 298 | +++ |
| 299 | +++ |
| 300 | +++ |
| 301 | +++ |
| 302 | +++ |
| 303 | +++ |
| 304 | +++ |
| 305 | +++ |
| 306 | +++ |
| 307 | +++ |
| 308 | +++ |
| 309 | + |
| 310 | + |
| 311 | +++ |
| 312 | +++ |
| 313 | +++ |
| 314 | +++ |
| 315 | +++ |
| 316 | +++ |
| 317 | +++ |
| 318 | +++ |
| 319 | +++ |
| 320 | +++ |
| 321 | +++ |
| 322 | +++ |
| 323 | +++ |
| 324 | +++ |
| 325 | +++ |
| 326 | +++ |
| 327 | +++ |
| 328 | + |
| 329 | +++ |
| 330 | +++ |
| 331 | +++ |
| 332 | +++ |
| 333 | +++ |
| 334 | +++ |
| 335 | +++ |
| 336 | +++ |
| 337 | +++ |
| 338 | +++ |
| 339 | +++ |
| 340 | +++ |
| 341 | +++ |
| 342 | + |
| 343 | + |
| 344 | +++ |
| 345 | + |
| 346 | + |
| 347 | + |
| 348 | +++ |
| 349 | +++ |
| 350 | +++ |
| 351 | +++ |
| 352 | +++ |
| 353 | +++ |
| 354 | +++ |
| 355 | +++ |
| 356 | + |
| 357 | +++ |
| 358 | +++ |
| 359 | +++ |
| 360 | +++ |
| 361 | +++ |
| 362 | +++ |
| 363 | +++ |
| 364 | +++ |
| 365 | +++ |
| 366 | +++ |
| 367 | +++ |
| 368 | +++ |
| 369 | +++ |
| 370 | +++ |
| 371 | + |
| 372 | +++ |
| 373 | +++ |
| 374 | +++ |
| 375 | +++ |
| 376 | +++ |
| 377 | +++ |
| 378 | +++ |
| 379 | + |
| 380 | +++ |
| 381 | + |
| 382 | + |
| 383 | +++ |
| 384 | +++ |
| 385 | +++ |
| 386 | +++ |
| 387 | +++ |
| 388 | +++ |
| 389 | +++ |
| 390 | + |
| 391 | +++ |
| 392 | +++ |
| 393 | +++ |
| 394 | +++ |
| 395 | +++ |
| 396 | +++ |
| 397 | +++ |
| 398 | +++ |
| 399 | +++ |
| 400 | +++ |
| 401 | +++ |
| 402 | +++ |
| 403 | + |
| 404 | +++ |
| 405 | +++ |
| 406 | +++ |
| 407 | +++ |
| 408 | + |
| 409 | +++ |
| 410 | + |
| 411 | + |
| 412 | +++ |
| 413 | +++ |
| 414 | +++ |
| 415 | +++ |
| 416 | + |
| 417 | +++ |
| 418 | +++ |
| 419 | +++ |
| 420 | +++ |
| 421 | +++ |
| 422 | +++ |
| 423 | + |
| 424 | +++ |
| 425 | +++ |
| 426 | +++ |
| 427 | +++ |
| 428 | +++ |
| 429 | +++ |
| 430 | +++ |
| 431 | +++ |
| 432 | +++ |
| 433 | +++ |
| 434 | +++ |
| 435 | +++ |
| 436 | +++ |
| 437 | ++ |
| 438 | +++ |
| 439 | +++ |
| 440 | +++ |
| 441 | +++ |
| 442 | +++ |
| 443 | +++ |
| 444 | +++ |
| 445 | +++ |
| 446 | +++ |
| 447 | +++ |
| 448 | +++ |
| 449 | +++ |
| 450 | +++ |
| 451 | +++ |
| 452 | +++ |
| 453 | +++ |
| 454 | +++ |
| 455 | +++ |
| 456 | +++ |
| 457 | +++ |
| 458 | +++ |
| 459 | +++ |
| 460 | +++ |
| 461 | +++ |
| 462 | +++ |
| 463 | +++ |
| 464 | +++ |
| 465 | +++ |
| 466 | +++ |
| 467 | +++ |
| 468 | +++ |
| 469 | +++ |
| 470 | +++ |
| 471 | +++ |
| 472 | +++ |
| 473 | +++ |
| 474 | +++ |
| 475 | +++ |
| 476 | +++ |
| 477 | +++ |
| 478 | +++ |
| 479 | +++ |
| 480 | +++ |
| 481 | +++ |
| 482 | +++ |
| 483 | +++ |
| 484 | +++ |
| 485 | +++ |
| 486 | +++ |
| 487 | +++ |
| 488 | +++ |
| 489 | +++ |
| 490 | +++ |
| 491 | +++ |
| 492 | +++ |
| 493 | +++ |
| 494 | +++ |
| 495 | +++ |
| 496 | +++ |
| 497 | +++ |
| 498 | +++ |
| 499 | +++ |
| 500 | +++ |
| 501 | +++ |
| 502 | +++ |
| 503 | +++ |
| 504 | +++ |
| 505 | +++ |
| 506 | +++ |
| 507 | +++ |
| 508 | +++ |
| 509 | +++ |
| 510 | +++ |
| 511 | +++ |
| 512 | +++ |
| 513 | +++ |
| 514 | +++ |
| 515 | +++ |
| 516 | +++ |
| 517 | +++ |
| 518 | +++ |
| 519 | +++ |
| 520 | +++ |
| 521 | +++ |
| 522 | +++ |
| 523 | +++ |
| 524 | + |
| 525 | + |
| 526 | + |
| 527 | ++ |
| 528 | +++ |
| 529 | +++ |
| 530 | + |
| 531 | N.D. |

1. A compound of Formula (I): 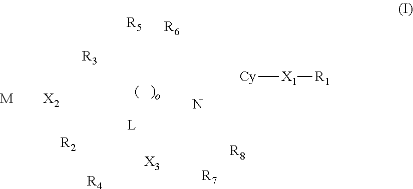
Wherein
X₁ is S, O, CR₁ₐR_{1b}, NH, N—CH₃ or a single bond;
X₂ is (CR₂ₐR_{2b})ₘ;
X₃ is (CR₃ₐR_{3b})ₙ;
M is NH₂, NH—C₁₋₆alkyl, N(C₁₋₆alkyl)₂, NHC(═O)Rₓ or NHC(═O)ORₓ wherein Rₓ is C₁₋₆alkyl, aryl, heteroaryl or C₁₋₆alkyl-aryl;
L is a single bond or C₁₋₃alkyl;
Cy-X₁—R₁ has general formula (A): 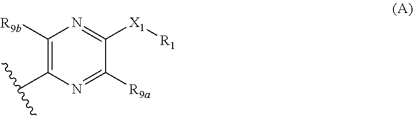
wherein
R₉ₐ is selected from H, NH₂, C₁₋₆alkyl, halogen, cyano, C₃₋₅cycloalkyl, haloC₁₋₆alkyl, hydroxy-C₁₋₆ alkyl or aminoC₁₋₆alkyl;
R_{9b} is selected from H, C₁₋₆alkyl, halogen, haloC₁₋₆alkyl, hydroxy-C₁₋₆alkyl or aminoC₁₋₆alkyl;
or Cy-X₁—R₁ has general formula (B): 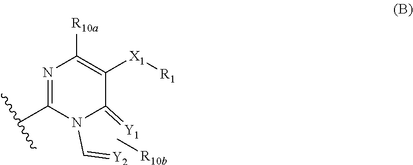
wherein
Y₁ and Y₂ are C or N;
R₁₀ₐ is H or C₁₋₆alkyl;
R_{10b} is at each occurrence independently H, C₁₋₆alkyl, NH₂, CN, OH, haloC₁₋₃alkyl, C₁₋₃alkoxy, haloC₁₋₃alkoxy, or halogen;
or Cy-X₁—R₁ has general formula (C): 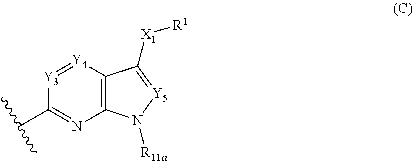
wherein
Y₃ is C—R_{11b} or N;
Y₄ is C—R_{11b} or N; or Y₃ is N—CH₃ and Y₄ is C(═O);
Y₅ is C—R_{11c} or N;
Y₃ [symbol] Y₄ is a double bond when Y₃ is C—R_{11b} or N and when Y₄ is C—R_{11b} or N; Y₃ [symbol] Y₄ is a single bond when Y₃ is N—CH₃ and Y₄ is C(═O);
R₁₁ₐ is H or methyl;
R_{11b} is H, C₁₋₆alkyl, hydroxyC₁₋₆alkyl;
R_{11c} is H, C₁₋₆alkyl, halogen;
or Cy-X₁—R₁ has general formula (D): 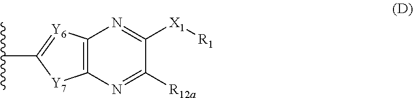
wherein
Y₆ is N, CH or C—CH₃;
Y₇ is N—R_{12b}, O or S;
R₁₂ₐ is H, C₁₋₆alkyl, halogen or cyano;
R_{12b} is H or C₁₋₆alkyl;
or Cy-X₁—R₁ has general formula (E): 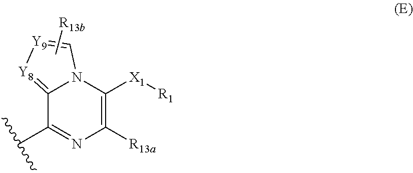
wherein
Y₉ and Y₉ are C or N;
R₁₃ₐ is H, C₁₋₆alkyl, halogen, NH₂ or cyano;
R_{13b} is at each occurrence independently H, CH₃ or hydroxyC₁₋₆alkyl;
or Cy-X₁—R₁ has general formula (F): 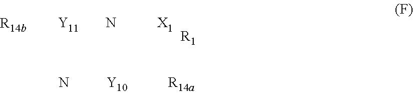
wherein
Y₁₀ and Y₁₁ are C or N;
R₁₄ₐ is H, C₁₋₆alkyl, halogen;
R_{14b} is H, C₁₋₆alkyl or hydroxyC₁₋₆alkyl;
or Cy-X₁—R₁ has general formula (G): 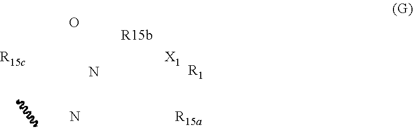
wherein
R₁₅ₐ and R_{15b} are each independently H, C₁₋₆alkyl, halogen;
R_{15c} is H, C₁₋₆alkyl or hydroxyC₁₋₆alkyl;
or Cy-X₁—R₁ has general formula (H): 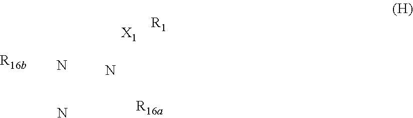
wherein
R₁₆ₐ is H, C₁₋₆alkyl or halogen;
R_{16b} is H, C₁₋₆alkyl or hydroxyC₁₋₆alkyl;
or Cy-X₁—R₁ has general formula (I): 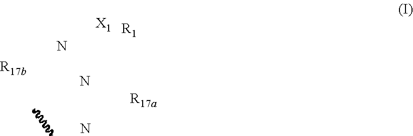
wherein
R₁₇ₐ is H, C₁₋₆alkyl or halogen;
R_{17b} is H, C₁₋₆alkyl or hydroxyC₁₋₆alkyl;
or Cy-X₁—R₁ has general formula (J): 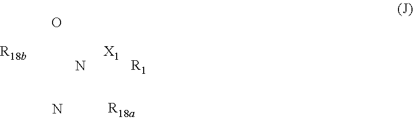
wherein
R₁₈ₐ is H, C₁₋₆alkyl;
R_{18b} is H, halogen, C₁₋₆alkyl or hydroxyC₁₋₆alkyl;
R₁ is an aryl, heteroaryl, partially unsaturated aryl, partially unsaturated heteroaryl or an heterocyclic ring, each of said aryl, heteroaryl, partially unsaturated aryl, partially unsaturated heteroaryl or heterocyclic ring being optionally substituted with one or more substituents independently selected from C(O)CH₃, C(O)OCH₃, heteroaryl, aryl, OH, halogen, NH₂, NH—C₁₋₆alkyl, N(C₁₋₆alkyl)₂, NH—C₃₋₆cycloalkyl, C(O)NH₂, C(O)NHC₁₋₃alkyl, C(O)N(C₁₋₃alkyl)₂, C₁₋₆alkylC(O)NH₂, C(O)NHSO₂CH₃, C(O)NHCN, C(O)N(CH₃)CN, SO₂NH₂, OC(O)CH₃, C₁₋₆alkyl, C₃₋₅cycloalkyl, C₁₋₆alkyl-NH₂, C₁₋₆alkyl-NH(CH₃), C₁₋₆alkyl-NH(CH₃)₂, C₁₋₆alkyl-NHCOCH₃, C₁₋₃alkylaryl, C₁₋₃alkylheteroaryl, haloC₁₋₆alkyl, hydroxyC₁₋₆alkyl, CN, haloC₁₋₆alkoxy, C₁₋₆alkoxy, C₅₋₇heterocycloalkoxy, oxetane, OCF₃, CH₂OCH₃, OSO₂F, SO₂F, C₁₋₃alkyl-OC₁₋₃alkyl, heterocycloalkyl, heteroaryl-OH, and a cyclic amine selected from the group consisting of: pyrrolidine, piperidine, morpholine, thiomorpholine, piperazine, N-methylpyperazine and pyrrolidin-3-yloxy, pyrrolidine-2-one wherein said cyclic amine is optionally substituted with C₁₋₃alkyl, C₁₋₃alkyl-OH;
or R₁ has general formula (K) 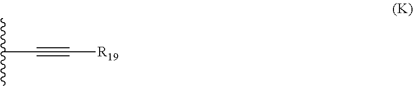
wherein R₁₉ is C₁₋₆alkyl, aryl, heteroaryl and said aryl or heteroaryl are optionally substituted with one or more substituents independently selected from halogen, hydroxy, C₁₋₃alkyl, C₁₋₃alkoxy;
R₂ is aryl, heteroaryl, C₃₋₉cycloalkyl, partially unsaturated heteroaryl, C₁₋₃alkylaryl, C₁₋₃alkylheteroaryl, each of said aryl, heteroaryl, C₃₋₉cycloalkyl, partially unsaturated heteroaryl, C₁₋₃alkylaryl, C₁₋₃alkylheteroaryl being optionally substituted with one or more substituents independently selected from the group consisting of: halogen, CN, C₁₋₃alkyl, C₃₋₈cycloalkyl, haloC₁₋₃alkyl, C₁₋₃alkoxy, haloC₁₋₃alkoxy, C₁₋₃alkyl-N(R_{y})₂, C(O)OC₁₋₃alkyl, C(O)C₁₋₃alkyl, OH, hydroxy-C₁₋₃alkyl, C₁₋₃alkyl-OC₁₋₃alkyl, CON(R_{y})₂, N(R_{y})₂ wherein each R_{y} is independently selected from H, methyl, ethyl and cyclopropyl; or R₂ is C(O)NH₂, C(O)NHC₁₋₃alkyl or C(O)N(C₁₋₃alkyl)₂;
R₁ₐ and R_{1b} are independently selected from H, C₁₋₆alkyl or R₁ₐ and R_{1b} are joined together to form together with the carbon atom to which they are linked a spiro-C₃₋₅cycloalkyl ring;
R₂ₐ and R_{2b} are independently selected from H, halogen, C₁₋₆alkyl, C₃₋₅cycloalkyl or R₃ₐ and R_{3b} are joined together to form together with the carbon atom to which they are linked a spiro-C₃₋₅cycloalkyl ring;
R₃, R₃ₐ, R_{3b}, R₄, R₅, R₆, R₇ and R₈ are at each occurrence independently selected from H, methyl or halogen;
m is 0, 1, 2 or 3;
n is 0, 1 or 2;
o is 1 or 2; preferably o is 1;
or a pharmaceutically acceptable salt, tautomer, solvate, or stereoisomer thereof.

2. The compound according to claim 1 having general formula (II), (III), (IV), (V), (VI), (VII), (VIII), (IX), (X), (XI), (XII), (XIII), (XIV), (XV), (XVIa) or (XVIb): 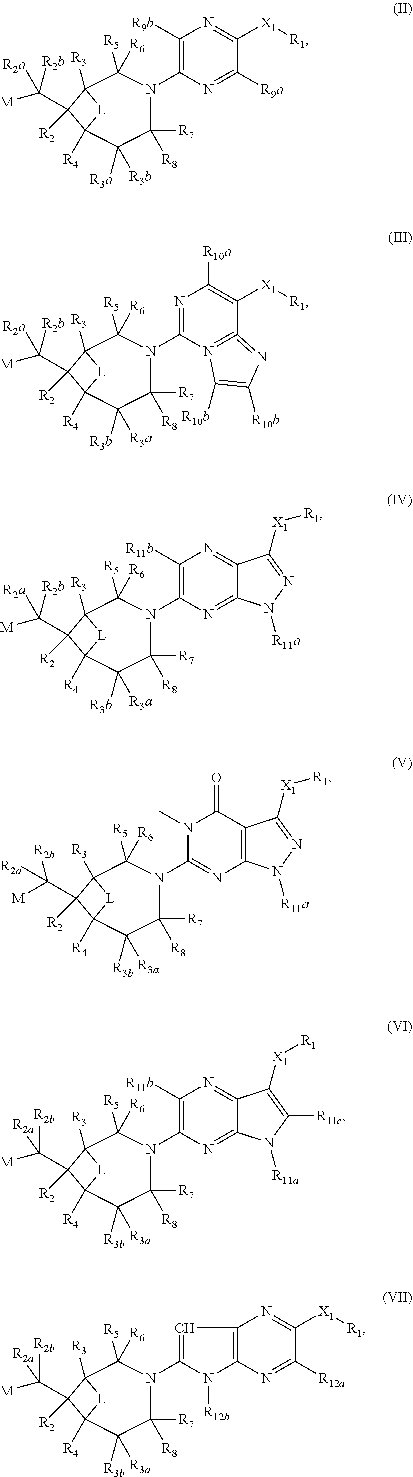 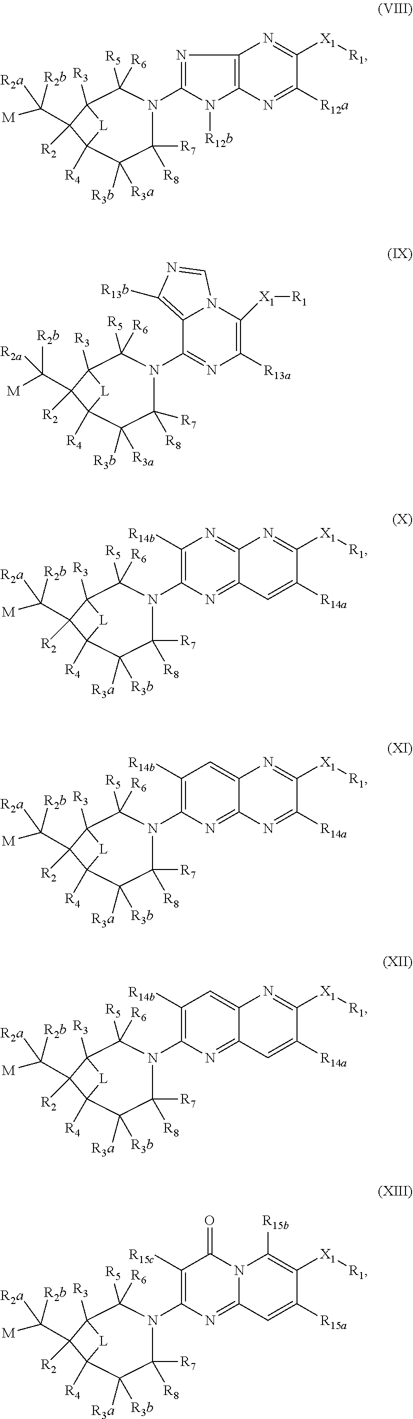 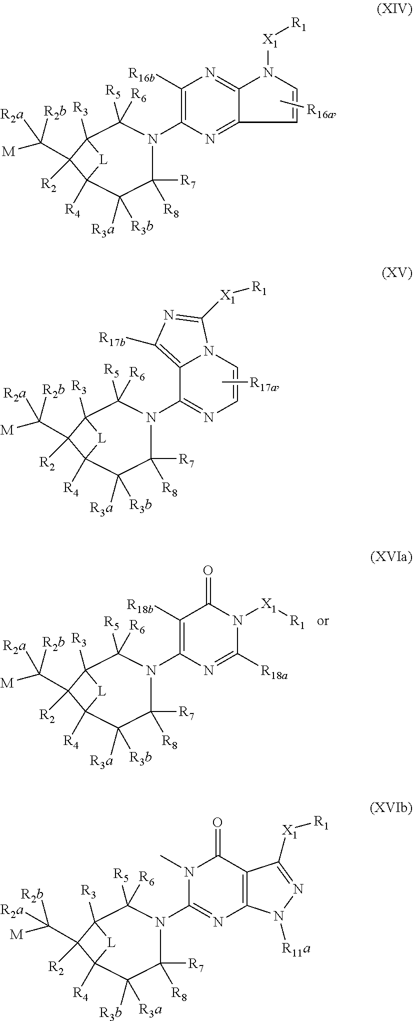
or a pharmaceutically acceptable salt, tautomer, solvate, or stereoisomer thereof.

3. The compound according to claim 1 having general formula (XVII), (XVIII), (XIX), (XX), (XXI), (XXII), (XXIII), (XXIV), (XXV), (XXVI), (XXVII), (XXVIII), (XIX), (XXX), (XXXI) or (XXXII): 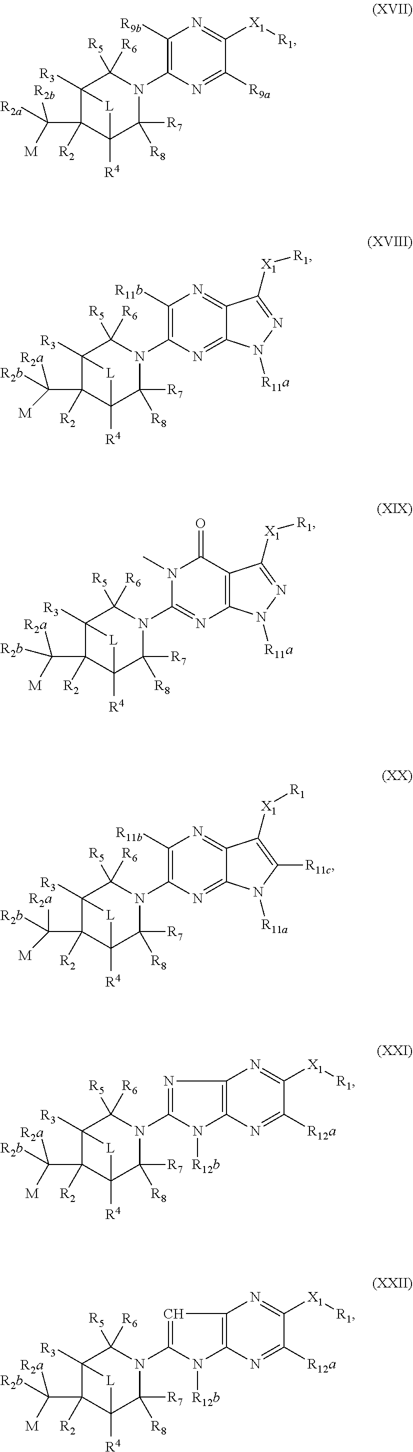 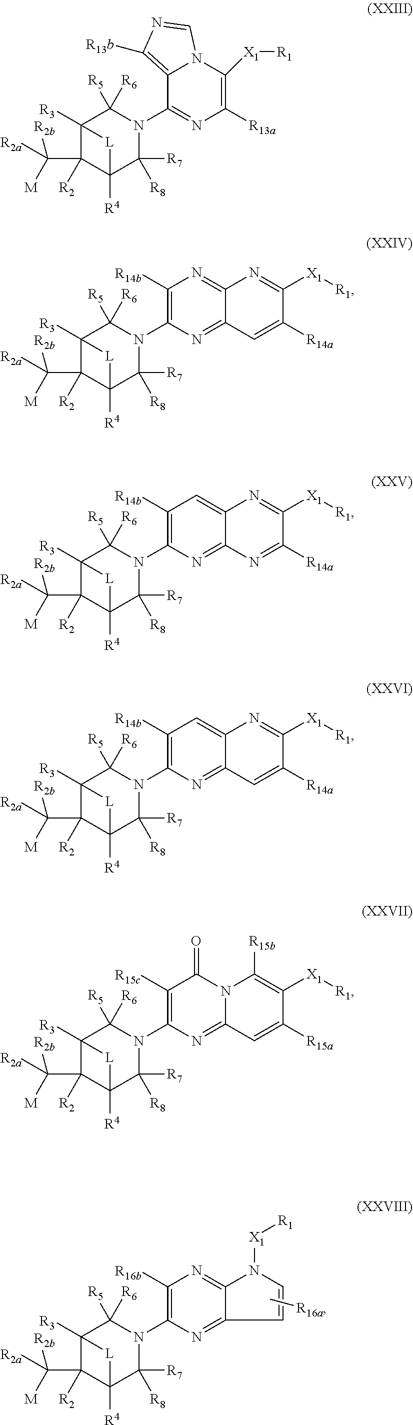 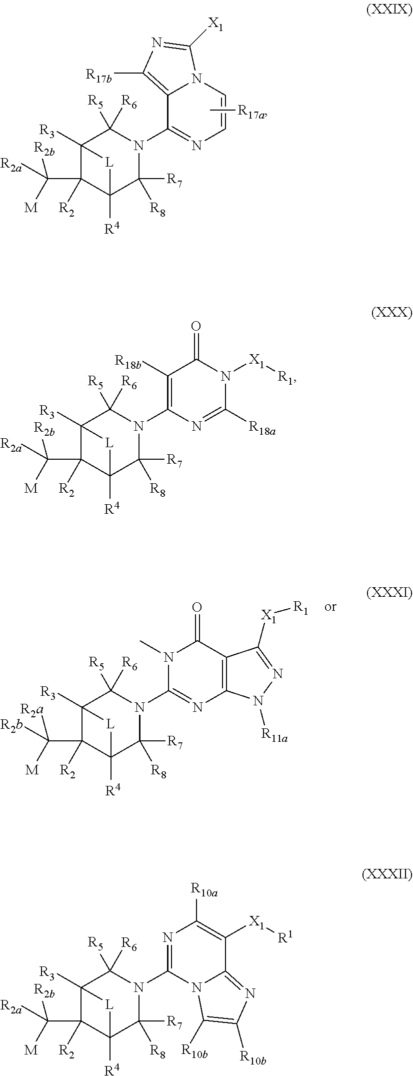
or a pharmaceutically acceptable salt, tautomer, solvate, or stereoisomer thereof.

4. The compound according to claim 1 wherein:
L is a single bond; and/or
X₁ is a single bond or S;
or a pharmaceutically acceptable salt, tautomer, solvate, or stereoisomer thereof.

5. The compound according to claim 1 wherein M is NH₂; or a pharmaceutically acceptable salt, tautomer, solvate, or stereoisomer thereof.

6. The compound according to claim 1 wherein R₂ₐ, R_{2b}, R₃, R₃ₐ, R_{3b}, R₄, R₅, R₆, R₇, R₈ are H.

7. The compound according to claim 1 having formula (I-A): 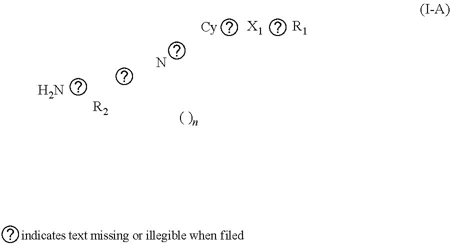
wherein n is 0 or 1;
or a pharmaceutically acceptable salt, tautomer, solvate, or stereoisomer thereof.

8. The compound according to claim 7 having one of the following formulae: 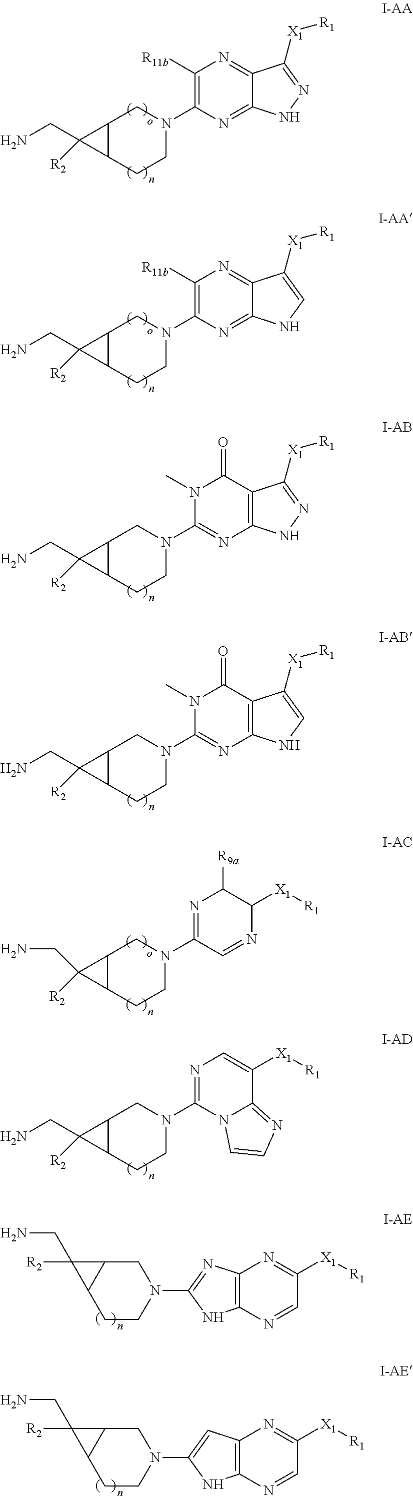 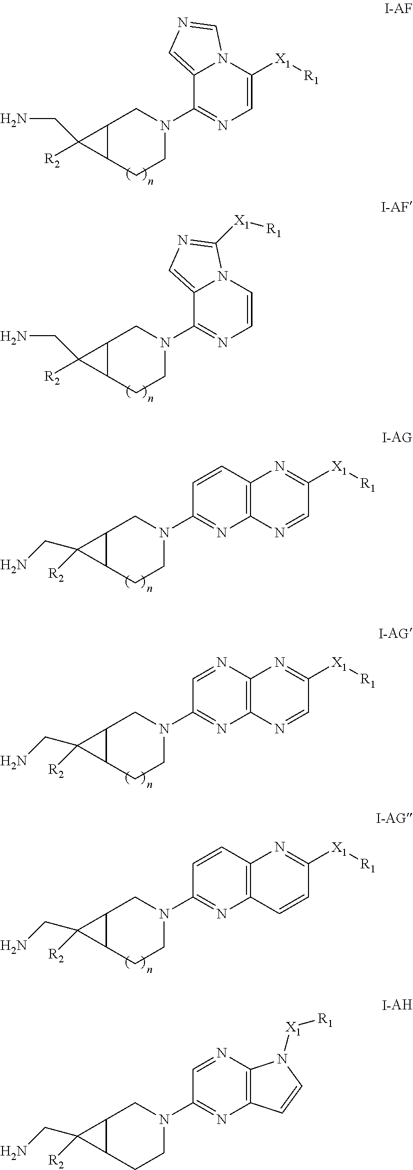 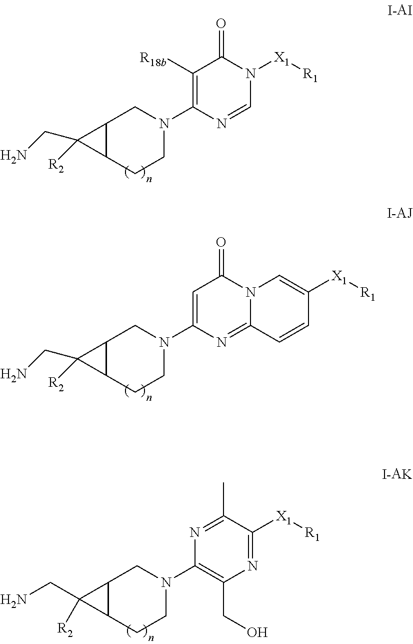
or a pharmaceutically acceptable salt, tautomer, solvate, or stereoisomer thereof.

9. The compound according to claim 1 wherein R₁ is selected from: phenyl, napthyl, benzothiophene, quinoline, quinoxaline, isoquinoline, pyrazolo[3,4-b]pyrazine, pyridine, pyrimidine, pyrazine, thienopyridine, indole, indoline, 2,3-dihydroisoindol-1-one, indol-2-one, benzothiazole, 6a,7,8,9-tetrahydro-6H-pyrido[3,2-b]pyrrolo[1,2-d][1,4]oxazine, napthyridine, 1,2,3,4-tetrahydro-1,5-naphthyridine, benzo[d][1,3]dioxole, 2,3-dihydrobenzo[b][1,4]dioxine, benzoxazole, thieno[3,2-b]pyridine, indazole, furo[2,3-b]pyridine, 2,1,3-benzoxadiazole, benzo[d]oxazol-2(3H)-one, pyridine-2(1H)one, benzo[d][1,2,3]triazole, 1,4-benzoxazin-3(4H)-one, isoquinolin-(2H)one, quinolin-(2H)one, [1,2,4]triazolo[4,3-a]pyridine, imidazo[1,2-a]pyridine, 1,3-dihydro-2H-benzimidazol-2-one, benzoisothiazole-3(2H)-one-1,1-dioxide, benzo[d]thiazol-2(3H)-one phthalazine-1(2H)-one, pyrido[1,2-a]pyrimidin-4-one, cinnoline, [1,2,4]triazolo[1,5-a]pyridine, benzimidazole, indolin-2,3-dione, each being optionally substituted with one or more substituents independently selected form halogen, C₁₋₃ alkyl, CN, CF₃, CHF₂, NH₂, OCH₃, OCF₃, CH₂OCH₃, OSO₂F, SO₂F, oxetane, C(CH₃)₂OH, CH(CH₃)OH, CH(CH₃)OCH₃, heteroaryl, heteroaryl-C₁₋₃alkyl-substituted, heteroaryloxy, C(O)N(CH₃)₂, C(O)NH₂, C(O)NHCH₃, C(O)NHcyclopropyl, C(O)NHCN, C(O)N(CH₃)CN CONHSO₂CH₃, C(O)OCH₃, COOH, cyclobutane, OH, CH₃C(O), CH₂C(O)NH₂, CH₂C(O)NH(CH₃), CH₂C(O)N(CH₃)₂, pyrrolidine-2-one, pyrrolidine-3-yl-methanol, CH₂CH₂—OH, CH₂CH₂—OCH₃, SO₂NH₂, OC(O)CH₃, CH₂CF₃, or CH₂CN.

10. The compound according to claim 1 wherein R₂ is selected from: phenyl, pyridine, pyrimidine, pyrazine, oxazole, isoxazole, pyrrole, pyrazole triazole, oxadiazole, thiophene, thiazole, thiadiazole, imidazole, each optionally substituted with one or more substituents independently selected form halogen, C₁₋₃alkyl, C₃₋₈cycloalkyl, CN, CF₃, NH₂, OCH₃, OH, CH₂OH, CH₂OCH₃, CO₂CH₃, CONHC₁₋₆alkyl, or R₂ is CON(CH₃)₂ or C(O)NHCH₃.

11. A compound according to claim 1 selected from the following list:
(3-(7-(aminomethyl)-7-(thiophen-3-yl)-3-azabicyclo[4.1.0]heptan-3-yl)-6-(2,3-dichlorophenyl)-5-methylpyrazin-2-yl)methanol
(3-(3-(2,3-dichlorophenyl)-1H-pyrazolo[3,4-b]pyrazin-6-yl)-7-(pyridin-3-yl)-3-azabicyclo[4.1.0]heptan-7-yl)methanamine
(3-(3-(2,3-dichlorophenyl)-1H-pyrazolo[3,4-b]pyrazin-6-yl)-7-(2-fluorophenyl)-3-azabicyclo[4.1.0]heptan-7-yl)methanamine
(3-(3-(benzo[b]thiophen-7-yl)-1H-pyrazolo[3,4-b]pyrazin-6-yl)-7-(2-fluorophenyl)-3-azabicyclo[4.1.0]heptan-7-yl)methanamine
(7-(2-fluorophenyl)-3-(3-(quinolin-5-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(3-methylisoxazol-5-yl)-3-azabicyclo[4.1.0]heptan-7-yl)methanamine
(3-(3-(2,3-dichlorophenyl)-1H-pyrazolo[3,4-b]pyrazin-6-yl)-7-(3-fluorophenyl)-3-azabicyclo[4.1.0]heptan-7-yl)methanamine
(3-(3-(2,3-dichlorophenyl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(quinolin-5-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(4-fluorophenyl)-3-azabicyclo[4.1.0]heptan-7-yl)methanamine
(7-(4-fluorophenyl)-3-(3-(quinoxalin-6-yl)-1H-pyrazolo[3,4-b]pyrazin-6-yl)-3-azabicyclo[4.1.0]heptan-7-yl)methanamine
(7-(4-fluorophenyl)-3-(3-(quinolin-8-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-1,2,3-triazol-4-yl)-3-azabicyclo[4.1.0]heptan-7-yl)methanamine
(3-(3-(2,3-dichlorophenyl)-1H-pyrazolo[3,4-b]pyrazin-6-yl)-7-(4-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(4-methylisoxazol-3-yl)-3-azabicyclo[4.1.0]heptan-7-yl)methanamine
4-(6-(7-(aminomethyl)-7-(2-fluorophenyl)-3-azabicyclo[4.1.0]heptan-3-yl)-1H-pyrazolo[3,4-b]pyrazin-3-yl)-3-chloro-N-methylpyridin-2-amine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)-3-chloro-N-methylpyridin-2-amine
(7-(5-methylisoxazol-3-yl)-3-(3-(thieno[3,2-b]pyridin-7-yl)-1H-pyrazolo[3,4-b]pyrazin-6-yl)-3-azabicyclo[4.1.0]heptan-7-yl)methanamine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)isoindolin-1-one
(3-(3-(benzo[d]thiazol-5-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(8-methylquinolin-5-yl)-1H-pyrazolo[3,4-b]pyrazin-6-yl)-3-azabicyclo[4.1.0]heptan-7-yl)methanamine
(3-(3-(8-methoxyquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(isoquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(2-methylquinolin-5-yl)-1H-pyrazolo[3,4-b]pyrazin-6-yl)-3-azabicyclo[4.1.0]heptan-7-yl)methanamine
(3-(3-(2-methyl-2H-indazol-4-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-(quinolin-4-yl)-1H-pyrazolo[3,4-b]pyrazin-6-yl)-3-azabicyclo[4.1.0]heptan-7-yl)methanamine
(3-(3-(2-methoxypyridin-3-yl)-1H-pyrazolo[3,4-b]pyrazin-6-yl)-7-(5-methylisoxazol-3-yl)-3-azabicyclo[4.1.0]heptan-7-yl)methanamine
(7-(5-methylisoxazol-3-yl)-3-(3-((S)-6a,7,8,9-tetrahydro-6H-pyrido[3,2-b]pyrrolo[1,2-d][1,4]oxazin-4-yl)-1H-pyrazolo[3,4-b]pyrazin-6-yl)-3-azabicyclo[4.1.0]heptan-7-yl)methanamine
(3-(3-(8-fluoroquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(2,3-dichloropyridin-4-yl)-1H-pyrazolo[3,4-b]pyrazin-6-yl)-7-(5-methylisoxazol-3-yl)-3-azabicyclo[4.1.0]heptan-7-yl)methanamine
5-(6-(7-(aminomethyl)-7-(5-methylisoxazol-3-yl)-3-azabicyclo[4.1.0]heptan-3-yl)-1H-pyrazolo[3,4-b]pyrazin-3-yl)quinoline-8-carbonitrile
(3-(3-(8-chloroquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(8-chloroisoquinolin-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(1,5-naphthyridin-4-yl)-1H-pyrazolo[3,4-b]pyrazin-6-yl)-7-(5-methylisoxazol-3-yl)-3-azabicyclo[4.1.0]heptan-7-yl)methanamine
4-(6-(7-(aminomethyl)-7-(5-methylisoxazol-3-yl)-3-azabicyclo[4.1.0]heptan-3-yl)-1H-pyrazolo[3,4-b]pyrazin-3-yl)-3-chloropyridin-2-amine
((1S,6R,7S)-3-(3-(2,2-difluorobenzo[d][1,3]dioxol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(1,8-naphthyridin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2,3-dihydrobenzo[b][1,4]dioxin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(5-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-chloroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(8-(trifluoromethyl)quinolin-5-yl)-1H-pyrazolo[3,4-b]pyrazin-6-yl)-3-azabicyclo[4.1.0]heptan-7-yl)methanamine
((1S,6R,7S)-3-(3-(3,4-dichloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(7-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-(methoxymethyl)isoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-cyclopropylisoxazol-3-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(3-fluoropyridin-2-yl)-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-(5-fluoropyridin-2-yl)-3-azabicyclo[4.1.0]heptan-7-yl)methanamine
4-(6-((1S,6R,7S)-7-(aminomethyl)-7-(5-methylisoxazol-3-yl)-3-azabicyclo[4.1.0]heptan-3-yl)-1H-pyrazolo[3,4-b]pyrazin-3-yl)-3,3-difluoro-1-methylindolin-2-one
(3-(3-(3-methylbenzo[d]isoxazol-5-yl)-1H-pyrazolo[3,4-b]pyrazin-6-yl)-7-(4-methylisoxazol-3-yl)-3-azabicyclo[4.1.0]heptan-7-yl)methanamine
4-((6-(7-(aminomethyl)-7-(5-methylisoxazol-3-yl)-3-azabicyclo[4.1.0]heptan-3-yl)-1H-pyrazolo[3,4-b]pyrazin-3-yl)thio)-3-chloropyridin-2-amine
(7-(5-methylisoxazol-3-yl)-3-(3-(thieno[3,2-b]pyridin-7-ylthio)-1H-pyrazolo[3,4-b]pyrazin-6-yl)-3-azabicyclo[4.1.0]heptan-7-yl)methanamine
(6-(7-(aminomethyl)-7-(3-fluorophenyl)-3-azabicyclo[4.1.0]heptan-3-yl)-3-(2,3-dichlorophenyl)-1H-pyrazolo[3,4-b]pyrazin-5-yl)methanol
(6-(7-(aminomethyl)-7-(3-fluorophenyl)-3-azabicyclo[4.1.0]heptan-3-yl)-3-phenyl-1H-pyrazolo[3,4-b]pyrazin-5-yl)methanol
(3-((2-amino-3-chloropyridin-4-yl)thio)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol
(3-(2-amino-3-chloropyridin-4-yl)-6-(7-(aminomethyl)-7-(5-methylisoxazol-3-yl)-3-azabicyclo[4.1.0]heptan-3-yl)-1H-pyrazolo[3,4-b]pyrazin-5-yl)methanol
4-((6-(7-(aminomethyl)-7-(2-fluorophenyl)-3-azabicyclo[4.1.0]heptan-3-yl)-5H-pyrrolo[2,3-b]pyrazin-2-yl)thio)-3-chloropyridin-2-amine
(3-(3-(3,4-dihydro-1,5-naphthyridin-1(2H)-yl)-1H-pyrazolo[3,4-b]pyrazin-6-yl)-7-(5-methylisoxazol-3-yl)-3-azabicyclo[4.1.0]heptan-7-yl)methanamine
(3-(7-(aminomethyl)-3-(3-(4-chloro-2-methyl-2H-indazol-5-yl)-1H-pyrazolo[3,4-b]pyrazin-6-yl)-3-azabicyclo[4.1.0]heptan-7-yl)isoxazol-5-yl)methanol
6-(7-(aminomethyl)-7-(5-methylisoxazol-3-yl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one
(7-phenyl-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(thiophen-3-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(pyridin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(pyrimidin-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
(7-(4-methylthiazol-2-yl)-3-(5-((2-(trifluoromethyl)pyridin-3-yl)thio)-1H-imidazo[4,5-b]pyrazin-2-yl)-3-azabicyclo[4.1.0]heptan-7-yl)methanamine
4-((2-(7-(aminomethyl)-7-phenyl-3-azabicyclo[4.1.0]heptan-3-yl)-1H-imidazo[4,5-b]pyrazin-5-yl)thio)-3-chloropyridin-2-amine
4-((2-(7-(aminomethyl)-7-(4-methylthiazol-2-yl)-3-azabicyclo[4.1.0]heptan-3-yl)-1H-imidazo[4,5-b]pyrazin-5-yl)thio)-3-chloropyridin-2-amine
6-(7-(aminomethyl)-7-phenyl-3-azabicyclo[4.1.0]heptan-3-yl)-3-((2-(trifluoromethyl)pyridin-3-yl)thio)pyrazin-2-amine
6-(7-(aminomethyl)-7-(pyridin-2-yl)-3-azabicyclo[4.1.0]heptan-3-yl)-3-((2-(trifluoromethyl)pyridin-3-yl)thio)pyrazin-2-amine
6-(7-(aminomethyl)-7-(4-methylthiazol-2-yl)-3-azabicyclo[4.1.0]heptan-3-yl)-3-((2-(trifluoromethyl)pyridin-3-yl)thio)pyrazin-2-amine
3-((2-amino-3-chloropyridin-4-yl)thio)-6-(7-(aminomethyl)-7-(4-methylthiazol-2-yl)-3-azabicyclo[4.1.0]heptan-3-yl)pyrazin-2-amine
4-((5-(7-(aminomethyl)-7-(4-methylthiazol-2-yl)-3-azabicyclo[4.1.0]heptan-3-yl)imidazo[1,2-c]pyrimidin-8-yl)thio)-3-chloropyridin-2-amine
(3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine
(3-(3-(2,3-dichlorophenyl)-1H-pyrazolo[3,4-b]pyrazin-6-yl)-7-phenyl-3-azabicyclo[4.1.0]heptan-7-yl)methanamine
(3-(3-(4-chloro-2-methyl-2H-indazol-5-yl)-1H-pyrazolo[3,4-b]pyrazin-6-yl)-7-phenyl-3-azabicyclo[4.1.0]heptan-7-yl)methanamine
(3-(3-(2,3-dichlorophenyl)-1H-pyrazolo[3,4-b]pyrazin-6-yl)-7-(pyridin-2-yl)-3-azabicyclo[4.1.0]heptan-7-yl)methanamine
(3-(3-(2,3-dichlorophenyl)-1H-pyrazolo[3,4-b]pyrazin-6-yl)-7-(pyrimidin-2-yl)-3-azabicyclo[4.1.0]heptan-7-yl)methanamine
(3-(3-(2-chlorophenyl)-1H-pyrazolo[3,4-b]pyrazin-6-yl)-7-(4-methylthiazol-2-yl)-3-azabicyclo[4.1.0]heptan-7-yl)methanamine
(3-(3-(3-chlorophenyl)-1H-pyrazolo[3,4-b]pyrazin-6-yl)-7-(4-methylthiazol-2-yl)-3-azabicyclo[4.1.0]heptan-7-yl)methanamine
(3-(3-(2,3-difluorophenyl)-1H-pyrazolo[3,4-b]pyrazin-6-yl)-7-(4-methylthiazol-2-yl)-3-azabicyclo[4.1.0]heptan-7-yl)methanamine
(3-(3-(2-chloro-3-methoxyphenyl)-1H-pyrazolo[3,4-b]pyrazin-6-yl)-7-(4-methylthiazol-2-yl)-3-azabicyclo[4.1.0]heptan-7-yl)methanamine
(7-(4-methylthiazol-2-yl)-3-(3-(quinolin-5-yl)-1H-pyrazolo[3,4-b]pyrazin-6-yl)-3-azabicyclo[4.1.0]heptan-7-yl)methanamine
(7-(4-methylthiazol-2-yl)-3-(3-(naphthalen-1-yl)-1H-pyrazolo[3,4-b]pyrazin-6-yl)-3-azabicyclo[4.1.0]heptan-7-yl)methanamine
(7-(4-methylthiazol-2-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine
(3-(3-(benzo[d][1,3]dioxol-4-yl)-1H-pyrazolo[3,4-b]pyrazin-6-yl)-7-(4-methylthiazol-2-yl)-3-azabicyclo[4.1.0]heptan-7-yl)methanamine
(3-(3-(8-fluoroquinolin-5-yl)-1H-pyrazolo[3,4-b]pyrazin-6-yl)-7-(4-methylthiazol-2-yl)-3-azabicyclo[4.1.0]heptan-7-yl)methanamine
4-((6-(7-(aminomethyl)-7-(4-methylthiazol-2-yl)-3-azabicyclo[4.1.0]heptan-3-yl)-1H-pyrazolo[3,4-b]pyrazin-3-yl)thio)-3-chloropyridin-2-amine
(3-(3-(2,3-dichlorophenyl)-1H-pyrazolo[3,4-b]pyrazin-6-yl)-7-(thiophen-3-yl)-3-azabicyclo[4.1.0]heptan-7-yl)methanamine
(7-(thiophen-3-yl)-3-(3-(2-(trifluoromethyl)pyridin-4-yl)-1H-pyrazolo[3,4-b]pyrazin-6-yl)-3-azabicyclo[4.1.0]heptan-7-yl)methanamine
(7-(thiophen-3-yl)-3-(3-(2-(trifluoromethyl)pyridin-3-yl)-1H-pyrazolo[3,4-b]pyrazin-6-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-pyrazol-5-yl)-3-azabicyclo[4.1.0]heptan-7-yl)methanamine
(3-(3-(2,3-dichlorophenyl)-1H-pyrazolo[3,4-b]pyrazin-6-yl)-7-(1-methyl-1H-pyrazol-3-yl)-3-azabicyclo[4.1.0]heptan-7-yl)methanamine
(3-(3-(2,3-dichlorophenyl)-1H-pyrazolo[3,4-b]pyrazin-6-yl)-7-(isoxazol-3-yl)-3-azabicyclo[4.1.0]heptan-7-yl)methanamine
(6-(7-(aminomethyl)-7-(4-methylthiazol-2-yl)-3-azabicyclo[4.1.0]heptan-3-yl)-3-(quinolin-5-yl)-1H-pyrazolo[3,4-b]pyrazin-5-yl)methanol
(6-(7-(aminomethyl)-7-(4-methylthiazol-2-yl)-3-azabicyclo[4.1.0]heptan-3-yl)-3-(2,3-dichlorophenyl)-1H-pyrazolo[3,4-b]pyrazin-5-yl)methanol
4-(6-(7-(aminomethyl)-7-(4-methylthiazol-2-yl)-3-azabicyclo[4.1.0]heptan-3-yl)-1H-pyrazolo[3,4-b]pyrazin-3-yl)naphthalen-1-yl sulfurofluoridate
6-((1R,5S,6s)-6-(aminomethyl)-6-phenyl-3-azabicyclo[3.1.0]hexan-3-yl)-3-((2-(trifluoromethyl)pyridin-3-yl)thio)pyrazin-2-amine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-amine
((1S,6R,7R)-7-(2,6-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-2,3-dimethyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(4-chloro-3-methoxy-2-methyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
(3-(3-(7-chloro-2-methylbenzo[d]thiazol-6-yl)-5-methyl-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2-chloro-3-(oxazol-2-yl)phenyl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(5-chloro-3-methoxyquinoxalin-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(thieno[2,3-b]pyridin-3-yl)-1H-pyrazolo[3,4-b]pyrazin-6-yl)-3-azabicyclo[4.1.0]heptan-7-yl)methanamine
((1S,6R,7S)-3-(3-(8-fluoroquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(6-methoxyquinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7R)-7-(2,6-difluorophenyl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(2,6-difluorophenyl)-3-azabicyclo[4.1.0]heptan-7-yl)methanamine
((1S,6R,7R)-3-(3-(benzo[d]thiazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(5-chloro-3-methoxyquinoxalin-6-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(3-(4-chloro-2-methyl-2H-indazol-5-yl)-1H-pyrazolo[3,4-b]pyrazin-6-yl)-7-(5-fluoropyridin-2-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(5-fluoropyridin-2-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
((1S,6R,7S)-3-(3-(4-chloro-2-methyl-2H-indazol-5-yl)-1H-pyrazolo[3,4-b]pyrazin-6-yl)-7-(isoxazol-3-yl)-3-azabicyclo[4.1.0]heptan-7-yl)methanamine
((1S,6R,7S)-3-(3-(7-chloro-2-methylbenzo[d]thiazol-6-yl)-1H-pyrazolo[3,4-b]pyrazin-6-yl)-7-(isoxazol-3-yl)-3-azabicyclo[4.1.0]heptan-7-yl)methanamine
((1S,6R,7S)-3-(3-(8-methoxyquinolin-4-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(8-(trifluoromethoxy)quinolin-5-yl)-1H-pyrazolo[3,4-b]pyrazin-6-yl)-3-azabicyclo[4.1.0]heptan-7-yl)methanamine
((1S,6R,7S)-3-(3-(2,4-dimethyl-2H-indazol-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(8-(methoxymethyl)quinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7S)-3-(3-(7-fluoroquinolin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7R)-7-(2-fluorophenyl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-fluoroquinolin-4-yl)-1H-pyrazolo[3,4-b]pyrazin-6-yl)-3-azabicyclo[4.1.0]heptan-7-yl)methanamine
((1S,6R,7S)-3-(3-(8-(difluoromethyl)quinolin-5-yl)-1H-pyrazolo[3,4-b]pyrazin-6-yl)-7-(5-methylisoxazol-3-yl)-3-azabicyclo[4.1.0]heptan-7-yl)methanamine
1-(5-(6-((1S,6R,7S)-7-(aminomethyl)-7-(5-methylisoxazol-3-yl)-3-azabicyclo[4.1.0]heptan-3-yl)-1H-pyrazolo[3,4-b]pyrazin-3-yl)quinolin-8-yl)ethan-1-ol
4-((8-((1S,6R,7R)-7-(aminomethyl)-7-(2-fluorophenyl)-3-azabicyclo[4.1.0]heptan-3-yl)imidazo[1,5-a]pyrazin-5-yl)thio)-3-chloropyridin-2-amine
((1S,6R,7R)-7-(2-fluorophenyl)-3-(3-(pyrazolo[1,5-a]pyridin-5-yl)-1H-pyrazolo[3,4-b]pyrazin-6-yl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)propan-2-ol
((1S,6R,7R)-7-(2-fluorophenyl)-3-(3-(8-isopropoxyquinolin-5-yl)-1H-pyrazolo[3,4-b]pyrazin-6-yl)-3-azabicyclo[4.1.0]heptan-7-yl)methanamine
((1S,6R,7R)-3-(3-(8-cyclopropylquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7S)-7-(3-fluoropyridin-2-yl)-3-(3-(pyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(quinolin-6-yl)-1H-pyrazolo[3,4-b]pyrazin-6-yl)-3-azabicyclo[4.1.0]heptan-7-yl)methanamine
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)ethan-1-ol
((1S,6R,7R)-7-(2-fluorophenyl)-3-(3-(imidazo[1,2-a]pyridin-5-yl)-1H-pyrazolo[3,4-b]pyrazin-6-yl)-3-azabicyclo[4.1.0]heptan-7-yl)methanamine
((1S,6R,7S)-7-(3-fluoropyridin-2-yl)-3-(3-(8-(trifluoromethoxy)quinolin-5-yl)-1H-pyrazolo[3,4-b]pyrazin-6-yl)-3-azabicyclo[4.1.0]heptan-7-yl)methanamine
((1S,6R,7R)-3-(3-(2,4-dimethyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(6-methoxypyrazolo[1,5-a]pyridin-4-yl)-1H-pyrazolo[3,4-b]pyrazin-6-yl)-3-azabicyclo[4.1.0]heptan-7-yl)methanamine
((1S,6R,7S)-3-(3-(1,7-naphthyridin-5-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-3-(3-(2-methoxyquinolin-6-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(2-(trifluoromethyl)quinolin-6-yl)-1H-pyrazolo[3,4-b]pyrazin-6-yl)-3-azabicyclo[4.1.0]heptan-7-yl)methanamine
((1S,6R,7S)-3-(3-(7-methoxyquinolin-3-yl)-1H-pyrazolo[3,4-b]pyrazin-6-yl)-7-(5-methylisoxazol-3-yl)-3-azabicyclo[4.1.0]heptan-7-yl)methanamine
((1S,6R,7S)-7-(5-methylisoxazol-3-yl)-3-(3-(7-(trifluoromethyl)quinolin-3-yl)-1H-pyrazolo[3,4-b]pyrazin-6-yl)-3-azabicyclo[4.1.0]heptan-7-yl)methanamine
((1S,6R,7R)-3-(3-(1,6-naphthyridin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2,3-dihydrofuro[2,3-b]pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1,7-naphthyridin-4-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(7-(8-fluoroquinolin-5-yl)-5H-pyrrolo[2,3-b]pyrazin-3-yl)-7-(5-methylisoxazol-3-yl)-3-azabicyclo[4.1.0]heptan-7-yl)methanamine
((1S,6R,7R)-3-(7-(2,4-dimethyl-2H-indazol-5-yl)-5H-pyrrolo[2,3-b]pyrazin-3-yl)-7-(2-fluorophenyl)-3-azabicyclo[4.1.0]heptan-7-yl)methanamine
((1S,6R,7R)-3-(3-(2-chloro-3-(pyrazin-2-yl)phenyl)-1H-pyrazolo[3,4-b]pyrazin-6-yl)-7-(2-fluorophenyl)-3-azabicyclo[4.1.0]heptan-7-yl)methanamine
(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)methanol
((1S,6R,7R)-3-(3-(8-(difluoromethoxy)quinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(7-methylpyrazolo[1,5-a]pyridin-4-yl)-1H-pyrazolo[3,4-b]pyrazin-6-yl)-3-azabicyclo[4.1.0]heptan-7-yl)methanamine
((1S,6R,7R)-3-(3-(3-bromo-2-chlorophenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)pyrimidin-2-amine
((1S,6R,7R)-3-(3-(8-ethoxyquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(8-(1-methoxyethyl)quinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methoxyquinolin-4-yl)-1H-pyrazolo[3,4-b]pyrazin-6-yl)-3-azabicyclo[4.1.0]heptan-7-yl)methanamine
((1S,6R,7R)-3-(3-(5-chloroquinoxalin-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-3-(oxazol-2-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3,4-dihydro-1,5-naphthyridin-1(2H)-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-chloro-2-(oxazol-2-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(7-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(5-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methylquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-fluoroquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(7-fluoroquinolin-6-yl)-1H-pyrazolo[3,4-b]pyrazin-6-yl)-3-azabicyclo[4.1.0]heptan-7-yl)methanamine
((1S,6R,7R)-3-(3-(3-chloropyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2,3-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-3-chloropyridin-2-amine
((1S,6R,7S)-3-(3-(2,4-dimethyl-2H-indazol-5-yl)-1H-pyrazolo[3,4-b]pyrazin-6-yl)-7-(3-fluoropyridin-2-yl)-3-azabicyclo[4.1.0]heptan-7-yl)methanamine
((1S,6R,7R)-7-(2-chlorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-chlorophenyl)-3-azabicyclo[4.1.0]heptan-3-yl)-1H-pyrazolo[3,4-b]pyrazin-3-yl)-3-chloropyridin-2-amine
((1S,6R,7R)-3-(3-(2,4-dimethyl-2H-indazol-5-yl)-1H-pyrazolo[3,4-b]pyrazin-6-yl)-7-(2-(trifluoromethyl)phenyl)-3-azabicyclo[4.1.0]heptan-7-yl)methanamine
((1S,6R,7R)-7-(2,3-difluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-3-(3-(2,4-dimethyl-2H-indazol-5-yl)-1H-pyrazolo[3,4-b]pyrazin-6-yl)-7-(2-fluoro-3-methylphenyl)-3-azabicyclo[4.1.0]heptan-7-yl)methanamine
((1S,6R,7S)-7-(5-cyclopropyl-3-fluoropyridin-2-yl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(furo[2,3-b]pyridin-4-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
((1S,6R,7R)-7-(2-fluorophenyl)-3-(3-(3-methoxyquinoxalin-6-yl)-1H-pyrazolo[3,4-b]pyrazin-6-yl)-3-azabicyclo[4.1.0]heptan-7-yl)methanamine
((1S,6R,7R)-3-(3-(3-chloro-2-methylpyridin-4-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N,N-dimethylpyridin-2-amine
((1S,6R,7R)-7-(2-fluorophenyl)-3-(3-(7-methoxyquinolin-4-yl)-1H-pyrazolo[3,4-b]pyrazin-6-yl)-3-azabicyclo[4.1.0]heptan-7-yl)methanamine
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-8-yl)-N,N-dimethylmethanamine
((1S,6R,7R)-7-(2-fluorophenyl)-3-(3-(4-methylbenzo[c][1,2,5]oxadiazol-5-yl)-1H-pyrazolo[3,4-b]pyrazin-6-yl)-3-azabicyclo[4.1.0]heptan-7-yl)methanamine
((1S,6R,7R)-3-(3-(4-cyclopropyl-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-fluoro-2-methylpyridin-4-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloropyridin-2-amine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyclopropylpyridin-2-amine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylpyridin-2(1H)-one
((1S,6R,7R)-7-(2,4-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2,4-difluorophenyl)-3-(3-(8-methoxyquinolin-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(8-methoxy-2-methylquinolin-5-yl)-1H-pyrazolo[3,4-b]pyrazin-6-yl)-3-azabicyclo[4.1.0]heptan-7-yl)methanamine
((1S,6R,7R)-3-(3-(4-(difluoromethyl)-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1,4-dimethyl-1H-benzo[d][1,2,3]triazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2,4-dimethyl-2H-benzo[d][1,2,3]triazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,3-dimethylindolin-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-2H-benzo[b][1,4]oxazin-3(4H)-one
((1S,6R,7R)-3-(3-(4-chloro-2-ethyl-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
((1S,6R,7R)-7-(2-fluorophenyl)-3-(3-(8-methyl-[1,2,4]triazolo[4,3-a]pyridin-7-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N,N-dimethylquinoline-8-carboxamide
((1R,5S,6r)-3-(3-(8-fluorodecahydroquinolin-5-yl)-4,7-dihydro-1H-pyrazolo[3,4-b]pyrazin-6-yl)-6-(4-methylthiazol-2-yl)-3-azabicyclo[3.1.0]hexan-6-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methoxypyridin-2-amine
((1S,6R,7R)-3-(3-(2,3-dichloro-4-((1-methyl-1H-pyrazol-3-yl)oxy)phenyl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloroindolin-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylindolin-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoindolin-1-one
((1S,6R,7R)-7-(2-fluorophenyl)-3-(3-(8-methoxyisoquinolin-5-yl)-1H-pyrazolo[3,4-b]pyrazin-6-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinoline-8-carboxamide
((1R,5S,6s)-3-(3-(4-chloro-2-methyl-2H-indazol-5-yl)-1H-pyrazolo[3,4-b]pyrazin-6-yl)-6-(2,6-difluorophenyl)-3-azabicyclo[3.1.0]hexan-6-yl)methanamine
((1S,6R,7R)-7-(2-fluorophenyl)-3-(3-(5-methylquinoxalin-6-yl)-1H-pyrazolo[3,4-b]pyrazin-6-yl)-3-azabicyclo[4.1.0]heptan-7-yl)methanamine
methyl 4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzoate
((1S,6R,7R)-7-(2-fluorophenyl)-3-(3-(imidazo[1,2-a]pyridin-3-yl)-1H-pyrazolo[3,4-b]pyrazin-6-yl)-3-azabicyclo[4.1.0]heptan-7-yl)methanamine
((1S,6R,7R)-3-(3-(7-fluoro-8-methoxyquinolin-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(8-fluoro-2-methylquinolin-5-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-dichlorobenzoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-4-methyl-2H-benzo[b][1,4]oxazin-3(4H)-one
((1S,6R,7R)-3-(3-(4-chloro-2-(oxetan-3-yl)-2H-indazol-5-yl)-1H-pyrazolo[3,4-b]pyrazin-6-yl)-7-(2-fluorophenyl)-3-azabicyclo[4.1.0]heptan-7-yl)methanamine
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dimethyl-1,3-dihydro-2H-benzo[d]imidazol-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-2(1H)-one
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-fluoroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-chloroquinolin-2(1H)-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dihydro-2H-benzo[d]imidazol-2-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-dichlorobenzamide
((1S,6R,7R)-7-(2-fluorophenyl)-3-(3-(quinolin-5-yl)-1H-pyrazolo[3,4-b]pyrazin-6-yl)-3-azabicyclo[4.1.0]heptan-7-yl)methanamine
((1S,6R,7R)-3-(3-(4-chloro-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylisoindolin-1-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzonitrile
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylbenzamide
((1S,6R,7R)-7-(2-fluorophenyl)-3-(3-(isoquinolin-6-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(isoquinolin-7-yl)-1H-pyrazolo[3,4-b]pyrazin-6-yl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)benzo[d]isothiazol-3(2H)-one 1,1-dioxide
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzamide
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroisoquinolin-1(2H)-one
((1S,6R,7R)-7-(2-fluorophenyl)-3-(3-(quinolin-7-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-chloro-5-fluorobenzamide
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)phthalazin-1(2H)-one
((1S,6R,7R)-3-(3-(3-chloroquinolin-7-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-naphthoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinolin-4-amine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methyl-1,3-dihydro-2H-benzo[d]imidazol-2-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4H-pyrido[1,2-a]pyrimidin-4-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3,4-trimethyl-1,3-dihydro-2H-benzo[d]imidazol-2-one
((1S,6R,7R)-7-(2-fluorophenyl)-3-(3-(3-methoxycinnolin-7-yl)-1H-pyrazolo[3,4-b]pyrazin-6-yl)-3-azabicyclo[4.1.0]heptan-7-yl)methanamine
((1S,6R,7R)-3-(3-(3-chloro-2-(oxazol-5-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(oxazol-2-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3-chloro-2-(1-methyl-1H-pyrazol-3-yl)pyridin-4-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)quinoline-8-carboxylic acid
5-(6-((1S,6R,7R)-7-(aminomethyl)-7-(2,3-difluorophenyl)-3-azabicyclo[4.1.0]heptan-3-yl)-1H-pyrazolo[3,4-b]pyrazin-3-yl)-N-methylquinoline-8-carboxamide
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2(1H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one
((1S,6R,7R)-7-(2-fluorophenyl)-3-(3-(spiro[cyclobutane-1,3′-pyrrolo[3,2-b]pyridin]-1′(2′H)-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzoic acid
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methylquinolin-2(1H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-methylbenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-methylisoquinolin-1(2H)-one
((1R,5S,6r)-3-(3-(4-chloro-2-methyl-2H-indazol-5-yl)-1H-pyrazolo[3,4-b]pyrazin-6-yl)-6-(5-methylisoxazol-3-yl)-3-azabicyclo[3.1.0]hexan-6-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzamide
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzoic acid
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-1-methylpyridin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-cyclopropylquinoline-8-carboxamide
((1S,6R,7R)-3-(3-(4-chloro-2-isopropyl-2H-indazol-5-yl)-1H-pyrazolo[3,4-b]pyrazin-6-yl)-7-(2-fluorophenyl)-3-azabicyclo[4.1.0]heptan-7-yl)methanamine
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chlorobenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-N-(methylsulfonyl)quinoline-8-carboxamide
((1R,5S,6r)-3-(3-(2-chloro-4-(1H-pyrazol-1-yl)phenyl)-1H-pyrazolo[3,4-b]pyrazin-6-yl)-6-(4-methylthiazol-2-yl)-3-azabicyclo[3.1.0]hexan-6-yl)methanamine
((1S,6R,7R)-3-(3-(3-chloro-2-(pyrimidin-5-yl)pyridin-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(2H-tetrazol-5-yl)phenyl)-1H-pyrazolo[3,4-b]pyrazin-6-yl)-7-(2-fluorophenyl)-3-azabicyclo[4.1.0]heptan-7-yl)methanamine
7-(aminomethyl)-3-(3-(4-chloro-2-methyl-2H-indazol-5-yl)-1H-pyrazolo[3,4-b]pyrazin-6-yl)-N,N-dimethyl-3-azabicyclo[4.1.0]heptane-7-carboxamide
7-(aminomethyl)-3-(3-(4-chloro-2-methyl-2H-indazol-5-yl)-1H-pyrazolo[3,4-b]pyrazin-6-yl)-N-methyl-3-azabicyclo[4.1.0]heptane-7-carboxamide
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,4,5,6-tetrahydro-7H-pyrrolo[3,4-b]pyridin-7-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methyl-1,3-dihydro-2H-benzo[d]imidazol-2-one
((1S,6R,7R)-7-(2-fluorophenyl)-3-(3-(quinazolin-6-yl)-1H-pyrazolo[3,4-b]pyrazin-6-yl)-3-azabicyclo[4.1.0]heptan-7-yl)methanamine
((1S,6R,7R)-3-(3-(4-chloro-2-methyl-2H-indazol-5-yl)-1H-pyrazolo[3,4-b]pyrazin-6-yl)-7-(3,5-difluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methoxy-1,3-dihydro-2H-benzo[d]imidazol-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinolin-2(1H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)isoquinolin-1(2H)-one
3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-oxadiazol-5-ol
((1S,6R,7R)-3-(3-(2-chloro-4-(1H-pyrazol-5-yl)phenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(2-chloro-4-(1-methyl-1H-pyrazol-4-yl)phenyl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroisoquinolin-1(2H)-one
6-(6-((1S,6R,7R)-7-(aminomethyl)-7-(2-fluorophenyl)-3-azabicyclo[4.1.0]heptan-3-yl)-4H-114-pyrazolo[4,5-b]pyrazin-3-yl)-5-fluoroisoquinolin-1(2H)-one
6-(6-((1S,6R,7R)-7-(aminomethyl)-7-(2-fluorophenyl)-3-azabicyclo[4.1.0]heptan-3-yl)-114,212-pyrazolo[3,4-b]pyrazin-3-yl)-2-methylisoquinolin-1(2H)-one
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)quinazolin-4(3H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1,3-dihydro-2H-benzo[d]imidazol-2-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1,3-dihydro-2H-pyrrolo[2,3-b]pyridin-2-one
1-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)indolin-1-yl)ethan-1-one
((1S,6R,7R)-7-(2-fluorophenyl)-3-(3-(1-methyl-1H-benzo[d]imidazol-6-yl)-1H-pyrazolo[3,4-b]pyrazin-6-yl)-3-azabicyclo[4.1.0]heptan-7-yl)methanamine
6-(6-((1S,6R,7R)-7-(aminomethyl)-7-(3,5-difluorophenyl)-3-azabicyclo[4.1.0]heptan-3-yl)-1H-pyrazolo[3,4-b]pyrazin-3-yl)-5-fluoroisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-methylpicolinamide
((1S,6R,7R)-7-(3,5-difluorophenyl)-3-(3-(2,4-dimethyl-2H-indazol-5-yl)-1H-pyrazolo[3,4-b]pyrazin-6-yl)-3-azabicyclo[4.1.0]heptan-7-yl)methanamine
((1S,6R,7R)-7-(2-fluorophenyl)-3-(3-(2-(trifluoromethyl)quinazolin-6-yl)-1H-pyrazolo[3,4-b]pyrazin-6-yl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyanobenzamide
((1S,6R,7R)-3-(3-(5-chloro-3,4-dihydro-2H-benzo[b][1,4]oxazin-6-yl)-1,7-dihydro-412-pyrazolo[4,3-b]pyrazin-6-yl)-7-(2-fluorophenyl)-3-azabicyclo[4.1.0]heptan-7-yl)methanamine
((1S,6R,7R)-7-(2-fluorophenyl)-3-(3-(indolin-5-yl)-1H-pyrazolo[3,4-b]pyrazin-6-yl)-3-azabicyclo[4.1.0]heptan-7-yl)methanamine
4′-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3′-chloro-3,5-difluoro-2,3,4,5-tetrahydro-[1,1′-biphenyl]-4-ol
5-(6-((1S,6R,7R)-7-(aminomethyl)-7-(2-fluorophenyl)-3-azabicyclo[4.1.0]heptan-3-yl)-4,7-dihydro-1H-pyrazolo[3,4-b]pyrazin-3-yl)-8-fluoroisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-8-fluoro-2-methylisoquinolin-1(2H)-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-fluoro-2-methylisoquinolin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1,3-dimethyl-1,3-dihydro-2H-benzo[d]imidazol-2-one
((1S,6R,7R)-3-(3-(1,4-dimethyl-4,7-dihydro-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-2,4-dihydro-1H-pyrazolo[3,4-b]pyrazin-3-yl)isoquinolin-1-amine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-1-methyl-1,3-dihydro-2H-benzo[d]imidazol-2-one
((1S,6R,7R)-3-(3-(5-chloro-4-methyl-3,4-dihydro-2H-benzo[b][1,4]oxazin-6-yl)-2,3,4,7-tetrahydro-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenol
((1S,6R,7R)-3-(3-(3-chloro-3′,5′-difluoro-[1,1′-biphenyl]-4-yl)-1H-pyrazolo[3,4-b]pyrazin-6-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2H-indazol-2-yl)acetamide
((1S,6R,7R)-3-(3-(4-chloro-1-isopropyl-1H-indazol-5-yl)-1H-pyrazolo[3,4-b]pyrazin-6-yl)-7-(2-fluorophenyl)-3-azabicyclo[4.1.0]heptan-7-yl)methanamine
1-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)pyrrolidin-2-one
((1S,6R,7R)-3-(3-([1,2,4]triazolo[1,5-a]pyridin-6-yl)-1H-pyrazolo[3,4-b]pyrazin-6-yl)-7-(2-fluorophenyl)-3-azabicyclo[4.1.0]heptan-7-yl)methanamine
2-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)acetamide
((1S,6R,7R)-3-(3-(4-chloro-1-methyl-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(3,5-difluorophenyl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-fluorophenol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-fluorophenol
((1S,6R,7R)-3-(3-([1,2,4]triazolo[1,5-a]pyridin-7-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-((2-fluorophenyl)ethynyl)-1H-pyrazolo[3,4-b]pyrazin-6-yl)-3-azabicyclo[4.1.0]heptan-7-yl)methanamine
((1S,6R,7R)-3-(3-(4-chloro-2-(difluoromethyl)-2H-indazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-7H-114-pyrazolo[4,5-b]pyrazin-3-yl)-5-chloro-2-methylisoquinolin-1(2H)-one
((1S,6R,7R)-7-(2-fluorophenyl)-3-(3-(2-methyl-1H-benzo[d]imidazol-5-yl)-1H-pyrazolo[3,4-b]pyrazin-6-yl)-3-azabicyclo[4.1.0]heptan-7-yl)methanamine
((1S,6R,7R)-3-(3-(7-chloro-1-methyl-1H-benzo[d]imidazol-6-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-chloro-2-methylphthalazin-1(2H)-one
6-(6-((1S,6R,7R)-7-(aminomethyl)-7-(2-fluorophenyl)-3-azabicyclo[4.1.0]heptan-3-yl)-114,212-pyrazolo[3,4-b]pyrazin-3-yl)-5-chlorophthalazin-1(2H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzo[d]oxazol-2(3H)-one
((1S,6R,7R)-3-(5-((3-chloro-2-(1H-pyrazol-1-yl)pyridin-4-yl)thio)-1H-imidazo[4,5-b]pyrazin-2-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-5-methyl-4-oxo-4,5-dihydro-1H-pyrazolo[3,4-d]pyrimidin-3-yl)quinoline-8-carboxamide
6-((1S,6R,7R)-7-(aminomethyl)-7-(3,5-difluorophenyl)-3-azabicyclo[4.1.0]heptan-3-yl)-3-(4-chloro-2-methyl-2H-indazol-5-yl)-5-methyl-1,5-dihydro-4H-pyrazolo[3,4-d]pyrimidin-4-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-(trifluoromethyl)pyridin-2-ol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorobenzenesulfonamide
((1S,6R,7R)-7-(2-fluorophenyl)-3-(3-(1-methyl-1H-pyrazol-4-yl)-1H-pyrazolo[3,4-b]pyrazin-6-yl)-3-azabicyclo[4.1.0]heptan-7-yl)methanamine
((1S,6R,7R)-3-(3-(1,5-dimethyl-1H-pyrazol-4-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(4-chloro-1,2-dimethyl-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2-methylisoindolin-1-one
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-5-fluoro-2-(2-hydroxyethyl)isoquinolin-1(2H)-one
((1S,6R,7R)-7-(2-fluorophenyl)-3-(3-(1-methoxyisoquinolin-6-yl)-2,4-dihydro-1H-pyrazolo[3,4-b]pyrazin-6-yl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-methylbenzo[d]oxazol-2(3H)-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,5-difluorophenol
3-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenol
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloro-N-cyano-N-methylbenzamide
6-(6-((1S,6R,7R)-7-(aminomethyl)-7-(2-fluorophenyl)-3-azabicyclo[4.1.0]heptan-3-yl)-114,712-pyrazolo[5,4-b]pyrazin-3-yl)-5-fluoro-2-(2-methoxyethyl)isoquinolin-1(2H)-one
((1S,6R,7R)-3-(3-(4-chloro-2,3-dimethyl-2,3-dihydro-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenyl acetate
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2-(trifluoromethyl)phenol
((1S,6R,7R)-3-(3-(4-chloro-2-(2,2,2-trifluoroethyl)-2H-indazol-5-yl)-1H-pyrazolo[3,4-b]pyrazin-6-yl)-7-(2-fluorophenyl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-3,3-difluoro-1-methylindolin-2-one
((1S,6R,7R)-3-(3-(7-chloro-2-methyl-2H-indazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(7-chloro-1-methyl-1H-indazol-6-yl)-1H-pyrazolo[3,4-b]pyrazin-6-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-2,4-dihydro-1H-pyrazolo[3,4-b]pyrazin-3-yl)-4-chloro-2H-indazol-2-yl)ethan-1-ol
((1S,6R,7R)-3-(3-(3,3-dimethylbut-1-yn-1-yl)-1H-pyrazolo[3,4-b]pyrazin-6-yl)-7-(2-fluorophenyl)-3-azabicyclo[4.1.0]heptan-7-yl)methanamine
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-fluorobenzamide
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-methylpyridin-2-amine
((1S,6R,7R)-3-(3-(4-chloro-2-methyl-2H-indazol-5-yl)-1H-pyrazolo[3,4-b]pyrazin-6-yl)-7-(2,5-difluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-7-chloroisoquinolin-1(2H)-one
((1S,6R,7R)-7-(2-fluorophenyl)-3-(3-(1,3,5-trimethyl-1H-pyrazol-4-yl)-1H-pyrazolo[3,4-b]pyrazin-6-yl)-3-azabicyclo[4.1.0]heptan-7-yl)methanamine
7-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3,4-dihydroquinazolin-2(1H)-one
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-3,3-difluoroindolin-2-one
4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,3-difluorophenol
5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-6-chloroindoline-2,3-dione
((1S,6R,7R)-7-(2-fluorophenyl)-3-(3-(8-fluoroquinolin-5-yl)-1H-pyrazolo[3,4-b]pyrazin-6-yl)-3-azabicyclo[4.1.0]heptan-7-yl)methanamine
((1S,6R,7R)-3-(3-(2-chloro-4-(5-methyl-1,2,4-oxadiazol-3-yl)phenyl)-1H-pyrazolo[3,4-b]pyrazin-6-yl)-7-(2-fluorophenyl)-3-azabicyclo[4.1.0]heptan-7-yl)methanamine
2-((1S,6R,7R)-7-(aminomethyl)-7-(2-fluorophenyl)-3-azabicyclo[4.1.0]heptan-3-yl)-5-(4-chloro-2-methyl-1H-214-indazol-5-yl)-3-methyl-3,7-dihydro-4H-pyrrolo[2,3-d]pyrimidin-4-one
3-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chlorophenyl)-1,2,4-thiadiazol-5-ol
6-(2-((1S,6R,7R)-7-(aminomethyl)-7-(2-fluorophenyl)-3-azabicyclo[4.1.0]heptan-3-yl)-3-methyl-4-oxo-4,7-dihydro-3H-pyrrolo[2,3-d]pyrimidin-5-yl)-5-fluoroisoquinolin-1(2H)-one
(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-2,6-difluorophenyl)methanol
((1S,6R,7R)-7-(2-fluorophenyl)-3-(5-(quinolin-4-yl)-5H-pyrrolo[2,3-b]pyrazin-2-yl)-3-azabicyclo[4.1.0]heptan-7-yl)methanamine
N-(((1S,6R,7R)-7-(2-fluorophenyl)-3-(3-(2-methyl-2H-indazol-5-yl)-1H-pyrazolo[3,4-b]pyrazin-6-yl)-3-azabicyclo[4.1.0]heptan-7-yl)methyl)acetamide
3-((2-amino-3-chloropyridin-4-yl)thio)-6-((1S,6R,7R)-7-(aminomethyl)-7-(2-fluorophenyl)-3-azabicyclo[4.1.0]heptan-3-yl)pyrazin-2-amine
4-((6-((1S,6R,7R)-7-(aminomethyl)-7-(2-fluorophenyl)-3-azabicyclo[4.1.0]heptan-3-yl)pyrido[2,3-b]pyrazin-2-yl)thio)-3-chloropyridin-2-amine
((S)-1-(4-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-3-chloropyridin-2-yl)pyrrolidin-3-yl)methanol
4-((6-((1R,6S,7S)-7-(aminomethyl)-7-(2-fluorophenyl)-3-azabicyclo[4.1.0]heptan-3-yl)-1,5-naphthyridin-2-yl)thio)-3-chloropyridin-2-amine
((1S,6R,7R)-3-(5-((3-chloro-2-(1H-pyrazol-1-yl)-1,4-dihydropyridin-4-yl)thio)pyrazin-2-yl)-7-(2-fluorophenyl)-3-azabicyclo[4.1.0]heptan-7-yl)methanamine
((1S,6R,7R)-3-(3-(2-(difluoromethyl)-4-fluoro-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(3-(1-phenyl-1H-pyrazol-4-yl)-1H-pyrazolo[3,4-b]pyrazin-6-yl)-3-azabicyclo[4.1.0]heptan-7-yl)methanamine
((1S,6R,7R)-3-(3-(4-chloro-1,4-dihydrobenzo[c][1,2,5]thiadiazol-5-yl)-1H-pyrazolo[3,4-b]pyrazin-6-yl)-7-(2-fluorophenyl)-3-azabicyclo[4.1.0]heptan-7-yl)methanamine
6-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-7-chlorobenzo[d]thiazol-2(3H)-one
((1S,6R,7R)-3-(3-(4-chloro-2-(2-methoxyethyl)-2H-indazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-7-(2-fluorophenyl)-3-(6-(2-methyl-2H-indazol-5-yl)-1,5-naphthyridin-2-yl)-3-azabicyclo[4.1.0]heptan-7-yl)methanamine
4-((2-((1S,6R,7R)-7-(aminomethyl)-7-(2-fluorophenyl)-3-azabicyclo[4.1.0]heptan-3-yl)pyrido[2,3-b]pyrazin-6-yl)thio)-3-chloropyridin-2-amine
((1S,6R,7R)-3-(3-(7-chloro-1-methyl-1H-benzo[d]imidazol-6-yl)-1H-pyrazolo[3,4-b]pyrazin-6-yl)-7-(2,5-difluorophenyl)-3-azabicyclo[4.1.0]heptan-7-yl)methanamine
4-((6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)thio)-3-chloropyridin-2-amine
((1S,6R,7R)-3-(6-((3-chloro-2-(1H-pyrazol-1-yl)pyridin-4-yl)thio)pyrido[2,3-b]pyrazin-2-yl)-7-(2-fluorophenyl)-3-azabicyclo[4.1.0]heptan-7-yl)methanamine
((1S,6R,7R)-3-(3-(3-fluoro-4-methoxyphenyl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-((4-chloro-2-methyl-2H-indazol-5-yl)thio)pyrido[2,3-b]pyrazin-2-yl)-7-(2-fluorophenyl)-3-azabicyclo[4.1.0]heptan-7-yl)methanamine
4-((5-((1S,6R,7R)-7-(aminomethyl)-7-(2-fluorophenyl)-3-azabicyclo[4.1.0]heptan-3-yl)pyrazin-2-yl)thio)-3-chloropyridin-2-amine
((1S,6R,7R)-3-(5-((3-chloro-2-(1H-pyrazol-1-yl)pyridin-4-yl)thio)pyrazin-2-yl)-7-(2,5-difluorophenyl)-3-azabicyclo[4.1.0]heptan-7-yl)methanamine
((1R,7S)-4-(5-((3-chloro-2-(1H-pyrazol-1-yl)pyridin-4-yl)thio)pyrazin-2-yl)-8-(2-fluorophenyl)-4-azabicyclo[5.1.0]octan-8-yl)methanamine
((1R,7S)-4-(3-(4-chloro-2-methyl-2H-indazol-5-yl)-1H-pyrazolo[3,4-b]pyrazin-6-yl)-8-(2-fluorophenyl)-4-azabicyclo[5.1.0]octan-8-yl)methanamine
((1S,6R,7R)-3-(3-(4-chloro-1H-indol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-(3,4-dihydro-1,5-naphthyridin-1(2H)-yl)pyrido[2,3-b]pyrazin-2-yl)-7-(2-fluorophenyl)-3-azabicyclo[4.1.0]heptan-7-yl)methanamine
((1S,6R,7R)-7-(2-fluorophenyl)-3-(6-(pyridin-4-ylthio)-3,4-dihydropyrido[2,3-b]pyrazin-2-yl)-3-azabicyclo[4.1.0]heptan-7-yl)methanamine
2-((1S,6R,7R)-7-(aminomethyl)-7-(2-fluorophenyl)-3-azabicyclo[4.1.0]heptan-3-yl)-7-(2,3-dichlorophenyl)-4H-pyrido[1,2-a]pyrimidin-4-one
((1S,6R,7R)-3-(6-(4-fluorophenoxy)pyrido[2,3-b]pyrazin-2-yl)-7-(2-fluorophenyl)-3-azabicyclo[4.1.0]heptan-7-yl)methanamine
7-((2-amino-3-chloropyridin-4-yl)thio)-2-((1S,6R,7R)-7-(aminomethyl)-7-(2-fluorophenyl)-3-azabicyclo[4.1.0]heptan-3-yl)-4H-pyrido[1,2-a]pyrimidin-4-one
6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one
6-((1S,6R,7R)-7-(aminomethyl)-7-(2,5-difluorophenyl)-3-azabicyclo[4.1.0]heptan-3-yl)-3-(2,3-dichlorophenyl)-2-methylpyrimidin-4(3H)-one
((1S,6R,7R)-3-(3-(4-chloro-1-cyclopropyl-1H-indazol-5-yl)-1H-pyrazolo[3,4-b]pyrazin-6-yl)-7-(2-fluorophenyl)-3-azabicyclo[4.1.0]heptan-7-yl)methanamine
3-((2-amino-3-chloropyridin-4-yl)thio)-6-((1S,6R,7R)-7-(aminomethyl)-7-(2,5-difluorophenyl)-3-azabicyclo[4.1.0]heptan-3-yl)pyrazin-2-amine
((1S,6R,7R)-7-(2-fluorophenyl)-3-(6-(2-methyl-2H-indazol-5-yl)pyrido[2,3-b]pyrazin-2-yl)-3-azabicyclo[4.1.0]heptan-7-yl)methanamine
((1S,6R,7R)-3-(3-(2,3-dichlorophenyl)imidazo[1,5-a]pyrazin-8-yl)-7-(2-fluorophenyl)-3-azabicyclo[4.1.0]heptan-7-yl)methanamine
((1S,6R,7R)-3-(3-(1-(difluoromethyl)-1H-benzo[d]imidazol-6-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(3-(1-(difluoromethyl)-1H-benzo[d]imidazol-5-yl)-1H-pyrazolo[3,4-b]pyrazin-6-yl)-7-(2-fluorophenyl)-3-azabicyclo[4.1.0]heptan-7-yl)methanamine
((1S,6R,7R)-3-(6-((3-fluoro-2-methylpyridin-4-yl)thio)pyrido[2,3-b]pyrazin-2-yl)-7-(2-fluorophenyl)-3-azabicyclo[4.1.0]heptan-7-yl)methanamine
((1S,6R,7R)-3-(3-(4-chloro-2-cyclopropyl-2H-indazol-5-yl)-1H-pyrazolo[3,4-b]pyrazin-6-yl)-7-(2-fluorophenyl)-3-azabicyclo[4.1.0]heptan-7-yl)methanamine
4-((5-((1S,6R,7R)-7-(aminomethyl)-7-(2-fluorophenyl)-3-azabicyclo[4.1.0]heptan-3-yl)pyrazin-2-yl)thio)-3-chlorobenzamide
((1S,6R,7R)-7-(2-fluorophenyl)-3-(5-(quinolin-5-ylthio)pyrazin-2-yl)-3-azabicyclo[4.1.0]heptan-7-yl)methanamine
((1S,6R,7R)-3-(5-((3-fluoro-2-methylpyridin-4-yl)thio)pyrazin-2-yl)-7-(2-fluorophenyl)-3-azabicyclo[4.1.0]heptan-7-yl)methanamine
((1S,6R,7R)-7-(2,5-difluorophenyl)-3-(5-((3-fluoro-2-methylpyridin-4-yl)thio)pyrazin-2-yl)-3-azabicyclo[4.1.0]heptan-7-yl)methanamine
((1S,6R,7R)-3-(6-(2,4-dimethyl-2H-indazol-5-yl)pyrido[2,3-b]pyrazin-2-yl)-7-(2-fluorophenyl)-3-azabicyclo[4.1.0]heptan-7-yl)methanamine
((1S,6R,7R)-7-(2,5-difluorophenyl)-3-(6-((3-fluoro-2-methylpyridin-4-yl)thio)pyrido[2,3-b]pyrazin-2-yl)-3-azabicyclo[4.1.0]heptan-7-yl)methanamine
((1S,6R,7R)-3-(5-((4-chloro-2-methyl-2H-indazol-5-yl)thio)pyrazin-2-yl)-7-(2-fluorophenyl)-3-azabicyclo[4.1.0]heptan-7-yl)methanamine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-2H-indazol-2-yl)-N,N-dimethylacetamide
3-((2-amino-3-chloropyridin-4-yl)thio)-6-((1S,6R,7R)-7-(aminomethyl)-7-(2,3-difluorophenyl)-3-azabicyclo[4.1.0]heptan-3-yl)pyrazin-2-amine
6-((1S,6R,7R)-7-(aminomethyl)-7-(2,5-difluorophenyl)-3-azabicyclo[4.1.0]heptan-3-yl)-3-((3-chloro-2-cyclopropylpyridin-4-yl)thio)pyrazin-2-amine
6-((1S,6R,7R)-7-(aminomethyl)-7-(2,5-difluorophenyl)-3-azabicyclo[4.1.0]heptan-3-yl)-3-((3-fluoro-2-methylpyridin-4-yl)thio)pyrazin-2-amine
2-(5-(6-((1S,6R,7R)-7-(aminomethyl)-7-(2-fluorophenyl)-3-azabicyclo[4.1.0]heptan-3-yl)-1H-pyrazolo[3,4-b]pyrazin-3-yl)-4-chloro-1H-indazol-1-yl)-N,N-dimethylacetamide
6-((1R,5S,6r)-6-(aminomethyl)-6-(4-methylthiazol-2-yl)-3-azabicyclo[3.1.0]hexan-3-yl)-3-(2,3-dichlorophenyl)-2-methylpyrimidin-4(3H)-one
3-((2-amino-3-chloropyridin-4-yl)thio)-6-((1R,5S,6r)-6-(aminomethyl)-6-(4-methylthiazol-2-yl)-3-azabicyclo[3.1.0]hexan-3-yl)pyrazin-2-amine
((1S,6R,7R)-7-(2-fluorophenyl)-3-(6-(8-methoxyquinolin-6-yl)pyrido[2,3-b]pyrazin-2-yl)-3-azabicyclo[4.1.0]heptan-7-yl)methanamine
((1S,6R,7R)-7-(2-fluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine
((1R,6S,7R)-3-(3-(2,3-dichlorophenyl)-1H-pyrazolo[3,4-b]pyrazin-6-yl)-7-(4-methylthiazol-2-yl)-3-azabicyclo[4.1.0]heptan-7-yl)methanamine
((1S,6R,7R)-3-(3-(4-chloropyrazolo[1,5-a]pyridin-5-yl)-1H-pyrazolo[3,4-b]pyrazin-6-yl)-7-(2-fluorophenyl)-3-azabicyclo[4.1.0]heptan-7-yl)methanamine
((1S,6R,7S)-3-(3-(4-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(4-chloro-2-methylpyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
6-(6-((1R,5S,6r)-6-(aminomethyl)-6-(4-methylthiazol-2-yl)-3-azabicyclo[3.1.0]hexan-3-yl)-4,7-dihydro-1H-pyrazolo[3,4-b]pyrazin-3-yl)-5-fluoroisoquinolin-1(2H)-one
(3-(3-(8-chloroquinolin-5-yl)-1H-pyrazolo[3,4-b]pyrazin-6-yl)-7-(4-methylthiazol-2-yl)-3-azabicyclo[4.1.0]heptan-7-yl)methanamine
4-(6-(7-(aminomethyl)-7-(4-methyl-2,3-dihydrothiazol-2-yl)-3-azabicyclo[4.1.0]heptan-3-yl)-4,7-dihydro-1H-pyrazolo[3,4-b]pyrazin-3-yl)naphthalene-1-sulfonyl fluoride
((1S,6R,7S)-3-(3-(7-chloro-2-methylbenzo[d]thiazol-6-yl)-1H-pyrazolo[3,4-b]pyrazin-6-yl)-7-(4-methylthiazol-2-yl)-3-azabicyclo[4.1.0]heptan-7-yl)methanamine
((1S,6R,7S)-3-(3-(3,4-dichloro-2-methyl-2H-indazol-5-yl)-1H-pyrazolo[3,4-b]pyrazin-6-yl)-7-(4-methylthiazol-2-yl)-3-azabicyclo[4.1.0]heptan-7-yl)methanamine
(7-(4-chlorothiazol-2-yl)-3-(3-(8-fluoroquinolin-5-yl)-4H-114-pyrazolo[4,5-b]pyrazin-6-yl)-3-azabicyclo[4.1.0]heptan-7-yl)methanamine
7-((5-((1R,5S,6r)-6-(aminomethyl)-6-(4-methylthiazol-2-yl)-3-azabicyclo[3.1.0]hexan-3-yl)pyrazin-2-yl)thio)-8-chloro-N,N-dimethylimidazo[1,2-a]pyridine-2-carboxamide
5-(6-((1S,6R,7S)-7-(aminomethyl)-7-(4-methylthiazol-2-yl)-3-azabicyclo[4.1.0]heptan-3-yl)-2,4-dihydro-1H-pyrazolo[3,4-b]pyrazin-3-yl)quinolin-8-yl sulfurofluoridate
5-(6-((1S,6R,7R)-7-(aminomethyl)-7-(2-fluorophenyl)-3-azabicyclo[4.1.0]heptan-3-yl)-1,4-dihydro-712-pyrazolo[3,4-b]pyrazin-3-yl)quinolin-8-yl sulfurofluoridate
4-(6-(7-(aminomethyl)-7-(4-methyl-2H-114-thiazol-2-yl)-3-azabicyclo[4.1.0]heptan-3-yl)-1H-pyrazolo[3,4-b]pyrazin-3-yl)-5,6-dichlorocyclohexa-1,4-dien-1-yl sulfurofluoridate
((1S,6R,7R)-3-(3-(3-chloro-2-(1H-pyrazol-1-yl)pyridin-4-yl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(2,3-dichlorophenyl)-1H-pyrazolo[3,4-b]pyrazin-6-yl)-6-phenyl-3-azabicyclo[3.1.0]hexan-6-yl)methanamine
((1R,5S,6s)-3-(3-(4-chloro-2-methyl-2H-indazol-5-yl)-1H-pyrazolo[3,4-b]pyrazin-6-yl)-6-phenyl-3-azabicyclo[3.1.0]hexan-6-yl)methanamine
((1S,6R,7R)-3-(3-(2-chloro-4-(2H-114-pyrrol-1-yl)phenyl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(8-fluoroquinolin-6-yl)-1,7-dihydro-412-pyrazolo[4,3-b]pyrazin-6-yl)-6-phenyl-3-azabicyclo[3.1.0]hexan-6-yl)methanamine
((1R,5S,6s)-3-(3-(5-chloroquinoxalin-6-yl)-1H-pyrazolo[3,4-b]pyrazin-6-yl)-6-phenyl-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloropyrazolo[1,5-a]pyridin-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(8-fluorooctahydro-2H-112-quinolin-4-yl)-6,7-dihydro-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(5-chloroquinoxalin-6-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(7-chloro-2-methylbenzo[d]thiazol-6-yl)-1,7-dihydro-412-pyrazolo[4,3-b]pyrazin-6-yl)-6-(4-methylthiazol-2-yl)-3-azabicyclo[3.1.0]hexan-6-yl)methanamine
6-(6-((1R,5S,6r)-6-(aminomethyl)-6-((S)-4-methyl-2,3-dihydrothiazol-2-yl)-3-azabicyclo[3.1.0]hexan-3-yl)-3,7-dihydro-114,212-pyrazolo[3,4-b]pyrazin-3-yl)isoquinolin-1(2H)-one
((1R,5S,6r)-3-(3-(4-chlorobenzo[c]isothiazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(7-chloro-2-(difluoromethyl)-4H-214-indazol-6-yl)-2,3-dihydro-1H-pyrazolo[3,4-b]pyrazin-6-yl)-6-((R)-4-methyl-312-thiazol-2-yl)-3-azabicyclo[3.1.0]hexan-6-yl)methanamine
((1R,5S,6r)-3-(3-(2-(difluoromethyl)-4-fluoro-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-2-(difluoromethyl)-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chlorobenzo[c][1,2,5]thiadiazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-2-(difluoromethyl)-4,7-dihydro-2H-indazol-5-yl)-1H-pyrazolo[3,4-b]pyrazin-6-yl)-6-(isothiazol-3-yl)-3-azabicyclo[3.1.0]hexan-6-yl)methanamine
6-((1R,5S,6r)-6-(aminomethyl)-6-(4-methylthiazol-2-yl)-3-azabicyclo[3.1.0]hexan-3-yl)-3-(7-chloro-2-methylbenzo[d]thiazol-6-yl)-5-methyl-1,5-dihydro-4H-pyrazolo[3,4-d]pyrimidin-4-one
6-((1R,5S,6r)-6-(aminomethyl)-6-(4-chlorothiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
6-((1R,5S,6r)-6-(aminomethyl)-6-(4-(trifluoromethyl)thiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
6-((1R,5S,6r)-6-(aminomethyl)-6-(4-methylthiazol-2-yl)-3-azabicyclo[3.1.0]hexan-3-yl)-3-(4-chloro-2-methyl-2H-indazol-5-yl)-5-methyl-1,5-dihydro-4H-pyrazolo[3,4-d]pyrimidin-4-one
2-((1R,5S,6r)-6-(aminomethyl)-6-(4-methylthiazol-2-yl)-3-azabicyclo[3.1.0]hexan-3-yl)-5-(4-chloro-2-methyl-2H-indazol-5-yl)-3-methyl-3,7-dihydro-4H-pyrrolo[2,3-d]pyrimidin-4-one
((1R,5S,6r)-3-(3-(4-chloro-2-methyl-2H-indazol-5-yl)-1H-pyrazolo[3,4-b]pyrazin-6-yl)-6-(1-methyl-1H-pyrazol-3-yl)-3-azabicyclo[3.1.0]hexan-6-yl)methanamine
((1R,5S,6r)-3-(3-(7-chloro-2-methylbenzo[d]thiazol-6-yl)-1H-pyrazolo[3,4-b]pyrazin-6-yl)-6-(1-methyl-1H-pyrazol-3-yl)-3-azabicyclo[3.1.0]hexan-6-yl)methanamine
((1R,5S,6r)-3-(3-(3-chloro-2-methoxypyridin-4-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(3-fluoro-2-methyl-1,2-dihydropyridin-4-yl)-1H-pyrazolo[3,4-b]pyrazin-6-yl)-6-(4-methylthiazol-2-yl)-3-azabicyclo[3.1.0]hexan-6-yl)methanamine
2-(5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-4-chloro-2H-indazol-2-yl)acetonitrile
((1R,5S,6r)-3-(3-(7-chlorobenzo[d][1,2,3]thiadiazol-6-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-1-methyl-1H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7S)-3-(3-(6-chloropyrazolo[1,5-a]pyridin-5-yl)-1H-pyrazolo[3,4-b]pyrazin-6-yl)-7-(4-methylthiazol-2-yl)-3-azabicyclo[4.1.0]heptan-7-yl)methanamine
((1R,5S,6r)-6-(4-methylthiazol-2-yl)-3-(3-(pyrazolo[1,5-a]pyridin-4-yl)-1H-pyrazolo[3,4-b]pyrazin-6-yl)-3-azabicyclo[3.1.0]hexan-6-yl)methanamine
((1R,5S,6r)-3-(3-(7-chloro-1-methyl-1H-benzo[d]imidazol-6-yl)-1H-pyrazolo[3,4-b]pyrazin-6-yl)-6-(4-methylthiazol-2-yl)-3-azabicyclo[3.1.0]hexan-6-yl)methanamine
1-((1R,5S,6r)-3-(3-(4-chloro-2-methyl-2H-indazol-5-yl)-1H-pyrazolo[3,4-b]pyrazin-6-yl)-6-(4-methylthiazol-2-yl)-3-azabicyclo[3.1.0]hexan-6-yl)-N-methylmethanamine
((1R,5S,6r)-3-(3-(4-chloro-2-methyl-2H-indazol-5-yl)-1H-pyrazolo[3,4-b]pyrazin-6-yl)-6-(2-methylthiazol-4-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(thiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-chlorothiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(4-(trifluoromethyl)thiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(5-fluorothiazol-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(1,2,5-thiadiazol-3-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(pyridin-2-yl)-3-azabicyclo[3.1.0]hexan-6-yl)methanamine
((1R,5S,6r)-3-(3-(7-chloro-2-methylbenzo[d]thiazol-6-yl)-1H-pyrazolo[3,4-b]pyrazin-6-yl)-6-(pyridin-2-yl)-3-azabicyclo[3.1.0]hexan-6-yl)methanamine
((1R,5S,6r)-3-(3-(4-chloro-2-methyl-3H-214-indazol-5-yl)-1H-pyrazolo[3,4-b]pyrazin-6-yl)-6-(5-methyl-1,4-dihydropyridin-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(6-chloropyridin-2-yl)-3-azabicyclo[3.1.0]hexan-6-yl)methanamine
((1R,5S,6r)-3-(3-(4-chloro-2-methyl-2H-indazol-5-yl)-1H-pyrazolo[3,4-b]pyrazin-6-yl)-6-(5-fluoropyridin-2-yl)-3-azabicyclo[3.1.0]hexan-6-yl)methanamine
((1R,5S,6r)-3-(3-(4-chloro-2-methyl-3,7-dihydro-2H-indazol-5-yl)-1H-pyrazolo[3,4-b]pyrazin-6-yl)-6-(6-methyl-1,6-dihydropyridin-2-yl)-3-azabicyclo[3.1.0]hexan-6-yl)methanamine
4-((2-((1R,5S,6r)-6-(aminomethyl)-6-(4-methylthiazol-2-yl)-3-azabicyclo[3.1.0]hexan-3-yl)pyrido[2,3-b]pyrazin-6-yl)thio)-3-chloropyridin-2-amine
4-((5-((1R,5S,6r)-6-(aminomethyl)-6-(4-methylthiazol-2-yl)-3-azabicyclo[3.1.0]hexan-3-yl)-1,4-dihydropyrazin-2-yl)thio)-3-chloropyridin-2-amine
((1R,5S,6s)-3-(3-(4-chloro-2-methyl-2H-indazol-5-yl)-1H-pyrazolo[3,4-b]pyrazin-6-yl)-6-(2-fluorophenyl)-3-azabicyclo[3.1.0]hexan-6-yl)methanamine
((1S,6R,7R)-3-(3-(3-chloro-2-methoxypyridin-4-yl)-1H-pyrazolo[3,4-b]pyrazin-6-yl)-7-(2-fluorophenyl)-3-azabicyclo[4.1.0]heptan-7-yl)methanamine
((1R,5S,6r)-3-(3-(4-chloro-2-methyl-2H-indazol-5-yl)-1H-pyrazolo[3,4-b]pyrazin-6-yl)-6-(5-methyl-1,3,4-thiadiazol-2-yl)-3-azabicyclo[3.1.0]hexan-6-yl)methanamine
((1S,6R,7S)-3-(7-(8-fluoroquinolin-5-yl)-5H-pyrrolo[2,3-b]pyrazin-3-yl)-7-((R)-4-methyl-2,3-dihydrothiazol-2-yl)-3-azabicyclo[4.1.0]heptan-7-yl)methanamine
5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-1,3-dimethyl-1,3,4,7-tetrahydro-2H-benzo[d]imidazol-2-one
5-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)quinoline-8-carboxamide
((1R,5S,6r)-3-(3-(4-chloro-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(3-(4-chloro-1-(difluoromethyl)-1H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7R)-7-(2-fluorophenyl)-3-(7-(8-fluoroquinolin-5-yl)-5H-pyrrolo[2,3-b]pyrazin-3-yl)-3-azabicyclo[4.1.0]heptan-7-yl)methanamine
5-(6-((1S,6R,7R)-7-(aminomethyl)-7-(2-fluorophenyl)-3-azabicyclo[4.1.0]heptan-3-yl)-4,7-dihydro-1H-pyrazolo[3,4-b]pyrazin-3-yl)quinoline-8-sulfonyl fluoride
((1S,6R,7R)-3-(3-(3-chloro-2-fluoropyridin-4-yl)-1H-pyrazolo[3,4-b]pyrazin-6-yl)-7-(2-fluorophenyl)-3-azabicyclo[4.1.0]heptan-7-yl)methanamine
((1R,5S,6s)-3-(3-(8-fluoroquinolin-5-yl)-1H-pyrazolo[3,4-b]pyrazin-6-yl)-6-phenyl-3-azabicyclo[3.1.0]hexan-6-yl)methanamine
((1R,5S,6r)-3-(3-(5-chloroquinoxalin-6-yl)-1H-pyrazolo[3,4-b]pyrazin-6-yl)-6-(1-methyl-1H-pyrazol-3-yl)-3-azabicyclo[3.1.0]hexan-6-yl)methanamine
4-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-3-chlorobenzamide
5-(6-((1R,5S,6r)-6-(aminomethyl)-6-((R)-4-methyl-2,3-dihydrothiazol-2-yl)-3-azabicyclo[3.1.0]hexan-3-yl)-1H-pyrazolo[3,4-b]pyrazin-3-yl)-1-methylindolin-2-one
((1R,5S,6r)-3-(3-(4-chloro-2-methyl-3H-214-indazol-5-yl)-1H-pyrazolo[3,4-b]pyrazin-6-yl)-6-(2-methyloxazol-4-yl)-3-azabicyclo[3.1.0]hexan-6-yl)methanamine
((1R,5S,6r)-3-(6-((3-chloro-2-(1H-pyrazol-1-yl)pyridin-4-yl)thio)pyrido[2,3-b]pyrazin-2-yl)-6-(4-methylthiazol-2-yl)-3-azabicyclo[3.1.0]hexan-6-yl)methanamine
((1R,5S,6r)-3-(3-(3-chloro-2-(1H-pyrazol-1-yl)pyridin-4-yl)-1H-pyrazolo[3,4-b]pyrazin-6-yl)-6-(4-methylthiazol-2-yl)-3-azabicyclo[3.1.0]hexan-6-yl)methanamine
4-(6-((1R,5S,6r)-6-(aminomethyl)-6-(4-methylthiazol-2-yl)-3-azabicyclo[3.1.0]hexan-3-yl)-1H-pyrazolo[3,4-b]pyrazin-3-yl)-3-chloropyridin-2-amine
((1R,5S,6r)-3-(3-(4-chloro-2-(2,2,2-trifluoroethyl)-2H-indazol-5-yl)-1,7-dihydro-412-pyrazolo[4,3-b]pyrazin-6-yl)-6-(4-methylthiazol-2-yl)-3-azabicyclo[3.1.0]hexan-6-yl)methanamine
((1R,5S,6r)-3-(3-(3-chloropyridin-4-yl)-1H-pyrazolo[3,4-b]pyrazin-6-yl)-6-(4-methylthiazol-2-yl)-3-azabicyclo[3.1.0]hexan-6-yl)methanamine
((1R,5S,6r)-3-(5-((3-chloro-2-(1H-pyrazol-1-yl)-1,4-dihydropyridin-4-yl)thio)-1,2-dihydropyrazin-2-yl)-6-(4-methylthiazol-2-yl)-3-azabicyclo[3.1.0]hexan-6-yl)methanamine
((1S,6R,7R)-7-(2-fluorophenyl)-3-(3-(8-(methylsulfonyl)quinolin-5-yl)-1H-pyrazolo[3,4-b]pyrazin-6-yl)-3-azabicyclo[4.1.0]heptan-7-yl)methanamine
((1S,6R,7R)-3-(3-(5-chloro-6-(1H-pyrrol-1-yl)-1,2-dihydropyridin-4-yl)-1H-pyrazolo[3,4-b]pyrazin-6-yl)-7-(2-fluorophenyl)-3-azabicyclo[4.1.0]heptan-7-yl)methanamine
6-((1S,6R,7R)-7-(aminomethyl)-7-(2-fluorophenyl)-3-azabicyclo[4.1.0]heptan-3-yl)-3-((5-chloroquinoxalin-6-yl)thio)pyrazin-2-amine
6-((1S,6R,7R)-7-(aminomethyl)-7-(2-fluorophenyl)-3-azabicyclo[4.1.0]heptan-3-yl)-3-(quinolin-4-ylthio)pyrazin-2-amine
5-((3-amino-5-((1S,6R,7R)-7-(aminomethyl)-7-(2-fluorophenyl)-3-azabicyclo[4.1.0]heptan-3-yl)pyrazin-2-yl)thio)quinoline-8-carboxamide
6-((1R,5S,6r)-6-(aminomethyl)-6-(4-methylthiazol-2-yl)-3-azabicyclo[3.1.0]hexan-3-yl)-5-chloro-3-(2,3-dichlorophenyl)-2-methylpyrimidin-4(3H)-one
((1S,6R,7R)-7-(2,5-difluorophenyl)-3-(6-(1-methyl-1H-benzo[d]imidazol-6-yl)pyrido[2,3-b]pyrazin-2-yl)-3-azabicyclo[4.1.0]heptan-7-yl)methanamine
((1R,5S,6R)-3-(3-(4-chloro-2-cyclopropyl-2H-indazol-5-yl)-1H-pyrazolo[3,4-b]pyrazin-6-yl)-6-(4-methylthiazol-2-yl)-3-azabicyclo[3.1.0]hexan-6-yl)methanamine
((1S,6R,7R)-3-(6-(3-fluoro-2-methylpyridin-4-yl)pyrido[2,3-b]pyrazin-2-yl)-7-(2-fluorophenyl)-3-azabicyclo[4.1.0]heptan-7-yl)methanamine
((1S,6R,7R)-3-(6-(2-(1H-pyrazol-1-yl)pyridin-4-yl)pyrido[2,3-b]pyrazin-2-yl)-7-(2-fluorophenyl)-3-azabicyclo[4.1.0]heptan-7-yl)methanamine
5-(6-((1S,6R,7R)-7-(aminomethyl)-7-(2,5-difluorophenyl)-3-azabicyclo[4.1.0]heptan-3-yl)-1H-pyrazolo[3,4-b]pyrazin-3-yl)-3,4-dihydroquinolin-2(1H)-one
or a pharmaceutically acceptable salt, tautomer, solvate, or stereoisomer thereof.

12. (canceled)

13. (canceled)

14. A method for the treatment and/or prevention of a disease or disorder mediated by the activity of SHP2, comprising administering a compound of claim 1 or the pharmaceutically acceptable salt, tautomer, solvate, or stereoisomer thereof to a patient in need thereof.

15. The method of claim 14, wherein the disease or disorder is selected from the group consisting of cancer, cardiovascular disease, immunological disorder, autoimmune disorder, fibrosis, an ocular disorder, systemic lupus erythematosus, diabetes, neutropenia and combinations thereof.

16. The method according to claim 14, wherein the disease or disorder is selected from the group consisting of Noonan Syndrome, Leopard Syndrome, juvenile myelomonocytic leukemias, neuroblastoma, melanoma, head and neck squamous-cell carcinoma, acute myeloid leukemia, breast cancer, esophageal tumor, lung cancer, colon cancer, head cancer, gastric carcinoma, lymphoma, glioblastoma, gastric cancer, pancreatic cancer and combinations thereof.

17. The method according to claim 15 wherein any one of said cancers is a primary cancer or a cancer metastasis.

18. The method according to claim 14, wherein said treatment is in combination with radiotherapy or with at least one further therapeutic agent, preferably said at least one further therapeutic agent is selected from the group consisting of:
(a) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide(TEMODAR), thiotepa, and cyclophosphamide (ENDOXAN);
(b) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX) and raltitrexed;
(c) antimitotics, which are often plant alkaloids and terpenoids, or derivatives thereof, including but not limited to taxanes such as docetaxel (TAXITERE) and paclitaxel (ABRAXANE, TAXOL); vinca alkaloids such as vincristine (ONCOVIN), vinblastine, vindesine, vinorelbine (NAVELBINE), and vinflunine;
(d) checkpoint inhibitors, such as anti-PD-1 or PD-L1 antibodies pembrolizumab (KEYTRUDA), nivolumab (OPDIVO), MEDI4736 and MPDL3280A; anti-CTLA-4 antibody ipilimumab (YERVOY); inhibitors that target LAG3 (lymphocyte activation gene 3 protein), KIR (killer cell immunoglobulin-like receptor), 4-1BB (tumour necrosis factor receptor superfamily member 9), TIM3 (T-cell immunoglobulin and mucin-domain containing-3) and/or OX40 (tumour necrosis factor receptor superfamily member 4);
(e) topoisomerase inhibitors, including but not limited to camptothecin (CTP), irinotecan (CAMPTOSAR), topotecan (HYCAMTIN), teniposide (VUMON) and etoposide (EPOSIN);
(f) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin; (7) aromatase inhibitors, including but not limited to aminoglutethimide, anastrozole (ARIMIDEX), letrozole (FEMARA), vorozole (RIVIZOR) and exemestane (AROMASIN);
(g) angiogenesis inhibitors, including but not limited to genistein, sunitinib (SUTENT) and bevacizumab (AVASTIN);
(h) anti-steroids and anti-androgens such as aminoglutethimide (CYTADREN), bicalutamide (CASODEX), cyproterone, flutamide (EULEXIN) and nilutamide (NILANDRON);
(i) tyrosine kinase inhibitors, including but not limited to imatinib (GLEEVEC), erlotinib (TARCEVA), lapatininb (TYKERB), sorafenib (NEXAVAR), and axitinib (INLYTA);
(j) mTOR inhibitors such as everolimus, temsirolimus (TORISEL), and sirolimus; (12) monoclonal antibodies such as trastuzumab (HERCEPTIN) and rituximab (RITUXAN);
(k) other agents, such as amsacrine; Bacillus Calmette-Gudrin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone(HALOTESTIN); estrogens such as estradiol, diethylstilbestrol (DES), and dienestrol; progestins such as medroxyprogesterone acetate (MPA) and megestrol; testosterone; 5-fluoro-2-4(1H,3H)-pyrimidinedione and combinations thereof.

19. A pharmaceutical composition comprising the compound or the pharmaceutically acceptable salt, tautomer, solvate, or stereoisomer thereof of claim 1, alone or in combination with a further therapeutic agent, and a pharmaceutically acceptable excipient, preferably said further therapeutic agent is selected from the group consisting of:
(a) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide(TEMODAR), thiotepa, and cyclophosphamide (ENDOXAN);
(b) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX) and raltitrexed;
(c) antimitotics, which are often plant alkaloids and terpenoids, or derivatives thereof, including but not limited to taxanes such as docetaxel (TAXITERE) and paclitaxel (ABRAXANE, TAXOL); vinca alkaloids such as vincristine (ONCOVIN), vinblastine, vindesine, vinorelbine (NAVELBINE), and vinflunine;
(d) checkpoint inhibitors, such as anti-PD-1 or PD-L1 antibodies pembrolizumab (KEYTRUDA), nivolumab (OPDIVO), MEDI4736 and MPDL3280A; anti-CTLA-4 antibody ipilimumab (YERVOY); inhibitors that target LAG3 (lymphocyte activation gene 3 protein), K1R (killer cell immunoglobulin-like receptor), 4-1BB (tumour necrosis factor receptor superfamily member 9), TIM3 (T-cell immunoglobulin and mucin-domain containing-3) and/or OX40 (tumour necrosis factor receptor superfamily member 4);
(e) topoisomerase inhibitors, including but not limited to camptothecin (CTP), irinotecan (CAMPTOSAR), topotecan (HYCAMTIN), teniposide (VUMON) and etoposide (EPOSIN);
(f) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin; (7) aromatase inhibitors, including but not limited to aminoglutethimide, anastrozole (ARIMIDEX), letrozole (FEMARA), vorozole (RIVIZOR) and exemestane (AROMASIN);
(g) angiogenesis inhibitors, including but not limited to genistein, sunitinib (SUTENT) and bevacizumab (AVASTIN);
(h) anti-steroids and anti-androgens such as aminoglutethimide (CYTADREN), bicalutamide (CASODEX), cyproterone, flutamide (EULEXIN) and nilutamide (NILANDRON);
(i) tyrosine kinase inhibitors, including but not limited to imatinib (GLEEVEC), erlotinib (TARCEVA), lapatininb (TYKERB), sorafenib (NEXAVAR), and axitinib (INLYTA);
(j) mTOR inhibitors such as everolimus, temsirolimus (TORISEL), and sirolimus; (12) monoclonal antibodies such as trastuzumab (HERCEPTIN) and rituximab (RITUXAN);
(k) other agents, such as amsacrine; Bacillus Calmette-Guérin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; testosterone; 5-fluoro-2-4(1H,3H)-pyrimidinedione and combinations thereof.

20. (canceled)
